(19) United States
(12) Patent Application Publication
(10) Pub. No.: US 2024/0239788 A1
(43) Pub. Date: Jul. 18, 2024

(54) SMALL MOLECULE INHIBITORS OF KRAS G12D MUTANT

(71) Applicant: Merck Sharp & Dohme LLC, Rahway, NJ (US)

(72) Inventors: David L. Sloman, Newton, MA (US); Gianni Chessari, Cambridge (GB); Patrick Schöpf, London (GB); Steven Howard, Cambridge (GB); Yuichi Kawai, Ibaraki (JP); Kazuaki Shibata, Ibaraki (JP); Hiroki Asakura, Ibaraki (JP); Takao Uno, Ibaraki (JP); Takeshi Sagara, Ibaraki (JP); Masayuki Nakamura, Ibaraki (JP); Yu Kobayakawa, Ibaraki (JP); David Jonathan Bennett, Winchester, MA (US); Indu Bharathan, Somerville, MA (US); Thomas H. Graham, Somerville, MA (US); Yongxin Han, Needham, MA (US); Zahid Hussain, Dayton, NJ (US); Xiaoshen Ma, Boston, MA (US); Mihir Mandal, Westfield, NJ (US); Ryan D. Otte, Natick, MA (US); Anandan Palani, Needham, MA (US); Uma Swaminathan, Auburndale, MA (US); Mycah Uehling, New Brunswick, NJ (US); Yingchun Ye, Belmont, MA (US); Ryan Chau, Somerville, MA (US); Alec H. Christian, Sharon, MA (US); Symon Gathiaka, Waltham, MA (US); Timothy J. Henderson, Natick, MA (US); Elisabeth T. Hennessy, Weston, MA (US); Andrew J. Hoover, Framingham, MA (US); Shuhei Kawamura, Cambridge, MA (US); Igri Kolaj, Brighton, MA (US); Thomas W. Lyons, Brookline, MA (US); Matthew J. Mitcheltree, Jamaica Plain, MA (US); Aaron Sather, Melrose, MA (US)

(21) Appl. No.: 18/555,640

(22) PCT Filed: Apr. 15, 2022
(86) PCT No.: PCT/US2022/025132

§ 371 Date: Oct. 16, 2023

Related U.S. Application Data
(60) Provisional application No. 63/176,135, filed on Apr. 16, 2021.

(51) Int. Cl. C07D 451/06; A61K 31/517; A61K 31/519; A61K 31/5377; A61K 45/06; C07D 471/08; C07D 519/00

(52) U.S. Cl. CPC C07D 451/06; A61K 31/517; A61K 31/519; A61K 31/5377; A61K 45/06; C07D 471/08; C07D 519/00

(57) ABSTRACT

Compounds or their pharmaceutically acceptable salts can inhibit the G12D mutant of Kirsten rat sarcoma (KRAS) protein and are expected to have utility as therapeutic agents, for example, for treating cancer. The disclosure also provides pharmaceutical compositions which comprise compounds disclosed herein or pharmaceutically acceptable salts thereof. The disclosure also relates to methods for use of the compounds or their pharmaceutically acceptable salts in the therapy and prophylaxis of cancer and for preparing pharmaceuticals for this purpose.

The present application claims the benefit of U.S. Provisional Application No. 63/176,135, filed Apr. 16, 2021, the entirety of which is incorporated by reference herein.

### BACKGROUND OF THE INVENTION

### Field of the Invention

The present disclosure relates to certain compounds and pharmaceutically acceptable salts thereof that inhibit the G12D mutant of Kirsten rat sarcoma (KRAS) protein and are expected to have utility as therapeutic agents, for example, for treatment of cancer. The present application also relates to pharmaceutical compositions containing such compounds as well as methods of using the compounds for treating cancer.

Mutations in any one of the three main isoforms of RAS (HRAS, NRAS, or KRAS) genes are among the most common events in human tumorigenesis. KRAS mutations are detected in 25-30% of tumors. By comparison, the rates of oncogenic mutation occurring in the NRAS and HRAS family members are much lower (8% and 3% respectively).

Ras proteins are membrane-associated guanine nucleotide-binding proteins which function as molecular switches. Ras proteins function as components of signaling pathways transmitting signals from cell-surface receptors to regulate cellular proliferation, survival and differentiation. Ras proteins cycle between an inactive GDP-bound state and an active GTP-bound state.

Exchange of a glycine for an aspartate at residue 12 of RAS (the G12D mutation) results from a gain-of-function mutation commonly found in RAS gene. The K-Ras(G12D) mutation represents the highest frequency of KRAS mutations. Wan Y. et al., Front. Oncol. 2020; 10:1326. The K-Ras(G12D) mutation appears frequently in certain cancer types including pancreatic ductal adenocarcinoma, colorectal cancer and lung adenocarcinoma. Pancreatic ductal adenocarcinoma is particularly significant. This cancer type represents a malignancy having a 5-year survival rate of less than 8% and is also the fourth leading cause of cancer-related deaths in the western world. See Schneeweis C. et al., Small GTPases 2018; 9(6) 457-464.

Accordingly, while progress has been made in this field, there remains a need in the art for improved compounds and methods for treatment of cancer, for example, by inhibition of a mutant KRAS, HRAS or NRAS protein (e.g., KRAS G12D). Embodiments of the present disclosure fulfill this need and provide further related advantages.

### SUMMARY OF THE DISCLOSURE

The present disclosure provides fused bicyclic pyrimidines which modulate mutant KRAS, HRAS, and/or NRAS proteins and may be valuable pharmaceutically active compounds for the treatment of cancer. In some embodiments the disclosed compounds selectively inhibit the KRAS (G12D) protein. The compounds of the disclosure, including compounds of Formula (I)

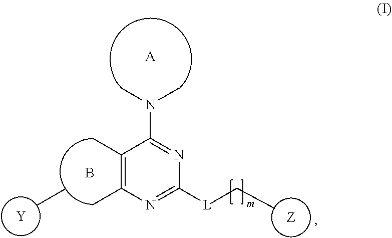

and their pharmaceutically acceptable salts, can modulate the activity of KRAS, HRAS and/or NRAS activity and thereby affect the signaling pathway which regulates cell growth, differentiation, and proliferation associated with oncological disorders. In certain embodiments, the compounds of the disclosure can inhibit the KRAS (G12D) protein. The disclosure furthermore provides processes for preparing compounds of the disclosure, methods for using such compounds to treat oncological disorders, and pharmaceutical compositions which comprise compounds of the disclosure.

### DETAILED DESCRIPTION OF THE INVENTION

### Compounds of the Disclosure

In one embodiment, the present disclosure provides a compound having structural Formula (I) as shown above wherein:
Ring A is a saturated or partially unsaturated 8- to 10-membered N-containing bridged ring which contains at least one further heteroatom selected from the group consisting of N, S and O:
wherein Ring A is unsubstituted or substituted by 1 to 3R^{A} substituents selected from the group consisting of C₁-C₃ alkyl, C₂-C₄ alkenyl, C₁-C₃ alkoxy, C₁-C₃ alkoxy(C₁-C₃)alkyl, halo, C₁-C₃ fluoroalkyl, hydroxy, C₁-C₃ hydroxyalkyl, CF₃—C(H)(OH)—, C(H)(F₂)—C(H)(OH)—, cyano, and C₁-C₃ cyanoalkyl;
Ring B is a 5- or 6-membered partially unsaturated or aromatic ring having 0, 1 or 2 heteroatoms selected from the group consisting of N. S, and O and wherein Ring B is fused with the illustrated pyrimidine ring;
wherein Ring B is unsubstituted or substituted by 1 to 2R^{B} substituents selected from the group consisting of halo, hydroxy, oxo, cyano, C₁-C₃ alkyl, C I-C₃ fluoroalkyl, and C₁-C₃ alkoxy;
Ring Y is a 6-membered mono-, a 9- to 10-membered bicyclic-, or a 13- to 14-membered tricyclic ring system, wherein said ring system is partially unsaturated or aromatic, and wherein Ring Y contains 0 to 3 heteroatoms selected from the group consisting of N, S, and O;
wherein Ring Y is unsubstituted or substituted by 1 to 4R^{Y} substituents selected from the group consisting of halo, hydroxy, oxo, C₁-C₃ alkyl, C₂-C₃ alkynyl, C₁-C₃ fluoroalkyl, C₁-C₃ alkoxy, C₁-C₃ fluoroalkoxy, C₁-C₃ alkylthio, C₁-C₃ fluoroalkylthio, amino, C₁-C₃ alkylamino, C₁-C₃ dialkylamino, C₃-C₁₂ cycloalkyl, tri(C₁-C₃ alkyl)silyl, and cyano;
Ring Z is
(i) a 3- to 10-membered mono- or bicyclic cycloalkyl;
(ii) a 3- to 10-membered mono- or bicyclic-heterocycloalkyl, wherein said heterocycloalkyl is saturated and contains 1 to 2 heteroatoms selected from the group consisting of N, S, and O; or
(iii) a 3- to 8-membered spiroheterocycloalkyl, wherein said spiroheterocycloalkyl is saturated and contains 1 to 2 heteroatoms selected from the group consisting of N, S, and 0;
wherein Ring Z is unsubstituted or substituted by 1 to 4R^{Z} substituents selected from the group consisting of halo, hydroxy, C₁-C₆ alkyl, C₁-C₃ alkoxy, C₁-C₃ hydroxyalkyl, C₁-C₆ fluoroalkyl, carboxy, carbamoyl, methoxy(C₁-C₃)alkyl, amino(C₁-C₃)alkyl. C₁-C₃ alkylamino(C₁-C₃)alkyl. C₁-C₃ dialkylamino, and C₁-C₃ dialkylamino(C₁-C₃)alkyl;
Ring Z is optionally substituted by 1-M-R^{ZC}, wherein
M is —CH₂— or absent; and
R^{ZC} is a 5- to 6-membered mono- or a 9- to 10-membered bicyclic saturated heterocycloalkyl which contains 1 to 3 heteroatoms selected from the group consisting of N, S, and O, wherein R^{ZC} is unsubstituted or substituted by a substituent selected from the group consisting of C₁-C₃ alkyl, C₁-C₃ alkylcarbonylalkyl, C₁-C₃ hydroxyalkyl, fluoro, cyano, amino. C₁-C₃ alkylamino, C₁-C₃ dialkylamino, C₁-C₃ alkoxyalkyl, and C₁-C₃ cyanoalkyl;
L is O or absent;
subscript m is 0, 1, or 2; and
with the proviso that when Ring B is

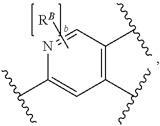

then
Ring A is substituted by at least 1 to 2R^{A} substituents selected from the group consisting of C₁-C₃ alkyl and C₂-C₄ alkenyl and
is optionally substituted by 1 to 2 substituents selected from the group consisting of C₁-C₃ alkoxy. C₁-C₃ alkoxy(C₁-C₃)alkyl, halo, hydroxy, cyano, and C₁-C₃ cyanoalkyl,
or a pharmaceutically acceptable salt thereof.

In another embodiment, the present disclosure provides a compound of Formula (I) w % herein Ring A is:

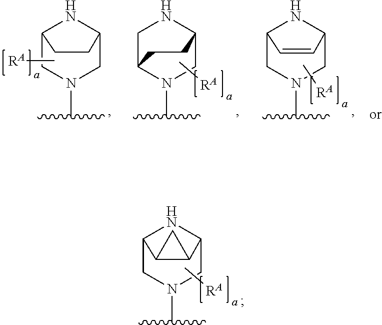

wherein subscript a is 0, 1, or 2.

In certain embodiments, Ring A is substituted by 1 to 2R^{A} substituents selected from methyl, ethyl, hydroxy, fluoro, ethenyl, methoxy, cyano, cyanomethyl, and methoxymethyl.

In another embodiment, the present disclosure provides a compound of Formula (I) wherein Ring A is:

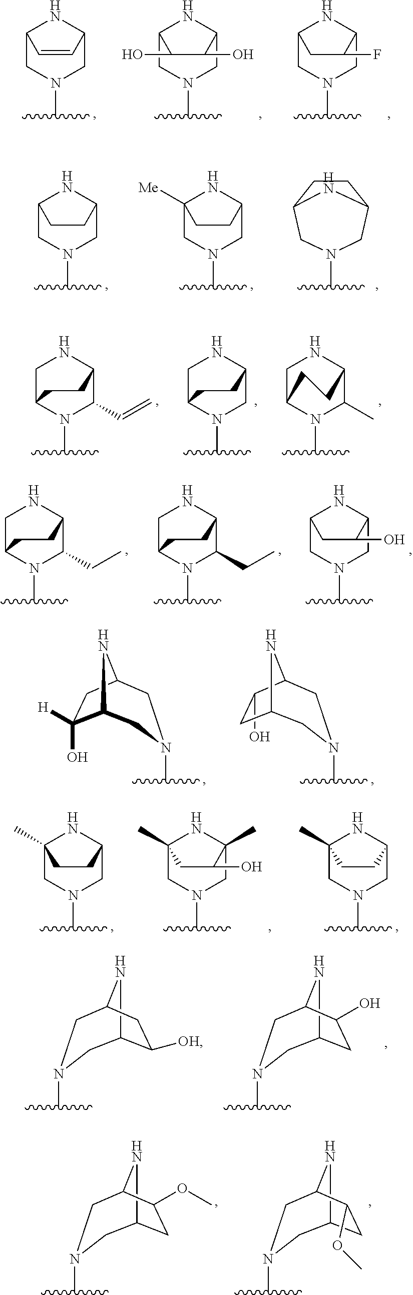

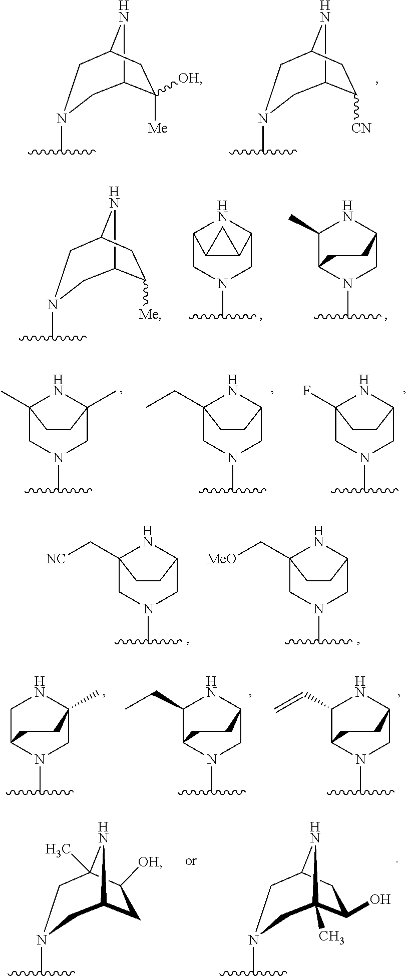

In another embodiment, the present disclosure provides a compound of Formula (I), wherein Ring A is:

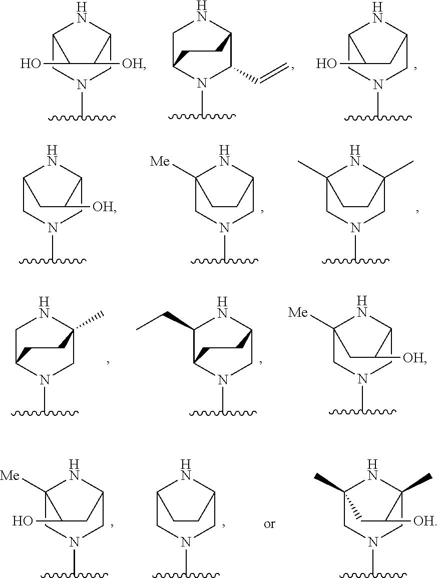

In some embodiments, the present disclosure provides a compound of Formula (I), wherein Ring B is;

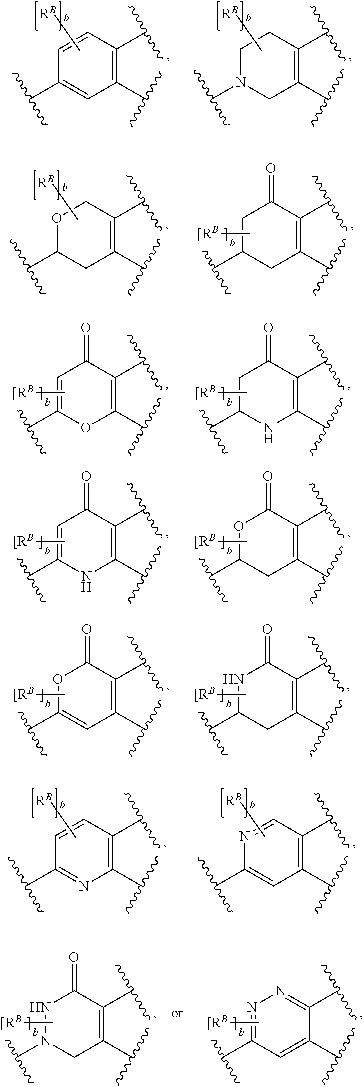

wherein subscript b is 0 or 1.

In certain embodiments, the present disclosure provides a compound of Formula (I), where the moiety

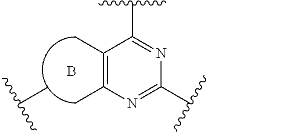

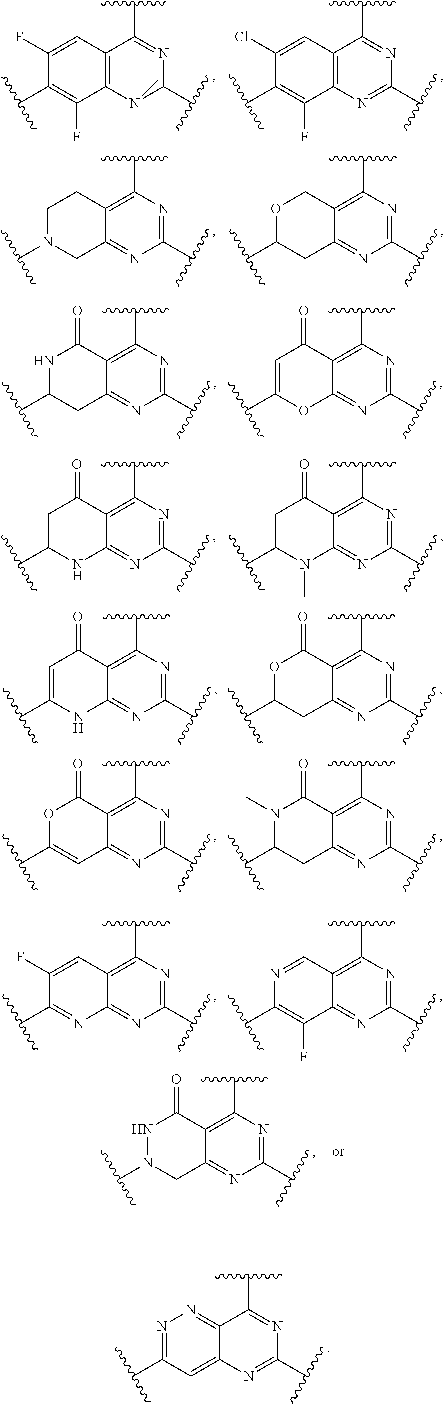

In some embodiments, the present disclosure provides a compound of Formula (I), wherein Ring Y is:

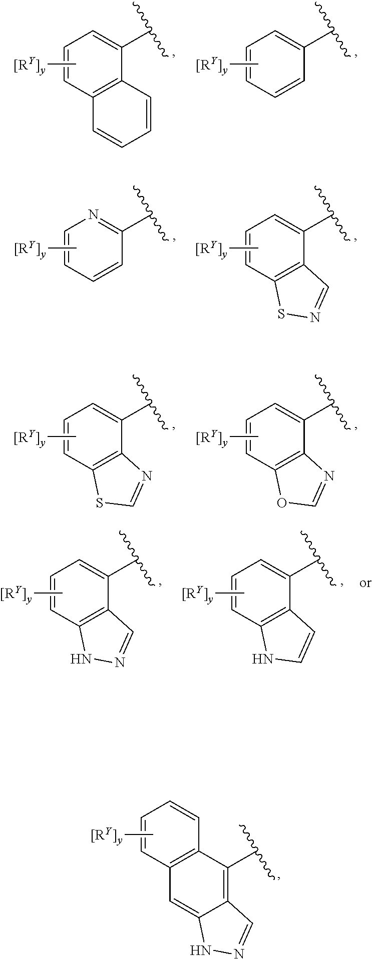

wherein subscript y is 0, 1, 2, 3, or 4.

In certain embodiments, the present disclosure provides a compound of Formula (I), wherein Ring Y is:

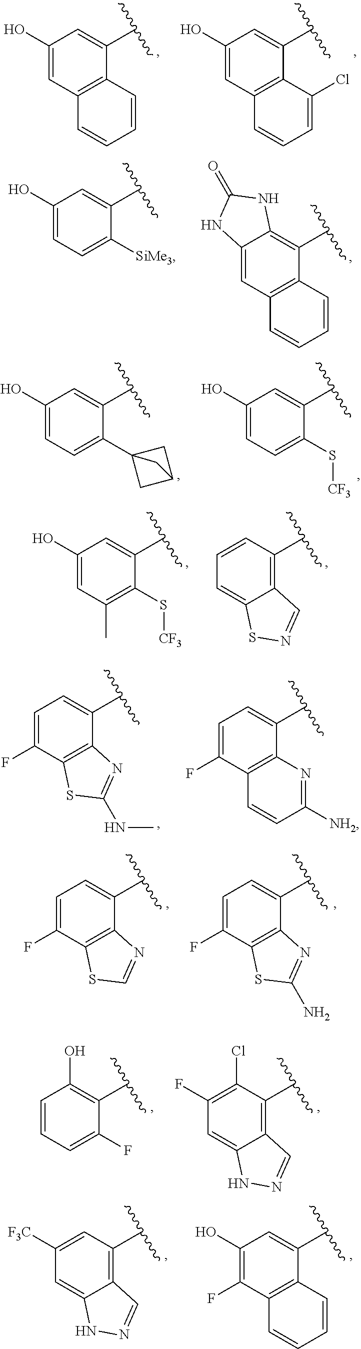

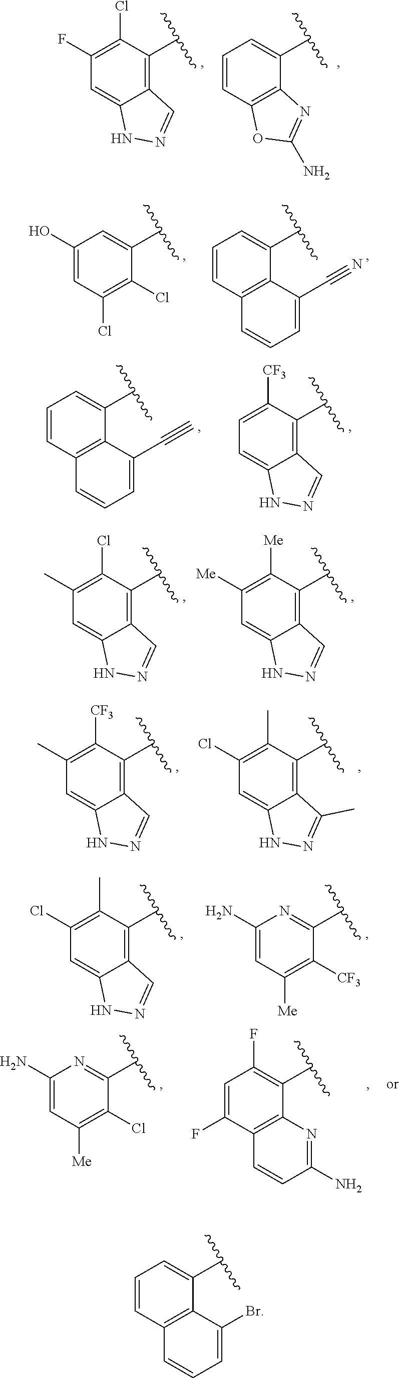

In specific embodiments, the present disclosure provides a compound of Formula (I), wherein Ring Y is:

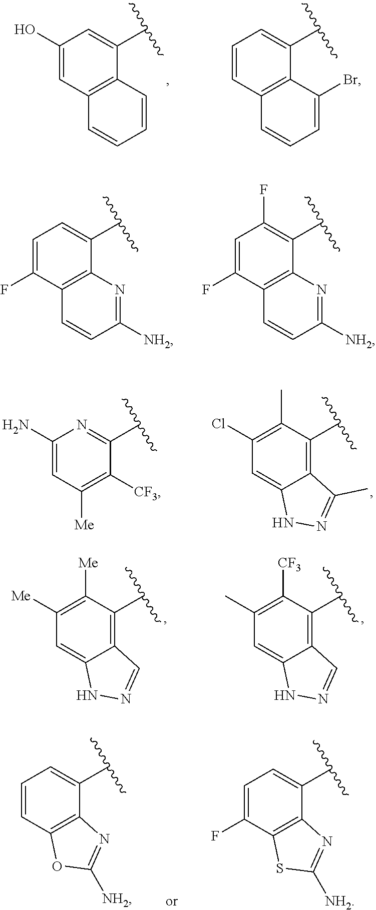

In some embodiments, the present disclosure provides a compound of Formula (I), wherein the subscript m is 1.

In certain embodiments, the present disclosure provides a compound of Formula (I), wherein the moiety

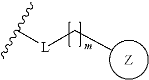

is:

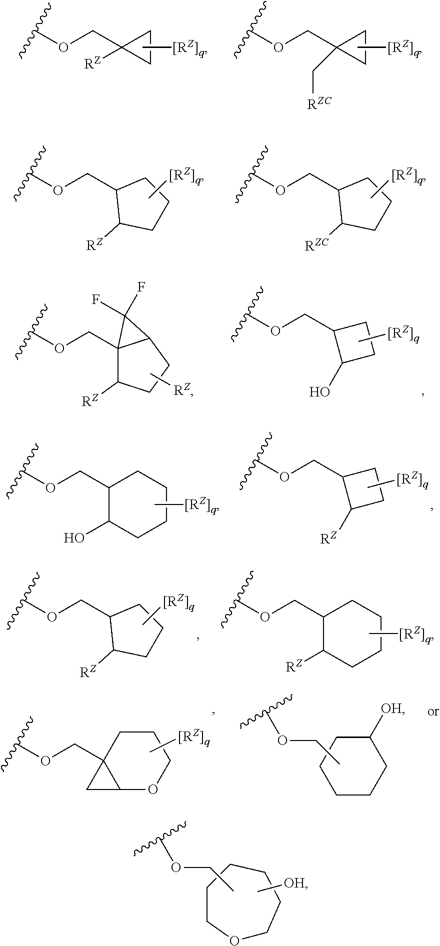

wherein subscript q is 0, 1, 2, or 3.

In embodiments, wherein ring Z is substituted, i.e., with R^{Z} or R^{ZC}, the ring Z substituent may be substituted at any ring atom, including the ring joining ring Z with L.

In specific embodiments, the moiety

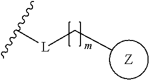

is:

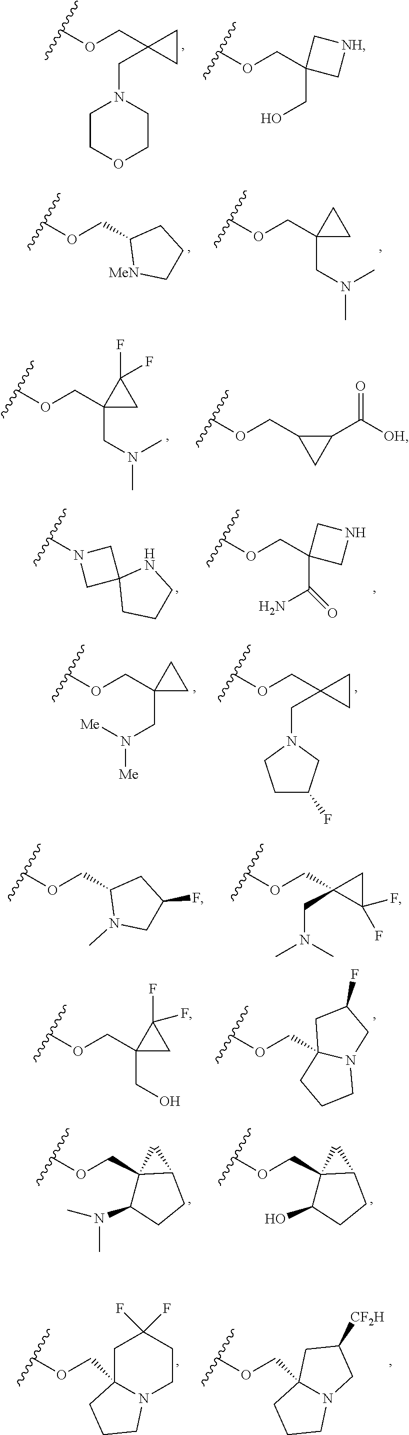

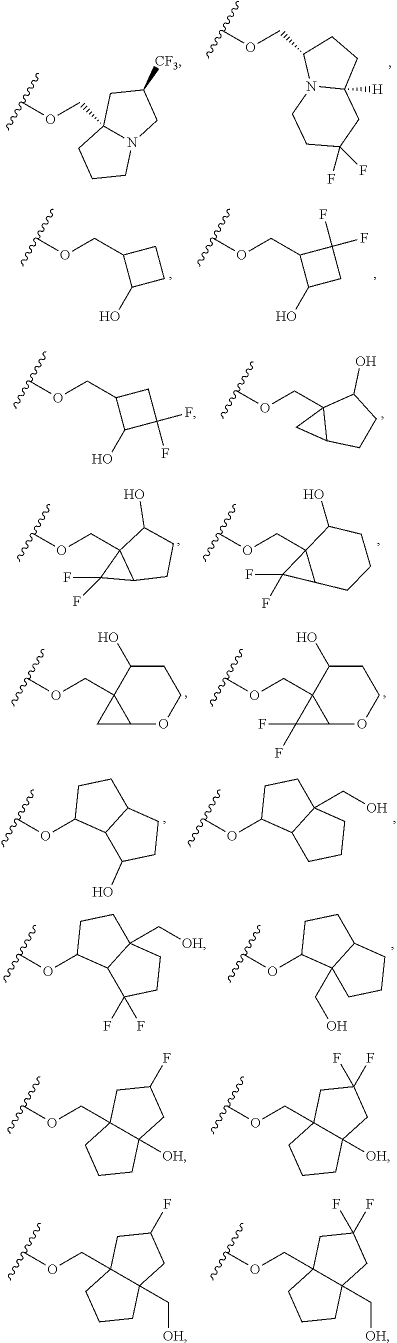

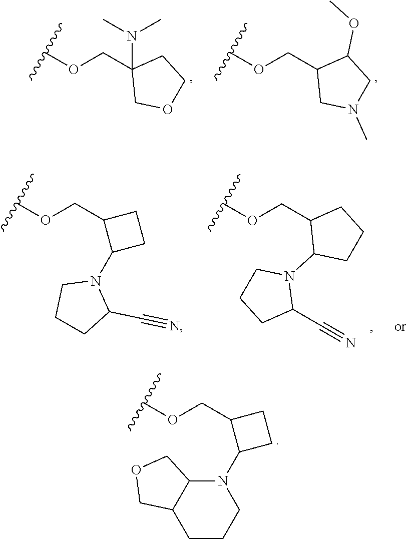 [SMILES: *OCC1(N(C)C)CCOC1 ; *OCC1CN(C)CC1OC ; *OCC1CCC1N1CCCC1C#N ; *OCC1CCCC1N1CCCC1C#N ; *OCC1CCC1N1CCCC2COCC21]

In specific embodiments, the moiety

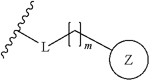

is:

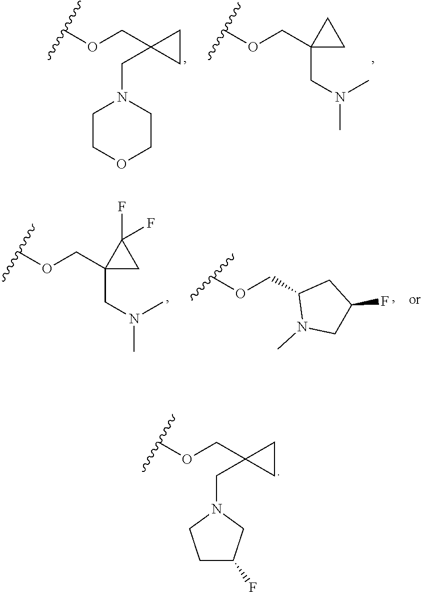

In some embodiments, the present disclosure provides a compound of Formula (I), wherein:
Ring A is:

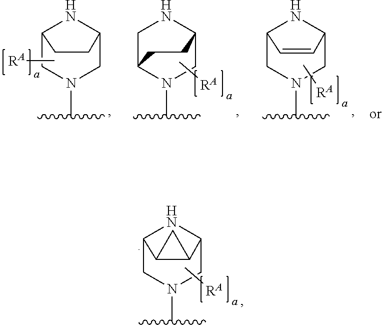

wherein subscript a is 0, 1, or 2:
Ring B is:

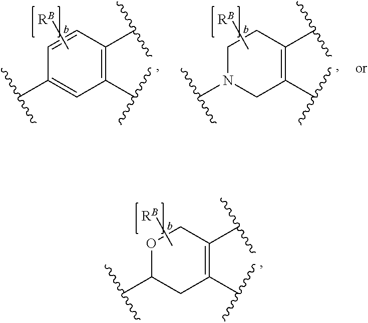

wherein subscript b is 0, 1, or 2;
Ring Y is:

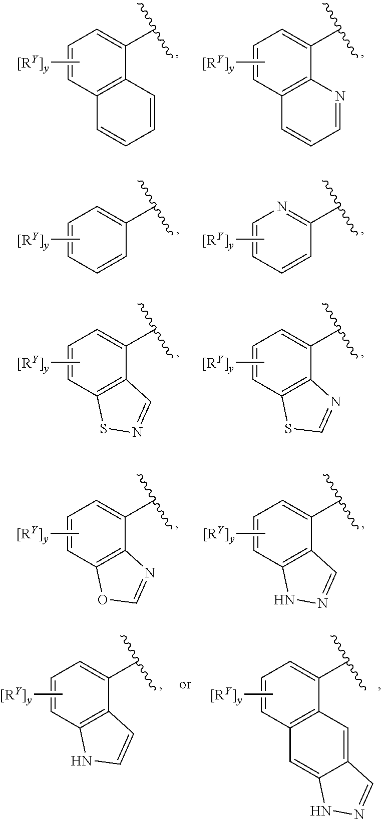

wherein subscript y is 0, 1, 2, 3, or 4;
the subscript m is 1;
Ring Z is cyclopropyl, azetidine, or pyrrolidine;
wherein Ring Z is unsubstituted or substituted by 1 to 3R^{Z} substituents selected from the group consisting of fluoro, C₁-C₃ alkyl, C₁-C₃ hydroxyalkyl, C₁-C₃ fluoroalkyl, C₁-C₃ dialkylamino(C₁-C₃)alkyl, carboxy, and carbamoyl; and
Ring Z is optionally substituted by 1-M-R^{ZC}, wherein
M is —CH₂— or absent; and
R^{ZC} is a 5- or 6-membered saturated heterocycloalkyl which contains 1 to 2 heteroatoms selected from the group consisting of N, S, and O, wherein R^{ZC} is unsubstituted or substituted by a substituent selected from the group consisting of C₁-C₃ alkyl, fluoro, and cyano.

In specific embodiments, the present disclosure provides a compound of Formula (I), wherein
Ring A is:

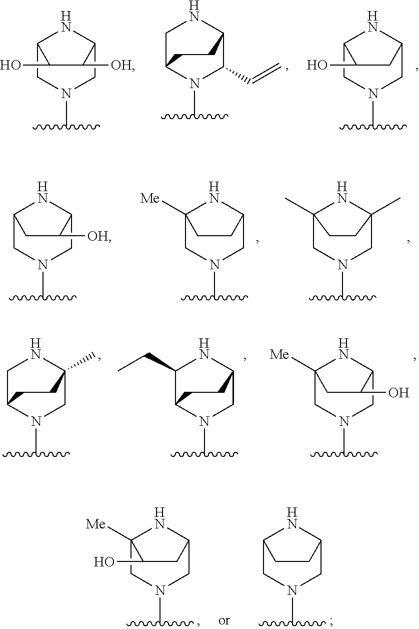

the moiety

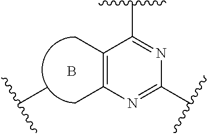

is:

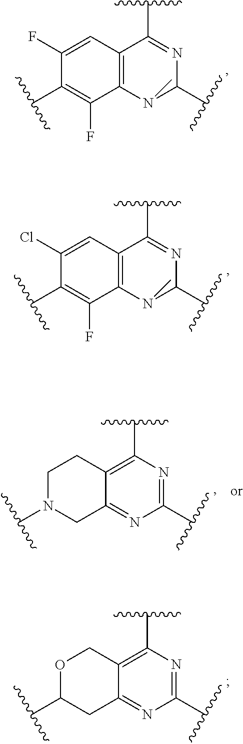

### Ring Y is;

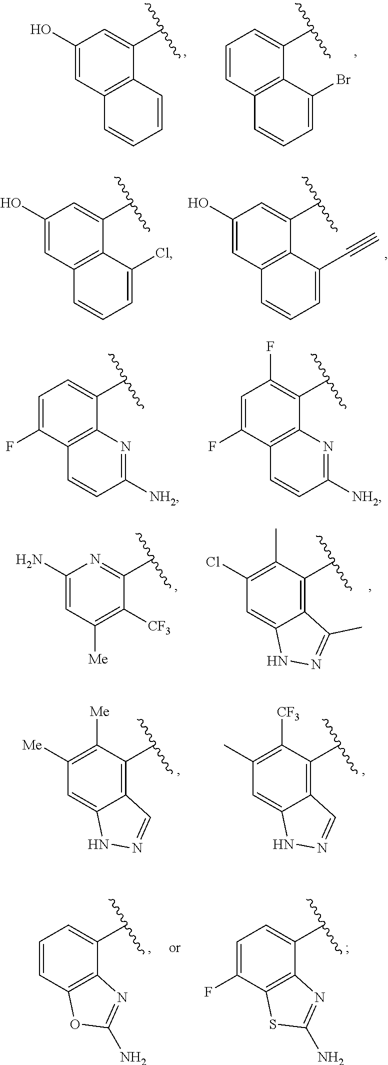

and the moiety

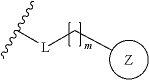

is

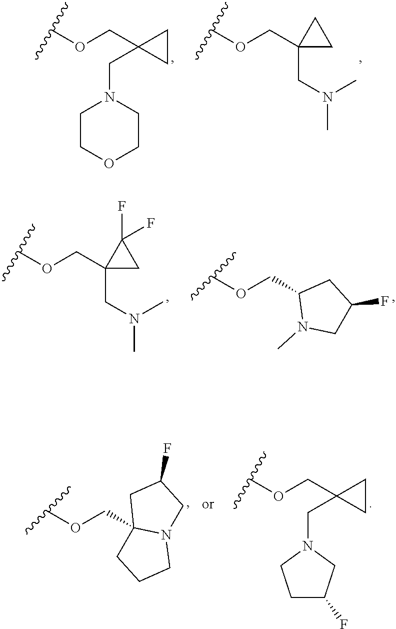

In certain embodiments, the present disclosure provides a compound as PGP-46 described in any one of Examples 1-256 as set forth below, or a pharmaceutically acceptable salt thereof.

In certain embodiments, the present disclosure provides a compound or a pharmaceutically acceptable salt thereof selected from the group consisting of;

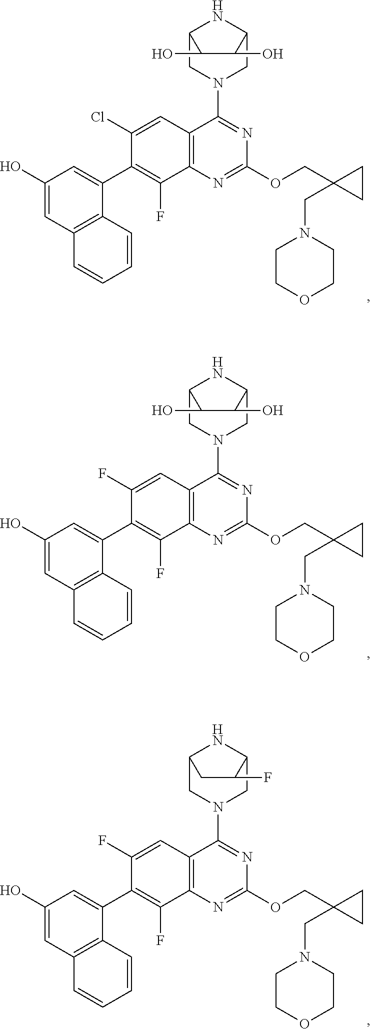 [SMILES: Oc1cc(-c2c(Cl)cc3c(N4CC5NC(C4)C(O)C5O)nc(OCC4(CN5CCOCC5)CC4)nc3c2F)c2ccccc2c1 ; Oc1cc(-c2c(F)cc3c(N4CC5NC(C4)C(O)C5O)nc(OCC4(CN5CCOCC5)CC4)nc3c2F)c2ccccc2c1 ; Oc1cc(-c2c(F)cc3c(N4CC5CC(F)C(C4)N5)nc(OCC4(CN5CCOCC5)CC4)nc3c2F)c2ccccc2c1]

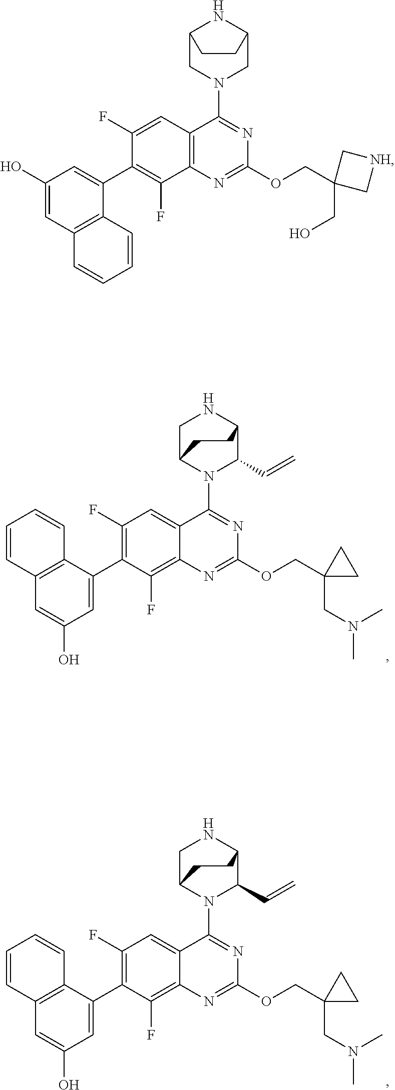 [SMILES: OCC1(COc2nc(N3CC4CCC(C3)N4)c3cc(F)c(-c4cc(O)cc5ccccc45)c(F)c3n2)CNC1 ; C=C[C@H]1[C@@H]2CC[C@@H](CN2)N1c1nc(OCC2(CN(C)C)CC2)nc2c(F)c(-c3cc(O)cc4ccccc34)c(F)cc12 ; C=C[C@@H]1[C@@H]2CC[C@@H](CN2)N1c1nc(OCC2(CN(C)C)CC2)nc2c(F)c(-c3cc(O)cc4ccccc34)c(F)cc12]

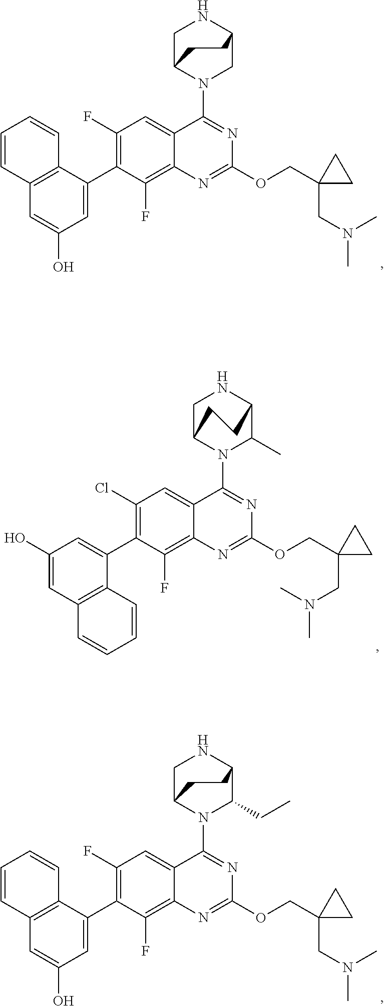 [SMILES: CN(C)CC1(COc2nc(N3C[C@@H]4CC[C@H]3CN4)c3cc(F)c(-c4cc(O)cc5ccccc45)c(F)c3n2)CC1 ; CC1[C@@H]2CC[C@@H](CN2)N1c1nc(OCC2(CN(C)C)CC2)nc2c(F)c(-c3cc(O)cc4ccccc34)c(Cl)cc12 ; CC[C@H]1[C@@H]2CC[C@@H](CN2)N1c1nc(OCC2(CN(C)C)CC2)nc2c(F)c(-c3cc(O)cc4ccccc34)c(F)cc12]

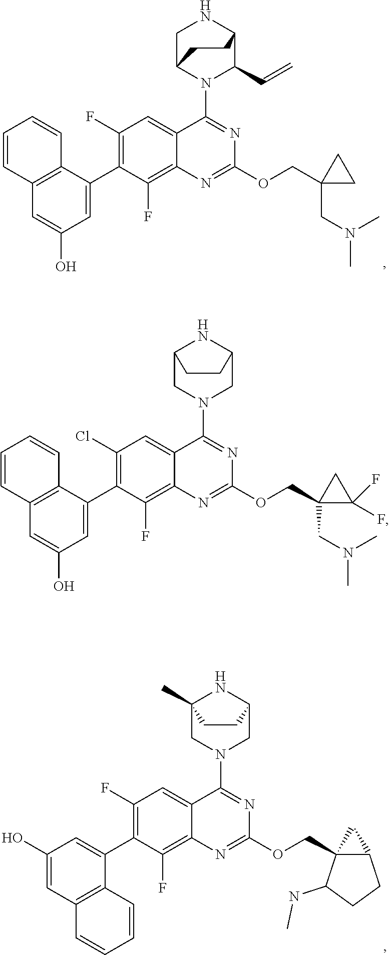

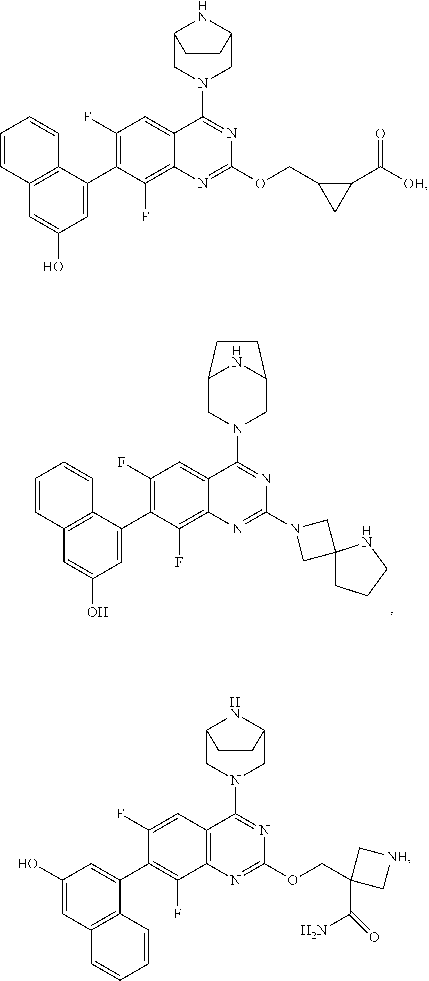 [SMILES: O=C(O)C1CC1COc1nc(N2CC3CCC(C2)N3)c2cc(F)c(-c3cc(O)cc4ccccc34)c(F)c2n1 ; Oc1cc(-c2c(F)cc3c(N4CC5CCC(C4)N5)nc(N4CC5(CCCN5)C4)nc3c2F)c2ccccc2c1 ; NC(=O)C1(COc2nc(N3CC4CCC(C3)N4)c3cc(F)c(-c4cc(O)cc5ccccc45)c(F)c3n2)CNC1]

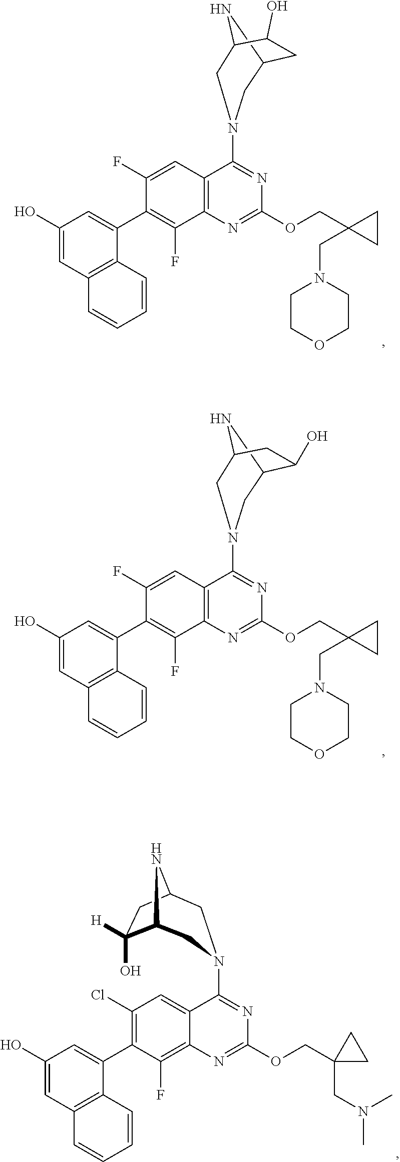

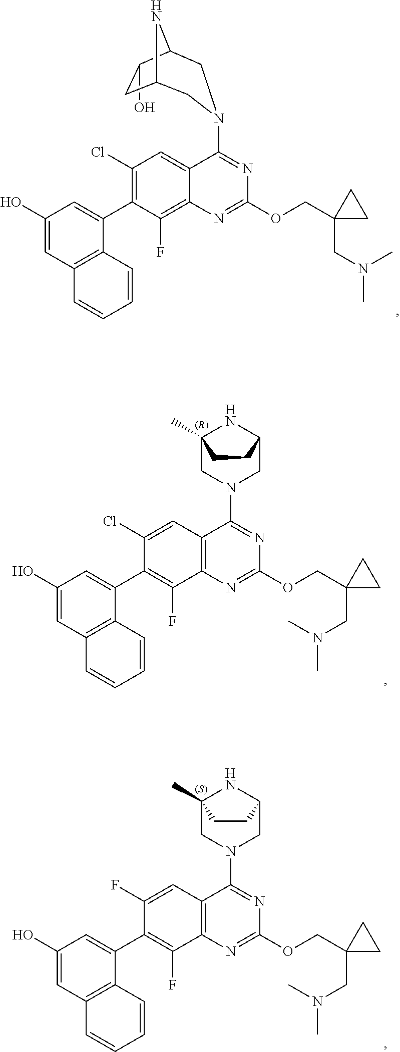

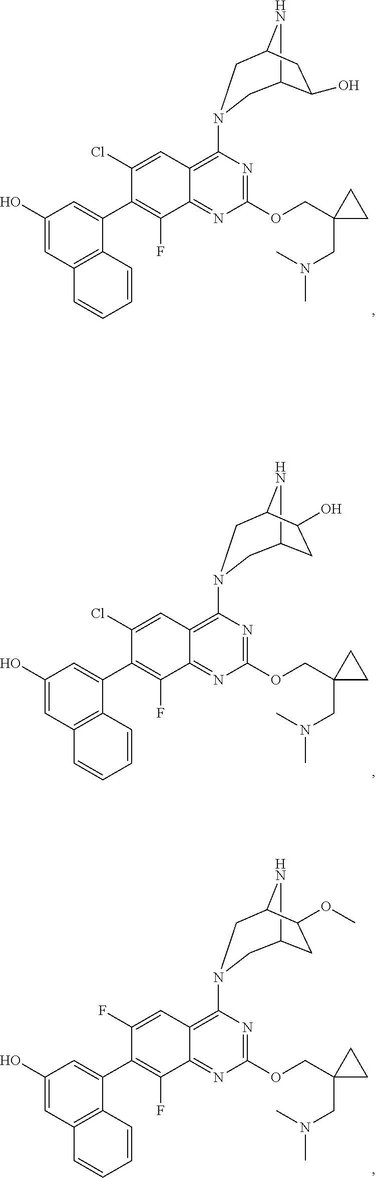 [SMILES: CN(C)CC1(COc2nc(N3CC4CC(O)C(C3)N4)c3cc(Cl)c(-c4cc(O)cc5ccccc45)c(F)c3n2)CC1 ; CN(C)CC1(COc2nc(N3CC4CC(O)C(C3)N4)c3cc(Cl)c(-c4cc(O)cc5ccccc45)c(F)c3n2)CC1 ; COC1CC2CN(c3nc(OCC4(CN(C)C)CC4)nc4c(F)c(-c5cc(O)cc6ccccc56)c(F)cc34)CC1N2]

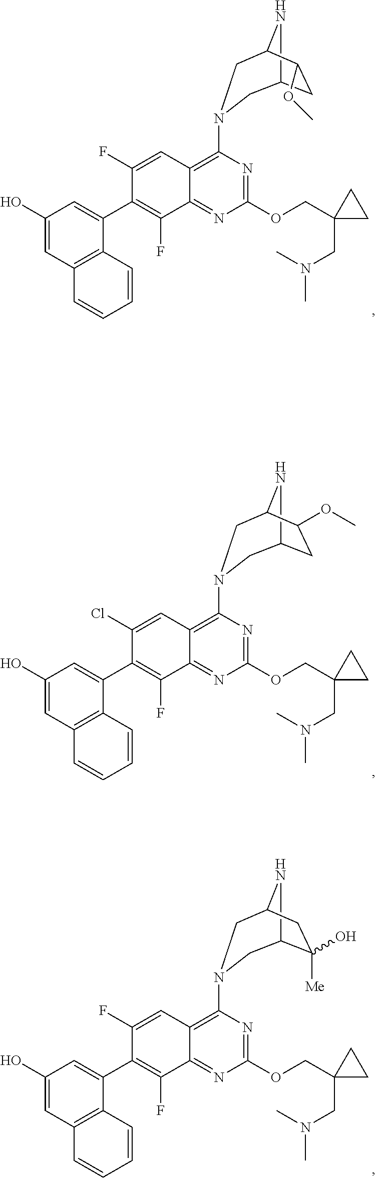 [SMILES: COC1CC2CN(c3nc(OCC4(CN(C)C)CC4)nc4c(F)c(-c5cc(O)cc6ccccc56)c(F)cc34)CC1N2 ; COC1CC2CN(c3nc(OCC4(CN(C)C)CC4)nc4c(F)c(-c5cc(O)cc6ccccc56)c(Cl)cc34)CC1N2 ; CN(C)CC1(COc2nc(N3CC4CC(C)(O)C(C3)N4)c3cc(F)c(-c4cc(O)cc5ccccc45)c(F)c3n2)CC1]

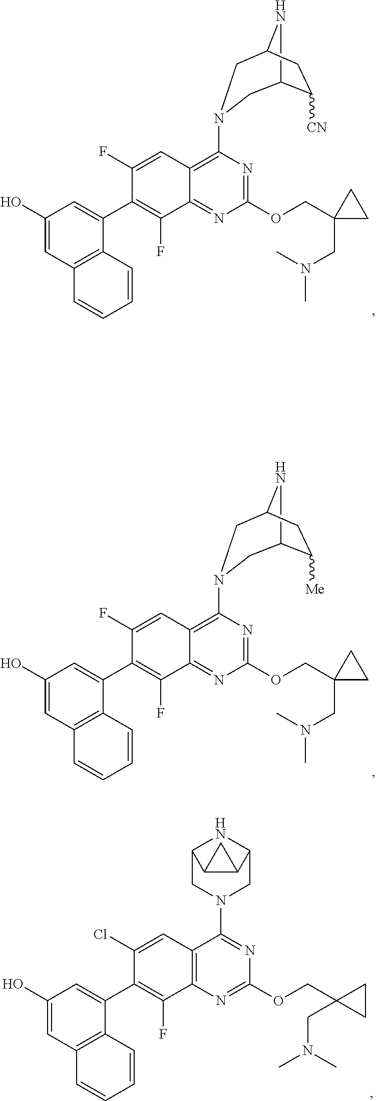 [SMILES: CN(C)CC1(COc2nc(N3CC4CC(C#N)C(C3)N4)c3cc(F)c(-c4cc(O)cc5ccccc45)c(F)c3n2)CC1 ; CC1CC2CN(c3nc(OCC4(CN(C)C)CC4)nc4c(F)c(-c5cc(O)cc6ccccc56)c(F)cc34)CC1N2 ; CN(C)CC1(COc2nc(N3CC4NC(C3)C3CC43)c3cc(Cl)c(-c4cc(O)cc5ccccc45)c(F)c3n2)CC1]

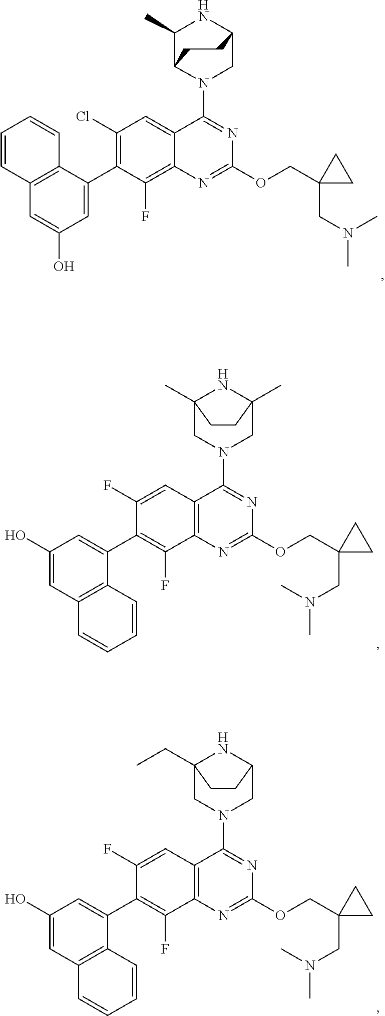 [SMILES: C[C@H]1N[C@H]2CC[C@@H]1N(c1nc(OCC3(CN(C)C)CC3)nc3c(F)c(-c4cc(O)cc5ccccc45)c(Cl)cc13)C2 ; CN(C)CC1(COc2nc(N3CC4(C)CCC(C)(C3)N4)c3cc(F)c(-c4cc(O)cc5ccccc45)c(F)c3n2)CC1 ; CCC12CCC(CN(c3nc(OCC4(CN(C)C)CC4)nc4c(F)c(-c5cc(O)cc6ccccc56)c(F)cc34)C1)N2]

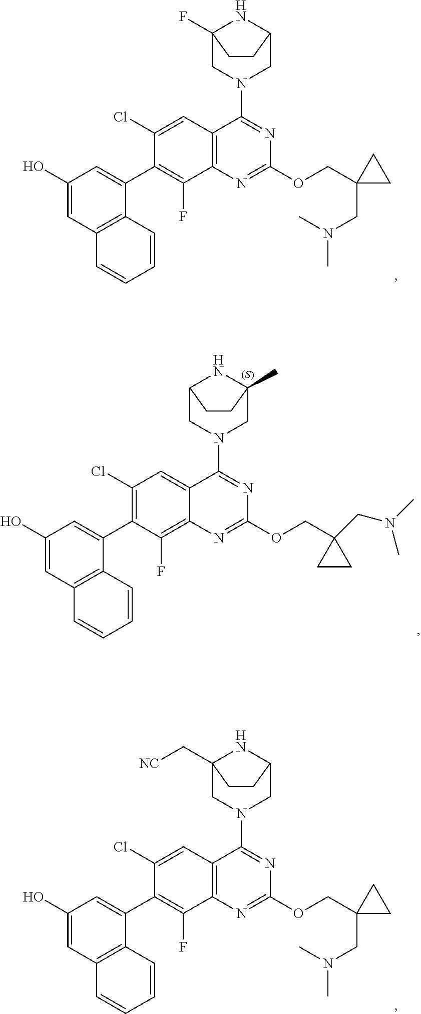

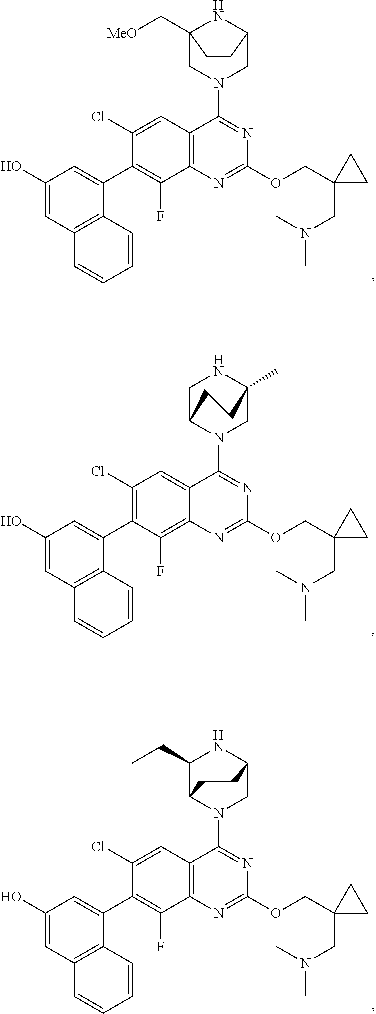 [SMILES: COCC12CCC(CN(c3nc(OCC4(CN(C)C)CC4)nc4c(F)c(-c5cc(O)cc6ccccc56)c(Cl)cc34)C1)N2 ; CN(C)CC1(COc2nc(N3C[C@]4(C)CC[C@H]3CN4)c3cc(Cl)c(-c4cc(O)cc5ccccc45)c(F)c3n2)CC1 ; CC[C@H]1N[C@H]2CC[C@@H]1N(c1nc(OCC3(CN(C)C)CC3)nc3c(F)c(-c4cc(O)cc5ccccc45)c(Cl)cc13)C2]

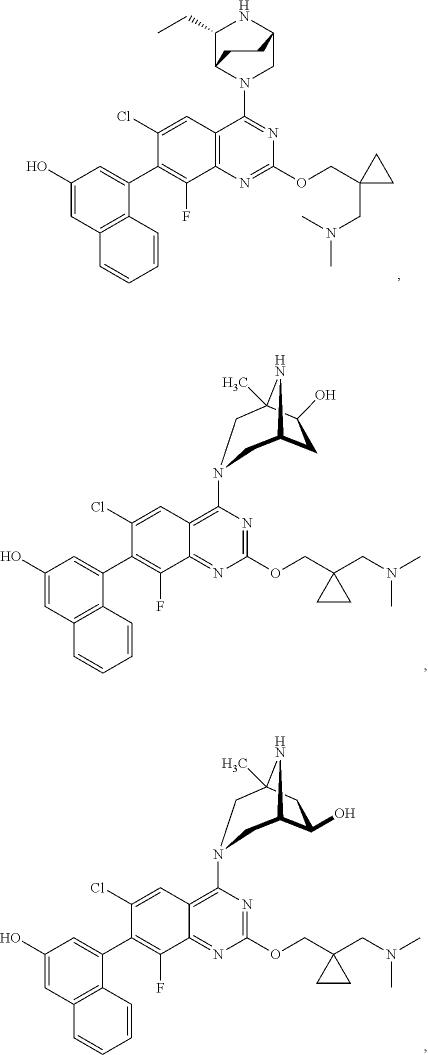

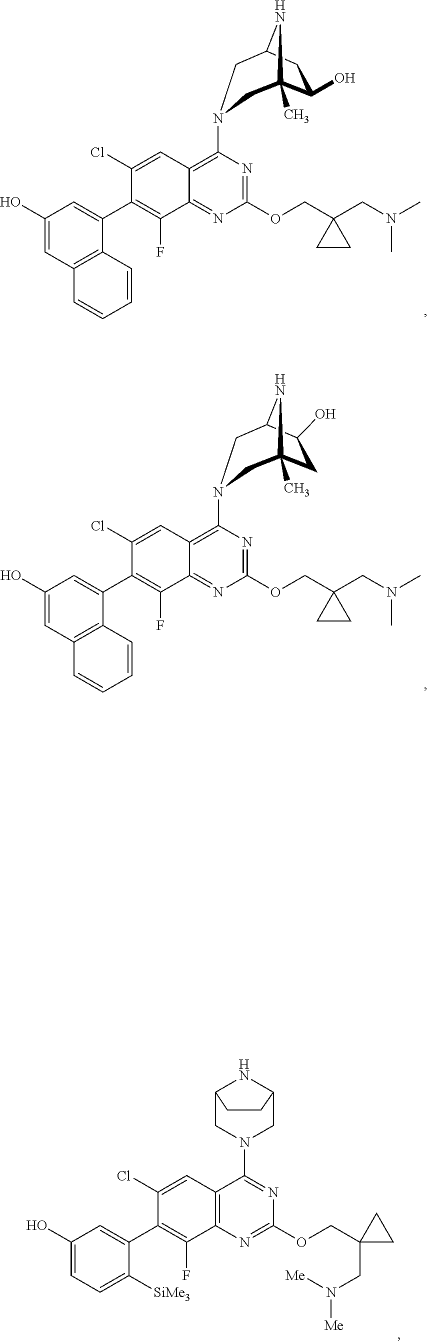

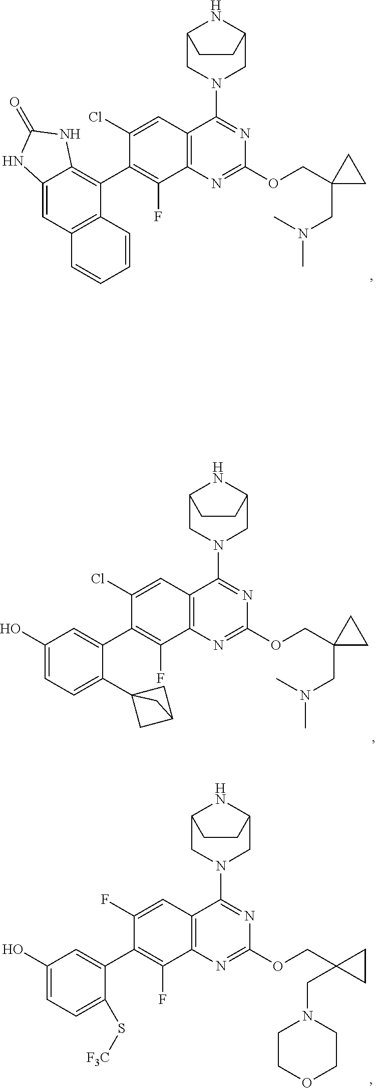 [SMILES: CN(C)CC1(COc2nc(N3CC4CCC(C3)N4)c3cc(Cl)c(-c4c5ccccc5cc5[nH]c(=O)[nH]c45)c(F)c3n2)CC1 ; CN(C)CC1(COc2nc(N3CC4CCC(C3)N4)c3cc(Cl)c(-c4cc(O)ccc4C45CC(C4)C5)c(F)c3n2)CC1 ; Oc1ccc(SC(F)(F)F)c(-c2c(F)cc3c(N4CC5CCC(C4)N5)nc(OCC4(CN5CCOCC5)CC4)nc3c2F)c1]

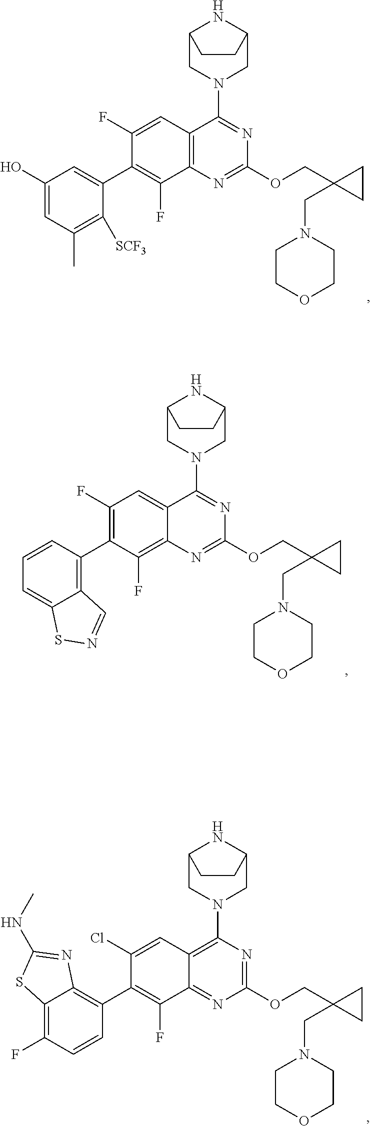

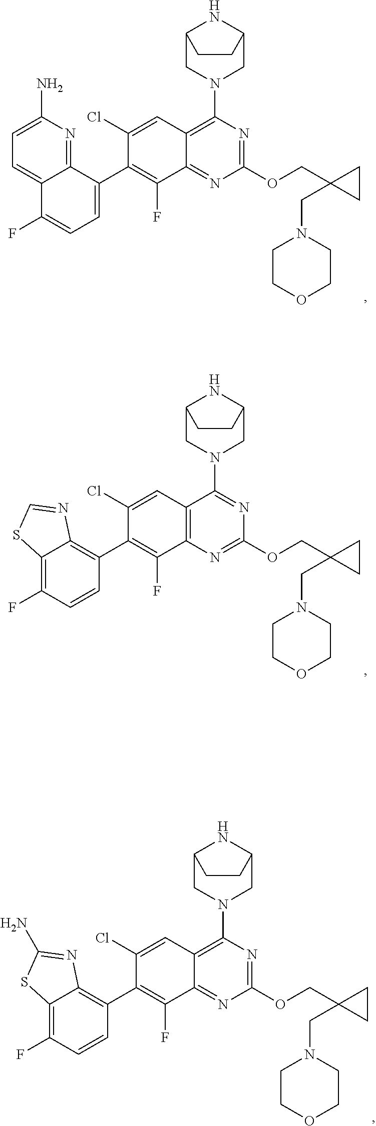 [SMILES: Nc1ccc2c(F)ccc(-c3c(Cl)cc4c(N5CC6CCC(C5)N6)nc(OCC5(CN6CCOCC6)CC5)nc4c3F)c2n1 ; Fc1c(-c2ccc(F)c3scnc23)c(Cl)cc2c(N3CC4CCC(C3)N4)nc(OCC3(CN4CCOCC4)CC3)nc12 ; Nc1nc2c(-c3c(Cl)cc4c(N5CC6CCC(C5)N6)nc(OCC5(CN6CCOCC6)CC5)nc4c3F)ccc(F)c2s1]

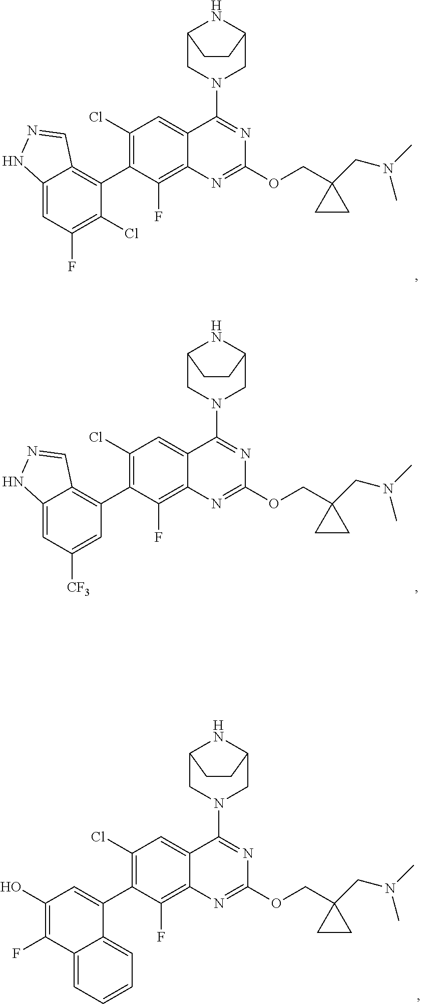 [SMILES: CN(C)CC1(COc2nc(N3CC4CCC(C3)N4)c3cc(Cl)c(-c4c(Cl)c(F)cc5[nH]ncc45)c(F)c3n2)CC1 ; CN(C)CC1(COc2nc(N3CC4CCC(C3)N4)c3cc(Cl)c(-c4cc(C(F)(F)F)cc5[nH]ncc45)c(F)c3n2)CC1 ; CN(C)CC1(COc2nc(N3CC4CCC(C3)N4)c3cc(Cl)c(-c4cc(O)c(F)c5ccccc45)c(F)c3n2)CC1]

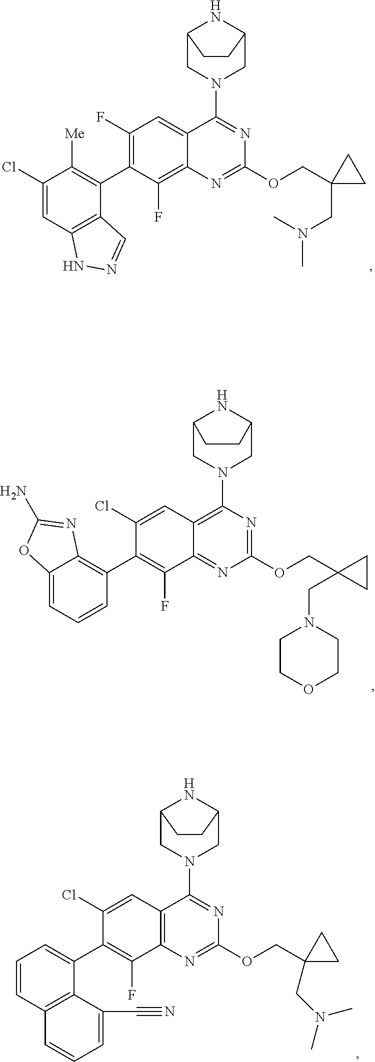 [SMILES: Cc1c(Cl)cc2[nH]ncc2c1-c1c(F)cc2c(N3CC4CCC(C3)N4)nc(OCC3(CN(C)C)CC3)nc2c1F ; Nc1nc2c(-c3c(Cl)cc4c(N5CC6CCC(C5)N6)nc(OCC5(CN6CCOCC6)CC5)nc4c3F)cccc2o1 ; CN(C)CC1(COc2nc(N3CC4CCC(C3)N4)c3cc(Cl)c(-c4cccc5cccc(C#N)c45)c(F)c3n2)CC1]

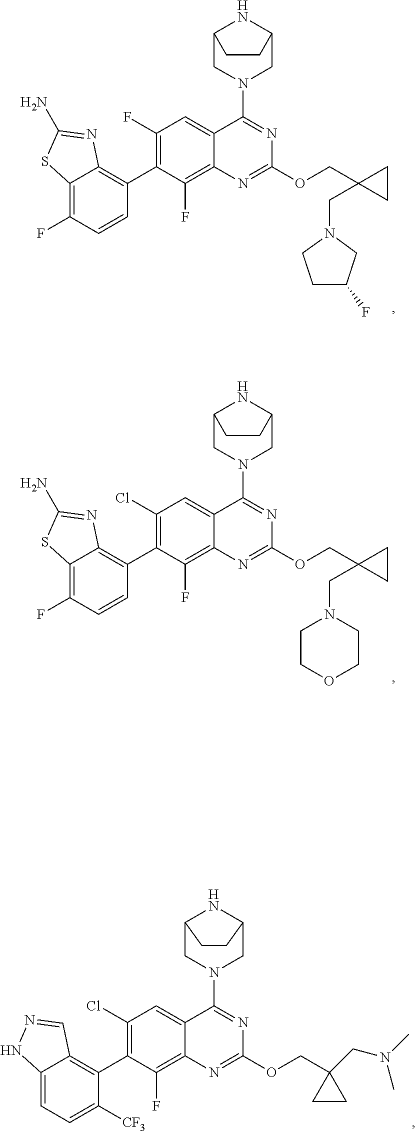 [SMILES: Nc1nc2c(-c3c(F)cc4c(N5CC6CCC(C5)N6)nc(OCC5(CN6CC[C@@H](F)C6)CC5)nc4c3F)ccc(F)c2s1 ; Nc1nc2c(-c3c(Cl)cc4c(N5CC6CCC(C5)N6)nc(OCC5(CN6CCOCC6)CC5)nc4c3F)ccc(F)c2s1 ; CN(C)CC1(COc2nc(N3CC4CCC(C3)N4)c3cc(Cl)c(-c4c(C(F)(F)F)ccc5[nH]ncc45)c(F)c3n2)CC1]

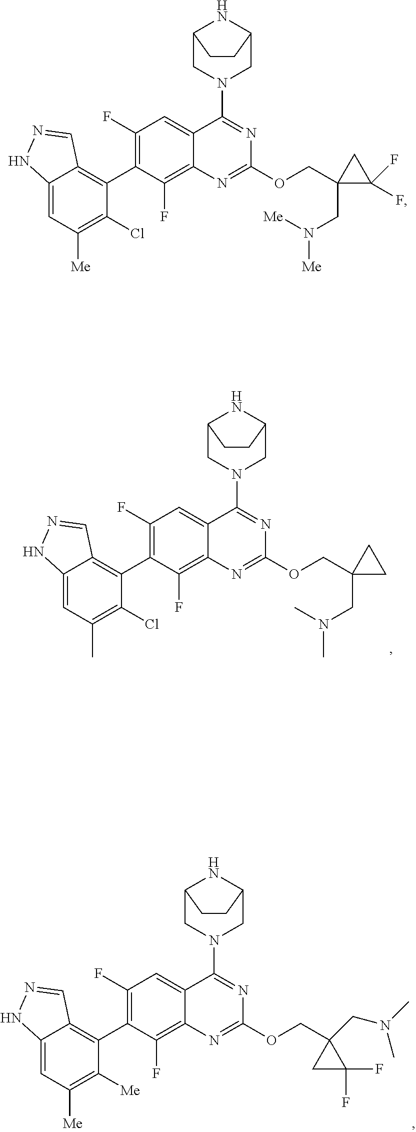 [SMILES: Cc1cc2[nH]ncc2c(-c2c(F)cc3c(N4CC5CCC(C4)N5)nc(OCC4(CN(C)C)CC4(F)F)nc3c2F)c1Cl ; Cc1cc2[nH]ncc2c(-c2c(F)cc3c(N4CC5CCC(C4)N5)nc(OCC4(CN(C)C)CC4)nc3c2F)c1Cl ; Cc1cc2[nH]ncc2c(-c2c(F)cc3c(N4CC5CCC(C4)N5)nc(OCC4(CN(C)C)CC4(F)F)nc3c2F)c1C]

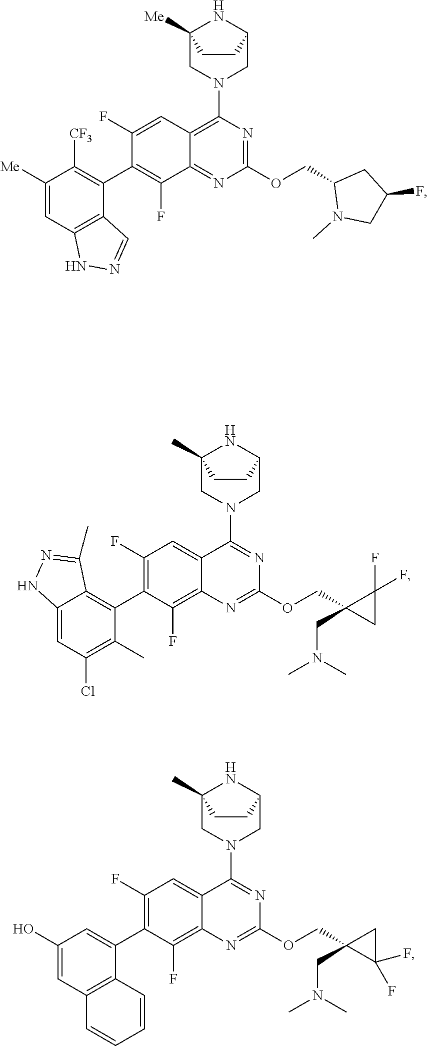 [SMILES: Cc1cc2[nH]ncc2c(-c2c(F)cc3c(N4C[C@H]5CC[C@@](C)(C4)N5)nc(OC[C@@H]4C[C@@H](F)CN4C)nc3c2F)c1C(F)(F)F ; Cc1c(Cl)cc2[nH]nc(C)c2c1-c1c(F)cc2c(N3C[C@H]4CC[C@@](C)(C3)N4)nc(OC[C@@]3(CN(C)C)CC3(F)F)nc2c1F ; CN(C)C[C@@]1(COc2nc(N3C[C@H]4CC[C@@](C)(C3)N4)c3cc(F)c(-c4cc(O)cc5ccccc45)c(F)c3n2)CC1(F)F]

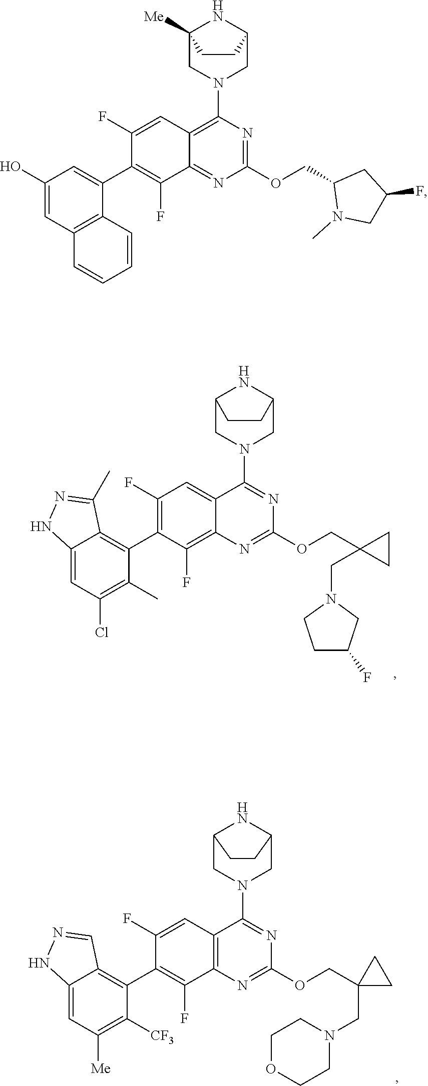 [SMILES: CN1C[C@H](F)C[C@H]1COc1nc(N2C[C@H]3CC[C@@](C)(C2)N3)c2cc(F)c(-c3cc(O)cc4ccccc34)c(F)c2n1 ; Cc1c(Cl)cc2[nH]nc(C)c2c1-c1c(F)cc2c(N3CC4CCC(C3)N4)nc(OCC3(CN4CC[C@@H](F)C4)CC3)nc2c1F ; Cc1cc2[nH]ncc2c(-c2c(F)cc3c(N4CC5CCC(C4)N5)nc(OCC4(CN5CCOCC5)CC4)nc3c2F)c1C(F)(F)F]

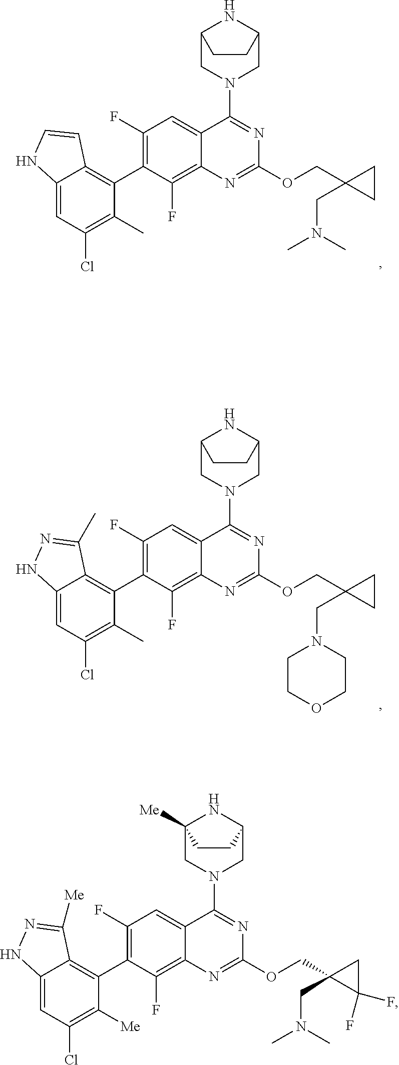 [SMILES: Cc1c(Cl)cc2[nH]ccc2c1-c1c(F)cc2c(N3CC4CCC(C3)N4)nc(OCC3(CN(C)C)CC3)nc2c1F ; Cc1c(Cl)cc2[nH]nc(C)c2c1-c1c(F)cc2c(N3CC4CCC(C3)N4)nc(OCC3(CN4CCOCC4)CC3)nc2c1F ; Cc1c(Cl)cc2[nH]nc(C)c2c1-c1c(F)cc2c(N3C[C@H]4CC[C@@](C)(C3)N4)nc(OC[C@]3(CN(C)C)CC3(F)F)nc2c1F]

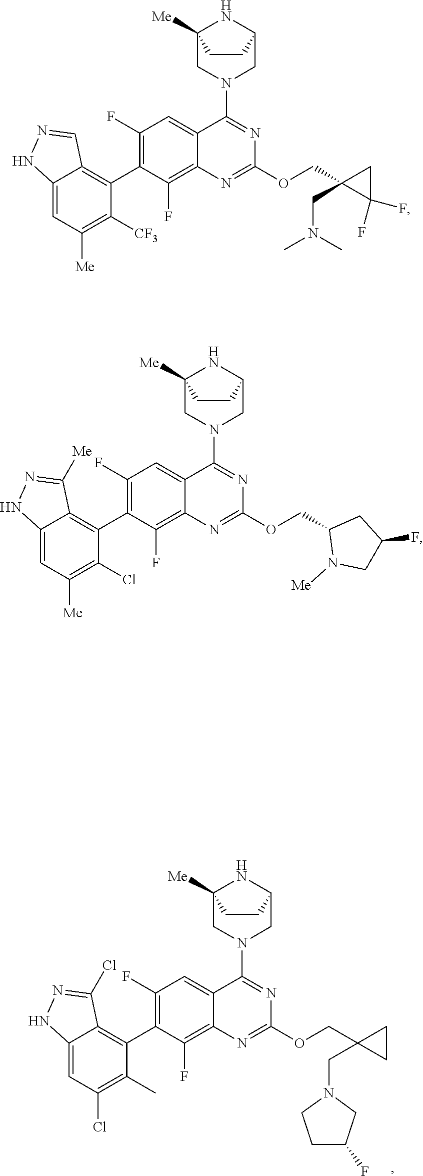 [SMILES: Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C(F)(F)F ; Cc1cc2[nH]nc(C)c2c(-c2c(F)cc3c(N4C[C@H]5CC[C@@](C)(C4)N5)nc(OC[C@@H]4C[C@@H](F)CN4C)nc3c2F)c1Cl ; Cc1c(Cl)cc2[nH]nc(Cl)c2c1-c1c(F)cc2c(N3C[C@H]4CC[C@@](C)(C3)N4)nc(OCC3(CN4CC[C@@H](F)C4)CC3)nc2c1F]

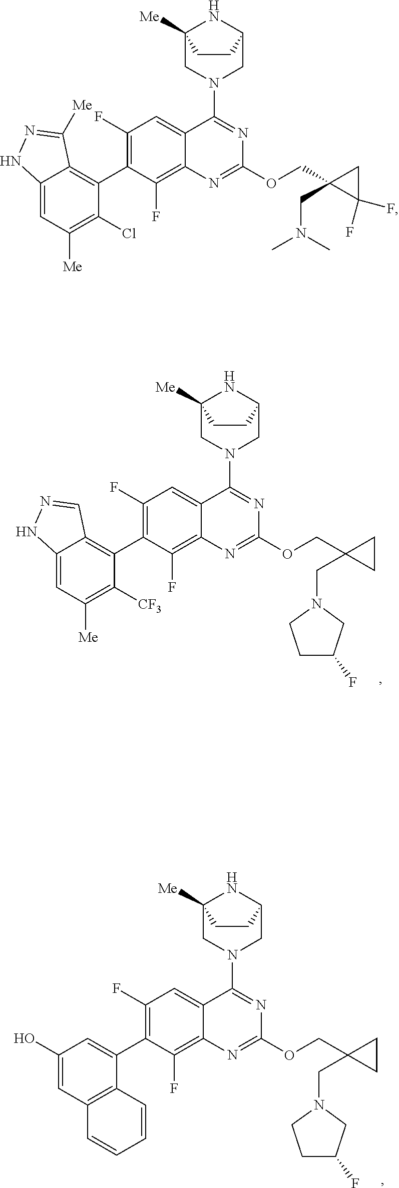 [SMILES: Cc1cc2[nH]nc(C)c2c(-c2c(F)cc3c(N4C[C@H]5CC[C@@](C)(C4)N5)nc(OC[C@]4(CN(C)C)CC4(F)F)nc3c2F)c1Cl ; Cc1cc2[nH]ncc2c(-c2c(F)cc3c(N4C[C@H]5CC[C@@](C)(C4)N5)nc(OCC4(CN5CC[C@@H](F)C5)CC4)nc3c2F)c1C(F)(F)F ; C[C@]12CC[C@H](CN(c3nc(OCC4(CN5CC[C@@H](F)C5)CC4)nc4c(F)c(-c5cc(O)cc6ccccc56)c(F)cc34)C1)N2]

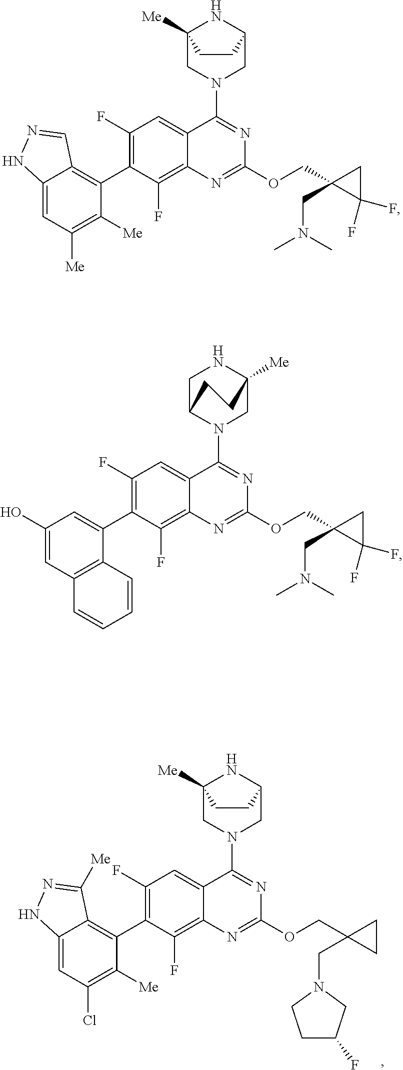 [SMILES: Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C ; CN(C)C[C@@]1(COc2nc(N3C[C@]4(C)CC[C@H]3CN4)c3cc(F)c(-c4cc(O)cc5ccccc45)c(F)c3n2)CC1(F)F ; Cc1c(Cl)cc2[nH]nc(C)c2c1-c1c(F)cc2c(N3C[C@H]4CC[C@@](C)(C3)N4)nc(OCC3(CN4CC[C@@H](F)C4)CC3)nc2c1F]

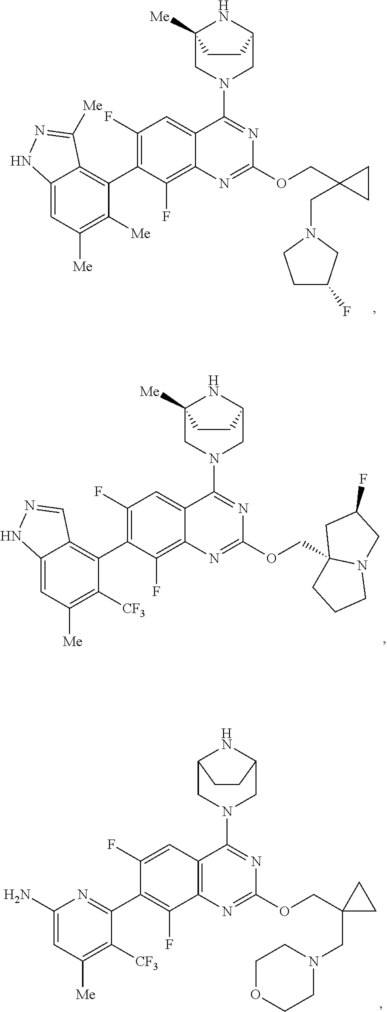 [SMILES: Cc1cc2[nH]nc(C)c2c(-c2c(F)cc3c(N4C[C@H]5CC[C@@](C)(C4)N5)nc(OCC4(CN5CC[C@@H](F)C5)CC4)nc3c2F)c1C ; Cc1cc2[nH]ncc2c(-c2c(F)cc3c(N4C[C@H]5CC[C@@](C)(C4)N5)nc(OC[C@@]45CCCN4C[C@H](F)C5)nc3c2F)c1C(F)(F)F ; Cc1cc(N)nc(-c2c(F)cc3c(N4CC5CCC(C4)N5)nc(OCC4(CN5CCOCC5)CC4)nc3c2F)c1C(F)(F)F]

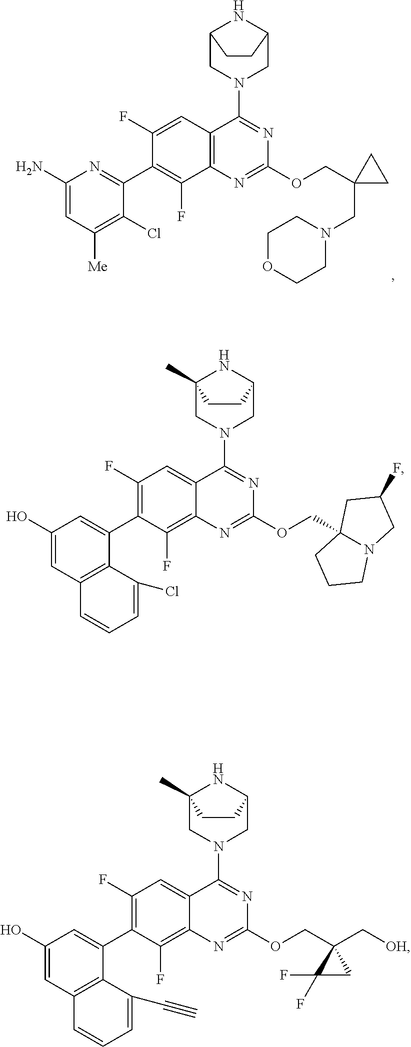 [SMILES: Cc1cc(N)nc(-c2c(F)cc3c(N4CC5CCC(C4)N5)nc(OCC4(CN5CCOCC5)CC4)nc3c2F)c1Cl ; C[C@]12CC[C@H](CN(c3nc(OC[C@@]45CCCN4C[C@H](F)C5)nc4c(F)c(-c5cc(O)cc6cccc(Cl)c56)c(F)cc34)C1)N2 ; C#Cc1cccc2cc(O)cc(-c3c(F)cc4c(N5C[C@H]6CC[C@@](C)(C5)N6)nc(OC[C@]5(CO)CC5(F)F)nc4c3F)c12]

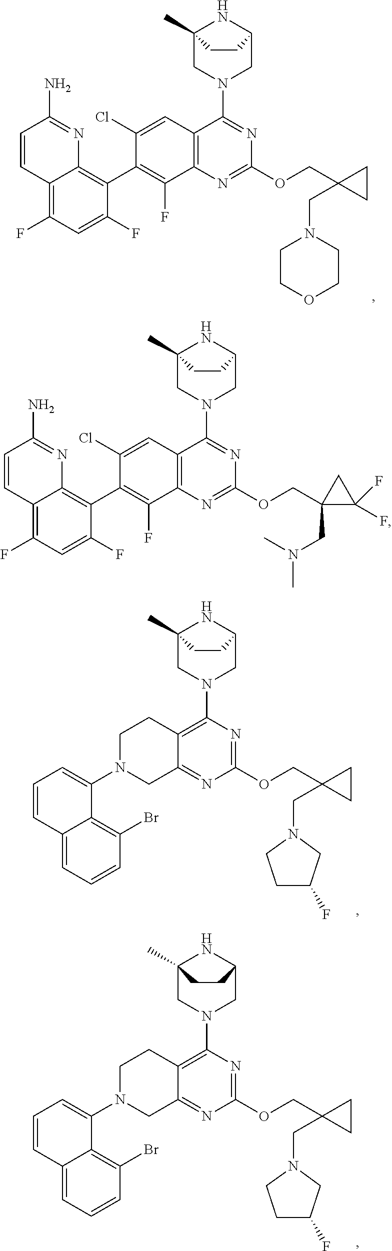 [SMILES: C[C@@]12CC[C@H](CN(c3nc(OCC4(CN5CCOCC5)CC4)nc4c(F)c(-c5c(F)cc(F)c6ccc(N)nc56)c(Cl)cc34)C1)N2 ; CN(C)C[C@@]1(COc2nc(N3C[C@H]4CC[C@](C)(C3)N4)c3cc(Cl)c(-c4c(F)cc(F)c5ccc(N)nc45)c(F)c3n2)CC1(F)F ; C[C@@]12CC[C@H](CN(c3nc(OCC4(CN5CC[C@@H](F)C5)CC4)nc4c3CCN(c3cccc5cccc(Br)c35)C4)C1)N2 ; C[C@]12CC[C@@H](CN(c3nc(OCC4(CN5CC[C@@H](F)C5)CC4)nc4c3CCN(c3cccc5cccc(Br)c35)C4)C1)N2]

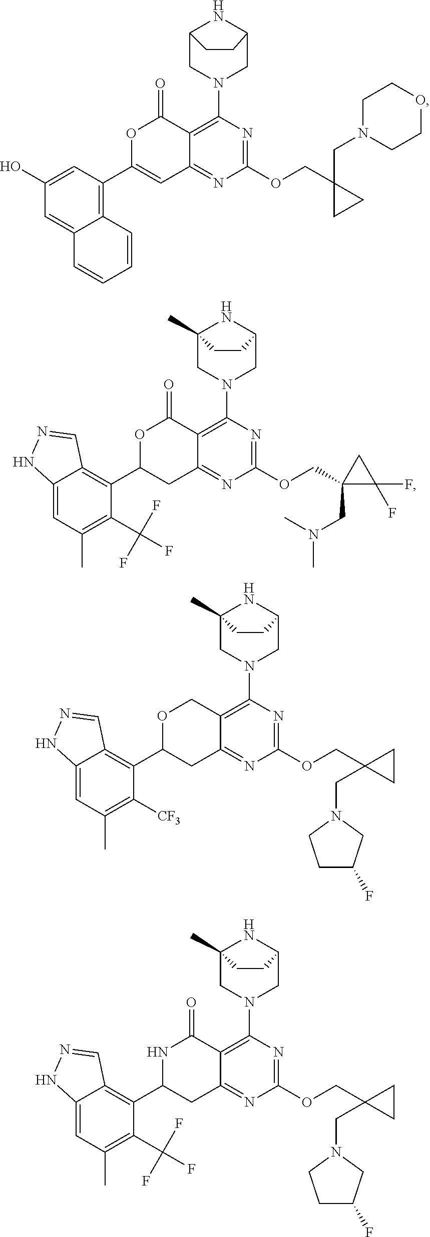 [SMILES: O=c1oc(-c2cc(O)cc3ccccc23)cc2nc(OCC3(CN4CCOCC4)CC3)nc(N3CC4CCC(C3)N4)c12 ; Cc1cc2[nH]ncc2c(C2Cc3nc(OC[C@]4(CN(C)C)CC4(F)F)nc(N4C[C@H]5CC[C@](C)(C4)N5)c3C(=O)O2)c1C(F)(F)F ; Cc1cc2[nH]ncc2c(C2Cc3nc(OCC4(CN5CC[C@@H](F)C5)CC4)nc(N4C[C@H]5CC[C@](C)(C4)N5)c3CO2)c1C(F)(F)F ; Cc1cc2[nH]ncc2c(C2Cc3nc(OCC4(CN5CC[C@@H](F)C5)CC4)nc(N4C[C@H]5CC[C@](C)(C4)N5)c3C(=O)N2)c1C(F)(F)F]

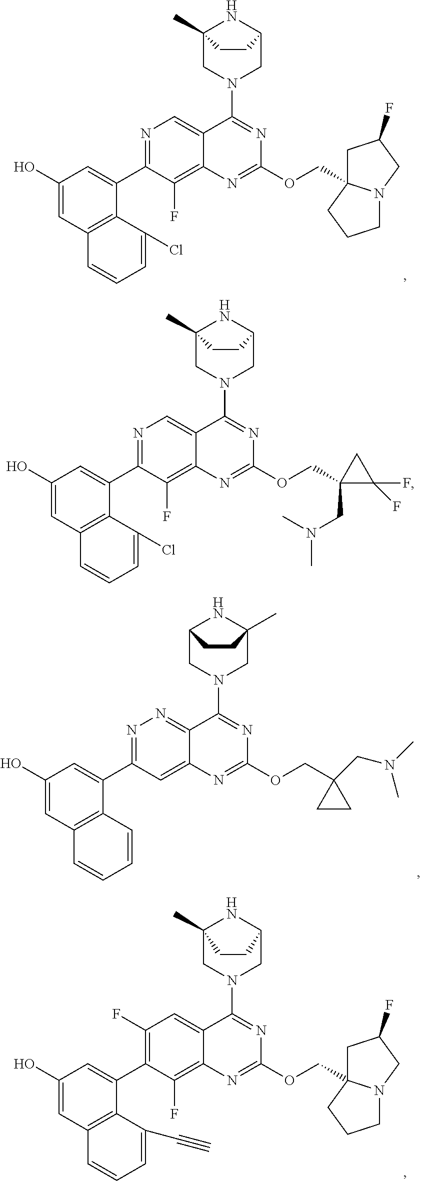

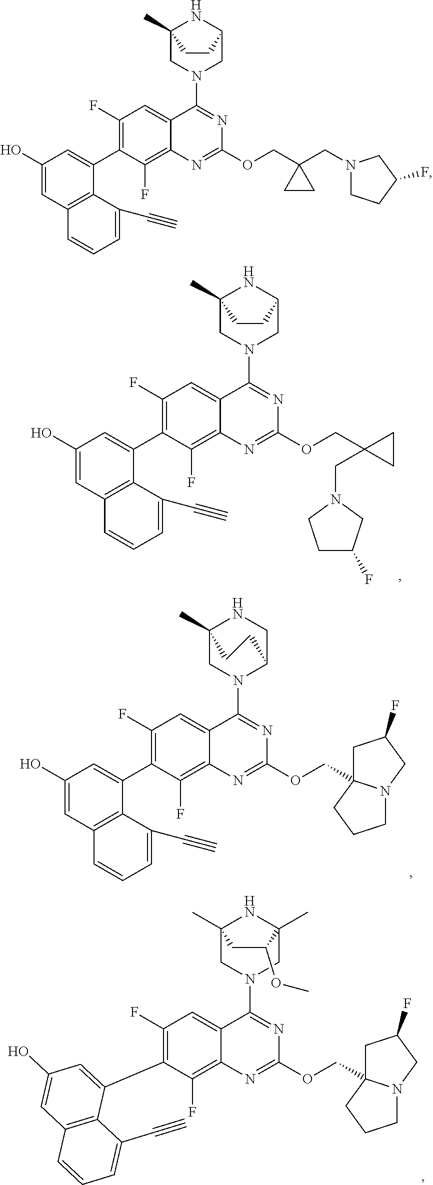

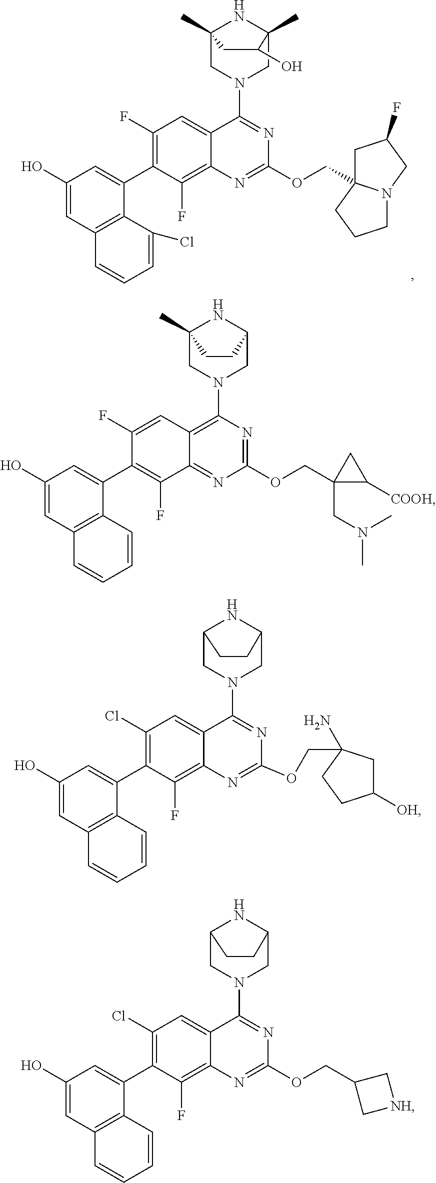

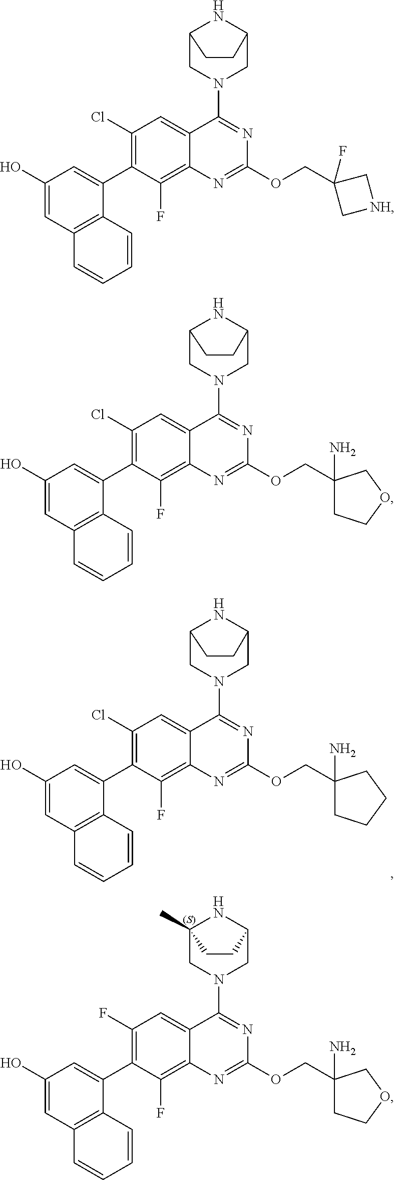

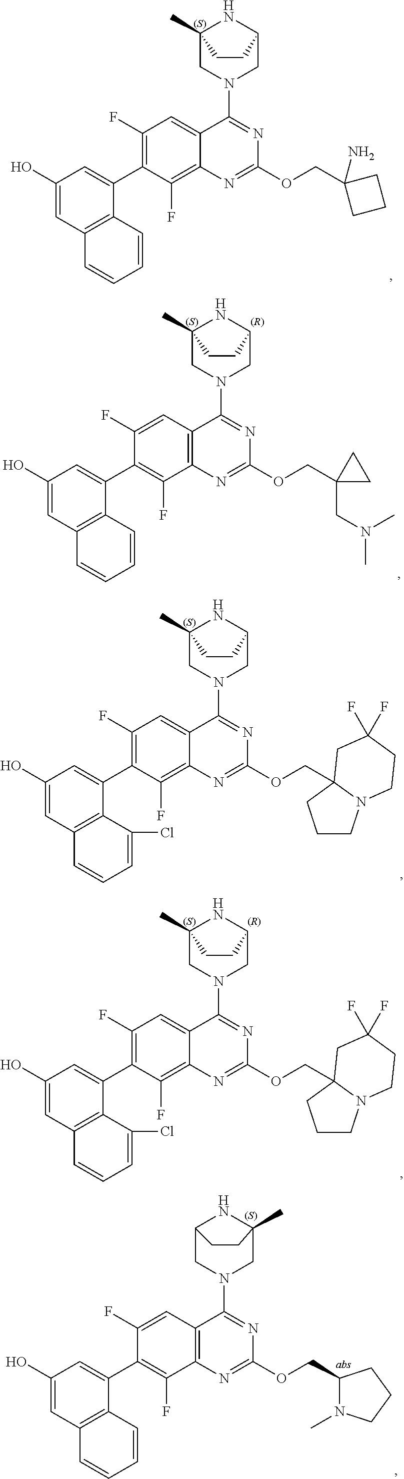

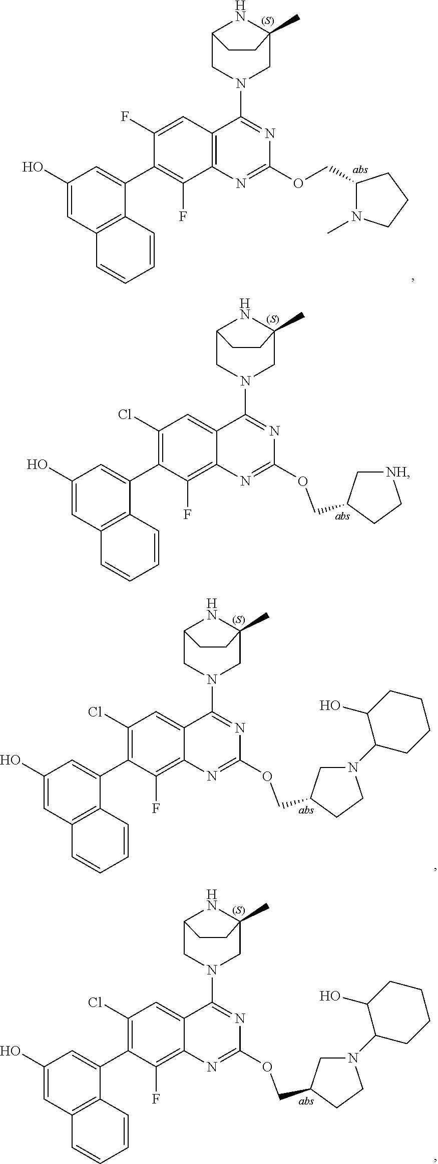

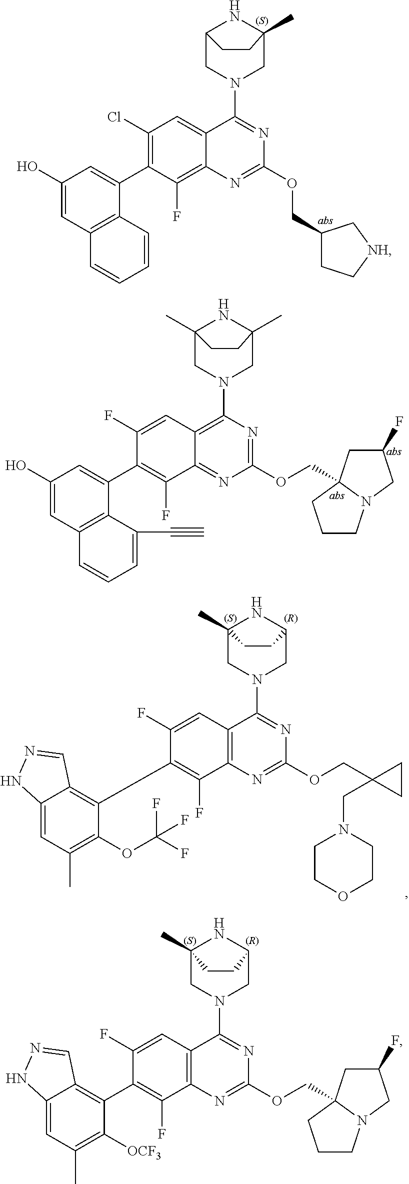

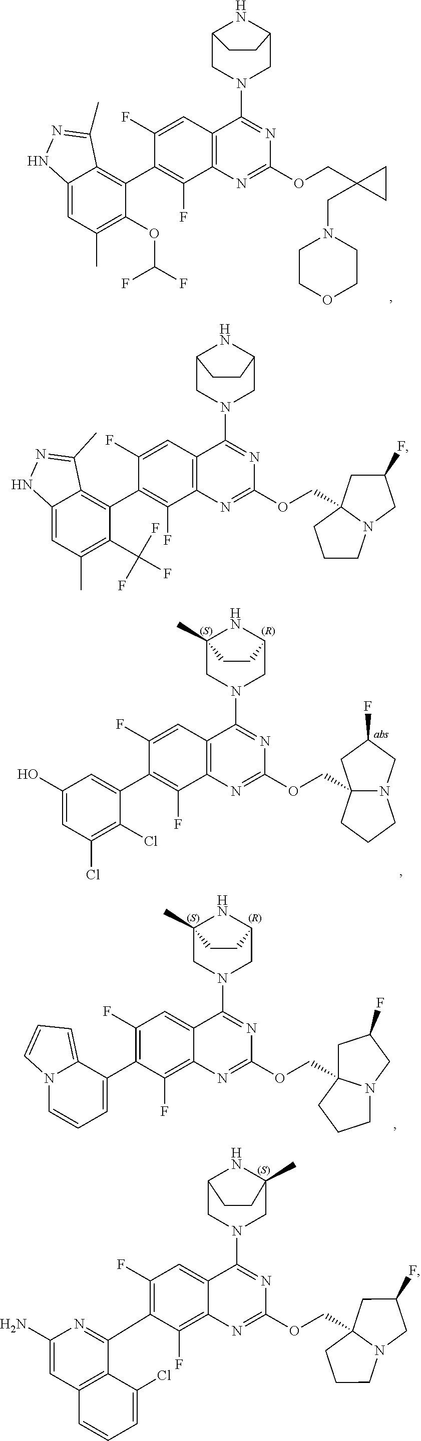

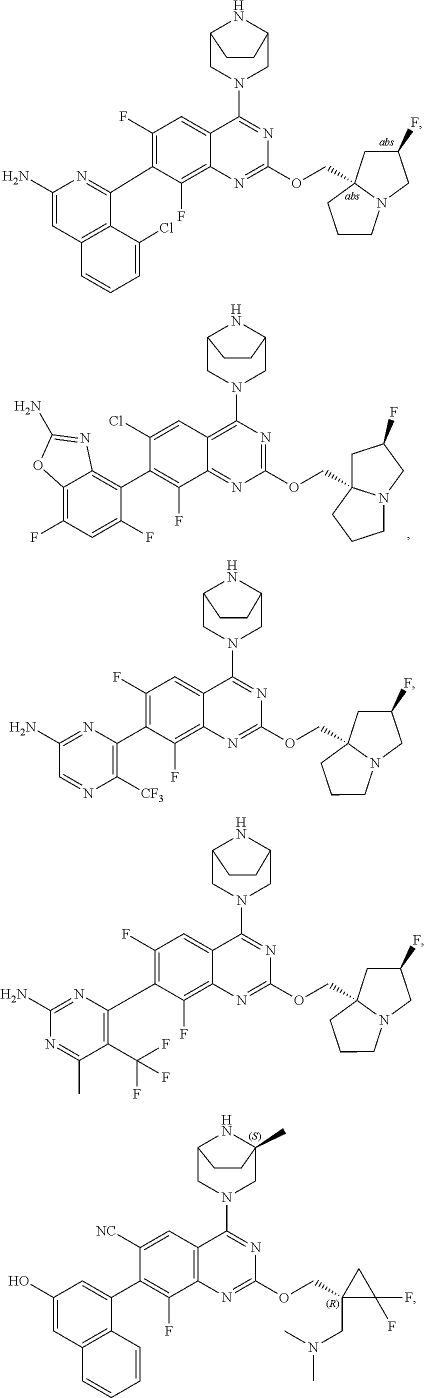

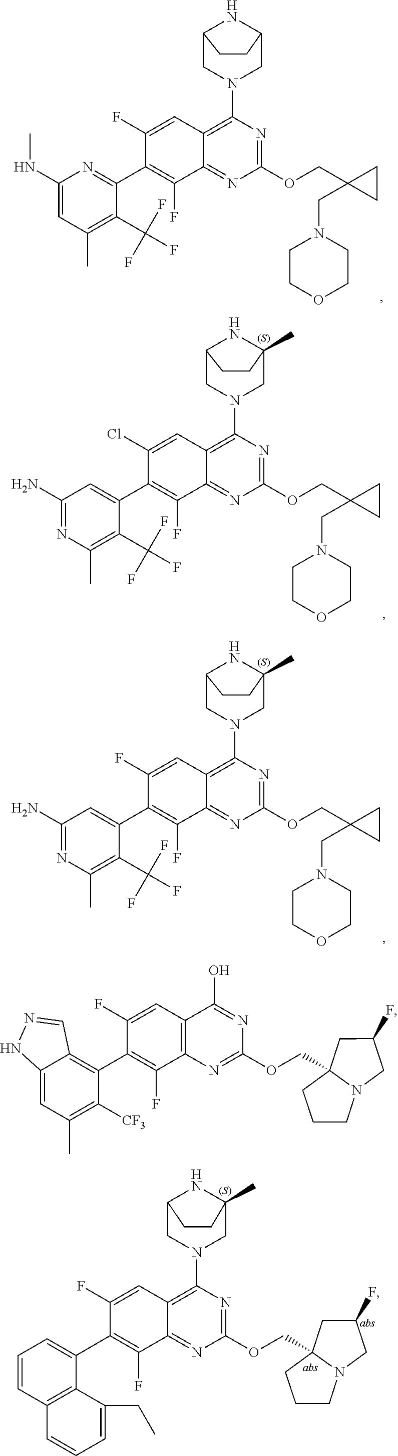

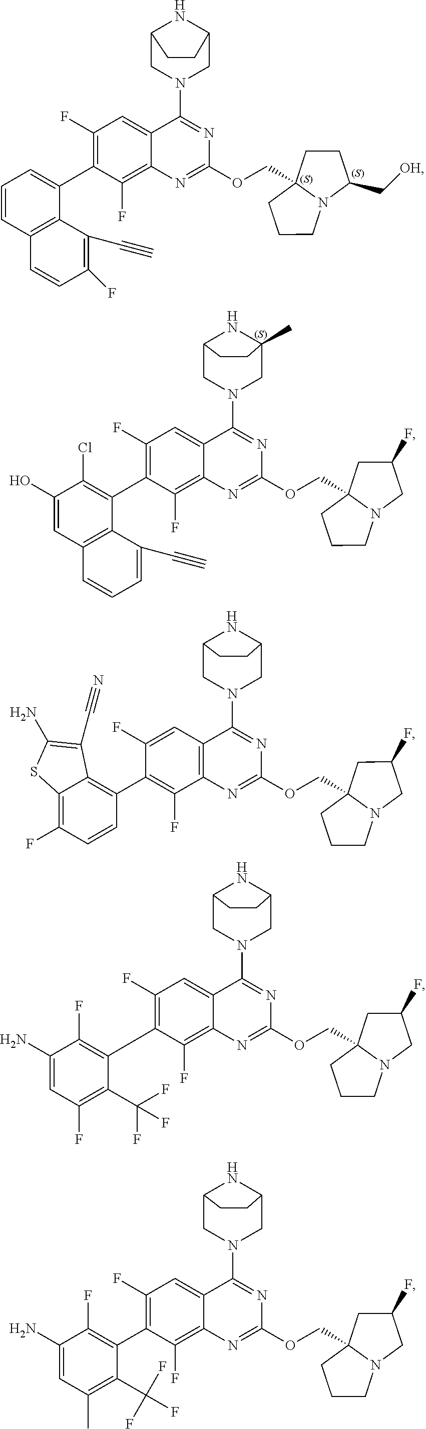

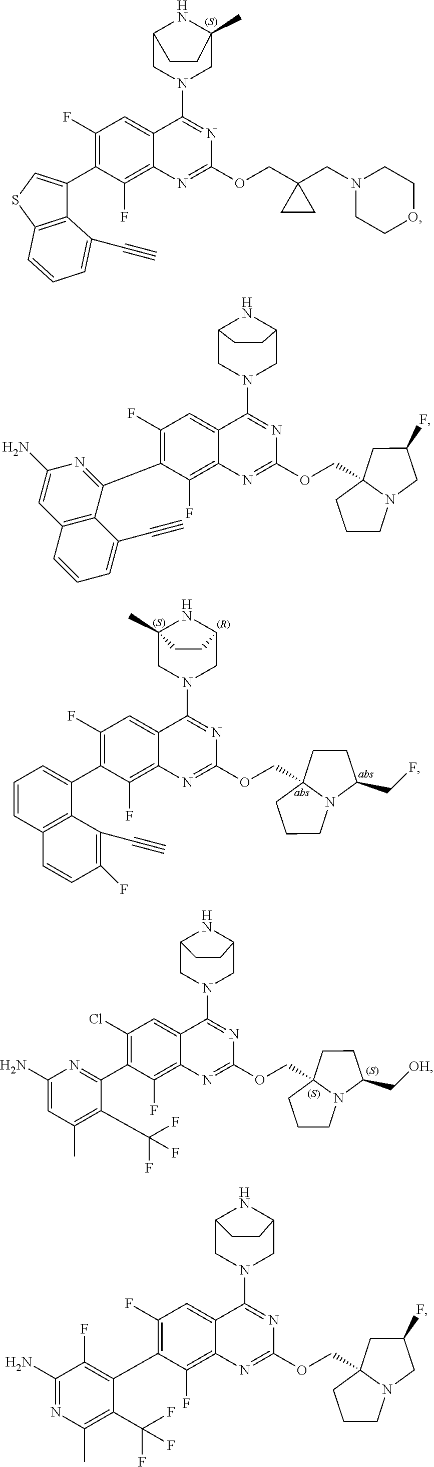

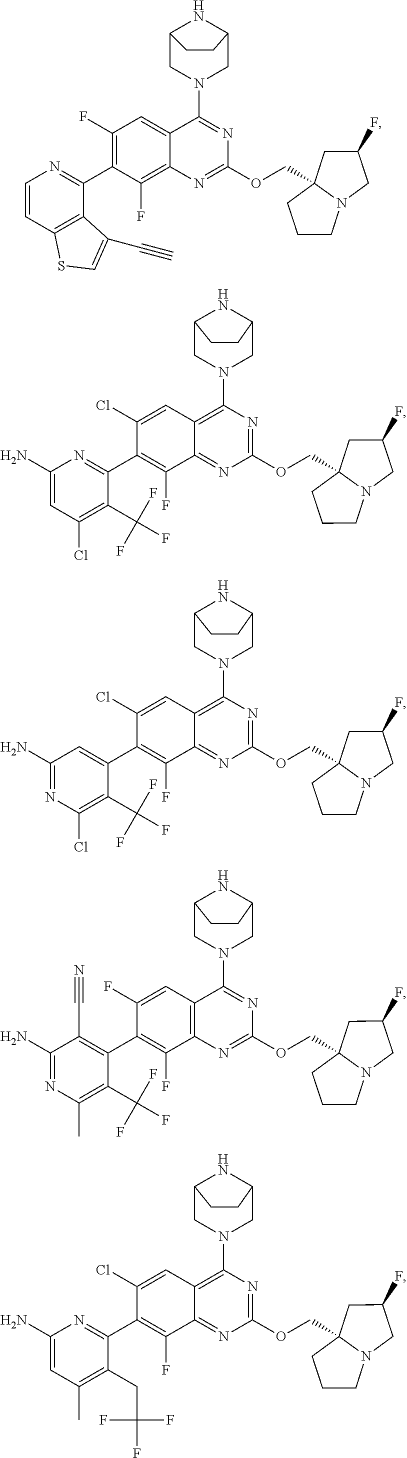 [SMILES: C#Cc1csc2ccnc(-c3c(F)cc4c(N5CC6CCC(C5)N6)nc(OC[C@@]56CCCN5C[C@H](F)C6)nc4c3F)c12 ; Nc1cc(Cl)c(C(F)(F)F)c(-c2c(Cl)cc3c(N4CC5CCC(C4)N5)nc(OC[C@@]45CCCN4C[C@H](F)C5)nc3c2F)n1 ; Nc1cc(-c2c(Cl)cc3c(N4CC5CCC(C4)N5)nc(OC[C@@]45CCCN4C[C@H](F)C5)nc3c2F)c(C(F)(F)F)c(Cl)n1 ; Cc1nc(N)c(C#N)c(-c2c(F)cc3c(N4CC5CCC(C4)N5)nc(OC[C@@]45CCCN4C[C@H](F)C5)nc3c2F)c1C(F)(F)F ; Cc1cc(N)nc(-c2c(Cl)cc3c(N4CC5CCC(C4)N5)nc(OC[C@@]45CCCN4C[C@H](F)C5)nc3c2F)c1CC(F)(F)F]

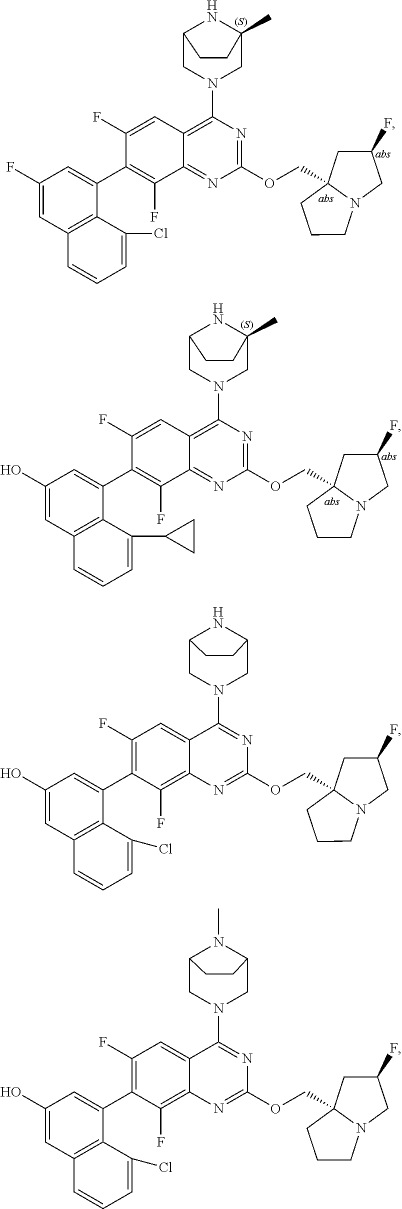

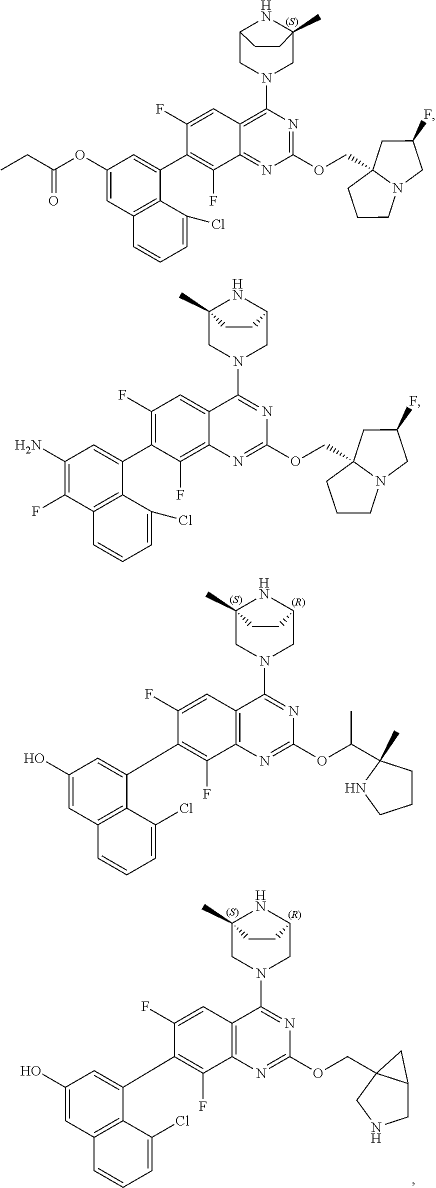

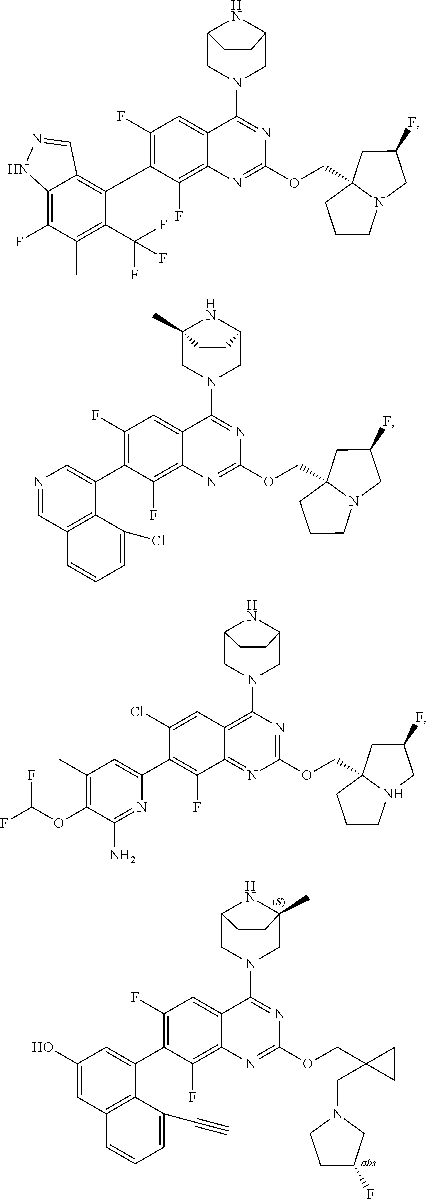

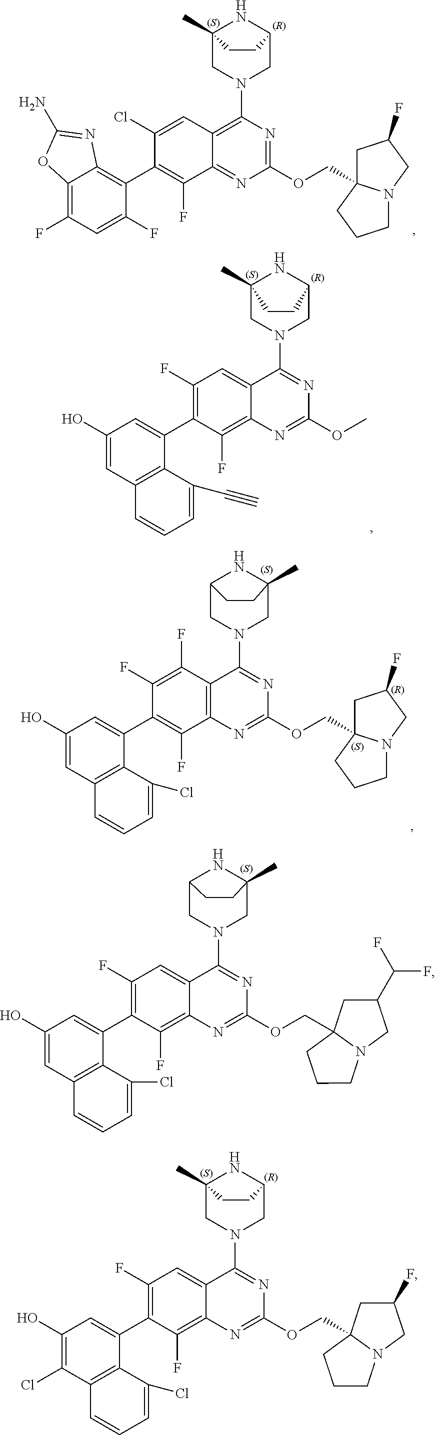

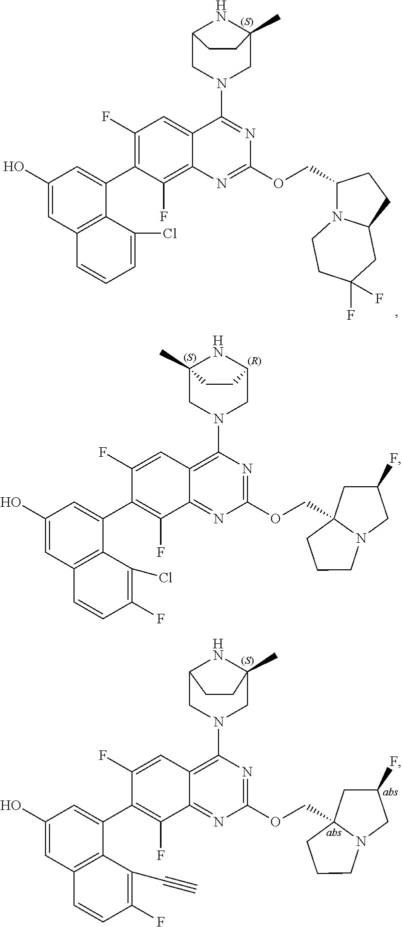

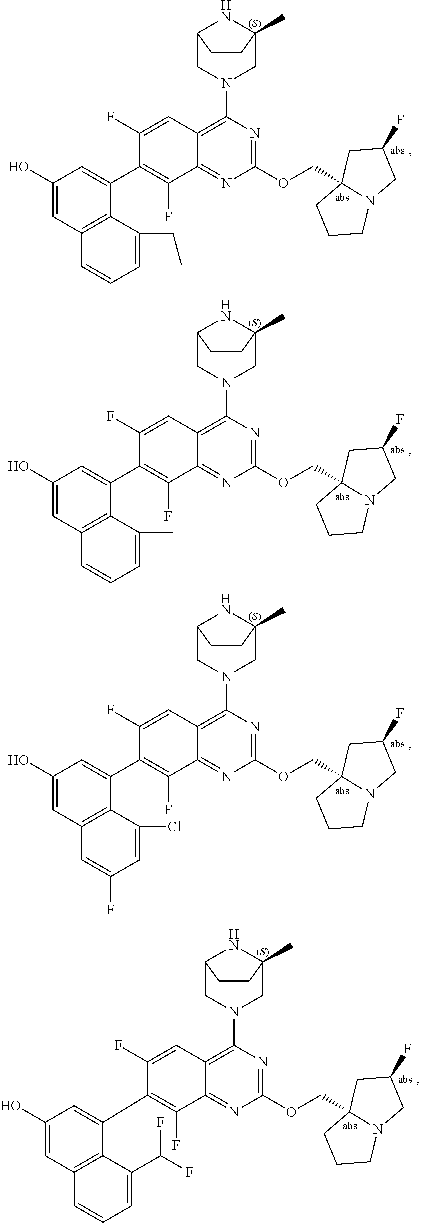

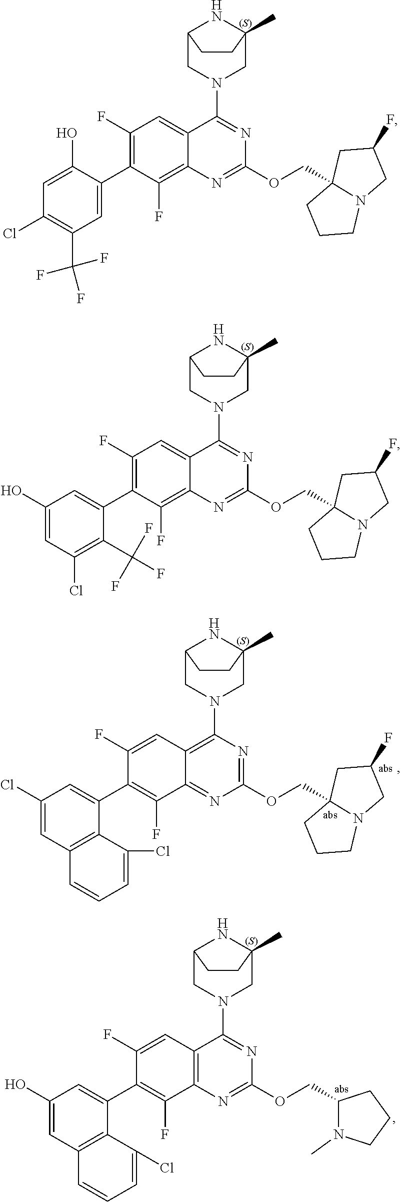

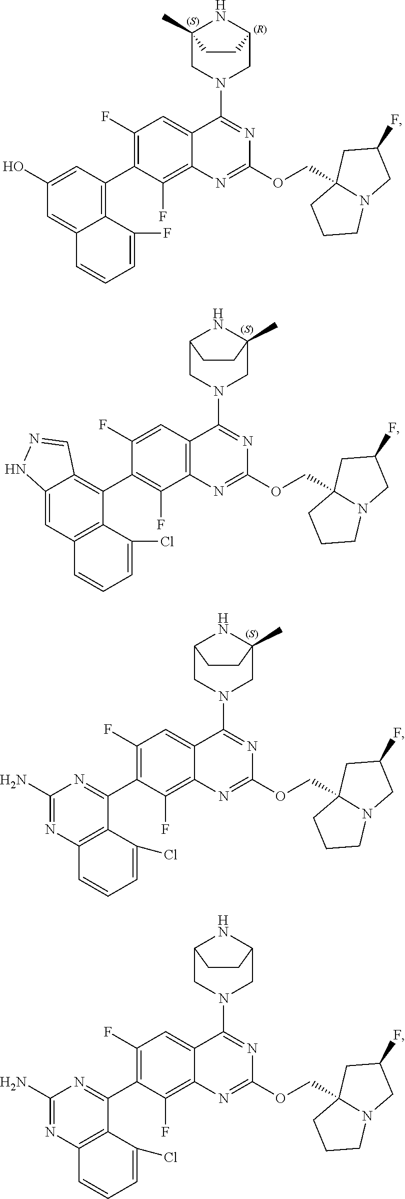

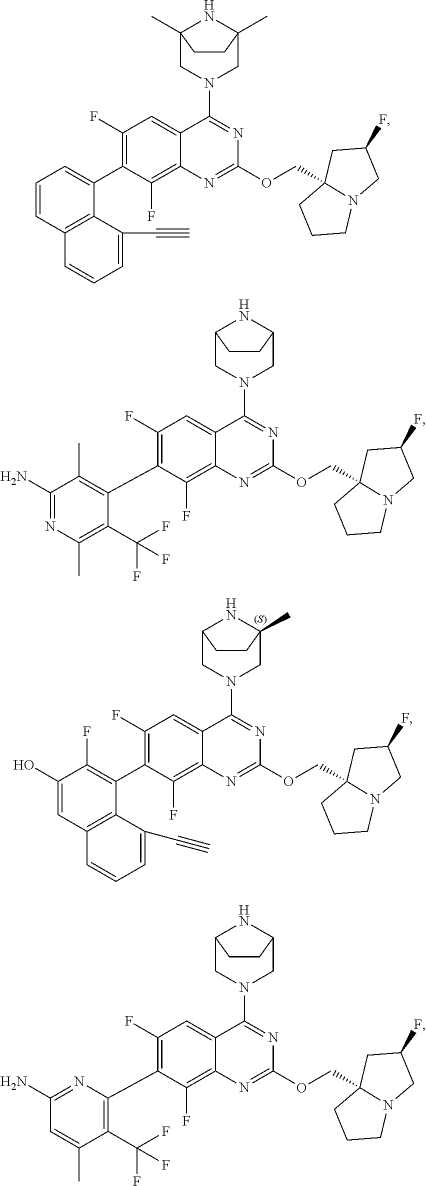

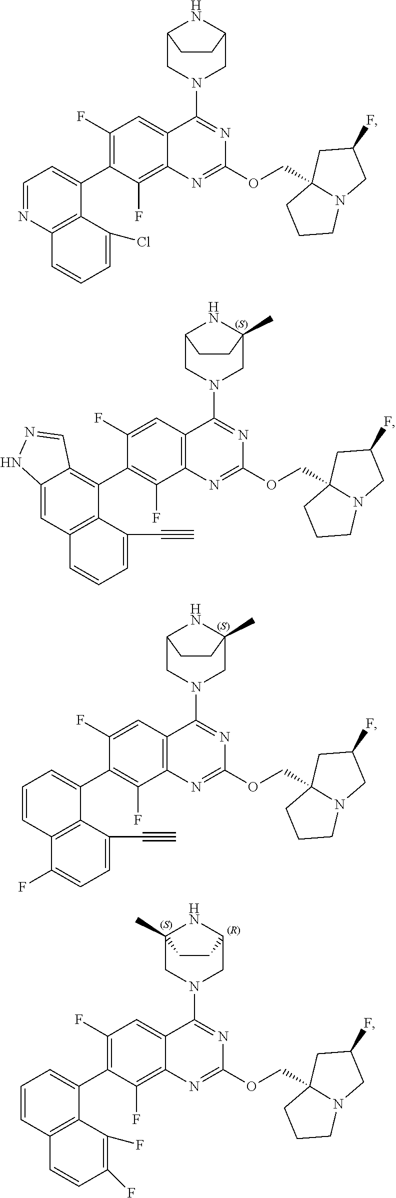

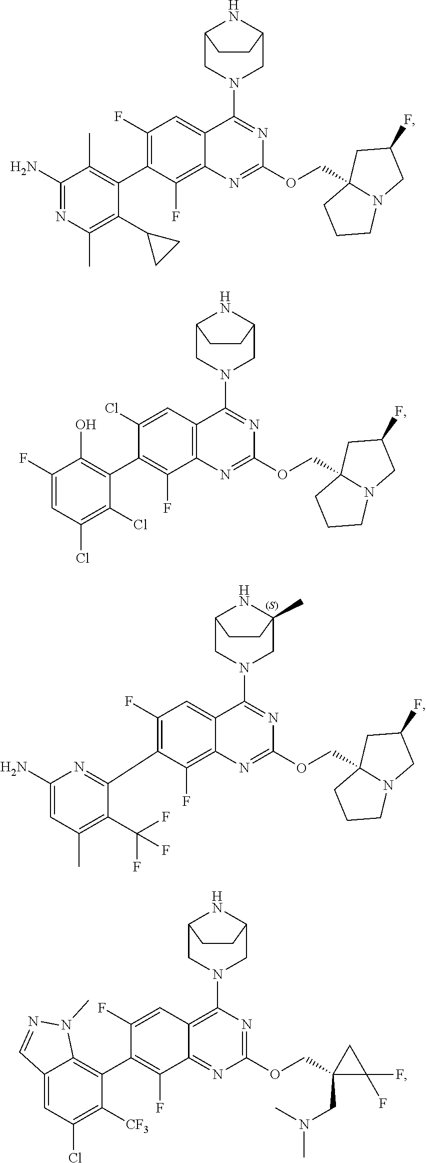

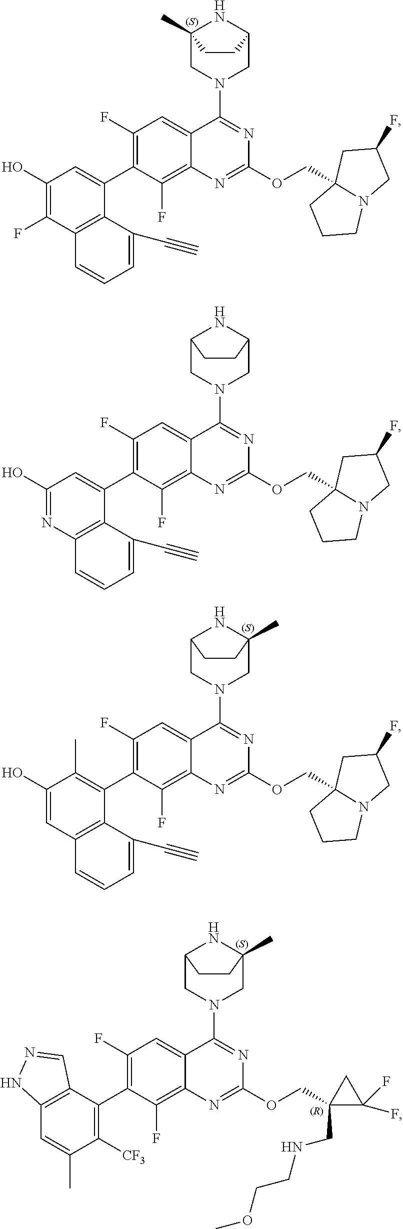

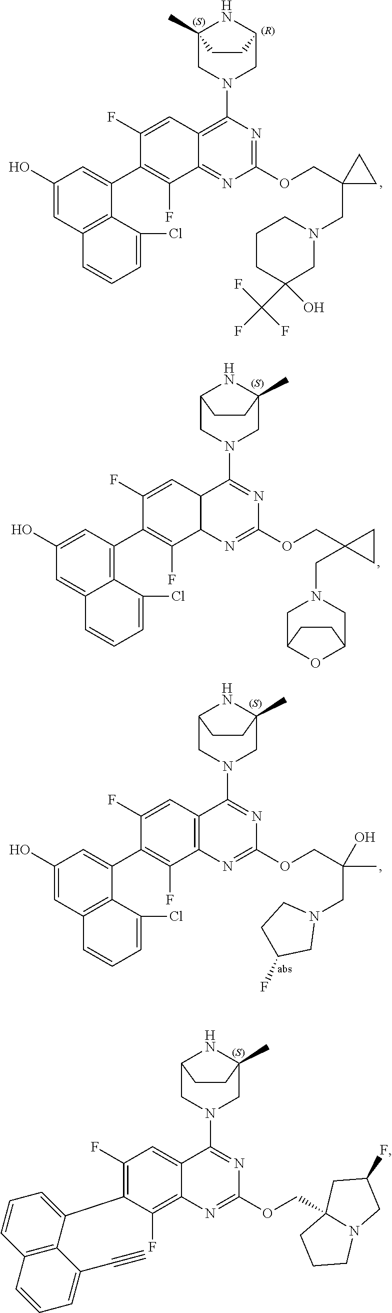

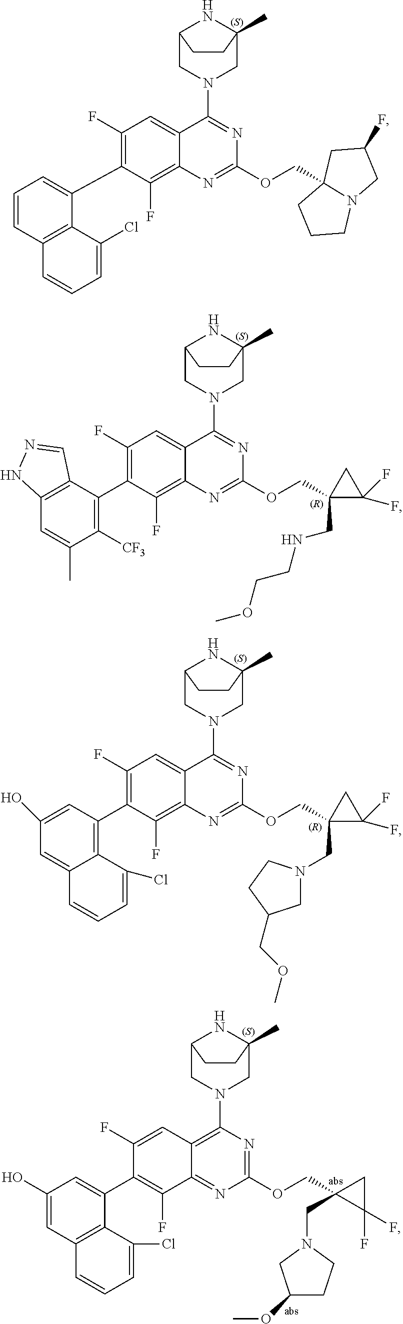

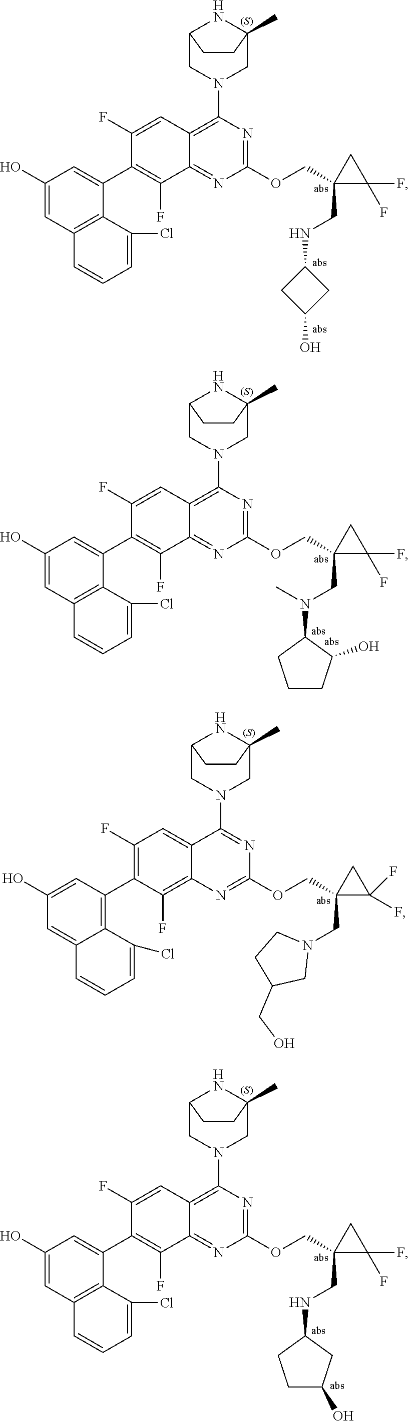

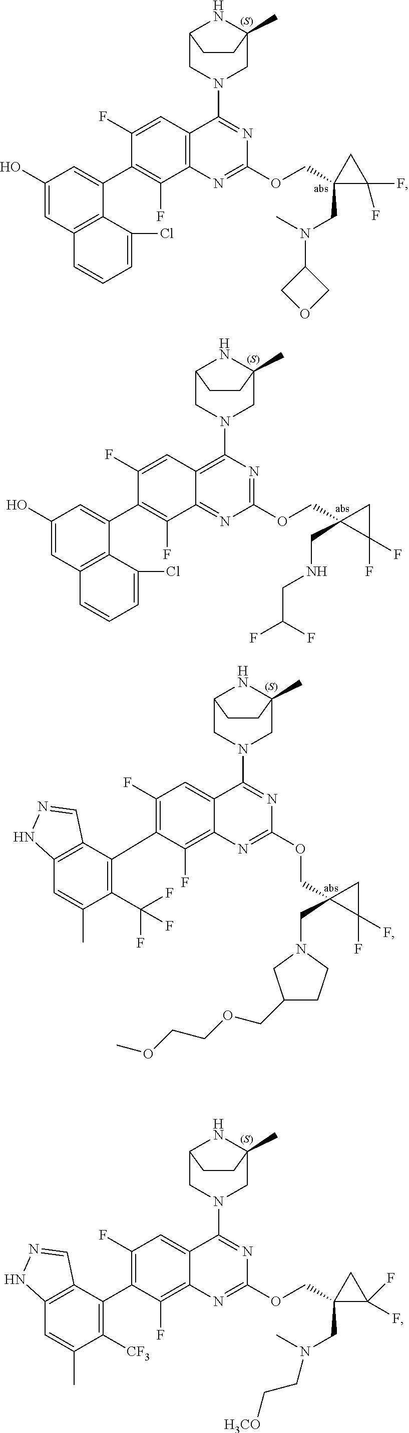

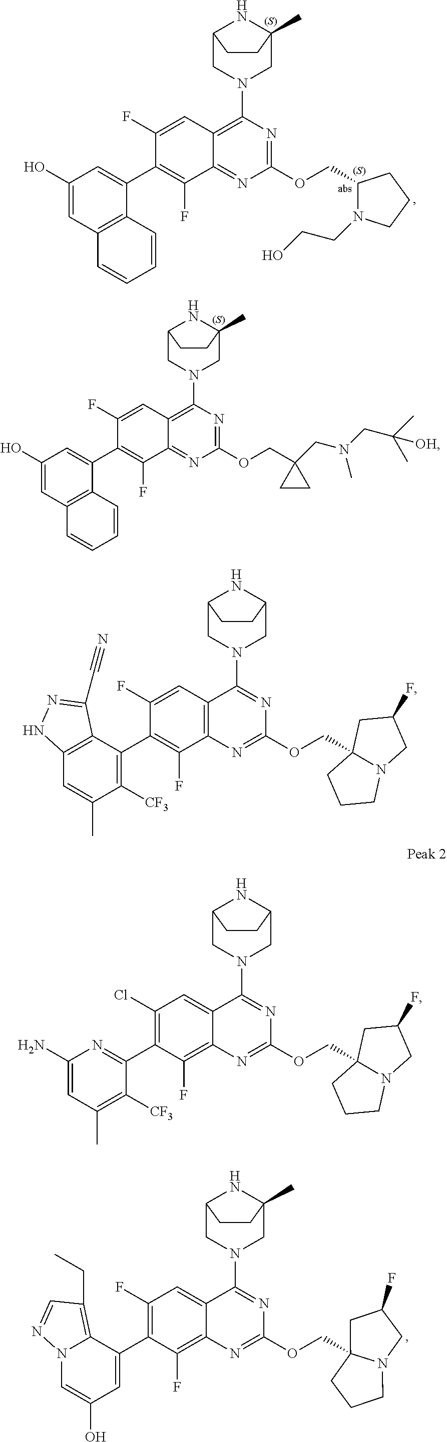

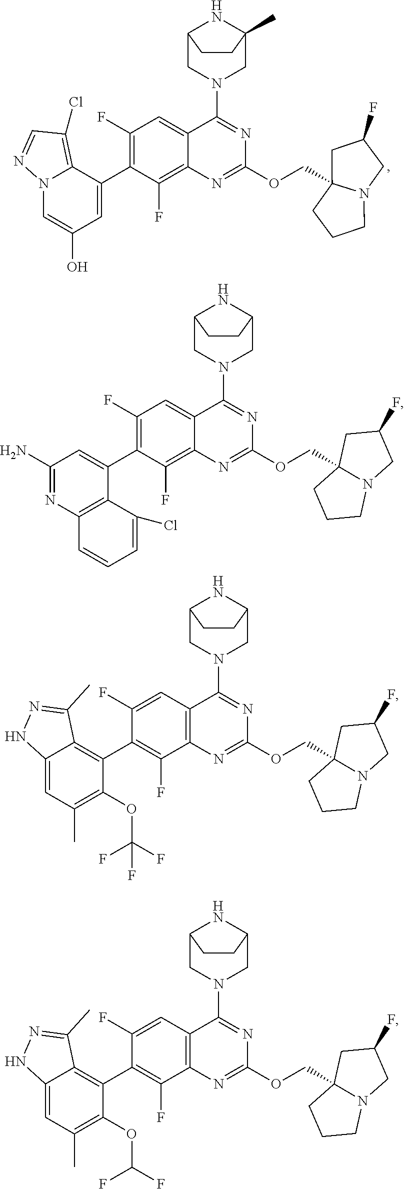 [SMILES: C[C@]12CCC(CN(c3nc(OC[C@@]45CCCN4C[C@H](F)C5)nc4c(F)c(-c5cc(O)cn6ncc(Cl)c56)c(F)cc34)C1)N2 ; Nc1cc(-c2c(F)cc3c(N4CC5CCC(C4)N5)nc(OC[C@@]45CCCN4C[C@H](F)C5)nc3c2F)c2c(Cl)cccc2n1 ; Cc1cc2[nH]nc(C)c2c(-c2c(F)cc3c(N4CC5CCC(C4)N5)nc(OC[C@@]45CCCN4C[C@H](F)C5)nc3c2F)c1OC(F)(F)F ; Cc1cc2[nH]nc(C)c2c(-c2c(F)cc3c(N4CC5CCC(C4)N5)nc(OC[C@@]45CCCN4C[C@H](F)C5)nc3c2F)c1OC(F)F]

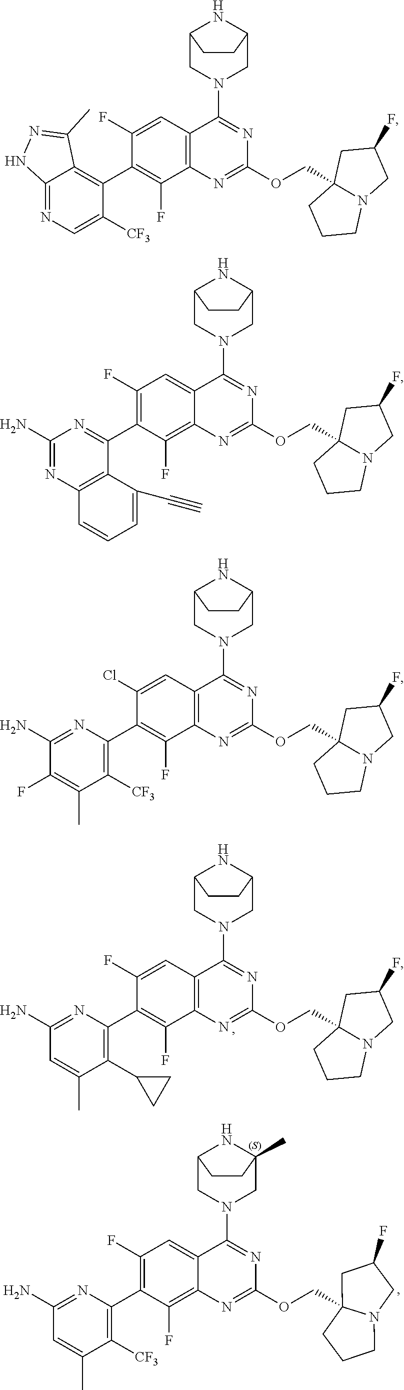

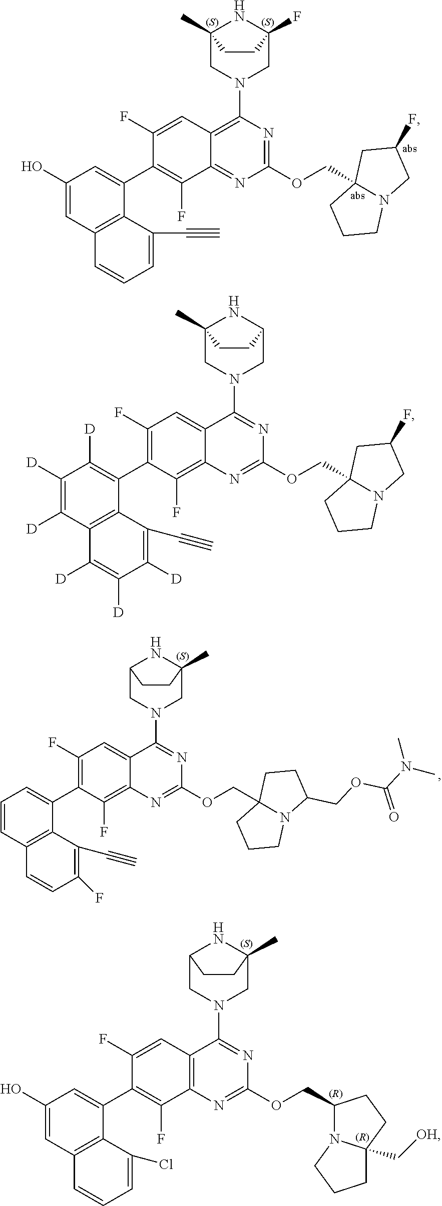

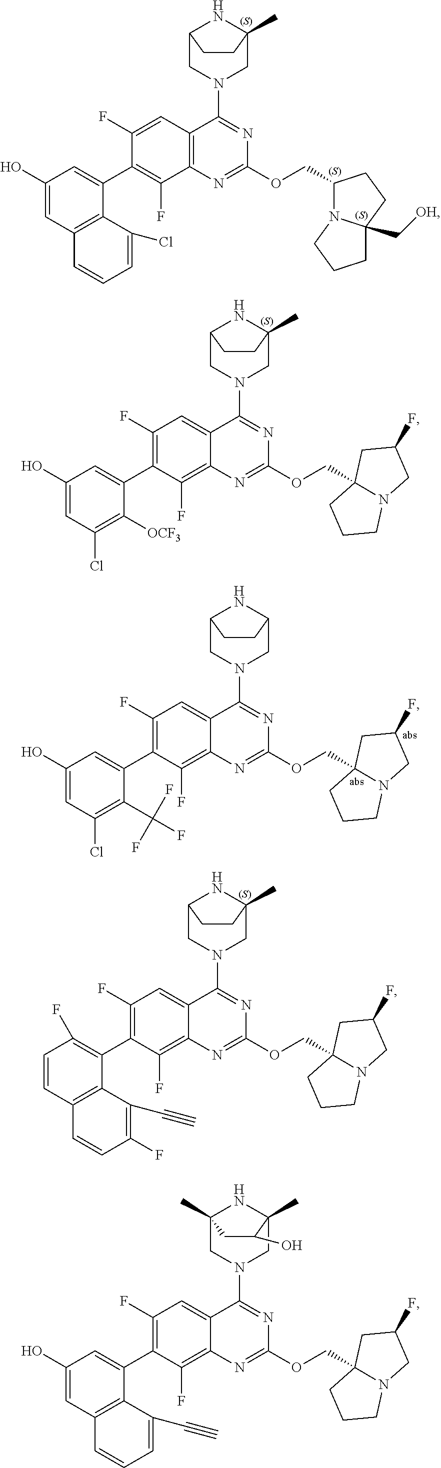

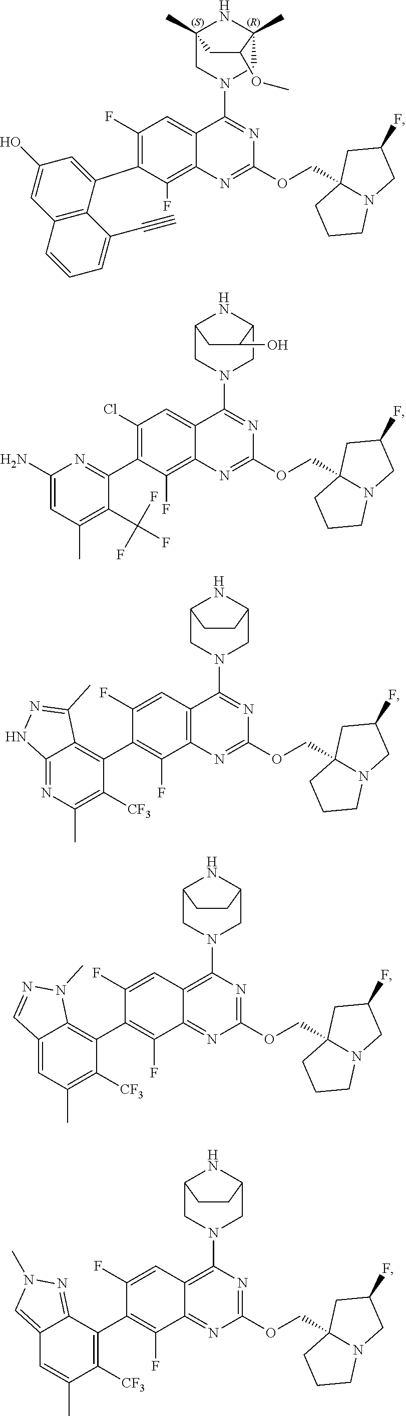

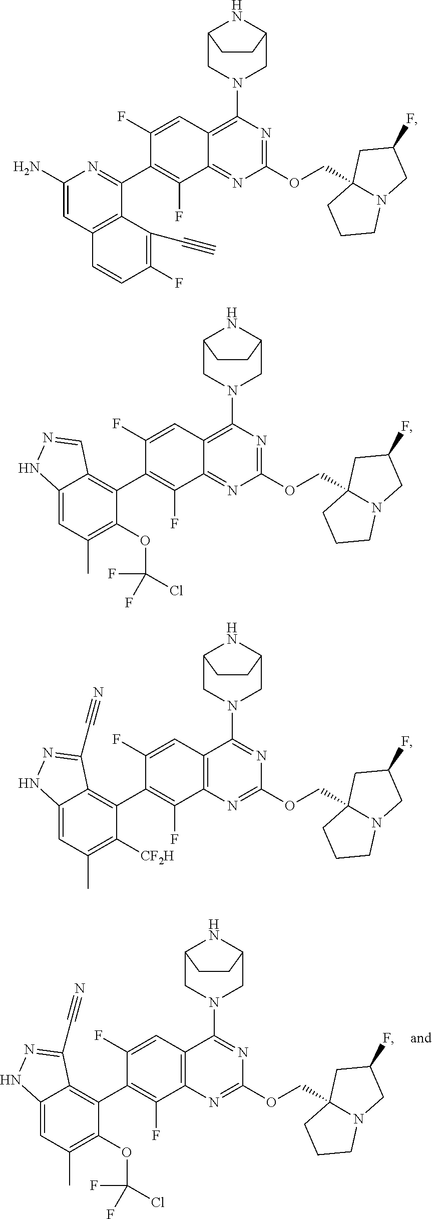 [SMILES: C#Cc1c(F)ccc2cc(N)nc(-c3c(F)cc4c(N5CC6CCC(C5)N6)nc(OC[C@@]56CCCN5C[C@H](F)C6)nc4c3F)c12 ; Cc1cc2[nH]ncc2c(-c2c(F)cc3c(N4CC5CCC(C4)N5)nc(OC[C@@]45CCCN4C[C@H](F)C5)nc3c2F)c1OC(F)(F)Cl ; Cc1cc2[nH]nc(C#N)c2c(-c2c(F)cc3c(N4CC5CCC(C4)N5)nc(OC[C@@]45CCCN4C[C@H](F)C5)nc3c2F)c1C(F)F ; Cc1cc2[nH]nc(C#N)c2c(-c2c(F)cc3c(N4CC5CCC(C4)N5)nc(OC[C@@]45CCCN4C[C@H](F)C5)nc3c2F)c1OC(F)(F)Cl ; "and"]

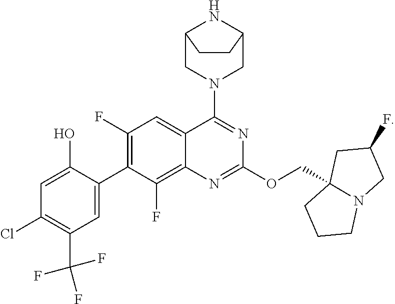 [SMILES: Oc1cc(Cl)c(C(F)(F)F)cc1-c1c(F)cc2c(N3CC4CCC(C3)N4)nc(OC[C@@]34CCCN3C[C@H](F)C4)nc2c1F]

In specific embodiments, the present disclosure provides a compound or a pharmaceutically acceptable salt thereof selected from the group consisting of:

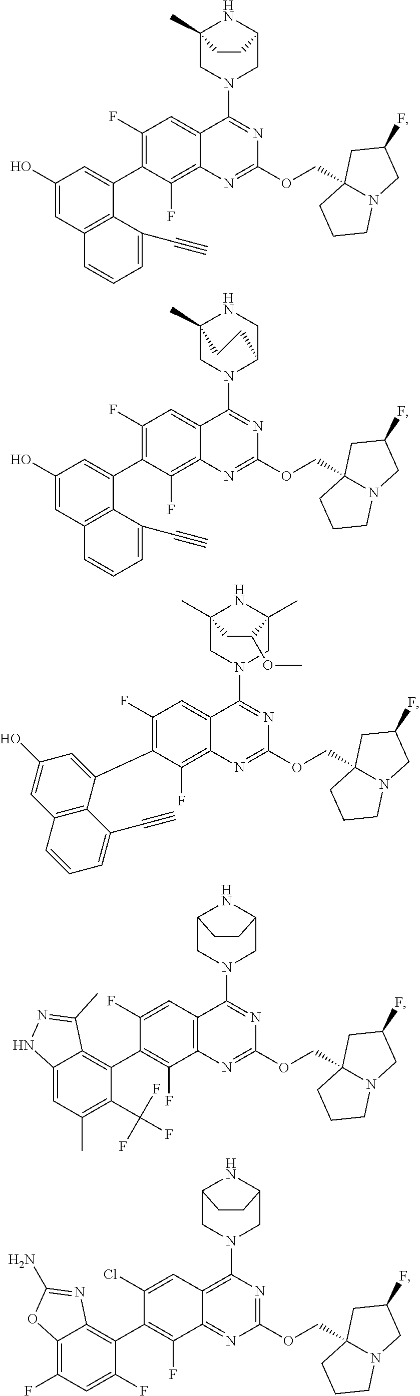 [SMILES: C#Cc1cccc2cc(O)cc(-c3c(F)cc4c(N5C[C@H]6CC[C@](C)(C5)N6)nc(OC[C@@]56CCCN5C[C@H](F)C6)nc4c3F)c12 ; C#Cc1cccc2cc(O)cc(-c3c(F)cc4c(N5C[C@@]6(C)CC[C@H]5CN6)nc(OC[C@@]56CCCN5C[C@H](F)C6)nc4c3F)c12 ; C#Cc1cccc2cc(O)cc(-c3c(F)cc4c(N5C[C@]6(C)CC(OC)[C@](C)(C5)N6)nc(OC[C@@]56CCCN5C[C@H](F)C6)nc4c3F)c12 ; Cc1cc2[nH]nc(C)c2c(-c2c(F)cc3c(N4CC5CCC(C4)N5)nc(OC[C@@]45CCCN4C[C@H](F)C5)nc3c2F)c1C(F)(F)F ; Nc1nc2c(-c3c(Cl)cc4c(N5CC6CCC(C5)N6)nc(OC[C@@]56CCCN5C[C@H](F)C6)nc4c3F)c(F)cc(F)c2o1]

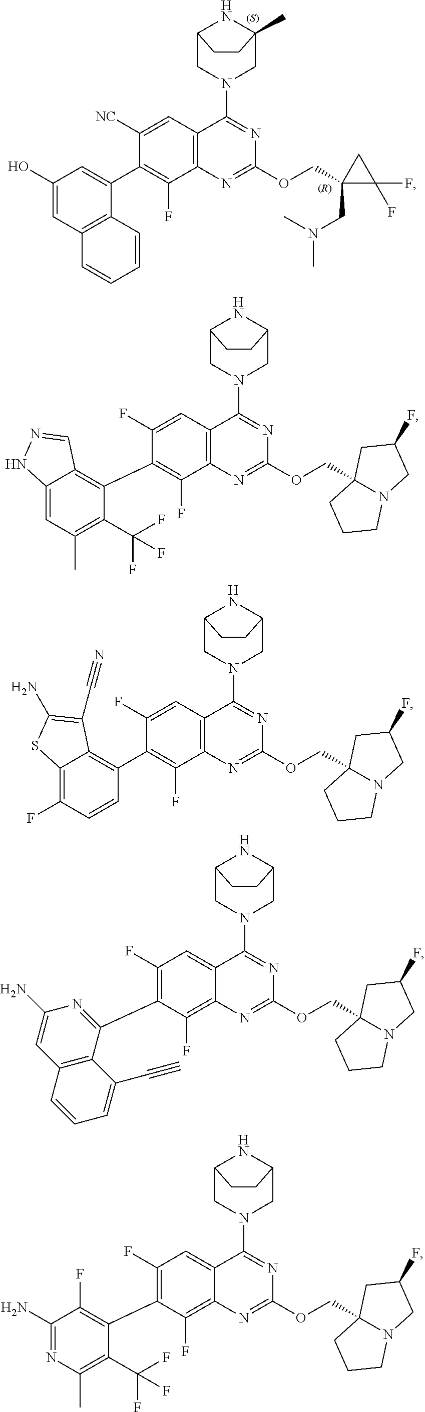

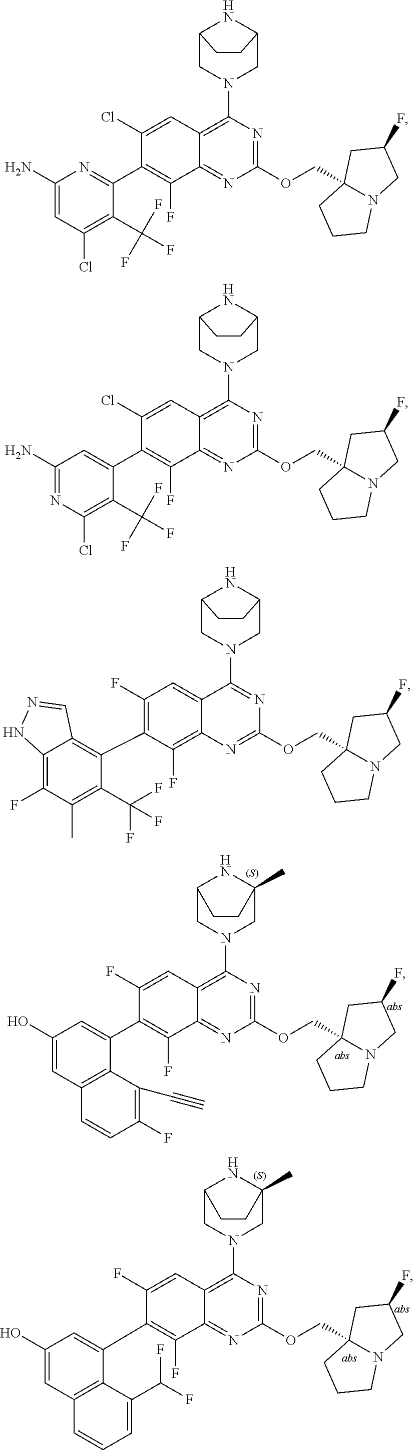

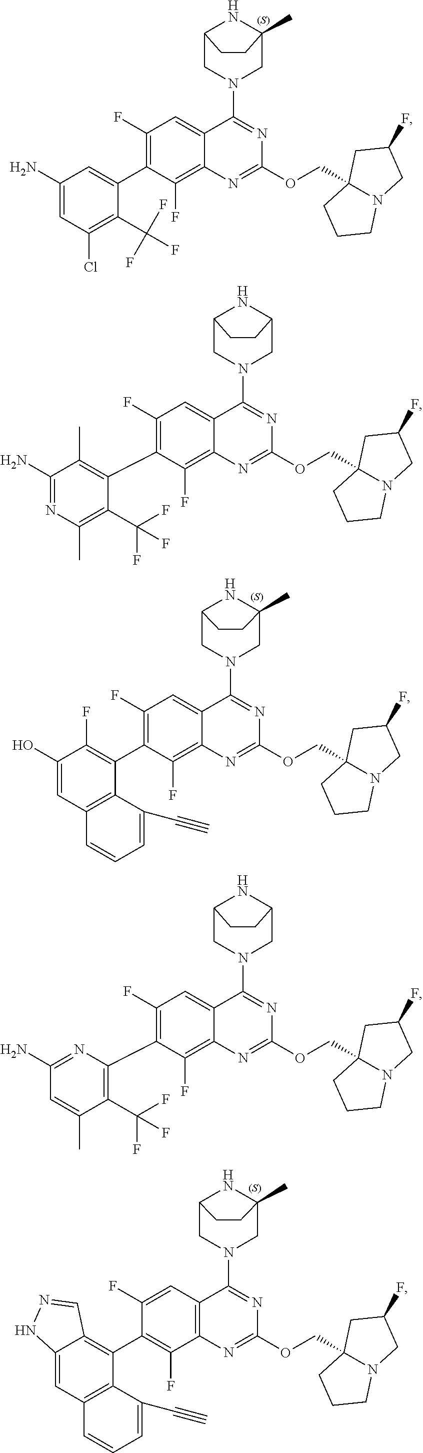

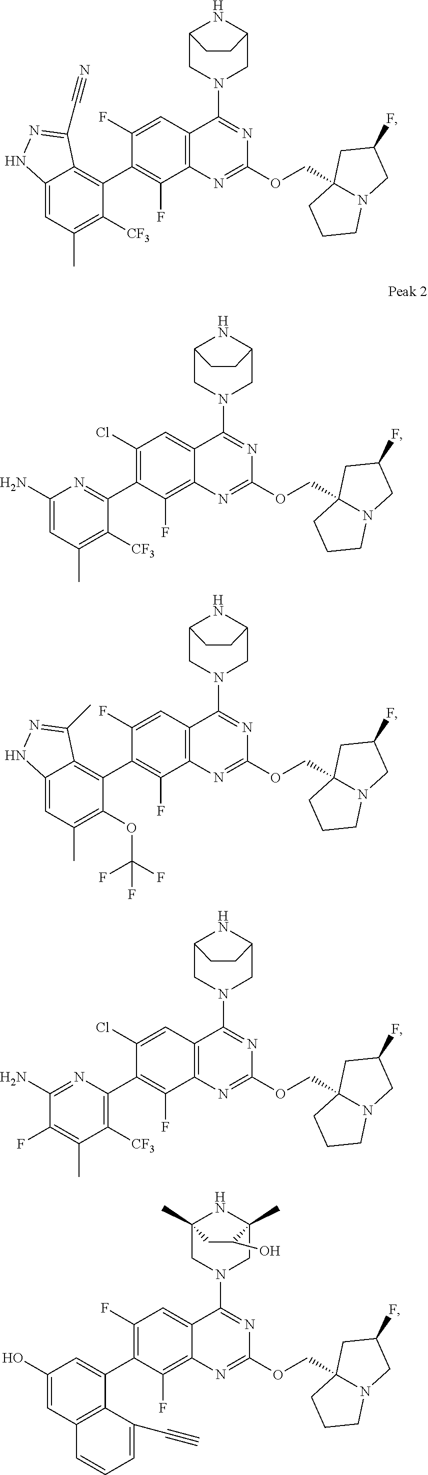 [SMILES: Cc1cc2[nH]nc(C#N)c2c(-c2c(F)cc3c(N4CC5CCC(C4)N5)nc(OC[C@@]45CCCN4C[C@H](F)C5)nc3c2F)c1C(F)(F)F ; Cc1cc(N)nc(-c2c(Cl)cc3c(N4CC5CCC(C4)N5)nc(OC[C@@]45CCCN4C[C@H](F)C5)nc3c2F)c1C(F)(F)F ; Cc1cc2[nH]nc(C)c2c(-c2c(F)cc3c(N4CC5CCC(C4)N5)nc(OC[C@@]45CCCN4C[C@H](F)C5)nc3c2F)c1OC(F)(F)F ; Cc1c(F)c(N)nc(-c2c(Cl)cc3c(N4CC5CCC(C4)N5)nc(OC[C@@]45CCCN4C[C@H](F)C5)nc3c2F)c1C(F)(F)F ; C#Cc1cccc2cc(O)cc(-c3c(F)cc4c(N5C[C@]6(C)CC(O)[C@@](C)(C5)N6)nc(OC[C@@]56CCCN5C[C@H](F)C6)nc4c3F)c12]

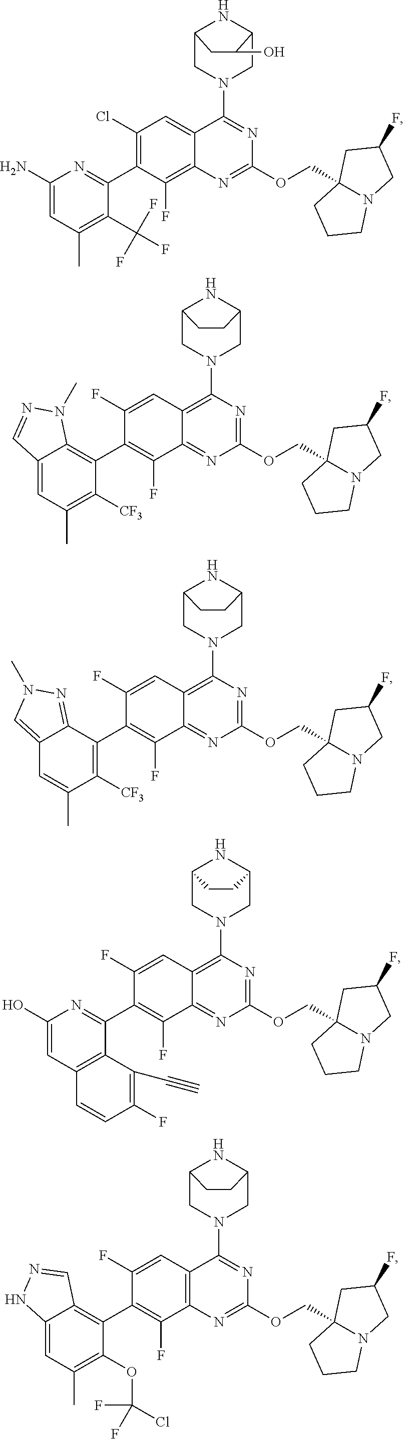 [SMILES: Cc1cc(N)nc(-c2c(Cl)cc3c(N4CC5CC(O)C(C4)N5)nc(OC[C@@]45CCCN4C[C@H](F)C5)nc3c2F)c1C(F)(F)F ; Cc1cc2cnn(C)c2c(-c2c(F)cc3c(N4CC5CCC(C4)N5)nc(OC[C@@]45CCCN4C[C@H](F)C5)nc3c2F)c1C(F)(F)F ; Cc1cc2cn(C)nc2c(-c2c(F)cc3c(N4CC5CCC(C4)N5)nc(OC[C@@]45CCCN4C[C@H](F)C5)nc3c2F)c1C(F)(F)F ; C#Cc1c(F)ccc2cc(O)nc(-c3c(F)cc4c(N5C[C@H]6CC[C@@H](C5)N6)nc(OC[C@@]56CCCN5C[C@H](F)C6)nc4c3F)c12 ; Cc1cc2[nH]ncc2c(-c2c(F)cc3c(N4CC5CCC(C4)N5)nc(OC[C@@]45CCCN4C[C@H](F)C5)nc3c2F)c1OC(F)(F)Cl]

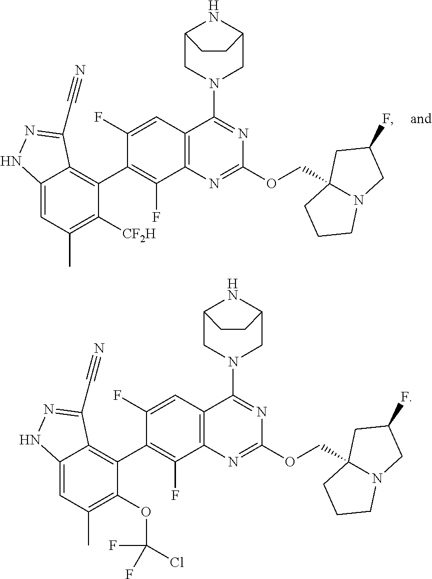 [SMILES: Cc1cc2[nH]nc(C#N)c2c(-c2c(F)cc3c(N4CC5CCC(C4)N5)nc(OC[C@@]45CCCN4C[C@H](F)C5)nc3c2F)c1C(F)F ; Cc1cc2[nH]nc(C#N)c2c(-c2c(F)cc3c(N4CC5CCC(C4)N5)nc(OC[C@@]45CCCN4C[C@H](F)C5)nc3c2F)c1OC(F)(F)Cl]

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “a compound of the disclosure” is to be understood to include “a compound of the disclosure or a pharmaceutically acceptable salt thereof”. Likewise “a compound of the instant disclosure”. “a compound of the disclosure”, “a compound of this disclosure”, and “a compound described herein” are used interchangeably and include both the compound, as well as a pharmaceutically acceptable salt thereof: “compounds of Formula (I)” are such compounds.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Alkoxyalkyl” means an alkoxy-alkyl group in which the alkoxy and alkyl groups are as previously defined. The bond to the parent moiety is through a carbon atom of the alkyl component. Non-limiting examples of suitable alkyoxyalkyl groups include methoxymethyl and methoxyethyl.

“Alkenyl” means an aliphatic hydrocarbon group containing at least one carbon-carbon double bond and which may be straight or branched. Branched means that one or more alkyl groups such as methyl, ethyl or propyl, are attached to a linear alkenyl chain. Non-limiting examples of alkenyl groups include ethenyl, propenyl, n-butenyl, 3-methylbut-2-enyl, and n-pentenyl.

“Alkylcarbonylalkyl” means an alkyl-C(O)-alkyl group. The bond to the parent group is through the carbon atom of an alkyl component.

“Alkynyl” means an aliphatic hydrocarbon group containing at least one carbon-carbon triple bond and which may be straight or branched. Non-limiting examples include ethynyl, propynyl, and butynyl.

“Aminoalkyl” means -alkyl-NH₂ group in which the alkyl is as previously defined. The bond to the parent moiety is through a carbon atom of the alkyl component. Non-limiting examples of suitable aminoalkyl groups include aminomethyl and aminoethyl. “Alkylamino” means —NH-alkyl group in which the alkyl is as previously defined. The bond to the parent moiety is through the nitrogen of the amino component.

“Alkylthio” means an alkyl-S— group in which the alkyl group is as previously described. Non-limiting examples of suitable alkylthio groups include methylthio and ethylthio. The bond to the parent moiety is through the sulfur.

“Alkylaminoalkyl” means an alkylamino as previously defined, wherein the amino atom is substituted by one alkyl substituent. Non-limiting examples of suitable alkylaminoalkyl groups include methylaminomethyl [(CH₃)(H)NCH₂—] and N-propyl-aminoethyl [(CH₃CH₂CH₂)(H)N—CH₂CH₂—].

“Bicyclic ring system” refers to two joined rings. The rings may be fused, i.e., share two adjacent atoms, or “spirocyclic”, i.e., share only a single atom.

“Carbamoyl” means a H₂N—C(O)— group, which is the univalent group formed by loss of —OH group of carbamic acid. The bond to the parent group is through the carbon atom of the carbonyl component.

“Cyanoalkyl” means an -alkyl-CN group in which the alkyl is as previously defined. The bond to the parent moiety is through a carbon atom of the alkyl component. Non-limiting examples of suitable cyanoalkyl groups include cyanomethyl and 3-cyanopropyl.

“Cycloalkyl” means a saturated cyclic hydrocarbon radical. In particular embodiments, the cycloalkyl group has 3-12 carbon atoms, forming 1-3 carbocyclic rings that are fused. Examples of cycloalkyl include cyclopropyl, cyclobutyl, cyclopentyl, cyclohexyl, cycloheptyl, adamantyl, and the like.

“Dialkylamino” means an alkylamino as previously defined, wherein the amino atom is substituted by two alkyl substituents, e.g., —N(CH₃)2.

“Dialkylaminoalkyl” means an aminoalkyl as previously defined, wherein the amino atom is also substituted by two alkyl substituents. The alkyl groups substituted on the amino atom can be the same or different. Non-limiting examples of suitable dialkylaminoalkyl groups include dimethyl aminomethyl [(CH₃)₂NCH₂-] and N-ethyl-N-methylaminoethyl [(CH₃CH₂)(CH₃)N—CH₂CH₂—].

“Fluoroalkyl” includes mono-substituted as well as multiple fluoro-substituted alkyl groups, up to perfluoro substituted alkyl. For example, fluoromethyl, 1.1-difluoroethyl, trifluoromethyl or 1.1,1,2,2-pentafluorobutyl are included.

“Fluoroalkylthio” includes mono-substituted as well as multiple fluoro-substituted alkylthio groups. For example, fluoromethylthio, 2.2-difluoroethylthio, and trifluoromethylthio are included.

“Fluoroalkoxy” includes mono-substituted as well as multiple fluoro-substituted alkoxy groups. For example, fluoromethoxy, 2.2-difluoroethoxy, and trifluoromethoxy are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heterocycloalkyl” means a non-aromatic monocyclic, bicyclic or tricyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. In some embodiments, heterocycloalkyls contain about 5 to about 6 ring atoms. The prefix aza, oxa or thia before the heterocyclyl root name means that at least a nitrogen, oxygen or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1.4-dioxanyl, tetrahydrofuranyl, tetrahydrothiophenyl, and the like.

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaromatic groups include pyridinyl, pyrimidinyl, pyrrolyl, pyridazinyl, isoxazolyl, thiazolyl, oxazolyl, indolyl, benzoxazolyl, benzothiazolyl, and imidazolyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Spiroheterocycloalkyl” refers to two joined rings, wherein the rings share only a single atom, and at least one of the rings is a heterocycloalkyl group.

“Trialkylsilyl” means a group having three alkyl groups, as previously defined, bonded to a silicon atom, which is in turn bonded to the parent group. The alkyl groups can be the same or different. Non-limiting examples of trialkylsilyl groups include trimethylsilyl, diethyl methyl silyl, and tri-n-butyl silyl.

“Tricyclic ring system” refers to three joined rings. The rings may be fused, i.e., share adjacent atoms or “spirocyclic”, i.e., share only a single atom.

When any variable (e.g., R^{A} occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{A}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{B}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

Compounds of the disclosure may contain one or more asymmetric centers and can thus occur as racemates and racemic mixtures, single enantiomers, diastereoisomeric mixtures and individual diastereoisomers. Centers of asymmetry that are present in the compounds of the disclosure can all independently of one another have S configuration or R configuration. The compounds of this disclosure include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of this disclosure. Where a structural formula or chemical name specifies a particular configuration at a stereocenter, the enantiomer or stereoisomer of the compound resulting from that specified stereocenter is intended. Where a structural formula of the compounds of this disclosure indicates a straight line at a chiral center, the structural formula includes both the S and R stereoisomers associated with the chiral center and mixtures thereof.

Compounds of the disclosure may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of a compound of the disclosure may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

For compounds of the disclosure described herein which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present disclosure.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of the disclosure and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds of the disclosure, can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When the compound of the present disclosure is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present disclosure is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutanic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If the compounds of the disclosure simultaneously contain acidic and basic groups in the molecule, the disclosure also includes, in addition to the salt forms mentioned, inner salts or betaines (zwitterions). Salts can be obtained from the compounds of the disclosure by customary methods which are known to the person skilled in the art, for example, by combination with an organic or inorganic acid or base in a solvent or dispersant, or by anion exchange or cation exchange from other salts. The present disclosure also includes all salts of the compounds of the disclosure which, owing to low physiological compatibility, are not directly suitable for use in pharmaceuticals but which can be used, for example, as intermediates for chemical reactions or for the preparation of pharmaceutically acceptable salts.

Furthermore, compounds of the present disclosure may exist in amorphous form and/or one or more crystalline forms, and as such all amorphous and crystalline forms and mixtures thereof of the compounds of the disclosure, including the Examples, are intended to be included within the scope of the present disclosure. In addition, some of the compounds of the instant disclosure may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of this disclosure which results in conversion in vivo to a compound within the scope of this disclosure is also within the scope of this disclosure.

The present disclosure also relates to processes for the preparation of the compounds of the disclosure which are described in the following and by which the compounds of the disclosure are obtainable.

The terms “therapeutically effective (or efficacious) amount” and similar descriptions such as “an amount efficacious for treatment” or “an effective dose” are intended to mean that amount of a compound of the disclosure that will elicit the biological or medical response of a tissue, a system, animal or human that is being sought by a researcher, veterinarian, medical doctor or other clinician. In a preferred embodiment, the term “therapeutically effective amount” means an amount of a compound of the disclosure that alleviates at least one clinical symptom in a human patient. The terms “prophylactically effective (or efficacious) amount” and similar descriptions such as “an amount efficacious for prevention” are intended to mean that amount of a compound of the disclosure that will prevent or reduce the risk of occurrence of the biological or medical event that is sought to be prevented in a tissue, a system, animal or human by a researcher, veterinarian, medical doctor or other clinician.

### Dosages of the Compounds of the Disclosure

The dosage regimen utilizing a compound of the instant disclosure is selected in accordance with a variety of factors including type, species, age, weight, sex and medical condition of the patient; the severity of the condition to be treated; the potency of the compound chosen to be administered; the route of administration, and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compound of the disclosure is within the skill of the art. For administration to a human in the curative or prophylactic treatment of the conditions and disorders identified herein, for example, typical dosages of the compounds of the present disclosure can be about 0.05 mg/kg/day to about 50 mg/kg/day, for example, at least 0.05 mg/kg, at least 0.08 mg/kg, at least 0.1 mg/kg, at least 0.2 mg/kg, at least 0.3 mg/kg, at least 0.4 mg/kg, or at least 0.5 mg/kg, and preferably 50 mg/kg or less, 40 mg/kg or less, 30 mg/kg or less, 20 mg/kg or less, or 10 mg/kg or less, which can be about 2.5 mg/day (0.5 mg/kg×5 kg) to about 50(0) mg/day (50 mg/kg×100 kg), for example. For example, dosages of the compounds can be about 0.1 mg/kg/day to about 50 mg/kg/day, about 0.05 mg/kg/day to about 10 mg/kg/day, about 0.05 mg/kg/day to about 5 mg/kg/day, about 0.05 mg/kg/day to about 3 mg/kg/day, about 0.07 mg/kg/day to about 3 mg/kg/day, about 0.09 mg/kg/day to about 3 mg/kg/day, about 0.05 mg/kg/day to about 0.1 mg/kg/day, about 0.1 mg/kg/day to about 1 mg/kg/day, about 1 mg/kg/day to about 10 mg/kg/day, about 1 mg/kg/day to about 5 mg/kg/day, about 1 mg/kg/day to about 3 mg/kg/day, about 3 mg/day to about 500 mg/day, about 5 mg/day to about 250 mg/day, about 10 mg/day to about 100 mg/day, about 3 mg/day to about 10 mg/day, or about 100 mg/day to about 250 mg/day. Such doses may be administered in a single dose or may be divided into multiple doses.

### Pharmaceutical Compositions

The compounds of the disclosure and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of the said disease or medical condition. As used herein, a subject “in need” of treatment of an existing condition or of prophylactic treatment encompasses both a determination ofneed by a medical professional as well as the desire of a patient for such treatment.

The present disclosure therefore also provides the compounds of the disclosure and their pharmaceutically acceptable salts for use as pharmaceuticals, their use for modulating the activity of mutant KRAS, HRAS and/or NRAS proteins and in particular their use in the therapy and prophylaxis of the below-mentioned diseases or disorders as well as their use for preparing medicaments for these purposes. In certain embodiments, the compounds of the disclosure and their pharmaceutically acceptable salts inhibit the KRAS G12D protein.

Furthermore, the present disclosure provides pharmaceutical compositions which comprise as active component an effective dose of at least one compound of the disclosure and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of the compound of the disclosure and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example, subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of the disclosure and/or its pharmaceutically acceptable salts in the pharmaceutical composition normally is from 0.01 to 200 mg, such as from 0.1 to 200 mg, preferably from 1 to 200 mg, per dose, but depending on the type of the pharmaceutical composition, it can also be higher. In some embodiments, the amount of active compound of the disclosure and/or its pharmaceutically acceptable salts in the pharmaceutical composition is from 0.01 to 10 mg per dose. The pharmaceutical compositions usually comprise 0.5 to 90 percent by weight of the compound of the disclosure and/or their pharmaceutically acceptable salts. The preparation of the pharmaceutical compositions can be carried out in a manner known per se. For this purpose, one or more compounds of the disclosure and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of the disclosure and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of the Disclosure

The present application provides a method of inhibiting RAS-mediated cell signaling comprising contacting a cell with a compound of the disclosure or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in Koff of GTP or a decrease in Koff of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of the disclosure (or their pharmaceutically acceptable salts) or pharmaceutical compositions containing such compounds to treat disease conditions, including but not limited to, conditions implicated by mutant KRAS, HRAS and/or NRAS proteins (e.g., cancer), and in some embodiments the KRAS G12D mutant.

In some embodiments, a method for treatment of cancer is provided, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the cancer is mediated by a KRAS, HRAS or NRAS mutation, e.g., the KRAS G12D mutation. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS, HRAS or NRAS mutation (e.g., KRAS G12D mutation) and if the subject is determined to have the KRAS, HRAS or NRAS mutation, then administering to the subject a therapeutically effective amount of a compound of the disclosure or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound disclosed herein.

KRAS, HRAS or NRAS mutations have also been identified in hematological malignancies (e.g., cancers that affect blood, bone marrow and/or lymph nodes). Accordingly, certain embodiments are directed to administration of the compounds of the disclosure (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkins lymphoma or non-Hodgkins lymphoma. In various embodiments, the compounds are useful for treatment of plasma cell malignancies such as multiple myeloma, mantle cell lymphoma, and Waldenstrom's macroglubunemia.

Determining whbether a tumor or cancer comprises a KRAS, HRAS or NRAS mutation (e.g., the KRAS G12D mutation) can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequences of wild-type human KRAS, HRAS or NRAS are known in the art.

Methods for detecting a mutation in a KRAS, HRAS or NRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays. TaqMan assays, SNP genotyping assays, high resolution melting assays and microarray analyses. In some embodiments, samples are evaluated for KRAS, HRAS or NRAS mutations (e.g., the KRAS G12D mutation) by real-time PCR. In real-time PCR, fluorescent probes specific for the KRAS, HRAS or NRAS mutation are used. When a mutation is present, the probe binds and fluorescence is detected. In some embodiments, the KRAS, HRAS or NRAS mutation is identified using a direct sequencing method of specific regions (e.g., exon 2 and/or exon 3) in the KRAS, HRAS or NRAS gene.

Methods for detecting a mutation in a KRAS, HRAS or NRAS protein (e.g., the KRAS G12D mutation) are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS, HRAS or NRAS mutation (e.g., the KRAS G12D mutation). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of the disclosure, or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (G1ST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, Non-Hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hvpertrophy (BPH)).

In some embodiments, the methods for treatment are directed to treating lung cancers, and the methods comprise administering a therapeutically effective amount of the compounds of the disclosure (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of the disclosure may provide therapeutic benefit for include, but are not limited to, glandular tumors, carcinoid tumors and undifferentiated carcinomas.

The present disclosure also provides methods of modulating a mutant KRAS, HRAS or NRAS protein activity (e.g., activity resulting from the KRAS G12D mutation) by contacting the protein with an effective amount of a compound of the disclosure. Modulation can be inhibiting or activating protein activity. In some embodiments, the present disclosure provides methods of inhibiting protein activity by contacting the mutant KRAS, HRAS or NRAS protein (e.g., KRAS G12D mutant) with an effective amount of a compound of the disclosure in solution. In some embodiments, the present disclosure provides methods of inhibiting the mutant KRAS, HRAS or NRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of the disclosure.

### Combination Therapies

One or more additional pharmacologically active agents may be administered in combination with a compound of the disclosure (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of the disclosure. The additional active agents also include free-acid, free-base and pharmaceutically acceptable salts of said additional active agents. Generally, any suitable additional active agent or agents, including chemotherapeutic agents or therapeutic antibodies, may be used in any combination with the compound of the disclosure in a single dosage formulation (e.g., a fixed dose drug combination), or in one or more separate dosage formulations which allows for concurrent or sequential administration of the active agents (co-administration of the separate active agents) to subjects. In addition, the compounds of the disclosure (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of the disclosure, or a pharmaceutically acceptable salt thereof. In one embodiment, such therapy includes, but is not limited to, the combination of one or more compounds of the disclosure with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAGI agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor a HIF-2a inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate: navelbine: noscapine; teniposide: thaliblastine; vinblastine; vincristine: vindesine; vinflunine: and vinorelbine.

Non-limiting examples of alkylating agents include nitrogen mustards such as chlorambucil, chlomaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07: ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman: procarbazine; temozolomide; treosulfan: and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine: 3-aminopyridine-2-carboxaldehyde thiosemicarbazone; broxuridine; cladribine; cyclophosphamide; cytarabine: emitefur: hydroxyurea; mercaptopurine: nelarabine: pemetrexed; pentostatin: tegafur: and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin: 6-diazo-5-oxo- L-norleucine; aclacinomysins; actinomycin; authramycin; azaserine; bleomycins; cactinomycin; calicheamicin; carabicin; carminomycin; carzinophilin: chromomycins; dactinomycin; detorubicin; esorubicin: esperamicins: geldanamycin; marcellomycin: mitomycins: mitomycin C: mycophenolic acid; olivomycins; novantrone: peplormycin: porfiromycin: potfiromycin: puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4- hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone: abarelix: anastrozole: cetrorelix; deslorelin; exemestane; fadrozole: finasteride: formestane; histrelin (RL 0903); human chorionic gonadotropin: lanreotide; LDI 200 (Milkhaus): letrozole; leuprorelin; mifepristone; nafarelin; nafoxidine: osaterone: prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-nl, interferon alfa-n3, interferon alfacon-1, peginterferon alfa-2a, peginterferon alfa-2b, and leukocyte alpha interferon; interferon beta such as interferon beta-1α, and interferon beta-1b; interferon gamma such as natural interferon gamma-1α, and interferon gamma-1b; aldesleukin; interleukin-1 beta: interleukin-2; oprelvekin: sonermin: tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, allibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER- 2 and Fc MAb (Medarex), ibritumomab tiuxetan, idiotypic 105AD7 MAb (CRC Technology), idiotypic CEa MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L 1 agents or antibodies such as atezolizumab, avelumab, and durvalumab; anti-CTLA-4 agents or antibodies such as ipilumumab; anti-LAG1 agents: and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253: Teramoto, T., et al., 1996, Cancer 77:639-645: Goldstein et al, 1995, Clin. Cancer Res. 1: 1311-1318; Huang, S, M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C₂₂₅ (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478: HIF-2a inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (Uδ 2003/0162712; U.S. Pat. No. 6.413,932); anti-TWEAK agents (U.S. Pat. No. 6.727,225); ADAM distintegrin domain to antagonize the binding of integrin to its ligands (Uδ 2002/0042368); anti-eph receptor and/or anti-ephrin antibodies or antigen binding regions (U.S. Pat. Nos. 5,981,245; 5,728,813; 5,969,110; 6,596,852; 6,232,447; and 6.057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and Rδ 13-0830. Examples of useful matrix metalloproteinase inhibitors are described, for example, in WO 96/33172, WO 96/27583, EP 1004578, WO 98/07697. WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, Uδ 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP- 7, MMP- 8, MMP-10, MMP-11, MMP-12, and MMP-13).

Non-limiting examples of VEGF and VEGFR inhibitory agents include bevacizumab, cediranib. CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAPT™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib. AZD 9935, BAY REδ 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University. US), E 7820, EHT 0101, endostatin, enzastaurin hydrochloride, ER-68203-QQ(IVAX, US), fibrinogen-E fragment, Flk-1 (ImClone Systems, US), forms of FLT 1 (VEGFR 1), FR-II1142, GCS-100, GW 2286 (GlaxoSmithKline. UK), IL-8, ilomastat. IM-862, irsogladine. KM-2550 (Kyowa Hakko, Japan), lenalidomide, lenvatinib, MAb alpha5beta3 integrin, second generation (Applied Molecular Evolution, USA and Medlmmune, US), MAb VEGF (Xenova. UK), marimastat, maspin (Sosei, Japan), metastatin, motuporamine C, M-PGA, ombrabulin, OXI4503, PI 88, platelet factor 4. PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L 1 antagonist. In embodiments, the additional anti-cancer agent(s) is a RAF inhibitor, EGFR inhibitor, MEK inhibitor, ERK inhibitor, PI3K inhibitor, AKT inhibitor. TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAGl, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LT1462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424: buparlisib; CAL263; copanlisib; CUDC-907; dactolisib (WO 06/122806); demethoxyiridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082: WO 09/055,730); PIK 90; PWT33597; SF1126; sonolisib: TGI 00-115: TGX-221: XL 147: XL-765; wortmannin: and ZSTK474.

Non-limiting examples of AKT inhibitors include Akt-1-1 (inhibits Aktl) (Barnett et al. (2005) Biochem. J, 385 (Pt. 2), 399408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-1H-imidazo[4,5-c]pyridiny1 compounds (e.g., WO5011700); indole-3-carbinol and derivatives thereof (e.g., U.S. Pat. No. 6,656,963; Sarkar and Li (2004) J Nuir. 134(12 Suppl), 3493S-3498S); perifosine, Dasmahapatra et al. (2004) Clin. Cancer Res, 10(15), 5242-52, 2004); phosphatidylnositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1.2-c]pyrido[3.4-e][1.3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1: TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-13-hydroxy(hydroxymethyl)methylpropanoatel-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2.2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflomithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride. HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyl)inic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin 1 (Harvard University, US), urethan, valspodar, verteporfin, zoledronic acid, and zosuquidar.

The present disclosure further provides a method for using the compounds of the disclosure or pharmaceutical compositions provided herein, in combination with radiation therapy to treat cancer. Techniques for administering radiation therapy are known in the art, and these techniques can be used in the combination therapy described herein. The administration of the compound of the disclosure in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of the disclosure, or a pharmaceutically acceptable salt thereof. In one embodiment, such therapy includes, but is not limited to, the combination of one or more compounds of the disclosure with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of the disclosure and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the disclosure can be administeredjust followed by and any of the agents described above, or vice versa. In some embodiments of the separate administration protocol, a compound of the disclosure and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of the disclosure, and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of the disclosure, or the pharmaceutically acceptable salt thereof, for use in therapy, or use of the compound of the disclosure, or the pharmaceutically acceptable salt thereof, in therapy. The present disclosure also provides for the compound of the disclosure, or the pharmaceutically acceptable salt thereof, for use in treating cancer, or use of a compound of the disclosure, or the pharmaceutically acceptable salt thereof, for treating cancer. The present disclosure also provides for the compound of the disclosure, or the pharmaceutically acceptable salt thereof, for the preparation of a medicament for the treatment of cancer, or use of the compound of the disclosure, or the pharmaceutically acceptable salt thereof, for the preparation of a medicament for the treatment of cancer. The present disclosure also provides for the compound of the disclosure, or the pharmaceutically acceptable salt thereof, and an additional anti-cancer agent, for use in the treatment of cancer, or use of the compound of the disclosure, or the pharmaceutically acceptable salt thereof, and the additional anti-cancer agent for treating cancer. The disclosure also provides the compound of the disclosure, or the pharmaceutically acceptable salt thereof, and an additional anti-cancer agent, for the preparation of a medicament for the treatment of cancer, or use of the compound of the disclosure, or the pharmaceutically acceptable salt thereof, and the additional anti-cancer agent, for the preparation of a medicament for the treatment of cancer. The present disclosure also provides for a pharmaceutical composition comprising the compound of the disclosure, or the pharmaceutically acceptable salt thereof, for use in the treatment of cancer, or use of the pharmaceutical composition comprising the compound of the disclosure, or the pharmaceutically acceptable salt thereof, for treating cancer. The present disclosure also provides for a pharmaceutical composition comprising the compound of the disclosure, or the pharmaceutically acceptable salt thereof, and an additional anti-cancer agent, for use in the treatment of cancer, or use of the pharmaceutical composition comprising the compound of the disclosure, or the pharmaceutically acceptable salt thereof, and the additional anti-cancer agent, for treating cancer.

### Methods of Prepanng the Compounds of the Disclosure

The compounds described herein can be prepared according to the procedures of the following schemes and examples, using appropriate materials and are further exemplified by the following specific examples. The compounds illustrated in the examples are not, however, to be construed as forming the only genus that is considered as the disclosure. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Any intermediates described below may be referred to herein by their number preceded by “Int-.”

Throughout the synthetic schemes and examples, abbreviations and acronyms may be used with the following meanings unless otherwise indicated: AcOH=acetic acid; anhydr. =Anhydrous; aq. =aqueous, atm=atmosphere; BM5=borane dimethylsulfide: BnBr=(bromomethyl)benzene: Bodipy-GDP=mixture of ((2R,3S,4R,5R)-5-(2-amino-6-oxo-1,6-dihydro-9H-purin-9-yl)-3-(((2-(3-(5,5-difluoro-7,9-dimethyl-5H-414,514-dipyrrolo[1.2-c:2′,1′-f][1.3,2]diazaborinin-3-yl)propanamido)ethyl)carbamoyl)oxy)-4-hydroxytetrahydrofuran-2-yl)methyl hydrogen diphosphate and ((2R,3R,4R,5R)-5-(2-amino-6-oxo-1,6-dihydro-9H-purin-9-yl)-4-(((2-(3-(5,5-difluoro-7,9-dimethyl-5H-414,514-dipyrrolo[1.2-c:2′,1′-f][1.3,2]diazaborinin-3-yl)propanamido)ethyl)carbamoyl)oxy)-3-hydroxytetrahydrofuran-2-yl)methyl hydrogen diphosphate (Invitrogen™, catalog number G22360): Boc₂O=Di-tert-butyl dicarbonate: BOP=benzotriazol-1-yloxytris(dimethylamino)phosphonium hexafluorophosphate: B₂pin₂=Bis(pinacolato)diboron; br s=broad singlet; Bu=butyl; t-Bu=tert-butyl; cataCXium A Pd G3=Mesylate[(di(1-adamantyl)-n-butylphosphine)-2-(2′-amino-1,1′-biphenyl)]palladium(II); cataCxium® C=trans-Di(p-acetato)bis|o-(di-o-tolylphosphino)benzyldipalladium(II): CDCl₃=deuterated chloroform; Cbz=carbobenzyl; CDI=1.1′-carbonyldiimidazole; CELITE=diatomaceous earth; CF₃=trifluoromethyl; cGMP=cyclic guanosine monophosphate; CH₃NO₂=nitromethane; COD=1.5-cyclooctadiene: conc. =concentrated: CPME=cyclopentyl methyl ether; CPhos Pd G3=[(2-Dicyclohexylphosphino-2′,6′-bis(N,N-dimethylamino) -1,1′-biphenyl)-2-(2′-amino-1,1′-biphenyl)] palladium(II) methanesulfonate; Cphos Pd G4=2-(2-dicyclohexylphosphanylphenyl)-1-N,1-N,3-N,3-N-tetramethylbenzene-1,3-diamine:methanesulfonic acid; N-methyl-2-phenylaniline; palladium; Cy₂NMe=N-cyclohexyl-N-methyl)cyclohexanamine; DAST=(Diethylamino)sulfur trifluoride; DBU=1.8-diazabicyclo[5.4.0]undec-7-ene: DCM=dichloromethane; DCE=dichloroethane; DHP=3.4-dihydropyran; DIA=diisopropylamine; DIAD=Diisopropyl azodicarboxylate; DIEA/DIPEA/NEt₃=N,N-Diisopropylethylamine=N-ethyl-N-isopropylpropan-2-amine; DMAP =dimethylaminopyridine=N,N-dimethylpyridin-4-amine; DME=dimethoxyethane; DMEA=N,N-Dimethylethanamine; DMF=N,N-dimethylformamide; DM1=1,3-Dimethyl-2-imidazolidinone; DMP=Dess-Martin periodinane; DMS=dimethylsulfide; DMSO=dimethylsulfoxide; DPPF or dppf=1,1′-bis(diphenylphosphino)ferrocene: EDCI=1-ethyl-3-(3-dimethylaminopropyl)carbodiimide; EDTA=ethylenediaminetetraacetic acid: equiv, eq. =equivalent(s); Et=ethyl; Et₃N=triethylamine; EtOAc=ethyl acetate; EtOH=ethanol; Grubbs Catalyst=(1,3-Bis(2,4,6-trimethylphenyl)-2-imidazolidinyldene)dichloro(phenylmethylene)(tricyclohexylphosphine)ruthenium; GTP=guanosine triphosphate; h=hour; HATU=1-[bis(dimethylamino)methylene]-1H-1,2,3-triazolo[4,5-b]pyridinium 3-oxid hexafluorophosphate: HBpin=4,4,5,5-tetramethyl-1,3,2-dioxaborolane; HEPES=4-(2-hydroxyethyl)-1-piperazineethanesulfonic acid; HMDS=hexamethydisilazane; HMPT=hexamethylphosphoramide; HPLC=High pressure liquid chromatography; Int. =intermediate; iPr=isopropyl; IP=inflection points; i-PrOH=IPA=Isopropanol; iPrMgCl LiCl=isopropylmagnesium chloride lithium chloride; KHMDS=Potassium bis(trimethylsilyl)amide: LAH=lithium aluminum hydride: LCMS, LC/MS=liquid chromatography-mass spectrometry: min, min. =minute; LDA=lithium diisopropylamide; LiHMDS=lithium bis(trimethylsilyl)amide. M=Molar; m-CPBA or mCPBA=3-chlorobenzoperoxoic acid: Me=methyl: MeCN, ACN=acetonitrile; MeOH=methanol; mp, m.p. =melting point; mpk=milligrams per kilogram: MsC11=Methanesulfonyl chloride; MO=methoxy; MOM=methoxymethyl; MOMCl=chloromethyl methyl ether; MAS=mass spectrometry: MTBE=methyl tert-butyl ether; N=Normal; NaOMe=sodium methoxide; NaHMDS=sodium hexamethyldisilazide: NBS=N-bromosuccinimide=1-bromopyrrolidine-2,5-dione; nBuLi=n-Butyl)ithium; NCS=N-chlorosuccinimide; N1S=N-iodosuccinimide: NMO=4-Methylmorpholine N-oxide; NMP=N-methylpyrrolidone; NMR=nuclear magnetic resonance; N.D. =not determined; OAc=acetate; PDA=photodiode array; PDC=pyridinium dichromate; Pet, ether =petroleum ether; Pd—C=palladium on carbon; Pdz(dba)₃=tris(dibenzyldeneacetone)dipalladium (0); Ph=phenyl; phen=1.10-phenanthroline; PMB=para-methoxybenzyl=4-methoxybenzyl: Pr=propyl: psi =pounds per square inch gauge: POCI₃=phosphorus(V) oxide chloride: PPTS=pyridinium p-toluenesulfonate; PTLC, prep TLC=preparative thin layer chromatography; PTSA=p-tolunesulfonic acid; pTsOH=p-toluenesulfonic acid; rac=racemic; rac-BINAP=(R)-(+)-2,2′-bis(diphenylphosphino)-1,1′-binaphthyl; RT=retention time; RP-HPLC=reverse phase HPLC; rt=room temperature; RuPhos Pd G2=2nd Generation RuPhos Precatalyst; Chloro(2-dicyclohexylphosphino-2′0,6′-diisopropoxy-1,1′-biphenyl)[2-(2′-amino-1,1′-biphenyl)]palladium(II): sat. =saturated; SelecFluor=l-chloromethyl-4-fluoro-1,4-diazoniabicyclo[2.2.2]octane bis(tetrafluoroborate): SFC=supercritical fluid chromatography: SEM=(2-(trimethylsilyl)ethoxy)methyl; SOS=Son of Sevenless: SPhos Pd G3=(2-Dicyclohexylphosphino-2′,6′-dimethoxybiphenyl) [2-(2′-amino-1,1′-biphenyl)]palladium(II) methanesulfonate; TBAF=tetra-n-butylammonium fluoride; TBS=tert-butyldimethylsilane: TBSC1=tert-butyldimethylsilyl chloride: TBDPS-CI=tert-Butyl(chloro)diphenylsilane; t-BuOK=potassium tert-butoxide; t-BuONa=sodium tert-butoxide; TEA=triethylamine: TEMPO=2,2,6,6-Tetramethylpiperidine 1-oxyl; Tf=trifluoromethanesulfonate; Tf20=triflic anhydride; TFA=trifluoroacetic acid: TFAA=trifluoroacetic anhydride: THF=tetrahydrofuran: THP=tetrahydropyran; TIPS=triisopropylsilane; TIPSCCBr=(bromoethynyl)triisopropylsilane: TLC=thin layer chromatography: TMEDA=N,N,N′,N′-Tetramethylethylenediamine; TMP=2,2,6,6-tetramethylpiperidine; TMS=trimethylsilyl; TMSCH. =trimethyl(trifluoromethyl)silane; TPAP=tetrapropylammonium perruthenate: TsOH=tosylc acid: TWEEN=polyoxyethylene (20) sorbitan monolaurate; VCD=vibrational circular dichroism; v, v/v=volume, volume to volume; w, w/w=weight, weight to weight, XPhos=2-dicyclohexylphosphino-2′,4′,6′-triisopropylbiphenyl; XPhos Pd G3=(2-Dicyclohexylphosphino-2′,4′,6′-triisopropyl-1,1′-biphenyl)[2-(2′-amino-1,1′-biphenyl)]palladium(II) methanesulfonate; μm=micrometer.

### General Schemes

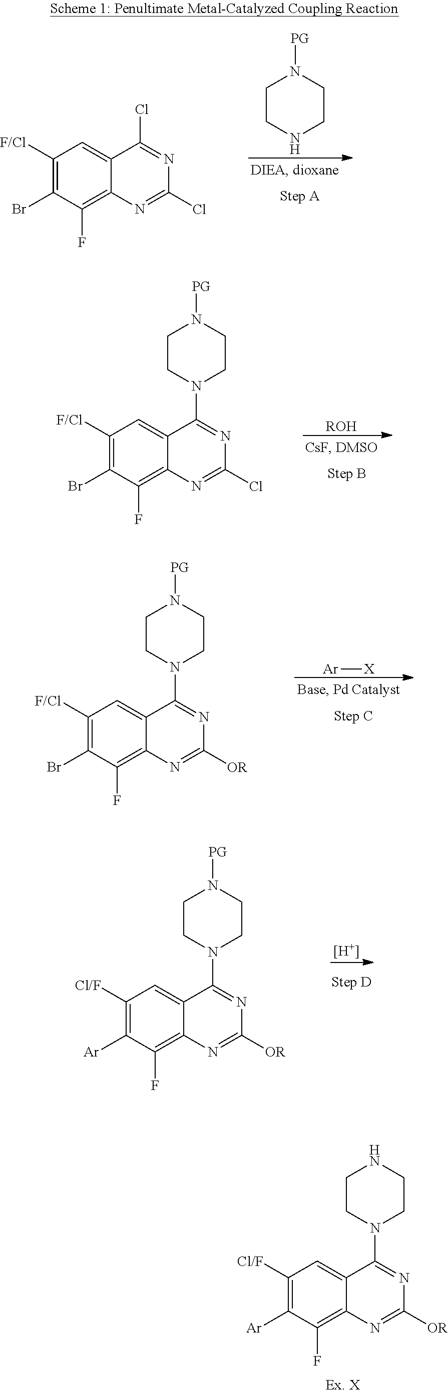

Scheme 1 illustrates one procedure for preparing the compounds of the disclosure using a metal-catalyzed reaction to prepare the appropriately substituted quinazoline. Displacement of the 4-chloro group of a dichloro-substituted quinazoline with a protected piperazine (wherein PG is a suitable amine-protecting group) provides a monochloro-substituted quinazoline intermediate. Displacement of the second chloro group of the quinazoline with an appropriate alcohol provides an ether-substituted quinazoline intermediate. This intermediate is then coupled with a suitable aryl/heteroaryl halide using a Pd-based catalyst. Removal of the protecting group on the piperazine moiety, using, for example, acidic conditions yields the compound of the disclosure.

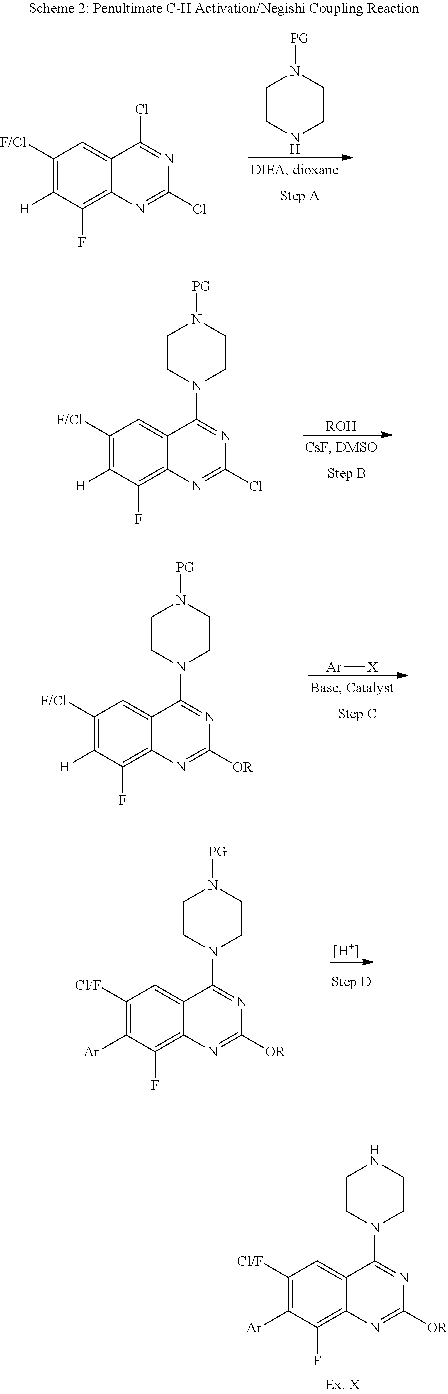

Scheme 2 illustrates another procedure for preparing the compounds of the disclosure. In this procedure, a C—H Activation and Negishi coupling reaction is used to provide aryl substitution on the quinazoline ring. The remaining steps are similar to those described above for the process of Scheme 1.

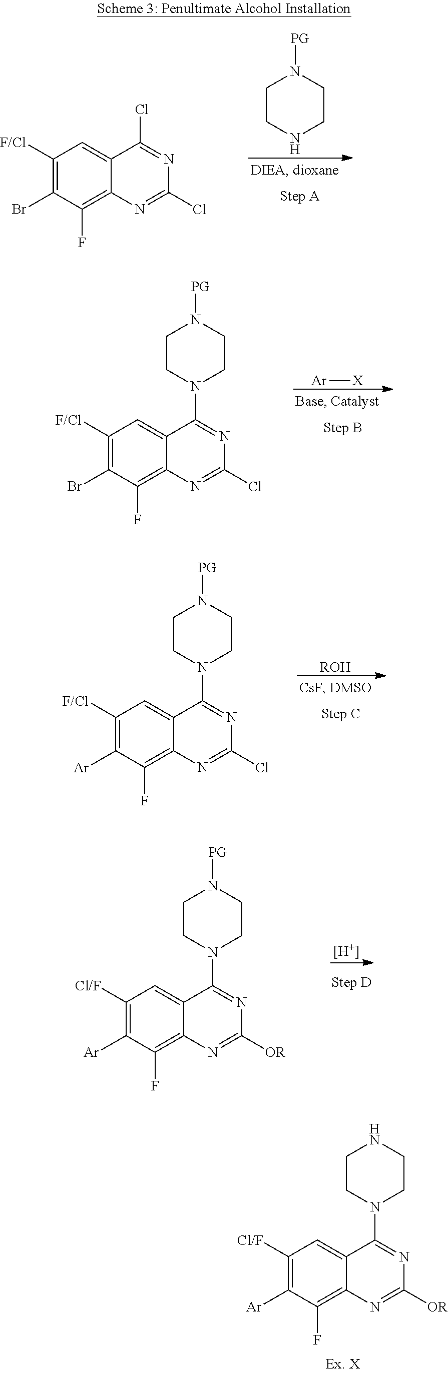

Scheme 3 illustrates another procedure for preparing the compounds of the disclosure. In this procedure, a Negishi coupling reaction occurs before displacement of the 2-chloro group of the quinazoline. The remaining steps are similar to those described above for the processes of Schemes 1 and 2.

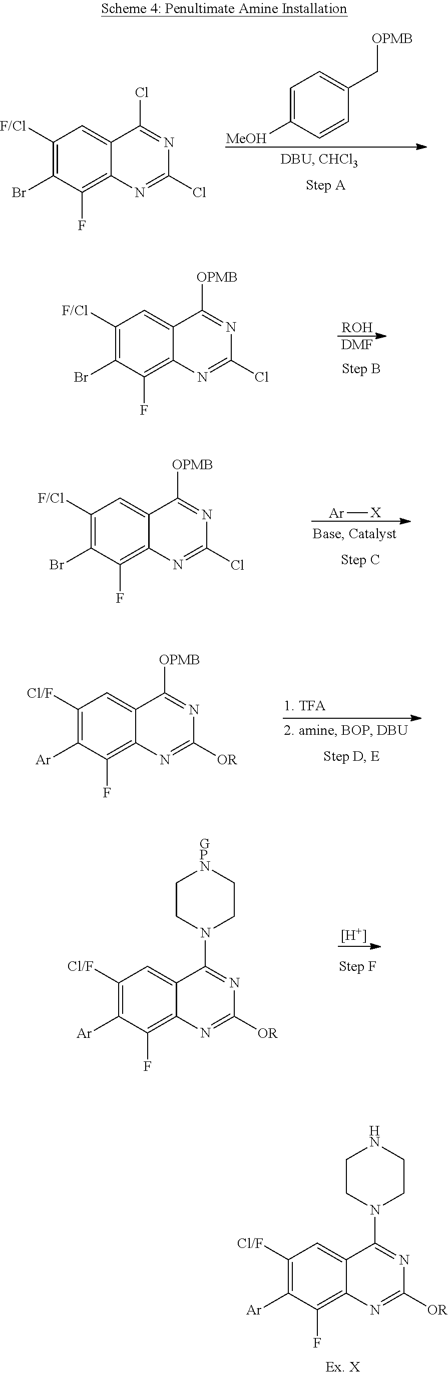

Scheme 4 illustrates another procedure for preparing the compounds of the disclosure. In this procedure, a para methoxy benzyl (PMB) protecting group displaces the 4-chloro group of adichloro-substituted quinazoline. Installation of the alcohol and aryl groups proceed similarly as above. Removal of the PMB group is then followed by piperazine installation using BOP and DBU and a final deprotection step.

### Examples

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]⁺ ion unless otherwise noted. ¹H NMR spectra were recorded at 400-600 MHz at ambient temperature unless otherwise noted. Protons reported as 0.5 H are due to rotameric signals. RP-HPLC refers to reverse-phase HPLC on C18-functionalized preparative or semi-preparative columns with gradient elution using acetonitrile and water modified with trifluoroacetic acid or ammonium hydroxide as eluents and fractions were lyophilized or concentrated by rotary evaporation unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables. Certain products/intermediates in the examples include indication of “Peak 1” and/or “Peak 2”, which refer to the order of elution of the indicated product/intermediate from the chromatography column (e.g., an SFC column) used to isolate the compound under the specified conditions. Thus, for example, Peak 1 refers to the first eluting compound, e.g., first eluting stereoisomer, under the specified conditions.

SFC Columns used in the chiral resolution of stereoisomers are summarized in the following Table:

| | SFC Column |
|---|---|
| SFC Column | Abbreviation |
| Daicel Chiralpak AD (30 mm × 250 mm, 10 um) | Column A |
| AD-H (21 mm × 250 mm, 5 um) | Column B |
| Daicel Chiralpak IC (30 mm × 250 mm, 10 um) | Column C |
| Phenomenex, Cellulose-2 (30 mm × 250 mm, 5 um) | Column D |
| Regis, (R,R) Whelk-O1 (21 mm × 250 mm, 5 um) | Column E |
| Phenomenex, LUX-4 (21 mm × 250 mm, 5 um) | Column F |
| ES Industries, CCAF4 (21 mm × 250 mm, 5 um) | Column G |
| CCC (21 mm × 250 mm, 5 um) | Column H |
| Phenomenex, LUX-2 (21 mm × 250 mm, 5 um) | Column I |
| Daicel Chiralpak AS (50 mm × 250 mm, 10 um) | Column J |
| Daicel Chiralpak IG (30 mm × 250 mm, 10 um) | Column K |
| Chiral Technologies, IB-N (21 mm × 250 mm, 5 um) | Column L |
| Daicel Chiralpak IA (21 mm × 250 mm, 5 um) | Column M |
| Daicel Chiralpak OJ-H (30 mm × 250 mm, 5 um) | Column N |
| Phenomenex, Cellulose-2 (25 mm × 250 mm, 10 um) | Column O |
| Daicel Chiralpak IG-3 (4.6 mm × 100 mm, 3 um) | Column P |
| CCA (21 mm × 250 mm, 5 um) | Column Q |
| CCO (21 mm × 250 mm, 5 um) | Column R |

### Intermediate Syntheses

### Intermediate A4: tert-butyl 3-(6,8-difluoro-2-((1-(morpholinomethyl)cyclopropyl)methoxy)quinazolin-4-yl)-3,8-diazabicyclo[3.2.1]octane-8-carboxylate (Int-A4)

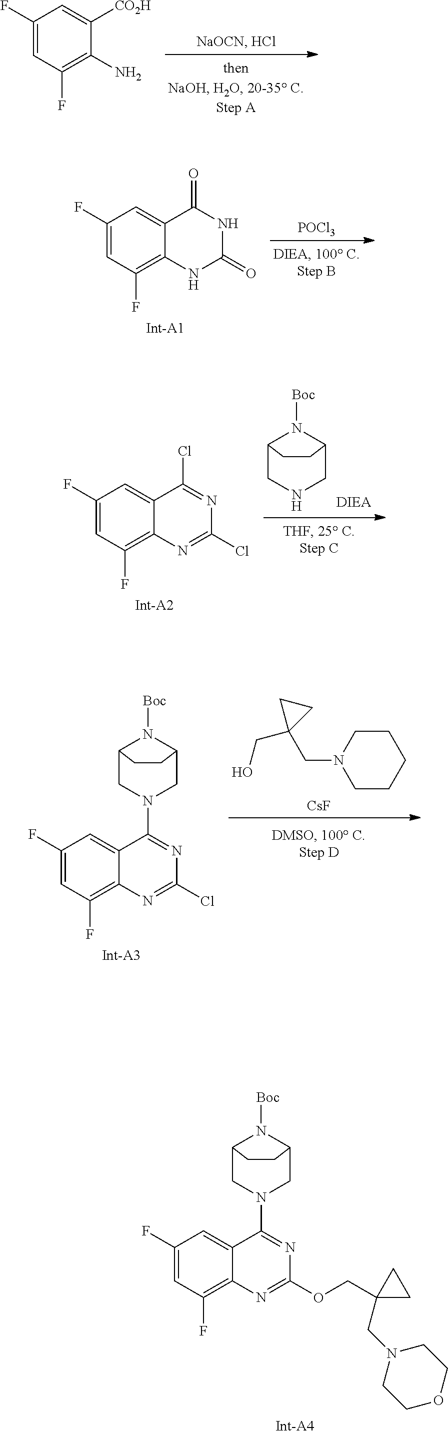

### Step A: 6.8-difluoroquinazoline-2,4(1H,3H)-dione (Int-A1)

Sodium hydroxide (67.9 g, 1.70 mol) was dissolved in H₂O (2.10 L) in a 5 L three-necked round bottom flask at 20° C. To this mixture was added 2-amino-3,5-difluorobenzoic acid (210 g, 1.21 mol) at 20° C. The resulting mixture was stirred at 35° C. for 1 h. To this mixture was added sodium isocyanate (173 g, 2.67 mol) at 35° C. and the mixture was stirred until the solid dissolved completely. The resulting mixture was stirred at 35° C. for 1 h. To this mixture was added HCl (6 M, 1.01 L, 6.06 mol) dropwise at 30-35° C. for 1 h and the pH was maintained between 6 and 7. Then NaOH (146 g, 3.64 mol) was added at 25° C. for 1 h. The resulting mixture was stirred at 25° C. for 9 h. To this mixture was added HCl (12 N) dropwise at 20-25° C. and the pH was maintained at -1 to 2. Acetone (1.2 L) was added into the mixture and the mixture was stirred at 25° C. for 1 h. The precipitate was removed by filtration and the filter cake was washed with acetone (500 mL). To the filtrate was added methanol (1.2 L) and the mixture was stirred at 25° C. for 1 h. The product was obtained by collection of the precipitate to afford 6.8-difluoroquinazoline-2,4(1H,3H)-dione (Int-A1). ¹H NMR (400 MHz DMSO-d₆) δ 11.54 (s, 1H), 11.34 (s, 1H), 7.71-7.72 (m, 1H), 7.46-7.49 (m, 1H).

### Step B: 2,4-dichloro-6,8-difluoroquinazoline (Int-AZ)

A 5 L three-necked round bottom flask was charged with 6.8-difluoroquinazoline-2,4(1H,3H)-dione (Int-A1) (166 g, 838 mmol) and POCl₃ (1.03 kg, 6.70 mol) at 20° C. The resulting mixture was cooled to 10° C. and DIEA (324 g, 2.51 mol) was added. The reaction mixture was stirred at 100° C. for 3 h. The reaction mixture was evaporated in vacuo to dryness. The residue was dissolved in EtOAc (1 L) and washed with sat. NaHCO₃ (1 L). The organic layer was washed with brine (300 mL×2), dried with Na₂SO₄ and filtered. The filtrate was concentrated in vacuo to dryness. The residue was triturated with MTBE (300 mL) at 25° C. for 30 min. The mixture was filtered and the solid was collected to afford 2,4-dichloro-6,8-difluoroquinazoline (Int-A2). ¹H NMR (400 MHz DMSO-d₆) δ 8.24-8.29 (m, 1H), 7.97-7.99 (m, 1H).

### Step C: tert-butyl 3-(2-chloro-6,8-difluoroquinazolin-4-yl)-3,8-diazabicyclo[3.2.1]octane-8-carboxylate (Int-A3)

A 1 L three-necked round bottom flask was charged with DIEA (39.6 g, 306 mmol) in THF (720 mL) at 25° C. 2.4-Dichloro-6,8-difluoroquinazoline (Int-A2) (24.0 g, 102 mmol) was added at 25° C. The resulting mixture was stirred at 25° C. for 5 min. tert-Butyl 3.8-diazabicyclo[3.2.1]octane-8-carboxylate (22.7 g, 107 mmol) was added into the reaction mixture at 25° C. and the reaction mixture was stirred for 1 h. Water (100 mL) was added. The resulting mixture was extracted with ethyl acetate (100 mL×2). The organic phase was washed with brine (150 mL) and dried with anhydrous Na₂SO₄. The organic layer was filtered and concentrated in vacuo. The residue was triturated with petroleum ether/MTBE (v/v=2/1, 130 mL) at 25° C. for 30 min. The resulting mixture was filtered and the solid was collected to afford tert-butyl 3-(2-chloro-6,8-difluoroquinazolin-4-yl)-3,8-diazabicyclo[3.2.1]octane-8-carboxylate (Int-A3).

¹H NMR (400 MHz DMSO-d₆) δ: 7.85-7.90 (m, 1H), 7.69-7.71 (m, 1H), 4.33-4.36 (m, 2H), 4.24 (s, 2H), 3.57-3.59 (d, 2H, J=8.0 Hz), 1.78-1.79 (m, 2H), 1.63-1.65 (m, 2H), 1.41-1.46 (m, 9H).

### Step D: tert-butyl 346,8-difluoro-2-((1-(morpholinomethyl)cyclopropyl)methoxy)quinazolin-4-yl)-3,8-diazabicyclo[3.2.1]octane-8-carboxylate (Int-A4)

To a vial with tert-butyl 3-(2-chloro-6,8-difluoroquinazolin-4-yl)-3,8-diazabicyclo[3.2.1]octane-8-carboxylate (Int-A3) (1.0 g, 2.43 mmol) were added dry CsF (1.48 g, 9.74 mmol), (1-(morpholinomethyl)cyclopropyl)methanol (0.834 g, 4.87 mmol) and DMSO (16.0 mL). The mixture was heated at 100° C. for 6 hours. The reaction mixture was cooled to room temperature and diluted with water (10 mL) and extracted with EtOAc (50 mL×2). The organic layers were combined and dried with MgSO₄ and evaporated in vacuo to dryness. The crude material was purified via silica gel column chromatography (0-100% EtOAc in Hexanes) to yield tert-butyl 3-(6,8-difluoro-2-((1-(morpholinomethyl)cyclopropyl)methoxy)quinazolin-4-yl)-3,8-diazabicyclo[3.2.1]octane-8-carboxylate (Int-A4). MS (ESI): m/z (M+H)+546.

### Intermediate D3 tert-butyl (1S,5R)-3-(2-chloro-6,8-difluoroquinazolin-4-yl)-1-methyl-3,8-diazabicyclo[3.2.1]octane-8-carboxylate (Int-D3)

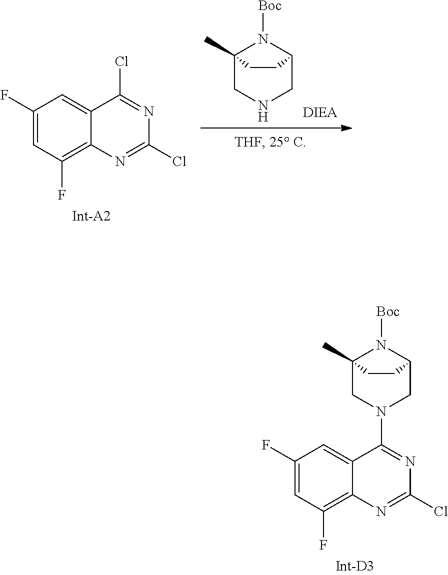 [SMILES: Fc1cc(F)c2nc(Cl)nc(Cl)c2c1 ; CC(C)(C)OC(=O)N1[C@@H]2CC[C@@]1(C)CNC2 ; CC(C)(C)OC(=O)N1[C@@H]2CC[C@@]1(C)CN(c1nc(Cl)nc3c(F)cc(F)cc13)C2]

Int-D3 was prepared analogously to Int-A3 above.

The compounds in the table below were synthesized via a similar procedure as used for preparing Int-A4.

| | | | [M + H]⁺ |
|---|---|---|---|
| Int | Structure | Compound Name | Found |
| Int-B4 | 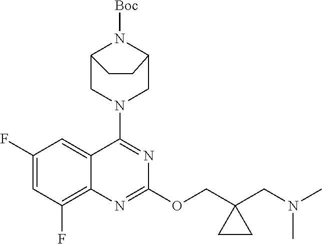 [SMILES: CN(C)CC1(COc2nc(N3CC4CCC(C3)N4C(=O)OC(C)(C)C)c3cc(F)cc(F)c3n2)CC1] | tert-butyl 3-(2-((1- ((dimethylamino)methyl)cyclopropyl)methoxy)- 6,8-difluoroquinazolin-4-yl)-3,8- diazabicyclo[3.2.1]octane-8-carboxylate | 504 |
| Int-C4 | 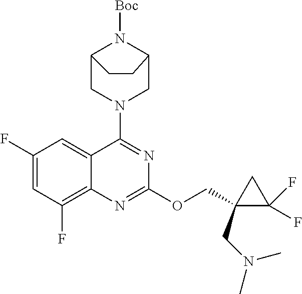 [SMILES: CN(C)C[C@@]1(COc2nc(N3CC4CCC(C3)N4C(=O)OC(C)(C)C)c3cc(F)cc(F)c3n2)CC1(F)F] | tert-butyl 3-(2-(((R)-1-((dimethylamino)methyl)- 2,2-difluorocyclopropyl)methoxy)-6,8- difluoroquinazolin-4-yl)-3,8- diazabicyclo[3.2.1]octane-8-carboxylate | 540 |
| Int-D4 | 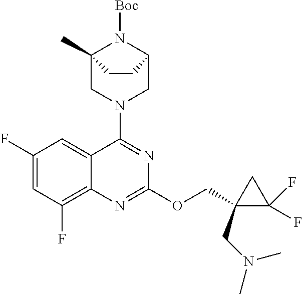 [SMILES: CN(C)C[C@@]1(COc2nc(N3C[C@H]4CC[C@@](C)(C3)N4C(=O)OC(C)(C)C)c3cc(F)cc(F)c3n2)CC1(F)F] | tert-butyl (1S,5R)-3-(2-(((R)-1- ((dimethylamino)methyl)-2,2- difluorocyclopropyl)methoxy)-6,8- difluoroquinazolin-4-yl)-1-methyl-3,8- diazabicyclo[3.2.1]octane-8-carboxylate | 554 |
| Int-E4 | 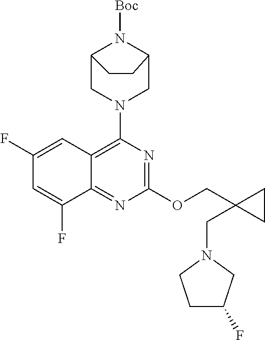 [SMILES: CC(C)(C)OC(=O)N1C2CCC1CN(c1nc(OCC3(CN4CC[C@@H](F)C4)CC3)nc3c(F)cc(F)cc13)C2] | tert-butyl 3-(6,8-difluoro-2-((1-(((R)-3- fluoropyrrolidin-1- yl)methyl)cyclopropyl)methoxy)quinazolin-4-yl)- 3,8-diazabicyclo[3.2.1]octane-8-carboxylate | 548 |
| Int-F4 | 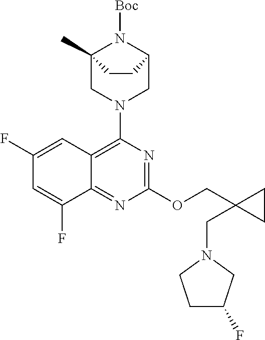 [SMILES: CC(C)(C)OC(=O)N1[C@@H]2CC[C@@]1(C)CN(c1nc(OCC3(CN4CC[C@@H](F)C4)CC3)nc3c(F)cc(F)cc13)C2] | tert-butyl (1S,5R)-3-(6,8-difluoro-2-((1-(((R)-3- fluoropyrrolidin-1- yl)methyl)cyclopropyl)methoxy)quinazolin-4-yl)- 1-methyl-3,8-diazabicyclo[3.2.1]octane-8- carboxylate | 562 |
| Int-G4 | 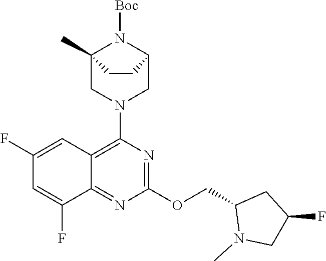 [SMILES: CN1C[C@H](F)C[C@H]1COc1nc(N2C[C@H]3CC[C@@](C)(C2)N3C(=O)OC(C)(C)C)c2cc(F)cc(F)c2n1] | tert-butyl (1S,5R)-3-(6,8-difluoro-2-(((2S,4R)-4- fluoro-1-methylpyrrolidin-2- yl)methoxy)quinazolin-4-yl)-1-methyl-3,8- diazabicyclo[3.2.1]octane-8-carboxylate | 522 |
| Int-H4 | 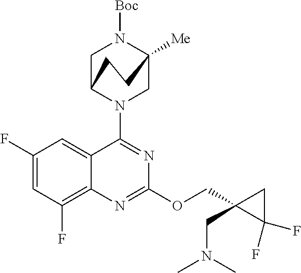 [SMILES: CN(C)C[C@@]1(COc2nc(N3C[C@]4(C)CC[C@H]3CN4C(=O)OC(C)(C)C)c3cc(F)cc(F)c3n2)CC1(F)F] | tert-butyl (1S,4S)-5-(2-(((R)-1- ((dimethylamino)methyl)-2,2- difluorocyclopropyl)methoxy)-6,8- difluoroquinazolin-4-yl)-1-methyl-2,5- diazabicyclo[2.2.2]octane-2-carboxylate | 554 |
| Int-I4 | 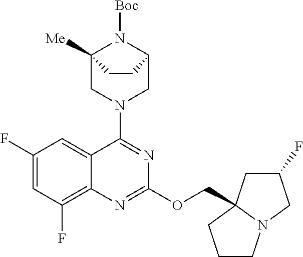 [SMILES: CC(C)(C)OC(=O)N1[C@@H]2CC[C@@]1(C)CN(c1nc(OC[C@]34CCCN3C[C@@H](F)C4)nc3c(F)cc(F)cc13)C2] | tert-butyl (1S,5R)-3-(6,8-difluoro-2-(((2S,7aR)-2- fluorotetrahydro-1H-pyrrolizin-7a(5H)- yl)methoxy)quinazolin-4-yl)-1-methyl-3,8- diazabicyclo[3.2.1]octane-8-carboxylate | 548 |

### Intermediate K3: tert-butyl 3-(2,6-dichloro-8-fluoroquinazolin-4-yl)-3,8-diazabicyclo[3.2.1]octane-8-carboxylate (Int-K3)

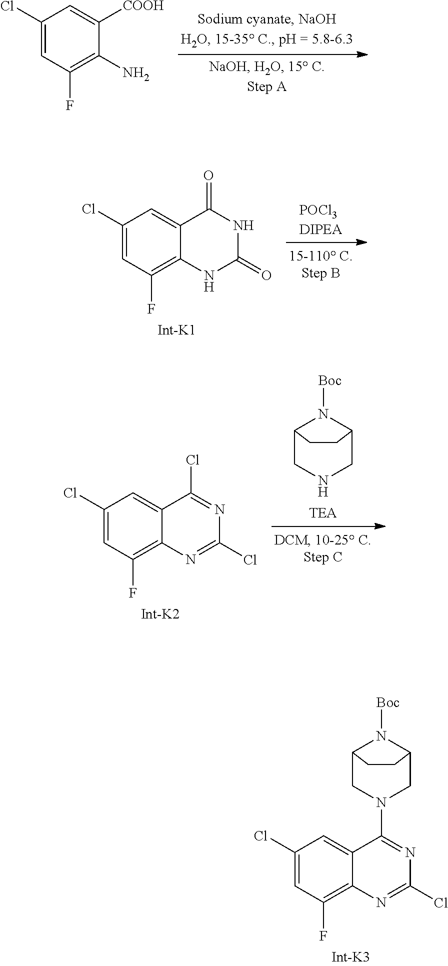 [SMILES: Nc1c(F)cc(Cl)cc1C(=O)O ; O=c1[nH]c(=O)c2cc(Cl)cc(F)c2[nH]1 ; "POCl3DIPEA" ; Fc1cc(Cl)cc2c(Cl)nc(Cl)nc12 ; CC(C)(C)OC(=O)N1C2CCC1CNC2 ; CC(C)(C)OC(=O)N1C2CCC1CN(c1nc(Cl)nc3c(F)cc(Cl)cc13)C2]

### Step A: 6-chloro-8-fluoroquinazoline-2,4(1H,3H)-dione (Int-K1)

2-Amino-5-chloro-3-fluorobenzoic acid (165 g, 870 mmol) and sodium cyanate (124 g, 1.91 mol) were added into a solution of NaOH (49.0 g, 1.22 mol) in water (1.65 L) at 15-20° C. The reaction mixture was stirred at 30-35° C. for 1 h. To this mixture was added NaOH (49.0 g, 1.22 mol) at 30-35° C. The mixture was stirred at 30-35° C. for 4 h (pH=5.8-6,3). To this mixture was added a solution of NaOH (115 g, 2.87 mol) in water (1.65 L). The reaction mixture was stirred at 20-30° C. for 12 h. The pH of the product mixture was adjusted with HCl (12 N) to 1-2 at 10-20° C. Acetone (3 L) was added into the mixture. The precipitate was collected by filtration and the solid obtained was washed with MeOH (3 L) and dried at 45° C. for 24 h to afford 6-chloro-8-fluoroquinazoline-2,4(1H,3H)-dione (Int-K1). ¹H NMR (400 MHz, DMSO-d₆) δ 11.56 (s, 1H), 11.43 (s, 1H), 7.80 (d, J=2.4 Hz, 1H), 7.65 (s, 1H).

### Step B: 2,4,6-trichloro-8-fluoroquinazoline (Int-K2)

6-Chloro-8-fluoroquinazoline-2,4(1H,3H)-dione (Int-K1) (100 g, 466 mmol) was added into POCl₃ (643 g, 4.19 mol) at 15-25° C. The reaction mixture was stirred 15-25° C. for 2 h. DIPEA (30.1 g, 233 mmol) was added into the mixture at 15-25° C. under N2. The resulting mixture was heated to 100-110° C. and stirred for 30 h. The reaction mixture was concentrated in vacuo to dryness. The residue was diluted with DCM (400 mL). The resulting mixture was poured into ice-water (1.20 L). The resulting mixture was extracted with DCM (200 mL). The organic layer was washed with saturated NaHCO; (300 mL) and brine (200 mL), then dried with MgSO₄ and filtered. The filtrate was concentrated to dryness to afford 2,4,6-trichloro-8-fluoroquinazoline (Int-K2). ¹H NMR (400 MHz, CDCl₃) δ 8.07 (t, J=2.0 Hz, 1H), 7.69-7.72 (m, 1H).

### Step C: tert-butyl 3-(2.6-dichloro-8-fluoroquinazolin-4-yl)-3,8-diazabicyclo[3.2.1]octane-8-carboxylate (Int-K3)

2,4,6-Trichloro-8-fluoroquinazoline (Int-K2) (30.0 g, 0.12 mol) was dissolved in DCM (150 mL) at 15-25° C. Triethylamine (36.2 g, 0.36 mol) was added to the mixture at 15-25° C. followed by tert-butyl 3.8-diazabicyclo[3.2.1]octane-8-carboxylate (25.3 g, 0.12 mol). The resulting mixture was stirred at 15-25° C. for 2 h. The reaction mixture was quenched with ice-water (120 mL) and the resulting mixture was stirred at 20-25° C. for 10 mins. The organic layer was isolated and dried with Na₂SO₄. The dried solution was filtered and concentrated under reduced pressure. The residue was triturated with EtOH (120 mL) at 20-25° C. for 16 h. tert-Butyl 3-(2,6-dichloro-8-fluoroquinazolin-4-yl)-3,8-diazabicyclo[3.2.1]octane-8-carboxylate (Int-K3) was obtained by filtration. ¹H NMR (400 MHz, CDCl₃) δ 7.60 (t, J=2.0 Hz, 1H), 7.44 (dd, J=9.2 Hz, 2.0 Hz, 1H), 4.39 (m, 4H), 3.64 (m, 2H), 1.95-1.97 (m, 2H), 1.73-1.75 (m, 2H), 1.53 (s, 9H).

### Intermediate M3: 7-bromo-2,4,6-trichloro-8-fluoroquinazoline (Int-M3)

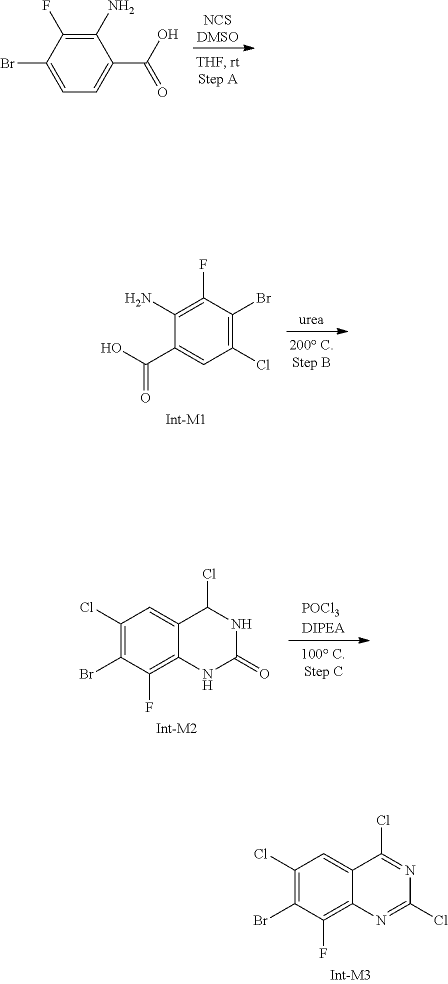

### Step A. 2-amino-4-bromo-5-chloro-3-fluorobenzoic acid (Int-M1)

Into a 10-L 4-necked round-bottom flask, were added 2-amino-4-bromo-3-fluorobenzoic acid (700 g, 2.99 mol), THF (7.0 L) and DMSO (93.5 g, 1.20 mol). This was followed by the addition of NCS (799 g, 5.98 mol) in several batches at 25° C. The resulting solution was stirred overnight at room temperature. The reaction was then quenched by the addition of 7 L of sat. NaCl (aq). The resulting solution was extracted with ethyl acetate (2 ×2 L) and the organic layer was washed with water (3×1 L), and dried over anhydrous sodium sulfate. The dried solution was filtered, and the filtrate was concentrated. The residue obtained was triturated in petroleum ether:ethyl acetate=10:1 (5 L) for 1 h. The solid was collected by filtration to afford 2-amino-4-bromo-5-chloro-3-fluorobenzoic acid (Int-M1) which was used directly in the next step without purification.

### Step B: 7-bromo-6-chloro-8-fluoroquinazoline-2,4(1H,3H)-dione (Int-M2)

A 2-L 4-necked round-bottom flask was charged with 2-amino-4-bromo-5-chloro-3-fluorobenzoic acid (Int-M1) (100 g, 372 mmol) and urea (112 g, 1.86 mol). The resulting solution was stirred for 2 h at 200° C. The reaction mixture was cooled to 25° C. and the reaction solidified. This reaction was repeated in 6 additional batches using the above conditions. The seven batches of reactions were combined and the bulk of the solid was ground to afford a crude mixture of 7-bromo-6-chloro-8-fluoroquinazoline-2,4(1H,3H)-dione (Int-M2) that was used directly in the next step without purification.

### Step C: 7-bromo-2,4,6-trichloro-8-fluoroquinazoline (Int-M3)

A 3-L 4-necked round-bottom flask was charged with 7-bromo-6-chloro-8-fluoroquinazoline-2,4(1H,3H)-dione (Int-M2) (290 g, 0.980 mol) and POCl₃ (1.51 kg, 9.86 mol). This was followed by the addition of DIEA (382 g, 2.96 mol) dropwise with stirring at 25 C. The resulting solution was stirred for 2 h at 100° C. This reaction was repeated in 2 additional batches using the above conditions. The three batches of reactions were combined and concentrated. The residue was purified by silica gel column eluted with petroleum ether:ethyl acetate=10:1. The crude product was slurried in petroleum ether:ethyl acetate =5:1(1 L). The solid was collected by filtration to afford 7-bromo-2,4,6-trichloro-8-fluoroquinazoline (Int-M3). ¹H NMR (300 MHz, CDCl₃, ppm): S 8.227 (s, 1H).

### Intermediate N7: 7-bromo-2,4-dichloro-6,8-difluoroquinazoline (Int-N7)

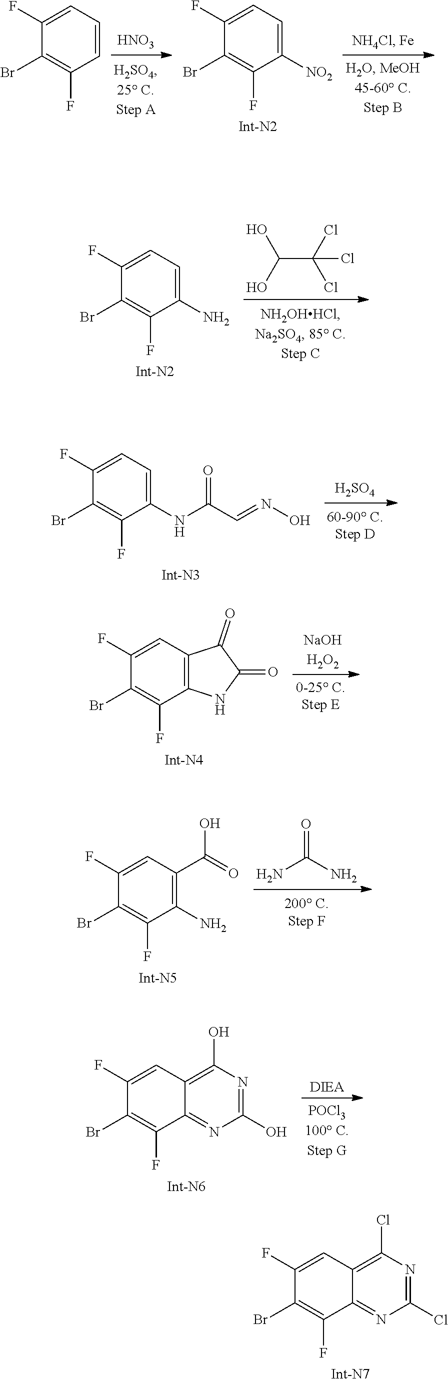

### Step A: 2-bromo-1,3-difluoro-4-nitrobenzene (Int-N1)

To a flask containing H₂SO₄(1.14 L) at 25° C. were added 2-bromo-1,3-difluorobenzene (567 g, 2.94 mol), followed by HNO₃ (284 g, 3.15 mol, 70% purity). Three reactions of the same scale were set up in parallel. The reaction mixtures were stirred for 15 minutes. The three reactions were then combined and added into ice water (10.0 L). The mixture was extracted with DCM (3.00 L×3) and washed with saturated NaHCO₃ (1.00 L×3). The organic layer was collected and dried with Na₂SO₄, filtered and concentrated to yield a crude residue. The residue was washed with petroleum ether (2.00 L) at 25° C. for 20 minutes and then filtered to obtain 2-bromo-1,3-difluoro-4-nitrobenzene (Int-N1). ¹H NMR: (400 MHz, CDCl₃) δ: 8.04-8.16 (m, 1H), 7.11-7.27 (m, 1H).

### Step B: 3-bromo-2,4-difluoroaniline (Int-N2)

To a flask containing MeOH (1.6 L) at 25° C. were added 2-bromo-1,3-difluoro-4-nitrobenzene (Int-N1) (320 g, 1.34 mol), followed by NH₄Cl (719 g, 13.4 mol) in H₂O (1.60 L). The mixture was warmed to 40° C. and Fe (300 g, 5.38 mol) was added portion-wise. Five reactions of the same scale were set up in parallel. The mixture was then stirred for 3 hours at 65° C. The five reactions were combined and added to ethyl acetate (7.00 L). The mixture was filtered and the filtrate collected. The filtrate was extracted with ethyl acetate (2.00 L×3) and washed with saturated NaHCO₃(1.50 L×3). The organic layer was collected and dried with Na₂SO₄, filtered and concentrated to yield 3-bromo-2,4-difluoroaniline (Int-N2) that was used in the next step without further purification. 1H NMR: (400 MHz, CDCl₃) S: 6.75-6,79 (m, 1H), 6.67-6,69 (m, 1H), 3.66 (s, 2H).

### Step C: (E)-N-(3-bromo-2,4-difluorophenyl)-2-(hydroxyimino)acetamide (Int-N3)

To a flask containing H₂O (1.6 L) at 25° C. were added Na₂SO₄ (922 g, 6.49 mol) and 2,2,2-trichloroethane-1,1-diol (131 g, 793 mmol), followed by a solution of 3-bromo-2,4-difluoroaniline (Int-N2) (150 g, 721 mmol) and conc. HCl (35.2 g, 965 mmol) in H₂O (400 mL) at 40° C. NH₂OH HCl (150 g, 2.16 mol) in H₂O (500 mL) was added. Eight reactions of the same scale were set up in parallel. The reaction mixture was then stirred for 2 hours at 85° C. The eight reactions were combined and added to ice water (10.0 L) and stirred for 10 minutes. The biphasic mixture was extracted with DCM (2.50 L×3) and washed with saturated NaHCO₃(2.00 L×3). The organic layer was dried with Na₂SO₄, filtered and concentrated to yield (E)-N-(3-bromo-2,4-difluorophenyl)-2-(hydroxyimino)acetamide (Int-N3) which was used in the next step without further purification. ¹H NMR (400 MHz, DMSO-d₆) δ: 12.34 (s, 1H), 10.0² (s, 1H), 7.75-7.80 (m, 1H), 7.70 (s, 1H), 7.27-7.32 (t, J=8.4 Hz, 1.6 Hz, 1H).

### Step D: 6-bromo-5,7-difluoroindoline-2,3-dione (Int-N4)

To a flask containing H₂SO₄ (3.00 L) at 60° C. under N₂ was added (E)-N-(3-bromo-2,4-difluorophenyl)-2-(hydroxyimino)acetamide (Int-N3) (200 g, 716 mmol). Five reactions of the same scale were set up in parallel. The reaction mixture was stirred at 90° C. for 3 hours. The five reactions were cooled, combined, and poured into ice (10.0 kg). The reaction mixture was filtered and the filtrate was dried with Na₂SO₄, filtered, and concentrated to obtain 6-bromo-5,7-difluoroindoline-2,3-dione (Int-N4). 1H NMR: (400 MHz, DMSO-d₆) δ: 11.69 (s, 1H), 7.50-7.52 (m, 1H).

### Step E: 2-amino-4-bromo-3,5-difluorobenzoic acid (Int-N5)

To a flask containing aqueous NaOH (2.00 M, 1.72 L) at 25° C. was added 6-bromo-5,7-difluoroindoline-2,3-dione (Int-N4) (100 g, 382 mmol). The reaction mixture was cooled to 0° C. and H₂O₂ was added (220 g, 1.94 mol, 30% purity). Five reactions of the same scale were set up in parallel. The reaction mixture was stirred at 0° C. for 30 minutes, then warmed to 25° C. and stirred for 16 hours. The five reactions were combined and poured into ice water (5(0) mL). The solution was acidified with concentrated HCl solution (500 mL). The precipitate was collected by filtration and dried in the air to obtain 2-amino-4-bromo-3,5-difluorobenzoic acid (Int-N5). ¹H NMR: (400 MHz, DMSO-d₆) S: 7.45-7.48 (m, 1H).

### Step F: 7-bromo-6,8-difluoroquinazoline-2,4-diol (Int-N6)

2-amino-4-bromo-3,5-difluorobenzoic acid (Int-N5) (65.0 g, 257 mmol) and urea (77.4 g, 1.29 mol) were combined and stirred at 200° C. for 2 hours. After cooling to 100° C., H₂O (200 mL) was added to triturate. The reaction mixture was then filtered and the solid was collected to yield 7-bromo-6,8-difluoroquinazoline-2,4-diol (Int-N6). ¹H NMR (400 MHz, DMSO-4) S: 11.25 (br. s, 2H), 7.59-7.62 (m, 1H)

### Step G: 7-bromo-2,4-dichloro-6,8-difluoroquinazoline (Int-N7)

DIEA (27.0 g, 209 mmol) was added portion wise to the mixture of 7-bromo-6,8-difluoroquinazoline-2,4-diol (Int-N6) (29.0 g, 104 mmol) in POCl₃ (192 g, 1.26 mol) at 25° C. The reaction mixture was stirred at 100° C. for 8 hours. The mixture was cooled to 20° C. and concentrated under reduced pressure at 40° C. The residue was purified by silica gel column chromatography (1000/1 to 10/1 petroleum ether/ethyl acetate) to obtain 7-bromo-2,4-dichloro-6,8-difluoroquinazoline (Int-N7). ¹H NMR (400 MHz, McOD) δ 8.04-8.09 (m, 1H).

### Intermediate O2: tert-butyl 3-(7-bromo-2-((1-((dimethylamino)methyl)cyclopropyl)methoxy)-6,8-difluoroquinazolin-4-yl)-3,8-diazabicyclo[3.2.1]octane-8-carboxylate (Int-O2)

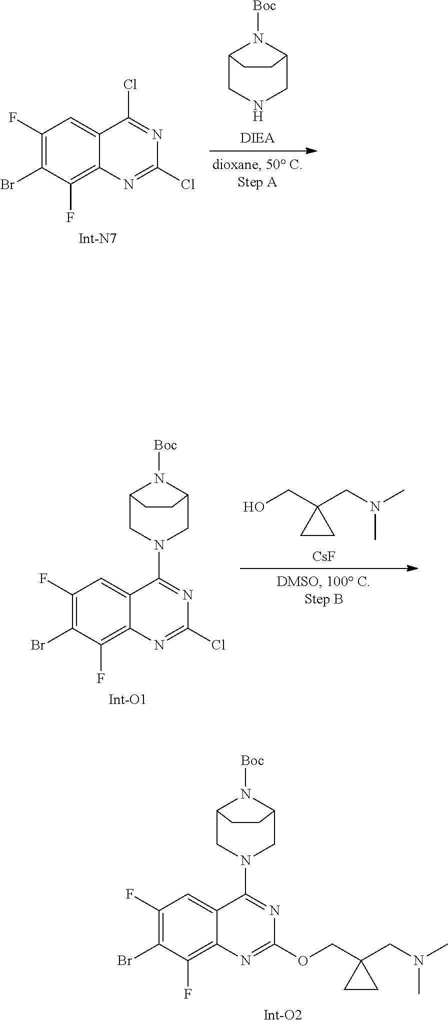

### Step A: tert-butyl 3-(7-bromo-2-chloro-6,8-difluoroquinazolin-4-yl)-3,8-diazabicyclo[3.2.1]octane-8-carboxylate (Int-O1)

To a solution of 7-bromo-2,4-dichloro-6,8-difluoroquinazoline (3.00 g, 9.08 mmol) and DIEA (7.93 mL, 45.4 mmol) in dioxane (12 mL) was added tert-butyl 3,8-diazabicyclo[3.2.1]octane-8-carboxylate (1.97 g, 9.26 mmol). The reaction mixture was stirred at 50° C. for 20 mm. The product mixture was evaporated to dryness. The residue obtained was purified by a silica gel column eluting from 0 to 30% ethyl acetate in hexanes to give tert-butyl 3-(7-bromo-2-chloro-6,8-difluoroquinazolin-4-yl)-3,8-diazabicyclo[3.2.1]octane-8-carboxylate (Int-O1). MS (ESI): m/z 489, 491 [M+H]⁺

### Step B: tert-butyl 3-(7-bromo-2-((1-((dimethylamino)methyl)cyclopropyl)methyl)-6,8-difluoroquinazolin-4-yl)-3,8-diazabicyclo[3.2.1]octane-8-carboxylate (Int-O2)

To a solution of(1-((dimethylamino)methyl)cyclopropyl)methanol (666 mg, 5.16 mmol) and tert-butyl 3-(7-bromo-2-chloro-6,8-difluoroquinazolin-4-yl)-3,8-diazabicyclo[3.2.1]octane-8-carboxylate (Int-O1) (1.01 g, 2.06 mmol) in DMSO (10.3 mL) was added cesium fluoride (1.25 g, 8.25 mmol). The reaction mixture was stirred at 100° C. The reaction mixture was allowed to cool to room temperature and partitioned between water and chloroform/isopropyl alcohol (3:1). After passing through a phase separator, the organic layer was evaporated to dryness. The residue obtained was purified by silica gel column chromatography (0 to 10% 7 N ammonia in methanol-DCM) to give tert-butyl 3-(7-bromo-2-((1-((dimethylamino)methyl)cyclopropyl)methoxy)-6,8-difluoroquinazolin-4-yl)-3,8-diazabicyclo[3.2.1]octane-8-carboxylate (Int-O2). MS (ESI): m/z 582, 584 [M+H]⁺.

The examples in the table below were synthesized via a reaction sequence similar to the one described above for Int-O2, where either Int-M3 or Int-N7 were used in combination with amines that are either commercially available or whose syntheses are found in the sections below and various alcohols that are either commercially available or whose syntheses are found in the sections below. When Int-M3 is used in combination with tert-butyl 3.8-diazabicyclo[3.2.1]octane-8-carboxylate, this will be referred to as Int-O1.

| | | | [M + H]⁺ Found |
|---|---|---|---|
| Int | Structure | Compound Name | or NMR Spectra |
| Int-P2 | 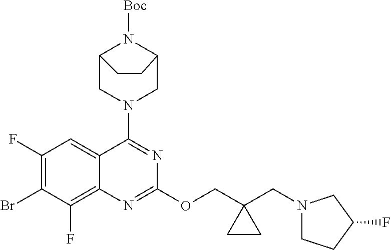 [SMILES: CC(C)(C)OC(=O)N1C2CCC1CN(c1nc(OCC3(CN4CC[C@@H](F)C4)CC3)nc3c(F)c(Br)c(F)cc13)C2] | tert-butyl 3-(7-bromo- 6,8-difluoro-2-((1- (((R)-3-fluoropyrrolidin-1-yl)methyl) cyclopropyl)methoxy)quinazolin- 4-yl)-3,8-diazabicyclo[3.2.1]octane-8- carboxylate | 626/628 |
| Int-Q2 | 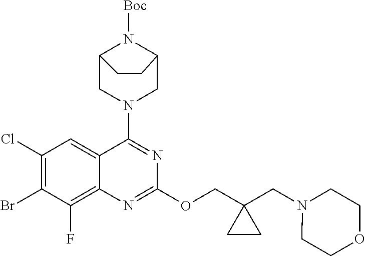 [SMILES: CC(C)(C)OC(=O)N1C2CCC1CN(c1nc(OCC3(CN4CCOCC4)CC3)nc3c(F)c(Br)c(Cl)cc13)C2] | tert-butyl 3-(7-bromo- 6-chloro-8-fluoro- 2-((1-(morpholinomethyl) cyclopropyl)methoxy) quinazolin-4-yl)-3,8- diazabicyclo[3.2.1] octane-8-carboxylate | 640/642 |
| Int-R2 | 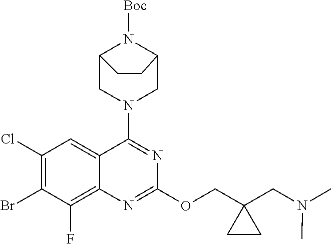 [SMILES: CN(C)CC1(COc2nc(N3CC4CCC(C3)N4C(=O)OC(C)(C)C)c3cc(Cl)c(Br)c(F)c3n2)CC1] | tert-butyl 3-(7-bromo-6-chloro-2-((1- ((dimethylamino)methyl)cyclopropyl) methoxy)-8-fluoroquinazolin-4- yl)-3,8-diazabicyclo[3.2.1] octane-8-carboxylate | 598/600 |
| Int-S2 | 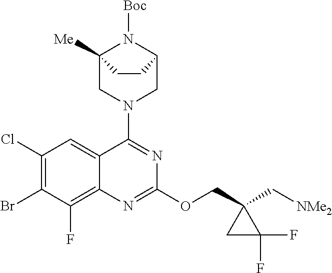 [SMILES: CN(C)C[C@@]1(COc2nc(N3C[C@H]4CC[C@@](C)(C3)N4C(=O)OC(C)(C)C)c3cc(Cl)c(Br)c(F)c3n2)CC1(F)F] | tert butyl (1S,5R)-3- (7-bromo-6-chloro-2- (((R)-1-((dimethylamino)methyl)-2,2- difluorocyclopropyl)methoxy)-8- fluoroquinazolin-4-yl)-1-methyl-3,8- diazabicyclo[3.2.1] octane-8-carboxylate | 648/650 |
| Int-T2 | 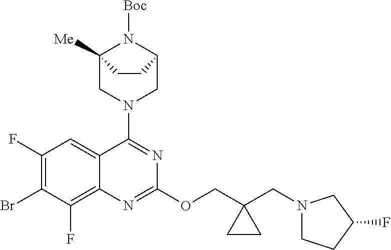 [SMILES: CC(C)(C)OC(=O)N1[C@@H]2CC[C@@]1(C)CN(c1nc(OCC3(CN4CC[C@@H](F)C4)CC3)nc3c(F)c(Br)c(F)cc13)C2] | tert-butyl (1S,5R)-3-(7-bromo-6,8- difluoro-2-((1-(((R)- 3-fluoropyrrolidin-1- yl)methyl)cyclopropyl)methoxy) quinazolin-4-yl)-1-methyl-3,8- diazabicyclo[3.2.1] octane-8-carboxylate | 640/642 |
| Int-U2 | 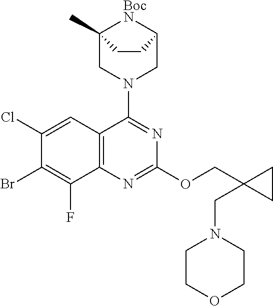 [SMILES: CC(C)(C)OC(=O)N1[C@@H]2CC[C@@]1(C)CN(c1nc(OCC3(CN4CCOCC4)CC3)nc3c(F)c(Br)c(Cl)cc13)C2] | tert-butyl (1S,5R)-3- (7-bromo-6-chloro- 8-fluoro-2-((1-(morpholinomethyl) cyclopropyl)methoxy) quinazolin-4-yl)-1-methyl-3,8- diazabicyclo[3.2.1] octane-8-carboxylate | ¹H NMR (500 MHz, CHLOROFORM-d) δ = 7.69 (d, J = 1.5 Hz, 1H), 4.41-4.35 (m, 3H), 4.28 (br d, J = 12.5 Hz, 1H), 4.13-4.10 (m, 1H), 3.65 (t, J = 4.4 Hz, 4H), 3.53 (br d, J = 12.5 Hz, 2H), 2.49- 2.38 (m, 6H), 2.10-1.87 (m, 3H), 1.63 (s, 2H), 1.59 (s, 2H), 1.52 (s, 9H), 0.70- 0.66 (m, 2H), 0.47-0.42 (m, 2H). |
| Int-UV2 | 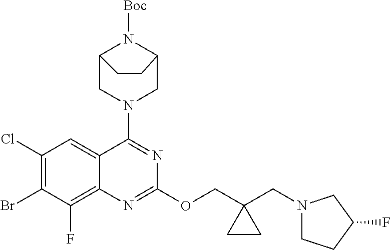 [SMILES: CC(C)(C)OC(=O)N1C2CCC1CN(c1nc(OCC3(CN4CC[C@@H](F)C4)CC3)nc3c(F)c(Br)c(Cl)cc13)C2] | tert-butyl 3-(7-bromo- 6-chloro-8-fluoro- 2-((1-(((R)-3-fluoropyrrolidin-1- yl)methyl)cyclopropyl) methoxy)quinazolin-4-yl)- 3,8-diazabicyclo[3.2.1]octane-8- carboxylate | 584/586 |

### Intermediate V4: 6-chloro-2-(1-((dimethylamino)methyl)cyclopropyl)methoxy)-8-fluoro-7-(3-(methoxymethoxy)naphthalen-1-yl)quinazolin-4-ol (Int-V4)

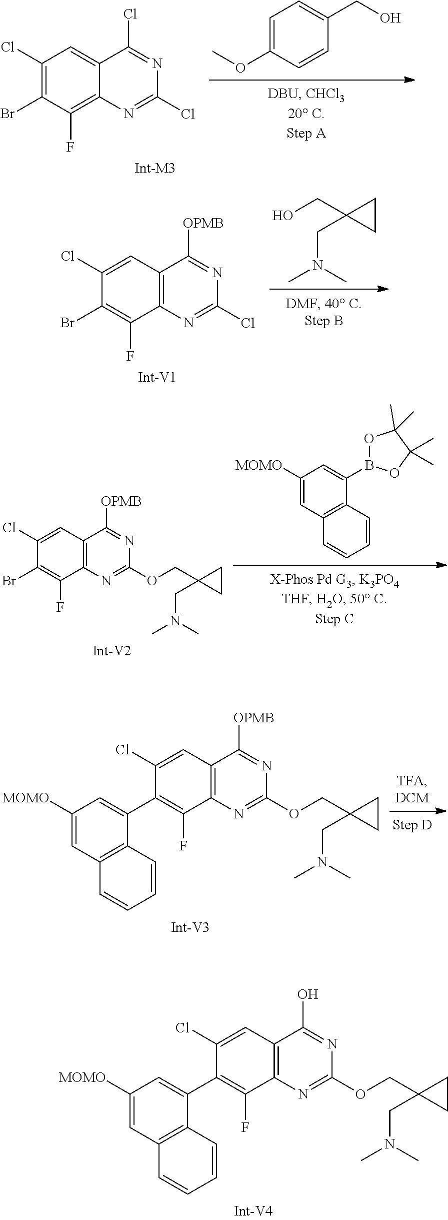

### Step A: 7-bromo-2,6-dichloro-8-fluoro-4-((4-methoxy benzyl)oxy)quinazoline (Int-yl)

To a stirred solution of 7-bromo-2,4,6-trichloro-8-fluoroquinazoline (Int-M3) (1 g, 3.03 mmol) in CHCl₃ (15 mL) were added (4-methoxyphenyl)methanol (0.544 g, 3.94 mmol), followed by DBU (0.684 mL, 4.54 mmol) at 20° C. After the addition was finished, the reaction was stirred at 20° C. for 16 h. The mixture was concentrated in vacuo, and the residue was purified by flash silica gel chromatography (Eluent of 0-50% DCM/Pet, ether gradient) to give 7-bromo-2,6-dichloro-8-fluoro-4-((4-methoxybenzyl)oxy)quinazoline (Int-yl). ¹H NMR (500 MHz, CDCl₃) δ 8.02 (d, J=2.0 Hz, 1H), 7.50-7.43 (M. 2H). 6.98-6,93 (m, 2H), 5.58 (s, 2H), 3.83 (s, 3H).

### Step B: 1-(1-((7-bromo-6-chloro-8-fluoro-4-((4-methoxybenzyl)oxy)quinazolin-2-yl)oxy)methylkyclopropyl)-N,N-dimethylmethanamine (Int-V2)

To a stirred solution of 7-bromo-2,6-dichloro-8-fluoro-4-((4-methoxybenzyl)oxy)quinazoline (Int-yl) (1 g, 2.31 mmol) in DMF (20 mL) was added (1-((dimethylamino)methyl)cyclopropyl)methanol (1,35 g, 10.4 mmol) at 20° C., and the mixture was stirred at 40° C. for 5 h. The mixture was dissolved in water (100 mL), and extracted with EtOAc (100 mL×2), the combined organic layers were dried over Na₂SO₄, filtered and concentrated in vacuo. The residue was purified by flash silica gel chromatography (eluent of 0-10% MeOH/DCM gradient) to afford 1-(1-(((7-bromo-6-chloro-8-fluoro-4-((4-methoxybenzyl)oxy)quinazolin-2-yl)oxy)methyl)cyclopropyl)-N,N-dimethylmethanamine (Int-V2). MS (ESI): m/z (M+H)+524, 526.

### Step C: 1-1-(((6-chloro-8-fluoro-4-((4-methoxybenzyl)oxy)-7-(3-(methoxymethoxy)naphthalen--yl)quinazolin-2-yl)oxy)methyl)cyclopropyl)-N,N-dimethylmethanamine (Int-V3)

To a mixture of 1-(1-(((7-bromo-6-chloro-8-fluoro-4-((4-methoxy benzyl)oxy)quinazolin-2-yl)oxy)methyl)cyclopropyl)-N,N-dimethylmethanamine (Int-V2) (760 mg, 1.45 mmol), 2-(3-(methoxymethoxy)naphthalen-1-yl)-4,4,5,5-tetramethyl-1,3,2-dioxaborolane (500 mg. 1.59 mmol) and potassium phosphate (2.90 mL, 4.34 mmol) (1.5M in water) in THF (20 mL) was added chloro(2-dicyclohexylphosphino-2′,4′,6′-triisopropyl-1,1′-biphenyl)[2-(2′-amino-1,1′-biphenyl)]palladium(II) (114 mg, 0.145 mmol) in a glove box, and the mixture was stirred at 50° C. for 2 h. The mixture was dissolved in water (80 mL) and extracted with EtOAc (80 mL×2). The combined organic layers were dried over Na₂SO₄, filtered and concentrated in vacuo, the residue was purified by flash silica gel chromatography (Eluent of 0-10% MeOH/DCM gradient) to afford 1-(1-(((6-chloro-8-fluoro-4-((4-methoxybenzyl)oxy)-7-(3-(methoxymethoxy)naphthalen-1-yl)quinazolin-2-yl)oxy)methyl)cyclopropyl)-N,N-dimethylmethanamine (Int-V3). MS (ESI): m/z (M+H)⁺ 632.

### Step D: 6-chloro-2-((1-((dimethylamino)methyl)cyclopropyl)methoxy)-8-fluoro-7-(3-(methoxymethoxy)naphthalen-1-yl)quinazolin-4-ol (Int-V4)

To a solution of 1-(1-(((6-chloro-8-fluoro-4-((4-methoxybenzyl)oxy)-7-(3-(methoxymethoxy)naphthalen-1-yl)quinazolin-2-yl)oxy)methyl)cyclopropyl)-N,N-dimethylmethanamine (Int-V3) (900 mg, 1.42 mmol) in DCM (25 mL) was added TFA (0.658 mL, 8.54 mmol), and the mixture was stirred at 20° C. for 30 min. The mixture was concentrated in vacuo, and the residue was purified by reverse phase HPLC (MeCN/water with 0.1% TFA modifier) to afford 6-chloro-2-((1-((dimethylamino)methyl)cyclopropyl)methoxy)-8-fluoro-7-(3-(methoxymethoxy)naphthalen-1-yl)quinazolin-4-ol (Int-V4). MS (ESI): m/z (M+H)⁺ 512.

Int-W4 in the table below was synthesized via the same synthetic route using Int-N7 as starting material.

| | | | [M + H]⁺ |
|---|---|---|---|
| Int | Structure | Compound Name | Found |
| Int-W4 | 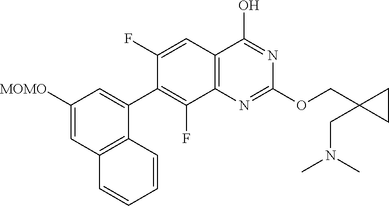 | 2-((1- ((dimethylamino)methyl) cyclopropyl)methoxy)- 6,8-difluoro-7-(3- (methoxymethoxy)naphthalen-1- yl)quinazolin-4-ol | 512 |
| Int-AI10 | 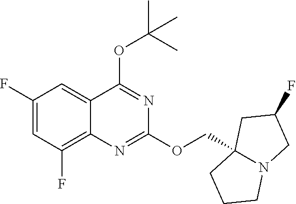 [SMILES: CC(C)(C)Oc1nc(OC[C@@]23CCCN2C[C@H](F)C3)nc2c(F)cc(F)cc12] | 4-(tert-butoxy)-6,8- difluoro-2-(((2R,7aS)-2- fluorotetrahydro-1H- pyrrolizin-7a(5H)- yl)methoxy)quinazoline | 396 |

### Intermediate X7: 8-(2-phenylpropan-2-yl)-3,8-diazabicyclo,[3,2,1]oct-6-ene (Int-X7)

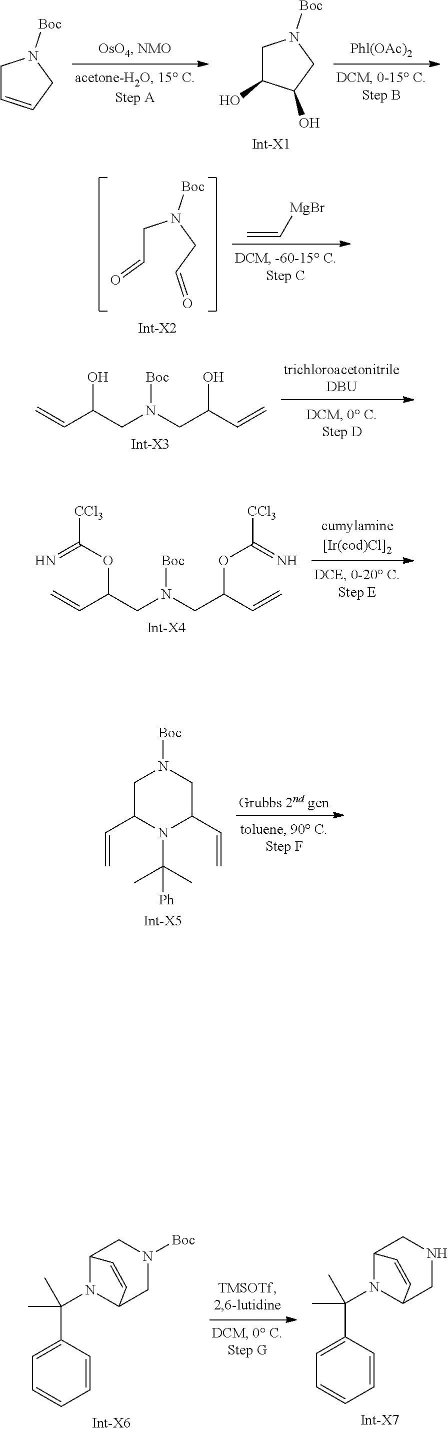

### Step A: tert-butyl cis-3,4-dihydroxypyrrolidine-1-carboxylate (Int-X1)

Osmium tetroxide (5.00 g, 19.7 mmol) and 4-methylmorpholine N-oxide (277 g, 2.36 mol) were added into water (990 mL). A solution of tert-butyl 2,5-dihydro-1H-pyrrole-1-carboxylate (333 g, 1.97 mol) in acetone (1.98 L) was added dropwise into the reaction vessel at 15° C. The reaction mixture was stirred at 15° C. for 16 h. This reaction was repeated in 2 additional batches using the above conditions. The three batches of reactions were combined and poured into saturated Na₂SO₃ aqueous solution (15.0 L). The quenched mixture was extracted with ethyl acetate (10.0 L×5). The organic layers were combined and dried over Na₂SO₄, filtered and concentrated to give the crude product. The crude material was purified by silica gel column chromatography (petroleum ether: ethyl acetate=20:1 to 0:1) to afford tert-butyl cis-3,4-dihydroxypyrrolidine-1-carboxylate (Int-X). ¹H NMR (CDCl₃, 400 MHz) S: 4.25 (d, J=3.6 Hz, 2H), 3.56-3.59 (m, 2H), 3.30-3.38 (m, 2H), 2.40-2.42 (m, 2H), 1.45 (s, 9H).

### Step B: tert-butyl bis(2-oxoethyl)carbamate (Int-X2)

tert-Butyl cis-3,4-dihydroxypyrrolidine-1-carboxylate (Int-X1) (100 g, 492 mmol) was dissolved in DCM (600 mL). (Diacetoxyiodo)benzene (190 g, 590 mmol) was added to the mixture at 0° C. The reaction mixture was stirred 15° C. for 1 h. The product mixture was concentrated to give tert-butyl bis(2-oxoethyl)carbamate (Int-X2), which was used in the next step without purification.

### Step C: tert-butyl bis(2-hydroxybut-3-en-1-yl)carbamate (Int-X3)

tert-Butyl bis(2-oxoethyl)carbamate (Int-X2) (99.0 g, 492 mmol) was dissolved to DCM (600 mL). The reaction mixture was cooled to −60° C. A 1 M solution of vinylmagnesium bromide (2.46 L, 2.46 mol) in THF was added dropwise to the mixture at −60° C. The reaction mixture was stirred 15° C. for 16 h. This reaction was repeated in 8 additional batches using the above conditions. The nine batches of reactions were combined and poured into ice cooled saturated NH₄Cl solution (30.0 L). The resulting mixture was extracted with ethyl acetate (5.00 L×2). The organic layers were combined and washed with brine (15.0 L). The organic layer was dried over Na₂SO₄, filtered and concentrated under reduced pressure to dryness. The residue was purified by silica gel column chromatography (petroleum ether: ethyl acetate=30: 1 to 1: 1) to afford tert-butyl bis(2-hydroxybut-3-en-1-yl)carbamate (Int-X3). ¹H NMR (400 MHz, CDCl₃) δ 5.70-5.97 (m, 2H), 5.23-5.46 (m, 2H). 5.16 (br d, J=10.0 Hz, 2H), 4.274.61 (m, 2H), 3.12-3.76 (m, 5H), 2.74-3.06 (m, 1H), 1.43-1.48 (m, 9H).

### Step D: ((tert-butoxycarbonyl)azanedivl)bis(but-3-ene-1,2-diyl) bis(2,2,2-trichloroacetimidate) (Int-X4)

tert-Butyl bis(2-hydroxybut-3-en-1-yl)carbamate (Int-X3) (215 g, 836 mmol) was dissolved in DCM (1.29 L). The reaction mixture was cooled down to 0° C. Trichloroacetonitrile (362 g, 2.51 mol) was added into the mixture. DBU (63.6 g, 418 mmol) was added into the reaction mixture at 0 C. The reaction mixture was stirred at 0° C. for 1 h. This reaction was repeated in 2 additional batches using the above conditions. The three batches of reactions were combined and concentrated. The residue obtained was purified by silica gel column chromatography (petroleum ether: ethyl acetate=8:1 to 5:1) to afford ((tert-butoxycarbonyl)azanediyl)bis(but-3-ene-1,2-diyl) bis(2,2,2-trichloroacetimidate) (Int-X4). ¹H NMR (400 MHz, CDCl₃) δ 8.37 (s, 2H), 5.72-5.95 (m, 2H), 5.53-5.71 (m, 2H). 5.41 (br dd, J=17.2, 8.4 Hz, 2H), 5.19-5.31 (m, 2H), 3.73-3.88 (m, 1H), 3.54-3.72 (m, 2H), 3.45 (ddd, J=18,4, 14.8, 8.8 Hz, 1H), 1.47 (d, J=3.2 Hz, 9H). Step E: tert-butyl 4-(2-phenylpropan-2-yl)-3,5-divinylniperazine-1-carboxylate (Int-X5)

Cumylamine (40.9 g, 302 mmol) was dissolved in DCE (9(X) mL). [Ir(cod)Cl]₂ (9.22 g, 13.7 mmol) was added into the reaction mixture. The reaction vessel was purged with N₂ three times. A solution ((tert-butoxycarbonyl)azanediyl)bis(but-3-ene-1,2-diyl) bis(2,2,2-trichloroacetimidate) (Int-X4) (150 g, 275 mmol) in DCE (900 mL) was added dropwise into the reaction mixture at 0° C. under N₂. The reaction vessel was purged with N₂ three more times. The reaction mixture was stirred at 20° C. for 16 h. This reaction was repeated in 6 additional batches using the above conditions. The seven batches of reactions were combined and concentrated. The residue obtained was purified by silica gel column chromatography (petroleum ether: ethyl acetate=20: 1 to 5:1) to afford tert-butyl 4-(2-phenylpropan-2-yl)-3,5-divinylpiperazine-1-carboxylate (Int-X5). ¹H NMR (400 MHz, CDCl₃) δ 7.53-7.35 (m, 5H), 5.91-6.04 (m, 2H), 4.93-5.17 (m, 4H), 3.10-3.82 (m, 6H), 1.41-1.49 (m, 15H).

### Step F: Tert-butyl 8-(2-phenylpropan-2-yl)-3,8-diazabicyclo[3.2.1]oct-6-ene-3-carboxylate (Int-X6)

Tert-Butyl 4-(2-phenylpropan-2-yl)-3,5-divinylpiperazine-1-carboxylate (Int-X5) (80.0 g, 224 mmol) was dissolved in toluene (3.2 L) under N2. Grubbs 2^{nd} generation catalyst (7.62 g, 8.98 mmol) was added into the mixture under N2. The reaction vessel was purged with N₂ three times. The reaction mixture was stirred at 90° C. for 12 h under N₂. This reaction was repeated in 5 additional batches using the above conditions. The six batches of reactions were combined and concentrated. The residue obtained was purified by column chromatography (SiO₂, petroleum ether: ethyl acetate=20: 1 to 5: 1) to give the crude product. The crude product was triturated with petroleum ether (2.00 L), tert-Butyl 8-(2-phenylpropan-2-yl)-3,8-diazabicyclo[3.2.1]oct-6-ene-3-carboxylate (Int-X6) was collected by filtration. ¹H NMR (400 MHz, CDCl₃) δ 7.55-7.63 (m, 2H), 7.32 (t, J=7.6 Hz, 2H), 7.18-7.26 (m, 1H), 5.93-6,03 (m, 2H), 3.58-3.68 (m, 2H), 3.44-3.56 (m, 2H), 3.10 (ddd, J=18.0, 11.6, 2.4 Hz, 2H), 1.38-1.47 (m, 9H), 1.26 (d, J=6.2 Hz, 6H).

### Step G: 8-(2-phenylpropan-2-yl)-3,8-diazabicyclo[3.2.1]oct-6-ene (Int-X7)

Tert-Butyl 8-(2-phenylpropan-2-yl)-3,8-diazabicyclo[3.2.1]oct-6-ene-3-carboxylate (Int-X6) (5 g, 15.2 mmol) was added in a 250 mL round bottom flask. The reaction vessel was evacuated and backfilled with a balloon of nitrogen three times. DCM (38.1 ml) and 2,6-dimethylpyridine (10.6 mL, 91 mmol) were added into the reaction vessel. The resulting mixture was cooled down to 0° C. Trimethylsilyl trifluoromethanesulfonate (11.0 mL, 60.9 mmol) was added dropwise into the reaction vessel. The resulting mixture was stirred for 1 h at 0° C. The product mixture was quenched by carefully adding saturated sodium bicarbonate aqueous solution (15 mL) dropwise. Vigorous gas evolution was observed within the first 0.5 mL addition of the solution. The quenched product mixture was extracted three times with DCM (3×100 mL). The organic layers were dried and the dried solution was filtered. The filtrate was concentrated to dryness to yield 8-(2-phenylpropan-2-yl)-3,8-diazabicyclo[3.2.1]oct-6-ene (Int-X7), which was used in subsequent steps without further purification. MS (ESI): m/z (M+H)⁺ 229.

### Intermediate Y3-1-1: (1S,5S,6R)-8-(2-phenylpropan-2-yl)-3,8-diazabicyclo[3.2.1]octan-6-ol (Int-Y3-1-1)

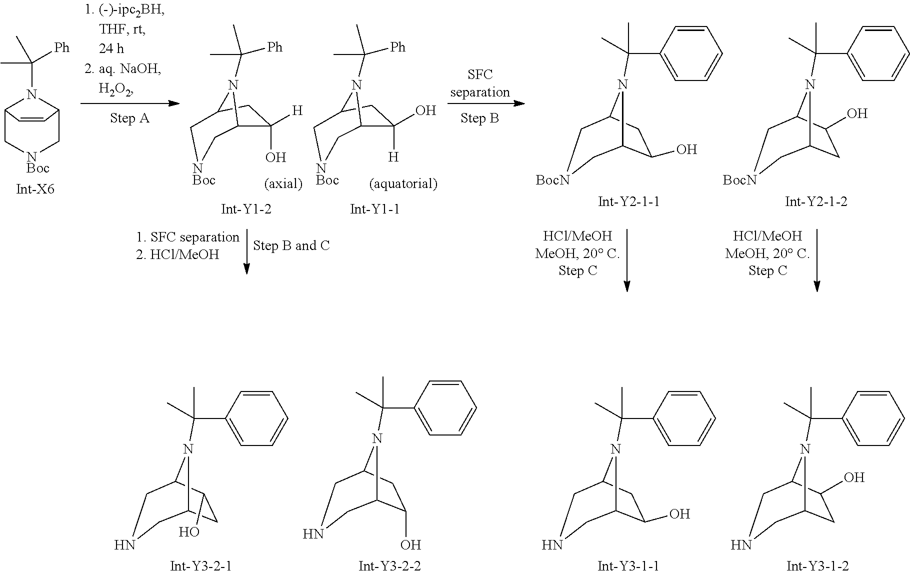

### Step A: tert-butyl 6-hydroxy-8(2-phenylpropan-2-yl 3.8-diazabicyclo[3.2.1]octane-3-carboxylate (Int-Y1-1)

To a solution of tert-butyl 8-(2-phenylpropan-2-yl)-3,8-diazabicyclo[3.2.1]oct-6-ene-3-carboxylate (Int-X6) (10 g, 30.4 mmol) in THF (10 mL) was added (−)-diisopinocampheyl borane (39.6 mL, 39.6 mmol) at 25° C. under N2 atmophere, and the mixture was stirred at 25° C. for 24 h. 3 M aqueous NaOH (30.4 mL, 91 mmol) was very slowly added to the mixture. After the gas evolution ceased, H₂O₂(16.0 mL, 182 mmol, 35% in water) was added into the reaction vessel in one portion. The resulting mixture was stirred at 50° C. for 2 h. The combined reaction mixture was quenched with saturated Na₂SO₃ solution (50 mL), and the resulting mixture was partitioned between EtOAc (300 mL×3) and water (80 mL). The combined organic phase was dried over anhydrous Na₂SO₄, filtered and concentrated in vacuo. The residue was purified by flash silica gel Chromatography (Pet, ether/EtOAc=3/1) to give tert-butyl 6-hydroxy-8-(2-phenylpropan-2-yl)-3,8-diazabicyclo[3.2.1]octane-3-carboxylate (Int-Y1-1) as the first eluting isomer. MS (ESI): m/z (M+H)⁺ 347. And tert-butyl 6-hydroxy-8-(2-phenylpropan-2-yl)-3,8-diazabicyclo[3.2.1]octane-3-carboxylate (Int-Y1-2) as the second eluting isomer. MS (EST): m/z (M+H)⁺ 347.

### Step B: tert-butyl 6-hydroxy-8-(2-phenylpropan-2-yl)-3,8-diazabicyclo[3.2.1]octane-3-carboxylate (Int-Y2-1-1 and Int-Y2-1-2)

The racemic mixture of tert-butyl 6-hydroxy-8-(2-phenylpropan-2-yl)-3,8-diazabicyclo[3.2.1]octane-3-carboxylate (Int-Y1-1) (20 g, 57.7 mmol) was separated by preparative SFC (Column A; Condition: 0.1% NH₄OH in EtOH) to afford tert-butyl (1S,5S,6R)-6-hydroxy-8-(2-phenylpropan-2-yl)-3,8-diazabicyclo[3.2.1]octane-3-carboxylate (Int-Y2-1-1, the first eluting isomer) and tert-butyl (1R,5R,6S)-6-hydroxy-8-(2-phenylpropan-2-yl)-3,8-diazabicyclo[3.2.1]octane-3-carboxylate (Int-Y2-1-2, the second eluting isomer). MS (ESI): m/z (M+H)⁺ 347.

### Step C: (1S,5S,6R)-8-(2-phenvlpropan-2-yl)-3,8-diazabicyclo[3.2.1]octan-6-ol (Int-Y3-1-1)

To a mixture of tert-butyl (1S, 5S, 6R)-6-hydroxy-8-(2-phenylpropan-2-yl)-3,8-diazabicyclo[3.2.1]octane-3-carboxylate (Int-Y2-1-1) (150 mg, 0.433 mmol) in MeOH (2 mL) was added HCl/MeOH (4M) (6 mL), and the mixture was stirred at 20° C. for 1.5 h. The mixture was concentrated in vacuo to afford (1S,5S,6R)-8-(2-phenylpropan-2-yl)-3,8-diazabicyclo[3.2.1]octan-6-ol (Int-Y3-1-1). MS (ESI): m/z (M+H)⁺ 247. The absolute stereochemistry was confirmed via Mosher ester analysis.

### Intermediate Y3-1-2: (1R,5R,6S)-8-(2-pnhenylnronan-2-yl)-3,8-diazabicyclo[3.2.1]octan-6-ol

This intermediate was made via the same route as above, using Int-Y2-1-2. MS (ESI): m/z (M+H)⁺ 247. The absolute stereochemistry was confirmed via Mosher ester analysis.

### Intermediate Y3-2-1: (1R,5R,6R)-8-(2-phenylpropan-2-yl)-3,8-diazabicyclo[3.2.1]octan-6-ol

This intermediate was made via the same route as above, using Int-Y1-2, followed by SFC separation (Column A; Conditions: 0.1% NH₄OH in EtOH) and Boc-deprotection to yield (1R,5R,6R)-8-(2-phenylpropan-2-yl)-3,8-diazabicyclo[3.2.1]octan-6-ol (Int Y3-2-1, Peak 1). MS (EST): m/z (M+H)⁺ 247. The absolute stereochemistry was confirmed via Mosher ester analysis.

### Intermediate Y3-2-2: (1S,5S,6S)-8-(2-phenylpropan-2-yl)-3,8-diazabicyclo[3.2.1]octan-6-ol

This intermediate was made via the same route as above, using Int-Y1-2, followed by SFC separation (Column A; Conditions: 0.1% NH₄OH in EtOH) and Boc-deprotection to yield (1S,5S,6S)-8-(2-phenylpropan-2-yl)-3,8-diazabicyclo[3.2.1]octan-6-ol (Int Y3-2-2, Peak 2). MS (EST): m/z (M+H)⁺ 247. The absolute stereochemistry was confirmed via Mosher ester analysis.

### Intermediate Z2: 6-methoxy-8-(2-phenylpropan-2-yl)-3, 8-diazabicyclo [3.2.1]octane (Int-Z2)

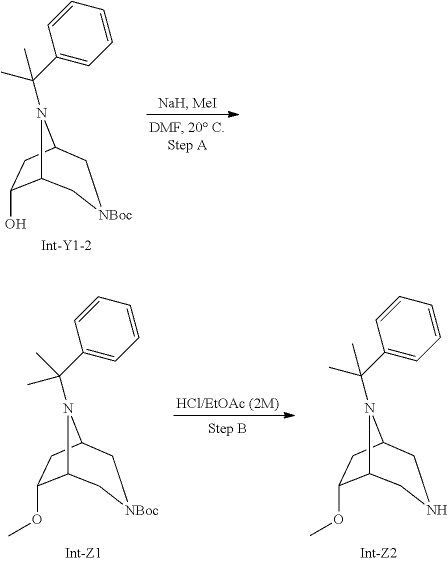

### Step A: tert-butyl 6-methoxy-8-(2-phenylpropan-2-yl)-3,8-diazabicyclo[3.2.1]octane-3-carboxylate (Int-Z1)

To a solution of tert-butyl 6-hydroxy-8-(2-phenylpropan-2-yl)-3,8-diazabicyclo[3.2.1]octane-3-carboxylate (Int-Y1-2) (200 mg, 0.577 mmol) in DMF (2 mL) was added NaH (69.3 mg, 1.732 mmol) (60% in mineral oil) at 20° C., and the reaction was stirred at 20° C. for 5 min. Then Mel (0.108 mL, 1.73 mmol) was added at 20° C. and the mixture was stirred at 20° C. for 2 h. The reaction mixture was quenched with water (2 mL), and extracted with EtOAc (10 mL×2). The organic layer was dried over Na₂SO₄ and filtered, and the filtrate was concentrated under reduced pressure to give a solid, which was purified by flash silica gel chromatography (Eluent of 0˜20% EtOAc/Pet, ether gradient) to give tert-butyl 6-methoxy-8-(2-phenylpropan-2-yl)-3,8-diazabicyclo[3.2.1]octane-3-carboxylate (Int-Z1). MS (ES): m/z (M+H), 361.

### Step B: 6-methoxy-8-(2-phenylpropan-2-yl)-3, 8-diazabicyclo [3,2,1]octane (Int-Z2)

To a solution of EtOAc (1 mL) and HCl/EtOAc (1 mL, 4 M) was added tert-butyl 6-methoxy-8-(2-phenylpropan-2-yl)-3,8-diazabicyclo[3.2.1]octane-3-carboxylate (Int-Z1) (110 mg, 0.305 mmol) at 20° C. The reaction was stirred at 20° C. for 2 h. The mixture was quenched with saturated NH₄Cl (10 mL) and extracted with EtOAc (10 mL×3), dried over Na₂SO₄, and filtered. The filtrate was evaporated under reduced pressure. The crude product was purified by Prep-TLC (DCM: MeOH=10:1) to give 6-methoxy-842-phenylpropan-2-yl)-3,8-diazabicyclo [3,2,1]octane (Int-Z2) as a racemic mixture. MS (ESI): m/z (M+H)_{f}261.

### Intermediate AA2: 6-methoxy-842-phenylpropan-2-yl)-3, 8-diazabicyclo [3,2,1]octane (Int-AA2)

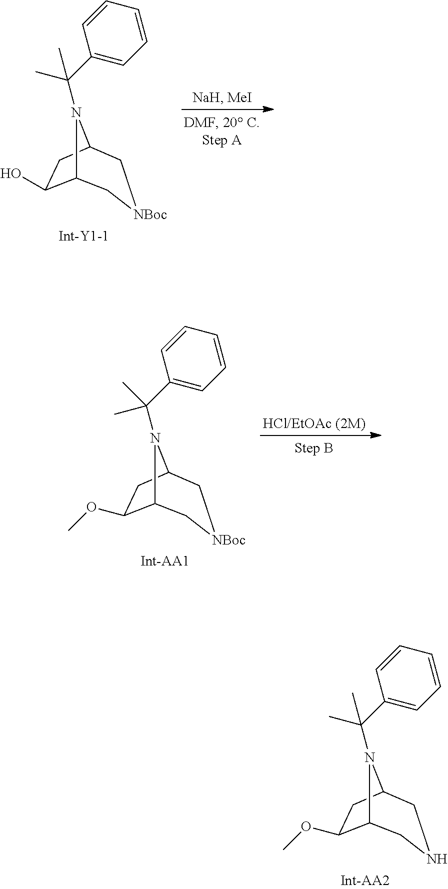

This racemic intermediate was made via the same route to make Int-Z2 as above, using Int-Y1-1 as the starting material. MS (ESI): m/z (M+H)⁺ 261.

### Intermediate BB4-1 and BB4-2: (1R,5S,6R)-8-(2-phenylpropan-2-yl)-3,8-diazabicyclo[3.2.1]octane-6-carbonitrile and (1S,5R,6S)-8-(2-phenylpropan-2-yl)-3,8-diazabicyclo[3.2.1]octane-6-carbonitrile (Int-BB4-1 and Int-BB4-2)

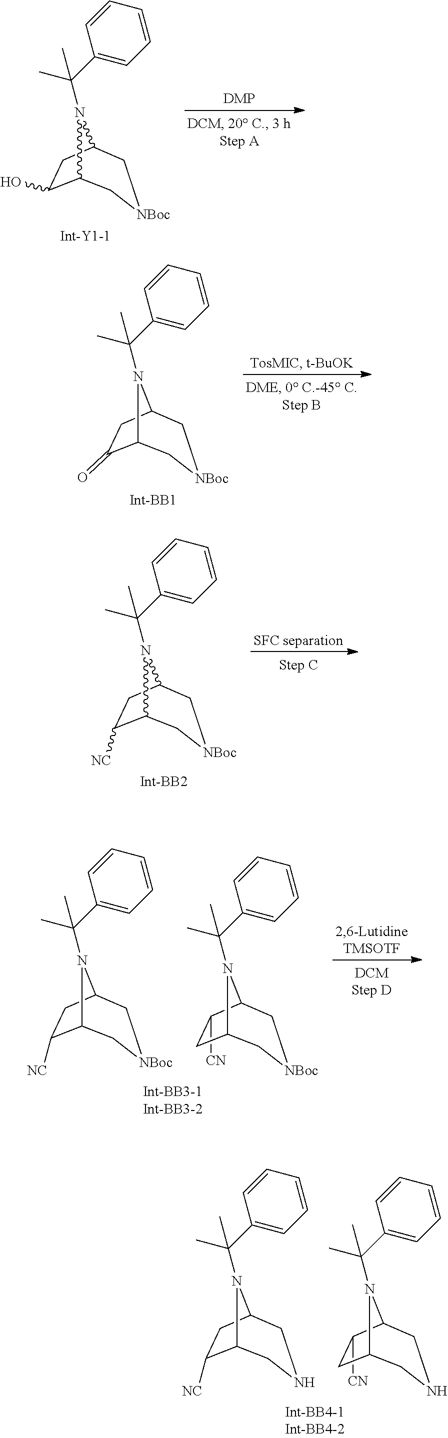

### Step A: tert-butyl 6-oxo-8-(2-phenylpropan-2-yl)-3,8-diazabicyclo[3.2.1]octane-3-carboxylate (Int-BB1)

To a solution of tert-butyl 6-hydroxy-8-(2-phenylpropan-2-yl)-3,8-diazabicyclo[3.2.1]octane-3-carboxylate (Int-Y1-1) (700 mg, 2.02 mmol) in DCM (15 mL) was added Dess-Martin periodinane (DMP) (1.29 g, 3.03 mmol) at 20° C. and the mixture was stirred at 20° C. for 2 h. The reaction mixture was partitioned between DCM (10 mL) and saturated aqueous NaHCO₃(8 mL). The organic phase was dried over anhydrous Na₂SO₄, filtered and concentrated in vacuo. The residue was purified by flash silica gel chromatography (Eluent of 0 to 15% EtOAc/Pet, ether gradient) to give tert-butyl 6-oxo-8-(2-phenylpropan-2-yl)-3,8-diazabicyclo[3.2.1]octane-3-carboxylate (Int-BB1). MS (ESI): m/z (M+H)⁺ 345.

### Step B: tert-butyl 6-cyano-8-(2-phenylpropan-2-yl)-3,8-diazabicyclo[3.2.1]octane-3-carboxy late (Int-BB2)

To a solution of potassium 2-methylpropan-2-olate (1091 mg, 9.73 mmol) in DME (10 mL) was added 1-((isocyanomethyl)sulfonyl)-4-methylbenzene (760 mg, 3.89 mmol) at 0° C. The mixture was stirred at 0° C. for 1 h, and then tert-butyl 6-oxo-8-(2-phenylpropan-2-yl)-3,8-diazabicyclo[3.2.1]octane-3-carboxylate (Int-BB1) (670 mg, 1.945 mmol) dissolved in DME (3 mL) was added at 0° C. The reaction was stirred at 45° C. for 2 h. The reaction mixture was quenched with saturated NH₄Cl (10 mL), and extracted with EtOAc (3×20 mL), and the organic layer was dried over sodium sulfate and evaporated under reduced pressure. The crude residue was purified by flash silica gel chromatography (Eluent of 0 to 35% EtOAc/Pet, ether gradient) to give tert-butyl 6-cyano-8-(2-phenylpropan-2-yl)-3,8-diazabicyclo[3.2.1]octane-3-carboxylate (Int-BB2). MS (ESI): m/z (M+H)⁺ 356.

### Step C: tert-butyl (1R5S,6R)-6-cyano-8-(2-phenylpropan-2-yl)-3,8-diazabicyclo[3.2.1]octane-3-carboxylate and tert-butyl (S,5R,6S)-6-cyano-8-(2-phenylpropan-2-yl)-3,8-diazabicyclo[3.2.1]octane-3-carboxylate (Int-BB3-1 and Int-BB3-2)

The racemic tert-butyl 6-cyano-8-(2-phenylpropan-2-yl)-3,8-diazabicyclo[3.2.1]octane-3-carboxylate (Int-BB2) (400 mg) was separated by preparative SFC (Column A; Condition: 0.1% NH₄OH in EtOH) to give Int-BB3-1 (the first eluting isomer). MS (ESI): m/z (M+H)⁺ 356. And Int-BB3-2 (the second eluting isomer). MS (ESI): m/z (M+H)⁺ 356.

### Step D: (1R,5S,6R) or (0S,5R,6S)-8-(2-phenylpropan-2-yl)-3,8-diazabicyclo[3.2.1]octane-6-carbonitrile (Int-BB4-1)

To a solution of Int-BB3-1 (80 mg, 0.225 mmol) in DCM (4 mL) was added 2,6-dimethylpyridine (0.157 mL, 1.35 mmol) and trimethylsilyl trifluoromethanesulfonate (0.163 mL, 0.900 mmol) at 0° C., and the mixture was stirred at 0° C. for 1 h. The reaction mixture was quenched with NaHCO₃and extracted with DCM (3×15 mL). The organic layer was dried over Na₂SO₄, filtered and the solvent was evaporated under reduced pressure to give crude product, which was purified by Prep-TLC (Pet, ether: EtOAc=1:1) to give Int-BB4-1. MS (ESI): m/z (M+H)₊ 256.

Int-BB4-2 was prepared in a similar fashion from Int-BB3-2.

### Intermediate CC5-1 and CCS-2: benzyl (1R,5S,6R)-6-methyl-3,8-diazabicyclo[3.2.1]octane-8-carboxylate and benzyl (1S,5R,6S)-6-methyl-3,8-diazabicyclo[3.2.1]octane-8-carboxylate (Int-CC5-1 and Int-CC5-2)

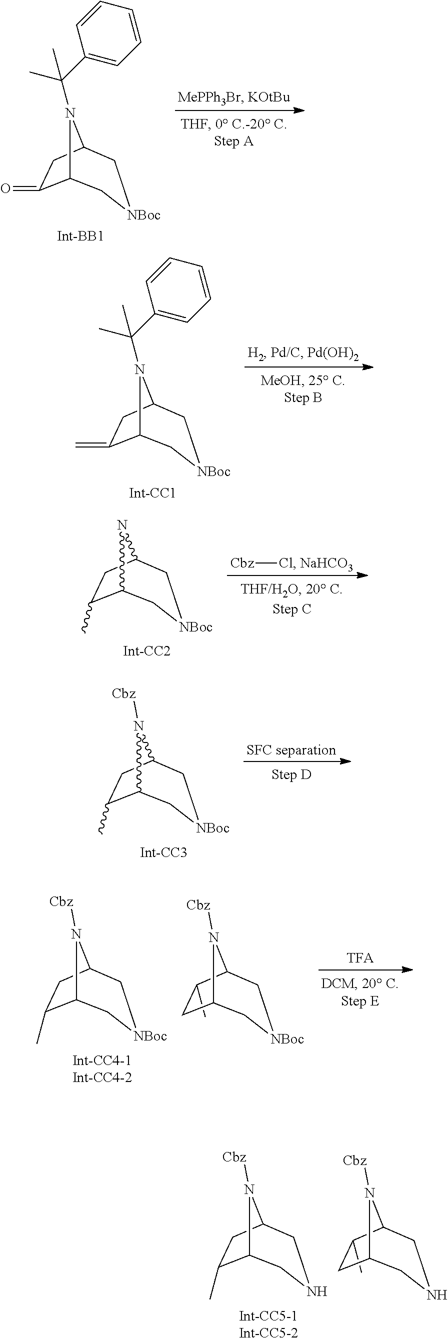

### Step A; tert-butyl 6-methylene-842-phenylpropan-2-yl)-3,8-diazabicyclo[3.2.1]octane-3-carboxylate (Int-CC1)

To a solution of methyltriphenylphosphonium bromide (3.42 g, 9.58 mmol) in THF (30 mL) was added potassium tert-butoxide (1.075 g, 9.58 mmol) at 0° C. and stirred at 20° C. for 15 min, then tert-butyl 6-oxo-8-(2-phenylpropan-2-yl)-3,8-diazabicyclo[3.2.1]octane-3-carboylate (Int-BB1) (1.1 g, 3.19 mmol) in THF (10 mL) was added into the reaction. The resulting mixture was stirred at 20° C. for 2 h. The reaction mixture was partitioned between EtOAc (30 mL×2) and water (20 mL). The combined organic phase was dried over anhydrous Na₂SO₄, filtered and concentrated in vacuo. The residue was purified by flash silica gel chromatography (Eluent of 0 to 15% EtOAc/Pet, ether gradient) to give tert-butyl 6-methylene-8-(2-phenylpropan-2-yl)-3,8-diazabicyclo[3.2.1]octane-3-carboxylate (Int-CC1). MS (EST): m/z (M+H)⁺ 343.

### Step B: tert-butyl 6-methyl-3,8-diazabicyclo[3.2.1]octane-3-carboxylate (Int-CC2)

To a solution of tert-butyl 6-methylene-8-(2-phenylpropan-2-yl)-3,8-diazabicyclo[3.2.1]octane-3-carboxylate (Int-CC1) (800 mg, 2.34 mmol) in MeOH (20 mL) was added Pd/C (8 mL, 2.34 mmol) and palladium hydroxide on carbon (5 mL, 2.34 mmol) at 25° C. under H₂ (15 psi) atmosphere. The mixture was stirred at 25° C. for 16 h. The mixture was filtered and the solvent was evaporated under reduced pressure to give the crude product tert-butyl 6-methyl-3,8-diazabicyclo[3.2.1]octane-3-carboxylate (Int-CC2) which was used in the subsequent step without further purification. MS (ESI): m/z (M+H)⁺ 227.

### Step C: 8-benzyl 3-(tert-butyl) 6-methyl-3,8-diazabicyclo[3.2.1]octane-3,8-dicarboxylate (Int-CC3)

To a solution of tert-butyl 6-methyl-3,8-diazabicyclo[3.2.1]octane-3-carboxylate (Int-CC2) (0.42 g, 1.86 mmol) in THF (8 mL) and water (2 mL) was added NaHCO₃ (0.468 g, 5.57 mmol) and Cbz-Cl (0.530 mL, 3.71 mmol) at 25° C.; the mixture was stirred at 25° C. for 2 h. The reaction mixture was partitioned between EtOAc (10 mL×2) and water (5 mL). The combined organic phase was dried over anhydrous Na₂SO₄, filtered and concentrated in vacuo. The residue was purified by reverse phase prep-HPLC (MeCN/water with 0.1% TFA modifier) to give 8-benzyl 3-(tert-butyl) 6-methyl-3,8-diazabicyclo[3.2.1]octane-3,8-dicarboxylate (Int-CC3) as the axial isomer. MS (ESI): m/z (M+H)⁺ 361.

### Step D: 8-benzyl 3-(tert-butyl) (1R,5S,6R)-6-methyl-3,8-diazabicyclo[3.2.1]octane-3,8-dicarboxylate and 8-benzyl 3-(tert-butyl) (1S,5R,6S)-6-methyl-3,8-diazabicyclo[3.2.1]octane-3,8-dicarboxylate (Int-CC4-2 and Int-C -2)

A mixture of 8-benzyl 3-(tert-butyl) 6-methyl-3,8-diazabicyclo[3.2.1]octane-3,8-dicarboxylate (Int-CC3) (650 mg. 1.803 mmol) was separated by SFC (Column D: Condition: 0.1% NH₄OH/IPA) to give Int-CC4-1 as the first eluting isomer. MS (ESI): m z (M+H)⁺ 361. And 8-benzyl 3-(tert-butyl) (1S,5R,6S)-6-methyl-3,8-diazabicyclo[3.2.1]octane-3,8-dicarboxylate (Int-CC4-2) as the second eluting isomer. MS (ESI): m/z (M+H)⁺ 361. Absolute stereochemistry unknown.

### Step E: (1S,5R,6S) or (1R,5S,6R)-benzyl 6-methyl-3,8-diazabicyclo[3.2.1]octane-8-carboxylate (Int-CC5-2)

A solution of 8-benzyl 3-(tert-butyl) 6-methyl-3,8-diazabicyclo[3.2.1]octane-3,8-dicarboxylate (Int-CC4-2) (80 mg, 0.222 mmol) in HCl/dioxane (1 mL, 4.0 mmol, 4 N) was stirred at 20° C. for 0.5 h. The mixture was concentrated in vacuo to afford crude Int-CC5-2. MS (ESI): m z (M+H)⁺ 261.

### Intermediate DD2-1: 6-methyl-8-(2-2-phenylorovan-2-yl)-3,8-diazabicyclo[3.2.1]octan-6-ol (Int-DD2-1)

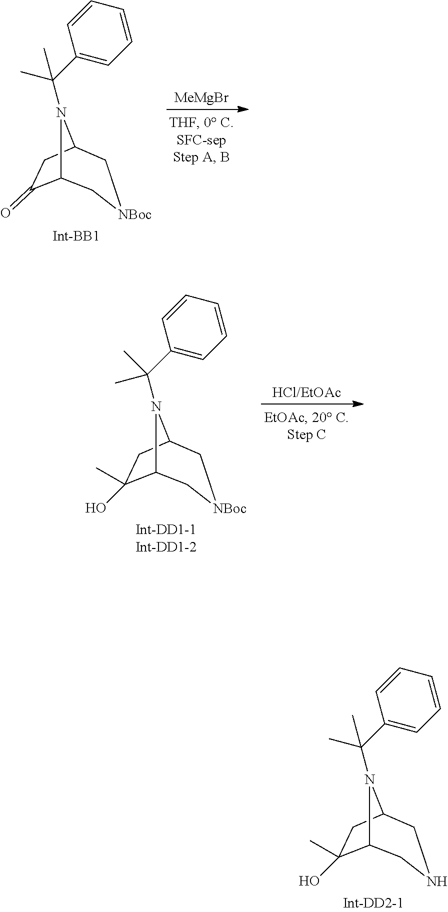 [SMILES: "MeMgBr" ; CC(C)(C)OC(=O)N1CC2CC(=O)C(C1)N2C(C)(C)c1ccccc1 ; CC(C)(C)OC(=O)N1CC2CC(C)(O)C(C1)N2C(C)(C)c1ccccc1 ; "HCl/EtOAc" ; CC1(O)CC2CNCC1N2C(C)(C)c1ccccc1]

### Step A: tert-butyl 6-hydroxy-6-methyl-8-(2-phenylpropan-2-yl)-3,8-diazabicyclo[3.2.1]octane-3-carboxylate (Int-DD1)

To a solution of tert-butyl 6-oxo-8-(2-phenylpropan-2-yl)-3,8-diazabicyclo[3.2.1]octane-3-carboxylate (Int-BB1) (0.8 g, 2.32 mmol) in THF (10 mL) was added methylmagnesium bromide (2,32 mL, 6.97 mmol, 3 M in THF) at 25° C. and stirred at 25° C. for 0.5 h. The reaction mixture was quenched with saturated aqueous NH₄Cl (5 mL) then extracted with EtOAc (5 mL×2). The combined organic phase was dried over Na₂SO₄, filtered and concentrated in vacuo. The residue was purified by flash silica gel chromatography (Eluent of 0 to 25% EtOAc/Pet, ether gradient) to give tert-butyl 6-hydroxy-6-methyl-8-(2-phenylpropan-2-yl)-3,8-diazabicyclo[3.2.1]octane-3-carboxylate (Int-DD1). MS (ESI): m/z (M+H)⁺ 361.

### Step B: tert-butyl 6-hydroxy-6-methyl-8-(2-phenylpropan-2-yl)-3,8-diazabicyclo[3.2.1]octane-3-carboxylate (Int DD1-1 and Int-DD1-2)

The racemic tert-butyl 6-hydroxy-6-methyl-8-(2-phenylpropan-2-yl)-3,8-diazabicyclo[3.2.1]octane-3-carboxylate (Int-DD1) (700 mg, 1.748 mmol) was separated by preparative SFC (Column D; Condition: 0.1% NH₄OH/IPA) to give tert-butyl 6-hydroxy-6-methyl-8-(2-phenylpropan-2-yl)-3,8-diazabicyclo[3.2.1]octane-3-carboxylate (Int-DD1-1, the first eluting). MS (ESI): m/z (M+H)⁺ 361. And tert-butyl 6-hydroxy-6-methyl-8-(2-phenylpropan-2-yl)-3,8-diazabicyclo[3.2.1]octane-3-carboxylate (Int-DD1-2, the second eluting isomer). MS (ESI): m/z (M+H)⁺ 361.

### Step C: 6-methyl-8-(2-phenylpropan-2-yl)-3,8-diazabicyclo[3.2.1]octan-6-ol (Int-DD2-1)

To a solution of tert-butyl 6-hydroxy-6-methyl-8-(2-phenylpropan-2-yl)-3,8-diazabicyclo[3.2.1]octane-3-carboxylate (Int-DD1-1) (50 mg, 0.139 mmol) in EtOAc (2 mL) was added HCl/EtOAc (2 mL, 8.0 mmol, 4N) at 20° C. and stirred at 20° C. for 12 h. The mixture was concentrated in vacuo to afford crude 6-methyl-8-(2-phenylpropan-2-yl)-3,8-diazabicyclo[3.2.1]octan-6-ol (Int-DD2-1). MS (ESI): m/z (M+H)⁺ 261.

### Intermediate EE2: 6-fluoro-8-(2-phenylpropan-2-yl)-3,8-diazabicyclo[3.2.1]octane

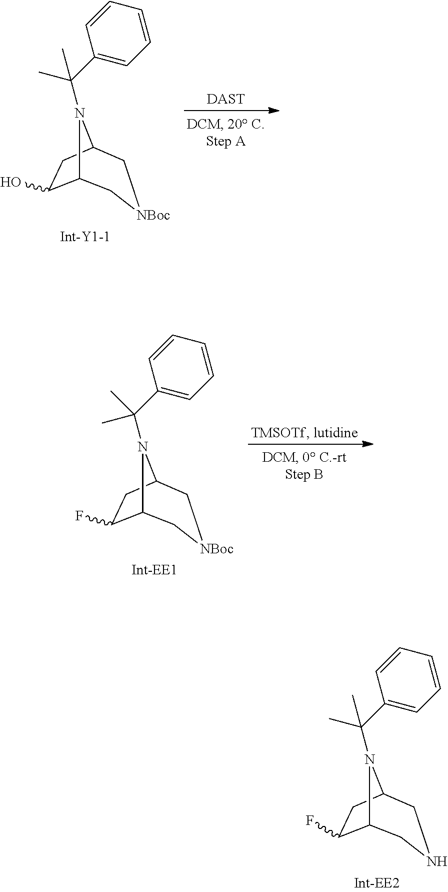 [SMILES: CC(C)(C)OC(=O)N1CC2CC(O)C(C1)N2C(C)(C)c1ccccc1 ; "DAST" ; CC(C)(C)OC(=O)N1CC2CC(F)C(C1)N2C(C)(C)c1ccccc1 ; "TMSOTf, lutidine" ; CC(C)(c1ccccc1)N1C2CNCC1C(F)C2]

### Step A: tert-butyl 6-fluoro-8-(2-phenylpropan-2-yl)-3,8-diazabicyclo[3.2.1]octane-3-carboxylate (Int-EE1)

To a solution of tert-butyl 6-hydroxy-8-(2-phenylpropan-2-yl)-3,8-diazabicyclo[3.2.1]octane-3-carboxylate (Int-Y1-1) (200 mg, 0.577 mmol) in DCM (2 mL) was added DAST (0.114 mL, 0.866 mmol) at 0° C. under N, atmosphere. The mixture was stirred at 0° C. for 2 h. The mixture was diluted with NaHCO₃ solution (5 mL), extracted with DCM (3×5 mL), dried over Na₂SO₄, filtered and the solvent was evaporated under reduced pressure to give the crude product. The crude product was purified by flash silica gel chromatography (eluent of 0 to 30% EtOAc/Pet, ether gradient) to give tert-butyl 6-fluoro-8-(2-phenylpropan-2-yl)-3,8-diazabicyclo[3.2.1]octane-3-carboxylate (Int-EE1) with the F group in an equatorial position. MS (ESI): m/z (M+H)⁺ 349. Step B: 6-fluoro-8-(2-phenylpropan-2-yl)-3,8-diazabicyclo[3.2.1]octane (Int-EE2)

To a solution of tert-butyl 6-fluoro-8-(2-phenylpropan-2-yl)-3,8-diazabicyclo[3.2.1]octane-3-carboxylate (Int-EE1) (170 mg, 0.488 mmol) in DCM (2 mL) was added trimethylsilyl trifluoromethanesulfonate (434 mg, 1.951 mmol) and 2,6-dimethylpyridine (314 mg, 2.93 mmol) at 0° C. under N2 atmosphere. The mixture was stirred at 0° C. for 1 h. The mixture was evaporated under reduced pressure to give the crude product 6-fluoro-8-(2-phenylpropan-2-yl)-3,8-diazabicyclo[3.2.1]octane (Int-EE2). MS (ESI): m/z (M+H)⁺ 249.

### Intermediate FF3-1 and FF3-2: 6-fluoro-8-(2-phenylpropan-2-yl)-3,8-diazabicyclo[3.2.1]octane (Int-FF3-1 and Int FF3-2)

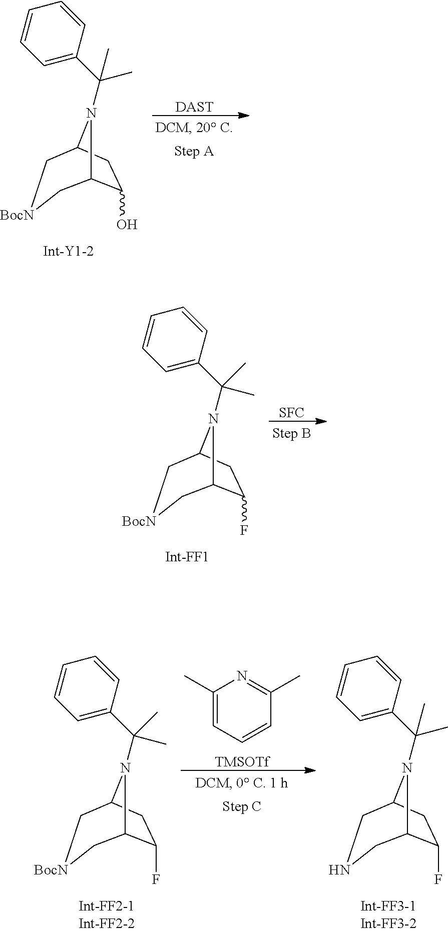

### Step A: tert-butyl 6-fluoro-8-(2-phenylpropan-2-yl)-3,8-diazabicyclo[3.2.1]octane-3-carboxylate (Int-FF1)

To a solution of tert-butyl 6-hydroxy-8-(2-phenylpropan-2-yl)-3,8-diazabicyclo[3.2.1]octane-3-carboxylate (Int-Y1-2) (1.9 g, 5.48 mmol) in DCM (25 mL) was added diethylaminosulfur trifluoride (3.54 g, 21.9 mmol) at 0° C. under N₂ atmosphere. The mixture was stirred at 0° C. for 2 h. The mixture was cooled, diluted with saturated aqueous NaHCO₃(15 mL), extracted with DCM (2×20 mL), dried over Na₂SO₄, filtered and the solvent was evaporated under reduced pressure to give the crude product. The crude product was purified by flash silica gel chromatography (Eluent Of 0-30% EtOAc/Pet, ether gradient) to give tert-butyl 6-fluoro-8-(2-phenylpropan-2-yl)-3,8-diazabicyclo[3.2.1]octane-3-carboxylate (Int-FF1). MS (EST) [M+H]⁺ m/z: 349.

### Step B: tert-butyl 6-fluoro-8-(2-phenylpropan-2-yl)-3,8-iazabicyclo[3.2.1]octane-3-carboxylate (Int-FF2)

tert-Butyl 6-fluoro-8-(2-phenylpropan-2-yl)-3,8-diazabicyclo[3.2.1]octane-3-carboxylate (Int-FF1) (900 mg, 2.58 mmol) was separated by SFC (Column 0; Condition 0.1% NH₄OH in EtOH) to give tert-butyl 6-fluoro-8-(2-phenylpropan-2-yl)-3,8-diazabicyclo[3.2.1]octane-3-carboxylate (Int-FF2-1) as the first eluting isomer. MS (ESI) [M+H]⁺ m/z: 349. And tert-butyl 6-fluoro-8-(2-phenylpropan-2-yl)-3,8-diazabicyclo[3.2.1]octane-3-carboxylate (Int-FF2-2) as the second eluting isomer. MS (ESI) [M+H]⁺ m/z: 349.

### Step C: 6-fluoro-8-(2-phenylpropan-2-yl)-3,8-diazabicyclo[3.2.1]octane (Int-FF3-1)

To a solution of tert-butyl 6-fluoro-8-(2-phenylpropan-2-yl)-3,8-diazabicyclo[3.2.1]octane-3-carboxylate (300 mg, 0.861 mmol) (Int-FF2-1) in DCM (2 mL) was added trimethylsilyl trifluoromethanesulfonate (765 mg. 3.44 mmol) and 2,6-dimethylpyridine (554 mg, 5.17 mmol) at 0° C. under N₂ atmosphere. The mixture was stirred at 0° C. for 1 h. The mixture was evaporated under reduced pressure to give the crude product 6-fluoro-8-(2-phenylpropan-2-yl)-3,8-diazabicyclo[3.2.1]octane (Int-FF3-1). MS (ESI) [M+H]⁺ nm z: 249.

Int-FF3-2 was prepared in the same way from Int-FF2-2. MS (ESI) [M+H]⁺ m/z: 249.

### Intermediate GG2: 9-(2-phenylpropan-2-yl)-7.9-diazatricyclo[3.3,1]nonane (Int-GG2)

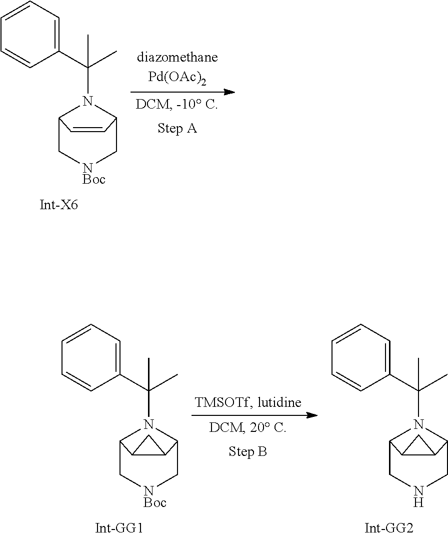 [SMILES: CC(C)(C)OC(=O)N1CC2C=CC(C1)N2C(C)(C)c1ccccc1 ; CC(C)(C)OC(=O)N1CC2C3CC3C(C1)N2C(C)(C)c1ccccc1 ; CC(C)(c1ccccc1)N1C2CNCC1C1CC12]

### Step A: tert-butyl 9-(2-phenylpropan-2-yl-7.9-diazatricyclo[3.3,1,0^{2,4}]nonane-7-carboxylate (Int-GG1)

To a solution of tert-butyl (1R,5S)-8-(2-phenylpropan-2-yl)-3,8-diazabicyclo[3.2.1]oct-6-ene-3-carboxylate (Int-X6) (300 mg, 0.913 mmol) and palladium (11) acetate (10.3 mg, 0.046 mmol) in DCM (5.0 mL) was added diazomethane (30.4 mL. 9.13 mmol) (˜0.3 M in Et₂O) at -20 ˜−10° C. The reaction mixture was stirred at -20 to −10° C. for 2 h. The mixture was filtered and the filtered cake was washed with DCM (10 mL). The filtrate was concentrated in vacuo to give crude tert-butyl 9-(2-phenylpropan-2-yl)-7,9-diazatricyclo[3.3,1,0^{2,4}]nonane-7-carboxylate (Int-GG1). MS (EST): m/z (M+H)⁺ 343.

### Step B: 9-(2-phenylpropan-2-yl)-7.9-diazatricyclo[3.3,1,02,4]nonane (Int-GG2)

To a solution of tert-butyl 9-(2-phenylpropan-2-yl)-7,9-diazatricyclo[3.3,1,0^{2,4}]nonane-7-carboxylate (Int-GG1) (100 mg, 0.292 mmol) in DCM (5 mL) was added 2,6-dimethylpyridine (188 mg, 1.75 mmol) and trimethylsilyl trifluoromethanesulfonate (260 mg, 1.17 mmol) at 20° C. The resulting mixture was stirred at 20° C. for 2 min. The reaction mixture was quenched with aqueous sodium hydrogen carbonate (3.0 mL) and extracted with DCM (2×10 mL). The combined organic layers were washed with brine (8 mL), dried over Na₂SO₄, filtered and the solvent was evaporated under reduced pressure to give 9-(2-phenylpropan-2-yl)-7,9-diazatricyclo[3.3,1,0,^{2,4}]nonane (Int-GG2). MS (ESI): m/z (M+H)⁺ 243.

### Intermediate HH4: tert-butyl 1.5-dimethyl-3,8-diazabicyclo[3.2.1]octane-8-carboxylate (Int-HH4)

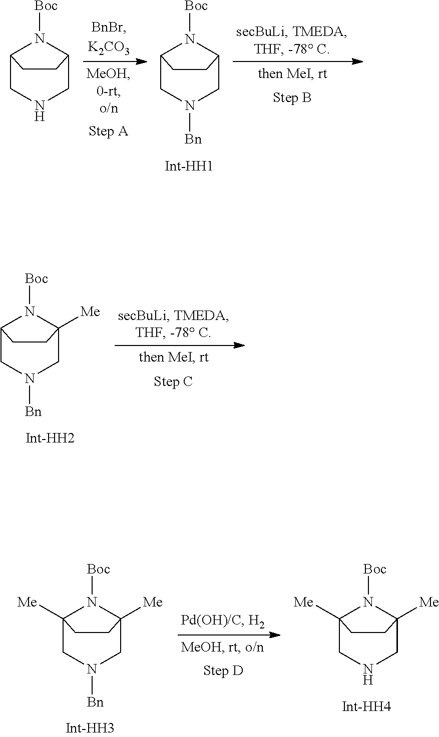 [SMILES: CC(C)(C)OC(=O)N1C2CCC1CNC2 ; CC(C)(C)OC(=O)N1C2CCC1CN(Cc1ccccc1)C2 ; CC(C)(C)OC(=O)N1C2CCC1(C)CN(Cc1ccccc1)C2 ; CC(C)(C)OC(=O)N1C2(C)CCC1(C)CN(Cc1ccccc1)C2 ; CC(C)(C)OC(=O)N1C2(C)CCC1(C)CNC2]

### Step A: tert-butyl 3-benzyl-3,8-diazabicyclo[3.2.1]octane-8-carboxylate (Int-HH2)

To a stirred solution of tert-butyl 3.8-diazabicyclo[3.2.1]octane-8-carboxylate (250 g, 1.18 mol) and MeOH (5 L) was added K₂CO₃ (407 g, 2.94 mol) in portions at room temperature under nitrogen atmosphere. The reaction mixture was cooled down to O ° C. followed by the addition of benzyl bromide (242 g, 1.41 mol) dropwise at 0° C. The resulting mixture was stirred overnight at room temperature. The reaction was quenched with water/ice (5 L). The resulting mixture was extracted with EtOAc (3×3 L). The combined organic layers were dried over anhydrous Na₂SO₄. After filtration, the filtrate was concentrated under reduced pressure. The residue was purified by silica gel column chromatography, eluted with Pet.ether/EtOAc (1:0-10:1) to afford tert-butyl 3-benzyl-3,8-diazabicyclo[3.2.1]octane-8-carboxylate (Int-HH1). MS (ESI): m/z 303 [M+H]⁺.

### Step B: tert-butyl 3-benzyl-1-methyl-3,8-diazabicyclo[3.2.1]octane-8-carboxylate (Int-HH2)

Into a 10 L 4-necked round-bottom flask were added tert-butyl 3-benzyl-3,8-diazabicyclo[3.2.1]octane-8-carboxylate (Int-HH1) (248 g, 820 mmol) and THF (5 L) at room temperature. The reaction mixture was cooled down to −78° C. To the stirred mixture were added TMEDA (110 g, 943 mmol) and a 1.3 M solution of sec-BuLi in hexanes (947 mL, 1.23 mol) dropwise at −78° C. under nitrogen atmosphere, and the mixture was stirred for additional 2 h at −78° C. To the above mixture was added Mel (175 g, 1.23 mol) dropwise at −78° C. The resulting mixture was stirred for an additional 1 h at room temperature. The reaction was quenched with sat. NH₄Cl (2 L) at 0° C. The resulting mixture was extracted with EtOAc (2×2L). The combined organic layers were dried over anhydrous Na₂SO₄. After filtration, the filtrate was concentrated under reduced pressure. The residue was purified by silica gel column chromatography, eluted with Pet.ether/EtOAc (1:0-10:1) to afford tert-butyl 3-benzyl-1-methyl-3,8-diazabicyclo[3.2.1]octane-8-carboxylate (Int-HH2). MS (ESI): m/z 317[M+H]⁺.

### Step C: tert-butyl 3-benzyl-1,5-dimethyl-3,8-diazabicyclo[3.2.1]octane-8-carboxylate (Int-HH3)

Into a 10 L 4-necked round-bottom flask were added tert-butyl 3-benzyl-1-methyl-3,8-diazabicyclo[3.2.1]octane-8-carboxylate (Int-HH2) (218 g, 689 mmol) and THF (4 L) at room temperature. The reaction mixture was cooled down to −78° C. To the stirred mixture were added TMEDA (92.1 g, 792 mmol) and a 1.3 M solution of sec-BuLi in hexanes (795 mL, 1.03 mol) dropwise at -78° C. under nitrogen atmosphere. The reaction mixture was stirred for additional 2 h at −78° C. To the above mixture was added Mel (147 g, 1.03 mol) dropwise at −78° C. The resulting mixture was stirred for an additional 1 h at room temperature. The reaction was quenched with sat. NH₄Cl (2 L) at 0° C. The resulting mixture was extracted with EtOAc (2×2000 mL). The combined organic layers were dried over anhydrous Na₂SO₄. After filtration, the filtrate was concentrated under reduced pressure. The residue was purified by silica gel column chromatography, eluted with Pet.ether/EtOAc (1:0-10:1) to afford tert-butyl 3-benzyl-1,5-dimethyl-3,8-diazabicyclo[3.2.1]octane-8-carboxylate (Int-HH3). MS (ESI): m/z 331 [M+H]⁺.

### Step D: tert-butyl 1.5-dimethyl-3,8-diazabicyclo[3.2.1]octane-8-carboxylate (Int-HH4)

To a stirred solution of tert-butyl 3-benzyl-1,5-dimethyl-3,8-diazabicyclo[3.2.1]octane-8-carboxylate (Int-HH3) (82.0 g, 248 mmol) and MeOH (2 L) was added Pd(OH)₂/C (40.0 g, 10% w/w) in portions at room temperature under hydrogen atmosphere. The resulting mixture was stirred overnight at room temperature. The resulting mixture was filtered and the filtrate was concentrated under reduced pressure to yield tert-butyl 1.5-dimethyl-3,8-diazabicyclo[3.2.1]octane-8-carboxylate (Int-HH4). MS (ESI): m/z 241 [M+H]⁺.

### Intermediate II1-1 and Intermediate II1-2: tert-butyl (1R,5S)-1-methyl-3,8-diazabicyclo[3.2.1]octane-8-carboxylate (Int-II1-1) and tert-butyl (1S,5R)-1-methyl-3,8-diazabicyclo[3.2.1]octane-8-carboxylate (Int-II1-2)

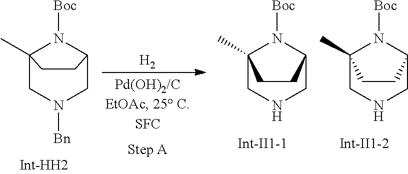

To a solution of tert-butyl 3-benzyl-1-methyl-3,8-diazabicyclo[3.2.1]octane-8-carboxylate (Int-HH2) (52.0 g, 164 mmol) in EtOAc (1 L) was added Pd(OH)₂/C (23.1 g, 16.4 mmol) under N₂. The reaction mixture was degassed under vacuum and purged with H₂ several times. The resulting mixture was stirred under Hz (15 psi) at 25° C. for 12 h. The product mixture was filtered and the filtrate was concentrated. The residue obtained was resolved by SFC (Column A; Condition; 25% 0.1% NH₄OH in MeOH). The first eluting peak corresponds to tert-butyl (1R5S)-1-methyl-3,8-diazabicyclo[3.2.1]octane-8-carboxylate (Int-II1-1). ¹H NMR (400 MHz, Methanol-d₆) δ 4.15 (br d, J=7.2 Hz, 1H), 2.86 (br t, J=11.6 Hz, 2H), 2.48-2.60 (m, 2H), 1.90-2.05 (m, 2H), 1.69-1.80 (m, 2H), 1.37-1.54 (m, 12H). The second eluting peak corresponds to tert-butyl (1S,5R)-1-methyl-3,8-diazabicyclo[3.2.1]octane-8-carboxylate (Int-II1-2). ¹H NMR (400 MHz, Methanol-d₄) δ 4.15 (br d, J=7.2 Hz. 1H), 2.86 (br t, J=11.6 Hz, 2H), 2.46-2.60 (m, 2H). 1.90-2.07 (m, 2H), 1.69-1.80 (m, 2H). 1,37-1.55 (m, 12H). The absolute stereochemistry was determined by small molecule x-ray crystallography of a further elaborated molecule.

### Intermediate JJ-2: tert-butyl 1-ethyl-3,8-diazabicyclo[3.2.1]octane-8-carboxylate (Int-JJ-2)

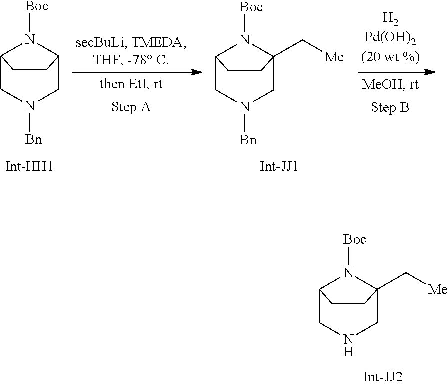 [SMILES: CC(C)(C)OC(=O)N1C2CCC1CN(Cc1ccccc1)C2 ; CCC12CCC(CN(Cc3ccccc3)C1)N2C(=O)OC(C)(C)C ; CCC12CCC(CNC1)N2C(=O)OC(C)(C)C]

### Step A; tert-butyl 3-benzyl-1-ethyl-3,8-diazabicyclo[3.2.1]octane-8-carboxylate (Int-JJ-1)

tert-Butyl 3-benzyl-3,8-diazabicyclo[3.2.1]octane-8-carboxylate (Int-HH1) (0.5 g, 1.653 mmol) was added to a round bottomed flask, which was evacuated and back-filled with nitrogen three times. THF (10 mL) was added and the reaction was cooled to −78° C. TMEDA (0.279 mL, 1.852 mmol) was added, followed by sec-butylthium (1.77 mL, 2.48 mmol, 1.4M in hexanes). The mixture was stirred for 1 h and then iodoethane (0.256 mL, 2.480 mmol) was added. The reaction mixture was allowed to warm slowly to room temperature. The reaction was concentrated and the residual material was purified by silica gel column chromatography (eluent: 0-100% ethyl acetate in hexanes) to yield tert-butyl 3-benzyl-1-ethyl-3,8-diazabicyclo[3.2.1]octane-8-carboxylate (Int-JJ-1). MS (ESI): m/z 331 [M+H]⁺.

### Step B: tert-butyl 1-ethyl-3,8-diazabicyclo[3.2.1]octane-8-carboxylate (Int-JJ-2)

tert-Butyl 3-benzyl-1-ethyl-3,8-diazabicyclo[3.2.1]octane-8-carboxylate (Int-JJ-1) (327 mg, 0.99 mmol) was added to a round bottomed flask and dissolved in the MeOH (6.5 mL). To the solution was added Pd(OH)₂/C (20 wt %) (69.5 mg). The mixture was capped with a septum and the head space purged with hydrogen then left under a balloon of hydrogen (1 atm) at room temperature overnight. The solution was filtered and concentrated to afford tert-butyl 1-ethyl-3,8-diazabicyclo[3.2.1]octane-8-carboxylate (Int-JJ-2). MS (ESI): m/z 241 [M+H]⁺. ¹H NMR (600 MHz, Methanol-d₆) δ 4.17 (d, J=6.6 Hz, 1H), 2.96 (d, J=12.8 Hz, 1H), 2.86 (d, J=12.8 Hz. 1H). 2.64 (m, 1H), 2.60 (d, J=12.8 Hz, 1H). 2.07-1.83 (multiple peaks, 3H), 1.78 (m, 1H), 1.63 (m, 1H), 1.51 (s, 9H), 1.50 (m, overlapping peaks, 2H), 0.94 (t, J=7.4 Hz, 3H).

### Intermediate KK2: tert-butyl 1-fluoro-3,8-diazabicyclo[3.2.1]octane-8-carboxylate (Int-KK2)

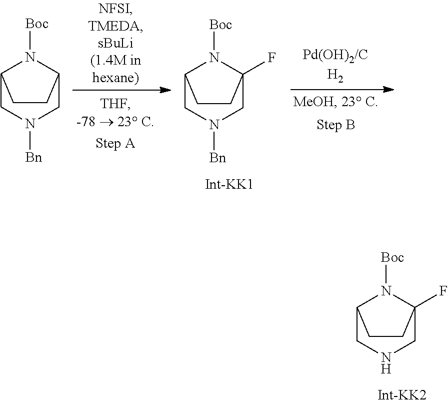 [SMILES: CC(C)(C)OC(=O)N1C2CCC1CN(Cc1ccccc1)C2 ; CC(C)(C)OC(=O)N1C2CCC1(F)CN(Cc1ccccc1)C2 ; CC(C)(C)OC(=O)N1C2CCC1(F)CNC2]

### Step A: tert-butyl 3-benzyl-1-fluoro-3,8-diazabicyclo[3.2.1]octane-8-carboxylate (Int-KK1)

tert-Butyl 3-benzyl-3,8-diazabicyclo[3.2.1]octane-8-carboxylate (Int-HH1)(1g, 3.31 mmol) was added to a round bottomed flask. The flask was evacuated and back filled with nitrogen three times, followed by the addition of THF (20 mL). TMEDA (0.559 mL, 3.70 mmol) was added and the reaction was cooled to −78° C. sBuLi (3.54 mL, 4.96 mmol, 1.4 M in hexanes) was added dropwise, keeping the internal temp below −60° C. The solution was stirred at -78° C. for 30 mins. To this solution was added N-fluorobenzenesulfonimide (2.09 g, 6.61 mmol) as a solution in THF (20 mL). The mixture was stirred for 30 min at -78° C. and allowed to slowly warm to room temperature. Water (20 mL) was added to the solution and the organic layer was extracted with EtOAc, dried over MgSO₄, then filtered and concentrated to dryness on a rotavap under reduced pressure. The crude mixture was purified via silica gel chromatography (Eluent 0-100% EtOAc in Hexanes) to yield tert-butyl 3-benzyl-1-fluoro-3,8-diazabicyclo[3.2.1]octane-8-carboxylate (Int-KK1). ¹H NMR (600 MHz, Acetonitrile-d3) δ 7.35 (s, 4H), 7.29 (h, J=4.0 Hz, 1H), 4.24-4.14 (m, 1H), 3.60 (d, J=3.0 Hz. 2H), 2.84 (dd, J=9.7, 2.7 Hz, 1H), 2.66 (d, J=9.7 Hz, 1H), 2.52 (ddd, J=11.3, 4.2, 2,3 Hz, 1H), 2.41-2.30 (m, 1H), 2.32-2.23 (m, 1H), 2.01-1.85 (m, 2H), 1.64-1.52 (m, 1H), 1.45 (s, 9H).

### Step B: tert-butyl 1-fluoro-3,8-diazabicyclo[3.2.1]octane-8-carboxylate (Int-KK2)

tert-Butyl 3-benzyl-1-fluoro-3,8-diazabicyclo[3.2.1]octane-8-carboxylate (Int-KK1) (0.358 g, 1.12 mmol) was added to a flask and dissolved in MeOH (7.16 mL). To the solution was added palladium hydroxide on carbon (0.078 g, 0.112 mmol). The mixture was subjected to balloon hydrogenation and allowed to stir at room temperature overnight. The reaction mixture was filtered over a bed of CELITE and MgSO₄. The filtrate was concentrated to dryness to yield tert-butyl 1-fluoro-3,8-diazabicyclo[3.2.1]octane-8-carboxylate (Int-KK2) which was used in the next step without further purification. ¹H NMR (600 MHz, Acetonitrile-d3) δ 4.19 (d, J=6.6 Hz. 1H), 3.19 (d, J=11.7 Hz, 1H), 2.81 (dd, J=12,3, 5.0 Hz, 2H), 2.47 (dd, J=12.5, 5.3 Hz, 1H), 2.38-2.10 (m, 3H), 2.09-1.89 (m, 2H).

### Intermediate LL2: tert-Butyl (1S,4S)-1-methyl-2,5-diazabicyclo[2.2.2]octane-2-carboxylate (Int-LL2)

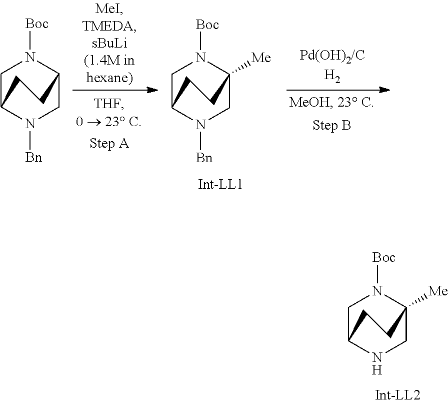 [SMILES: CC(C)(C)OC(=O)N1C[C@@H]2CC[C@H]1CN2Cc1ccccc1 ; CC(C)(C)OC(=O)N1C[C@@H]2CC[C@@]1(C)CN2Cc1ccccc1 ; CC(C)(C)OC(=O)N1C[C@@H]2CC[C@@]1(C)CN2]

### Step A: tert-Butyl (1S,4S)-5-benzyl-1-methyl-2,5-diazabicyclo[2.2.2]octane-2-carboxylate (Int-LL1)

tert-Butyl (1S,4S)-5-benzyl-2,5-diazabicyclo[2.2.2]octane-2-carboxylate (2.00 g, 6.61 mmol) was added to a round bottomed flask. The flask was evacuated and back filled with nitrogen three times. THF (40 mL) was added and the reaction was cooled to 0° C. TMEDA (1.118 mL, 7.41 mmol) was added, followed by s-BuLi (7.09 mL, 9.92 mmol. 1.4 M in hexanes). The mixture was stirred for 20 min at 0° C. and then methyl iodide (0.620 mL, 9.92 mmol) was added and the reaction was stirred at 0° C. for 30 min. After 30 min, the mixture was allowed to slowly warm to room temperature and quenched with saturated NH₄Cl (10 mL). The reaction was diluted with EtOAc (50 mL) and washed with brine solution (100 mL). The organic layer was separated, dried over MgSO₄, and concentrated. The desired compound was purified by column chromatography on silica gel with a 0-100% EtOAc/Hex gradient to give tert-butyl (1S,4S)-5-benzyl-1-methyl-2,5-diazabicyclo[2.2.2]octane-2-carboxylate (Int-LL1). MS (ESI): m/z (M+H)⁺ 317.

### Step B: tert-butyl (1S,4S)-1-methyl-2,5-diazabicyclo[2.2.2]octane-2-carboxylate (Int-LL2)

tert-butyl (1S,4S)-5-benzyl-1-methyl-2,5-diazabicyclo[2.2.2]octane-2-carboxylate (Int-LL) (500 mg, 1.58 mmol) was added to a round bottomed flask and dissolved in the MeOH (10 mL). To the solution was added Pd(OH)₂/C (20 wt %) (II1.0 mg). The mixture was capped with a septum and the head space purged with hydrogen then left under a balloon of hydrogen (1 atm) at room temperature for 3 h. The solution was filtered and concentrated to afford tert-butyl (1S,4S)-1-methyl-2,5-diazabicyclo[2.2.2]octane-2-carboxylate (Int-LL2). MS (ESI): m/z (M+H)⁺ 227.

### Intermediate MM2: tert-butyl (1S,3R4S)-3-methyl-2,5-diazabicyclo[2.2.2]octane-2-carboxylate (Int-MM2)

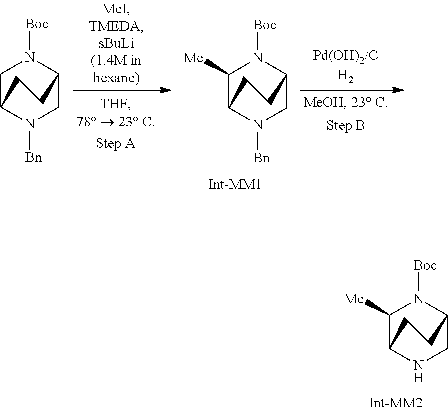 [SMILES: CC(C)(C)OC(=O)N1C[C@@H]2CC[C@H]1CN2Cc1ccccc1 ; C[C@@H]1[C@@H]2CC[C@@H](CN2Cc2ccccc2)N1C(=O)OC(C)(C)C ; C[C@@H]1[C@@H]2CC[C@@H](CN2)N1C(=O)OC(C)(C)C]

### Step A: tert-butyl (1S,3R,4S)-5-benzyl-3-methyl-2,5-diazabicyclo[2.2.2]octane-2-carboxylate (Int-MM1)

tert-Butyl (1S,4S)-5-benzyl-2,5-diazabicyclo[2,2,2]octane-2-carboxylate (1 g, 3.31 mmol) was added to a round bottomed flask, and the flask was evacuated and back filled with nitrogen three times. THF (20 mL) and TMEDA (0.559 mL, 3.70 mmol) was added and the reaction was cooled to −78° C. To this solution was added s-BuLi (3.54 mL, 4.96 mmol, 1.4 M in hexanes). The mixture was stirred at −78° C. for 60 min and then methyl iodide (0.310 mL, 4.96 mmol) was added. The mixture was stirred for another 60 min at −78° C. then warmed to room temperature. The reaction was quenched with saturated NH₄CI (10 mL). The reaction was diluted with EtOAc (50 mL) and washed with brine (100 mL). The organic layer was separated, dried over MgSO₄, and concentrated. The crude mixture was purified by column chromatography on silica gel with a 0-100% EtOAc/Hex gradient to yield tert-butyl (1S,3R,4S)-5-benzyl-3-methyl-2,5-diazabicyclo[2.2.2]octane-2-carboxylate (Int-MM1). MS (ESI): m/z (M+H)⁺ 317.

### Step B: tert-butyl (1S,3R,4S)-3-methyl-2,5-diazabicyclo[2.2.2]octane-2-carboxylate (Int-MM2)

tert-butyl (1S,3R,4S)-5-benzyl-3-methyl-2,5-diazabicyclo[2.2.2]octane-2-carboxylate (Int-MM1) (500 mg, 1.58 mmol) was added to a round bottomed flask and dissolved in MeOH (10 mL). To the solution was added Pd(OH)₂/C (20 wt %6) (II1 mg). The mixture was capped with a septum and the head space purged with hydrogen then left under a balloon of hydrogen (1 atm) at room temperature for 6 h. The solution was filtered and concentrated to afford tert-butyl (1S,3R,4S)-3-methyl-2,5-diazabicyclo[2.2.2]octane-2-carboxylate (Int-MM2). MS (ESI): m/z (M+H)⁺ 227.

### Intermediate N,N1: tert-butyl (1S,4S)-6-vinyl-2,5-diazabicyclo[2.2.2]octane-2-carboxylate (Int-N,N1)

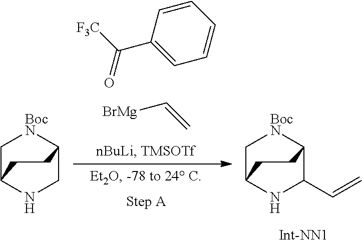

To a solution of tert-butyl (1S,4S)-2,5-diazabicyclo[2.2.2]octane-2-carboxylate (4.25 g, 20 mmol) in anhydrous ether (22.5 mL) cooled to −78° C. was slowly added a 2.5 M solution of n-BuLi in hexanes (8.00 mL, 20 mmol) under nitrogen. The resulting solution was stirred at the same temperature for 10 min. To this was then added via cannula a solution of 2,2,2-trifluoroacetophenone (4.18 g, 24.0 mmol) in anhydrous ether (20 mL). The resulting mixture was stirred at −78° C. for 10 min, followed by the addition of a 1 M solution of vinylmagnesium bromide in THF (30.0 mL, 30.0 mmol) in one portion, followed immediately by the addition of TMSOTf (4.34 mL, 24.0 mmol). The reaction mixture was stirred at room temperature for 2 h. The reaction mixture was then cooled to 0° C. and quenched via the addition of methanol (10 mL). The resulting mixture was diluted with ether (10 mL) and washed with 1 M NaOH solution (10 mL). The aqueous layer was then extracted with ether (3×10 mL) and the combined organic layers washed with brine (10 mL) and dried over anhydrous Na₂SO₄. Solvent was then removed under reduced pressure and the residue purified by silica gel chromatography (0-10% MeOH-DCM) to afford terl-butyl (1S,4S)-6-vinyl-2,5-diazabicyclo[2.2.2]octane-2-carboxylate (Int-N,N1) as a 2.5:1 mixture of trans:cis diastereomers. MS (ESI): m/z 239 [M+H]⁺.

Int-NO1 in the table below was synthesized via the same synthetic route using Int-N,N1 and methyl magnesium bromide (3M in diethyl ether) as starting materials.

| | | | [M + H]⁺ |
|---|---|---|---|
| Int | Structure | Compound Name | Found |
| Int-NO-1 | 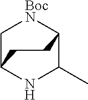 [SMILES: CC1N[C@H]2CC[C@@H]1N(C(=O)OC(C)(C)C)C2] | tert-butyl (1S,4S)-6- methyl-2,5- diazabicyclo[2.2.2] octane-2-carboxylate | 227 |
| | (1:1 mixture of | | |
| | diastereomers | | |

### Intermediate O02-1: (1S,3R,4S)-2-(3.4-dimethoxybenzyl)-3-vinyl-2,5-diazabicyclo[2.2.2]octane (Int-QQ2-1)

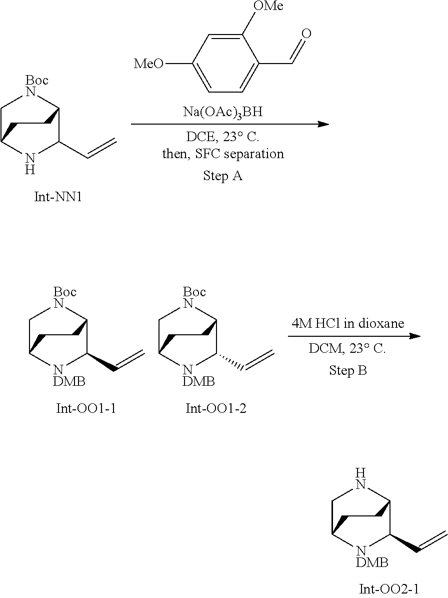

### Step A: tert-butyl (1S,4S,6R)-5-(2.4-dimethoxybenzyl)-6-vinyl-2,5-diazabicyclo[2,2,2]octane-2-carboxylate (Int-OO1-1)

To a vial containing tert-butyl (1S,4S)-6-vinyl-2,5-diazabicyclo[2.2.2]octane-2-carboxylate (Int-N,N1) (1147 mg, 4.81 mmol) and 2,4-dimethoxybenzaldehyde (960 mg, 5.78 mmol) in DCE (12.00 mL) was added sodium triacetoxyborohydride (1632 mg, 7.70 mmol). The reaction mixture was stirred at room temperature overnight. The reaction was quenched with 1 M NaOH and the organic layer was collected by use of a phase separator. The crude was purified by silica gel column chromatography, eluting from 0 to 25% ethyl acetate in hexanes to yield the desired product as a mixture of cis and trans isomers. The mixture was subjected to chiral SFC separation (Column F: 15% MeOH w/0.1% NH₄OH) to give tert-butyl (1S,4S,6R)-5-(2,4-dimethoxybenzyl)-6-vinyl-2,5-diazabicyclo[22.2]octane-2-carboxylate (Int-QQ1-1, Peak 1) as the syn isomer. MS (ESI): m/z 389 [M+H]⁺.

### Step B: (1S,3R,4S)-2-(2.4-dimethoxybenzyl)-3-vinyl-2,5-diazabicyclo[2.2.2]octane (Int-QQ2-1)

To a vial of tert-butyl (1S,4S,6R)-5-(2,4-dimethoxybenzyl)-6-vinyl-2,5-diazabicyclo[2.2.2]octane-2-carboxylate (Int-QQ1-1) (215 mg, 0.553 mmol) in DCM (2767 μL) was added 4M HCl in dioxane (692 μL, 2.77 mmol) at room temperature and the mixture was stirred at for 2 h. Diethyl ether was added and the heterogeneous mixture was filtered to collect the solid. The solid was dried under high vacuum to give (1S,3R,4S)-2-(2,4-dimethoxybenzyl)-3-vinyl-2,5-diazabicyclo[2.2.2]octane (Int-QQ2-1). MS (ESI): m/z 289 [M+H]⁺.

### Intermediate O02-2: (1S,3S,4S)-2-(3.4-dimethoxybenzyl)-3-vinyl-2,5-diazabicyclo[2.2.2]octane (Int-QQ2-2)

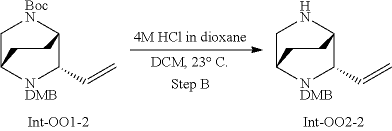

(1S,35.4S)-2-(3.4-dimethoxybenzyl)-3-vinyl-2,5-diazabicyclo[2.2.2]octane (Int-QQ2-2) was prepared via an analogous deprotection step as Int-QQ2-1 above.

### Intermediate PP4-1 and Intermediate PP4-2: tert-Butyl 1-(cyanomethyl)-3,8-diazabicyclo[3.2.1]octane-8-carboxylate (Int-PP4-1 and Int-PP4-2)

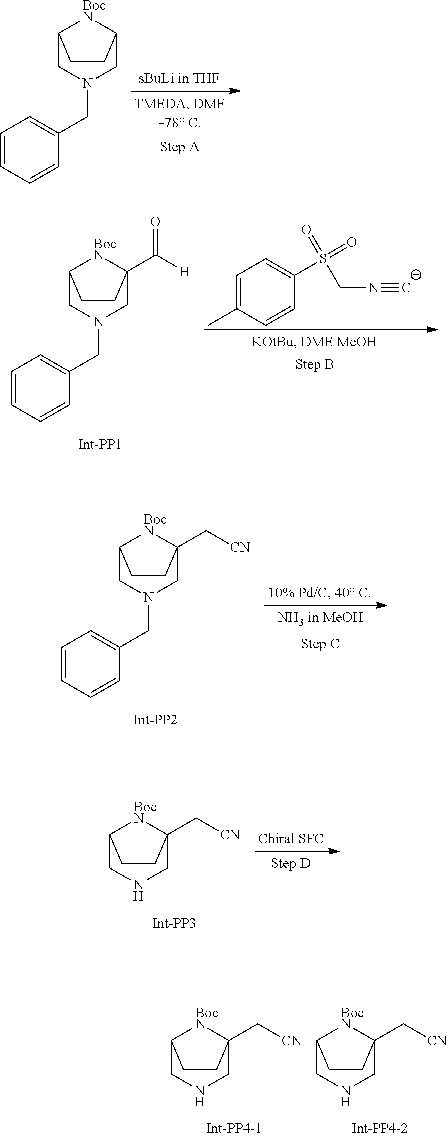

### Step A: tert-butyl 3-benzyl-1-formyl-3,8-diazabicyclo[3.2.1]octane-8-carboxylate (Int-PP1)

To a solution of tert-butyl 3-benzyl-3,8-diazabicyclo[3.2.1]octane-8-carboxylate (2 g, 6.61 mmol) in THF (6 mL) and TMEDA (1.12 mL, 7.41 mmol), cooled to 0° C. was added 1.4M s-BuLi in hexanes (7.09 mL, 9.92 mmol) and the reaction was stirred for 45 mins. To the above cooled reaction, DMF (0.61 mL, 7.94 mmol) was added slowly and warmed to room temperature over an hour. The reaction was quenched with aqueous saturated NH₄Cl solution. The reaction mixture was partitioned into ethyl acetate and aq. saturated NH₄C₁. The aqueous layer was extracted twice and the organic layer was separated, dried over MgSO₄, filtered and excess solvent was removed under reduced pressure. The crude mixture was purified on a silica gel column using 0-70% ethyl acetate in hexanes to give tert-butyl 3-benzyl-1-formyl-3,8-diazabicyclo[3.2.1]octane-8-carboxylate (Int-PPI). MS (ESI) m/z [M+H]⁺ 331.

### Step B: tert-butyl 3-benzyl-1-(cyanomethyl)-3,8-diazabicyclo[3.2.1]octane-8-carboxylate (Int-PP2)

Potassium tert-butoxide (340 mg, 3.03 mmol) was dissolved in DME (10 mL) and tosylmethyl isocyanide (325 mg, 1.665 mmol) was added at −78° C. and stirred for 10 mins. A solution of tert-butyl 3-benzyl-1-formyl-3,8-diazabicyclo[3.2.1]octane-8-carboxylate (Int-PP1) (500 mg, 1.51 mmol) in DME (10 mL) was added dropwise and stirred for 40 mins. The reaction was warmed to room temperature and stirred for 10 mins. Methanol (18.1 mL, 446 mmol) was added and the reaction was refluxed for an hour. The reaction was cooled to room temperature and concentrated in vacuo and partitioned into ethyl acetate and aqueous diluted HCl. The organic layer was separated, washed with saturated aqueous NaHCO and brine solution, dried over MgSO₄, filtered and excess solvent was removed under reduced pressure. The crude mixture was purified on a silica gel column using 0-10% MeOH in DCM to yield tert-butyl 3-benzyl-1-(cyanomethyl)-3,8-diazabicyclo[3.2.]octane-8-carboxylate (Int-PP2). MS (ESI) m/z [M+H]⁺ 342.

### Step C: tert-butyl 1-(cyanomethyl)-3,8-diazabicyclo[3.2.1]octane-8-carboxylate (Int-PP3

Ammonia (12 mL, 38.8 mmol) in methanol was added to a solution of tert-butyl 3-benzyl-1-(cyanomethyl)-3,8-diazabicyclo[3.2.1]octane-8-carboxylate (Int-PP2) (408 mg, 1.20 mmol) in methanol (12.0 mL). The flask was alternately purged with N2 and evacuated three times and palladium on carbon (15.00 mg, 0.014 mmol) was added. The N2 line was switched to a H₂ gas balloon and the flask alternately flushed with H₂ and vacuum twice and then H₂ was allowed to flow freely. The flask was heated to 40° C. and the reaction stirred under H₂ for 7 h. The reaction was filtered through a bed of CELITE and the filter bed was washed with fresh methanol. The filtrate was concentrated under reduced pressure and quickly passed through a silica gel filter pad, eluting with 50 mL (10% MeOH in DCM). The filtrate was concentrated and vacuum dried to give racemic tert-butyl 1-(cyanomethyl)-3,8-diazabicyclo[3.2.1]octane-8-carboxylate (Int-PP3). MS (ESI) m/z [M+H]⁺ 252.

### Step D: tert-butyl 14cyanomethyl)-3,8-diazabicyclo[3.2.1]octane-8-carboxylate (Int-PP4-1 and Int-PP4-2)

Racemic tert-butyl 1-(cyanomethyl)-3,8-diazabicyclo[3.2.1]octane-8-carboxylate (Int-PP3) was separated on a chiral SFC (Column G: Conditions: 15% MeOH w/0.1% NH₄OH) to give tert-butyl 1-(cyanomethyl)-3,8-diazabicyclo[3.2.1]octane-8-carboxylate (Int-PP4-1, Peak 1). MS (ESI) m/z [M+H]⁺ 252. ¹H NMR (500 MHz, DMSO-d₆) δ 4.03 (s, 1H), 2.90 (d, J=16.3 Hz, 1H), 2.78-2.65 (m, 3H), 2.47 (d, J=11.9 Hz, 2H), 2.11-1.99 (m, 1H), 1.92-1.74 (m, 2H), 1.75-1.62 (m, 1H), 1.43 (s, 9H). And tert-butyl 1-(cyanomethyl)-3,8-diazabicyclo[3.2.1]octane-8-carboxylate (Int-PP4-2, Peak 2). MS (ESI) m/z: [M+H]⁺ 252. ¹H NMR (500 MHz, DMSO-d₆) δ 4.03 (s, 1H), 2.90 (d, J=16.3 Hz, 1H), 2.71 (d, J=12.5 Hz, 3H), 2.50-2.41 (m, 2H), 2.12-2.01 (m, 1H), 1.82 (t, J=14.3 Hz, 2H), 1.74-1.63 (m, 1H), 1.43 (s, 9H).

### Intermediate O04: tert-butyl 1-(methoxynmethyl)-3,8-diazabicyclo[3.2.1]octane-8-carboxylate (Int-QQ4-1 and Int-OO4-2)

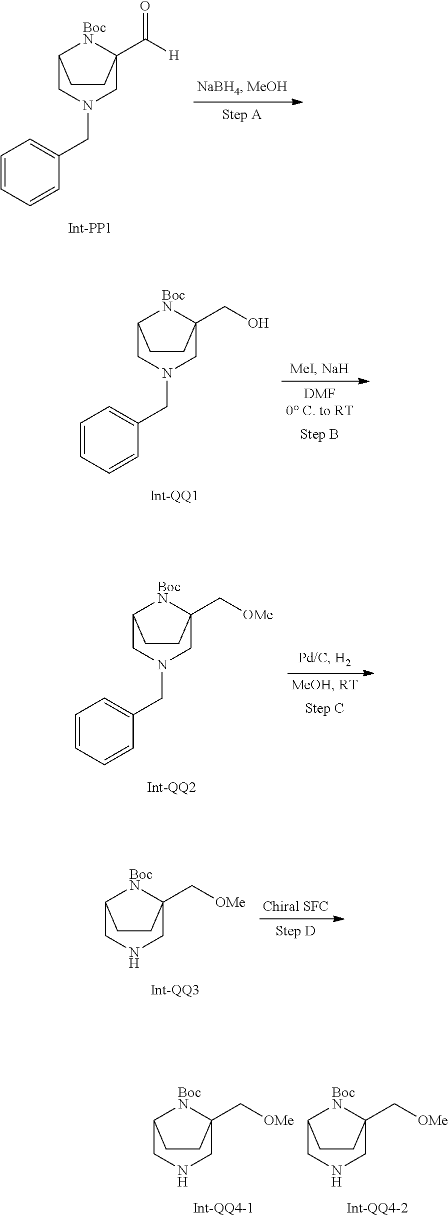

### Step A: tert-butyl 3-benzyl-1-(hydroxymethyl)-3,8-diazabicyclo[3.2.1]octane-8-carboxylate (Int-QQ1)

To tert-butyl 3-benzyl-1-formyl-3,8-diazabicyclo[3.2.1]octane-8-carboxylate (Int-PP1) (1g, 3.03 mmol) in methanol (36.1 mL, 893 mmol) cooled to 0° C., sodium borohydride (401 mg, 10.6 mmol) was added and the reaction was stirred for 1.5 hours. The reaction was quenched with cold water. Excess solvent was removed in vacuo and partitioned into ethyl acetate. The organic layer was separated, washed with brine, dried over MgSO₄, filtered and excess solvent was removed under reduced pressure. The residue obtained was purified on a silica gel column using 0-50% ethyl acetate in hexanes to give tert-butyl 3-benzyl-1-(hydroxymethyl)-3,8-diazabicyclo[3.2.1]octane-8-carboxylate (Int-QQ1). MS (ESI) m/z [M+H]⁺ 333.

### Step B: tert-butyl 3-benzyl-1-(methoxymethyl)-3,8-diazabicyclo[3.2.1]octane-8-carboxylate (Int-QQ2)

To a solution of tert-butyl 3-benzyl-1-(hydroxymethyl)-3,8-diazabicyclo[3.2.1]octane-8-carboxylate (Int-QQ1) (150 mg, 0.451 mmol) in DMF (2 mL) cooled to O ° C. was added NaH (21.66 mg, 0.541 mmol) and the reaction was stirred for 30 minutes. To the above stirred suspension, a solution of iodomethane (0.028 mL, 0.451 mmol) in DMF (0.25 mL) was added and the reaction was stirred overnight. The reaction mixture was cooled in ice water and quenched with cold water. The mixture was partitioned into ethyl acetate. The organic layer was separated, washed with brine, dried over MgSO₄, filtered and excess solvent was removed under reduced pressure. The crude residue obtained was purified on a silica gel column using 10% MeOH in DCM to give tert-butyl 3-benzyl-1-(methoxymethyl)-3,8-diazabicyclo[3.2.1]octane-8-carboxylate (Int-QQ2). MS (ESI) m/z [M+H]⁺ 347.

### Step C: tert-butyl 1-(methoxymethyl)-3,8-diazabicyclo[3,2,1]octane-8-carboxylate (Int-QQ3)

tert-Butyl 3-benzyl-1-(methoxymethyl)-3,8-diazabicyclo[3.2.1]octane-8-carboxylate (Int-QQ2) (80 mg, 0.231 mmol) was dissolved in methanol. The reaction mixture was alternately purged with N2 and vacuum a few times and then palladium on carbon (25 mg, 0.023 mmol) was added. The N₂ line was switched to a H₂ gas balloon and the flask alternately flushed with H₂ and vacuum twice and then H₂ was allowed to flow freely for four hours. Then, the reaction was filtered through a bed of CELITE. The filter bed was washed with fresh methanol. The filtrate was concentrated and purified by passing it through a silica gel filter pad and eluted with 50 mL (10% MeOH in DCM). The filtrate was concentrated and vacuum dried to give racemic tert-butyl 1-(methoxymethyl)-3,8-diazabicyclo[3.2.1]octane-8-carboxylate (Int-QQ3). MS (ESI) m/z [M+H]⁺ 257.

### Step D: tert-butyl 1-(methoxymethyl)-3,8-diazabicyclo[3.2.1]octane-8-carboxylate (Int-QQ4-1 and Int-QQ4-2)

Racemic tert-butyl 1-(methoxymethyl)-3,8-diazabicyclo[3.2.1]octane-8-carboxylate (Int-QQ3) was separated using chiral SFC (Column B, Conditions: Modifier: 10%/MeOH w/0.1% NH₄OH) to give tert-butyl 1-(methoxymethyl)-3,8-diazabicyclo[3.2.1]octane-8-carboxylate (In-QQ4-1, Peak 1). MS (ESI) m/z, [M+H]⁺ 257. ¹H NMR (499 MHz, DMSO-d₆) δ 4.00 (d, J=6.5 Hz, 1H), 3.71 (d, J=8.0 Hz, 1H), 3.51 (d, J=9.3 Hz, 1H), 3.25 (s, 3H), 2.76 (s, 1H), 2.67 (s, 1H), 2.61 (d, J=12.1 Hz, 1H), 2.54 (s, 1H), 1.91 (s, 1H), 1.80 (d, J=49.8 Hz, 2H), 1.65 (d, J=24.7 Hz, 1H), 1.41 (s, 9H), 1.25 (d, J=47.8 Hz, 1H). And tert-butyl 1-(methoxymethyl)-3,8-diazabicyclo[3.2.1]octane-8-carboxylate (Int-QQ4-2, Peak 2). MS (EST) m z [M+H]⁺ 257. ¹H NMR (499 MHz, DMSO-d₆) δ 3.97 (d, J =6.3 Hz, 1H), 3.69 (s, 1H), 3.49 (s, 1H), 3.25 (s, 3H), 2.73 (d, J=12.4 Hz, 1H), 2.67 (s, 1H), 2.56 (d, J=12.1 Hz, 1H), 2.47 (d, J=12.2 Hz, 1H), 1.89 (s, 1H), 1.77 (d, J=51.5 Hz, 2H), 1.63 (d, J=24.9 Hz, 1H), 1.40 (s, 9H), 1.26 (d, J=16.5 Hz, 1H)

### Intermediate RR1: tert-Butyl 1-(hydroxymethyl)-3,8-diazabicyclo[3.2.1]octane-8-carboxylate (Int-RR1-1 and Int-RR1-2)

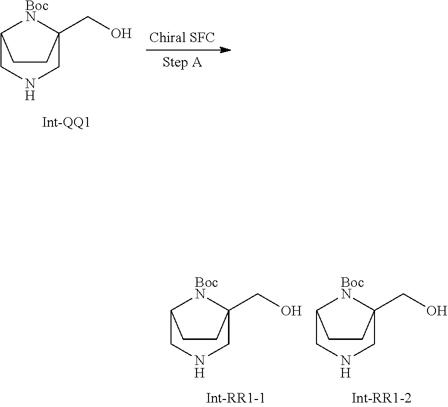

Racemic tert-butyl 3-benzyl-1-(hydroxymethyl)-3,8-diazabicyclo[3.2.1]octane-8-carboxylate (Int-QQ1) was resolved using chiral SFC (Column H; Conditions: 25% MeOH w/0.1% NH₄₀H) to give tert-butyl 1-(hydroxymethyl)-3,8-diazabicyclo[3.2.1]octane-8-carboxylate (Int-RR1-1, Peak 1). MS (ESI) m/z [M+Na]⁺ 265. ¹H NMR (499 MHz, DMSO-d₆) δ 4.78 (s, 1H), 3.99 (d, J=6.4 Hz, 1H), 3.71 (d, J=11.5 Hz, 1H), 3.50 (d, J=11.5 Hz, 2H), 2.70 (d, J=12.3 Hz, 2H), 2.56 (d, J=11.8 Hz, 1H), 2.47 (s, 1H), 1.96-1.80 (m, 1H), 1.76-1.56 (m, 3H), 1.40 (s, 9H), tert-Butyl 1-(hydroxymethyl)-3,8-diazabicyclo[3.2.1]octane-8-carboxylate (Int-RR1-2, Peak 2). MS (ESI) m/z [M+Na] 265. ¹H NMR (499 MHz, DMSO-d₆) δ 4.78 (s, 1H), 3.99 (d, J=6.3 Hz, 1H), 3.71 (d, J=11.5 Hz, 1H), 3.50 (d, J=11.5 Hz, 2H), 2.70 (d, J=12.3 Hz, 2H), 2.62-2.51 (m, 1H), 2.47 (s, 1H), 1.93-1.80 (m, 1H), 1.80-1.58 (m, 3H), 1.40 (s, 9H).

### Intermediate RS1: tert-butyl (1S,5S,6S)-6-hydroxy-3,8-diazabicyclo[3.2.1]octane-8-carboxylate (Int-RS1-1)

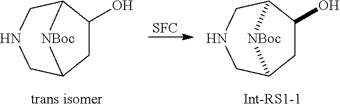

tert-Butyl 6-hydroxy-3,8-diazabicyclo[3.2.1]octane-8-carboxylate (trans isomer) (1000 mg, 4.38 mmol. Advanced ChemBlocks Inc.) was sent for chiral SFC separation (Column P, Conditions: 20% MeOH with 0.1% NH₄OH) to yield tert-butyl (1S,5S,6S)-6-hydroxy-3,8-diazabicyclo[3.2.1]octane-8-carboxylate (Int-RS1-1, Peak 1). ¹H NMR (600 MHz, DMSO-d₆) δ 5.12 (s, 1H), 4.24 (s, 1H), 3.86-3.74 (m, 1H), 3.62 (s, 1H), 3.17 (s, 1H), 2.89 (s, 1H), 2.76 (s, 1H), 2.65 (t, J=14.4 Hz, 1H), 2.40-2.22 (m, 2H), 1.48 (d, J=12.0 Hz, 1H), 1.40 (s, 9H).

### Intermediate SS7-2: tert-butyl 7-hydroxy-1-methyl-3,8-diazabicyclo[3.2.1]octane-8-carboxylate (Int-SS7-2)

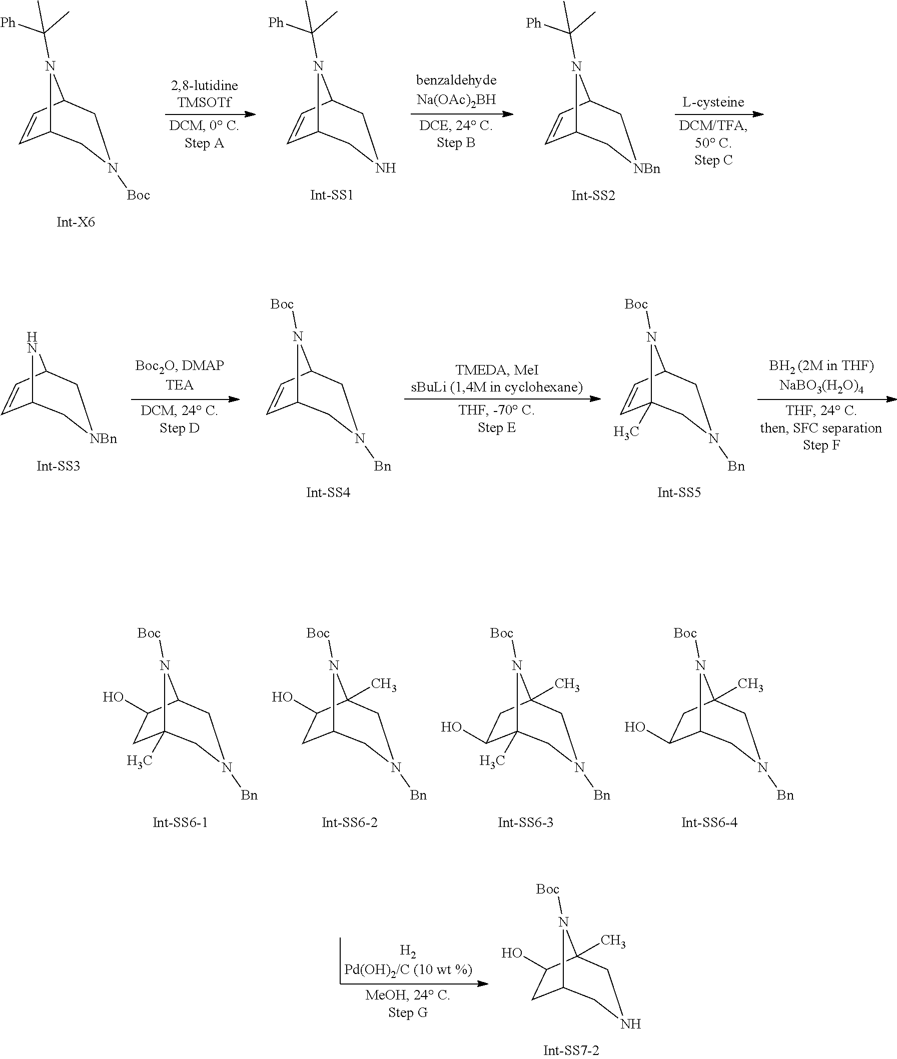

### Step A: 8-(2-phenylpropan-2-yl)-3,8-diazabictclo[3.2.1]oct-6-ene (t-SS1)

tert-butyl2-(2-phenypropan-2-yl)-3,8-diazabicyclo[3.2.1]oct-6-ene-3-carboxylate (Int-M6) (500 mmg, 1.522 mmol) was added in a 30 mL vial. The reaction vessel was evacuated and backfilled with a balloon of nitrogen three times. DCM (3806 μl) and 2,6-dimethylpyridine (1064 μl, 9.13 mmol) were added into the reaction vessel. The resulting mixture was cooled down to 0 TC. Trimethylsilyl trifluoromethanesulfonate (1102 μl, 6.09 mmol) was added dropwise into the reaction vessel. The resulting mixture was stirred for 1 h at 0° C. The product mixture was quenched with saturated sodium bicarbonate aqueous solution (5 mL). The quenched product mixture was extracted three times with dichloromethane (3*30 mL). The organic layers were dried and the dried solution was filtered. The filtrate was concentrated to dryness to yield 8-(2-phenylpropan-2-yl)-3,8-diazabicyclo[3.2.1]oct-6-ene (Int-SS1). MS (ESI) m/z [M+H]⁺ 229.

### Step B: 3-benzyl-8-(2-phenylpropan-2-yl)-3,8-diazabicyclo[3,2,1]oct-6-ene (Int-SS2)

8-(2-phenylpropan-2-yl)-3,8-diazabicyclo[3.2.1]oct-6-ene (Int-SS1) (347 mg, 1.52 mmol) was dissolved in DCE (3800 μl) in a 30 mL vial. Benzaldehyde (170 μl, 1.672 mmol) was added into the reaction vessel and the resulting mixture was stirred until all the starting material was dissolved. Sodium triacetoxyhydroborate (515 mg, 2.432 mmol) was added into the reaction vessel in one portion. The resulting mixture was stirred for 1.5 h at 24° C. The product mixture was quenched with saturated sodium bicarbonate aqueous solution (3 mL). The quenched product mixture was poured into a separatory funnel charged with saturated sodium carbonate aqueous solution (10 mL) and DCM (10 mL). The aqueous layer was extracted three times with DCM (3*30 mL). The organic layer was dried over sodium sulfate. The dried solution was filtered and the filtrate was concentrated to dryness. The residue obtained was purified by flash column chromatography with a 24-g silica gel column, eluting with hexanes initially, grading to 40% ethyl acetate-hexanes, linear gradient to afford the desired product 3-benzyl-8-(2-phenylpropan-2-yl)-3,8-diazabicyclo[3.2.1] oct-6-ene (Int-SS2). MS (ESI) m-: [M+H]⁺ 319.

### Step C: 3-benzyl-3,8-diazabicyclo[3.2.1]oct-6-ene

3-benzyl-8-(2-phenylpropan-2-yl)-3,8-diazabicyclo[3.2.1]oct-6-ene (Int-SS2) (500 mg, 1.570 mmol) and L-cysteine (571 mg. 4.71 mmol) were added in a 30 ml vial. The reaction vessel was evacuated and backfilled with a balloon of nitrogen three times. DCM (2617 μl) and TFA (2617 μl) were added into the reaction vessel. The resulting mixture was heated at 50° C. for 1.5h. The product mixture was cooled down to room temperature and then poured into saturated sodium carbonate aqueous solution (30 mL). After the mixture was thoroughly shook, the aqueous layer was basified to pH >14 with crushed sodium hydroxide solid. The aqueous layer was extracted three times with DCM (3*30 mL). The organic layers were dried over sodium sulfate. The dried solution was filtered and the filtrate was concentrated to dryness to yield 3-benzyl-3,8-diazabicyclo[3.2.1]oct-6-ene (Int-SS3) MS (ESI) m/z [M+H]⁺ 201.

### Step D: tert-butyl 3-benzyl-3,8-diazabicyclo[3.2.1]oct-6-ene-8-carboxylate (Int-SS4)

3-benzyl-3,8-diazabicyclo[3.2.1]oct-6-ene (Int-SS3) (314 mg, 1.57 mmol) was added into a 30 mL vial. DCM (3925 μl) and triethylamine (438 μl, 3.14 mmol) were added into the reaction vessel to dissolve the starting material. Di-tert-butyl dicarbonate (685 mg, 3.14 mmol) and N,N-dimethylpyridin-4-amine (19.18 mg, 0.157 mmol) were added into the reaction vessel. The resulting mixture was stirred for 1 h at 24° C. The product mixture was diluted with ether (1(0) mL) and washed three times with saturated sodium chloride aqueous solution (3*30 mL). The organic layer was dried over sodium sulfate. The dried solution was filtered and the filtrate was concentrated to dryness. The residue obtained was purified by flash column chromatography with a 40-g silica gel column, eluting with hexanes initially, grading to 40% ethyl acetate-hexanes, linear gradient to afford the desired product tert-butyl 3-benzyl-3,8-diazabicyclo[3.2.1]oct-6-ene-8-carboxylate (Int-SS4). 1H NMR (499 MHz, Chloroform-d) δ 7.28 (td, J=12,2, 6.8 Hz, 5H), 6.20 (d, J=17.5 Hz, 2H), 4.52 (d, J=51.9 Hz. 2H). 3.60 (s, 2H). 2.61 (d, J=10.7 Hz, 2H), 2.51-2.19 (m, 2H), 1.46 (s, 9H).

### Step E: tert-butyl 3-benzyl-1-methyl-3,8-diazabicyclo[3.2.1]oct-6-ene-8-carboxylate (Int-SS5)

tert-Butyl 3-benzyl-3,8-diazabicyclo[3.2.1]oct-6-ene-8-carboxylate (Int-SS4) (9.74 g, 32.4 mmol) was added to a round-bottomed flask. The reaction vessel was evacuated and backfilled with nitrogen 3 times and then THF (195 mL) was added. The reaction vessel was cooled to −70° C. TMEDA (5.48 mL, 36.3 mmol) was added into the reaction vessel. To this solution was added sec-butylthium (34.7 mL, 48.6 mmol, 1.4 M in cyclohexane). The mixture was stirred for 60 min then methyl iodide (3.04 mL, 48.6 mmol) was added neat. The product mixture was worked up by washing with saturated aqueous NaHCO₃ (100 mL), and the aqueous layer was extracted with EtOAc (2×200 mL). The combined organic layers were dried with MgSO₄, filtered, and the filtrate was evaporated to dryness. The residue obtained was purified by silica gel column chromatography with 0 to 100% EtOAc in hexane to afford tert-butyl 3-benzyl-1-methyl-3,8-diazabicyclo[3.2.1]oct-6-ene-8-carboxylate (Int-SS5). MS (ESI) m/z [M+H]⁺ 315.

### Step F: tert-butyl 3-benzyl-7-hydroxy-1-methyl-3,8-diazabicyclo[3.2.1]octane-8-carboxylate (Int-SS6-2)

tert-Butyl 3-benzyl-1-methyl-3,8-diazabicyclo[3.2.1]oct-6-ene-8-carboxylate (Int-SS5) (2.95 g, 9.38 mmol) was added to a 40 mL vial. The reaction vessel was evacuated and backfilled with a balloon of nitrogen three times. THF (10 mL) was added into the reaction vessel to dissolve the starting material. Borane (23.5 mL, 46.9 mmol, 2 M solution) in THF was added dropwise into the reaction vessel at 24° C. The resulting mixture was stirred for 16 h at room temperature. Water (21 mL) was added dropwise very carefully into the reaction vessel. After the gas evolution ceased, sodium perborate tetrahydrate (7.22 g, 46.9 mmol) was added into the reaction vessel in one portion. The resulting mixture was stirred for 3 h. The product mixture was extracted three times with DCM (3×30 mL) and three times with ether (3×30 mL). The combined organic layers were dried over sodium sulfate. The dried solution was filtered, and the filtrate was concentrated to dryness. The residue obtained was purified by flash column chromatography with a 24 g silica gel column, eluting with hexanes initially, grading to 80% ethyl acetate/hexanes, linear gradient to yield the racemic mixture. The diastereomeric mixture was submitted to two rounds of chiral SFC separation (Column 1: Column K: Conditions 1: 25% MeOH w/0.1% NH₄OH; Column 2: Column 1: Conditions 2: 20% MeOH w/0.1% NH₄OH) to yield tert-butyl 3-benzyl-7-hydroxy-1-methyl-3,8-diazabicyclo[3.2.1]octane-8-carboxylate (Int-SS6-2, Peak1/2, Column 1: Peak 2, Column 2). MS (ESI) m/z [M+H]⁺ 333.

### Step G: tert-butyl 7-hydroxy-1-methyl-3,8-diazabicyclo[3.2.1]octane-8-carboxylate (Int-SS7-2)

tert-Butyl 3-benzyl-7-hydroxy-1-methyl-3,8-diazabicyclo[3.2.1]octane-8-carboxylate (Int-SS6-2) (50 mg. 0.150 mmol) and palladium hydroxide (11 mg, 7.52 μmol) were added in a 30 mL vial. The reaction vessel was evacuated and backfilled with a balloon of nitrogen three times. MeOH (1.5 mL) was added into the reaction vessel. The reaction mixture was stirred at 24° C. for 4 h. The product mixture was thoroughly purged with nitrogen and filtered. The filtrate was concentrated to dryness to yield terl-butyl 7-hydroxy-1-methyl-3,8-diazabicyclo[3.2.1]octane-8-carboxylate (Int-SS3-2) which was used in the next step without purification. MS (EST) m/z [M+H]⁺ 243. The regiochemistry and relative stereochemistry for this intermediate and the intermediates in the table below were determined by NMR analysis. The absolute stereochemistry was not unequivocally determined.

| | | | [M + H]⁺ |
|---|---|---|---|
| Int | Structure | Compound Name | Found |
| Int-SS7-1 | 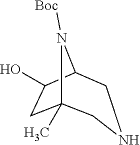 [SMILES: CC(C)(C)OC(=O)N1C2CNCC1(C)CC2O] | tert-butyl 6-hydroxy-1-methyl-3,8- diazabicyclo[3.2.1]octane-8-carboxylate | 243 |
| | Peak 1/2, Column 1; | | |
| | Peak 1, Column 2 | | |
| Int-SS7-3 | 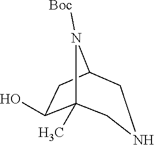 [SMILES: CC(C)(C)OC(=O)N1C2CNCC1(C)C(O)C2] | tert-butyl 7-hydroxy-1-methyl-3,8- diazabicyclo[3.2.1]octane-8-carboxylate | 243 |
| | Peak 3, Column 1 | | |
| Int-SS7-4 | 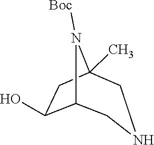 [SMILES: CC(C)(C)OC(=O)N1C2CNCC1(C)CC2O] | tert-butyl 6-hydroxy-1-methyl-3,8- diazabicyclo[3.2.1]octane-8-carboxylate | 243 |
| | Peak 4, Column 1 | | |

### Intermediate ST3: tert-butyl (1R,5S)-6-hydroxy-1,5-dimethyl-3,8-diazabicyclo[3.2.1]octane-8-carboxylate (Int-ST3)

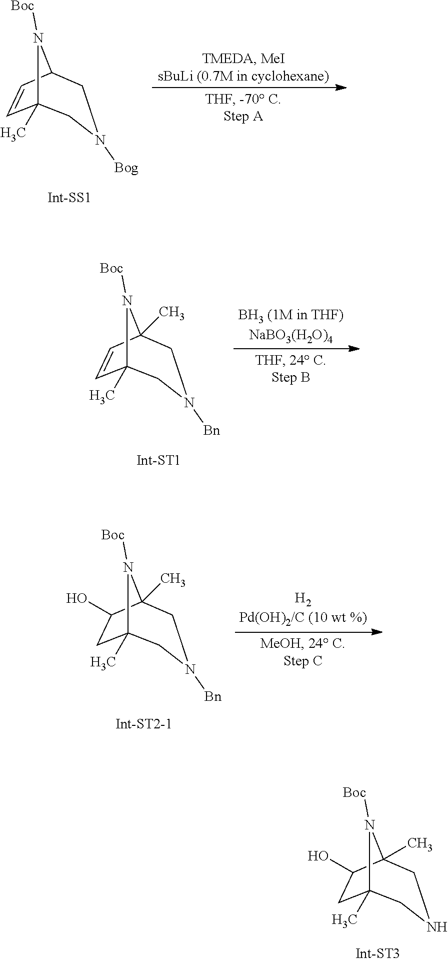

Step A: tert-butyl 3-benzyl-1.5-dimethyl-3,8-diazabicyclo[3.2.1]oct-6-ene-8-carboxylate (Int-ST1)

tert-Butyl 3-benzyl-1-methyl-3,8-diazabicyclo[3.2.1]oct-6-ene-8-carboxylate (Int-SS5) (7.19 g, 22.9 mmol) and tetramethylethanediamine (3.87 mL, 25.6 mmol) were added into a 1 L round-bottomed flask. The reaction vessel was evacuated and backfilled with a balloon of nitrogen three times. THF (152 mL) was added into the reaction mixture. The resulting solution was cooled down to −78° C. A 0.7 M solution of sec-butylthium (49.0 mL, 34.3 mmol) was cannulated into the reaction mixture over 15 min. The resulting mixture was stirred for 1 h at −78° C. Then iodomethane (2.15 mL, 34.3 mmol) was added dropwise. The resulting mixture was stirred for 10 min at −78° C. then gradually warmed up to 0° C. The product mixture was quenched with ammonium chloride aqueous solution (50 mL). The layers that formed were separated. The organic layer was dried over sodium sulfate. The dried solution was filtered and the filtrate was concentrated to dryness. The residue obtained was purified by flash column chromatography with a silica gel column, eluting with hexanes initially, grading to 50% ethyl acetate-hexanes, linear gradient to afford tert-butyl 3-benzyl-1,5-dimethyl-3,8-diazabicyclo[3.2.1]oct-6-ene-8-carboxylate (Int-ST1). MS (ESI): m/z (M+H)⁺ 329.

### Step B: tert-butyl (1R,5S)-3-benzyl-6-hydroxy-1.5-dimethyl-3,8-diazabicyclo[3.2.1]octane-8-carboxylate (Int-ST2-1)

tert-Butyl 3-benzyl-1,5-dimethyl-3,8-diazabicyclo[3.2.1]oct-6-ene-8-carboxylate (Int-ST1) (6.57 g, 20.0 mmol) was added in a 40 mL vial. The reaction vessel was evacuated and backfilled with a balloon of nitrogen three times. THF (22 mL) was added into the reaction vessel to dissolve the starting material. A 1 M THF solution of borane-THF adduct (100 mL, 100 mmol) was added dropwise into the reaction vessel at 24° C. The resulting mixture was stirred for 16 h at room temperature. Water (45 mL) was added dropwise very carefully into the reaction vessel. After the gas evolution ceased, sodium perborate tetrahydrate (15.4 g, 100 mmol) was added into the reaction vessel in one portion. The resulting mixture was stirred for 3 h. The product mixture was extracted three times with DCM (3×30 mL) and three times with ether (3×30 mL). The organic layer was dried over sodium sulfate. The dried solution was filtered and the filtrate was concentrated to dryness. The residue obtained was purified by flash column chromatography with a silica gel column, eluting with hexanes initially, grading to 80% ethyl acetate-hexanes, linear gradient. The material was further resolved by SFC (Column 1; Conditions: 10% methanol with 0.1% NH₄OH) to afford peak 1 as tert-butyl (1R,5S)-3-benzyl-6-hydroxy-1,5-dimethyl-3,8-diazabicyclo[3.2.1]octane-8-carboxylate (Int-ST2-1). MS (ESI): m/z (M+H)⁺ 347.

### Step C: tert-butyl (1R,5S)-6-hydroxy-1,5-dimethyl-3,8-diazabicyclo[3.2.1]octane-8-carboxylate (Int-ST3)

tert-Butyl (1R,5S)-3-benzyl-6-hydroxy-1,5-dimethyl-3,8-diazabicyclo[3.2.1]octane-8-carboxylate (Int-ST2-1) (100 mg, 0.289 mmol) and palladium on activated carbon (10% wt. 15 mg, 0.014 mmol) were added in a 30 mL vial. The reaction vessel was evacuated and backfilled with a balloon of nitrogen three times. 2,2,2-Trifluoroethanol (2.9 mL) was added into the reaction vessel. The reaction vessel was evacuated and backfilled with a balloon of nitrogen three times. The reaction vessel was evacuated and backfilled with a balloon of hydrogen three times. The reaction mixture was stirred for 4h. The product mixture was thoroughly purged with nitrogen and filtered. The filtrate was concentrated to dryness. The residue obtained containing tert-butyl (1R,5S)-6-hydroxy-1,5-dimethyl-3,8-diazabicyclo[3.2.1]octane-8-carboxylate (Int-ST3) was used directly in the next step without purification. MS (ESI): m/z (M+Na)⁺ 279.

### Intermediate SU2: tert-butyl (R,5S)-6-methoxy-1,5-dimethyl-3,8-diazabicyclo[3.2.1]octane-8-carboxylate (Int-SU2)

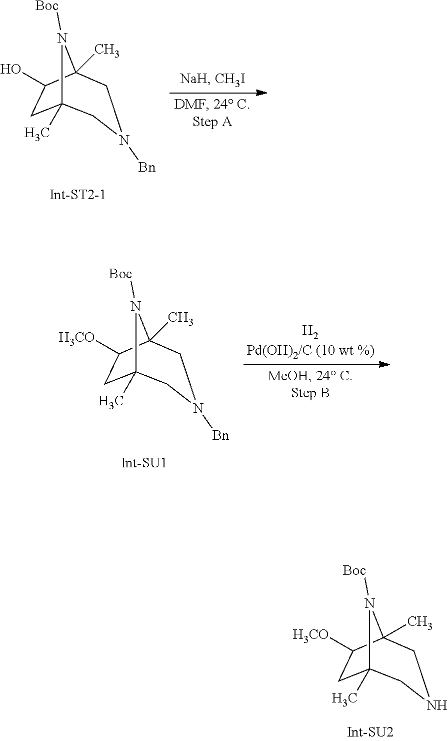 [SMILES: CC(C)(C)OC(=O)N1C2(C)CC(O)C1(C)CN(Cc1ccccc1)C2 ; COC1CC2(C)CN(Cc3ccccc3)CC1(C)N2C(=O)OC(C)(C)C ; COC1CC2(C)CNCC1(C)N2C(=O)OC(C)(C)C]

### Step B: tert-butyl (1R,5S)-6-methoxy-1.5-dimethyl-3,8-diazabicyclo[3.2.1]octane-8-carboxylate (Int-SU2)

tert-Butyl (1R,5S)-3-benzyl-6-methoxy-1,5-dimethyl-3,8-diazabicyclo[3.2.1]octane-8-carboxylate (Int-SU1) (500 mg, 1.39 mmol) and palladium on activated carbon (10% wt. 74 mg, 0.069 mmol) were added in a 30 mL vial. The reaction vessel was evacuated and backfilled with a balloon of nitrogen three times. Methanol (6.9 mL) was added into the reaction vessel. The reaction vessel was evacuated and backfilled with a balloon of nitrogen three times. The reaction vessel was evacuated and backfilled with a balloon of hydrogen three times. The reaction mixture was stirred for 4 h. The product mixture was thoroughly purged with nitrogen and filtered. The filtrate was concentrated to dryness. The residue obtained containing tert-butyl (1R,5,S)-6-methoxy-1,5-dimethyl-3,8-diazabicyclo[3.2.1]octane-8-carboxylate (Int-SU2) was used directly in the next step without purification. MS (ESI): m/z (M+Na)⁺ 293.

### Intermediate TT2: (R)-(1-((3-fluoropyrrolidin-1-1)methyl)cyclopropyl)methanol (Int-TT2)

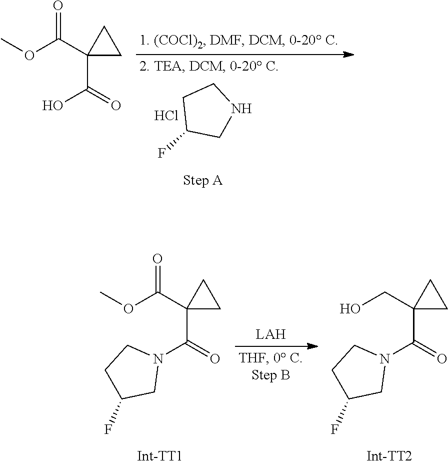

### Step A: Methyl (R)-1-(3-fluoropyrrolidine-1-carbonyl)cyclopropane-1-carboxylate (Int-TT1)

1-(Methoxycarbonyl)cyclopropane-1-carboxylc acid (100 g, 676 mmol) was dissolved in DCM (500 mL). The reaction mixture was cooled to 0° C. Oxalyl chloride (132 g, 1.04 mol) was added into the mixture at 0° C. followed by dropwise addition of DMF (2.54 g, 34.6 mmol) at 0° C. The resulting mixture was stirred for 3 h at 20° C. under N₂. The resulting mixture was concentrated under reduced pressure. The residue obtained was dissolved in DCM (160 mL) and added dropwise into a mixture of (R)-3-fluoropyrrolidine hydrochloride (86.5 g, 688 mmol) and TEA (209 g, 2.07 mol, 287 mL) in DCM (400 mL) at 0° C. The reaction mixture was allowed to warm up to 20° C. and stirred for 2 h under N₂. The product mixture was quenched with H₂O (1.50 L), extracted with DCM (800 mL×2). The organic layers were combined and washed with HCl (1 M, 500 mL), saturated aq. Na₂CO₃ (500 mL), and brine (500 mL). The organic laver was dried over Na₂SO₄ and filtered. The filtrate was evaporated under reduced pressure. The residue obtained was purified by column chromatography (SiO₂, Petroleum ether: Ethyl acetate=1: 0 to 5: 1) to afford methyl (R)-1-(3-fluoropyrrolidine-1-carbonyl)cyclopropane-1-carboxylate (Int-TT1). ¹H NMR (400 MHz, CDCl₃) δ 5.33 (t. J=3.48 Hz, 1H), 5.20 (t, J=3.42 Hz, 1H), 3.52-3.92 (m, 6H), 2.21-2.40 (m, 1H), 1.91-2.17 (m, 1H), 1.37-1.59 (m, 3H), 1.20-1,34 (m, 1H).

### Step B: (R)-(1-((3-fluoropyrrolidin-1-yl)methy)cyclopropylmethanol (Int-TT2)

The reaction vessel was charged with THF (500 mL) and purged with N₂. The reaction vessel was cooled to 0-5° C., and then charged with LiAlH₄ (54.6 g, 1.44 mol) in portions. A solution of methyl (R)-1-(3-fluoropyrrolidine-1-carbonyl)cyclopropane-1-carboxylate (Int-TT) (124 g, 576 mmol) in THF (250 mL) was added into the reaction vessel under N2 keeping the reaction temperature at 0-10° C. The reaction mixture was allowed to warm up to room temperature and stirred for 2 h at 25° C. The reaction mixture was cooled to 5-10° C. Water (54.6 mL), 15% aq. NaOH (54.6 mL) and water (54.6 mL) were added sequentially dropwise into the reaction mixture maintaining the internal temperature at 5-10° C. The quenched product mixture was filtered and the filter cake was washed with THF (200 mL×2). The filtrate was extracted with EtOAc (100 mL×3). The organic layers were combined and concentrated to dryness. The residue obtained was purified by column chromatography (SiO₂, Petroleum ether: Ethyl acetate=1:0 to 0:1) to afford (R)-(1-((3-fluoropyrrolidin-1-yl)methyl)cyclopropyl)methanol (Int-T2). ¹H NMR (400 MHz, CDCl₃) δ 5.05-5.24 (m, 1H), 3.48-3.63 (m, 2H), 2.89-3.05 (m, 2H), 2.71-2.86 (m, 1H), 2.49-2.67 (m, 3H), 1.94-2.20 (m, 2H), 0.45-0.57 (m, 2H), 0.31-0.42 (m, 2H).

### Intermediate UU8: (R)-(1-((dimethylamino)methyl)-2,2-difluorocyclopropyl)methanol

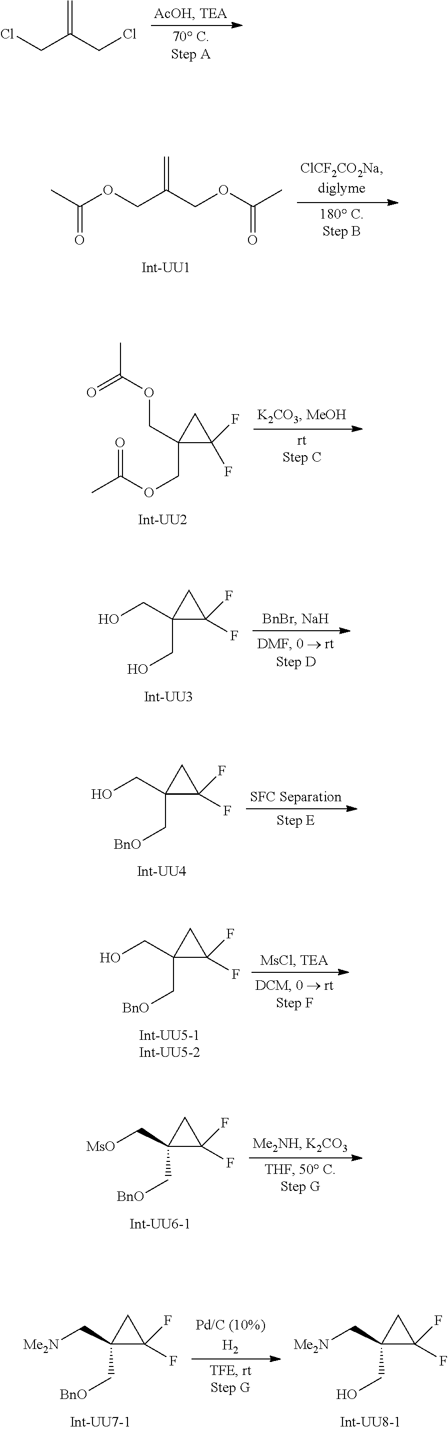

### Step A: 2-[(acetyloxy)methyl]prop-2-en-1-yl acetate (Int-UU1)

A 5L 4-necked round-bottom flask was charged with 3-chloro-2-(chloromethyl)prop-1-ene (600 g, 4.80 mol), triethylamine (1.46 kg, 14.40 mol), and acetic acid (721 g, 12.0 mol). The resulting solution was stirred overnight at 70° C. The reaction mixture was cooled to room temperature and quenched by the addition of 3 L of water. The resulting solution was extracted with ethyl acetate (3×1 L) and the combined organic layers were washed with brine solution (2×1 L). The organic layers were dried over anhydrous sodium sulfate. The dried solution was filtered and the filtrate was concentrated. The residue was purified by silica gel column with ethyl acetate/petroleum ether (1:6) to provide 2-[(acetyloxy)methyl]prop-2-en-1-yl acetate (Int-UU1).

### Step B: [1-[(acetyloxy)methyl]-2,2-difluorocyclopropyl]methyl acetate (Int-UU2)

Into a 20-L 4-necked round-bottom flask and maintained with an inert atmosphere of nitrogen was placed a solution of 2-[(acetyloxy)methyl]prop-2-en-1-yl acetate (Int-UU1) (6(0) g, 3.48 mol) in diglyme (5 L). This was followed by the addition of a solution of ClCF₂CO₂Na (2.65 kg, 17.4 mol) in diglyme (5 L) dropwise with stirring at 180° C., over 5 h. The resulting solution was stirred for 1 h at 180° C. The reaction mixture was cooled to room temperature and quenched by the addition of H₂O (5 L). The resulting solution was extracted with petroleum ether (4×2 L) and the organic layers were combined. The combined organic layers were washed with water (3×2 L) and dried over anhydrous sodium sulfate. The dried solution was filtered and the filtrate was concentrated to dryness to afford [l-[(acetyloxy)methyl]-2,2-difluorocyclopropyl]methyl acetate (Int-U U2), which was used directly into the next step without purification.

### Step C: 12.2-difluoro-1-(hydroxymethyl)evclopropyl)methanol (Int-UU3)

Into a 20-L 4-necked round-bottomed flask were placed [1-[(acetyloxy)methyl]-2,2-difluorocyclopropyl]methyl acetate (Int-UU2) (800 g, 3.60 mol), MeOH (10 L) and K₂CO₃ (995 g, 7.20 mol). The resulting solution was stirred overnight at room temperature. The solids were filtered out. The filtrate was concentrated. The resulting mixture was then diluted by the addition of water (2 L). The resulting solution was extracted with ethyl acetate (5×1 L). The organic layers were combined and dried over anhydrous sodium sulfate. The dried solution was filtered and the filtrate was concentrated under vacuum. The residue was purified by silica gel column chromatography with ethyl acetate/petroleum ether (1:1) to afford [2,2-difluoro-1-(hydroxymethyl)cyclopropyl]methanol (Int-UU3). ¹H NMR (300 MHz, DMSO-d₆) δ 1.30 (t, J=8.8 Hz, 2H), 3.52 (m, 4H), 4.79 (t, J=5.6 Hz, 2H).

### Step D: (1-((benzyloxy)methyl)-2,2-difluorocyclonropyl)methanol (Int-UU4)

A 500 mL single neck round bottom flask fitted with a pour-through nitrogen adapter was purged with nitrogen and then charged with sodium hydride (4.52 g, 113 mmol) and N,N-dimethylformamide (100 mL). The suspension was cooled to 0° C. Solid [2,2-difluoro-1-(hydroxymethyl)cyclopropyl]methanol (Int-UU3) (12.0 g, 87 mmol) was added portion-wise. The mixture was stirred while warming to rt for 1 h. The resultant reaction mixture was cooled to 0° C. and treated with a solution of benzyl bromide (10.3 mL, 87 mmol) in N,N-dimethylformamide (10 mL). The mixture was stirred at rt for 1 h and then treated with saturated aqueous ammonium chloride (10 mL) and water (10 mL). The mixture was partitioned between ethyl acetate (75 mL) and water (75 mL). The organic layer was washed with 1 wt % aqueous LiCl (30 mL×3), dried with anhydrous sodium sulfate, filtered and the filtrate was concentrated. Purification by column chromatography on silica gel (220 g, 0 to 40% EtOAc/hexanes) afforded (1-((benzyloxy)methyl)-2,2-difluorocyclopropyl)methanol (Int-UU4). 1H NMR (499 MHz, Methanol-d₆) δ 7.39-7.32 (m, 4H), 7.32-7.26 (m, 1H), 4.62-4.49 (m, 2H), 3.79-3.64 (m, 3H), 3.60 (dd, J=10,4, 2.1 Hz, 1H), 1.35 (dddd, J=29.1, 12.5, 8.0, 4.5 Hz, 2H).

### Step E: (1-((benzyloxy)methyl)-2,2-difluorocyclopropyl)methanol (Int-UU5-t)

Racemic (1-((benzyloxy)methyl)-2,2-difluorocyclopropyl)methanol (Int-UU4) was resolved using SFC chiral chromatography (Column B; Conditions: 5% MeOH w/0.1% NH₄OH and 5% H₂O) to yield (R)-(I-((benzyloxy)methyl)-2,2-difluorocyclopropyl)methanol (Int-UU8-1, Peak 1). 1H NMR (499 MHz, Methanol-d₆) δ 7.39-7.26 (m, 5H), 4.58-4.51 (m, 2H), 3.78-3.67 (m, 3H), 3.60 (dd, J=10,4, 2.0 Hz, 1H), 1.35 (dddd, J=28,4, 12.5, 8.0, 4.5 Hz, 2H). (S)-(1-((benzyloxy)methyl)-2,2-difluorocyclopropyl)methanol (Int-UU8-2, Peak 2) was also isolated.

### Step F: S)-(1-((benzyloxy)methyl)-2,2-difluorocyclopropyl)methyl methanesulfonate (Int-UU6-1)

(1-((benzyloxy)methyl)-2,2-difluorocyclopropyl)methanol (Int-UU5-t) (3.25 g, 14.24 mmol) in DCM (30 mL) was cooled to 0° C. and treated with triethylamine (7.94 mL, 57.0 mmol) and then a solution of methanesulfonyl chloride (2.22 mL, 28.5 mmol) in DCM (2.2 mL). The reaction mixture was stirred while warming to rt over 3 h. The reaction mixture was purified by column chromatography on silica gel (0 to 100% EtOAc/hexanes) to afford (S)-(1-((benzyloxy)methyl)-2,2-difluorocyclopropyl)methyl methanesulfonate (Int-UU6-11). MS (EST): mwz (M+Na) 329.

### Step G: (R)-1-(1-((benzyloxy)methyl)-2,2-difluorocyclopropyl)-N,N-dimethylmethanamine (Int-UU7-1)

(S)-(1-((benzyloxy)methyl)-2,2-difluorocyclopropyl)methyl methanesulfonate (Int-UU6-1) (4.10 g, 13.4 mmol) and dimethylamine (2 M in THF) (33.5 mL, 66.9 mmol) were treated with potassium carbonate (3.70 g, 26.8 mmol). The flask was capped and heated at 50° C. for 24 h. The reaction mixture was cooled to rt, diluted with water (20 mL), and extracted with ethyl acetate. The combined organic layers were dried with anhydrous sodium sulfate, filtered and the filtrate was concentrated. The crude residue was purified by column chromatography on silica gel (120g, 0 to 100% [1:3 EtOH/EtOAc]/hexanes) to afford (R)-1-(1-((benzyloxy)methyl)-2,2-difluorocyclopropyl)-N,N-dimethylmethanamine (Int-UU7-1). MS (ESI): m/z (M+H)⁺ 256.

### Step H: (R)-(1-((dimethylamino)methyl-2,2-difluorocyclopropy,Imethanol (Int-UU8-1)

(R)-1-(1-((benzyloxy)methyl)-2,2-difluorocyclopropyl)-N,N-dimethylmethanamine (Int-UU7-1) (2.96 g, 11.6 mmol) and Pd/C (10 wt %, wet support) (0.618 g, 0.580 mmol) in 2,2,2-trifluoroethanol (20.0 mL) were charged in a 100 mL recovery flask and stirred at rt under hydrogen gas (1 atm, balloon) for 20 h. The mixture was filtered through a pad of CELITE and the pad was rinsed with methanol (3×10 mL). The combined filtrate and washings were treated with 3M HCl in methanol (12 mL. 36.0 mmol) and then concentrated in vacuo to afford a clear, colorless viscous syrup. Diethyl ether (10 mL) was added and the mixture agitated to initiate precipitation. The mixture was concentrated in vacuo and then treated with diethyl ether (10 mL) and sonicated for 1 minute. The diethyl ether was decanted. The solid was dried under vacuum to afford (R)-(I-((dimethylamino)methyl)-2,2-difluorocyclopropyl)methanol (Int-UU8-1). MS (ESI): m z (M+H)⁺ 166.

(S)-(1-((dimethylamino)methyl)-2,2-difluorocyclopropyl)methanol (Int-UU8-2) was synthesized via a similar reaction sequence as above using Int-UU5-2 as an intermediate. MS (ESI): m/z (M+H)⁺ 166.

### Intermediate WW3: (1S,5S)-1-(((tert-butyldiphenylsilyl)oxy)methyl)-N,N-dimethylbicyclo[3.1.0]hexan-2-amine (Int-WW3)

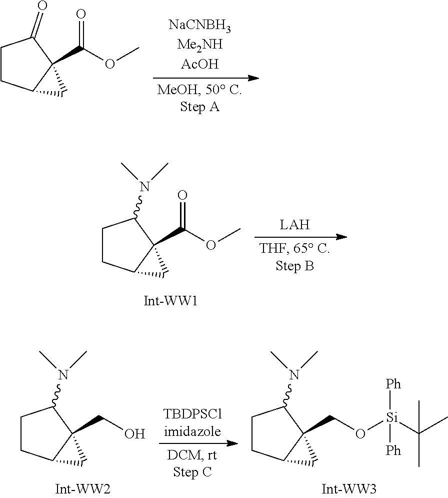 [SMILES: COC(=O)[C@@]12C[C@@H]1CCC2=O ; COC(=O)[C@@]12C[C@@H]1CCC2N(C)C ; CN(C)C1CC[C@H]2C[C@@]12CO ; CN(C)C1CC[C@H]2C[C@@]12CO[Si](c1ccccc1)(c1ccccc1)C(C)(C)C]

### Step A: methyl (1S,5S)-2-(dimethylamino)bicyclo[3.1.0]hexane-1-carboxylate (Int-WW1)

To methyl (1S,5S)-2-oxobicyclo[3.1.0]hexane-1-carboxylate (500, mg, 3.24 mmol) in MeOH (10.00 mL) was added dimethylamine (0.493 mL, 3.89 mmol) and acetic acid (0.186 mL, 3.24 mmol). The reaction mixture was stirred for 30 min at room temperature. To this mixture sodium cyanoborohydride (510 mg, 8.11 mmol) was added and the reaction was stirred at 50° C. overnight. The excess solvent was removed under reduced pressure and the crude material was partitioned into ethyl acetate and water. The aqueous layer was extracted twice, the organic layers were combined, washed with brine, dried over MgSO₄, filtered and excess solvent was removed under reduced pressure to afford methyl (1S,5S)-2-(dimethylamino)bicyclo[3.1.0]hexane-1-carboxylate (Int-WW1). MS (ESI): m z (M+H)⁺ 184.

### Step B: ((1S,5S)-2-(dimethylamino)bicyclo[3.1.0]hexan-1-yl)methanol (Int-WW2)

To a flask flushed with nitrogen, THF (2.00 mL) was added and the mixture was cooled to 0° C. LAH (2.087 mL, 2.087 mmol, 1 M THF) was added, followed by the dropwise addition of methyl (1S,5S)-2-(dimethylamino)bicyclo[3.1.0]hexane-1-carboxylate (Int-WW1) (255 mg, 1.39 mmol) in 1 mL THF. The reaction mixture was stirred for 5 min at 0° C., warmed to 65° C. and stirred for 2 h. The reaction was cooled to 0° C. and sodium sulfate decahydrate was added and the mixture was allowed to stir for 30 min. The reaction mixture was filtered and the filtrate was washed with fresh ethyl acetate two times. The combined organic layers were dried over anhydrous MgSO₄, filtered and excess solvent was removed under reduced pressure to yield ((1S,5S)-2-(dimethylamino)bicyclo[3.1.0]hexan-1-yl)methanol (Int-WW2) which was used in the next step without further purification.

### Step C: (1S,5S)-1-(((tert-butyldiphenylsilyl)oxy)methyl)-N,N-dimethylbicyclo[3.1.0]hexan-2-amine (Int-WW3)

To a solution of_(1S,5S)-2-(dimethylamino)bicyclo[3.1.0]hexan-1-yl)methanol (Int-WW2) (from the previous step) in 1 mL DCM at 0° C. were added imidazole (189 mg, 2.78 mmol) and TBDPS-C₁ (0.429 mL, 1.67 mmol) and the reaction was allowed to stir for 2.5 hours. The reaction mixture was partitioned into ethyl acetate and brine, the organic layer separated, dried over anhydrous MgSO₄, filtered and excess solvent was removed under reduced pressure. The crude residue was purified on a silica gel column using 10% DCM-MeOH to give (1S,5S)-1-(((tert-butyldiphenylsilyl)oxy)methyl)-N,N-dimethylbicyclo[3.1.0]hexan-2-amine (Int-WW3). MS (ESI): m/z (M+H)⁺ 394.

### Intermediate XX3: 2-(5.6-dichloro-4-methylpyridin-2-yl)isoindoline-1,3-dione

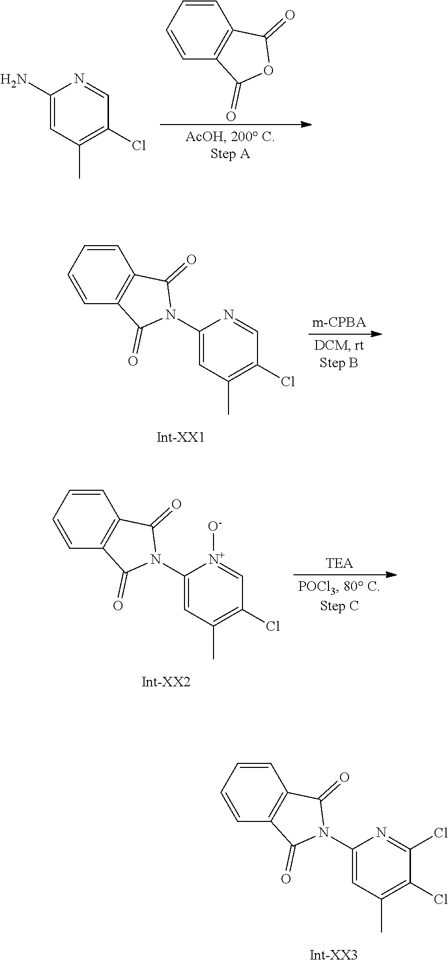

### Step A: 2-(5-chloro-4-methylyridin-2-yl)isoindoline-1,3-dione (Int-XX1)

To a flask containing 5-chloro-4-methylpyridin-2-amine (5.00 g, 35.1 mmol) were added acetic acid (70.1 mL) followed by phthalic anhydride (5.19 g, 35.1 mmol). The mixture was heated to 200° C. overnight. Upon cooling to room temperature, the mixture was carefully quenched with saturated aqueous sodium bicarbonate to a pH near neutral followed by the addition of ethyl acetate. The layers were separated and the water layer was re-extracted with ethyl acetate. The combined organic layers were then dried over magnesium sulfate, filtered and concentrated under reduced pressure. The resulting mixture was then purified by column chromatography (0-20% ethyl acetate gradient in hexanes) to afford 2-(5-chloro-4-methylpyridin-2-yl)isoindoline-1,3-dione (Int-XX1). MS (ESI): m/z (M+H)⁺ 273.

### Step B: 5-chloro-2-(1,3-dioxoisoindolin-2-yl)-4-methylpyridine 1-oxide (Int-X.X2

To a flask containing 2-(5-chloro-4-methylpyridin-2-yl)isoindoline-1,3-dione (Int-XX1) (1.00 g, 3.67 mmol) was added CH₂Cl₂ (18.3 mL) and then m-CPBA (0.822 g, 3.67 mmol). The mixture was allowed to stir overnight. The mixture was diluted with ethyl acetate and saturated aqueous sodium bicarbonate. The organic layer was separated, dried over magnesium sulfate, filtered and concentrated under reduced pressure. The resulting mixture was purified by silica gel column chromatography (0-100% ethyl acetate in hexanes, then 0-10% methanol in DCM) to afford 5-chloro-2-(1,3-dioxoisoindolin-2-yl)-4-methylpyridine 1-oxide (Int-XX2). MS (ESI): m/z (M+H)⁺ 289.

### Step C: 2-(5.6-dichloro-4-methylpyridin-2-yl)isoindoline-1,3-dione (Int-XX3)

To a flask containing 5-chloro-2-(1,3-dioxoisoindolin-2-yl)-4-methylpyridine 1-oxide (Int-XX2) (636 mg, 2.20 mmol) were added POCl₃(II mL) followed by triethylamine (307 μl. 2.20 mmol). The mixture was heated to 80° C. Once the reaction reached 80° C., the reaction was cooled to room temperature, the mixture was poured into a flask containing ice and saturated aqueous sodium bicarbonate. To the mixture were added ethyl acetate and then saturated aqueous sodium bicarbonate until the pH˜7. The organic layer was separated, dried over magnesium sulfate, filtered and concentrated under reduced pressure. The resulting mixture was purified by silica gel chromatography to afford 2-(5,6-dichloro-4-methylpyridin-2-yl)isoindoline-1,3-dione (Int-XX3). MS (ESI): m/z (M+H)⁺ 307, 309.

Int-YY3 below was made according to a similar procedure, using 4-methyl-5-(trifluoromethyl)pyridin-2-amine as starting material.

| | | | [M + H]⁺ |
|---|---|---|---|
| Int | Structure | Compound Name | Found |
| Int-YY3 | 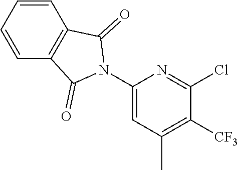 [SMILES: Cc1cc(N2C(=O)c3ccccc3C2=O)nc(Cl)c1C(F)(F)F] | 2-(6-chloro-4-methyl-5- (trifluoromethyl)pyridin-2- yl)isoindoline-1,3-dione | 341 |

### Intermediate ZZ2: 4-bromo-6-chloro-5-methyl-14triisopropylsilyl)-1H-indole (Int-ZZ2)

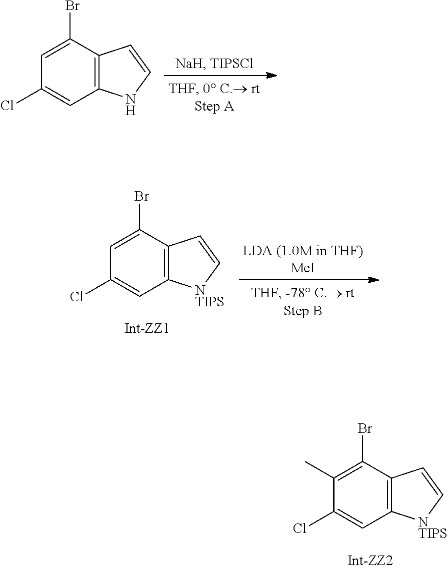

### Step A: 4-bromo-6-chloro-1-(triisopropylsilyl)-1H-indole (Int-ZZ1)

To a stirred solution of 4-bromo-6-chloro-1H-indole (900 mg, 3.90 mmol) in THF (8.00 mL), cooled to 0° C., NaH (195 mg, 4.88 mmol) was added and the reaction was stirred for 30 min. Triisopropylsilyl chloride (1.03 mL. 4.88 mmol) was added at 0° C. The mixture was stirred for 5 min and then warmed to room temperature and stirred for 2.5 h. The reaction mixture was quenched with cold water and diluted with ethyl acetate. The organic layer was extracted, washed with brine, dried over MgSO₄, filtered and excess solvent was removed under reduced pressure. The crude residue was purified on a silica gel column using 40% ethyl acetate/hexane to yield 4-bromo-6-chloro-1-(triisopropylsilyl)-1H-indole (Int-ZZ1). 1H NMR (499 MHz, DMSO-d₆) δ 7.61-7.48 (m, 2H), 7.40 (d, J=1 Hz, 1H), 6.63 (d, J=3 Hz, 1H), 1.75 (hept, J=7 Hz. 3H). 1.08 (d, J =7 Hz, 18H).

### Step B: 4-bromo-6-chloro-5-methyl-1-(triisopropylsilyl)-1H-indole (Int-ZZ2)

A solution of 4-bromo-6-chloro-1-(triisopropylsilyl)-1H-indole (Int-ZZ1) (350, mg, 0.905 mmol) in THF (4.00 mL) was cooled to −78° C. To this stirred solution, lithium diisopropylamide (1,357 mL, 1.357 mmol, 1.0 M in THF) was added. The mixture was stirred at −78° C. for 30 min. Iodomethane (0.085 mL, 1.4 mmol) was added and the reaction was stirred at −78° C. for 10 min and then warmed to room temperature and stirred for 2 h. The reaction mixture was quenched with saturated aqueous NH₄Cl and partitioned into ethyl acetate. The aqueous layer was separated, the organic layer was washed with brine, dried over MgSO₄, filtered and excess solvent was removed under reduced pressure. The crude residue was purified on a silica gel column using ethyl acetate in hexanes (0-5%) to yield 4-bromo-6-chloro-5-methyl-1-(triisopropylsilyl)-1H-indole (Int-ZZ2). MS (ESI): m % z (M+Na)⁺ 422, 424.

### Intermediate AAA5: 4-bromo-5-chloro-6-methyl-1-(tetrahydro-2H-pyran-2-yl)-1H-indazole (Int-AAA5)

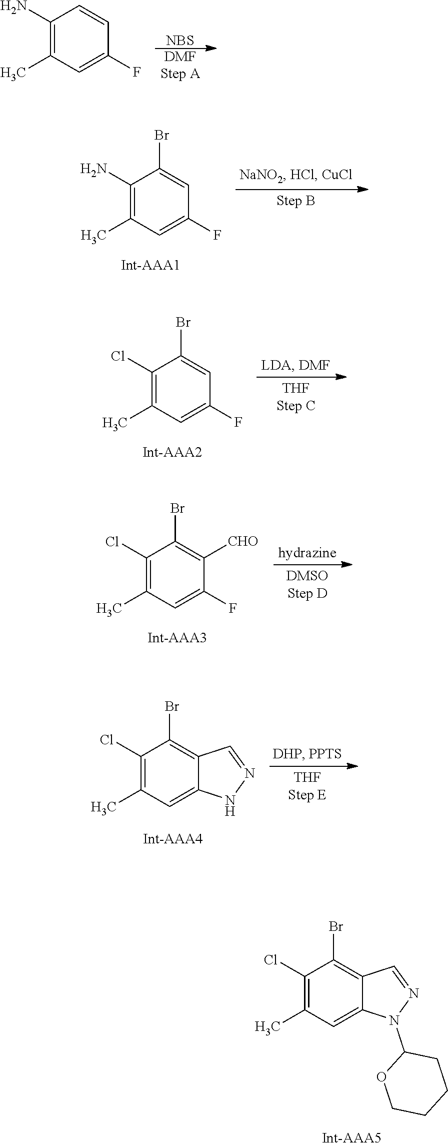

### Step A: 2-bromo-4-fluoro-6-methylaniline (Int-AAA1)

Into a 5-L 3-necked round-bottom flask were placed 4-fluoro-2-methylaniline (200 g, 1.60 mol) and DMF (2.00 L). This was followed by the addition of NBS (299 g, 1.69 mol) in portions at 0° C. The resulting solution was stirred for 1 h at room temperature. The reaction was then quenched by the addition of 10 L of water/ice. The resulting solution was extracted with ethyl acetate (2×2 L) and the organic layers combined. The resulting mixture was washed with brine (2 L). The organic layer was concentrated under vacuum. The residue was purified by silica gel column with ethyl acetate/petroleum ether (1:5) to afford 2-bromo-4-fluoro-6-methylaniline (Int-AAA1). MS (ESI): m/z (M+H)⁺ 204/206.

### Step B: 1-bromo-2-chloro-5-fluoro-3-methylbenzene (Int-AAA2)

Into a 20-L 4-necked round-bottom flask were placed 2-bromo-4-fluoro-6-methylaniline (Int-AAA1) (245 g, 1.20 mol), concentrated HCl (2.50 L), and water (2.50 L). The resulting solution was stirred for 1 h at 65° C. This was followed by the addition of a solution of sodium nitrite (99.4 g, 1.44 mol) in H₂O (1 L) dropwise with stirring at 0° C. over 30 min. The resulting solution was stirred for 20 min at 0° C. To this was added a solution of cuprous chloride (178 g, 1.80 mol) in concentrated HCl (1.5 L) at 0° C. The resulting solution was allowed to stir for 30 min at 70° C. The resulting solution was extracted with DCM (2×4 L) and the organic layers combined. The resulting mixture was washed with brine (3 L). The mixture was dried over anhydrous sodium sulfate and concentrated under vacuum to afford 1-bromo-2-chloro-5-fluoro-3-methylbenzene (Int-AAA2) which was used in subsequent steps without further purification.

### Step C: 2-bromo-3-chloro-6-fluoro-4-methylbenzaldehyde (Int-AAA3)

Into a 10-L 4-necked round-bottom flask were placed 1-bromo-2-chloro-5-fluoro-3-methylbenzene (Int-AAA2) (200, g, 895 mmol) and THF (2.50 L). This was followed by the addition of LDA (843 mL. 1.35 mol, 1.6 M in THF) dropwise with stirring at −78° C. over 1 h. The resulting solution was stirred for 1 h at −78° C. To this was added dimethylformamide (98.1 g, 1.34 mol) at −78° C. The resulting solution was allowed to stir for 30 min at −78° C. The reaction was then quenched by the addition of water/ice. The resulting solution was extracted with ethyl acetate (2×3 L) and the organic layers combined. The resulting mixture was washed with brine (3 L). The mixture was dried over anhydrous sodium sulfate and concentrated under vacuum to afford 2-bromo-3-chloro-6-fluoro-4-methylbenzaldehyde (Int-AAA3). 1H NMR (500 MHz, CDCl₃) S=10.29 (s, 1H), 7.09 (m, J=10.4 Hz, 1H), 2.51 (s, 3H).

### Step D: 4-bromo-5-chloro-6-methyl-1H-indazole (Int-AAA4)

Into a 10-L 4-necked round-bottom flask were placed 2-bromo-3-chloro-6-fluoro-4-methylbenzaldehyde (Int-AAA3) (250 g, 994 mmol), DMSO (5.00 L), and hydrazine (382 g, 11.9 mol). The resulting solution was stirred for 2 h at 130° C. The reaction was then quenched by the addition of water/ice. The solids were collected by filtration to afford 4-bromo-5-chloro-6-methyl-1H-indazole (Int-AAA4) and the material was used in the subsequent step without further purification.

### Step E: 4-bromo-5-chloro-6-methyl-1-(tetrahydro-2H-pyran-2-yl)-1H-indazole (Int-AAA5)

Into a 3-L 3-necked round-bottom flask, was placed 4-bromo-5-chloro-6-methyl-1H-indazole (Int-AAA4) (80.0 g, 326 mmol), THF (1.20 L), DHP (82.2 g, 976 mmol), and PPTS (8.19 g, 32.6 mmol). The resulting solution was stirred for 18 h at 50° C. The reaction was then quenched by the addition of water/ice. The resulting solution was extracted with ethyl acetate (2-2 L) and the organic layers were combined and dried over anhydrous sodium sulfate and concentrated under vacuum. The residue was purified by silica gel column with ethyl acetate/petroleum ether (1:50) to afford 4-bromo-5-chloro-6-methyl-1-(tetrahydro-2H-pyran-2-yl)-1H-indazole (Int-AAA5). ¹H NMR (300 MHz, CDCl₃): δ 7.95 (d, J=1 Hz, 1H), 7.45 (t, J=1 Hz, 1H), 5.68 (dd, J=9, 3 Hz, 1H), 4.00 (d, J=12 Hz, 1H), 3.84-3.69 (m, 1H), 2.58 (d, J=1 Hz, 3H), 2.55-2.46 (m, 1H), 2.26-2.04 (m, 2H), 1.90-1.63 (m, 3H).

### Intermediate BBB6: 4-bromo-6-chloro-5-methyl-1-(tetrahydro-2H-pyran-2-yl)-1H-indazole (Int-BBB6)

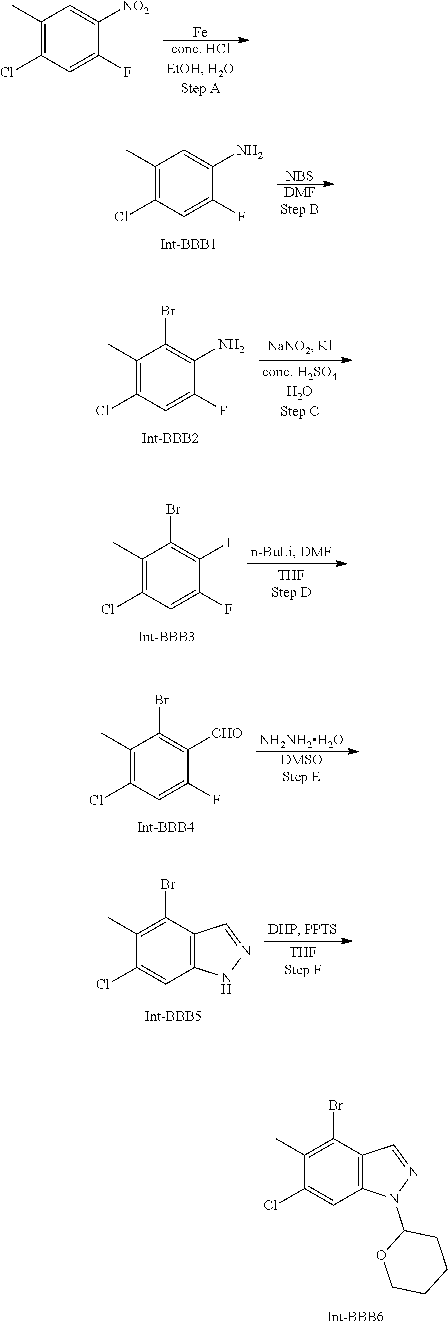

### Step A: 4-chloro-2-fluoro-5-methylaniline (Int-BBB1)

Into a 5-L 4-necked round-bottom flask purged and maintained with an inert atmosphere of nitrogen were placed 1-chloro-5-fluoro-2-methyl-4-nitrobenzene (210, g, 1.11 mol), EtOH (1 L), water (I L), and conc. HCl (92.6 mL, 1.11 mol). This was followed by the addition of Fe (218 g, 3.88 mol), in portions at 80° C. The resulting solution was stirred for 1 h at 80° C. The reaction mixture was cooled to room temperature with a water/ice bath. The resulting solution was diluted with 1 L of ethyl acetate. The pH value of the solution was adjusted to 9 with sat. NaHCO₃(aq.). The resulting solution was extracted with 1 L of ethyl acetate and the organic layers were combined and dried over anhydrous sodium sulfate. The dried solution was filtered and the filtrate was concentrated to afford a crude material containing 4-chloro-2-fluoro-5-methylaniline (Int-BBB1), which was used directly in the next step without purification.

### Step B: 2-bromo-4-chloro-6-fluoro-3-methylaniline (Int-BBB2)

Into a 3-L 3-necked round-bottom flask purged and maintained with an inert atmosphere of nitrogen were placed 4-chloro-2-fluoro-5-methylaniline (Int-BBB1) (178 g, 1.12 mol) and DMF (1.5 L). This was followed by the addition of NBS (198 g, 1.12 mol, 1.00 equiv), in portions at 0° C. The resulting solution was stirred for 1 h at room temperature. The reaction was then quenched by the addition of sat. NaHCO₃(aq.). The resulting solution was extracted with ethyl acetate (2×1 L) and the organic layers were combined. The organic layer was washed with water (2×1 L) and brine (1 L). The organic layer was dried over anhydrous sodium sulfate and concentrated. The residue was purified by silica gel column with ethyl acetate/petroleum ether (1:100) to afford 2-bromo-4-chloro-6-fluoro-3-methylaniline (Int-BBB2). MS (ESI): m z (M+H)⁺ 238, 240.

### Step C: 3-bromo-1-chloro-5-fluoro-4-iodo-2-methylbenzene (Int-BBB3)

Into a 5-L 4-necked round-bottom flask purged and maintained with an inert atmosphere of nitrogen were charged conc. H₂SO₄ (570 mL), water (2 L), and 2-bromo-4-chloro-6-fluoro-3-methylaniline (Int-BBB2) (190 g, 797 mmol). This was followed by the addition of a solution of NaNO₂ (60.5 g, 876 mmol) in H₂O (60 mL) while maintaining reaction temperature at 0-5° C. The resulting mixture was stirred for 30 min at 0-5° C. To this was added a solution of potassium iodide (529 g, 3.19 mol) in H₂O (500 mL) at 0-5° C. The resulting solution was stirred for 30 min at 0-5° C. The resulting solution was stirred for an additional 1 h at room temperature. The product mixture was then quenched by the addition of water. The resulting solution was extracted with ethyl acetate (2×200 mL) and the organic layers combined. The resulting mixture was washed with sat. Na₂S203 (500 mL) and brine (500 mL). The mixture was dried over anhydrous sodium sulfate. The dried solution was filtered and the filtrate was concentrated. The residue was purified by silica gel column, eluting with petroleum ether to afford 3-bromo-1-chloro-5-fluoro-4-iodo-2-methylbenzene (Int-BBB3). 1H NMR (500 MHz, CDCl₃) δ 7.25 (d, J=7.32 Hz, 1H), 2.70-2.80 (m, 3H).

### Step D: 2-bromo-4-chloro-6-fluoro-3-methylbenzaldehyde (Int-BBB4)

Into a 5-L 4-necked round-bottom flask purged and maintained with an inert atmosphere of nitrogen were charged 3-bromo-1-chloro-5-fluoro-4-iodo-2-methylbenzene (Int-BBB3) (131 g, 375 mmol) and THF (2.5 L). The resulting mixture was cooled to −78° C. This was followed by the addition of a 2.5M solution of n-BuLi in hexanes (151 mL, 376 mmol) dropwise with stirring at −78° C. The resulting mixture was stirred 30 min at −78° C. To this mixture was added DMF (30.2 g, 413 mmol) dropwise with stirring at −78° C. The resulting solution was stirred for 30 min at −78° C. The reaction was then quenched by the addition of 1 N HCl. The resulting solution was diluted with H₂O/ethyl acetate. The resulting solution was extracted with ethyl acetate (2-200 mL) and the organic layers were combined and dried over anhydrous sodium sulfate. The dried solution was filtered and the filtrate was concentrated. The residue was purified by silica gel column with ethyl acetate/petroleum ether (1:50) to afford 2-bromo-4-chloro-6-fluoro-3-methylbenzaldehyde (Int-BBB4).

### Step E: 4-bromo-6-chloro-5-methyl-1H-indazole (Int-BBB5)

Into a 3-L 3-necked round-bottom flask purged and maintained with an inert atmosphere of nitrogen were charged 2-bromo-4-chloro-6-fluoro-3-methylbenzaldehyde (Int-BBB4) (80.0 g, 318 mmol), DMSO (1.6 L) and hydrazine hydrate (191 g, 3.82 mol). The resulting solution was stirred for 3 h at 90° C. The reaction mixture was cooled to room temperature with a water/ice bath. The resulting solution was diluted with ice water. The resulting solution was extracted with ethyl acetate (2×250 mL) and the organic layers were combined. The organic layer was washed with water (2×500 mL) and brine (500 mL). The organic layer was dried over anhydrous sodium sulfate. The dried solution was filtered and the filtrate was concentrated. The residue was purified by silica gel column with ethyl acetate/hexane (1:8) to afford 4-bromo-6-chloro-5-methyl-1H-indazole (Int-BBB5). MS (ESI): m/z (M+H)⁺ 245.

### Step F: 4-bromo-6-chloro-5-methyl-1-(tetrahydro-2H-pyran-2-yl)-1H-indazole (Int-BBB6)

Into a 1-L 3-necked round-bottom flask purged and maintained with an inert atmosphere of nitrogen was placed 4-bromo-6-chloro-5-methyl-1H-indazole (Int-BBB5) (51.5 g, 210 mmol), THF (500 mL), PPTS (5.27 g, 21.0 mmol), and DHP (53.2 g, 632 mmol). The resulting solution was heated to reflux for 7 h. The product mixture was cooled with a water/ice bath. The resulting solution was diluted with ice water. The resulting solution was extracted with ethyl acetate (2×250 mL) and the organic layers were combined. The organic layer was washed with brine (500 mL). The mixture was dried over anhydrous sodium sulfate. The dried solution was filtered and the filtrate was concentrated. The residue was purified by silica gel column with ethyl acetate/petroleum ether (1:50) to afford 4-bromo-6-chloro-5-methyl-1-(tetrahydro-2H-pyran-2-yl)-1H-indazole (Int-BBB6). ¹H NMR (400 MHz, CDCl₃) δ 7.95 (d, J=1 Hz, 1H), 7.64 (d, J=1 Hz, 1H), 5.63 (dd, J=9, 3 Hz, 1H), 4.04-3.95 (m, 1H), 3.79-3.68 (m, 1H), 2.61-2.57 (m, 3H), 2.56-2.42 (m, 1H), 2.18-2.02 (m, 2H), 1.82-1.58 (m, 3H).

### Intermediate CCC6: 4-bromo-5,6-dimethyl-1-(tetrahydro-2H-yran-2-yl)-1H-indazole (Int-CCC6)

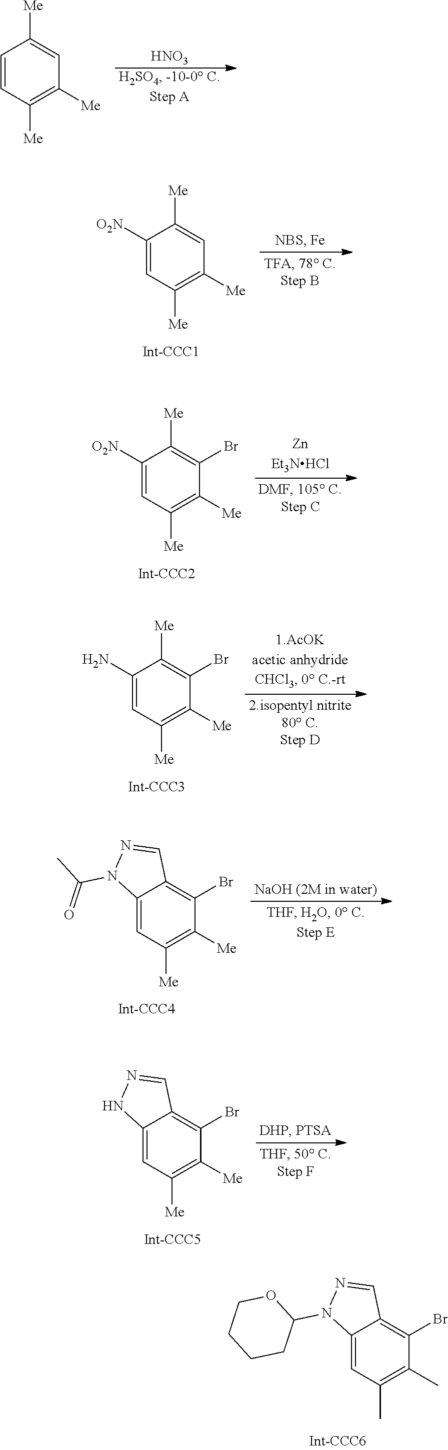

### Step A: 1.3,4-trimethyl-6-nitrobenzene (Int-CCC)

To a stirred solution of 1.2,4-trimethylbenzene (450 g, 3.74 mol) in H₂SO₄ (5.4 L) was added HNO₃ (236 g, 3.74 mol) dropwise at −10° C. under nitrogen atmosphere. The resulting mixture was stirred for additional 2 h at -10-0° C. The reaction was poured into a flask containing 20 L ice. The resulting solid was collected by vacuum filtration and washed with water (4 L). The resulting solid was dried under infrared light to afford 1.3,4-trimethyl-6-nitrobenzene (Int-CCC1) which was used in subsequent steps without further purification.

### Step B: 3-bromo-.2.4-trimethyl-5-nitrobenzene (Int-CCC2)

To a stirred solution of 1.3,4-Trimethyl-6-nitrobenzene (Int-CCC1) (396, g, 2.40 mol) and NBS (512, g, 2.88 mol) in TFA (4.75 L) was added Fe (4.00 g) in portions at room temperature. The resulting mixture was stirred for an additional 3 days at 78° C. The resulting mixture was concentrated under reduced pressure and diluted with ethyl acetate (5 L). The resulting mixture was washed with sat. NaHCO₃aq. (2×1 L). The organic layer was dried over anhydrous Na₂SO₄, filtered, and concentrated under reduced pressure. The residue was purified by silica gel column chromatography, eluted with PE/EtOAc (1:0-20:1) to afford 3-bromo-1,2,4-trimethyl-5-nitrobenzene (Int-CCC2).

### Step C: 3-bromo-2,4,5-trimethylaniline (Int-CCC3)

To a stirred solution of 3-bromo-1,2,4-trimethyl-5-nitrobenzene (Int-CCC2) (477 g, 1.95 mol) in DMF (5.8 L) were added Zn (575 g, 8.80 mol) and triethylamine HCl (1.47 kg, 10.7 mol) in portions at room temperature. The resulting mixture was stirred overnight at 105° C. The resulting mixture was filtered through CELITE and the filtrate was diluted with EtOAc (5 L). The organic layer was washed with water (3×3 L). The organic layer was dried over anhydrous Na₂SO₄. The dried solution was filtered and the filtrate was concentrated under reduced pressure. The residue was purified by silica gel column chromatography, eluted with Petroleum ether/EtOAc (1:0-5:1) to afford 3-bromo-2,4,5-trimethylaniline (Int-CCC3).

### Step D: 1-(4-bromo-5.6-dimethyl-1H-indazol-1-yl)ethan-1-one (Int-CCC4)

To a stirred mixture of 3-bromo-2,4,5-trimethylaniline (Int-CCC3) (250 g, 1.17 mol) and potassium acetate (138 g, 1.40 mol) in CHCl₃ (3.5 L) was added acetic anhydride (358 g, 3.50 mol) dropwise at 0° C. under nitrogen atmosphere. The resulting mixture was stirred for additional 1 h at room temperature. To the above mixture was added isopentyl nitrite (274 g, 2.34 mol) dropwise at 60° C. The resulting mixture was stirred overnight at 60° C. The mixture was allowed to cool down to room temperature and quenched by the addition of sat. NaHCO₃ (1 L). The organic layer was dried over anhydrous Na₂SO₄. The dried solution was filtered and the filtrate was concentrated under reduced pressure. The residue was purified by silica gel column chromatography, eluting with petroleum ether/EtOAc (1:0-5:2) to afford 1-(4-bromo-5.6-dimethyl-1H-indazol-1-yl)ethan-1-one (Int-CCC4).

### Step E: 4-bromo-5, 6-dimethyl-1H-indazole (Int-CCC5)

Into a 3 L 4-necked round-bottom flask were added 1-(4-bromo-5,6-dimethyl-1H-indazol-1-yl)ethan-1-one (134 g, 502 mmol), THF (700 mL) and H₂O (450 mL) at 0° C. To the stirred solution was added NaOH (756 mL, 2 M aqueous solution) dropwise at 0° C. and the resulting mixture was stirred for additional 2 h at 0° C. The reaction was diluted by the addition of EtOAc (1 L) at room temperature. The resulting mixture was extracted with EtOAc (3×1 L). The combined organic layers were dried over anhydrous Na₂SO₄. The dried solution was filtered and the filtrate was concentrated under reduced pressure. The residue was purified by silica gel column chromatography, eluting with petroleum ether/EtOAc (1:0-5:2) to afford 4-bromo-5, 6-dimethyl-1H-indazole (Int-CCC5).

### Step F: 4-bromo-5,6-dimethyl-1-(tetrahydro-2H-pyran-2-yl)-1H-indazole (Int-CCC6)

To a stirred solution of 4-bromo-5,6-dimethyl-1H-indazole (Int-CCC5) (97.0 g, 431 mmol) in THF (2 L) were added dihydropyran (72.5 g, 862 mmol) and p-toluenesulfonic acid (7.42 g, 43.1 mmol) in portions at room temperature and stirred overnight at 50° C. The mixture was allowed to cool down to room temperature. The reaction was quenched by the addition of sat. NaHCO₃(1 L) at room temperature. The resulting mixture was extracted with EtOAc (2×1 L). The combined organic layers were dried over anhydrous Na₂SO₄. The dried solution was filtered and the filtrate was concentrated under reduced pressure. The residue was purified by silica gel column chromatography, eluting with petroleum ether/EtOAc (40:1) to afford 4-bromo-5,6-dimethyl-1-(tetrahydro-2H-pyran-2-yl)-1H-indazole (Int-CCC6). ¹H NMR (300 MHz, DMSO-dr) δ 7.92 (s, 1H), 7.57 (s, 1H), 5.81-5.77 (m, 1H), 3.90-3.83 (m, 1H), 3.79-3.67 (m, 1H), 2.47-2.29 (m, 7H), 2.11-1.87 (m, 2H), 1.86-1.51 (m, 3H).

### Intermediate DDD4: 4-bromo-6-chloro-3,5-dimethyl-1-(tetrahydro-2H-pyran-2-yl)-1H-indazole (Int-DDD4)

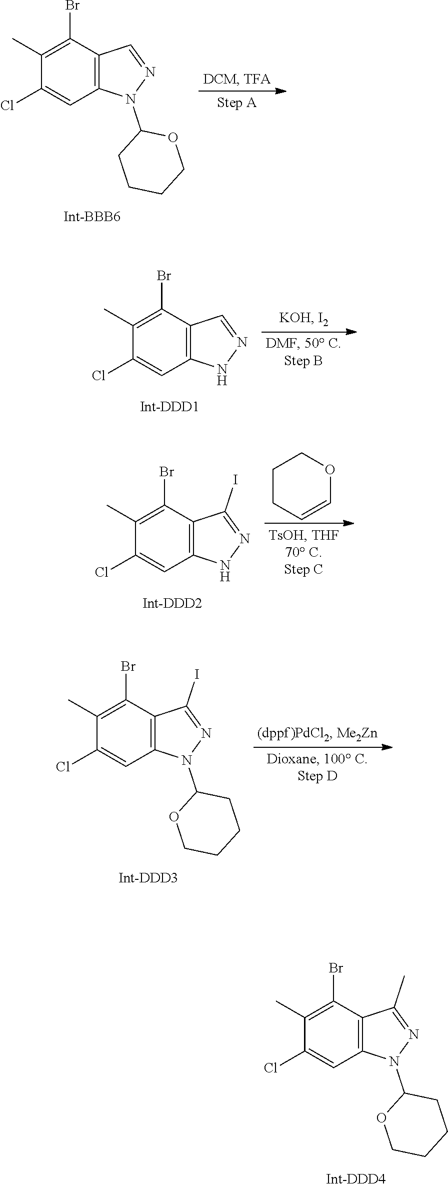

### Step A: 4-bromo-6-chloro-5-methyl-1H-indazole (Int-DDD1)

To a flask containing 4-bromo-6-chloro-5-methyl-1-(tetrahydro-2H-pyran-2-yl)-1H-indazole (Int-BBB6) (5.0 g, 15 mmol) and DCM (150 mL) was added trifluoroacetic acid (23 mL, 0.30 mol). The mixture was allowed to stir overnight at room temperature. The mixture was then concentrated under reduced pressure. The resulting mixture was diluted with ethyl acetate and saturated aqueous sodium bicarbonate. The organic layer was separated, dried over magnesium sulfate, filtered and concentrated under reduced pressure. The resulting mixture was purified by column chromatography on silica gel (0-30% ethyl acetate gradient in hexanes) to afford 4-bromo-6-chloro-5-methyl-1H-indazole (Int-DDD1). MS (ESI) m/z 245, 247 [M+H]⁺.

### Step B: 4-bromo-6-chloro-3-iodo-5-methyl-1H-indazole (Int-DDD2)

To a flask containing 4-bromo-6-chloro-5-methyl-1H-indazole (Int-DDD1) (2.8 g, 11 mmol) and DMF (38 mL) were added potassium hydroxide (1.9 g, 34 mmol) and then iodine (5.7 g, 23 mmol). The mixture was heated to 50° C. overnight. After stirring overnight, the mixture was allowed to cool to room temperature and then diluted with ethyl acetate and water. The organic layer was separated, dried over magnesium sulfate and filtered. Silica gel (30 grams) was added to the flask and the mixture was then concentrated under reduced pressure. The mixture was placed under vacuum for one hour and then purified by silica gel column chromatography (0-40% ethyl acetate gradient in hexanes) to afford 4-bromo-6-chloro-3-iodo-5-methyl-1H-indazole (Int-DDD2). MS (ESI) m: 371, 373 [M+H]⁺.

### Step C: 4-bromo-6-chloro-3-iodo-5-methyl-1-(tetrahydro-2H-pyran-2-yl)-1H-indazole (Int-DDD3)

To a flask containing 4-bromo-6-chloro-3-iodo-5-methyl-1H-indazole (Int-DDD2) (3.1 g, 8.4 mmol) and THF (56 mL) were added 3.4-dihydro-2H-pyran (1.5 mL, 17 mmol) and then p-toluenesulfonic acid monohydrate (0.16 g, 0.84 mmol). The mixture was heated to 70° C. overnight. After heating overnight, the mixture was allowed to cool to room temperature. The mixture was then diluted with ethyl acetate and saturated aqueous sodium bicarbonate. The organic layer was separated, washed with brine, dried over magnesium sulfate, filtered and concentrated under reduced pressure. The resulting mixture was purified by column chromatography on silica gel (0-5% ethyl acetate gradient in hexanes) to afford 4-bromo-6-chloro-3-iodo-5-methyl-1-(tetrahydro-2H-pyran-2-yl)-1H-indazole (Int-DDD3). MS (ESI) m/z 455, 457 [M+H]⁺.

### Step D: 4-bromo-6-chloro-3,5-dimethyl-1-(tetrahydro-2H-pyran-2-yl)-1H-indazole (Int-DDD4)

To a flask containing 4-bromo-6-chloro-3-iodo-5-methyl-1-(tetrahydro-2H-pyran-2-yl)-1H-indazole (Int-DDD3) (0.60 g, 1.3 mmol) was added dioxane (26 mL). [1.1′-bis(diphenylphosphino)ferrocene]dichloropalladium (0.15 g, 0.20 mmol) was added and argon was bubbled through the mixture for 5 minutes. Dimethylzinc (2.0 M in toluene, 0.66 mL, 1.3 mmol) was added slowly and the mixture was heated to 100° C. for 30 min. The mixture was allowed to cool to room temperature and was then slowly quenched with methanol (2.0 mL) and aqueous HCl (1.0 N, 4.0 mL). The mixture was allowed to stir at room temperature for 20 min. Ethyl acetate and saturated aqueous ammonium chloride were added. The mixture was filtered through CELITE. The organic layer was separated, washed with brine, dried over magnesium sulfate, filtered and concentrated under reduced pressure. The resulting mixture was purified by column chromatography on silica gel (0-10% ethyl acetate gradient in hexanes) to afford 4-bromo-6-chloro-3,5-dimethyl-1-(tetrahydro-2H-pyran-2-yl)-1H-indazole (Int-DD4). MS (EST) m/z 343, 345 [M+H]⁺. ¹H NMR (500 MHz, DMSO-d₆) δ 7.94 (s, 1H), 5.78 (dd, J=10, 2 Hz. 1H). 3.89-3.83 (m, 1H), 3.78-3.69 (m, 1H), 2.66 (s, 3H), 2.52 (s, 3H), 2.38-2.27 (m, 1H), 2.05-1.97 (m, 1H), 1.95-1.86 (m, 1H), 1.77-1.64 (m, 1H), 1.59-1.52 (m, 2H).

Intermediates in the table below were prepared via the same route for Int-DDD4, starting from Int-CCC6 and Int-AAA5 respectively.

| | | | [M + H]⁺ |
|---|---|---|---|
| Int | Structure | Compound Name | Found |
| Int-EEE4 | 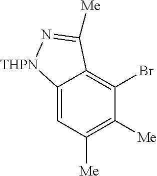 | 4-bromo-3,5,6-trimethyl-1- (tetrahydro-2H-pyran-2-yl)-1H- indazole | 323, 325 |
| Int-FFF4 | 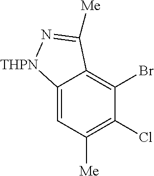 | 4-bromo-5-chloro-3,6-dimethyl-1- (tetrahydro-2H-pyran-2-yl)-1H- indazole | 343, 345 |

### Intermediate GGG2: 4-bromo-3,6-dichloro-5-methyl-1-(tetrahydro-2H-pyran-2-yl)-1H-indazole (Int-GGG-2)

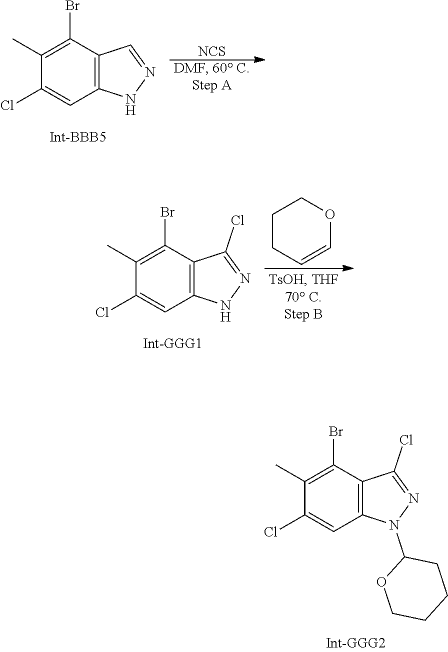

### Step A: 4-bromo-3,6-dichloro-5-methyl-1H-indazole (Int-GGG1)

To a flask containing 4-bromo-6-chloro-5-methyl-1H-indazole (Int-BBB5) (460 mg, 1.87 mmol) and DMF (10 mL) was added N-chlorosuccinimide (275 mg, 2.06 mmol). The mixture was heated to 60° C. for 18 hours. After 18 hours, the mixture was allowed to cool to room temperature and then diluted with ethyl acetate and water. The organic layer was separated, dried over magnesium sulfate, filtered and then concentrated under reduced pressure. The resulting residue was purified by silica gel column chromatgraphy (0-20% ethyl acetate gradient in hexanes) to afford 4-bromo-3,6-dichloro-5-methyl-1H-indazole (Int-GGG1). MS (ESI) m-: 279, 281 [M+H]⁺

### Step B: 4-bromo-3,6-dichloro-5-methyl-14tetrahydro-2H-pyran-2-yl)- 1H-indazole (Int-GGG2)

To a flask containing 4-bromo-3,6-dichloro-5-methyl-1H-indazole (Int-GGG1) (0.49 g, 1.8 mmol) and THF (12 mL) was added 3.4-dihydro-2H-pyran (0.32 mL. 3.5 mmol) and then p-toluenesulfonic acid monohydrate (33 mg, 0.18 mmol). The mixture was heated to 70° C. for 18 hours. After 18 hours, the mixture was allowed to cool to room temperature and then diluted with ethyl acetate and saturated aqueous sodium bicarbonate. The organic layer was separated, dried over magnesium sulfate, filtered and then concentrated under reduced pressure. The resulting residue was purified by silica gel column chromatography (0-5% ethyl acetate gradient in hexanes) to afford 4-bromo-3,6-dichloro-5-methyl-1-(tetrahydro-2H-pyran-2-yl)-1H-indazole (Int-GGG2). MS (ESI) m/z 363, 365 [M+H]⁺.

### Intermediate HHH5: 4-bromo-6-methyl-1-(tetrahydro-2H-pyran-2-yl)-5-(trifluoromethyl)-1H-indazole (Int-HHH5)

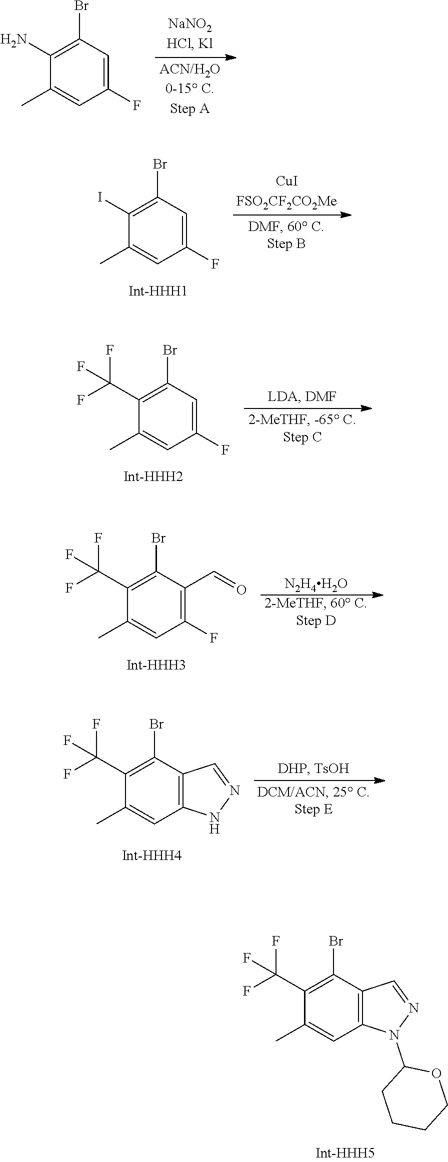

### Step A: 1-bromo-5-fluoro-2-iodo-3-methylbenzene (Int-HHH1)

2-Bromo-4-fluoro-6-methylaniline (Int-AAA1) (200 g, 0.983 mol) was dissolved in MeCN (800 mL). The resulting mixture was cooled down to 0° C. Concentrated HCl (12 M, 245 mL) was added into the reaction mixture while maintaining the reaction temperature at 0° C. A solution of NaNO₂ (81.1 g, 1.18 mol eq) in water (400 mL) was added dropwise into the reaction mixture maintaining the reaction temperature at 0° C. The resulting mixture was stirred for 0.5 h at 0° C. Then a solution of K1 (195 g, 1.18 mol) in water (400 mL) was added dropwise into the reaction mixture at 0° C. The resulting mixture was warmed up to room temperature and stirred for 12 h at 20° C. This reaction was repeated in one additional batch using the above conditions. The two batches of reactions were combined. The product mixture was adjusted to pH 8-9 by aq. NaOH and the aqueous phase was extracted with EtOAc (2.00 L×2). The organic phase was dried over Na₂SO₄, filtered, and concentrated. The residue obtained was purified by column chromatography (SiO₂, Petroleum ether: Ethyl acetate=1: 0 to 0: 1) to afford 1-bromo-5-fluoro-2-iodo-3-methylbenzene (Int-HHH1). ¹H NMR (400 MHz, CDCl₃) δ 7.27-7.22 (m, 1H), 6.95 (dd, J=2.4, 8.8 Hz, 1H). 2.56 (s, 3H).

### Step B: 1-bromo-5-fluoro-3-methyl-2-(trifluoromethyl)benzene (Int-HHH2)

1-Bromo-5-fluoro-2-iodo-3-methylbenzene (Int-HHH1) (100 g, 0.317 mol) was dissolved in DMF (1.50 L). To this mixture were added Cul (514 g, 2.70 mol) and methyl 2,2-difluoro-2-(fluorosulfonyl)acetate (518 g, 2.70 mol) at 25° C. The reaction mixture was heated and stirred for 12 h at 60° C. This reaction was repeated in 3 additional batches using the above conditions. The four batches of reactions were combined and quenched with water (24 L). The mixture was extracted with petroleum ether (8.00 L×2). The combined organic layers were washed with brine (4 L×2) and dried over Na₂SO₄. The dried solution was filtered and the filtrate was concentrated in vacuo to afford the crude material containing 1-bromo-5-fluoro-3-methyl-2-(trifluoromethyl)benzene (Int-HHH2), which was used directly into the next step without purification.

### Step C: 2-bromo-6-fluoro-4-methyl-3-(trifluoromethyl)benzaldehyde (Int-HHH3)

Bromo-5-fluoro-3-methyl-2-(trifluoromethyl)benzene (Int-HHH2) (100 g, 0.382 mol) was dissolved in 2-MeTHF (500 mL). The reaction mixture was cooled down to −65° C. A 2 M solution of LDA (213 mL, 426 mmol) was added into the mixture at −65° C. The reaction mixture was stirred for 0.5 h at -65° C. To this mixture was added dropwise DMF (31.2 g, 0.420 mol) at −65° C. The reaction mixture was stirred for 2 h at −65° C. This reaction was repeated in 2 additional batches using the above conditions. The three batches of reactions were combined. The reaction mixture pH was adjusted to 3-4 by using 1 M HCl and the aqueous phase was extracted with 2-MeTHF (500 mL×2). The organic phase was dried over Na₂SO₄, filtered, and concentrated to obtain 2-bromo-6-fluoro-4-methyl-3-(trifluoromethyl)benzaldehyde (Int-HHH3), which was used in the next step without further purification.

### Step D: 4-bromo-6-methyl-5-(trifluoromethyl)-1H-indazole (Int-HHH4)

2-Bromo-6-fluoro-4-methyl-3-(trifluoromethyl)benzaldehyde (Int-HHH3) (100 g, 0.351 mol) was dissolved in THF (800 mL). To this mixture was added N2H₄H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered and the filtrate was concentrated in vacuo to give the residue. This reaction was repeated in 2 additional batches using the above conditions. The three batches of reactions were combined. The residue obtained was triturated with DCM (100 mL) at 15° C. for 2 h. The solid was collected by filtration to afford 4-bromo-6-methyl-5-(trifluoromethyl)-1H-indazole (Int-HHH4). ¹H NMR (400 MHz, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step E: 4-bromo-6-methyl-1-(tetrahydro-2H-pyran-2-yl)-5-(trifluoromethyl)-1H-indazole (Int-HHH5)

2-Bromo-6-methyl-5-(trifluoromethyl)-1H-indazole (Int-HHH4) (60.0 g, 0.215 mol) was dissolved in DCM (240 mL) and MeCN (240 mL). DHP (21.7 g, 0.258 mol) and TsOH·H₂O (8.18 g, 0.043 mol) were added to the mixture at 20° C. The reaction mixture was stirred for 12 h at 20° C. Water (200 mL) was added to the product mixture. The resulting mixture was extracted with DCM (200 mL 2). The combined organic layers were washed with brine (200 mL) and dried over Na₂SO₄. The dried solution was filtered and the filtrate was concentrated under reduced pressure. The residue obtained was purified by column chromatography (SiO₂, Petroleum ether: Ethyl acetate=1:0 to 0:1) to afford 4-bromo-6-methyl-1-(tetrahydro-2H-pyran-2-yl)-5-(trifluoromethyl)-1H-indazole (Int-HHH5). ¹H NMR (400 MHz, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### Intermediate III4: 1-bromo-8-chloro-3-(methoxymethoxy)naphthalene (Int-III4

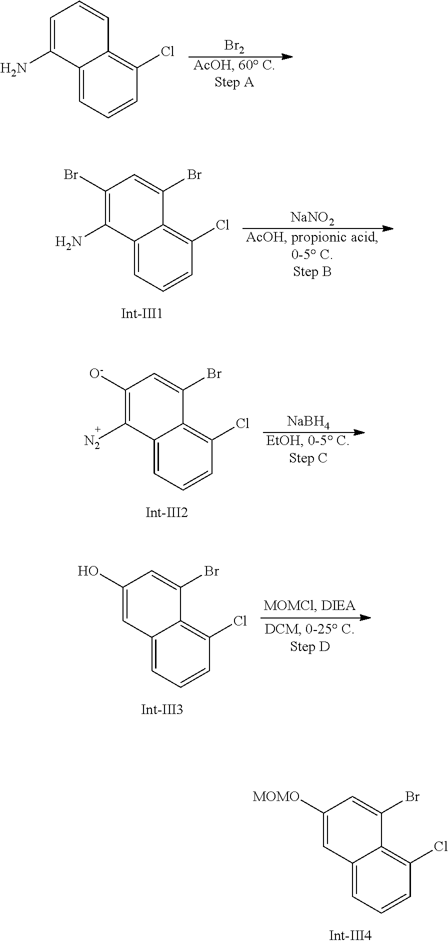

### Step A: 2,4-dibromo-5-chloronaphthalen-1-amine (Int-11H₁)

A solution of 5-chloronaphthalen-1-amine (300 g, 1.69 mol) was dissolved in AcOH (390 mL) in a 2.0 L reactor. The resulting mixture was heated to 60° C. and bromine (809 g, 5.07 mol, 261 mL) was added. The resulting mixture was stirred at 60° C. for 0.5 h. Another batch of the same reaction was conducted and both batches were combined. The combined product mixture was filtered and the filter cake was washed with acetic acid (2.0 L) to afford 2,4-dibromo-5-chloronaphthalen-1-anine (Int-II1). ¹H NMR (400 MHz, CDCl₃) δ 7.95 (s, 1H), 7.77 (dd, J=1.3, 8.5 Hz, 1H), 7.65 (dd, J=1.0, 7.5 Hz, 1H), 7.40-7.34 (m, 1H), 4.77-4.53 (m, 2H).

### Step B: Int-II12

A solution of 2,4-dibromo-5-chloronaphthalen-1-amine (Int-1 μl, 1.13 kg, 3.37 mol) in AcOH (12 L) was added into a 50 L reactor. Propionic acid (2.4 L) was added into the reaction vessel. The resulting mixture was cooled down to 0° C. To this mixture was added NaNO₂ (232 g, 3.37 mol) while maintaining the temperature between 0 and 5° C. The resulting mixture was stirred at 5° C. for 0.5 h. Water (10 L) was added into the product mixture. The resulting mixture was filtered and the filter cake was washed by water (5.0 L). The solid collected (Int-II12) was used directly in the next step without further purification or characterization.

### Step C: 4-bromo-5-chloronaphthalen-2-ol (Int-III3)

A solution of Int-III2 (370 g, 1.31 mol) in EtOH (2.0 L) was added into a 5.0 L reactor. The resulting mixture was cooled down to 0° C. To this mixture was added NaBH₄ (74.0 g, 1.96 mol) while maintaining the temperature between 0 and 5° C. The resulting mixture was warmed up to 25° C. and stirred for 16 h. The resulting mixture was cooled back down to ° C. And NaBH₄ (49.3 g, 1.31 mol) was added while maintaining the temperature between 0 and 5° C. The resulting mixture was warmed up to 25° C. and stirred for 16 h. The resulting mixture was cooled back down to ° C. And NaBH4 (74.0 g, 1.96 mol, 1.5 eq) was added while maintaining the temperature between 0 and 5° C. The resulting mixture was warmed up to 25° C. and stirred for 4 h. Another two batches of the same reaction were conducted and all three batches were combined. A 1.0 M aqueous solution of HCl (500 mL) was added into the combined mixtures. The resulting mixture was extracted with DCM (1.5 L). The organic layer was concentrated under reduced pressure. The crude product was purified by column chromatography (Eluent of 2-50% EtOAc/Pet, ether) to give 4-bromo-5-chloronaphthalen-2-ol (Int-II13). ¹H NMR (400 MHz, CDCl₃) δ 7.65-7.58 (m, 2H), 7.50 (dd, J=7, 1 Hz, 1H), 7.31 (tJ=8 Hz., 1H), 7.17 (d, J=3 Hz, 1H).

### Step D: 1-bromo-8-chloro-3-(methoxymethox inaphthalene (Int-III4)

A solution of 4-bromo-5-chloronaphthalen-2-ol (Int-1113, 90.0 g, 349 mmol) in DCM (900 mL) was added into a 2.0 L reactor. The resulting mixture was cooled down to 0° C. and DIEA (135 g, 1.05 mol, 182 mL) was added into the reaction mixture. To this mixture was added MOMCl (68.1 g, 845 mmol, 64.2 mL) dropwise into the reaction mixture while maintaining the temperature between 0 and 5° C. The reaction mixture was allowed to warm up to 25° C. and stirred for 16 h. Another two batches of the same reaction were conducted and all three batches were combined. Water (3.0 L) was added into the product mixture. The resulting mixture was extracted with DCM (500 mL). The organic layer was concentrated under reduced pressure. The crude product was purified by column chromatography (Eluent of 5-25% EtOAc/Pet, ether) to give 1-bromo-8-chloro-3-(methoxymethoxy)naphthalene (Int-III4). ¹H NMR (400 MHz, CDCl₃) δ 7.80 (dd, J=1, 8 Hz. 1H), 7.72 (d, J=2 Hz, 1H), 7.56 (dd, J=1, 8 Hz, 1H), 7.49 (d, J=2 Hz, 1H), 7.42-7.36 (m, 1H), 5.29 (s, 2H), 3.46 (s, 3H).

### Intermediate JJJ-1: 6-methyl-1-(tetrahydro-2H-pyran-2-yl)-5-(trifluoromethyl)-1H-indazol-4 crdhyd (Int-JJ-11

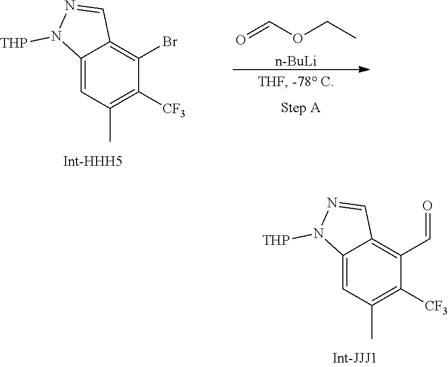

To a solution of 4-bromo-6-methyl-1-(tetrahydro-2H-pyran-2-yl)-5-(trifluoromethyl)-1H-indazole (Int-HHH5) (1 g, 2.75 mmol) in THF (15 mL) was added nBuLi (1,322 mL, 3.30 mmol, 2.5 M) at −78° C. under N₂ for 15 min. Ethyl formate (0.408 g, 5.51 mmol) was added to the mixture at −78° C. under Nz. The mixture was stirred at −78° C. for 0.5 h. The mixture was concentrated and purified by reverse phase HPLC using MeCN/water with 0.2% formic acid modifier to provide 6-methyl-1-(tetrahydro-2H-pyran-2-yl)-5-(trifluoromethyl)-1H-indazole-4-carbaldehyde (Int-JJJ-1). MS (ESI) [M+H]⁺ m/z 313.

### Intermediate LLL6: 8-bromo-N,N-bis(2,4-dimethoxybenzyl)-5.7

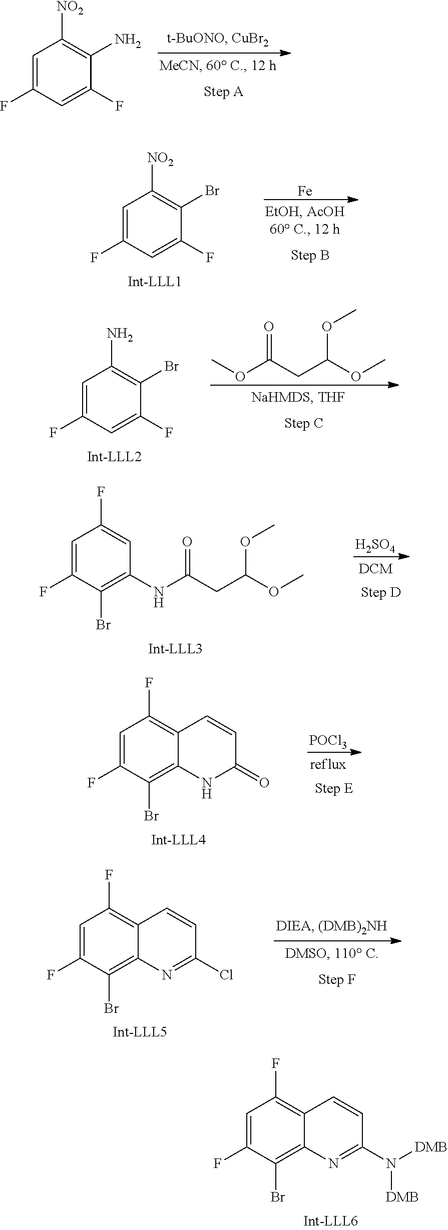

### Step A: 2-bromo-1.5-difluoro-3-nitrobenzene (Int-LLL1)

To a solution of tert-butyl nitrite (22.21 g, 215 mmol) and copper(II) bromide (38.5 g, 172 mmol) in MeCN (100 mL) was added a solution of 2,4-difluoro-6-nitroaniline (25 g, 144 mmol) in MeCN (1(0) mL) at 0° C. under N₂, and the mixture was stirred at 60° C. for 16 h. The solvent was evaporated under reduced pressure, and the residue was diluted with water (100 mL) and EtOAc (100 mL) and filtered. The filtrate was separated, and the organic layer was extracted with EtOAc (80 mL×3). The combined organic layer was washed with brine (2×100 mL), dried over Na₂SO₄, filtered and concentrated in vacuo to give the crude which was purified by flash Silica gel chromatography (Eluent of Pet, ether) to give 2-bromo-1,5-difluoro-3-nitrobenzene (Int-LLL1). ¹H NMR (400 MHz, CDCl₃) δ 7.51-7.44 (m, 1H), 7.18 (dt, J=3, 8 Hz, 1H). Step B; 2-bromo-3,5-difluoroaniline (Int-LLL2)

To a solution of 2-bromo-1,5-difluoro-3-nitrobenzene (Int-LLL1) (58 g, 244 mmol) in EtOH (250 mL) and AcOH (125 mL) was added iron powder (68.0 g, 1220 mmol). The reaction was stirred at 60° C. for 6 h. The reaction mixture was filtered and the filtrate was concentrated under reduced pressure to give crude material which was purified by flash silica gel chromatography (Eluent of 0-16% EtOAc/Pet, ether) to give 2-bromo-3,5-difluoroaniline (Int-LLL2). ¹H NMR (400 MHz, CDCl₃) δ 6.36-6.25 (m, 2H), 4.37 (br s. 2H).

### Step C; N-(2-bromo-3,5-difluorophenyl)-3,3-dimethoxy propanamide (Int-LLL3)

To a solution of 2-bromo-3,5-difluoroaniline (Int-LLL2) (5 g, 24.04 mmol) in THF (50 mL) were added methyl 3.3-dimethoxypropanoate (4.27 g, 28.8 mmol) and NaHMDS (36.1 mL, 36.1 mmol) (1M in THF) at 0° C. The reaction mixture was stirred at 25° C. for 2 h under N2 atmosphere. The reaction mixture was quenched with saturated aqueous NH₄Cl (40 mL), extracted with EtOAc (3×30 mL). The combined organic layer was washed with saturated aqueous NaCl (2×30 mL), dried over Na₂SO₄, filtered and the solvent was evaporated under reduced pressure to give the crude. The crude product was purified by flash silica gel chromatography (Eluent of 0-30% EtOAc/Pet, ether) to give N-(2-bromo-3,5-difluorophenyl)-3,3-dimethoxypropanamide (Int-LLL3).

¹H NMR (400 MHz, CDCl₃) δ 9.10 (br s, 1H), 8.16 (dd, J=2, 11 Hz, 1H), 6.80-6.58 (m, 1H), 4.74 (t. J=5 Hz. 1H), 3.48 (s, 6H), 2.81 (d, J=5 Hz, 2H).

### Step D: 8-bromo-5.7-difluoroquinolin-2(1H)-one (Int-LLL4)

To a stirred solution of N-(2-bromo-3,5-difluorophenyl)-3,3-dimethoxypropanamide (Int-LLL3) (7.2 g, 22 mmol) in DCM (80 mL) was added concentrated H₂SO₄ (17.76 mL, 333 mmol) at 0° C., and the mixture was stirred at 25° C. for 2 h. The reaction mixture was added into ice-water (30 mL), extracted with EtOAc (3×20 mL). The organic layer was washed with saturated brine (2×30 mL), dried over Na₂SO₄, filtered and the solvent was evaporated under reduced pressure to give the 8-bromo-5.7-difluoroquinolin-2(1H)-one (Int-LLL4). MS (ESI) [M+H] m/z: 260.

### Step E: 8-bromo-2-chloro-5.7-difluoroquinoline (Int-LLL5)

A mixture of 8-bromo-5,7-difluoroquinolin-2(1H)-one (Int-LLL4) (5 g, 19.23 mmol) in POCl₃(80 mL) was stirred at 110° C. for 3 h. The solvent was evaporated under reduced pressure to give crude material which was purified by flash silica gel chromatography (Eluent of 0-8% EtOAc/Petroleum ether) to give the 8-bromo-2-chloro-5,7-difluoroquinoline (Int-LLL5). MS (ESI) [M+H]⁺ m/z: 278, 280.

### Step F: 8-bromo-N,N-bis(2,4-dimethoxybenzyl)-5.7-difluoroquinolin-2-amine (Int-LLL6)

To a solution of 8-bromo-2-chloro-5,7-difluoroquinoline (Int-LLL5) (0.6 g, 2.155 mmol) in DMSO (5 mL) were added bis(2,4-dimethoxybenzyl)amine (0.821 g, 2.59 mmol) and N,N-diisopropylethylamine (1.13 mL, 6.46 mmol) at 25° C. The reaction was stirred at 120° C. for 3 h. The reaction mixture was quenched with H₂O (20 mL) and extracted with EtOAc (15 mL×3), dried over MgSO₄, filtered and concentrated to give crude material which was purified by flash silica gel chromatography (Eluent of 0-10% EtOAc/Pet, ether gradient) to give 8-bromo-N,N-bis(2,4-dimethoxybenzyl)-5,7-difluoroquinolin-2-amine (Int-LLL6). MS (ESI) [M+H]m-,z: 559.

### Intermediate MMM3: 2-(3-(methoxymethoxy)naphthalen-1-yl)-4,4,5,5-tetramethyl-1,3,2-dioxaborolane (Int-MMM3)

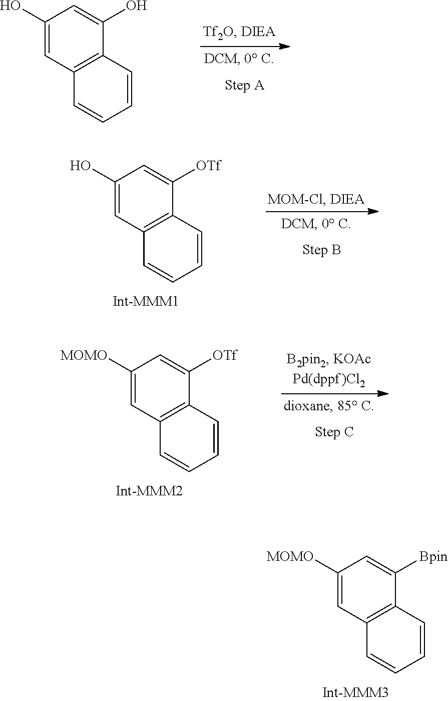

### Step A: 3-hydroxynaphthalen-1-yl trifluoromethanesulfonate (Int-MMM1)

A solution of naphthalene-1,3-diol (6.40g, 40.0 mmol) in DCM (200 mL) was added into a 3-necked flask that was equipped with two addition funnels separately charged with DIEA (6.98 mL, 40.0 mmol) and trifluoromethanesulfonic anhydride (6.72 mL, 40.0 mmol). The reaction vessel was cooled down to 0° C. The two reagents were added slowly dropwise at the same rate into the reaction. After completion of addition, the reaction was stirred at 0° C. for 2 h. The product mixture was diluted with DCM and washed with water twice and brine twice. The organic layer was concentrated down to dryness. The residue obtained was purified by silica gel column (0 to 15% ethyl acetate-hexane) to give 3-hydroxynaphthalen-1-yl trifluoromethanesulfonate (Int-MMM1). ¹H NMR (600 MHz, DMSO-d₆) δ 11.10 (s, 1H), 8.17 (d, J=8 Hz, 1H), 7.96 (d, J=8 Hz, 1H), 7.62 (t, J=8 Hz, 1H), 7.56 (dd, J=8, 7 Hz, 1H), 7.52 (d, J=2 Hz, 1H), 6.84 (d, J=2 Hz, 1H).

### Step B: 3-(methoxymethoxy)naphthalen-1-yl trifluoromethanesulfonate (Int-MMM2)

To a DCM (60 mL) solution of 3-hydroxynaphthalen-1-yl trifluoromethanesulfonate (Int-MMM1) (2.14 g, 7.32 mmol) and DIEA (1.92 mL, 11.0 mmol) was added chloromethyl methyl ether (0.612 mL, 8.06 mmol) at 0° C. The reaction was stirred at 0° C. for 1 h. Water was added to quench the reaction. The product mixture was extracted with more DCM. The organic layer was dried over anhydrous sodium sulfate and the dried solution was filtered. The filtrate was concentrated to dryness. The residue obtained was purified by silica gel column (0 to 15% ethyl acetate-hexane) to give 3-(methoxymethoxy)naphthalen-1-yl trifluoromethanesulfonate (Int-MMM2). ¹H NMR (600 MHz, DMSO-d6) δ 8.00 (d., =8 Hz, 1H), 7.88 (d, J=8 Hz, 1H), 7.71-7.59 (m, 3H), 7.44 (d, J=2 Hz, 1H), 5.38 (s, 2H), 3.44 (s, 3H).

### Step C: 2-(3-(methoxymethoxy)naphthalen-1-yl)-4,4,5,5-tetramethyl-1,3,2-dioxaborolane (Int-MMM3)

To a solution of 3-(methoxymethoxy)naphthalen-1-yl trifluoromethanesulfonate (Int-MMM2) (1.61 g, 4.79 mmol) and bis(pinacolato)diboron (2.44 g, 9.62 mmol) in 1.4-dioxane (24 mL) was added potassium acetate (1.41 g, 14.4 mmol) and [1,l-bis(diphenylphosphino)ferrocene] dichloropalladium(ii) (0.350 g, 0.479 mmol). The reaction mixture was heated at 85° C. for 3 h under N₂. The product mixture was diluted with water and extracted with ethyl acetate. The organic layer was dried over sodium sulfate. The dried solution was filtered and the filtrate was concentrated to dryness. The residue obtained was purified by silica gel column (0 to 10% ethyl acetate-hexane) to give 2-(3-(methoxymethoxy)naphthalen-1-yl)-4,4,5,5-tetramethyl-1,3,2-dioxaborolane (Int-MMM3). ¹H NMR (600 MHz, DMSO-d₆) δ 8.54 (d, J=8 Hz, 1H), 7.82 (d, J=8 Hz, 1H), 7.63 (d, J=3 Hz, 1H), 7.57 (d, J=3 Hz, 1H), 7.47 (ddd, J=8, 7, 1 Hz. 1H), 7.42 (ddd, J=8, 7, 1 Hz, 1H), 5.32 (s, 2H), 3.42 (s, 3H), 1.37 (s, 12H).

### Intermediate NNN2: potassium (8-chloro-3-(methoxymethoxy)naphthalen-1-yl)trifluoroborate (Int-NNN2)

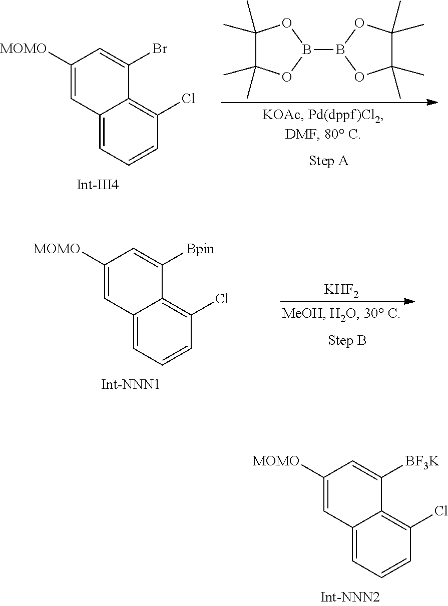

### Step A: 2-(8-chloro-3-(methoxymethoxy)naphthalen-1-yl)-4,4,5,5-tetramethyl-1,3,2-dioxaborolane (Int-NNN1)

To a solution of 1-bromo-8-chloro-3-(methoxymethoxy)naphthalene (Int-104) (200 mg, 0.663 mmol) in DMF (1 mL) were added 4,4,4′,4′,5,5,5′,5′-octamethyl-2,2′-bi(1,3,2-dioxaborolane) (168 mg, 0.663 mmol), potassium acetate (391 mg, 3.98 mmol) and [1.1′-bis(diphenylphosphino)ferrocene]dichloropalladium(II) (48.5 mg, 0.066 mmol) at 25° C. under N₂ atmosphere. Then the reaction mixture was stirred at 80° C. for 4 h and monitored by TLC that showed a new spot was formed. The reaction mixture was quenched with water (10 mL), extracted with EtOAc (3×10 mL). The combined organic layer was washed with saturated brine (3×10 mL), dried over Na₂SO₄, filtered and the solvent was evaporated under reduced pressure to give crude product. The crude product was purified by Prep-TLC (Pet, ether/EtOAc=5:1) to give 2-(8-chloro-3-(methoxymethoxy)naphthalen-1-yl)-4,4,5,5-tetramethyl-1,3,2-dioxaborolane (Int-NNN1). ¹H NMR (400 MHz, CDCl₃) δ 7.62 (d, J=8 Hz, 1H), 7.46-7.37 (m, 2H), 7.36-7.26 (m, 2H), 5.27 (s, 2H), 3.49 (s, 3H), 1.42 (s, 12H).

### Step B: potassium (8-chloro-34methoxymethoxy)naphthalen-1-yl)trifluoroborate (Int-NNN2)

To a solution of 2-(8-chloro-3-(methoxymethoxy)naphthalen-1-yl)-4,4,5,5-tetramethyl-1,3,2-dioxaborolane (Int-NNN1) (130 mg, 0.373 mmol) in MeOH (2.4 mL) was added a solution of potassium hydrogen fluoride (262 mg, 3.36 mmol) in H₂O (0.8 mL) at 25° C. Then the reaction was stirred at 30° C. for 3 h. The solvent was evaporated under reduced pressure to give potassium (8-chloro-3-(methoxymethoxy)naphthalen-1-yl)trifluoroborate (Int-NNN2) and was used in subsequent steps without further purification.

### Intermediate QQQ5: (5-hydroxy-2-((trifluoromethyl)thio)phenyl)boronic acid (Int-OOO5)

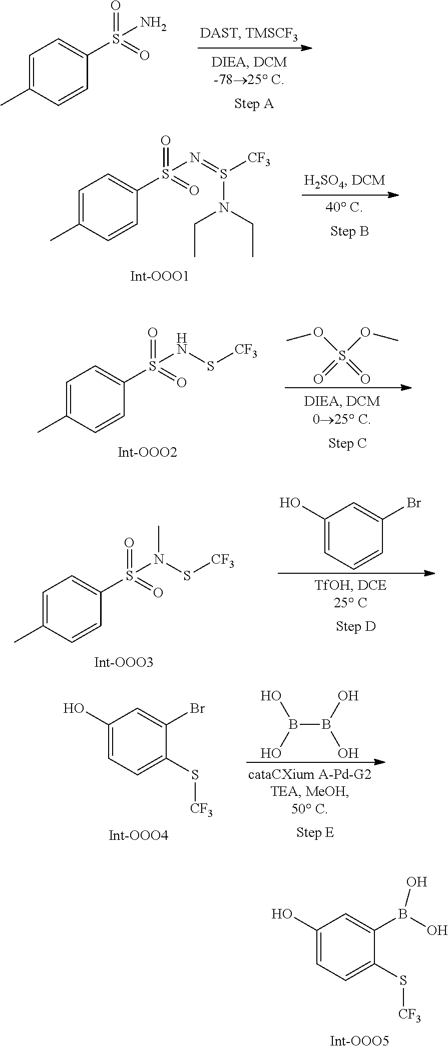

### Step A: (Z)-N-((diethylanuno)(trifluoromethyl)-λ⁴-sulfaneyldene)-4-methylbenzenesulfonamide (Int-OOO1)

To a stirred solution of DIEA (4.81 mL, 27.6 mmol) in anhydrous DCM (20 mL) cooled to −78° C. were added DAST (2.00 mL, 15.2 mmol), followed by the addition of (trifluoromethyl)trimethylsilane (1.96 g, 13.8 mmol) in 20 min interval. After one hour of stirring at −78° C., a solution of 4-methylbenzenesulfonamide (2.36 g, 13.8 mmol) in dry EtOAc (50 mL) was added dropwise to the above mixture at −78° C., and the reaction mixture was stirred at 25° C. 16 h. The reaction mixture was quenched with saturated NaHCO₃and extracted with EtOAc (30 mL×3), dried over anhydrous Na₂SO₄ and concentrated in vacuo to give the crude product. The crude product was purified by flash silica gel chromatography (0 to 27% EtOAc/Petroleum ether gradient) to give (Z)-N-((diethylamino)trifluoromethyl)-λ⁴-sulfaneyldene)-4-methylbenzenesulfonamide (Int-OOO1). MS (ESI) [M+H]⁺: m/z 343.

### Step B: 4-methyl-N-((trifluoromethylUthio)benzenesulfonamide (Int-OOO2)

To a stirred solution of (Z)-N-((diethylamino)(trifluoromethyl)- λ⁴-sulfaneyldene)-4-methylbenzenesulfonamide (Int-OOO1) (3.5 g, 10 mmol) in DCM (10 mL) was added concentrated H₂SO₄ (1.91 mL, 35.8 mmol) at 25° C. Then the reaction was stirred at 40° C. for 5 h. EtOAc (50 mL) was added into the reaction mixture, and the mixture was washed with distilled water (100 mL×4). The organic phase was dried over Na₂SO₄, filtered and concentrated in vacuo to give 4-methyl-N-((trifluoromethyl)thio) benzenesulfonanide (Int-OOO2). ¹H NMR (400 MHz, CDCl₃) δ 7.82 (d, J=8 Hz, 2H), 7.36 (d, J=8 Hz, 2H), 6.41 (br s. 1H), 2.46 (s, 3H).

### Step C: N,4-dimethyl-N-((trifluoromethyl)thio)benzenesulfonamide (Int-OOO3)

To a solution of 4-methyl-N-((trifluoromethyl)thio)benzenesulfonamide (Int-OOO2) (2.1 g, 7.7 mmol) in DCM (16 mL) were added DIEA (2.70 mL, 15.5 mmol) and dimethyl sulfate (0.99 mL, 10, mmol) at 0° C. under nitrogen atmosphere. The reaction mixture was warmed to 25° C. and stirred for 4 h. The reaction mixture was quenched with water (15 mL) and extracted with EtOAc (20 mL×3). The combined organic phase was dried over anhydrous Na₂SO₄, filtered and the filtrate was concentrated in vacuo to give a crude mixture. The crude mixture was purified by flash silica gel chromatography (eluent of 0 to 27% EtOAc/Petroleum ether gradient) to give N,4-dimethyl-N-((trifluoromethyl)thio)benzenesulfonamide (Int-OOO3). ¹H NMR (400 MHz, CDCl₃) δ 7.78 (d, J=8 Hz, 2H), 7.37 (d, J=8 Hz, 2H), 3.34 (s, 3H), 2.47 (s, 3H).

### Step D: 3-bromo-44(trifluoromethyl)thio)phenol (Int-OOO4)

To a solution of 3-bromophenol (0.75 g, 4.34 mmol) in dry DCE (10 mL) was added N,4-dimethyl-N-((trifluoromethyl)thio)benzenesulfonamide (Int-QQQ3) (1.484 g, 5.20 mmol) at 25° C. Then the reaction was stirred at 25° C. for 1 min. and then trifluoromethanesulfonic acid (0.077 mL, 0.87 mmol) was added slowly. The reaction mixture was stirred at 80° C. for 16 h. The reaction was diluted with DCM (10 mL), and then washed with water (3×10 mL) and saturated NaCl (3×10 mL). The organic phase was dried over Na₂SO₄, filtered and the filtrate was concentrated to give the crude mixture. The crude mixture was purified by reverse phase column chromatography (stationary phase: C18: mobile phase: 0 to 15% H₂O/MeCN gradient) to give 3-bromo-4-((trifluoromethyl)thio)phenol (Int-OOO4). ¹H NMR (400 MHz, CDCl₃) δ 7.65 (d, J=9 Hz, 1H), 7.24 (d, J=3 Hz, 1H), 6.84 (dd, J=3, 9 Hz. 1H).

### Step E: (5-hydroxy-2-((trifluoromethyl)thio)phenyl)boronic acid (Int-OOO5)

To a solution of 3-bromo-4-((trifluoromethyl)thio)phenol (Int-OOO4) (50 mg, 0.183 mmol) in MeOH (2 mL) were added hypodiboric acid (49.2 mg, 0.549 mmol), chloro[(di(1-adamantyl)-n-butylphosphine)-2-(2-aminobiphenyl)]palladium(II) (12.2 mg, 0.018 mmol) and TEA (0.077 mL, 0.55 mmol) at 25° C. The mixture was degassed and backfilled with N₂ (three times), and then heated at 50° C. and stirred for 1.5 h. The reaction mixture was concentrated in vacuo, and the residue was purified by preparative TLC (DCM/MeOH=10:1) to give (5-hydroxy-2-((trifluoromethyl)thio)phenyl)boronic acid (Int-OOO5). ¹H NMR (400 MHz, CDCl₃) δ 7.59 (d, J=8 Hz, 1H), 7.53 (d, J=3 Hz. 1H), 6.98 (dd, J=3, 8 Hz, 1H), 5.56 (br s, 2H).

### Intermediate PPP2: (5-hydroxy-3-methyl-2-((trifluoromethyl)thio)phenyl)boronic acid (Int-PPP2)

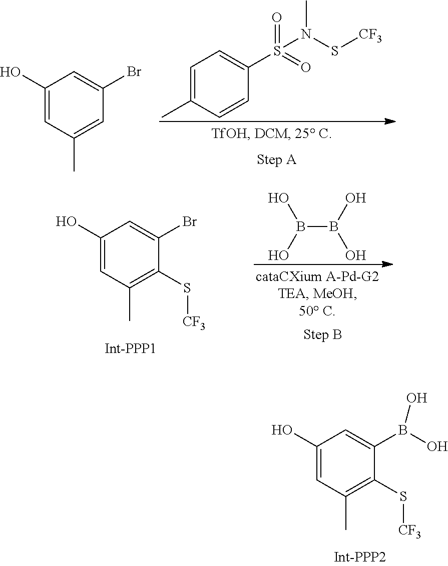

### Step A: 3-bromo-5-methyl-4-((trifluoromethyl)thio)phenol (Int-PPP)

To a solution of 3-bromo-5-methylphenol (200, mg, 1.07 mmol) in dry DCE (3 mL) was added N,4-dimethyl-N-((trifluoromethyl)thio)benzenesulfonamide (366 mg, 1.28 mmol) at 25° C. The reaction was stirred at 25° C. for 1 min, then trifluoromethanesulfonic acid (0.019 mL, 0.21 mmol) was added slowly, and the reaction mixture was stirred at 80° C. for 48 h. The reaction mixture was diluted with DCM (10 mL), and then washed with water (3×10 mL) and saturated NaCl (3×10 mL). The organic phase was dried over Na₂SO₄, filtered, and the filtrate was concentrated in vacuo to give the crude product. The crude product was purified by flash silica gel chromatography (Eluent of 0 to 15% EtOAc/Petroleum ether gradient) to give 3-bromo-5-methyl-4-((trifluoromethyl)thio)phenol (Int-PPP1). ¹H NMR (400 MHz, CDCl₃) δ 7.12 (d, J=3 Hz, 1H), 6.79 (d, J=2 Hz. 1H), 5.65 (br s, 1H), 2.58 (s, 3H).

### Step B: (5-hydroxy-3-methyl-2-((trifluoromethyl)thio)phenyl)boronic acid (Int-PPP2)

To a solution of 3-bromo-5-methyl-4-((trifluoromethyl)thio)phenol (Int-PPP1) (50 mg, 0.174 mmol) in MeOH (2 mL) were added hypodiboric acid (46.8 mg. 0.522 mmol), chlorol[di(1-adamantyl)-n-butylphosphine)-2-(2-aminobiphenyl)]palladium(II) (11.6 mg, 0.017 mmol) and TEA (0.073 mL, 0.522 mmol) at 25° C. The mixture was degassed and backfilled with N₂ (three times), and then stirred at 50° C. for 1.5 h to give a yellow mixture. The reaction was concentrated in vacuo, and the crude product was purified by preparative TLC (Pet, ether/EtOAc=1:1) to give (5-hydroxy-3-methyl-2-((trifluoromethyl)thio)phenyl)boronic acid (Int-PPP2). ¹H NMR (400 MHz, CDC) δ 7.36 (d, J=3 Hz, 1H), 6.94 (d, J=3 Hz, 1H), 5.54 (br s, 2H), 2.55 (s, 3H).

### Intermediate QQQ2: 4-(4,4,5,5-tetramethyl-1,3,2-dioxaborolan-2-yl)benzo[d]isothiazole (Int-OOO2)

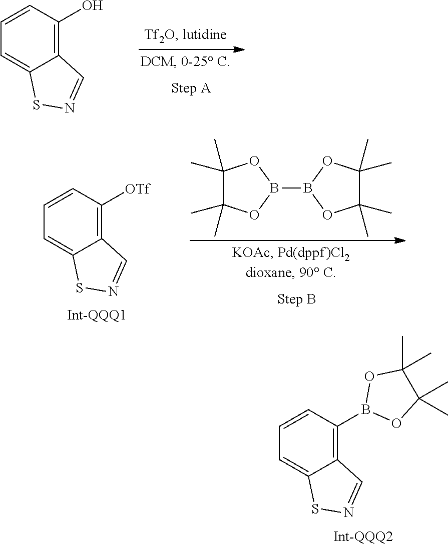

### Step A: benzo[d]isothiazol-4-yl trifluoromethanesulfonate (Int-OOO1)

To a solution of benzo[d]isothiazol-4-ol (100 mg, 0.661 mmol) and 2,6-dimethylpyridine (213 mg, 1.98 mmol) in DCM (1 mL) was added a solution of Tf₂O (0.15 mL, 0.86 mmol) in DCM (0.5 mL) at 0° C. under nitrogen. The reaction mixture was warmed to 25° C. and stirred at this temperature for 1 h. The reaction mixture was quenched with H₂O (2 mL) and DCM (2 mL), and separated. The aqueous layer was extracted with DCM (2 mL×3). The combined organic layers were dried over MgSO₄ and filtered. The filtrate was concentrated under reduced pressure to give the residue which was purified by Prep-TLC (SiO₂, Pet, ether/EtOAc=10:1, v/v) to give benzo[d]isothiazol-4-yl trifluoromethanesulfonate (Int-QQQ1). MS (ESI) [M+H]⁺ m/z: 283.

### Step B: 4(4,4,5,5-tetramethyl-1,3,2-dioxaborolan-2-yl)benzo[d]isothiazole (Int-O.0)

A mixture of benzo[d]isothiazol-4-yl trifluoromethanesulfonate (50.0 mg, 0.177 mmol). 4,4,4′,4′,5,5,5′,5′-octamethyl-2,2′-bi(1,3,2-dioxaborolane) (Int-QQQ1) (67.2 mg, 0.265 mmol), potassium acetate (52.0 mg, 0.530 mmol) and [1,1′-bis(diphenylphosphino)ferrocene]dichloropalladium (11) (12.9 mg, 0.018 mmol) in dioxane (1.5 mL) was sparged with nitrogen for 1 min. The reaction mixture was stirred at 100° C. for 12 h. The reaction mixture was diluted with EtOAc (5 mL), dried over MgSO₄ and filtered. The filtrate was concentrated under reduced pressure to give the residue which was purified by Prep-TLC (SiO₂, Pet, ether/EtOAc=10:1, v/v) to give 4-(4,4,5,5-tetramethyl-1,3,2-dioxaborolan-2-yl)benzo[d]isothiazole (Int-QQQ2). MS (ESI) [M+H]⁺ m/z: 262.

### Intermediate RRR4: (2-((tert-butoxycarbonyl)(methyl)amino)-7-fluorobenzold1thiazol-4-yl)boronic acid (Int-RRR4)

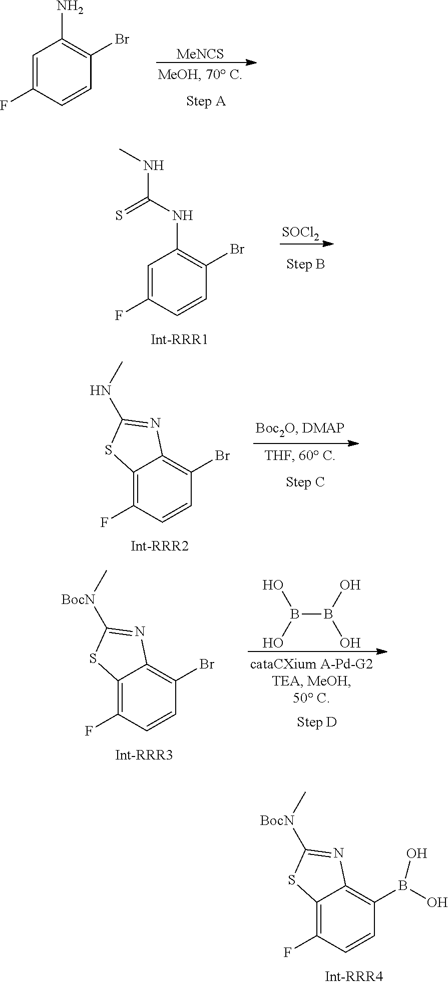

### Step A: 1-(2-bromo-5-fluorophenyl)-3-methylthiourea (Int-RRR1)

To a solution of 2-bromo-5-fluoroaniline (1 g, 5.26 mmol) in anhydrous MeOH (10 mL) was added methyl isothiocyanate (0.385 g, 5.26 mmol). The mixture was stirred at 70° C. for 5 h. The reaction mixture was concentrated under vacuum, and the crude product was purified by flash silica gel chromatography (eluent of 0-50% EtOAc/Pet, ether gradient) to give 1-(2-bromo-5-fluorophenyl)-3-methylthiourea (Int-RRR1). ¹H NMR (400 MHz, CDCl₃) δ 7.61 (dd, J=5.9, 8.6 Hz, 1H), 7.52 (br s, 11H), 6.89 (br d, J=1.2 Hz, 1H), 3.17 (d, J=4.7 Hz, 3H).

### Step B: 4-bromo-7-fluoro-N-methylbenzo[d]thiazol-2-amine (Int-RRR2)

1-(2-bromo-5-fluorophenyl)-3-methylthiourea (Int-RRR1) (200 mg, 0.760 mmol) was added into SOCl₂ (8 mL) at 20° C. After the addition was finished, the reaction mixture was stirred at 50° C. for 1 h. The solvent was removed under vacuum to give the crude mixture. The crude mixture was dissolved in DCM (10 mL), and the pH was adjusted with NaHCO₃(2 mL) to pH 8-9, and extracted with DCM (2×10 mL). The combined organic layers were dried over Na₂SO₄ and filtered. The filtrate was concentrated under reduced pressure to give the crude product 4-bromo-7-fluoro-N-methylbenzo[d]thiazol-2-amine (Int-RRR2). MS (EST) [M+H]⁺ m/z: 261, 263.

### Step C: tert-butyl (4-bromo-7-fluorobenzo[d]thiazol-2-ylvmethyl)carbamate (Int-RRR3)

To a stirred solution of 4-bromo-7-fluoro-N-methylbenzo[d]thiazol-2-amine (Int-RRR2) (212 mg, 0.812 mmol) in THF (6 mL) were added Boc-anhydride (0.566 mL, 2.436 mmol) and DMAP (9.92 mg, 0.081 mmol) at 20° C. After the addition was finished, the reaction was stirred at 60° C. for 3 h. The reaction mixture was concentrated under vacuum to give the crude product. The crude product was purified by flash silica gel chromatography (Eluent of 0-5% EtOAc/Pet, ether gradient) to give tert-butyl (4-bromo-7-fluorobenzo[d]thiazol-2-yl)(methyl)carbamate (Int-RRR3). MS (ESI) [M+H-tBu]⁺ m/z: 305, 307; [M+H-Boc]⁺ m/z: 261, 263.

### Step D: (2-((tert-butoxycarbonylmethyl)amino)-7-fluorobenzofdlthiazol-4-yl)boronic acid (Int-RRR4)

To a solution of tert-butyl (4-bromo-7-fluorobenzo[d]thiazol-2-yl)(methyl)carbamate (Int-RRR3) (120 mg, 0.332 mmol) in dioxane (1 mL) were added hypodiboric acid (119 mg, 1.329 mmol), triethylamine (0.139 mL, 0.997 mmol) and chloro[(di(1-adamantyl)-n-butylphosphine)-2-(2-aminobiphenyl)]palladium(II) (22.2 mg, 0.033 mmol) at 50° C. under N2 atmosphere. The mixture was stirred at 50° C. for 1 h. The mixture was quenched with water (5 mL), extracted with EtOAc (20 mL×3). The combined organic phase was dried over anhydrous Na₂SO₄, filtered and concentrated in vacuo. The residue was purified by preparative TLC plate (SiO₂. Pet, ether/EtOAc=10/1) to give (2-((tert-butoxycarbonyl)(methyl)amino)-7-fluorobenzo[d]thiazol-4-yl)boronic acid (Int-RRR4). MS (ESI) [M+H]⁺ m/z: 327.

### Intermediate SSS5: (2-((2.4-dimethoxybenzyl)amino)-5-fluoroquinolin-8-yl)boronic acid (Int-SSS5)

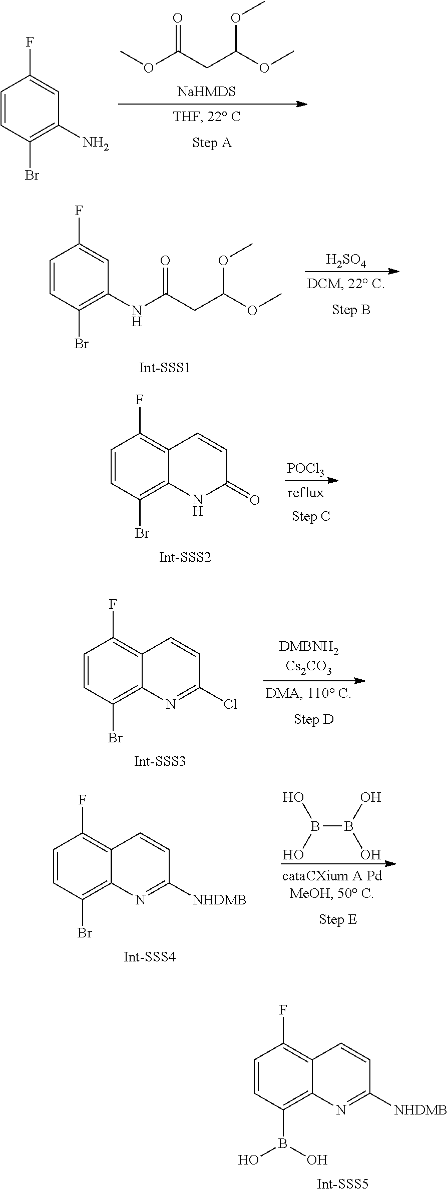

### Step A: N-(2-bromo-5-fluorophenyl)-3,3-dimethoxypropanamide (Int-SSS1)

NaHMDS in THF (19.7 mL, 19.7 mmol, IM) was added to a solution of 2-bromo-5-fluoroaniline (2500 mg. 13.16 mmol) and methyl 3.3-dimethoxypropanoate (2339 mg, 15.79 mmol) in THF (2.0 mL) at 0° C. The resulting solution was stirred at 22° C. for 16 h. The mixture was quenched with MeOH (1.0 mL). The solvent was removed under reduced pressure. The residue was purified by Prep-TLC (SiO₂, Pet, ether: EtOAc=5:1) to give N-(2-bromo-5-fluorophenyl)-3,3-dimethoxypropanamide (Int-SSS1). MS (ESI) [M+H]⁺ m/z: 307, 309.

### Step B: 8-bromo-5-fluoroquinolin-2(1H)-one (Int-SSS2)

To a solution of N-(2-bromo-5-fluorophenyl)-3,3-dimethoxypropanamide (Int-SSS1) (150 mg, 0.490 mmol) in CH₂Cl₂ (1 mL) was added conc. H₂SO₄ (0.392 mL, 7.35 mmol) at 0° C. The resulting solution was stirred at 22° C. for 16 h. The solvent was removed under reduced pressure. Ice water (5 mL) was added to the mixture and the solid was formed. The mixture was filtered and the filter cake was washed 3-fold with water and petroleum ether, and the solid was dried to give 8-bromo-5-fluoroquinolin-2(1H)-one (Int-SSS2). MS (ESI) [M+H]⁺ m z: 242, 244.

### Step C: 8-bromo-2-chloro-5-fluoroquinoline (Int-SSS3)

A solution of 8-bromo-5-fluoroquinolin-2(1H)-one (Int-SSS2) (110 mg, 0.454 mmol) in phosphoryl trichloride (3.00 g, 19.57 mmol) was stirred at 90° C. for 2 h. After the reaction was finished, the mixture was added into water (5 mL) slowly, and neutralized with 2 N NaOH aqueous to pH -7, the mixture was extracted with EtOAc (5 mL×3). The organic layers were washed with saturated NaCl, dried over Na₂SO₄, filtered and concentrated to give crude 8-bromo-2-chloro-5-fluoroquinoline (Int-SSS3). MS (ESI) [M+H]⁺ m/z 260, 262.

### Step D: 8-bromo-N-(2,4-dimethoxybenzyl)-5-fluoroquinolin-2-anine (Int-SSS4)

To a solution of 8-bromo-2-chloro-5-fluoroquinoline (Int-SSS3) (80 mg, 0.305 mmol) in DMA (2 mL) were added (2,5-dimethoxyphenyl)methanamine (153 mg, 0.914 mmol) and Cs₂CO₃ (496 mg, 1.524 mmol), the mixture was stirred at 110° C. for 15 h. The reaction was quenched with water (5 mL) and extracted with EtOAc (5 mL×3). The organic layers were washed with saturated NaCl (10 mL×3), dried over Na₂SO₄, filtered and concentrated. The residue was purified by prep-TLC (SiO₂, Pet, ether: EtOAc=3:1) to give 8-bromo-N-(2,4-dimethoxybenzyl)-5-fluoroquinolin-2-amine (Int-SSS4). MS (ESI) [M+H]⁺ m/z: 391, 393.

### Step E: (2-((2,4-dimethoxybenzyl)amino)-5-fluoroquinolin-8-yl)boronic acid (Int-SSS5)

To a solution of 8-bromo-N-(2,4-dimethoxybenzyl)-5-fluoroquinolin-2-amine (Int-SSS4) (80 mg, 0.20 mmol) in MeOH (2 mL) were added hypodiboric acid (55.0 mg, 0.613 mmol), chloro[(di(1-adamantyl)-n-butylphosphine)-2-(2-aminobiphenyl)]palladium(II) (13.67 mg, 0.020 mmol) and triethylamine (62.1 mg, 0.613 mmol), the mixture was stirred at 50° C. for 2 h under nitrogen. The mixture was concentrated, the residue was purified by prep-TLC (SiO₂, Pet, ether: EtOAc=2: 1) to give (2-((2,4-dimethoxybenzyl)amino)-5-fluoroquinolin-8-yl)boronic acid (Int-SSS5). MS (ESI) [M+H]¹′ m/z: 357.

### Intermediate TTT3: (2-((tert-butoxycarbonyl)amino)benzo[d]oxazol-4-yl)boronic acid (Int-TTT3)

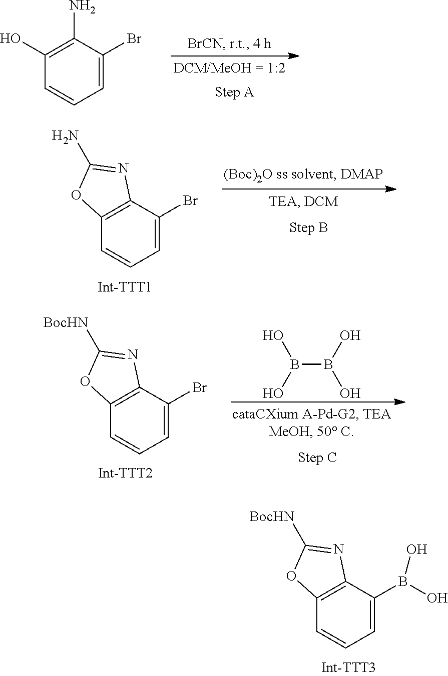

### Step A: 4-bromobenzo[d]oxazol-2-amine (Int-TTT1)

To a stirred solution of 2-amino-3-bromophenol (380 mg, 2.02 mmol) in CH₂Cl₂ (2.0 mL) and MeOH (4.0 mL) was added cyanogen bromide (420 mg, 3.97 mmol) at 20° C. The solution was stirred at 20° C. for 12 h. The mixture was purified by prep-TLC (SiO₂, Pet, ether/EtOAc=2:1) to give 4-bromobenzo[d]oxazol-2-amine (Int-TTT1). MS (ESI) [M+H]⁺ m/z: 213, 215.

### Step B: tert-butyl (4-bromobenzo[d]oxazol-2-yl)carbamate (Int-TTT2)

To a solution of 4-bromobenzo[d]oxazol-2-amine (Int-TTT1) (300 mg, 1.41 mmol) in di-tert-butyl dicarbonate (5.0 mL, 1.408 mmol) were added DMAP (86 mg, 0.70 mmol) and TEA (0.589 mL, 4.22 mmol) at 25° C. under N2 atmosphere. The mixture was stirred at 60° C. for 3 h. The mixture was cooled, and the solvent was evaporated under reduced pressure to give the crude product. The crude product was purified by flash silica gel chromatography (eluent of 0 to 15% EtOAc/Pet, ether gradient) to give tert-butyl (4-bromobenzo[d]oxazol-2-yl)carbamate (Int-TTT2). ¹H NMR (400 MHz, CDCl₃) δ 8.36 (br. s, 1H), 7.43 (dd, J=4.8, 8.0 Hz. 2H). 7.09 (t, J=8.0 Hz, 1H), 1.49-1.54 (n. 9H).

### Step C: (2-((tert-butoxycarbonyl)amino)benzo[d]oxazol-4-yl)boronic acid (Int-TTT3)

To a solution of tert-butyl (4-bromobenzo[d]oxazol-2-yl)carbamate (Int-TTT2) (160 mg, 0.511 mmol) in MeOH (4.0 mL) were added hypodiboric acid (137 mg, 1.53 mmol), TEA (0.214 mL, 1.53 mmol) and chloro[(di(1-adamantyl)-n-butylphosphine)-2-(2-aminobiphenyl)]palladium(II) (34.2 mg, 0.051 mmol) at 50° C. under a nitrogen atmosphere. The mixture was stirred at 50° C. for 2 h. The mixture was purified by preparative TLC (SiO₂. DCM: MeOH=20: 1) to give (2-((tert-butoxycarbonyl)amino)benzo[d]oxazol-4-yl)boronic acid (Int-TTT3). MS (ESI) [M+H]⁺ m/z: 279.

### Intermediate UUU4: (7-fluorobenzo[d]thiazol-4-yl)boronic acid (Int-UUU4)

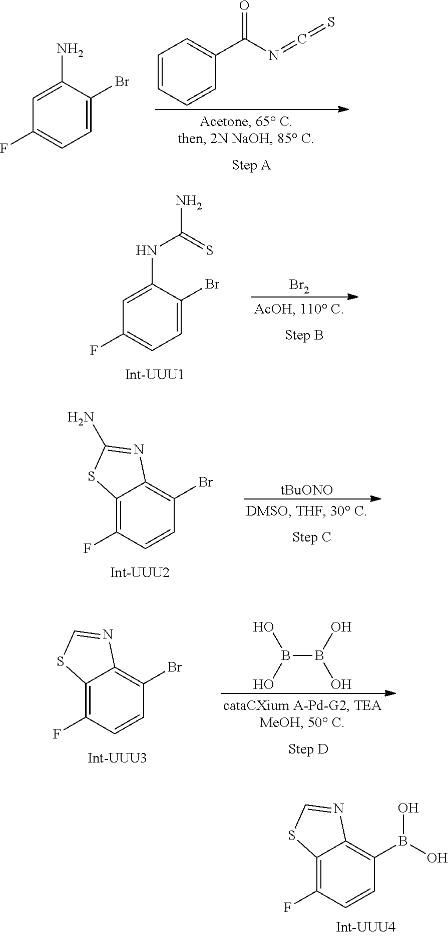

### Step A: 1-(2-bromo-5-fluorophenyl)thiourea (Int-UUU1)

To a stirred solution of benzoyl isothiocyanate (3.55 mL, 26.3 mmol) in acetone (50 mL) was added dropwise 2-bromo-5-fluoroaniline (5.00 g, 26.3 mmol), and the mixture was warmed to 65° C. and stirred for 30 min. The solvent was evaporated under reduced pressure to give the crude product. The crude product was dissolved in aqueous NaOH (50 mL, 2 N) at room temperature, and the mixture was stirred at 85° C. for 1 h. The reaction mixture was acidified with cold aqueous hydrochloric acid (6M) to pH 2, and stirred for 2 min. Then the pH was adjusted with aqueous NH₄OH (37%) to pH 10 and a precipitate was formed. The solid was collected by filtration and dried in vacuo to give 1-(2-bromo-5-fluorophenyl)thiourea (Int-UUU1). MS (ESI) [M+H]⁺ m/z 249, 251.

### Step B: 4-bromo-7-fluorobenzo[d]thiazol-2-amine (Int-UUU2)

A solution of Br₂ (5.21 mL, 101 mmol) in acetic acid (10 mL) was added to a stirred solution of 1-(2-bromo-5-fluorophenyl)thiourea (Int-UUU1) (6.30 g, 25.3 mmol) in acetic acid (50 mL), and the mixture was stirred at 110° C. for 3 h. The reaction mixture was concentrated in vacuo, and the residue was partitioned between water (20 mL) and EtOAc (40 mL×3). The combined organic phase was dried over anhydrous Na₂SO₄, filtered and concentrated in vacuo. The residue was purified by flash silica gel chromatography (eluent Pet, ether/EtOAc=3/1) to give 4-bromo-7-fluorobenzo[d]thiazol-2-anine (Int-UUU2). MS (ESI) [M+H]⁺ m/z: 247.0, 249.0. ¹H NMR (400 MHz, CDCl₃) δ 7.45 (dd, J=5.0, 8.7 Hz, 1H), 6.79 (t, J=8.8 Hz, 1H), 5.96 (m, 2H).

### Step C: 4-bromo-7-fluorobenzo[d]thiazole (Int-UUU3)

A solution of 4-bromo-7-fluorobenzo[d]thiazol-2-amine (Int-UUU2) (500 mg, 2.02 mmol) in THF (3 mL) was added dropwise over 20 min to a solution of tert-butyl nitrite (0.361 mL, 3.04 mmol) and DMSO (0.014 mL, 0.202 mmol) in THF (7 mL) at 30° C. The mixture was stirred at 30° C. for 1 h. The reaction mixture was quenched with water (2 mL) and extracted with EtOAc (10 mL×3). The combined organic phase was dried over anhydrous Na₂SO₄, filtered and concentrated in vacuo. The residue was purified by preparative TLC plate (SiO₂, Pet, ether/EtOAc=5/1) to give 4-bromo-7-fluorobenzo[d]thiazole (Int-UUU3) MS (ESI) [M+H]⁺ m/z: 232, 234.

### Ste p D: (7-fluorobenzo[d]thiazol-4-yl)boronic acid (Int-UUU4)

To a solution of 4-bromo-7-fluorobenzo[d]thiazole (Int-UUU3) (150 mg, 0.646 mmol) in MeOH (1 mL) were added hypodiboric acid (232 mg, 2.59 mmol), triethylamine (0.270 mL, 1.939 mmol) and chloro[(di(1-adamantyl)-n-butylphosphine)-2-(2-aminobiphenyl)]palladium(II) (43.2 mg, 0.065 mmol) under nitrogen atmosphere. The mixture was stirred at 50° C. for 1 h. The reaction was quenched with water (3 mL) and the resulting mixture was extracted with EtOAc (10 mL×3). The combined organic phase was dried over anhydrous Na₂SO₄ filtered and concentrated in vacuo. The residue was purified by preparative TLC plate (SiO₂. Pet, ether/EtOAc=10/1) to give (7-fluorobenzo[d]thiazol-4-yl)boronic acid (Int-UUU4). MS (ESI) [M+H]⁺ m/z: 198.

### Intermediate VVV2: (2-((tert-butoxycarbonyl)amino)-7-fluorobenzo[d]thiazol-4-yl)boronic acid (Int-VVV2)

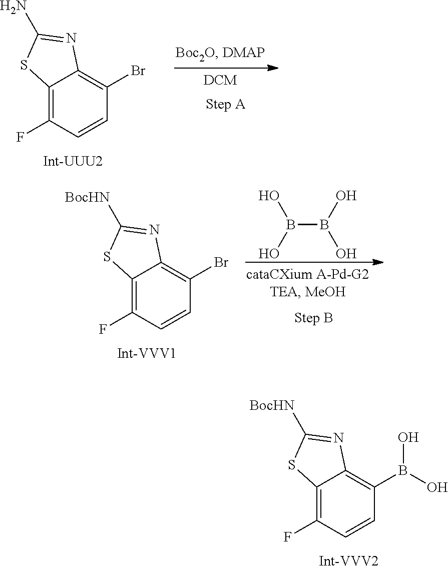 [SMILES: Nc1nc2c(Br)ccc(F)c2s1 ; CC(C)(C)OC(=O)Nc1nc2c(Br)ccc(F)c2s1 ; OB(O)B(O)O ; CC(C)(C)OC(=O)Nc1nc2c(B(O)O)ccc(F)c2s1]

### Step A: tert-butyl (4-bromo-7-fluorobenzo[d]thiazol-2-yl)carbamate (Int-VVV1)

In specific embodiments, the present disclosure provides a compound or a pharmaceutically acceptable salt thereof selected from the group consisting of: To a solution of 4-bromo-7-fluorobenzo[d]thiazol-2-amine (Int-UUU2) (95.0 mg, 0.384 mmol) in DCM (3 mL) were added TEA (0.161 mL, 1.15 mmol), DMAP (4.7 mg, 0.038 mmol) and Boc₂O (0.268 mL, 1.15 mmol) at 0° C. under a N₂ atmosphere. Then the mixture was stirred at 25° C. for 4 h. The residue was purified by prep-TLC (SiO₂, petroleum ether: ethyl acetate=3: 1) to give tert-butyl (4-bromo-7-fluorobenzo[d]thiazol-2-yl)carbamate (Int-VVV1). MS (ESI) [M+H-tBu]⁺ m/z: 291, 293.

### Step B: (2-((tert-butoxycarbonyl)amino)-7-fluorobenzo[d]thiazol-4-yl)boronic acid (Int-VVV2)

To a solution of tert-butyl (4-bromo-7-fluorobenzo[d]thiazol-2-yl)carbamate (Int-VVV1) (90 mg, 0.26 mmol) in MeOH (1 mL) were added hypodiboric acid (93 mg, 1.0 mmol), triethylamine (0.108 mL, 0.778 mmol) and chloro[(di(1-adamantyl)-n-butylphosphine)-2-(2-aminobiphenyl)]palladium(II) (17 mg, 0.026 mmol) under N₂ atmosphere. The mixture was stirred at 50° C. for 1 h. The mixture was evaporated under reduced pressure. The residue was purified by prep-TLC (SiO₂, petroleum ether: ethyl acetate=3:1) to give (2-((tert-butoxycarbonyl)amino)-7-fluorobenzo[d]thiazol-4-yl)boronic acid (Int-VVV2). MS (EST) [M+H]⁺ m/z: 313.

### Intermediate WWW4: 4-(4,4,5,5-tetramethyl-1,3,2-dioxaborolan-2-yl)-1,3-dihydro-2H-naphtho[2,3-d]imidazol-2-one (Int-WWW4)

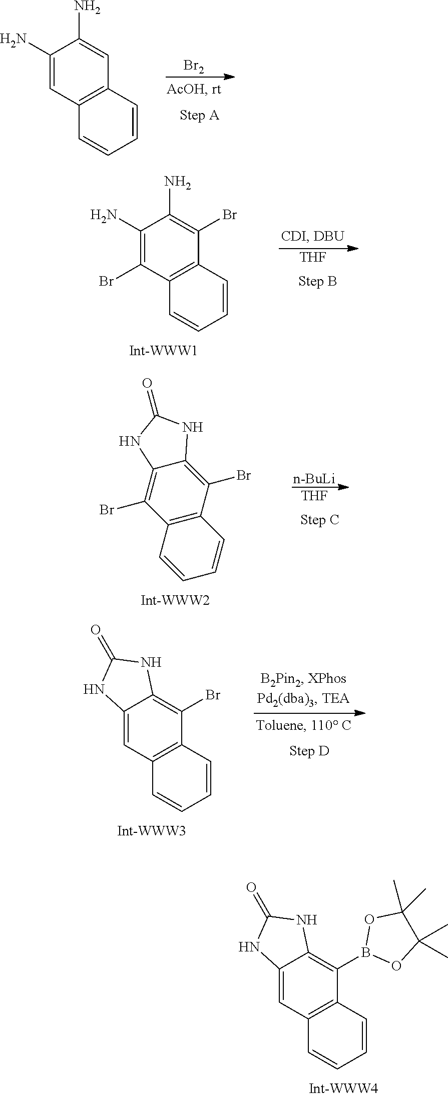

### Step A: 1.4-dibromonaphthalene-2,3-diamine (Int-WWW)

A mixture of bromine (1.62 mL, 31.6 mmol) in AcOH (30 mL) was added dropwise into a solution of naphthalene-2,3-diamine (2.00 g, 12.6 mmol) in AcOH (40 mL), and the mixture was stirred at 20° C. for 0.5 h. The mixture was filtered and the filter cake was washed with aqueous Na₂CO₃ (100 mL, 2 wt %), and then washed with water (100 mL). The solid was concentrated in vacuo to give 1.4-dibromonaphthalene-2,3-diamine (Int-WWW1). ¹H NMR (400 MHz, DMSO-d₆) δ 7.87-7.75 (m, 2H), 7.35-7.22 (m, 2H), 5.69 (s, 4H).

### Step B: 4.9-dibromo-1,3-dihydro-2H-naphtho[2,3-d]imidazol-2-one (Int-WWW2)

To a stirred solution of 1.4-dibromonaphthalene-2,3-diamine (Int-WWW1) (1.50 g, 4.75 mmol) in THF (10 mL) were added di(1H-imidazol-1-yl)methanone (1.92 g, 11.9 mmol) and DBU (1.45 g, 9.49 mmol) at 0° C., and the mixture was stirred at 40° C. for 16 h. The mixture was concentrated in vacuo, and the residue was diluted with water (50 mL) and DCM (50 mL). The mixture was filtered and the filtered cake was washed with water (50 mL) and DCM (50 mL). The solid was collected and dried under vacuum to afford 4,9-dibromo-1,3-dihydro-2H-naphtho[2,3-d]imidazol-2-one (Int-WWW2). MS (ESI): m/z (M+H)⁺ 349.

### Step C: 4-bromo-1,3-dihydro-2H-naphtho[2,3-d]imidazol-2-one (Int-WWW3)

n-BuLi (2.5 M in Hexane, 2.25 mL, 5.61 mmol) was added to a solution of 4,9-dibromo-1,3-dihydro-2H-naphthol[2,3-d]imidazol-2-one (Int-WWW2) (6W) mg, 1.754 mmol) in THF (20 mL) at −78° C. over 2 min. After stirring at -78° C. for 0.5 h, the reaction mixture was quenched with saturated NH₄Cl (5 mL). The solvent was removed under reduced pressure and the residue was diluted with 20 mL of EtOAc. The solid was collected by filtration and purified by reverse phase HPLC (MeCN/water with 0.1% TFA modifier) to give 4-bromo-1,3-dihydro-2H-naphtho[2,3-d]imidazol-2-one (Int-WWW3). MS (ESI): m/z (M+H)⁺ 263.

### Step D: 4-(4,4,5,5-tetramethyl-1,3,2-dioxaborolan-2-yl)-1,3-dihydro-2H-naphtho[2,3-d]imidazol-2-one (Int-WWW4)

A mixture of 4-bromo-1,3-dihydro-2H-naphtho[2,3-d]imidazol-2-one (Int-WWW3) (150 mg, 0.570 mmol), 4,4,4′,4′,5,5,5′,5′-octamethyl-2,2′-bi(1,3,2-dioxaborolane) (290 mg, 1.14 mmol), triethylamine (173 mg, 1.71 mmol), XPhos (40.8 mg. 0.086 mmol) and Pd₂(dba)₃ (39 mg, 0.043 mmol) in toluene (20 mL) was evacuated and backfilled with nitrogen (three times). The mixture was stirred at 110° C. for 17 h. The mixture was filtered and the filtrate was concentrated. The residue was purified by Prep-TLC (silica gel, ethyl acetate/Pet, ether=1/1, v/v) to give 4-(4,4,5,5-tetramethyl-1,3,2-dioxaborolan-2-yl)-1,3-dihydro-2H-naphtho[2,3-d]imidazol-2-one (Int-WWW4). MS (ESI): m/z (M+H)⁺ 311.

### Intermediate XXX3: (2-(bicyclo[1.1,1]pentan-1-yl)-5-(methoxymethoxy)phenyl)boronic acid (Int-XXX3)

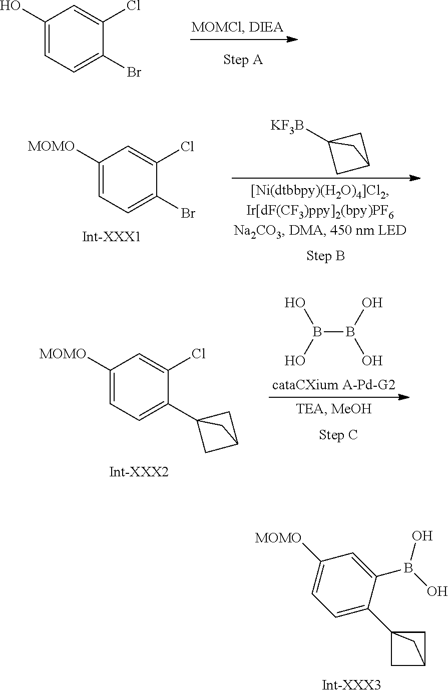

### Step A: 1-bromo-2-chloro-4-(methoxymethoxy) benzene (Int-XXX1)

To a solution of 4-bromo-3-chlorophenol (6.50 g, 31.3 mmol) and DIEA (16 mL, 94 mmol) in anhydrous DCM (18 mL) cooled to 0° C. was slowly added chloro(methoxy)methane (6.2 mL, 82 mmol). The solution was warmed to room temperature (20° C.) and stirred for 1 h. The mixture was evaporated under reduced pressure to give the crude product. The residue was purified by flash silica gel chromatography (eluent of 0-20% EtOAc/Pet, ether gradient) to give 1-bromo-2-chloro-4-(methoxymethoxy)benzene (Int-XXX1). ¹H NMR (400 MHz, CDCl₃) δ 7.48 (d, J=−9.2 Hz, 1H), 7.18 (d, J=2.8 Hz, 1H), 6.83 (m, J=2.8, 9.0 Hz, 1H), 5.14 (s, 2H), 3.47 (s, 3H).

### Step B: 1-(2-chloro-4-(methoxymethoxy)phenyl)bicyclo[1.1.1]pentane (Int-XXX2)

To a solution of 1-bromo-2-chloro-4-(methoxymethoxy)benzene (Int-XXX1) (50.0 mg, 0.199 mmol) in N,N-dimethylacetamide (1.5 mL) were added potassium bicyclo[1.1.1]pentan-1-yltrifluoroborate (51.9 mg, 0.298 mmol), [Ni(dtbbpy)(H₂O)₄]Cl₂ (14 mg, 0.030 mmol) and Ir[dF(CF₃)ppy]₂(dtbpy)PF₆ (22.30 mg, 0.020 mmol) and Na₂CO₃ (42.1 mg, 0.398 mmol) under N₂ atmosphere. The mixture was sealed and placed in a photoreactor (450 nm light source, 100% intensity, 1000 rpm stirring and 10000 rpm fan speed) for 4 h. The mixture was cooled, diluted with water (0 mL), extracted with ethyl acetate (2×2 mL), dried over Na₂SO., filtered and the solvent was evaporated under reduced pressure to give the crude product. The residue was purified by preparative TLC (SiO₂, petroleum ether) to give 1-(2-chloro-4-(methoxymethoxy)phenyl)bicyclo[1.1.1]pentane (Int-XXX2). ¹H NMR (500 MHz, CDCl₃) δ 7.27 (d, J=8.5 Hz, 1H), 7.21 (d, J=2.5 Hz, 1H), 7.07 (m, J=2.5, 8.5 Hz, 1H), 5.34 (s, 2H), 3.68-3.66 (m, 3H), 2.76 (s, 1H), 2.42 (s, 6H).

### Step C: (2(bicyclo[1.1.1]pentan-1-yl)-5-(methoxymethoxy)phenyl)boronic acid (Int-XXX3)

A mixture of 1-(2-chloro-4-(methoxymethoxy)phenyl)bicyclo[1.1.1]pentane (Int-XXX2) (23 mg, 0.096 mmol), hypodiboric acid (25.9 mg, 0.289 mmol), chloro[(di(1-adamantyl)-n-butylphosphine)-2-(2-aminobiphenyl)]palladium(II) (6.44 mg, 9.64 μmol) and TEA (0.040 mL, 0.289 mmol) in MeOH (1 mL) was evacuated and backfilled with nitrogen (three times). The mixture was heated at 50° C. for 0.5 h. The reaction was concentrated and the residue was purified by preparative TLC plate eluting with 30% EtOAc/Pet, ether to give (2-(bicyclo[1.1.1]pentan-1-yl)-5-(methoxymethoxy)phenyl)boronic acid (Int-XXX3). ¹H NMR (500 MHz, McOD) δ 7.14 (d, J=8.4 Hz, 1H), 6.97-6.93 (m, 1H), 6.86-6.83 (m, 1H), 5.16 (s, 2H), 3.45 (s, 3H), 2.50 (s, 1H), 2.07 (s, 6H).

### Intermediate YYY2: (5-(methoxymethoxy)-2-(trimethylsilyl)phenyl)boronic acid (Int-YYY2)

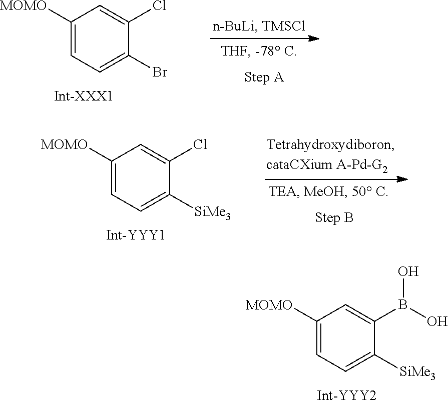

### Step A: (2-chloro-4-(methoxymethoxy)phenyl)trimethylsilane (Int-YYY1)

To a stirred solution of 1-bromo-2-chloro-4-(methoxymethoxy)benzene (Int-XXX1) (200 mg. 0.795 mmol) in THF (5 mL) was added n-butylthium (0.700 mL, 1.75 mmol) (2.5 M in hexane) at −78° C., and the mixture was stirred at −78° C. under N₂ atmosphere for 3 min. Then TMSCI (0.202 mL, 1.59 mmol) was added, and the resulting mixture was stirred at −78° C. for another 10 min. The reaction mixture was quenched with aqueous ammonium chloride (2 mL), and extracted with ethyl acetate (20 mL). The combined organic layers were washed with brine (5 mL), dried over Na₂SO₄, filtered and the solvent was evaporated under reduced pressure. The residue was purified by preparative TLC plate eluting with 5% EtOAc/Pet. Ether to give (2-chloro-4-(methoxymethoxy)phenyl)trimethylsilane (Int-YYY1). ¹H NMR (400 MHz, CDCl₃) δ 7.36 (d, J=8.2 Hz, 1H), 7.07 (d, J=2.0 Hz, 1H), 6.93 (dd, J=2.3, 8.6 Hz, 1H), 5.18 (s, 2H), 3.50-3.46 (m, 3H), 0.35 (s, 9H).

### Step B: (54methoxymethoxy)-2-(trimethylsilyl)phenyl)boronic acid (Int-YYY2)

A mixture of (2-chloro-4-(methoxymethoxy)phenyl)trimethylsilane (Int-YYY1) (300 mg, 0.858 mmol), hypodiboric acid (231 mg, 2.57 mmol), cataCXium A-Pd-G2 (57 mg, 0.086 mmol) and triethylamine (0.359 mL, 2.57 mmol) in MeOH (1 mL) was evacuated and backfilled with N₂ (three times). The mixture was heated and stirred at 50° C. for 0.5 h. The reaction mixture was concentrated in vacuo, and the residue was purified by preparative TLC plate eluting with 30% EtOAc/Pet, ether to give (5-(methoxymethoxy)-2-(trimethylsilyl)phenyl)boronic acid (Int-YYY2). ¹H NMR (500 MHz, CDCl₃) S 7.55 (d, J=8.2 Hz, 1H), 7.25 (d, J=2.4 Hz, 1H), 7.06 (dd, J 2.6, 8.2 Hz, 1H), 5.20 (s, 2H), 4.79 (s, 2H), 3.48 (s, 3H), 0.32 (s, 9H).

### Intermediate ZZZ6: terl-butyl 17-fluoro-4-(4,4,5,5-tetramethyl-1,3,2-dioxaborolan-2-yl)-1,3-benzothiazol-2-yl)carbamate (Int-ZZZ6)

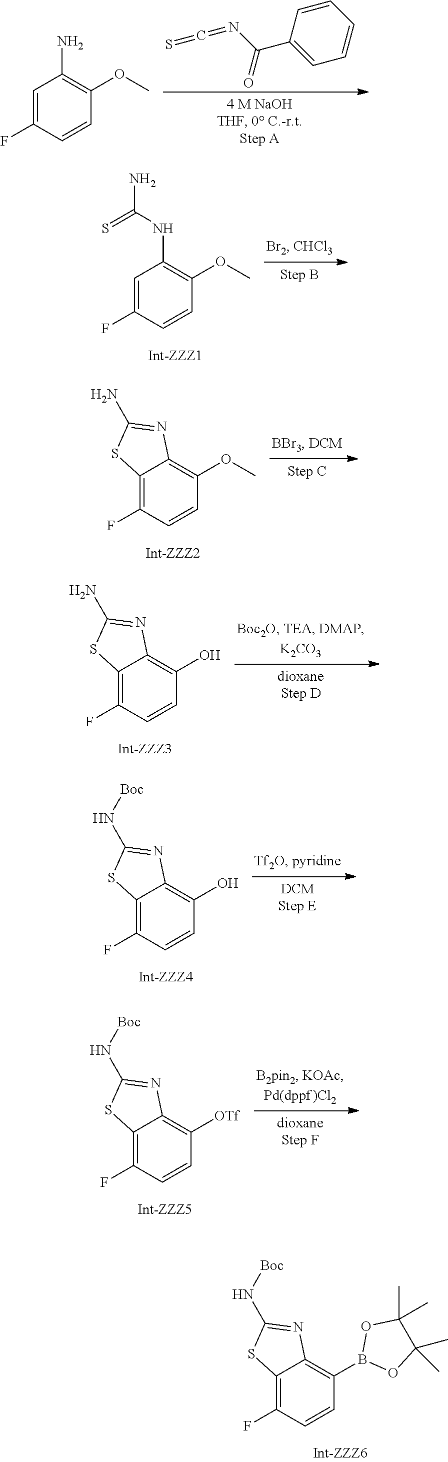

### Step A: 1-(5-fluoro-2-methoxyphenyl)thiourea (Int-ZZZ1)

A solution of benzoyl isothiocyanate (5.73 mL, 42.5 mmol) in THF (60 mL) was cooled to 0° C. under nitrogen, and then 5-fluoro-2-methoxyaniline (6.00 g, 42.5 mmol) was added dropwise maintaining the internal reaction temperature below 10° C. Then the mixture was warmed to 25° C. and stirred for 30 min. Then NaOH (4 M, 10.2 mL, 51.0 mmol) in water (12 mL) was added to the mixture, and the mixture was stirred at 85° C. for 5 h. The reaction mixture was filtered, and the solid was collected and washed with cold hexanes to give 1-(5-fluoro-2-methoxyphenyl)thiourea (Int-ZZZ1). MS (ESI): m z (M+H)⁺ 201.

### Step B: 7-fluoro-4-methoxybenzo[d]thiazol-2-amine (Int-ZZZ2)

To a solution of 1-(5-fluoro-2-methoxyphenyl)thiourea (Int-ZZZ1) (5.00 g, 25.0 mmol) in THF (50 mL) was added Br₂ (1.286 mL, 24.97 mmol) at 0° C. dropwise for 15 min under N₂ atmosphere. The mixture was stirred at 0° C. for 30 min. Then the mixture was stirred at 85° C. for 7 h, and a precipitate was formed. The precipitate was filtered and washed with cold DCM to give 7-fluoro-4-methoxybenzo[d]thiazol-2-amine (Int-ZZZ2). MS (ESI): m/z (M+H)⁺ 199.

### Step C: 2-amino-7-fluorobenzo[d]thiazol-4-ol (Int-ZZZ3)

A solution of BBr₃ (6.01 mL, 63.6 mmol) in DCM (45 mL) was cooled to 0° C. under N2 atmosphere. Then 7-fluoro-4-methoxybenzoldithiazol-2-amine (Int-ZZZ2) (4.20 g, 21.2 mmol) was added to the mixture for 15 min. The reaction mixture was slowly warmed to 25° C. and stirred for 18 h. The mixture was quenched carefully with MeOH at 0° C. then poured slowly into 5 M aqueous Na₂CO₃: the mixture was evaporated under reduced pressure to give the crude product 2-amino-7-fluorobenzo[d]thiazol-4-ol (Int-ZZZ3). MS (ESI): m/z (M+H)⁺ 185.

### Step D: tert-butyl (7-fluoro-4-hydroxy-1,3-benzothiazol-2-yl)carbamate (Int-ZZZ4)

To a solution of 2-amino-7-fluorobenzo[d]thiazol-4-ol (Int-ZZZ3) (4.00 g, 21.7 mmol) in dioxane (1 mL) were added triethylamine (9.08 mL, 65.2 mmol), DMAP (0.133 g, 1.09 mmol) and (Boc)₂₀ (11.6 mL, 49.9 mmol) at 25° C. under N₂ atmosphere. The mixture was stirred at 25° C. for 1 h. The mixture was diluted with water (15 mL), ethyl acetate (25 mL) and saturated aqueous NaCl (10 mL). The layers were separated and the organic layer was collected and concentrated in vacuo. The residue was treated with MeOH (20 mL) and K₂CO₃ (1.28 g, 9.23 mmol), and the mixture was stirred at 25° C. for 2 h. The mixture was evaporated under reduced pressure and the residue was diluted with water (3 mL) and extracted with ethyl acetate (5 mL). The organic layer was dried over Na₂SO₄, filtered and the solvent was evaporated under reduced pressure to give the crude product. The crude product was purified by flash silica gel chromatography (Eluent of 0 to 30% ethyl acetate/petroleum ether gradient) to give tert-butyl (7-fluoro-4-hydroxy-1,3-benzothiazol-2-yl)carbamate_(Int-ZZZ4). MS (ESI): m/z (M+H)⁺ 284.

### Step E: 2-[(tert-butoxcarbonyl)amino]-7-fluoro-1,3-benzothiazol-4-yl trifluoromethanesulfonate (Int-ZZZ)

To a solution of tert-butyl (7-fluoro-4-hydroxy-1,3-benzothiazol-2-yl)carbamate (Int-ZZZ4) (2.00 g, 7.06 mmol) in DCM (10 mL) were added pyridine (1.14 mL, 14.1 mmol) and Tf₂O (1.43 mL, 8.47 mmol) at 0° C. under nitrogen atmosphere. The mixture was stirred at 0° C. for 1 h. The mixture was cooled, diluted with saturated aqueous NaHCO₃(10 mL), and extracted with ethyl acetate (15 mL×3). The combined organic layer was dried over Na₂S4, filtered and the solvent was evaporated under reduced pressure to give the crude product. The crude product was purified by flash silica gel chromatography (eluent of 0-30% ethyl acetate/petroleum ether gradient) to give 2-[(tert-butoxycarbonyl)amino]-7-fluoro-1,3-benzothiazol-4-yl trifluoromethanesulfonate (Int-ZZZ5). MS (ESI): m/z (M-56+H)⁺ 361.

### Step F: tert-butyl [7-fluoro-4-(4,4,5,5-tetramethyl-1,3,2-dioxaborolan-2-yl)-1,3-benzothiazol-2-yl]carbamate (Int-ZZZ6)

To a solution of 2-[(tert-butoxycarbonyl)amino]-7-fluoro-1,3-benzothiazol-4-yl trifluoromethanesulfonate (Int-ZZZ5) (1.20 g, 2.89 mmol) in dioxane (40 mL) were added bis(pinacolato)diboron (5.87 g, 23.1 mmol), potassium acetate (0.851 g, 8.67 mmol) and PdCl₂(dppf) (0.211 g, 0.289 mmol) at 25° C. under a N2 atmosphere. The mixture was stirred at 100° C. for 16 h, cooled to rt, treated with water (30 mL) and extracted with EtOAc (30 mL×3). The combined organic layer was dried over Na₂SO₄, filtered and concentrated under vacuum to give the crude product. The crude product was purified by flash silica gel chromatography (eluent of 0-30% ethyl acetate/petroleum ether gradient) to give tert-butyl [7-fluoro-4-(4,4,5,5-tetramethyl-1,3,2-dioxaborolan-2-yl)-1,3-benzothiazol-2-yl]carbamate (Int-ZZZ6). ¹H NMR (4(X) MHz, CDCl₃) δ 7.77 (dd, J=6.4, 8.3 Hz, 1H), 6.93 (t, J=8.8 Hz, 1H), 1.52 (d, J=3.2 Hz, 9H), 1.35 (s, 12H).

### Intermediate AB1: 1-fluoro-4-(4,4,5,5-tetramethyl-1,3,2-dioxaborolan-2-yl)naphthalen-2-ol (Int-AB1)

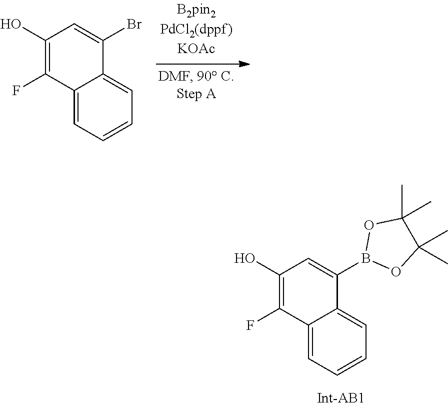 [SMILES: Oc1cc(Br)c2ccccc2c1F ; CC1(C)OB(c2cc(O)c(F)c3ccccc23)OC1(C)C]

4-bromo-1-fluoronaphthalen-2-ol (250 mg, 1.04 mmol), bis(pinacolato)diboron (342 mg, 1.35 mmol), potassium acetate (254 mg. 2.59 mmol) and PdCl₂(dppf)-CH₂Cl₂ adduct (85 mg, 0.10 mmol) in N,N-dimethylformamide (4.0 mL) were sub-surface sparged with nitrogen for 2 minutes and then sealed. The reaction mixture was heated at 90° C. for 4 h. The reaction was then cooled to rt and diluted with ethyl acetate (30 mL). The organic layer was washed with water and 1% aqueous LiCl. The resulting organic layer was filtered through a 1:1 v/v mixture of FLORISIL and anhydrous sodium sulfate and concentrated. The crude mixture was purified by column chromatography on silica gel (0 to 100% EtOAc/hexanes) to yield 1-fluoro-4-(4,4,5,5-tetramethyl-1,3,2-dioxaborolan-2-yl)naphthalen-2-ol (Int-AB1). MS (ESI): m/z (M+H)⁺ 289.

### Intermediate AC2: 1-(tetrahydro-2H-pyran-2-yl)-4-(4,4,5,5-tetramethyl-1,32-dioxaborolan-2-yl)-6-(trifuoromethy)-1H-indazole P 1-A426

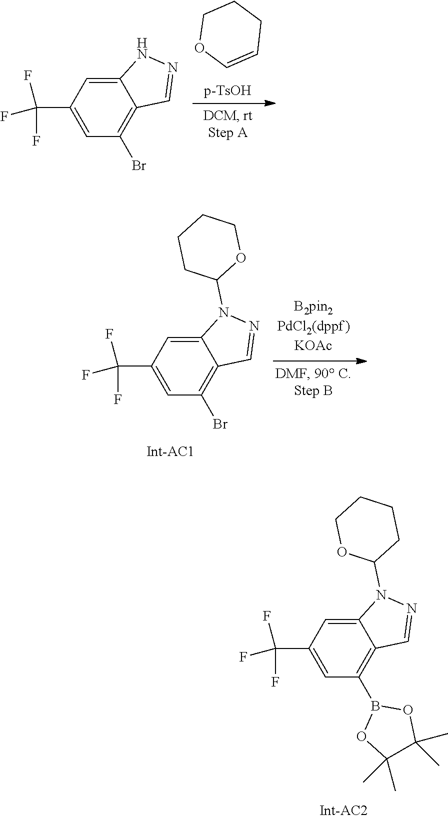

### Step, A: 4-bromo-1-(tetrahydro-2H-pyran-2-yl)-6-(trifluoromethyl)-1H-indazole (Int-AC1)

4-Bromo-6-(trifluoromethyl)-1H-indazole (1.50 g, 5.66 mmol) was dissolved in DCM (10 mL) and treated at 24° C. with 3.4-dihydro-2H-pyran (0.960 mL, 1.1.3 mmol) and p-TsOH (0.108 g, 0.566 mmol). The reaction mixture was stirred at 24° C. for 16 h and then directly loaded onto a silica gel column. The reaction mixture was then purified by column chromatography on silica gel (0 to 50% EtOAc/hexanes) to yield 4-bromo-1-(tetrahydro-2H-pyran-2-yl)-6-(trifluoromethyl)-1H-indazole (Int-AC1). MS (ESI): nm/z (M+H)⁺ 397.

### Step B: 1-(tetrahydro-2H-pyran-2-yl)-4-(4,4,5,5-tetramethyl-1,3,2-dioxaborolan-2-yl)-6-(trifluoromethyl)-1H-indazole (Int-AC2)

4-Bromo-1-(tetrahydro-2H-pyran-2-yl)-6-(trifluoromethyl)-1H-indazole (Int-AC1) (1.00 g, 2.86 mmol), bis(pinacolato)diboron (945 mg, 3.72 mmol), potassium acetate (703 mg, 7.16 mmol) and PdCl₂(dppf)-CH₂Cl₂ adduct (234 mg, 0.286 mmol) in N,N-dimethylformamide (7.0 mL) were sub-surface sparged with nitrogen for 2 minutes and then sealed. The reaction mixture was heated at 90° C. for 4 h. The reaction mixture was then cooled to rt and diluted with ethyl acetate (30 mL). The reaction mixture was then washed with water and 1% aq LiCl. The resulting organic layer was filtered through a 1:1 v/v mixture of FLORISIL and anhydrous sodium sulfate and the filtrate was concentrated. The crude residue was purified by column chromatography on silica gel (0 to 100% EtOAc/hexanes) to afford 1-(tetrahydro-2H-pyran-2-yl)-4-(4,4,5,5-tetramethyl-1,3,2-dioxaborolan-2-yl)-6-(trifluoromethyl)-1H-indazole (Int-AC2). MS (ESI): m % z (M+H)⁺ 397.

### Intermediate AD2: 5-chloro-6-fluoro-1-(tetrahydro-2H-pyran-2-yl)-4-(4,4,5,5-tetramethyl-1,3,2-dioxaborolan-2-yl)-1H-indazole (Int-AD2)

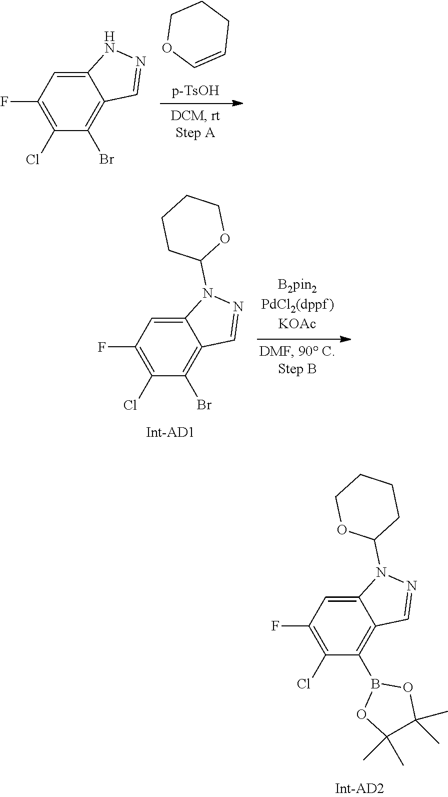

### Step A: 4-bromo-5-chloro-6-fluoro-1-(tetrahydro-2H-pyran-2-yl)-1H-indazole (Int-AD)

4-Bromo-5-chloro-6-fluoro-1H-indazole (1.00 g, 4.01 mmol) in DCM (5.0 mL) was treated at 24° C. with 3.4-dihydro-2H-pyran (0.680 mL, 8.02 mmol) and p-TsOH (76 mg, 0.40 mmol). The reaction mixture was stirred at room temperature for 20 h. The mixture was purified by column chromatography on silica gel (0 to 100% EtOAc/hexanes) to afford 4-bromo-5-chloro-6-fluoro-1-(tetrahydro-2H-pyran-2-yl)-1H-indazole (Int-AD1). 1H NMR (500 MHz, acetone-d₆) δ 8.06 (s, 1H), 7.85-7.66 (m, 1H), 5.86 (dd, J=9.2, 2.5 Hz, 1H), 3.94 (dt, J=11.5, 3.2 Hz, 1H), 3.88-3.72 (m, 1H), 2.57-2.36 (m, 1H), 2.20-2.02 (m, 5H), 1.92-1.75 (m, 2H), 1.69 (dtt, J=22.7, 9.2, 4.7 Hz, 2H), 1.61-1.41 (m, 1H).

### Step B: 5-chloro-6-fluoro-1-(tetrahydro-2H-pyran-2-yl)-4-(4,4,5,5-tetramethyl-1,3,2-dioxaborolan-2-yl)-1H-indazole (Int-AD2)

4-bromo-5-chloro-6-fluoro-1-(tetrahydro-2H-pyran-2-yl)-1H-indazole (Int-AD1) (1.29 g, 3.87 mmol), bis(pinacolato)diboron (1.28 g, 5.03 mmol), potassium acetate (949 mg, 9.67 mmol) and PdCl₂(dppf)-CH₂Cl₂ adduct (316 mg, 0.387 mmol) in N,N-dimethylformanide (7.0 mL) were subsurface sparged with nitrogen for 2 minutes and then sealed. The reaction mixture was heated at 90° C. for 4 h. Then, the reaction mixture was cooled to rt, diluted with ethyl acetate (30 mL), and washed with water and 1% aq LiCl. The layers were separated and the resulting organic phase was filtered through a 1:1 v/v mixture of FLORISIL and anhydrous sodium sulfate and concentrated. The crude residue was purified by column chromatography on silica gel (0 to 100% EtOAc/hexanes) to afford 5-chloro-6-fluoro-1-(tetrahydro-2H-pyran-2-yl)-4-(4,4,5,5-tetramethyl-1,3,2-dioxaborolan-2-yl)-1H-indazole (Int-AD2). MS (ESI): m/z (M+H)⁺ 381.

### Intermediate ADD2: 1-(tetrahydro-2H-pyran-2-yl)-4-(4,4,5,5-tetramethyl-1,3,2-dioxaborolan-2-yl)-5-(trifluoromethyl)- 1H-indazole

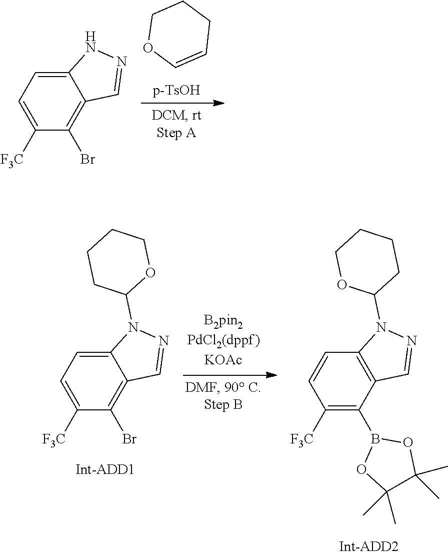

Int-ADD2 was prepared via the same reaction sequence as Int-AC2 and Int-AD2. MS (ESI): m/z (M+H)⁺ 397.

### Intermediate AE1: 6-chloro-5-methyl-1-(tetrahydro-2H-pyran-2-yl)-4-(4,4,5,5-tetramethyl-1,3,2-dioxaborolan-2-yl)-1H-indazole

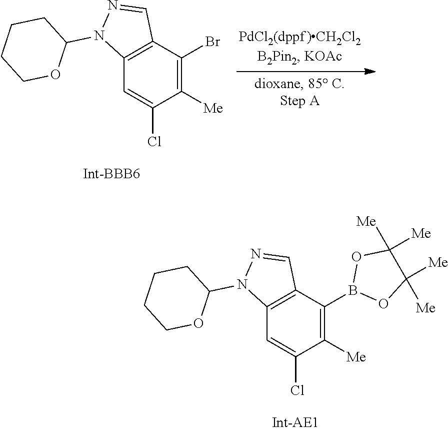 [SMILES: Cc1c(Cl)cc2c(cnn2C2CCCCO2)c1Br ; Cc1c(Cl)cc2c(cnn2C2CCCCO2)c1B1OC(C)(C)C(C)(C)O1]

4-bromo-6-chloro-5-methyl-1-(tetrahydro-2H-pyran-2-yl)-1H-indazole (Int-BBB6) (150 mg, 0.460 mmol), B₂Pin₂ (135 mg, 0.532 mmol), KOAc (142 mg, 1.45 mmol), and PdCl₂(dppf)·CH₂Cl₂ (19 mg, 0.023 mmol) were combined. The reaction vessel was sealed, flushed with nitrogen for 5 min, evacuated in vacuo for 1 min, and backfilled with nitrogen for 1 min. Dioxane (2.30 mL) was added, and the reaction was sparged with nitrogen for 15 min, then backfilled with nitrogen for 1 min. The reaction was heated to 85° C. for 16 h in a microwave reactor. The reaction was cooled to room temperature, diluted with water (10 mL) and extracted with EtOAc (3×10 mL). The combined organic layers were dried over anhydrous MgSO₄, filtered, and the filtrate was concentrated under reduced pressure. The crude residue was purified by silica gel chromatography (gradient elution: 0-75% EtOAc/Hexanes) to provide 6-chloro-5-methyl-1-(tetrahydro-2H-pyran-2-yl)-444,4,5,5-tetramethyl-1,3,2-dioxaborolan-2-yl)-1H-indazole (Int-AE1). MS (ESI): m/z (M+H)⁺ 377.

Int-AF1 in the table below was made via the same procedure as Int-AE1 above, using Int-AAA5 as starting material.

| | | | [M + H]⁺ |
|---|---|---|---|
| Int | Structure | Compound Name | Found |
| Int-AF1 | 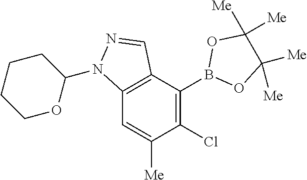 [SMILES: Cc1cc2c(cnn2C2CCCCO2)c(B2OC(C)(C)C(C)(C)O2)c1Cl] | 5-chloro-6-methyl-1- (tetrahydro-2H-pyran-2-yl)- 4-(4,4,5,5-tetramethyl-1,3,2- dioxaborolan-2-yl)-1H- indazole | 377 |

### Intermediate AG1: 5.6-dimethyl-1-(tetrahydro-2H-pyran-2-yl)-4-(trifluoro- ?-boranyl)-1H-indazole, potassium salt (Int-AG)

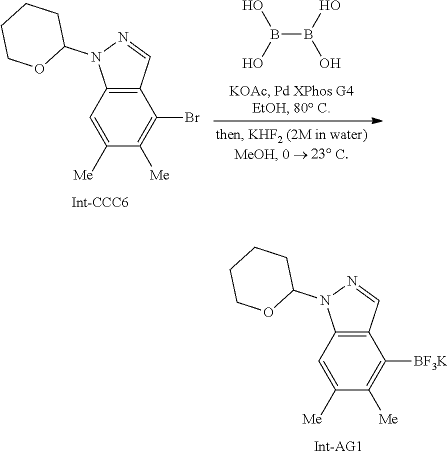

2-Bromo-5,6-dimethyl-1-(tetrahydro-2H-pyran-2-yl)-1H-indazole (Int-CCC6) (1.00 g, 3.23 mmol), tetrahydroxydiboron (435 mg, 4.85 mmol), potassium acetate (952 mg, 9.70 mmol), and XPhos Palladium G4 (278 mg, 0.323 mmol) were combined in a reaction vessel. The reaction vessel was flushed with nitrogen for 5 min, evacuated for 1 min, and backfilled with nitrogen for 1 min. Degassed EtOH (10.8 mL) was added, and the reaction vessel was fitted with a reflux condenser and heated to 80° C. for 2 h. The reaction was cooled to room temperature, concentrated under reduced pressure and the residue was re-suspended in 10 mL EtOAc and filtered through CELITE. The filter pad was flushed with EtOAc (5×5 mL), and the combined filtrate was concentrated under reduced pressure. The crude residue was dissolved in MeOH (˜15 mL) and cooled to 0° C. Potassium hydrogen difluoride (7.0 mL, 21.0 mmol) was added dropwise. The reaction was stirred vigorously in a melting ice bath overnight. The reaction was concentrated under reduced pressure, diluted with −5 mL of MeCN and lyophilized. The lyophilized mixture was dissolved in acetone and filtered through CELITE. The reaction was concentrated under reduced pressure and triturated with ether to provide 5,6-dimethyl-1-(tetrahydro-2H-pyran-2-yl)-4-(trifluoro-λ⁴-boranyl)-1H-indazole, potassium salt (Int-AG1). ¹H NMR (600 MHz, DMSO-d6) δ 8.04 (s, 1H), 7.13 (s, 1H), 5.62 (dd, J=9.7, 2.2 Hz, 1H), 3.87 (d, J=11.2 Hz, 1H), 3.73-3.64 (m, 1H), 3.44-3.36 (m, 1H), 2.46-2.35 (m, 1H), 2.29 (d, J=5.2 Hz, 6H), 2.03 (d, J=13.3 Hz, 1H), 1.91-1.84 (m, 1H), 1.73 (dd, J=14.1, 6.1 Hz. 1H), 1.60-1.52 (m, 1H).

### Intermediate AH7: (2-(2.4-dimethoxybenayl)amino)-5-fluoro-7-methylquinolin-8-yl)boronic acid (Int-AH7)

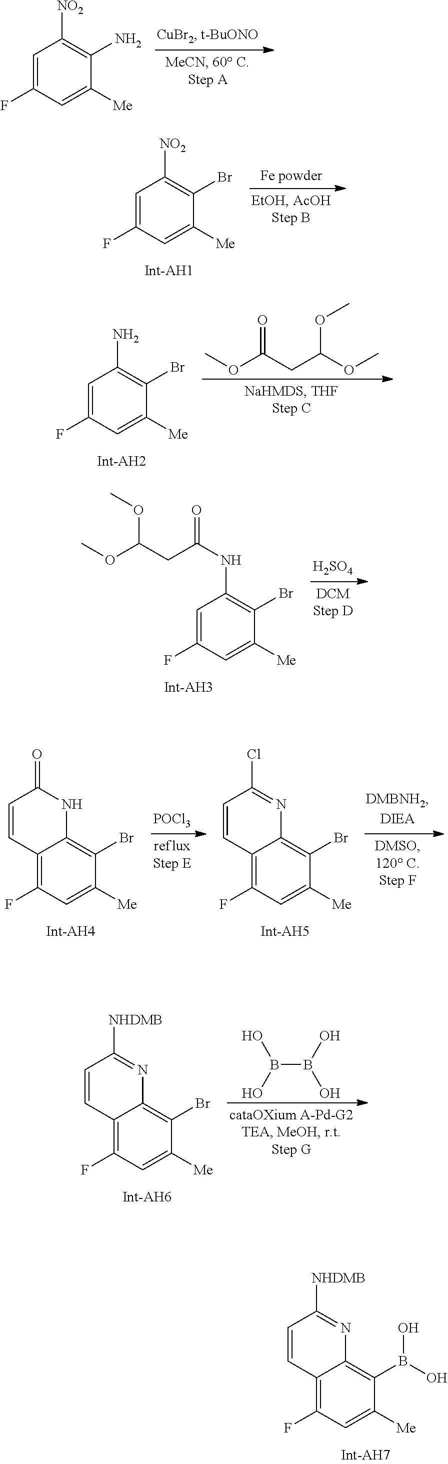

### Step A: 2-bromo-5-fluoro-1-methyl-3-nitrobenzene (Int-A A)

To a mixture of copper (II) bromide (31.5 g, 141 mmol) and tert-butyl nitrite (21.1 mL, 176 mmol) in acetonitrile (15.0 mL) was added 4-fluoro-2-methyl-6-nitroaniline (20.0 g, 118 mmol) dissolved in acetonitrile (80 mL) at 0° C. After stirring at 0° C. for 0.5 h, the reaction mixture was heated to 60° C. for 16 h. After cooling to room temperature, the reaction mixture was extracted with ethyl acetate (100 mL×3) and washed with saturated brine. The combined organic fractions were dried over anhydrous Na₂SO₄, filtered, and concentrated under vacuum. The residue was purified by flash silica gel chromatography (Pet, ether) to give 2-bromo-5-fluoro-1-methyl-3-nitrobenzene (Int-A1H₁). ¹H NMR (400 MHz, CDCl₃) δ 7.31 (dd, J=2.7, 7.3 Hz, 1H), 7.22 (dd, J=2.8, 8.4 Hz, 1H), 2.53 (s, 3H).

### Step B: 2-bromo-5-fluoro-3-methylaniline (Int-AH2)

To a solution of 2-bromo-5-fluoro-1-methyl-3-nitrobenzene (Int-AH1) (25.0 g, 107 mmol) in ethanol (250 mL) and acetic acid (125 mL) was added iron powder (29.8 g, 534 mmol). The reaction mixture was stirred at 60° C. for 6 h. The reaction mixture was filtered and the filtrate was concentrated under reduced pressure. The residue was purified by flash silica gel chromatography (Eluent of 0-3% EtOAc/Pet, ether) to give 2-bromo-5-fluoro-3-methylaniline (Int-AH2). MS (ESI) [M+H]⁺ m/z 203, 205.

### Step C: N-(2-bromo-5-fluoro-3-methylphenyl)-3,3-dimethoxypropanamide (Int-AH3)

To a solution of 2-bromo-5-fluoro-3-methylaniline (Int-AH2) (4.90 g, 24.0 mmol) and methyl 3.3-dimethoxypropanoate (4.27 g, 28.8 mmol) in THF (30 mL) was added NaHMDS (36.0 mL, 36.0 mmol) (1 M in THF) at 0° C. under N₂ protection dropwise over 10 min. The reaction mixture was stirred at 0° C. for 30 min. Then the reaction mixture was warmed to 20° C. and stirred at this temperature for 12 h. The reaction mixture was quenched with water (30 mL) and the resulting mixture was extracted with EtOAc (70 mL×3). The combined organic phase was dried over anhydrous Na₂SO₄, filtered and concentrated in vacuo. The residue was purified by flash silica gel chromatography (Pet, ether/EtOAc=10/1) to give N-(2-bromo-5-fluoro-3-methylphenyl)-3,3-dimethoxypropanamide (Int-AH3). ¹H NMR (400 MHz, CDCl₃) δ 9.05-8.91 (m, 1H), 8.21-8.04 (m, 1H), 6.74 (dd, J=2.8, 8.4 Hz, 1H), 4.76 (m, 1H), 3.47 (s, 6H), 2.79 (d, J=5, 1 Hz, 2H), 2.41 (s, 3H).

### Step D: 8-bromo-5-fluoro-7-methylquinolin-2(1H)-one (Int-AH4)

To solution of N-(2-bromo-5-fluoro-3-methylphenyl)-3,3-dimethoxypropanamide (Int-AH3) (5.00 g, 15.6 mmol) in DCM (50 mL) was added concentrated H₂SO₄ (12.5 mL, 234 mmol) at 0° C. The reaction mixture was warmed to 20° C. and stirred at 20° C. for 3 h. The reaction mixture was concentrated under reduced pressure and the residue was added to ice water and extracted with DCM (80 mL×3). The combined organic layers were washed with brine (30 mL), dried over anhydrous Na₂SO₄ and filtered. The filtrate was concentrated under reduced pressure to give 8-bromo-5-fluoro-7-methylquinolin-2(1H)-one (Int-AH4). MS (ESI) [M+H]⁺ m/z: 257.

### Step E: 8-bromo-2-chloro-5-fluoro-7-methylquinoline (Int-AH5)

A solution of 8-bromo-5-fluoro-7-methylquinolin-2(H)-one (Int-AH4) (4.80 g, 18.74 mmol) in POCl₃ (50.0 mL, 536 mmol) was stirred at 110° C. for 4 h. The reaction mixture was concentrated under reduced pressure to give a residue, which was purified by flash silica gel chromatography (Eluent of 0-36% EtOAc/Pet, ether gradient) to give 8-bromo-2-chloro-5-fluoro-7-methylquinoline (Int-AH5). ¹H NMR (400 MHz, CDCl₃) δ 8.46 (d, J=8.6 Hz, 1H), 7.57 (d, J=8.6 Hz. 1H). 7.38 (d, J=10.2 Hz, 1H), 2.67 (s, 3H).

### Step F: 8-bromo-N42, 4-dimethoxybenzyl)-5-fluoro-7-methylquinolin-2-amine (Int-AH6)

To a solution of 8-bromo-2-chloro-5-fluoro-7-methylquinoline (Int-AH5) (3.50 g, 12.8 mmol) in DMSO (35 mL) were added (2,4-dimethoxyphenyl) methanamine (2.87 mL, 19.1 mmol) and N,N-diisopropylethylamine (6.68 mL, 38.2 mmol). The reaction mixture was stirred at 120° C. for 3 h. The reaction mixture was quenched with water (50 mL) and extracted with EtOAc (90 mL×3). The combined organic layer was dried over anhydrous Na₂SO₄ and filtered. The filtrate was concentrated and purified by flash silica gel chromatography (Eluent of 0-10% EtOAc/Pet, ether gradient) to give 8-bromo-N-(2,4-dimethoxybenzyl)-5-fluoro-7-methylquinolin-2-amine (Int-AH6). MS (ESI) [M+H]⁺ m/z: 405, 407.

### Step G: (24(2, 4-dimethoxybenzyl) amino)-5-fluoro-7-methylquinolin-8-yl) boronic acid (Int-AH7)

To a solution of 8-bromo-N-(2,4-dimethoxybenzyl)-5-fluoro-7-methylquinolin-2-amine (Int-AH6) (500 mg, 1.234 mmol) in MeOH (10 mL) were added hypodiboric acid (442 mg, 4.94 mmol), TEA (0.516 mL, 3.70 mmol) and chloro [(di(1-adamantyl)-n-butylphosphine)-2-(2-aminobiphenyl)] palladium (II) (165 mg. 0.247 mmol) at 25° C. The mixture was sparged with nitrogen for 1 min and stirred at 80° C. for 3 h. The mixture was filtered and the filtrate was concentrated under reduced pressure to give the residue which was purified by Prep-TLC (SiO₂, Pet, ether/EtOAc=2/1, v/v) to give (2-((2, 4-dimethoxybenzyl) amino)-5-fluoro-7-methylquinolin-8-yl) boronic acid (Int-AH7). MS (ESI) [M+H]⁺ m/z: 371.

### Intermediate AI10: 4-(tert-butoxy)-6,8-difluoro-2-(((2R,7aS)-2-fluorotetrahydro-1H-pyrrolizin-7a(5H)-yl))methoxy)quinazoline (Int-AI10)

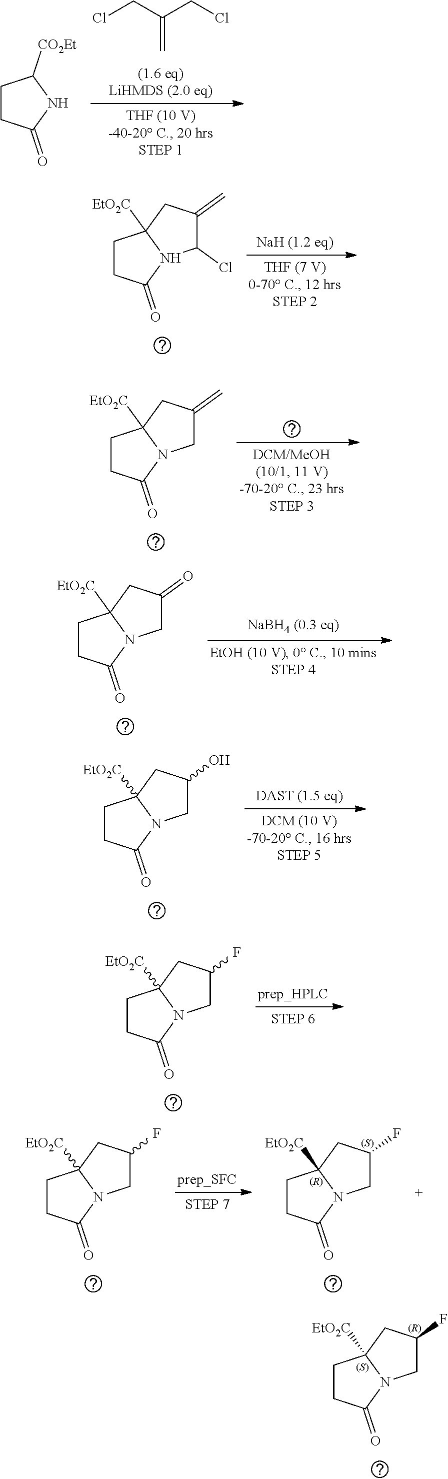

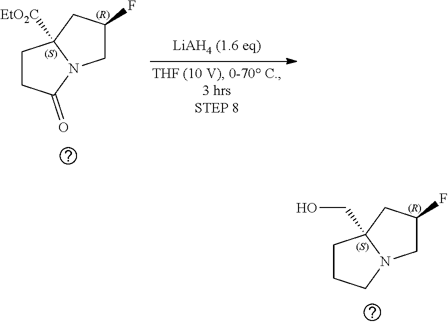

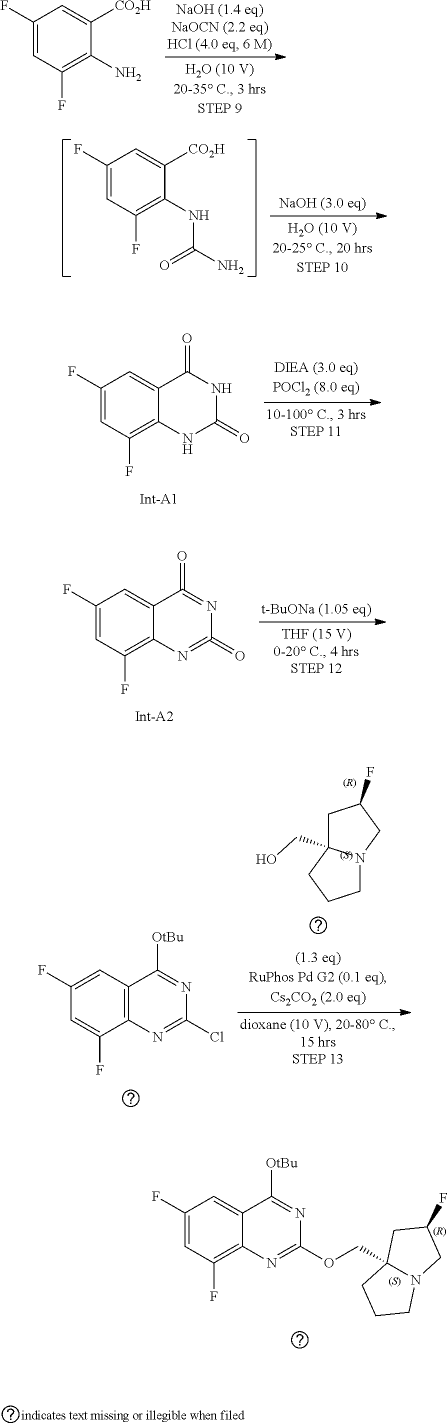

### STEP 1-Ethyl 242-(chloromethyl)allyl)-5-oxopyrrolidine-2-carboxylate (Int-AI1)

To ethyl -5-oxopyrrolidine-2-carboxylate (200) g. 1.27 mol, 1.00 eq) and 3-chloro-2-(chloromethyl)prop-1-ene (254 g, 2.04 mol, 235 mL, 1.60 eq) in THF (2.00 L) at −40° C. under N2, LiHMDS (1.00 M, 2.55 L, 2.00 eq) was added drop-wise and the reaction stirred at 20° C. for 20 hrs. The reaction mixture was poured into sat. NH₄Cl solution (1.00 L), pH was adjusted between 6-7 with 1 N HCl. The reaction mixture was extracted with EtOAc (500 mL×3). The combined organic layers were washed with brine (600 mL) and the residue was concentrated under reduced pressure. This was purified on a silica gel column using Petroleum ether/Ethyl acetate=50/1 to 1/1) to give ethyl 2-(2-(chloromethyl)allyl)-5-oxopyrrolidine-2-carboxylate.

### STEP 2: Ethyl-2-methylene-5-oxotetrahydro-1H-pyrrolizine-7a(5H)-carboxylate (Int-A12)

To a solution of ethyl 2-(2-(chloromethyl)allyl)-5-oxopyrrolidine-2-carboxylate (Int-AI-1) (250 g, 1.02 mol, 1.00 eq) in THF (250 mL), NaH (58.6 g, 1.22 mol, 50% purity, 1.20 eq) in THF (1.50 L) was added drop-wise at 0° C. under N₂ and the reaction mixture was stirred at 70° C. for 12 hrs under N₂. The reaction mixture was poured into sat. NH₄Cl solution (1.00 L) and stirred at 5° C. for 1 hr. The reaction mixture was extracted with EtOAc (300 mL×3). The combined organic layer was washed with brine (500 mL×2), dried over anhydrous Na₂SO₄, filtered, and the resulting mixture was concentrated under reduced pressure. The residue obtained was purified on a silica gel column using Petroleum ether/Ethyl acetate=50/1 to 1/1 to give ethyl-2-methylene-5-oxotetrahydro-1H-pyrrolizine-7a(5H)-carboxylate.

### STEP 3: Ethyl 2.5-dioxotetrahydro-1H-pyrrolizine-7a(5H)-carboxylate (Int-A13)

O₃ (0.5-1 m³/h) was bubbled into a solution of ethyl-2-methylene-5-oxotetrahydro-1H-pyrrolizine-7a(5H)-carboxylate(Int-A12) (155 g, 0.740 mol, 1.00 eq) in DCM (1.55 L) and MeOH (155 mL) at −70° C. for 9 hrs. The resulting solution was pale blue. N2 was bubbled to purge excess 03, and then Me₂S (73.6 g, 1.19 mol, 87.0 mL, 1.60 eq) was added to the reaction mixture at −70° C. and the reaction mixture was stirred at 20° C. for 14 hrs. The reaction mixture was concentrated under reduced pressure and the residue obtained was purified on a silica gel column using Petroleum ether/Ethyl acetate=10/1 to 1/1 to give ethyl 2,5-dioxotetrahydro-1H-pyrrolizine-7a(5H)-carboxylate.

### STEP 4: Ethyl 2-hydroxy-5-oxotetrahydro-1H-pyrrolizine-7a(5H)-carboxylate (Int-A4)

NaBH₄ (5.64 g, 149 mmol, 0.30 eq) was added to a solution of ethyl 2,5-dioxotetrahydro-1H-pyrrolizine-7a(5H)-carboxylate (Int-A13) (105 g, 497 mmol, 1.00 eq) in EtOH (1.05 L) portion-wise at 0° C. under N₂ and the reaction mixture was stirred at 0° C. for 10 mins. The reaction mixture was quenched with HCl (1.00 mol/L, 5.5 mL) at 5° C. and stirred at 5° C. for 0.5 hr. The reaction mixture was concentrated under reduced pressure and the residue obtained was purified on a silica gel column using Petroleum ether/Ethyl acetate=50/1 to 1/1) to give ethyl 2-hydroxy-5-oxotetrahydro-1H-pyrrolizine-7a(5H)-carboxylate.

### STEP 5 and STEP 6: Ethyl-2-fluoro-5-oxotetrahydro-1H-pyrrolizine-7a(5H)-carboxylate (Int-A15 and Int-A16)

To a solution of ethyl 2-hydroxy-5-oxotetrahydro-1H-pyrrolizine-7a(5H)-carboxylate (Int-A14) 100 g, 468 mmol, 1.00 eq) in DCM (1000 mL) at −70° C. under N₂. DAST (113 g, 703 mmol, 92.9 mL, 1.50 eq) was added drop-wise. The reaction mixture was warmed to 20° C. and stirred at 20° C. for 16 hrs. The reaction was quenched with EtOH (50.0 mL) at 10° C. The reaction mixture was diluted with water (900 mL) and then extracted with DCM (900 mL×2). The combined organic layers were washed with brine (900 mL), dried over anhydrous Na₂SO₄, filtered, and the resulting mixture was concentrated under reduced pressure. The residue obtained was purified on a silica gel column (Petroleum ether/Ethyl acetate=20/1 to 1/1) to give an oil (320 g). This oil was further purified by prep HPLC (column: Welch Xtimate C18 250×70 mm; mobile phase: [water (0.05% NH₃H₂O) -ACN]; B %: 5%-30%, 20 mins) to give ethyl-2-fluoro-5-oxotetrahydro-1H-pyrrolizine-7a(5H)-carboxylate.

### STEP 7: Separation of ethyl (2S,7aR)-2-fluoro-5-oxotetrahydro-1H-pyrrolizine-7a(5H)-carboxylate and ethyl (2R,7aS)-2-fluoro-5-oxotetrahydro-1H-pyrrolizine-7a(5H)-carboxylate (Int-AI7a and Int-A17b)

The residue obtained was resolved on SFC (column: DAICEL CHIRALPAK AS (250mmx50 mm, 10 um); mobile phase: [0.1% NH₃H₂O IPA]: B %: 20%-20%,5 mins) to give compound Int-A17a (peak 1, ethyl (2S,7aR)-2-fluoro-5-oxotetrahydro-1H-pyrrolizine-7a(5H)-carboxylate) and compound Int-AI7b (peak 2, ethyl (2R,7aS)-2-fluoro-5-oxotetrahydro-1H-pyrrolizine-7a(5H)-carboxylate).

### STEP 8: ((2R7aS)-2-fluorotetrahydro-1H-pyrrolizin-7a(5H)-yl)methanol (Int-AIR)

A solution of ethyl (2R,7aS)-2-fluoro-5-oxotetrahydro-1H-pyrrolizine-7a(5H)-carboxylate (Int-A17b) (84.5 g, 392 mmol, 1.00 eq) in THF (255 mL) was added dropwise to a mixture of LAH (22.3 g, 588 mmol, 1.50 eq) in THF (595 mL) at 0° C. under N₂. The reaction mixture was warmed to 70° C. and stirred for 3 hrs. The reaction mixture was cooled to 0° C. and quenched with Na₂SO₄.10 H₂O at 0° C. under N₂. The reaction mixture was stirred for 30 mins at 20° C. and then filtered. The filter cake was washed with EtOAc (600 mL×5), dried over anhydrous Mg₂SO₄, and filtered. The filtrate was concentrated under reduced pressure to give ((2R,7aS)-2-fluorotetrahydro-1H-pyrrolizin-7a(5H)-yl)methanol. The crude product was directly used in the next step without further purification.

For the purpose of this disclosure, every description of ((2R,7aS)-2-fluorotetrahydro-1H-pyrrolizin-7a(5H)-yl)methanol (Int-A18) compound used had the depicted stereochemistry, confirmed by X-ray and may not be explicitly written on every structure.

### STEP 9: 6,8-difluoroquinazoline-2,4(1H,3H)-dione (Int-A1) via 3.5-difluoro-2-ureidobenzoic acid

A solution of NaOH (32.3 g, 808 mmol, 1.40 eq) in H₂O (1.00 L) was added into a 3.00 L three-necked round bottom flask at 20° C. 2-amino-3,5-difluorobenzoic acid (100 g, 577 mmol, 1.00 eq) was added into the mixture at 20° C. The reaction mixture was warmed to 35° C. for 1 hr. To this mixture sodium cyanate (82.6 g, 1.27 mol, 2.20 eq) was added and the reaction mixture was stirred at 35° C. until the solids dissolved. Then, slowly dropwise, HCl/H₂O (6.00 M. 385 mL, 4.00 eq) was added to the reaction mixture for over an hr and the pH was maintained between 6-7.

### Step 10: 6,8-difluoroquinazoline-2,4(1H,3H)-dione (Int-A1) via 3.5-difluoro-2-ureidobenzoic acid (continued)

NaOH (69.3 g, 1.73 mol, 3.00 eq) was added into the reaction mixture at 20° C. and the reaction mixture was stirred at 25° C. for 12 hrs. The pH of the reaction mixture was adjusted between 2-3 by addition of HCl (12.0 mol/L) at 20° C. To this mixture, acetone (500 mL) was added and the mixture was stirred at 25° C. for 1 hr and then filtered. The filter cake was washed with acetone (150 mL×2), dried in vacuo to give 6.8-difluoroquinazoline-2,4(1H,3H)-dione.

### STEP 11: 2,4-dichloro-6,8-difluoroquinazoline (Int-A2)

6,8-difluoroquinazoline-2,4(1H,3H)-dione (Int-A1) (83 g, 418 mmol, 1.00 eq) and POCl₃ (513 g, 3.35 mol, 311 mL, 8.00 eq) were added into a 1.00 L three-necked round bottom flask at 20° C. DIEA (162 g, 1.26 mol, 218 mL, 3.00 eq) was added to the above mixture at 10° C. The reaction mixture was then warmed to 100° C. for 3 hrs. The residue was dissolved in EtOAc (700 mL) and washed with aq. sat. NaHCO₃(350 mL). The organic layer was separated and washed with brine (300 mL) and dried over anhydrous Na₂SO₄. Excess solvent was removed under reduced pressure. The crude product was triturated with MTBE (200 mL) at 25° C. for 30 mins then filtered. The filter cake dried under vacuum to give 2,4-dichloro-6,8-difluoroquinazoline.

### STEP 12: 4-(tert-butoxy)-2-chloro-6,8-difluoroquinazoline (Int-A19)

A solution of t-BuONa (33.9 g, 352 mmol, 1.05 eq) in THF (400 mL) was added to the mixture of 2,4-dichloro-6,8-difluoroquinazoline (Int-A2) (79.0 g, 336 mmol, 1.00 eq) in THF (800 mL) at 0° C. drop-wise. The reaction mixture was stirred for 4 hrs at 20° C. The reaction mixture was diluted with EtOAc (1.00 L) and washed with water (500 mL) and brine (500 mL). The organic layer was separated and concentrated under reduced pressure and the residue obtained was purified on a silica gel column using Petroleum ether/Ethyl acetate=100/1 to 10/1 to give 4-(tert-butoxy)-2-chloro-6,8-difluoroquinazoline.

### STEP 13: 4-(tert-butoxy)-6,8-difluoro-2-(((2R,7aS)-2-fluorotetrahydro-1H-pyrrolizin-7a(5H)-yl)methoxy)quinazoline (Int-AI10)

To the solution of 4-(tert-butoxy)-2-chloro-6,8-difluoroquinazoline(Int-A19) in dioxane (50.0 mL), ((2R,7aS)-2-fluorotetrahydro-1H-pyrrolizin-7a(5H)-yl)methanol (Int-AI8) (3.79 g, 23.8 mmol, 1.30 eq) and Cs₂CO₃ (11.9 g, 36.6 mmol, 2.00 eq) and RuPhos Pd G2 (1.42 g, 1.83 mmol, 0.10 eq) were added to the reaction mixture at 20° C. under N₂. The reaction mixture was warmed to 80° C. for 15 hrs under N₂. The reaction mixture was then cooled to room temperature and filtered. The filter cake was washed with EtOAc (15.0 mL×3). The filtrate was concentrated under reduced pressure and the residue obtained was purified on a silica gel column using Petroleum ether/Ethyl acetate=100/1 to 5/1 to give 4-(tert-butoxy)-6,8-difluoro-2-(((2R,7aS)-2-fluorotetrahydro-1H-pyrrolizin-7a(5H)-yl)methoxy)quinazoline. ¹H NMR. (400 MHz, METHANOL-d₄) δ 7.43-7.52 (m, 2H), 5.21-5.44 (m, 1H), 4.20-4.37 (m, 2H), 3.21-3.30 (m, 2H), 3.19 (s, 1H), 2.97-3.09 (m, 1H), 2.20-2.42 (m, 2H), 2.10-2.20 (m, 1H), 1.85-2.07 (m, 3H), 1.78 (s, 9H). MS (ESI): m/z (M+H)⁺ 396.1.

### Intermediate AJ: Tert-butyl (1S,5S)-3-benzyl-1-fluoro-5-methyl-3,8-diazabicyclo[3.2.1]octane-8-carboxylate (Int-AJ=1)

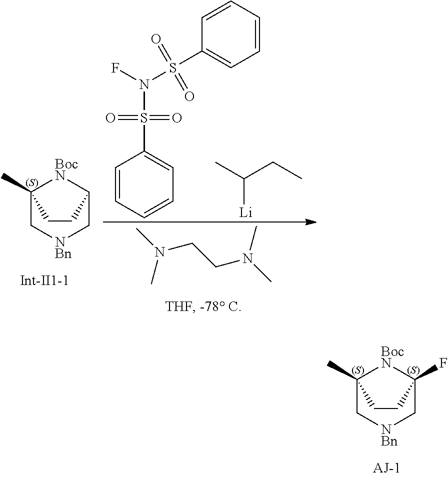

To a solution of tert-butyl (1R,5S)-3-benzyl-1-methyl-3,8-diazabicyclo[3.2.1]octane-8-carboxylate (Int-II1-1) (200 mg, 0.632 mmol) and TMEDA (0.214 mL, 1.416 mmol) in THF (5 mL) was added sec-butylthium (1.215 mL, 1.580 mmol) (1,3 M in Hexane) dropwise over 5 min at −78° C. under N2 protection. The reaction mixture was stirred at −78° C. for 1 h. Then N-fluorobenzenesulfonimide (399 mg, 1.264 mmol) in 5 mL THF was added dropwise. The reaction mixture was stirred at −78° C. for another 5 min. The reaction was quenched with the solution of NH₄Cl. The residue was purified by Prep-HPLC (Column:Boston Prime C18 150*30 mm*5 um; Condition:water(0.05% NH₃H₂O+10 mM NH₄HCO3)-ACN to give tert-butyl (1S,5S)-3-benzyl-1-fluoro-5-methyl-3,8-diazabicyclo[3.2.1]octane-8-carboxylate. MS (ESI): m/z (M+H)⁺ 335.

### Intermediate ALS: ((7aR)-2-(difluoromethyl)tetrahydro-1H-pyrrolizin-7a(5H)-yl)methanol (Int-ALS)

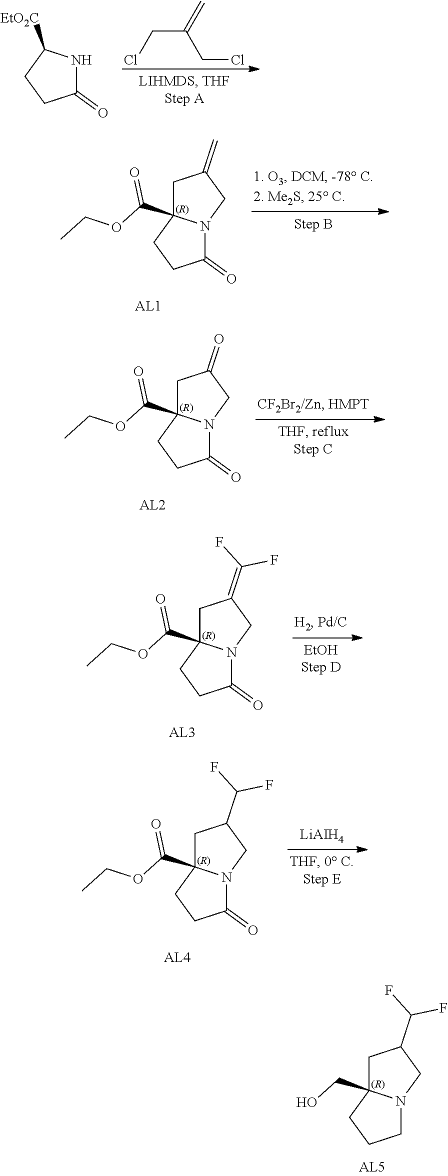

### Step A: Ethyl (R)-2-methylene-5-oxotetrahydro-1H-pyrrolizine-7a(5H)-carboxylate (Int-AL1)

To a mixture of D-pyroglutamic acid ethyl ester (10 g, 63.6 mmol) and 3-chloro-2-chloromethyl-1-propene (31.8 g, 255 mmol) in THF (150 mL) was added LiHMDS (127 mL, 127 mmol) (1 M in THF) at −40° C. under N₂. The reaction mixture was stirred at −40° C. for 2 h. The reaction mixture was quenched with sat. NH₄Cl, and extracted with EtOAc (100 mL×3). The combined organic layers were washed with brine (80 mL), dried over Na₂SO₄, filtered, and concentrated under reduced pressure to give the crude product, which was purified by flash silica gel chromatography 120 g column, Eluent of 0-30% ethyl acetate in petroleum ether gradient to give ethyl (R)-2-methylene-5-oxotetrahydro-1H-pyrrolizine-7a(5H)-carboxylate. ¹H NMR (500 MHz, CDCl₃) δ 5.08-4.99 (m, 2H), 4.26 (br d, J=15.6 Hz, 1H), 4.17 (q, J=7.1 Hz, 2H), 3.70 (br d, 1=15.6 Hz, 1H), 3.03 (br d, J=15.7 Hz, 1H), 2.75 (td, J=10.0, 16.7 Hz, 1H), 2.58 (ddd, J=1.7, 9.2, 13.1 Hz, 1H), 2.50-2.38 (m, 2H), 2.17-2.06 (m, 1H), 1.24 (t, J=7.1 Hz. 3H).

### Step B: Ethyl (R-2,5-dioxotetrahydro-1H-pyrrolizine-7a(5H)-carboxylate (Int-AL2)

A solution of ethyl (R)-2-methylene-5-oxotetrahydro-1H-pyrrolizine-7a(5H)-carboxylate (Int-AL) (3 g, 14.34 mmol) in DCM (30 mL) was bubbled with O₃ at −78° C. until the mixture was turned to blue. After the reaction completion, dimethylsulfane (1.782 g, 28.7 mmol) was added to the reaction mixture and the mixture was stirred at 25° C. for 16 h. Reaction was monitored using LC. The reaction mixture was diluted with water (20 mL) and extracted with EtOAc (40 mL×3). The combined organic layers were washed with brine (30 mL), dried over Na₂SO₄, filtered, and concentrated under reduced pressure to give the crude product, which was purified by flash silica gel chromatography 40 g column, Eluent of 0-50% ethyl acetate in petroleum ether gradient to give ethyl (R)-2,5-dioxotetrahydro-1H-pyrrolizine-7a(5H)-carboxylate. MS (ESI): m/z (M+H)⁺ 212.

### Step C: Ethyl (R)-2-(difluoromethylene)-5-oxotetrahydro-1H-pyrrolizine-7a(5H)-carboxylate (Int-AL3)

Dibromodifluoromethane (1.946 mL, 21.31 mmol) was added to a solution of ethyl (R)-2,5-dioxotetrahydro-1H-pyrrolizine-7a(5H)-carboxylate (Int-AL2) (1.5 g, 7.10 mmol) and HMPT (3.91 mL, 21.31 mmol) in THF (20 mL) at 0° C. The reaction mixture was warmed to room temperature and zinc powder (1,393 g, 21.31 mmol) was added. The reaction mixture was stirred at 80° C. for 3.5 h. The reaction was monitored using LCMS, The mixture was diluted with water (20 mL) and extracted with EtOAc (40 mL×3). The combined organic layers were washed with brine (30 mL), dried over Na₂SO₄, filtered, and concentrated under reduced pressure to give the crude product, which was purified by flash silica gel chromatography 40 g column, Eluent of 0-50% ethyl acetate in petroleum ether gradient to give ethyl (R)-2-(difluoromethylene)-5-oxotetrahydro-1H-pyrrolizine-7a(5H)-carboxylate. MS (ESI) m/z: (M+H)⁺ 246.

¹H NMR (400 MHz, CDCl₃) δ 4.35 (br d, J=14.5 Hz, 1H), 4.23 (q, J=7.0 Hz, 2H), 3.74 (br d, J=14.1 Hz, 1H), 3.13 (br d, J=15.7 Hz, 1H), 2.84-2.75 (m, 1H). 2.62 (ddd, J=2.0, 9.2, 13.1 Hz. 1H), 2.47 (ddd, J=2.0, 9.6, 16.6 Hz, 1H), 2.42-2.34 (m, 1H), 2.14 (td, J=10.0, 13.2 Hz, 1H), 1.29 (t, J=7.0 Hz, 3H).

### Step D: Ethyl (7aR)-2-(difluoromethyl)-5-oxotetrahydro-1H-pyrrolizine-7a(5H)-carboxylate (Int-AL4)

To a solution of ethyl (R)-2-(difluoromethylene)-5-oxotetrahydro-1H-pyrrolizine-7a(5H)-carboxylate (Int-AL3) (412 mg, 1.680 mmol) in EtOH (10 mL) was added dry Pd-C(89 mg, 0.840 mmol). The mixture was stirred at 15° C. for 16 h under H₂ with hydrogen balloon (15 psi). The reaction was monitored using LC. The reaction mixture was filtered, washed with EtOH (15 mL), and the solvent was evaporated under reduced pressure to give the crude product ethyl (7aR)-2-(difluoromethyl)-5-oxotetrahydro-1H-pyrrolizine-7a(5H)-carboxylate, which was used directly in the next step without further purification. MS (ESI), m/z (M+H)⁺ 248. ¹H NMR (400 MHz, CDCl₃) δ 5.98-5.55 (m, 1H), 4.23 (q, J=7.2 Hz, 2H), 4.06 (dd, J=8.8, 12.3 Hz, 1H), 3.06 (dd, J=7.0, 12.5 Hz, 1H), 2.93-2.69 (m, 2H), 2.64-2.52 (m, 2H), 2.42 (br dd, J=8.8, 15.8 Hz, 1H), 2.12-1.92 (m, 2H), 1.29 (t, J=7.2 Hz, 3H).

### Step E: ((7aR)-2-(difluoromethyl)tetrahydro-1H-pyrrolizin-7a(5H)-yl)methanol (Int-ALS)

To a suspension of LiAlH₄ (230 mg, 6.07 mmol) in THF (5 mL) at 0° C. under nitrogen was added dropwise a solution of ethyl (7aR)-2-(difluoromethyl)-5-oxotetrahydro-1H-pyrrolizine-7a(5H)-carboxylate (750 mg, 3.03 mmol) in THF (5 mL). The reaction mixture was heated at 20° C. for 10 min. The mixture was cooled to 0° C. and was slowly diluted with water (0.2 mL), 15% aq NaOH (1 mL) and water (0.3 mL). The reaction mixture was stirred at 0PC for 5 mins and filtered, and the filtrate was concentrated under reduced pressure to give ((7aR)-2-(difluoromethyl)tetrahydro-1H-pyrrolizin-7a(5H)-yl)methanol. The crude product was used in the next step without further purification. ¹H NMR (400 MHz, DMSO-d₆) δ 6.24-5.74 (m, 1H), 4.51 (br t, J=5.7 Hz, 1H), 3.12 (m, 1H),3.07-2.99 (m, 1H), 2.94-2.83 (m, 1H) 2.75-2.67 (m, 2H), 2.66-2.53 (m. 1H), 1.87-1.76 (m, 1H), 1.73-1.62 (m, 3H), 1.60-1.49 (m, 1H), 1.47-1,36 (m, 1H).

### Intermediate AM6: ((3S,8aS)-7.7-difluorooctahydroindolizin-3-yl)methanol (Int-AM6)

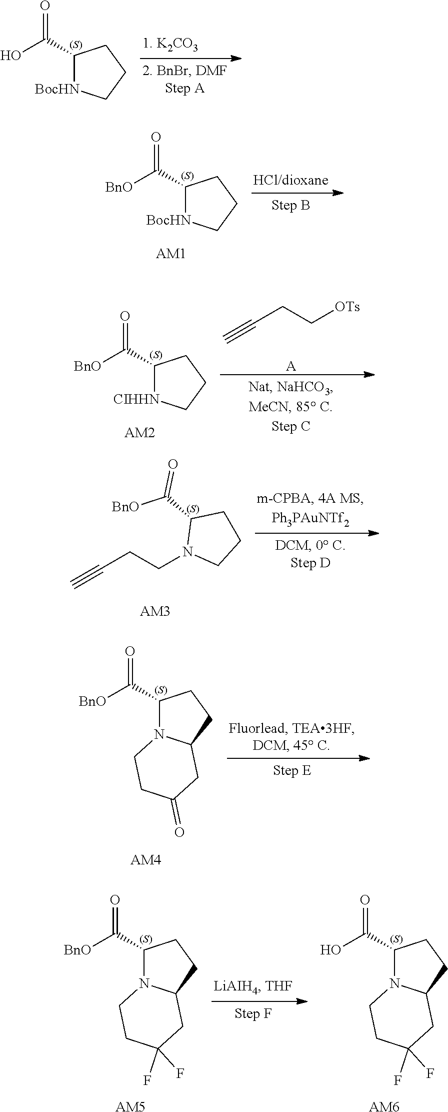

### Step A: 2-benzyl-(tert-butyl) (S)-pyrrolidine-1,2-dicarboxylate (Int-AM1)

To the solution of Boc-L-proline (2 g, 9.29 mmol) in DMF (15 ml) was added K₂CO₃ (3.85 g, 27.9 mmol) and benzyl bromide (1.216 ml, 10.22 mmol), the resulting mixture was stirred at rt overnight. The mixture was partitioned between EtOAc (200 mL) and brine (200 mL), the organic phase was further washed with brine (2×200 mL), dried over Na₂SO₄, concentrated and the residue was purified on silica gel column (120g) using 0-40% EtOAc/hexane as eluting solvents to give 2-benzyl 1-(tert-butyl) (S)-pyrrolidine-1,2-dicarboxylate. MS (ESI): m/z (M+H)⁺ 306.

### Step B: Benzyl L-prolinate (Int-AM2)

2-benzyl 1-(tert-butyl) (S)-pyrrolidine-1,2-dicarboxylate (Int-AM1) (1.5 g, 4.91 mmol) was dissolved in HCl/dioxane (4 M in dioxane) (10 mL), and the reaction mixture was stirred at 20° C. for 1 h. The reaction mixture was concentrated under vacuum to afford benzyl L-prolinate MS (ESI): m/z (M+H)⁺ 206.

### Step C: Benzyl but-3-yn-1-yl-L-prolinate (Int-AM3)

To a solution of benzyl L-prolinate (Int-AM2) (4.77 g, 23.24 mmol), sodium iodide (1.742 g, 11.62 mmol) and sodium bicarbonate (7.81 g, 93 mmol) in MeCN (70 mL) was added 3-butynyl p-toluenesulfonate (5.34 mL, 30.2 mmol). The reaction mixture was stirred at 85° C. for 12 h. The reaction mixture was cooled, diluted with EtOAc (400 mL), washed with H₂O (30 mL), brine (50 mL), and the organic layer was dried with anhydrous MgSO₄, and concentrated under vacuum. The crude product was purified by flash silica gel chromatography 40 g column, eluent of 0-20% ethyl acetate/Pet, ether gradient, to give benzyl but-3-yn-1-yl-L-prolinate. MS (ESI): m/z (M+H)⁺ 258. ¹H NMR (400 MHz, CDCl₃) δ 7.41-7.29 (m, 5H), 5.22-5.14 (m, 2H), 3.32 (dd, J=5.4, 8.8 Hz, 1H), 3.19 (dt, J=3.3, 8.1 Hz, 1H), 2.94 (td, J=7.8, 12.0 Hz, 1H), 2.72-2.64 (m, 1H), 2.51-2.44 (m, 1H), 2.39 (dt, J=2.6, 7.6 Hz, 2H), 2.18-2.0) (m, 1H), 2.01-1.89 (m, 3H). 1.87-1.78 (m, 1H).

### Step D: Benzyl (3S,8aS)-7-oxooctahydroindolizine-3-carboxylate (Int-AM4)

To a solution of benzyl but-3-yn-1-yl-L-prolinate (Int-AM3) (2.5 g, 9.72 mmol) and 4 A molecular sieve (5×weight of m-CPBA) in DCM (25 mL) was added m-CPBA (1.972 g, 9.72 mmol) under N₂ at 0° C. The reaction mixture was stirred at 0° C. for 20 min. Then Ph₃PAuNTf₂ (0.359 g, 0.486 mmol) was added into the reaction at −78° C. under N₂ atmosphere. The reaction mixture was stirred at −78° C. for 16 h. The reaction mixture was diluted with DCM (50 mL) and the molecular sieves were filtered off. The filtrate was washed with 5% aqueous Na₂CO₃, dried with Na₂SO₄, and concentrated under reduced pressure to give the residue which was purified by flash silica gel chromatography 24 g column, Eluent of 20% ethyl acetate in petroleum ether gradient, to give benzyl (3S,8aS)-7-oxooctahydroindolizine-3-carboxylate. MS (ESI): m/z (M+H)⁺ 274.

### Step E: Benzyl (3S,8aS)-7,7-difluorooctahydroindolizine-3-carboxylate (Int-AM5)

To a solution of benzyl (3S,8aS)-7-oxooctahydroindolizine-3-carboxylate (Int-AM4) (2 g, 7.32 mmol) in DCM (10 mL) was added 4-tert-butyl-2,6-dimethylphenylsulfur trifluoride (3.66 g, 14.63 mmol) and triethylamine trihydrofluoride (7.15 mL, 43.9 mmol) at 20° C. The reaction mixture was stirred at 50° C. for 2 h. Reaction was monitored using LC-MS, The reaction mixture was diluted with EtOAc (60 mL), washed with NaHCO₃(aq., 10 mL×2), brine (10 mL), dried over Na2SO₄ and concentrated in vacuo, the residue was purified by flash silica gel chromatography 24 g column, Eluent of 0-10% EtOAc/Pet, ether gradient to give benzyl (3S,8aS)-7,7-difluorooctahydroindolizine-3-carboxylate. MS (ESI): m/z (M+H)⁺ 296. ¹H NMR (400 MHz, CDCl₃) δ 7.41-7.31 (m, 5H), 5.15 (d, J=2.0 Hz, 2H), 3.84 (dd, J=3.1, 9.0 Hz, 11H), 3.25-3.14 (m, 1H), 3.00 (tdd, J=2.3, 5.0, 11.8 Hz, 1H), 2.83-2.73 (m, 1H), 2.29-2.15 (m, 2H), 2.14-2.04 (m, 1H), 2.03-1.96 (m, 2H), 1.94-1.82 (m, 1H), 1.59-1.43 (m, 2H).

### Step F: ((3S,8aS)-7.7-difluorooctahydroindolizin-3-yl)methanol (Int-AM6)

To a solution of benzyl (3S,8aS)-7,7-difluorooctahydroindolizine-3-carboxylate (Int-AM5) (200 mg, 0.677 mmol) in THF (5 mL) was added aluminum(III) lithium hydride (103 mg, 2.71 mmol) at 0° C., and the reaction mixture was stirred at 20° C. for 1 h. The reaction mixture was quenched with Na₂SO₄ (1 g), EtOAc (20 mL), H₂O (0.5 mL) and Na₂SO₄ (0.5 g) in sequence, and the resulting mixture was filtered. The filtrate was concentrated in vacuo, and the residue was purified by flash silica gel chromatography 4 g column, DCM/MeOH=10/1 to give ((3S, 8aS)-7,7-difluorooctahydroindolizin-3-yl)methanol. MS (EST): m/z (M+H)⁺ 191.

### Intermediate AN7: (3-(((tert-butyldimhenylsilyl)oxy)methyl)tetrahydro-1H-pyrrolizin-7a(5H₁-yl)methanol (Int-AN7-PK2)

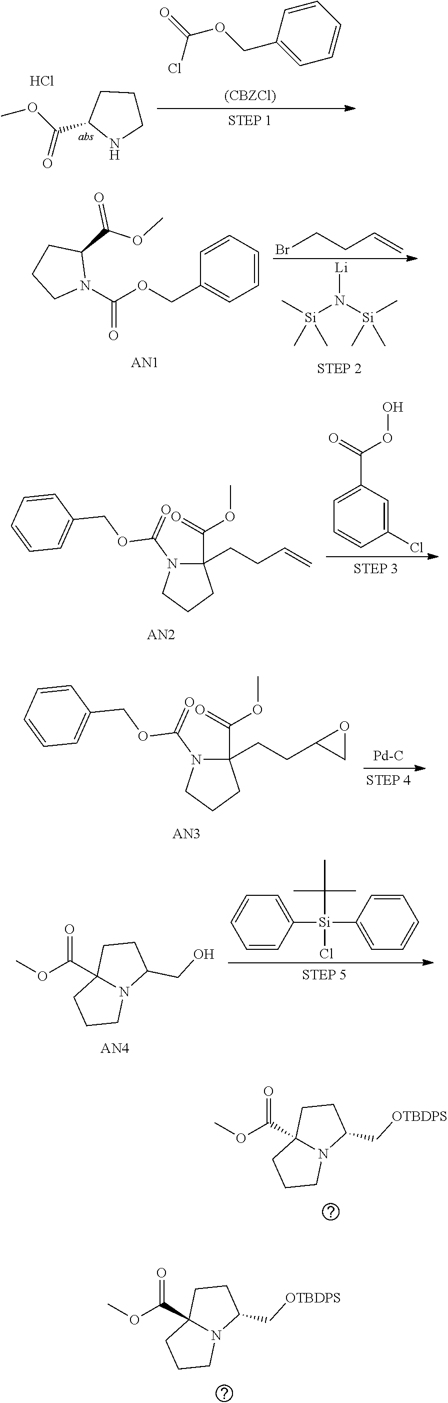

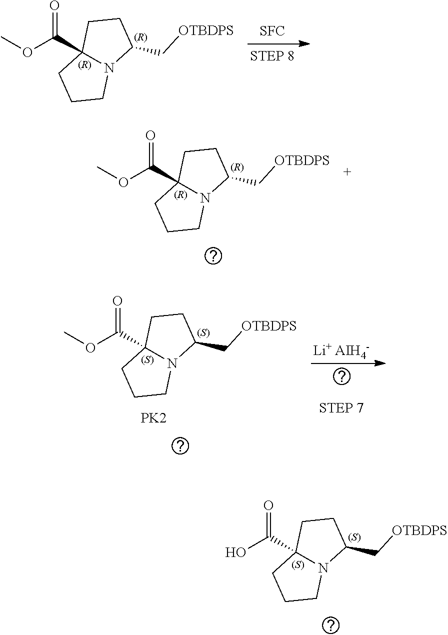

### STEP 1:®-benzyl2-methyl (S)-pyrrolidine-12-dicarboxylate (Int-AN1)

To a stirred solution of L-proline methyl ester hydrochloride (51.5 g, 311 mmol) and TEA (108 ml. 777 mmol) in DCM (400 ml) was added Cbz-Cl (62.1 m1H 435 mmol) at 0° C., and then the reaction mixture was stirred at 25° C. for 3 h. The mixture was diluted with water (300 mL) and extracted with EtOAc (250 mL×3), the organic layers were washed with sat. NaCl (400 mL) dried over Na₂SO₄ and concentrated. The residue was purified by flash silica gel chromatography Silica-CS(120 g), Eluent of 0-36% Ethyl acetate/Petroleum ether gradient to give crude product (78 g) as yellow oil. Then crude product was purified by reversed HPLC (Biotage; 1000 mg Agela, C18, 20-35 μm Eluent of 0%˜52% ACNH₂O (0.5% oTFA) gradient <<Li 30 mUJmin) to give 1-benzyl 2-methyl (S)-pyrrolidine-1.2-dicarboxylate. MS (ESI): m/z: (M+H)⁺ 264. ¹H NMR (400 MHz, CHLOROFORM-d) 6=7.41-7.28 (m, 5H), 5.26-5.01 (m, 2H), 4.48-4.32 (nm, 1H), 3.77-3.58 (m, 4H), 3.57-3.45(in, 1H), 2.33-2.13(in, 1H), 2.06-1.87(in, 3H).

### STEP 2: 1-STP 1benzyl 2-methyl 2-2-(but-3-en-1-yl)pyrolidine-1.2-dicarboxylate (Int-AN1

To a stirred solution of 1-benzyl 2-methyl (S)-pyrrolidine-1,2-dicarboxylate (Int-AN1) (4.5 g, 17.09 mmol) in THF (10 ml) was added LiHMDS (20.51 ml, 20.51 mmol) at −78° C. under N₂. The reaction was stirred at −78° C. for 1 h under N₂. Then 4-bromobut-1-ene (4.61 g, 34.2 mmol) was added under N2, the reaction mixture was stirred at 25° C. for 1 h under N2. To the reaction mixture was added NH₄Cl (40 mL) and the reaction mixture was extracted with EtOAc (20 mL×3), the organic layers were washed with sat. NaCl (40 mL) dried over Na₂SO₄ and concentrated. The residue was purified by flash silica gel chromatography Silica-CS(40 g), Eluent of 0-27% Ethyl acetate/Petroleum ether gradient to give 1-benzyl 2-methyl 2-(but-3-en-1-yl)pyrrolidine-1,2-dicarboxylate. MS (ESI): m/z (M+H)⁺ 318. ¹H NMR (400 MHz, METHANOL-d₄) δ=7.42-7.24 (m, 5H), 5.86-5.63 (m, 1H), 5.15-5.06 (m, 2H), 5.03-4.88 (m, 2H), 3.77-3.63 (m, 3H), 3.53-3.43 (m, 2H), 2.26-2.03 (m, 4H), 1.98-1.83 (m, 4H).

### STEP 3: 1-benzyl 2-methyl 2-(2-(oxiran-2-yl)ethyl)pyrrolidine-1,2-dicarboxylate (Int-AN3)

To a solution of 1-benzyl 2-methyl 2-(but-3-en-1-yl)pyrrolidine-1,2-dicarboxylate (Int-AN2) (3.75 g, 11.82 mmol) in DCM (50 ml) was added m-CPBA (6.37 g, 29.5 mmol)(80%), and the resulting mixture was stirred at 20° C. for 2 h. The reaction was filtered and the filtrate was washed with Na₂SO₃(aq., 30 mL) and brine. The organic layer was dried over Na₂SO₄, and purified by flash silica gel chromatography 40 g column, Eluent of 15% ethyl acetate in petroleum ether gradient to give 1-benzyl 2-methyl 2-(2-(oxiran-2-yl)ethyl)pyrrolidine-1,2-dicarboxylate. MS (ESI): m/z (M+H)⁺ 334, 1H NMR (500 MHz, CHLOROFORM-d) 5=7.41-7.28 (m, 5H), 5.21-5.03 (m, 2H), 3.84-3.72 (m, 1H), 3.70 (d, J=4.7 Hz, 2H), 3.53-3.43 (m, 2H), 2.96-2.76 (m, 1H), 2.74-2.65 (m, 1H), 2.53-2.22 (n. 2H). 2.16-2.05 (m, 2H), 2.05-1.98 (m, 1H). 1.97-1.88 (m, 1H), 1.88-1.78 (m, 1H), 1.59-1,39 (m, 2H).

### STEP 4: Methyl 3-(hydroxymethyl)tetrahydro-1H-pyrrolizine-7a(5H)-carboxylate (Int-AN4)

To a solution of 1-benzyl 2-methyl 2-(2-(oxiran-2-yl)ethyl)pyrrolidine-1,2-dicarboxylate (Int-AN3) (3.7 g, 11.10 mmol) in MeOH (50 ml) was added Pd-C(0.591 g, 0.555 mmol)(10%), the resulting mixture was stirred at 20° C. for 2 h. The reaction mixture was filtered and washed with MeOH(50 mL), the filtrate was concentrated to afford methyl 3-(hydroxymethyl)tetrahydro-1H-pyrrolizine-7a(5H)-carboxylate, which was used directly in the next step without purification. MS (ESI): m/z (M+H)⁺ 200.

### STEP 5: Methyl 3-(((tert-butyldiphenylsilyl)oxy)methyl)tetrahydro-1H-pyrrolizine-7a(5H)-carboxylate (Int-AN5-PKI and Int-AN5-PK2)

To a solution of methyl 3-(hydroxymethyl)tetrahydro-1H-pyrrolizine-7a(5H)-carboxylate (Int-AN4) (2.0 g, 10.04 mmol) in DCM (30 ml) was added imidazole (1,367 g, 20.08 mmol) and TBDPS-C₁ (3.09 ml, 12.05 mmol), the resulting mixture was stirred at 20° C. for 1 h. The reaction mixture was filtered and the filtrate was concentrated, the residue was purified by flash silica gel chromatography 40 g column, Eluent of 8% ethyl acetate in petroleum ether gradient to give methyl (3R,7aS)-3-(((tert-butyldiphenylsilyl)oxy)methyl)tetrahydro-1H-pyrrolizine-7a(5H)-carboxylate (P01, the first eluting isomer, cis) and methyl (3R,7aR)-3-(((tert-butyldiphenylsilyl)oxy)methyl)tetrahydro-1H-pyrrolizine-7a(5H)-carboxylate (P02, the second eluting isomer, trans. MS (ESI): m/z (M+H)⁺ 438. Int-AN5-PK1-Cis ¹H NMR (500 MHz, CHLOROFORM-d) δ=7.77-7.68 (m, 4H), 7.46-7.34 (m, 6H), 3.81-3.73 (m, 1H), 3.64 (s, 3H), 3.55 (dd, J=7.4, 9.8 Hz, 1H), 3.04 (td, J=6.6, 10.9 Hz, 1H), 2.91-2.82 (m, 1H), 2.73-2.66 (m, 1H), 2.38 (ddd, J=2.4, 6.5, 12.4 Hz, 1H), 2.22-2.15 (m, 1H), 2.05-1.99 (m, 1H), 1.86-1.76 (m, 2H), 1.75-1.69 (m, 2H), 1.63-1.54 (m, 1H), 1.07 (s, 9H). Int-AN5-PK2 Trans ¹H NMR (500 MHz, CHLOROFORM-d) 6=7.59-7.51 (m, 4H), 7.34-7.22 (m, 6H), 3.85 (dd, J=4.4, 10.5 Hz, 1H), 3.68 (dd, J=6.4, 10.4 Hz, 1H), 3.63-3.56 (m, 3H), 3.30-3.20 (m, 1H), 2.82-2.76 (m, 1H), 2.73-2.64 (m, 1H), 2.34 (ddd, J=4.0, 5.8, 12.6 Hz, 1H), 2.13-2.03 (m, 1H), 1.87-1.79 (m, 1H), 1.75-1.62 (m. 4H), 1.52-1.43 (m, 1H), 0.93 (s, 9H).

### STEP 6: Methyl (3R,7aR)-3-(((tert-butyldiphenylsilyl)oxy)methyl)tetrahydro-1H-pyrrolizine-7a(5H)-carboxylate and Methyl (3S,7aS)-3-(((tert-butyldiphenylsilyl)oxy)methyl)tetrahydro-1H-pyrrolizine-7a(5H)-carboxyalate (Int-AN6-PK1 and Int-AN6-PK2)

The mixture of methyl 3-(((tert-butyldiphenylsilyl)oxy)methyl)tetrahydro-1H-pyrrolizine-7a(5H)-carboxylate (Int AN5-PK2) (11.5 g, 26.3 mmol) was separated by preparative SFC (Column REG1S (s,s) WHELK-O1 (250 mm×50 mm, 10 um): Condition 0.1% NH₃H2O ETOH, Begin B 20%, End B 20%: Gradient Time(min) 100% B Hold Time(min); FlowRate(ml/min) 200) to afford methyl (3R,7aR)-3-(((tert-butyldiphenylsilyl)oxy)methyl)tetrahydro-1H-pyrrolizine-7a(5H)-carboxylate (the first eluting isomer, >99.9% ee value, AN6-PK1) as light red solid and methyl (3S,7aS)-3-(((tert-butyldiphenylsilyl)oxy)methyl)tetrahydro-1H-pyrrolizine-7a(5H)-carboxylate (the second eluting isomer, >95.018% ee value, AN6-PK2) as light red oil. MS (ESI), m/z (M+H)⁺ 438.

### STEP 7: ((3S,7aS)-3-(((tert-butyl)diphenylsilyl)oxy)methyl)tetrahydro-1H-pyrrolizin-7a(5H)-yl)methanol (Int-AN7-PK2)

To a solution of methyl methyl (3S,7aS)-3-(((tert-butyldiphenylsilyl)oxy)methyl)tetrahydro-1H-pyrrolizine-7a(5H)-carboxylate (Int-AN6-PK2) (4 g, 9.14 mmol) in THF (40 mL) was added aluminum(III) lithium hydride (0.451 g, 11.88 mmol) at 0° C., and the solution was stirred at 25° C. for 1 hours. The reaction mixture was extracted with EtOAc(30 ml), H₂O(3.5 ml) and Na₂SO₄(10 g), and the resulting mixture was filtered. The filtrate was concentrated in vacuo, and the residue was purified by flash silica gel chromatography 40 g column, DCM/MeOH=10/1 to give ((3S,7aS)-3-(((tert-butyldiphenylsilyl)oxy)methyl)tetrahydro-1H-pyrrolizin-7a(5H)-yl)methanol. MS (ESI): m/z (M+H)⁺ 410. ¹H NMR (400 MHz, CHLOROFORM-d) δ=7.61-7.47 (m, 4H), 7.46-7.30 (m, 6H), 4.05 (dd, J=2.6, 12.8 Hz, 1H), 3.88-3.74 (m, 2H), 3.68-3.51 (m, 3H), 3.38-3.25 (m, 1H), 2.20-1.94 (m, 5H), 1.89-1.79 (m, 1H), 1.79-1.72 (m, 1H), 1.72-1.62 (m, 1H), 1.02 (s, 9H).

### Intermediate A04: 7-(8-ethynyl-7-fluoronaphthalen-1-yl)-6A-difluoro-2-(((3S,7aS)-3-(fluoromethyl)tetrahydro-1H-pyrrolizin-7a(5H)-yl)methoxy)quinazolin-4-ol (Int-A04)

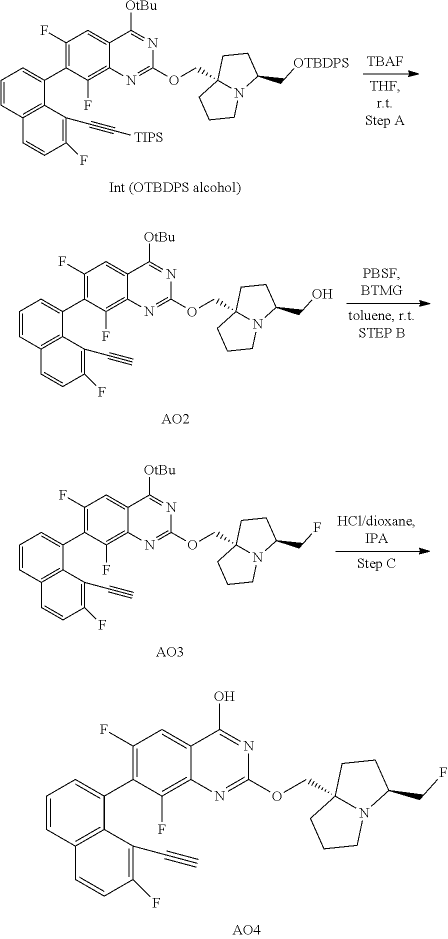

### Step A: ((3S,7aS)-7a-(((4-(tert-butoxy)-7-(8-ethynyl-7-fluoronaphthalen-1-yl)-6,8-difluoroquinazolin-2-yl)oxy)methyl)hexahydro-1H-pyrrolizin-3-yl)methanol (Int-A02)

To a solution of 4-(tert-butoxy)-2-(((3S,7aS)-3-(((tert-butyldiphenylsilyl)oxy)methyl)tetrahydro-1H-pyrrolizin-7a(5H)-yl)methoxy)-6,8-difluoro-7-(7-fluoro-8-((triisopropylsilyl)ethynyl)naphthalen-1-yl)quinazoline {(prepared from protected alcohol (Int-AN7-PK2), described in sections above} (500 mg, 0.515 mmol) in THF (5 mL) was added TBAF (1.082 mL. 1.082 mmol, 1 M in THF) at 25° C. The mixture was stirred at 25° C. for 15 h. The mixture was diluted with DCM (20 mL), washed with H₂O (5 mL), dried over Na₂SO₄, filtered and the solvent was evaporated under reduced pressure. The crude product was purified by flash silica gel chromatography 12 g column, Eluent of 5% MeOH in DCM (basified with TEA) gradient to give ((3S,7aS)-7a-(((4-(tert-butoxy)-7-(8-ethynyl-7-fluoronaphthalen-1-yl)-6,8-difluoroquinazolin-2-yl)oxy)methyl)hexahydro-1H-pyrrolizin-3-yl)methanol. MS (ESI): m/z (M+H)⁺ 576.

### Step B: 4-(tert-butoxy)-7-(8-ethynyl-7-fluoronaphthalen-1-yl)-6,8-difluoro-2-(((3S,7aS)-3-(fluoromethyl)tetrahydro-1H-pyrrolizin-7a(5H)-yl)methoxy)quinazoline (Int-AO3)

To a solution of ((3S,7aS)-7a-(((4-(tert-butoxy)-7-(8-ethynyl-7-fluoronaphthalen-1-yl)-6,8-difluoroquinazolin-2-yl)oxy)methyl)hexahydro-1H-pyrrolizin-3-yl)methanol (Int-AO2)(100 mg, 0.174 mmol) in toluene (0.4 mL) was added 2-(tert-butyl)-1,1,3,3-tetramethylguanidine (BTMG) (89.5 mg, 0.521 mmol) and 1.1,2,2,3,3,4,4,4-nonafluorobutane-1-sulfonyl fluoride (PBSF) (78.5 mg, 0.261 mmol) at 20° C. The mixture was stirred at 20° C. for 1.5 h. 2-(tert-butyl)-1,1,3,3-tetramethylguanidine (89.5 mg, 0.521 mmol) and 1.1,2,2,3,3,4,4,4-nonafluorobutane-1-sulfonyl fluoride (78.5 mg, 0.261 mmol) was added to the reaction solution, then the mixture was stirred at 20° C. for another 15 min. The mixture was quenched with H₂O (1 mL), and extracted with EtOAc (2×2 mL). The organic layer was dried over Na₂SO₄, filtered and the solvent was evaporated under reduced pressure to give the crude product 4-(tert-butoxy)-7-(8-ethynyl-7-fluoronaphthalen-1-yl)-6,8-difluoro-2-(((3S,7aS)-3-(fluoromethyl)tetrahydro-1H-pyrrolizin-7a(5H)-yl)methoxy)quinazoline which was used in the next step directly without further purification. MS (ESI): m/z (M+H)⁺ 578.

### Step C: 7-(8-ethynyl-7-fluoronaphthalen-1-yl)-6,8-difluoro-2-(((3S,7aS)-3-(fluoromethyl)tetrahydro-1H-pyrrolizin-7a(5H)-yl)methoxy)quinazolin-4-ol (Int-AO4)

A solution of 4-(tert-butoxy)-7-(8-ethynyl-7-fluoronaphthalen-1-yl)-6,8-difluoro-2-(((3S,7aS)-3-(fluoromethyl)tetrahydro-1H-pyrrolizin-7a(5H)-yl)methoxy)quinazoline (Int-AO3)(99 mg, 0.173 mmol) in HCl (2 mL, 4 M in dioxane) and IPA (2 mL) was stirred at 25° C. for 1 h. LCMS showed starting material was consumed and desired peak was formed. The mixture was evaporated under reduced pressure, and the residue was dissolved in MeCN (1 mL), then the solution was basified with saturated aqueous sodium bicarbonate until pH 7. The solution was purified by reverse preparative HPLC (Column: Boston Prime C18 150×30 mm×5 um: Condition: water (0.1% FA)-ACN, to give 7-(8-ethynyl-7-fluoronaphthalen-1-yl)-6,8-difluoro-2-(((3S,7aS)-3-(fluoromethyl)tetrahydro-1H-pyrrolizin-7a(5H)-yl)methoxy)quinazolin-4-ol, Formic Acid. MS (ESI): m/z (M+H)⁺ 522.

Int-AO4 was used for the synthesis of Ex, following similar steps described in other sections.

### Intermediate AP4: 4-bromo-3A-dimethyl-1-(tetrahydro-2H-uyran-2-yl)-5-(tifluoromethyl)-1H-indazole (Int-AP4)

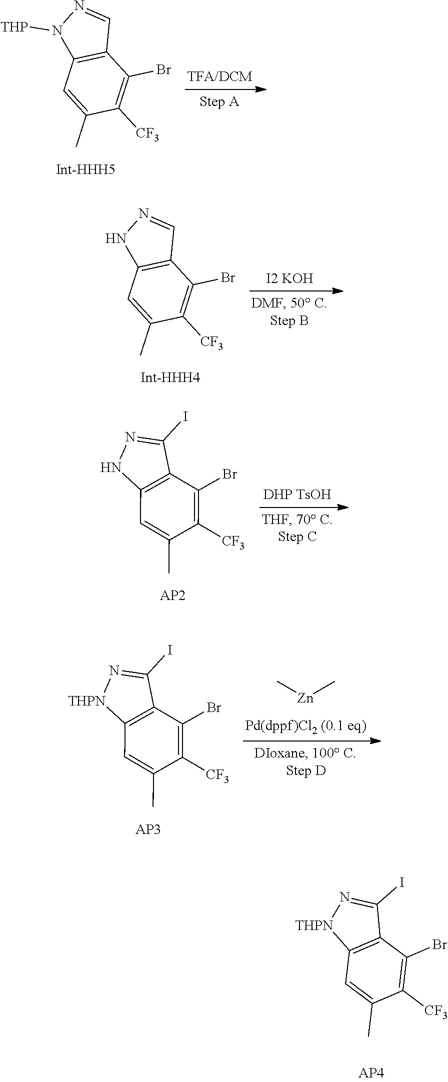

### Step A: 4-bromo-6-methyl-5-(trifluoromethyl)-1H-indazole (Int-HHH4)

To a solution of 4-bromo-6-methyl-1-(tetrahydro-2H-pyran-2-yl)-5-(trifluoromethyl)-1H-indazole (Int-HHH5) (2 g, 5.51 mmol) in DCM (15 mL) was added TFA (4.24 mL, 55.1 mmol). The mixture was heated to 25° C. for 15 h. Upon cooling to room temperature, the mixture was concentrated under reduced pressure. The crude was purified by silica gel chromatography column Silica-CS -20 g, Eluent of 0-60% Ethyl acetate/Petroleum ether gradient@ 30 mL/min) to give 4-bromo-6-methyl-5-(trifluoromethyl)-1H-indazole. MS (ESI) [M+H]⁺ m/z: 279, 281.

### Step B: 4-bromo-3-iodo-6-methyl-5-(trifluoromethyl)-1H-indazole (Int-AP2)

To a solution of 4-bromo-6-methyl-5-(trifluoromethyl)-1H-indazole (Int-AP1) (1.5 g, 5.38 mmol) in DMF (15 mL) was added KOH (0.905 g, 16.13 mmol), then followed by I₂ (2.73 g, 10.75 mmol) at 20° C. The mixture was heated to 50° C. for 15 h. Upon cooling to room temperature, the mixture was diluted with ethyl acetate (5 mL×3) and water. The mixture was concentrated under reduced pressure. The crude was purified by flash silica gel chromatography (Column Silica-CS-20 g, Eluent 0-50% Ethyl acetate/Petroleum ether gradient®<L 30 mL/min) to give 4-bromo-3-iodo-6-methyl-5-(trifluoromethyl)-1H-indazole. MS (ESI) [M+H]1 m/z: 404, 406.

### Step C: 4-bromo-3-iodo-6-methyl-1-(tetrahydro-2H-pyran-2-yl)-5-(trifluoromethyl)-1H-indazole (Int-AP3)

To a solution of 4-bromo-3-iodo-6-methyl-5-(trifluoromethyl)-1H-indazole (Int-AP2) (1.2 g, 2.96 mmol) in DMF (15 mL) was added TsOH (0.282 g, 1.482 mmol), then followed by 3.4-dihydro-2h-pyran (DHP) (0.542 mL, 5.93 mmol) at 20° C. The mixture was heated to 70° C. for 15 h. Upon cooling to room temperature, the mixture was diluted with ethyl acetate (5 mL×3) and water. The organic layer was separated, dried over magnesium sulfate, filtered, and concentrated under reduced pressure. The mixture was filtered and the solids were found to be the desired product. The mixture was purified by flash silica gel chromatography 12 g, Eluent of 0-20% Ethyl acetate/Petroleum ether gradient d 30 mL/min) to give 4-bromo-3-iodo-6-methyl-1-(tetrahydro-2H-pyran-2-yl)-5-(trifluoromethyl)-1H-indazole. MS (ESI) [M+H]⁺ m/z: 489, 491.

### Step D: 4-bromo-3,6-dimethyl-1-(tetrahydro-2H-pyran-2-yl)-5-(trifluoromethyl)-1H-indazole (Int-AP4)

To a solution of 4-bromo-3-iodo-6-methyl-1-(tetrahydro-2H-pyran-2-yl)-5-(trifluoromethyl)-1H-indazole (Int-AP3) (500 mg, 1.022 mmol) in dioxane (10 mL) was added PdCl₂(dppf) (112 mg, 0.153 mmol), then followed by dimethylzinc (0.511 mL, 1.022 mmol, 2N) at 100° for 1 h. LCMS showed the reaction was complete. Water (5 mL) was added, the organic layer was separated and the aqueous layer was re-extracted with EtOAc (15 mL×3). The combined organic layers were washed with brine (10 mL), dried over anhydrous Na₂SO₄, filtered and concentrated. The residue was purified by reverse preparative HPLC (Column: Phenomenex Synergi C18 150×30 mm×4 um; Condition: water(0.1% TFA)-ACN; Begin B--End B: 22--42; Gradient Time (min): 10; 100% B Hold Time (min): 1: FlowRate (mL/min): 25) to give 4-bromo-3,6-dimethyl-1-(tetrahydro-2H-pyran-2-yl)-5-(trifluoromethyl)-1H-indazole. MS (ESI) [M+H]⁺ m/z:377, 379. ¹H NMR (400 MHz, METHANOL-d₄) δ 7.56 (s, 1H), 5.71 (dd, J=2.7, 9.8 Hz, 1H), 3.98 (br d, J=1.1.3 Hz, 1H), 3.79 (dt, J=3.1, 11.0 Hz, 1H), 2.76 (s, 3H), 2.65-2.60 (m, 3H), 2.50-2.40 (m, 1H), 2.16-2.08 (m, 1H), 1.98 (br dd, J=3.5, 13.3 Hz, 1H), 1.84-1.63 (m, 3H).

### Intermediate AQ5: 1-bromo-3-chloro-5-(methoxymethoxy)-2-(trifluoro methoxy)benzene (Int-AO5)

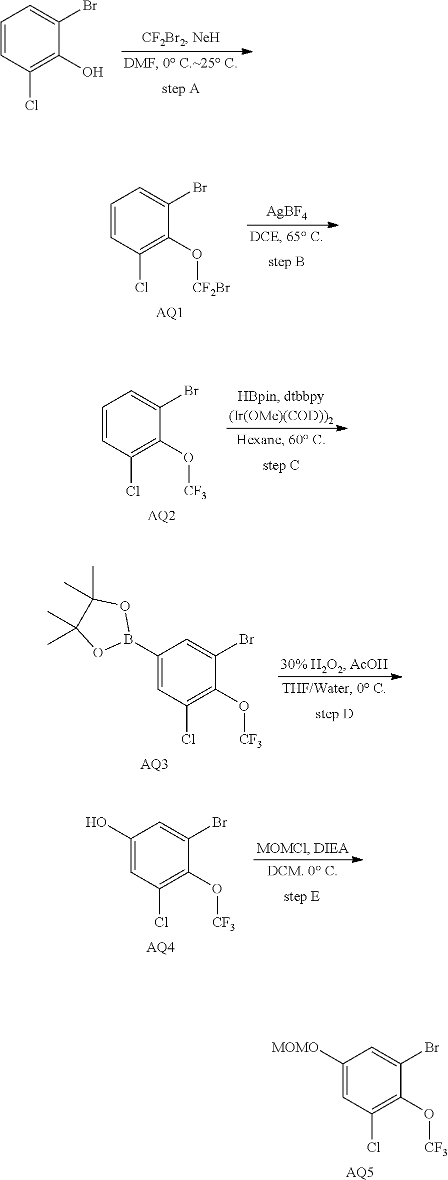

### Step A: 1-bromo-2-(bromodifluoromethoxy)-3-chlorobenzene (Int-AOI)

To a solution of 2-bromo-6-chlorophenol (7.5 g, 36.2 mmol) in DMF (150 mL) was added NaH (3.18 g, 80 mmol) (60% in mineral oil) in portions at 0° C. for 5 min. dibromodifluoromethane (9.10 mL, 108 mmol) was added at 0° C., and the mixture was stirred at 25° C. for 16 h. The reaction mixture was quenched with brine (200 mL) and saturated NH₄Cl aqueous solution (200 mL) at 0° C. The aqueous layer was extracted with EtOAc (50 mL×2). The combined organic layers were dried over Na₂SO₄ and filtered. The filtrate was concentrated under reduced pressure to give the residue which was purified by flash silica gel chromatography 20 g column, Eluent of 0% Pet, ether to give 1-bromo-2-(bromodifluoromethoxy)-3-chlorobenzene. ¹H NMR (400 MHz, CDCl₃) δ 7.81 (dd, J=1.4, 8.1 Hz. 1H), 7.71 (dd, J=1.4, 8.1 Hz, 1H), 7.38 (t, J=8.1 Hz, 1H). ¹⁹F NMR (400 MHz. DMSO-d₆) δ-12.57 ppm.

### Step B: 1-bromo-3-chloro-2-(trifluoromethoxy)benzene (Int-AO2)

To a solution of 1-bromo-2-(bromodifluoromethoxy)-3-chlorobenzene (Int-AQ1) (7.5 g, 22.30 mmol) in DCE (150 mL) was added silyler tetrafluoroborate (15.19 g, 78 mmol) below 15° C. under N₂ atmosphere. The mixture was stirred at 65° C. for 3 h. The reaction mixture was poured into ice water (70 mL), and diluted with DCM (70 mL). The mixture was filtered through a pad of CELITE. The organic laver was separated, and the aqueous layer was extracted with DCM (70 mL×2). The combined organic layers were dried over anhydrous Na₂SO₄, filtered and concentrated under reduced pressure. The residue was purified by flash silica gel chromatography 80 g column. Eluent of 0% Pet, ether to give 1-bromo-3-chloro-2-(trifluoromethoxy)benzene. ¹H NMR (400 MHz, CDCl₃) δ 7.83 (dd, J=1.4, 8.1 Hz, 1H). 7.74 (dd, J=1.4, 8.1 Hz, 1H), 7.40 (t, J=8.2 Hz, 1H). ¹⁹F NMR (400 MHz, DMSO-c) S -54.63 ppm.

### Step: 2-(3-bromo-5-chloro-4-(trifluoromethoxy)henyl-4,4,5,5-tetramethyl-1,3,2-dioxaborolane (Int-AQ3)

To a mixture of 1-bromo-3-chloro-2-(trifluoromethoxy)benzene (IntAQ2) (4.8 g, 17.43 mmol) in hexane (10 mL) was added 4,4′-di-tert-butyl-2,2′-bipyridine (0.281 g, 1.046 mmol) and bis(1,5-cyclooctadiene)dimethoxydiiridium (0.578 g, 0.871 mmol) and pinacolborane (0.316 ml, 2.178 mmol) (13.3g, 105 mmol). The reaction mixture was stirred at 60° C. for 1 h under N₂ atm. The reaction mixture was concentrated under reduced pressure to give crude 2-(3-bromo-5-chloro-4-(trifluoromethoxy)phenyl)-4,4,5,5-tetramethyl-1,3,2-dioxaborolane.

### Step D: 3-bromo-5-chloro-4-(trifluoromethoxy)phenol (Int-AO4)

To a solution of 2-(3-bromo-5-chloro-4-(trifluoromethoxy)phenyl)-4,4,5,5-tetramethyl-1,3,2-dioxaborolane (Int-AQ3) (7 g, 17.44 mmol) in THF (40 mL) and water (20 mL) was added acetic acid (64.0 mL, 1120 mmol) and H₂O₂ (34.0 mL, 333 mmol) (30% in water) at 0° C. The mixture was stirred at 0° C. for 1 h. The reaction mixture was quenched with saturated sodium sulfite solution (60 mL) and EtOAc (60 mL), and separated. The aqueous layer was extracted with EtOAc (60 mL×2). The combined organic layers were dried over Na₂SO₄ and filtered. The filtrate was concentrated under reduced pressure to give the residue which was purified by flash silica gel chromatography 80 g column, Eluent of 15% EtOAc/Pet, ether to give 3-bromo-5-chloro-4-(trifluoromethoxy)phenol. ¹H NMR (400 MHz, CDCl₃) δ 7.05 (d, J=2.9 Hz, 1H), 6.93 (d, J=2.9 Hz, 1H), 6.58 (br s. 1H).

### Step E: 1-bromo-3-chloro-5-(methoxymethoxy)-2-(trifluoromethoxy)benzene (Int-A05)

To a solution of 3-bromo-5-chloro-4-(trifluoromethoxy)phenol (Int-AQ4) (1 g, 3.43 mmol) in DCM (15 mL) was added DIEA (3.00 mL, 17.16 mmol) and MOMCl (1g, 0.943 mL, 12.42 mmol) at 0° C., and the mixture was stirred at 0° C. for 10 min. The reaction mixture was diluted with DCM (30 mL), and washed with brine (10 mL×2). The organic layer was concentrated in vacuo, and the residue was purified by flash silica gel chromatography 12 g column, Pet, ether/EtOAc=20/1 to give 1-bromo-3-chloro-5-(methoxymethoxy)-2-(trifluoromethoxy)benzene. ¹H NMR (400 MHz, CDCl₃) δ 7.22 (br s, 1H), 7.10 (br s, 1H), 5.13 (br s, 2H), 3.46 (br d, 1-2.4 Hz, 3H).

### Intermediate AR3: 3-bromo-2,5-difluoro-N,N-bis(4-methoxybenzyl)-4-(trifluoromethyl)aniline (Int-AR3)

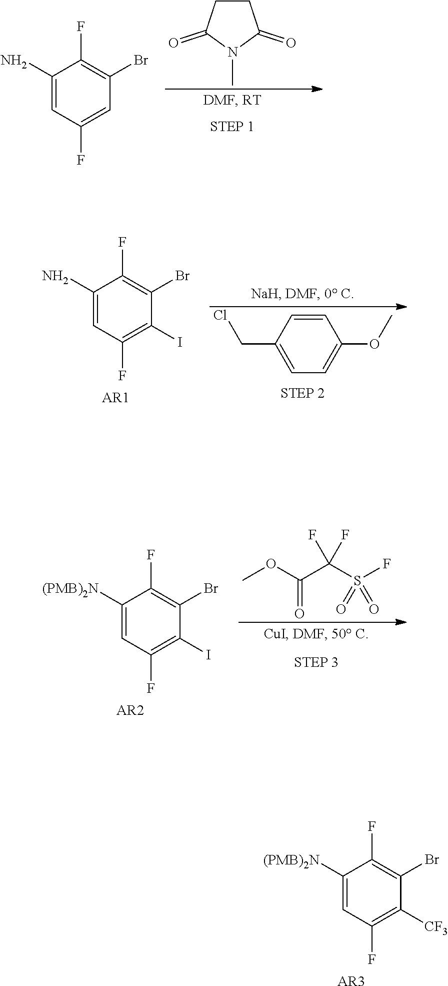

### STEP 1: 3-bromo-2,5-difluoro-4-iodoaniline (Int-AR1)

To a solution of 3-bromo-2,5-difluoroaniline in DMF (11 ml) was added 1-iodopyrrolidine-2,5-dione (368 mg, 1.636 mmol). The reaction mixture was stirred at RT for 4 hr and then diluted with EtOAc, washed with water, and brine solution and concentrated under reduced pressure to give 3-bromo-2,5-difluoro-4-iodoaniline. MS (ESI): m/z (M+H)⁺ 333.

### STEP 2: 3-bromo-2,5-difluoro-4-iodo-N,N-bis(4-methoxybenzyl)aniline (Int-AR2)

To a solution of 3-bromo-2,5-difluoro-4-iodoaniline (int-ARl) (350 mg, 1.048 mmol) in DMF (5 ml) was added sodium hydride (126 mg, 3.14 mmol) under nitrogen at 0° C. The reaction mixture was stirred at the same temperature for 1 hr, and then added 1-(chloromethyl)-4-methoxybenzene (0.285 ml, 2.096 mmol) and stirred for 1 hr. The reaction was quenched with sat. NH₄Cl, extracted with EtOAc. The combined extracts were washed with water and brine solution and then concentrated under reduced pressure. The product was purified by 0-15% of EtOAc in hexane to give 3-bromo-2,5-difluoro-4-iodo-N,N-bis(4-methoxybenzyl)aniline MS (ESI): m/z (M+H)⁺ 574.

### STEP 3: 3-bromo-2,5-difluoro-N,N-bis(4-methoxybenzyl)-4-(trifluoromethyl) aniline (Int-AR3)

To a solution of 3-bromo-2,5-difluoro-4-iodo-N,N-bis(4-methoxybenzyl)aniline (Int-AR2) (350 mg, 0.610 mmol) in DMF (800 μL) was added Copper(l) iodide (290 mg. 1.524 mmol) followed by methyl 2,2-difluoro-2-(fluorosulfonyl)acetate (101 μL, 0.792 mmol). The reaction was heated to 50° C. for 2 hrs and then further heated at 100° C. for 3 hrs. The reaction was quenched with sat. NH₄Cl, solution, extracted with EtOAc. The combined extracts were washed with water and brine solution and then concentrated under reduced pressure. The residue was purified on 24 g silica gel column with 0-20% of EtOAc in hexane to give 3-bromo-2,5-difluoro-N,N-bis(4-methoxybenzyl)-4-(trifluoromethyl)aniline MS (ESI): m/z (M+H)⁺ 516.

### Intermediate AS3: 4-chloro-N,N-bis(4-methoxybenzyl)-6-methyl-5-(trifluoromethyl)pyrimidin-2-amine (Int-AS3)

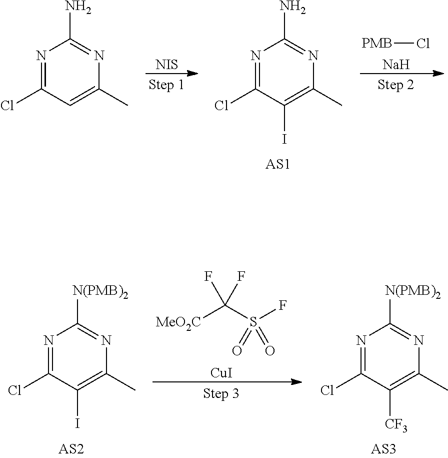

### Step 1: 4-chloro-5-iodo-6-methylpyrimidin-2-amine (Int-AS1)

2-Amino-4-chloro-6-methylpyrimidine (5 g, 34.8 mmol) was taken up in acetic acid (34.8 ml) and cooled to 0° C. N-iodosuccinimide (7.84 g, 34.8 mmol) was added and the reaction mixture was stirred at RT overnight. Reaction mixture was poured into mixture of ice water, sat. NaHCO₃, and sat. Na₂S203. The solid precipitant was filtered and washed with sat. NaHCO₃ and water. The solid was collected and dried in a vacuum oven to afford 4-chloro-5-iodo-6-methylpyrimidin-2-amine. [M+H]⁺ Found: 270.

### Step 2: 4-chloro-5-iodo-N,N-bis(4-methoxybenzyl)-6-methylpyrimidin-2-amine (Int-AS2)

Sodium hydride (1.781 g, 44.5 mmol) was taken up in DMF (44.5 ml) and cooled to 0° C. 4-Chloro-5-iodo-6-methylpyrimidin-2-amine (Int-AS1) (3 g, 11.13 mmol) was added portion-wise and the reaction mixture was stirred for 5 min. 4-Methoxybenzyl chloride (6.04 ml, 44.5 mmol) was added dropwise and the reaction mixture was stirred at 0° C. for 15 min, then rt for 15 min. The reaction was cooled to 0° C. and quenched with water, extracted with EtOAc (3x), washed with brine, dried over Na₂SO₄, and concentrated under reduced pressure. The residue was purified by column chromatography (0-50% EtOAc in hex) to afford 4-chloro-5-iodo-N,N-bis(4-methoxybenzyl)-6-methylpyrimidin-2-amine. [M+H]⁺ Found: 510.

### Step 3: 4-chloro-N,N-bis(4-methoxybenzyl)-6-methyl-5-(trifluoromethyl)pyrimidin-2-amine (Int-AS3)

4-Chloro-5-iodo-N,N-bis(4-methoxybenzyl)-6-methylpyrimdim-2-amine (Int-AS2) (2 g, 3.92 mmol), copper(I) iodide (2.242 g, 11.77 mmol), and methyl 2,2-difluoro-2-(fluorosulfonyl)acetate (1.507 g, 7.85 mmol) were added to vial and taken up in DMF (19.62 ml). The reaction mixture was heated to 100° C. and stirred overnight. The reaction mixture was filtered through CELITE, washed with EtOAc, washed with brine, dried over Na₂SO₄ and concentrated under reduced pressure. The residue was purified by silica gel column chromatography (0-50% EtOAc in hex) to afford 4-chloro-N,N-bis(4-methoxybenzyl)-6-methyl-5-(trifluoromethyl)pyrimidin-2-amine. [M+H]⁺ Found: 452.

### Intermediate AT3: 4,5-dichloro-N-(4-methoxybenzyl)quinolin-2-amine (Int AT3)

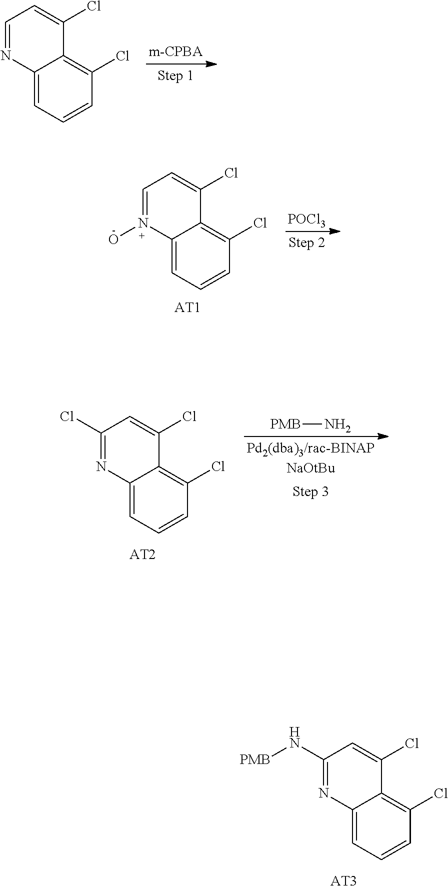

### Step 1: 45-dichloroumoline 1-oxide (Int-ATI)

To a 0° C. cooled solution of 4,5-dichloroquinoline (500 mg, 2.52 mmol) in DCM (5049 μl) was added m-CPBA (1162 mg, 5.05 mmol). The resultant mixture was allowed to stir at RT for 18h. The reaction mixture was diluted with DCM, washed with 3N aq. NaOH, and the aqueous layer was extracted with DCM. The combined organic layers were then washed with brine, dried over magnesium sulfate, and concentrated under reduced pressure to afford 4,5-dichloroquinoline 1-oxide. [M+H]⁺+ Found: 214.

### Step 2: 2,4,5-trichloroquinoline (Int-AT2)

To a 0° C. cooled solution of 4,5-dichloroquinoline 1-oxide (Int-AT1) (289 mg, 1.350 mmol) and TEA (0.226 ml, 1.620 mmol) in DCM (6 ml) was added a solution of POCl₃ (0.151 ml, 1.620 mmol) in DCM (2 ml), dropwise, via syringe. The resultant mixture was allowed to warm to RT and stir for 30 minutes, then was allowed to stir at 40° C. for 1h. The reaction mixture was quenched with water, neutralized with 2M aq. NaOH, and then extracted with DCM. The combined organic layers were then washed with brine, dried over magnesium sulfate, and concentrated under reduced pressure. The residue was purified by flash chromatography over silica (0-10% EtOAc:hexanes) to afford 2,4,5-trichloroquinoline. [M+H]⁺ Found: 232.

### Step 3: 4,5-dichloro-N-(4-methoxybenzyl)quinolin-2-amine (Int-AT3)

A solution of 2,4,5-trichloroquinoline (Int-AT2) (739 mg, 3.18 mmol), 4-methoxybenzylamine (PMB-NH₂) (0.457 ml, 3.50 mmol), Pd₂(dba)₃ (36.6 mg, 0.064 mmol), (R)-(+)-2,2′-bis(diphenylphosphino)-1,1′-binaphthyl (rac-BINAP) (79 mg, 0.127 mmol), and sodium tert-butoxide (916 mg, 9.54 mmol) in toluene (15 ml) in a vial was sealed, purged with nitrogen, and then allowed to stir at 80° C. for 1 h. The reaction mixture was quenched with sat. aqueous NH₄Cl and water, then extracted with EtOAc. The combined organic layers were then washed with brine, dried over magnesium sulfate, and concentrated under reduced pressure. The residue was purified by flash chromatography over silica (0-20% EtOAc:hexanes) to afford 4,5-dichloro-N-(4-methoxybenzyl)quinolin-2-amine. [M+H]⁺ Found: 333.

### Intermediate AU1: 6-chloro-N,N-bis(4-methoxybenzyl)-4-methyl-5-(2,2,2-trifluoroethyl)pyridin-2-amine (Int-AU1)

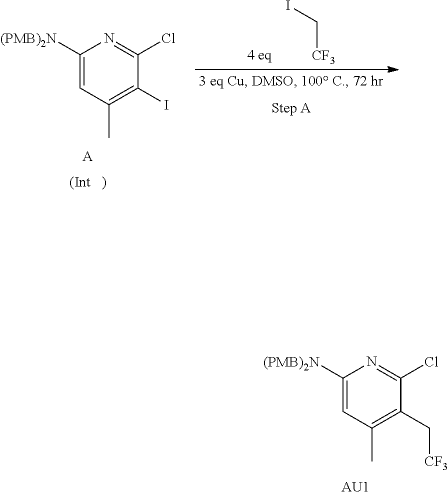

### Step A: 6-chloro-N,N-bis(4-methoxybenzyl-4-methyl-5-(2,2,2-trifluoroethyl)pyridin-2-amine (Int-AU)

A solution of 6-chloro-5-iodo-N,N-bis(4-methoxybenzyl)-4-methylpyridin-2-amine (Int-236P) (1 g, 1.965 mmol), 1.1,1-trifluoro-2-iodoethane (0.775 mL, 7.86 mmol), copper dust (0.375 g, 5.90 mmol) in anhydrous DMSO (4 mL) was stirred at 100° C. for 72 h under N₂ atmosphere. The reaction was purified by Prep-HPLC directly (phenomenex Gemini-NX C18 150×40 mm×5 um. Condition water (0.05% NH₃H?O+10 mM NH₄HCO3)-ACN to give 6-chloro-N,N-bis(4-methoxybenzyl)-4-methyl-5-(2,2,2-trifluoroethyl)pyridin-2-amine. MS (ESI): m/z (M+H)⁺ 465. ¹H NMR (400 MHz, DMSO-d( ) δ 7.12-7.19 (m, 4H), 6.88 (d, J=8.70 Hz, 4H), 6.53 (s, 1H), 4.62 (s, 4H), 3.71 (s, 6H), 3.65 (q, J=11.01 Hz, 2H) 3.35 (s, 3H).

### Intermediate AW5: 3-(methoxymethoxy)-z-methyl-8-((triisonropylsilyl) ethynyl)naphthalen-1-yl trifluoromethanesulfonate (Int-AW5)

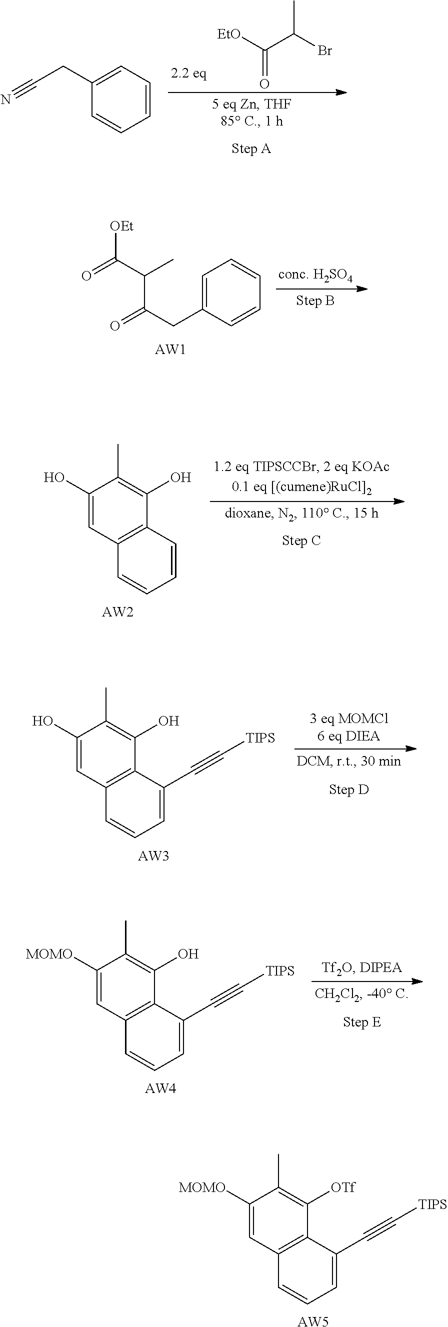

### Step A: Ethyl 2-methyl-3-oxo-4-phenylbutanoate (Int-AW1)

Zinc dust (15 g) was activated by sequential addition, stirred for 5 min and removal, by syringe, of each of 3 M HCl (80 mL), deionized water (80 mL), ethanol (80 mL) and ether (2×80 mL), followed by drying in vacuo to give activated zinc dust (12.84 g). The activated zinc dust (6.42 g, 98 mmol) was suspended in dry THF (100 mL) and heated at 85° C. with stirring, 20 drops of ethyl 2-bromopropanoate was added under N₂. The mixture was stirred at 85° C. for 30 min. 2-phenylacetonitrile (2.3 g, 19.63 mmol) was added in one portion, then ethyl 2-bromopropanoate (7.82 g, 43.2 mmol) in THF (10 mL) was added dropwise over 30 min. The reaction solution was stirred at 85° C. for further 10 min. The mixture was cooled, filtered, diluted with EtOAc (150 mL), and then quenched with aq. K₂CO₃ (100 mL, 50%). Rapidly stirred for 10 mins caused the mixture separated into two layers, the organic layer was decanted and the aqueous layers were further extracted with EtOAc (2×100 mL), dried over MgSO₄, filtered, evaporated under reduced pressure to give the crude product. The crude product was purified by reversed HPLC (Biotage: 40 g Agela, C18, 20-35 μm, Eluent of 66% MeCNH₂O (0.5% TFA) gradient, followed by flash silica gel chromatography 40 g column, Eluent of 6% ethyl acetate in petroleum ether gradient to give ethyl 2-methyl-3-oxo-4-phenylbutanoate. MS (ESI): m/z (M+H)⁺ 221. ¹H NMR (400 MHz, CDCl₃) δ 7.20-7.28 (m, 3H), 7.11-7.17 (m, 2H), 4.09 (q, J=7.1 Hz, 2H), 3.73-3.82 (m, 2H), 3.53-3.58 (m, 1H), 1.24 (d, J=7.2 Hz, 3H), 1.16-1.20 (m, 3H).

### Step B: 2-methylnaphthalene-1,3-diol (Int-AW2)

To a solution of ethyl 2-methyl-3-oxo-4-phenylbutanoate (Int-AW1) (980 mg, 4.45 mmol) was added con. H₂SO₄ (2952 μl, 55.4 mmol) at 0° C. The mixture was stirred at 25° C. for 12 h. The mixture was quenched with ice water (6 mL) and extracted with EtOAc (15 mL×2), dried over Na₂SO₄, filtered and the solvent was evaporated under reduced pressure to give the crude product. The crude product was purified by flash silica gel chromatography 12 g column, Eluent of 10% ethyl acetate in petroleum ether gradient to give 2-methylnaphthalene-1,3-diol. MS (ESI): m/z (M+H)⁺ 175. ¹H NMR (400 MHz, CDC-b) δ 7.99 (d, J=8.4 Hz, 1H), 7.60 (d, J=8.1 Hz, 1H), 7.38 (td, J=7.6, 1.2 Hz, 1H), 7.31 (ddd, J=8.2, 6.8, 1.3 Hz, 1H), 6.78 (s, 1H), 2.32 (s, 3H).

### Step C: 2-methyl-8-((triisoorooylsilyl)ethynyl)naphthalene-1,3-diol (Int-AW3)

To a solution of 2-methylnaphthalene-1,3-diol (Int-AW2) (550 mg, 3.16 mmol) in dioxane (10 mL) was added (bromoethynyl)triisopropylsilane TIPSCCBr) ((990 mg, 3.79 mmol), potassium acetate (620 mg, 6.31 mmol) and dichloro(p-cymene)ruthenium(II) dimer (193 mg, 0.316 mmol) at 25° C. under N₂. The mixture was stirred at 110° C. for 15 h. The mixture was cooled, diluted with EtOAc (20 mL), filtered and the solvent was evaporated under reduced pressure to give the crude product. The crude product was purified by flash silica gel chromatography 12 g column, Eluent of 10% ethyl acetate in petroleum ether gradient to give 2-methyl-8-((triisopropylsilyl)ethynyl)naphthalene-1,3-diol. MS (ESI): m/z (M+H)⁺ 355. ¹H NMR (400 MHz, CDCl₃) δ 9.37 (s, 1H), 7.58 (dd. J=8.2, 1.1 Hz, 1H), 7.45 (dd, J=7.0, 1.2 Hz, 1H), 7.21-7.25 (m, 1H), 6.75 (s, 1H), 4.97 (s, 1H). 2.31 (s, 3H), 1.16-1.20 (m, 21H).

### Step D: 3-(methoxymethoxy)-2-methyl-8-(triisopropylsilyl)ethyny)inaphthalen-1-ol (Int-AW4)

To a solution of 2-methyl-8-((triisopropylsilyl)ethynyl)naphthalene-1,3-diol (Int-AW3) (610 mg, 1.720 mmol) in DCM (8.0 mL) was added DIEA (1.803 mL, 10.32 mmol) and MOM-Cl (600 mg, 7.45 mmol) at 0° C., and the mixture was stirred at 0° C. for 30 min. The reaction mixture was evaporated under reduced pressure to give crude product, which was purified by flash silica gel chromatography 4 g column, Eluent of 0-5% EtOAc/Pet, ether gradient to give 3-(methoxymethoxy)-2-methyl-8-((triisopropylsilyl)ethynyl)naphthalen-1-ol. MS (ESI) m/z: (M+H)⁺ 399. ¹H NMR (400 MHz, CDCl₃) δ 9.24 (s, 1H), 7.59 (d, 8.1 Hz, 1H), 7.41 (d. 6.7 Hz, 1H), 7.19-7.14 (m, 1H), 6.90 (s, 1H), 5.47-5.05 (m, 2H), 3.44 (s, 3H), 2.36-2.19 (m, 3H), 1.15-1.08 (m, 21H).

### Step E: 3-(methoxymethoxy)-2-methyl-8-((triisopropylsilyl)ethynyl)naphthalen--yl trifluoromethanesulfonate (Int-AW5)

To a solution of 3-(methoxymethoxy)-2-methyl-8-((triisopropylsilyl)ethynyl)naphthalen-1-ol (Int-AW4) (310 mg, 0.778 mmol) in DCM (5 mL) was added DIPEA (0.407 mL, 2.333 mmol) and Tf₂O (0.197 mL, 1.167 mmol) at −40° C., and the mixture was stirred at −40° C. for 30 min. The reaction mixture was concentrated in vacuo, and the residue was purified by pre-TLC (Pet, ether:EtOAc=20:1) to give 3-(methoxymethoxy)-2-methyl-8-((triisopropylsilyl)ethynyl)naphthalen-1-yl-trifluoromethanesulfonate. ¹H NMR (ESI): MHz, chloroform-</)=7.71 (t, J=7.6 Hz, 2H), 7.45-7.38 (m, 2H), 7.29 (d, J=2.0 Hz, 1H), 5.27 (s, 2H), 3.54-3.46 (m, 3H), 1.18-1.10 (m, 21H).

### Intermediate AX4: 3-(bis(4-methoxybenzyl)amino)-7-fluoro-8-((triisoaroaylsilyl) ethynyl)isoquinolin-1-yl trifluoromethanesulfonate (Int -X4)

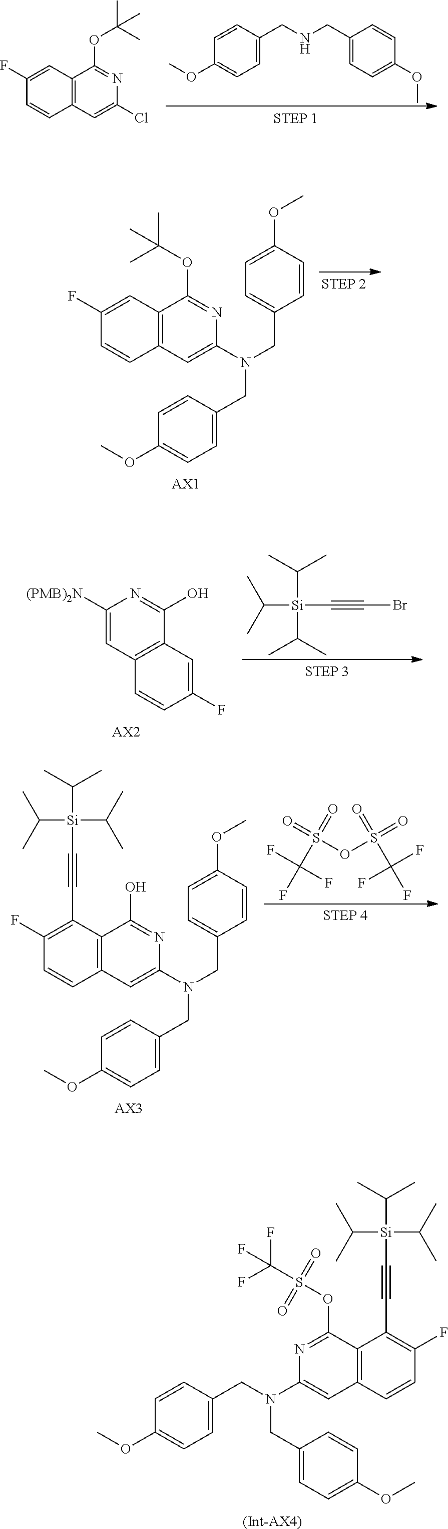

### STEP 1: 1-(tert-butoxy)-7-fluoro-N,N-bis(4-methoxybenzyl)isoquinolin-3-amine Inot-AX1)

A mixture of 1-(tert-butoxy)-3-chloro-7-fluoroisoquinoline (Int-252-1) (4.7 g, 18.53 mmol), bis(4-methoxybenzyl)amnine (7.15 g, 27.8 mmol), sodium t-butoxide (3.56 g, 37.1 mmol) and SPhos Pd G3 (1.446 g, 1.853 mmol) in THF (80 mL) was degassed and backfilled with N₂ for 3 times. The reaction mixture was stirred at 80° C. for 12 h under N₂. The reaction mixture was diluted with EtOAc (100 mL) and filtered. The filtrate was concentrated under reduced pressure to give the residue which was purified by flash silica gel chromatography 40 g column, Eluent of 0-5% EtOAc/Pet, ether gradient (dry loaded) to give 1-(tert-butoxy)-7-fluoro-N,N-bis(4-methoxybenzyl)isoquinolin-3-amine. MS (EST): m/z (M+H)⁺ 475.

### STEP 2: 3-(bis(4-methoxybenzyl)amino)-7-fluoroisoquinolin-1-ol (Int-AX2)

To a solution of 1-(tert-butoxy)-7-fluoro-N,N-bis(4-methoxybenzyl)isoquinolin-3-amine (Int-AX1) (2.0 g, 4.21 mmol) in i-PrOH (10 mL) was added HCl-Dioxane (10 mL). The reaction mixture was stirred at 25° C. for 4 h. The reaction mixture was concentrated under reduced pressure to give the residue which was diluted with EtOAc (20 mL) and NaHC03 (15 mL). The aqueous layer was separated and was extracted with EtOAc (20 mL×3). The combined organic layers were dried over Na₂SO₄ and filtered. The filtrate was concentrated under reduced pressure to give the residue which was purified by flash silica gel chromatography 20 g column, Eluent of 0-36% EtOAc/Pet, ether gradient (dry loaded) to give 3-(bis(4-methoxybenzyl)amino)-7-fluoroisoquinolin-1-ol. MS (ESI): m/(M+H)⁺ 419.

### STEP 3: 3-(bis(4-methoxybenzyl)amino)-7-fluoro-8-((triisopropylsilyl)ethynyl)isoquinolin-1-ol (Int-AX3)

A mixture of 3-(bis(4-methoxybenzyl)amino)-7-fluoroisoquinolin-1-ol (Int-AX2) (1.0 g, 2.390 mmol), (bromoethynyl)triisopropylsilane (0.999 g, 3.82 mmol), potassium acetate (0.469 g, 4.78 mmol) and dichloro(p-cymene)ruthenium(II)dimer (0.146 g, 0.239 mmol) in Dioxane (15 mL) was degassed and backfilled with N2 for 3 times. The reaction mixture was stirred at 100° C. for 15 h under N2 protection. The reaction was filtered. The filtrate was concentrated under reduced pressure to give the residue which was purified by flash silica gel chromatography 20 column, Eluent of 0-23% EtOAc/Pet, ether gradient (dry loaded) to give 3-(bis(4-methoxybenzyl) amino)-7-fluoro-8-((triisopropylsilyl)ethynyl)isoquinolin-1-ol. MS (ESI): m/z (M+H)⁺ 599.

### STEP 4: 3-(bis(4-methoxybenzyl)amino)-7-fluoro-8-((triisopropylsilyl) ethynyl)isoquinolin-1-yl trifluoromethanesulfonate (Int-AX4)

To a solution of 3-(bis(4-methoxybenzyl)amino)-7-fluoro-8-((triisopropylsilyl)ethynyl)isoquinolin-1-ol (Int-AX3) (820 mg, 1.369 mmol) in DCM (15 mL) were added TEA (1.145 mL, 8.22 mmol) and Tf₂O (0.694 mL, 4.11 mmol) at −40° C. under N2 protection. The reaction mixture was stirred at -40° C. for 20 min. The reaction was concentrated under reduced pressure to give the residue which was purified by flash silica gel chromatography 20 g column, Eluent of 0-32% EtOAc/Pet, ether gradient dry loaded to give 3-(bis(4-methoxybenzyl)amino)-7-fluoro-8-((triisopropylsilyl) ethynyl)isoquinolin-1-yl trifluoromethanesulfonate. MS (ESI), m/z (M+H)⁺ 731.

### Intermediate AY5: Synthesis of 2-fluoro-3-(methoxymethoxy)-8-((triisoorooylsilyl)ethyny)inaphthalen-1-yl trifluoromethanesulfonate (Int-AY5)

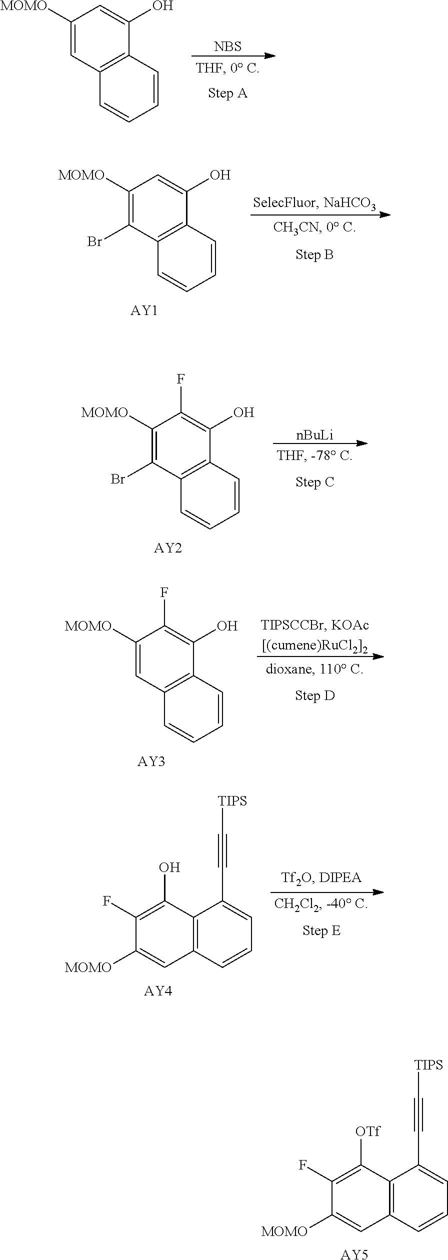

### Step A: 4-bromo-3-(methoxymethoxy)naphthalen-1-ol (Int-AY1)

3-(Methoxymethoxy)naphthalen-1-ol (1.90 g, 9.30 mmol) was dissolved in THF (31 mL) and cooled down to 0° C. N-Bromosuccinimide (1.66 g, 9.30 mmol) was added into the reaction vessel. The resulting mixture was stirred for 2h at 0° C. The diluted product mixture was washed three times with saturated sodium bicarbonate aqueous solution (3.×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 20% ethyl acetate-hexanes, linear gradient to afford 4-bromo-3-(methoxymethoxy)naphthalen-1-ol. ¹H NMR (500 MHz, Chloroform-d) δ 8.23 (d, J=8.6 Hz, 1H), 8.14 (d, J=8.4 Hz, 1H), 7.74-7.53 (m, 1H), 7.53-7.33 (m, 1H), 6.93 (s, 1H), 5.34 (s, 2H), 3.60 (s, 3H).

### Step B: 4-bromo-2-fluoro-3-(methoxymethoxy)naphthalen-1-ol (Int-AY2)

4-Bromo-3-(methoxymethoxy)naphthalen-1-ol (Int-AY1) (1.80 g, 6.36 mmol) was dissolved in acetonitrile (32 mL). Sodium bicarbonate (2.14 g, 25.4 mmol) was added into the reaction vessel and the resulting mixture was cooled down to 0° C. 1-Chloromethyl-4-fluoro-1,4-diazoniabicyclo[2.2.2]octane bis(tetrafluoroborate) (3.38 g, 9.54 mmol) was added in 3 equal portions. The resulting mixture was stirred for 1 h at 0° C. The product mixture was diluted with ether (100 mL) and filtered. The filtrate was concentrated to dryness and redissolved in ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford the desired product. 4-bromo-2-fluoro-3-(methoxymethoxy)naphthalen-1-ol. ¹H NMR (500 MHz, Chloroform-d) δ 8.09 (d, J=7.8 Hz, 1H), 7.97 (d, J=7.9 Hz, 1H), 7.74 (t, J=7.6 Hz, 1H), 7.60 (t, J=7.6 Hz, 1H), 6.00 (t, J=2.0 Hz, 1H), 5.41 (d, J=6.5 Hz, 1H), 5.36 (d, J=6.4 Hz, 1H), 3.63 (d, J=1.4 Hz, 3H).

### Step C: 2-fluoro-3-(methoxymethoxy)naphthalen-1-ol (Int-AY3)

4-Bromo-2-fluoro-3-(methoxymethoxy)naphthalen-1-ol (Int-AY2) (1,38 g, 4.57 mmol) was added into a 250 mL round-bottomed flask. The reaction vessel was evacuated and backfilled with a balloon of nitrogen three times. THF (23 mL) was added to dissolve the starting material and the resulting mixture was cooled down to −78° C. n-Butyl)ithium (2.5 M in hexanes, 3.66 mL, 9.14 mmol) was added dropwise into the reaction vessel. The resulting mixture was stirred for 30 min then quenched with water (10 mL). The quenched product mixture was extracted three times with DCM (3-30 mL). The organic layer was dried over sodium sulfate. The dried solution was filtered and the filtrate was concentrated to dryness. The residue obtained was purified by flash column chromatography with 40-g silica gel column, eluting with hexanes initially, grading to 50% ethyl acetate-hexanes, linear gradient to afford the desired product. 2-fluoro-3-(methoxymethoxy)naphthalen-1-ol. ¹H NMR (500 MHz, Chloroform-d) δ 8.09 (d, J=8.5 Hz, 1H), 8.02 (d, J=8.4 Hz, 1H), 7.56 (t, J=7.6 Hz, 1H), 7.45 (t. J=7.7 Hz, 1H), 6.84 (d, J=6.4 Hz, 1H), 5.29 (s, 2H), 5.24 (s, 1H), 3.60 (d, J=1.2 Hz, 3H).

### Step D: 2-fluoro-3-(methoxymethoxy08-(triisoproDvisilyl)ethynyl)naphthalen-1-ol (Int-AY4)

To 2-fluoro-3-(methoxymethoxy)naphthalen-1-ol (Int-AY3) (529 mg, 2.38 mmol) and dichloro(p-cymene)ruthenium(II) dimer (146 mg. 0.238 mmol) and potassium acetate (467 mg. 4.76 mmol) in dioxane (4 mL), (bromoethynyl)triisopropylsilane (746 mg, 2.86 mmol) was added. The flask was thoroughly flushed with nitrogen and reaction stirred for 16h at 110° C. The reaction mixture was filtered through a bed of CELITE. The filter bed was washed with excess DCM. The filtrate was concentrated and directly purified on a silica gel column using 0-10% Hex- EtOAc to afford 2-fluoro-3-(methoxymethoxy)-8-((triisopropylsilyl)ethynyl)naphthalen-1-ol. ¹H NMR (500 MHz. Chloroform-d) δ 9.10 (s, 1H), 8.03 (d, J=8.5 Hz, 1H), 7.58 (d, J=7.1 Hz, 1H), 7.42 (dd, J=8.5, 7.1 Hz, 1H), 6.98 (d, J=7.2 Hz, 1H), 5.30 (s, 2H), 3.57 (s, 3H), 1.36-1.13 (m, 21H).

### Step E: 2-fluoro-3-(methoxymethoxyl˜8-(triisoproovisilyl)ethynyl)naphthalen-1-yl trifluoromethanesulfonate (Int-AY5)

To 2-fluoro-3-(methoxymethoxy)-8-((triisopropylsilyl)ethynyl)naphthalen-1-ol (Int-AY4) (172 mg, 0.427 mmol) and DIPEA (224 μL. 1.28 mmol) in DCM (2.1 mL), triflic anhydride (trifluoromethanesulfonic anhydride) (108 μL, 0.641 mmol) was added at −40° C. and stirred for an hour. The reaction mixture was filtered through abed of CELITE. The filter bed was washed with excess DCM. The filtrate was concentrated and directly purified on a silica gel column using 0-10% Hex-EtOAc to afford 2-fluoro-3-(methoxymethoxy)-8-((triisopropylsilyl)ethynyl)naphthalen-1-yl trifluoromethanesulfonate. ¹H NMR (499 MHz, Chloroform-d) δ 8.08 (d, J=8.6 Hz, 1H), 7.84 (d, J=7.2 Hz, 1H), 7.58 (d, J=6.9 Hz. 1H), 7.55 (t, J=7.8 Hz, 1H), 5.32 (d, J=1.2 Hz, 3H), 3.59 (d, J=1.2 Hz, 2H), 1.38-1.02 (m, 21H).

### Intermediate AZ3: N-(l-bromo-8-chloroisoquinolin-3-yl)acetamide (Int-AZ3)

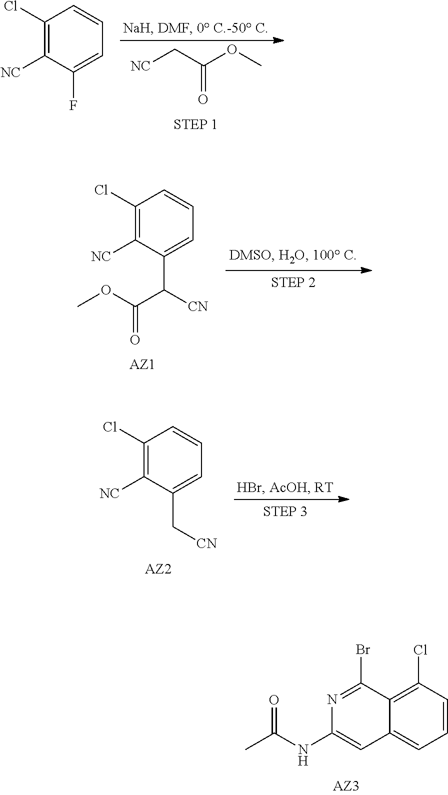

### STEP 1: Methyl 2-(3-chloro-2-cyanophenyl)-2-cyanoacetate (Int-AZ)

To a flask was added sodium hydride (2.057 g, 51.4 mmol) and it was cooled to 0° C. A mixture of methyl 2-cyanoacetate (5.10 g, 51.4 mmol)) in DMF (15 ml)) was added to the flask dropwise. The mixture was allowed to warm to room temperature for one hour. After 1 hour, 2-chloro-6-fluorobenzonitrile (4 g, 25.7 mmol) in DMF (15 ml)) was added. The mixture was then heated to 50° C. for 4 hours. The reaction mixture was cooled to room temperature, the mixture was carefully quenched with 2N HCl and then ethyl acetate was added. The organic layer was then separated, dried over magnesium sulfate, filtered and concentrated under reduced pressure to yield methyl 2-(3-chloro-2-cyanophenyl)-2-cyanoacetate. MS (ESI): m/z (M+H+Na)⁺ 257.

### STEP 2: 2-chloro-6-(cyanomethyl)benzonitrile (Int-AZ2)

To a vial containing methyl 2-(3-chloro-2-cyanophenyl)-2-cyanoacetate (Int-AZ1) (550 mg, 2.344 mmol) was added deuterated DMSO (3.8 ml) and then Water (0.5 ml). The mixture was heated to 100° C. for 18 hrs. The mixture was allowed to cool to room temperature and quenched with water. The resulting solid was filtered and then washed with water. The solids were then dried under vacuum to yield the desired product 2-chloro-6-(cyanomethyl)benzonitrile. MS (ESI): m/z (M+H+Na)⁺ 199.

### STEP 3: N-(1-bromo-8-chloroisoquinolin-3-yl)acetamide (Int-AZ3)

HBr in 33% acetic acid (3 ml) was added to 2-chloro-6-(cyanomethyl)benzonitrile (Int-AZ2) (400 mg, 2.265 mmol) and stirred at rt for overnight. The reaction mixture was slowly quenched with sat NaHCO₃ solution with stirring and the solid precipitated was filtered and dried under vacuum. The resulting solid material was washed with diethyl ether and dried under vacuum to yield N-(1-bromo-8-chloroisoquinolin-3-yl)acetamide. MS (EST): m/z (M+H)⁺ 299.

### Intermediate BA13: 7-fluoro-6-methyl-1-(tetrahydro-2H-pyran-2-yl)-5-(trifluoromethyl)-1H-indazol-4-yl trifluoromethanesulfonate (Int BA13)

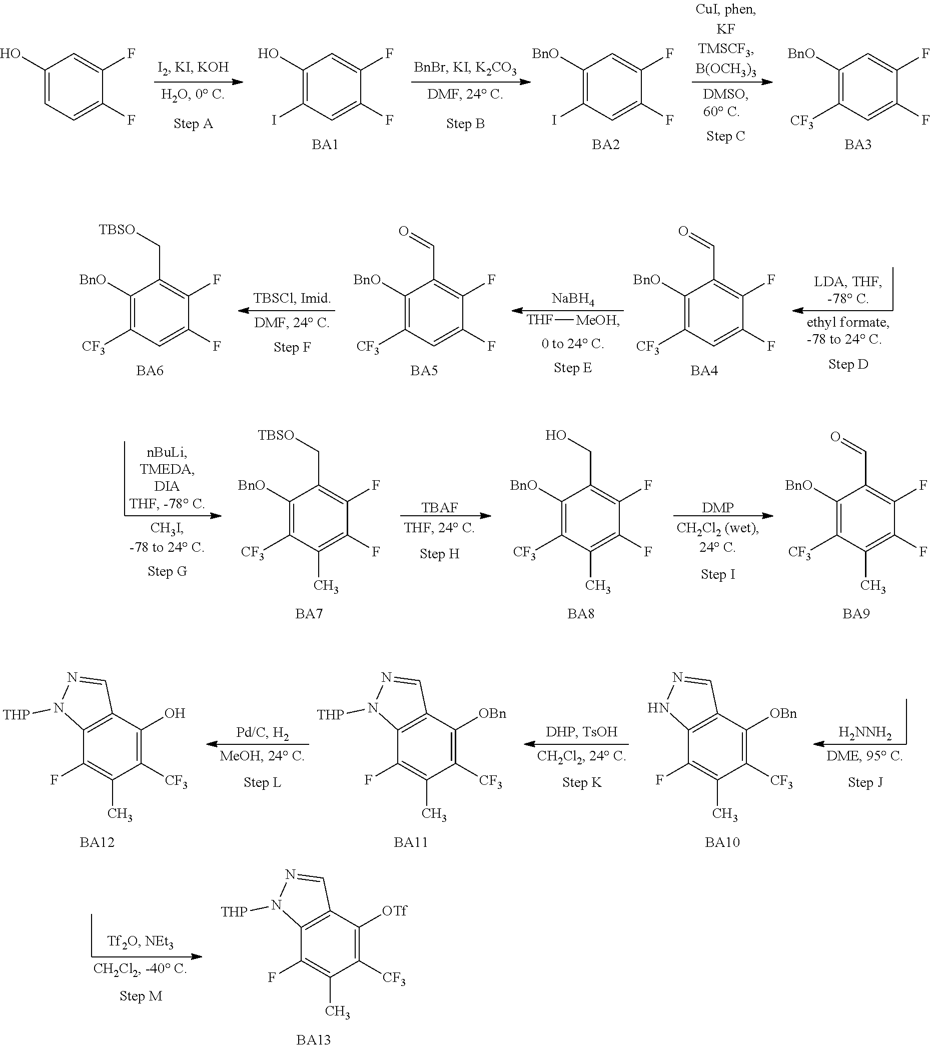

### Step A: 4,5-difluoro-2-iodophenol (Int BA1)

3.4-Difluorophenol (5.00 g, 38.4 mmol) was dissolved in water (192 mL) in a 1-L flask. The resulting mixture was cooled down to 0° C. Potassium iodide (6.38 g, 38.4 mmol) and diiodine (9.75 g, 38.4 mmol) were added into the reaction vessel and the resulting mixture was stirred until all material fully dissolved. In an Erlenmeyer flask, potassium hydroxide (4.31 g, 77.0 mmol) was dissolved in water (192 mL). After the KOH solution cooled down to room temperature, the solution was transferred to an addition funnel. The KOH solution was added dropwise over 45 min into the reaction mixture at 0° C. The resulting mixture was stirred for 3h while warming up to room temperature. The product mixture was acidified to pH<3 with saturated ammonium chloride aqueous solution and 0.1 N hydrochloric acid aqueous solution. The acidified mixture was extracted three times with chloroform (3′100 mL). The organic laver was dried over sodium sulfate. The dried solution was filtered and the filtrate was concentrated to dryness. The residue obtained was purified by flash column chromatography with 220-g silica gel column, eluting with hexanes initially, grading to 20% ethyl acetate-hexanes, linear gradient to afford 4,5-difluoro-2-iodophenol. ¹H NMR (500 MHz, DMSO-d₆) δ 10.69 (s, 1H), 7.77 (t, J - 9.6 Hz, 1H), 6.85 (dd, J=12.3, 7.2 Hz, 1H).

### Step B: 1-(benzyloxy)-4.5-difluoro-2-iodobenzene (Int-BA2)

4,5-Difluoro-2-iodophenol (Int-BA1) (8.77 g, 34.3 mmol) was added in a 500 mL round-bottomed flask. DMF (101 mL) was added into the reaction vessel. The resulting mixture was stirred until all the starting material was dissolved. Potassium carbonate (14.2 g, 103 mmol) and potassium iodide (0.569 g, 3.43 mmol) was added into the reaction mixture. Upon vigorous stirring, (bromomethyl)benzene (BnBr) (4.07 mL, 34.3 mmol) was added dropwise into the reaction vessel. The resulting mixture was stirred for 16h at 24° C. The product mixture was diluted with ether (400 mL) and washed 5 times with water (5×*200 mL). The organic layer was cut with pentane (200 mL) and dried over sodium sulfate. The dried solution was filtered and the filtrate was concentrated to dryness. The residue obtained was purified by flash column chromatography with 220-g silica gel column, eluting with hexanes initially, grading to 20% ether-hexanes, linear gradient to afford 1-(benzyloxy)-4,5-difluoro-2-iodobenzene. ¹H NMR (500 MHz, Acetonitrile-d₃) δ 7.77 (t, J=9.4 Hz, 1H), 7.53 (d, J=7.5 Hz, 2H), 7.45 (t, J=7.4 Hz, 2H), 7.39 (t, J=7.4 Hz, 1H), 7.03 (dd, J=12.6, 7.0 Hz, 1H), 5.15 (s, 2H).

### Step C: 1-(benzyloxy)-4,5-difluoro-2-(trifluoromethyl)benzene (Int-BA3)

1-(Benzyloxy)-4,5-difluoro-2-iodobenzene (Int-BA2) (9.0) g, 26.0 mmol), copper(I) iodide (1.981 g, 10.4 mmol), 1.10-phenanthroline (phen) (1.874 g, 10.4 mmol), and potassium fluoride (7.55 g, 130 mmol) were added into a microwave tube. The reaction vessel was evacuated and backfilled with a balloon of nitrogen three times. DMSO (65.0 mL), trimethyl borate (14.5 mL, 130 mmol), and trimethyl(trifluoromethyl)silane (TMSCF₃) (19.2 mL, 130 mmol) were added into the reaction vessel. The resulting mixture was heated at 60° C. for 16h. The product mixtures were combined and diluted with ether (500 mL). The diluted product mixture was washed three times with saturated 10% ammonia aqueous solution (3×200 mL). The organic layer was dried over sodium sulfate. The dried solution was filtered and the filtrate was concentrated to dryness. The residue obtained was purified by flash column chromatography with 220-g silica gel column, eluting with hexanes initially, grading to 20% ether-hexanes, linear gradient to afford 1-(benzyloxy)-4,5-difluoro-2-(trifluoromethyl)benzene. ¹H NMR (500 MHz, Acetonitrile-di) δ 7.71-7.56 (m, 1H), 7.50-7.38 (m, 5H), 7.22 (dd, J=12.3, 6.6 Hz, 1H), 5.20 (s, 2H).

### Step D: 2-(benzyloxy)-5,6-difluoro-3-(trifluoromethyl)benzaldehyde (Int-BA4)

1-(Benzyloxy)_{4,5}-difluoro-2-(trifluoromethyl)benzene (Int-BA3) (6.39 g, 22.2 mmol) was added into a 500 mL round-bottomed flask. The reaction vessel was evacuated and backfilled with a balloon of nitrogen three times. Tetrahydrofuran (55.4 mL) was added into the reaction vessel. The resulting solution was cooled down to −78° C. Lithium diisopropylamide (2M solution in THF, 14.4 mL, 28.8 mmol) was added dropwise into the reaction vessel. The resulting mixture was stirred for 30 min. Ethyl formate (17.9 mL, 222 mmol) was added into the reaction vessel. The resulting mixture was warmed up to 24° C. The product mixture was quenched with saturated ammonium chloride aqueous solution (1 mL). The quenched product mixture was extracted three times with DCM (3×30 mL). The organic layer was dried over sodium sulfate. The dried solution was filtered and the filtrate was concentrated to dryness. The residue obtained was purified by flash column chromatography with 24-g silica gel column, eluting with hexanes initially, grading to 20% ethyl acetate-hexanes, linear gradient to afford 2-(benzyloxy)-5,6-difluoro-3-(trifluoromethyl)benzaldehyde. ¹H NMR (500 MHz, Acetonitrile-di) 8.10.24 (s, 1H), 7.95 (t.. =9.4 Hz, 1H), 7.63-7.44 (m, 5H), 5.10 (s, 2H).

### Step E: (2-(benzyloxy)-5,6-difluoro-3-(trifluoromethyl)phenyl)methanol (Int-BA5)

2-(Benzyloxy)-5,6-difluoro-3-(trifluoromethyl)benzaldehyde (Int-BA4) (4.01 g, 12.7 mmol) was dissolved in THF (47.6 mL) and Methanol (15.9 mL). The resulting solution was cooled down to 0° C. Sodium tetrahydroborate (1.44 g, 38.0 mmol) was added into the reaction vessel. The resulting mixture was allowed to warm up to room temp. The product mixture was quenched with saturated ammonium chloride aqueous solution (1 mL). The quenched product mixture was extracted three times with DCM (3-30 mL). The organic layer was dried over sodium sulfate. The dried solution was filtered and the filtrate was concentrated to dryness. The residue obtained was purified by flash column chromatography with 40-g silica gel column, eluting with hexanes initially, grading to 50% ethyl acetate-hexanes, linear gradient to afford (2-(benzyloxy)-5.6-difluoro-3-(trifluoromethyl)phenyl)methanol. ¹H NMR (500 MHz, Chloroform-d) δ 7.68-7.37 (m, 6H), 5.11 (s, 2H), 4.81 (d, J=1.9 Hz, 2H).

### Step F: ((2-(benzyloxy)-5.6-difluoro-3-(trifluoromethyl)benzyl)oxy)(ert-butyl)dimethylsilane (Int-BA6)

(2-(Benzyloxy)-5,6-difluoro-3-(trifluoromethyl)phenyl)methanol (Int-BA5) (4.07 g, 12.8 mmol) was dissolved in DMF (25.6 mL). Imidazole (2.61 g, 38.4 mmol) and tert-butylchlorodimethylsilane (2.89 g, 19.2 mmol) were added into the reaction vessel. The resulting mixture was stirred for 1 h. The product mixture was diluted with ethyl acetate (100 mL). The diluted product mixture was washed three times with saturated ammonium chloride aqueous solution (3×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 20% ethyl acetate-hexanes, linear gradient to afford ((2-(benzyloxy)-5,6-difluoro-3-(trifluoromethyl)benzyl)oxy)tert-butyl)dimethylsilane. ¹H NMR (500 MHz, Acetonitrile-d3) δ 7.71-7.63 (m, 1H), 7.58-7.38 (m, 5H), 5.14 (s, 2H), 4.85 (d, J=2.0 Hz, 2H), 0.90 (s, 9H), 0.14 (s, 6H).

### Step G: ((2-(benzyloxy)-5.6-difluoro-4-methyl-3-(trifluoromethyl)benzyl)ox)(tert-butyl)dimethylsilane (Int-BA7)

((2-(Benzyloxy)-5,6-difluoro-3-(trifluoromethyl)benzyl)oxy)(tert-butyl)dimethylsilane (Int-BA6) (1.00 g, 2.31 mmol) was added into a 30 mL vial. The reaction vessel was evacuated and backfilled with a balloon of nitrogen three times. THF (12 mL) was added into the reaction vessel. The resulting solution was cooled down to −78° C. N¹, N¹, N². N²-tetramethylethane-1,2-diamine (0.693 mL, 4.62 mmol) and diisopropylamine (DIA) (0.032 mL, 0.23 mmol) were added into the reaction vessel. The resulting mixture was stirred for 10 min. Then n-butylthium (1.11 mL, 2.77 mmol) was added dropwise into the reaction vessel. The resulting mixture was stirred for 2h at −78° C. Then iodomethane (0.432 mL, 6.94 mmol) was added into the reaction vessel and the resulting mixture was allowed to warm up to room temperature. The product mixture was quenched with saturated ammonium chloride aqueous solution (1 mL). The quenched product mixture was extracted three times with DCM (3×30 mL). The organic layer was dried over sodium sulfate. The dried solution was filtered and the filtrate was concentrated to dryness. The residue obtained was purified by flash column chromatography with 24-g silica gel column, eluting with hexanes initially, grading to 20% ethyl acetate-hexanes, linear gradient to afford ((2-(benzyloxy)-5,6-difluoro-4-methyl-3-(trifluoromethyl)benzyl)oxy)tert-butyl)dimethylsilane. ¹H NMR (500 MHz, Chloroform-d) δ 7.58-7.35 (m, 5H), 5.08 (s, 2H), 4.77 (s, 2H), 2.45 (t, J=2.9 Hz, 3H), 0.91 (s, 9H), 0.14 (s, 6H).

### Step H; (2-(benzyloxy)-5.6-difluoro-4-methyl-3-(trifluoromethyl)phenyl)methanol (Int-BA5)

((2-(Benzyloxy)-5.6-difluoro-4-methyl-3-(trifluoromethyl)benzyl)oxy)tert-butyl)dimethylsilane (Int-BA7) (1.00 g, 2.24 mmol) was added into a 30 mL vial. THF (7.5 mL) was added into the reaction vessel. Then tetrabutylammonium fluoride (1 M in THF, 2.24 mL, 2.24 mmol) was added into the reaction vessel. The resulting mixture was stirred for 5 min at rt. The product mixture was diluted with ethyl acetate (100 mL). The diluted product mixture was washed three times with saturated ammonium chloride aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 40% ethyl acetate-hexanes, linear gradient to afford (2-(benzyloxy)-5,6-difluoro-4-methyl-3-(trifluoromethyl)phenyl)methanol. ¹H NMR (500 MHz, Acetonitrile-d3) δ 7.65-7.19 (m, 5H), 5.04 (s, 2H), 4.69 (d, J=5.3 Hz, 2H), 3.43 (t, J=5.6 Hz, 1H), 2.45 (q, J=2.9 Hz. 3H).

### Step 1: 2-(benzyloxy)-5,6-difluoro-4-methyl-3-(trifluoromethyl)benzaldehyde (Int-BA9)

(2-(Benzyloxy)-5,6-difluoro-4-methyl-3-(trifluoromethyl)phenyl)methanol (Int-BA8) was dissolved in dichloromethane (water saturated) (11.5 mL) in a 40 mL vial. Dess-Martin periodinane (1.47 g, 3.46 mmol) was added into the reaction vessel and the resulting mixture was stirred for 2h at 24° C. The product mixture was diluted with diethyl ether (20 mL). The diluted product mixture was washed with a 1:1 saturated sodium bicarbonate aqueous solution and saturated sodium thiosulfate aqueous solution (20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford 2-(benzyloxy)-5,6-difluoro-4-methyl-3-(trifluoromethyl)benzaldehyde. ¹H NMR (500 MHz, Acetonitrile-d₃) δ 10.15 (s. 1H), 7.64-7.25 (m, 5H), 5.05 (s, 2H), 2.51 (p, J=2.9 Hz, 3H).

### Step J: 4-(benzyloxy)-7-fluoro-6-methyl-5-(trifluoromethyl)-1H-indazole (Int-BA 10)

2-(Benzyloxy)-5,6-difluoro-4-methyl-3-(trifluoromethyl)benzaldehyde (Int-BA9) (660 mg, 2.00 mmol) was added into a 25 mL round bottomed flask with two necks equipped with a reflux condensor. The reaction vessel was evacuated and backfilled with a balloon of nitrogen three times. DME (5.0 mL) and hydrazine hydrate (972 μL, 20.0 mmol) were added into the reaction vessel then the resulting mixture was heated at reflux temp (jacket temp 95° C.) for 1 h. The product mixture was cooled down to room temperature. The cooled product mixture was diluted with ether (20 mL) and hexanes (20 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 60% ethyl acetate-hexanes, linear gradient to afford 4-(benzyloxy)-7-fluoro-6-methyl-5-(trifluoromethyl)-1H-indazole. ¹H NMR (499 MHz, Acetonitrile-d3) δ 11.72 (s, 1H), 8.31 (s, 1H), 7.55 (d, J=7.0 Hz, 2H), 7.44 (dt, J=15.2, 7.2 Hz, 3H), 5.42 (s, 2H), 2.47 (q, J=3.0 Hz. 3H).

### Step K: 4-(benzyloxy)-7-fluoro-6-methyl-1-(tetrahydro-2H-pyran-2-yl)-5-(trifluoromethyl)-1H-indazole (Int-BA11)

4-(Benzyloxy)-7-fluoro-6-methyl-5-(trifluoromethyl)-1H-indazole (Int-BA10) (597 mg, 1.84 mmol) was added into a 30 mL vial. The reaction vessel was evacuated and backfilled with a balloon of nitrogen three times. DCM (6.1 mL), 4-methylbenzenesulfonic acid hydrate (35.0 mg, 0.184 mmol), and 3.4-dihydro-2H-pyran (336 μL, 3.68 mmol) were added into the reaction vessel sequentially. The resulting mixture was stirred for 1 h. The product mixture was diluted with ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford 4-(benzyloxy)-7-fluoro-6-methyl-1-(tetrahydro-2H-pyran-2-yl)-5-(trifluoromethyl)-1H-indazole. ¹H NMR (500 MHz, Acetonitrile-d3) δ 8.26 (d, J=1.9 Hz, 1H), 7.54 (d, J=7.4 Hz, 2H), 7.44 (dt, J=13.9, 7.2 Hz, 3H), 5.91 (d, J=9.9 Hz, 1H), 5.36 (s, 2H), 3.99 (d, J=11.7 Hz, 1H), 3.76 (td, J=11.1, 3.2 Hz, 1H), 2.69-2.37 (m, 4H), 1.88-1.43 (M, 3H).

### Step L: 7-fluoro-6-methyl-1-(tetrahydro-2H-pyran-2-yl)-5-(trifluoromethyl)-1H-indazol-4-ol (Int-BA12)

4-(Benzyloxy)-7-fluoro-6-methyl-1-(tetrahydro-2H-pyran-2-yl)-5-(trifluoromethyl)-1H-indazole (Int-BA11) (750 mg, 1.84 mmol) and palladium on activated charcoal (10 wt %, 98 mg, 0.092 mmol) were added into an 8 mL vial. The reaction vessel was evacuated and backfilled with a balloon of nitrogen three times. Methanol (9.2 mL) was added into the reaction vessel. The reaction vessel was evacuated and backfilled with a balloon of hydrogen three times. The resulting mixture was stirred for 6h. LCMS showed full conversion. The product mixture was filtered and the filtrate was concentrated to dryness. The residue obtained was purified by flash column chromatography with 12-g silica gel column, eluting with hexanes initially, grading to 40% ethyl acetate-hexanes, linear gradient to afford the desired product 7-fluoro-6-methyl-1-(tetrahydro-2H-pyran-2-yl)-5-(trifluoromethyl)-1H-indazol-4-ol. ¹H NMR (500 MHz, Chloroform-d) δ 8.18 (s, 1H), 6.84-6.45 (m, 1H), 5.88 (dd, J=10.1, 2.5 Hz, 1H), 4.09 (d, J=11.6 Hz, 1H), 3.77 (t, J=10.7 Hz, 1H), 2.60 (q, J=14.3, 13.0 Hz, 1H), 2.45-2.32 (m, 3H), 2.19 (s, 1H), 2.07 (d, J=1.3 Hz, 5H), 1.77 (q, J=12.4, 11.0 Hz, 2H).

### Step M: 7-fluoro-6-methyl-1-(tetrahydro-2H-pyran-2-yl)-5-(trifluoromethyl)-1H-indazol-4-yl trifluoromethanesulfonate (Int-BA13)

To 7-fluoro-6-methyl-1-(tetrahydro-2H-pyran-2-yl)-5-(trifluoromethyl)-1H-indazol-4-ol (Int-BA12) (470 mg, 1.48 mmol) and DIEA (774 μL, 4.43 mmol) in DCM (7.4 mL), triflic anhydride (374 μL, 2.22 mmol) was added at -40° C. and stirred for an hour. The reaction mixture was filtered through a bed of CELITE. The filter bed was washed with excess DCM. The filtrate was concentrated and directly purified on a silica gel column using 0-10% Hex-EtOAc to afford 7-fluoro-6-methyl-1-(tetrahydro-2H-pyran-2-yl)-5-(trifluoromethyl)-1H-indazol-4-yl trifluoromethanesulfonate. ¹H NMR (499 MHz, Acetonitrile-d3) δ 8.27 (s, 1H), 5.95 (d, J=9.7 Hz, 1H), 4.00 (d, J=11.6 Hz, 1H), 3.78 (td, J=11.1, 3.3 Hz, 1H), 2.73-2.32 (m, 4H), 1.87-1.56 (m, 4H).

### Intermediate BC4: 5-chloro-1-((trifluoromethyl)sulfonyl)-1H-benzo[f]indazol-4-yl hifluoromethanesulfonate (Int-BC4)

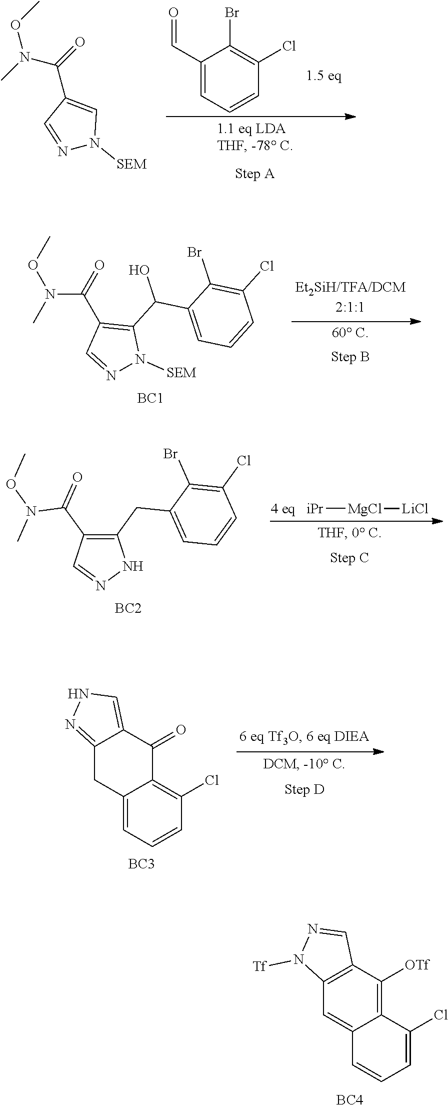

### Step A: 5-((2-bromo-3-chlorophenyl)(hydroxy)methyl-N-methoxy-N-methyl-1-((2-(trimethylsilyl)ethoxy)methyl)-1H-pyrazole-4-carboxamide (Int BC1)

To a stirred solution of N-methoxy-N-methyl-1-((2-(trimethylsilyl)ethoxy)methyl)-1H-pyrazole-4-carboxamide (5 g, 17.52 mmol) in THF (50 mL) was added lithium diisopropylamide (9.20 mL, 18.39 mmol) at -78° C. under N₂, the reaction was stirred at −78° C. for 0.5 h. Then 2-bromo-3-chlorobenzaldehyde (5.77 g, 26.3 mmol) was added at −78° C., the reaction was stirred at −78° C. for 0.5 h. The mixture was quenched with NH₄Cl (20 mL) and extracted with EtOAc (40 mL×3), the organic layers were washed with sat. NaCl (20 mL) dried over Na₂SO₄ and concentrated. The residue was purified by flash silica gel chromatography Silica-CS (80 g), Eluent of 0-36% Ethyl acetate/Petroleum ether gradient to give 5-((2-bromo-3-chlorophenyl)(hydroxy)methyl)-N-methoxy-N-methyl-1-((2-(trimethylsilyl)ethoxy)methyl)-1H-pyrazole-4-carboxamide. MS (ESI): m/z (M+H)⁺ 504, 506. ¹H NMR (500 MHz, CDCl₃) δ 8.10-8.17 (m, 1H), 7.50 (dd, J=7.9, 1.6 Hz, 1H), 7.31-7.37 (m, 1H), 7.25-7.29 (m, 1H), 6.74 (br d, J=10.7 Hz, 1H), 6.59 (d, J=10.7 Hz, 1H), 5.44-5.53 (m, 2H), 3.75-3.80 (m, 3H), 3.52 (ddd, J=10.8, 9.4, 6.0 Hz, 1H), 3.43-3.46 (M. 3H). 3.33-3.42 (m, 1H), 0.80 (ddd. J=13.8, 11.1, 6.0 Hz, 1H), 0.65 (ddd, J=13.8, 10.9, 5.5 Hz, 1H), 0.014.04 (m, 9H).

### Step B: 5-(2-bromo-3-chlorobenzyl)-N-methoxy-N-methyl-1H-pyrazole-4-carboxamide (Int-BC2)

To a solution of 5-((2-bromo-3-chlorophenyl)(hydroxy)methyl)-N-methoxy-N-methyl-1 2-((2(trimethylsilyl)ethoxy)methyl)-1H-pyrazole-4-carboxamide (Int-BC1) (6.3 g, 12.48 mmol) in DCM (40 mL) was added triethylsilane (80 mL, 501 mmol) and TFA (40 mL, 519 mmol). The mixture was stirred at 60° C. for 16 h. TLC showed new spots were formed and no starting material was remained. The reaction mixture was concentrated in vacuo and the residue was diluted with EtOAc (100 mL). The reaction mixture was acidified to pH -8 with aq. NaOH (2 M). The organic layer was washed with brine (20 mL×2), dried with Na₂SO₄, filtered and concentrated in vacuo, the residue was purified by flash silica gel chromatography Column Silica-CS (40 g), Eluent of 0-25% Ethyl acetate/Petroleum ether gradient to give 5-(2-bromo-3-chlorobenzyl)-N-methoxy-N-methyl-1H-pyrazole-4-carboxamide. MS (ESI): m/z (M+H)⁺ 358, 360.

### Step C: 5-chloro-2,9-dihydro-4H-benzo[f]indazol-4-one (Int-BC3)

To a stirred solution of 5-(2-bromo-3-chlorobenzyl)-N-methoxy-N-methyl-1H-pyrazole-4-carboxamide (Int-BC2) (1.2 g, 3.35 mmol) in THF (30 mL) was added isopropylmagnesium(II) lithium chloride (10.30 mL, 13.38 mmol) at 0° C., after the addition was finished, the reaction was stirred at 0° C. for 2 h under N₂. The mixture was diluted with EtOAc (50 mL) and quenched with HCl (aq. 1 M) to pH -5, then basified with sat. NaHCO₃(aq.), the organic layer was washed with brine (20 mL), dried over Na₂SO₄ and concentrated. The residue was washed with EtOAc (10 mL) to give 5-chloro-2,9-dihydro-4H-benzo[f]indazol-4-one which was used directly in the next step without further purification. MS (ESI): m/z (M+H)⁺ 219.

### Step D: 5-chloro-1-((trifluoromethyl)sulfonyl)-1H-benzo[f]indazol-4-yl trifluoromethanesulfonate (Int-BC4)

To a solution of 5-chloro-2,9-dihydro-4H-benzo[f]indazol-4-one (Int-BC3) (430 mg, 1.967 mmol) in DCM (20 mL) was added DIEA (2.061 mL, 11.80 mmol) at −10° C., and the mixture was stirred at −10° C. for 10 min, then Tf₂O (1.994 mL, 11.80 mmol) was added to the above mixture, and the reaction was stirred at −10° C. for 1 h under N2 atmosphere. The reaction was diluted with DCM (30 mL), washed with brine (10 mL), the organic layer was dried over Na₂SO₄ and concentrated in vacuo, the residue was purified by flash silica gel chromatography 12 g column, Eluent of petroleum ether gradient to give 5-chloro-1-((trifluoromethyl)sulfonyl)-1H-benzo[f]indazol-4-yl trifluoromethanesulfonate. ¹H NMR (500 MHz, CDCl₃) &: 8.72 (s, 1H), 8.55 (s, 1H), 8.04 (d, J=8.5 Hz, 1H), 7.79 (dd, J=7.4, 1.0 Hz, 1H), 7.62 (dd, J=8.3, 7.6 Hz, 1H).

### Intermediate BD4: 1-bromo-38-dichloronaphthalene (Int-BD4)

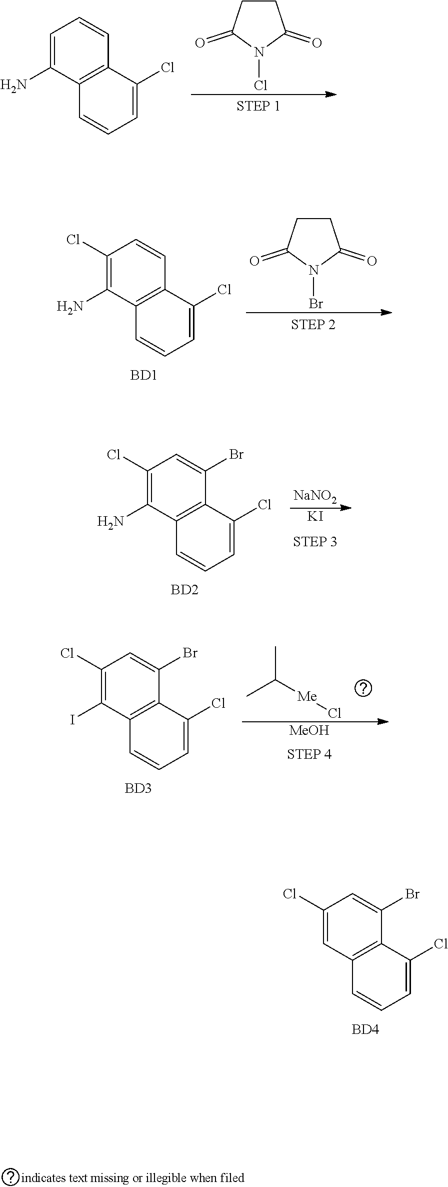

### STEP 1: 2.5-dichloronaphthalen-1-amine (Int-BD1)

To a solution of 5-chloronaphthalen-1-amine (700 mg, 3.94 mmol) in THF (10 ml) was added 1-chloropyrrolidine-2,5-dione (526 mg, 3.94 mmol) in THF (5 ml) dropwise at 20° C. N2 atmosphere. The mixture was stirred at 20° C. for 3 h. The reaction was cooled to rt, the mixture was concentrated, the residue was purified by flash silica gel chromatography column Silica-CS(10 g), Eluent of 4% Ethyl acetate/Petroleum ether gradient, to give 2,5-dichloronaphthalen-1-anine. ¹H NMR (500 MHz, METHANOL-d₆) δ ppm 7.39 (dd, J=−8.54, 7.48 Hz, 1H) 7.43 (d, J=9.16 Hz, 1H) 7.52-7.58 (m, 2H) 8.01 (d, J=8.54 Hz, 1H).

### STEP 2: 4-bromo-2,5-dichloronaphthalen-1-amine (Int-BD2)

A solution of 2,5-dichloronaphthalen-1-amine (Int-BD1) (390 mg, 1.839 mmol) and N-bromosuccinimide (327 m. 1.839 mmol) in DMF (8 ml) was stirred at 50° C. for 1 h. The reaction was cooled to rt, the mixture was concentrated, the residue was purified by pre-TLC (Pet, ether/EtOAc=5:1) to give 4-bromo-2,5-dichloronaphthalen-1-amine. ¹H NMR (500 MHz, CHLOROFORM-d) δ ppm 4.59 (br s, 2H) 7.38 (dd, J=8.39, 7.63 Hz, 1H) 7.62-7.66 (m, 1H) 7.77 (dd, J=8.54, 1.22 Hz, 1H) 7.82 (s, 1H).

### STEP 3: 4-bromo-2,5-dichloro-1-iodonaphthalene (Int-BD3)

To a mixture of 4-bromo-2,5-dichloronaphthalen-1-amine (Int-BD2) (280 mg, 0.962 mmol) in aqueous HCl (6 M) (3 ml) was added sodium nitrite (80 mg, 1.155 mmol) in H₂O (2 ml) dropwise at −10° C. The mixture was stirred at -10° C. for 30 min. Then potassium iodide (288 mg, 1.732 mmol) in H₂O (2 ml) was added dropwise at −10° C. and then stirred at −10° C. for 10 min. The reaction mixture was extracted with EtOAc (8 mL×3), washed with Na₂SO₃ (10 mL), dried with Na₂SO₄, filtered and concentrated under vacuum. The crude product was purified by pre-TLC (Pet, ether/EtOAc=100:0) to give 4-bromo-2,5-dichloro-1-iodonaphthalene. ¹H NMR (500 MHz, CHLOROFORM-d) δ ppm 7.46 (t, J=8.01 Hz, 2H) 7.71-7.73 (m, 1H) 7.99 (s, 1H) 8.34 (dd, J=8.62, 0.99 Hz, 1H).

### STEP 4: 1-bromo-3,8-dichloronaphthalene (Int-BD4)

To a solution of 4-bromo-2,5-dichloro-1-iodonaphthalene (Int-BD3) (220 mg, 0.547 mmol) in THF (0.3 ml) was added mono(isopropylmagnesium(II)) monolithium monochloride hydrochloride (0.463 ml, 0.602 mmol) dropwise at -78° C. under N₂. The mixture was stirred at −78° C. for 2 h and methanol (52.6 mg, 1.642 mmol) in THF (0.3 ml) was stirred at −78° C. for 1 h. The reaction was warmed to rt, the mixture was concentrated, the residue was purified by pre-TLC ((Pet, ether/EtOAc=100:0)) to give 1-bromo-3,8-dichloronaphthalene.

### Intermediate BE1: 4-bromo-1,5-dichloro-2-(methoxymethoxy)naphthalene (Int-BE 1

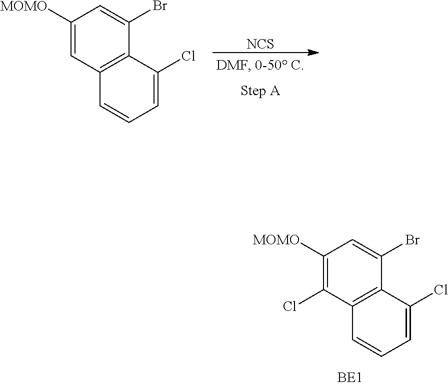

To a solution of 1-bromo-8-chloro-3-(methoxymethoxy)naphthalene (Int-III4) (150 mg, 0.497 mmol) in DMF (1.5 mL) was added NCS (86.0 mg, 0.647 mmol) at 0° C. in portions. The reaction mixture was warmed to 50° C. and stirred at 50° C. for 12 h. The mixture was quenched with H₂O (1 mL), extracted with EtOAc (3×2 mL), and dried over Na₂SO₄. The mixture was filtered and concentrated under reduced pressure to give the residue which was purified by flash silica gel chromatography 4 g column, Eluent of 0-5% EtOAc/Pet, ether gradient, dry loaded) to give 4-bromo-1,5-dichloro-2-(methoxymethoxy)naphthalene. ¹H NMR (400 MHz, CDCl₃) δ 8.31 (dd, J=1.17, 8.61 Hz, 1H), 7.92 (s, 1H), 7.60 (dd, J=1.17, 7.43 Hz, 1H), 7.37-7.51 (m, 1H), 5.35 (s, 2H), 3.58 (s, 3H).

### Intermediate BF5: 2-chloro-3-(methoxymethoxy)-8-((triisopropylsilyl)ethynyl)naphthalen-1-yltrifluoromethanesulfonate (Int-BF5)

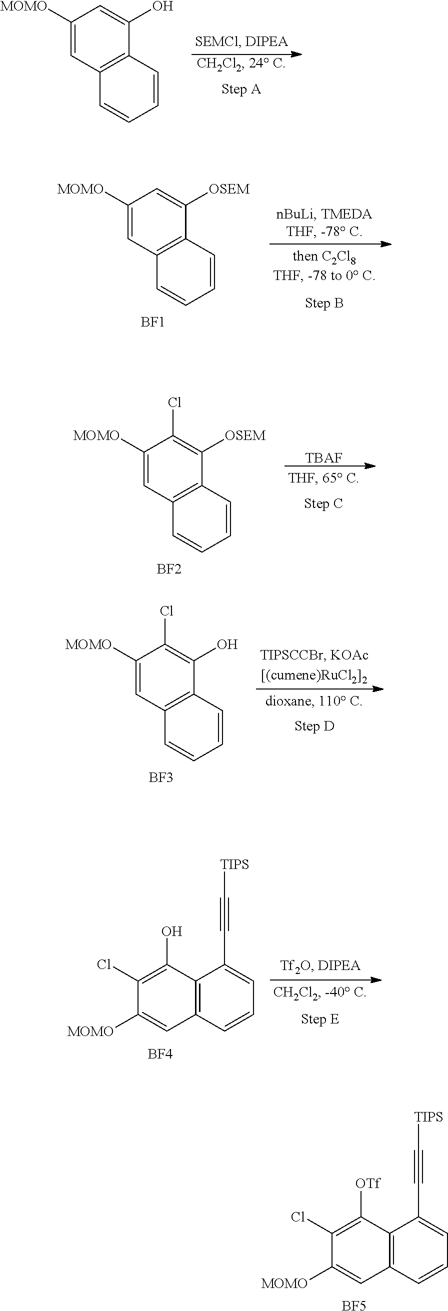

### Step A: (2-(((3-(methoxymethoxy)naphthalen-1-yl)oxy)methoxy)ethyl) trimethylsilane (Int-BF1)

3-(Methoxymethyl)naphthalen-1-ol (500 mg, 2.45 mmol) was dissolved in DCM (8.2 mL), then N-ethyl-N-isopropylpropan-2-amine (DIPEA) (1.74 μl, 9.79 mmol) and (2-(chloromethoxy)ethyl)trimethylsilane (SEMCI) (867 gL. 4.90 mmol) were added into the reaction vessel. The resulting mixture was stirred for 16h at 24° C. The product mixture was diluted with ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 20% ethyl acetate-hexanes, linear gradient to afford (2-(((3-(methoxymethoxy)naphthalen-1-yl)oxy)methoxy)ethyl)trimethylsilane. ¹H NMR (500 MHz, Chloroform-d) δ 8.17 (d, J=8.4 Hz, 1H), 7.71 (d, J=8.2 Hz, 1H), 7.46 (ddd, J=8.1, 6.8, 1.3 Hz, 1H), 7.43-7.32 (m, 1H), 7.06 (d, J=2.1 Hz, 1H), 6.89 (d. =2.1 Hz, 1H), 5.44 (s, 2H), 5.30 (d, J=1.2 Hz, 2H), 4.03-3.79 (m, 2H), 3.55 (d, J=1.2 Hz, 3H), 1.06-0.85 (m, 2H), 0.02 (d, J=1.2 Hz, 9H).

### Step B: (2-(((2-chloro-3-(methoxymethoxy)naphthalen-1-yl)oxy)methoxy) ethyl)trimethylsilane (Int-BF2)

(2-(((3-(methoxymethoxy)naphthalen-1-yl)oxy)methoxy)ethyl)trimethylsilane (Int-BF1) (820 mg, 2.45 mmol) was added into a 20 mL vial. The reaction vessel was evacuated and backfilled with a balloon of nitrogen three times. Tetrahydrofuran (6.1 mL) and TMEDA (441 μL, 2.94 mmol) were added into the reaction vessel. The resulting mixture was cooled down to −78° C. n-Butyl)ithium (1.18 mL, 2.94 mmol) was added dropwise into the reaction vessel. The resulting mixture was stirred for 15 mm at −78° C. then warmed up to 0° C. and stirred for 1 h. The resulting mixture was cooled back down to −78° C. then perchloroethane (6% mg, 2.94 mmol) in THF (2.0 mL) was added into the reaction vessel and the resulting mixture was warmed up to room temperature. The product mixture was quenched with saturated sodium bicarbonate aqueous solution (1 mL). The quenched product mixture was extracted three times with DCM (3×30 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford (2-(((2-chloro-3-(methoxymethoxy)naphthalen-1-yl)oxy)methoxy)ethyl)trimethylsilane. ¹H NMR (500 MHz, Acetonitrile-d3) δ 8.11 (d, J=8.4 Hz, 1H), 7.83 (d, J=8.1 Hz, 1H), 7.53 (t.. =7.5 Hz, 1H), 7.48 (t, J=7.6 Hz, 1H), 7.41 (s, 1H), 5.40 (d.. =1.5 Hz, 2H), 5.35 (d, J=1.5 Hz, 2H), 4.09-3.93 (m, 2H), 3.53 (d, J=1.4 Hz, 3H), 0.98 (dd, J=9.1, 7.6 Hz, 2H), 0.03 (d, J=1.5 Hz, 9H).

### Step C: 2-chloro-3-(methoxymethoxy)naphthalen-1-ol (Int-BF3)

(2-(((2-Chloro-3-(methoxymethoxy) naphthalen-1-yl)oxy)methoxy)ethyl)trimethylsilane (Int-BF2) (850 mg, 2.30 mmol) was added in a 100 mL round-bottomed flask equipped with a reflux condensor. The reaction vessel was evacuated and backfilled with a balloon of nitrogen three times. THF (4.6 mL) and tetrabutylammonium fluoride (1 M in THF, 23.0 mL, 23.0 mmol) were added into the reaction vessel. The resulting mixture was heated at 65° C. for 1 h. The product mixture was cooled to room temperature. The product mixture was diluted with ether (200 mL). The diluted product mixture was washed three times with saturated ammonium chloride aqueous solution (3-20 mL). The organic layer was dried over sodium sulfate. The dried solution was filtered and the filtrate was concentrated to dryness. The residue obtained was purified by flash column chromatography with 12-g silica gel column, eluting with hexanes initially, grading to 30% ethyl acetate-hexanes, linear gradient to afford 2-chloro-3-(methoxymethoxy)naphthalen-1-ol. ¹H NMR (499 MHz, Acetonitrile-d3) δ 8.11 (d, J=8.4 Hz, 1H), 7.76 (d, J=8.2 Hz, 1H), 7.51 (t, J=7.6 Hz, 1H), 7.47-7.28 (m, 2H), 7.15 (s, 1H), 5.38 (d, J=1.5 Hz, 2H), 3.53 (d, J=1.4 Hz, 3H).

### Step D: 2-chloro-3-(methoxymethoxy)-8-(triisopropylsilyl)ethynyl)naphthalen-1-ol (Int-BF4)

To 2-chloro-3-(methoxymethoxy)naphthalen-1-ol (Int-BF3) (390 mg, 1.63 mmol) and dichloro(p-cymene)ruthenium(II) dimer (100 mg, 0.163 mmol) and potassium acetate (321 mg, 3.27 mmol) in dioxane (2.7 mL), (bromoethynyl)triisopropylsilane (470 μL, 1.96 mmol) was added. The flask was thoroughly flushed with nitrogen and the reaction mixture was stirred for 16h at 110° C. The reaction mix was filtered through a bed of CELITE. The filter bed was washed with excess DCM. The filtrate was concentrated and directly purified on a silica gel column using 0-10% Hex- EtOAc to afford 2-chloro-3-(methoxymethoxy)-8-((triisopropylsilyl)ethynyl)naphthalen-1-ol. ¹H NMR (500 MHz, Acetonitrile-d3) δ 9.46 (s, 1H), 7.83 (d, J=8.3 Hz, 1H), 7.64 (d, J=7.2 Hz, 1H), 7.44 (t, J=7.8 Hz, 1H), 7.20 (s, 1H), 5.39 (s, 2H), 3.52 (d, J=1.1 Hz, 3H), 1.33-1.04 (m, 21H).

### Step E: 2-chloro-3-(methoxymethoxy)-8-((triisopropylsilyl)ethynyl)naphthalen--yltrifluoromethanesulfonate (Int-BF5)

To 2-chloro-3-(methoxymethoxy)-8-((triisopropylsilyl)ethynyl)naphthalen-1-ol (Int-BF4) (764 mg, 1.82 mmol) and DIPEA (956 μL, 5.47 mmol) in DCM (9.1 mL), triflic anhydride (462 μL, 2.74 mmol) was added at −40° C. and stirred for an hour. The reaction mixture was filtered through a bed of CELITE. The filter bed was washed with excess DCM. The filtrate was concentrated and directly purified on a silica gel column using 0-10% Hex- EtOAc to afford 2-chloro-3-(methoxymethoxy)-8-((triisopropylsilyl)ethynyl)naphthalen-1-yltrifluoromethanesulfonate. ¹H NMR (600 MHz, Acetonitrile-d₃) δ 7.92 (d, J=8.3 Hz, 1H), 7.81 (dd, J=7.3, 1.1 Hz, 1H), 7.72 (s, 1H), 7.56 (dd, J=8.3, 7.2 Hz, 1H), 5.45 (s, 2H), 3.53 (s, 3H), 1.26-1.17 (m, 21H).

### Intermediate BG5: 2.7-difluoro-8-((triisoprouylsilyl)ethynyl)naphthalen-1-yl trifluoromethanesulfonate (Int-BG5)

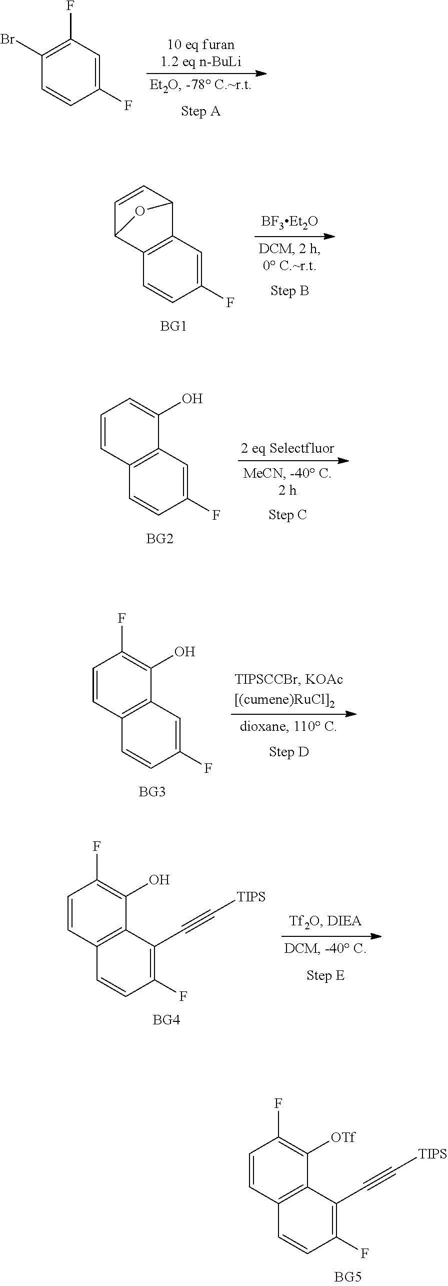

### STEP A: 6-fluoro-1.4-dihydro-1.4-epoxynaphthalene (Int-BG1)

To a solution of 1-bromo-2,4-difluorobenzene (11.70 mL, 104 mmol) in ethyl ether (700 g) was added n-butylthium (49.7 mL, 124 mmol) (2.5M in hexane) dropwise at −78° C. The reaction mixture was stirred at −78° C. for 35 min. Then furan (75 mL, 1036 mmol) was added dropwise to the above mixture at −78° C. The mixture was stirred at 20° C. for 15 h. The reaction mixture was quenched with aqueous NH₄Cl (50 mL), then the mixture was dissolved in water (50 mL), filtered and separated. The aqueous layer was extracted with EtOAc (150 mL×2), and the combined organic layers were dried over MgSO₄, filtered and concentrated in vacuum. The residue was purified by flash silica gel chromatography 80 g column, Eluent of 0-10% ethyl acetate/Pet, ether gradient to give 6-fluoro-1,4-dihydro-1.4-epoxynaphthalene. MS (ESI), m z (M+H)⁺ 163.

### Step B: 7-fluoronaphthalen-1-ol (Int-BG2)

Boron trifluoride diethyl etherate (7.35 g, 51.8 mmol) was added dropwise to a solution of 6-fluoro-1,4-dihydro-1,4-epoxynaphthalene (Int-BGI) (8 g, 49.3 mmol) in dry DCM (80 mL) at 0° C. After this addition (30 min) was finished, the mixture was allowed to warm to 30° C. and stirred for additional 2 h. The resulting solution (dark brown) was washed with water (20 mL×3). The organic layer was separated, dried with anhydrous Na₂SO₄, filtered and the solvent was evaporated under reduced pressure to yield a dark orange crystalline mass. The crude product was purified by flash silica gel chromatography 40 g column, Eluent of 0-5% EtOAc/Pet, ether gradient to give 7-fluoronaphthalen-1-ol. ¹H NMR (400 MHz, Acetone) δ 9.11 (s, 1H), 7.92-7.88 (dd, J=4.4, 7.2 Hz, 1H), 7.80 (dd, J=2.6, 10.7 Hz, 1H), 7.41-7.43 (d, J=6.8 Hz, 1H), 7.32-7.27 (m, 2H), 6.97-6.96 (d, J=6.0 Hz, 1H). Step C: 2.7-difluoronaphthalen-1-ol (Int-BG3)

To a solution of 7-fluoronaphthalen-1-ol (Int-BG2) (4.5 g, 27.7 mmol) in MeCN (50 mL) was added a solution of 1-chloromethyl-4-fluoro-1,4-diazoniabicyclo[2.2.2]octane bis(tetrafluoroborate) (9.83 g, 27.7 mmol) in MeCN (50 mL) at −40° C. After stirring at 40° C. for 2 h, the mixture was concentrated in vacuum. The crude product was purified by flash silica gel chromatography 80 g column, Eluent of 0-5% EtOAc/Pet, ether gradient to give 2,7-difluoronaphthalen-1-ol. ¹H NMR (400 MHz, Acetone) δ 9.08 (d, J=2.1 Hz, 1H), 7.81 (dd, J=5.7, 9.1 Hz, 1H), 7.69 (dd, J=2.6, 10.7 Hz, 1H), 7.35 (dd, J=4.8, 9.0 Hz, 1H), 7.22-7.13 (m, 2H).

### Step D: 2.7-difluoro-8-((triisopropylsilyl)ethynyl)naphthalen-1-ol (Int-BG4)

To a solution of 2,7-difluoronaphthalen-1-ol (Int-BG3) (600 mg, 3.33 mmol) in dioxane (8.0 mL) was added (bromoethynyl)triisopropylsilane (1044 mg, 4.00 mmol), potassium acetate (654 mg, 6.66 mmol) and dichloro(p-cymene)ruthenium(II) dimer (204 mg, 0.333 mmol) at 25° C. under N2 atmosphere, and the mixture was stirred at 110° C. for 16 h. TLC (Pet, ether/EtOAc=50/1) showed the starting material was consumed and new spots was formed. The reaction mixture was diluted with EtOAc (20 mL) and the resulting mixture was filtered. The filtrate was concentrated in vacuo to give the crude product, which was purified by flash silica gel chromatography 12 g column, Pet, ether/EtOAc=60/1, 30 min, dry loaded to give 2,7-difluoro-8-((triisopropylsilyl)ethynyl)naphthalen-1-ol (550 mg, 1.449 mmol, 43.5% yield) as yellow oil. MS (ESI): m/z (M+H)⁺ 361.

### Step E: 2.7-difluoro-8-((triisopropylsilyl)ethynyl)naphthalen-1-yl trifluoromethanesulfonate (Int-BG5)

To a solution of 2,7-difluoro-8-((triisopropylsilyl)ethynyl)naphthalen-1-ol (Int-BG4) (100 mg, 0.277 mmol) in DCM (2.0 mL) was added DIEA (0.145 mL. 0.832 mmol) and Tf₂O (0.070 mL, 0.416 mmol) at −40° C., and the mixture was stirred at −40° C. for 2 min. The reaction mixture was concentrated in vacuo, and the residue was purified by pre-TLC (Pet, ether/EtOAc=10:1) to give 2,7-difluoro-8-((triisopropylsilyl)ethynyl)naphthalen-1-yl trifluoromethanesulfonate.

¹H NMR (400 MHz, CDCl₃) δ 7.83-7.70 (m, 2H), 7.28 (td, J=9.0, 13.3 Hz. 2H), 1.21-1.07 (m, 21H).

### Intermediate BH7: 4-bromo-6-methyl-1-(tetrahydro-2H-pyran-2-yl)-5-(trifluoromethoxy)-1H-indazole (Int-BH7)

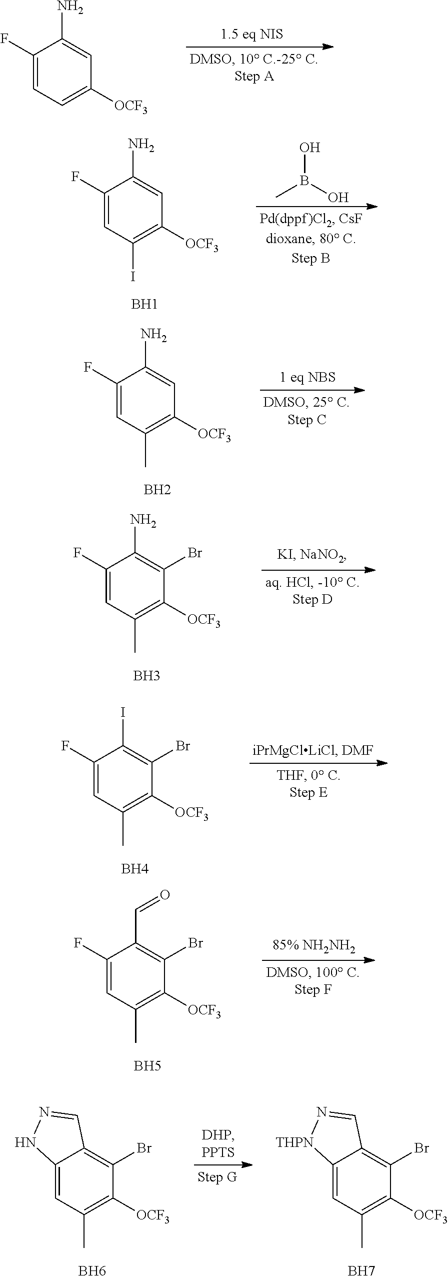

### Step A: 2-fluoro-4-iodo-5-(trifluoromethoxy)aniline (Int-BH1)

To a mixture of 2-fluoro-5-(trifluoromethoxy)aniline (3 g, 15.38 mmol) in DMSO (30 mL) was added N1S (5.19 g, 23.06 mmol) at 10° C. The mixture was stirred at 25° C. for 24 h. LCMS showed most of the starting material was consumed and desired MS was observed. The mixture was diluted with EtOAc (150 mL), washed with brine (4×20 mL), dried over Na₂SO₄, filtered and the solvent was evaporated under reduced pressure to give 2-fluoro-4-iodo-5-(trifluoromethoxy)aniline, which was used directly in the next step without further purification. MS (ESI): m/z (M+H)⁺ 321. ¹H NMR (400 MHz, CDCl₃) δ 7.37 (d, J=9.8 Hz, 1H), 6.73 (dd, J=1.2, 7.8 Hz, 1H), 4.05-3.81 (s, 2H). Step B: 2-fluoro-4-methyl-5-(trifluoromethoxy)aniline (Int-BH2)

To a mixture of 2-fluoro-4-iodo-5-(trifluoromethoxy)aniline (Int-BH1) (4.94 g, 15.4 mmol) in dioxane (50 mL) was added CsF (8.18 g, 53.9 mmol), methylboronic acid (2.76 g, 46.2 mmol) and PdCl₂(dppf) (0.563 g, 0.769 mmol) at 25° C. The mixture was stirred at 80° C. for 16 h under nitrogen atmosphere. LCMS showed the reaction was finished. The reaction mixture was diluted with EtOAc (100 mL), washed with H₂O (20 mL), brine (30 mL), then dried and concentrated under vacuum. The crude product was purified by flash silica gel chromatography 24 g column, Eluent of Pet, ether gradient, (dry load) to give 2-fluoro-4-methyl-5-(trifluoromethoxy)aniline. MS (ESI): m/z (M+H)⁺ 210. ¹H NMR (400 MHz, CDCl₃) δ 6.85 (d, J=11.0 Hz, 1H), 6.65 (dd, J=0.8, 7.8 Hz, 1H), 3.72 (s, 2H), 2.19 (s, 3H).

### Step C: 2-bromo-6-fluoro-4-methyl-3-(trifluoromethoxy)aniline (Int-BH3)

To a mixture of 2-fluoro-4-methyl-5-(trifluoromethoxy)aniline (Int-BH2) (2.4 g, 11.48 mmol) in DMSO (30 mL) was added 1-bromopyrrolidine-2,5-dione (NBS) (2.042 g, 11.48 mmol). The mixture was stirred at 25° C. for 10 min. LCMS showed the reaction was finished. The reaction mixture was diluted with 100 mL of EtOAc and washed with Na₂SO₃ (10 mL), brine (3×20 mL), the organic layer was dried with Na₂SO₄ and concentrated under vacuum. The crude product was purified by flash silica gel chromatography 24 g column. Eluent of Pet, ether gradient, (dry load) to give 2-bromo-6-fluoro-4-methyl-3-(trifluoromethoxy)aniline. MS (ESI): m/z (M+H+41)⁺328, 330. ¹H NMR (400 MHz, CDCl₃) δ 6.87 (d, J=11.0 Hz, 1H), 4.08 (br s, 2H), 2.27 (s, 3H).

### Step D: 3-bromo-1-fluoro-2-iodo-5-methyl-4-(trifluoromethoxy) benzene (Int-BH4)

To a mixture of 2-bromo-6-fluoro-4-methyl-3-(trifluoromethoxy)aniline (Int-BH3) (2.2 g, 7.64 mmol) in aqueous HCl (6 M) (20 mL) was added sodium nitrite (0.632 g, 9.17 mmol) in H₂O (10 mL) dropwise at −10° C. The mixture was stirred at −10° C. for 30 min. Then potassium iodide (1.902 g, 11.46 mmol) in H₂O (5 mL) was added dropwise at the same temperature and then stirred at -10° C. for 10 min. TLC showed the starting material was consumed and new spot formed. The reaction mixture was diluted with EtOAc (60 mL), then washed with Na₂SO₃ (aq., 10 mL), brine (20 mL×2), dried with Na₂SO₄ and concentrated under vacuum. The crude product was purified by flash silica gel chromatography 40 g column, Eluent of Pet, ether gradient, dry load) to give 3-bromo-1-fluoro-2-iodo-5-methyl-4-(trifluoromethoxy)benzene). 1H NMR (400 MHz, CDCl₃) δ 7.01 (d, J=7.8 Hz, 1H), 2.37 (s, 3H).

### Step E: 2-bromo-6-fluoro-4-methyl-3-(trifluoromethoxy) benzaldehyde (Int-BH5)

To a solution of 3-bromo-1-fluoro-2-iodo-5-methyl-4-(trifluoromethoxy)benzene (Int-BH4) (1 g, 2.507 mmol) in THF (15 mL) was added iPrMgCl-LiCl (2.121 mL, 2.76 mmol) (1,3 M in THF) at 0° C. under N2 atmosphere, the mixture was stirred at 0° C. for 20 min. Then a mixture of DMF (0.388 mL, 5.01 mmol) in THF (I mL) was added to the reaction at 0° C., the mixture was stirred at 0° C. for 10 min. TLC showed the starting material was consumed and a new spot was formed. The reaction was quenched with NH₄Cl (aq., 5 mL), diluted with 50 mL of EtOAc, then washed with brine (15 mL), the organic layer was dried over sodium sulfate and concentrated. The crude product was purified by flash silica gel chromatography 12 g column, Eluent of Pet, ether gradient, dry load) to give 2-bromo-6-fluoro-4-methyl-3-(trifluoromethoxy)benzaldehyde. ¹H NMR (400 MHz, CDCl₃) δ 10.31 (s, 1H), 7.09 (d, J=10.6 Hz, 1H), 2.46 (s, 3H).

### Step F: 4-bromo-6-methyl-5-(trifluoromethoxy)-1H-indazole (Int-BH6)

To a solution of 2-bromo-6-fluoro-4-methyl-3-(trifluoromethoxy)benzaldehyde (Int-BH5) (320 mg, 1.063 mmol) in DMSO (3 mL) was added N₂H₄ (0.196 mL, 5.32 mmol) (85% in water), and the mixture was stirred at 100° C. for 16 h. LCMS showed desired MS was observed and the starting material was consumed, the reaction mixture was diluted with EtOAc (20 mL), washed with brine (3 mL×3), dried and concentrated under vacuum. The crude product was purified by flash silica gel chromatography 4 g column, Eluent of 0-15% EtOAc/Pet, ether gradient, dry load) to give 4-bromo-6-methyl-5-(trifluoromethoxy)-1H-indazole. MS (ESI): m/z (M+H)⁺ 295, 297. ¹H NMR (400 MHz, CDCl₃) δ 8.12 (s, 1H), 7.34 (s, 1H), 2.51 (s, 3H).

### Step G: 4-bromo-6-methyl-1-(tetrahydro-2H-pyran-2-yl)-5-(trifluoromethoxy)-1H-indazole (Int-BH7)

To a solution of 4-bromo-6-methyl-5-(trifluoromethoxy)-1H-indazole (Int-BH6) (190 mg, 0.644 mmol) in THF (5 mL) was added 3.4-dihydro-2H-pyran (0.117 mL, 1.288 mmol), PPTS (32.4 mg, 0.129 mmol) at 25° C., and the mixture was stirred at 50° C. for 8 h. TLC (SiO₂, Pet, ether/EtOAc=5:1, v/v) showed starting material was consumed and a new spot formed. The mixture was concentrated under reduced pressure, the residue was purified by flash silica gel chromatography 4 g Column, Eluent of 15% ethyl acetate in petroleum ether gradient to give 4-bromo-6-methyl-2-(tetrahydro-2H-pyran-2-yl)-5-(trifluoromethoxy)-2H-indazole (Pt) and 4-bromo-6-methyl-1-(tetrahydro-2H-pyran-2-yl)-5-(trifluoromethoxy)-1H-indazole (P2). P2 was used for the further steps. MS (ESI): m/z (M+H)⁺ 378, 380. P1: ¹H NMR (400 MHz, CDCl₃) δ 8.02 (s, 1H), 7.42 (s, 1H), 5.68 (dd, 1=2.7, 9.0 Hz, 1H), 4.07-3.97 (m, 1H), 3.80-3.72 (m, 1H), 2.51 (s, 3H), 2.21-2.14 (m, 1H), 2.12-2.06 (m, 1H), 1.93-1.81 (m, 1H), 1.80-1.71 (m, 3H). P2: ¹H NMR (400 MHz, CDCl₃) δ 8.21 (s, 1H), 7.51 (s, 1H), 5.66 (dd, J=2.7, 9.0 Hz, 1H), 4.15 (br d, J=10.2 Hz, 1H), 3.83-3.76 (m, 1H), 2.51 (br s, 1H), 2.46 (s, 3H), 2.30-2.14 (m, 2H), 2.06 (br s, 1H), 1.79 (br d, J=9.0 Hz, 2H).

### Intermediate BI3: N-(1-bromo-8-chloroisoquinolin-3-yl)acetamide (Int-BI3)

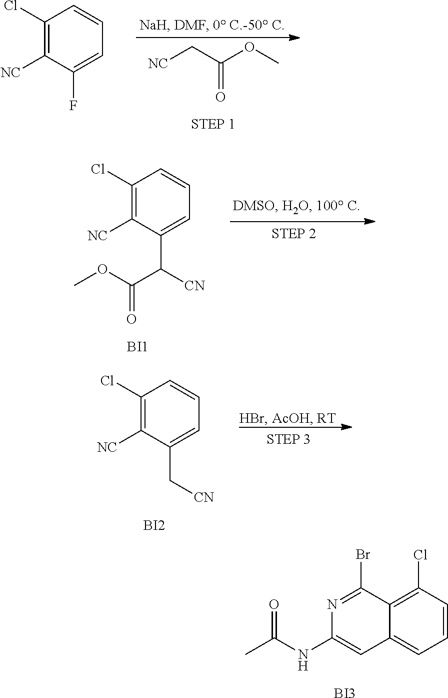

### STEP 1: Methyl 2-(3-chloro-2-cyanophenyl)-2-cyanoacetate (Int-BI1)

To a vial was added Sodium hydride (2.057 g, 51.4 mmol) and it was cooled to 0° C. A mixture of methyl 2-cyanoacetate (5.10 g, 51.4 mmol)) in DMF (15 ml)) was added to the flask dropwise. The mixture was allowed to warm to room temperature for one hour. After 1 hour, 2-chloro-6-fluorobenzonitrile (4 g. 25.7 mmol) in DMF (15 ml)) was added. The mixture was then heated to 50° C. for 4 hours. Upon cooling to room temperature, the mixture was carefully quenched with 2N HCl and then ethyl acetate was added. The organic layer was then separated, dried over magnesium sulfate, filtered and concentrated under reduced pressure to yield methyl 2-(3-chloro-2-cyanophenyl)-2-cyanoacetate. MS (ESI): m/z (M+H+Na)⁺ 257.

### STEP 2: 2-chloro-6-(cyanomethyl)benzonitrile (Int-B₁₂)

To a vial containing methyl 2-(3-chloro-2-cyanophenyl)-2-cyanoacetate (Int-BI1) (550 mg, 2.344 mmol) was added deuterated DMSO (3.8 ml) and then Water (0.5 ml). The mixture was heated to 100° C. for 18 hrs. The mixture was allowed to cool to room temperature and quenched with water. The resulting solid was filtered and then washed with water. The solids were then dried under vacuum to yield the desired product 2-chloro-6-(cyanomethyl)benzonitrile. MS (ESI): m/z (M+H+Na)⁺199.

### STEP 3: N-(1-bromo-8-chloroisoquinolin-3-yl)acetamide (Int-B₁₃)

HBr in 33% acetic acid (3 ml) was added to 2-chloro-6-(cyanomethyl)benzonitrile (Int-BI2) (400 mg, 2.265 mmol) and stirred at rt for overnight. The reaction mixture was slowly quenched with sat. NaHCO₃ solution with stirring and the solid precipitated was filtered and dried under vacuum. The resulting solid material was washed with diethyl ether and dried under vacuum to yield N-(1-bromo-8-chloroisoquinolin-3-yl)acetamide. MS (ESI): m z (M+H)⁺ 299.

### Intermediate BJ3:3-bromo-2-fluoro-N,N-bis(4-methoxybenzyl))-5-methyl-4-(trifluoromethyl)aniline (Int-BJ=3)

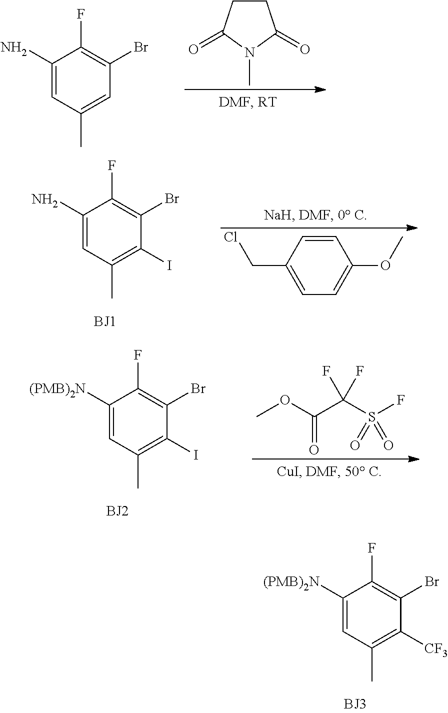

### STEP 1: 3-bromo-2-fluoro-4-iodo-5-methylaniline (Int-BJ=1)

To a solution of 3-bromo-2-fluoro-5-methylaniline (2000 mg, 9.80 mmol) in DMF (48 ml) was added 1-iodopyrrolidine-2,5-dione (2646 mg, 11.76 mmol). The reaction mixture was stirred at rt for 4 hrs. It was then quenched with water and extracted with EtOAC. The organic layers were combined, washed with water, brine solution and then concentrated under reduced pressure. The residue was purified by 0-15% of EtOAc in hexane to yield 3-bromo-2-fluoro-4-iodo-5-methylaniline. MS (ESI): m/z (M+H)⁺ 330.

### STEP 2: 3-bromo-2-fluoro-4-iodo-N,N-bis(4-methoxybenzyl)-5-methylaniline (Int-BJ=2)

To a solution of 3-bromo-2-fluoro-4-iodo-5-methylaniline (Int-BJ=1) (1400 mg, 4.24 mmol) in DMF (21 ml) was added sodium hydride (509 mg, 12.73 mmol) under nitrogen at 0° C. The reaction mixture was stirred at the same temperature for 1 hr and was added 1-(chloromethyl)-4-methoxybenzene (1.156 ml, 8.49 mmol). The resulting mixture was stirred at rt for 1 hr. It was then quenched with sat NH₄Cl and extracted with EtOAc. The combined organic layers were washed with water, brine solution and concentrated. The product was purified by 0-100% of EtOAc in hexane to yield 3-bromo-2-fluoro-4-iodo-N,N-bis(4-methoxybenzyl)-5-methylaniline. The product has poor ionization in LCMS.

### STEP 3: 3-bromo-2-fluoro-N,N-bis(4-methoxybenzyl)-5-methyl-4-(trifluoromethyl)aniline (Int-BJ=3)

To a solution of 3-bromo-2-fluoro-4-iodo-N,N-bis(4-methoxybenzyl)-5-methylaniline (Int-BJ=2) (932 mg, 1.634 mmol) in DMF (9320 μl) was added Copper(I) iodide (934 mg, 4.90 mmol) followed by methyl 2,2-difluoro-2-(fluorosulfonyl)acetate (416 μl 3.27 mmol). The reaction mixture was heated at 50° C. for 2 hr and then quenched with sat. NH₄Cl solution and extracted with EtOAc. The combined organic layers were washed with water, brine solution and concentrated. The product was purified by silica gel column with 0-20% of EtOAc in hexane to yield 3-bromo-2-fluoro-N,N-bis(4-methoxybenzyl)-5-methyl-4-(trifluoromethyl)aniline. MS (ESI): m/z (M+H)⁺ 513.

### Intermediate BK3: N-(6-bromo-3-(difluoromethoxy)-4-methylpyridin-2-yl)

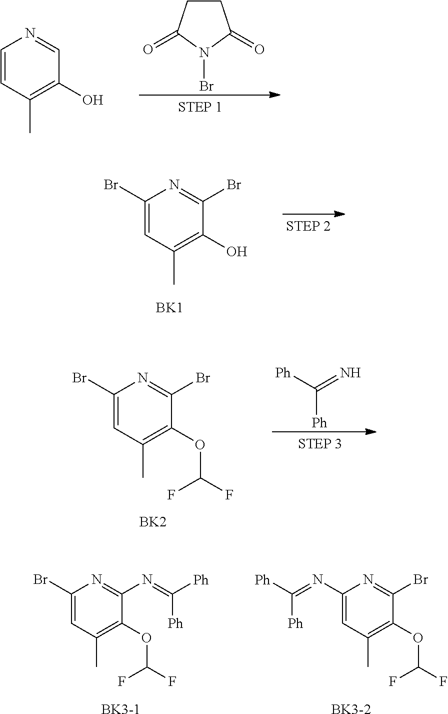

### STEP 1: 2,6-dibromo-4-methylpyridin-3-ol (Int-BK1)

To a solution of 4-methylpyridin-3-ol (2 g, 18.33 mmol) in MeCN (40 ml) was added NBS (6.52 g, 36.7 mmol) at 20° C. The mixture was stirred at 20° C. for 12 h. The reaction was monitored by LC. The mixture was diluted with water (20 mL), extracted with EtOAc (3×20 mL), dried over Na₂SO₄, filtered and the solvent was evaporated under reduced pressure to give the crude 2,6-dibromo-4-methylpyridin-3-ol as brown oil, which was used directly in next step without further purification. MS (EST) m/z: [M+H]⁺: 267.

### STEP 2: 2,6-dibromo-34difluoromethoxy)-4-methylpyridine (Int-BK2)

To a solution of 2,6-dibromo-4-methylpyridin-3-ol (Int-BK1) (4.7 g, 17.61 mmol) in DMF (80 ml) was added Cs₂CO₃ (11.47 g, 35.2 mmol) and sodiumchlorodifluoroacetate (4.03 g, 26.4 mmol) at 20° C. The mixture was stirred at 50° C. for 2 h. The reaction was monitored by LC. The mixture was cooled, diluted with water (30 mL), extracted with EtOAc (3×30 mL), dried over Na₂SO₄, filtered and the solvent was evaporated under reduced pressure to give the crude product. The crude residue was purified by flash silica gel chromatography 40 g silica column, Eluent of 0-20% EA/PE to give 2,6-dibromo-3-(difluoromethoxy)-4-methylpyridine. MS (EST) m/z: [M+H]⁺: 317.

### STEP 3: N-(6-bromo-3-(difluoromethoxy)-4-methylpyridin-2-yl)-1,1-diphenylmethanimine (Int-BK3-1)

A mixture of 2,6-dibromo-3-(difluoromethoxy)-4-methylpyridine (Int-BK2) (200 mg, 0.631 mmol), diphenylmethanimine (126 mg, 0.694 mmol), sodium 2-methylpropan-2-olate (121 mg, 1.262 mmol), Pd₂dba₃ (28.9 mg, 0.032 mmol), di-tert-butyl(2′,4′,6′-triisopropyl-[1.1′-biphenyl]-2-yl)phosphane (26.8 mg, 0.063 mmol) in Toluene (5 ml) was stirred at 60° C. for 3 hr and the reaction was monitored by LC. The mixture was concentrated, diluted with water (8 mL), extracted with EtOAc (3×8 mL), dried over Na₂SO₄, filtered and the solvent was evaporated under reduced pressure to give the crude product. The residue was purified by reverse preparative HPLC (Column. Boston Prime C18 150*30 mm*5 um; Condition: water (0.05% NH₃H₂O+10 mM NH₄HCO₃)-ACN to give N-(6-bromo-3-(difluoromethoxy)-4-methylpyridin-2-yl)-1,1-diphenylmethanimine (Int-BK3-1) Peak 1 and N-(6-bromo-5-(difluoromethoxy)-4-methylpyridin-2-yl)-1.1-diphenylmethanimine as (Int-BK3-2) Peak 2. MS (ESI) m/z: [M+H]⁺: 417, 419.

### Intermediate BM4: Tert-butyl (1S,5R)-3-(7-(4-chloro-2-(methoxymethoxy)-5-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-BM4) (Isomer 1) and Tert-butyl (0S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-BM4) (Isomer 2)

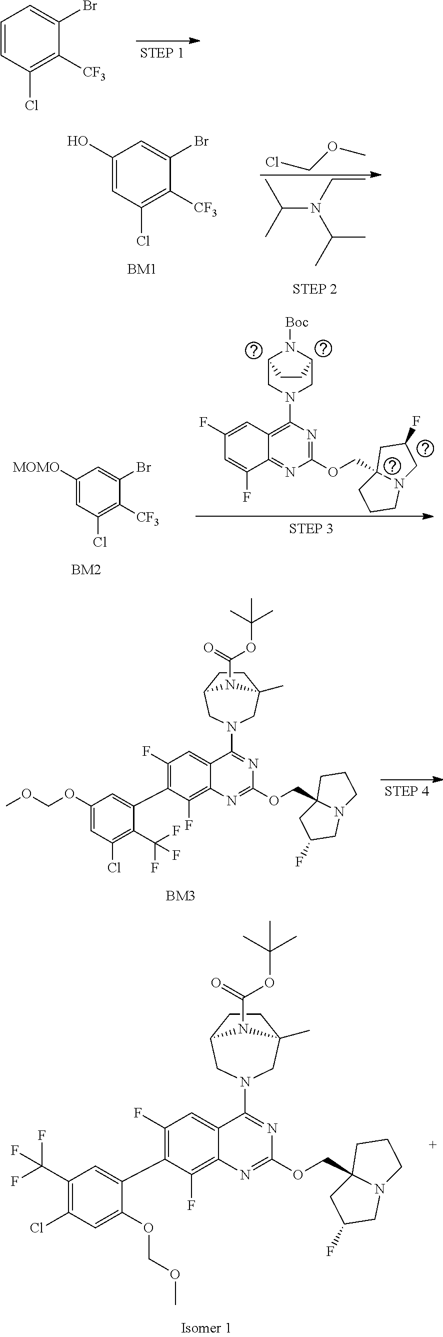

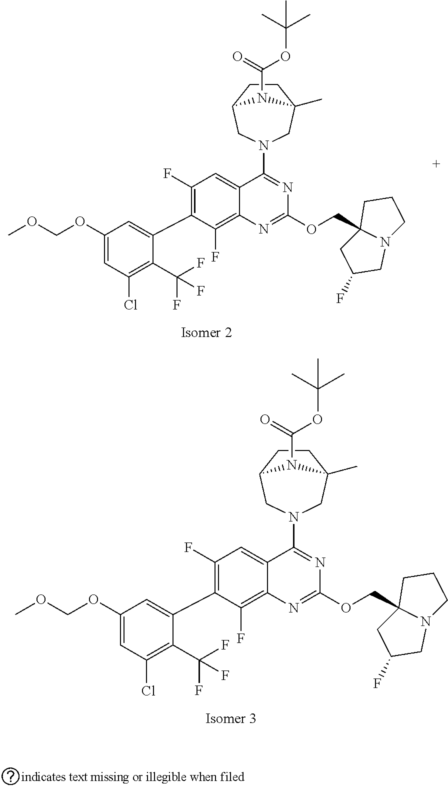 [SMILES: COCOc1cc(Cl)c(C(F)(F)F)c(-c2c(F)cc3c(N4C[C@H]5CC[C@@](C)(C4)N5C(=O)OC(C)(C)C)nc(OC[C@@]45CCCN4C[C@H](F)C5)nc3c2F)c1 ; COCOc1cc(Cl)c(C(F)(F)F)c(-c2c(F)cc3c(N4C[C@H]5CC[C@@](C)(C4)N5C(=O)OC(C)(C)C)nc(OC[C@@]45CCCN4C[C@H](F)C5)nc3c2F)c1]

### STEP 1: 3-bromo-5-chloro-4-(trifluoromethyl)phenol (Int-BM1)

To a solution of 1-bromo-3-chloro-2-(trifluoromethyl)benzene (4.5 g, 17.34 mmol) in THF (45 ml) was added 4,4′-di-tert-butyl-2,2′-bipyridine (0.559 g, 2.081 mmol), 4,4,5,5-tetramethyl-1,3,2-dioxaborolane (5.03 ml, 34.7 mmol) and bis(1,5-cyclooctadiene)dimethoxydiridium (0.345 g, 0.520 mmol) at 25° C. The mixture was stirred at 60° C. for 16 h. The mixture was concentrated to give a black oil which was dissolved in THF (100 ml) and H₂O (50 ml) was added. AcOH (39.7 ml, 694 mmol) and hydrogen peroxide (35.8 ml, 347 mmol) was additionally added to the mixture at 10° C. The reaction mixture was stirred at 10° C. for 1 h and monitored by TLC. The reaction mixture was quenched with water (1(0) mL), extracted with EtOAc (3×100 mL). The combined organic layer was washed with saturated aqueous Na₂SO₃ (3×100 mL) and brine (2×100 mL), dried over Na₂SO₄, filtered and the solvent was evaporated under reduced pressure to give crude. The crude product was purified by flash silica gel chromatography 80 g column. Eluent of 0-10% Pet, ether/EtOAc gradient, to give 3-bromo-5-chloro-4-(trifluoromethyl)phenol. ¹H NMR (400 MHz, CHLOROFORM-d) δ=7.16 (d, J=2.2 Hz, 1H), 6.97 (d, J=2.0 Hz, 1H), 6.24 (br s, 1H).

### STEP 2: 1-bromo-3-chloro-5-(methoxymethoxy)-2-(trifluoromethyl)benzene (Int-BM2)

To a solution of 3-bromo-5-chloro-4-(trifluoromethyl)phenol (Int-BM1) (4.1 g, 14.88 mmol) in DCM (30 ml) was added DIEA (9.10 ml, 52.1 mmol) and MOM-Cl (2.58 ml, 33.9 mmol) at 0° C. Then the reaction mixture was stirred at 25° C. for 1 h and monitored by TLC. The mixture was quenched with H₂O (50 mL), extracted with DCM (3×40 mL). The combined organic layer was washed with saturated aqueous brine (2×40 mL), dried over Na₂SO₄, filtered and the solvent was evaporated under reduced pressure to give the crude. The crude was purified by flash silica gel chromatography 40 g column, Eluent of 0-10% Pet, ether/EtOAc gradient, to give 1-bromo-3-chloro-5-(methoxymethoxy)-2-(trifluoromethyl)benzene. ¹H NMR (400 MHz, CHLOROFORM-d) δ=7.33 (d, J=2.0 Hz, 1H), 7.15 (d, 1=1.7 Hz, 1H), 5.20 (s, 2H), 3.49 (s, 3H).

### STEP 3: Tert-butyl (1S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-BM3)

To a solution of tert-butyl (1 S,5R)-3-(6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89a) (250 mg, 0.457 mmol) in THF (2 mL) was added (TMP)₂Zn₂MgCl₂-2LiCl (5.53 mL, 1.826 mmol) at 25° C. under N₂ atmosphere. The mixture was stirred at 50° C. for 4 h. After cooling to room temperate, a solution of CPHOS PD G3 (36.8 mg, 0.046 mmol) and 1-bromo-3-chloro-5-(methoxymethoxy)-2-(trifluoromethyl)benzene (Int-BM2) (438 mg, 1.370 mmol) in THF (2 mL) was added under N₂ atmosphere. The mixture was stirred at 50° C. for another 3 h. Reaction was monitored using LCMS, The mixture was quenched with H₂O (5 mL) and filtered, the filtrate was extracted with EtOAc (5 mL×3), the organic layers were washed with brine (20 mL), dried over anhydrous Na₂SO₄, filtered and concentrated. The residue was purified by flash silica gel chromatography Silica-CS(10 g), Eluent of 73% EA gradient, to give tert-butyl (1S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (contains 50% of tert-butyl (1S,5R)-3-(7-(4-chloro-2-(methoxymethoxy)-5-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate). MS (ESI): m/z (M+H)⁺ 786.

### STEP 4: tert-butyl (1S,5R)-3-(7-(4-chloro-2-(methoxymethoxy)-5-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate and tert-butyl (1S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-BM4)

Tert-butyl (1 S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-BM3) (40 mg, 0.051 mmol) (contains 50% of tert-butyl (1S,5R)-3-(7-(4-chloro-2-(methoxymethoxy)-5-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate) was separated by SFC (Column: Chiralcel 03-3 150ᵢÅ4.6 mm I.D., 3 um Mobile phase: A: CO₂ B:ethanol (0.05% DEA) Gradient: from 5% to 40% of B in 5 min and from 40% to 5% of B in 0.5 min, hold 5% of B for 1.5 min Flow rate: 2.5 mL/min Column temp.: 35° C.) to give Int-BM4 isomer 1 tert-butyl (1S,5R)-3-(7-(4-chloro-2-(methoxymethoxy)-5-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (RT=2.584 min, UV=220 nm), Int-BM4 isomer 2 tert-butyl (1S,5R)-3-(7-(3-chloro-5-(methoxymethoxy)-2-(trifluoromethyl)phenyl)-6,8-difluoro-2-(((2R, 7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (RT=2.824 min, UV=220 nm) and give Int-BM4 isomer 3 tert-butyl (1S,5R)-3-(7-(3-chloro-5-(methoxymethoxy)-2-(trifluoromethyl)phenyl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (RT=3.130 min, UV=220 nm). Isomer 1 and 2 was confirmed by 2DNMR. Isomer 1-MS (ESI) m/z: calc'd [M+H]⁺: 786; Isomer 2-MS (ESI) m/z: calc'd [M+H]⁺: 786; Isomer 3-MS (ESI) m/z: calc'd [M+H]⁺: 786.

### Intermediate BN7: 4-bromo-N,N-bis(2.4-dimethoxybenzyl)-5,7-difluorobenzo[d]oxazol-2-amine (Int-BN7)

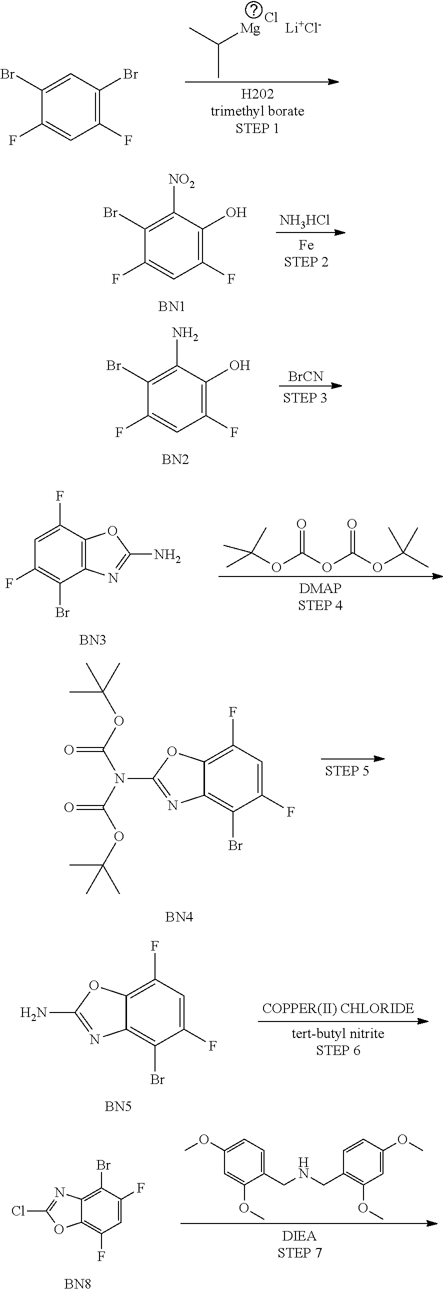

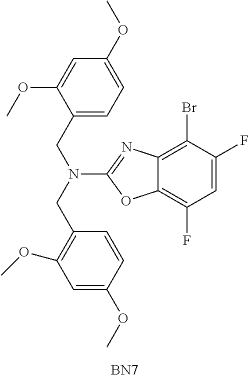 [SMILES: COc1ccc(CN(Cc2ccc(OC)cc2OC)c2nc3c(Br)c(F)cc(F)c3o2)c(OC)c1 ; "BN7"]

### STEP 1: 3-bromo-4,6-difluoro-2-nitrophenol (Int-BN1)

To a stirred solution of 1.5-dibromo-2,4-difluorobenzene (37 g, 136 mmol) in THF (200 ml) was added isopropylmagnesium chloride lithium chloride complex (115 mi, 150 mmol) (1,3 M in THF) at −20° C., and the mixture was stirred at 0° C. for 30 min under N₂ atmosphere. Then trimethyl borate (16.97 g, 163 mmol) was added and the mixture stirred at 25° C. for 1 hour, the solution was re-cooled to −20° C., H₂O₂(17.87 ml, 204 mmol) (35% solution) was added and the solution was stirred for 30 minutes at 25° C. The solution was then quenched with sodium bisulfite (5% solution, 40 mL) and then acidified with 6 N hydrochloric acid and extracted with EtOAc (2×50 mL). The combined organic layers were washed with brine (30 mL), dried over Na₂SO₄, filtered and the solvent was evaporated under reduced pressure. The residue was purified by flash silica gel chromatography 120 g column. Eluent of 0-5% EtOAc/Pet, ether gradient® 40 mL/min, dry loaded to give 3-bromo-4,6-difluoro-2-nitrophenol.

¹H NMR (400 MHz, CHLOROFORM-d) 5=7.32-7.12 (m, 1H), 7.04-6.86 (m, 1H), 5.94 (br s, 1H).

### STEP 2: 2-amino-3-bromo-4.6-difluoronhenol (Int-BN2)

To a stirred solution of 3-bromo-4,6-difluoro-2-nitrophenol (Int-BNI) (14 g, 55.1 mmol) in THF (60 ml) and ammonium chloride (39.4 ml, 276 mmol) (7 M) was added Iron (15.39 g, 276 mmol) at 25° C., and the reaction mixture was stirred at 35° C. for 16 h. The reaction mixture was diluted with EtOAc (200 ml), and the mixture was filtered. The filtrate cake was concentrated in vacuo, and the residue was purified by flash silica gel chromatography 120 g Column. Pet.ether/EtOAc=5/1, 30 min, 40 mL/min, dry loaded) to give 2-amino-3-bromo-4,6-difluorophenol. MS (ESI): m/z (M+H)⁺ 223, 225. ¹H NMR (400 MHz, DMSO-d₆) δ=9.46 (s, 1H), 6.56 (dd, J=9.3, 10.8 Hz, 1H), 5.29 (br s, 2H).

### STEP 3: 4-bromo-5,7-difluorobenzo[d]oxazol-2-amine (Int-BN3)

To a stirred solution of 2-amino-3-bromo-4,6-difluorophenol (Int-BN2) (5 g, 22.32 mmol) in MeOH (50 ml) and DCM (10.0 ml) was added cyanic bromide (4.310 g, 40.7 mmol) at 25° C., and the mixture was stirred at 50° C. for 16 h under N₂ atmosphere. The reaction was monitored by LC. The mixture was concentrated, and the residue was purified by flash silica gel chromatography 40 g column. Pet, ether/EtOAc=3/1, 30 min, 40 mL/min, dry loaded) to give 4-bromo-5,7-difluorobenzo[d]oxazol-2-amine. MS (ESI): m/z (M+H)⁺ 248, 250.

¹H NMR (400 MHz, DMSO-d₆) δ=8.11 (s, 2H), 7.13 (t, J=10.3 Hz, 1H).

### STEP 4: Tert-butyl (4-bromo-5,7-difluorobenzo[d]oxazol-2-yl)(tert-butoxycarbonylkarbamate (Int-BN4)

To a stirred solution of 4-bromo-5,7-difluorobenzo[d]oxazol-2-amine (Int-BN3) (1 g, 4.02 mmol) in DCM (10.0 ml) was added DMAP (0.049 g, 0.402 mmol) and Boc₂O (2,331 ml, 10.04 mmol) at 25° C., and the mixture was stirred at 25° C. for 2 h. The reaction was monitored by LC. The mixture was concentrated, and the residue was purified by flash silica gel chromatography 40 g Column, Pet, ether/EtOAc=3/l, 30 min, 40 mL/min, dry loaded) to give tert-butyl (4-bromo-5,7-difluorobenzo[d]oxazol-2-yl)(tert-butoxycarbonyl)carbamate. MS (ESI): m/z (M+H-Boc-tBu)*292, 294. ¹H NMR (400 MHz, DMSO-d₆) δ=7.83 (t, J=10.3 Hz, 1H), 1.42 (s, 18H).

### STEP 5: 4-bromo-5.7-difluorobenzo[d]oxazol-2-amine (Int-BN5)

A mixture of tert-butyl (4-bromo-5,7-difluorobenzo[d]oxazol-2-yl)(tert-butoxycarbonyl) carbamate (Int-BN4) (150 mg, 0.334 mmol) in DCM (1.5 ml) and TFA (0.5 ml) was stirred at 25° C. for h. The reaction was monitored by LC. The reaction mixture was concentrated in vacuo to give 4-bromo-5,7-difluorobenzo[d]oxazol-2-amine as yellow oil, which was used in the next step without further purification. MS (EST): m/z (M+H)⁺ 248, 250.

### STEP 6: 4-bromo-2-chloro-5,7-difluorobenzo[d]oxazole (Int-BN6)

To a stirred solution of 4-bromo-5.7-difluorobenzo[d]oxazol-2-amine (Int-BN5) (83 mg, 0.333 mmol) and copper (II) chloride (49.3 mg, 0.367 mmol) in MeCN (1 mL) was added tert-butyl nitrite (37.8 mg, 0.367 mmol) in MeCN (0.5 mL) at 0° C., and the mixture was stirred at 25° C. for 1 h. Reaction was monitored by LC, tert-butyl nitrite (37.8 mg, 0.367 mmol) was added to the reaction mixture, and the reaction mixture was stirred at 25° C. for 12 h. LCMS and TLC showed the starting material was consumed. The reaction was quenched with water (2 mL) and the resulting mixture was extracted with EtOAc (10 mL×3). The combined organic phase was dried over anhydrous Na₂SO₄, filtered and concentrated in vacuo. The residue was purified by preparative TLC plate (SiO₂, Pet.ether/EtOAc=15/1) to give 4-bromo-2-chloro-5,7-difluorobenzo[d]oxazole. ¹H NMR (400 MHz, METHANOL-d₄) δ=7.35 (t, J=9.9 Hz, 1H).

### STEP 7; 4-bromo-N,N-bis(2,4-dimethoxybenzyl)-5,7-difluorobenzo[d]oxazol-2-amine (Int-BN7)

To a stirred solution of 4-bromo-2-chloro-5,7-difluorobenzo[d]oxazole (Int-BN6) (20 mg, 0.075 mmol) in DMSO (1 mL) was added DIEA (0.039 mL, 0.224 mmol) and bis(2,4-dimethoxybenzyl)amine (28.4 mg, 0.089 mmol), and the resulting mixture was stirred at 120° C. for 12 h. The reaction was monitored by LC. The reaction mixture was diluted with EtOAc (20 ml) and water (5 ml), and the resulting mixture was washed with brine(5 ml×3). The combined organic phase was dried over anhydrous Na₂SO₄, filtered and concentrated in vacuo. The residue was purified by preparative TLC plate (SiO₂. Pet.ether/EtOAc=3/1) to give 4-bromo-N,N-bis(2,4-dimethoxybenzyl)-5,7-difluorobenzo[d]oxazol-2-amine. MS (ESI): m/z (M+H)⁺ 548, 550. ¹H NMR (400 MHz, METHANOL-da) 6=7.07 (br d, J=8.3 Hz, 2H), 6.81 (t, J=10.0 Hz, 1H), 6.48 (d, 1=2.2 Hz, 2H), 6.43 (dd, J=2.4, 8.3 Hz, 2H), 4.64 (s, 4H), 3.75 (s, 6H), 3.71 (s, 6H).

### Intermediate BO₁: 6-chloro-N,N-bis(4-methoxybenzyl)-5-trifluoromethyl)pyrazin-2-amine (Int-BO1)

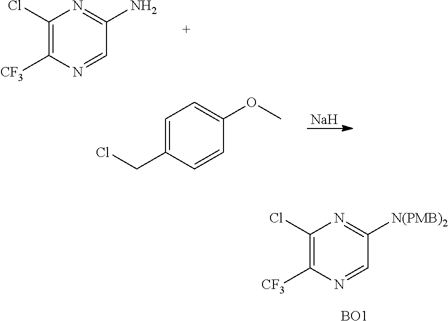

Sodium hydride (0.810 g, 20.25 mmol) was taken up in DMF (20.25 ml). 6-chloro-5-(trifluoromethyl)pyrazin-2-amine (1 g, 5.06 mmol) was added leading to gas evolution. 4-Methoxybenzyl chloride (2.75 ml, 20.25 mmol) was added dropwise and the reaction mixture was stirred at rt for 3 hours. Reaction mixture was diluted with water, extracted with EtOAc (3×), washed with brine, dried over Na₂SO₄ and concentrated under reduced pressure. The crude mixture was purified by silica gel column chromatography (0-50% EtOAc in hex) to afford 6-chloro-N,N-bis(4-methoxybenzyl)-5-(trifluoromethyl)pyrazin-2-amine. [M+H]⁺ Found: 438.

### Intermediate BP7: 4-chloro-N,N-bis(4-methoxybenzyl)-3,6-dimethyl-5-(trifluoromethyl)pyridin-2-amine (Int-BP7)

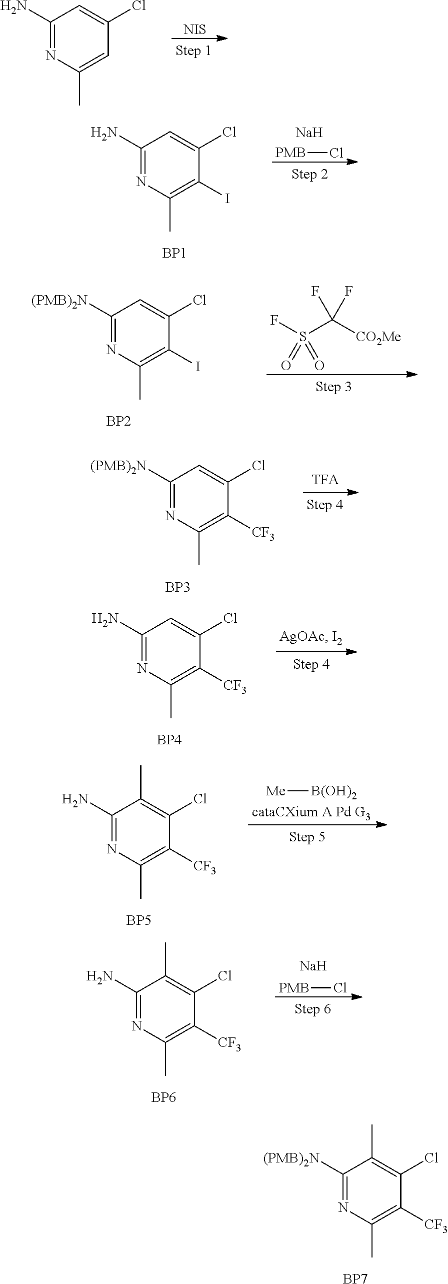

### Step 1: 4-chloro-5-iodo-6-methylpyridin-2-amine (Int-BPI)

A solution of 4-chloro-6-methylpyridin-2-amine (1 g, 7.01 mmol) and N1S (1.657 g, 7.36 mmol) in DMF (15 ml) was allowed to stir at rt for 72h. The reaction mixture was partitioned between sat. aqueous sodium thiosulfate/EtOAc. The phases were separated and the aqueous phase extracted with EtOAc. The combined organic layers were then washed with 10% aq. LiCl, dried over magnesium sulfate, and concentrated under reduced pressure. The residue was purified by flash chromatography over silica (0-50% EtOAc:hexanes) to afford 4-chloro-5-iodo-6-methylpyridin-2-amine. [M+H]⁺ Found: 269.

### Step 2: 4-chloro-5-iodo-N,N-bis(4-methoxybenzyl)-6-methylpyridin-2-amine (Int-BP2)

To a 0° C. cooled solution of 4-chloro-5-iodo-6-methylpyridin-2-amine (Int-BPI) (1.774 g, 6.61 mmol) in DMF (20 ml) was added sodium hydride (60% in mineral oil) (0.793 g, 19.82 mmol) and the resultant solution was allowed to stir at 0° C. for 1 h. To the reaction mixture, 4-methoxybenzyl chloride (1.800 ml, 13.22 mmol) was added and allowed to stir at 0° C. for 30 minutes. The reaction mixture was partitioned between EtOAc/water. The phases were separated and the aqueous phase extracted with EtOAc. The combined organic layers were then washed with 10% aqueous LiCl, dried over magnesium sulfate, and concentrated under reduced pressure. The residue was purified by flash chromatography over silica (0-50% EtOAc:hexanes) to afford 4-chloro-5-iodo-N,N-bis(4-methoxybenzyl)-6-methylpyridin-2-amine. [M+H]⁺ Found: 50).

### Step 3: 4-chloro-N,N-bis(4-methoxybenzyl)-6-methyl-5-(trifluoromethyl)pyridin-2-amine (Int-BP3)

To a solution of 4-chloro-5-iodo-N,N-bis(4-methoxybenzyl)-6-methylpyridin-2-amine (Int-BP2) (3.113g, 6.12 mmol) in DMF (30.6 ml) was added methyl 2,2-difluoro-2-(fluorosulfonyl)acetate (2,351 g, 12.24 mmol) and copper(l) iodide (3.50 g, 18.36 mmol). The resulting mixture was allowed to stir at 100° C. for 18h. The reaction mixture was partitioned between EtOAc/water. The phases were separated and the aqueous phase extracted with EtOAc. The combined organic layers were then washed with 10% aqueous LiCl, dried over magnesium sulfate, and concentrated under reduced pressure. The residue was purified by flash chromatography over silica (0-10% EtOAc:hexanes) to afford 4-chloro-N,N-bis(4-methoxybenzyl)-6-methyl-5-(trifluoromethyl)pyridin-2-amine. [M+H]⁺ Found: 451.

### Step 4: 4-chloro-6-methyl-5-(trifluoromethyl)pyridin-2-amine (Int-BP4)

To a solution of 4-chloro-N,N-bis(4-methoxybenzyl)-6-methyl-5-(trifluoromethyl)pyridin-2-amine (Int-BP3) (1.851g, 4.11 mmol) in trifluoroacetic acid (15 ml, 195 mmol) was added L-cysteine hydrochloride (0.776 g, 4.93 mmol). The resulting mixture was allowed to stir at 50° C. for 2h. The reaction mixture was partitioned between sat. aqueous NaHCO/EtOAc. The phases were separated and the aqueous phase extracted with EtOAc. The combined organic layers were then washed with brine, dried over magnesium sulfate, and concentrated under reduced pressure. The residue was purified by flash chromatography over silica (0-25% [3:1 EtOAc/EtOH]:hexanes) to afford 4-chloro-6-methyl-5-(trifluoromethyl)pyridin-2-amine. [M+H]⁺ Found: 211.

### Step 5: 4-chloro-3-iodo-6-methyl-5-(trifluoromethyl)pyridin-2-amine (Int-BP5)

A solution of 4-chloro-6-methyl-5-(trifluoromethyl)pyridin-2-amine (Int-BP4) (682 mg. 3.24 mmol), iodine (1233 mg. 4.86 mmol), and silyler acetate (811 mg, 4.86 mmol) in DMF (10 mL) was allowed to stir at rt for 18h. The reaction mixture was partitioned between sat. aqueous sodium thiosulfate/EtOAc. The phases were separated and the aqueous phase extracted with EtOAc. The combined organic layers were then washed with 10% aqueous LiCl, dried over magnesium sulfate, and concentrated under reduced pressure. The residue was purified by flash chromatography over silica (0-25% [3:1 EtOAc/EtOH]:hexanes) to afford 4-chloro-3-iodo-6-methyl-5-(trifluoromethyl)pyridin-2-amine. [M+H]⁺+ Found: 337.

### Step 6: 4-chloro-3,6-dimethyl-5-(trifluoromethyl)pyridin-2-amine (Int-BP6)

A solution of 4-chloro-3-iodo-6-methyl-5-(trifluoromethyl)pyridin-2-amine (Int-BP5) (562 mg, 1.670 mmol), methylboronic acid (500 mg, 8.35 mmol), CatacXium A Pd G3 (112 mg, 0.167 mmol), and cesium carbonate (1633 mg, 5.01 mmol) in CPME (4 ml) and Water (1 ml) was allowed to stir at 100° C. for 18h. The reaction mixture was partitioned between EtOAc/water. The phases were separated and the aqueous phase extracted with EtOAc. The combined organic layers were then washed with brine, dried over magnesium sulfate, and concentrated under reduced pressure. The residue was purified by flash chromatography over silica (0-50% EtOAc:hexanes) to afford 4-chloro-3,6-dimethyl-5-(trifluoromethyl)pyridin-2-amine. [M+H]⁺ Found: 225.

### Step 7: 4-chloro-N,N-bis(4-methoxybenyl)-3,6-dimethyl-5-(trifluoromethyl)pyridin-2-amine (Int-BP7)

To a 0° C. cooled solution of 4-chloro-3,6-dimethyl-5-(trifluoromethyl)pyridin-2-amine (Int-BP6) (201 mg, 0.895 mmol) in DMF (9 ml) was added sodium hydride (60% in mineral oil) (107 mg, 2.68 mmol) and the resultant mixture was allowed to stir at 0° C. for 1 h. 4-methoxybenzyl chloride (0.274 ml, 2.013 mmol) was added and the reaction mixture was allowed to stir at 0° C. for 30 minutes. The reaction mixture was partitioned between EtOAc/water. The phases were separated and the aqueous phase extracted with EtOAc. The combined organic layers were then washed with 10% aqueous LiCl, dried over magnesium sulfate, and concentrated under reduced pressure. The residue was purified by flash chromatography over silica (0-10% EtOAc:hexanes) to afford 4-chloro-N,N-bis(4-methoxybenzyl)-3,6-dimethyl-5-(trifluoromethyl)pyridin-2-amine. [M+H]⁺ Found: 465.

### Intermediate BO: 1-bromo-8-fluoro-3-(methoxymethoxy)naphthalene (Int-(BQ2)

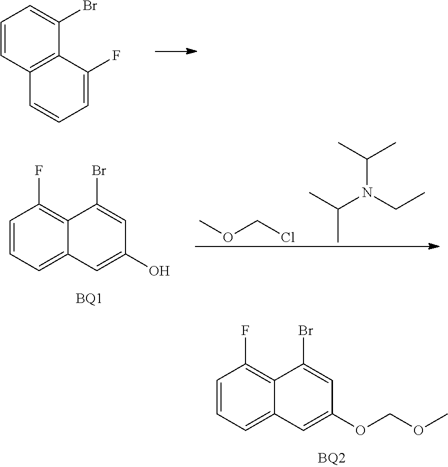

### STEP 1: 4-bromo-5-fluoronaphthalen-2-ol (Int-BQ1)

To a solution of 1-bromo-8-fluoronaphthalene (1.7 g, 7.55 mmol), bis(1,5-cyclooctadiene)dimethoxydiiridium (0.087 g, 0.189 mmol) and 4,4′-di-tert-butyl-2,2′-bipyridine (0.101 g, 0.378 mmol) in Hexane (17 ml) was added 4,4,5,5-tetramethyl-1,3,2-dioxaborolane (1.648 ml, 11.33 mmol) at 25° C. under N₂ atmosphere. The mixture was stirred at 60° C. for 12 h. To the mixture was added NaOH (11.33 ml, 22.66 mmol) and H₂O₂(1.984 ml, 22.66 mmol), then the mixture was stirred at 25° C. for 2h. The reaction was quenched with water (20 mL) and the resulting mixture was extracted with EtOAc (60 mL×3). The combined organic phase was dried over anhydrous Na₂SO₄, filtered and concentrated in vacuo. The residue was purified by flash silica gel chromatography 120 g column, Pet.ether/EtOAc=5/l, 30 min, dry loaded to give the crude product, and it was separated by preparative SFC (Column DAICEL CHIRALPAK AD(250 mm*30 mm, 10 um) Condition Neu-ETOH to give the product 4-bromo-5-fluoronaphthalen-2-ol.

### STEP 2: 1-bromo-8-fluoro-3-(methoxymethoxy)naphthalene (Int-BO2)

To a solution of 4-bromo-5-fluoronaphthalen-2-ol (Int-BQ1) (210 mg, 0.871 mmol) and DIEA (0.456 ml, 2.61 mmol) in anhydrous DCM (2 ml) cooled to 0° C. was slowly added chloro(methoxy)methane (0.165 ml, 2.178 mmol). The solution was warmed to room temperature (20° C.) and stirred for 2 hours. The reaction was quenched with water 5 mL and the resulting mixture was extracted with EtOAc (20 mL×3). The combined organic phase was dried over anhydrous Na₂SO₄, filtered and concentrated in vacuo to give the residue which was purified by flash silica gel chromatography 4 g column, Pet.ether,EtOAc=5/1, dry loaded to give 1-bromo-8-fluoro-3-(methoxymethoxy)naphthalene. H NMR (400 MHz, CHLOROFORM-d) δ=7.58 (d, J=2.4 Hz, 1H), 7.53 (d, J=8.2 Hz, 1H), 7.41-7.34 (m, 2H), 7.09 (ddd, J=0.9, 7.7, 13.1 Hz, 1H), 5.28 (s, 2H), 3.52 (s, 3H).

### Intermediate BR7: 8-bromo-1-chloro-2-fluoro-6-(methoxymethoxy)naphthalene (Int-BR7)

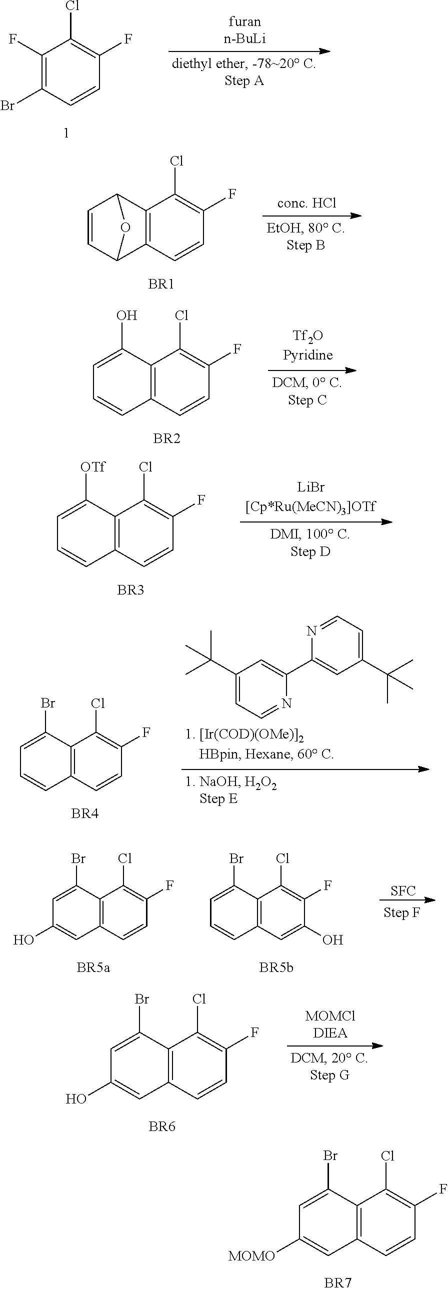

### Step A: 5-chloro-6-fluoro-1,4-dihydro-1,4-epoxynaphthalene (Int-BR1) PGP-.185,C₃

To a solution of I-bromo-3-chloro-2,4-difluorobenzene (15 g, 66.0 mmol) and furan (47g, 693 mmol) in diethyl ether(200 mL) was added butylthium (31.7 mL, 79 mmol. 2.5 M) dropwise at −78° C. The reaction mixture was stirred at 20° C. for 15 h. Reaction was monitored using LCMS, The mixture was quenched with H₂O (200 mL), extracted with EtOAc (150 mL×3), the organic layers were dried with Na₂SO₄, filtered and concentrated. The residue was purified by flash silica gel chromatography column Silica-CS (120 g), Eluent of 3% Ethyl acetate/Petroleum ether gradient to give 5-chloro-6-fluoro-1,4-dihydro-1,4-epoxynaphthalene. MS (ESI) m/z [M+H]⁺ 196.

### Step B: 8-chloro-7-fluoronaphthalen-1-ol (Int-BR2)

A solution of 5-chloro-6-fluoro-1,4-dihydro-1,4-epoxynaphthalene (Int-BR1) (9 g, 45.8 mmol) in EtOH (120 mL) and conc. hydrogen chloride (100 mL, 1.2 mol) was stirred at 80° C. for 15 h. The mixture was concentrated, the residue was purified by flash silica gel chromatography column Silica-CS (80 g), Eluent of 2% Ethyl acetate/Petroleum ether gradient to give 8-chloro-7-fluoronaphthalen-1-ol. ¹H NMR (500 MHz, CHLOROFORM-d) δ 7.92 (s, 1H), 7.75 (dd, J=9.0, 5.5 Hz, 1H), 7.36-7.44 (m, 2H), 7.25-7.32 (m, 1H), 7.09 (d, J=7.5 Hz, 1H).

### Step C: 8-chloro-7-fluoronaphthalen-1-yl trifluoromethanesulfonate (Int-BR3)

To a solution of 8-chloro-7-fluoronaphthalen-1-ol (Int-BR2) (4 g, 20.35 mmol) in DCM (80 mL) was added pyridine (2.414 g, 30.5 mmol) at 0° C. The mixture was stirred at 0° C. for 10 min. Then trifluoromethanesulfonic anhydride (4.11 mL, 24.41 mmol) was added dropwise. The mixture was stirred at 0° C. for 4 h. The mixture was filtered and concentrated, the residue was purified by flash silica gel chromatography column Silica-CS(80 g), Eluent of 1% Ethyl acetate/Petroleum ether gradient to give 8-chloro-7-fluoronaphthalen-1-yl trifluoromethanesulfonate. ¹H NMR (500 MHz, CHLOROFORM-d) δ 7.90 (d, J=8.0 Hz, 1H), 7.85 (dd, J=9.0, 5.5 Hz, 1H), 7.60 (d, J=8.0 Hz, 1H), 7.49-7.55 (m, 1H), 7.45 (t, J=8.5 Hz, 1H). Stem D: 8-bromo-1-chloro-2-fluoronaphthalene (Int-BR4)

To a solution of 8-chloro-7-fluoronaphthalen-1-yl trifluoromethanesulfonate (Int-BR3) (8.5 g, 25.9 mmol) in DMI (110 mL) was added lithium bromide (3.37 g, 38.8 mmol) and tris(acetonitrile)pentamethyl)cyclopentadienylruthenium(ii) trifluoromethanesulfonate (0.658 g, 1.293 mmol). The mixture was stirred at 100° C. for 2 h. The mixture was quenched with H₂O (200 mL), extracted with EtOAc (100 mL×3), the organic layers were washed with H₂O (500 mL×2), dried with Na₂SO₄, filtered and concentrated, the residue was purified by flash silica gel chromatography Silica-CS(80 g), Eluent of 3% Ethyl acetate/Petroleum ether gradient to give 8-bromo-1-chloro-2-fluoronaphthalene. ¹H NMR (500 MHz, CHLOROFORM-d) δ 7.95 (d, J=7.5 Hz, 1H), 7.81 (d, J=8.0 Hz, 1H), 7.77 (dd, J=9.00, 6.0 Hz, 1H), 7.37 (t, J=8.5 Hz, 1H), 7.27-7.30 (m, 1H).

### Step E: 4-bromo-5-chloro-6-fluoronaphthalen-2-ol and 5-bromo-4-chloro-3-fluoronaphthalen-2-ol (Int-BR5a and Int-BR5b)

To a solution of 8-bromo-1-chloro-2-fluoronaphthalene (Int-BR4) (3 g, 11.56 mmol), (1,5-cyclooctadiene)(methoxy)iridium(I)dimer (0.106 g, 0.231 mmol) and 4,4′-di-tert-butyl-2,2′-bipyridine (0.124 g, 0.462 mmol) in hexane (30 mL) was added 4,4,5,5-tetramethyl-1,3,2-dioxaborolane (HBpin) (2.013 mL, 13.87 mmol) at 25° C. under N₂. The mixture was stirred at 60° C. for 15 h. Sodium hydroxide (2 M) (17.34 mL, 34.7 mmol) and hydrogen peroxide (3.57 mL, 34.7 mmol) were added, the mixture was stirred at 20° C. for 2 h. The mixture was quenched with H₂O (20 ml) and adjusted to pH -5 with aqueous HCl (1 M), extracted with EtOAc (15 mL ×3), the organic layers were dried with Na₂SO₄, filtered and concentrated, the residue was purified by flash silica gel chromatography column Silica-CS(40 g), Eluent of 11% Ethyl acetate/Petroleum ether gradient to give the mixture of 4-bromo-5-chloro-6-fluoronaphthalen-2-ol and 5-bromo-4-chloro-3-fluoronaphthalen-2-ol (1:1). 5A+5B: ¹H NMR (500 MHz, CHLOROFORM-d) δ 7.75 (d, J=7.5 Hz, 1H), 7.62 (d, J=8.5 Hz, 1H), 7.59 (d, J=2.0 Hz, 1H), 7.52 (dd, J=9.5, 5.5 Hz, 1H), 7.26-7.32 (m, 1H), 7.23-7.26 (m, 1H), 7.18 (t, J=8.0 Hz, 1H), 7.14 (d, J=2.0 Hz, 1H).

### Step F: 4-bromo-5-chloro-6-fluoronaphthalen-2-ol (Int-BR6)

4-bromo-5-chloro-6-fluoronaphthalen-2-ol (IntBR5b) and 5-bromo-4-chloro-3-fluoronaphthalen-2-ol (Int-BR5b) (1/1) (1.65 g, 2.99 mmol) were separated by SFC (Instrument SFC 5 Method Column DAICEL CHIRALCEL O(250 mm*50 mm, 10 um) Condition 0.1% NH₃H₂O ETOH Begin B 25% End B 25% Gradient Time(min) 100% B Hold Time (min) FlowRate (ml/min) 200 Injections 150) to give 4-bromo-5-chloro-6-fluoronaphthalen-2-ol. ¹H NMR (500 MHz, CHLOROFORM-d) δ 7.74 (d, J=2.5 Hz, 1H), 7.69 (dd, J=9.0, 5.5 Hz, 1H), 7.40 (t, J=8.5 Hz, 1H), 7.27 (d, J=2.5 Hz, 1H).

### Step G: 8-bromo-1-chloro-2-fluoro-64methoxymethoxy)naphthalene (BR7)

To a solution of 4-bromo-5-chloro-6-fluoronaphthalen-2-ol (Int-BR6) (700 mg, 2.54 mmol) and N-ethyl-N-isopropylpropan-2-amine (985 mg, 7.62 mmol) in DCM (10 mL) was added chloro(methoxy)methane (0.287 ml, 3.81 mmol) dropwise. The mixture was stirred at 20° C. for 2 h under N₂. The reaction was quenched with H₂O (15 mL), extracted with DCM (10 mL×2), the organic layers were dried with Na₂SO₄, filtered and concentrated to give 8-bromo-1-chloro-2-fluoro-6-(methoxymethoxy)naphthalene. ¹H NMR (400 MHz, CHLOROFORM-d) δ 7.67 (s, 1H), 7.59 (dd, J=9.2, 5.6 Hz, 1H), 7.34 (d, J=2.4 Hz, 1H), 7.21-7.29 (m, 1H), 5.23 (s, 2H), 3.48 (s, 3H).

### Intermediate BS5: Synthesis of tert-butyl (1S,5R)-3-(6.8-difluoro-2-(3-(R)-3-fluoropyrrolidin-1-yl)-2-hydroxy-2-methylpropoxy,)quinazolin-4-yl)-1-methyl-3,8-diazabicyclo[3.2.1]octane-8-carboxylate (Int-BS5)

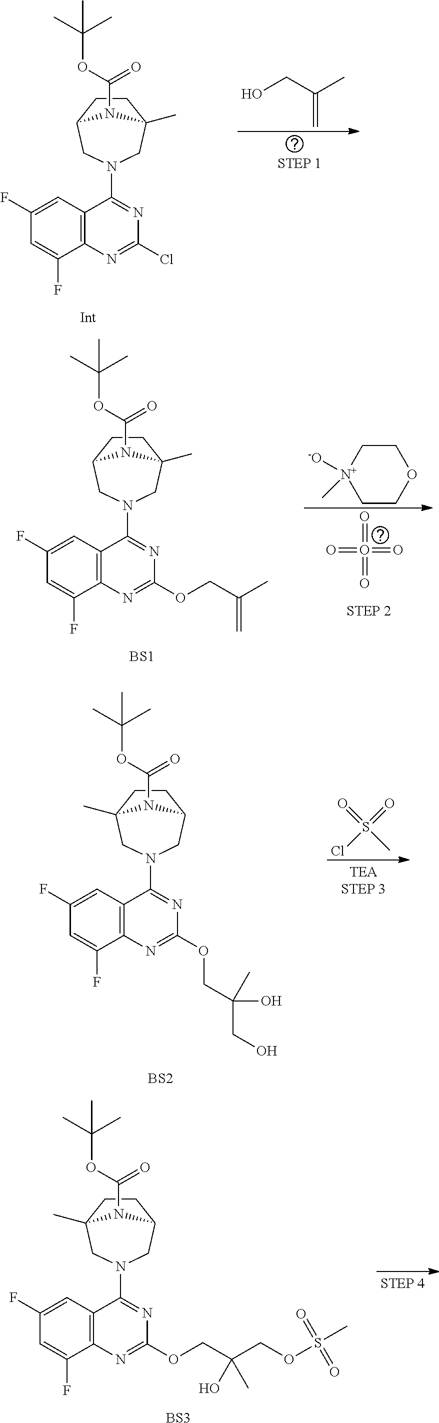

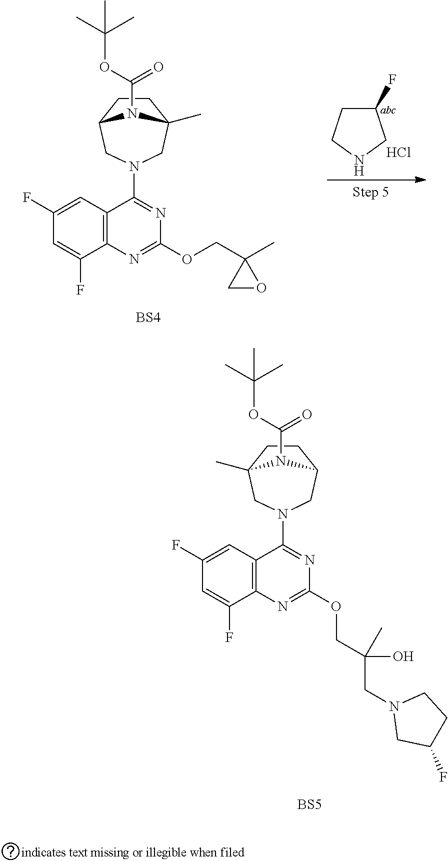

### STEP 1: Tertbutyl (1S,5)-3-(6.8-difluoro-2-((2 methylallyl)oxy)quinazolin-4-yl)1-metyl3,8

To a solution of tert-butyl (1 S,5R)-3-(2-chloro-6,8-difluoroquinazolin-4-yl)-1-methyl-3,8-diazabicyclo[3.2.1]octane-8-carboxylate (Int-D3) (500 mg, 1.177 mmol) in DMSO (5 ml) was added CsF (358 mg, 2.354 mmol), 2-methylprop-2-en-1-ol (127 mg, 1.765 mmol) at 20° C. The mixture was stirred at 100° C. for 12 h and monitored using LC. The mixture was cooled, diluted with water (5 mL), extracted with EtOAc (3×5 mL), dried over Na₂SO₄, filtered and the solvent was evaporated under reduced pressure to give the crude product. The crude product was purified by flash silica gel chromatography 12 g column, Eluent of 0-30% EA/PE gradient to give tert-butyl (1S,5R)-3-(6,8-difluoro-2-((2-methylallyl)oxy)quinazolin-4-yl)-1-methyl-3,8-diazabicyclo[3.2.1]octane-8-carboxylate. MS (ESI) m/z: [M+H]⁺: 461.

### STEP 2: Tert-butyl (1S,5R)-3-(2-(2.2-dihydroxygropoxy)-6,8-difluoroquinazolin-4-yl)-1-methyl-3,8-diazabicyclo[3.2.1]octane-8-carboxylate (Int-BS2)

To a solution of tert-butyl (1S,5R)-3-(6,8-difluoro-2-((2-methylallyl)oxy)quinazolin-4-yl)-1-methyl-3,8-diazabicyclo[3.2.1]octane-8-carboxylate (Int-BS1) (300 mg, 0.651 mmol) in THF (3 ml) and t-BuOH (0.5 ml) was added NMO (114 mg, 0.977 mmol) at 20° C. under N2 atmosphere. The mixture was cooled at −78° C., a solution of osmium(VIII) oxide (16.56 mg, 0.065 mmol) was stirred at −78° C. for 5 min. Then the mixture was warmed at 20° C. for 18 h and monitored using LC. The mixture was diluted with water (3 mL), extracted with EtOAc (3×3 mL), dried over Na₂SO₄, filtered and the solvent was evaporated under reduced pressure to give the crude product. The residue was purified by preparative TLC (SiO₂.ethyl acetate) to give tert-butyl (1S,5R)-3-(2-(2,2-dihydroxypropoxy)-6,8-difluoroquinazolin-4-yl)-1-methyl-3,8-diazabicyclo[3.2.1]octane-8-carboxylate.

### STEP 3: Tert-butyl (1S,5R)-3-(6,8-difluoro-2-(2-hydroxy-2-methyl-3-((methylsulfonyl)oxy)propoxy)quinazolin-4-yl)-1-methyl-3,8-diazabicyclo[3.2.1]octane-8-carboxylate (Int-BS3)

To a solution of tert-butyl (1S,5R)-3-(2-(2,3-dihydroxy-2-methylpropoxy)-6,8-difluoroquinazolin-4-yl)- I-methyl-3,8-diazabicyclo[3.2.1]octane-8-carboxylate (Int-BS2) (290 mg, 0.586 mmol) in DCM (3 ml) was added TEA (0.245 ml, 1.759 mmol) and MsCI (0.143 ml. 1.833 mmol) at 20° C. under N₂ atmosphere. The mixture was stirred at 20° C. for 1 hr and monitored by LC. The mixture was diluted with water (3 mL), extracted with DCM (3×3 mL), dried over Na₂SO₄, filtered and the organic was evaporated under reduced pressure to give the crude tert-butyl (1S,5R)-3-(6,8-difluoro-2-(2-hydroxy-2-methyl-3-((methylsulfonyl)oxy)propoxy)quinazolin-4-yl)-1-methyl-3,8-diazabicyclo[3.2.1]octane-8-carboxylate as brown oil, which was used directly in the next step without further purification. MS (ESI) m/z: calc'd [M+H]⁺: 573.

### STEP 4: Tert-butyl (1S,5R)-3-(6.8-difluoro-2-((2-methyloxiran-2-yl)methoxy)quinazolin-4-yl)-1-methyl-3,8-diazabicyclo[3.2.1]octane-8-carboxylate (Int-BS4)

To a solution of tert-butyl (1S,5R)-3-(6.8-difluoro-2-(2-hydroxy-2-methyl-3-((methylsulfonyl)oxy)propoxy)quinazolin-4-yl)-1-methyl-3,8-diazabicyclo[3.2.1]octane-8-carboxylate (Int-BS3) (250 mg, 0.437 mmol) in MeOH (3 ml) was added K₂CO₃ (181 mg, 1.310 mmol) at 20° C. under N₂ atmosphere. The mixture was stirred at 50° C. for 2 h. The mixture was cooled, diluted with water (3 mL), extracted with EtOAc (3×3 mL), dried over Na₂SO₄, filtered and the solvent was evaporated under reduced pressure to give the crude product. The residue was purified by preparative TLC (SiO₂, petroleum ether:ethyl acetate=3: 1) to give tert-butyl (1S,5R)-3-(6,8-difluoro-2-((2-methyloxiran-2-yl)methoxy)quinazolin-4-yl)-1-methyl-3,8-diazabicyclo[3.2.1]octane-8-carboxylate. MS (ESI) m/z: calc'd [M+H]⁺: 477.

### STEP 5: Tert-butyl (1S,5R)-3-(6.8-difluoro-2-(3-((R)-3-fluoropyrrolidin-1-yl)-2-hydroxy-2-methylpropoxy)quinazolin-4-yl)-1-methyl-3,8-diazabicyclo[3.2.1]octane-8-carboxylate (Int-BS5)

To a solution of tert-butyl (1 S,5R)-3-(6,8-difluoro-2-((2-methyloxiran-2-yl)methoxy)quinazolin-4-yl)- I-methyl-3,8-diazabicyclo[3.2.1]octane-8-carboxylate (Int-BS4) (170 mg, 0.357 mmol) in THF (2 ml) was added K₂CO₃ (247 mg, 1.784 mmol) and (R)-3-fluoropyrrolidinehydrochloride (224 mg, 1.784 mmol) at 20° C. under N₂ atmosphere. The mixture was stirred at 80° C. for 18 h and monitored using LC. The mixture was cooled, diluted with water (3 mL), extracted with EtOAc (3×3 mL), dried over Na₂SO₄, filtered and the solvent was evaporated under reduced pressure to give the crude product. The residue was purified by preparative TLC (SiO₂, petroleum ether:ethyl acetate=1:1) to give tert-butyl (1S,5R)-3-(6,8-difluoro-2-(3-((R)-3-fluoropyrrolidin-1-yl)-2-hydroxy-2-methylpropoxy)quinazolin-4-yl)-1-methyl-3,8-diazabicyclo[3.2.1]octane-8-carboxylate. MS (ESI) m/z: calc'd [M+H]⁺: 566.

### Intermediate BT7: Benzyl (1S)-3-(2-chloro-5.6.8-trifluoroquinazolin-4-yl)-1-methyl-38-diazabicyclo [3.2.1]octane-8-carboxylate (Int-BT7)

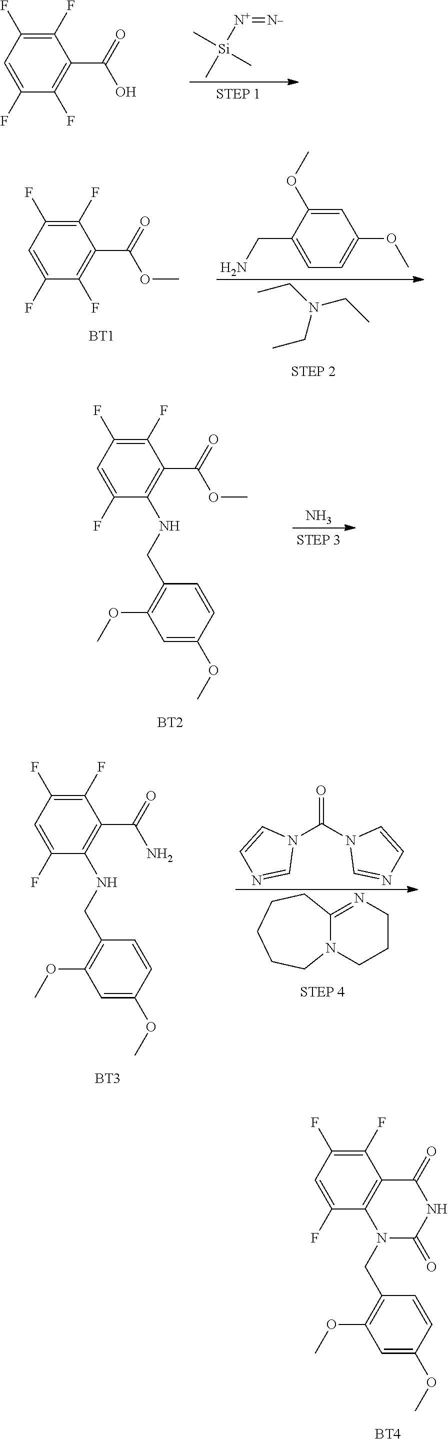

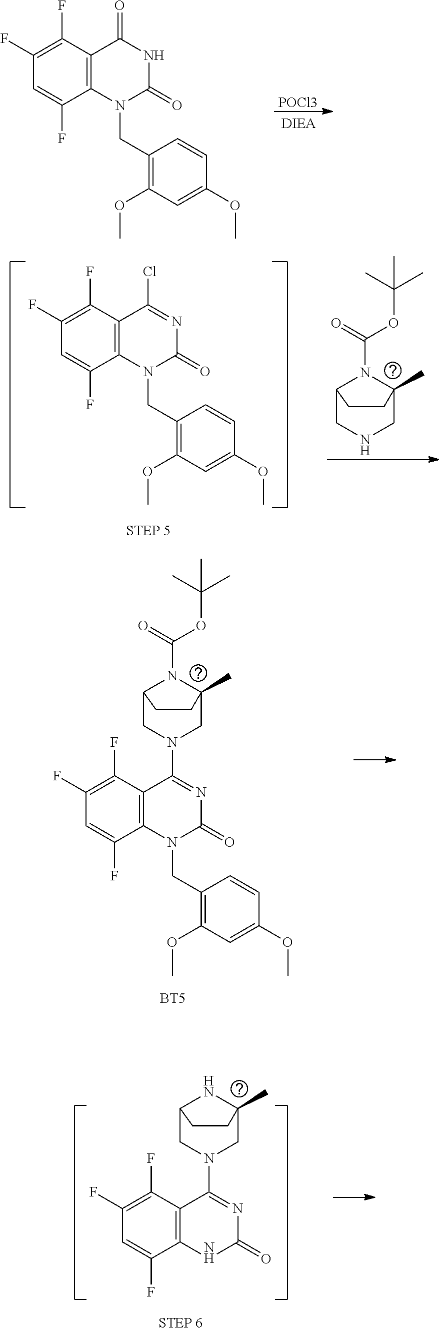

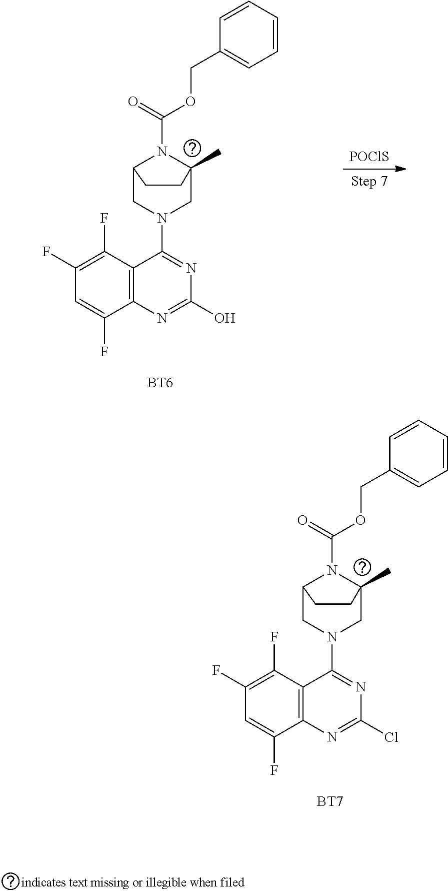

### STEP 1: Methyl 2,3,5,6-tetrafluorobenzoate (Int-BT1)

A 50 ml flask was charged with 2,3,5,6-tertrafluorobenzoic acid (2.005 g, 10.33 mmol), Toluene (10 ml) and Methanol (1.000 ml). The mixture was stirred at rt while TMS-Diazotnethane (2 M in diethyl ether) (6.20 ml, 12.40 mmol) was added dropwise over 10 minutes. After addition, the mixture was stirred at rt for 16 h. The mixture was treated dropwise at frtwith acetic acid (0.591 ml, 10.33 mmol) and then concentrated to afford a dark brown-colored residue. The residue was dissolved in minimal dichloromethane and applied to a 80 g silica gel column. Purification by column chromatography on silica gel (80 g, 0 to 50% EtOAc/hexanes was carried out to afford Methyl 2,3,5,6-tetrafluorobenzoate. MS (ESI): m/z (M+H)⁺ 654.

### STEP 2. Methyl 2-((2.4-dimethoxybenzyl)amino)-3,5,6-trifluorobenzoate (Int-BT2

A 40 ml scintillation vial was charged at rt with methyl 2,3,5,6-tetrafluorobenzoate (Int-BT1) (500 mg, 2.403 mmol), 2.4-dimethoxybenzylamine (803 mg, 4.81 mmol), Toluene (10 ml) and triethylamine (1.005 ml, 7.21 mmol). The vial was capped and heated at 110° C. for 4 hours. A precipitate formed over the first hour of heating. The reaction was cooled to rt and diluted with ethyl acetate. The organic layer was washed with water and brine, dried with anhydrous sodium sulfate, filtered and the filtrate was concentrated. The residue was purified by column chromatography on silica gel −80g; 0 to 50% EtOAc/hexanes to give Methyl 2-((2,4-dimethoxybenzyl)amino)-3,5,6-trifluorobenzoate. MS (ESI): m/z (M+H)⁺ Na 378.

### STEP 3: 2-((2.4-dimethoxybenzyl)amino)-3,5,6-trifluorobenzamide (Int-BT3)

Methyl 2-((2,4-dimethoxybenzyl)amino)-3,5,6-trifluorobenzoate (Int-BT2) (630 mg, 1.773 mmol) in ammonia (7 M in methanol) (5 ml, 231 mmol) was added to a large crimp top microwave vial and heated at 80° C. for 24 h. The reaction was cooled to rt and concentrated to afford 2-((2,4-dimethoxybenzyl)amino)-3,5,6-trifluorobenzamide. This was used in the next step without further purification. MS (ESI): m/z (M+H)⁺ Na 363.

### STEP 4: 1-(2.4-dimethoxybenzyl)-5.6.8-trifluoroquinazoline-2,4(1H,3H)-dione (Int-BT4)

2-((2,4-dimethoxybenzyl)amino)-3,5,6-trifluorobenzamide (Int-BT3) (603 mg, 1.773 mmol) in Dichloromethane (5.0 ml) was treated at rt with CDI (431 mg, 2.66 mmol) and DBU (0.401 ml, 2.66 mmol). The mixture was stirred at 35° C. for 1 h. The mixture was directly applied to a 40g silica gel column and purified by column chromatography on silica gel (0 to 10% MeOH/dichloromethane). LCMS indicates a mixture of 1-(2,4-dimethoxybenzyl)-5,6,8-trifluoroquinazoline-2,4(1H,3H)-dione and methyl ester of the starting material that was carried through. This solid was triturated in dichloromethane and solids collected by vacuum filtration to afford 1-(2,4-dimethoxy benzyl)-5,6,8-trifluoroquinazoline-2,4(1H,3H)-dione. MS (ESI): m/(M+H)⁺ 389.

### STEP 5: Tert-butyl (1S)-3-(1-(2.4-dimethoxybenzyl)-5,6,8-trifluoro-2-oxo-1,2-dihydroquinazolin-4-yl)-1-methyl-3,8-diazabicyclo[3.2.1]octane-8-carboxylate (Int-BT5)

Step 1: 1-(2,4-dimethoxybenzyl)-5,6,8-trifluoroquinazoline-2,4(1H,3H)-dione (Int-BT4) (260 mg, 0.710 mmol) in Acetonitrile (4.0 ml) was treated with DIEA (0.4% ml, 2.84 mmol) and POCl₃ (0.132 ml, 1.420 mmol). The reaction was heated at 75° C. for 1 h. The reaction was cooled to rt and concentrated under reduced pressure and azeotroped with acetonitrile 4 ml.

Step 2: The above residue was dissolved in Acetonitrile (4.00 ml) and treated with DIEA (0.496 ml, 2.84 mmol) and tert-butyl (1S)-1-methyl-3,8-diazabicyclo[3.2.1]octane-8-carboxylate (321 mg, 1.420 mmol) and the reaction was stirred at rt for 2 h. This was concentrated and the residue was loaded with dichloromethane onto a 40g silica gel column and purified by column chromatography on silica gel (0 to 100% 1[1:3] EtOH/EtOAc]/hexanes to afford tert-butyl (1S)-3-(1-(2,4-dimethoxybenzyl)-5,6,8-trifluoro-2-oxo-1,2-dihydroquinazolin-4-yl)-1-methyl-3,8-diazabicyclo[3.2.1]octane-8-carboxylate which was used as is without further purification. MS (ESI): m/z (M+H)⁺ 575.

### STEP 6. Benzyl (1S)-1-methyl-3-(5,6,8-trifluoro-2-hydroxyquinazolin-4-yl)-3,8-diazabicyclo[3.2.1]octane-8-carboxylate (Int-BT6)

Tert-butyl (1S)-3-(1-(2,4-dimethoxybenzyl)-5,6,8-trifluoro-2-oxo-1,2-dihydroquinazolin-4-yl)-1-methyl-3,8-diazabicyclo[3.2.1]octane-8-carboxylate (Int-BT5) (270 mg, 0.470 mmol) was stirred in TFA (10 ml) at rt for 18 h. Reaction was monitored by LC indicating intermediate. The reaction mixture was concentrated in vacuo and the residue dissolved in dichloromethane (20 ml) and concentrated and then dried for 30 minutes to afford the intermediate as a purple gum. The intermediate was dissolved in Dichloromethane (10.00 ml) and treated with DIEA (0.410 ml, 2.349 mmol), cooled to 0° C. and treated with Cbz-CI (0.080 ml, 0.564 mmol). The reaction was stirred at 0° C. for 15 minutes and then the ice bath was removed and the mixture was stirred at rt for 15 minutes. Reaction mixture was directly dry loaded onto a 40g silica gel column and purified by column chromatography on silica gel (40g, 0 to 100% [1:3 EtOH/EtOAc]/hexanes to afford benzyl (1S)-1-methyl-3-(5,6,8-trifluoro-2-hydroxyquinazolin-4-yl)-3,8-diazabicyclo[3.2.1]octane-8-carboxylate. MS (EST): m/z (M+H)⁺ 459.

### STEP 7: Benzyl (1S)-3-(2-chloro-5.6.8-trifluoroguinazolin-4-yl)-1-methyl-3,8-diazabicyclo [3,2,1]octane-8-carboxylate (Int-BT7)

Benzyl (1S)-1-methyl-3-(5,6,8-trifluoro-2-hydroxyquinazolin-4-yl)-3,8-diazabicyclo[3.2.1]octane-8-carboxylate (Int-BT6) (270 mg, 0.589 mmol) in POCl₃ (2000 μl, 21.46 mmol) was heated at 65° C. for 30 minutes. The reaction was cooled to rt and concentrated. The resulting amber-coloured syrup was dissolved in dichloromethane (2 ml) and treated with tmethylamine (0.5 ml). The mixture was loaded onto a dry 40g silica gel column and purified by column chromatography on silica gel using 0 to 100% [1:3 EtOH/EtOAc]/hexanes to afford benzyl (1S)-3-(2-chloro-5,6,8-trifluoroquinazolin-4-yl)-1-methyl-3,8-diazabicyclo [3.2.1]octane-8-carboxylate. This was used without further purification. MS (ESI): m/z (M+H)⁺ 477.

### Intermediate BU7: 4-(tert-butoxy)-6-chloro-8-fluoro-2-(methylthio)quinazoline (Int-BU7)

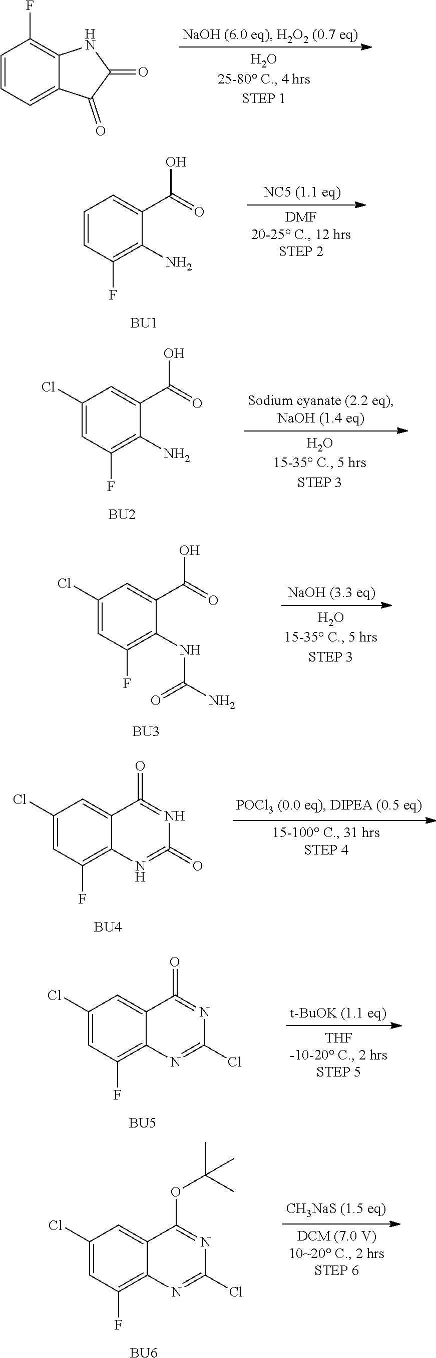

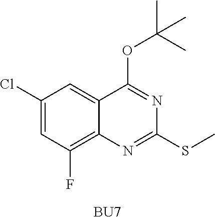

### STEP 1: 2-amino-3-fluorobenzoic acid (Int-BUl)

To a reactor was added H₂O (32 L) at 25° C. 7-fluoroindoline-2,3-dione (3.2 kg, 19 mol) and NaOH (4.6 kg, 116 mol) were added into the reactor at rt. The mixture was stirred at 75-80° C. for 1 hr. H₂O₂(1.5 kg, 13 mol. 30% purity) was added to the reaction slowly dropwise at 75-80° C. over a period of 2 hrs. The mixture was stirred at 75-80° C. for 1 hr. The reaction was monitored by TLC. Na₂SO₃ (10 kg) and H₂O (80 L) were added to another reactor separately and this solution was added to the reaction mixture slowly at 0-10° C. within 2 hrs. 1 M HCl (120 L) was added into the reaction mixture. The reaction was filtered and the filter cake was washed with water (10 L). The filter cake was dried to give 2-amino-3-fluorobenzoic acid.

### STEP 2: 2-amino-5-chloro-3-fluorobenzoic acid (Int-BU2)

To DMF (14 L) at 20-25° C. was added 2-amino-3-fluorobenzoic acid (Int-BUl) (2.0 kg, 12 mol). NCS (1.8 kg, 14 mol) was added and the reaction mixture was stirred at rt for 12 hrs. The reaction was monitored using HPLC. Slowly the reaction mixture was transferred to another flask with H₂O (28 L) at 0˜10° PC over 2 hrs. The reaction mixture was filtered and the filter cake was washed with H₂O (10 L) and then dried to give 2-amino-5-chloro-3-fluorobenzoic acid. ¹H NMR: (400 MHz, DMSO-d₆) δ: 7.41-7.47 (m, 1H), 7.50-7.53 (m, 1H).

### STEP 3: 6-chloro-8-fluoroquinazoline-2,4(1H,3H)-dione (Int-BU4)

Step 1: To H₂O (24 L) at 25° C., 2-amino-5-chloro-3-fluorobenzoic acid (Int-BU2) (2.4 kg, 12 mol) was added. NaOH (0.7 kg, 17 mol) and NaCNO (1.8 kg, 27 mol) were added to the reaction and the mixture was stirred between 15-35° C. for 1 hr. 1 M HCl was added to adjust the pH between 5.8-6,3 and the reaction was stirred for 4 hours between 15-35° C. to give 5-chloro-3-fluoro-2-ureidobenzoic acid (Int-BU3).

Step 2: NaOH (1.6 kg, 41 mol) was added to the solution of 5-chloro-3-fluoro-2-ureidobenzoic acid (Int-BU3) and the reaction mixture was stirred for 2 hours between 15-35° C. The reaction mixture was filtered and the filter cake was washed with H₂O (10 L). The filter cake was re-suspended in H₂O (24 L) at 15-35° C. and 1 M HCl was added to adjust the pH between 1-2. The mixture was stirred for an hour and then filtered. The filter cake was washed with H₂O (10 L) and resuspended in acetone (12 L) at 15-35° C. and then filtered. The filter cake was again washed with MeOH (5.0 L), dried under vacuum between 40-45° C. for 12 hrs to give 6-chloro-8-fluoroquinazoline-2,4(1H,3H)-dione. ¹H NMR: (400 MHz, DMSO-d₆) d: 7.63-7.69 (m, 1H), 7.77-7.85 (m, 1H).

### STEP 4: 2,4,6-trichloro-8-fluoroquinazoline (Int-BU5)

To POCl₃ (11 kg, 77 mol), 6-chloro-8-fluoroquinazoline-2,4(1H,3H)-dione (Int-BU4) (1.8 kg, 8.6 mol) was added at 25° C. DIPEA (0.5 kg, 4.1 mmol) was added and reaction mixture was stirred for 30 mins. The reaction mixture was warmed to 100-110° C. and stirred for 30 hrs. The reaction was monitored via HPLC. After completion, the reaction was concentrated under reduced pressure to about 2.0 L. H₂O (50 L) was added and slowly the reaction mixture was transferred to another reactor at 10-30° C. over 2 hrs and then stirred for 30 mins. The aqueous solution was extracted with the MTBE (10 L×3). The organic layers were combined and washed with brine (10 L×2), dried over Na₂SO₄, filtered and the filtrate concentrated under reduced pressure to give a residue. This residue was vacuum dried to give 2,4,6-trichloro-8-fluoroquinazoline. ¹H NMR: (400 MHz, CDCl₃) d: 7.57-7.65 (m, 1H), 7.98 (s, 1H).

### STEP 5: 44tert-butoxy)-2,6-dichloro-8-fluoroquinazoline (Int-BU6)

To THF (15 L) 2,4,6-trichloro-8-fluoroquinazoline (Int-BU5) (1.5 kg, 5.9 mol) was added at 20° C. t-BuOK (0.7 kg, 6.5 mol, 1 M in THF) was added in batches to the reaction between -10-5° C. over 30 mins and the reaction was stirred for another 30 mins. The reaction was warmed to 15-20° C. and stirred at this temperature for 2 hrs. 0.5 M HCl (2.0 L) was added to the reaction and then tis was transferred to another reactor at 0-10° C. over 10 mins. H₂O (15 L) was added and the aq. solution was extracted with MTBE (5.0 L×3). The organic layers were combined and washed with brine (5.0 L), dried over Na₂SO₄, filtered and the filtrate obtained was concentrated under reduced pressure to give a residue. This was re-crystallized from MeOH (5.0 L) at 25° C. to give 4-(tert-butoxy)-2,6-dichloro-8-fluoroquinazoline. ¹H NMR: (400 MHz, CDCl₃) δ: 1.68 (s, 9H), 7.39-7.47 (m, 1H), 7.72-7.79 (m, 1H).

### STEP 6: 4-(tert-butoxy)-6-chloro-8-fluoro-2-(methylthio)quinazoline (Int-BU7)

To DCM (3.5 L), 4-(tert-butoxy)-2,6-dichloro-8-fluoroquinazoline (Int-BU6) (5(0) g, 1.7 mol) was added at rt. CH₃NaS (179 g, 2.6 mol) was added slowly to the reaction at 10-20° C. in portions over 30 mins and stirred for 2 hrs. H₂O (7.0 L) was added and slowly the reaction was transferred to another reactor at 0-10° C. over 10 mins. The reaction was then filtered and the filter bed was washed with MeOH (5.0 L). The crude residue obtained was purified by reverse phase HPLC (0.1% FA condition) to give 4-(tert-butoxy)-6-chloro-8-fluoro-2-(methylthio)quinazoline. ¹H NMR: (400 MHz, CDCl₃) δ: 1.65 (s, 9H), 2.54-2.59 (m, 3H), 7.29-7.37 (m, 1H), 7.62-7.71 (m, 1H).

### Intermediate BV4: Tert-butyl (4-bromo-5-chloro-1-fluoronaphthalen-2-yl)(tert-butoxycarbonyl)carbamate (Int-BV4)

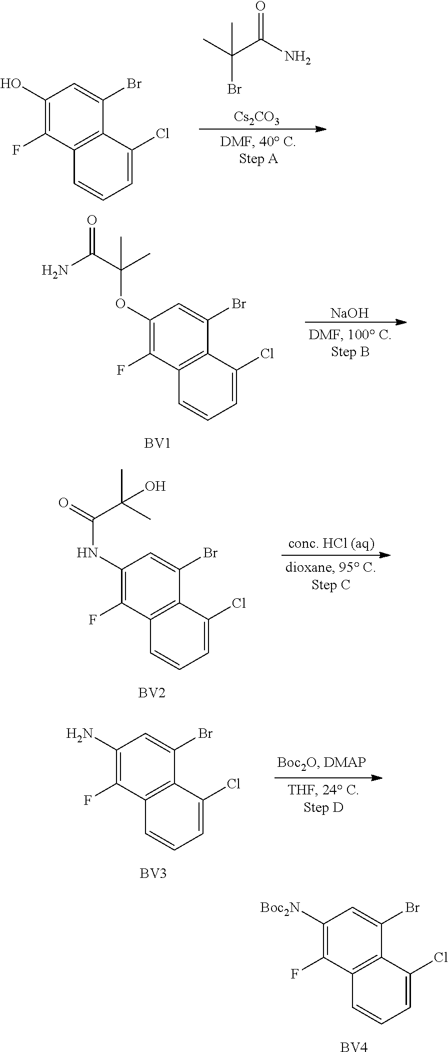

### Step A: 2-((4-bromo-5-chloro-1-fluoronaahthalen-2-yl)oxy)-2-methylpropanamide (Int-BV1)

2-Bromo-5-chloro-1-fluoronaphthalen-2-ol (442 mg, 1.604 mmol) was added into a 30 mL vial. The reaction vessel was evacuated and backfilled with a balloon of nitrogen three times. DMF (5.3 mL) was added into the reaction vessel to dissolve the starting material. Cesium carbonate (1.73 g, 5.29 mmol) and 2-bromo-2-methylpropanamide (799 mg, 4.81 mmol) were added into the reaction vessel. The resulting suspension was heated at 40° C. for 16h. The product mixture was diluted with ether (100 mL) and washed three times with saturated sodium chloride aqueous solution. The organic layer was dried over sodium sulfate. The dried solution was filtered and the filtrate was concentrated to dryness. The residue obtained was used directly into the next step without purification.

### Step B: N44-bromo-5-chloro-1-fluoronaphthalen-2-yl)-2-hydroxy-2-methylpropanamide (Int-BV2)

2-((4-Bromo-5-chloro-1-fluoronaphthalen-2-yl)oxy)-2-methylpropanamide (Int-BV1) (578 mg, 1.60 mmol) and sodium hydroxide (513 mg, 12.8 mmol) were heated in DMF (4.0 mL) for 1 h at 100° C. The product mixture was cooled down to room temperature and diluted with ether (100 mL). The diluted product mixture was washed three times with saturated sodium bicarbonate aqueous solution (3-20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 66% ethyl acetate-hexanes, linear gradient to afford N-(4-bromo-5-chloro-1-fluoronaphthalen-2-yl)-2-hydroxy-2-methylpropanamide. ¹H NMR (500 MHz, Acetonitrile-di) δ 9.21 (s, 1H), 8.88 (d, J=7.7 Hz, 1H), 8.13 (d, J=8.5 Hz, 1H), 7.75 (d, J=7.5 Hz, 1H), 7.56 (t, J=8.0 Hz, 1H), 4.04 (s, 1H), 1.50 (s, 6H).

### Step C: 4-bromo-5-chloro-1-fluoronaphthalen-2-amine (Int-BV3)

N-(4-Bromo-5-chloro-1-fluoronaphthalen-2-yl)-2-hydroxy-2-methylpropanamide (Int-BV2) (491 mg, 1.36 mmol) was heated in dioxane (2,3 mL) and conc. HCl (2,3 mL) for 1 h at 95° C. The product mixture was basified with saturated sodium carbonate aqueous solution. The basified product mixture was extracted three times with DCM (3×30 mL). The organic layer was dried over sodium sulfate. The dried solution was filtered and the filtrate was concentrated to dryness. The residue obtained was purified by flash column chromatography with 40-g silica gel column, eluting with hexanes initially, grading to 100% ethyl acetate, linear gradient to afford 4-bromo-5-chloro-1-fluoronaphthalen-2-amine. ¹H NMR (500 MHz, Chloroform-d) 7.91 (dt, J=8.4, 1.3 Hz, 1H), 7.52 (d, J=8.6 Hz, 1H), 7.46 (d, J=7.4 Hz, 1H), 7.34 (t, J=7.9 Hz, 1H), 3.94 (s, 2H).

### Step D: Tert-butyl (4-bromo-5-chloro-1-fluoronaphthalen-2-yl(tert-butoxycarbonylkarbamate (Int-BV4)

2-Bromo-5-chloro-1-fluoronaphthalen-2-amine (Int-BV3) (350 mg, 1.28 mmol) was dissolved in THF (4.3 mL). N,N-Dimethylpyridin-4-amine (312 mg, 2.55 mmol) and di-tert-butyl dicarbonate (306 mg, 1.40 mmol) were added into the reaction vessel. The resulting mixture was stirred for 3d at 24° C. The product mixture was diluted with 1:1 v/v ether:hexanes (50 mL). The diluted product mixture was washed three times with saturated sodium bicarbonate aqueous solution (3-20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford the desired product tert-butyl (4-bromo-5-chloro-1-fluoronaphthalen-2-yl)(tert-butoxycarbonyl)carbamate. ¹H NMR (500 MHz, Acetonitrile-d₃) δ 8.21 (d, J=8.4 Hz, 1H), 7.95 (d, J=7.7 Hz, 1H), 7.87 (d, J=7.5 Hz, 1H), 7.62 (t, J=8.0 Hz, 1H), 1.43 (d, J=1.2 Hz, 18H).

### Intermediate BX4: Tert-butyl (1S,5R,6S)-6-methoxy-1.5-dimethyl-3,8-diazabicyclo[3,2,1]octane-8-carboxylate (Int-BX4)

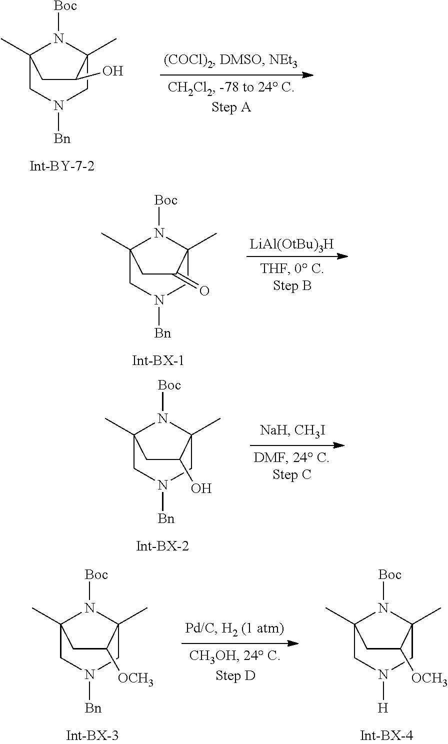

### Step A: tert-butyl (1S,5R)-3-benzyl-1.5-dimethyl-6-oxo-3,8-diazabicyclo[3.2.1]octane-8-carboxylate (Int-BX-1. Peak 2)

DMSO (169 mg, 2.16 mmol) was added in a 30 mL vial. The reaction vessel was evacuated and backfilled with a balloon of nitrogen three times. DCM (7.2 mL) was added into the reaction vessel. The resulting mixture was cooled down to −78° C. A 2M DCM solution of oxalyl dichloride (149 μL, 1.73 mmol) was added dropwise into the reaction vessel. The resulting mixture was stirred for 15 min. A solution of tert-butyl (1S,5R,6R)-3-benzyl-6-hydroxy-1,5-dimethyl-3,8-diazabicyclo[3.2.1]octane-8-carboxylate (Int-BY7-2) (500 mg, 1.44 mmol) and DCM (7.2 μL) were added into the reaction vessel dropwise. The resulting mixture was stirred for 15 min. Then triethylamine (1.00 mL, 7.22 mmol) was added dropwise into the reaction vessel. The resulting mixture was stirred for 15 min at −78° C. and then warmed up to room temperature. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 40% ethyl acetate-hexanes, linear gradient to afford tert-butyl (1S,5R)-3-benzyl-1,5-dimethyl-6-oxo-3,8-diazabicyclo[3.2.1]octane-8-carboxylate. MS (EST): m/z (M+H)⁺ 345.

### Step B: Tert-butyl (1S,5R,6S)-3-benzyl-6-hydroxy-1,5-dimethyl-3,8-diazabicyclo[3.2.1]octane-8-carboxylate (Int-BX-2)

Tert-Butyl(1S,5R)-3-benzyl-1,5-dimethyl-6-oxo-3,8-diazabicyclo[3.2.1]octane-8-carboxylate (Int-BX-1) (438.4 mg, 1.273 mmol) was added in a 30 mL vial. The reaction vessel was evacuated and backfilled with a balloon of nitrogen three times. THF (6.4 mL) was added into the reaction vessel. The resulting mixture was cooled down to 0° C. Lithium tri-tert-butoxyaluminum hydride (2.55 μL, 2.55 mmol, 1M solution in THF) was added dropwise into the reaction mixture. The resulting mixture was stirred for 30 min at 0° C. and then quenched with saturated Rochelle's salt solution (1 mL). The resulting mixture was stirred for 15 min and the resulting mixture was extracted three times with DCM (3×30 mL). The organic layer was dried over sodium sulfate. The dried solution was filtered and the filtrate was concentrated to dryness. The residue obtained was purified by flash column chromatography with 40-g silica gel column, eluting with hexanes initially, grading to 40% ethyl acetate-hexanes, linear gradient to afford tert-butyl (1S,5R,6S)-3-benzyl-6-hydroxy-1,5-dimethyl-3,8-diazabicyclo[3.2.1]octane-8-carboxylate. MS (ESI): m/z (M+H)⁺ 347.

### Step C: Tert-butyl (1S,5R,6S-3-benzyl-6-methoxy-1.5-dimethyl-3,8-diazabicyclo[3.2.1]octane-8-carboxylate (Int-BX-3)

Tert-Butyl (1S,5R,6S)-3-benzyl-6-hydrow-1,5-dimethyl-3,8-diazabicyclo[3.2.1]octane-8-carboxylate (Int-BX-2) (425.2 mg, 1.227 mmol) was added into a 30 mL vial. The reaction vessel was evacuated and backfilled with a balloon of nitrogen three times. DMF (6.1 mL) was added into the reaction vessel. Sodium hydride (98 mg, 2.5 mmol, 60 wt % suspension in mineral oil) was added into the reaction vessel. The resulting mixture was stirred for 30 min at 24° C. Then iodomethane (153 μL, 2.45 mmol) was added into the reaction vessel. The resulting mixture was stirred for 16h at 24° C. The product mixture was diluted with ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 90% ethyl acetate-hexanes, linear gradient to afford tert-butyl (1S,5R,6S)-3-benzyl-6-methoxy-1,5-dimethyl-3,8-diazabicyclo[3.2.1]octane-8-carboxylate.

### Step D: Tert-butyl (1S,5R,6S)-6-methoxy-1,5-dimethyl-3,8-diazabicyclo[3.2.1]octane-8-carboxylate (Int-BX-4)

Tert-Butyl (1S,5R,6S)-3-benzyl-6-methoxy-1,5-dimethyl-3,8-diazabicyclo[3.2.1]octane-8-carboxylate (Int-BX-3) (50 mg, 0.14 mmol) and palladium on activated charcoal (7.4 mg, 6.9 μmol) were added in a 30 mL vial. The reaction vessel was evacuated and backfilled with a balloon of nitrogen three times. Methanol (1.4 mL) was added into the reaction vessel. The reaction vessel was evacuated and backfilled with a balloon of nitrogen three times. The reaction mixture was stirred for 4h. The product mixture was thoroughly purged with nitrogen and filtered. The filtrate was concentrated to dryness. The residue containing tert-butyl (1S,5R,6S)-6-methoxy-1,5-dimethyl-3,8-diazabicyclo[3.2.1]octane-8-carboxylate obtained this way was used directly in the next step without purification.

### Intermediate BY10: Tert-butyl (1R,5S,6S)-6-(methoxymethoxy)-1,5-dimethyl -3,8-diazabicyclo[3.2.1]octane-8-carboxylate (Int-BY9-1)

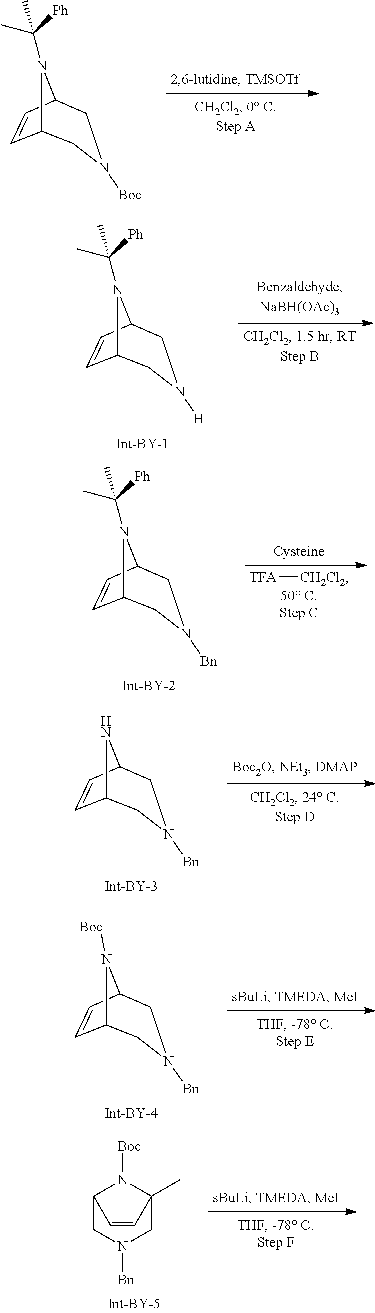

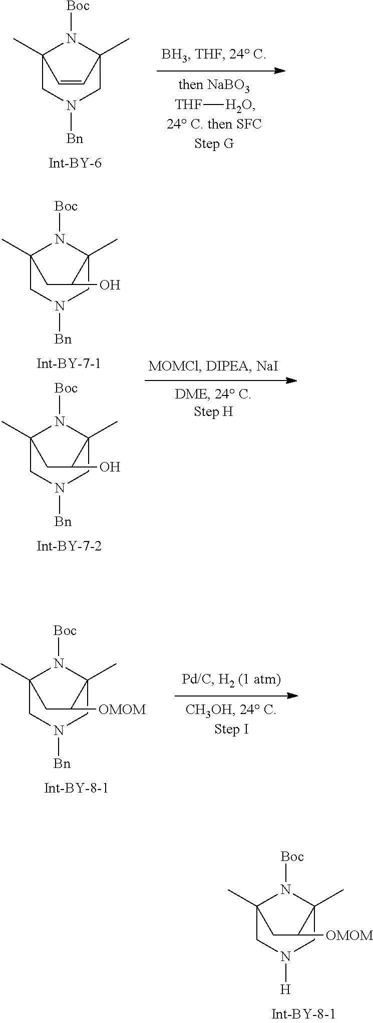

### Step A: (1R,5S)-8-(2-phenyloropan-2-yl)-3,8-diazabicyclo[3.2.1]oct-6-ene (Int-BY1)

Tert-Butyl (1R,5S)-8-(2-phenylpropan-2-yl)-3,8-diazabicyclo[3.2.1]oct-6-ene-3-carboxylate (12.0 g, 36.5 mmol) was added in a 250 mL round bottomed flask. The reaction vessel was evacuated and backfilled with a balloon of nitrogen three times. DCM (91 mL) and 2,6-dimethylpyridine (12.8 mL, 110 mmol) were added into the reaction vessel. The resulting mixture was cooled down to 0° C. Trimethylsilyl trifluoromethanesulfonate (13.2 mL, 73.1 mmol) was added dropwise into the reaction vessel. The resulting mixture was stirred for 1 h at 0° C. The product mixture was quenched by carefully adding saturated sodium bicarbonate aqueous solution (15 mL) dropwise. Vigorous gas evolution was observed within the first 0.5 mL addition of the solution. The quenched product mixture was extracted three times with dichloromethane (3×100 mL). The organic layers were dried and the dried solution was filtered. The filtrate was concentrated to dryness. The residue obtained containing (1R,5S)-8-(2-phenylpropan-2-yl)-3,8-diazabicyclo[3.2.1]oct-6-ene was used directly in the next step without purification.

### Step B: (1R,5S)-3-benzyl-8-(2-phenyloropan-2-yl)-3,8-diazabicyclo[3.2.1]oct-6-ene (Int-BY2)

(1R,5S)-8-(2-Phenylpropan-2-yl)-3,8-diazabicyclo[3.2.1]oct-6-ene (Int-BY1) (nominally 36.5 mmol) was dissolved in DCE (91 mL). Benzaldehyde (4.08 mL, 40.2 mmol) was added into the reaction vessel and the resulting mixture was stirred until all the starting material was dissolved. Sodium triacetoxyhydroborate (12.4 g, 58.4 mmol) was added into the reaction vessel in one portion. The resulting mixture was stirred for 1.5 h at 24° C. The product mixture was quenched with saturated sodium bicarbonate aqueous solution (3 mL). The quenched product mixture was poured into a separatory funnel charged with saturated sodium carbonate aqueous solution (50 mL) and DCM (50 mL). The aqueous layer was extracted three times with DCM (3×100 mL). The organic layer was dried over sodium sulfate. The dried solution was filtered and the filtrate was concentrated to dryness. The residue obtained was purified by flash column chromatography with 220-g silica gel column, eluting with hexanes initially, grading to 40% ethyl acetate-hexanes, linear gradient to afford the desired product (1R,5S)-3-benzyl-8-(2-phenylpropan-2-yl)-3,8-diazabicyclo[3.2.1]oct-6-ene. MS (ESI), m/z (M+H)⁺ 319.

### Step C: (1R,5S)-3-benzyl-3,8-diazabicyclo[3.2.1]oct-6-ene (Int-BY3)

(1R,5S)-3-Benzyl-8-(2-phenylpropan-2-yl)-3,8-diazabicyclo[3.2.1]oct-6-ene (Int-BY2) (10.3 g, 32.5 mmol) and L-cysteine (11.8 g, 97.0 mmol) were added in a 250 mL two necked flask equipped with a reflux condenser. The reaction vessel was evacuated and backfilled with a balloon of nitrogen three times. DCM (54 mL) and TFA (54 mL) were added into the reaction vessel. The resulting mixture was heated at 50° C. for 1.5h. The product mixture was cooled down to room temperature and then poured into saturated sodium carbonate aqueous solution. After the mixture was thoroughly shook, the aqueous layer was basified to pH >14 with crushed sodium hydroxide solid. The aqueous layer was extracted three times with DCM (3×30 mL). The organic layers were dried over sodium sulfate. The dried solution was filtered and the filtrate was concentrated to dryness. The residue obtained containing (1R,5S)-3-benzyl-3,8-diazabicyclo[3.2.1]oct-6-en was used directly in the next step without purification.

### Step D: Tert-butyl (1R,5S)-3-benzyl-3,8-diazabicyelo[3.2.1]oct-6-ene-8-carboxylate (Int-BY4)

(1R,5S)-3-benzyl-3,8-diazabicyclo[3.2.1]oct-6-ene (Int-BY3) (6.51 g, 32.5 mmol) was added into a 500 mL flask. DCM (108 mL) and triethylamine (9.06 mL, 65.0 mmol) were added into the reaction vessel to dissolve the starting material. Di-tert-butyl dicarbonate (14.2 g, 65.0 mmol) and N,N-dimethylpyridin-4-amine (0.397 g, 3.25 mmol) were added into the reaction vessel. The resulting mixture was stirred for 1 h at 24° C. The product mixture was diluted with ether (400 mL) and washed three times with saturated sodium chloride aqueous solution (3×100 mL). The organic layer was dried over sodium sulfate. The dried solution was filtered and the filtrate was concentrated to dryness. The residue obtained was purified by flash column chromatography with 220-g silica gel column, eluting with hexanes initially, grading to 40% ethyl acetate-hexanes, linear gradient to afford the desired product tert-butyl (1R,5S)-3-benzyl-3,8-diazabicyclo[3.2.1]oct-6-ene-8-carboxylate. ¹H NMR (500 MHz, Chloroform-d) δ 7.29 (dq, J=14.1, 7.1 Hz, 5H), 6.20 (d, J=18.3 Hz, 2H), 4.52 (d, J=51.6 Hz, 2H), 3.60 (s, 2H), 2.61 (d, J=10.8 Hz, 2H), 2.38 (dd, J=52.1, 10.9 Hz, 2H), 1.46 (s, 9H). Step E: Tert-butyl 3-benzyl-1-methyl-3,8-diazabicyclo[3.2.1]oct-6-ene-8-carboxylate (Int-BY5)

Tert-butyl (1R,5S)-3-benzyl-3,8-diazabicyclo[3.2.1]oct-6-ene-8-carboxylate (Int-BY4) (9.74 g, 32.4 mmol) was added to a round bottomed flask. The reaction vessel was evacuated and back filled with nitrogen thrice then THF (195 mL) was added. Reaction mixture was cooled to −78° C. TMEDA (5.48 m:, 36.3 mmol) was added next. To this solution was added sec-butylthium (1.4 M, 34.7 mL, 48.6 mmol). The mixture was stirred for 60 min at −78° C. Then methyl iodide (3.04 mL, 48.6 mmol) was added neat. The reaction mixture was stirred at the same temperature for 1h. The product mixture was worked up by washing with saturated sodium bicarbonate solution (100 mL) and extracted with EtOAc (2×200 mL). The organic layers were dried with MgSO₄, concentrated to dryness and columned with EtOAc/Hex 0-100% to afford tert-butyl 3-benzyl-1-methyl-3,8-diazabicyclo[3.2.1]oct-6-ene-8-carboxylate. MS (ESI), m/z (M+H)⁺ 315.

### Step F: Tert-butyl 3-benzyl-1,5-dimethyl-3,8-diazabicyclo[3.2.1]oct-6-ene-8-carboxylate (Int-BY6)

Tert-Butyl 3-benzyl-1-methyl-3,8-diazabicyclo[3.2.1]oct-6-ene-8-carboxylate (Int-BY5) (7.19 g, 22.8 mmol) and N′, N′, N′, N²-tetramethylethane-1,2-diamine (3.87 mL, 25.6 mmol) were added into a 1 L round-bottomed flask. The reaction vessel was evacuated and backfilled with a balloon of nitrogen three times. THF (152 mL) was added into the reaction mixture. The resulting solution was cooled down to −78° C. A 0.7 M solution of sec-butylthium (49.0 mL, 34.3 mmol) was cannulated into the reaction mixture over 15 min. The resulting mixture was stirred for 1 h at −78° C. Then iodomethane (2.15 mL, 34.3 mmol) was added dropwise. The resulting mixture was stirred for 10 min at -78° C. then gradually warmed up to 0° C. The product mixture was quenched with ammonium chloride aqueous solution (50 mL). The layers that formed were separated. The organic layer was dried over sodium sulfate. The dried solution was filtered and the filtrate was concentrated to dryness. The residue obtained was purified by flash column chromatography with 220-g silica gel column, eluting with hexanes initially, grading to 50% ethyl acetate-hexanes, linear gradient to afford tert-butyl 3-benzyl-1,5-dimethyl-3,8-diazabicyclo[3.2.1]oct-6-ene-8-carboxylate. MS (ESI): m/z (M+H)⁺ 329.

### Step G: Tert-butyl (1R,5.6S)-3-benzyl-6-hydroxy-1.5-dimethyl-3,8-diazabicyclo[3.2.1]octane-8-carboxylate (Int-BY7-1) and tert-butyl (1S,5R,6R)-3-benzyl-6-hydroxy-1,5-dimethyl-3,8-diazabicyclo[3.2.1]octane-8-carboxylate (Int-BY7-2)

Tert-butyl 3-benzyl-1,5-dimethyl-3,8-diazabicyclo[3.2.1]oct-6-ene-8-carboxylate (Int-BY6) (1.95 g, 5.93 mmol) was added in a 40 mL vial. The reaction vessel was evacuated and backfilled with a balloon of nitrogen three times. THF (6.6 mL) was added into the reaction vessel to dissolve the starting material. Borane (14.8 mL, 29.6 mmol) in THF was added dropwise into the reaction vessel at 24° C. The resulting mixture was stirred for 16h at room temperature. Water (13 mL) was added dropwise very carefully into the reaction vessel (gas evolution). After the gas evolution ceased, sodium perborate tetrahydrate (4.56 g, 29.6 mmol) was added into the reaction vessel in one portion. The resulting mixture was stirred for 3h. The product mixture was extracted three times with DCM (3.30 mL) and three times with ether (3×30 mL). The organic layer was dried over sodium sulfate. The dried solution was filtered and the filtrate was concentrated to dryness. The residue obtained was purified by flash column chromatography with 24-g silica gel column, eluting with hexanes initially, grading to 80% ethyl acetate-hexanes, linear gradient. The material was further resolved by SFC (Lux-2, 21×250 mm, 5 um, Flow rate: 70 mL/min. Modifier: 10% MeOH w/0.1% NH₄OH) to afford tert-butyl (1R,5S,6S)-3-benzyl-6-hydroxy-1,5-dimethyl-3,8-diazabicyclo[3.2.1]octane-8-carboxylate as Peak 1, (Int-BY7-1) and tert-butyl (1S,5R,6R)-3-benzyl-6-hydroxy-1,5-dimethyl-3,8-diazabicyclo[3.2.1]octane-8-carboxylate as Peak 2, (Int-BY7-2). MS (EST): m/z (M+H)⁺ 347.

### Step H: Tert-butyl (1R,5MAS)-3-benzyl-6-(methoxymethoxy)-1.5-dimethyl-3,8-diazabicyelo[3.2.1]octane-8-carboxylate (Int-BY5-1)

Tert-Butyl (1R,5S,6S)-3-benzyl-6-hydroxy-1,5-dimethyl-3,8-diazabicyclo[3.2.1]octane-8-carboxylate (Int-BY7-1) (Peak 1, 214 mg, 0.618 mmol) was added in a 30 mL vial. DME (1.5 mL) was added into the reaction vessel to dissolve the starting material. N-Ethyl-N-isopropylpropan-2-amine (440 μL. 2.47 mmol), sodium iodide (93 mg, 0.62 mmol), and chloro(methoxy)methane (99 mg, 1.2 mmol) were added into the reaction vessel. The resulting mixture was stirred for 16h at 24° C. The product mixture was diluted with DCM (100 mL). The diluted product mixture was washed three times with saturated sodium bicarbonate aqueous solution (3 −20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 50% ethyl acetate-hexanes, linear gradient to afford tert-butyl (1R,5S,6S)-3-benzyl-6-(methoxymethoxy)-1,5-dimethyl-3,8-diazabicyclo[3.2.1]octane-8-carboxylate. MS (ESI): m/z (M+H)⁺ 391.

### Step 1: Tert-butyl (R,5S,6S)-6-(methoxymethoxy)-1,5-dimethyl-38-diazabicyclo[3.2.1]octane-8-carboxylate (Int-BY9-1)

Tert-butyl (1R,5S,6S)-3-benzyl-6-(methoxymethoxy)-1,5-dimethyl-3,8-diazabicyclo[3.2.1]octane-8-carboxylate (Int-BY8-1) (187 mg, 0.478 mmol) and palladium on activated charcoal (10 wt %, 25 mg, 0.024 mmol) were added in a 30 mL vial. The reaction vessel was evacuated and backfilled with a balloon of nitrogen three times. Methanol (4.8 mL) was added into the reaction vessel. The reaction vessel was evacuated and backfilled with a balloon of nitrogen three times. The reaction vessel was evacuated and backfilled with a balloon of hydrogen three times. The reaction mixture was stirred for 4h. The product mixture was thoroughly purged with nitrogen and filtered. The filtrate was concentrated to dryness. The residue containing tert-butyl (1R,5S,6S)-6-(methoxymethoxy)-1,5-dimethyl-3,8-diazabicyclo[3.2.1]octane-8-carboxylate obtained this way was used directly in the next step without purification.

### Intermediate BZ-2: 8-((triisonrooylsilyl)ethynyl)naphthalen-1-yl-23A.5.6.7-dg trifluoromethanesulfonate (Int-BZ-2)

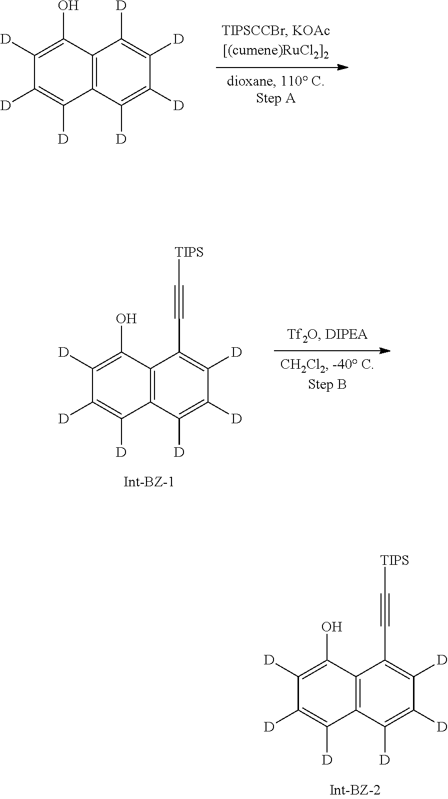

### Step A: 8-((triisoarouvisilyl)ethynyl)naphthalen-2,3,4,5,6,7-de-1-ol (Int-BZ-1)

To naphthalen-d₇-1-ol (400 mg, 2.65 mmol) and dichloro(p-cymene)ruthenium(II) dimer (162 mg, 0.265 mmol) and potassium acetate (519 mg, 5.29 mmol) in dioxane (4.4 mL), (bromoethynyl)triisopropylsilane (761 μL, 3.17 mmol) was added. The flask was thoroughly flushed with nitrogen and reaction stirred for 16h at 110° C. The reaction mixture was filtered through a bed of CELITE. The filter bed was washed with excess DCM. The filtrate was concentrated and directly purified on a silica gel column using 0-10% Hex-EtOAc to afford 8-((triisopropylsilyl)ethynyl)naphthalen-2,3,4,5,6,7-d₆-1-ol. ¹H NMR (500 MHz, Chloroform-4) δ 9.23 (d, J=1.6 Hz, 1H), 1.42-1.15 (m, 21H).

### Step B: 8-((triisopropylsilyl)ethynyl)naphthalen-1-yl-2,3,45.6.7-d trifluoromethanesulfonate (Int-BZ-2)

To 8-((triisopropylsilyl)ethynyl)naphthalen-2,3,4,5,6,7-d₆-1-ol (Int-BZ-1) (817 mg, 2.47 mmol) and DIPEA (1,30 mL, 7.41 mmol) in DCM (12 mL), triflic anhydride (626 μL, 3.71 mmol) was added at −40° C. and stirred for an hour. The reaction mixture was filtered through a bed of CELITE. The filter bed was washed with excess DCM. The filtrate was concentrated and directly purified on a silica gel column using 0-10% Hex- EtOAc to afford 8-((triisopropylsilyl)ethynyl)naphthalen-1-yl-2,3,4,5,6,7-d₆ trifluoromethanesulfonate. ¹H NMR (500 MHz., Acetonitrile-d₃) δ 1.36-1.00 (m, 21H).

### Intermediate CA5: 4-bromo-5-(chlorodifluoromethoxy)-6-methyl-1-(tetrahydro-2H-pyran-2-yl)-1H-indazole (Int-CA5)

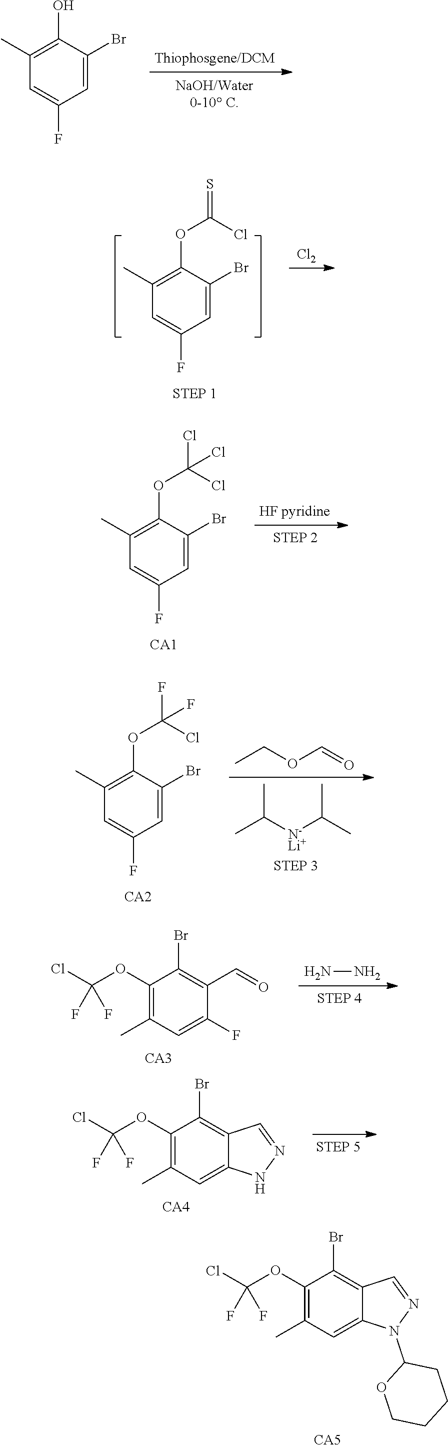

### STEP 1: 1-bromo-2-[(trichloro)methoxy]-5-fluoro-3-methyl-benzene (Int-CA1)

To a flask fitted with 2 base scrubbers was added 5% NaOH (1.1 eq) followed by 2-bromo-4-fluoro-6-methyl-phenol (1.0 eq). The resulting solution was stirred at rt for 20 min before being cooled to 0° C. Thiophosgene (1.0 eq) dissolved in DCM (4 vol) was added at a steady rate maintaining the temperature below 10° C. Once addition was complete, the reaction mixture was stirred for 2 hr maintaining the temperature below 10° C. at which point ¹H NMR (ipc mini w/u: RM washed with 2M HCl and water) indicated the reaction to be complete. The orange suspension was filtered, and the filter cake washed with DCM (3.5 vol). The filtered layers were separated and the aqueous extracted with DCM (3 vol). The combined organics were washed with 2M HCl (3.5 vol) followed by a water wash (3.5 vol). The organic layer was dried, filtered and transferred to a suitable sized flask, equipped with a gas outlet to 2×base scrubbers. The orange solution was cooled to 0° C. and treated with C₁₂ gas (6 eq) keeping the internal temperature below 8° C. The reaction mixture was stirred and allowed to slowly warm to rt over 16 hr. GC indicated the reaction to be complete. The reaction was purged with nitrogen bubbled through the reaction mixture for 60 min before the bulk of the solvent was removed under reduced pressure (water bath 50° C.) to give crude 1-bromo-2-[(trichloro)methoxy]-5-fluoro-3-methyl-benzene.

No starting material is observable by ‘H or’F NMR. GC analysis shows that the starting material has been consumed and a new peak is observed at 12.70 min. ¹H NMR (DMSO) δ 7.22-7.20 (m, 1H), 6.94-6,91 (m, 1H), 2.49 (s, 3H).

### STEP 2: 1-bromo-2-(chlorodifluoromethoxy)-5-fluoro-3-methylbenzene (Int-CA2,)

4-bromo-2-1(trichloro)methoxyl-5-fluoro-3-methyl-benzene (Int-CA1) was reacted with HF pyridine in a suitable size vessel (equipped with stirrer and connected to a KOH and water scrubbers). The resulting mixture was stirred for 18 hr at 45° C. and ion pair chromatography (IPC) (quenched sample in ice/water and extract with DCM. The organic phase was neutralized with saturated aqueous sodium bicarbonate and analysis was done using GC and NMR) showed no starting material remained. The reaction mixture was quenched in ice/water (6 vol) and extracted with DCM (2×3 vol). The organic layers were dried over anhydrous magnesium sulphate. The solvent was removed under vacuo to give a crude product. (intermittent ¹⁹F analysis showed integration between the fluorine peaks at 53.8 ppm and 37.16 ppm is 1:0.57 indicating the reaction was not complete). Crude material collected after work up was purified by vacuum distillation giving 1-bromo-2-(chlorodifluoromethoxy)-5-fluoro-3-methylbenzene. GC-MS- 9.38 mins area -97.74%. ⁹F NMR S -53.84, -37.16.

### STEP 3: 2-bromo-3-(chlorodifluoromethoxy)-6-fluoro-4-methylbenzaldehyde (Int-CA3)

To a solution of 1-bromo-2-(chlorodifluoromethoxy)-5-fluoro-3-methylbenzene (1 g, 3.45 mmol) (Int-CA2) in THF (10 ml) was added lithium diisopropylamide (4.15 ml, 4.15 mmol) at −78° C. under N2 atmosphere. The mixture was stirred at −78° C. for 1 h. Then ethyl formate (0.281 g, 3.80 mmol) was added at −78° C. and was stirred at −78° C. for 0.5h. The mixture was diluted with aq. NH₄Cl (5 mL) and extracted with EtOAc (3×10 mL), dried over sodium sulfate, filtered and the solvent was evaporated under reduced pressure to give the crude product. The crude product was purified by flash silica gel chromatography 4 g, Eluent of PE gradient to give 2-bromo-3-(chlorodifluoromethoxy)-6-fluoro-4-methylbenzaldehyde.

### STEP 4: 4-bromo-5-(chlorodifluoromethoxy)-6-methyl-1H-indazole (Int-CA4)

To a solution of 2-bromo-3-(chlorodifluoromethoxy)-6-fluoro-4-methylbenzaldehyde (0.9 g, 2.83 mmol) (Int-CA3) in DMSO (9 ml) was added hydrazine (0.534 g, 14.17 mmol) at 20° C. under N2. The mixture was stirred at 120° C. for 12 h. The mixture was diluted with water (10 mL) and extracted with EtOAc (3×30 mL). The organic layer was washed with water (3×10 mL), dried over sodium sulfate, filtered and the solvent was evaporated under reduced pressure to give the crude product. The crude product was purified by flash silica gel chromatography 4 g, eluent of 0-10% EA/PE gradient to give 4-bromo-5-(chlorodifluoromethoxy)-6-methyl-1H-indazole.

### STEP 5: 4-bromo-5-(chlorodifluoromethoxy)-6-methyl-1-(tetrahydro-2H-pyran-2-yl)-1H-indazole (Int-CA5)

To a solution of 4-bromo-5-(chlorodifluoromethoxy)-6-methyl-1H-indazole (630 mg, 2.022 mmol) (Int-CA4) in dioxane (7 ml) was added pyridine 4-methylbenzenesulfonate (102 mg, 0.404 mmol) and 3.4-dihydro-2H-pyran (340 mg, 4.04 mmol) at 20° C. The mixture was stirred at 90° C. for 12 h. The mixture was diluted with water (4 mL) and extracted with EtOAc (3×10 mL), dried over sodium sulfate, filtered and the solvent was evaporated under reduced pressure to give the crude product. The crude product was purified by flash silica gel chromatography 4 g, eluent of 0-20% EA/PE gradient to give 4-bromo-5-(chlorodifluoromethoxy)-6-methyl-1-(tetrahydro-2H-pyran-2-yl)-1H-indazole. MS (ESI): m/z [M+H+2]⁺: 394, 396.

### Intermediate CB7: 4-bromo-5-(difluoromethyl)-6-methyl-1-(tetrahydro-2H-pyran-2-yl)-1H-indazole (Int-CB7)

### Scheme:

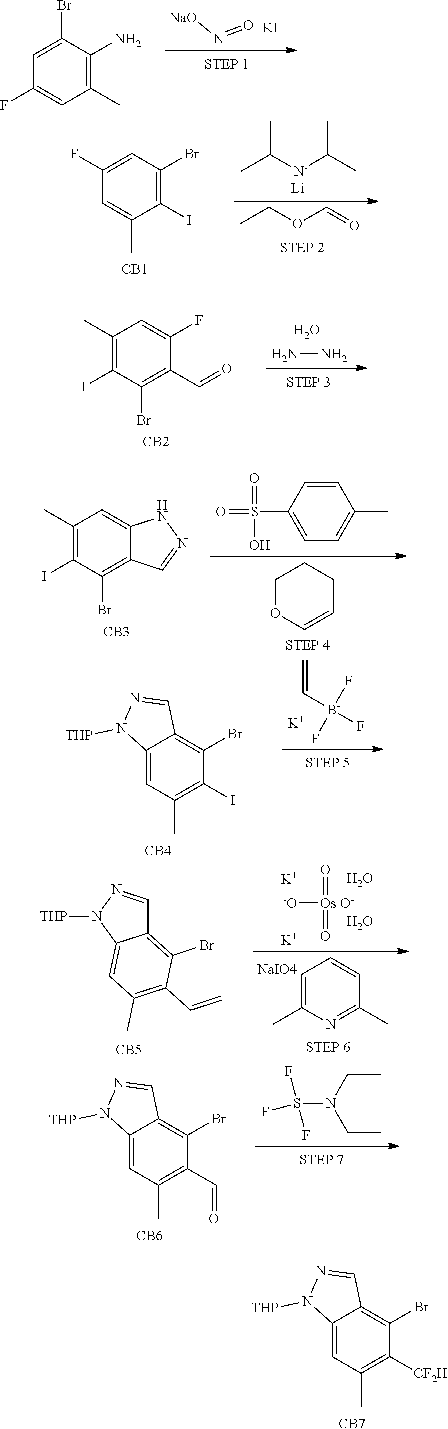

### STEP 1: 1-bromo-5-fluoro-2-iodo-3-methylbenzene (Int-CBI) PP14C

A solution of 2-bromo-4-fluoro-6-methylaniline (Int-AAA1) (20 g, 98 mmol) in 6M HCl (200 ml) was stirred at 70° C. for 1 h. Then sodium nitrite (8.12 g, 118 mmol) in water (50 ml) was added to the reaction mixture at −10° C., and the resulting mixture was stirred at −10° C. for 30 min. Then potassium iodide (19.53 g, 118 mmol) in water (80 ml) was added to the mixture at −10° C. for 10 min. The reaction mixture was diluted with EtOAc (2× 200 mL) at 0° C., and the resulting mixture was washed with brine (300 mL×3). The combined organic phase was dried over anhydrous Na₂SO₄, filtered and concentrated in vacuo. The residue was purified by flash silica gel chromatography 120 g column, Pet.ether/EtOAc=20/1 to give 1-bromo-5-fluoro-2-iodo-3-methylbenzene. ¹H NMR (400 MHz, CHLOROFORM-d) δ=7.30-7.23 (m, 1H), 6.96 (dd, J=2.9, 8.9 Hz, 1H), 2.57 (s, 3H).

### STEP 2: 2-bromo-6-fluoro-3-iodo-4-methylbenzaldehyde (Int- CB2)

To a solution of diisopropylamine (15 ml, 106 mmol) in THF (45 ml) was added nBuLi (40 ml, 100 mmol)(2.5M in hexanes) at −78° C., and the mixture was stirred at −78° C. for 30 min. The solution was assumed as lithium diisopropylamide (100 ml, 1 M in THF).

To a solution of 1-bromo-5-fluoro-2-iodo-3-methylbenzene (24 g, 76 mmol) (Int-CB1) in THF (45 ml) was added the above prepared 1 M lithium diisopropylamide (91 ml, 91 mmol) at −78° C. under N2. The mixture was stirred at −78° C. for 0.5 h. Then ethyl formate (6.21 g, 84 mmol) was added at −78° C. and was stirred at −78° C. for 15 min. The mixture was slowly poured into aq. NH₄Cl (200 mL) and extracted with EtOAc (3×400 mL), dried over sodium sulfate, filtered and the solvent was evaporated under reduced pressure to give the crude product. The crude product was purified by flash silica gel chromatography 220 g column, eluent of 5% EA/PE gradient to give 2-bromo-6-fluoro-3-iodo-4-methylbenzaldehyde. ¹H NMR (400 MHz, CHLOROFORM-d) S =10.16-10.02 (m, 1H), 7.05 (d, J=11.0 Hz, 1H), 2.56 (s, 3H).

### STEP 3: 4-bromo-5-iodo-6-methyl-1H-indazole (Int-CB3)

To a solution of 2-bromo-6-fluoro-3-iodo-4-methylbenzaldehyde (18.7 g, 54.5 mmol) (Int-CB2) in DMSO (200 ml) was added hydrazinium hydroxide (19.84 ml, 327 mmol)(85% aqeuous solution) at 20° C. under N₂. The reaction mixture was stirred at 120° C. for 12 h. The reaction mixture was poured into ice water (50 mL) and extracted with EtOAc (3×400 mL). The organic layer was washed with aq. NaCl (2×50 mL), dried over sodium sulfate, filtered and the solvent was evaporated under reduced pressure to give 4-bromo-5-iodo-6-methyl-1H-indazole, which was used in the next step without further purification. MS (ESI): m/z (M+H)⁺ 336,338.

### STEP 4: 4-bromo-5-iodo-6-methyl-1-(tetrahydro-2H-pyran-2-yl)-1H-indazole (Int-CB4)

To a solution of 4-bromo-5-iodo-6-methyl-1H-indazole (15.5 g, 46.0 mmol) (Int-CB3) in THF (200 ml) was added 4-methylbenzenesulfonic acid (1.584 g, 9.20 mmol) and DHP (8.41 ml, 92 mmol) at 20° C., and the mixture was stirred at 50° C. for 12 h. The reaction mixture was concentrated in vacuo, and the residue was purified by flash silica gel chromatography 120 g column, eluent of 0-15% EA/PE gradient to give 4-bromo-5-iodo-6-methyl-1-(tetrahydro-2H-pyran-2-yl)-1H-indazole. The white solid was diluted with MeOH(50 mL), and the resulting mixture was filtered. The filtered solid was collected to afford to get 4-bromo-5-iodo-6-methyl-1-(tetrahydro-2H-pyran-2-yl)-1H-indazole. MS (ESI), m/z [M+H+2]⁺: 421,423. ¹H NMR (400 MHz, CHLOROFORM-d) δ=7.84 (s, 1H), 7.44-7.35 (m, 1H), 5.58 (dd, J=2.7, 9.1 Hz, 1H), 4.00-3.85 (m, 1H), 3.73-3.59 (m, 1H), 2.67-2.58 (m, 3H), 2.52-2.36 (m, 1H), 2.16-1.93 (m, 2H), 1.76-1.51 (m, 3H).

### STEP 5: 4-bromo-6-methyl-1-(tetrahydro-2H-pyran-2-yl)-5-vinyl-1H-indazole (Int-CB5)

To a solution of 4-bromo-5-iodo-6-methyl-1-(tetrahydro-2H-pyran-2-yl)-1H-indazole (6 g, 14.25 mmol) (Int-CB4) in tert-amyl alcohol (60.000 ml) and Water (20 ml) was added potassium vinyltrifluoroborate (2.481 g, 18.52 mmol), Cs₂CO₃ (13.93 g, 42.7 mmol) and Pd(Ph₃P)₄ (0.823 g, 0.712 mmol) at 25° C. The mixture was stirred at 100° C. for 16 h. The combined reaction mixture was extracted with EtOAC (200×3 mL), dried over Na₂SO₄, filtered and the solvent was evaporated under reduced pressure to give the crude product. The crude product was purified by flash silica gel chromatography 120 g column, eluent of 0-15% EA/PE gradient to give 4-bromo-6-methyl-1-(tetrahydro-2H-pyran-2-yl)-5-vinyl-1H-indazole. MS (ESI): m/z (M+H)⁺ 321, 323.

### STEP 6: 4-bromo-6-methyl-1-(tetrahydro-2H-pyran-2-yl)-1H-indazole-5-carbaldehyde (Int-CB6)

To a solution of 4-bromo-6-methyl-1-(tetrahydro-2H-pyran-2-yl)-5-vinyl-1H-indazole (4.5 g, 12.89 mmol) (Int-CB5) in THF (50 ml) and water (50 ml) was added 2,6-dimethylpyridine (2.76 g, 25.8 mmol), sodium periodate (11.03 g, 51.6 mmol) and potassium osmate (VI) dihydrate (0.475 g, 1.289 mmol) at 25° C., and the mixture was stirred at 50° C. for 2 h. The reaction was quenched with the saturated solution of NaSO₃ (20 mL) and the mixture was extracted with EtOAc (200 mL×3). The combined organic layers were washed with brine (20 mL), dried over anhydrous Na₂SO₄, filtered and concentrated. The residue was purified by flash silica gel chromatography 120 g, Pet.ether/EtOAc=5/1 to give 4-bromo-6-methyl-1-(tetrahydro-2H-pyran-2-yl)-1H-indazole-5-carbaldehyde. LCMS/(M+H+): 323,325. ¹H NMR (400 MHz, CHLOROFORM-d) 8=10.62-10.44 (m, 1H), 8.09 (s, 1H) 7.30 (s, 1H) 5.62 (dd, J=2.8, 9.1 Hz, 1H), 4.02-3.85 (m, 1H), 3.69 (ddd, J=3.0, 10.0, 11.6 Hz, 1H), 2.66 (d, J=0.7 Hz, 3H), 2.51-2.34 (m, 1H), 2.17-1.92 (m, 2H), 1.78-1.56 (m. 3H).

### STEP 7: 4-bromo-5-(difluoromethyl)-6-methyl-1-(tetrahydro-2H-pyran-2-yl)-1H-indazole (Int-CB7)

To a solution of 4-bromo-6-methyl-1-(tetrahydro-2H-pyran-2-yl)-1H-indazole-5-carbaldehyde (650 mg, 2.011 mmol) (Int-CB6) in DCM (10 ml) was added DAST (1.860 ml, 14.08 mmol) at 0° C. under N2 and the mixture was stirred at 25° C. for 12 h. The reaction mixture was added dropwise to NaHCO₃(20 mL), and the mixture was extracted with EtOAc(120 mL×3). The combined organic phase was dried over anhydrous Na₂SO₄, filtered and concentrated in vacuo. The residue was purified by flash silica gel chromatography 20 g, Pet.ether/EtOAc=5/l to give 4-bromo-5-(difluoromethyl)-6-methyl-1-(tetrahydro-2H-pyran-2-yl)-1H-indazole. MS (ESI): m/z (M+H)⁺ 345, 347.

### Preparation of Final Compounds

### Example 1: 3-(6-chloro-8-fluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 1a)

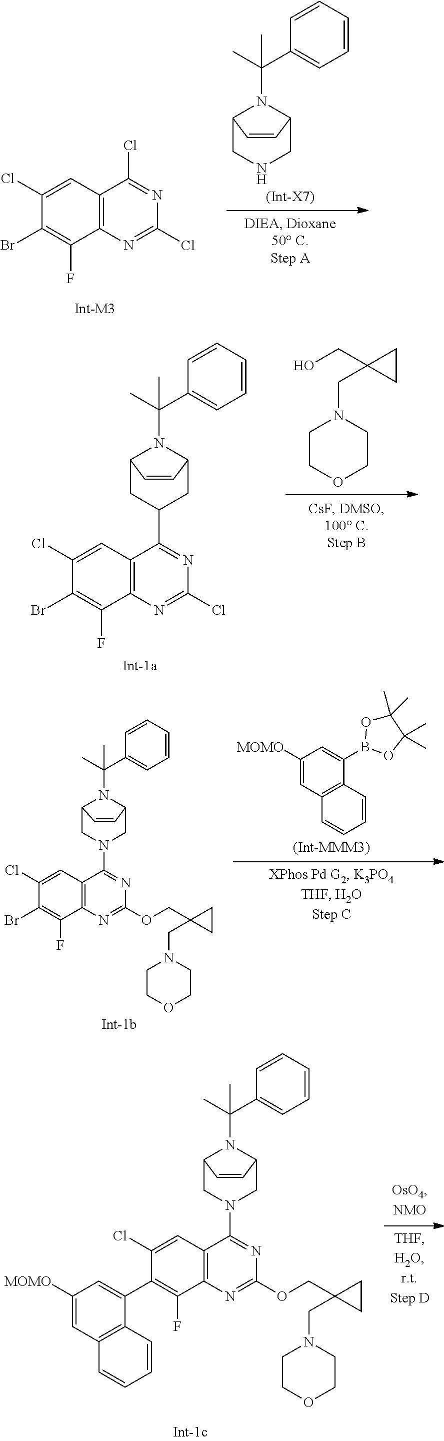

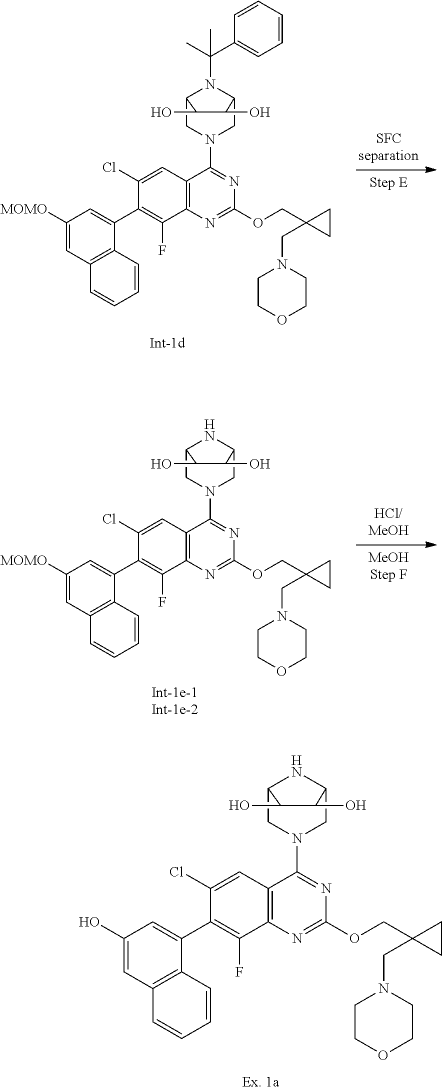

mmol). The mixture was heated to 50° C. for 30 min After 30 minutes, the mixture was allowed to cool to room temperature. The mixture was diluted with water (50 mL) and extracted with EtOAc (50 mL×2). The combined organic layers were dried over Na₂SO₄, filtered and concentrated in vacuo. The resulting residue was purified by flash silica gel chromatography (Eluent of 0--100% DCM gradient in petroleum ether) to afford 7-bromo-2, 6-dichloro-8-fluoro-4-(8-(2-phenylpropan-2-yl)-3,8-diazabicyclo[3.2.1]oct-6-en-3-yl)quinazoline (Int-1a). MS (ESI): m/z (M+H)⁺ 521/523.

### Step B: 4-((1-(((7-bromo-6-chloro-8-fluoro-4-(8-(2-phenylpropan-2-Y1)-3,8-diazabicyclo[3.2.1]oct-6-en-3-yl)quinazolin-2-yl)oxy)methyl)cycloyroyl)methyl)moryholine (Int-1b)

To a mixture of 7-bromo-2,6-dichloro-8-fluoro-4-(8-(2-phenylpropan-2-yl)-3,8-diazabicyclo[3.2.1]oct-6-en-3-yl)quinazoline (Int-1α) (0.46 g, 0.88 mmol) in DMSO (8.0 mL) were added (1-(morpholinomethyl)cyclopropyl)methanol (0.38 g, 2.2 mmol) and CsF (0.54 g, 3.5 mmol) in a glove box. The mixture was heated to 100° C. for 3 h. After 3 h. the mixture was allowed to cool to room temperature, diluted with water (50 mL) and then extracted with EtOAc (50 mL×2). The combined organic layers were dried over Na₂SO₄, filtered and concentrated in vacuo. The residue was purified by flash silica gel chromatography (Eluent of 0-50% EtOAc gradient in Pet, ether) to afford 4-((1-(((7-bromo-6-chloro-8-fluoro-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-1b). MS (ESI): m/z (M+H)⁺ 656/658.

### Step C: 4-((1-((6-chloro-8-fluoro-7-(3-(methoxymethoxy)naphthalen-1-yl)-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-1c)

To a mixture of 4-((1-(((7-bromo-6-chloro-8-fluoro-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-1b) (0.38 g, 0.57 mmol) and 2-(3-(methoxymethoxy)naphthalen-1-yl)-4,4,5,5-tetramethyl-1,3,2-dioxaborolane (Int-MMM3) (0.23 mg, 0.74 mmol) in THF (12 mL) were added potassium phosphate tribasic (1.5 M in water, 0.95 mL, 1.4 mmol) followed by chloro(2-dicyclohexylphosphino-2′,4′,6′-triisopropyl-1,1′-biphenyl)[2-(2′-amino-1,1′-biphenyl)]palladium(II) (Xphos-Pd-G2) (45 mg, 0.057 mmol) under a N₂ atmosphere. Then the mixture was stirred at 20° C. for 2 h. The mixture was then dissolved in water (40 mL) and extracted with EtOAc (40 mL×2). The combined organic layers were dried over Na₂SO₄, filtered and concentrated in vacuo. The resulting residue was purified by flash silica gel chromatography (eluent of 10-70% EtOAc gradient in Pet, ether) to afford 4-((1-(((6-chloro-8-fluoro-7-(3-(methoxymethoxy)naphthalen-1-yl)-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-1c). MS (ESI): m-: (M+H)⁺ 764.

### Step D: 3-(6-chloro-8-fluoro-7-(3-(methoxymethoxy)naphthalen-1-yl)-2-((I-(morpholinomethyl)cyclopropyl)methoxy)quinazolin-4-yl)-842-phenylpropan-2-yl)-3,8-diazabicyclo[3.2.1]octane-6,7-diol (Int-1d)

To a mixture of 4-((1-(((6-chloro-8-fluoro-7-(3-(methoxymethoxy)naphthalen-1-yl)-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-1c) (0.30 g, 0.39 mmol) in THF (9.0 mL) and water (3.0 mL) were added NMO (92 mg, 0.79 mmol) and O₃04 (4.0 wt % in water, 0.012 mL, 0.039 mmol). The mixture was stirred at 20° C. for 16 h. The mixture was then diluted in water (40 mL) and saturated Na₂S204 aqueous (10 mL). The resulting mixture was extracted with EtOAc (50 mL×2). The combined organic layers were dried over Na₂SO₄, filtered and concentrated in vacuo. The resulting residue was purified by reverse phase preparatory HPLC (MeCN/water with 0.1% TFA modifier) to afford 3-(6-chloro-8-fluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-1d). MS (ESI): m/z (M+H)⁺ 798.

### Step E: 3-(6-chloro-8-fluoro-7-(3-(methoxymethoxy)naphthalen-1-yl)-2-((1-(morpholinomethyl)cyclopropyl)methoxy)quinazolin-4-yl)-3,8-diazabicyclo[3.2.1]octane-6,7-diol (Int-le-1 and Int-le-2)

The racemic 3-(6-chloro-8-fluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-1d) (80 mg, 0.10 mmol) was separated by preparative SFC (Column A: Condition: 0.1% NH₄OH in EtOH) to afford 3-(6-chloro-8-fluoro-7-(3-(methoxymethoxy)naphthalen-1-yl)-2-((1-(morpholinomethyl)cyclopropyl)methoxy)quinazolin-4-yl)-3,8-diazabicyclo[3.2.1]octane-6,7-diol (Int-le-1, Peak 1) and 3-(6-chloro-8-fluoro-7-(3-(methoxymethoxy)naphthalen-1-y)-2-((1-(morpholinomethyl)cyclopropyl)methoxy)quinazolin-4-yl)-3,8-diazabicyclo[3.2.1]octane-6,7-diol (Int-le-2, Peak 2). The cumyl group was cleaved while waiting for SFC separation. MS (ESI): m z (M+H)⁺ 680.

### Step F: 3-(6-chloro-8-fluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 1a)

To a stirred solution of 3-(6-chloro-8-fluoro-7-(3-(methoxymethoxy)naphthalen-1-yl)-2-((1-(morpholinomethyl)cyclopropyl)methoxy)quinazolin-4-yl)-3,8-diazabicyclo[3.2.1]octane-6,7-diol (25 mg, 0.037 mmol) (Int-le-1) in MeOH (1.0 mL) was added HCl in methanol (4.0 M, 2.0 mL). The mixture was stirred at 20° C. for 20 min. The mixture was then concentrated in vacuo and the residue was purified by Prep-HPLC (MeCN/water with 0.2% formic acid modifier)) to afford 3-(6-chloro-8-fluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 1a). MS (ESI): m/z (M+H)⁺ 636. ¹H NMR (500 MHz, methanol-d₆) δ 8.03-7.97 (m, 1H), 7.78 (d, J=8.4 Hz, 1H), 7.47-7.41 (m, 1H), 7.29 (d, J=2,3 Hz, 1H), 7.25-7.18 (m, 2H), 7.05 (d, J=2.4 Hz, 1H), 4.70-4.62 (m, 2H), 4.51-4.42 (m, 4H), 3.93-3.85 (m, 6H), 3.72 (br d, J=13.6 Hz, 2H), 3.22-3.04 (m, 6H), 0.94-0.87 (m, 2H), 0.81-0.71 (m, 2H).

### Example 2: 3-(6.8-difluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 2)

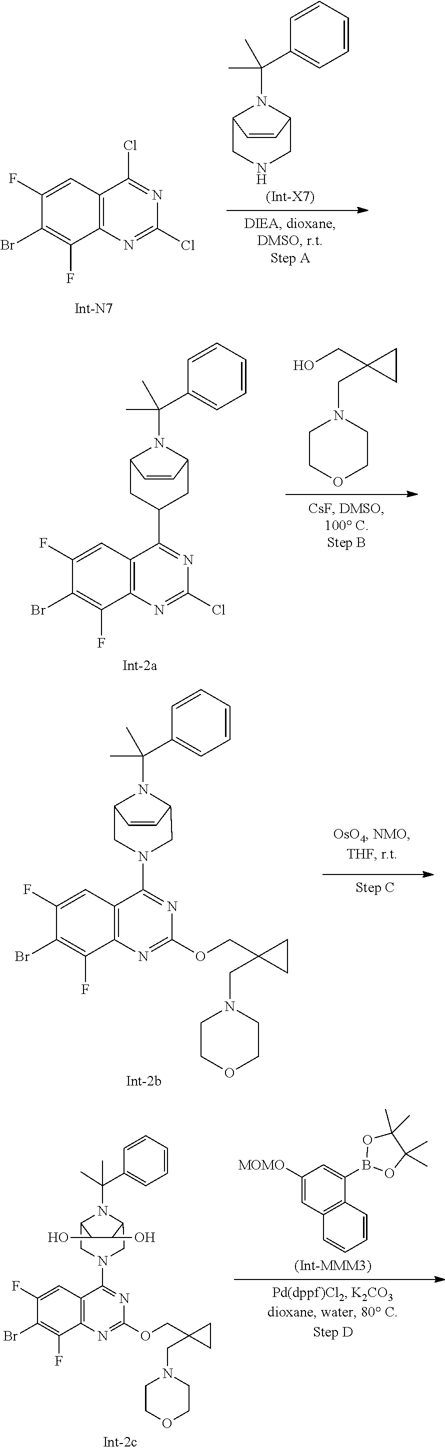

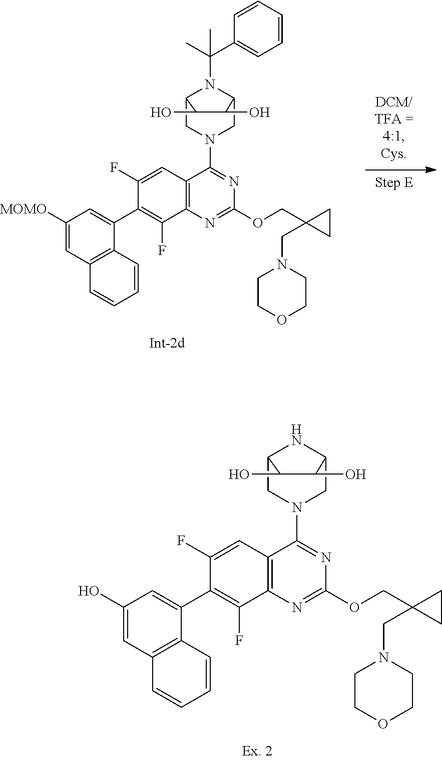

### Step A: 7-bromo-2-chloro-6,8-difluoro-4-(8-(2-phenylpropan-2-yl)-3,8-diazabicyclo[3.2.1]oct-6-en-3-yl)quinazoline (Int-2a)

To a mixture of 8-(2-phenylpropan-2-yl)-3,8-diazabicyclo[3.2.1]oct-6-ene (Int-X7) (0.40 g, 1.8 mmol) in dioxane (10 mL) were added DIEA (1.4 mL, 8.0 mmol) and 7-bromo-2,4-dichloro-6,8-difluoroquinazoline (Int-N7) (0.50 g, 1.6 mmol). The mixture was stirred at 20° C. for 12 h. The mixture was then dissolved in water (10 mL) and extracted with EtOAc (10 mL×3). The combined organic layers were dried over Na₂SO₄., filtered and concentrated in vacuo. The resulting residue was purified by flash silica gel chromatography (Eluent of 0-20% EtOAc gradient in Pet, ether) to afford 7-bromo-2-chloro-6,8-difluoro-4-(8-(2-phenylpropan-2-yl)-3,8-diazabicyclo[3.2.1]oct-6-en-3-yl)quinazoline (Int-2a). MS (ESI): m/z (M+H)⁺ 505/507.

### Step B: 4-((1-(((7-bromo-6,8-difluoro-4-(8-(2-phenylpropan-2-yl)-3,8-diazabicyclol[3.2.1]oct-6-en-3-yl)quinazolin-2-yl)oxy)methyl)cycloyropyl)methyl)morpholine (Int-2b)

To a stirred mixture of 7-bromo-2-chloro-6,8-difluoro-4-(8-(2-phenylpropan-2-yl)-3,8-diazabicyclo[3.2.1]oct-6-en-3-yl)quinazoline (Int-2a) (0.58 g, 1.1 mmol) and (1-(morpholinomethyl)cyclopropyl)methanol (0.49 g, 2.9 mmol) in dioxane (6.0 mL) and DMSO (2.0 mL) was added CsF (0.70 g, 4.6 mmol) at 20° C. under N₂. The mixture was then heated to 100° C. for 3 h. After 3 hours, the mixture was allowed to cool to room temperature and then water (20 mL) was added. The mixture was extracted with EtOAc (8 mL×3). The combined organic layers were dried over Na₂SO₄, filtered and concentrated in vacuo. The resulting residue was purified by flash silica gel chromatography (Eluent of 0-40% EtOAc gradient in Pet, ether) to afford 4-((1-(((7-bromo-6,8-difluoro-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-2b). MS (ESI): m/z (M+H)⁺ 640/642.

### Step C: 3-(7-bromo-6,8-difluoro-2-((1-(morpholinomethy)cyclopropyl)methoxy)quinazolin-4-yl)-8-(2-phenylpropan-2-V1)-3,8-diazabicyclo[3.2.1]octane-6,7-diol (Int-2c)

To a mixture of 4-((1-(((7-bromo-6,8-difluoro-4-(8-(2-phenylpropan-2-yl)-3,8-diazabicyclo[3.2.1]oct-6-en-3-yl)quinazolin-2-yl)oxy)methyl)cyclopropyl)methyl)morpholine (Int-2b) (0.20 g, 0.31 mmol) in THF (5.0 mL) were added osmium tetroxide (4.0 wt % in water, 49 μL, 0.16 mmol) and NMO (37 mg, 0.31 mmol) at 20° C., and the mixture was stirred at 20° C. for 3 h. The reaction mixture was then quenched with saturated Na₂SO₃ solution (2.0 mL) and partitioned between EtOAc (10 mL×2) and water (10 mL). The combined organic phases were dried over anhydrous Na₂SO₄, filtered and concentrated in vacuo. The residue was purified by Prep-TLC (SiO₂. DCM/MeOH=20:1) to afford 3-(7-bromo-6,8-difluoro-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-2c). MS (ESI): m/z (M+H)⁺ 674/676.

### Step D: 3-(6,8-difluoro-7-(3-(methoxymethoxyl)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-2d)

To a stirred mixture of 2-(3-(methoxymethoxy)naphthalen-1-yl)-4,4,5,5-tetramethyl-1,3,2-dioxaborolane (Int-MMM3) (23 mg, 0.074 mmol) and 3-(7-bromo-6,8-difluoro-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-2c) (50 mg, 0.074 mmol) in dioxane (1.5 mL) and water (0.30 mL) were added PdCl₂(dppf) (11 mg, 0.015 mmol) and Na₂CO₃ (20 mg, 0.19 mmol) at 20° C. under N2. The mixture was then heated to 80° C. for 2 h. After 2 hours, the mixture was allowed to cool to room temperature and was then concentrated in vactuo. The resulting residue was purified by prep-TLC (SiO₂, Pet, ether/EtOAc=1:2) to afford 3-(6,8-difluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-2d). MS (ESI): m/z (M+H)⁺ 782.

### Step E: 3-(6,8-difluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 2)

To a stirred mixture of 3-(6,8-difluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octane-6,7-diol (Int-2d) (25 mg, 0.032 mmol) in CH₂Cl₂ (4.0 mL) were added cysteine (7.8 mg, 0.064 mmol) and TFA (1.0 mL) at 20° C. The resulting mixture was stirred at 20° C. for 3 h. The residue was then purified by reverse phase Prep-HPLC (MeCN/water with 0.1% TFA modifier) to afford 3-(6,8-difluoro-7-(3-hydroxynaphthalen-1-yl)-2-((1-(morpholinomethyl)cyclopropyl)methoxy)quinazolin-4-yl)-3,8-diazabicyclo[3.2.1]octane-6,7-diol (Ex. 2) MS (ESI): m/z (M+H)⁺ 620. ¹H NMR (400 MHz, methanol-d₄) δ 7.78 (d, J=8.4 Hz, 1H), 7.70 (br d.. =9.6 Hz, 1H), 7.44 (t, J=7.6 Hz, 1H), 7.38-7.18 (m, 3H), 7.12 (d, J=2.4 Hz, 1H), 4.69-4.59 (m, 3H), 4.54-4.38 (m, 3H), 4.23-4.02 (m, 4H), 3.97-3.67 (m, 6H), 3.38 (s, 2H), 3.20 (br s, 2H), 0.99 (s, 2H), 0.89 (s, 2H).

### Example 3: 4-(6.8-difluoro-446-fluoro-3,8-diazabicyclo[3.2.1]octan-3-yl)-2-((I

(morpholinomethyl)cyclopropyl)methoxy)quinazolin-7-yl)naphthalen-2-ol (Ex. 3a/b 1

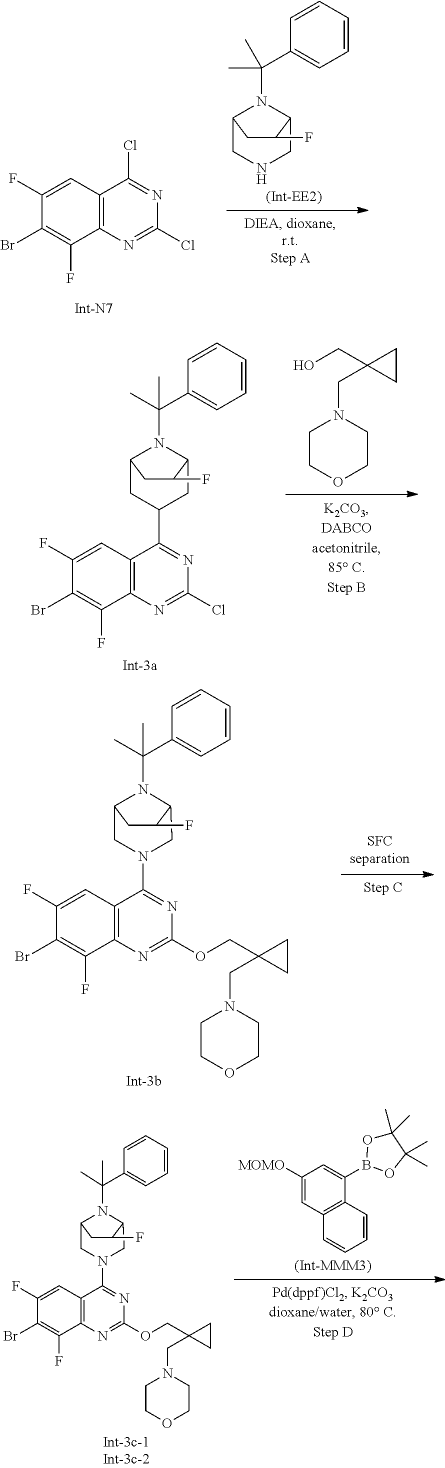

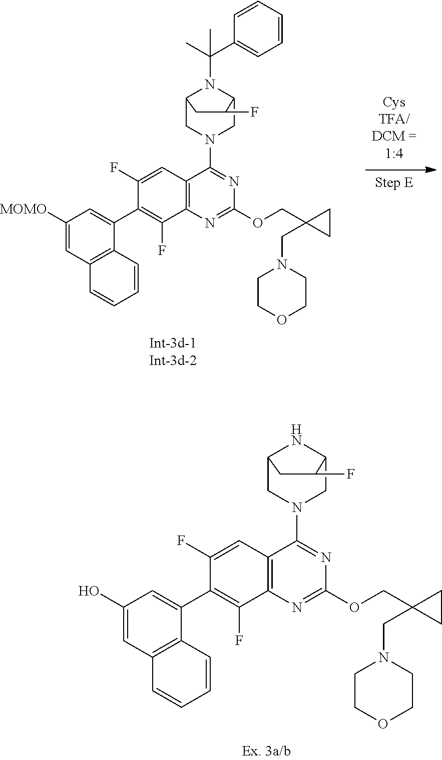

### Step A: 7-bromo-2-chloro-6,8-difluoro-4-(6-fluoro-8-(2-phenylpropan-2-yl)-3,8-diazabicyclo[3.2.1]octan-3-yl)quinazoline (Int-3a)

To a mixture of 6-fluoro-8-(2-phenylpropan-2-yl)-3,8-diazabicyclo[3.2.1]octane (Int-EE2) (0.12 g, 0.49 mmol) in dioxane (2.0 mL) were added DIEA (0.43 mL, 2.4 mmol) and 7-bromo-2,4-dichloro-6,8-difluoroquinazoline (Int-N7) (0.16 g, 0.50 mmol) at 20° C. under a N₂ atmosphere. The mixture was stirred at 20° C. for 3 h. The mixture was then diluted with water (3.0 mL) and extracted with EtOAc (3×4.0 mL). The combined organic layers were dried over Na₂SO₄, filtered and the solvent was evaporated under reduced pressure to afford the crude product. The crude product was purified by flash silica gel chromatography (Eluent of 0-62% EtOAc gradient in Pet, ether) to afford 7-bromo-2-chloro-6,8-difluoro-4-(6-fluoro-8-(2-phenylpropan-2-yl)-3,8-diazabicyclo[3.2.1]octan-3-yl)quinazoline (Int-3a). MS (ESI): m/z (M+H)⁺ 527.

### Step B: 4-((1-(((7-bromo-6,8-difluoro-4-(6-fluoro-8-(2-phenylpropan-2-v 1)-3,8-diazabicyclo[3.2.1]octan-3-yl)quinazolin-2-yl)oxy)methyl)cyclopropyl)methyl)morpholine (Int-3b)

To a stirred mixture of 7-bromo-2-chloro-6,8-difluoro-4-(6-fluoro-8-(2-phenylpropan-2-yl)-3,8-diazabicyclo[3.2.1]octan-3-yl)quinazoline (Int-3a) (30 mg, 0.057 mmol) and (1-(morpholinomethyl)cyclopropyl)methanol (20 mg, 0.11 mmol) in acetonitrile (0.50 mL) were added K₂CO₃ (24 mg. 0.17 mmol) and 1.4-diazabicyclo[2,2,2]octane (0.64 mg, 5.7 μmol) at 20° C. under a N₂ atmosphere. The mixture was then heated to 85° C. for 3 h. After 3 hours, the mixture was allowed to cool to room temperature and diluted with water (2.0 mL). The mixture was extracted with EtOAc (3.0 mL×3). The combined organic layers were dried over Na₂SO₄, filtered and the mixture was concentrated under reduced pressure to afford the crude product. The resulting residue was purified by preparative TLC (SiO₂, petroleum ether:ethyl acetate=5:1) to afford 4-((1-(((7-bromo-6,8-difluoro-4-(6-fluoro-8-(2-phenylpropan-2-yl)-3,8-diazabicyclo[3.2.1]octan-3-yl)quinazolin-2-yl)oxy)methyl)cyclopropyl)methyl)morpholine (Int-3b). MS (ESI): m/z (M+H)⁺ 660/662.

### Step C: 4-((1-(((7-bromo-6,8-difluoro-4-(6-fluoro-8-(2-phenylpropan-2-yl)-3,8-diazabicyclol[3.2.1]octan-3-yl)quinazolin-2-yl)oxy)methyl)cyclopyrooyl)methyl)morpholine (Int-3c-1 and Int-3c-2)

The racemic 4-((1-(((7-bromo-6,8-difluoro-4-(6-fluoro-8-(2-phenylpropan-2-yl)-3,8-diazabicyclo[3.2.1]octan-3-yl)quinazolin-2-yl)oxy)methyl)cyclopropyl)methyl)morpholine (Int-3b) (67 mg, 0.10 mmol) was separated by SFC (Column P; Condition: 0.05% diethylamine in Methanol) to afford 4-((1-(((7-bromo-6,8-difluoro-4-(6-fluoro-8-(2-phenylpropan-2-yl)-3,8-diazabicyclo[3.2.1]octan-3-yl)quinazolin-2-yl)oxy)methyl)cyclopropyl)methyl)morpholine (Int-3c-1, the first eluting isomer). MS (ESI): m z (M+H)⁺ 660/662, 4-((1-(((7-bromo-6,8-difluoro-4-(6-fluoro-8-(2-phenylpropan-2-yl)-3,8-diazabicyclo[3.2.1]octan-3-yl)quinazolin-2-yl)oxy)methyl)cyclopropyl)methyl)morpholine (Int-3c-2, the second eluting isomer) was also isolated. MS (ESI): m/Z (M+H)⁺ 660/662.

### Step D: 4-((1-(((6.8-difluoro-4-(6-fluoro-8-(2-phenylpropan-2-yl)-3,8-diazabicyclo[3.2.1]octan-3-yl)-7-(3-(methoxymethoxy)naphthalen-1-yl)quinazolin-2-yl)oxy)methyl cyclopropyl)methyl)morpholine (Int-3d-1)

To a mixture of 4-((l-(((7-bromo-6,8-difluoro-4-(6-fluoro-8-(2-phenylpropan-2-yl)-3,8-diazabicyclo[3.2.1]octan-3-yl)quinazolin-2-yl)oxy)methyl)cyclopropyl)methyl)morpholine (Int-3c-1) (20 mg, 0.030 mmol) in dioxane (2.0 mL) and water (0.10 mL) were added 2-(3-(methoxymethoxy)naphthalen-1-yl)-4,4,5,5-tetramethyl-1,3,2-dioxaborolane (Int-MMM3) (14 mg, 0.045 mmol) and potassium phosphate tribasic (1.5 M in water, 0.061 mL, 0.091 mmol), followed by [1.1′-bis(di-tert-butylphosphino)ferrocene]dichloropalladium(II) (2.0 mg, 3.0 μmol). The mixture was heated to 80° C. for 2 h. After 2 hours, the mixture was allowed to cool to room temperature and then diluted with water. The mixture was extracted with EtOAc (10 mL×2). The combined organic phases were dried over anhydrous Na₂SO₄, filtered and concentrated in vacuo. The residue was purified by reverse phase prep-HPLC (MeCN/water with 0.1% TFA modifier) to afford 4-((1-(((6,8-difluoro-4-(6-fluoro-8-(2-phenylpropan-2-yl)-3,8-diazabicyclo[3.2.1]octan-3-yl)-7-(3-(methoxymethoxy)naphthalen-1-yl)quinazolin-2-yl)oxy)methyl)cyclopropyl)methyl)morpholine (Int-3d-1). MS (ESI): m/z (M+H)⁺ 768.

### Step E: 4-(6,8-difluoro-4-(6-fluoro-3,8-diazabicyclo[3.2.1]octan-3-yl)-2-((1-(morpholinomethyl)cyclopropyl)methoxy)quinazolin-7-yl)naphthalen-2-ol (Ex

To a mixture of 4-((1-(((6,8-difluoro-4-(6-fluoro-8-(2-phenylpropan-2-yl)-3,8-diazabicyclo[3.2.1]octan-3-yl)-7-(3-(methoxymethoxy)naphthalen-1-yl)quinazolin-2-yl)oxy)methyl)cyclopropyl)methyl)morpholine (Int-3d-1) (20 mg, 0.026 mmol) in toluene (2.0 mL) were added TFA (10.0 μl, 0.130 mmol) and L-cysteine hydrochloride (8.2 mg, 0.052 mmol). The mixture was stirred at 25° C. for 2 h. After 2 hours, the mixture was concentrated under reduced pressure and the resulting mixture was purified by reverse phase prep-HPLC (MeCN/water with 0.1% TFA modifier) to afford 4-(6,8-difluoro-4-(6-fluoro-3,8-diazabicyclo[3.2.1]octan-3-yl)-2-((1-(morpholinomethyl)cyclopropyl)methoxy)quinazolin-7-yl)naphthalen-2-ol (Ex. 3a). MS (ESI): m/z (M+H)⁺ 606. ¹H NMR (4(0) MHz, methanol-d₆) δ 7.83-7.63 (m, 2H). 7.43 (t, J=7.4 Hz. 1H). 7.36-7.07 (m, 4H), 5.75-5.48 (m, 1H). 4.74 (m, 1H), 4.53-4.35 (m, 5H), 4.16-3.71 (m, 8H), 3.37 (s, 2H), 3.18 (br s, 2H), 2.77 (dt, J=6.5, 16.5 Hz, 1H), 2.49-2.27 (m, 1H), 1.01-0.83 (m, 4H).

The synthesis of Ex. 3b is similar to Ex. 3a, using Int-3c-2 instead of Int-3c-1 in Step D. MS (ESI): m/z (M+H)⁺ 606. ¹H NMR (400 MHz, methanol-d₄) δ 7.79-7.63 (m, 2H), 7.43 (t, J=7.6 Hz, 1H), 7.34-7.09 (m, 4H), 5.73-5.52 (m, 1H), 4.74 (m, 1H), 4.54-4.38 (m, 5H), 4.12-3.73 (m, 8H), 3.37 (s, 2H), 3.18 (br s, 2H), 2.77 (dt, J=7.2, 16.5 Hz, 1H), 2.51-2.28 (m, 1H), 1.00-0.84 (m, 4H).

### Example 4: 4-(4-(3.8-diazabicyclo[3.2.1]octan-3-yl)-6,8-difluoro-2-((3-(hydroxymethyl)azetidin-3-yl)methoxy)quinazolin-7-yl)naphthalen-2-ol (Ex. 4)

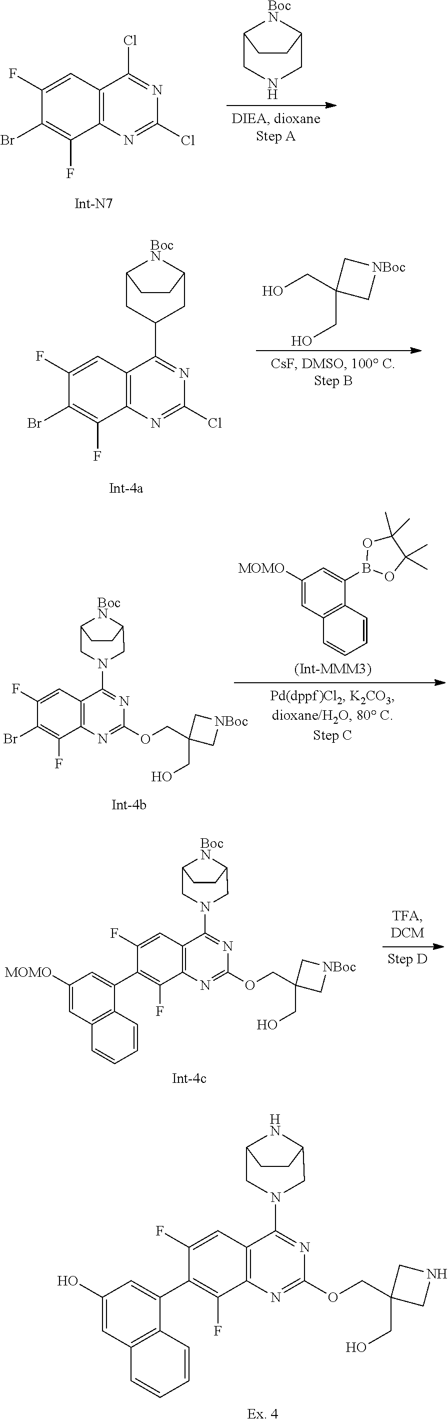

### Step A: tert-butyl 3-(7-bromo-2-chloro-6,8-difluoroquinazolin-4-yl)-3,8-diazabicyclo[3.2.1]octane-8-carboxylate (Int-4a)

To a mixture of 7-bromo-2,4-dichloro-6,8-difluoroquinazoline (Int-N7) (0.15 g, 0.48 mmol) in 1.4-dioxane (3.0 mL) were added DIEA (0.17 mL, 0.96 mmol) and terl-butyl 3.8-diazabicyclo[3.2.1]octane-8-carboxylate (0.12 g, 0.57 mmol) at 25° C. The mixture was stirred at 25° C. for 1 h and then quenched with water (3.0 mL). The mixture was extracted with EtOAc (2×6 mL). The combined organic phases were dried over Na₂SO₄, filtered and then concentrated under reduced pressure. The resulting crude product was purified by preparative TLC (SiO₂, petroleum ether: ethyl acetate=3:1) to afford tert-butyl 3-(7-bromo-2-chloro-6,8-difluoroquinazolin-4-yl)-3,8-diazabicyclo[3.2.1]octane-8-carboxylate (Int-4a). MS (ESI): m/z (M+H)⁺ 489, 491.

### Step B: tert-butyl 3-(7-bromo-2-((1-(tert-butoxycarbonyl)-3-(hydroxymethyl)azetidin-3-yl)methoxy)-6,8-difluoroquinazolin-4-yl)-3,8-diazabicyclo[3.2.1]octane-8-carboxylate (Int-4b)

To a stirred mixture of tert-butyl 3-(7-bromo-2-chloro-6,8-difluoroquinazolin-4-yl)-3,8-diazabicyclo[3.2.1]octane-8-carboxylate (Int-4a) (0.15 g, 0.31 mmol) and tert-butyl 3.3-bis(hydroxymethyl)azetidine-1-carboxylate (0.13 g, 0.61 mmol) in DMSO (5.0 mL) was added CsF (0.19 mg, 1.2 mmol) at 20° C. under a N₂ atmosphere. The mixture was heated to 100° C. for 3 h. After 3 h, the mixture was allowed to cool to room temperature and then quenched with water (20 mL). The mixture was extracted with EtOAc (8 mL×3). The combined organic layers were dried over Na₂SO₄, filtered and concentrated in vacuo. The resulting residue was purified by Prep-TLC (SiO₂, Pet, ether/EtOAc=1:2) to afford tert-butyl 3-(7-bromo-2-((1-(tert-butoxycarbonyl)-3-(hydroxymethyl)azetidin-3-yl)methoxy)-6,8-difluoroquinazolin-4-yl)-3,8-diazabicyclo[3.2.1]octane-8-carboxylate (Int-4b). MS (ESI) m/z (M+H)⁺ 670/672.

### Step C: tert-butyl 3-(2-((1-(tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-4c)

To a stirred mixture of 2-(3-(methoxymethoxy)naphthalen-1-yl)-4,4,5,5-tetramethyl-1,3,2-dioxaborolane (Int-MMM3) (14 mg, 0.045 mmol), terl-butyl 3-(7-bromo-2-((1-(tert-butoxycarbonyl)-3-(hydroxymethyl)azetidin-3-yl)methoxy)-6,8-difluoroquinazolin-4-yl)-3,8-diazabicyclo[3.2.1]octane-8-carboxylate (Int-4b) (30 mg, 0.045 mmol) in dioxane (1.0 mL) and water (0.20 mL) were added PdCl₂(dppf) (6.6 mg, 9.0 μmol) and K₂CO₃ (15 mg, 0.11 mmol) at 20° C. under a N₂ atmosphere. The mixture was heated to 80° C. for 2 h. After 2 hours, the mixture was allowed to cool to room temperature and then Directly purified by Prep-TLC (SiO₂, Pet, ether/EtOAc=1:2) to afford tert-butyl 3-(2-((1-(tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-4c). MS (ESI) m/z (M+H)⁺ 778.

### Step D: 4-(4-(3.8-diazabicyclo[3.2.1]octan-3-yl)-6,8-difluoro-2-((3-(hydroxymethyl)azetidin-3-yl)methoxy)quinazolin-7-yl)naphthalen-2-ol (Ex. 4)

To a stirred mixture of tert-butyl 3-(2-((1-(tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-4c) (15 mg, 0.019 mmol) and CH₂Cl₂ (2.0 mL) was added TFA (0.50 mL) at 20° C. and the mixture was stirred at 20° C. for 3 h. After 3 h, the mixture was concentrated under reduced pressure. The resulting residue was purified by reverse phase Prep-HPLC (MeCN/water with 0.1% TFA modifier) to afford 4-(4-(3.8-diazabicyclo[3.2.1l]octan-3-yl)-6,8-difluoro-2-((3-(hydroxymethyl)azetidin-3-yl)methoxy)quinazolin-7-yl)naphthalen-2-ol (Ex. 4). MS (ESI) m/z (M+H)⁺ 534, 1H NMR (500 MHz, methanol-d,) δ 7.78 (d, J=8.54 Hz, 11H), 7.72 (d, J=9.16 Hz. 1H), 7.45 (t, J=7.63 Hz, 1H), 7.35 (d, J=8.54 Hz, 1H), 7.31 (d, J=2.44 Hz, 1H), 7.26-7.21 (m, 1H), 7.13 (d, J=2.44 Hz, 1H), 4.71 (m, 2H), 4.56 (s, 2H), 4.26 (br s, 2H), 4.15 (s, 4H), 3.86 (dd, J=13.43, 8.54 Hz, 2H), 3.76 (s, 1H), 3.78-3.74 (m, 1H), 2.24-2.14 (m, 4H).

### Ex. 107 Synthesis of 2-(((6,8-difluoro-7-(3-hydroxynaphthalen-1-yl-4-((1S,5R)-1-methyl-3,8-diazabicyclo[3.2.1]octan-3-yl)quinazolin-2-yl)oxy)methyl)-2-(dimethylamino)methyl)cyclopropane-1-carboxylc acid (Ex. 107a -peak 1 and Ex. 107—Peak 2)

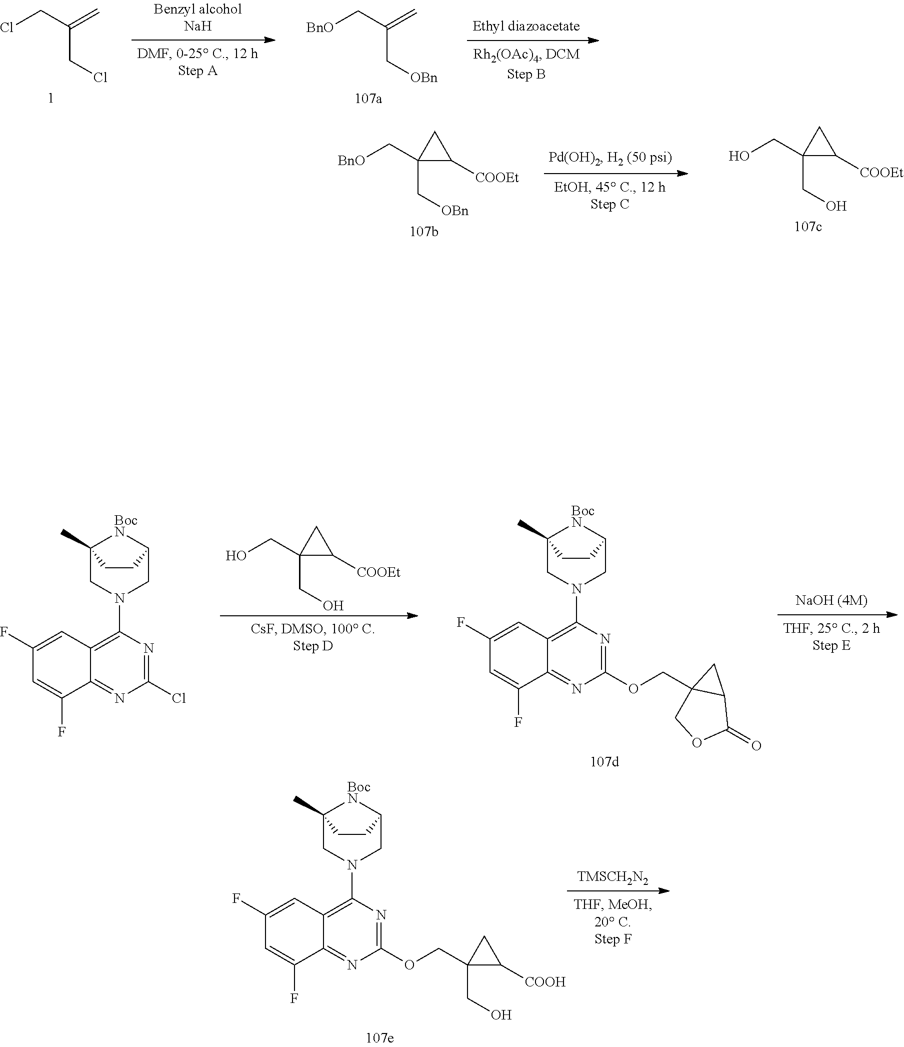

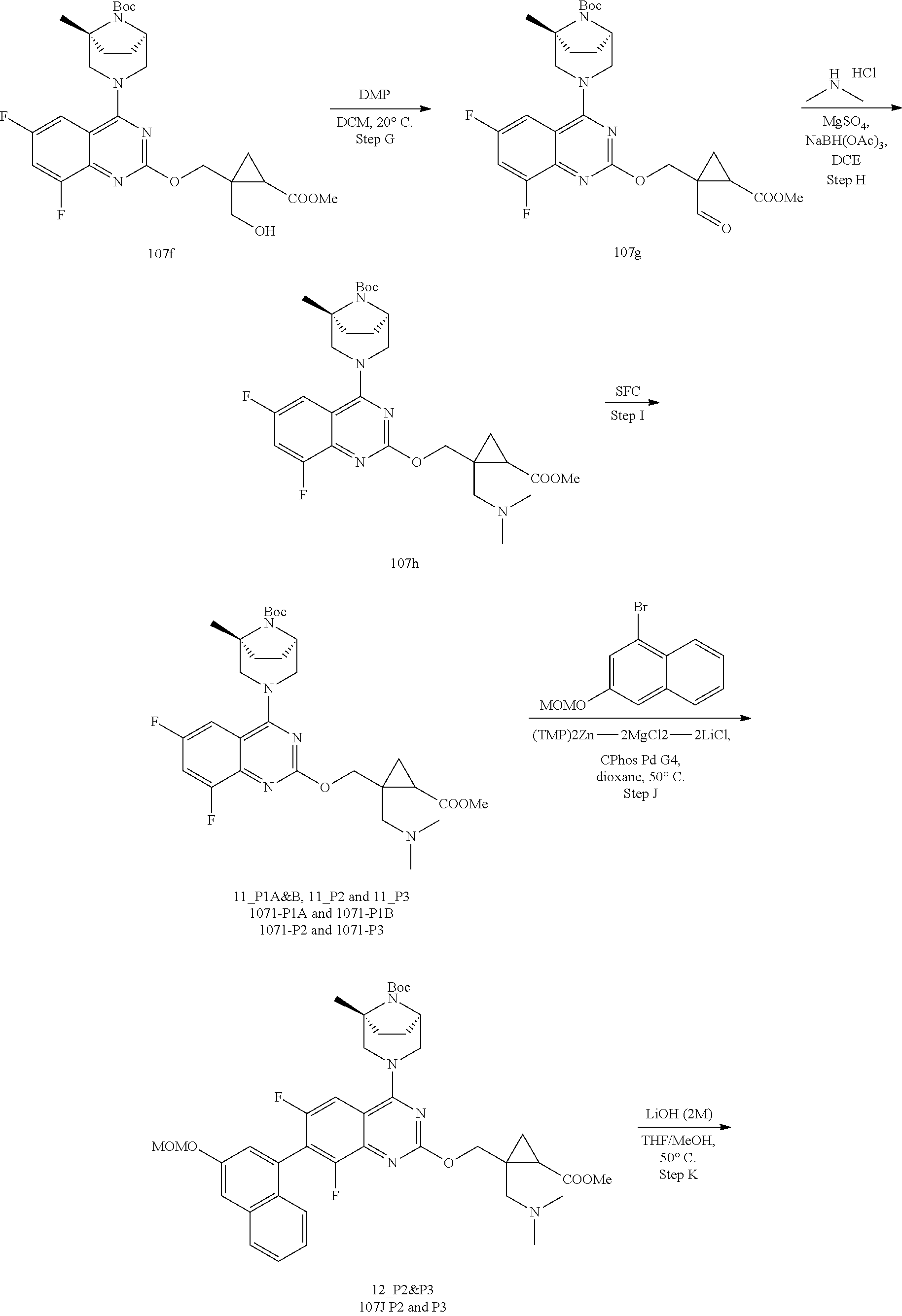

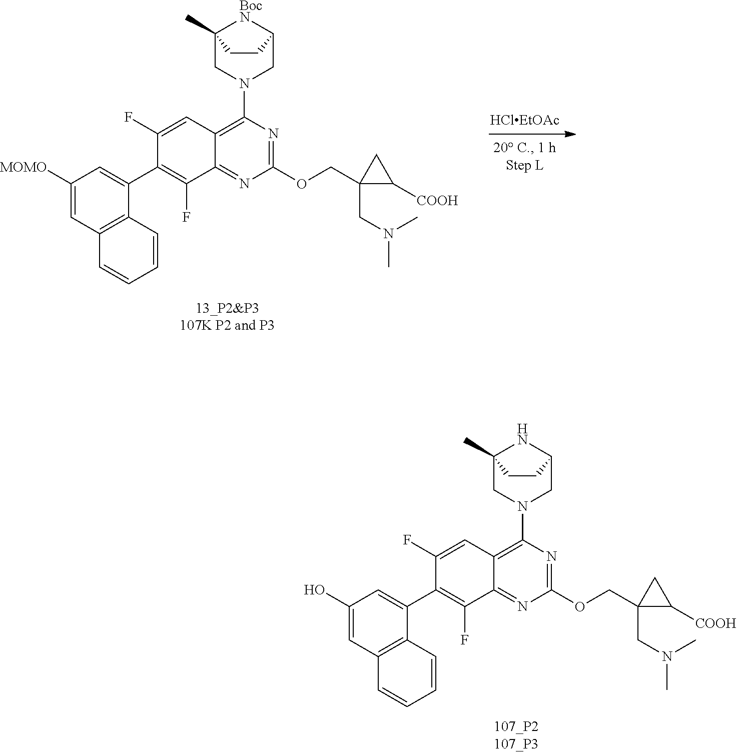

### Step A: (((2-methylenepropane-1,3-diyl)bis(oxy))bis(methylene))dibenzene (Int 107a)

dropwise and the reaction mixture was stirred for another 1 h at 0° C. Then 3-chloro-2-(chloromethyl)prop-1-ene (45.0 g, 360 mmol) was added dropwise at 0° C., and the reaction mixture was warmed to 25° C. and stirred 12 h. TLC (SiO₂, Pet, ether/EtOAc=10:1, v/v) was carried out. The reaction mixture was poured into ice water (1.5 L). The aqueous layer was extracted with EtOAc (250 mL×2). The combined organic layers were washed with brine (250 mL), dried over MgSO₄ and filtered. The filtrate was concentrated under reduced pressure to give the residue which was purified by flash silica gel chromatography 120 g silica column, Eluent of 0-16% EtOAc/Pet, ether, dry loaded to give (((2-methylenepropane-1,3-diyl)bis(oxy))bis(methylene))dibenzene. ¹H NMR (400 MHz, CDCl₃) δ 7.27-7.39 (m, 1OH), 5.28 (s, 2H), 4.53 (s, 4H), 4.09 (s, 4H).

### Step B: Ethyl 2,2-bis((benzyloxy)methyl)cyclopropane-1-carboxylate (Int-107b)

To a solution of (((2-methylenepropane-1,3-diyl)bis(oxy))bis(methylene))dibenzene (Int-107a) (10.0 g, 37.3 mmol) and rhodium(II) acetate (0.824 g, 3.73 mmol) in DCM (100 mL) was added ethyl diazoacetate (5.9 mL, 55.9 mmol) dropwise over 20 min at 25° C. The reaction mixture was stirred at 25° C. for another 30 min. The reaction mixture was diluted with DCM (100 mL) and quenched with AcOH (1 mL). The reaction mixture was filtered and the filtrate was concentrated in vacuum to give the residue which was purified by flash silica gel chromatography 80 g column, Eluent of 0-27% EtOAc/Pet, ether gradient, dry loaded to give ethyl 2,2-bis((benzyloxy)methyl) cyclopropane-1-carboxylate. ¹H NMR (400 MHz, CDCl₃) δ 7.29-7.39 (m, 1OH), 4.53 (d, J=1.96 Hz, 2H), 4.43-4.49 (m, 2H), 4.26-4.31 (m, 1H), 4.14 (d, J=7.09 Hz, 1H), 3.92 (d, J=10.03 Hz, 1H), 3.76 (d, J=9.78 Hz, 1H), 3.61 (d, J=9.78 Hz, 1H), 3.33 (d, J=9.78 Hz, 1H), 1.80 (dd, J=5.75, 7.95 Hz, 1H), 1.30 (d, J=5.62 Hz, 1H), 1.26 (t, J=7.21 Hz, 3H), 1.11-1.18 (m, 1H).

### Step C: Ethyl 2,2-bis(hydroxymethyl)cyclopropane-1-carboxylate (Int-107c)

To a solution of ethyl 2,2-bis((benzyloxy)methyl)cyclopropane-1-carboxylate (Int-107b) (9.00 g, 25.4 mmol) in EtOH (160 mL) was added palladium hydroxide on carbon (0.891 g, 1.27 mmol) (20% on active carbon). The reaction mixture was degassed and backfilled with H₂ for 3 times. The reaction mixture was stirred at 20° C. for 12 h under H₂ (50 psi). The reaction mixture was filtered and the filtrate was concentrated under reduced pressure to give the residue which was purified by flash silica gel chromatography 40 g column, Eluent of 0-19% EtOAc/Pet, ether dry loaded, to give the mixture of the starting material and mono-Bn intermediate. This mixture was dissolved in EtOH (50 mL) and palladium hydroxide on carbon (0.872 g, 1.24 mmol) (20% on active carbon) was added. The reaction mixture was degassed and backfilled with H₂ for 3 times. The reaction mixture was stirred at 45° C. for 12 h under H₂ (50 psi). ¹H NMR showed all the starting material was consumed completely. The reaction mixture was filtered and the filtrate was concentrated under reduced pressure to give ethyl 2,2-bis(hydroxymethyl)cyclopropane-1-carboxylate. The crude product was used to the next step without further purification. ¹H NMR (400 MHz, CDCl₃) δ 4.24-4.43 (m, 1H), 4.11-4.18 (m, 2H), 3.78-3.91 (m, 1H), 3.46-3.74 (m, 2H), 1.74-2.07 (m, 1H), 1.21-1,30 (m, 5H).

### Step D: Tert-butyl (1S,5R)-3-(6,8-difluoro-2-((4-oxo-3-oxabicyclo[3.1.0]hexan-1-yl)methoxy)quinazolin-4-yl)-1-methyl-3,8-diazabicyclo[3.2.1]octane-8-carboxylate (Int-107d)

To a solution of tert-butyl (1S,5R)-3-(2-chloro-6,8-difluoroquinazolin-4-yl)-1-methyl-3,8-diazabicyclo[3.2.1]octane-8-carboxylate (Int-D3) (1,30 g, 3.06 mmol) and ethyl 2,2-bis(hydroxymethyl)cyclopropane-1-carboxylate (Int-107c) (1.60 g, 9.18 mmol) in DMSO (20 mL) was added CsF (1.63 g, 10.7 mmol). The reaction mixture was stirred at 100° C. for 3 h. The reaction was monitored by LC. The reaction mixture was quenched with H₂O (50 mL) and EtOAc (15 mL). The aqueous layer was separated and extracted with EtOAc (15 mL×2). The combined organic layers were dried over MgSO₄ and filtered. The filtrate was concentrated under reduced pressure to give the residue which was purified by flash silica gel chromatography 12 g silica column, Eluent of 0-26% EtOAc/Pet, ether gradient, dry loaded, to give tert-butyl (1S,5R)-3-(6,8-difluoro-2-((4-oxo-3-oxabicyclo[3.1.0]hexan-1-yl)methoxy)quinazolin-4-yl)-1-methyl-3,8-diazabicyclo[3.2.1]octane-8-carboxylate. MS (ESI): m/z (M+H)⁺ 517. ¹H NMR (400 MHz, CDCl₃) δ 7.30 (d, J=9.54 Hz, 1H), 7.18-7.26 (m, 1H), 4.70 (dd. J=4.40, 12.23 Hz, 1H), 4.60 (d, J=9.29 Hz, 1H), 4.51-4.58 (m, 1H), 4.43 (d, J=6.11 Hz, 1H), 4.37 (d, J=9.29 Hz, 1H), 4.25 (d, J=12.72 Hz, 1H), 4.10-4.14 (m, 1H), 3.52 (d, J=12.47 Hz, 2H), 2.24 (dd, J=3.30, 9.17 Hz, 1H), 1.94-2.03 (m. 2H), 1.78-1.86 (m, 1H), 1.68-1.75 (m, 1H), 1.61 (s, 3H), 1.52 (s, 9H), 1.25-1.29 (m, 1H), 1.16-1.22 (m, 1H).

### Step E: 2-(((4-((1S,5R)-8-(tert-butoxycarbonyl)₁-methyl-3,8-diazabicyclo[3.2.1]octan-3-yl)-6,8-difluoroquinazolin-2-yl)oxy)methyl)-2-(hydroxymethyl)cyclopropane-1-carboxylc acid (Int-107e)

To a solution of tert-butyl (1S,5R)-3-(6,8-difluoro-2-((4-oxo-3-oxabicyclo[3.1.0]hexan-1-yl)methoxy)quinazolin-4-yl)-1-methyl-3,8-diazabicyclo[3.2.1]octane-8-carboxylate (Int-107d) (920 mg, 1.78 mmol) in THF (10 mL) was added aqueous NaOH (10 mL, 40.0 mmol) (4 M). The reaction mixture was stirred at 20° C. for 2 h. The reaction was monitored by LC. The reaction mixture was diluted with EtOAc (20 mL) and H₂O (15 mL). The mixture was acidified with aqueous HCl (1 M) until pH 7 at 0 C. The aqueous layer was separated and extracted with EtOAc (20 mL×3). The combined organic layers were dried over MgSO₄ and filtered. The filtrate was concentrated under reduced pressure to give 2-(((4-((1S,5R)-8-(tert-butoxycarbonyl)-1-methyl-3,8-diazabicyclo[3.2.1]octan-3-yl)-6,8-difluoroquinazolin-2-yl)oxy)methyl)-2-(hydroxymethyl)cyclopropane-1-carboxylc acid. The crude product was used to the next step without further purification. MS (ESI): m/z (M+H)⁺ 535. ¹H NMR (4(0) MHz, CDCl₃) δ 7.30 (d, J=9.29 Hz, 1H), 7.19-7.26 (m, 1H), 4.884.96 (m, 1H), 4.43 (d, J=6.11 Hz, 1H), 4.25-4.34 (m, 1H), 4.134.19 (m, 1H). 4.04 (dd, J=2.20, 11.98 Hz, 1H), 3.96 (d, J=12.72 Hz. 1H), 3.76-3.83 (m, 1H), 3.53 (t, J=11.25 Hz, 2H), 1.97 (d, J=7.58 Hz, 2H), 1.79-1.90 (m, 2H), 1.67-1.73 (m, 1H), 1.60 (s, 3H), 1.52 (s, 9H), 1.36-1.43 (m, 1H), 1.27-1,30 (m, 1H).

### Step F: Tert-butyl (1S,5R)-3-6,8-difluoro-2-((1-(hydroxymethyl)-2-(methoxycarbonyl)cyclopropyl)methoxy)quinazolin-4-yl)-1-methyl-3,8-diazabicyclo[3.2.1]octane-8-carboxylate (Int-107f)

To a solution of 2-(((4-((1S,5R)-8-(tert-butoxycarbonyl)-1-methyl-3,8-diazabicyclo[3.2.1]octan-3-yl)-6,8-difluoroquinazolin-2-yl)oxy)methyl)-2-(hydroxymethyl)cyclopropane-1-carboxylc acid (Int-107e) (940 mg, 1.76 mmol) in THF (8.0 mL) and MeOH (1.6 mL) was added (trimethylsilyl)diazomethane (1.76 mL, 3.52 mmol) (2 M in Hexane). The reaction mixture was stirred at 20° C. for 30 min. The reaction was monitored by LC. The reaction was quenched with AcOH (0.3 mL), and concentrated under reduced pressure to give tert-butyl (I S,5R)-3-(6,8-difluoro-2-((1-(hydroxymethyl)-2-(methoxycarbonyl)cyclopropyl)methoxy)quinazolin-4-yl)-1-methyl-3,8-diazabicyclo[3.2.1]octane-8-carboxylate. The crude product was used to the next step without further workup. MS (EST): m/z (M+H)⁺ 549.

### Step G: Tert-butyl (1S,5R)-3-(6,8-difluoro-2-((1-formyl-2-(methoxycarbonyl)cyclopropyl)methoxy)quinazolin-4-yl)-1-methyl-3,8-diazabicyclo[3.2.1]octane-8-carboxylate (Int-107g)

To a solution of tert-butyl (1S,5R)-3-(6,8-difluoro-2-((1-(hydroxymethyl)-2-(methoxycarbonyl)cyclopropyl)methoxy)quinazolin-4-yl)-1-methyl-3,8-diazabicyclo[3.2.1]octane-8-carboxylate (Int-107f) (1.00 g, 1.76 mmol) in DCM (15 mL) was added DMP (1.12 g, 2.64 mmol). The reaction mixture was stirred at 20° C. for 1 hr. The reaction was monitored by LC. The reaction mixture was concentrated under reduced pressure to give the residue which was purified by flash silica gel chromatography 12 g column, Eluent of 0˜4% MeOH/DCM gradient, dry loaded, to give tert-butyl (1S,5R)-3-(6,8-difluoro-2-((1-formyl-2-(methoxycarbonyl)cyclopropyl)methoxy)quinazolin-4-yl)-1-methyl-3,8-diazabicyclo[3.2.1]octane-8-carboxylate. MS (ESI), m/z (M+H)⁺ 547. ¹H NMR (400 MHz, CDCl₃) δ 9.49 (s, 1H), 7.17-7.26 (m, 2H), 4.75-4.79 (m, 1H), 4.56 (d, J=11.49 Hz, 1H), 4.42 (d, J=6.11 Hz, 1H), 4.26 (d, J=12.72 Hz, 1H), 4.09 (d, J=12.72 Hz, 1H), 3.71-3.77 (m, 4H), 3.45-3.57 (m, 3H), 2.51-2.55 (m, 1H), 2.11-2.15 (m, 1H), 1.94-1.99 (m, 2H), 1.80-1.87 (m, 2H), 1.63-1.73 (m, 2H), 1.52 (s, 9H).

### Step H: Tert-butyl (1S,5R)-3-(2-((1-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate (Int-107h)

To a solution of tert-butyl (1S,5R)-3-(6,8-difluoro-2-((1-formyl-2-(methoxycarbonyl)cyclopropyl)methoxy)quinazolin-4-yl)-1-methyl-3,8-diazabicyclo[3.2.1]octane-8-carboxylate (Int-107g) (500 mg, 0.915 mmol), MgSO₄ (110 mg, 0.915 mmol) in DCE (10 mL) was added dimethylamine, HCl (295 mg, 3.66 mmol). The reaction mixture was stirred at 50° C. for 30 min. The reaction mixture was cooled to 20° C. and sodium triacetoxyborohydride (194 mg, 0.915 mmol) was added. The reaction mixture was stirred at 20° C. for another 12 h. The reaction was monitored by LC. The reaction mixture was diluted with DCM (20 mL) and filtered. The filtrate was concentrated under reduced pressure to give the crude product. The crude product was diluted with EtOAc (15 mL) and NaHCO₃(15 mL) (aq.). The mixture was separated and the aqueous layer was extracted with EtOAc (15 mL×3). The combined organic layers were washed with brine (15 mL), dried over MgSO₄ and filtered. The filtrate was concentrated under reduced pressure to give the residue which was purified by flash silica gel chromatography 12 g column, Eluent of 0 ˜ 4% MeOH/DCM gradient, dry loaded, to give tert-butyl (1S,5R)-3-(2-((1-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate. MS (ESI): m/z (M+H)⁺ 576.

### Step 1: Tert-butyl (1S,5R)-3-(2-((1-((dimethylamino)methyl)-2-(methoxy carbonyl)cyclopropyl)methoxy)-6,8-difluoroquinazolin-4-yl)-1-methyl-3,8-diazabicyclo[3.2.1]octane-8-carboxylate (Int-1071 PIA and P1B) and (Int-1071 P2 and Int-1071 P3)

The racemic mixture of tert-butyl (I S,5R)-3-(2-((1-((dimethylamino)methyl)-2-(methoxycarbonyl)cyclopropyl)methyl)-6,8-difluoroquinazolin-4-yl)-1-methyl-3,8-diazabicyclo[3.2.1]octane-8-carboxylate (620 mg, 1.077 mmol) was separated by preparative SFC (Column REG1S (R, R) WHELK-O1 (250 mm*25 mm, 10 um), Condition 0.1% NH₃H₂O IPA to afford tert-butyl (1S,5R)-3-(2-((1-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate (1071_P1A and 1071 P1B, the first eluting isomer from SFC), tert-butyl (1S,5R)-3-(2-((1-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate (1071_P2, the second eluting isomer from SFC) and tert-butyl (1S,5R)-3-(2-((I-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate (1071_P3, the third eluting isomer from SFC). MS (ESI): m/z (M+H)⁺ 576.

### Step J: Tert-butyl (1S,5R)-3-(2-((I-(dimethylamino)methyl)-2-(methoxycarbonyl)cyclopropyl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (107J_P2)

To a solution of tert-butyl (1S,5R)-3-(2-((1-((dimethylamino)methyl)-2-(methoxycarbonyl)cyclopropyl)methoxy)-6,8-difluoroquinazolin-4-yl)-1-methyl-3,8-diazabicyclo[3.2.1]octane-8-carboxylate (Int1071_P2) (100 mg, 0.174 mmol) in dry THF (1.0 mL) was added (TMP)₂Zn·2MgCl₂·2LiCl (1.6 mL, 0.521 mmol) (0.33 M in THF) dropwise at 25° C. under N₂ atmosphere. After addition, the reaction mixture was stirred at 50° C. for 1 h. Then one drop reaction mixture was quenched with 12. The reaction was monitored by LC. A solution of CPhos Pd G3 (14.01 mg, 0.017 mmol) and 1-bromo-3-(methoxymethoxy)naphthalene (60.3 mg, 0.226 mmol) in THF (0.5 mL) was added to the above solution, after addition, the reaction was stirred at 50° C. for 12 h. The reaction was monitored by LC. The reaction mixture was diluted with EtOAc (20 mL) and NaHCO₃(5 mL) (aq.) was added. The mixture was filtered, and the organic layer was obtained by separated. The organic layer was washed with brine (5 mL), dried over MgSO₄ and filtered. The filtrate was concentrated under reduced pressure to give the residue which was purified by flash silica gel chromatography 4 g column, Eluent of 0 ˜ 4% MeOH/DCM gradient, dry loaded, to give tert-butyl (1S,5R)-3-(2-((1-((dimethylamino)methyl)-2-(methoxycarbonyl)cyclopropyl)methoxy)-6,8-difluoro-7-(3-(methoxymethoxy) naphthalen-1-yl)quinazolin-4-yl)-1-methyl-3,8-diazabicyclo[3.2.1]octane-8-carboxylate. MS (ESI): m/z (M+H)⁺ 762.

### Step K: 2-(((4-((1S,5R)-8-(tert-butoxycarbonyl)--methyl-3,8-diazabicyclo[3.2.1]octan-3-yl)-6,8-difluoro-7-(3-(methoxymethoxy)naphthalen-1-yl)quinazolin-2-yloxy)methyl)-2-((dimethylamino)methyl cyclopropane-1-carboxylc acid (107K_P2)

To a solution of tert-butyl (1S,5R)-3-(2-((1-((dimethylamino)methyl)-2-(methoxycarbonyl)cyclopropyl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (107J_P2)(50.0 mg, 0.066 mmol) in MeOH (0.3 mL) and THF (0.3 mL) was added lithium hydroxide monohydrate (0.33 mL, 0.656 mmol) (2 M in H₂O). The reaction mixture was stirred at 50° C. for 1 h. The reaction was monitored by LC. The reaction mixture was concentrated under reduced pressure to remove most solvent. The residue was diluted with EtOAc (3 mL) and H₂O (1 mL). The reaction mixture was acidified with aqueous HCl (1 M) until pH 7 at 0 C. The aqueous layer was separated and extracted with EtOAc (5 mL×3). The combined organic layers were dried over Na₂SO₄ and filtered. The filtrate was concentrated under reduced pressure to give 2-(((4-((1S,5R)-8-(tert-butoxycarbonyl)-1-methyl-3,8-diazabicyclo[3.2.1]octan-3-yl)-6,8-difluoro-7-(3-(methoxymethoxy)naphthalen-1-yl)quinazolin-2-yl)oxy)methyl)-2-((dimethylamino)methyl)cyclopropane-1-carboxylc acid. The crude product was used to the next step without further purification. MS (ESI): m/z (M+H)⁺ 748.

### Step L: 2-(((6,8-difluoro-7-(3-hydroxynaphthalen-1-yl)-4-((1S,5R)-1-methyl-3,8-diazabicyclo[3.2.1]octan-3-yl)quinazolin-2-yl)oxy)methyl)-2-((dimethylamino)methyl)cyclopropane-1-carboxylc acid (Ex. 107 P2 001)

A solution of 2-(((4-((1S,5R)-8-(tert-butoxycarbonyl)-1-methyl-3,8-diazabicyclo[3.2.1]octan-3-yl)-6,8-difluoro-7-(3-(methoxymethoxy)naphthalen-1-yl)quinazolin-2-yl)oxy)methyl)-2-((dimethylamino)methyl)cyclopropane-1-carboxylc acid (107K_P2) (49.0 mg, 0.066 mmol) in HCl/EtOAc (4 M in EtOAc) (1.0 mL) was stirred at 20° C. for 1 h. The reaction was monitored by LC. The reaction mixture was concentrated under reduced pressure (25° C.) to give the residue which was purified by Prep-HPLC (Column Waters Xbridge BEH C18 100×25 mm×Sum, Condition water (0.225% FA)—ACN to give 2-(((6,8-difluoro-7-(3-hydroxynaphthalen-1-yl)-4-((1S,5R)-1-methyl-3,8-diazabicyclo[3.2.1]octan-3-yl)quinazolin-2-yl)oxy)methyl)-2-((dimethylamino)methyl)cyclopropane-1-carboxylc acid. MS (ESI): m/z (M+H)⁺ 604, 107_P2_001: ¹H NMR (4(0) MHz, MeOD) δ 7.77 (d, J=8.31 Hz, 1H), 7.72 (d, J=9.78 Hz, 1H), 7.43 (t, J=7.58 Hz, 1H), 7.35 (d, J=8.31 Hz, 1H), 7.29 (d, J=1.96 Hz, 1H), 7.19-7.25 (m, 1H), 7.12 (d, J=2.45 Hz, 1H), 4.63-4.70 (m, 1H), 4.57 (dd, J=9.17, 13.57 Hz, 1H), 4.45-4.53 (m, 1H), 4.36 (dd, J=9.41, 11.86 Hz. 1H), 4.12-4.20 (m, 1H), 3.73-3.82 (m, 1H), 3.61-3.71 (m, 3H), 2.93 (s, 6H), 2.10-2.27 (m, 3H), 1.98 (t, J=6.85 Hz, 11H), 1.84 (d, J=3.91 Hz, 1H), 1.57 (s, 3H), 1.29-1,35 (m, 2H). The procedure of 107_P3_001 was similar as 107_P2_001 using Int-107I_P3 (11_P3), and the spectrum was showed below: 107_P3_001: ¹H NMR (400 MHz, MeOD) δ 7.77 (d, J=8.22 Hz, 1H), 7.71 (d, J=9.00 Hz, 1H), 7.43 (t, J=7.43 Hz, 1H), 7.33-7.38 (m, 1H), 7.29 (d, J=1.96 Hz. 1H), 7.20-7.25 (m, 1H), 7.12 (s, 1H), 4.59-4.66 (m, 1H), 4.47-4.55 (m, 2H), 4.32 (t, J=11.35 Hz, 1H), 3.99 (d, J=5.48 Hz, 1H), 3.66-3.73 (m, 3H), 3.51-3.59 (m, 1H), 2.92 (d, J=1.96 Hz, 6H), 2.07-2.17 (m, 2H), 2.03 (d, J=10.56 Hz, 1H), 1.92-1.97 (m, 1H), 1.68-1.77 (m, 1H), 1.49 (s, 3H), 1.29 (d, J=7.83 Hz, 2H).

### Example 5: 4-(4-(3.8-diazabicyclo[3.2.1]octan-3-yl)-6-chloro-8-fluoro-2-(((S)-1-methylpyrrolidin-2-yl)methoxy)quinazolin-7-yl)naphthalen-2-ol (Ex. 5)

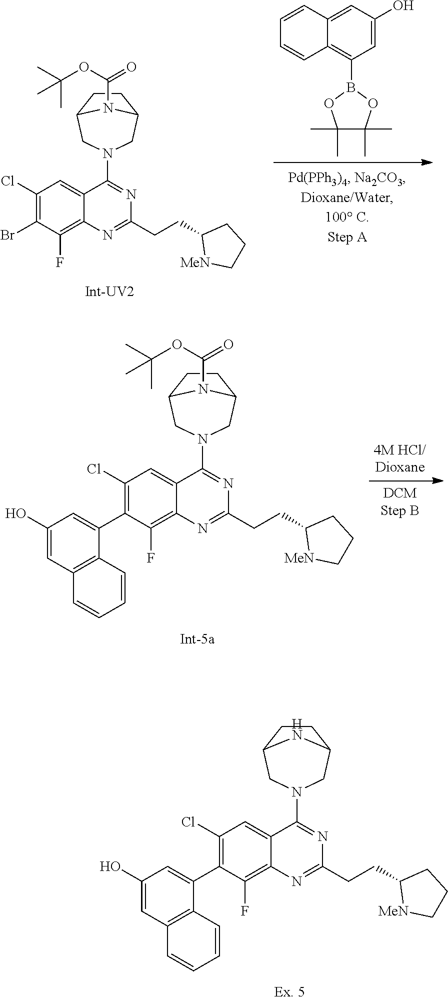 [SMILES: CN1CCC[C@H]1CCc1nc(N2CC3CCC(C2)N3C(=O)OC(C)(C)C)c2cc(Cl)c(Br)c(F)c2n1 ; CC1(C)OB(c2cc(O)cc3ccccc23)OC1(C)C ; CN1CCC[C@H]1CCc1nc(N2CC3CCC(C2)N3C(=O)OC(C)(C)C)c2cc(Cl)c(-c3cc(O)cc4ccccc34)c(F)c2n1 ; CN1CCC[C@H]1CCc1nc(N2CC3CCC(C2)N3)c2cc(Cl)c(-c3cc(O)cc4ccccc34)c(F)c2n1]

### Step A: tert-butyl 346-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octane-8-carboxylate (Int-5a)

To a mixture of tert-butyl 3-(7-bromo-6-chloro-8-fluoro-2-(((S)-1-methylpyrrolidin-2-yl)methoxy)quinazolin-4-yl)-3,8-diazabicyclo[3.2.1]octane-8-carboxylate (Int-UV2) (50 mg, 0.085 mmol), 4-(4,4,5,5-tetramethyl-1,3,2-dioxaborolan-2-yl)naphthalen-2-ol (92 mg, 0.34 mmol), tetrakis(triphenylphosphine)-palladium(0) (19 mg, 0.017 mmol) and sodium carbonate (27 mg, 0.26 mmol) were added 1.4-dioxane (0.90 mL) and water (0.20 mL). The mixture was heated to 100° C. for 16 h. After 16 h, the mixture was allowed to cool to room temperature, diluted with DCM and washed with water. The organic layer was concentrated under reduced pressure. The crude tert-butyl 3-(6-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octane-8-carboxylate (Int-Sa) was used in subsequent transformations without further purification. MS (ESI): m/z (M+H)⁺ 648.

### Step B: 4-(4-(3.8-diazabicyclo[3.2.1]octan-3-yl)-6-chloro-8-fluoro-2-(((S)-1-methylpyrrolidin-2-yl)methoxy)quinazolin-7-yl)naphthalen-2-ol (Ex. 5)

To a mixture of tert-butyl 3-(6-chloro-8-fluoro-7-(3-hydroxynaphthalen-1-yl)-2-(((S)-1-methylpyrrolidin-2-yl)methoxy)quinazolin-4-yl)-3,8-diazabicyclo[3.2.1]octane-8-carboxylate (Int-Sa) (55 mg, 0.085 mmol) in DCM (0.40 mL) was added HCl (4.0 M in dioxane, 0.10 mL, 0.40 mmol). The reaction mixture was stirred at room temperature for 2 h. Ether was then added to the resulting mixture. The resulting solid was filtered and washed with ether. The solid was purified by reverse phase prep-HPLC (MeCN/water with 0.1% ammonium hydroxide modifier) to afford 4-(443,8-diazabicyclo[3.2.1]octan-3-yl)-6-chloro-8-fluoro-2-(((S)-1-methylpyrrolidin-2-yl)methoxy)quinazolin-7-yl)naphthalen-2-ol (Ex. 5). MS (ESI): m/z (M+H)⁺ 548. ¹H NMR (600 MHz, DMSO-d₆) δ 10.01 (s, 1H), 7.94 (s, 1H), 7.81 (d, J=8.3 Hz, 1H), 7.44 (dt, J=8.1, 3.9 Hz, 1H), 7.28 (d, J=2,3 Hz, 1H), 7.24-7.21 (m, 2H), 7.06 (d, J=2,3 Hz, 1H), 4.42-4.29 (m, 3H), 4.15 (dt, J=10.8, 6.8 Hz, 1H), 3.58-3.48 (m, 4H), 2.95 (dt, J=8.8, 4.1 Hz, 1H), 2.62-2.54 (m, 1H), 2.35 (d, J=1.4 Hz, 3H), 2.17 (q, J=8.8 Hz, 1H), 1.94 (s, 1H), 1.66 (d, J=8.4 Hz, 4H).

### Example 6a: 4-(2-((1-((dimethylamino)methyl)cyclopropyl)methoxy)-6,8-difluoro-4-((1S,3S,4S)-3-vinyl-2,5-diazabicyclo[2.2.2]octan-2-yl)quinazolin-7-yl)naphthalen-2-ol (Ex. 6a)

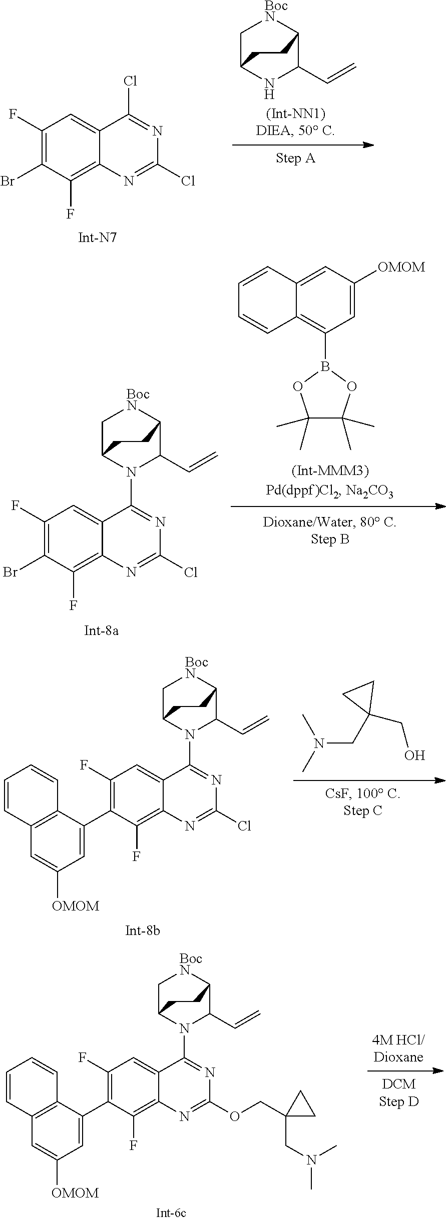

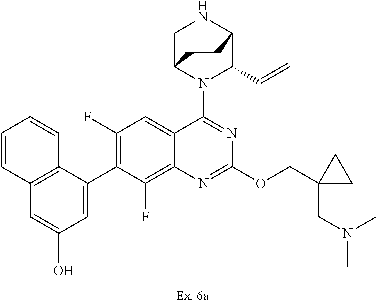 [SMILES: C=C[C@H]1[C@@H]2CC[C@@H](CN2)N1c1nc(OCC2(CN(C)C)CC2)nc2c(F)c(-c3cc(O)cc4ccccc34)c(F)cc12]

### Step A: tert-butyl (1S,4S)-547-bromo-2-chloro-6,8-difluoroquinazolin-4-yl)-6-vinyl-2,5-diazabicyclo[2.2.2]octane-2-carboxylate (Int-6a)

To a mixture of 7-bromo-2,4-dichloro-6,8-difluoroquinazoline (Int-N₇) (0.20 g, 0.64 mmol) and DIEA (0.56 mL, 3.2 mmol) in dioxane (3.3 mL) was added tert-butyl (1S,4S)-6-vinyl-2,5-diazabicyclo[2.2.2]octane-2-carboxylate (tran:cis=2.5:1) (Int-NN₁) (0.16 g, 0.67 mmol). The mixture was heated to 50° C. for 20 minutes. After 20 minutes, the mixture was allowed to cool to rt and partitioned between water and DCM. The organic layer was separated, dried over magnesium sulfate, filtered and concentrated under reduced pressure. The crude product was purified by column chromatography on silica (0 to 30% EtOAc gradient in hexanes) to afford tert-butyl (1S,4S)-5-(7-bromo-2-chloro-6,8-difluoroquinazolin-4-yl)-6-vinyl-2,5-diazabicyclo[2.2.2]octane-2-carboxylate (Int-6a). MS (ESI): m/z (M+H)⁺ 515, 517.

### Step B: tert-butyl (1S,4S)-5-(2-chloro-6,8-difluoro-7-(3-(methoxymethoxy)naphthalen-1-yl)quinazolin-4-yl)-6-vinyl-2,5-diazabicyclo[2.2.2]octane-2-carboxylate (Int-6b)

A mixture of tert-butyl (1S,4S)-5-(7-bromo-2-chloro-6,8-difluoroquinazolin-4-yl)-6-vinyl-2,5-diazabicyclo[2.2.2]octane-2-carboxylate (Int-6a) (73 mg, 0.14 mmol), [1.1′-bis(diphenylphosphino)ferrocene]dichloropalladium(II) (20 mg, 0.028 mmol), sodium carbonate (37 mg, 0.35 mmol) and 2-(3-(methoxymethoxy)naphthalen-1-yl)-4,4,5,5-tetramethyl-1,3,2-dioxaborolane (Int-MMM3) (0.14 g, 0.56 mmol) in 1,4-dioxane (760 μl) and water (190 μl) was heated to 80° C. for 2 h under a nitrogen atmosphere. The resulting mixture was allowed to cool to rt and partitioned between water and DCM. The organic layer was separated and concentrated under reduced pressure. The crude product was purified by column chromatography on silica (0 to 50% ethyl acetate gradient in hexanes) to afford tert-butyl (1S,4S)-5-(2-chloro-6,8-difluoro-7-(3-(methoxymethoxy)naphthalen-1-yl)quinazolin-4-yl)-6-vinyl-2,5-diazabicyclo[2.2.2]octane-2-carboxylate (Int-6b). MS (ESI): m/z (M+H)⁺ 623.

### Step C: tert-butyl (1S,4S)-5-(2-((1-((dimethylamino)methyl)cyclopropyl)methoxy)-6,8-difluoro-7-(3-(methoxymethoxy)naphthalen-1-yl)quinazolin-4-yl)-6-vinyl-2,5-diazabicyclo[2.2.2]octane-2-carboxylate (Int-6c

To a mixture of (1-((dimethylamino)methyl)cyclopropyl)methanol (20 mg, 0.16 mmol) and tert-butyl (1S,4S)-5-(2-chloro-6,8-difluoro-7-(3-(methoxymethoxy)naphthalen-1-yl)quinazolin-4-yl)-6-vinyl-2,5-diazabicyclo[2.2.2]octane-2-carboxylate (Int-6b) (39 mg, 0.063 mmol) in DMSO (630 μl) was added cesium fluoride (38 mg, 0.25 mmol). The reaction mixture was heated to 100° C. for 1.5 h. The resulting mixture was then allowed to cool to room temperature and partitioned between water and DCM. The organic laver was separated and concentrated under reduced pressure to afford tert-butyl (1S,4S)-5-(2-((l-((dimethylamino)methyl)cyclopropyl)methoxy)-6,8-difluoro-7-(3-(methoxymethoxy)naphthalen-1-yl)quinazolin-4-yl-6-vinyl-2,5-diazabicyclo[2.2.2]octane-2-carboxylate (Int-6c) which was used in subsequent transformations without further purification. MS (ESI): m/z (M+H)⁺ 716.

### Step D: 4-(2-((1-((dimethylamino)methyl)cyclopropyl)methoxy)-6,8-difluoro-4-((1S,3S,4S)-3-vinyl-2,5-diazabicyclo[2.2.2]octan-2-yl)quinazolin-7-yl)naphthalen-2-ol (Ex. 6a)

DCM (0.60 mL) was added to a 20 mL scintillation vial containing tert-butyl (1S,4S)-5-(2-((1-((dimethylamino)methyl)cyclopropyl)methoxy)-6,8-difluoro-7-(3-(methoxymethoxy)naphthalen-1-yl)quinazolin-4-yl)-6-vinyl-2,5-diazabicyclo[2.2.2]octane-2-carboxylate (Int-6c) (44 mg, 0.061 mmol). HCl (4.0 M in dioxane, 0.15 mL. 0.60 mmol) was added. The reaction mixture was stirred at room temperature for 2 h. Ether was then added to the resulting mixture. The resulting solids were filtered and washed with ether. The solids were then purified by reverse phase HPLC (MeCN/water with 0.1% ammonium hydroxide modifier) to afford 4-(2-((1-((dimethylamino)methyl)cyclopropyl)methoxy)-6,8-difluoro-4-((1S,3S,4S)-3-vinyl-2,5-diazabicyclo[2.2.2]octan-2-yl)quinazolin-7-yl)naphthalen-2-ol (Ex. 6a, Peak 1). MS (ESI): m/z (M+H)⁺ 572, 1H NMR (600 MHz, DMSO-d₆) δ 10.03 (s, 1H), 7.89-7.83 (m, 1H), 7.84-7.80 (m, 1H), 7.52-7.40 (m, 1H), 7.37-7.32 (m, 1H), 7.32-7.29 (m, 1H), 7.29-7.22 (m, 1H). 7.17-7.13 (m, 11H), 6.30-6,04 (m, 1H), 5.45 (dd, J=17.0, 11.2 Hz, 1H), 5.33 (t, J=11.0 Hz, 1H), 4.95 (d, J=10.1 Hz, 1H), 4.77 (d, J=11.4 Hz, 1H), 4.31-4.12 (m, 2H), 3.47 (t, J=11.6 Hz, 1H), 3.10-3.04 (m, 2H), 2.25 (q, J=12.8, 12,3 Hz, 2H), 2.17 (s, 6H), 2.0-1.90 (m, 3H), 1.82-1.70 (m, 2H), 0.65-0.61 (m, 2H), 0.42-0.38 (m, 2H). Absolute stereochemistry was confirmed by X-ray crystallography.

Compounds in the table below were synthesized via similar routes to Ex. 6a.

| | | | [M + H]⁺ |
|---|---|---|---|
| Ex. | Structure | Compound Name | Found |
| Ex 6b | 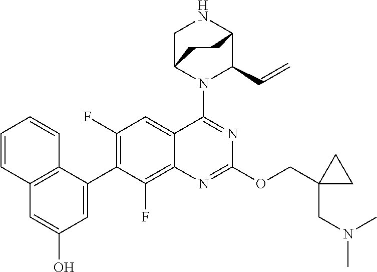 [SMILES: C=C[C@@H]1[C@@H]2CC[C@@H](CN2)N1c1nc(OCC2(CN(C)C)CC2)nc2c(F)c(-c3cc(O)cc4ccccc34)c(F)cc12] | 4-(2-((1- ((dimethylamino)methyl)cyclopropyl)methoxy)-6,8- difluoro-4-((1S,3R,4S)-3-vinyl-2,5- diazabicyclo[2.2.2]octan-2-yl)quinazolin-7- yl)naphthalen-2-ol | 572 |
| | Second eluting peak on reverse phase HPLC | | |
| Ex 7 | 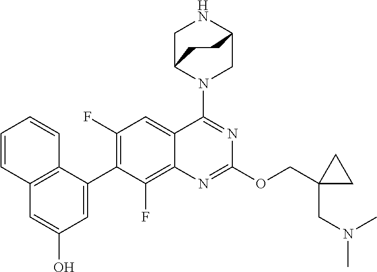 [SMILES: CN(C)CC1(COc2nc(N3C[C@@H]4CC[C@H]3CN4)c3cc(F)c(-c4cc(O)cc5ccccc45)c(F)c3n2)CC1] | 4-(4-((1S,4S)-2,5-diazabicyclo[2.2.2]octan-2-yl)-2-((1- ((dimethylamino)methyl)cyclopropyl)methoxy)-6,8- difluoroquinazolin-7-yl)naphthalen-2-ol | 546 |
| Ex 8a | 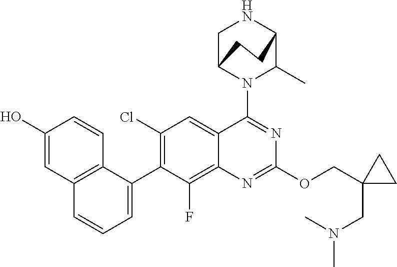 [SMILES: CC1[C@@H]2CC[C@@H](CN2)N1c1nc(OCC2(CN(C)C)CC2)nc2c(F)c(-c3cccc4cc(O)ccc34)c(Cl)cc12] | 4-(6-chloro-2-((1- ((dimethylamino)methyl)cyclopropyl)methoxy)-8- fluoro-4-((1S,4S)-3-methyl-2,5- diazabicyclo[2.2.2]octan-2-yl)quinazolin-7- yl)naphthalen-2-ol | 576 |
| | First eluting peak on reverse phase HPLC | | |
| Ex 8b | 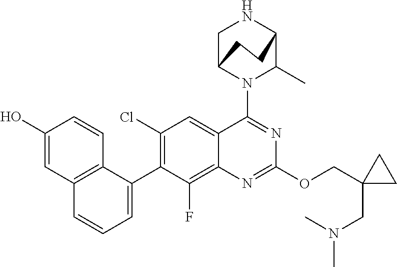 [SMILES: CC1[C@@H]2CC[C@@H](CN2)N1c1nc(OCC2(CN(C)C)CC2)nc2c(F)c(-c3cccc4cc(O)ccc34)c(Cl)cc12] | 4-(6-chloro-2-((1- ((dimethylamino)methyl)cyclopropyl)methoxy)-8- fluoro-4-((1S,4S)-3-methyl-2,5- diazabicyclo[2.2.2]octan-2-yl)quinazolin-7- yl)naphthalen-2-ol | 576 |
| | Second eluting peak on reverse phase HPLC | | |

### Example 9: 4-(2-((1-((dimethylamino)methyl)cyclooropyl)methoxy)-4-((1S,3S,4S)-3-ethyl-2,5-diazabicyclo[2.2.2]octan-2-yl)-6,8-difluoroquinazolin-7-yl)naphthalen-2-ol

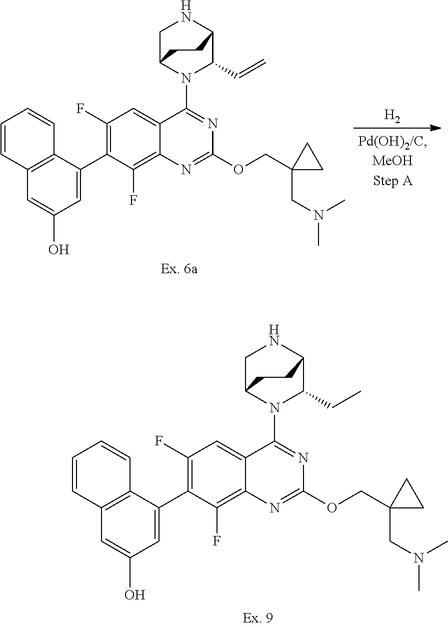 [SMILES: C=C[C@H]1[C@@H]2CC[C@@H](CN2)N1c1nc(OCC2(CN(C)C)CC2)nc2c(F)c(-c3cc(O)cc4ccccc34)c(F)cc12 ; "H2" ; CC[C@H]1[C@@H]2CC[C@@H](CN2)N1c1nc(OCC2(CN(C)C)CC2)nc2c(F)c(-c3cc(O)cc4ccccc34)c(F)cc12]

Palladium hydroxide on carbon (20% loading, 9.7 mg, 0.014 mmol) was added to a mixture of 4-(2-((1-((dimethylamino)methyl)cyclopropyl)methoxy)-6,8-difluoro-4-((1S,3S,4S)-3-vinyl-2,5-diazabicyclo[2.2.2]octan-2-yl)quinazolin-7-yl)naphthalen-2-ol (Ex. 6a) (7.9 mg, 0.014 mmol) in MeOH (690 PL). The reaction was stirred vigorously at room temperature under an atmosphere of Hz for 2 h. The reaction was then filtered through CELITE, washed with MeOH and the filtrate was concentrated under reduced pressure. The crude product was purified by mass triggered reverse phase HPLC (MeCN/water with 0.1% ammonium hydroxide modifier)) to afford 4-(2-((1-((dimethylamino)methyl)cyclopropyl)methoxy)-4-((1S,3S,4S)-3-ethyl-2,5-diazabicyclo[2.2.2]octan-2-yl)-6,8-difluoroquinazolin-7-yl)naphthalen-2-ol (Ex. 9). MS (ESI): m/z (M+H)⁺ 574. ¹H NMR (600 MHz, DMSO-d) δ 10.10 (bs,1H), 7.82 (d, J=8.3 Hz, 1H), 7.58 (dd, J=18,3, 10.5 Hz, 1H), 7.49-7.37 (m, 2H), 7.34-7.22 (m, 2H), 7.16 (dd, J=13.1, 2.2 Hz, 1H), 6.79 (bs, 1H), 4.62-4.58 (m, 1H), 4.31-4.16 (m, 2H), 3.59-3.42 (m, 2H), 3.09 (d, J=10.8 Hz, 1H), 3.03-2.99 (m, 1H), 2.30-2.19 (m, 2H), 2.16 (s, 6H), 1.99-1.95 (m, 1H), 1.85-1.82 (m, 1H), 1.82-1.69 (m, 3H), 1.27-1.21 (m, 1H), 1.01-0.88 (m, 3H), 0.65-0.61 (m, 2H), 0.42-0.38 (s, 2H).

### Example 10: 4-(2-((1-((dimethylamino)methy)cyclopropyl)methoxy)-4-((1S,3R,4S)-3-ethyl-2,5-diazabicyclo[2.2.2]octan-2-yl)-6,8-difluoroquinazolin-7-yl)naphthalen-2-ol

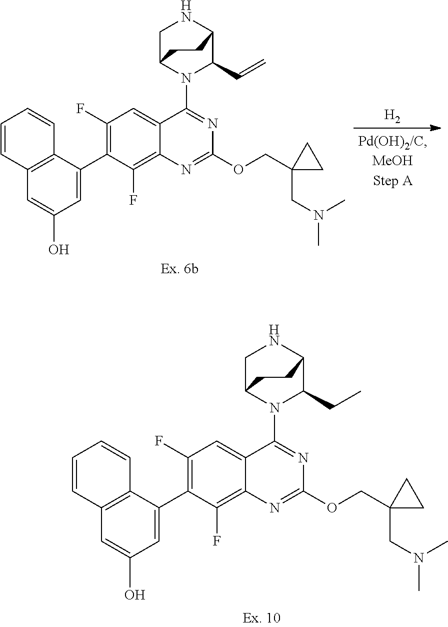 [SMILES: C=C[C@@H]1[C@@H]2CC[C@@H](CN2)N1c1nc(OCC2(CN(C)C)CC2)nc2c(F)c(-c3cc(O)cc4ccccc34)c(F)cc12 ; "H2" ; CC[C@@H]1[C@@H]2CC[C@@H](CN2)N1c1nc(OCC2(CN(C)C)CC2)nc2c(F)c(-c3cc(O)cc4ccccc34)c(F)cc12]

The synthesis of 4-(2-((1-((dimethylamino)methyl)cyclopropyl)methoxy)-4-((1S,3R,4S)-3-ethyl-2,5-diazabicyclo[2,2,2]octan-2-yl)-6,8-difluoroquinazolin-7-yl)naphthalen-2-ol (Ex. 10) followed the same procedure as above for 4-(2-((1-((dimethylamino)methyl)cyclopropyl)methoxy)-4-((1S,3S,4S)-3-ethyl-2,5-diazabicyclo[2.2.2]octan-2-yl)-6,8-difluoroquinazolin-7-yl)naphthalen-2-ol (Ex. 9), using 4-(2-((1-((dimethylamino)methyl)cyclopropyl)methoxy)-6,8-difluoro-4-((1S,3R,4S)-3-vinyl-2,5-diazabicyclo[2.2.2]octan-2-yl)quinazolin-7-yl)naphthalen-2-ol (Ex. 6b) as starting material. MS (ESI): m/z (M+H)⁺ 574. ¹H NMR (600 MHz, DMSO-d₆) δ 7.84-7.80 (m, 1H), 7.52-7.39 (m, 2H), 7.32-7.30 (m, 1H), 7.29-7.22 (m, 2H), 7.17-7.14 (m, 1H), 4.52-4.35 (m, 2H), 4.34-4.18 (m, 2H), 3.03-3.01 (m, 1H), 2.95-2.90 (m, 2H), 2.39-2.29 (m, 2H), 2.28-2.21 (m, 2H), 2.17 (s, 6H), 2.08-1.98 (m, 1H), 1.96-1.75 (m, 4H), 1.46-1.403 (m, 1H), 0.97-0.91 (m, 3H), 0.66-0.62 (m, 2H). 0.43-0.39 (m, 2H).

### Example 11: 4-(4-(3.8-diazabicyclo[3.2.1]octan-3-yl)-6-chloro-2-(((S)-1-((dimethylamino)methyl)-2,2-difluorocyclopropyl)methoxy)-8-fluoroquinazolin-

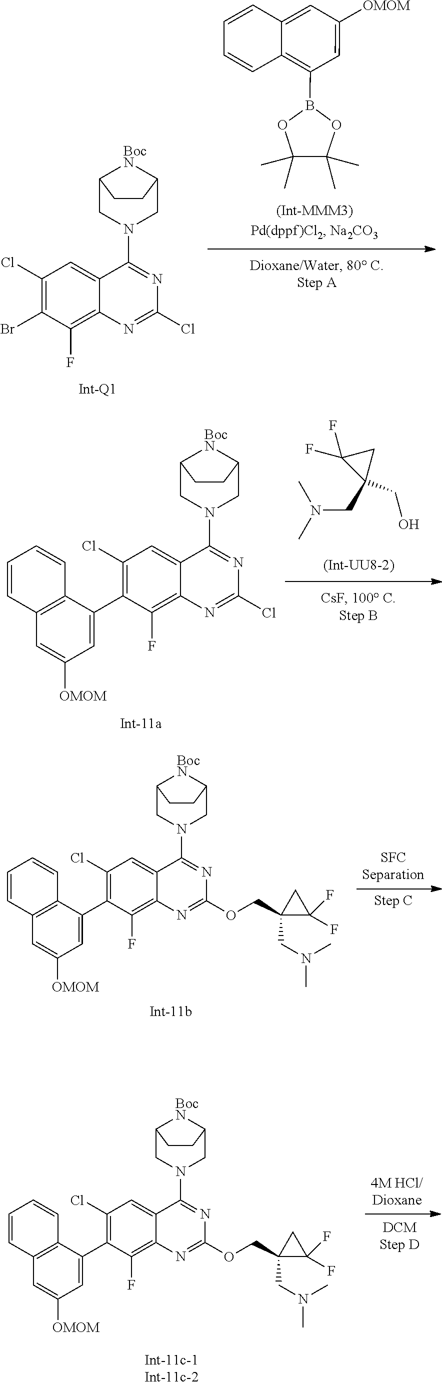

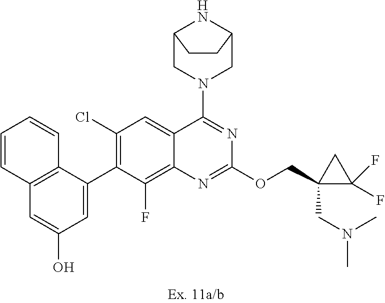 [SMILES: CN(C)C[C@]1(COc2nc(N3CC4CCC(C3)N4)c3cc(Cl)c(-c4cc(O)cc5ccccc45)c(F)c3n2)CC1(F)F]

### Step A: tert-but 326-dichloro-8-fluoro-7-(3-(methoxymethoxy)nahthalen-1-yl)quinazolin-4-yl)-38-diazabicyclo[3.2.1]octane-8-carboxlate (Int-11a)

To a mixture of tert-butyl 3-(7-bromo-2,6-dichloro-8-fluoroquinazolin-4-yl)-3,B-diazabicyclo[3.2.1]octane-8-carboxylate (Int-O1) (0.80 g, 1.3 mmol), 2-(3-(methoxymethoxy)naphthalen-1-1)4,4,5,5-tetrametyl-13,2-dioxaborolane (Int-MMM3) (0.44 g, 1.4 mmol) sodium carbonate (0.34 g, 3.2 mmol)H and [1.1′-bis(diphenylphosphino)ferrocene] dichloropalladium(II) (0.19 g, 0.25 mmol) were added degassed dioxane (8.0 mL) and water (2.0 mL). The mixture was heated to 80° C. for 30 minutes. After 30 minutes, the reaction was allowed to cool to room temperature. Water (10 mL) was added and the mixture was extracted with EtOAc (2×50 mL). The combined organic layers were dried over Na₂SO₄, filtered and concentrated under reduced pressure. The crude residue was purified by silica gel column chromatography (10-20% EtOAc gradient in hexanes) to afford tert-butyl 3-(2,6-dichloro-8-fluoro-7-(3-(methoxymethoxy)naphthalen-1-yl)quinazolin-4-yl)-3,8-diazabicyclo[3.2.1]octane-8-carboxylate (Int-11a). MS (ESI): m/z (M+H)613.

### Step B: tert-butyl 3-(6-chloro-2-(S)-1-(dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11b)

A vial containing tert-butyl 3-(2,6-dichloro-8-fluoro-7-(3-(methoxymethoxy)naphthalen-1-yl)quinazolin-4-yl)-3,8-diazabicyclo[3.2.1]octane-8-carboxylate (Int-1α) (30 mg, 0.049 mmol), (S)₄₁-((dimethylamino)methyl)-2,2-difluorocyclopropyl)methanol (Int-UU8-2) (16 mg, 0.098 mmol), and cesium fluoride (37 mg, 0.24 mmol) in DMSO (0.35 mL) was heated to 100° C. for 18 h. After 18 h, the mixture was allowed to cool to room temperature and then diluted with ethanol (2.0 mL) and water (0.25 mL). The resulting mixture was filtered and the filtrate was purified by preparative reverse phase HPLC (MeCN/water with 0.1% TFA modifier) to afford tert-butyl 3-(6-chloro-2-(((S)-1-((dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11 b). MS (ESI): m/z (M+H)⁺ 742.

### Step C: tert-butyl 3-(6-chloro-2-(((S)-1-((dimethylamino)methyl)-2,2-difluorocyclopropyl)methoxy)-8-fluoro-7-(34methoxymethoxy)naphthalen-1-yl)quinazolin-4-yl)-3,8-diazabicyclo[3.2.1]octane-8-carboxylate (Int-11c-1 and Int11c-2)

An atropisomeric mixture of tert-butyl 3-(6-chloro-2-(((S)-1-((dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11b) was resolved using chiral SFC resolution (Column E; Conditions: 35% MeOH w/0.1% NH₄OH) to afford tert-butyl 3-(6-chloro-2-(((S)-1-((dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11c-1, Peak 1), MS (ESI): m/z (M+H)₄ 742, and tert-butyl 3-(6-chloro-2-(((S)-1-((dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11c-2, Peak 2). MS (ESI): m/z (M+H)⁺ 742.

### Step D: 4-(4-(3.8-diazabicyclo[3.2.1]octan-3-yl)-6-chloro-2-(((S)-1-((dimethylamino)methyl)-2,2-difluorocyclopropyl)methoxy)-8-fluoroquinazolin-7-yl)naphthalen-2-ol (Ex. 11a)

To a mixture of tert-butyl 3-(6-chloro-2-(((S)-1-((dimethylamino)methyl)-2,2-difluorocyclopropyl)methoxy)-8-fluoro-7-(3-(methoxymethoxy)naphthalen-1-yl)quinazolin-4-yl)-3,8-diazabicyclo[3.2.1]octane-8-carboxylate (Int-11c-1) (11 mg, 0.015 mmol) in 2-propanol (1.0 mL) was added HCl (4.0 M in dioxane, 0.50 mL, 2.0 mmol) at room temperature. The mixture was stirred at rt for 24 h and then concentrated under reduced pressure. The resulting residue was diluted in ethanol (2.0 mL) and filtered. The filtrate was purified by preparative reverse phase HPLC (MeCN/water with 0.1% TFA modifier) to afford 4-(4-(3.8-diazabicyclo[3.2.1]octan-3-yl)-6-chloro-2-(((S)-1-((dimethylamino)methyl)-2,2-difluorocyclopropyl)methoxy)-8-fluoroquinazolin-7-yl)naphthalen-2-ol (Ex. 11a). MS (ESI): m/z (M+H)⁺ 598. ¹H NMR (500 MHz. Methanol-d₆) δ 8.06 (s, 1H), 7.79 (d, J=8.4 Hz, 1H), 7.53-7.36 (m, 1H), 7.30 (d, J=2.2 Hz, 1H), 7.28-7.15 (m, 2H), 7.06 (d, J=2.4 Hz, 1H), 4.79 (d, J=12.9 Hz, 3H), 4.68 (d, 1=12.8 Hz, 1H), 4.27 (s, 2H), 3.93 (d, J=14.1 Hz, 2H), 3.76 (d, J=14.1 Hz, 1H), 3.48 (d, J=14.1 Hz, 1H), 3.02 (s, 6H), 2.17 (s, 4H), 2.08-1.97 (m, 1H), 1.90-1.80 (m, 1H).

4-(4-(3.8-diazabicyclo[3.2.1]octan-3-yl)-6-chloro-2-(((S)-1-((dimethylamino)methyl)-2,2-difluorocyclopropyl)methoxy)-8-fluoroquinazolin-7-yl)naphthalen-2-ol (Ex. 11b) was synthesized using the same procedure as Ex. 11a, using Int-11c-2. MS (ESI): m/z (M+H)⁺ 598. ¹H NMR (500 MHz, Methanol-d₆) δ 8.06 (s, 1H), 7.79 (d, J=8.3 Hz, 1H), 7.51-7.36 (m, 1H), 7.30 (d, J=2.2 Hz, 1H), 7.22 (q, J=9.0, 8.4 Hz, 2H), 7.06 (d, J=2.4 Hz, 1H), 4.78 (t, J=11.4 Hz, 3H), 4.67 (d, 1=12.8 Hz, 1H), 4.27 (s, 2H), 3.93 (d,1=14.3 Hz, 2H), 3.75 (d, J=13.9 Hz, 1H), 3.49 (d, J=14.1 Hz, 1H), 3.02 (s, 6H), 2.17 (s, 4H), 2.10-1.95 (m, 1H), 1.86 (t. J=10.8 Hz, 1H).

The compounds in the table below were prepared using the Int-11b described in the procedure above using similar conditions.

| | | | [M + H]⁺ | Purification |
|---|---|---|---|---|
| Ex. | Structure | Compound Name | Found | conditions |
| Ex.108 | 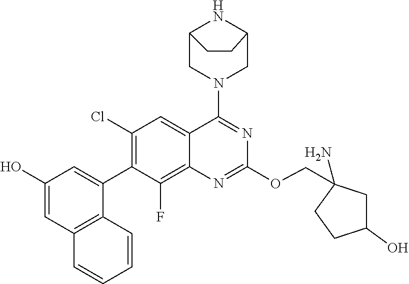 [SMILES: NC1(COc2nc(N3CC4CCC(C3)N4)c3cc(Cl)c(-c4cc(O)cc5ccccc45)c(F)c3n2)CCC(O)C1] | 4-(2-((1-amino-3- hydroxycyclopentyl) methoxy)-4-(3,8- diazabicyclo[3.2.1] octan-3-yl)-6-chloro-8- fluoroquinazolin-7- yl)naphthalen-2-ol, TFA | 564 | -NA- |
| | Mixture | | | |
| Ex.109 | 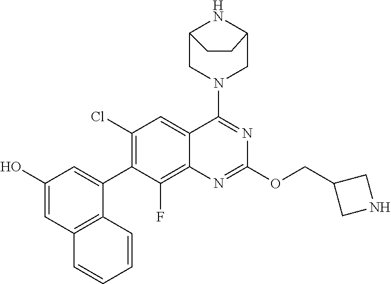 [SMILES: Oc1cc(-c2c(Cl)cc3c(N4CC5CCC(C4)N5)nc(OCC4CNC4)nc3c2F)c2ccccc2c1] | 4-(2-(azetidin-3- ylmethoxy)-4-(3,8- diazabicyclo[3.2.1] octan-3-yl)-6-chloro-8- fluoroquinazolin-7- yl)naphthalen-2-ol, TFA | 520 | -NA- |
| | mixture | | | |
| Ex.110 | 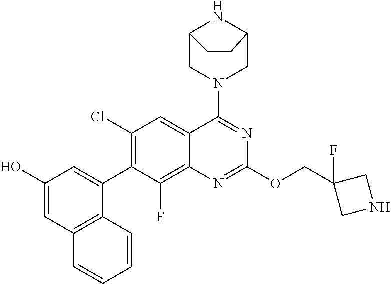 [SMILES: Oc1cc(-c2c(Cl)cc3c(N4CC5CCC(C4)N5)nc(OCC4(F)CNC4)nc3c2F)c2ccccc2c1] | 4-(4-(3,8-diazabicyclo [3.2.1]octan-3-yl)- 6-chloro-8-fluoro-2- ((3-fluoroazetidin-3- yl)methoxy)quinazolin- 7-yl)naphthalen-2- ol, TFA | 538 | -NA- |
| | mixture | | | |
| Ex.111 | 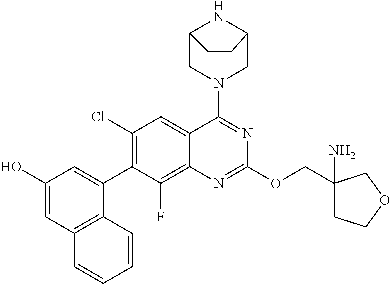 [SMILES: NC1(COc2nc(N3CC4CCC(C3)N4)c3cc(Cl)c(-c4cc(O)cc5ccccc45)c(F)c3n2)CCOC1] | 4-(2-((3-amino- tetrahydrofuran-3- yl)methoxy)-4- (3,8-diazabicyclo[3.2.1] octan-3-yl)-6-chloro-8- fluoroquinazolin-7-yl) naphthalen-2-ol, TFA | 550 | -NA- |
| | mixture | | | |
| Ex.112 | 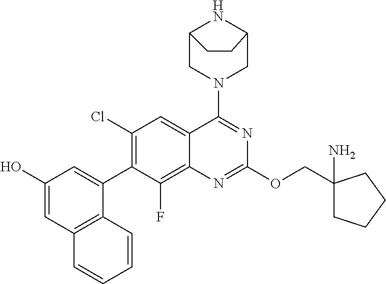 [SMILES: NC1(COc2nc(N3CC4CCC(C3)N4)c3cc(Cl)c(-c4cc(O)cc5ccccc45)c(F)c3n2)CCCC1] | 4-(2-((1-aminocyclo- pentyl)methoxy)-4- (3,8-diazabicyclo[3.2.1] octan-3-yl)-6-chloro-8- fluoroquinazolin-7- yl)naphthalen-2-ol, TFA | 548 | -NA- |
| | mixture | | | |
| Ex.113 | 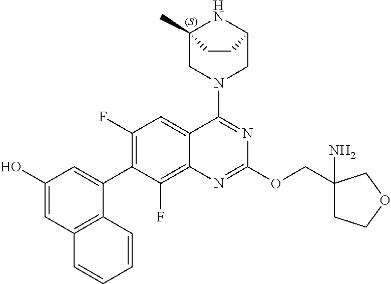 | 4-(2-((3-amino- tetrahydrofuran-3- yl)methoxy)-6,8- difluoro-4-((1S,5R)-1- methyl-3,8-diazabicyclo [3.2.1]octan-3-yl)quinazolin- 7-yl)naphthalen-2-ol, TFA | 548 | -NA- |
| | Mixture-(3-furan) | | | |
| Ex.114 | 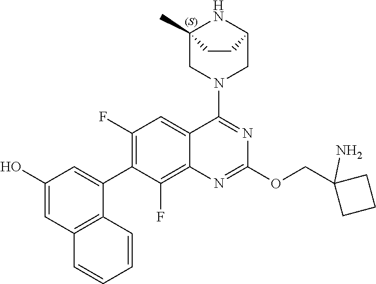 | 4-(2-((1-aminocyclobutyl) methoxy)-6,8-difluoro- 4-((1S,5R)-1-methyl-3,8- diazabicyclo[3.2.1] octan-3-yl)quinazolin- 7-yl)naphthalen-2-ol, TFA | 532 | -NA- |
| | mixture | | | |

### Example 12: 4-(24((1S,5S)-2-(dimethylamino)bicyclo[3.1.0]hexan-1-yl)methoxy)-6,8-difluoro-44(1S,5R)-1-methyl-3,8-diazabicyclo[3.2.1]octan-3-yl)quinazolin-7-yl)naphthalen-2-ol (Ex. 12a/b)

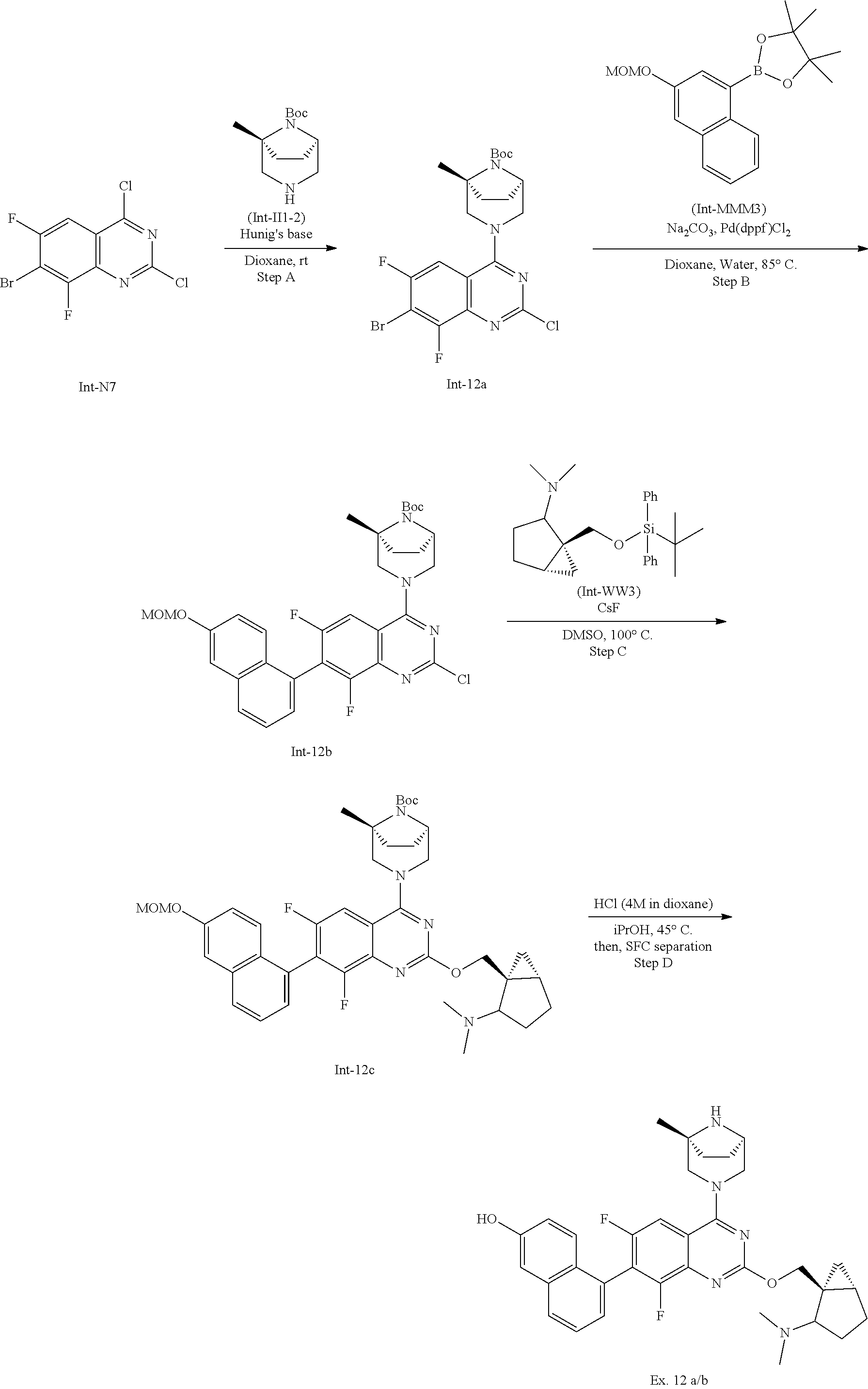

### Step A; tert-butyl (1S,5R)-3-(7-bromo-2-chloro-6,8-difluoroquinazolin-4-yl)-1-methyl-3,8-diazabicyclo[3.2.1]octane-8-carboxylate (Int-12a)

To a mixture of 7-bromo-2,4-dichloro-6,8-difluoroquinazoline (Int-N7) (0.30 g, 0.96 mmol) and tert-butyl (1S,5R)-1-methyl-3,8-diazabicyclo[3.2.1]octane-8-carboxylate (Int-III-2) (0.26 g, 1.1 mmol) in dioxane (7.0 mL) was added N-ethyl-N-isopropylpropan-2-amine (0.50 mL, 2.9 mmol) and the mixture was stirred at room temperature for 2.5 hours. The reaction was then diluted with cold water and sonicated for 30 minutes. The mixture was filtered and the solids were collected via filtration and then dried under vacuum to afford tert-butyl (1S,5R)-3-(7-bromo-2-chloro-6,8-difluoroquinazolin-4-yl)-1-methyl-3,8-diazabicyclo[3.2.]octane-8-carboxylate (Int-12a) that was used in subsequent transformations without further purification. MS (ESI)- m/z (M+H)⁺ 503, 505.

### Step B: tert-butyl (1S,5R)-3-(2-chloro-6,8-difluoro-7-(3-(methoxymethoxy)naphthalen-1-yl)quinazolin-4-yl)-1-methyl-3,8-diazabicyclo[3.2.1]octane-8-carboxylate (Int-12b)

To a mixture of tert-butyl (1S,R)-3-(7-bromo-2-chloro-6,8-difluoroquinazolin-4-yl)-1-methyl-3,8-diazabicyclo[3.2.]octane-8-carboxylate (Int-12a) (0.30 g, 0.60 mmol), 2-(3-(methoxymethoxy)naphthalen-1-yl)-4,4,⁵,5-tetramethyl-1,3,2-dioxaborolane (Int-MMM3) (0.19 g, 0.60 mmol) and PdCl₂(dppf)-DCM adduct (49 mg, 0.060 mmol) in dioxane (6.0 mL) was added sodium carbonate (1.0 M in water, 1.5 mL, 1.5 mmol). The mixture was heated to 65° C. for 2.5 hours. After 2 hours, the mixture was allowed to cool to room temperature. Water was added and the mixture was extracted with ethyl acetate (2×). The organic layers were combined, washed with brine, dried over MgSO₄, filtered and then concentrated under reduced pressure. The resulting crude residue was purified by silica gel column chromatography (0-75% ethyl acetate gradient in hexanes) to afford tert-butyl (1S,5R)-3-(2-chloro-6,8-difluoro-7-(3-(methoxymethoxy)naphthalen-1-yl)quinazolin-4-yl)-1-methyl-3,8-diazabicyclo[3.2.1]octane-8-carboxylate (Int-12b). MS (ESI): m/z (M+H)⁺ 611.

### Step C: tert-butyl (1S,5R)-3-(2-((1S,5S)-2-(dimethylamino)bicyclo[3.1.0]hexan-1-yl)methoxy)-6,8-difluoro-7-(3-(methoxymethoxy)naphthalen-1-yl)quinazolin-4-yl-1-methyl-3,8-diazabicyclo[3.2.1]octane-8-carboxylate (Int-12c)

To a vial containing tert-butyl (1S,5R)-3-(2-chloro-6,8-difluoro-7-(3-(methoxymethoxy)naphthalen-1-yl)quinazolin-4-yl)-1-methyl-3,8-diazabicyclo[3.2.1]octane-8-carboxylate (Int-12b) (32 mg, 0.052 mmol) and DMSO (1.0 mL) were added (1S,5S)-1-(((tert-butyldiphenylsilyl)oxy)methyl)-N,N-dimethylbicyclo[3.1.0]hexan-2-amine (Int-WW3) (21 mg, 0.052 mmol) and cesium fluoride (32 mg, 0.21 mmol). The mixture was heated to 100° C. for 18 h. After 18 h, the mixture was allowed to cool to room temperature, diluted with water, and extracted with ethyl acetate (3×). The combined organic layers were washed with brine, dried over MgSO₄, filtered and then concentrated under reduced pressure to afford tert-butyl (1S5R-3-2-(((1S,5S-2-(dimethylamino)bicyclo[3.1.0]hexan-1-yl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (Int-12c) which was used in subsequent transformations without further purification.

### Step D: 4-(2-(((1S,5S)-2-(dimethylamino)bicyclo[3.1.0]hexan-1-yl)methoxy)-6,8-difluoro-4-((1S,5R)-1-methyl-3,8-diazabicyclo[3.2.1]octan-3-yl)quinazolin-7-yl)naphthalen-2-ol (Ex. 12a/b)

To a vial containing tert-butyl (1S,5R)-3-(2-(((1S,5S)-2-(dimethylamino)bicyclo[3.1.0]hexan-1-yl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (Int-12c) (38 mg, 0.052 mmol) were added isopropanol (1.0 mL) and then HCl (4.0 M in dioxane, 0.45 mL, 1.8 mmol). The mixture was heated to 45° C. for 3 hours. After 3 hours, the mixture was allowed to cool to room temperature and then concentrated under reduced pressure. The crude mixture was purified by reverse phase prep-HPLC (MeCN/water with 0.1% TFA modifier) to afford 4-(2-(((1S,5S)-2-(dimethylamino)bicyclo[3.1.0]hexan-1-yl)methoxy)-6,8-difluoro-4-((1S,5R)-1-methyl-3,8-diazabicyclo[3.2.1]octan-3-yl)quinazolin-7-yl)naphthalen-2-ol (Ex. 12a, Peak 1) MS (ESI): m/z (M+H)⁺ 586. ¹H NMR (500 MHz, DMSO-de) δ 9.66 (s, 1H), 7.86-7.82 (m, 2H), 7.50-7.45 (m, 1H), 7.34 (d, J=2.1 Hz, 1H), 7.32 (s, 1H), 7.28 (q, J=8.1, 6.9 Hz, 2H), 7.15 (d, J=2,3 Hz, 1H), 4.72-4.67 (m, 1H), 4.55-4.50 (m, 1H), 4.40-4.35 (m, 1H), 4.34-4.30 (m,I 1H), 4.26-4.21 (m, 1H), 4.14-4.07 (m, 1H), 3.69-3.62 (m, 2H), 2.91-2.86 (m, 3H), 2.85-2.80 (m, 3H), 2.14-2.07 (m, 2H), 2.05-1.98 (m, 2H), 1.91-1.81 (m, 2H), 1.78-1.71 (m, 2H), 1.60-1.55 (m, 1H), 1.51 (s, 3H), 1.18-1.13 (m, 1H), 0.99-0.94 (m, 1H). And 4-(2-(((1S,5S)-2-(dimethylamino)bicyclo[3.1.0]hexan-1-yl)methoxy)-6,8-difluoro-4-((1S,5R)-1-methyl-3,8-diazabicyclo[3.2.1]octan-3-yl)quinazolin-7-yl)naphthalen-2-ol (Ex. 12b, Peak 2) MS (ESI): m/z (M+H)⁺ 586.

¹H NMR (500 MHz, DMSO-d₆) δ 9.66 (s, 1H), 7.86-7.82 (m, 2H), 7.50-7.46 (m, 1H), 7.33 (d, J=2.0 Hz, 1H), 7.30-7.25 (m, 2H), 7.15 (d, J=2.2 Hz, 1H), 4.72-4.67 (m, 1H), 4.55-4.50 (m, 1H), 4.40-4.35 (m, 1H), 4.34-4.30 (m, 1H) 4.26-4.21 (m, 1HI), 4.14-4.07 (m, 1H) 3.70-3.62 (m, 2H), 2.91-2.87 (m, 3H), 2.85-2.80 (m, 3H), 2.14-2.07 (m, 2H), 2.04-1.97 (m, 2H), 1.90-1.81 (m, 2H), 1.78-1.71 (m, 2H), 1.59-1.55 (m, 1H), 1.51 (s, 3H), 1.18-1.13 (m, 1H), 0.99-0.93 (m, 1H).

Examples in the table below were synthesized in a similar manner as Ex. 12.

| | | | | | |
|---|---|---|---|---|---|
| Ex.115 | | 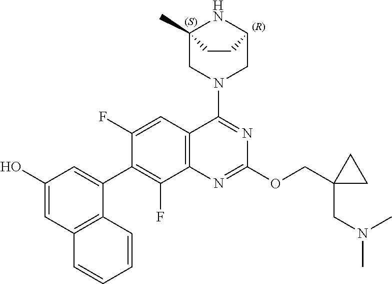 | 4-(2-((1- ((dimethylamino)methyl) cyclopropyl)methoxy)- 6,8-difluoro-4-((1S,5R)- 1-methyl-3,8- diazabicyclo [3.2.1]octan-3-yl) quinazolin-7-yl) naphthalen-2-ol | 560.3 | NA |
| Ex.116a | SFC after Negishi- Peak 2 Column D; Condition 0.1% NH3H2O ETOH | 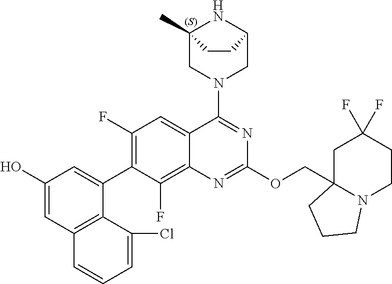 | 5-chloro-4-(2-((7,7- difluorohexahydro- indolizin-8a(1H)- yl)methoxy)-6,8- difluoro-4-((1S,5R)-1- methyl-3,8- diazabicyclo[3.2.1]octan- 3-yl)quinazolin-7- yl)naphthalen-2-ol | 656 | Column K; Condition: 0.1% NH3H2O IPA |
| | | Peak 1, SFC after | | | |
| | | final deprotection | | | |
| Ex.116b | SFC after Negishi- Peak 2 Column D; Condition 0.1% NH3H2O ETOH | 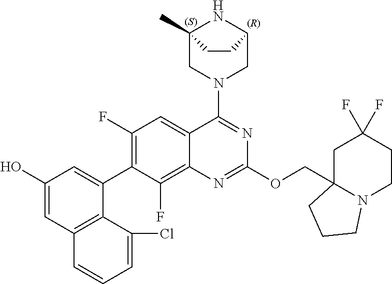 | 5-chloro-4-(2-(((S)-7,7- difluorohexahydro- indolizin-8a(1H)- yl)methoxy)-6,8- difluoro-4-((1S,5R)-1- methyl-3,8-diazabicyclo [3.2.1]octan-3- yl)quinazolin-7- yl)naphthalen-2-ol | 656 | Column K; Condition: 0.1% NH3H2O IPA |
| | | Peak 2, SFC after final | | | |
| | | deprotection | | | |

### Example 13: 2-(((4-(3.8-diazabicyclo[3.2.1]octan-3-yl)-6,8-difluoro-7-(3-hydroxynaphthalen-1-yl)quinazolin-2-yl)ox)methyl)cyclopropane-1-carboxylc acid (Ex. 13)

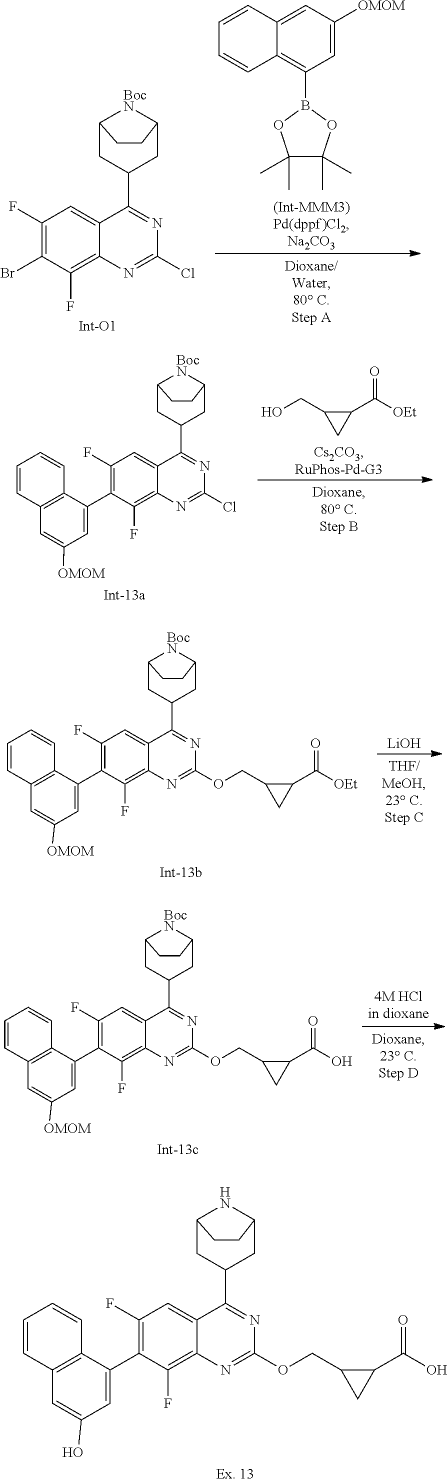

### Step A: tert-butyl 3-(2-chloro-6,8-difluoro-7-(3-(methoxymethoxy)naphthalen-1-yl)quinazolin-4-yl)-3,8-diazabicyclo[3.2.1]octane-8-carboxylate (Int-13a)

To a mixture of tert-butyl 3-(7-bromo-2-chloro-6,8-difluoroquinazolin-4-yl)-3,8-diazabicyclo[3.2.1]octane-8-carboxylate (0.36 g, 0.74 mmol) (Int-O1) in 1,4-dioxane (12 mL) were added 2-(3-(methoxymethoxy)naphthalen-1-yl)-4,4,5,5-tetramethyl-1,3,2-dioxaborolane (Int-MMM3) (0.19 g, 0.59 mmol), sodium carbonate (0.20 g, 1.8 mmol), water (2.4 mL) and [1.1′-bis(diphenylphosphino)ferrocene]dichloropalladium(II) (54 mg, 0.074 mmol). The reaction was heated to 80° C. for 1.5 h. After 1.5 h, the mixture was allowed to cool to room temperature. Water (20 mL) was added and the mixture was extracted with EtOAc (20 mL×3). The combined organic layers were dried over Na₂SO₄, filtered, and concentrated under reduced pressure. The crude material was purified by flash silica gel chromatography (0-20% ethyl acetate gradient in Pet, ether) to afford tert-butyl 3-(2-chloro-6,8-difluoro-7-(3-(methoxymethoxy)naphthalen-1-yl)quinazolin-4-yl)-3,8-diazabicyclo[3.2.1]octane-8-carboxylate (Int- 13a). MS (ESI): m/z (M+H)Y 597.

### Step B: tert-butyl 3-(2-((2-(ethoxycarbonyl)cyclopropyl)methoxy)-6,8-difluoro-7-(3-(methoxymethoxy)naphthalen-1-yl)quinazolin-4-yl)-3,8-diazabicyclo[3.2.1]octane-8-carboxylate (Int-13b)

To a vial containing tert-butyl 3-(2-chloro-6,8-difluoro-7-(3-(methoxymethoxy)naphthalen-1-yl)quinazolin-4-yl)-3,8-diazabicyclo[3.2.1]octane-8-carboxylate (Int-13a) (50 mg, 0.084 mmol) were added ethyl 2-(hydroxymethyl)cyclopropane-1-carboxylate (24 mg, 0.17 mmol), (2-dicyclohexylphosphino-2′,6′-diisopropoxy-1,1′-biphenyl)[2-(2′-amino-1,1′-biphenyl)]palladium(II) methanesulfonate (RuPhos-Pd-G3) (7.0 mg, 8.4 μmol) and then 1.4-dioxane (0.60 mL). Cesium carbonate (82 mg. 0.25 mmol) was added and the mixture was sparged for two minutes with nitrogen. The mixture was heated to 80° C. for 6 h, cooled to room temperature and was then filtered and purified via reverse phase column chromatography (20-100% MeCN gradient in water with a 0.05% TFA modifier to afford tert-butyl 3-(2-((2-(ethoxycarbonyl)cyclopropyl)methoxy)-6,8-difluoro-7-(3-(methoxymethoxy)naphthalen-1-yl)quinazolin-4-yl)-3,8-diazabicyclo[3.2.1]octane-8-carboxylate (Int-13b). MS (ESI): m/z (M+H)⁺ 705.

### Step C: 2-(((4-(8-(tert-butoxycarbonyl)-3,8-diazabicyclo[3.2.1]octan-3-yl)-6,8-difluoro-7-(3-(methoxymethoxylnaphthalen-1-yl)quinazolin-2-yl)oxy)methyl)cyclopropane-1-carboxylc acid (Int. 13c)

To a mixture of tert-butyl 3-(2-((2-(ethoxycarbonyl)cyclopropyl)methoxy)-6,8-difluoro-7-(3-(methoxymethoxy)naphthalen-1-yl)quinazolin-4-yl)-3,8-diazabicyclo[3.2.1]octane-8-carboxylate (Int-13b), tetrahydrofuran (0.20 mL) and MeOH (0.13 mL) at room temperature was added lithium hydroxide (2.0 M in water, 85 μL, 0.17 mmol). The mixture was heated at 50° C. for 2 hours. After 2 hours, the mixture was allowed to cool to room temperature and then aqueous HCl (1 N) was added until the pH reached 6-7. The mixture was then extracted with EtOAc (2×10 mL). The combined organic layers were dried over Na₂SO₄, filtered and concentrated under reduced pressure to afford 2-(((4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)oxy)methyl)cyclopropane-1-carboxylc acid (Int-13c) that was used in subsequent transformations without further purification. MS (ESI): m/z (M+H)⁺ 677.

### Step D: 2-(((4-(3.8-diazabicyclo[3.2.1]octan-3-yl)-6,8-difluoro-7-(3-hydroxynaphthalen-1-yl)quinazolin-2-yl)oxy)methyl)cyclopropane-1-carboxylc acid (Ex. 13)

To a mixture of 2-(((4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)oxy)methyl)cyclopropane-1-carboxylc acid (Int-13c) (38 mg, 0.051 mmol) and 1.4-dioxane (0.10 mL) was added HCl (4.0 M in dioxane, 0.13 mL, 0.52 mmol) and the mixture was stirred at room temperature for 18 hours. After 18 hours, the solid was filtered and washed with ether (2×10 mL) to afford 2-(((4-(3.8-diazabicyclo[3.2.1]octan-3-yl)-6,8-difluoro-7-(3-hydroxynaphthalen-1-yl)quinazolin-2-yl)oxy)methyl)cyclopropane-1-carboxylc acid (Ex. 13). MS (EST): m/z (M+H)⁺ 533. ¹H NMR (500 MHz, methanol-d₆) δ 7.97 (d, J=9.5 Hz, 1H), 7.80 (d, J=8.1 Hz, 1H), 7.52-7.44 (m, 1H), 7.41 (d, J=7.9 Hz, 1H), 7.34 (d, J=2,3 Hz, 1H), 7.28 (t, J=7.4 Hz, 1H), 7.18 (d, J=2,3 Hz, 1H), 5.11-4.95 (m, 2H), 4.84-4.74 (m, 1H), 4.48 (d, J=12.5 Hz, 1H), 4.36 (s, 2H), 4.20 (m, 2H), 2.29-2.08 (m, 4H), 2.01 (s, 1H), 1.84 (dd, J=8.3, 4.1 Hz, 1H), 1.33 (dt, J=9.1, 4.6 Hz. 1H), 1.24-1.13 (m, 1H).

### Example 14: 4-(4-(3.8-diazabicyclo[3.2.1]octan-3-yl)-6,8-difluoro-2-(2,5-diazaspiro[3.4]octan-2-yl)quinazolin-7-yl)naphthalen-2-ol (Ex. 14)

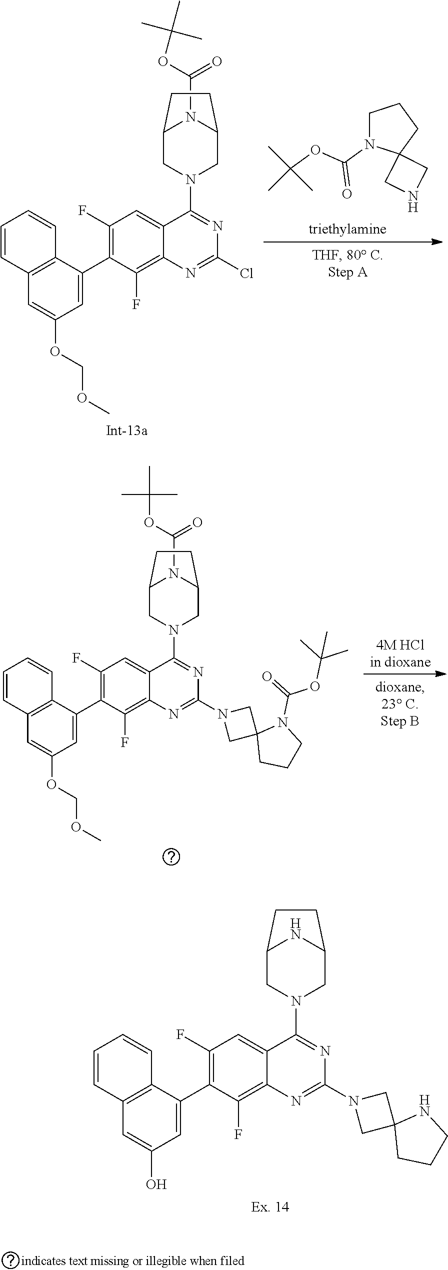 [SMILES: COCOc1cc(-c2c(F)cc3c(N4CC5CCC(C4)N5C(=O)OC(C)(C)C)nc(Cl)nc3c2F)c2ccccc2c1 ; CC(C)(C)OC(=O)N1CCCC12CNC2 ; COCOc1cc(-c2c(F)cc3c(N4CC5CCC(C4)N5C(=O)OC(C)(C)C)nc(N4CC5(CCCN5C(=O)OC(C)(C)C)C4)nc3c2F)c2ccccc2c1 ; Oc1cc(-c2c(F)cc3c(N4CC5CCC(C4)N5)nc(N4CC5(CCCN5)C4)nc3c2F)c2ccccc2c1]

### Step A: tert-butyl 2-(4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)-2,5-diazaspiro[3.4]octane-5-carboxylate (Int-14a)

To a vial containing tert-butyl 3-(2-chloro-6,8-difluoro-7-(3-(methoxymethoxy)naphthalen-1-yl)quinazolin-4-yl)-3,8-diazabicyclo[3.2.1]octane-8-carboxylate (Int-13a) (25 mg, 0.042 mmol) were added THF (1.5 mL), tert-butyl 2,5-diazaspiro[3.4]octane-5-carboxylate (18 mg, 0.084 mmol) and then triethylamine (0.018 mL, 0.13 mmol). The mixture was heated at 80° C. for 18 hours. After 18 hours, the mixture was concentrated under reduced pressure and then purified by reverse phase column chromatography (10-100% MeCN gradient in water with a 0.05% TFA modifier) to afford tert-butyl 2-(4-(8-(tert-butoxycarbonyl)-3,8-diazabicyclo[3.2.]octan-3-yl)-6,8-difluoro-7-(3-(methoxymethoxy)naphthalen-1-yl)quinazolin-2-yl)-2,5-diazaspiro[3.4]octane-5-carboxylate (Int-14a). MS (EST): m/z (M+H)⁺ 773.

### Step B: 4-(4-(3.8-diazabicyclo[3.2.1]octan-3-yl)-6,8-difluoro-2-(2,5-diazaspiro[3.4]octan-2-yl)quinazolin-7-yl)naphthalen-2-ol (Ex. 14)

To a vial containing tert-butyl 2-(4-(8-tert-butoxycarbonyl)-3,8-diazabicyclo[3.2.1]octan-3-yl)-6,8-difluoro-7-(3-(methoxymethoxy)naphthalen-1-yl)quinazolin-2-yl)-2,5-diazaspiro[3.4]octane-5-carboxylate (Int-14a) (25 mg, 0.029 mmol) in dioxane (0.10 mL) was added HCl (4.0 M in dioxane, 73 μL, 0.29 mmol). The mixture was stirred at room for 18 hours. After 18 hours, the solid was collected via filtration and purified via reverse phase column chromatography (10-100% MeCN gradient in water with a 0.05% TFA modifier) to afford 4-(4-(3.8-diazabicyclo[3.2.1]octan-3-yl)-6,8-difluoro-2-(2,5-diazaspiro[3.4]octan-2-yl)quinazolin-7-yl)naphthalen-2-ol (Ex. 14). MS (ESI): m/z (M+H). 529, 1H NMR (500 MHz, Methanol-d₆) δ 7.79 (d, J=8.0 Hz, 1H), 7.65 (d, J=10.0 Hz, 1H), 7.46 (m, 1H), 7.37 (d, J=8.4 Hz, 1H), 7.31 (s, 1H), 7.25 (m, 1H), 7.13 (s, 1H), 4.66 (s, 2H), 4.58 (d, J=10.4 Hz. 2H). 4.43 (d, J=10.1 Hz, 2H), 4.26 (s, 2H), 3.86 (m, 2H), 3.46 (m, 2H), 2.38 (m, 2H), 2.19 (m, 6H).

### Example 15: 3-(((4-(3.8-diazabicyclo[3.2.1]octan-3-yl)-6,8-difluoro-7-(3-hydroxynaphthalen-1-yl)quinazolin-2-yl)oxy)methyl)azetidine-3-carboxamide (Ex. 15)

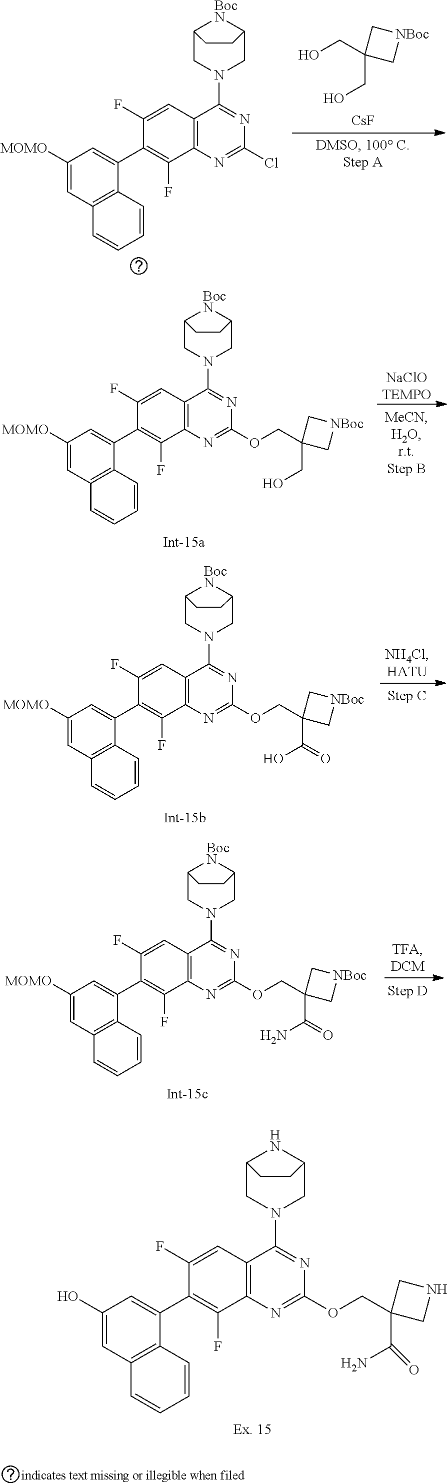

### Step A: tert-butyl 3-(2-(1-(tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-15a)

To a mixture of tert-butyl 3-(2-chloro-6,8-difluoro-7-(3-(methoxymethoxy)naphthalen-1-yl)quinazolin-4-yl)-3,8-diazabicyclo[3.2.1]octane-8-carboxylate (Int-13a) (210 mg, 0.352 mmol) and tert-butyl 3.3-bis(hydroxymethyl)azetidine-1-carboxylate (153 mg, 0.703 mmol) in DMSO (2.1 mL) was added CsF (214 mg, 1.41 mmol) at 20° C. under nitrogen. After the addition was finished, the mixture was heated to 100° C. for 16 h. After heating for 16 h, the mixture was allowed to cool to room temperature and then quenched with water (10 mL), and extracted with EtOAc (30×5). The combined organic layers were washed with brine (5 mL), dried over anhydrous Na₂SO₄, filtered and the filtrate was concentrated under reduced pressure. The crude product was purified by flash silica gel chromatography (0-50% ethyl acetate gradient in petroleum ether) to afford tert-butyl 3-(2-((1-tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-15a). MS (ESI) m/z: (M+H)⁺ 778.

### Step B: 1-(tert-butoxycarbonyl)-3-(((4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)oxy)methyl)azetidine-3-carboxylc acid (Int-15b)

To a mixture of tert-butyl 3-(2-((1-(tert-butoxycarbonyl)-3-(hydroxymethyl)azetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-15a) (125 mg, 0.161 mmol) in water (2.5 mL) and MeCN (2.5 mL) was added (2,2,6,6-tetramethylpiperidin-1-yl)oxyl (TEMPO) (2.5 mg, 0.016 mmol). The mixture was cooled to 10° C. and then sodium hypochlorite (8.0% in water, 0.45 g, 0.50 mL) was added slowly while maintaining the internal temperature between 15° C. to 20° C. After the addition, the resulting mixture was stirred at 20° C. The reaction mixture was then purified by reverse phase prep-HPLC (MeCN/water with 0.2% formic acid modifier)) to afford 1-(tert-butoxycarbonyl)-3-(((4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)oxy)methyl)azetidine-3-carboxylc acid (Int-15b). MS (ESI): m/z (M+H)⁺ 792.

### Step C: tert-butyl 3-(2-((1-(tert-butoxycarbonyl)-3-carbamoylazetidin-3-yl)methoxy)-6,8-difluoro-7-(3-(methoxymethoxy)naphthalen-1-yl)quinazolin-4-yl)-3,8-diazabicyclo[3.2.1]octane-8-carboxylate (Int-15c)

To a mixture of 1-(tert-butoxycarbonyl)-3-(((4-(8-(tert-butoxycarbonyl)-3,8-diazabicyclo[3.2.1]octan-3-yl)-6,8-difluoro-7-(3-(methoxymethoxy)naphthalen-1-yl)quinazolin-2-yl)oxy)methyl)azetidine-3-carboxylc acid (Int-15b) (25 mg, 0.032 mmol) in DMF (2.0 mL) were added 1-[Bis(dimethylamino)methylene]-1H-1,2,3-triazolo[4,5-b]pyridinium 3-oxide hexafluorophosphate (HATU) (24 mg, 0.063 mmol) and DIEA (0.028 mL, 0.16 mmol). The mixture was stirred at 20° C. for 10 min. Then ammonium chloride (5.1 mg, 0.095 mmol) was added to the mixture and the mixture was stirred at 20° C. for 16 h. The mixture was then concentrated in vacuo, and the residue was purified by preparative TLC plate (SiO₂, DCM/MeOH=10/1) to afford tert-butyl 3-(2-((1-(tert-butoxycarbonyl)-3-carbamoylazetidin-3-yl)methoxy-6,8-difluoro-7-(3-(methoxymethoxy)naphthalen-1-yl)quinazolin-4-yl)-3,8-diazabicyclo[3.2.1]octane-8-carboxylate (Int-15c). MS (ESI): m, (M+H)⁺ 791.

### Step D: 3-(((4-(3.8-diazabicyclo[3.2.1]octan-3-yl)-6,8-difluoro-7-(3-hydroxynaphthalen-1-yl)quinazolin-2-yl)oxy)methyl)azetidine-3-carboxamide (Ex. 15)

To a mixture of tert-butyl 3-(2-((1-(tert-butoxycarbonyl)-3-carbamoylazetidin-3-yl)methoxy)-6,8-difluoro-7-(3-(methoxymethoxy) naphthalen-1-yl) quinazolin-4-y)-3,8-diazabicyclo[3.2.1]octane-8-carboxylate (Int-15c) (10 mg, 0.013 mmol) in CH₂Cl₂ (4.0 mL) was added TFA (1.0 mL, 13.0 mmol), and the mixture was stirred at 20° C. for 30 min. The mixture was then concentrated in vacuo. The resulting residue was purified by reverse phase preparative HPLC (MeCN/water with 0.1% TFA modifier) to afford 3-(((4-(3.8-diazabicyclo[3.2.1]octan-3-yl)-6,8-difluoro-7-(3-hydroxynaphthalen-1-yl)quinazolin-2-yl)oxy)methyl)azetidine-3-carboxamide (Ex. 15). MS (ESI): m/z (M+H)⁺ 547. ¹H NMR (400 MHz, CD₃OD) δ 7.78-7.69 (m, 2H), 7.42 (t, J=7.6 Hz, 1H), 7.34-7.27 (m, 2H), 7.25-7.19 (m, 1H), 7.11 (d, J=2.2 Hz, 1H), 4.67 (s, 2H), 4.46 (d, J=11.2 Hz, 2H), 4.25 (d, J=11.7 Hz, 4H), 3.84 (dd, J=6.7, 13.8 Hz, 2H), 2.20-2.14 (m, 4H), 1.29 (d, J=18.1 Hz. 2H).

| | | | | | SFC |
|---|---|---|---|---|---|
| | | | Compound | [M + H]⁺ | Purification |
| Ex. | Starting Material | Structure | Name | Found | Conditions |
| Ex. 117 | 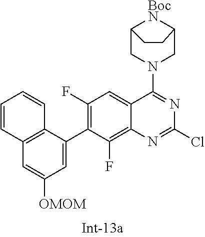 | 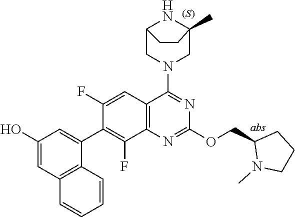 | 4-(6,8-difluoro- 4-((1S,5R)-1- methyl-3,8- diazabicyclo [3.2.1]octan- 3-yl)-2-(((R)-1- ethylpyrrolidin- 2-yl)methoxy) quinazolin-7- l)naphthalen- 2-ol | | |
| Ex.118 | 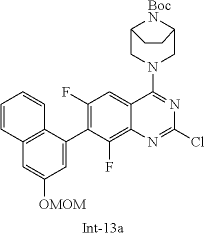 | 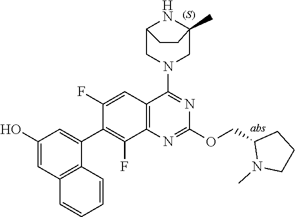 | 4-(6,8-difluoro- 4-((1S,5R)-1- methyl-3,8- diazabicyclo [3.2.1]octan- 3-yl)-2-(((S)-1- ethylpyrrolidin- 2-yl)methoxy) quinazolin-7- l)naphthalen- 2-ol | | |

### Example 16: (1R,5R,6S)-3-(6,8-difluoro-7-(3-hydroxynaphthalen-1-yl)-2-((1-(morpholinomethyl)cyclopropyl)methoxy)quinazolin-4-yl)-3,8-diazabicyclo[3.2.1]octan-6-ol (Ex. 16)

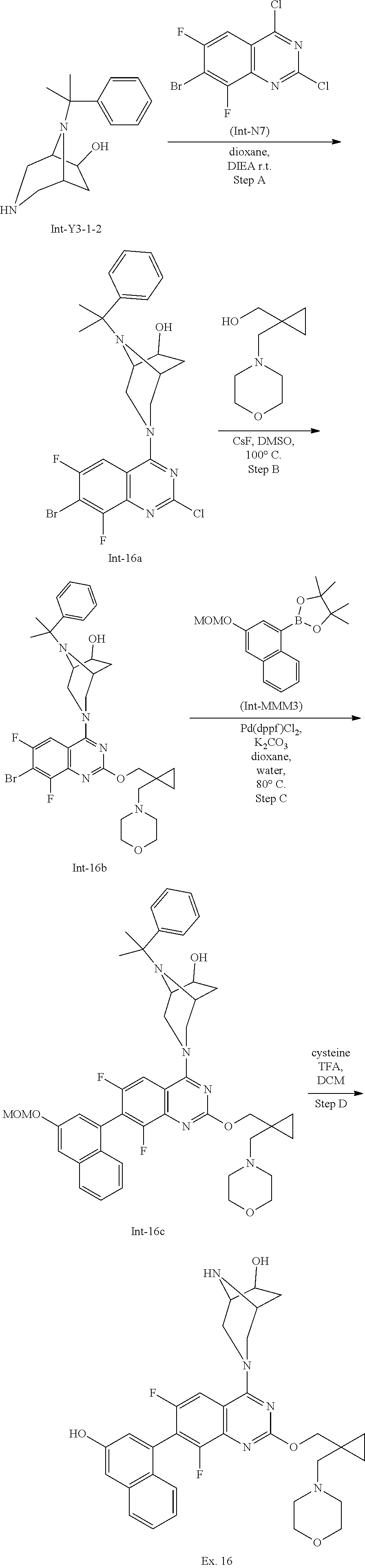

### Step A: (1R,5R,6S)-3-(7-bromo-2-chloro-6,8-difluoroquinazolin-4-yl)-8-(2-phenylpropan-2-yl)-3,8-diazabicyclo[3.2.1]octan-6-ol (Int-16a)

To a mixture of (1R,5R,6S)-8-(2-phenylpropan-2-yl)-3,8-diazabicyclo[3.2.1]octan-6-ol (Int-Y3-1-2) (0.18 g, 0.73 mmol) in dioxane (10 mL) were added DIEA (0.64 mL, 3.7 mmol) and 7-bromo-2,4-dichloro-6,8-difluoroquinazoline (Int-N₇, 0.23 g, 0.73 mmol). The mixture was stirred at 20° C. for 3 h. The mixture was then concentrated in vacuo. The resulting residue was purified by flash silica gel chromatography (0-20% ethyl acetate gradient in petroleum ether) to afford (1R,5R,6S)-3-(7-bromo-2-chloro-6,8-difluoroquinazolin-4-yl)-8-(2-phenylpropan-2-yl)-3,8-diazabicyclo[3.2.1]octan-6-ol (Int-16a). MS (ESI): m/z (M+H)Y 523, 525.

### Step B: (1R,5R,6S)-3-(7-bromo-6,8-difluoro-2-((1-(morpholinomethyl)cyclopropyl)methoxy)quinazolin-4-yl)-8-(2-phenylpropan-2-yl)-3,8-diazabicyclo[3.2.1]octan-6-ol (Int-16b)

To a stirred mixture of (1-(morpholinomethyl)cyclopropyl)methanol (0.12 g, 0.72 mmol) and (1R,5R,6S)-3-(7-bromo-2-chloro-6,8-difluoroquinazolin-4-yl)-8-(2-phenylpropan-2-yl)-3,8-diazabicyclo[3.2.1]octan-6-ol (Int-16a) (0.15 g, 0.29 mmol) in DMSO (5.0 mL) was added cesium fluoride (0.17 g, 1.1 mmol) at 20° C. under a nitrogen atmosphere. The reaction was then heated to 100° C. for 3 h. After 3 h, the mixture was allowed to cool to room temperature and then water (20 mL) was added. The resulting mixture was extracted with EtOAc (10 mL×3). The combined organic layers were dried over Na₂SO₄, filtered and concentrated in vacuo. The residue was purified by Prep-TLC (SiO₂, Pet, ether/EtOAc=1:1) to afford (1R,5R,6S)-3-(7-bromo-6,8-difluoro-2-((1-(morpholinomethyl)cyclopropyl)methoxy)quinazolin-4-yl)-8-(2-phenylpropan-2-yl)-3,8-diazabicyclo[3.2.1]octan-6-ol (Int-16b). MS (ESI): m/z (M+H)⁺ 658, 660.

### Step C: (1R,5R,6S)-3-(6,8-difluoro-7-(3-(methoxymethoxy)naphthalen-1-yl)-2-((1-(morpholinomethyl)cyclopropyl)methoxy)quinazolin-4-yl)-8-(2-phenylopropan-2-yl)-3,8-diazabicyclo[3.2.1]octan-6-ol (Int-16c)

To a stirred mixture of 2-(3-(methoxymethoxy)naphthalen-1-yl)-4,4,5,5-tetramethyl-1,3,2-dioxaborolane (Int-MMM3, 14 mg, 0.046 mmol), (1R,5R,6,S)-3-(7-bromo-6,8-difluoro-2-((1-(morpholinomethyl)cyclopropyl)methoxy)quinazolin-4-yl)-8-(2-phenylpropan-2-yl)-3,8-diazabicyclo[3.2.1]octan-6-ol (Int-16b) (30 mg, 0.046 mmol) in dioxane (1.5 mL) and water (0.30 mL) were added PdCl₂(dppf) (6.7 mg, 9.1 μmol) and Na₂CO₃ (12 mg, 0.11 mmol) at 20° C. under a N₂ atmosphere. The resulting mixture was heated to 80° C. for 2 h. After 2 h, the mixture was allowed to cool to room temperature and then concentrated under reduced pressure. The residue was purified by prep-TLC (SiO₂, Pet, ether/EtOAc=1:1) to afford (1R,5R,6S)-3-(6,8-difluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octan-6-ol (Int-16c). MS (ESI): m/z (M+H)⁺ 766.

### Step D: (1R,5R,6S)-3-(6,8-difluoro-7-(3-hydroxynaphthalen-1-yl)-2-((1-(morpholinomethyl)cyclopropyl)methoxy)quinazolin-4-yl)-3,8-diazabicyclo[3.2.1]octan-6-ol (Ex. 16)

To a mixture of (1R,5R,6S)-3-(6,8-difluoro-7-(3-(methoxymethoxy)naphthalen-1-yl)-2-((1-(morpholinomethyl)cyclopropyl)methoxy)quinazolin-4-yl)-8-(2-phenylpropan-2-yl)-3,8-diazabicyclo[3.2.1]octan-6-ol (Int-16c) (20 mg, 0.026 mmol) were added CH₂Cl₂ (4.0 mL), cysteine (6.3 mg, 0.052 mmol) and TFA (1.0 mL) at 20° C. The mixture was stirred at 20° C. for 3 h. The mixture was then concentrated under reduced pressure and the resulting residue was purified by reverse phase Prep-HPLC (MeCN/water with 0.1% ammonium hydroxide and 10 mM ammonium bicarbonate modifier) to afford (1R,5R,6S)-3-(6,8-difluoro-7-(3-hydroxynaphthalen-1-yl)-2-((1-(morpholinomethyl)cyclopropyl) methoxy)quinazolin-4-yl)-3,8-diazabicyclo[3.2.1]octan-6-ol (Ex. 16). MS (ESI): m/z (M+H)⁺ 604. ¹H NMR (400 MHz, CD₃OD) δ 7.75 (d, J=8.4 Hz, 1H), 7.62 (br d, J=9.2 Hz, 1H), 7.42 (t, J=7.6 Hz, 1H), 7.34 (br d, J=8.0 Hz, 1H), 7.30-7.18 (m, 2H), 7.11 (d, J=2.0 Hz, 1H), 4.58 (br s, 1H), 4.50-4.24 (m, 4H), 3.84-3.75 (m, 1H), 3.72-3.45 (m, 6H), 3.41 (s, 1H), 2.60-2.37 (m, 6H), 2.25 (dd, J=6.8, 14.0 Hz, 1H), 1.74 (dd, J=6.8, 14.0 Hz, 1H), 0.75-0.66 (m, 2H), 0.53-0.43 (m, 2H).

The procedure used to prepare the example below was similar to that above, using Int-Y3-1-1 as the starting material.

| | | | [M + H]⁺ |
|---|---|---|---|
| Ex | Structure | Compound Name | Found |
| Ex 17 | 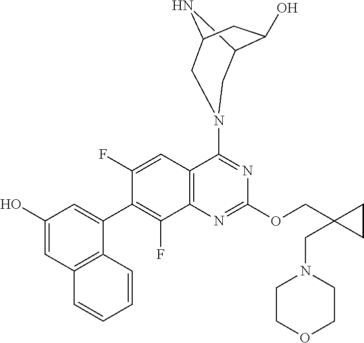 [SMILES: Oc1cc(-c2c(F)cc3c(N4CC5CC(O)C(C4)N5)nc(OCC4(CN5CCOCC5)CC4)nc3c2F)c2ccccc2c1] | (1S,5S,6R)-3-(6,8-difluoro- 7-(3-hydroxynaphthalen-1- yl)-2-((1- (morpholinomethyl)cyclo- propyl)methoxy)quinazolin- 4-yl)-3,8- diazabicyclo[3.2.1]octan-6- ol | 604 |

Example 18: (1R,5R,6R)-3-(6-chloro-2-((1

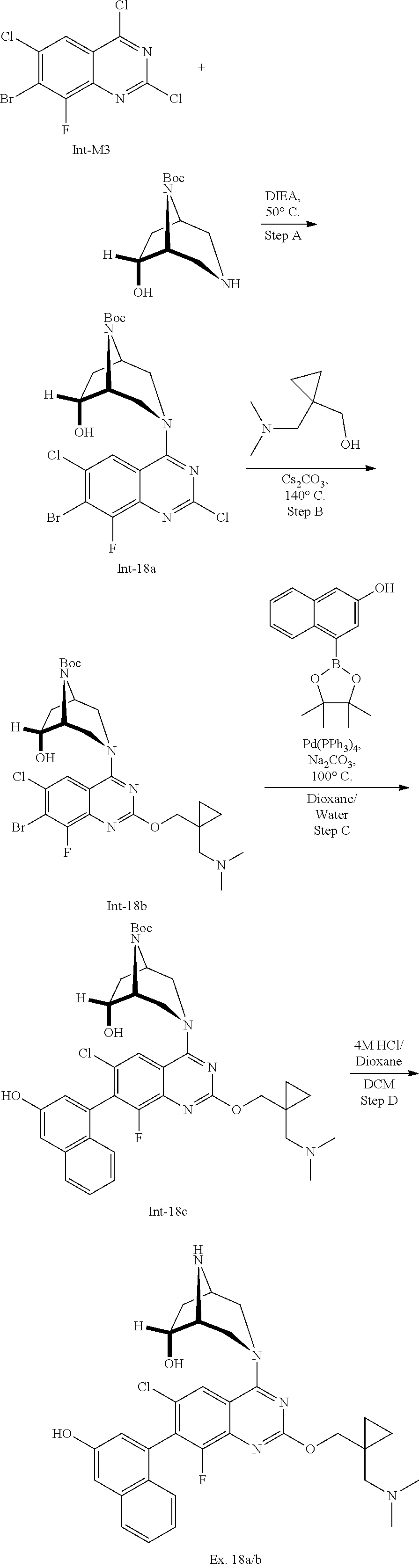

### Step A: tert-butyl (1R,5R,6R)-3-(7-bromo-2,6-dichloro-8-fluoroquinazolin-4-yl-6-hydroxy-3,8-diazabicyclo[3.2.1]octane-8-carboxylate (Int-18a)

To a mixture of 7-bromo-2,4,6-trichloro-8-fluoroquinazoline (Int-M3) (85 mg, 0.26 mmol) and DIEA (0.23 mL, 1.3 mmol) in dioxane (1,3 mL) was added tert-butyl (1R,5R,6R)-6-hydroxy-3,8-diazabicyclo[3.2.1]octane-8-carboxylate (58 mg, 0.26 mmol). The reaction mixture was heated to 50° C. for 20 mins. The mixture was then allowed to cool to room temperature and partitioned between water and DCM. The organic layer was separated, dried over magnesium sulfate, filtered and then concentrated under reduced pressure afford tert-butyl (1R,5R,6R)-3-(7-bromo-2,6-dichloro-8-fluoroquinazolin-4-yl)-6-hydroxy-3,8-diazabicyclo[3.2.1]octane-8-carboxylate (Int-18a). The resulting product was carried on to the next transformation without further purification. MS (ESI): m/z (M+H)⁺ 521, 523.

### Step B: tert-butyl (1R,5R,6R)-3-(7-bromo-6-chloro-2-((1-((dimethylamino)methyl)cyclopropyl)methoxy)-8-fluoroquinazolin-4-yl)-6-hydroxy-3,8-diazabicyclo[3.2.1]octane-8-carboxylate (Int-18b)

To a mixture of tert-butyl (1R,5R,6R)-3-(7-bromo-2,6-dichloro-8-fluoroquinazolin-4-yl)-6-hydroxy-3,8-diazabicyclo[3.2.1]octane-8-carboxylate (Int-18a) (0.13 g, 0.26 mmol) in dioxane (2.6 mL) was added cesium carbonate (0.25 g, 0.77 mmol). The reaction mixture was heated to 140° C. for 3 h. After 3 h, the resulting mixture was allowed to cool to room temperature and partitioned between water and chloroform/IPA (3:1). The organic layer was separated and concentrated under reduced pressure. The crude mixture was purified by column chromatography on silica (0-10% (7 N ammonia in methanol) in DCM gradient) to afford tert-butyl (1R,5R,6R)-3-(7-bromo-6-chloro-2-((1-((dimethylamino)methyl)cyclopropyl)methoxy)-8-fluoroquinazolin-4-yl)-6-hydroxy-3,8-diazabicyclo[3.2.1]octane-8-carboxylate (Int-18b). MS (ESI): m/z (M+H)⁺ 614, 616.

### Step C: tert-butyl (1R,5R,6R)-3-(6-chloro-2-((1-((dimethylamino)methyl)cyclopropyl)methoxy)-8-fluoro-7-(3-hydroxynaphthalen-1-yl)quinazolin-4-yl)-6-hydroxy-3,8-diazabicyclo[3.2.1]octane-8-carboxylate (Int-18c

The mixture of tert-butyl (1R,5R,6R)-3-(7-bromo-6-chloro-2-((1-((dimethylamino)methyl)cyclopropyl)methoxy)-8-fluoroquinazolin-4-yl)-6-hydroxy-3,8-diazabicyclo[3.2.1]octane-8-carboxylate (Int-18b) (67 mg, 0.11 mmol) and 4-(4,4,5,5-tetramethyl-1,3,2-dioxaborolan-2-yl)naphthalen-2-ol (0.12 mg, 0.44 mmol), sodium carbonate (35 mg, 0.33 mmol) in 1.4-dioxane (0.870 mL) and water (0.22 mL) was heated to 100° C. for 16 h. After 16 h, the resulting mixture was allowed to cool to room temperature and partitioned between water and chloroform/IPA (3:1). The organic layer was separated and concentrated under reduced pressure to afford tert-butyl (1R,5R,6R)-3-(6-chloro-2-((l-((dimethylamino)methyl)cyclopropyl)methoxy)-8-fluoro-7-(3-hydroxynaphthalen-1-yl)quinazolin-4-yl)-6-hydroxy-3,8-diazabicyclo[3.2.1]octane-8-carboxylate (Int-18c) which was used in future transformations without further purification. MS (ESI): m/z (M+H)⁺ 678.

### Step D: (0R,5R,6R)-3-(6-chloro-2-((1-((dimethylamino)methyl)cyclopropyl)methoxy)-8-fluoro-7-(3-hydroxynaphthalen-1-yl)quinazolin-4-yl)-3,8-diazabicyclo[3.2.1]octan-6-ol (Ex. 18a/b)

DCM (0.54 mL) was added to a 20-mL scintillation vial containing tert-butyl (1R,5R, 6R)-3-(6-chloro-2-((1-((dimethylamino)methyl)cyclopropyl)methoxy)-8-fluoro-7-(3-hydroxynaphthalen-1-yl)quinazolin-4-yl)-6-hydroxy-3,8-diazabicyclo[3.2.1]octane-8-carboxylate (Int-18c) (73 mg, 0.11 mmol) and a stir bar. Hydrochloric acid (4.0 M in dioxane, 0.27 mL, 1.1 mmol) was added. The reaction mixture was stirred at room temperature for 2 h. After 2 h, diethyl ether was added to the resulting mixture. The resulting solids were filtered and washed with ether. The solid was purified by reverse phase HPLC (MeCN/water with 0.1% ammonium hydroxide modifier) to afford (1R,5R,6R)-3-(6-chloro-2-((1-((dimethylamino)methyl)cyclopropyl)methoxy)-8-fluoro-7-(3-hydroxynaphthalen-1-yl)quinazolin-4-yl)-3,8-diazabicyclo[3.2.1]octan-6-ol (Ex. 18a, Peak 1). MS (ESI): m/z (M+H)⁺ 578. ¹H NMR (600 MHz, DMSO-d₆) δ 10.05 (s, 1H), 8.54 (s, 1H), 7.82 (d, J=8.3 Hz, 1H), 7.45 (t, J=7.7 Hz, 1H). 7.30 (d, J=2.1 Hz, 1H), 7.27-7.21 (m, 1H), 7.16 (d, J=8.3 Hz, 1H), 7.07 (d, J=2,3 Hz, 1H), 6.55 (s, 1H), 5.51 (s, 1H), 4.524.49 (m, 1H), 4.38-4.35 (m, 2H), 4.28-4.22 (m, 2H), 3.84-3.80 (m, 1H), 3.69-3.63 (m, 1H), 3.62-3.56 (m, 1H), 3.50-3.45 (m, 1H), 2.50-2.36 (m, 6H), 2.35-2.22 (m, 2H), 1.67-1.60 (m, 1H), 1.27-1.22 (m, 1H), 0.78-0.69 (m, 2H), 0.60-0.51 (m, 2H). (1R,5R, 6R)-3-(6-chloro-2-((1-((dimethylamino)methyl)cyclopropyl)methoxy)-8-fluoro-7-(3-hydroxynaphthalen-1-yl)quinazolin-4-yl)-3,8-diazabicyclo[3.2.1]octan-6-ol (Ex. 18b, Peak 2). MS (ESI): m/z (M+H)⁺ 578. ¹H NMR (600 MHz, DMSO-d₆) δ 10.03 (s, 1H), 8.48 (s, 1H), 7.82 (d, J=8.3 Hz, 1H), 7.46-7.43 (m, 1H), 7.29 (d. J=2.1 Hz. 1H), 7.24-7.21 (m, 1H), 7.20-7.16 (m, 1H), 7.07 (d, J=2,3 Hz, 1H), 6.54 (s, 1H), 5.33 (s, 1H), 4.51-4.44 (m, 1H), 4.43-4.35 (m, 1H), 4.35-4.28 (m, 2H), 4.27-4.19 (m, 2H), 3.73-3.68 (m, 1H), 3.68-3.56 (m, 2H), 3.46-3.43 (m, 1H), 2.37 (d, J=32.8 Hz, 6H), 2.29-2.22 (m, 2H), 1.60-1.52 (m, 1H), 0.75-0.67 (m, 2H), 0.57-0.47 (m, 2H).

The procedure used to prepare the examples below was similar to that above, using tert-butyl (1S,5S,6S)-6-hydroxy-3,8-diazabicyclo[3.2.1]octane-8-carboxylate (Int-RS1-1) as the starting material.

| | | | [M + H]⁺ |
|---|---|---|---|
| Ex | Structure | Compound Name | Found |
| Ex 19a | 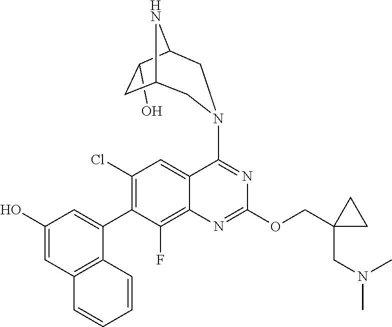 [SMILES: CN(C)CC1(COc2nc(N3CC4CC(O)C(C3)N4)c3cc(Cl)c(-c4cc(O)cc5ccccc45)c(F)c3n2)CC1] | (1S,5S,6S)-3-(6-chloro-2-((1- ((dimethylamino)methyl) cyclopropyl)methoxy)-8- fluoro-7-(3-hydroxynaphthalen- 1-yl)quinazolin-4- yl)-3,8-diazabicyclo [3.2.1]octan-6-ol | 578 |
| | Peak 1 | | |
| Ex 19b | 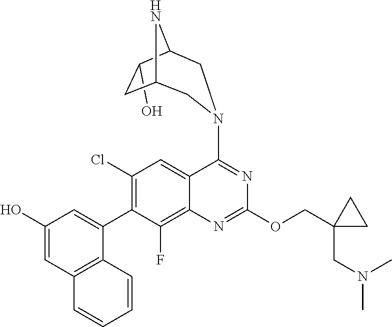 [SMILES: CN(C)CC1(COc2nc(N3CC4CC(O)C(C3)N4)c3cc(Cl)c(-c4cc(O)cc5ccccc45)c(F)c3n2)CC1] | (1S,5S,6S)-3-(6-chloro-2-((1- ((dimethylamino)methyl) cyclopropyl)methoxy)-8- fluoro-7-(3-hydroxynaphthalen- 1-yl)quinazolin-4- yl)-3,8-diazabicyclo [3.2.1]octan-6-ol | 578 |
| | Peak 2 | | |
| Ex.119 | 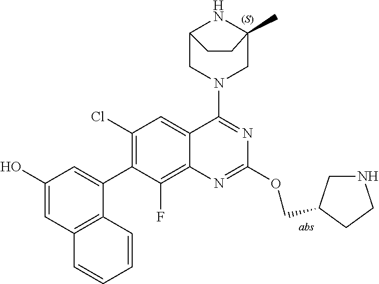 | 4-(6-chloro-8-fluoro-4- ((1S,5R)-1-methyl-3,8- diazabicyclo[3.2.1]octan-3- yl)-2-(((S)-pyrrolidin-3-yl) methoxy)quinazolin-7- yl)naphthalen-2-ol, TFA | 548 |
| | mix. of atropisomers | | |
| Ex.120 | 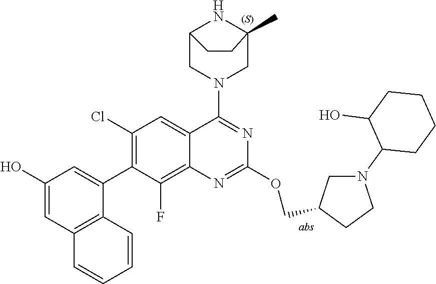 | 4-(6-chloro-8-fluoro- 2-(((3S)-1-(2-hydroxy- cyclohexyl)pyrrolidin-3- yl)methoxy)-4-((1S,5R)-1- methyl-3,8-diazabicyclo [3.2.1]octan-3- yl)quinazolin-7- yl)naphthalen-2-ol, TFA | 646 |
| | mix. of atropisomers | | |
| Ex.121 | 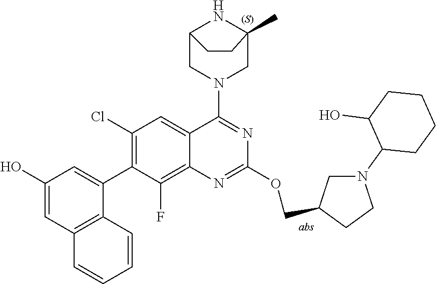 | 4-(6-chloro-8-fluoro-2-(((3R)- 1-(2-hydroxycyclohexyl) pyrrolidin-3-yl)methoxy)-4- ((1S,5R)-1-methyl-3,8- diazabicyclo[3.2.1]octan-3-yl) quinazolin-7-yl)naphthalen- 2-ol, TFA | 646 |
| | mix. of atropisomers | | |
| Ex.122 | 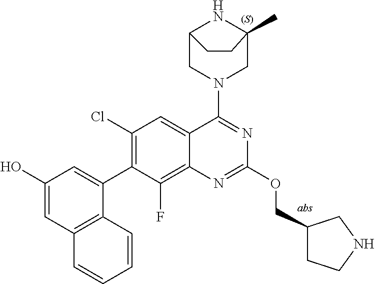 | 4-(6-chloro-8-fluoro-4- ((1S,5R)-1-methyl-3,8- diazabicyclo[3.2.1]octan-3- yl)-2-(((R)-pyrrolidin-3-yl) methoxy)quinazolin-7- yl)naphthalen-2-ol, TFA | 548 |
| | mix. of atropisomers | | |

### Example 20: 4-(2-((1-((dimethylamino)methyl)cyclopropyl)methoxy)-6,8-difluoro-4-((1R5S)--1-methyl-3,8-diazabicyclo[3.2.1]octan-3-yl)quinazolin-7-yl)naphthalen-2-ol (Ex. 20)

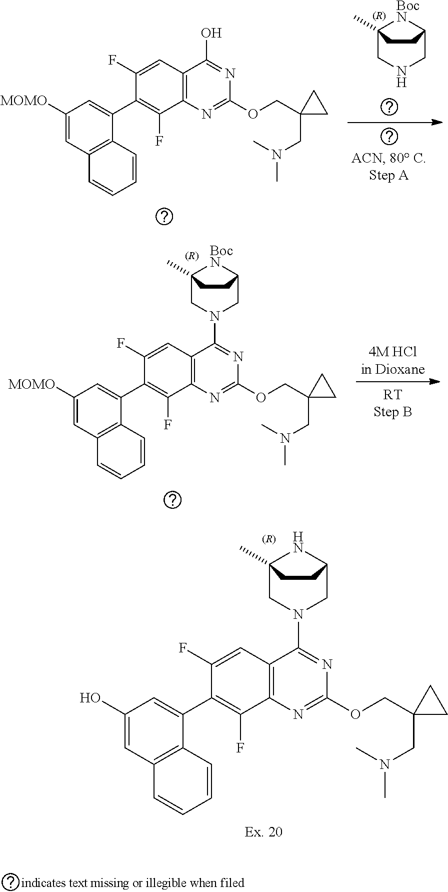

### Step A: tert-butyl (1R,5S)-3-(2-((1-((dimethylamino)methyl)cyclopropyl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (Int-20a)

To a mixture of 2-((1-((dimethylamino)methyl)cyclopropyl)methoxy)-6,8-difluoro-7-(3-(methoxymethoxy)naphthalen-1-yl)quinazolin-4-ol (Int-W4) (42 mg, 0.085 mmol) in acetonitrile (1.0 mL) were added tert-butyl (1R,5S)-1-methyl-3,8-diazabicyclo[3.2.1]octane-8-carboxylate (Int-ill-1) (29 mg, 0.13 mmol), BOP (49 mg, 0.11 mmol) and DBU (39 p L, 0.25 mmol). The resulting mixture was heated to 80° C. for 16 h. After 16 h, the reaction mixture was cooled to 24° C., quenched with water and extracted with EtOAc. The organic layer was washed with saturated brine solution, dried over magnesium sulfate, filtered and the filtrate was concentrated under reduced pressure. The resulting residue was purified by silica gel column chromatography (0-35% EtOAc gradient in hexanes) to afford tert-butyl (1R,5S)-3-(2-((1-((dimethylamino)methyl)cyclopropyl)methoxy)-6,8-difluoro-7-(3-(methoxymethoxy)naphthalen-1-yl)quinazolin-4-yl)-1-methyl-3,8-diazabicyclo[3.2.1]octane-8-carboxylate (Int-20a). MS (ESI): m/z (M+H)⁺ 704.

### Step B: 4-(2-((14(dimethylamino)methyl)cyclopropyl)methoxy)-6,8-difluoro-4-((1R,5S)-1-methyl-3,8-diazabicyclo[3.2.1]octan-3-yl)quinazolin-7-yl)naphthalen-2-ol (Ex. 20)

To a mixture of tert-butyl (1R,5S)-3-(2-((1-((dimethylamino)methyl)cyclopropyl)methyl)-6,8-difluoro-7-(3-(methoxymethoxy)naphthalen-1-yl)quinazolin-4-yl)-1-methyl-3,8-diazabicyclo[3.2.1]octane-8-carboxylate (Int-20a) in DCM (1.0 mL) was added HCl (4.0 M in dioxane, 0.17 mL, 0.68 mmol). The reaction mixture was stirred at 24° C. for 3 h. The reaction mixture was then diluted with diethyl ether (5.0 mL) and stirred for 5 mins. The organic layers were decanted and the solid material was concentrated in vacuo. The residue was purified by mass triggered reverse phase HPLC (MeCN/water with 0.05% NH₄HCO₃ modifier) to afford 4-(2-((1-((dimethylamino)methyl)cyclopropyl)methoxy)-6,8-difluoro-4-((1R,5S)-1-methyl-3,8-diazabicyclo[3.2.1]octan-3-yl)quinazolin-7-yl)naphthalen-2-ol (Ex. 20). MS (ESI): m/z (M+H)⁺ 560. ¹H NMR (5RX MHz, DMSO-d₆) δ 7.82 (d, J=8.2 Hz, 1H), 7.71 (d, J=10.3 Hz, 1H), 7.46 (t, J=7.5 Hz. 1H), 7.34 (d, J=3.9 Hz, 2H), 7.25 (t, J=7.6 Hz, 1H), 7.15 (s, 1H), 4.33-4.19 (m, 4H), 3.25 (d, J=3.9 Hz, 2H), 2.23 (s, 2H), 2.16 (s, 6H), 1.86-1.56 (m, 4H), 1.33 (dt, J=15.2, 8.8 Hz, 1H), 1.24 (s, 3H), 0.68-0.57 (m, 2H), 0.45-0.33 (m, 2H).

Compounds in the table below were synthesized via a similar route as Ex. 20 using the amine starting materials indicated in the table.

| | | | | [M + | SFC |
|---|---|---|---|---|---|
| | | | | H]⁺ | Purification |
| Ex. | Starting Material | Structure | Compound Name | Found | Conditions |
| Ex 21 | 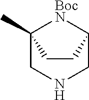 [SMILES: CC(C)(C)OC(=O)N1[C@@H]2CC[C@@]1(C)CNC2] Int-II1-2 | 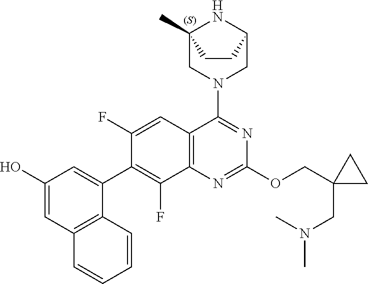 | 4-(2-((1-((dimethyl- amino)methyl)cyclo- propyl)methoxy)- 6,8-difluoro-4- ((1S,5R)-1-methyl-3,8- diazabicy- clo[3.2.1]octan- 3-yl)quinazolin-7- yl)naphthalen-2-ol | 560 | N/A |
| | | Peak 2 | | | |
| Ex. 22a | 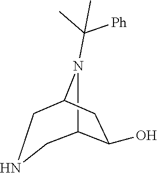 [SMILES: CC(C)(c1ccccc1)N1C2CNCC1C(O)C2] Int-Y3-1-1 | 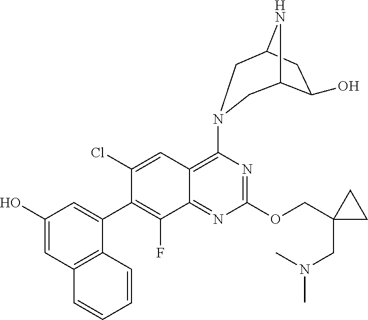 [SMILES: CN(C)CC1(COc2nc(N3CC4CC(O)C(C3)N4)c3cc(Cl)c(-c4cc(O)cc5ccccc45)c(F)c3n2)CC1] | 3-(6-chloro-2- ((1-((dimethyl- amino)methyl)cyclo- propy)methoxy)- 8-fluoro-7-(3-hydroxy- naphthalen-1- yl)quinazolin-4-yl)-3,8- diazabicyclo[3.2.1]oc tan-6-ol | 578 | Column A: Condition: 0.1% NH₄OH in EtOH |
| | | Peak 1 | | | |
| Ex. 22b | 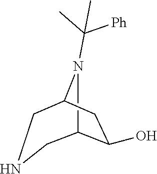 [SMILES: CC(C)(c1ccccc1)N1C2CNCC1C(O)C2] Int-Y3-1-1 | 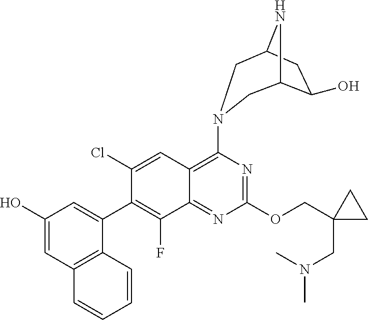 [SMILES: CN(C)CC1(COc2nc(N3CC4CC(O)C(C3)N4)c3cc(Cl)c(-c4cc(O)cc5ccccc45)c(F)c3n2)CC1] | 3-(6-chloro-2-((1- ((dimethylamino)meth- yl)cyclo- propyl)methoxy)- 8-fluoro-7-(3-hydroxy- naphthalen-1- yl)quinazolin-4-yl)-3,8- diazabicyclo[3.2.1]oc- tan-6-ol | 578 | |
| | | Peak 2 | | | |
| Ex 23 | 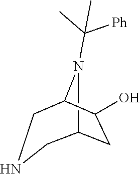 [SMILES: CC(C)(c1ccccc1)N1C2CNCC1C(O)C2] Int-Y3-1-2 | 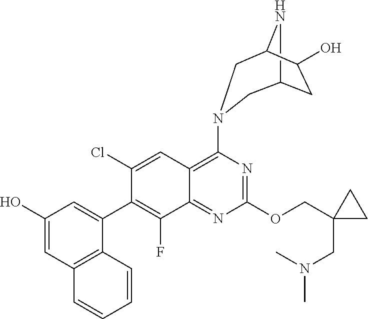 [SMILES: CN(C)CC1(COc2nc(N3CC4CC(O)C(C3)N4)c3cc(Cl)c(-c4cc(O)cc5ccccc45)c(F)c3n2)CC1] | 3-(6-chloro-2-((1- ((dimethyalmino)meth- yl)cyclopropyl)meth- oxy)-8-fluoro-7-(3- hydroxynaphthalen-1- yl)quinazolin-4-yl)-3,8- diazabicyclo[3.2.1]oc- tan-6-ol | 578 | Column K; Condition: 0.1% NH₄OH in EtOH |
| | | Peak 1 | | | |
| Ex 24 | 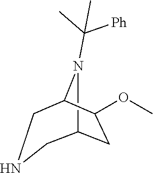 [SMILES: COC1CC2CNCC1N2C(C)(C)c1ccccc1] Int-AA2 | 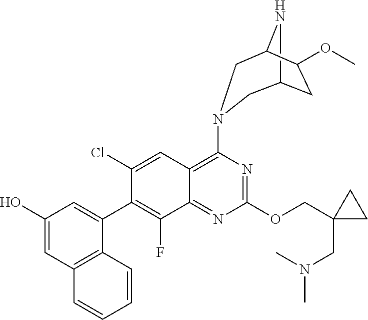 [SMILES: COC1CC2CN(c3nc(OCC4(CN(C)C)CC4)nc4c(F)c(-c5cc(O)cc6ccccc56)c(Cl)cc34)CC1N2] | 4-(2-((1- ((dimethylamino)meth- yl)cyclopropyl)meth- oxy)-6,8-difluoro-4-(6- methoxy-3,8-di- azabicyclo[3.2.1]octan- 3-yl)quinazolin-7- yl)naphthalen-2-olR,R, | 576 | N/A |
| Ex 25 | 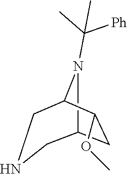 [SMILES: COC1CC2CNCC1N2C(C)(C)c1ccccc1] Int-Z2 | 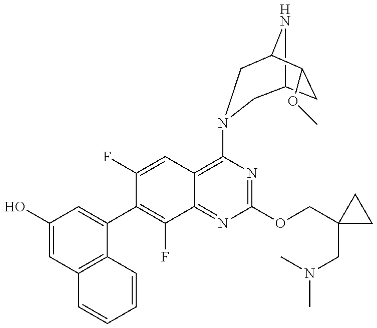 [SMILES: COC1CC2CN(c3nc(OCC4(CN(C)C)CC4)nc4c(F)c(-c5cc(O)cc6ccccc56)c(F)cc34)CC1N2] | 4-(2-((1-((dimethyl- amino)methyl)cyclo- propyl)methoxy)-6,8- difluoro-4-(6- methoxy-3,8- diazabicyclo[3.2.1]oc- tan-3-yl)quinazolin- 7-yl)naphthalen-2-ol | 576 | N/A |
| Ex 26 | 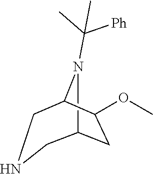 [SMILES: COC1CC2CNCC1N2C(C)(C)c1ccccc1] Int-AA2 | 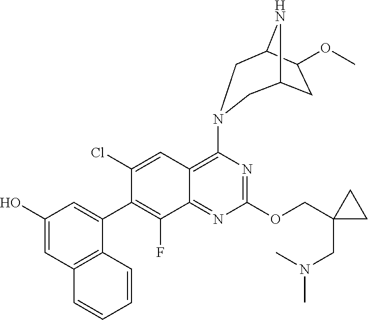 [SMILES: COC1CC2CN(c3nc(OCC4(CN(C)C)CC4)nc4c(F)c(-c5cc(O)cc6ccccc56)c(Cl)cc34)CC1N2] | 4-(6-chloro-2-((1- ((dimethylamino)meth- yl)cyclo- propyl)methoxy)- 8-fluoro-4-(R,R,6- methoxy-3,8- diazabi- cyclo[3.2.1]octan- 3-yl)quinazolin-7- yl)naphthalen-2-ol | 592 | Column C; Condition: DCM- MeOH |
| | | Peak 2, SFC at penultimate step | | | |
| Ex 27 | 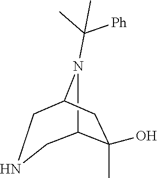 [SMILES: CC1(O)CC2CNCC1N2C(C)(C)c1ccccc1] Int-DD2-1 | 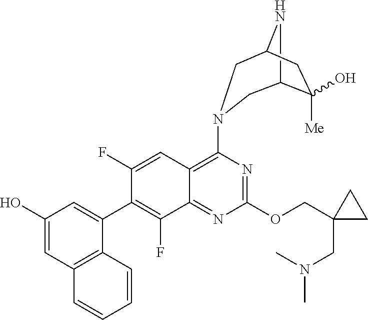 [SMILES: CN(C)CC1(COc2nc(N3CC4CC(C)(O)C(C3)N4)c3cc(F)c(-c4cc(O)cc5ccccc45)c(F)c3n2)CC1] | 3-(2-((1- ((dimethylamino)meth- yl)cyclopro- pyl)methoxy)- 6,8-difluoro-7-(3- hydroxynaphthalen- 1-yl)quinazolin-4-yl)- 6-methyl-3,8- diazabicyclo [3.2.1]octan-6-ol | 576 | N/A |
| Ex 28 | 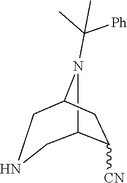 [SMILES: CC(C)(c1ccccc1)N1C2CNCC1C(C#N)C2] Int-BB4-1 | 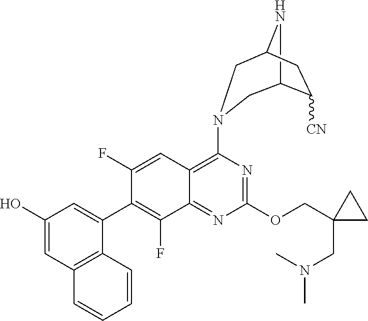 [SMILES: CN(C)CC1(COc2nc(N3CC4CC(C#N)C(C3)N4)c3cc(F)c(-c4cc(O)cc5ccccc45)c(F)c3n2)CC1] | 3-(2-((1- ((dimethylamino)meth- yl)cyclopro- pyl)methoxy)- 6,8-difluoro-7-(3- hydroxynaphthalen-1- yl)quinazolin-4-yl)-3,8- diazabicyclo[3.2.1]oc- tane-6-carbontrile | 571 | N/A |
| Ex 29 | 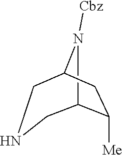 [SMILES: CC1CC2CNCC1N2C(=O)OCc1ccccc1] Int-CC5-2 | 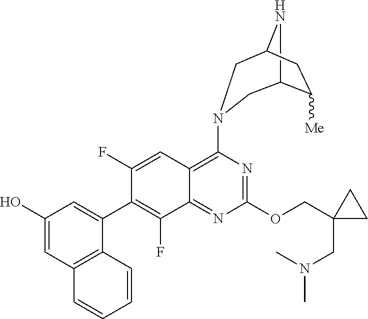 [SMILES: CC1CC2CN(c3nc(OCC4(CN(C)C)CC4)nc4c(F)c(-c5cc(O)cc6ccccc56)c(F)cc34)CC1N2] | 4-(2-((1-((dimethyl- amino) methyl)cyclopro- pyl)methoxy)-6,8- difluoro-4-(6- methyl-3,8- diazabicyclo[3.2.1]oc- tan-3-yl)quinazolin-7- yl)naphthalen-2-ol | 560 | N/A |
| Ex 30 | 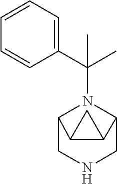 [SMILES: CC(C)(c1ccccc1)N1C2CNCC1C1CC12] Int-GG2 | 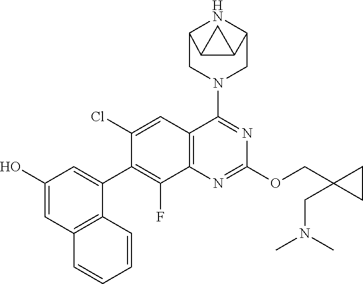 [SMILES: CN(C)CC1(COc2nc(N3CC4NC(C3)C3CC43)c3cc(Cl)c(-c4cc(O)cc5ccccc45)c(F)c3n2)CC1] | 4-(6-chloro-2-((1- ((dimethylamino)meth- yl)cyclopropyl)meth- oxy)-8-fluoro- 4-(7,9-diazatri- cyclo[3.3.1.02,4]nonan- 7-yl)quinazolin-7- yl)naphthalen-2-ol | 574 | N/A |
| Ex 31 | 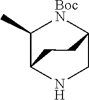 [SMILES: C[C@@H]1[C@@H]2CC[C@@H](CN2)N1C(=O)OC(C)(C)C] Int-MM2 | 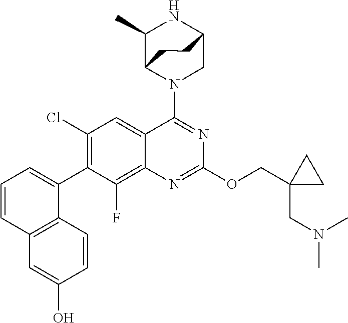 [SMILES: C[C@H]1N[C@H]2CC[C@@H]1N(c1nc(OCC3(CN(C)C)CC3)nc3c(F)c(-c4cccc5cc(O)ccc45)c(Cl)cc13)C2] | 4-(6-chloro-2-((1- ((dimethylamino)meth- yl)cyclopropyl)meth- oxy)-8-fluoro-4- ((1S,4S,6R)-6- methyl-2,5- diazabicyclo [2.2.2]octan-2- yl)quinazolin- 7-yl)naphthalen-2-ol | 576 | N/A |
| Ex. 32 | 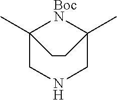 Int-HH4 | 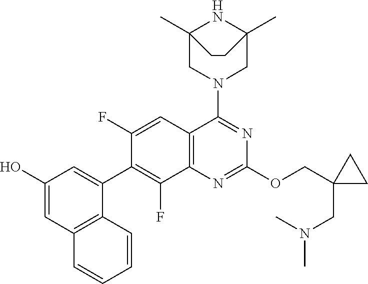 [SMILES: CN(C)CC1(COc2nc(N3CC4(C)CCC(C)(C3)N4)c3cc(F)c(-c4cc(O)cc5ccccc45)c(F)c3n2)CC1] | 4-(4-(1,5-dimethyl-3,8- diazabicy- clo[3.2.1]octan- 3-yl)-2-((1-((dimethyl- amino)methyl)cyclo- propyl)methoxy)-6,8- difluoroquinazolin-7- yl)naphthalen-2-ol | 574 | N/A |
| Ex 33 | 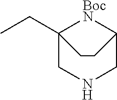 [SMILES: CCC12CCC(CNC1)N2C(=O)OC(C)(C)C] Int-332 | 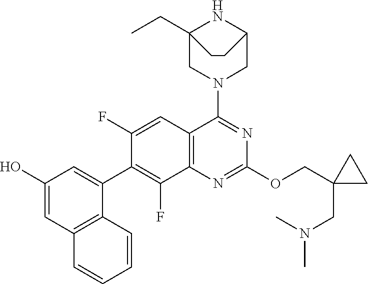 [SMILES: CCC12CCC(CN(c3nc(OCC4(CN(C)C)CC4)nc4c(F)c(-c5cc(O)cc6ccccc56)c(F)cc34)C1)N2] | 4-(2-((1- ((dimethylamino)meth- yl)cyclopro- pyl)methoxy)-4-(1- ethyl-3,8-diazabicy- clo[3.2.1]octan-3-yl)- 6,8-difluoroquinazolin- 7-yl)naphthalen-2-ol | 574 | N/A |
| Ex 34 | 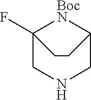 [SMILES: CC(C)(C)OC(=O)N1C2CCC1(F)CNC2] Int-KK2 | 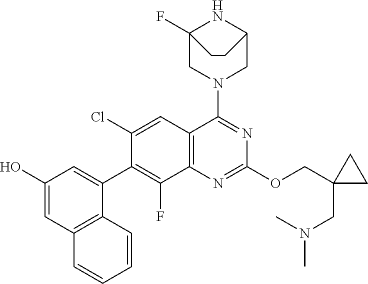 [SMILES: CN(C)CC1(COc2nc(N3CC4CCC(F)(C3)N4)c3cc(Cl)c(-c4cc(O)cc5ccccc45)c(F)c3n2)CC1] | 4-(6-chloro-2-((1- ((dimethylamino)meth- yl)cyclopropyl)meth- oxy)-8-fluoro-4- (1-fluoro-3,8-diaza- bicyclo[3.2.1]octan- 3-yl)quinaozlin-7- yl)naphthalen-2-ol | 580 | N/A |
| Ex 35 | 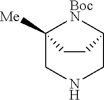 [SMILES: CC(C)(C)OC(=O)N1[C@@H]2CC[C@@]1(C)CNC2] Int-II1-2 | 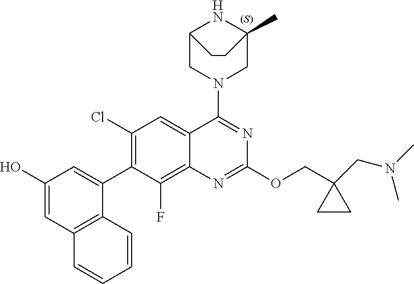 | 4-(6-chloro-2-((1- ((dimethylamino)meth- yl)cyclopro- pyl)methoxy)- 8-fluoro-4-((1S,5R)-1- methyl-3,8-diaza- bicyclo[3.2.1]octan- 3-yl)quinazolin-7- yl)naphthalen-2-ol | 576 | N/A |
| Ex 36 | 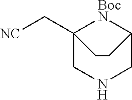 [SMILES: CC(C)(C)OC(=O)N1C2CCC1(CC#N)CNC2] Int-PP4-1 | 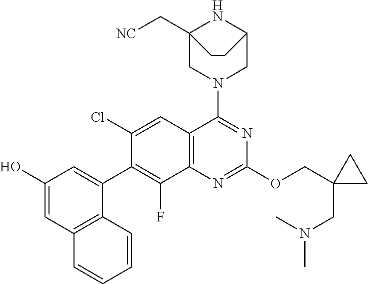 [SMILES: CN(C)CC1(COc2nc(N3CC4CCC(CC#N)(C3)N4)c3cc(Cl)c(-c4cc(O)cc5ccccc45)c(F)c3n2)CC1] | 2-(3-(6-chloro-2-((1- ((dimethylamino)meth- yl)cyclopro- pyl)methoxy)- 8-fluoro-7-(3-hydroxy- naphthalen-1-yl) quinazolin-4-yl)-3,8- diazabicyclo[3.2.1]oc- tan-1-yl)acetonitrile | 601 | N/A |
| Ex 37 | 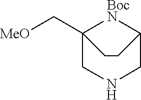 [SMILES: COCC12CCC(CNC1)N2C(=O)OC(C)(C)C] Int-QQ4-1 | 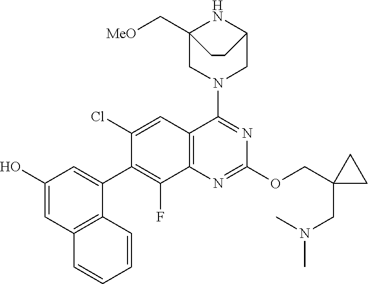 [SMILES: COCC12CCC(CN(c3nc(OCC4(CN(C)C)CC4)nc4c(F)c(-c5cc(O)cc6ccccc56)c(Cl)cc34)C1)N2] | 4-(6-chloro-2-((1- ((dimethylamino)meth- yl)cyclopro- pyl)methoxy)-8- fluoro-4-(1-(methoxy- methyl)-3,8- diazabicyclo[3.2.1]oc- tan-3-yl)quinazolin-7- yl)naphthalen-2-ol | 606 | N/A |
| Ex 38a | 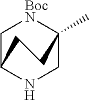 [SMILES: CC(C)(C)OC(=O)N1C[C@@H]2CC[C@]1(C)CN2] Int-LL2 | 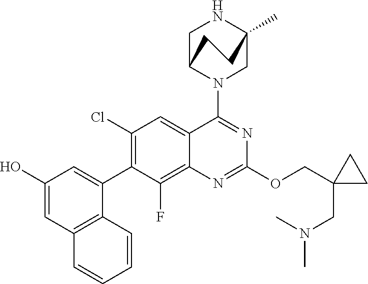 [SMILES: CN(C)CC1(COc2nc(N3C[C@@]4(C)CC[C@H]3CN4)c3cc(Cl)c(-c4cc(O)cc5ccccc45)c(F)c3n2)CC1] | 4-(6-chloro-2-((1- ((dimethylami- no)methyl)cyclopro- pyl)methoxy)- 8-fluoro-4-((1S,4S)- 4-methyl-2,5-diaza- bicyclo[2.2.2]octan- 2-yl)quinazolin-7- yl)naphthalen-2-ol | 576 | N/A |
| | | Peak 1 on RP-HPLC | | | |
| Ex 38b | 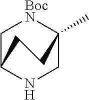 [SMILES: CC(C)(C)OC(=O)N1C[C@@H]2CC[C@]1(C)CN2] Int-LL2 | 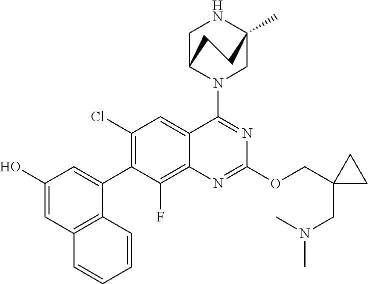 [SMILES: CN(C)CC1(COc2nc(N3C[C@@]4(C)CC[C@H]3CN4)c3cc(Cl)c(-c4cc(O)cc5ccccc45)c(F)c3n2)CC1] | 4-(6-chloro-2-((1- ((dimethyl- amino)meth- yl)cyclopropyl)meth- oxy)-8-fluoro-4- ((1S,4S)-4-methyl-2,5- diazabicyclo[2.2.2]oc- tan-2-yl)quinazolin-7- yl)naphthalen-2-ol | 576 | N/A |
| | | Peak 2 on RP-HPLC | | | |

### Example 39: 4-(6-chloro-2-((14(dimethylamino)methylcyclopropyl)methoxy)-4-((1S,4S,6R)-6-ethyl-2,5-diazabicyclo[2.2.2]octan-2-yl)-8-fluoroquinazolin-7-yl)naphthalen-2-ol (Ex. 39)

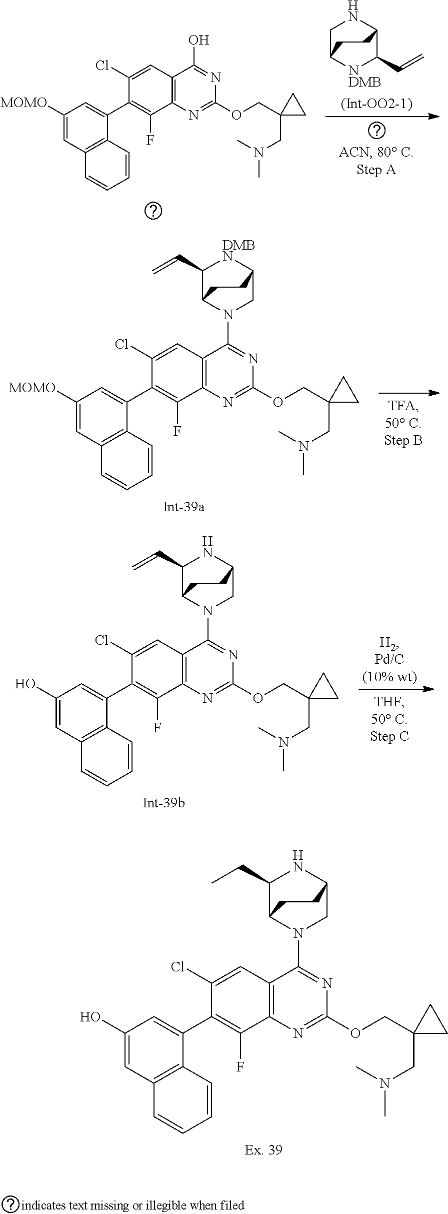

### Step A: 1-(14((6-chloro-4-((S,4S,6R)-5-(2,4-dimethoxybenzyl)-6-vinyl-2,5-diazabicyclo[2.2.2]octan-2-yl)-8-fluoro-7434methoxymethoxyl) naphthalen-1-yl)quinazolin-2-yl)oxy)methyl)cyclopropyl)-N,N-dimethylmethanamine (Int

A mixture of 6-chloro-2-((1-((dimethylamino)methyl)cyclopropyl)methoxy)-8-fluoro-7-(3-(methoxymethoxy)naphthalen-1-yl)quinazolin-4-ol (Int-V4) (75 mg, 0.12 mmol) and BOP (79 mg, 0.18 mmol) in acetonitrile (1.2 mL) was stirred at room temperature for 30 minutes. In another vial, DBU (73.0 μL, 0.48 mmol) was added to a mixture of (1S,3R,4S)-2-(2,4-dimethoxybenzyl)-3-vinyl-2,5-diazabicyclo[2.2.2]octane (Int-QQ2-1) (58 mg, 0.18 mmol) in acetonitrile (0.50 mL). The contents of this vial were then added to the first mixture and heated to 80° C. for 16 h. After 16 h, the mixture was allowed to cool to room temperature and then added to a mixture of water and DCM. The organic layer was separated and then concentrated under reduced pressure to afford 1-(1-(((6-chloro-4-((1S,4S,6R)-5-(2,4-dimethoxybenzyl)-6-vinyl-2,5-diazabicyclo[2.2.2]octan-2-yl)-8-fluoro-7-(3-(methoxymethoxy)naphthalen-1-yl)quinazolin-2-yl)oxy)methyl)cyclopropyl)-N,N-dimethylmethanamine (Int-39a), which was used in future transformations without purification. MS (ESI): m/z (M+H)⁺ 782.

### Step B: 4-(6-chloro-2-((1-((dimethylamino)methyl)cyclopropyl)methoxy)-8-fluoro-4-((1S,4S,6R)-6-vinyl-2,5-diazabicyclo[2.2.2]octan-2-yl)quinazolin-7-yl)naphthalen-2-ol (Int-39b)

TFA (0.20 mL, 2.6 mmol) was added to 1-(1-(((6-chloro-4-((1S,4S,6R)-5-(2,4-dimethoxy benzyl)-6-vinyl-2,5-diazabicyclo[2.2.2]octan-2-yl)-8-fluoro-7-(3-(methoxymethoxy)naphthalen-1-yl)quinazolin-2-yl)oxy)methyl)cyclopropyl)-N,N-dimethylmethanamine (Int-39a) (94 mg, 0.11 mmol) and 1.3-dimethoxybenzene (0.14 mL, 1.1 mmol), and the reaction mixture was heated to 80° C. for 1 hour. After 1 hour, the mixture was allowed to cool to room temperature and then concentrated under reduced pressure. The resulting mixture was purified by preparatory reverse phase HPLC (MeCN/water with 0.1% TFA modifier) to afford 4-(6-chloro-2-((1-((dimethylamino)methyl)cyclopropyl)methoxy)-8-fluoro-4-((1S,4S,6R)-6-vinyl-2,5-diazabicyclo[2.2.2]octan-2-yl)quinazolin-7-yl)naphthalen-2-ol (Int-39b). MS (ESI): m/z (M+H)⁺ 588.

### Step C: 4-(6-chloro-2-((1-((dimethylamino)methyl)cyclopropyl)methoxy)-4-((1S,4S,6R)-6-ethyl-2,5-diazabicyclo[2.2.2]octan-2-yl)-8-fluoroquinazolin-7-yl)naphthalen-2-ol (Ex. 39)

A vial containing 4-(6-chloro-2-((1-((dimethylamino)methyl)cyclopropyl)methoxy)-8-fluoro-4-((1S,4S,6R)-6-vinyl-2,5-diazabicyclo[2.2.2]octan-2-yl)quinazolin-7-yl)naphthalen-2-ol (Int-39b) (21 mg, 0.030 mmol) and THF (0.60 mL) was alternately purged with N₂ and evacuated three times. Palladium on carbon (10% loading, 6.4 mg, 6.0 μmol) was added. The N₂ line was switched to a H₂ gas balloon and the flask was alternately flushed with H₂ and vacuum twice and then H₂ was allowed to flow freely. The mixture was stirred at room temperature for 2 hours. The reaction was then filtered through a bed of CELITE and the filter bed was washed with fresh methanol. The filtrate was concentrated under reduced pressure. The crude mixture was purified by preparatory reverse phase HPLC (MeCN/water with 0.1% ammonium hydroxide modifier) to afford 4-(6-chloro-2-((1-((dimethylamino)methyl)cyclopropyl)methoxy)-4-((1S,4S,61)-6-ethyl-2,5-diazabicyclo[2.2.2]octan-2-yl)-8-fluoroquinazolin-7-yl)naphthalen-2-ol (Ex. 39) as a mixture of atropisomers. MS (ESI): m/z (M+H)⁺ 590. ¹H NMR (600 MHz, DMSO-d₆) δ 10.21 (s, 1H), 8.12 (s, 1H), 7.80 (d, J=8.3 Hz, 1H), 7.45-7.41 (m, 1H), 7.28-7.26 (m, 1H), 7.24-7.16 (m, 2H), 7.08-7.05 (m, 1H), 4.65 (s, 1H), 4.25-4.17 (m, 3H), 4.13-4.06 (m, 1H), 3.26-3.21 (m, 1H), 3.10 (s, 1H), 2.41-2.34 (m, 1H), 2.22 (s, 2H), 2.15 (s, 6H), 2.02-1.95 (m, 2H), 1.83-1.76 (m, 1H), 1.68-1.60 (m, 1H), 1.50-1.42 (m, 2H). 0.93 (t, J=7.2 Hz, 3H). 0.63-0.59 (m, 2H), 0.41-0.36 (m, 2H).

The compound in the table below was synthesized via a similar route as Ex. 39, using Int-OO2-2 as the starting material.

| | Starting | | | [M + H]⁺ |
|---|---|---|---|---|
| Ex | Material | Structure | Compound Name | Found |
| Ex 40 | 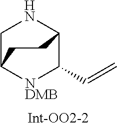 | 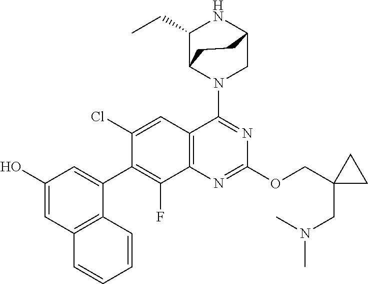 [SMILES: CC[C@@H]1N[C@H]2CC[C@@H]1N(c1nc(OCC3(CN(C)C)CC3)nc3c(F)c(-c4cc(O)cc5ccccc45)c(Cl)cc13)C2] | 4-(6-chloro-2-((1- ((dimethylamino)methyl) cyclopropyl)methoxy)-4- ((1S,4S,6S)-6-ethyl-2,5- diazabicyclo[2.2.2] octan-2-yl)-8- fluoroquinazolin-7- yl)naphthalen-2-ol | 590 |
| Ex.124 | 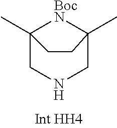 | 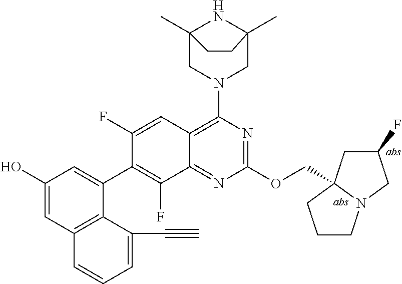 [SMILES: C#Cc1cccc2cc(O)cc(-c3c(F)cc4c(N5CC6(C)CCC(C)(C5)N6)nc(OC[C@@]56CCCN5C[C@H](F)C6)nc4c3F)c12] | 4-(4-(1,5-dimethyl-3,8- diazabicyclo[3.2.1] octan-3-yl)-6,8- difluoro-2-(((2R,7aS)-2- fluorotetrahydro- 1H-pyrrolizin-7a(5H)- yl)methoxy)quinazolin- 7-yl)-5-ethynylnaphthalen- 2-ol | 628 |

### Example 41: 3-(6-chloro-2-((1-((dimethylamino)methyl)cyclopropyl)methoxy)-8-fluoro-7-(3-hydroxynaphthalen-1-yl)quinazolin-4-yl)-5-methyl-3,8-diazabicyclo[3.2.1]octan-6-ol (Ex. 41)

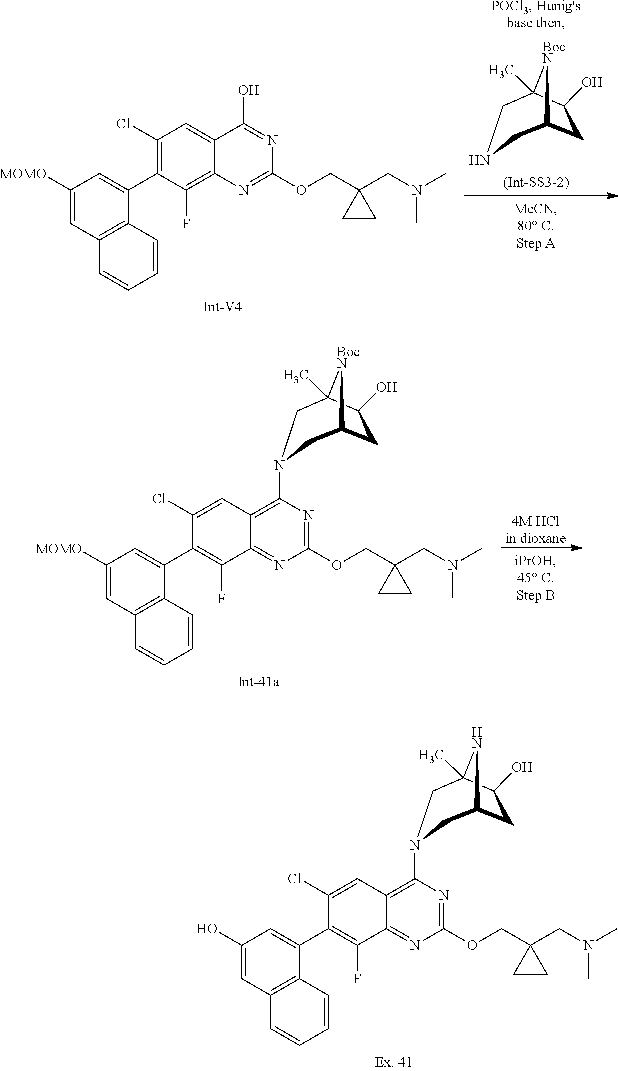

### Step A: tert-butyl-3-(6-chloro-2-((1-((dimethylamino)methyl)cyclopropyl)methoxy)-8-fluoro-7-(3-(methoxymethoxy)naphthalen-1-yl)quinazolin-4-yl)-7-hydroxy-1-methyl-3,8-diazabicyclo[3.2.1]octane-8-carboxylate (Int-41a)

A vial containing 6-chloro-2-((1-((dimethylamino)methyl)cyclopropyl)methoxy)-8-fluoro-7-(3-(methoxymethoxy)naphthalen-1-yl)quinazolin-4-ol (Int-V4) (63 mg, 0.10 mmol) was evacuated and backfilled with a balloon of nitrogen three times. Acetonitrile (0.50 mL) and DIPEA (0.18 mL, 1.0 mmol) were added to the reaction vessel. Phosphoryl trichloride (11 μL, 0.12 mmol) was added to the reaction vessel. The resulting mixture was heated to 80° C. for 30 min. After 30 minutes, the resulting mixture was cooled to room temperature and then tert-butyl 7-hydroxy-1-methyl-3,8-diazabicyclo[3.2.1]octane-8-carboxylate (Int-SS3-2) (24 mg, 0.10 mmol) was added. The resulting mixture was stirred for 1 hour at room temperature. After 1 hour, the mixture was diluted with ethyl acetate (100 mL) and then washed with saturated aqueous sodium bicarbonate (3 10 mL). The organic layer was dried over sodium sulfate, filtered and the filtrate was concentrated under reduced pressure. The residue obtained was purified by flash column chromatography with silica gel column (0-100% (3:1 ethyl acetate:ethanol) in hexanes) to afford tert-butyl-3-(6-chloro-2-((1-((dimethylamino)methyl)cyclopropyl)methoxy)-8-fluoro-7-(3-(methoxymethoxy)naphthalen-1-yl)quinazolin-4-yl)-7-hydroxy-1-methyl-3,8-diazabicyclo[3.2.1]octane-8-carboxylate (Int-41a). MS (ESI): m/z (M+H)⁺ 736.

### Step B: 3-(6-chloro-2-((1-((dimethylamino)methyl)cyclopropyl)methoxy)-8-fluoro-7-(3-hydroxynaphthalen-1-yl)quinazolin-4-yl)-5-methyl-3,8-diazabicyclo[3.2.1]octan-6-ol (Ex. 41)

A vial containing tert-butyl-3-(6-chloro-2-((1-((dimethylamino)methyl)cyclopropyl)methoxy)-8-fluoro-7-(3-(methoxymethoxy)naphthalen-1-yl)quinazolin-4-yl)-7-hydroxy-1-methyl-3,8-diazabicyclo[3.2.1]octane-8-carboxylate (Int-41a) (19 mg, 0.025 mmol) was evacuated and backfilled with a balloon of nitrogen three times. 2-Propanol (0.56 mL) was added to the vial followed by HCl (4.0 M in dioxane, 0.28 mL) dropwise. The resulting mixture was heated to 45° C. for 1 h. After 1 hour, the mixture was allowed to cool to room temperature and then concentrated under reduced pressure. The residue obtained was purified by preparative reverse phase HPLC (MeCN/water with 0.1% TFA modifier) to afford 3-(6-chloro-2-((1-((dimethylamino)methyl)cyclopropyl)methoxy)-8-fluoro-7-(3-hydroxynaphthalen-1-yl)quinazolin-4-yl)-5-methyl-3,8-diazabicyclo[3.2.1]octan-6-ol (Ex. 41). MS (ESI): m/z (M+H)⁺ 592. ¹H NMR (500 MHz, DMSO-d) S 10.08 (s, 1H). 8.01 (s, 1H). 7.83 (d, J=8.4 Hz, 1H), 7.48-7.43 (m, 1H), 7.31 (s, 1H), 7.27-7.21 (m, 1H), 7.20-7.14 (m, 1H), 7.05 (dd, J=6.6, 2,3 Hz, 1H), 5.62 (s, 1H), 4.57-4.50 (m, 1H), 4.42-4.34 (m, 2H), 4.32-4.27 (m, 2H), 4.27-4.19 (m, 2H), 3.78-3.67 (m, 2H), 3.62-3.55 (m, 2H), 2.86 (s, 3H), 2.85 (s, 3H), 1.93-1.84 (m, 2H), 1.42 (s, 3H), 0.91-0.85 (m, 2H), 0.83-0.78 (m, 2H).

Compounds in the table below were synthesized via an analogous procedure as for Ex. 41 above.

| | | | | [M + H]⁺ |
|---|---|---|---|---|
| Ex | Starting Material | Structure | Compound Name | Found |
| Ex 42 | 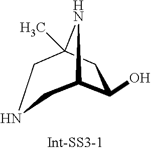 | 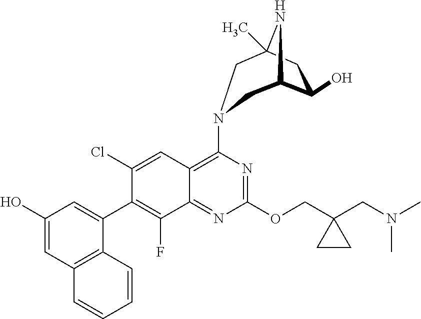 | 3-(6-chloro-2-((1- ((dimethylamino) methyl)cyclopropyl) methoxy)-8-fluoro-7- (3-hydroxynaphthalen-1- yl)quinazolin-4-yl)-1- methyl-3,8-diazabicyclo [3.2.1]octan-6-ol | 592 |
| Ex 43 | 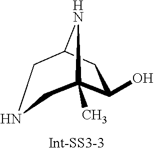 | 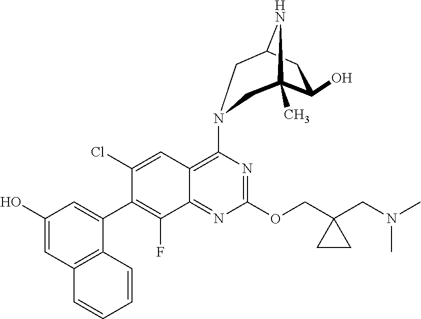 | 3-(6-chloro-2-((1- ((dimethylamino) methyl)cyclopropyl) methoxy)-8-fluoro-7- (3-hydroxynaphthalen-1- yl)quinazolin-4-yl)-5- methyl-3,8-diazabicyclo [3.2.1]octan-6-ol | 592 |
| Ex 44 | 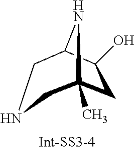 | 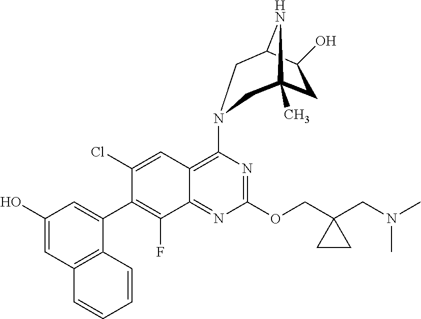 | (1S,5R,6S)-3- (6-chloro-2-((1- ((dimethylamino) methyl)cyclopropyl) methoxy)-8-fluoro-7- (3-hydroxynaphthalen-1- yl)quinazolin-4-yl)-1- methyl-3,8-diazabicyclo [3.2.1]octan-6-ol | 592 |

### Example 45: 3-(4-(3,8-diazabicyclo[3.2.1]octan-3-yl)-6-chloro-2-((1-((dimethylamino)methyl)cyclopropyl)methoxy)-8-fluoroquinazolin-7-yl)-4-(trimethylsilyl)phenol (Ex. 45)

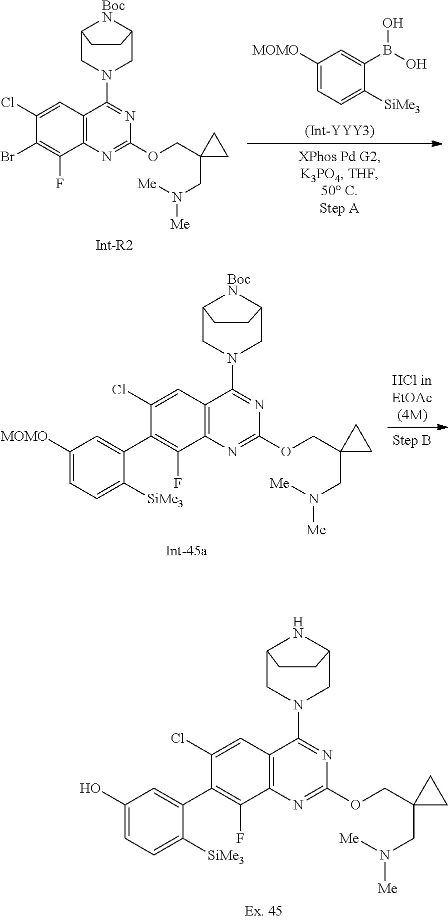

### Step A; tert-butyl 346-chloro-2-((1-((dimethylamino)methyl)cyclopropyl)methoxy)-8-fluoro-7-(5-(methoxymethoxy)-2-(trimethylsilyl)phenyl)quinazolin-4-yl)-3,8-diazabicyclo[3.2.1]octane-8-carboxylate (Int-45a)

To a mixture of tert-butyl 3-(7-bromo-6-chloro-2-((1-((dimethylamino)methyl)cyclopropyl)methoxy)-8-fluoroquinazolin-4-yl)-3,8-diazabicyclo[3.2.1]octane-8-carboxylate (Int-R2) (35 mg, 0.058 mmol) and (5-(methoxymethoxy)-2-(trimethylsilyl)phenyl)boronic acid (Int-YYY2) (44.6 mg. 0.175 mmol) in THF (1 mL) were added aqueous potassium phosphate (0.117 mL, 0.175 mmol) (1.5 M), followed by XPhos Pd G2 (9.2 mg, 0.012 mmol). The mixture was stirred at 50° C. for 16 h. The reaction mixture was purified by preparative TLC plate directly eluting with 10% MeOH in DCM to give tert-butyl 3-(6-chloro-2-((1-((dimethylamino)methyl)cyclopropyl)methoxy)-8-fluoro-7-(5-(methoxymethoxy)-2-(trimethylsilyl)phenyl)quinazolin-4-yl)-3,8-diazabicyclo[3.2.1]octane-8-carboxylate (Int-45a). MS (ESI): m/z (M+H)⁺ 728.

### Step B: 3(4-(3.8-diazabicyclo[3.2.1]octan-3-yl)-6-chloro-2-((I-((dimethylamino)methyl)cyclopropyl)methyl)-8-fluoroquinazolin-7-yl)-4-(trimethylsilyl)phenol (Ex. 45)

A solution of tert-butyl 3-(6-chloro-2-((1-((dimethylamino)methyl)cyclopropyl)methoxy)-8-fluoro-7-(5-(methoxymethoxy)-2-(trimethylsilyl)phenyl)quinazolin-4-yl)-3,8-diazabicyclo[3.2.1]octane-8-carboxylate (Int-45a) (10 mg, 0.014 mmol) in HCl/EtOAc (1 mL, 4.00 mmol) (4 M in EtOAc) was stirred at 20° C. for 30 min. The reaction mixture was concentrated in vacuo, and the residue was purified by mass triggered reverse phase HPLC (MeCN/water with 0.05% NH₄HCO₃ modifier) to afford 3-(4-(3.8-diazabicyclo[3.2.1.]octan-3-yl)-6-chloro-2-((1-((dimethylamino)methyl)cyclopropyl)methoxy)-8-fluoroquinazolin-7-yl)-4-(trimethylsilyl)phenol (Ex. 45). MS (ESI): m/z (M+H)⁺ 584. ¹H NMR (500 MHz, MeOD) δ 7.83 (s, 1H), 7.50 (d, J=8.2 Hz, 1H), 6.88 (dd, J=2,4, 8.2 Hz, 1H), 6.57 (d, J=2.4 Hz, 1H), 4.43 (br dd, J=4.1, 10.5 Hz, 2H), 4.32 (s, 2H), 3.65-3.56 (m, 4H), 2.54-2.43 (m, 2H), 2.32 (s, 6H), 1.90-1.79 (m, 4H), 0.76-0.69 (m, 2H), 0.56-0.49 (m, 2H), -0.06 (s, 9H).

Compounds in the table below were made via a similar synthetic sequence as Ex. 45 above.

| | | | | [M + H]⁺ |
|---|---|---|---|---|
| Ex. | Starting Material | Structure | Compound Name | Found |
| Ex 46 | 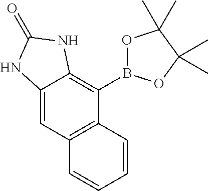 [SMILES: CC1(C)OB(c2c3ccccc3cc3[nH]c(=O)[nH]c23)OC1(C)C] | 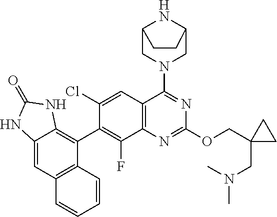 [SMILES: CN(C)CC1(COc2nc(N3CC4CCC(C3)N4)c3cc(Cl)c(-c4c5ccccc5cc5[nH]c(=O)[nH]c45)c(F)c3n2)CC1] | 4-(4-(3,8-diazabicyclo[3.2.1]octan-3- yl)-6-chloro-2-((1- ((dimethylamino)methyl)cyclopropyl) methoxy)-8-fluoroquinazolin-7-yl)-1,3- dihydro-2H-naphtho[2,3-d]imidazol-2- one | 602 |
| | Int-WWW4 | | | |
| Ex 47 | 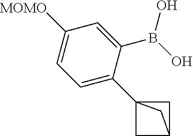 | 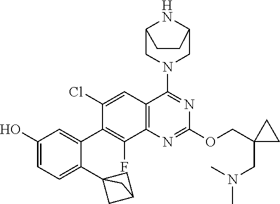 [SMILES: CN(C)CC1(COc2nc(N3CC4CCC(C3)N4)c3cc(Cl)c(-c4cc(O)ccc4C45CC(C4)C5)c(F)c3n2)CC1] | 3-(4-(3,8-diazabicyclo[3.2.1]octan-3- yl)-6-chloro-2-((1- ((dimethylamino)methyl)cyclopropyl) methoxy)-8-fluoroquinazolin-7-yl)-4- (bicyclo[1.1.1]pentan-1-yl)phenol | 578 |
| | Int-XXX3 | | | |
| Ex 48 | 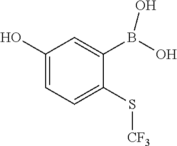 [SMILES: OB(O)c1cc(O)ccc1SC(F)(F)F] | 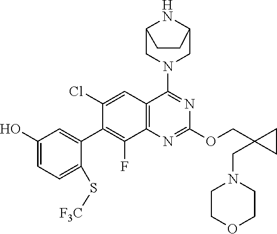 [SMILES: Oc1ccc(SC(F)(F)F)c(-c2c(Cl)cc3c(N4CC5CCC(C4)N5)nc(OCC4(CN5CCOCC5)CC4)nc3c2F)c1] | 3-(4-(3,8-diazabicyclo[3.2.1]octan-3- yl)-6,8-difluoro-2-((1- (morpholinomethyl)cyclopropyl)methoxy) quinazolin-7-yl)-4- ((trifluoromethyl)thio)phenol | 638 |
| | Int-OOO5 | | | |
| Ex 49 | 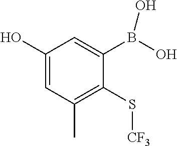 [SMILES: Cc1cc(O)cc(B(O)O)c1SC(F)(F)F] | 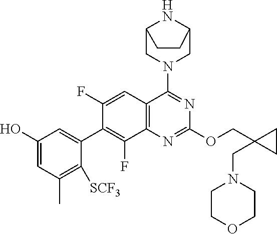 | 3-(4-(3,8-diazabicyclo[3.2.1]octan-3- yl)-6,8-difluoro-2-((1- (morpholinomethyl)cyclopropyl)methoxy) quinazolin-7-yl)-5-methyl-4- ((trifluoromethyl)thio)phenol | 652 |
| | Int-PPP2 | | | |
| Ex 50 | 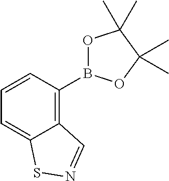 [SMILES: CC1(C)OB(c2cccc3sncc23)OC1(C)C] | 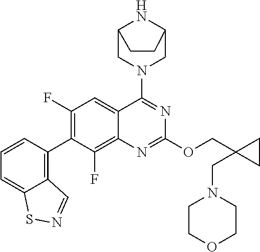 [SMILES: Fc1cc2c(N3CC4CCC(C3)N4)nc(OCC3(CN4CCOCC4)CC3)nc2c(F)c1-c1cccc2sncc12] | 4-((1-(((7-(benzo[d]isothiazol-4-yl)-4- (3,8-diazabicyclo[3.2.1]octan-3-yl)-6,8- difluoroquinazolin-2- yl)oxy)methyl)cyclopropyl)methyl) morpholine | 579 |
| | Int-QQQ2 | | | |
| Ex 51 | 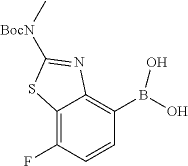 [SMILES: CN(C(=O)OC(C)(C)C)c1nc2c(B(O)O)ccc(F)c2s1] | 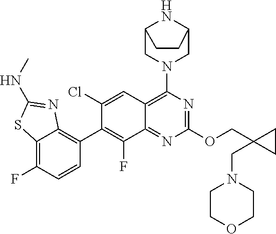 [SMILES: CNc1nc2c(-c3c(Cl)cc4c(N5CC6CCC(C5)N6)nc(OCC5(CN6CCOCC6)CC5)nc4c3F)ccc(F)c2s1] | 4-(4-(3,8-diazabicyclo[3.2.1]octan-3- yl)-6-chloro-8-fluoro-2-((1- (morpholinomethyl)cyclopropyl)methoxy) quinazolin-7-yl)-7-fluoro-N- methylbenzo[d]thiazol-2-amine | 642 |
| | Int-RRR4 | | | |
| Ex 52 | 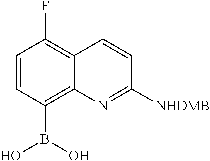 | 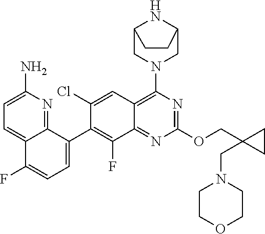 [SMILES: Nc1ccc2c(F)ccc(-c3c(Cl)cc4c(N5CC6CCC(C5)N6)nc(OCC5(CN6CCOCC6)CC5)nc4c3F)c2n1] | 8-(4-(3,8-diazabicyclo[3.2.1]octan-3- yl)-6-chloro-8-fluoro-2-((1- (morpholinomethyl)cyclopropyl)methoxy) quinazolin-7-yl)-5-fluoroquinolin-2- amine | 622 |
| | Int-SSS5 | | | |
| Ex 53 | 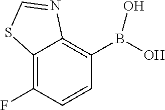 [SMILES: OB(O)c1ccc(F)c2scnc12] | 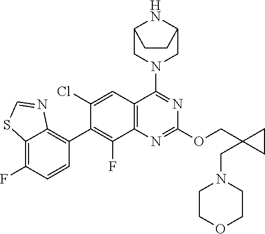 [SMILES: Fc1c(-c2ccc(F)c3scnc23)c(Cl)cc2c(N3CC4CCC(C3)N4)nc(OCC3(CN4CCOCC4)CC3)nc12] | 4-((1-(((4-(3,8- diazabicyclo[3.2.1]octan-3-yl)-6-chloro- 8-fluoro-7-(7-fluorobenzo[d]thiazol-4- yl)quinazolin-2- yl)oxy)methyl)cyclopropyl)methyl) morpholine | 613 |
| | Int-UUU4 | | | |
| Ex 54 | 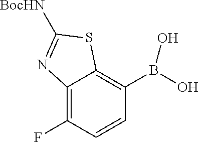 [SMILES: CC(C)(C)OC(=O)Nc1nc2c(F)ccc(B(O)O)c2s1] | 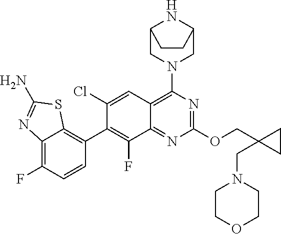 [SMILES: Nc1nc2c(F)ccc(-c3c(Cl)cc4c(N5CC6CCC(C5)N6)nc(OCC5(CN6CCOCC6)CC5)nc4c3F)c2s1] | 7-(4-(3,8-diazabicyclo[3.2.1]octan-3- yl)-6-chloro-8-fluoro-2-((1- (morpholinomethyl)cyclopropyl)methoxy) quinazolin-7-yl)-4- fluorobenzo[d]thiazol-2-amine | 628 |
| | Int-VVV2 | | | |
| Ex 55 | 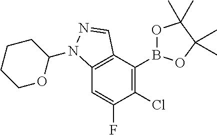 [SMILES: CC1(C)OB(c2c(Cl)c(F)cc3c2cnn3C2CCCCO2)OC1(C)C] | 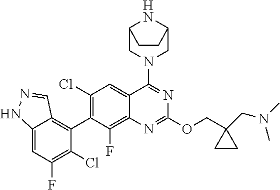 [SMILES: CN(C)CC1(COc2nc(N3CC4CCC(C3)N4)c3cc(Cl)c(-c4c(Cl)c(F)cc5[nH]ncc45)c(F)c3n2)CC1] | 1-(1-(((4-(3,8-diazabicyclo[3.2.1]octan- 3-yl)-6-chloro-7-(5-chloro-6-fluoro-1H- indazol-4-yl)-8-fluoroquinazolin-2- yl)oxy)methyl)cyclopropyl)-N,N- dimethylmethanamine | 588 |
| | Int-AD2 | | | |
| Ex 56 | 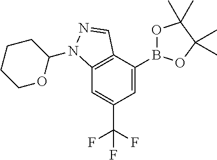 [SMILES: CC1(C)OB(c2cc(C(F)(F)F)cc3c2cnn3C2CCCCO2)OC1(C)C] | 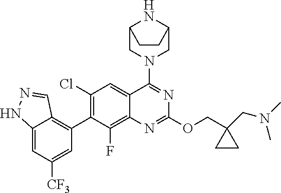 [SMILES: CN(C)CC1(COc2nc(N3CC4CCC(C3)N4)c3cc(Cl)c(-c4cc(C(F)(F)F)cc5[nH]ncc45)c(F)c3n2)CC1] | 1-(1-(((4-(3,8-diazabicyclo[3.2.1]octan- 3-yl)-6-chloro-8-fluoro-7-(6- (trifluoromethyl)-1H-indazol-4- yl)quinazolin-2- yl)oxy)methyl)cyclopropyl)-N,N- dimethylmethanamine | 604 |
| | Int-AC2 | | | |
| Ex 57 | 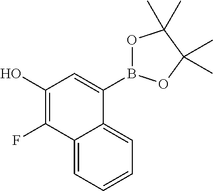 [SMILES: CC1(C)OB(c2cc(O)c(F)c3ccccc23)OC1(C)C] | 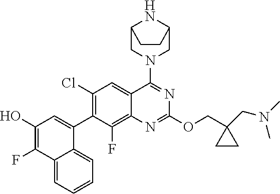 [SMILES: CN(C)CC1(COc2nc(N3CC4CCC(C3)N4)c3cc(Cl)c(-c4cc(O)c(F)c5ccccc45)c(F)c3n2)CC1] | 4-(4-(3,8-diazabicyclo[3.2.1]octan-3- yl)-6-chloro-2-((1- ((dimethylamino)methyl)cyclopropyl) methoxy)-8-fluoroquinazolin-7-yl)-1- fluoronaphthalen-2-ol | 580 |
| | Int-AB1 | | | |
| Ex 58 | 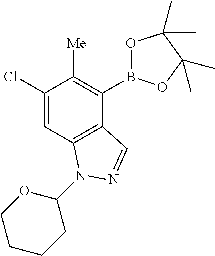 [SMILES: Cc1c(Cl)cc2c(cnn2C2CCCCO2)c1B1OC(C)(C)C(C)(C)O1] | 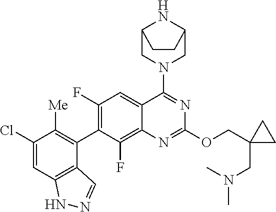 [SMILES: Cc1c(Cl)cc2[nH]ncc2c1-c1c(F)cc2c(N3CC4CCC(C3)N4)nc(OCC3(CN(C)C)CC3)nc2c1F] | 1-(1-(((4-(3,8-diazabicyclo[3.2.1]octan- 3-yl)-7-(6-chloro-5-methyl-1H-indazol- 4-yl)-6,8-difluoroquinazolin-2- yl)oxy)methyl)cyclopropyl)-N,N- dimethylmethanamine | 568 |
| | Int-AE1 | | | |
| Ex 59 | 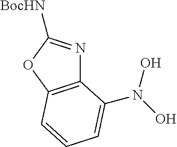 [SMILES: CC(C)(C)OC(=O)Nc1nc2c(N(O)O)cccc2o1] | 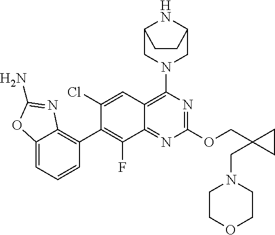 [SMILES: Nc1nc2c(-c3c(Cl)cc4c(N5CC6CCC(C5)N6)nc(OCC5(CN6CCOCC6)CC5)nc4c3F)cccc2o1] | 4-(4-(3,8-diazabicyclo[3.2.1]octan-3- yl)-6-chloro-8-fluoro-2-((1- (morpholinomethyl)cyclopropyl)methoxy) quinazolin-7-yl)benzo[d]oxazol-2- amine | 594 |
| | Int-TTT3 | | | |

### Example 60:: 8(4-((R,5S)3,8-diazabicyclo[3.2.1]octan-3-yl)-6-chloro-2-((1-((dimethylamino)methyl)cyclopropyl)methoxy)-8-fluoroquinazolin-7-yl)-1-naphthonitrile (Ex. 60)

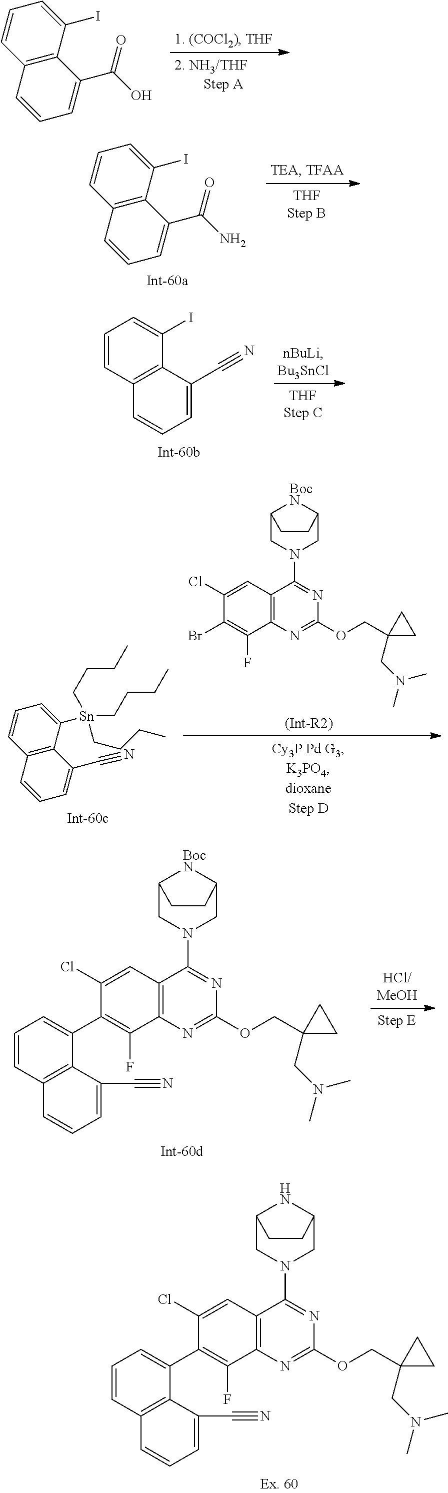

### Step A: 8-iodo-_-naphthamide (Int-60a)

To a stirred solution of 8-iodo-1-naphthoic acid (2.0 g, 6.71 mmol) in THF (10 mL) were added DMF (0.052 mL, 0.671 mmol) and oxalyl dichloride (2.55 g, 20.13 mmol) at 0° C., and the reaction mixture was stirred at 25° C. for 2 h. The reaction mixture was concentrated in vacuo. The residue was dissolved in DCM (20 mL), and the solution was added into a solution of NH₃/THF (4M, 10 mL) at 0° C., and the resulting mixture was stirred at 25° C. for 1 h. The reaction mixture was concentrated in vacuo, and the residue was purified by flash silica gel chromatography (Eluent of 0˜60%/EtOAc/Pet, ether) to give 8-iodo-1-naphthamide (Int-60a). ¹H NMR (400 MHz, DMSO-d₆) δ 8.24 (dd, J=1.2, 7.4 Hz, 1H), 8.05 (br s, 1H), 8.03-7.93 (m, 2H), 7.58-7.46 (m, 3H), 7.22 (t, J=7.8 Hz, 1H).

### Step B: 8-iodo-1-naphthonitrile (Int-60b)

To a stirred solution of 8-iodo-1-naphthamide (Int-60a) (1.85 g, 6.23 mmol) and TEA (3.47 mL, 24.9 mmol) in THF (15 mL) was added TFA A (3.52 mL. 24.9 mmol) at 0° C. and the mixture was stirred at 20° C. for 2 h. The mixture was concentrated in vacuo, and the residue was purified by flash silica gel chromatography (Eluent of 0-20% EtOAc/Pet, ether) to give 8-iodo-1-naphthonitrile (Int-60b). ¹H NMR (400 MHz, CDCl₃) δ 8.34 (d, J=7.4 Hz. 1H). 8.08 (dd, J=7.6, 13.5 Hz. 2H), 7.91 (d, J=7.8 Hz. 1H), 7.54 (t, J=7.6 Hz, 1H), 7.23 (t, J=7.8 Hz, 1H).

### Step C: 8-(tributylstannyl)-1-naphthonitrile (Int-60c)

To a stirred solution of 8-iodo-1-naphthonitrile (Int-60b) (500 mg. 1.792 mmol) in THF (5 mL) was added n-butylthium (0.860 mL, 2.150 mmol) (2.5 M in Hexane) at −78° C. The reaction mixture was stirred at −78° C. for 30 min. Then tributyltin chloride (0.729 mL, 2.69 mmol) was added, and the reaction mixture was warmed to 25° C. and stirred at this temperature for 1 h. The reaction mixture was quenched with saturated aqueous NH₄Cl (5 mL), and extracted with EtOAc (50 mL). The aqueous layer was extracted with EtOAc (50 mL×3). The combined organic layers were washed with brine (5 mL), dried over Na₂SO₄ and filtered. The filtrate was concentrated under reduced pressure to give the residue, which was purified by Column chromatography on silica gel (Pet, ether/EtOAc=20/1) to yield 8-(tributylstannyl)-1-naphthonitrile (Int-60c).

¹H NMR (400 MHz. MeOD) δ 8.21-8.12 (m, 1H), 8.07-7.90 (m, 2H), 7.87-7.48 (m, 3H), 1.74-1.45 (m, 6H), 1.44-1.14 (m, 12H), 0.99-0.76 (m, 9H).

### Step D: tert-butyl (1R,5S)-3-(6-chloro-7-(8-cyanonaphthalen-1-yl)-2-((1-((dimethylamino)methyl)cyclopropyl)methoxy)-8-fluoroquinazolin-4-yl)-3,8-diazabicyclo[3.2.1]octane-8-carboxylate (Int-60d)

To a mixture of tert-butyl 3-(7-bromo-6-chloro-2-((1-((dimethylamino)methyl)cyclopropyl)methoxy)-8-fluoroquinazolin-4-yl)-3,8-diazabicyclo[3.2.1]octane-8-carboxylate (Int-R2) (100 mg, 0.167 mmol) and 8-(tributylstannyl)-1-naphthonitrile (Int-60c) (148 mg, 0.334 mmol) in dioxane (3 mL) were added potassium phosphate (70.9 mg, 0.334 mmol) and methanesulfonato(tricyclohexylphosphine)(2′-amino-1,1′-biphenyl-2-yl)palladium(II) DCM adduct (49.1 mg. 0.067 mmol) at 25° C. in a glovebox, and the mixture was stirred at 80° C. for 48 h. The reaction mixture was concentrated in vacuo and the residue was purified by preparative TLC plate (basified with Et₃N. Pet, ether/EtOAc=2/1) to give tert-butyl (1R,5S)-3-(6-chloro-7-(8-cyanonaphthalen-1-yl)-2-((1-((dimethylamino)methyl)cyclopropyl)methoxy)-8-fluoroquinazolin-4-yl)-3,8-diazabicyclo[3.2.1]octane-8-carboxylate (Int-60d). MS (ESI): m/z (M+H)⁺ 671.

### Step E: 8-(4-((1R,5S)-3,8-diazabicyclo[3.2.1]octan-3-yl)-6-chloro-2-((1-((dimethylamino)methyl)cyclopropyl)methoxy)-8-fluoroquinazolin-7-yl)-1-naphthonitrile (Ex. 60)

To a mixture of tert-butyl (1R, 5S)-3-(6-chloro-7-(8-cyanonaphthalen-1-yl)-2-((1-((dimethylamino)methyl)cyclopropyl)methoxy)-8-fluoroquinazolin-4-yl)-3,8-diazabicyclo[3.2.1]octane-8-carboxylate (Int-60d) (30 mg, 0.040 mmol) in MeOH (3 mL) was added HCl/MeOH (1 mL, 4M). The mixture was stirred at 25° C. for 0.5 h. The reaction mixture was filtered and the filtrate was purified by preparative HPLC (MeCN/water with 0.2% formic acid modifier) to give 8-(4-((1R,5S)-3,8-diazabicyclo[3.2.1]octan-3-yl)-6-chloro-2-((1-((dimethylamino)methyl)cyclopropyl)methyl)-8-fluoroquinazolin-7-yl)-1-naphthonitrile (Ex. 60). MS (ESI): m/z (M+H)⁺ 571. ¹H NMR (500 MHz, MeOD) δ 8.42 (d, J=7.5 Hz, 1H), 8.26 (d, J=7.6 Hz, 1H), 8.06 (dd, J=1.1, 7.2 Hz, 1H), 8.00 (s, 1H), 7.88-7.81 (m, 1H) 7.78-7.69 (m, 1H), 7.68-7.61 (m, 1H), 4.76-4.57 (m, 3H), 4.54-4.47 (m, 1H), 4.43-4.34 (m, 1H), 4.08 (br s, 2H), 3.85 (br t, J=14.3 Hz, 2H), 3.27-3.19 (m, 1H), 3.01-2.89 (m, 6H), 2.16-1.92 (m, 4), J=0.95 (m, 2H). 0.93-0.83 (m, 2H).

### Example 61: 4-(4-(3.8-diazabicyclo[3,2,1]octan-3-yl)-6,8-difluoro-2-((1-(((J=3-fluoropyrrolidin-1-yl)methyl)cyclopropyl)methoxy)quinazolin-7-yl)-7-fluorobenzo[d]thiazol-2-amine (Ex. 61)

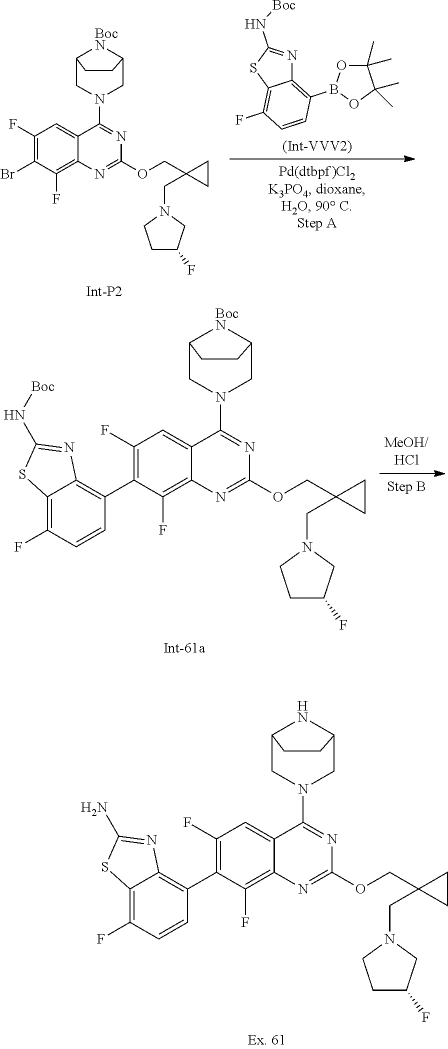 [SMILES: CC(C)(C)OC(=O)N1C2CCC1CN(c1nc(OCC3(CN4CC[C@@H](F)C4)CC3)nc3c(F)c(Br)c(F)cc13)C2 ; CC(C)(C)OC(=O)Nc1nc2c(B3OC(C)(C)C(C)(C)O3)ccc(F)c2s1 ; CC(C)(C)OC(=O)Nc1nc2c(-c3c(F)cc4c(N5CC6CCC(C5)N6C(=O)OC(C)(C)C)nc(OCC5(CN6CC[C@@H](F)C6)CC5)nc4c3F)ccc(F)c2s1 ; Nc1nc2c(-c3c(F)cc4c(N5CC6CCC(C5)N6)nc(OCC5(CN6CC[C@@H](F)C6)CC5)nc4c3F)ccc(F)c2s1]

### Step A: tert-butyl 3-(7-(2-((tert-butoxycarbonyl)amino)-7-fluorobenzo[d]thiazol-4-yl)-6,8-difluoro-2-((1-((R)-3-fluoropyrrolidin-1-yl)methyl)cyclopropyl)methoxy) quinazolin-4-yl)-3,8-diazabicyclo[3.2.1]octane-8-carboxylate (Int-61a)

To a solution of tert-butyl 3-(7-bromo-6,8-difluoro-2-((1-(((R)-3-fluoropyrrolidin-1-yl)methyl)cyclopropyl)methoxy)quinazolin-4-yl)-3,8-diazabicyclo[3.2.1]octane-8-carboxylate (Int-P2) (38 mg, 0.061 mmol) in dioxane (1.5 mL) and water (0.15 mL) were added tert-butyl (7-fluoro-4-(4,4,5,5-tetramethyl-1,3,2-dioxaborolan-2-yl)benzo[d]thiazol-2-yl)carbamate (Int-VVV2) (38.3 mg, 0.097 mmol), potassium phosphate (38.6 mg, 0.182 mmol) and [1.1′-bis(di-tert-butylphosphino)ferrocene]dichloropalladium(II) (7.91 mg, 0.012 mmol) at 25° C. under a N₂ atmosphere. The mixture was stirred at 90° C. for 2 h. The mixture was cooled and the solvent was evaporated under reduced pressure to give the crude product. The residue was purified by reverse phase preparative HPLC (MeCN/water with 0.1% TFA modifier) to give tert-butyl 3-(7-(2-((tert-butoxycarbonyl)amino)-7-fluorobenzo[d]thiazol-4-yl)-6,8-difluoro-2-((1-(((R)-3-fluoropyrrolidin-1-yl)methyl)cyclopropyl)methoxy)quinazolin-4-yl)-3,8-diazabicyclo[3.2.1]octane-8-carboxylate (Int-61a). MS (ESI): m/z (M+H)⁺ 814.

### Step B: 4-44-(3.8-diazabicyclo[3.2.1]octan-3-yl)-6,8-difluoro-2-((1-(((R)-3-fluoropyrrolidin-1-yl)methyl)cyclopropyl)methoxy)quinazolin-7-yl)-7-fluorobenzo[d]thiazol-2-amine (Ex. 61)

A solution of tert-butyl 3-(7-(2-((tert-butoxycarbonyl)amino)-7-fluorobenzo[d]thiazol-4-yl)-6,8-difluoro-2-((1-(((R)-3-fluoropyrrolidin-1-yl)methyl)cyclopropyl)methoxy)quinazolin-4-yl)-3,8-diazabicyclo[3.2.1]octane-8-carboxylate (Int-61a) (35 mg, 0.043 mmol) in HCl/MeOH (4M) (2 mL) was stirred at 25° C. for 2 h. The mixture was evaporated under reduced pressure to give the crude product. The residue was purified by reverse phase preparative HPLC (MeCN/water with 0.2% formic acid modifier) to give 4-(4-(3.8-diazabicyclo[3.2.1]octan-3-yl)-6,8-difluoro-2-((1-(((?)-3-fluoropyrrolidin-1-yl)methyl)cyclopropyl)methoxy)quinazolin-7-yl)-7-fluorobenzo[d]thiazol-2-amine (Ex. 61). MS (ESI): m/z (M+H)⁺ 614. ¹H NMR (500 MHz, MeOD) δ 7.60 (br d, J=9.5 Hz, 1H), 7.32 (dd, J=5.5, 8.5 Hz, 1H), 7.02 (t, J=8.5 Hz, 1H), 5.45-5.25 (m, 1H), 4.68-4.30 (m, 414), 4.15 (br s, 2H), 3.89-3.74 (m, 2H), 3.58 (br d, J=15.0 Hz, 11H), 3.50-3.34 (m, 2H), 3.30-3.16 (m, 2H), 3.06-2.93 (m, 1H), 2.44-2.06 (m, 6H) 0.92-0.70 (m, 4H).

| | | | [M + H]⁺ |
|---|---|---|---|
| Ex | Structure | Compound Name | Found |
| Ex 62 | 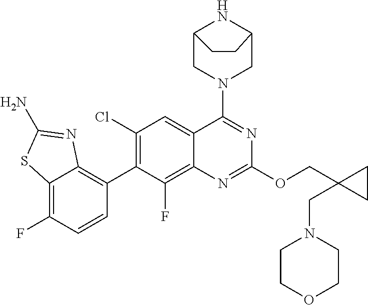 [SMILES: Nc1nc2c(-c3c(Cl)cc4c(N5CC6CCC(C5)N6)nc(OCC5(CN6CCOCC6)CC5)nc4c3F)ccc(F)c2s1] | 4-(4-(3,8-diazabicyclo[3.2.1]octan- 3-yl)-6-chloro-8-fluoro-2-((1- (morpholinomethyl)cyclopropyl) methoxy)quinazolin-7-yl)-7- fluorobenzo[d]thiazol-2-amine | 628 |
| | (boronic acid/ester: Int-VVV2) | | |
| Ex 63 | 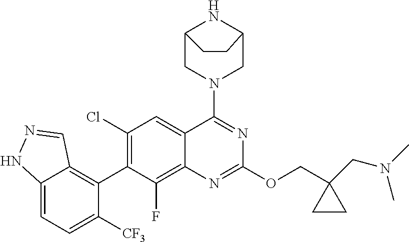 [SMILES: CN(C)CC1(COc2nc(N3CC4CCC(C3)N4)c3cc(Cl)c(-c4c(C(F)(F)F)ccc5[nH]ncc45)c(F)c3n2)CC1] | 1-(1-(((4-((1R,5S)-3,8-diazabicyclo [3.2.1]octan-3-yl)-6-chloro-8- fluoro-7-(5-(trifluoromethyl)-1H- indazol-4-yl)quinazolin-2- yl)oxy)methyl)cyclopropyl)- N,N-dimethylmethanamine | 604 |
| | (boronic acid/ester: Int-ADD2) | | |

### Example 64: 1-(1-(((4-((1R,5S)-3,8-diazabicyclo[3.2.1]octan-3-yl)-7-(5-chloro-6-methyl-1H-indazol-4-yl)-6,8-difluoroquinazolin-2-yl)oxy)methyl)-2,2-difluorocyclopropyl)-N,N-dimethylmethanamine (Ex. 64)

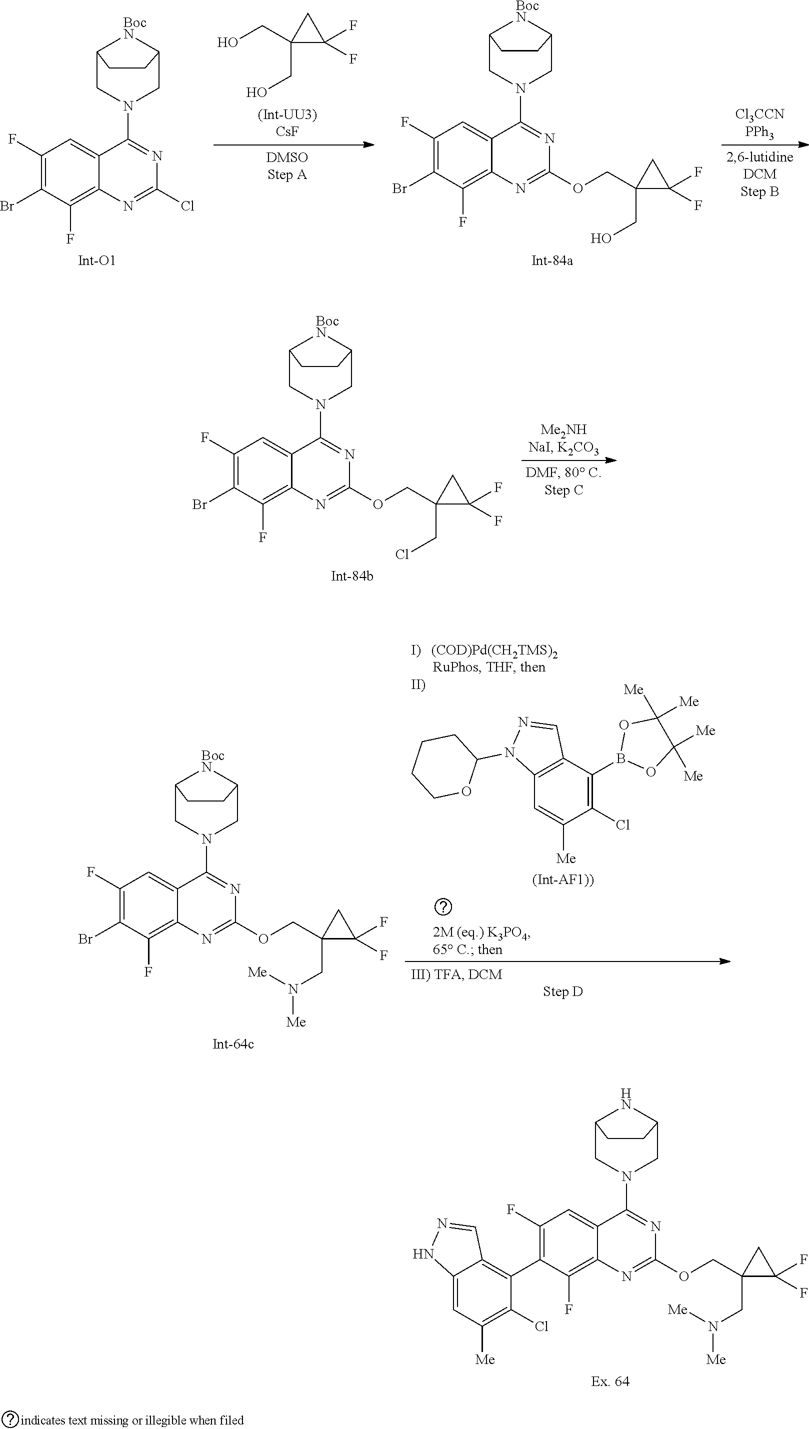

### Step A: tert-butyl 3-47-bromo-2-((2,2-difluoro-1-(hydroxymethyl)cyclopropyl)methoxy)-6,8-difluinazolin-4y 1)-3,8-diazabicyclo[3.2.1]octane-8-carbox %,late (fat-64a)

CsF (431 mg, 2.84 mmol) was added to a suspension of tert-butyl 3-(7-bromo-2-chloro-6,8-difluoroquinazolin-4-yl)-3,8-diazabicyclo[3.2.1]octane-8-carboxylate (Int-O1) (278 mg, 0.568 mmol) and [2,2-difluoro-1-(hydroxymethyl)cyclopropyl]methanol (Int-UU3) (98 mg, 0.71 mmol) in DMSO (11.4 mL). The reaction was heated to 100° C. for 20 min in a microwave reactor. The reaction was cooled to room temperature, diluted with brine (25 mL), and extracted with DCM (3×25 mL). The combined organic layers were dried over anhydrous MgSO₄, filtered, and concentrated under reduced pressure to provide tert-butyl 3-(7-bromo-2-((2,2-difluoro-1-(hydroxymethyl)cyclopropyl)methoxy)-6,8-difluoroquinazolin-4-yl)-3,8-diazabicyclo[3.2.1]octane-8-carboxylate (Int-64a), which was used in the subsequent step without further purification. MS (ESI): m: (M+H)⁺ 591/593.

### Step B: tert-butyl (1R,5S)-3-(7-bromo-2-((1-(chloromethyl)-2,2-difluorocyclopropyl)methoxy)-6,8-difluoroquinazolin-4-yl)-3,8-diazabicyclo[3.2.1]octane-8-carboxylate (Int-64b)

2,6-lutidine (83 μL, 0.71 mmol) and Cl₃CCN (71 μL, 0.71 mmol) were sequentially added to a solution of tert-butyl 3-(7-bromo-2-((2,2-difluoro-1-(hydroxymethyl)cyclopropyl)methoxy)-6,8-difluoroquinazolin-4-yl)-3,8-diazabicyclo[3.2.1]octane-8-carboxylate (Int-64a) (336 mg, 0.177 mmol) and PPh₃ (186 mg, 0.71 mmol) in DCM (5.68 mL). The reaction was stirred vigorously at room temperature for 1 h. The reaction was concentrated under reduced pressure, and the crude residue was purified by silica gel chromatography (gradient elution: 0-75% EtOAc/hexanes) to provide tert-butyl (1R,5S)-3-(7-bromo-2-((I-(chloromethyl)-2,2-difluorocyclopropyl)methoxy)-6,8-difluoroquinazolin-4-yl)-3,8-diazabicyclo[3.2.1]octane-8-carboxylate (Int-64b). MS (ESI): m/z (M+H)⁺ 609/611.

### Step C: tert-butyl (1R,5S)-3-(7-bromo-2-((1-((dimethylamino)methyl)-2,2-difluorocyclopropyl)methoxy)-6,8-difluoroquinazolin-4-yl)-3,8-diazabicyclo[3.2.1]octane-8-carboxylate (Int-64c)

Dimethylamine (0.19 mL, 0.38 mmol) was added to a suspension of tert-butyl (5. 3-(7-bromo-2-((1-(chloromethyl)-2,2-difluorocyclopropyl)methoxy)-6,8-difluoroquinazolin-4-yl)-3,8-diazabicyclo[3.2.1]octane-8-carboxylate (Int-64b) (208 mg, 0.341 mmol), K₂CO₃ (141 mg, 1.02 mmol), and Na1 (61,3 mg, 0.409 mmol) in DMF (3.41 mL). The reaction was vigorously stirred at 80° C. for 48 h. The reaction was cooled to room temperature, filtered, and concentrated under reduced pressure. The crude residue was purified by silica gel chromatography (gradient elution: 0-75% [3:1 EtOAc/EtOH]/Hexanes with 1% Et₃N) to provide tert-butyl (1R,5S)-3-(7-bromo-2-((1-((dimethylamino)methyl)-2,2-difluorocyclopropyl)methoxy)-6,8-difluoroquinazolin-4-yl)-3,8-diazabicyclo[3.2.1]octane-8-carboxylate (Int-64c). MS (ESI): m/z (M+H)⁺ 618/620.

### Step D: 1-(1-(((4-((1R,5S)-3,8-diazabicyclo[3.2.1]octan-3-yl)-7-(5-chloro-6-methyl-1H-indazol-4-yl)-6,8-difluoroquinazolin-2-yl)oxy)methyl)-2,2-difluorocyclopropyl)-N,N-dimethylmethanamine (Ex. 64)

tert-butyl (1R,5S)-3-(7-bromo-2-((1-((dimethylamino)methyl)-2,2-difluorocyclopropyl)methoxy)-6,8-difluoroquinazolin-4-yl)-3,8-diazabicyclo[3.2.1]octane-8-carboxylate (Int-64c) (30 mg. 0.05 mmol), RuPhos (23 mg, 0.05 mmol), and (COD)Pd(CH₂TMS)₂ (19 mg, 0.05 mmol) were combined. The reaction vessel was sealed and flushed with nitrogen for 5 min, evacuated for 1 min, and backfilled with nitrogen for 1 min. Degassed THF (0.5 mL) was added, and the reaction was stirred vigorously at room temperature for 16 h. 4-bromoanisole(12 μL, 0.10 mmol), 5-chloro-6-methyl-1-(tetrahydro-2H-pyran-2-yl)-4-(4,4,5,5-tetramethyl-1,3,2-dioxaborolan-2-yl)-1H-indazole (Int-AF1) (37 mg, 0.10 mmol), and a degassed 2 M (aq.) solution of K₃PO₄(50 μL, 0.1 mmol) were sequentially added under a stream of N₂. The reaction was sealed, flushed with N₂ for 5 min, and the reaction was heated to 65° C. for 24 h. The reaction was cooled to room temperature, filtered through MgSO₄ and CELITE, flushed with EtOAc (5×5 mL), and concentrated under reduced pressure. The crude residue was dissolved in DCM (1 mL) and TFA (93 μL, 1.2 mmol). The reaction was stirred at room temperature for 1 h. The reaction was concentrated under reduced pressure, and the crude residue was purified by reverse phase HPLC (MeCN/water with 0.05% NH₄HCOᵢ modifier) to provide 1-(1-(((4-((1R,5S)-3,8-diazabicyclo[3.2.1]octan-3-yl)-7-(5-chloro-6-methyl-1H-indazol-4-yl)-6,8-difluoroquinazolin-2-yl)oxy)methyl)-2,2-difluorocyclopropyl)-N,N-dimethylmethanamine (Ex. 64) as a racemic mixture. MS (ESI): m/z (M+H)⁺ 604. ¹H NMR (600 MHz, DMSO-d) δ 13.36 (s, 1H), 7.78-7.69 (m, 3H), 4.47 (d, J=11.7 Hz, 1H), 4.41 (d, J=11.3 Hz, 1H), 4.34-4.26 (m, 2H), 3.49 (m, 3H), 2.62 (m, 1H), 2.56 (s, 3H), 2.36 (dd, J=12.5, 5.9 Hz. 1H), 2.17 (s, 6H), 1.81-1.76 (m, 1H), 1.68 (m, 4H), 1.48 (s, 1H), 1.24 (s, 2H).

Examples in the table below were prepared using an analogous synthetic sequence to Ex. 64 above.

| | | | | SFC |
|---|---|---|---|---|
| | | | [M + H]⁺ | Separation |
| Ex | Structure | Compound Name | Found | Conditions |
| Ex 65 | 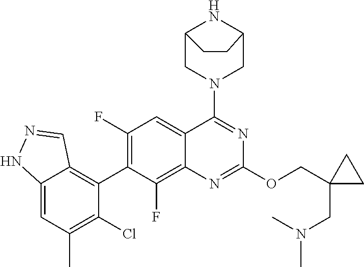 [SMILES: Cc1cc2[nH]ncc2c(-c2c(F)cc3c(N4CC5CCC(C4)N5)nc(OCC4(CN(C)C)CC4)nc3c2F)c1Cl] | 1-(1-(((4-((1R,5S)-3,8- diazabicyclo[3.2.1]octan- 3-yl)-7-(5-chloro-6- methyl-1H-indazol-4-yl)- 6,8-difluoroquinazolin-2- yl)oxy)methyl)cyclopropyl)- N,N-dimethylmethanamine | 568 | N/A |
| Ex 66a | 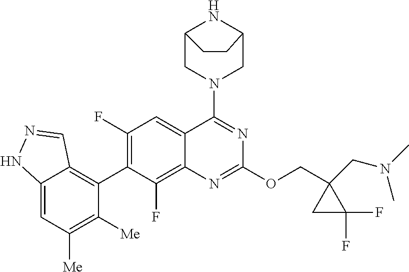 [SMILES: Cc1cc2[nH]ncc2c(-c2c(F)cc3c(N4CC5CCC(C4)N5)nc(OCC4(CN(C)C)CC4(F)F)nc3c2F)c1C] | 1-(1-(((4-(3,8-diazabicyclo [3.2.1]octan-3-yl)- 7-(5,6-dimethyl-1H- indazol-4-yl)-6,8- difluoroquinazolin-2- yl)oxy)methyl)-2,2- difluorocyclopropyl)-N,N- dimethylmethanamine | 584 | Column C; Conditions: 40% IPA w/ 0.1% NH₄OH |
| | Peak 1, SFC separation prior to Boc/THP | | | |
| | deprotection | | | |
| Ex 66b | 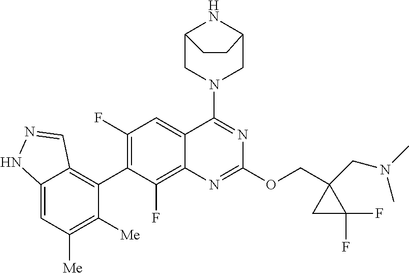 [SMILES: Cc1cc2[nH]ncc2c(-c2c(F)cc3c(N4CC5CCC(C4)N5)nc(OCC4(CN(C)C)CC4(F)F)nc3c2F)c1C] | 1-(1-(((4-(3,8-diazabicyclo [3.2.1]octan-3-yl)- 7-(5,6-dimethyl-1H- indazol-4-yl)-6,8- difluoroquinazolin-2- yl)oxy)methyl)-2,2- difluorocyclopropyl)-N,N- dimethylmethanamine | 584 | |
| | Peak 2, SFC separation prior to Boc/THP | | | |
| | deprotection | | | |

### Example 67: 6,8-difluoro-2-(((2S,4R)-4-fluoro-1-methylpyrrolidin-2-yl)methoxy)-7-(6-methyl-5-(trifluoromethyl)- H-indazol-4-yl)-4-((1S,5R)-1-methyl-3,8-diazabicyclo[3.2.1]octan-3-yl)quinazoline (Ex. 67)

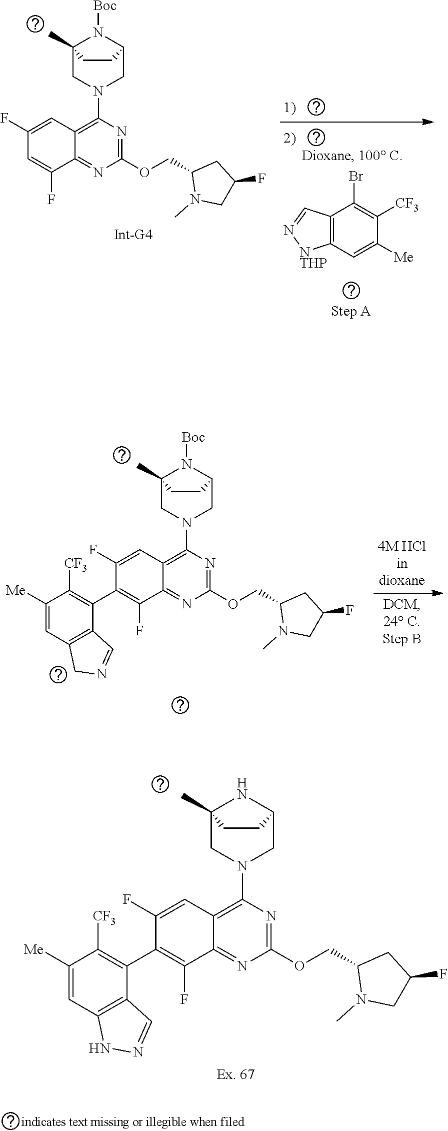

### Step A: tert-butyl (1S,5R)-3-(6,8-difluoro-2-(((2S4R)-4-fluoro-1-methylnpyrolidin-2-yl)methoxy)-7-(6-methyl-1-(tetrahydro-2H-pyran-2-yl)-5-(trifluoromethyl)-1H-indazol-4-yl)quinazolin-4-yl)-1-methyl-3,8-diazabicyclo[3.2.1]octane-8-carboxylate (Int-67a-1)

In the glovebox, TMP₂ZnMgCl₂LiCl (1406 μl, 0.211 mmol) was added to a 30 mL vial. In a separate vial, tert-butyl 3-(6,8-difluoro-2-(((2S,4R)-4-fluoro-1-methylpyrrolidin-2-yl)methoxy)quinazolin-4-yl)-1-methyl-3,8-diazabicyclo[3.2.1]octane-8-carboxylate (Int-G4) (100 mg, 0.192 mmol) was dissolved in THF (1 mL). The starting material solution was added dropwise to the zinc reagent. The starting material vessel was washed three times with THF (3×300 uL) and the combined rinse was added dropwise into the reaction vessel. The resulting mixture was stirred for 1 h at 24° C. In a third vial, 4-bromo-6-methyl-1-(tetrahydro-2H-pyran-2-yl)-5-(trifluoromethyl)-1H-indazole (Int-HHH5) (139 mg, 0.383 mmol) and CPhos Pd G4 (23.59 mg, 0.029 mmol) were dissolved in dioxane (1 mL). This solution was added dropwise into the reaction vessel. The vessel containing the electrophile was rinsed with dioxane (3×300 uL). The combined rinses were added dropwise into the reaction vessel. The reaction vessel was removed from the glovebox and then heated at 100° C. for 2h. The product mixture was cooled down to room temperature and diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with silica gel column, eluting with hexanes initially, grading to 100% ethyl acetate: ethanol=3:1, linear gradient to afford the desired product tert-butyl (1S,5R)-3-(6,8-difluoro-2-(((2S,4R)-4-fluoro-1-methylpyrrolidin-2-yl)methoxy)-7-(6-methyl-1-(tetrahydro-2H-pyran-2-yl)-5-(trifluoromethyl)-1H-indazol-4-yl)quinazolin-4-yl)-1-methyl-3,8-diazabicyclo[3.2.1]octane-8-carboxylate (Int-67a). This material was resolved by SFC (Column C, Conditions: 25% MeOH w/0.1% NH₄OH) to afford 4 peaks (2 atropisomers each with 2 THP stereocenters): tert-butyl (1S,5R)-3-(6,8-difluoro-2-(((2S,4R)-4-fluoro-1-methylpyrrolidin-2-yl)methoxy)-7-(6-methyl-1-(tetrahydro-2H-pyran-2-yl)-5-(trifluoromethyl)-1H-indazol-4-yl)quinazolin-4-yl)-1-methyl-3,8-diazabicyclo[3.2.1]octane-8-carboxylate (Int-67a-1, Peak 1). MS (ESI): m z (M+H)⁺ 804.

### Step B: 6.8-difluoro-2-(((2S,4R)-4-fluoro-1-methylpyrrolidin-2-ylmethoxy)-7-(6-methyl-5-(trifluoromethyl)-1H-indazol-4-yl)-4-((1S,5R)-1-methyl-3,8-diazabicyclo[3.2.1]octan-3-yl)quinazoline (Ex. 67)

tert-butyl (1S,5R)-3-(6,8-difluoro-2-(((2S,4R)-4-fluoro-1-methylpyrrolidin-2-yl)methoxy)-7-(6-methyl-1-(tetrahydro-2H-pyran-2-yl)-5-(trifluoromethyl)-1H-indazol-4-yl)quinazolin-4-yl)-1-methyl-3,8-diazabicyclo[3.2.1]octane-8-carboxylate (Int-67a-1) (7 mg, 8.71 μmol) was dissolved in DCM (363 μl). HCl in dioxane (72.6 μl) was added into the reaction vessel. The resulting mixture was stirred for 5h. The product mixture was diluted with DCM (5 mL) and saturated sodium bicarbonate aqueous solution (5 mL). The resulting mixture was stirred for 5 min. The resulting mixture was extracted five times with DCM (5×20 mL). The organic layer was dried over sodium sulfate. The dried solution was filtered and the filtrate was concentrated to dryness. The residue obtained was diluted with methanol (4 mL) and purified via preparative reverse phase HPLC (MeCN/water with 0.1% TFA modifier) to yield 6.8-difluoro-2-(((2S,41)-4-fluoro-1-methylpyrrolidin-2-yl)methoxy)-7-(6-methyl-5-(trifluoromethyl)-1H-indazol-4-yl)-4-((1S,5R)-1-methyl-3,8-diazabicyclo[3.2.1]octan-3-yl)quinazoline (Ex. 67). MS (ESI): m/z (M+H)⁺ 620, 1H NMR (499 MHz, DMSO-d₆) δ 9.41 (s, 2H), 7.89 (d, J=10.0 Hz, 1H), 7.80 (s, 1H), 7.70 (s, 1H), 5.48 (d, J=53.1 Hz, 1H), 4.86-4.79 (m, 1H), 4.68 (dd, J=12.7, 6.4 Hz, 1H), 4.46 (dd, J=23,3, 13.7 Hz, 2H), 4.23 (d, J=6.9 Hz, 2H), 3.76 (d, J=13.7 Hz, 1H), 3.66-3.46 (m, 2H), 3.06 (s, 3H), 2.68 (d, J=2.6 Hz. 3H). 2.59-2.53 (m, 1H), 2.40-2.05 (m, 2H), 1.96 (dd, J=26.2, 13.1 Hz, 1H), 1.85-1.71 (m, 1H), 1.52 (s, 3H).

Compounds in the table below were synthesized via a similar procedure to Ex. 67 with SFC separation in the penultimate step, unless otherwise indicated.

| | | | [M + H]⁺ | SFC Separation |
|---|---|---|---|---|
| Ex | Structure | Compound Name | Found | Conditions |
| Ex 68 | 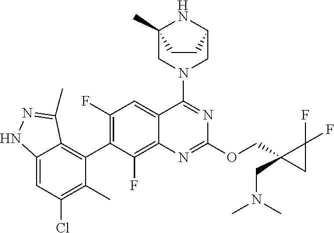 [SMILES: Cc1c(Cl)cc2[nH]nc(C)c2c1-c1c(F)cc2c(N3C[C@H]4CC[C@@](C)(C3)N4)nc(OC[C@@]3(CN(C)C)CC3(F)F)nc2c1F] | 1-((1R)-1-(((7-(6-chloro-3,5-dimethyl-1H- indazol-4-yl)-6,8-difluoro-4-((1S,5R)-1- methyl-3,8-diazabicyclo[3.2.1]octan-3-yl) quinazolin-2-yl)oxy)methyl)-2,2- difluorocyclopropyl)-N,N- dimethylmethanamine | 632 | Column L; Conditions: 25% MeOH w/0.1% NH₄OH; Peak 1 |
| | Derived from Peak 1 | | | |
| Ex 69 | 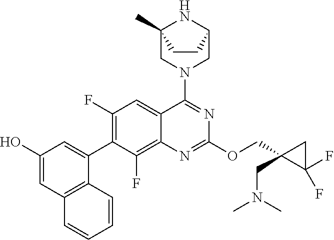 [SMILES: CN(C)C[C@@]1(COc2nc(N3C[C@H]4CC[C@@](C)(C3)N4)c3cc(F)c(-c4cc(O)cc5ccccc45)c(F)c3n2)CC1(F)F] | 4-(2-(((R)-1-((dimethylamino)methyl)-2,2- difluorocyclopropyl)methoxy)-6,8-difluoro- 4-((1S,5R)-1-methyl-3,8- diazabicyclo[3.2.1]octan-3-yl)quinazolin- 7-yl)naphthalen-2-ol | 596 | N/A |
| Ex 70 | 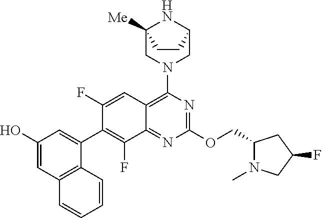 [SMILES: CN1C[C@H](F)C[C@H]1COc1nc(N2C[C@H]3CC[C@@](C)(C2)N3)c2cc(F)c(-c3cc(O)cc4ccccc34)c(F)c2n1] | 4-(6,8-difluoro-2-(((2S,4R)-4-fluoro-1- methylpyrrolidin-2-yl)methoxy)-4-((1S,5R)- 1-methyl-3,8-diazabicyclo[3.2.1]octan-3- yl)quinazolin-7-yl)naphthalen-2-ol | 564 | N/A |
| Ex 71a | 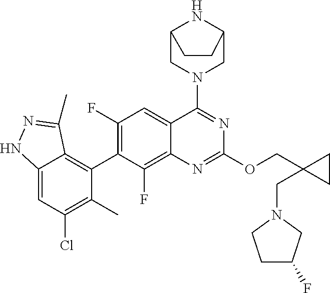 [SMILES: Cc1c(Cl)cc2[nH]nc(C)c2c1-c1c(F)cc2c(N3CC4CCC(C3)N4)nc(OCC3(CN4CC[C@@H](F)C4)CC3)nc2c1F] | 4-(3,8-diazabicyclo[3.2.1]octan-3-yl)-7-(6- chloro-3,5-dimethyl-1H-indazol-4-yl)-6,8- difluoro-2-((1-(((R)-3-fluoropyrrolidin-1- yl)methyl)cyclopropyl)methoxy)quinazoline | 626 | Column L; Conditions: 30% MeOH w/0.1% NH₄OH |
| | Derived from peaks 1/2 of Boc/THP- | | | |
| | protected material | | | |
| Ex 71b | 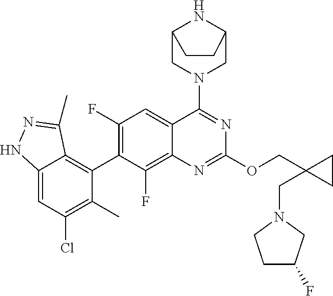 [SMILES: Cc1c(Cl)cc2[nH]nc(C)c2c1-c1c(F)cc2c(N3CC4CCC(C3)N4)nc(OCC3(CN4CC[C@@H](F)C4)CC3)nc2c1F] | 4-(3,8-diazabicyclo[3.2.1]octan-3-yl)-7-(6- chloro-3,5-dimethyl-1H-indazol-4-yl)-6,8- difluoro-2-((1-(((R)-3-fluoropyrrolidin-1- yl)methyl)cyclopropyl)methoxy)quinazoline | 626 | |
| | Derived from peaks 3/4 of Boc/THP- | | | |
| | protected material | | | |
| Ex 72 | 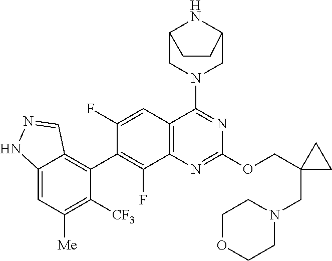 [SMILES: Cc1cc2[nH]ncc2c(-c2c(F)cc3c(N4CC5CCC(C4)N5)nc(OCC4(CN5CCOCC5)CC4)nc3c2F)c1C(F)(F)F] | 4-((1-(((4-(3,8-diazabicyclo[3.2.1]octan-3-yl)- 6,8-difluoro-7-(6-methyl-5-(trifluoromethyl)- 1H-indazol-4-yl)quinazolin-2- yl)oxy)methyl)cyclopropyl)methyl)morpholine | 644 | N/A |
| Ex. 125 | 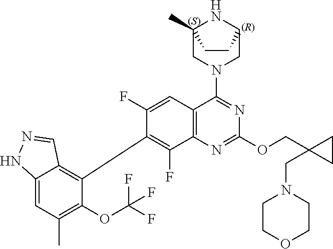 | 4-((1-(((6,8-difluoro-7-(6-methyl-5- (trifluoromethoxy)-1H-indazol-4-yl)-4- ((1S,5R)-1-methyl-3,8- diazabicyclo[3.2.1]octan-3-yl)quinazolin-2- yl)oxy)methyl)cyclopropyl)methyl) morpholine | 674.3 | NA |
| | Racemic mixture | | | |
| Ex. 126b | 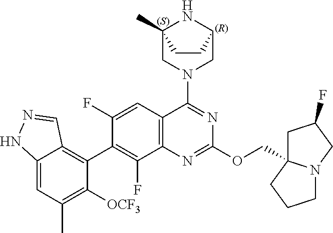 | 6,8-difluoro-2-(((2R,7aS)-2-fluorotetrahydro- 1H-pyrrolizin-7a(5H)-yl)methoxy)-7-(6- methyl-5-(trifluoromethoxy)-1H-indazol-4-yl)- 4-((1S,5R)-1-methyl-3,8-diazabicyclo [3.2.1]octan-3-yl)quinazoline | 662.3 | Column: Chiralpak IC-3 50 × 4.6 mm I.D., 3 um, Mobile phase: A: Hexane (0.1% DEA) B: IPA |
| | Peak 2. SFC at final step after | | | |
| | deprotection | | | |
| Ex. 126a | 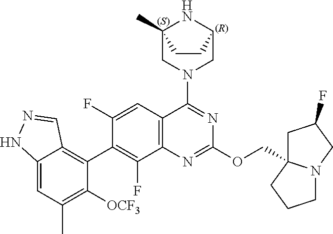 | 6,8-difluoro-2-(((2R,7aS)-2-fluorotetrahydro- 1H-pyrrolizin-7a(5H)-yl)methoxy)-7-(6- methyl-5-(trifluoromethoxy)-1H-indazol- 4-yl)-4-((1S,5R)-1-methyl-3,8-diazabicyclo [3.2.1]octan-3-yl)quinazoline | 662.3 | Column: Chiralpak IC-3 50 × 4.6 mm I.D., 3 um, Mobile phase: A: Hexane (0.1% DEA) B: IPA |
| | Peak 1, SFC separation after final | | | |
| | step | | | |
| Ex 73 | 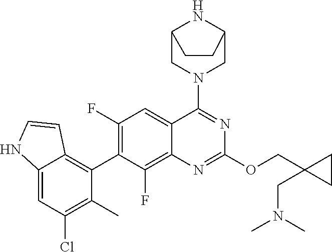 [SMILES: Cc1c(Cl)cc2[nH]ccc2c1-c1c(F)cc2c(N3CC4CCC(C3)N4)nc(OCC3(CN(C)C)CC3)nc2c1F] | 1-(1-(((4-(3,8-diazabicyclo[3.2.1]octan-3- yl)-7-(6-chloro-5-methyl-1H-indol-4-yl)- 6,8-difluoroquinazolin-2- yl)oxy)methyl)cyclopropyl)-N,N- dimethylmethanamine | 567 | N/A |
| | Negishi coupling run at 50° C. | | | |
| Ex 74 | 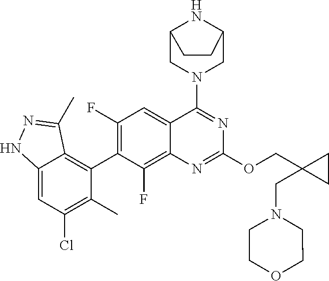 [SMILES: Cc1c(Cl)cc2[nH]nc(C)c2c1-c1c(F)cc2c(N3CC4CCC(C3)N4)nc(OCC3(CN4CCOCC4)CC3)nc2c1F] | 4-((1-(((4-(3,8-diazabicyclo[3.2.1]octan- 3-yl)-7-(6-chloro-3,5-dimethyl-1H- indazol-4-yl)-6,8-difluoroquinazolin-2- yl)oxy)methyl)cyclopropyl)methyl) morpholine | 624 | N/A |
| | Negishi coupling run at 50° C. | | | |
| Ex. 127 | 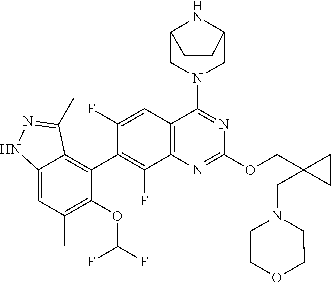 [SMILES: Cc1cc2[nH]nc(C)c2c(-c2c(F)cc3c(N4CC5CCC(C4)N5)nc(OCC4(CN5CCOCC5)CC4)nc3c2F)c1OC(F)F] | 4-((1-(((4-((1R,5S)-3,8- diazabicyclo[3.2.1]octan-3-yl)-7-(5- (difluoromethoxy)-3,6-dimethyl-1H- indazol-4-yl)-6,8-difluoroquinazolin-2- yl)oxy)methyl)cyclopropyl)methyl) morpholine | 656 | SFC final step Column A; Condition 0.1% NH3H2O ETOH |
| | Peak 1 | | | |
| Ex 75 | 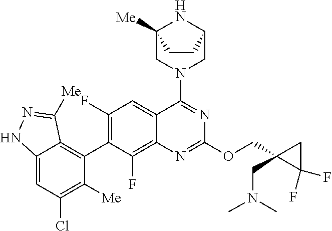 [SMILES: Cc1c(Cl)cc2[nH]nc(C)c2c1-c1c(F)cc2c(N3C[C@H]4CC[C@@](C)(C3)N4)nc(OC[C@]3(CN(C)C)CC3(F)F)nc2c1F] | 1-((1R)-1-(((7-(6-chloro-3,5-dimethyl-1H- indazol-4-yl)-6,8-difluoro-4-((1S,5R)-1- methyl-3,8-diazabicyclo[3.2.1]octan-3-yl) quinazolin-2-yl)oxy)methyl)-2,2- difluorocyclopropyl)-N,N- dimethylmethanamine | 632 | Column E; Conditions: 30% MeOH w/0.1% NH₄OH |
| | Peak 2, SFC separation at final step | | | |
| Ex 76a | 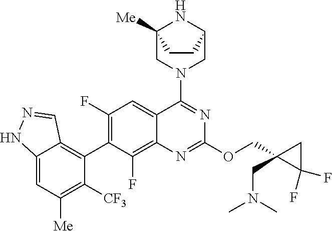 [SMILES: Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C(F)(F)F] | 1-((1R)-1-(((6,8-difluoro-7-(6-methyl-5- (trifluoromethyl)-1H-indazol-4-yl)-4- ((1S,5R)-1-methyl-3,8-diazabicyclo [3.2.1]octan-3-yl)quinazolin-2-yl) oxy)methyl)-2,2-difluorocyclopropyl)- N,N-dimethylmethanamine | 652 | Column E; Conditions: 25% MeOH w/0.1% NH₄OH |
| | Peak 1, SFC separation at final step | | | |
| Ex 76b | 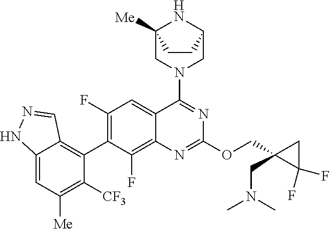 [SMILES: Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C(F)(F)F] | 1-((1R)-1-(((6,8-difluoro-7-(6-methyl-5- (trifluoromethyl)-1H-indazol-4-yl)-4- ((1S,5R)-1-methyl-3,8-diazabicyclo [3.2.1]octan-3-yl)quinazolin-2-yl) oxy)methyl)-2,2- difluorocyclopropyl)-N,N- dimethylmethanamine | 652 | |
| | Peak 2, SFC separation at final step | | | |
| Ex 77 | 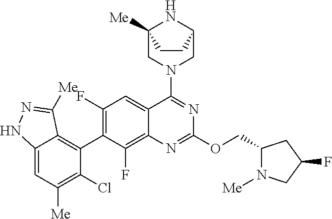 [SMILES: Cc1cc2[nH]nc(C)c2c(-c2c(F)cc3c(N4C[C@H]5CC[C@@](C)(C4)N5)nc(OC[C@@H]4C[C@@H](F)CN4C)nc3c2F)c1Cl] | 7-(5-chloro-3,6-dimethyl-1H-indazol-4-yl)- 6,8-difluoro-2-(((2S,4R)-4-fluoro-1- methylpyrrolidin-2-yl)methoxy)-4-((1S,5R)- 1-methyl-3,8-diazabicyclo[3.2.1]octan-3- yl)quinazoline | 600 | Column E; Conditions: 40% MeOH w/0.1% NH₄OH |
| | Peak 1, SFC separation at final step | | | |
| Ex 78 | 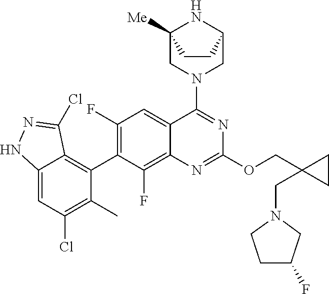 [SMILES: Cc1c(Cl)cc2[nH]nc(Cl)c2c1-c1c(F)cc2c(N3C[C@H]4CC[C@@](C)(C3)N4)nc(OCC3(CN4CC[C@@H](F)C4)CC3)nc2c1F] | 7-(3,6-dichloro-5-methyl-1H-indazol-4-yl)- 6,8-difluoro-2-((1-(((R)-3-fluoropyrrolidin-1- yl)methyl)cyclopropyl)methoxy)-4-((1S,5R)- 1-methyl-3,8-diazabicyclo[3.2.1]octan-3- yl)quinazoline | 660/662 | N/A |
| | Mixture of atropisomers | | | |
| Ex 79 | 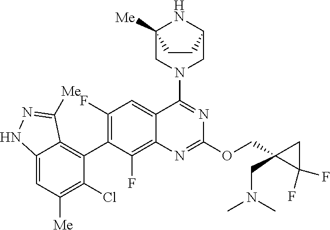 [SMILES: Cc1cc2[nH]nc(C)c2c(-c2c(F)cc3c(N4C[C@H]5CC[C@@](C)(C4)N5)nc(OC[C@]4(CN(C)C)CC4(F)F)nc3c2F)c1Cl] | 1-((1R)-1-(((7-(5-chloro-3,6-dimethyl-1H- indazol-4-yl)-6,8-difluoro-4-((1S,5R)- 1-methyl-3,8-diazabicyclo[3.2.1]octan- 3-yl)quinazolin-2-yl)oxy)methyl)-2,2- difluorocyclopropyl)-N,N- dimethylmethanamine | 632 | Column E; Conditions: 30% MeOH w/0.1% NH₄OH |
| | Peak 1, SFC separation at final step | | | |
| Ex 80a | 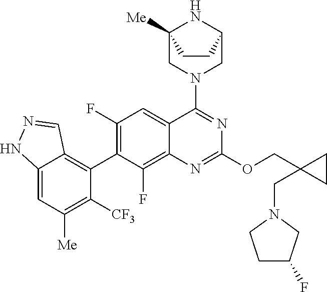 [SMILES: Cc1cc2[nH]ncc2c(-c2c(F)cc3c(N4C[C@H]5CC[C@@](C)(C4)N5)nc(OCC4(CN5CC[C@@H](F)C5)CC4)nc3c2F)c1C(F)(F)F] | 6,8-difluoro-2-((1-(((R)-3-fluoropyrrolidin-1- yl)methyl)cyclopropyl)methoxy)-7- (6-methyl-5-(trifluoromethyl)-1H-indazol- 4-yl)-4-((1S,5R)-1-methyl-3,8- diazabicyclo[3.2.1]octan-3- yl)quinazoline | 660 | Column E; Conditions: 30% MeOH w/0.1% NH₄OH |
| | Peak 1, SFC separation at final step | | | |
| Ex 80b | 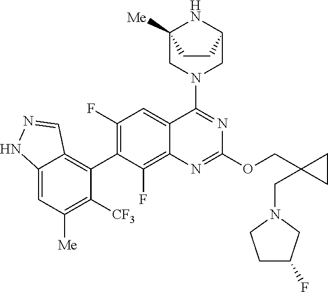 [SMILES: Cc1cc2[nH]ncc2c(-c2c(F)cc3c(N4C[C@H]5CC[C@@](C)(C4)N5)nc(OCC4(CN5CC[C@@H](F)C5)CC4)nc3c2F)c1C(F)(F)F] | 6,8-difluoro-2-((1-(((R)-3-fluoropyrrolidin-1- yl)methyl)cyclopropyl)methoxy)-7-(6- methyl-5-(trifluoromethyl)-1H-indazol- 4-yl)-4-((1S,5R)-1-methyl-3,8- diazabicyclo[3.2.1]octan-3- yl)quinazoline | 660 | |
| | Peak 2, SFC separation at final step | | | |
| Ex 81 | 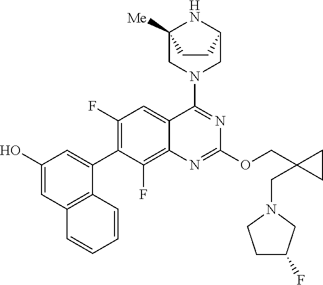 [SMILES: C[C@]12CC[C@H](CN(c3nc(OCC4(CN5CC[C@@H](F)C5)CC4)nc4c(F)c(-c5cc(O)cc6ccccc56)c(F)cc34)C1)N2] | 4-(6,8-difluoro-2-((1-(((R)-3- fluoropyrrolidin-1-yl)methyl)cyclopropyl) methoxy)-4-((1S,5R)-1-methyl-3,8- diazabicyclo[3.2.1]octan-3- yl)quinazolin-7-yl)naphthalen-2-ol | 604 | N/A |
| | TBS-protected naphthol starting | | | |
| | material | | | |
| Ex 82 | 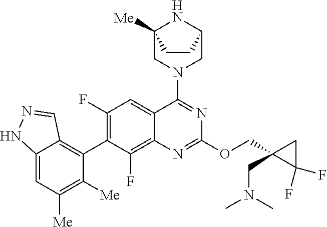 [SMILES: Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C] | 1-((1R)-1-(((7-(5,6-dimethyl-1H-indazol- 4-yl)-6,8-difluoro-4-((1S,5R)-1-methyl-3,8- diazabicyclo[3.2.1]octan-3-yl)quinazolin-2- yl)oxy)methyl)-2,2-difluorocyclopropyl)- N,N-dimethylmethanamine | 598 | N/A |
| | Mixture of atropisomers | | | |
| Ex 83 | 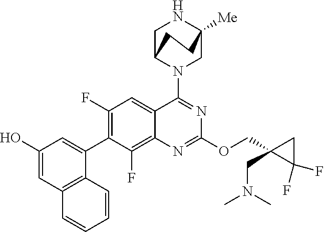 [SMILES: CN(C)C[C@@]1(COc2nc(N3C[C@]4(C)CC[C@H]3CN4)c3cc(F)c(-c4cc(O)cc5ccccc45)c(F)c3n2)CC1(F)F] | 4-(2-(((R)-1-((dimethylamino)methyl)-2,2- difluorocyclopropyl)methoxy)-6,8-difluoro- 4-((1S,4S)-4-methyl-2,5-diazabicyclo [2.2.2]octan-2-yl)quinazolin-7-yl) naphthalen-2-ol | 596 | N/A |
| | Mixture of atropisomers; TBS- | | | |
| | protected naphthol starting material | | | |
| Ex 84 | 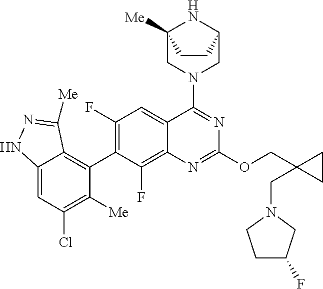 [SMILES: Cc1c(Cl)cc2[nH]nc(C)c2c1-c1c(F)cc2c(N3C[C@H]4CC[C@@](C)(C3)N4)nc(OCC3(CN4CC[C@@H](F)C4)CC3)nc2c1F] | 7-(6-chloro-3,5-dimethyl-1H-indazol- 4-yl)-6,8-difluoro-2-((1-(((R)-3- fluoropyrrolidin-1-yl)methyl)cyclopropyl) methoxy)-4-((1S,5R)-1-methyl-3,8- diazabicyclo[3.2.1]octan-3- yl)quinazoline | 640 | Column E; Conditions: 30% MeOH w/0.1% NH₄OH |
| | Peak 1, SFC separation at final step | | | |
| Ex 85 | 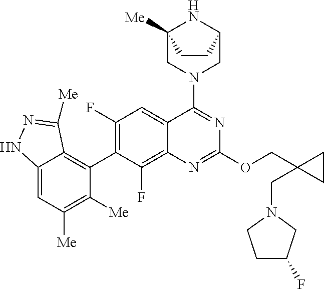 [SMILES: Cc1cc2[nH]nc(C)c2c(-c2c(F)cc3c(N4C[C@H]5CC[C@@](C)(C4)N5)nc(OCC4(CN5CC[C@@H](F)C5)CC4)nc3c2F)c1C] | 6,8-difluoro-2-((1-(((R)-3-fluoropyrrolidin- 1-yl)methyl)cyclopropyl)methoxy)-4- ((1S,5R)-1-methyl-3,8-diazabicyclo [3.2.1]octan-3-yl)-7-(3,5,6-trimethyl-1H- indazol-4-yl)quinazoline | 620 | Column E; Conditions: 35% MeOH w/0.1% NH₄OH |
| | Peak 1, SFC separation at final step | | | |
| Ex 86 | 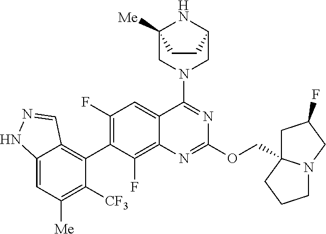 [SMILES: Cc1cc2[nH]ncc2c(-c2c(F)cc3c(N4C[C@H]5CC[C@@](C)(C4)N5)nc(OC[C@@]45CCCN4C[C@H](F)C5)nc3c2F)c1C(F)(F)F] | 6,8-difluoro-2-(((2R,7aS)-2-fluorotetrahydro- 1H-pyrrolizin-7a(5H)-yl)methoxy)-7- (6-methyl-5-(trifluoromethyl)-1H-indazol- 4-yl)-4-((1S,5R)-1-methyl-3,8-diazabicyclo [3.2.1]octan-3-yl)quinazoline | 646 | Column E; Conditions: 25% MeOH w/0.1% NH₄OH |
| | Peak 1, SFC separation at final step | | | |
| Ex. 128a | 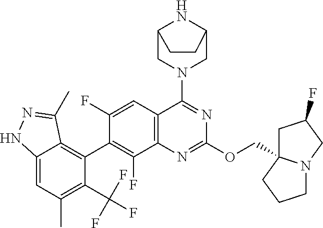 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1C(F)(F)F] | 4-((1R,5S)-3,8-diazabicyclo[3.2.1]octan-3- yl)-7-(3,6-dimethyl-5-(trifluoromethyl)-1H- indazol-4-yl)-6,8-difluoro-2-(((2R,7aS)-2- fluorotetrahydro-1H-pyrrolizin-7a(5H)-yl) methoxy)quinazoline | 646 | Column A; Condition: 0.1% NH3H2O ETOH |
| | Peak 1 | | | |
| Ex. 128b | 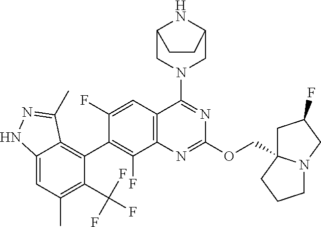 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1C(F)(F)F] | 4-((1R,5S)-3,8-diazabicyclo[3.2.1]octan-3- yl)-7-(3,6-dimethyl-5-(trifluoromethyl)-1H- indazol-4-yl)-6,8-difluoro-2-(((2R,7aS)-2- fluorotetrahydro-1H-pyrrolizin-7a(5H)-yl) methoxy)quinazoline | 646 | Column A; Condition: 0.1% NH3H20 ETOH |
| | Peak 2 | | | |
| Ex. 129a | 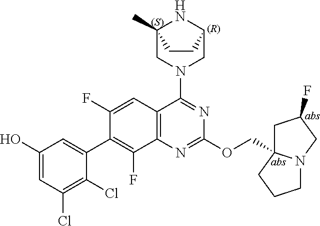 | 3,4-dichloro-5-(6,8-difluoro-2-(((2R,7aS)-2- fluorotetrahydro-1H-pyrrolizin-7a(5H)- yl)methoxy)-4-((1S,5R)-1-methyl-3,8- diazabicyclo[3.2.1]octan-3-yl)quinazolin-7- yl)phenol, HCl | 608 | Condition E; 30% MeOH w/0.1% NH₄OH |
| | Peak 1, SFC separation penultimate | | | |
| | to final step | | | |
| Ex. 129b | 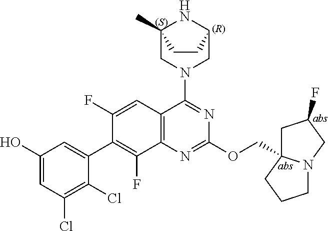 | 3,4-dichloro-5-(6,8-difluoro-2-(((2R,7aS)-2- fluorotetrahydro-1H-pyrrolizin-7a(5H)- yl)methoxy)-4-((1S,5R)-1-methyl-3,8- diazabicyclo[3.2.1]octan-3-yl)quinazolin-7- yl)phenol, HCl | 608.2 | Condition E; 30% MeOH w/0.1% NH₄OH |
| | Peak 2, SFC separation penultimate | | | |
| | to final step | | | |
| Ex. 130 | 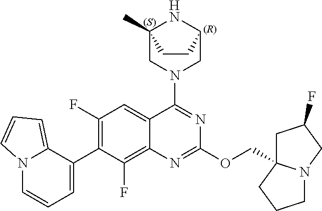 | 6,8-difluoro-2-(((2R,7aS)-2-fluorotetrahydro- 1H-pyrrolizin-7a(5H)-yl)methoxy)-7- (indolizin-8-yl)-4-((1S,5R)-1-methyl-3,8- diazabicyclo[3.2.1]octan-3-yl)quinazoline, 2TFA | 563.1 | -NA- |
| Ex. 131 | 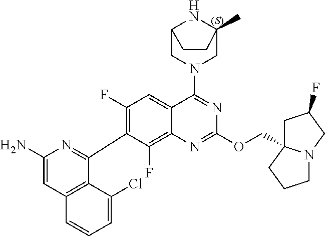 | 8-chloro-1-(6,8-difluoro-2-(((2S,7aR)-2- fluorotetrahydro-1H-pyrrolizin-7a(5H)-yl) methoxy)-4-((1S,5R)-1-methyl-3,8-diaza- bicyclo[3.2.1]octan-3-yl)quinazolin-7-yl) isoquinolin-3-amine | 624 | Column L; Condition 40% MeOH w/0.1% NH4OH |
| | Peak 1 from SFC | | | |
| Ex. 132 | 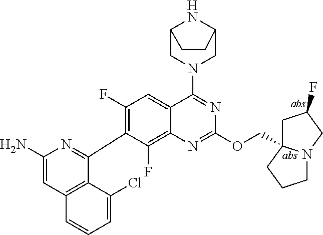 [SMILES: Nc1cc2cccc(Cl)c2c(-c2c(F)cc3c(N4CC5CCC(C4)N5)nc(OC[C@@]45CCCN4C[C@H](F)C5)nc3c2F)n1] | 1-(4-((1R,5S)-3,8-diazabicyclo[3.2.1]octan- 3-yl)-6,8-difluoro-2-(((2R,7aS)-2-fluoro tetrahydro-1H-pyrrolizin-7a(5H)-yl)methoxy) quinazolin-7-yl)-8-chloroisoquinolin-3- amine | 610 | Column: IB-N, 21 × 250 mm, 5 um; Condition 45% MeOH w/ 0.1% NH4OH |
| | Peak 2 from SFC | | | |
| Ex. 133a | 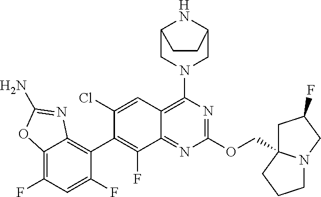 [SMILES: Nc1nc2c(-c3c(Cl)cc4c(N5CC6CCC(C5)N6)nc(OC[C@@]56CCCN5C[C@H](F)C6)nc4c3F)c(F)cc(F)c2o1] | 4-(4-((1R,5S)-3,8-diazabicyclo[3.2.1]octan- 3-yl)-6-chloro-8-fluoro-2-(((2R,7aS)-2- fluorotetrahydro-1H-pyrrolizin-7a(5H)- yl)methoxy)quinazolin-7- yl)-5,7-difluorobenzo[d]oxazol-2-amine | 618 | SFC before deprotection Column A; Condition 0.1% NH3H20 ETOH |
| | Peak 1 from SFC before deprotection | | | |
| Ex. 133b | 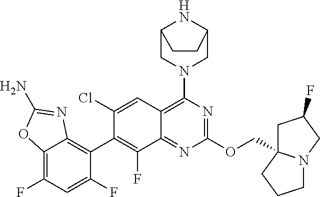 [SMILES: Nc1nc2c(-c3c(Cl)cc4c(N5CC6CCC(C5)N6)nc(OC[C@@]56CCCN5C[C@H](F)C6)nc4c3F)c(F)cc(F)c2o1] | 4-(4-((1R,5S)-3,8-diazabicyclo[3.2.1] octan-3-yl)-6-chloro-8-fluoro-2-(((2R,7aS)- 2-fluorotetrahydro-1H-pyrrolizin-7a(5H)- yl)methoxy)quinazolin-7-yl)-5,7- difluorobenzo[d]oxazol-2-amine | 618 | SFC before deprotection Column A; Condition 0.1% NH3H20 ETOH |
| | Peak 2 from SFC before deprotection | | | |
| | Suzuki with | | | |
| | CPhos Pd-G3 THF/50° C., 15 hr | | | |
| Ex. 134 | 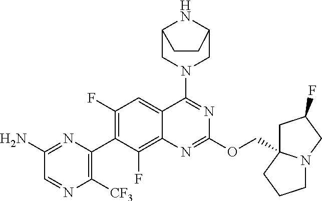 [SMILES: Nc1cnc(C(F)(F)F)c(-c2c(F)cc3c(N4CC5CCC(C4)N5)nc(OC[C@@]45CCCN4C[C@H](F)C5)nc3c2F)n1] | 6-(4-((1R,5S)-3,8-diazabicyclo[3.2.1] octan-3-yl)-6,8-difluoro-2-(((2R,7aS)-2- fluorotetrahydro-1H-pyrrolizin-7a(5H)- yl)methoxy)quinazolin- 7-yl)-5-(trifluoromethyl)pyrazin-2-amine | 595 | -NA- |
| | Mixture of atropisomers | | | |
| Ex. 135 | 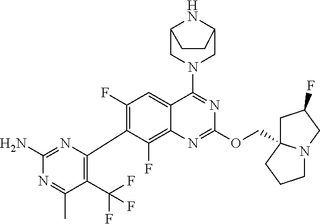 [SMILES: Cc1nc(N)nc(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8-diazabicyclo[3.2.1] octan-3-yl)-6,8-difluoro-2-(((2R,7aS)-2- fluorotetrahydro-1H-pyrrolizin-7a(5H)- yl)methoxy)quinazolin-7-yl)-6-methyl-5- (trifluoromethyl)pyrimidin-2- amine | 609 | -NA- |
| | Mixture of atropisomers | | | |

### Ex 136: Synthesis of 2-(((R)-1-((dimethylamino)methyl)-2,2-difluorocyclooronpyl)methoxy)-8-fluoro-7-(3-hydroxynaphthalen-1-yl)-4-((1S,5R)-1-methyl-3,8-diazabicyclo[3.2.1]octan-3-yl)quinazoline-6-carbonitrile (Ex. 136a and 136b)

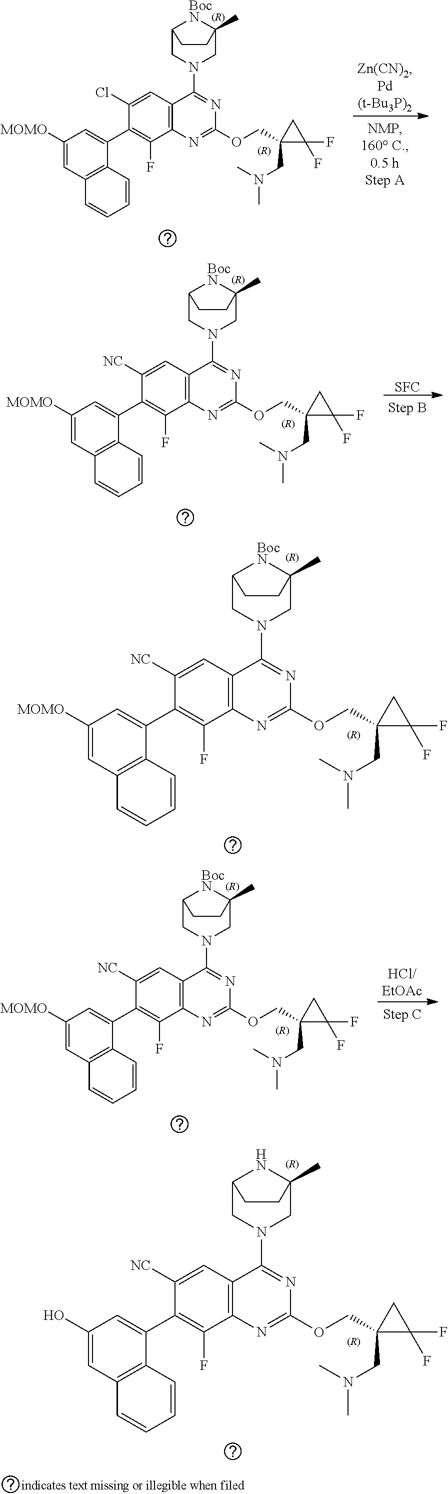

### Step A: tert-butyl (1S,5R)-3-(6-cyano-2-(((R)-1-((dimethylamino)methyl)-2,2-difluorocyclopropyl)methoxy)-8-fluoro-7-(3(methoxymethoxy)naphthalen-1-yl)quinazolin-4-yl)-1-methyl-3,8-diazabicyclo[3.2.1]octane-8-carboxylate (Int 136a)

To a solution of tert-butyl (1S,5R)-3-(6-chloro-2-(((R)-1-((dimethylamino)methyl)-2,2-difluorocyclopropyl)methoxy)-8-fluoro-7-(3-(methoxymethoxy)naphthalen-1-yl)quinazolin-4-yl)-1-methyl-3,8-diazabicyclo[3.2.1]octane-8-carboxylate (Intermediate from Ex 35—MOM/Boc protected) (120 mg, 0.159 mmol) in NMP (2 mL) was added zinc cyanide (150 mg, 1.278 mmol) and bis(tri-tert-butylphosphine)palladium(0) (40.5 mg, 0.079 mmol) at 25° C. under N₂. The mixture was stirred at 160° C. for 0.5 h under microwave and monitored by LCMS, The reaction mixture was quenched with water (15 mL), extracted with EtOAc (3×15 mL). The combined organic layer was washed with brine (3×10 mL), dried over Na₂SO₄, filtered and the solvent was evaporated under reduced pressure to give the crude which was purified by per-TLC (Pet, ether:EtOAc=1:1, basified with NH₃H₂O (1 mL) before chromatography), to give tert-butyl (1S,5R)-3-(6-cyano-2-(((R)-1-((dimethylamino)methyl)-2,2-difluorocyclopropyl)methoxy)-8-fluoro-7-(3-(methoxymethoxy)naphthalen-1-yl)quinazolin-4-yl)-1-methyl-3,8-diazabicyclo[3.2.1]octane-8-carboxylate. MS (ESI): m/z (M+H)⁺ 747.

### Step B: Tert-butyl (1S,5R)-3-(6-cyano-2-((R)-1-((dimethylamino)methyl)-2,2-difluorocyclopropyl)methoxy)-8-fluoro-7-(3-methoxymethoxy)naphthalen-1-yl)quinazolin-4-yl)-1-methyl-3,8-diazabicyclo[3.2.1]octane-8-carboxylate (Int-136-3A and Int-136-3B)

The racemic mixture of tert-butyl (1S,5R)-3-(6-cyano-2-(((R)-1-((dimethylamino)methyl)-2,2-difluorocyclopropyl)methoxy)-8-fluoro-7-(3-(methoxymethoxy)naphthalen-1-yl)quinazolin-4-yl)-1-methyl-3,8-diazabicyclo[3.2.1]octane-8-carboxylate (Int-136a) (90 mg, 0.120 mmol) was separated by preparative SFC (Column: C: Condition: 0.1% NNHH₂O EtOH Begin B 45 End B 45 Gradient Time (min) 100% B Hold Time (min) FlowRate (mL/min) 80) to give tert-butyl (1S,5R)-3-(6-cyano-2-(((R)-1-((dimethylamino)methyl)-2,2-difluorocyclopropyl)methoxy)-8-fluoro-7-(3-(methoxymethoxy)naphthalen-1-yl)quinazolin-4-yl)-1-methyl-3,8-diazabicyclo[3.2.1]octane-8-carboxylate (Int-136-3A), the first eluting isomer from SFC) and tert-butyl (1S,5R)-3-(6-cyano-2-(((R)-1-((dimethylamino)methyl)-2,2-difluorocyclopropyl)methoxy)-8-fluoro-7-(3-(methoxymethoxy)naphthalen-1-yl)quinazolin-4-yl)-1-methyl-3,8-diazabicyclo[3.2.1]octane-8-carboxylate (Int-136-3B), the second eluting isomer from SFC). MS (ESI): m/Z (M+H)⁺ 747.

### Step C: 2-(((R)-1-((dimethylamino)methyl)-2,2-difluorocycloyroyl)methoxy)-8-fluoro-7-(3-hydroxynaphthalen-1-yl)-4-((S,5R)-4-methyl-3,8-diazabicyclo[3.2.1]octan-3-yl)quinazoline-6-carbonitrile (Ex. 136-Peak 1)

A solution of tert-butyl (1S,5R)-3-(6-cyano-2-(((R)-1-((dimethylamino)methyl)-2,2-difluorocyclopropyl))methoxy)-8-fluoro-7-(3-(methoxymethoxy)naphthalen-1-yl)quinazolin-4-yl)-1-methyl-3,8-diazabicyclo[3.2.1]octane-8-carboxylate (Int-136-3A) (29 mg, 0.039 mmol) (3A) in HCl/EtOAc (0.1 mL, 1.218 mmol) (4 M in EtOAc) was stirred at 25° C. for 0.5 h. The reaction was monitored by LCMS, The solvent was evaporated under reduced pressure to give the crude which was purified by preparative HPLC (Column: Waters Xbridge BEH C18 100*25 mm*5 um Condition: water (0.225% FA)-ACN Begin B 0 End B 25 Gradient Time (min) 12 100% B Hold Time (min) 2 FlowRate (mL/min) 25) to give 2-(((R)-1-((dimethylamino)methyl)-2,2-difluorocyclopropyl)methoxy)-8-fluoro-7-(3-hydroxynaphthalen-1-yl)-4-((1S,5R)-1-methyl-3,8-diazabicyclo[3.2.]octan-3-yl)quinazoline-6-carbonitrile. Peak 1 (Ex. 136-A): ¹H NMR (400 MHz, MeOD) δ 8.35 (s, 1H), 7.78 (d, J=8.2 Hz, 1H), 7.44 (t, J=7.6 Hz, 1H), 7.35-7.27 (m, 2H), 7.26-7.19 (m, 1H), 7.11 (d, J=2,3 Hz, 1H), 4.73-4.55 (m, 4H), 4.07 (br d, J=5.9 Hz, 1H), 3.84 (br d, J=13.3 Hz, 1H), 3.71 (br d, J=13.7 Hz, 1H), 3.25 (br d, J=13.7 Hz, 1H), 2.97 (br d, J=13.7 Hz, 1H), 2.63 (s, 6H), 2.20-2.04 (m, 2H), 2.02-1.92 (m, 1H), 1.89-1.71 (m, 2H), 1.67-1.57 (m, 1H), 1.52 (s, 3H). MS (ESI): m/z (M+H)⁺ 603. The procedure of Peak 2 is similar as Peak 1 using Int-136-3B, and the spectrum data is as below: MS (ESI): m¹z (M+H)⁺ 603. Peak 2 (Ex. 136-B): ¹H NMR (400 MHz, MeOD) δ 8.36 (s, 1H), 7.78 (d, 7J=8.2 Hz, 1H), 7.44 (t, J=7.4 Hz, 1H), 7.35-7.27 (m, 2H), 7.26-7.20 (m, 1H), 7.13 (s, 1H), 4.77-4.53 (m, 4H), 4.06 (br d, J=5.9 Hz, 1H), 3.84 (br d, J=13.3 Hz, 1H), 3.67 (br d, J=13.3 Hz, 1H), 3.20 (br d, J=:13.3 Hz, 1H), 2.90 (br d, J=13.3 Hz, 1H), 2.58 (s, 6H), 2.23-1.94 (m, 3H), 1.88-1.70 (m, 2H), 1.64-1.55 (m, 1H), 1.51 (s, 3H).

### Example 87: 64443,8-diazabicyclo[3.2.1]octan-3-yl)-6,8-difluoro-2-((1-(morpholinomethyl)cyclopropyl)methoxy)quinazolin-7-yl)-4-methyl-5-(trifluoromethyl)pyndin-2-amine (Ex. 87)

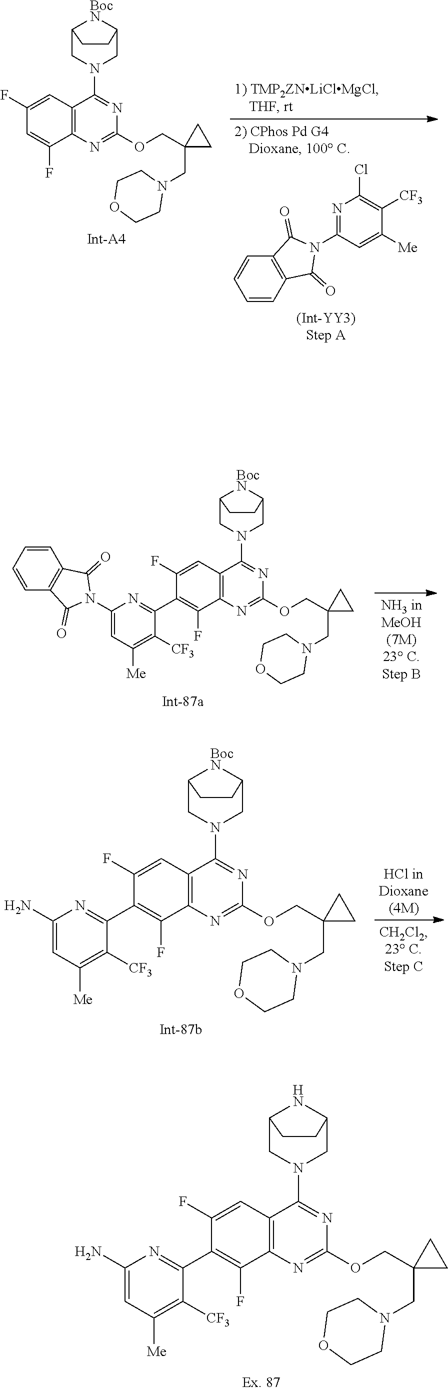 [SMILES: CC(C)(C)OC(=O)N1C2CCC1CN(c1nc(OCC3(CN4CCOCC4)CC3)nc3c(F)cc(F)cc13)C2 ; Cc1cc(N2C(=O)c3ccccc3C2=O)nc(Cl)c1C(F)(F)F ; Cc1cc(N2C(=O)c3ccccc3C2=O)nc(-c2c(F)cc3c(N4CC5CCC(C4)N5C(=O)OC(C)(C)C)nc(OCC4(CN5CCOCC5)CC4)nc3c2F)c1C(F)(F)F ; Cc1cc(N)nc(-c2c(F)cc3c(N4CC5CCC(C4)N5C(=O)OC(C)(C)C)nc(OCC4(CN5CCOCC5)CC4)nc3c2F)c1C(F)(F)F ; Cc1cc(N)nc(-c2c(F)cc3c(N4CC5CCC(C4)N5)nc(OCC4(CN5CCOCC5)CC4)nc3c2F)c1C(F)(F)F]

### Step A: tert-butyl 34746-(1,3-dioxoisoindolin-2-yl)-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87a)

TMP₂Zn·LiCl·MgCl (1008 μl. 0.151 mmol, 0.15 M) was added to a microwave vial under Ar. In a separate vial was added tert-butyl 3-(6,8-difluoro-2-((1-(morpholinomethyl)cyclopropyl)methoxy)quinazolin-4-yl)-3,8-diazabicyclo[3.2.1]octane-8-carboxylate (Int-A4) (75 mg, 0.137 mmol) and THF (0.5 mL). The mixture was sonicated until the solution became homogeneous and was then added to the microwave vial. The mixture was stirred for 45 minutes at room temperature. After 45 minutes, a mixture of 2-(6-chloro-4-methyl-5-(trifluoromethyl)pyridin-2-yl)isoindoline-1,3-dione (Int-YY3) (94 mg, 0.275 mmol) in dioxane and a mixture of CPhosPd G4 (16.91 mg, 0.021 mmol) in dioxane (0.5 mL) were added to the vial and heated to 100° C. for 2 hours. Upon cooling to room temperature, the mixture was diluted with ethyl acetate and saturated aqueous ammonium chloride. The organic layer was separated, dried over magnesium sulfate, filtered and concentrated under reduced pressure. The resulting residue was purified by silica gel column chromatography (0-50% 3:1 ethyl acetate: ethanol gradient in hexanes) to afford tert-butyl 3-(7-(6-(1,3-dioxoisoindolin-2-yl)-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87a). MS (EST): m/z (M+H)⁺ 850.

### Step B: tert-butyl 3-(7-(6-amino-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87b)

To a flask containing tert-butyl 3-(7-(6-(1,3-dioxoisoindolin-2-yl)-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87a) (30 mg, 0.035 mmol) was added ammonia in methanol (101 μl, 0.706 mmol). The mixture was stirred overnight. The mixture was concentrated to yield tert-butyl 3-(7-(6-amino-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87b) and was taken on to the next step without further purification. MS (ESI): m/z (M+H)⁺ 720.

### Step C: 6-(4-(3.8-diazabicyclo[3.2.1]octan-3-yl)-6,8-difluoro-2-((1-(morpholinomethyl)cyclopropyl)methoxy)quinazolin-7-yl)-4-methyl-5-(trifluoromethyl)pyridin-2-amine (Ex. 87)

To a flask containing tert-butyl 3-(7-(6-amino-4-methyl-3-(trifluoromethyl)pyridin-2-yl)-6,8-difluoro-2-((1-(morpholinomethyl)cyclopropyl)methoxy)quinazolin-4-yl)-3,8-diazabicyclo[3.2.1]octane-8-carboxylate (Int-87b) (21 mg, 0.029 mmol) were added DCM (1 mL) and then HCl in dioxane (0.073 mL, 0.292 mmol, 4 M). The mixture was stirred overnight at room temperature. The mixture was concentrated under reduced pressure and purified by preparative reverse phase HPLC (MeCN/water with 0.1% TFA modifier) to afford 6-(4-(3.8-diazabicyclo[3.2.1]octan-3-yl)-6,8-difluoro-2-((1-(morpholinomethyl)cyclopropyl)methoxy)quinazolin-7-yl)-4-methyl-5-(trifluoromethyl)pyridin-2-amine (Ex. 87). MS (ESI): m/z (M+H)⁺ 620, 1H NMR (500 MHz, DMSO-<d₆) δ 9.47 (s, 1H), 9.28 (d, J=8.9 Hz, 1H), 9.05 (s, 1H), 7.71 (d, J=10.2 Hz, 1H), 6.87 (s, 1H), 6.52 (s, 1H), 4.52-4.43 (m, 1H), 4.38-4.28 (m, 2H), 4.25-4.21 (m, 1H), 4.21-4.14 (m, 2H), 4.05-3.98 (m, 2H), 3.79-3.67 (m, 4H), 3.66-3.59 (m, 2H), 3.32-3.23 (m, 2H), 3.19-3.07 (m, 2H), 2.38 (s, 3H), 2.09-1.90 (m, 4H), 0.92-0.85 (m, 2H), 0.83-0.75 (m, 2H).

Examples in the table below were prepared via an analogous reaction sequence as for Ex. 87 above.

| | | | [M + H]⁺ | |
|---|---|---|---|---|
| Ex | Structure | Compound Name | Found | SFC Conditions |
| Ex 88 | 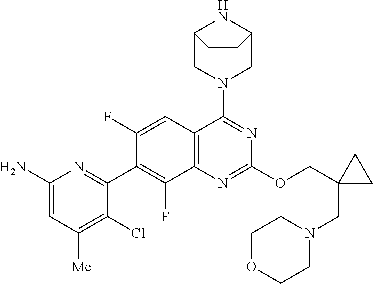 [SMILES: Cc1cc(N)nc(-c2c(F)cc3c(N4CC5CCC(C4)N5)nc(OCC4(CN5CCOCC5)CC4)nc3c2F)c1Cl] | 6-(4-((1R,5S)-3,8-diazabicyclo[3.2.1]octan-3-yl)-6,8- difluoro-2-((1- (morpholinomethyl)cyclopropy)methoxy)quinazolin- 7-yl)-5-chloro-4-methylpyridin-2-amine | 586 | |
| Ex. 137a | 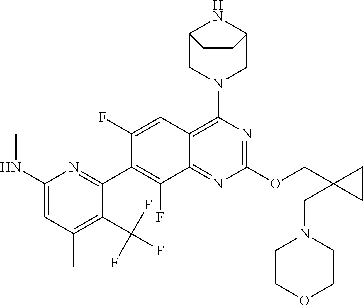 [SMILES: CNc1cc(C)c(C(F)(F)F)c(-c2c(F)cc3c(N4CC5CCC(C4)N5)nc(OCC4(CN5CCOCC5)CC4)nc3c2F)n1] | 6-(4-((1R,5S)-3,8-diazabicyclo[3.2.1]octan-3-yl)-6,8- difluoro-2-((1- (morpholinomethyl)cyclopropyl)methoxy)quinazolin- 7-yl)-N,4-dimethyl-5-(trifluoromethyl)pyridin-2- amine | 634.3 | Chiralpak AD-3 150ᵢ Á 4.6 mm I.D., 3 um Mobile phase: A: CO2 B: ethanol (0.05% DEA) |
| | Peak 1 | | | |
| | SFC resolution after Negishi, before | | | |
| | deprotection | | | |
| Ex. 137b | 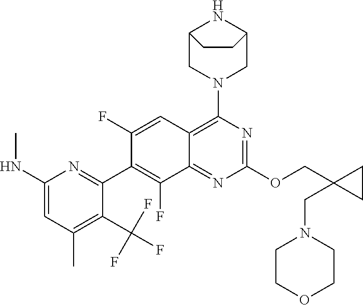 [SMILES: CNc1cc(C)c(C(F)(F)F)c(-c2c(F)cc3c(N4CC5CCC(C4)N5)nc(OCC4(CN5CCOCC5)CC4)nc3c2F)n1] | 6-(4-((1R,5S)-3,8-diazabicyclo[3.2.1]octan-3-yl)-6,8- difluoro-2-((1- morpholinomethyl)cyclopropyl)methoxy)quinazolin- 7-yl)-N,4-dimethyl-5-(trifluoromethyl)pyridin-2- amine Peak 2 | 634.3 | Chiralpak AK-3 150ᵢ Á 4.6 mm I.D., 3 um Mobile phase: A: CO2 B: ethanol (0.05% DEA) |
| | Peak 2 | | | |
| | SFC resolution after Negishi, before | | | |
| | final deprotection | | | |
| Ex. 138 | 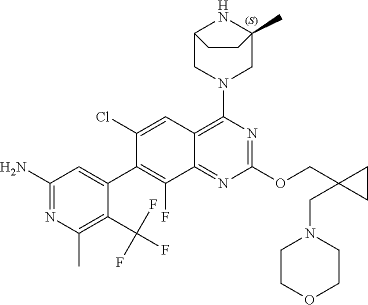 | 4-(6-chloro-8-fluoro-4-((1S,5R)-1-methyl-3,8- diazabicyclo[3.2.1]octan-3-yl)-2-((1- (morpholinomethyl)cyclopropyl)methoxy)quinazolin- 7-yl)-6-methyl-5-(trifluorometh- yl)pyridin-2-amine | 651.3 | Chiralcel OJ-3 100ᵢ Á 4.6 mm I.D., 3 um Mobile phase: A: CO2 B: ethanol (0.05% DEA) |
| | SFC resolution after Negishi, efore | | | |
| | final deprotection | | | |
| Ex. 139a | 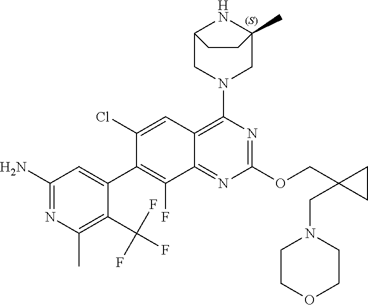 | 4-(6,8-difluoro-4-((1S,5R)-1-methyl-3,8- diazabicyclo[3.2.1]octan-3-yl)-2-((1-(morpho- linomethyl)cyclopropy)methoxy)quinazolin-7-yl)- 6-methyl-5-(trifluoromethyl)pyri- din-2-amine | 634 | Column Chiralcel OJ-3 150ᵢ Á 4.6 mm I.D., 3 um Mobile phase: A: CO2 B: methanol (0.05% DEA) |
| | Peak 1-SFC separation after | | | |
| | Negishi coupling before | | | |
| | deprotection | | | |
| Ex. 139b | 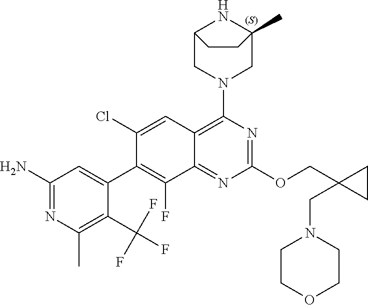 | 4-(6,8-difluoro-4-((1S,5R)-1-methyl-3,8- diazabicyclo[3.2.1]octan-3-yl)-2-((1-(morpho- linomethyl)cyclopropyl)methoxy)quinazolin-7-yl)- 6-methyl-5-(trifluoromethyl)pyri- din-2-amine | 634 | Column Chiralcel OJ-3 150ᵢ Á4.6 mm I.D., 3 um Mobile phase: A: CO2 B: methanol (0.05% DEA) |
| | Peak 2 - SFC separation after | | | |
| | Negishi coupling before | | | |
| | deprotection | | | |

### Intermediate (Int 140b_P2): 6.8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazlin4o (Peak 2)

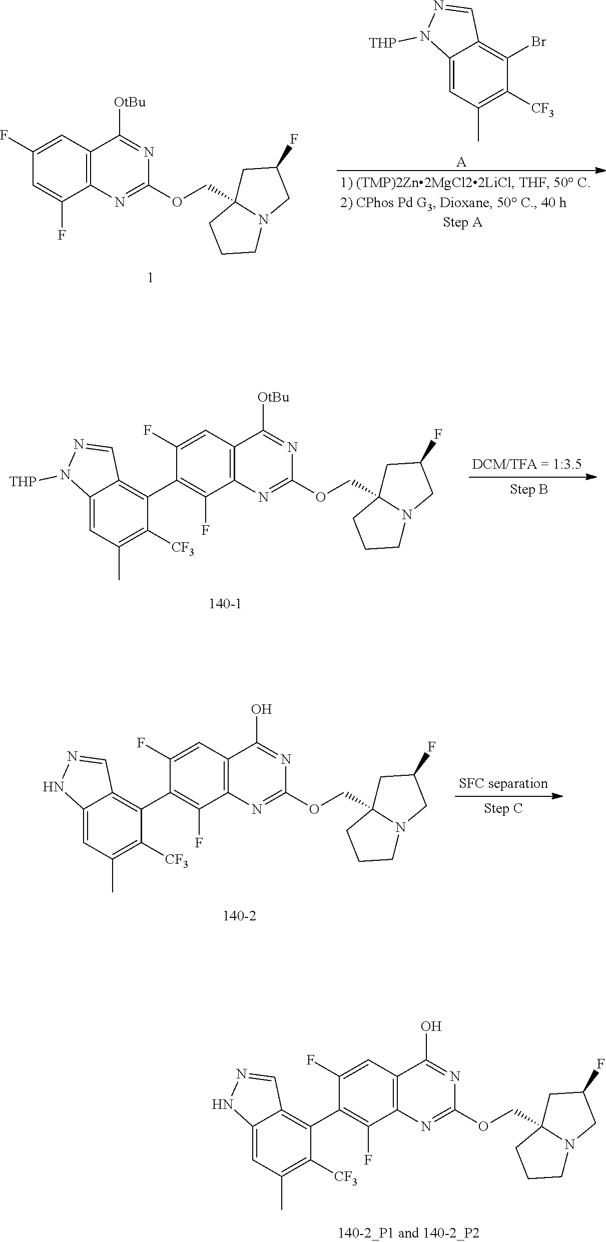

### Step A: 4-(tert-butoxy-6,8-difluoro-2-(((2R,7aS)-2-fluorotetrahydro-1H-pyrolizin-7a(5H)-yl)methoxy)-7-(6-methyl-1-(tetrahydro-2H-pyran-2-yl)-5-(trifluoromethyl)-1H-indazol-4-yl)quinazoline (Int-140-1)

To 4-(tert-butoxy)-68-difluoro-2-(((2R,7aS)-2-fluorotetrahydro- H-pyrrolizin-7a(5H)-yl)methoxy)quinazoline (Int-A110) (5 g, 12.64 mmol) was added (TMP)₂Zn 2MgCl—LiCl (115 mL, 37.9 mmol) (0.33 M in THF) at 25 C under N₂ atmosphere, and the mixture was stirred at 50° C. for 2 h. The reaction was monitored by LCMS that showed the exchange is completed (quenched with 12 (dissolved in THF)). Then to the reaction mixture was added a solution of 4-bromo-6-methyl-1-(tetrahydro-2H-pyran-2-yl)-5-(trifluoromethyl)-1H-indazole (Int-HHH5) (6.89g 18.97 mmol) and CPhos Pd G3 (1.020 g, 1.264 mmol) in 1.4-dioxane (30 mL) at 25° C. A total of 10 bottles were used for the reaction (500 mg each batch). The reaction was stirred at 50° C. for 40 h and monitored by LCMS, The reaction mixture was diluted with EtOAc (100 mL) and saturated NaHCO₃ solution (50 mL) was added to the mixture. The mixture was filtered and dried over Na2SO₄, then the solvent was evaporated under reduced pressure to give the crude product. The crude product was purified by flash silica gel chromatography (ISCO*: 120 g SepaFlash® Silica Flash Column, Eluent of 33% ethyl acetate in petroleum ether gradient to give 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline. MS (ESI): m/z (M+H)⁺ 678.

### Step B: 6.8-difluoro-2-(((2R,7aS)-2-fluorotetrahydro-1H-pyrolizin-7a(5H)-yl)methoxy)-7-(6-methyl-5-(trifluoromethyl)-1H-indazol-4-yl)quinazolin-4-ol (Int-140-2)

To a solution of TFA (9 mL, 117 mmol) in DCM (30 mL) was added 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline (Int-140-1) (4 g, 5.90 mmol) at 25° C. The reaction was stirred at 25° C. for 6 h. The solvent was evaporated under reduced pressure to give the crude which was washed by saturated aqueous NaHCO₃(3×20 mL), the combined organic phase was dried over anhydrous Na₂SO₄, filtered and concentrated in vacuo to give 6.8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol which was used in the next step directly without purification. MS (ESI): m/z (M+H)⁺ 538.

### Step. C: Separation via SFC to give 6.8-difluoro-2-(((2R7aS)-2-fluorotetrahydro-1H-pyrrolizin-7a(5H)-yl)methoxy)-7-(6-methyl-5-(trifluoromethyl)-1H-indazol4-yl)quinazolin-4-ol (Int-140-2 P1 and Int-140-2 P2 (Peak 2)

The racemic 6.8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (3.10 g, 5.77 mmol) was separated by preparative SFC (Column: DAICEL CHIRALPAK AD (250 mm×50 mm, 10 um) Condition 0.1% NH₃H₂O EtOH, followed by preparative HPLC (Column: Waters Xbridge BEH C18 100×25 mm×5 um Condition water (FA)-ACN to give 6.8-difluoro-2-(((2R,7aS)-2-fluorotetrahydro-1H-pyrroliin-7a(5H)-yl)methoxy)-7-(6-methyl-5-(trifluoromethyl)-1H-indazol-4-yl)quinazolin-4-ol (Int-140-2_P1, the first eluting isomer from SFC) and 6.8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-140-2_P2, the second eluting isomer from SFC). MS (ESI): m/z (M+H)⁺ 538, 140-2_P.: ¹H NMR (400 MHz, METHANOL-d₆)=7.76 (d, J=8.8 Hz, 1H), 7.68 (s, 1H), 7.59 (s, 1H), 5.46 (d, J=13.2 Hz, 1H), 4.53-4.40 (m, 2H), 3.63-3.45 (m, 3H), 3.22-3.20 (m, 1H), 2.71 (s, 3H), 2.53-2.33 (m, 2H), 2.30-2.20 (m, 1H), 2.18-2.09 (m, 2H), 2.05-1.94 (m, 1H). 140-2_P2: ¹H NMR (400MH., METHANOL-d₄) 6=7.75 (d, J=8.8 Hz, 1H), 7.68 (s, 1H), 7.59 (s, 1H), 5.46 (d, J=13.2 Hz, 1H), 4.50-4.36 (m, 2H), 3.63-3.40 (m, 3H), 3.17-3.15 (m, 1H), 2.70 (s, 3H), 2.47-2.28 (m, 2H), 2.25-2.16 (m, 1H), 2.15-2.04 (m, 2H), 2.02-1.90 (m, 1H).

Ex 140 to 162 in the table below were synthesized using the above intermediate and/or similar procedures with intermediates described earlier.

| | | | [M + H]⁺ | |
|---|---|---|---|---|
| Ex | Structure | Compound Name | Found | SFC Condition |
| Ex. 140 | 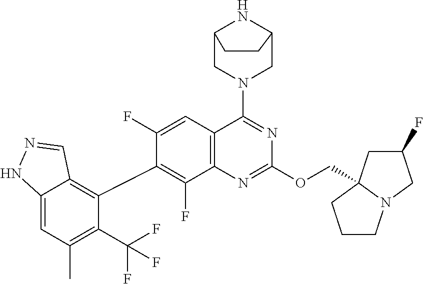 [SMILES: Cc1cc2[nH]ncc2c(-c2c(F)cc3c(N4CC5CCC(C4)N5)nc(OC[C@@]45CCCN4C[C@H](F)C5)nc3c2F)c1C(F)(F)F] | 4-((1R,5S)-3,8-diazabicyclo[3.2.1]octan-3-yl)-6,8-difluoro-2- (((2R,7aS)-2-fluorotetrahydro-1H-pyrrolizin-7a(5H)-yl)meth- oxy)-7-(6-methyl-5-(trifluoromethyl)-1H-indazol-4-yl)quin- azoline, TFA | 632 | |
| Ex. 141 | 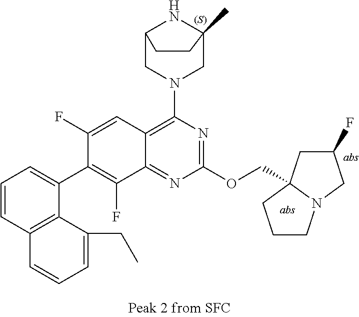 | 7-(8-ethylnaphthalen-1-yl)-6,8-difluoro-2-(((2R,7aS)-2- fluorotetrahydro-1H-pyrrolizin-7a(5H)-yl)methoxy)-4- ((1S,5R)-1-methyl-3,8-diazabicyclo[3.2.1]octan-3-yl)- quinazoline, TFA | 602 | |
| Ex. 142 | 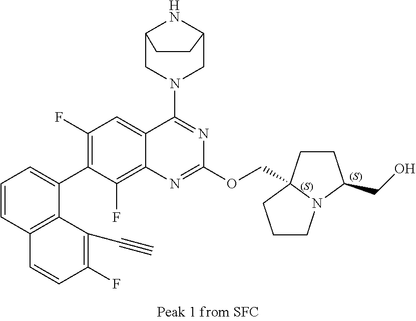 | ((3S,7aS)-7a-(((4-((1R,5S)-3,8-diazabicyclo[3.2.1]octan-3- yl)-7-(8-ethynyl-7-fluoronaphthalen-1-yl)-6,8-difluoroquin- azolin-2-yl)oxy)methyl)hexahydro-1H-pyrrolizin-3-yl)- methanol | | Column: Waters XSELECT C18 150 × 30 mm × 5 um; Condition: water (0.1%TFA)-MeCN |
| Ex. 143b | 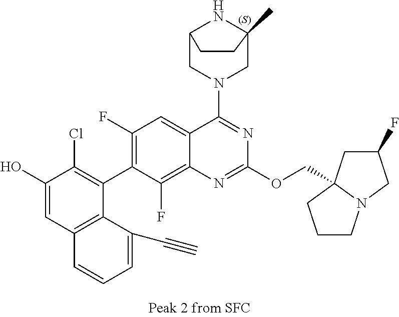 | 3-chloro-4-(6,8-difluoro-2-(((2R,7aS)-2-fluorotetrahydro- 1H-pyrrolizin-7a(5H)-yl)methoxy)-4-((1S,5R)-1-methyl- 3,8-diazabicyclo[3.2.1]octan-3-yl)quinazolin-7-yl)-5- ethynylnaphthalen-2-ol, 2TFA | 648 | Column M; Condition: 40% MeOH w/0.1% NH4OH |
| Ex. 143a | 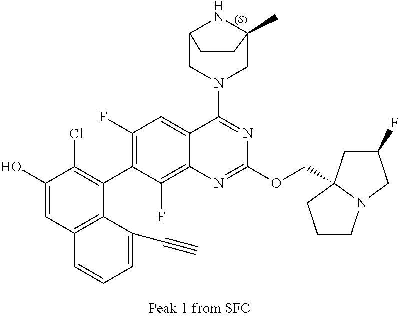 | 3-chloro-4-(6,8-difluoro-2-(((2R,7aS)-2-fluorotetrahydro- 1H-pyrrolizin-7a(5H)-yl)methoxy)-4-((1S,5R)-1-methyl- 3,8-diazabicyclo[3.2.1]octan-3-yl)quinazolin-7-yl)-5- ethynylnaphthalen-2-ol, 2TFA | 648 | Column M; Condition: 40% MeOH w/0.1% NH4OH |
| Ex. 144a | 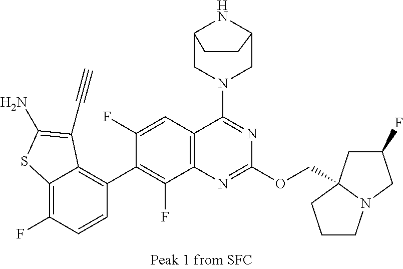 [SMILES: C#Cc1c(N)sc2c(F)ccc(-c3c(F)cc4c(N5CC6CCC(C5)N6)nc(OC[C@@]56CCCN5C[C@H](F)C6)nc4c3F)c12] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2-amino-7-fluorobenzo[b]thio- phene-3-carbonitrile | 624 | Column Daicel chiralcel OJ (250 mm × 30 mm, 10 um) Condition 0.1% NH₃H₂O IPA |
| Ex. 144b | 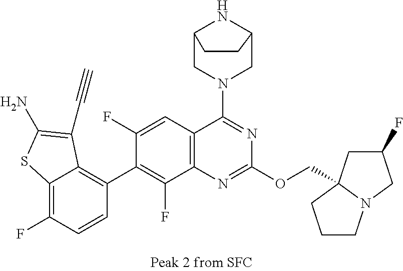 [SMILES: C#Cc1c(N)sc2c(F)ccc(-c3c(F)cc4c(N5CC6CCC(C5)N6)nc(OC[C@@]56CCCN5C[C@H](F)C6)nc4c3F)c12] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2-amino-7-fluorobenzo[b]thio- phene-3-carbonitrile | 624 | Column Daicel chiralcel OJ (250 mm × 30 mm, 10 um) Condition 0.1% NH₃H₂O IPA |
| Ex. 145b | 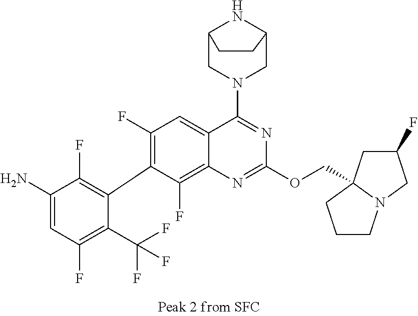 [SMILES: Nc1cc(F)c(C(F)(F)F)c(-c2c(F)cc3c(N4CC5CCC(C4)N5)nc(OC[C@@]45CCCN4C[C@H](F)C5)nc3c2F)c1F] | 3-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2,5-difluoro-4-(trifluoromethyl)- aniline | 629 | Column E; Modifier: 30% MeOH w/0.1% NH4OH |
| Ex. 145a | 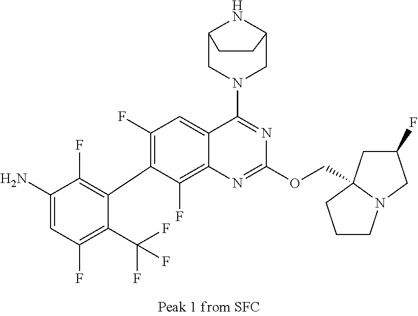 [SMILES: Nc1cc(F)c(C(F)(F)F)c(-c2c(F)cc3c(N4CC5CCC(C4)N5)nc(OC[C@@]45CCCN4C[C@H](F)C5)nc3c2F)c1F] | 3-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2,5-difluoro-4-(trifluoromethyl)- aniline. | 629 | Column E; Modifier: 30% MeOH w/0.1% NH4OH |
| Ex. 146a | 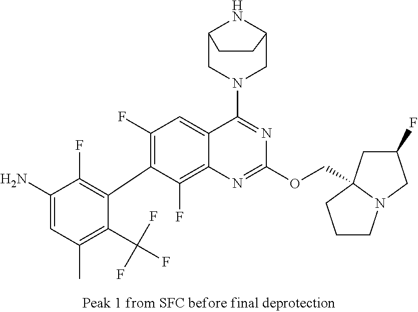 [SMILES: Cc1cc(N)c(F)c(-c2c(F)cc3c(N4CC5CCC(C4)N5)nc(OC[C@@]45CCCN4C[C@H](F)C5)nc3c2F)c1C(F)(F)F] | 3-(4-((1R,5S)-3,8-diazabicyclo[3.2.1]octan-3-yl)-6,8-difluoro- 2-(((2R,7aS)-2-fluorotetrahydro-IH-pyrrolizin-7a(5H)-yl)- methoxy)quinazolin-7-yl)-2-fluoro-5-methyl-4-(trifluoro- methyl)aniline | 625 | Column E; Modifier: 45% MeOH w/0.1% NH4OH |
| Ex. 146b | 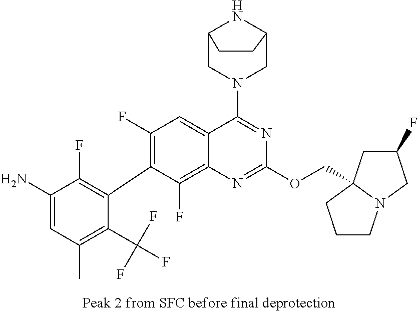 [SMILES: Cc1cc(N)c(F)c(-c2c(F)cc3c(N4CC5CCC(C4)N5)nc(OC[C@@]45CCCN4C[C@H](F)C5)nc3c2F)c1C(F)(F)F] | 3-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2-fluoro-5-methyl-4-(trifluoro- methyl)aniline | 625 | Column E; Modifier: 45% MeOH w/0.1% NH4OH |
| Ex. 147a | 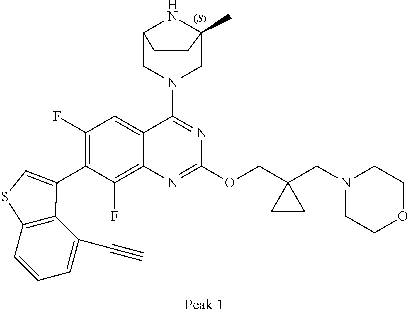 | 4-((1-(((7-(4-ethynylbenzo[b]thiophen-3-yl)-6,8-difluoro-4- ((1S,5R)-1-methyl-3,8-diazabicyclo[3.2.1]octan-3-yl)quin- azolin-2-yl)oxy)methyl)cyclopropyl)methyl)morpholine | 616 | SJ, 21 250 mm, 5um, 25% MeOH w/0.1% NH4OH followed by Lux- 4, 21× 250 mm, 5 um, 35% MeOH w/0.1% NH4OH |
| Ex. 147b | 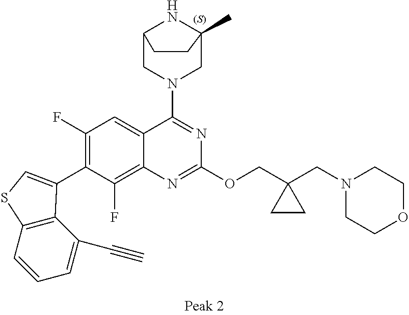 | 4-((1-(((7-(4-ethynylbenzo[b]thiophen-3-yl)-6,8-difluoro-4- ((1S,5R)-1-methyl-3,8-diazabicyclo[3.2.1]octan-3-yl)quin- azolin-2-yl)oxy)methyl)cyclopropyl)methyl)morpholine | 616 | SJ, 21 × 250 mm, 5 um, 25% MeOH w/0.1% NH4OH followed by Lux-4, 21× 250 mm, 5 um, 35% MeOH w/0.1% NH4OH |
| Ex. 148a | 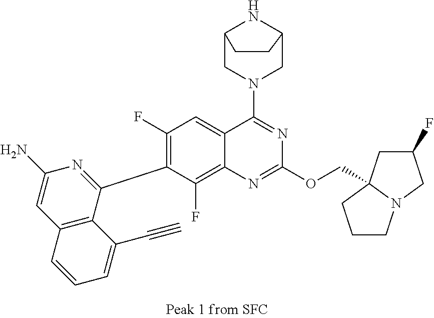 [SMILES: C#Cc1cccc2cc(N)nc(-c3c(F)cc4c(N5CC6CCC(C5)N6)nc(OC[C@@]56CCCN5C[C@H](F)C6)nc4c3F)c12] | 1-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-8-ethynylisoquinolin-3-amine | 600 | Column: CCU, 21 × 250 mm, 5 um Modifier: 50% MeOH w/0.1% NH4OH |
| Ex. 148b | 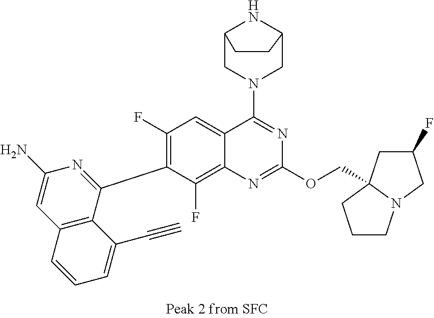 [SMILES: C#Cc1cccc2cc(N)nc(-c3c(F)cc4c(N5CC6CCC(C5)N6)nc(OC[C@@]56CCCN5C[C@H](F)C6)nc4c3F)c12] | 1-(4-((IR,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-8-ethynylisoquinolin-3-amine | 600 | Column: CCU, 21 × 250 mm, 5 um condition: 50% MeOH w/0.1% NH4OH |
| Ex. 149 | 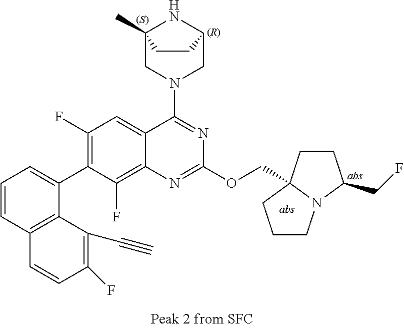 | 7-(8-ethynyl-7-fluoronaphthalen-1-yl)-6,8-difluoro-2- (((38,7aS)-3-(fluoromethyl)tetrahydro-1H-pyrrolizin-7a(5H)- yl)methoxy)-4-((1S,5R)-1-methyl-3,8-diazabicyclo[3.2.1]- octan-3-yl)quinazoline | 630 | |
| Ex. 150 | 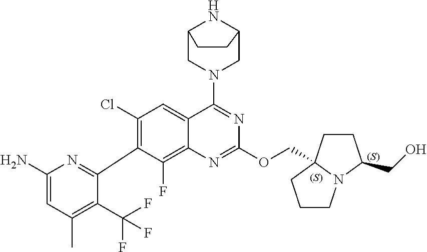 | ((3S,7aS)-7a-(((7-(6-amino-4-methyl-3-trifluoromethyl)- pyridin-2-yl)-4-((1R,5S)-3,8-diazabicyclo[3.2.1]octan-3-yl)- 6-chloro-8-fluoroquinazolin-2-yl)oxy)methyl)hexahydro-1H- pyrrolizin-3-yl)methanol | 636 | NA- |
| Ex. 151a | 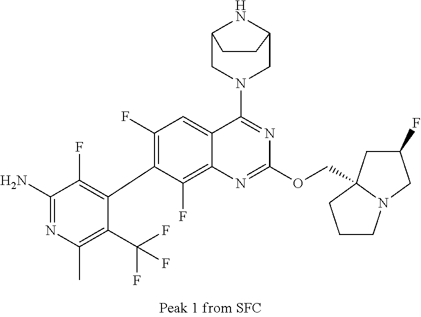 [SMILES: Cc1nc(N)c(F)c(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-3-fluoro-6-methyl-5-(trifluoro- methyl)pyridin-2-amine | 626 | Column B; condition: 35% MeOH w/0.1% NH4OH |
| Ex. 151b | 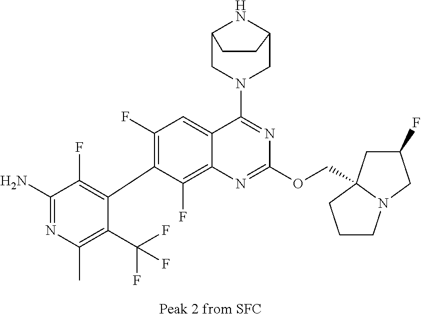 [SMILES: Cc1nc(N)c(F)c(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-3-fluoro-6-methyl-5-(trifluoro- methyl)pyridin-2-amine | 626 | Column E; condition: 35% MeOH w/0.1% NH4OH |
| Ex. 152 | 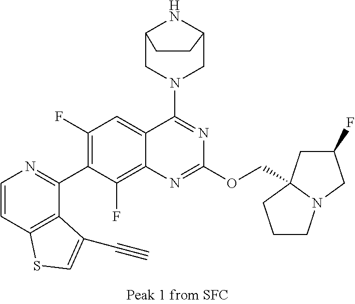 [SMILES: C#Cc1csc2ccnc(-c3c(F)cc4c(N5CC6CCC(C5)N6)nc(OC[C@@]56CCCN5C[C@H](F)C6)nc4c3F)c12] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-3-ethynylthieno[3,2-c]pyridine | 591 | |
| Ex. 154a | 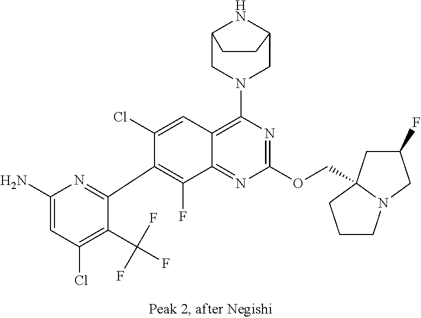 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)n1] | 6-(4-((1R,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-4-chloro-5-(trifluoromethyl)- pyridin-2-amine | 644 | Column: Condition 0.1% NH3H2O ETOH |
| Ex. 154b | 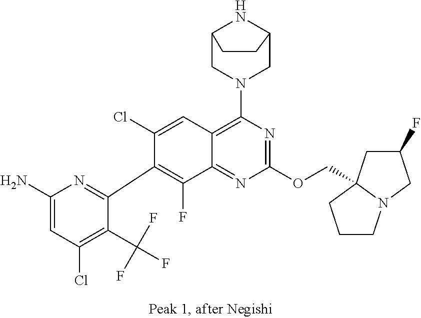 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)n1] | 6-(4-((IR,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-4-chloro-5-(trifluoromethyl)- pyridin-2-amine | 644 | Column: Condition 0.1% NH3H2O ETOH |
| Ex. 155a | 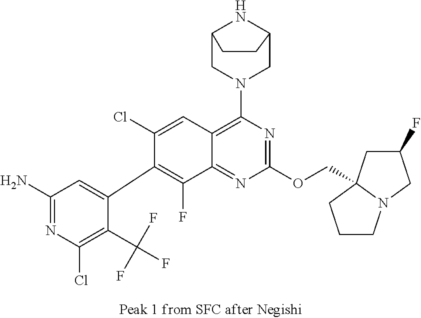 [SMILES: Nc1cc(-c2c(Cl)cc3c(N4CC5CCC(C4)N5)nc(OC[C@@]45CCCN4C[C@H](F)C5)nc3c2F)c(C(F)(F)F)c(Cl)n1] | 4-(4-((1R,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-6-chloro-5-(trifluoromethyl)- pyridin-2-amine | 644 | Prep HPLC Column Welch Xtimate C18 150 × 25 mm × 5 um Condition water (10 mM HCOONH4)-ACN. |
| Ex. 155b | 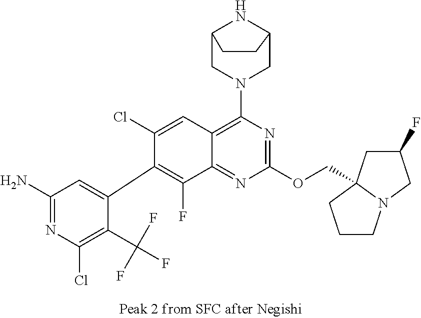 [SMILES: Nc1cc(-c2c(Cl)cc3c(N4CC5CCC(C4)N5)nc(OC[C@@]45CCCN4C[C@H](F)C5)nc3c2F)c(C(F)(F)F)c(Cl)n1] | 4-(4-((1R,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-6-chloro-5-(trifluoromethyl)- pyridin-2-amine | 644 | |
| Ex. 156a | 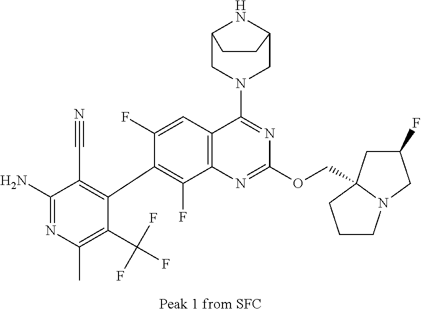 [SMILES: Cc1nc(N)c(C#N)c(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2-amino-6-methyl-5-(trifluoro- methyl)nicotinonitrile | 633 | Column DAICEL CHIRALPAK IC (250 mm × 50 mm, 10 um), Condition: 0.1% NH3H2O IPA |
| Ex. 156b | 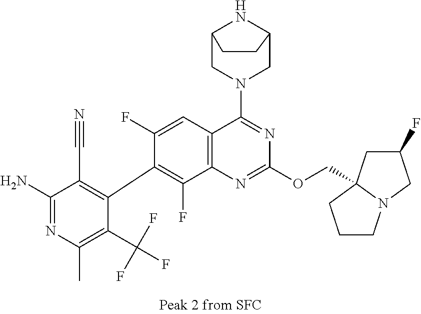 [SMILES: Cc1nc(N)c(C#N)c(-c2c(F)cc3c(N4CC5CCC(C4)N5)nc(OC[C@@]45CCCN4C[C@H](F)C5)nc3c2F)c1C(F)(F)F] | 4-(4-((1R,SS)-3,8-diazabicyclo[3.2.1]octan-3-yl)-6,8-difluoro- 2-(((2R,7aS)-2-fluorotetrahydro-1H-pyrrolizin-7a(5H)-yl)- methoxy)quinazolin-7-yl)-2-amino-6-methyl-5-(trifluoro- methyl)nicotinonitrile | 633 | Column DAICEL CHIRALPAK IC (250 mm × 50 mm, 10 um), Condition: 0.1% NH3H2O IPA |
| Ex. 157a | 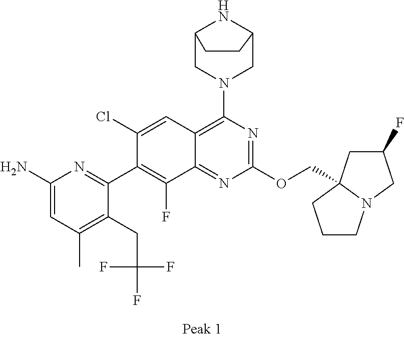 [SMILES: Cc1cc(N)nc(-c2c(Cl)cc3c(N4CC5CCC(C4)N5)nc(OC[C@@]45CCCN4C[C@H](F)C5)nc3c2F)c1CC(F)(F)F] | 6-(4-((1R,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-4-methyl-5-(2,2,2-trifluoroethyl)- pyridin-2-amine | 638 | Column DAICEL CHIRALCEL OJ (250 mm × 30 mm, 10 um) Condition 0.1% NH₃H₂O IPA |
| Ex. 157b | 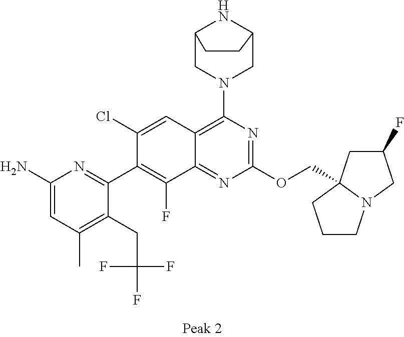 [SMILES: Cc1cc(N)nc(-c2c(Cl)cc3c(N4CC5CCC(C4)N5)nc(OC[C@@]45CCCN4C[C@H](F)C5)nc3c2F)c1CC(F)(F)F] | 6-(4-((1R,5S)-3,8-diazabicyclo[3.2.1]octan-3-yl)-6-chloro-8- fluoro-2-(((2R,7aS)-2-fluorotetrahydro-1H-pyrrolizin-7a(5H)- yl)methoxy)quinazolin-7-yl)-4-methyl-5-(2,2,2-trifluoro- ethyl)pyridin-2-amine | 638 | Column DAICEL CHIRALCEL OJ (250 mm × 30 mm, 10 um) Condition 0.1% NH₃H₂O IPA |

### Example 89: 5-chloro-4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)naphthalen-2-ol (Ex. 89)

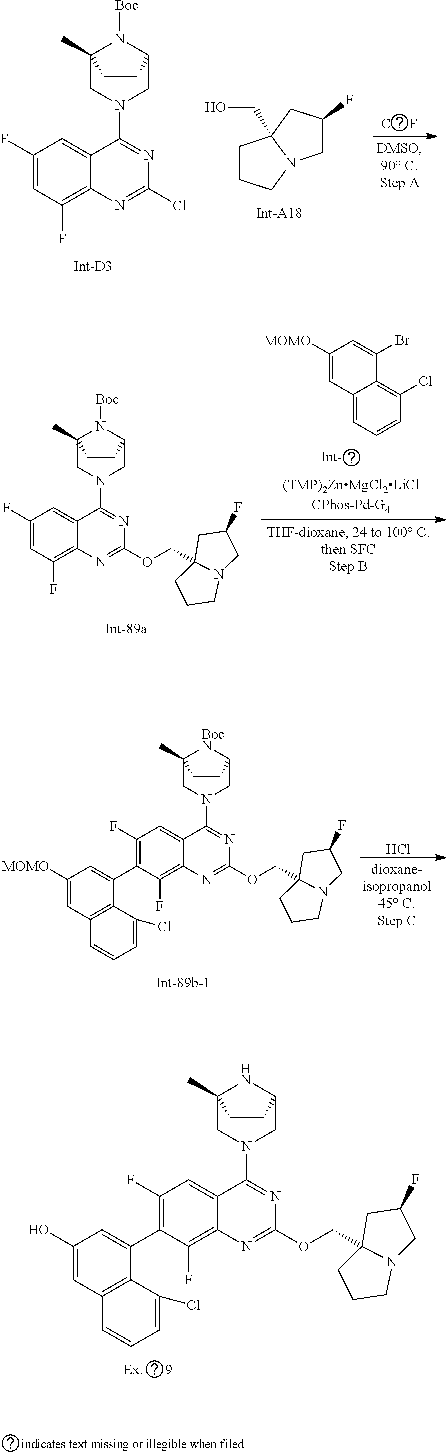

### Step A: tert-butyl (1S,5R)-3-(6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89a)

To a solution of tert-butyl (1S,5R)-3-(2-chloro-6,8-difluoroquinazolin-4-yl)-1-methyl-3,8-diazabicyclo[3.2.1]octane-8-carboxylate (Int-D3, 1.00 g, 2.35 mmol) in DMSO (11 mL) were added ((2R,7aS)-2-fluorotetrahydro-1H-pyrrolizin-7a(5H)-yl)methanol (Int-A1S), 0.450 g, 2.82 mmol) followed by cesium fluoride (1.79 g, 11.8 mmol). The reaction mixture was purged with nitrogen for 5 mins and heated at 90° C. for 16 h. The product mixture was cooled to 24° C. Water was added into the product mixture and solid precipitated was filtered and dried under vacuum. The crude product was purified by column chromatography (Eluent of 35-100% EtOAc/hexanes) to give tert-butyl (1S,5R)-3-(6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89a). MS (ESI): m/z (M+H)⁺ 548.

### Step B: tert-butyl (1S,5R)-3-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)- I-methyl-3,8-diazabicyclo[3.2.1]octane-8-carboxylate (Int-89b-1)

In the glovebox, a 0.15 M solution of TMP₂Zn·MgCl₂LiCl (670 μL, 0.100 mmol) was added in a 30 mL vial. In a separate vial, tert-butyl (1S,5R)-3-(6,8-difluoro-2-(((2R,7aS)-2-fluorotetrahydro- H-pyrrolizin-7a(5H)-yl)methoxy)quinazolin-4-yl)-1-methyl-3,8-diazabicyclo[3.2.1]octane-8-carboxylate (Int-89a, 50 mg, 0.091 mmol) was dissolved in THF (1 mL). The starting material solution was added dropwise into the zinc reagent. The starting material vessel was washed three times with THF (3×300 μL) and the combined rinse was added dropwise into the reaction vessel. The resulting mixture was stirred for 1 h at 24° C. In a third vial, 1-bromo-8-chloro-3-(methoxymethoxy)naphthalene (Int-III4, 55.1 mg, 0.183 mmol) and CPhos-Pd-G4 (11.3 mg, 0.014 mmol) were dissolved in dioxane (1 mL). This solution was added dropwise into the reaction vessel. The vessel containing the electrophile was rinsed with dioxane (3×300 μL). The combined rinses were added dropwise into the reaction vessel. The reaction vessel was removed from the glovebox and then heated at 100° C. for 2h. The product mixture was cooled down to room temperature and diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 100° % ethyl acetate: ethanol-3:1 v/v, linear gradient to afford the racemic product. The material was resolved by SFC (Column E; Conditions: 40% methanol with 0.1% NH₄OH) to afford peak 1 as tert-butyl (1S,5R)-3-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89b-1). MS (ESI), m/z (M+H)⁺ 768. And Peak 2

### Step C: 5-chloro-4-(6,8-difluoro-2-(((2R,7aS)-2-fluorotetrahydro-1H-pyrrolizin-7a(5H)-yl)methoxy)-4-((1S,51?)-1-methyl-3,8-diazabicyclo[3.2.1]octan-3-yl)quinazolin-7-yl)naphthalen-2-ol (Ex. 89)

tert-Butyl (1S,5R)-3-(7-(8-chloro-3-(methoxymethoxy)naphthalen-1-yl)-6,8-difluoro-2-(((2R 7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89b-1, 29 mg, 0.038 mmol) was added in an 8 mL vial. The reaction vessel was evacuated and backfilled with a balloon of nitrogen three times, iso-Propanol (839 μL) was added into the reaction vessel to dissolve the starting material. Then a 4N solution of HCl in dioxane (419 μL) was added dropwise. The resulting mixture was heated at 45° C. for 1 h. The product mixture was concentrated to dryness. The residue obtained was purified via preparatory reverse phase prep-HPLC (MeCN/water with 0.1% TFA modifier) to yield 5-chloro-4-(6,8-difluoro-2-(((2R 7aS)-2-fluorotetrahydro-1H-pyrrolizin-7a(5H)-yl)methoxy)-4-((S, 5R)-1-methyl-3,8-diazabicyclo[3.2.1]octan-3-yl)quinazolin-7-yl)naphthalen-2-ol (Ex. 89). MS (ESI): m/z (M+H)⁺ 624. ¹H NMR (500 MHz, DMSO-d₆) δ 10.81 (s, 1H), 10.41 (s, 1H), 9.28 (s, 2H), 7.88 (dd, J=8.2, 1.4 Hz, 1H), 7.78 (d.. =9.9 Hz, 1H), 7.48-7.38 (m, 3H), 7.10 (d, J=2.5 Hz, 1H), 5.58 (d, J=52,3 Hz, 1H), 4.58 (s, 2H), 4.52-4.34 (m, 2H), 4.21 (s, 1H), 3.95-3.69 (m, 4H), 3.56 (d, J=13.7 Hz. 1H), 2.66-2.51 (m, 1H), 2.39-2.27 (m, 1H), 2.26-2.02 (m, 6H), 2.01-1.88 (m, 1H), 1.84-1.67 (m, 1H), 1.52 (s, 3H).

Examples in the table below were synthesized via an analogous reaction sequence as for Ex. 89 above using intermediates described below.

| | | | | [M + H]⁺ | SFC Purification |
|---|---|---|---|---|---|
| Ex. | Starting Material | Structure | Compound Name | Found | Conditions |
| Ex. 158 | 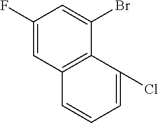 [SMILES: Fc1cc(Br)c2c(Cl)cccc2c1] | 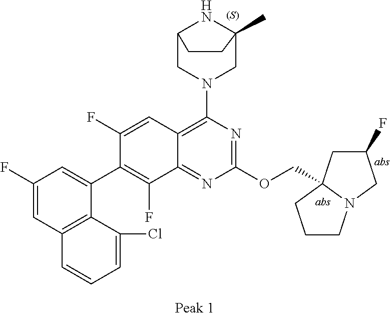 | 7-(8-chloro-3-fluoro- naphthalen-1-yl)-6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazoline | 626 | Column: YMC-Actus Triart C18 100 × 30 mm × 5 um; Condition: water(0.1%TFA)-ACN |
| Ex. 159 | 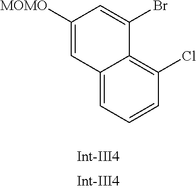 | 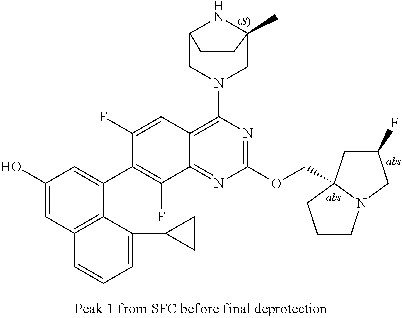 | 5-cyclopropyl-4-(6,8- difluoro-2-(((2R)-2- fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- naphthalen-2-ol | 630 | Column C; Condition 0.1% NH3H2O ETOH |
| Ex. 160a | 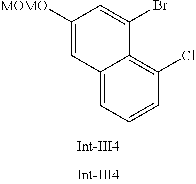 | 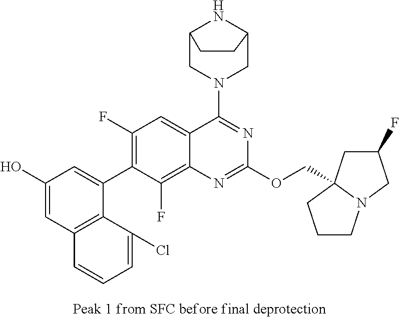 [SMILES: Oc1cc(-c2c(F)cc3c(N4CC5CCC(C4)N5)nc(OC[C@@]45CCCN4C[C@H](F)C5)nc3c2F)c2c(Cl)cccc2c1] | 4-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-yl)- methoxy)quinazolin- 7-yl)-5-chloronaph- thalen-2-ol | 610 | Column M; Modifier: 40% IPA w/0.1% NH4OH |
| Ex. 160b | 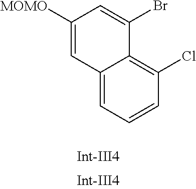 | 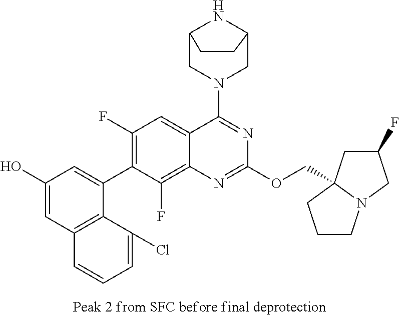 [SMILES: Oc1cc(-c2c(F)cc3c(N4CC5CCC(C4)N5)nc(OC[C@@]45CCCN4C[C@H](F)C5)nc3c2F)c2c(Cl)cccc2c1] | 4-(4-((1R,5S)-3,8- diazabicyclo[3.2.1]- octan-3-yl)-6,8- difluoro-2-(((2R,7aS)- 2-fluorotetrahydro- 1H-pyrrolizin-7a(5H)- yl)methoxy)quinazolin- 7-yl)-5-chloronaph- thalen-2-ol | 610 | Column M; Modifier: 40% IPA w/0.1% NH4OH |
| Ex. 161 | 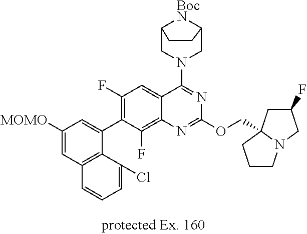 | 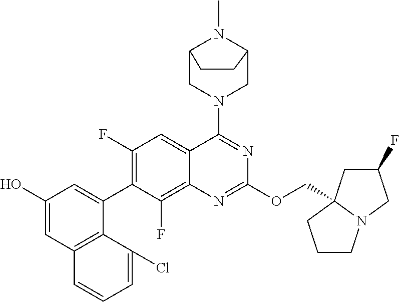 [SMILES: CN1C2CCC1CN(c1nc(OC[C@@]34CCCN3C[C@H](F)C4)nc3c(F)c(-c4cc(O)cc5cccc(Cl)c45)c(F)cc13)C2] | 5-chloro-4-(6,8- difluoro-2-(((2R,7aS)- 2-fluorotetrahydro-1H- pyrrolizin-7a(5H)-yl)- methoxy)-4-((1R,5S)- 8-methyl-3,8-diaza- bicyclo[3.2.1]octan-3- yl)quinazolin-7-yl)- naphthalen-2-ol | 624 | |
| Ex. 162a | 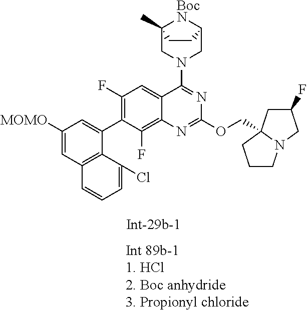 | 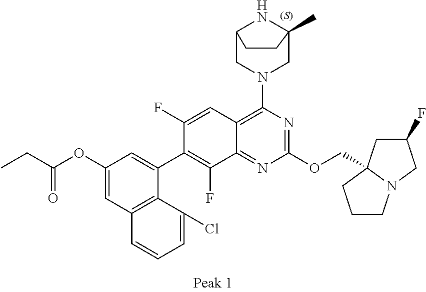 | 5-chloro-4-(6,8- difluoro-2-(((2R)-2- fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- naphthalen-2-yl pro- pionate | 680 | |
| Ex. 162b | 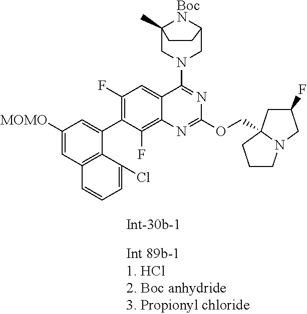 | 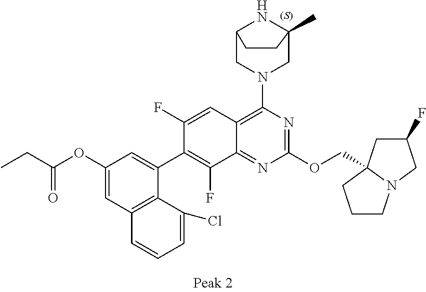 | 5-chloro-4-(6,8- difluoro-2-(((2R)-2- fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- naphthalen-2-yl pro- pionate | 680 | |
| Ex. 163a | 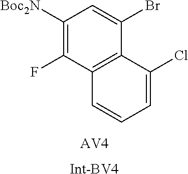 | 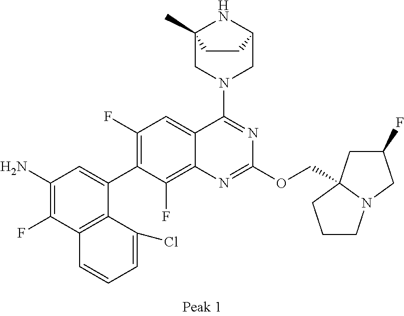 [SMILES: C[C@]12CC[C@H](CN(c3nc(OC[C@@]45CCCN4C[C@H](F)C5)nc4c(F)c(-c5cc(N)c(F)c6cccc(Cl)c56)c(F)cc34)C1)N2] | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-1- fluoronaphthalen-2- amine, 2TFA | 641 | Column E; Condition: 40% MeOH w/0.1% NH4OH |
| Ex. 163b | 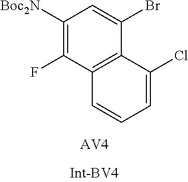 | 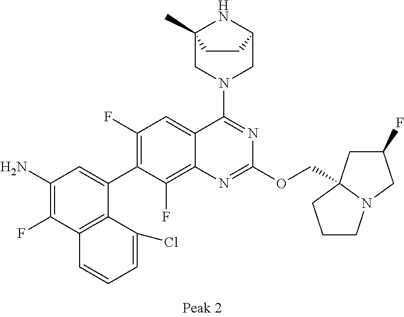 [SMILES: C[C@]12CC[C@H](CN(c3nc(OC[C@@]45CCCN4C[C@H](F)C5)nc4c(F)c(-c5cc(N)c(F)c6cccc(Cl)c56)c(F)cc34)C1)N2] | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-1- fluoronaphthalen-2- amine, 2TFA | 641 | Column E; 40% MeOH w/0.1% NH4OH |
| Ex. 164 | 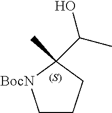 | 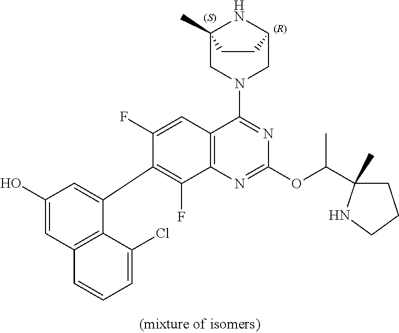 | 5-chloro-4-(6,8- difluoro-4-((1S,5R)-1- methyl-3,8-diaza- bicyclo-[3.2.1]octan-3- yl)-2-(1-((S)-2-methyl- pyrrolidin-2-yl)ethoxy)- quinazolin-7-yl)naph- thalen-2-ol, TFA | 594.2 | |
| Ex. 165 | 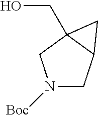 [SMILES: CC(C)(C)OC(=O)N1CC2CC2(CO)C1] | 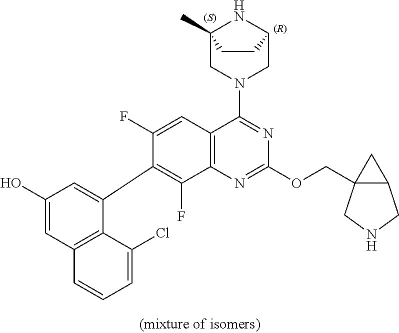 | 4-(2-((3-azabicyclo- [3.1.0]hexan-1-yl)- methoxy)-6,8-difluoro- 4-((1S,5R)-1-methyl- 3,8-diazabicyclo[3.2.1]- octan-3-yl)quinazolin- 7-yl)-5-chloronaph- thalen-2-ol, TFA | 578.2 | -NA- |
| Ex. 166a | 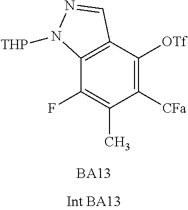 | 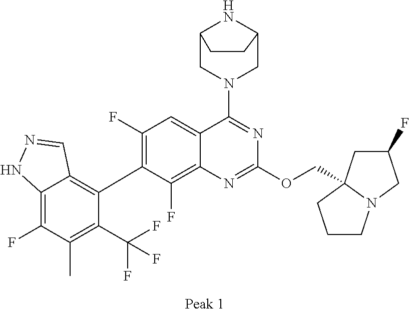 [SMILES: Cc1c(C(F)(F)F)c(-c2c(F)cc3c(N4CC5CCC(C4)N5)nc(OC[C@@]45CCCN4C[C@H](F)C5)nc3c2F)c2cn[nH]c2c1F] | 4-((1R,5S)-3,8-diaza- bicyclo[3.2.1]octan-3- yl)-6,8-difluoro-7-(7- fluoro-6-methyl-5- (trifluoromethyl)-1H- indazol-4-yl)-2- (((2R,7aS)-2-fluoro- tetrahydro-1H-pyrrol- izin-7a(5H)-yl)meth- oxy)quinazoline, 2TFA | 650 | Column N: 10% MeOH w/0.1% NH4OH |
| Ex. 166b | 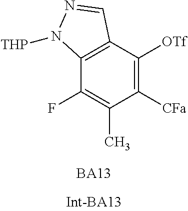 | 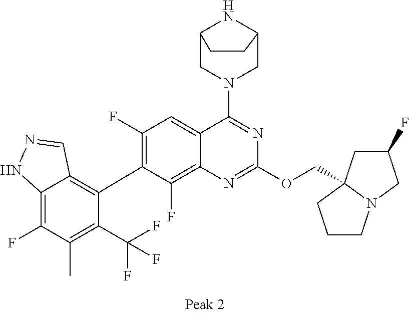 [SMILES: Cc1c(C(F)(F)F)c(-c2c(F)cc3c(N4CC5CCC(C4)N5)nc(OC[C@@]45CCCN4C[C@H](F)C5)nc3c2F)c2cn[nH]c2c1F] | 4-((1R,5S)-3,8-diaza- bicyclo[3.2.1]octan-3- yl)-6,8-difluoro-7-(7- fluoro-6-methyl-5- (trifluoromethyl)-1H- indazol-4-yl)-2- (((2R,7aS)-2-fluoro- tetrahydro-1H-pyrrol- izin-7a(5H)-yl)meth- oxy)quinazoline, 2TFA | 650 | Column N: 10% MeOH w/0.1% NH4OH |
| Ex. 167 | 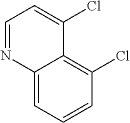 [SMILES: Clc1cccc2nccc(Cl)c12] | 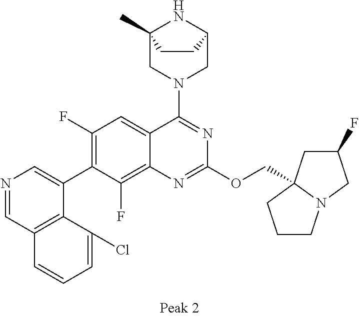 [SMILES: C[C@]12CC[C@H](CN(c3nc(OC[C@@]45CCCN4C[C@H](F)C5)nc4c(F)c(-c5cncc6cccc(Cl)c56)c(F)cc34)C1)N2] | 7-(5-chloroisoquinolin- 4-yl)-6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo-[3.2.1]octan-3- yl)quinazoline, 2TFA | 609 | Column Q Condition: 30% IPA w/0.1% NH4OH |
| Ex. 168 | 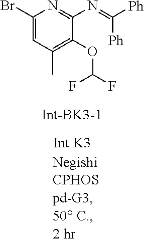 [SMILES: Cc1cc(Br)nc(N=C(c2ccccc2)c2ccccc2)c1OC(F)F] | 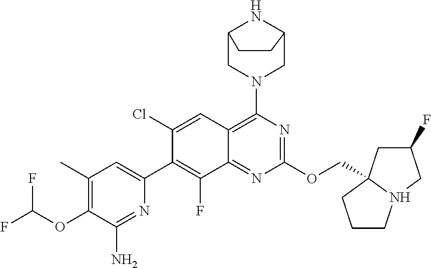 [SMILES: Cc1cc(-c2c(Cl)cc3c(N4CC5CCC(C4)N5)nc(OC[C@@]45CCCN4C[C@H](F)C5)nc3c2F)nc(N)c1OC(F)F] | 6-(4-((1R,5S)-3,8- diazabicyclo[3.2.1]- octan-3-yl)-6-chloro-8- fluoro-2-(((2R,7aS)-2- fluorotetrahydro-1H- pyrrolizin-7a(5H)-yl)- methoxy)quinazolin-7- yl)-3-(difluorometh- oxy)-4-methylpyridin- 2-amine | 622 | -NA- |
| Ex. 169 | 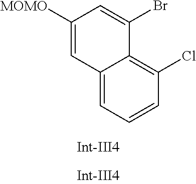 | 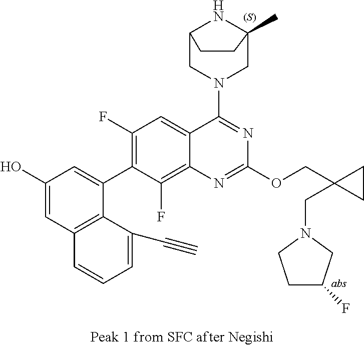 | 4-(6,8-difluoro-2-((1- (((R)-3-fluoropyrrol- idin-1-yl)methyl)cyclo- propyl)methoxy)-4- ((1S,5R)-1-methyl-3,8- diazabicyclo[3.2.1]- octan-3-yl)quinazolin- 7-yl)-5-ethynylnaph- thalen-2-ol | 628 | Column R; Modifier: 15% IPA w/0.1% NH4OH |
| Ex. 170b | Int BN7 | 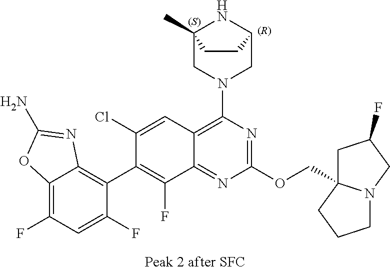 | 4-(6-chloro-8-fluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)- 5,7-difluorobenzo[d]- oxazol-2-amine. | 632 | SFC final step: Column: DAICEL CHIRALCEL OD-H (250 mm × 30 mm, 5 um); Condition: 0.1% NH₃H₂O EtOH |
| Ex. 170a | Int BN7 | 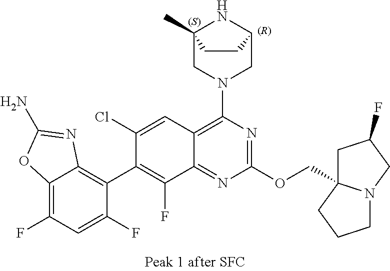 | 4-(6-chloro-8-fluoro-2- (((2R,7aS)-2-fluoro- tetrahydro-1H-pyrrol- izin-7a(5H)-yl)meth- oxy)-4-((1S,5R)-1- methyl-3,8-diaza- bicyclo-[3.2.1]octan- 3-yl)quinazolin-7-yl)- 5,7-difluorobenzo[d]- oxazol-2-amine | 632 | SFC final step: Column: DAICEL CHIRALCEL OD-H (250 mm × 30 mm, 5 um); Condition: 0.1% NH₃H₂O EtOH |
| Ex. 171b | 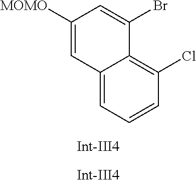 | 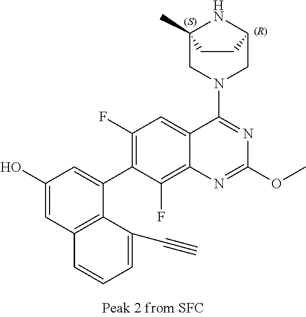 | 4-(6,8-difluoro-2- methoxy-4-((1S,5R)-1- methyl-3,8-diaza- bicyclo-[3.2.1]octan-3- yl)quinazolin-7-yl)-5- ethynylnaphthalen-2- ol, TFA | 487 | Column: IC, 21 × 250 mm, 5 um Modifier: 40% IPA w/0.1% NH4OH |
| Ex. 171a | 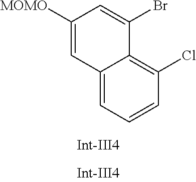 | 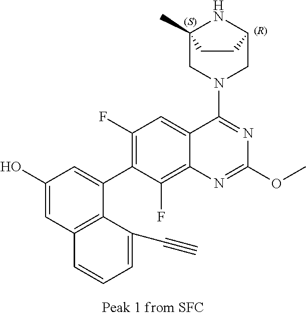 | 4-(6,8-difluoro-2-meth- oxy-4-((18,5R)-1-meth- yl-3,8-diazabicyclo- [3.2.1]octan-3-yl)quin- azolin-7-yl)-5-ethynyl- naphthalen-2-ol, TFA | 487 | Column: IC, 21 × 250 mm, 5 um Modifier: 40% IPA w/0.1% NH4OH |
| Ex. 172 | 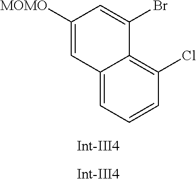 | 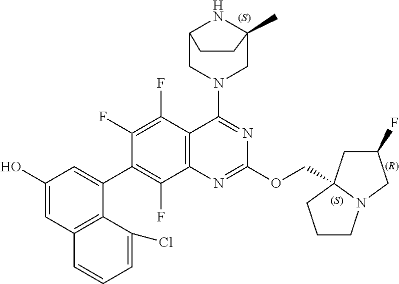 | 5-chloro-4-(5,6,8- trifluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- naphthalen-2-ol, 2TFA | 642 | |
| Ex. 173-1a | 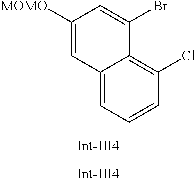 | 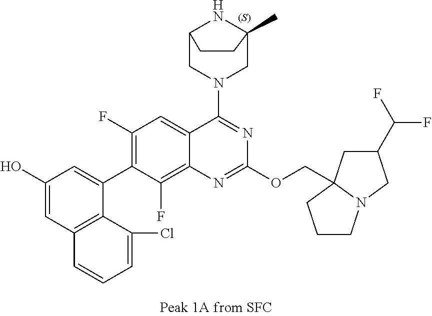 | 5-chloro-4-(2-((2- (difluoromethyl)tetra- hydro-1H-pyrrolizin- 7a(5H)-yl)methoxy)- 6,8-difluoro-4- ((1S,5R)-1-methyl- 3,8-diazabicyclo- [3.2.1]octan-3-yl)- quinazolin-7-yl)naph- thalen-2-ol | 656 | SFC before depro- tection, Column D; Condition: 0.1% NH₃H₂O MeOH |
| Ex. 173-1b | 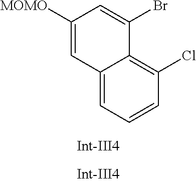 | 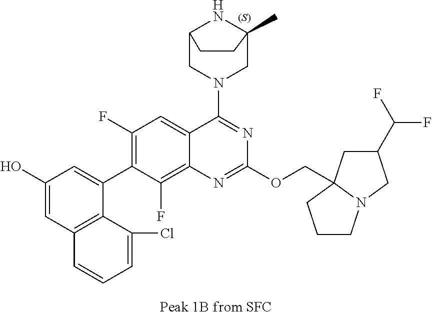 | 5-chloro-4-(2-((2- (difluoromethyl)tetra- hydro-1H-pyrrolizin- 7a(5H)-yl)methoxy)- 6,8-difluoro-4-((1S,5R)- 1-methyl-3,8-diaza- bicyclo[3.2.1]octan-3- yl)quinazolin-7-yl)- naphthalen-2-ol | 656 | SFC before depro- tection,Column D; Condition: 0.1% NH₃H₂O MeOH |
| Ex. 173-2a | 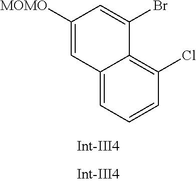 | 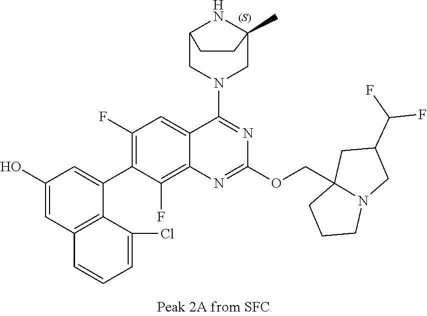 | 5-chloro-4-(2-((2- (difluoromethyl)tetra- hydro-1H-pyrrolizin- 7a(5H)-yl)methoxy)- 6,8-difluoro-4- ((1S,5R)-1-methyl- 3,8-diazabicyclo- [3.2.1]octan-3-yl)- quinazolin-7-yl)naph- thalen-2-ol | 656 | SFC before depro- tection Column D; Condition: 0.1% NH₃H₂O MeOH |
| Ex. 173-2b | 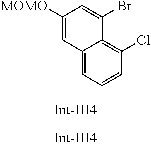 | 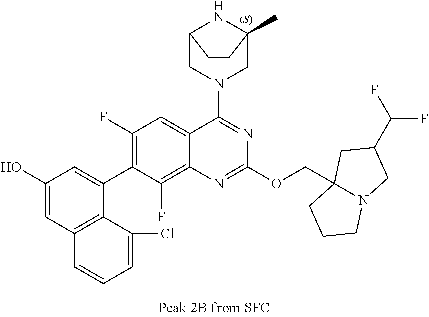 | 5-chloro-4-(2-((2- (difluoromethyl)tetra- hydro-1H-pyrrolizin- 7a(5H)-yl)methoxy)- 6,8-difluoro-4- ((1S,5R)-1-methyl- 3,8-diazabicyclo[3.2.1]- octan-3-yl)quinazolin- 7-yl)naphthalen-2-ol | 656 | SFC before depro- tection Column D; Condition: 0.1% NH₃H₂O MeOH |
| Ex. 174a | 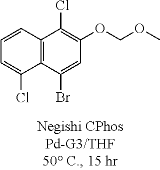 [SMILES: COCOc1cc(Br)c2c(Cl)cccc2c1Cl] | 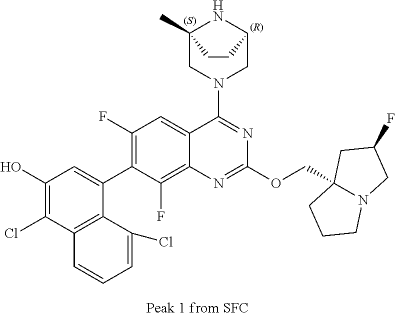 | 1,5-dichloro-4-(6,8- difluoro-2-(((2R,7aS)- 2-fluorotetrahydro-1H- pyrrolizin-7a(5H)-yl)- methoxy)-4-((18,5R)- 1-methyl-3,8-diaza- bicyclo[3.2.1]octan-3- yl)quinazolin-7-yl)- naphthalen-2-ol. | 657 | SFC before final deprotection, Column A; Condition: 0.1% NH₃H₂O EtOH |
| Ex. 174b | 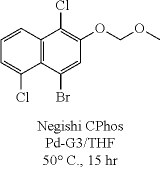 [SMILES: COCOc1cc(Br)c2c(Cl)cccc2c1Cl] | 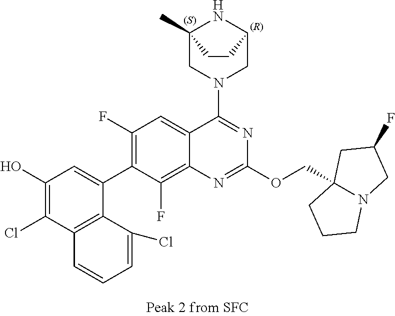 | 1,5-dichloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- naphthalen-2-ol. | 657 | SFC before final deprotection, Column A; Condition: 0.1% NH₃H₂O EtOH |
| Ex. 175a | 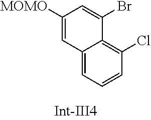 | 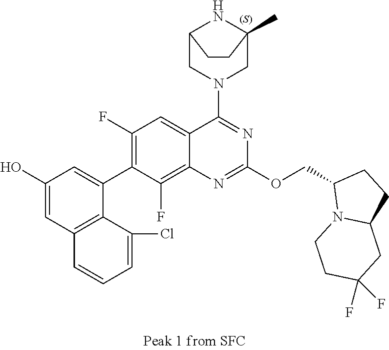 | 5-chloro-4-(2-(((38, 8aS)-7,7-difluoroocta- hydroindolizin-3-yl)- methoxy)-6,8-difluoro- 4-((1S,5R)-1-methyl- 3,8-diazabicyclo[3.2.1]- octan-3-yl)quinazolin- 7-yl)naphthalen-2-ol | 656 | SFC separation before final deprotection, Column A; Condition 0.1% NH₃H₂O IPA |
| Ex. 175b | 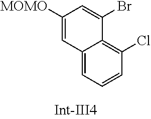 | 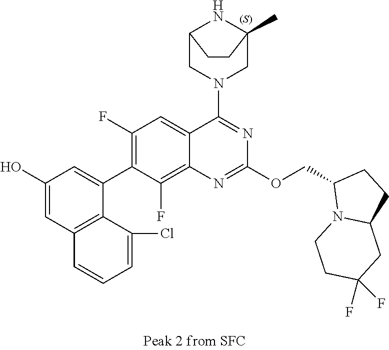 | 5-chloro-4-(2-(((35, 8aS)-7,7-difluoroocta- hydroindolizin-3-yl)- methoxy)-6,8-difluoro- 4-((1S,5R)-1-methyl- 3,8-diazabicyclo[3.2.1]- octan-3-yl)quinazolin- 7-yl)naphthalen-2-ol | 656 | SFC separation before final deprotection, Column A; Condition 0.1% NH₃H₂O IPA |
| Ex. 176a | 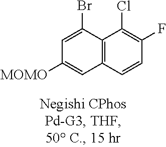 | 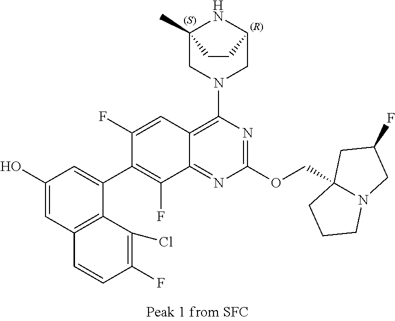 | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-6- fluoronaphthalen-2-ol. | 642 | SFC before final deprotection Column A; Condition 0.1% NH₃H₂O IPA |
| Ex. 176b | 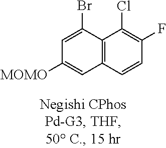 | 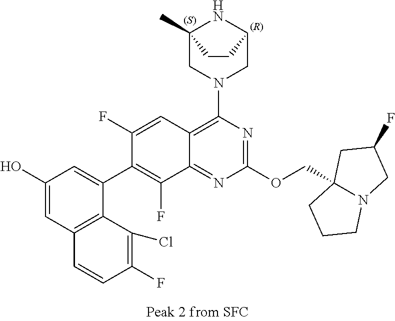 | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-6- fluoronaphthalen-2-ol. | 642 | SFC before final deprotection Column A; Condition 0.1% NH₃H₂O IPA |
| Ex. 177a | 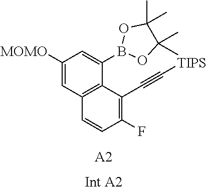 | 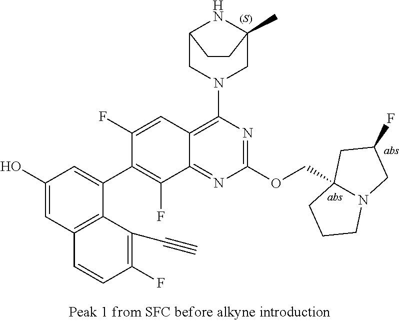 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethynyl-6-fluoronaph- thalen-2-ol | 632 | SFC before alkyne introduction Column A; Condition 0.1% NH₃H₂O IPA |
| Ex. 177b | 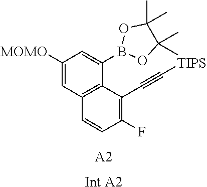 | 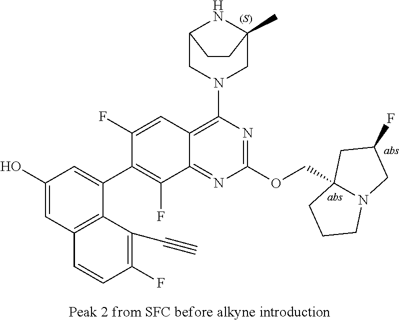 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethynyl-6-fluoronaph- thalen-2-ol | 632 | SFC before alkyne introduction Column A; Condition 0.1% NH₃H₂O IPA |
| Ex. 178a | Using Int Ex. 89 | 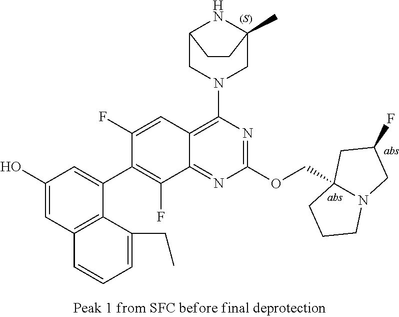 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethylnaphthalen-2-ol | 618 | Column A; Condition; 0.1% NH3H2O IPA |
| Ex. 178b | Using Int Ex. 89 | 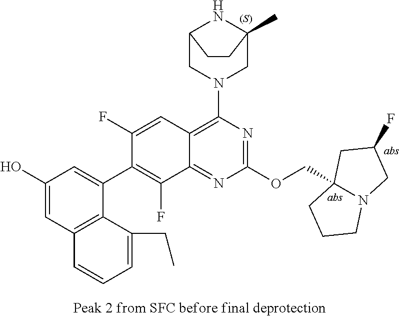 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethylnaphthalen-2-ol | 618 | Column A; Condition: 0.1% NH3H2O IPA |
| Ex. 179a | Using Ex. 89 | 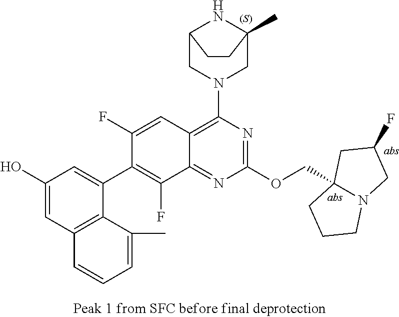 | 4-(6,8-difluoro-2- (((2R)-2-fluorotetra- hydro-1H-pyrrolizin- 7a(5H)-yl)methoxy)-4- ((1S,5R)-1-methyl-3,8- diazabicyclo[3.2.1]- octan-3-yl)quinazolin- 7-yl)-5-methylnaph- thalen-2-ol | 604 | Column C; Condition: 0.1% NH3H2O ETOH |
| Ex. 179b | Using Ex. 89 | 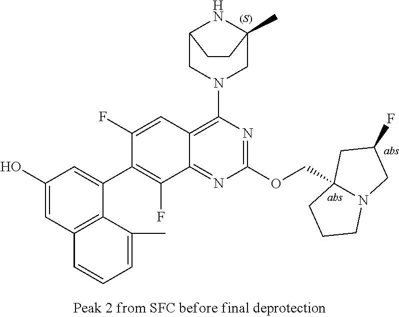 | 4-(6,8-difluoro-2- (((2R)-2-fluorotetra- hydro-1H-pyrrolizin- 7a(5H)-yl)methoxy)-4- ((1S,5R)-1-methyl-3,8- diazabicyclo[3.2.1]- octan-3-yl)quinazolin- 7-yl)-5-methylnaph- thalen-2-ol | 604 | Column C; Condition: 0.1% NH3H2O ETOH |
| Ex. 180a | 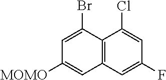 | 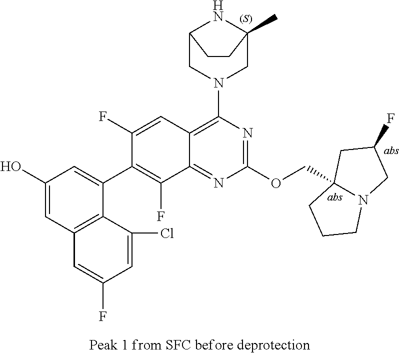 | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo-[3.2.1]octan-3- yl)quinazolin-7-yl)-7- fluoronaphthalen-2-ol | 642 | Column A; Condition: 0.1% NH3H2O ETOH |
| Ex. 180b | 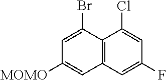 | 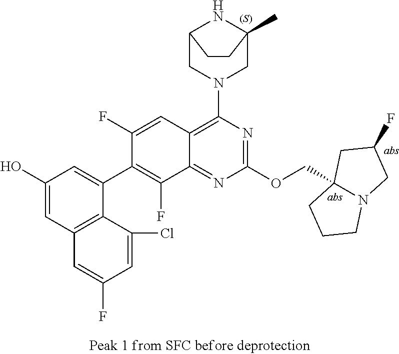 | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-7- fluoronaphthalen-2-ol | 642 | Column A; Condition; 0.1% NH3H2O ETOH |
| Ex. 181a | Int 181C | 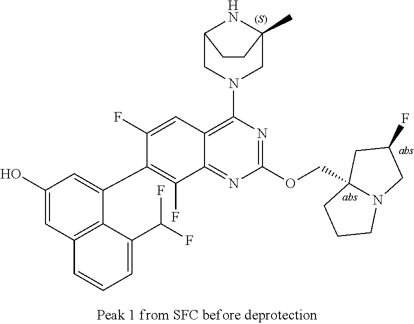 | rac-4-(6,8-difluoro-2- (((2R,7aS)-2-fluoro- tetrahydro-1H-pyrrol- izin-7a(5H)-yl)meth- oxy)-4-((18,5R)-1- methyl-3,8-diaza- bicyclo[3.2.1]octan-3- yl)quinazolin-7-yl)-5- (difluoromethyl)naph- thalen-2-ol | 640 | Column K; condition: 0.1% NH3H2O IPA; |
| Ex. 181b | Int 181C | 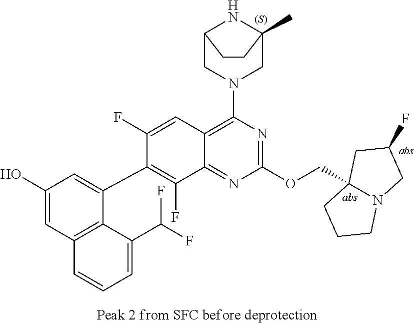 | rac-4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- (difluoromethyl)naph- thalen-2-ol | 640 | Column K; condition: 0.1% NH3H2O IPA |
| Ex. 182 | | 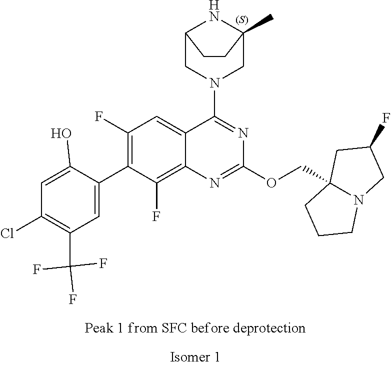 | 3-chloro-5-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-4- (trifluoromethyl)phenol | 642 | Column: Chiralcel OJ- 3150; Á4.6 mm I.D., 3 um condition: A: CO2 B: ethanol (0.05% DEA) |
| Ex. 256 | | 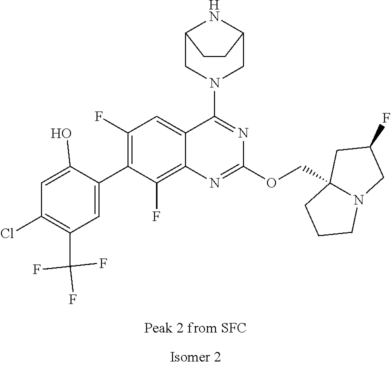 [SMILES: Oc1cc(Cl)c(C(F)(F)F)cc1-c1c(F)cc2c(N3CC4CCC(C3)N4)nc(OC[C@@]34CCCN3C[C@H](F)C4)nc2c1F] | 2-(4-((1R,5S)-3,8- diazabicyclo[3.2.1]- octan-3-yl)-6,8- difluoro-2-(((2R,7aS)- 2-fluoro-tetrahydro- 1H-pyrrolizin-7a(5H)- yl)methoxy)quin- azolin-7-yl)-5-chloro- 4-(trifluoromethyl)- phenol | 628 | |
| Ex. 183a | | 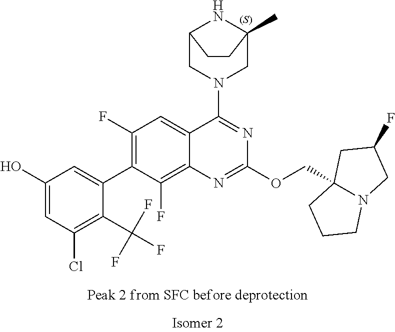 | 3-chloro-5-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)-4- (trifluoromethyl)phenol | 642 | Column: Chiralcel OJ- 3150; Á4.6 mm I.D., 3 um condition: A: CO2 B: ethanol (0.05% DEA) |
| Ex. 183b | | 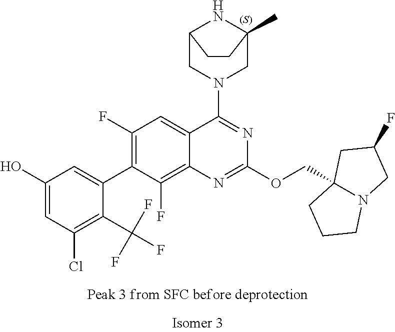 | 3-chloro-5-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo-[3.2.1]octan-3- yl)quinazolin-7-yl)-4- (trifluoromethyl)phenol | 642 | Column: Chiralcel OJ- 3150; Á4.6 mm I.D., 3 um condition: A: CO2 B: ethanol (0.05% DEA) |
| Ex. 184 | 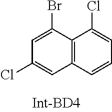 [SMILES: Clc1cc(Br)c2c(Cl)cccc2c1] | 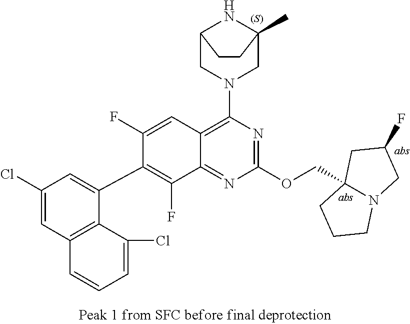 | 7-(3,7-dichloronaph- thalen-1-yl)-6,8- difluoro-2-(((2R,7aS)- 2-fluorotetrahydro- 1H-pyrrolizin-7a(5H)- yl)methoxy)-4-((1S, 5R)-1-methyl-3,8 diazabicyclo[3.2.1]- octan-3-yl)quinazoline | 642 | Column K; Condition 0.1% NH3H2O ETOH |
| Ex. 185 | 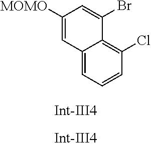 | 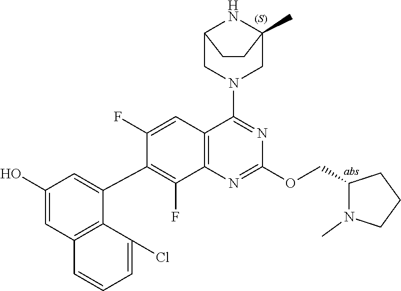 | 5-chloro-4-(6,8- difluoro-4-((1S,5R)- 1-methyl-3,8-diaza- bicyclo-[3.2.1]octan- 3-yl)-2-(((S)-1-methyl- pyrrolidin-2-yl)meth- oxy)quinazolin-7-yl)- naphthalen-2-ol | 580 | |
| Ex. 186a | 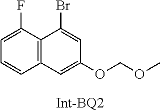 [SMILES: COCOc1cc(Br)c2c(F)cccc2c1] | 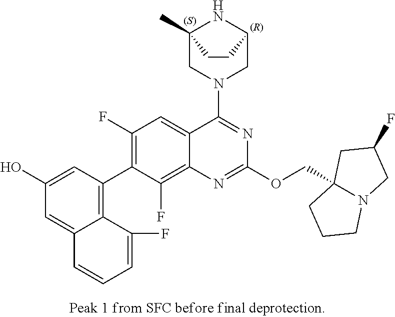 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- fluoronaphthalen-2-ol | 608 | Column: Chiralpak IC- 3100; Á4.6 mm I.D., 3 um Condition A: CO2 B:ethanol (0.05% DEA) |
| Ex. 186b | 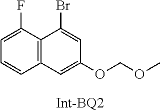 [SMILES: COCOc1cc(Br)c2c(F)cccc2c1] | 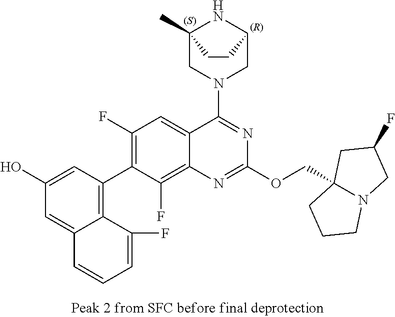 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- fluoronaphthalen-2-ol | 608 | Column: Chiralpak IC- 3100; Á4.6 mm I.D., 3 um Condition A: CO2 B: ethanol (0.05% DEA) |
| Ex. 187 | 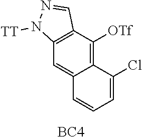 | 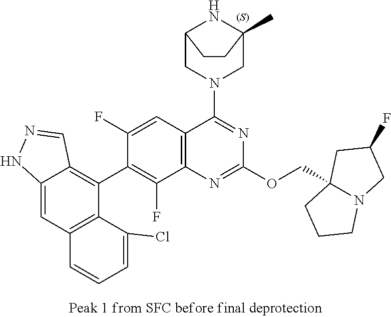 | 5-chloro-4-(6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazolin-7-yl)- 1H-benzo[f]indazole | 648 | Column C; Condition 0.1% NH₃H₂O IPA |
| Ex. 188 | 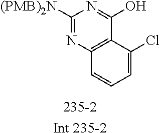 | 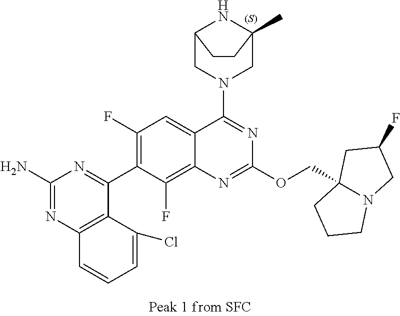 | 5-chloro-6′,8′-difluoro- 2′-(((2R,7aS)-2-fluoro- tetrahydro-1H-pyrrol- izin-7a(5H)-yl)meth- oxy)-4'-((1S,5R)-1- methyl-3,8-diaza- bicyclo[3.2.1]octan-3- yl)-[4,7′-biquinazolin]- 2-amine | 625 | Column L; Modifier: 40% IPA w/0.1% NH4OH |
| Ex. 189a | 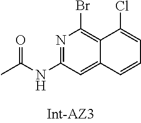 [SMILES: CC(=O)Nc1cc2cccc(Cl)c2c(Br)n1] | 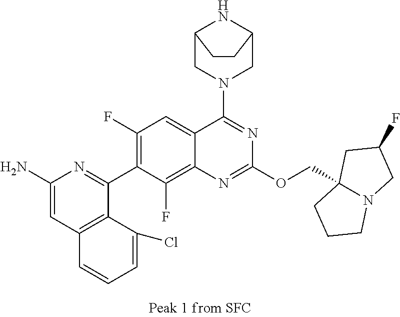 [SMILES: Nc1cc2cccc(Cl)c2c(-c2c(F)cc3c(N4CC5CCC(C4)N5)nc(OC[C@@]45CCCN4C[C@H](F)C5)nc3c2F)n1] | 1-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-yl)- methoxy)quinazolin- 7-yl)-8-chloroisoquin- olin-3-amine | 610 | Column L; Condition: 45% MeOH w/0.1% NH4OH |
| Ex. 189b | 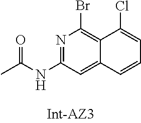 [SMILES: CC(=O)Nc1cc2cccc(Cl)c2c(Br)n1] | 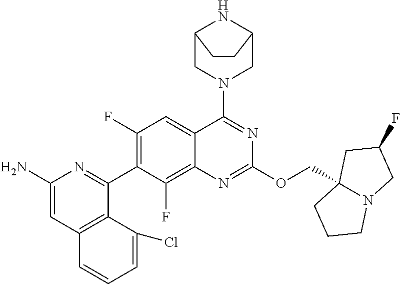 [SMILES: Nc1cc2cccc(Cl)c2c(-c2c(F)cc3c(N4CC5CCC(C4)N5)nc(OC[C@@]45CCCN4C[C@H](F)C5)nc3c2F)n1] | 1-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-yl)- methoxy)quinazolin-7- yl)-8-chloroisoquin- olin-3-amine | 610 | Column L; Condition: 45% MeOH w/0.1% NH4OH |
| Ex. 190b | 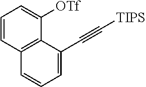 | 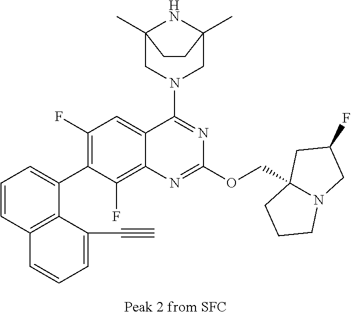 [SMILES: C#Cc1cccc2cccc(-c3c(F)cc4c(N5CC6(C)CCC(C)(C5)N6)nc(OC[C@@]56CCCN5C[C@H](F)C6)nc4c3F)c12] | 4-(1,5-dimethyl-3,8- diazabicyclo[3.2.1]- octan-3-yl)-7-(8- ethynylnaphthalen-1- yl)-6,8-difluoro-2- (((2R,7aS)-2-fluoro- tetrahydro-1H-pyrrol- izin-7a(5H)-yl)meth- oxy)quinazoline, TFA | 612 | Column AS-H, 21 × 250 mm, 5 um Condition: 25% MeOH w/0.1% NH4OH |
| Ex. 190a | 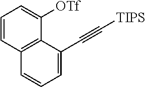 | 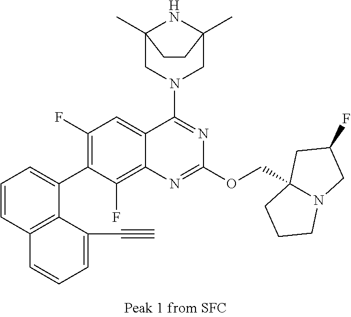 [SMILES: C#Cc1cccc2cccc(-c3c(F)cc4c(N5CC6(C)CCC(C)(C5)N6)nc(OC[C@@]56CCCN5C[C@H](F)C6)nc4c3F)c12] | 4-(1,5-dimethyl-3,8- diazabicyclo[3.2.1]- octan-3-yl)-7-(8- ethynylnaphthalen-1- yl)-6,8-difluoro-2- (((2R,7aS)-2-fluoro- tetrahydro-1H-pyrrol- izin-7a(5H)-yl)meth- oxy)-quinazoline, TFA | 612 | Column AS-H, 21 × 250 mm, 5 um Condition: 25% MeOH w/0.1% NH4OH |
| Ex. 191a | 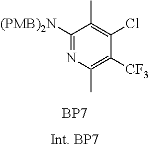 | 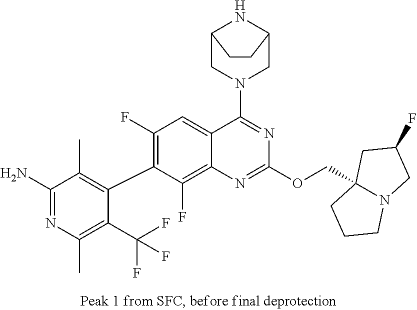 [SMILES: Cc1nc(N)c(C)c(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-l)- methoxy)quinazolin-7- yl)-3,6-dimethyl-5- (trifluoromethyl)pyr- idin-2-amine | 622 | Column E; Condition: 40% IPA w/0.1% NH4OH |
| Ex. 191b | 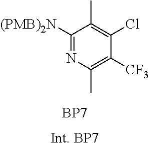 | 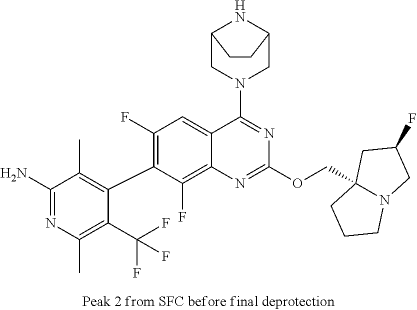 [SMILES: Cc1nc(N)c(C)c(-c2c(F)cc3c(N4CC5CCC(C4)N5)nc(OC[C@@]45CCCN4C[C@H](F)C5)nc3c2F)c1C(F)(F)F] | 4-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-l)- methoxy)quinazolin-7- yl)-3,6-dimethyl-5- (trifluoromethyl)pyr- idin-2-amine | 622 | Column E; Condition: 40% IPA w/0.1% NH4OH |
| Ex. 192a | 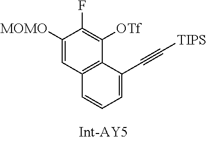 | 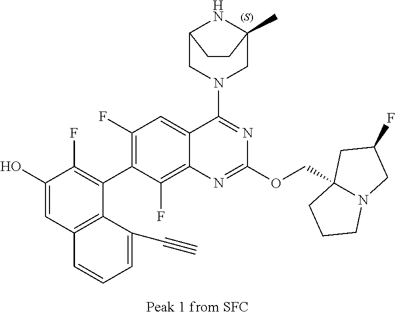 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethynyl-3-fluoronaph- thalen-2-ol, 2TFA | 632 | Column B; Condition: 20% IPA w/0.1% NH4OH |
| Ex. 192b | 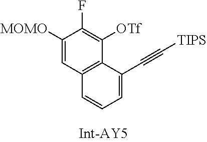 | 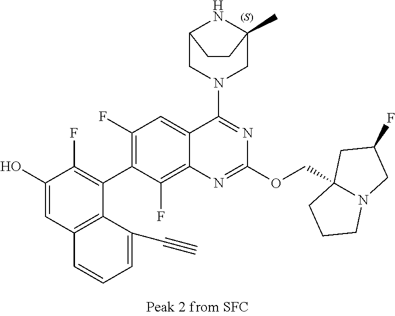 | 4-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan-3- yl)quinazolin-7-yl)-5- ethynyl-3-fluoronaph- thalen-2-ol, 2TFA | 632 | Column B; Condition: 20% IPA w/0.1% NH4OH |
| Ex. 193 | Int 236 Q | 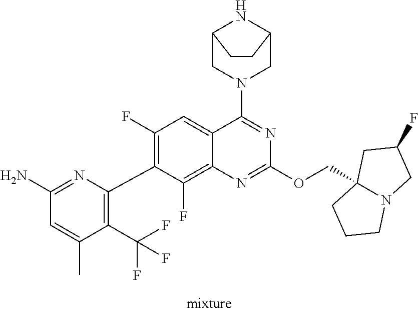 [SMILES: Cc1cc(N)nc(-c2c(F)cc3c(N4CC5CCC(C4)N5)nc(OC[C@@]45CCCN4C[C@H](F)C5)nc3c2F)c1C(F)(F)F] | 6-(4-((1R,5S)-3,8- diazabicyclo[3.2.1]- octan-3-yl)-6,8- difluoro-2-(((2R,7aS)- 2-fluorotetrahydro- 1H-pyrrolizin-7a(5H)- yl)methoxy)quinazo- lin-7-yl)-4-methyl-5- (trifluoromethyl)pyr- idin-2-amine | 608 | -NA- |
| Ex. 194 | 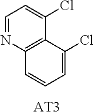 | 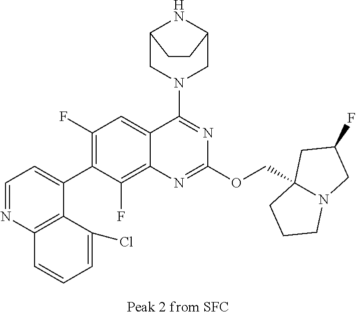 [SMILES: Fc1cc2c(N3CC4CCC(C3)N4)nc(OC[C@@]34CCCN3C[C@H](F)C4)nc2c(F)c1-c1ccnc2cccc(Cl)c12] | 4-((1R,5S)-3,8-diaza- bicyclo[3.2.1]octan-3- yl)-7-(5-chloroquinolin- 4-yl)-6,8-difluoro-2- (((2R,7aS)-2-fluoro- tetrahydro-1H-pyrrol- izin-7a(5H)-yl)meth- oxy)quinazoline | 595 | Column: SJ, 21 × 250 mm, 5 um condition: 20% MeOH w/0.1% NH4OH |
| Ex. 195 | 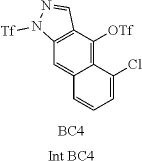 | 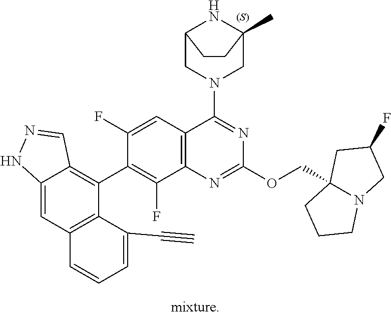 | 4-(6,8-difluoro-2-(((2R, 7aS)-2-fluorotetra- hydro-1H-pyrrolizin- 7a(5H)-yl)methoxy)-4- ((1S,5R)-1-methyl-3,8- diazabicyclo[3.2.1]- octan-3-yl)quinazolin- 7-yl)-5-ethynyl-1H- benzo[f]indazole | 638 | -NA- |
| Ex. 196 | 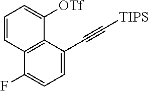 | 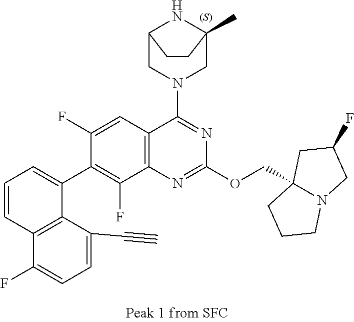 | 7-(8-ethynyl-5-fluoro- naphthalen-1-yl)-6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazoline | 616 | Column: DAICEL CHIRALPAK AC (250 mm * 30 mm, 10 um), Condition: 0.1% NH3H2O ETOH |
| Ex: 197 | 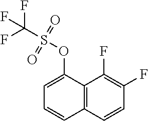 [SMILES: O=S(=O)(Oc1cccc2ccc(F)c(F)c12)C(F)(F)F] | 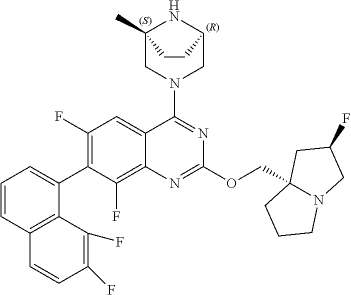 | 7-(7,8-difluoronaph- thalen-1-yl)-6,8- difluoro-2-(((2R,7aS)- 2-fluorotetrahydro-1H- pyrrolizin-7a(5H)-yl)- methoxy)-4-((1S,5R)- 1-methyl-3,8-diaza- bicyclo[3.2.1]octan-3- yl)quinazoline | 610 | Column K; Condition 0.1% NH3H2O IPA |
| Ex. 198 | | 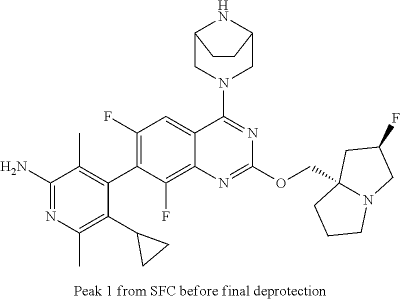 [SMILES: Cc1nc(N)c(C)c(-c2c(F)cc3c(N4CC5CCC(C4)N5)nc(OC[C@@]45CCCN4C[C@H](F)C5)nc3c2F)c1C1CC1] | 4-(4-((1R,5S)-3,8- diazabicyclo[3.2.1]- octan-3-yl)-6,8- difluoro-2-(((2R,7aS)- 2-fluorotetrahydro-1H- pyrrolizin-7a(5H)-yl)- methoxy)quinazolin-7- yl)-5-cyclopropyl-3,6- dimethylpyridin-2- amine | 594 | Column E; condition: 50% MeOH w/0.1% NH4OH |
| Ex. 199 | | 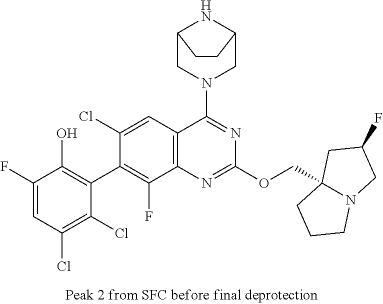 [SMILES: Oc1c(F)cc(Cl)c(Cl)c1-c1c(Cl)cc2c(N3CC4CCC(C3)N4)nc(OC[C@@]34CCCN3C[C@H](F)C4)nc2c1F] | 2-(4-((1R,5S)-3,8- diazabicyclo[3.2.1]- octan-3-yl)-6-chloro-8- fluoro-2-(((2R,7aS)-2- fluorotetrahydro-1H- pyrrolizin-7a(5H)-yl)- methoxy)quinazolin-7- yl)-3,4-dichloro-6- fluorophenol | 628/ 630 | Column A; Condition 0.1% NH₃H₂O IPA |
| Ex. 200a | Int 236 Q | 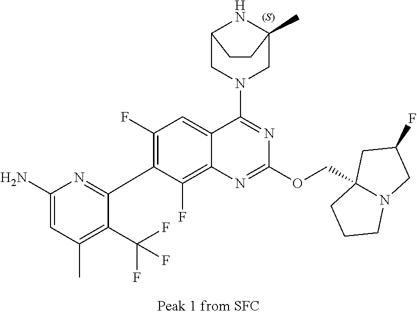 | 6-(6,8-difluoro-2- (((2R,7aS)-2-fluoro- tetrahydro-1H-pyrrol- izin-7a(5H)-yl)meth- oxy)-4-((1S,5R)-1- methyl-3,8-diaza- bicyclo[3.2.1]octan- 3-yl)quinazolin-7-yl)- 4-methyl-5-(trifluoro- methyl)pyridin-2-amine. | 622 | Column: CCU, 21 × 250 mm, 5 um Condition: 35% MeOH w/0.1% NH4OH |
| Ex. 200b | Int 236 Q | 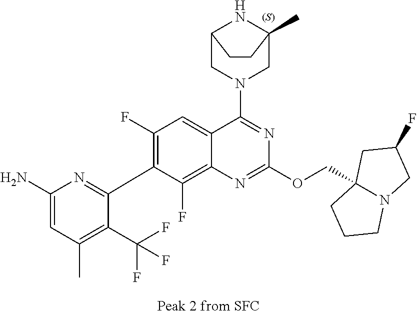 | 6-(6,8-difluoro-2- (((2R,7aS)-2-fluoro- tetrahydro-1H-pyrrol- izin-7a(5H)-yl)meth- oxy)-4-((1S,SR)-1- methyl-3,8-diaza- bicyclo[3.2.1]octan- 3-yl)quinazolin-7-yl)- 4-methyl-5-(trifluoro- methyl)pyridin-2-amine. | 622 | Column: CCU, 21 × 250 mm, 5 um Condition: 35% MeOH w/0.1% NH4OH |
| Ex. 253 | 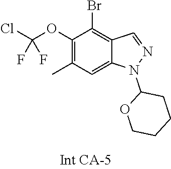 [SMILES: Cc1cc2c(cnn2C2CCCCO2)c(Br)c1OC(F)(F)Cl] | 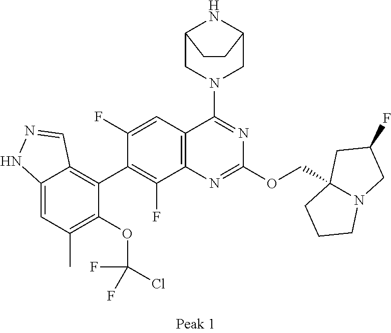 [SMILES: Cc1cc2[nH]ncc2c(-c2c(F)cc3c(N4CC5CCC(C4)N5)nc(OC[C@@]45CCCN4C[C@H](F)C5)nc3c2F)c1OC(F)(F)Cl] | 4-((IR,5S)-3,8-diaza- bicyclo[3.2.1]octan-3- yl)-7-(5-(chlorodi- fluoromethoxy)-6- methyl-1H-indazol-4- yl)-6,8-difluoro-2- (((2R,7aS)-2-fluoro- tetrahydro-1H-pyrrol- izin-7a(5H)-yl)meth- oxy)quinazoline | 664 | Column K; Condition 0.1% NH3H2O ETOH |

### Ex 161. Synthesis of 5-chloro-4-(6,8-difluoro-2-(((2R,7aS)-2-fluorotetrahydro-1H-pyrrolizin-7a(5H)-yl)methoxy)-4-((1R,5S)-3-methyl-3,8-diazabicyclo[3.2.1]octan-3-yl)quinazolin-7-yl)naphthalen-2-ol

### Scheme

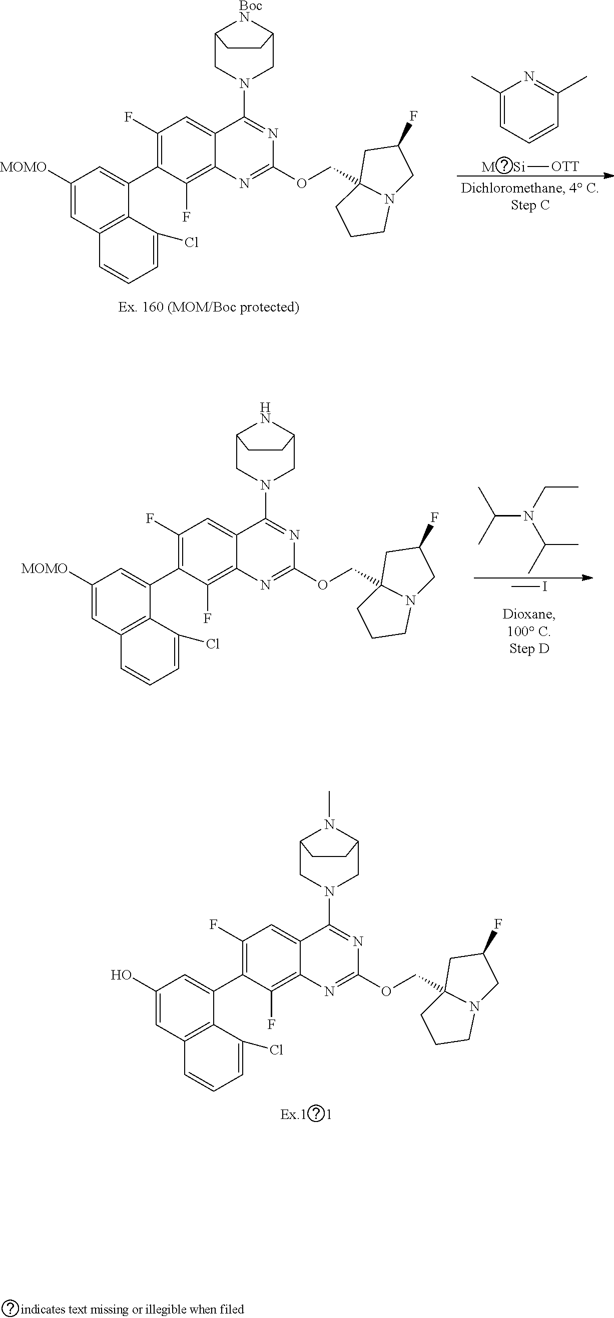

Ex. 160 in the above table/scheme was further processed through the following

### Step C. 4-((1R,5S)-3,8-diazabicyclo[3.2.1]octan-3-yl)-7-(8-chloro-3-(methoxymethoxy)naphthalen-1-yl))-6,8-difluoro-2-(((2R,7aS)-2-fluorotetrahydro-1H-pyrrolizin-7a(5H)-yl)methoxy)Uquinazoline

To a 40 mL glass vial containing tert-butyl (1R,5S)-3-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Ex. 160—MOM/Boc protected) (SFC peak 1) (371 mg, 0.492 mmol) were added a teflon stirbar and anhydrous dichloromethane (2.5 mL), and the contents were mixed to a solution. Then 2,6-dimethylpyridine (172 μL, 1.477 mmol) was added, and the mixture was cooled in an ice/water bath under nitrogen. Then trimethylsilyl trifluoromethanesulfonate (0.18 mL, 0.995 mmol) was added dropwise, and the mixture was stirred at 4° C. for 1 h and 33 min. Then 2M sodium carbonate in water (2.0 mL) was added, and the mixture was diluted with water (4 mL) and mixed. The separated aqueous layer was extracted with dichloromethane (2×2 mL). The combined organic layers were dried over sodium sulfate, filtered and concentrated to afford 4-((1R,5S)-3,8-diazabicyclo[3.2.1]octan-3-yl)-7-(8-chloro-3-(methoxymethoxy)naphthalen-1-yl)-6,8-difluoro-2-((2R,7aS)-2-fluorotetrahydro-1H-pyrrolizin-7a(5H)-yl)methoxy)quinazoline. MS (EST): m/z (M+H)⁺ 654.

### Step D. 5-chloro-4-(6,8-difluoro-2-(((2R,7aS)-2-fluorotetrahydro-1H-pyrrolizin-7a(5H)-yl)methoxy)-4-((1R,5S)-8-methyl-3,8-diazabicyclo[3.2.1]octan-3-yl)quinazolin-7-yl)naphthalen-2-ol (Ex.161)

To a 4 mL glass vial was added 4-((1R,5S)-3,8-diazabicyclo[3.2.1]octan-3-yl)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (20 mg, 0.021 mmol), a teflon stirbar, and 0.21 mL anhydrous dioxane (0.21 mL), and the contents were mixed to a solution. Then N-ethyl-N-isopropylpropan-2-amine (11 μl. 0.063 mmol) was added to the mixture, followed by iodomethane (3.3 μl, 0.053 mmol), and the vial was sealed with a teflon-lined screwcap. The mixture stirred at 100° C. for 9 min, then cooled to 23° C., and allowed to stand at this temperature for 1 h and 40 min, then 4M HCl in dioxane (0.15 mL) was added. The mixture was stirred at 23° C. for 12 min, then ammonia in water (28% ammonia by weight, 0.043 mL) was added to the mixture. After mixing, the mixture was diluted with DMSO (3 mL), filtered through a submicron filter, and the filtrate was purified by HPLC (NH₄OH buffer, ascending gradient of acetonitrile in water) to afford a residue, which was further purified by SFC (Arginine column, 21×250 mm, 5 um, 70 mL/min flow rate, modifier: 40% MeOH w/0.1% NH₄OH). The first peak from SFC purification was collected to afford 5-chloro-4-(6,8-difluoro-2-(((2R,7aS)-2-fluorotetrahydro-1H-pyrrolizin-7a(5H)-yl)methoxy)-4-((1R,5S)-8-methyl-3,8-diazabicyclo[3.2.1]octan-3-yl)quinazolin-7-yl)naphthalen-2-ol. ¹H NMR (499 MHz, DMSO-d₆) δ 10.32 (s, 1H), 7.86 (d, J=8.1 Hz, 1H), 7.63 (m, 1H), 7.45-7.34 (m, 3H), 7.10 (s, 1H), 5.75 (s, 1H), 5.28 (m, 1H), 4.25 (m, 2H), 4.05 (m, 2H), 3.57 (m, 2H), 3.08 (m, 2H), 2.83 (m, 1H), 2.33-1.71 (m, 10H), 1.61 (m, 1H), 1.23 (s, 1H), 0.85 (m, 1H). MS (ESI): m/z (M+H)⁺ 624.

Ex 181: Ex. 89 was carried through the following steps and then followed up with the final steps.

### Intermediate for Ex 181: Tert-butyl (1S,5R)-3-(7-(8-(difluoromethyl)-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3iquiazocl-[3.2.1]-octane-8-carbxcloxy3Int-11P1 and t-xy1P2(

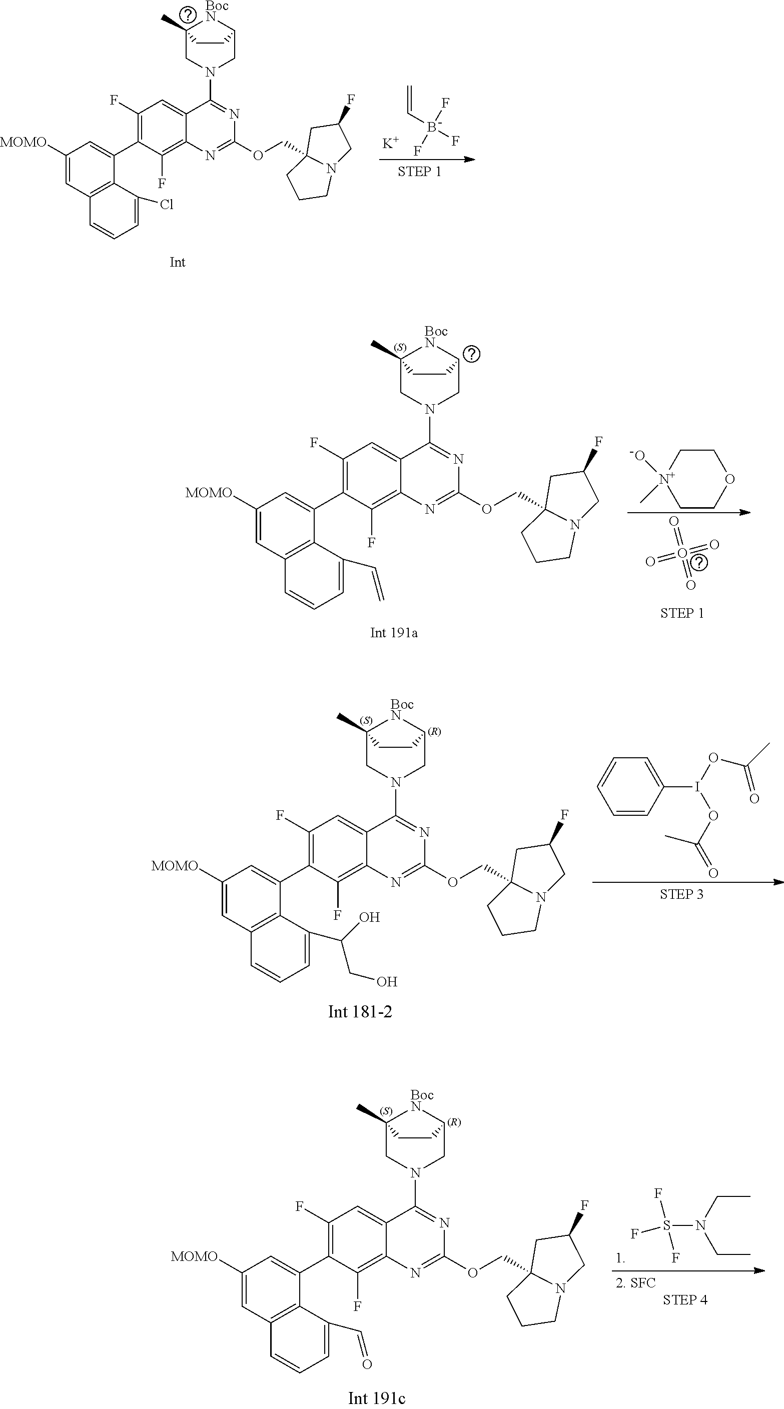

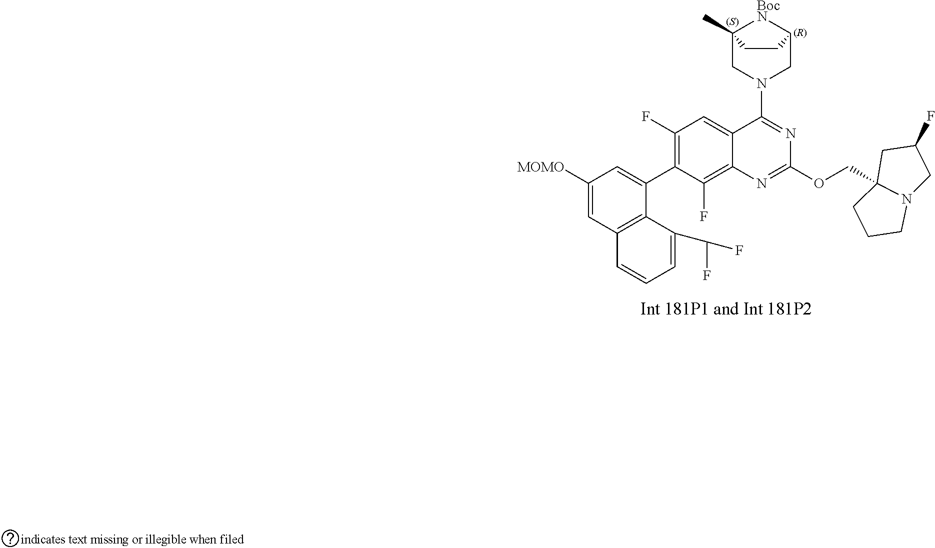

(methmerthoxy)naphthalen-y6,8-difluoro-2-(((2R,7aS)-2-fluorotetraydro-- GP243 1H-pyrrolizin-7a(5H)-yl)methoxy)quinazolin-4-yl)-1-methyl-3,8-diazabicyclo[3.2.1]octane-8-carboxylate (Int-89b, racemic) (180 mg, 0.234 mmol) in DME (2 ml) was added Na₂CO₃ (74.5 mg, 0.703 mmol), potassium vinyltrifluoroborate (157 mg, 1.171 mmol) and (2-Dicyclohexylphosphino-2′,6′-dimethoxybiphenyl) [2-(2′-amino-1,1′-biphenyl)]palladium(II) methanesulfonate (36.6 mg, 0.047 mmol) at 25° C. The reaction was stirred at 100° C. for 16 h under nitrogen atmosphere. The reaction was monitored using LCMS, The reaction was quenched with water (5 mL) and the resulting mixture was extracted with EtOAc (20 mL). The combined organic phase was dried over anhydrous Na₂SO₄, filtered and concentrated in vacuo. The residue was purified by flash silica gel chromatography 4 g column, Pet.ether/EtOAc=0/1, dry loaded, to give tert-butyl (1S,5R)-3-(6,8-difluoro-2-(((2R..7aS)-2-fluorotetrahydro-1H-pyrrolizin-7a(5H)-yl)methoxy)-7-(3-(methoxymethoxy)-8-vinylnaphthalen-1-yl)quinazolin-4-yl)-1-methyl-3,8-diazabicyclo[3.2.1]octane-8-carboxylate. MS (ESI): m/z (M+H)⁺ 760.

### STEP 2: Tert-butyl (1S,5R)-3-(7-(8(1,2-dihydroxyethyl)-3-(methoxymethoxyl) naphthalen-1-yl)-6,8-difluoro-2-(((2R,7aS)-2-fluorotetrahydro-1H-pyrrolizin-7a(5H)-yl)methoxy)quinazolin-4-yl-1-methyl-3,8-diazabicyclo[3.2.1]octane-8-carboxylate (Int-181-2)

To a solution of tert-butyl (1S,5R)-3-(6,8-difluoro-2-(((2R,7aS)-2-fluorotetrahydro-1H-pyrrolizin-7a(5H)-yl)methoxy)-7-(3-(methoxymethoxy)-8-vinylnaphthalen-1-yl)quinazolin-4-yl)-1-methyl-3,8-diazabicyclo[3.2.1]octane-8-carboxylate (60 mg, 0.079 mmol) in t-BuOH (5 ml) and THF (1.5(0) ml)/H₂O (0.500 ml) was added NMO (11.10 mg, 0.095 mmol) and osmium tetroxide (6.02 mg, 0.024 mmol) at 20° C. After addition, the mixture was stirred at 20° C. for 1 h. The reaction was monitored using LCMS, The reaction mixture was diluted with EtOAc(30 mL), quenched with Na₂S2O3 (8 mL). The organic layer was washed with saturated brine (8 mL), dried over Na₂SO₄, filtered and the solvent was evaporated under reduced pressure to give the crude product tert-butyl (1S,5R)-3-(7-(8-(1,2-dihydroxyethyl)-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate which was used directly in the next step without purification. MS (ESI): m/z (M+H)⁺ 794.

### STEP 3: Tert-butyl (1S,5R)-3-(6,8-difluoro-2-(((2S,7aR)-2-fluorotetrahydro-1H-pyrrolizin-7a(5H)-yl)methoxy)-7-(8-formyl-3-(methoxymethoxy)naphthalen-1-yl)quinazolin-4-yl)-1-methyl-3,8-diazabicyclo[3.2.1]octane-8-carboxylate (Int-181c)

To a solution of Tert-butyl (1S,5R)-3-(7-(8-(1,2-dihydroxyethyl)-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-181-2) (62.7 mg, 0.079 mmol) in acetone (5 ml)/H₂O (0.5 ml) was added phenyl-13-iodanediyl diacetate (30.5 mg, 0.095 mmol). The mixture was stirred at 20° C. for 1 h. The reaction was monitored using LCMS, The mixture was diluted with 20 mL EtOAc and washed with sat. Na₂SO₃ (3 mL×2), brine(5 mL), the organic layer was dried and concentrated, the residue was purified by preparative TLC (SiO₂, DCM: MeOH=10:1) to give tert-butyl (1S,5R)-3-(6,8-difluoro-2-(((2S,7aR)-2-fluorotetrahydro-1H-pyrrolizin-7a(5H)-yl)methoxy)-7-(8-formyl-3-(methoxymethoxy)naphthalen-1-yl)quinazolin-4-yl)-1-methyl-3,8-diazabicyclo[3.2.1]octane-8-carboxylate. MS (ESI) m/z: (M+H)⁺ 762.

### STEP 4: Tert-butyl (1S,5R)-347-(84difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-181P1 and Int-181P2)

Step 1: To a solution of tert-butyl (1S,5R)-3-(6,8-difluoro-2-(((2S,7aR)-2-fluorotetrahydro-1H-pyrrolizin-7a(5H)-yl)methoxy)-7-(8-formyl-3-(methoxymethoxy)naphthalen-1-yl)quinazolin-4-yl)-1-methyl-3,8-diazabicyclo[3.2.1]octane-8-carboxylate (Int-181c) (95 mg, 0.125 mmol) in DCM (0.9 ml) was added DAST (0.3 ml, 2.271 mmol) at 20° C. The reaction was stirred at 40° C. for 16 h and monitored by LCMS, The reaction was diluted with EtOAc (20 mL), quenched with NaHCO₃ (aq., 4 mL), the organic layer was dried and concentrated, the residue was purified by Prep-TLC (DCM/MeOH=10:1) to give tert-butyl (1S,5R)-3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate. MS (EST): m/z (M+H)⁺ 784. Resolution of tert-butyl (1S,5R)-3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate and Tert-butyl (1S,5R)-3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate.

Step 2: Racemic tert-butyl 3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate (70 mg, 0.089 mmol) was separated by SFC (Method Comments Column: DAICEL CHIRALPAK IG(250 mm×30 mm, 10 um), condition: 0.1% NH₃H₂O IPA; Begin B 35%: Begin B 35% Gradient Time (min), 100% B Hold Time (min) FlowRate (mL/min) 80 mL/min) to give tert-butyl (1S,5R)-3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate as Peak 1 (P1: the first eluting isomer-Int-181P1) and tert-butyl (1S,5R)-3-(7-(8-(difluoromethyl)-3-(methoxymethoxy)naphthalen-1-yl)-6,8-difluoro-2-(((2S,7aR)-2-fluorotetrahydro-1H-pyrrolizin-7a(5H)-yl)methoxy)quinazolin-4-yl)-1-methyl-3,8-diazabicyclo[3.2.1]octane-8-carboxylate as Peak 2 (P2: the second eluting isomer-Int-181P2). MS (ESI): m/z (M+H)⁺ 784.

### Ex 201: Synthesis of 1-((1R)-1-(((4-(1R,5S)-(3.8-diazabicyclo[3.2.1]octan-3-yl-7-(5-chloro-1-methyl-6-(trifluoromethyl)-1H-indazol-7-yl)-6,8-difluoroquinazolin-2-yl)oxy)methyl)-2,2-difluorocycloprorpyl)-N,N-dimethylmethanamine (Peak B: Ex. 201b)

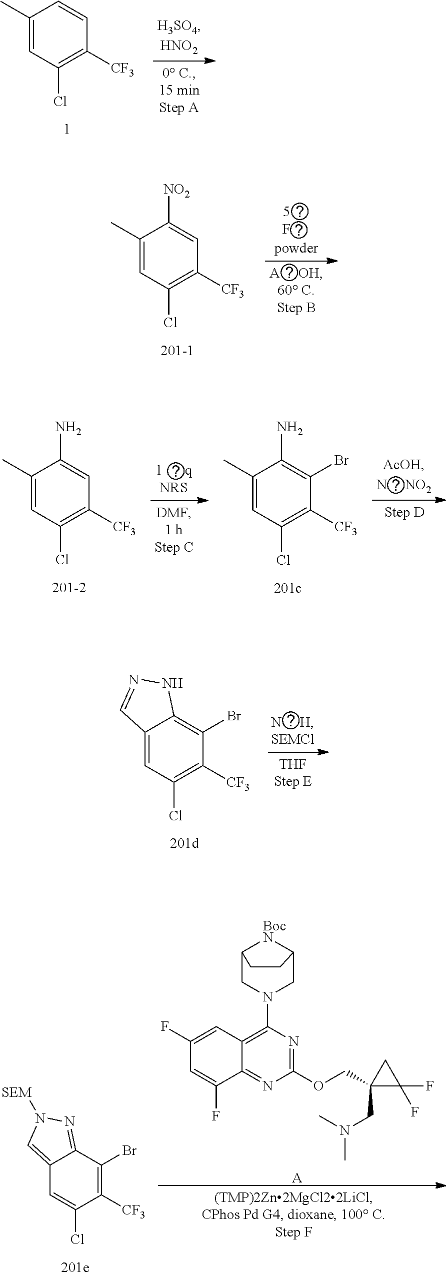

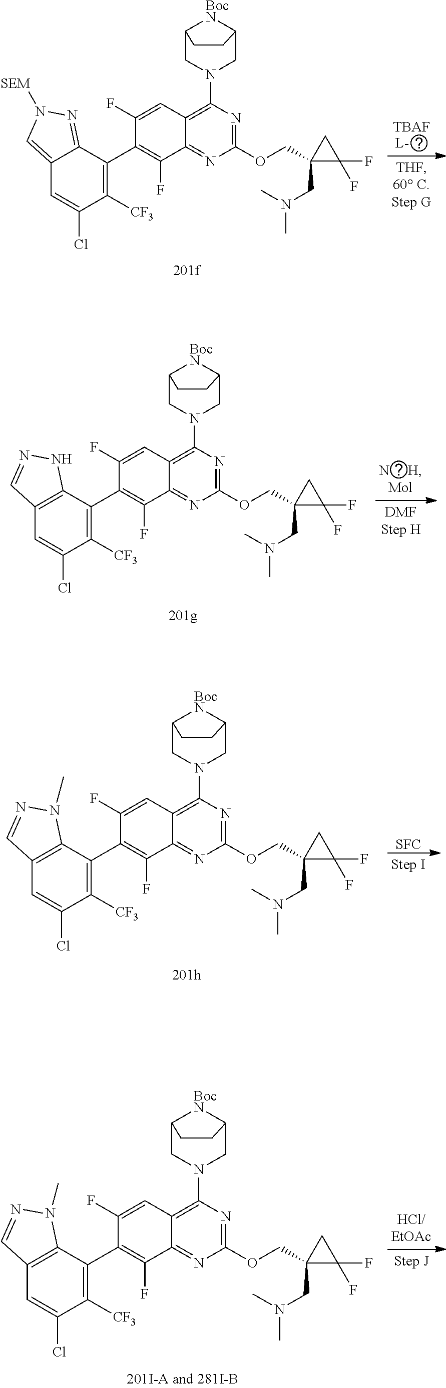

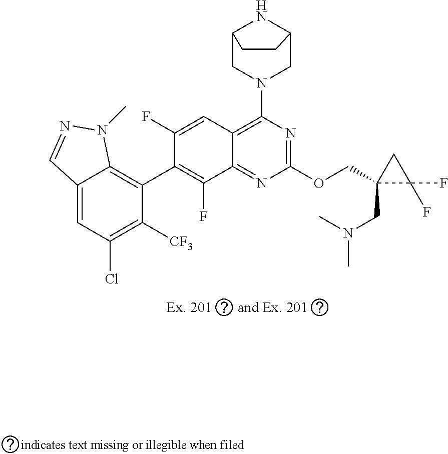

### Step A: 1-chloro-5-methyl-4-nitro-2-(trifluoromethyl)benzene (Int-201-1)

2-chloro-4-methyl-1-(trifluoromethyl)benzene (5 g, 25.7 mmol) was PP25C initially charged into conc. H₂SO₄ (50 mL) and cooled to 0° C., then a mixture of conc. nitric acid (0.857 mL, 25.7 mmol) and conc. H₂SO₄(1,370 mL, 25.7 mmol) were added slowly. The solution was stirred at 0° C. for 15 min. TLC (Pet, ether/EtOAc=5:1) was carried out. The reaction mixture was added to ice-water, and extracted with DCM (100 mL×3), the combined organic phases were dried over MgSO₄ and the solvent was removed under reduced pressure to give the crude product. The crude product was purified by flash silica gel chromatography 24 g Column, eluted with 0-30% ethyl acetate in petroleum ether gradient to give 1-chloro-5-methyl-4-nitro-2-(trifluoromethyl)benzene.

### Step B: 4-chloro-2-methyl-5-(trifluoromethyl)aniline (Int-201-2)

To a solution of I-chloro-5-methyl-4-nitro-2-(trifluoromethyl)benzene (Int-201-1) (5.7 g, 23.79 mmol) in AcOH (60 mL) was added iron powder (6.99 g, 125 mmol). The mixture was stirred at 60° C. for 15 h. The reaction was monitored by LCMS, The mixture was filtered and the solvent was evaporated under reduced pressure to give the crude product. The crude product was purified by flash silica gel chromatography 40 g- Eluent of 0-30% ethyl acetate in petroleum ether gradient to give 4-chloro-2-methyl-5-(trifluoromethyl)aniline. MS (EST): m z (M+H)⁺ +210.

### Step C: 2-bromo-4-chloro-6-methyl-3-(trifluoromethyl)aniline (Int-201c)

To a solution of 4-chloro-2-methyl-5-(trifluoromethyl)aniline (Int-201-2) (4.3 g, 20.52 mmol) in DMF (50 mL) was added 1-bromopyrrolidine-2,5-dione (3.65 g, 20.52 mmol) at 0° C. The mixture was warmed to 25° C., and stirred for 1 h. The reaction was monitored by LCMS, TLC (Pet, ether/EtOAc=3:1) was carried out. The mixture was diluted with water (60 mL) and extracted with EtOAc (100 mL×3). The combined organic layers were washed with brine (50 mL), dried over Na₂SO₄, filtered, and concentrated under reduced pressure to give the crude product, which was purified by flash silica gel chromatography 40 g column, Eluent of 0-30% ethyl acetate in petroleum ether gradient®2: 40 mL/min) to give 2-bromo-4-chloro-6-methyl-3-(trifluoromethyl)aniline. MS (ESI): m/z (M+H)⁺ 288, 290.

### Step D: 7-bromo-5-chloro-6-(trifluoromethyl)-1H-indazole (Int-201d)

To a solution of 2-bromo-4-chloro-6-methyl-3-(trifluoromethyl)aniline (Int-201c) (4.3 g, 14.91 mmol) in AcOH (50 mL) was added sodium nitrite (1.131 g, 16.40 mmol). The mixture was stirred at 50° C. for 15 h. Reaction was monitored by LCMS, The solvent of the reaction mixture was evaporated under vacuum. The crude product was basified with NaHCO₃(aq) to pH 8. The aqueous layer was extracted with EtOAc (100 mL×3). The combined organic layers were dried over MgSO₄, filtered and the solvent was evaporated under reduced pressure. The crude product was purified by flash silica gel chromatography 40 g column, Eluent of 0-30% ethyl acetate in petroleum ether gradient @40 mL/min) to give 7-bromo-5-chloro-6-(trifluoromethyl)-1H-indazole. MS (ESI): m/z (M+H)⁺ 299, 301.

### Step E: 7-bromo-5-chloro-6-(trifluoromethyl)-2-((2-(trimethylsilyl)ethoxy) methyl)-2H-indazole (Int-201e)

To a solution of 7-bromo-5-chloro-64trifluoromethyl)-1H-indazole (Int-201d) (1.9 g, 6.34 mmol) in THF (20 mL) was added NaH (0.534 g, 13.36 mmol) (60% in mineral oil) at 0° C. and stirred for 20 min. Then (2-(chloromethoxy)ethyl)trimethylsilane (1.777 mL, 10.02 mmol) was added to the above solvent. The mixture was stirred at 25° C. for 3 h. The reaction was monitored by LCMS, The reaction was concentrated in vacuo to give the crude product. The crude product was purified by flash silica gel chromatography 40 g column, Eluent of 0-20% EtOAc/Pet, ether gradient @, 40 mL/min to give 7-bromo-5-chloro-6-(trifluoromethyl)-2-((2-(trimethylsilyl)ethoxy)methyl)-2H-indazole (Int-2Ile) and 7-bromo-5-chloro-6-(trifluoromethyl)-1-((2-(trimethylsilyl)ethoxy)methyl)-1H-indazole. MS (ESI): m/z (M+H)⁺ 429, 431. ¹H NMR (400 MHz, CDCl₃) 58.24 (s, 1H), 7.84 (s, 1H), 5.79 (s, 2H), 3.70-3.63 (m, 2H), 0.99-0.92 (m, 2H), -0.02 (s, 9H).

### Step F: Tert-butyl (1R,5S)-3-(7-(5-chloro-64trifluoromethyl)-2-((2-(trimethylsilyl)ethoxy)methyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-201f)

To a solution of tert-butyl 3-(2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-C₄) (250 mg, 0.463 mmol) in dry THF (0.2 mL) was added (TMP)₂Zn·2MgCl₂·LiCl (4.21 mL, 1.390 mmol) (0.33M in THF) dropwise at 25° C. under N₂ atmosphere. After addition, the mixture was stirred at 50° C. for 16 h. A solution of 7-bromo-5-chloro-6-(trifluoromethyl)-2-((2-(trimethylsilyl)ethoxy)methyl)-2H-indazole (Int-211e) (239 mg, 0.556 mmol) and CPhos Pd G4 (76 mg, 0.093 mmol) in dioxane (0.2 mL) was added to the above solution in glove box, after addition, the reaction was stirred at 100° C. for 12 h. The reaction was monitored by LCMS, The reaction mixture was added to water (10 mL), extracted with ethyl acetate (30 mL×2), dried over Na₂SO₄, filtered and the solvent was evaporated under reduced pressure to give the crude product, which was purified by preparative TLC plate (SiO₂, DCM/MeOH=10/1) to give tert-butyl (1R,5S)-3-(7-(5-chloro-6-(trifluoromethyl)-2-((2-(trimethylsilyl)ethoxy)methyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate. MS (ESI): m/z (M+H)⁺ 888.

### Step G: Tert-butyl (1R,5S)-3-(7-(5-chloro-64trifluoromethyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-201g)

To a solution of tert-butyl (1R,5S)-3-(7-(5-chloro-6-(trifluoromethyl)-2-((2-(trimethylsilyl)ethoxy)methyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-201) (200 mg, 0.225 mmol) in TBAF (3 mL, 3.00 mmol) (IM in THF) was added L-cysteine (54.5 mg, 0.450 mmol). After addition, the mixture was stirred at 50° C. for 3 h. The reaction was monitored by LCMS, The reaction was concentrated in vacuo to get the crude product, which was purified by preparative HPLC (Phenomenex Synergi C18 150*21.2 mm*4 um Condition water (0.1% TFA)-ACN/Water to give tert-butyl (1R,5S)-3-(7-(5-chloro-6-(trifluoromethyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate. MS (ESI): m/z (M+H)⁺ 758.

### Step H: Tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-201h)

To a solution of tert-butyl (1R,5S)-3-(7-(5-chloro-6-(trifluoromethyl)-2H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-201g) (110 mg, 0.145 mmol) in dry DMF (1 mL) was added NaH (17.41 mg, 0.435 mmol) (60% in mineral oil) at 0° C. under N₂. The reaction was stirred for 10 min. Then Mel (9.07 μl, 0.145 mmol) was added into the reaction mixture. The reaction was monitored by LCMS, The reaction mixture was quenched with water (1 mL), then the mixture was extracted with EtOAc (4 mL×3), the combined organic layer was washed with brine (5 mL), dried over anhydrous Na₂SO₄, filtered and concentrated in vacuum to give crude product, which was purified by (Boston Green ODS 150*30 mm*5 um Condition water (0.1% TFA)-ACN -Water to give tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate. MS (ESI): m/z (M+H)⁺ 772. ¹H NMR (400 MHz, MeOD) δ 8.23 (s, 1H), 8.19-8.13 (s, 1H), 7.81-7.67 (d, 1H), 4.56 (s, 2H), 4.49 (br dd, J=6.8, 11.9 Hz, 2H), 4.38 (br s, 2H), 3.65 (br d, J=11.7 Hz, 2H), 3.45 (d, J=1.6 Hz, 3H), 2.96-2.75 (m, 1H), 2.63-2.40 (m, 1H), 2.26 (s, 6H), 1.99-1.86 (m, 4H), 1.72-1.61 (m, 1H), 1.52 (s, 9H), 1.43-1,34 (m, 1H).

### Step 1: Tert-butyl (0R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluorouquinazolin-4-yl)-3,8-diazabicyclo[3.2.1]octane-8-carboxylate (Int 2011-A &Int 2011-B)

The racemic tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-y)-3,8-diazabicyclo[3.2.1]octane-8-carboxylate (110 mg, 0.142 mmol) was separated by SFC Column E; Condition 0.1% NH₃H₂O EtOH to give tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int 2011-A), the first eluting isomer) and tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-2011-B), the second eluting isomer) both as colorless oil. MS (ESI): m/z (M+H)⁺ 772.

### Step J: 1-((1R)-1-(((4-((1R,5S)-3,8-diazabicyclo[3.2.1]octan-3-yl-7-(5-chloro-1-methyl-6-(trifluoromethyl)-1H-indazol-7-yl)-6,8-difluoroquinazolin-2-yl)oxy)methyl)-2,2-difluorocyclopropyl)-N,N-dimethylmethanamine -Peak A-(Ex.201A)

Tert-butyl (1R,5S)-3-(7-(5-chloro-1-methyl-6-(trifluoromethyl)-1H-indazol-7-yl)-2-(((R)-1-((dimethylamino)methyl)-2,2-difluorocyclopropyl)methoxy)-6,8-difluoroquinazolin-4-yl)-3,8-diazabicyclo[3.2.1]octane-8-carboxylate (Int-2011-A) (15 mg, 0.019 mmol) was added to HCl/EtOAc (0.5 mL) (4M), and the mixture was stirred at 15° C. for 30 min. The reaction was monitored by LCMS, The reaction was concentrated under vacuum to get the crude product, which was purified by pre-HPLC (Column YMC-Actus Triart C18 100*30 mm*5 um Condition water (0.1% TFA)-ACN to give 1-((1R)-1-(((4-((1R,5S)-3,8-diazabicyclo[3.2.1]octan-3-yl)-7-(5-chloro-1-methyl-6-(trifluoromethyl)-1H-indazol-7-yl)-6,8-difluoroquinazolin-2-yl)oxy)methyl)-2,2-difluorocyclopropyl)-N,N-dimethylmethanamine. MS (ESI): m: (M+H)⁺ 672. Peak A (Ex.201A): ¹H NMR (400 MHz, MeOD) δ 8.27 (s, 1H), 8.20 (s, 1H), 7.81 (d, J=8.6 Hz. 1H). 4.79-4.72 (m, 1H), 4.70-4.58 (m, 3H). 4.25 (br s, 2H), 3.86 (br d, J=14.5 Hz, 2H), 3.82-3.73 (m, 1H), 3.46 (s, 4H), 3.02 (s, 6H), 2.27-2.10 (m, 4H), 2.06-1.91 (m, 1H), 1.88-1.76 (m, 1H). The procedure of Peak B (using Int-2011-B) was similar as Peak A and the spectrum is as shown below: Peak B (Ex.201B): ¹H NMR (400 MHz, MeOD) δ 8.27 (s, 1H), 8.20 (s, 1H), 7.81 (d, J=8.2 Hz, 1H), 4.81 (br d, J=12.1 Hz, 1H), 4.71-4.53 (m, 3H), 4.25 (br s, 2H), 3.85 (br d, J=14.1 Hz, 2H), 3.74 (br d, J=14.1 Hz. 1H), 3.55-3.41 (m, 4H), 3.02 (s, 6H), 2.33-2.11 (m, 4H), 2.09-1.92 (m, 1H), 1.90-1.76 (m, 1H). MS (ESI): m/z (M+H)⁺ 672.

### Example 202: 4-(6,8-difluoro-2-(((2R,7aS)-2-fluorotetrahydro-1H-pyrrolizin-7a(5f)-yl)methoxy)-4-((1S,5R)-1-methyl-3,8-diazabicyclo[3.2.1]octan-3-yl)quinazolin-7-yl)-5-ethynyl-1-fluoronaphthalen-2-ol

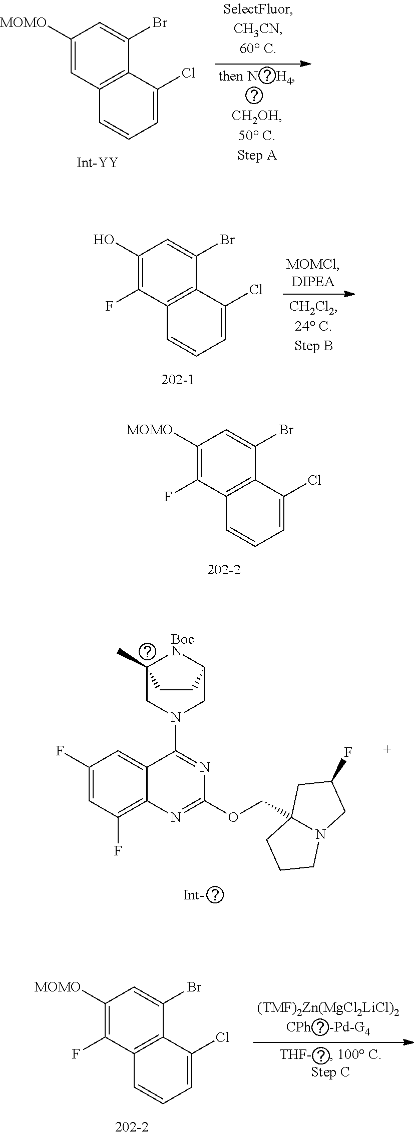

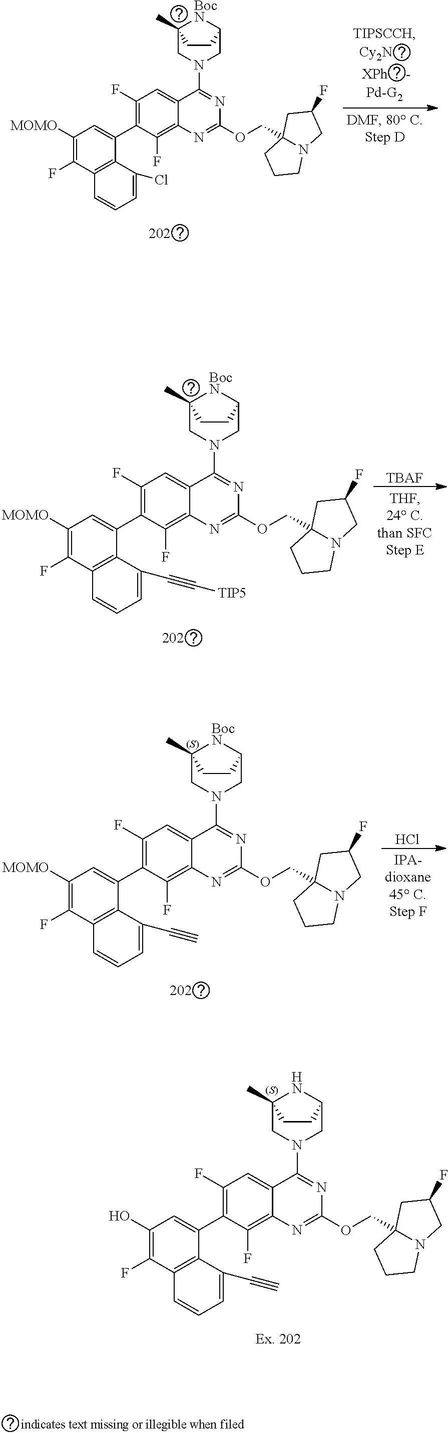

### Step A: 4-bromo-5-chloro-1-fluoronaphthalen-2-ol (Int 202-1)

1-Bromo-8-chloro-3-(methoxymethoxy)naphthalene (Int-III4) (300 mg. 0.995 mmol) was added in a 30 mL vial. The reaction vessel was evacuated and backfilled with a balloon of nitrogen three times. Acetonitrile (3.3 mL) and 1-chloromethyl-4-fluoro-1,4-diazoniabicyclo[2.2.2]octane bis(tetrafluoroborate) (705 mg, 1.99 mmol) were added into the reaction vessel. The resulting mixture was heated at 60° C. for 2h. The resulting mixture was cooled down to room temperature. The cooled mixture was diluted with ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was dissolved in methanol (3.3 mL) under nitrogen. The resulting mixture was cooled down to 0° C. Then 1.8-diazabicyclo[5.4.0]undec-7-ene (757 mg, 4.97 mmol) and sodium tetrahydroborate (188 mg, 4.97 mmol) were added into the reaction vessel. The resulting mixture was stirred for 5 min at 0° C. The resulting mixture was heated to 50° C. for 30 min. The product mixture was quenched with saturated ammonium chloride aqueous mixture (5 mL). The quenched product mixture was diluted with ether (100 mL) and acidified with 3N HCl (5 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford the desired product 4-bromo-5-chloro-1-fluoronaphthalen-2-ol. ¹H NMR (499 MHz, Acetonitrile-d₃) δ 8.04 (d, J=8.5 Hz, 1H), 7.79 (s, 1H), 7.75 (d, J=8.5 Hz, 1H), 7.63 (d, J=7.4 Hz, 1H), 7.50 (t, J=8.0 Hz, 1H).

Step B: 4-bromo-5-chloro-1-fluoro-2-(methoxymethoxy)naphthalene (Int-202-4-Bromo-5-chloro-1-fluoronaphthalen-2-ol (Int-202-1) (130 mg, 0.472 mmol) was dissolved in DCM (1.6 mL). N-Ethyl-N-isopropylpropan-2-amine (336 μL, 1.89 mmol) and chloro(methoxy)methane (76 μL, 0.94 mmol) were added into the reaction vessel. The resulting mixture was stirred for 15 min at 24° C. The product mixture was diluted with ether (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 20% ethyl acetate-hexanes, linear gradient to afford 4-bromo-5-chloro-1-fluoro-2-(methoxymethoxy)naphthalene. ¹H NMR (6O MHz, Acetonitrile-d₃) δ 8.10 (dd, J=8.5, 1.2 Hz, 1H), 7.98 (d, J=8.2 Hz. 1H). 7.70 (dd, J=7.5, 1.2 Hz, 1H), 7.53 (dd, J=8.5, 7.4 Hz, 1H), 5.35 (s, 2H), 3.54 (s, 3H).

### Step C: Tert-butyl 3-(7-(8-chloro-4-fluoro-3-(methoxymethoxy)naphthalen-1-yl-6,8-difluoro-2-(((2R,7aS)-2-fluorotetrahydro-1H-pyrrolizin-7a(5H)-yl)methoxy)quinazolin-4-yl(1S,5R)-1-methyl-3,8-diazabicyclo[3.2.1]octane-8-carboxylate (Int-202c)

In the glovebox, (TMP)₂Zn(MgCl₂LiCl) (1,34 mL, 0.201 mmol) was added in a 30 mL vial. In a separate vail, tert-butyl (I S,5R)-3-(6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89a) (100 mg. 0.183 mmol) was dissolved in THF (1 mL). The starting material solution was added dropwise into the zinc reagent. The starting material vessel was washed three times with THF (3; ×300 uL) and the combined rinse was added dropwise into the reaction vessel. The resulting mixture was stirred for 1 h at 24° C. In a third vial. 4-bromo-5-chloro-1-fluoro-2-(methoxymethoxy)naphthalene (Int-202-2) (II1 mg, 0.347 mmol) and CPhos Pd G4 (23 mg, 0.027 mmol) were dissolved in dioxane (1 mL). This solution was added dropwise into the reaction vessel. The vessel containing the electrophile was rinsed with dioxane (3-300 μL). The combined rinses were added dropwise into the reaction vessel. The reaction vessel was removed from the glovebox and then heated at 100° C. for 2h. The product mixture was cooled down to room temperature and diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 100% ethyl acetate: ethanol=3:1, linear gradient to afford tert-butyl 347-(8-chloro-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate. MS (ESI): m/z (M+H)⁺ 786. Step D: Tert-butyl 3-(6,8-difluoro-7-(4-fluoro-3-(methoxymethoxy)-8-((triisonrooylsilyl)ethynyl)naphthalen-1-yl)-2-(((2R,7aS)-2-fluorotetrahydro-1H-pyrrolizin-7a(5H)-yl)methoxy)quinazolin-4-yl)-(1S,5R)-methyl-3,8-diazabicyclo[3.2.1]octane-8-carboxylate (Int 202d)

Tert-Butyl 3-(7-(8-chloro-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate (Int-202c) (110 mg, 0.140 mmol), ethynyltriisopropylsilane (314 μL, 1.40 mmol), Xphos-Pd-G3 (59 mg, 0.070 mmol), N-cyclohexyl-N-methyl)cyclohexanamine (137 mg, 0.700 mmol) were added into a 20 mL vial. The reaction vessel was evacuated and backfilled with a balloon of nitrogen three times. DMF (1.4 mL) was added into the reaction vessel. The resulting mixture was heated at 80° C. for 16h. The product mixture was diluted with ethyl acetate (100 mL). The diluted product mixture was washed three times with saturated sodium bicarbonate aqueous solution (3-20 mL). The organic layer was dried over sodium sulfate. The dried solution was filtered and the filtrate was concentrated to dryness. The residue obtained was purified by flash column chromatography with 12-g amino-functionalized silica gel column, eluting with hexanes initially, grading to 50% ethyl acetate-hexanes, linear gradient to afford the desired product tert-butyl 3-(6,8-difluoro-7-(4-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)- (1 S,5R)-methyl-3,8-diazabicyclo[3.2.1]octane-8-carboxylate. MS (ESI): m/z (M+H)⁺ 932.

### Step E: Tert-butyl 3-(7-(8-ethynyl-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl(1S,5R)-1-methyl-3,8-diazabicyclo[3.2.1]octane-8-carboxylate (Int-202e)

Tert-Butyl 3-(6,8-difluoro-7-(4-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate (Int-202d) (124 mg, 0.113 mmol) was added in a 20 mL vial. The reaction vessel was evacuated and backfilled with a balloon of nitrogen three times. THF (1.1 mL) was added into the reaction vessel. Tetrabutylammonium fluoride (113 μL, 0.113 mmol) was added into the reaction vessel. The resulting mixture was stirred for 5 min. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 100o ethyl acetate: ethanol=3:1, linear gradient. The racemic material was further resolved by SFC (AD-H, 21×250 mm, 5 um; Flow rate: 70 mL/min; modifier: 20% IPA w/0.1% NH₄OH) to afford tert-butyl 3-(7-(8-ethynyl-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate (Int-202e) as Peak 1, RT=3.50 mins. MS (ESI): m/z (M+H)⁺ 776, and tert-butyl 3-(7-(8-ethynyl-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate as Peak 2, RT=4.46 mins. MS (ESI): m/z (M+H)⁺ 776. Step F: 4-(6,8-difluoro-2-(((2R,7aS)-2-fluorotetrahydro-1H-pyrrolizin-7a(5H)-yl)methoxy)-4-((1.5R)-1-methyl-3,8-diazabicyclo[3.2.1]octan-3-yl)quinazolin-7-yl)-5-ethynyl-1-fluoronaphthalen-2-ol (Ex-202a)

Tert-Butyl 3-(7-(8-ethynyl-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate (25 mg, 0.032 mmol) was added in an 8 mL vial. The reaction vessel was evacuated and backfilled with a balloon of nitrogen three times. 2-Propanol (710 μL) was added into the reaction vessel to dissolve the starting material. Then 4N HCl in dioxane (355 μL) was added dropwise. The resulting mixture was heated at 45° C. for 1 h. The product mixture was concentrated to dryness. The residue obtained was purified by Singleton. The dried down fraction was re-lyophylzed to afford the desired product 4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-5-ethynyl-1-fluoronaphthalen-2-ol. MS (ESI): m/z (M+H)⁺ 632. ¹H NMR (500 MHz, DMSO-do) δ 10.85 (s, 1H), 10.71 (s, 1H), 9.46-8.90 (m, 2H), 8.12 (dd, J=7.1, 2.5 Hz, 1H), 7.71 (d, J=9.8 Hz, 1H), 7.61 (q, J=4.8 Hz, 2H), 7.27 (d, J=8.4 Hz, 1H), 5.58 (d.. =52.4 Hz, 1H), 4.58 (q, J=11.9 Hz, 3H), 4.34 (d, J=13.7 Hz, 1H), 4.25 (s, 1H), 4.00-3.62 (m, 4H), 2.60 (d, J=40.0 Hz, 4H), 2.44-2.30 (m, 1H), 2.26-1.99 (m, 7H), 1.75 (d, J=12.5 Hz, 1H), 1.50 (s, 3H).

### Step G: 4-68-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-5-ethynyl-1-fluoronaphthalen-2-ol (Ex.202b)

Tert-Butyl 3-(7-(8-ethynyl-4-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-(1S,5R)-1-methyl-3,8-diazabicyclo[3.2.1]octane-8-carboxylate- Peak 2 from Step E was similarly processed as STEP F to give the title compound. MS (ESI): m/z (M+H)⁺ 632. ¹H NMR (500 MHz, DMSO-d₆) S 10.85 (s, 1H), 10.71 (s, 1H), 9.46-8.90 (m, 2H), 8.12 (dd, J=7.1, 2.5 Hz, 1H), 7.71 (d, J=9.8 Hz, 1H), 7.61 (q, J=4.8 Hz, 2H), 7.27 (d, J=8.4 Hz, 1H), 5.58 (d, J=52.4 Hz, 1H), 4.58 (q, J=11.9 Hz, 3H), 4.34 (d, J=13.7 Hz, 1H), 4.25 (s, 1H), 4.00-3.62 (m, 4H), 2.60 (d, J=40.0 Hz, 4H), 2.44-2.30 (m, 1H), 2.26-1.99 (m, 7H), 1.75 (d, J=12.5 Hz, 1H), 1.50 (s, 3H).

The compounds in the table below were synthesized following a similar procedure using intermediates described in other sections.

| | | | | | SFC |
|---|---|---|---|---|---|
| | Starting | | | | Purification |
| Ex. | Material | Structure | Compound Name | [M + H]⁺ Found | Conditions |
| Ex. 203a | 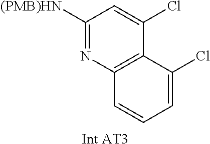 | 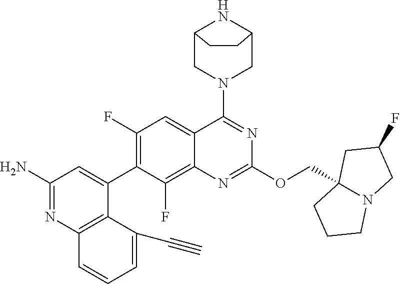 [SMILES: C#Cc1cccc2nc(N)cc(-c3c(F)cc4c(N5CC6CCC(C5)N6)nc(OC[C@@]56CCCN5C[C@H](F)C6)nc4c3F)c12] | 4-(4-((1R,5S)-3,8-diazabicyclo[3.2.1]octan- 3-yl)-6,8-difluoro-2-(((2R,7aS)-2-fluo- rotetrahydro-1H-pyrrolizin-7a(5H)-yl)meth- oxy)quinazolin-7-yl)-5-ethynylquinolin- 2-amine | 600 | Column L; Condition: 50% MeOH w/0.1% NH4OH |
| | | Peak 1 from SFC before final | | | |
| | | deprotection | | | |
| Ex. 203b | 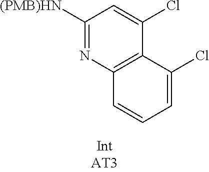 | 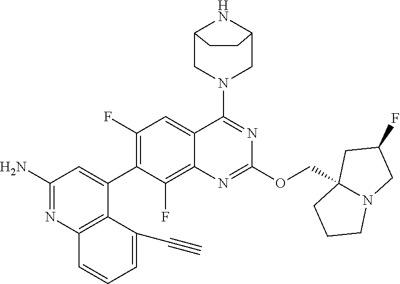 [SMILES: C#Cc1cccc2nc(N)cc(-c3c(F)cc4c(N5CC6CCC(C5)N6)nc(OC[C@@]56CCCN5C[C@H](F)C6)nc4c3F)c12] | 4-(4-((1R,5S)-3,8-diazabicyclo[3.2.1]octan- 3-yl)-6,8-difluoro-2-(((2R,7aS)-2-flou- rotetrahydro-1H-pyrrolizin-7a(5H)-yl)meth- oxy)quinazolin-7-yl)-5-ethynylquinolin- 2-amine | 600 | Column L; Condition: 50% MeOH w/0.1% NH4OH |
| | | Peak 2 from SFC before final | | | |
| | | deprotectoin | | | |
| Ex. 204a | 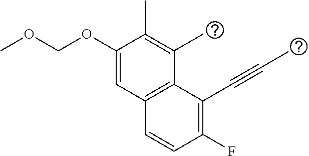 [SMILES: CC#Cc1c(F)ccc2cc(OCOC)c(C)c(C)c12] | 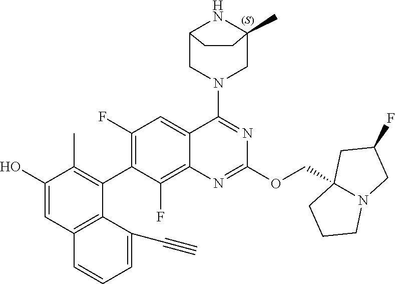 | 4-(6,8-difluoro-2-(((2R,7aS)-2- fluorotetrahydro-1H-pyrrolizin-7a(5H)- yl)methoxy)-4-((1S,4R)-1-methyl-3,8- diazabicyclo[3.2.1]octan-3-yl)quinazolin- 7-yl)-5-ethynyl-3-methylnaphthalen-2-ol | 628 | Column: DAICEL CHIRALCEL OJ (250 mm × 30 mm, 10 um); Condition: 0.1% NH₃H₂O IPA |
| | | Peak | | | |
| | | 1 from SFC before final | | | |
| | | deprotection | | | |
| Ex. 204b | 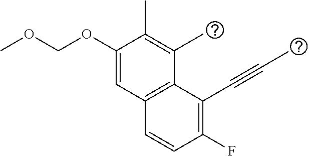 [SMILES: CC#Cc1c(F)ccc2cc(OCOC)c(C)c(C)c12] | 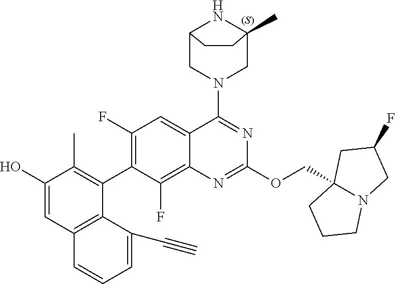 | 4-(6,8-difluoro-2-(((2R,7aS)-2- fluorotetrahydro-1H-pyrrolizin-7a(5H)- yl)methoxy)-4-((1S,4R)-1-methyl-1,3,8- diazabicyclo[3.2.1]octan-3-yl)quinazolin- 7-yl)-5-ethynyl-3-methylnaphthalen-2-ol | 628 | Column: DAICEL CHIRALCEL OJ (250 mm * 30 mm, 10 um): Condition: 0.1% NH₃H₂O IPA |
| | | Peak 2 from SFC before final | | | |
| | | deprotection | | | |

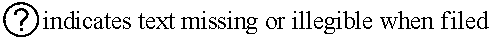

### Example 90: 4-(2-(((R)-2,2-difluoro-1-(hydroxymethyl)cyclopropyl)methoxy)-6,8-difluoro-4-((1S,5R)-1-methyl-3,8-diazabicyclo[3.2.1]octan-3-yl))quinazolin-7-yl)-

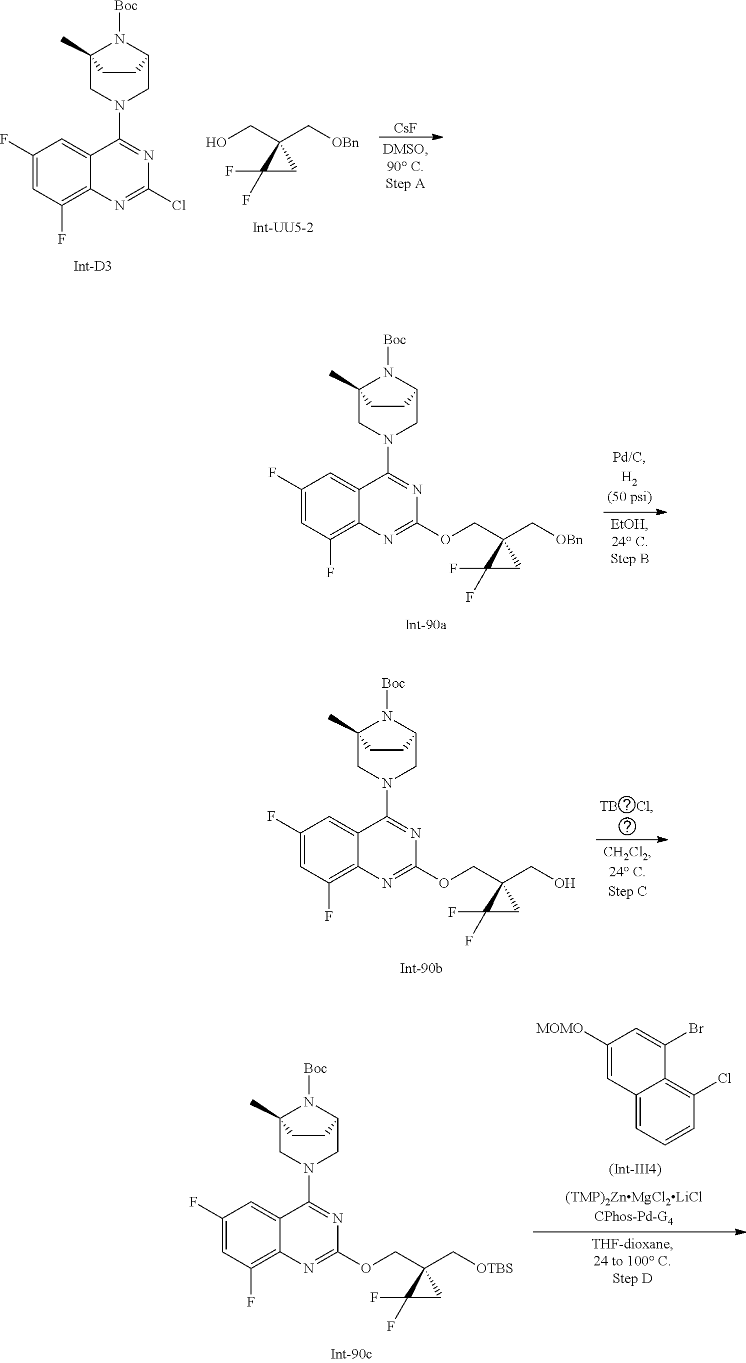

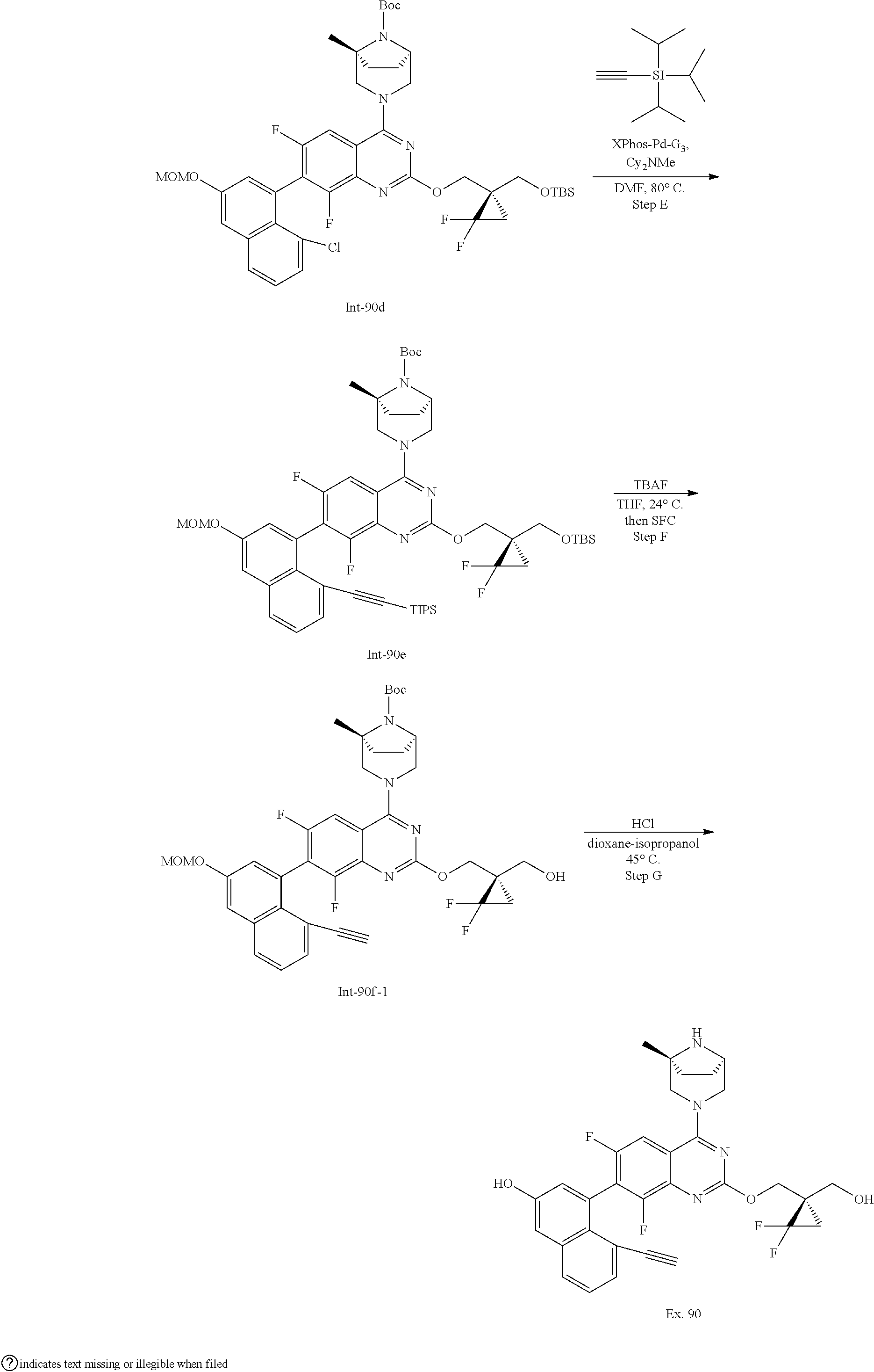

### Step 5 A: Tert-butyl (1S,5R)-3-(2-(((R)-2-methoxy)metloy-2,2 difluorocyclopropyl)methoxy,)-6,8-difluoroquinazolin-4-yl)-1-methyl-3,8-diazabicyclo[3.2.1]octane-8-carboxylate (Int-90a)

To a solution of tert-butyl (1S,5R,)-3-(2-chloro-6,8-difluoroquinazolin-4-yl)-1-methyl-3,8-diazabicyclo[3.2.1]octane-8-carboxylate (Int-D3, 465 mg, 1.10 mmol) in DMSO (2.7 mL) were added (S)-(1-((benzyloxy)methyl)-2,2-difluorocyclopropyl)methanol (Int-UU5-2) (300 mg, 1.31 mmol) followed by cesium fluoride (832 mg, 5.48 mmol). The reaction mixture was purged with nitrogen for 5 mins and heated at 100° C. for 16 h. The product mixture was cooled to 24′° C. Water was added into the product mixture and solid precipitated. The solid was filtered and dried under vacuum. The crude product was purified by column chromatography (Eluent of 35--100% EtOAc in hexanes) to give terl-butyl (1 S, S)-3-(24((R)-1-((benzyloxy)methyl)-2,2-difluorocyclopropyl)methoxy)-6,8-difluoroquinazolin-4-yl)-1-methyl-3,8-diazabicyclo[3.2.1]octane-8-carboxylate (Int-90a). MS (EST): m/z (M+H)⁺ 617.

### Step B: Tert-butyl (1S,5R)-3-(2-(((R)-2,2-difluoro-1-(hydroxymethyl)cyclopropyl)methoxy)-6,8-difluoroquinazolin-4-yl)-1-methyl-3,8-diazabicyclo[3.2.1]octane-8-carboxylate (Int-90b)

Tert-Butyl (1.S,5R)-3-(2-(((R)-1-((benzyloxy)methyl)-2,2-difluorocyclopropyl)methoxy)-6,8-difluoroquinazolin-4-yl)-1-methyl-3,8-diazabicyclo[3.2.1]octane-8-carboxylate (Int-90a, 612 mg, 0.992 mmol), palladium on activated carbon (106 mg, 0.099 mmol), and ethanol (9.9 mL) were added in a 100 mL Parr shaker vessel. The resulting mixture was shook at 24° C. under 50 psi of hydrogen gas for 24 h. The product mixture was purged thoroughly and filtered. The filtrate was concentrated to dryness. The residue obtained was purified by flash column chromatography with 12-g silica gel column, eluting with hexanes initially, grading to 40% ethyl acetate/ethanol (3:1, v/v) in hexanes, linear gradient to afford tert-butyl (1S,5R)-3-(2-(((R)-2,2-difluoro-1-(hydroxymethyl)cyclopropyl)methoxy)-6,8-difluoroquinazolin-4-yl)-1-methyl-3,8-diazabicyclo[3.2.1]octane-8-carboxylate (Int-90b). MS (ESI): m/z (M+H)⁺ 527.

### Step C: Tert-butyl (1S,5R)-3-(2-(((S)-1-(((tert-butyldimethylsilyl)oxy)methyl)-2,2-difluorocyclopropyl)methoxy)-6,8-difluoroquinazolin-4-yl)-1-methyl-3,8-diazabicyclo[3.2.1]octane-8-carboxylate (Int-90c)

Tert-Butyl (1S,5R)-3-(2-(((R)-2,2-difluoro-1-(hydroxymethyl)cyclopropyl)methoxy)-6,8-difluoroquinazolin-4-yl)-1-methyl-3,8-diazabicyclo[3.2.1]octane-8-carboxylate (Int-90b, 612 mg, 1.16 mmol) was added into a 20 mL vial. DCM (3.9 mL) was added into the reaction vessel. Imidazole (317 mg, 4.65 mmol) and tert-butylchlorodimethylsilane (350 mg, 2.33 mmol) were added into the reaction vessel. The resulting mixture was stirred for 2 h at 24° C. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 12-g silica gel column, eluting with hexanes initially, grading to 50% ethyl acetate in hexanes, linear gradient to afford tert-butyl (1S,5R)-3-(2-(((S)-1-(((tert-butyldimethylsilyl)oxy)methyl)-2,2-difluorocyclopropyl)methoxy)-6,8-difluoroquinazolin-4-yl)-1-methyl-3,8-diazabicyclo[3.2.1]octane-8-carboxylate (Int-90c). MS (ESI): m/z (M+H)⁺ 641.

### Step D: Tert-butyl (1S,5R)-3-(2-(((S)-1-(((tert-butyldimethylsilyl)oxy)methyl)-2,2-difluorocyclopropyl)methoxy)-7-(8-chloro-3-(methoxymethoxy)naphthalen-1-yl)-6,8-difluoroquinazolin-4-yl)-1-methyl-3,8-diazabicyclo[3.2.1]octane-8-carboxylate (Int-90d

In a glovebox, a solution of TMP₂Zn·MgCl₂—LiCl (687 μL, 0.103 mmol, 0.15 M in THF/toluene) was added in a 30 mL vial. In a separate vial, tert-butyl (1.5R)-3-(24(((S)-1-(((tert-butyldimethylsilyl)oxy)methyl)-2,2-difluorocyclopropyl)methoxy)-6,8-difluoroquinazolin-4-yl)-1-methyl-3,8-diazabicyclo[3.2.1]octane-8-carboxylate (Int-90c, 60 mg, 0.094 mmol) was dissolved in THF (1 mL). The Int-90c (starting material) solution was added dropwise into the zinc reagent. The starting material vessel was washed three times with THF (3×300 μLL) and the combined rinse was added dropwise into the reaction vessel. The resulting mixture was stirred for 1 h at 24° C. In a third vial, 1-bromo-8-chloro-3-(methoxymethoxy)naphthalene (Int-III4, 56.5 mg, 0.187 mmol) and CPhos-Pd-G4 (11.6 mg, 0.0141 mmol) were dissolved in dioxane (1 mL). This solution was added dropwise into the reaction vessel. The vessel containing the electrophile (Int-114) was rinsed with dioxane (3×300 μL). The combined rinses were added dropwise into the reaction vessel. The reaction vessel was removed from the glovebox and then heated at 100° C. for 2h. The product mixture was cooled down to room temperature and diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 24-g silica gel column, eluting with hexanes initially, grading to 50% ethyl acetate in hexanes, linear gradient to afford tert-butyl (1S,5R)-3-(2-(((S)-1-(((Iert-butyldimethylsilyl)oxy)methyl)-2,2-difluorocyclopropyl)methoxy)-7-(8-chloro-3-(methoxymethoxy)naphthalen-1-yl)-6,8-difluoroquinazolin-4-yl)-1-methyl-3,8-diazabicyclo[3.2.1]octane-8-carboxylate (Int-90d). MS (ESI): m/z (M+H)⁺ 861.

### Step E: Tert-butyl (1S,5R)-3-(24((S)-1-(((tert-butyldimethylsilyl)oxy)methyl)-2,2-difluorocyclopropyl)methoxy)-6,8-difluoro-7-(3-(methoxymethoxy)-8-((triisopropylsilyl)ethynyl)naphthalen-1-yl)quinazolin-4-yl)-1-methyl-3,8-diazabicyclo[3.2.1]octane-8-carboxylate (Int-90e)

Tert-butyl (1S,5R)-3-(2-(((S)-1-(((tert-butyldimethylsilyl)oxy)methyl)-2,2-difluorocyclopropyl)methoxy)-7-(8-chloro-3-(methoxymethoxy)naphthalen-1-yl)-6,8-difluoroquinazolin-4-yl)-1-methyl-3,8-diazabicyclo[3.2.1]octane-8-carboxylate (Int-90d) (81 mg, 0.094 mmol), ethynyltriisopropylsilane (211 μL, 0.940 mmol), Xphos-Pd-G3 (39.8 mg, 0.0470 mmol), and N-cyclohexyl-N-methyl)cyclohexanamine (92 mg, 0.47 mmol) were added into a 20 mL vial. The reaction vessel was evacuated and backfilled with a balloon of nitrogen three times. DMF (1.9 mL) was added into the reaction vessel. The resulting mixture was heated at 80° C. for 16 h. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 12-g amino-functionalized silica gel column (Sigma-Aldrich: 79297-1KG), eluting with hexanes initially, grading to 50% ethyl acetate in hexanes, linear gradient to afford the desired product tert-butyl (1S,5R)-3-(2-(((S)-1-(((tert-butyldimethylsilyl)oxy)methyl)-2,2-difluorocyclopropyl)methoxy)-6,8-difluoro-7-(3-(methoxymethoxy)-8-((triisopropylsilyl)ethynyl)naphthalen-1-yl)quinazolin-4-yl)-1-methyl-3,8-diazabicyclo[3.2.1]octane-8-carboxylate (Int-90e). MS (ESI): m/z (M+H)⁺ 1007.

### Step F: Teri-butyl (1S,5R)-3-(2-(((R)-2,2-difluoro-1-(hydroxymethyl)cyclopropyl)methoxy)-7-(8-ethynyl-3-(methoxymethoxy)naphthalen-1-yl)-6,8-difluoroquinazolin-4-yl)-1-methyl-3,8-diazabicyclo[3.2.1]octane-8-carboxylate (Int-90f-1)

Tert-butyl (1S,5R)-3-(2-(((S)-1-(((tert-butyldimethylsilyl)oxy)methyl)-2,2-difluorocyclopropyl)methoxy)-6,8-difluoro-7-(3-(methoxymethoxy)-8-((triisopropylsilyl)ethynyl)naphthalen-1-yl)quinazolin-4-yl)-1-methyl-3,8-diazabicyclo[3.2.1]octane-8-carboxylate (Int-90e) (45 mg, 0.045 mmol) was added in a 20 mL vial. The reaction vessel was evacuated and backfilled with a balloon of nitrogen three times. THF (893 μL) was added into the reaction vessel. A 1 M THF solution of tetrabutylammonium fluoride (112 μL, 0.112 mmol) was added dropwise. The resulting mixture was stirred for 30 min at 24° C. The product mixture was diluted with ethyl acetate (100 mL). The diluted product mixture was washed three times with saturated ammonium chloride aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 100% ethyl acetate/ethanol (3:1, v/v), linear gradient to afford the mixture of atropisomers. The material was resolved by SFC (Column M; Conditions: 30% IPA with 0.1% NH₄OH) to afford peak 1, the first-eluting isomer, as tert-butyl (1S,5R)-3-(2-(((R)-2,2-difluoro-1-(hydroxymethyl)cyclopropyl)methoxy)-7-(8-ethynyl-3-(methoxymethoxy)naphthalen-1-yl)-6,8-difluoroquinazolin-4-yl)-1-methyl-3,8-diazabicyclo[3.2.1]octane-8-carboxylate (Int-90f-1). MS (ESI): m/z (M+H)⁺ 737.

### Step G: 4-(2-(((R)-2,2-difluoro-1-(hydroxymethyl)cyclopropyl)methoxy)-6,8-difluoro-4-((1S,5R)-1-methyl-3,8-diazabicyclo[3.2.1]octan-3-yl)quinazolin-7-yl)-5-ethynylnaphthalen-2-ol (Ex. 90)

Tert-Butyl (S, 5R)-3-(2-(((R)-2,2-difluoro-1-(hydroxymethyl)cyclopropyl)methoxy)-7-(8-ethynyl-3-(methoxymethoxy)naphthalen-1-yl)-6,8-difluoroquinazolin-4-yl)-1-methyl-3,8-diazabicyclo[3.2.1]octane-8-carboxylate (Int-90f-1) (6.0 mg, 8.1 μmol) was added in an 8 mL vial. The reaction vessel was evacuated and backfilled with a balloon of nitrogen three times. 2-Propanol (181 uL) was added into the reaction vessel to dissolve the starting material. Then a 4 N solution of HCl in dioxane (90 μL) was added dropwise. The resulting mixture was heated at 45° C. for 1 h. The product mixture was concentrated to dryness. The residue obtained was purified via preparatory reverse phase HPLC (MeCN/water with 0.1% TFA modifier) to yield 4-(2-(((R)-2,2-difluoro-1-(hydroxymethyl)cyclopropyl)methoxy)-6,8-difluoro-4-((1S,5R)-1-methyl-3,8-diazabicyclo[3.2.1]octan-3-yl)quinazolin-7-yl)-5-ethynylnaphthalen-2-ol (Ex. 90). MS (ESI): m/z (M+H)⁺ 593. ¹H NMR (500 MHz, DMSO-d₆) δ 10.23 (s, 1H), 9.10 (s, 2H), 7.92 (d, J=8.1 Hz, 1H), 7.65 (d, J=9.9 Hz, 1H), 7.51-7.47 (m, 1H), 7.47-7.42 (m, 1H), 7.38 (d, J=2.6 Hz, 1H), 7.09 (d, J=2.6 Hz, 1H), 4.61 (d, J=11.2 Hz, 1H), 4.47-4.33 (m, 2H), 4.30 (d, J=13.6 Hz, 1H), 4.22 (s, 1H), 3.75-3.57 (m, 4H), 3.57-3.48 (m, 1H), 2.20 (s, 1H), 2.13-1.97 (m, 2H), 1.73 (d, J=27.0 Hz, 2H), 1.57 (s, 1H), 1.52 (s, 3H).

### Ex 205: (1S,5R)-3-(2-(((R)-2,2-difluoro-1-(((2-methoxyethyl)amino)methyl) cyclopropyl)methoxy)-68-difluoro-7-(6-methyl-5-(trifluoromethyl)-1H-indazol-4-yl)quinazolin-4-yl)-1-methyl-38-diazabicyclo[3.2.1]octan-8-ium 2,2,2-trifluoroacetate

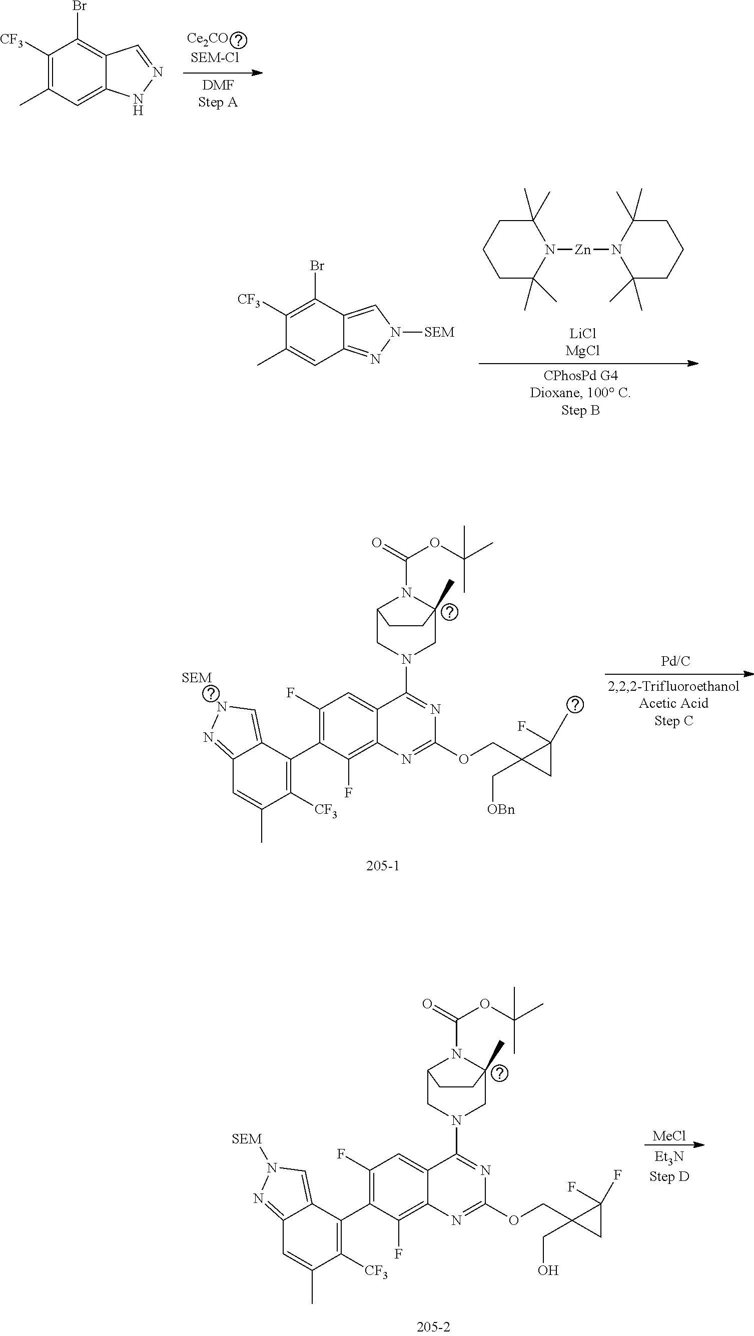

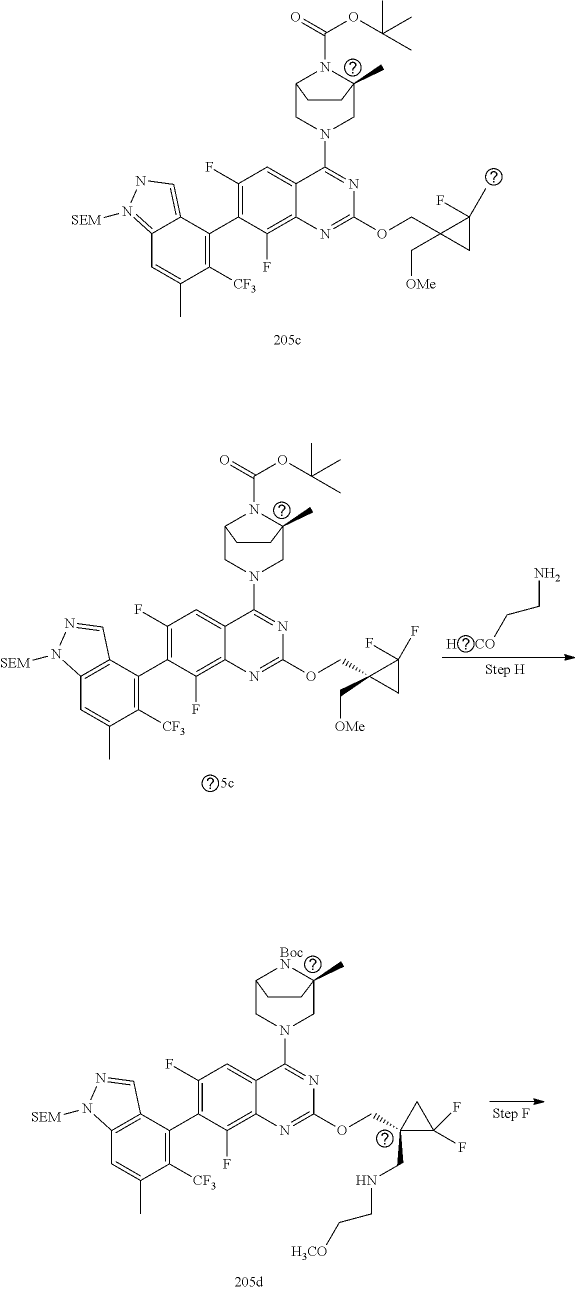

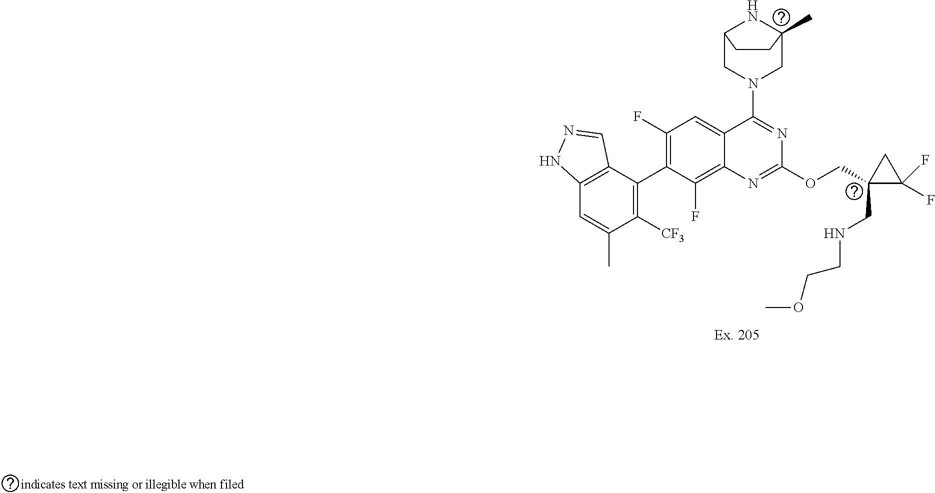 [SMILES: COCCNC[C@@]1(COc2nc(N3CC4CC[C@@](C)(C3)N4)c3cc(F)c(-c4c(C(F)(F)F)c(C)cc5[nH]ncc45)c(F)c3n2)CC1(F)F]

To a flask containing 4-bromo-6-methyl-5-(trifluoromethyl-1H-indazole (Int-HHH4) (3.3 g, 12 mmol) in DMF (59 mL) was added 2-(trimethylsih)ethoxymethyl chloride (3.8 ML,. 21 mmol) followed by cesium carbonate (9.7 g, 30 mmol). The mixture was allowed to stir for 18 hours at room temperature. After 18 hours, the mixture was then diluted with diethyl ether and water. The water layer was separated and re-extracted with diethyl ether. The combined organic layers were washed over magnesium sulfate, filtered and concentrated under reduced pressure. The resulting residue was purified by silica gel column chromatography (0-10% ethyl acetate gradient in hexanes) to afford 4-bromo-6-methyl-5-(trifluoromethyl)-2-((2-(trimethylsilyl)ethoxy)methyl)-2H-indazole. MS (ESI) m % z 409, 411 [M+H, M+2H]⁺.

### Step B: Tert-butyl 1(S-3424-((benzyloxy)methyl)-2,2-difluorocyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int-205-1)

A sealed vial was flushed with argon for 5 minutes. (TMP)₂Zn(MgCl₂LiCl) (0.15 M in toluene, 6.0 mL, 0.89 mmol) was added to the vial followed by a mixture of tert-butyl (1S,5R)-3-(2-(((R)₁-((benzyloxy)methyl)-2,2-difluorocyclopropyl)methoxy)-6,8-difluoroquinazolin-4-yl)-1-methyl-3,8-diazabicyclo[3.2.1]octane-8-carboxylate (Int-90a) (0.50 g, 0.81 mmol) in THF (4.0 mL). The mixture was allowed to stir for 45 minutes. After 45 minutes a vial was charged with 4-bromo-6-methyl-5-(trifluoromethyl)-2-((2-(trimethylsilyl)ethoxy)methyl)-2H-indazole (0.66 g, 1.6 mmol) and CPhos Pd G4 (0.10 g, 0.12 mmol). Dioxane (4.0 mL) was added. The resulting mixture was added to the first vial and the vial was placed in a reaction block that had been pre-heated to 100° C. After 90 minutes the mixture was allowed to cool to room temperature, quenched with saturated aqueous ammonium chloride and then successively extracted with ethyl acetate (3×). The combined organic layers were dried over anhydrous MgSO₄, filtered, and concentrated under reduced pressure. The crude residue was purified by silica gel chromatography (0-50% ethyl acetate gradient in hexanes) to afford tert-butyl 1(S)-3-(2-((1-(benzyloxy)methyl)-2,2-difluorocyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate. MS (ESI) m/z 945 [M+H, M+2H]⁺.

### Step C: Tert-butyl 1(S)-3-(2-((2,2-difluoro-1-(hydroxymethyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int-205-2)

To a flask containing tert-butyl 1(S)-3-(2-((1-((benzyloxy)methyl)-2,2-difluorocyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int 205-1) (0.39 g, 0.41 mmol) was added 2,2,2-trifluoroethanol (4.0 mL) and acetic acid (0.12 mL). The flask was flushed with nitrogen and then Pd/C (44 mg, 0.041 mmol) was added. The sealed flask was then evacuated and backfilled with hydrogen. This was repeated three more times. The flask was stirred for 18 hours at room temperature. After stirring overnight at room temperature, more acetic acid (50 uL) and Pd/C (44 mg, 0.041 mmol) were added. The sealed flask was then evacuated and backfilled with hydrogen. This process was repeated three more times. The flask was stirred for 18 hours at room temperature. After 18 hours, the mixture was filtered through CELITE and the CELITE was washed with methanol. The filtrate was then concentrated under reduced pressure. The resulting oil was diluted with ethyl acetate and saturated sodium bicarbonate. The organic layer was separated and the water layer was re-extracted with ethyl acetate. The combined organic layers were washed with brine, dried over magnesium sulfate, filtered and concentrated under reduced pressure. The resulting mixture was purified by silica gel column chromatography (0-20% ethyl acetate in hexanes) to afford tert-butyl 1(S)-3-(2-((2,2-difluoro-1-(hydroxymethyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate as a mixture of atropisomers. The atropisomers were separated by SFC (IC. 21×250, outlet pressure (bar): 100, flow rate (mL/min): 70 mL/min, modifier: 25% MeOH w/0.1% NH₄OH) to afford the separated isomers of tert-butyl 1(S)-3-(2-((2,2-difluoro-1-(hydroxymethyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate as Peak 1 (Int 205-2_P1) and tert-butyl 1(S)-3-(2-((2,2-difluoro-1-(hydroxymethyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate as Peak 2 (Int 205-2_P2). The two peaks were carried forward separately. Characterization data for Peak 1 from SFC: MS (EST) m/z 855 [M+H]⁺.

### Step D: Tert-butyl 1(S)-3-(2-(2,2-difluoro-1-(((methylsulfonyl)oxy)methyl) cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int-205c)

A flask containing tert-butyl 1(S)-3-(2-((2,2-difluoro-1-(hydroxymethyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int-205-2_P1) (Peak 1, Step C) (0.04 g, 0.047 mmol), dichloromethane (1.0 mL) and triethylamine (0.026 mL, 0.19 mmol) was cooled to 0° C. Methanesulfonyl chloride (7.3 μL, 0.094 mmol) was added. The ice bath was removed and the mixture was then stirred at room temperature for 1 hour. The mixture was then diluted with water (5.0 mL) and extracted with DCM (3×5.0 mL). The combined organic layers were dried over anhydrous MgSO₄, filtered, and concentrated under reduced pressure to provide tert-butyl 1(S)-3-(2-((2,2-difluoro-1-(((methylsulfonyl)oxy)methyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate that was taken on to the next step without further purification. MS (ESI) m/z 933 [M+H]⁺. (Int -205c) Tert-butyl 1(S)-3-(2-((2,2-difluoro-1-(((methylsulfonyl)oxy)methyl)cyclopropyl)methoxy)-6,8-difluoro-7-(6-methyl-5-(trifluoromethyl)-2-((2-(trimethylsilyl)ethoxy)methyl)-2H-indazol-4-yl)quinazolin-4-yl)-1-methyl-3,8-diazabicyclo[3.2.1]octane-8-carboxylate (Int-205c) was used as a common intermediate for synthesis of several compounds.

### STEP E: Tert-butyl 1(S)-3-(2-((2,2-difluoro-1-(((2-methoxyethyl)amino)methyl) cyclopropyl)methoxy)-6,8-difluoro-7-(6-methyl-5-(trifluoromethyl)-1-((2-(trimethylsilyl)ethoxy) methyl)-1H-indazol-4-yl)quinazolin-4-yl)-1-methyl-3,8-diazabicyclo[3.2.1]octane-8-carboxylate—PKI (Int-205d)

To a flask containing tert-butyl 1(S)-3-(2-((2,2-difluoro-1-(((methylsulfonyl)oxy)methyl)cyclopropyl)methoxy)-6,8-difluoro-7-(6-methyl-5-(trifluoromethyl)-1-((2-(trimethylsilyl)ethoxy)methyl)-1H-indazol-4-yl)quinazolin-4-yl)-1-methyl-3,8-diazabicyclo[3.2.1]octane-8-carboxylate; (30 mg, 0.032 mmol) was added THF (322 μl) followed by 2-methoxyethan-1-amine (12.08 mg, 0.161 mmol) and then K₂CO₃ (8.89 mg, 0.064 mmol). The mixture was heated to 75° C. overnight. Reaction was monitored using LCMS, Additional 2-methoxyethan-1-amine (36.2 mg. 0.482 mmol) was added and the mixture was then heated to 90° C. overnight. The reaction was cooled to rt, the mixture was diluted with ethyl acetate and water. The organic layer was separated, dried over magnesium sulfate, filtered and concentrated under reduced pressure. The resulting residue containing tert-butyl 1(S)-3-(2-((2,2-difluoro-1-(((2-methoxyethyl)amino)methyl) cyclopropyl)methoxy)-6,8-difluoro-7-(6-methyl-5-(trifluoromethyl)-1-((2-(trimethylsilyl)ethoxy) methyl)-1H-indazol-4-yl)quinazolin-4-yl)-1-methyl-3,8-diazabicyclo[3.2.1]octane-8-carboxylate was used in the next reaction without further purification.

### STEP F: (1S,5R)-3-(2-(((R)-2,2-difluoro-1-(((2-methoxyethyl)amino) methyl)cyclopropyl)methoxy)-6,8-difluoro-7-(6-methyl-5-(trifluoromethyl)-1H-indazol-4-yl)quinazolin-4-yl)-1-methyl-3,8-diazabicyclo[3.2.1]octan-8-ium 2,2,2-trifluoroacetate (Ex.205)

To a flask containing tert-butyl 1(S)-3-(2-((2,2-difluoro-1-(((2-methoxy ethyl)amino)methyl)cyclopropyl)methoxy)-6,8-difluoro-7-(6-methyl-5-(trifluoromethyl)-1-((2-(trimethylsilyl)ethoxy)methyl)-1H-indazol-4-yl)quinazolin-4-yl)-1-methyl-3,8-diazabicyclo[3.2.1]octane-8-carboxylate (Int-205d) (29 mg, 0.032 mmol) was added 2-Propanol (1 ml) and then HCl in Dioxane (0.5 mL, 2.000 mmol). The mixture was heated to 50° C. for 2 hours. Reaction was monitored using LCMS, The mixture was allowed to cool to room temperature and then diluted with ethyl acetate and saturated sodium bicarbonate. The organic layer was separated, dried over magnesium sulfate, filtered and concentrated under reduced pressure. The sample purified on a reverse phase HPLC using gradient elution with 0-100% ACN/water using 0.1% TFA as a modifier and then lyophilized to afford (1S,5R)-3-(2-(((R)-2,2-difluoro-1-(((2-methoxyethyl)amino)methyl)cyclopropyl)methoxy)-6,8-difluoro-7-(6-methyl-5-(trifluoromethyl)- 1H-indazol-4-yl)quinazolin-4-yl)-1-methyl-3,8-diazabicyclo[3.2.1]octan-8-ium 2,2,2-trifluoroacetate.

The compounds in the following table were synthesized following a similar procedure as Ex: 205 following STEP E and STEP F using commercially available appropriately protected amines/amino alcohols (Ex.213 to Ex.223).

| | | | | [M + H]⁺ | |
|---|---|---|---|---|---|
| Ex. | Starting Material | Structure | Compound Name | Found | SFC Purification Conditions |
| Ex. 206 | Negishi with CPhos Pd G3 50º C., 12 hr | 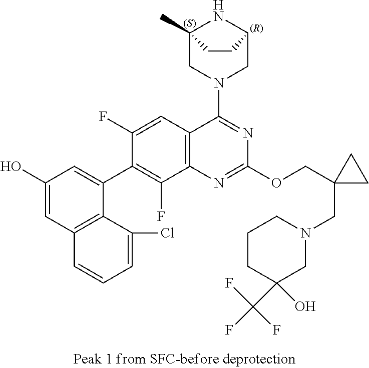 | rac-1-((1-(((7-(8-chloro-3-hydroxynaphthalen-1-yl)- 6,8-difluoro-4-((1S,5R)-1-methyl-3,8-diazabicyclo- [3.2.1]octan-3-yl)quinazolin-2-yl)oxy)methyl)cyclo- propyl)methyl)-3-(trifluoromethyl)piperidin-3-ol | 718 | SFC before deprotection Column A; Condition: 0.1% NH3H2O ETOH |
| Ex. 207 | Negishi with CPhos Pd G3 50° C., 12 hr | 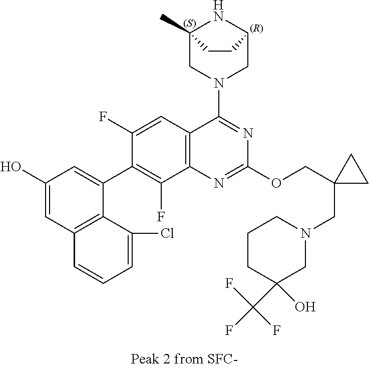 | rac-1-((1-(((7-(8-chloro-3-hydroxynaphthalen-1-yl)- 6,8-difluoro-4-((1S,5R)-1-methyl-3,8-diazabicyclo- [3.2.1]octan-3-yl)quinazolin-2-yl)oxy)methyl)cyclo- propyl)methyl)-3-trifluoromethyl)piperidin-3-ol | 718 | SFC before deprotection Column A; Condition: 0.1% NH3H2O ETOH |
| Ex. 208 | Negishi with CPhos Pd G3 50° C., 12 hr | 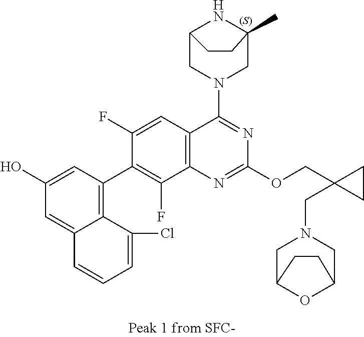 | 4-(2-((1-(((1R,5S)-8-oxa-3-zabicyclo[3.2.1]octan-3- yl)methyl)cyclopropyl)methoxy)-6,8-difluoro-4- ((1S,5R)-1-methyl-3,8-diazabicyclo[3.2.1]octan-3-l)- quinazolin-7-yl)-5-chloronaphthalen-2-ol | 662 | SFC before deprotection Column K; Condition: 0.1% NH3H2O ETOH |
| Ex. 209 | CPHOS Pd-G3, 50º C., 15 hr | 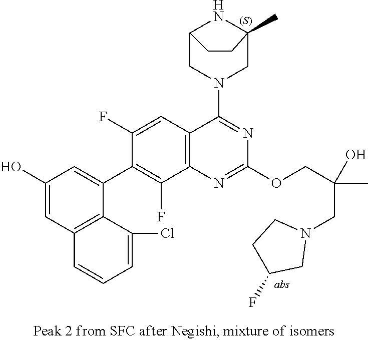 | 5-chloro-4-(6,8-difluoro-2-(3-((R)-3-fluoropyrrolidin- 1-yl)-2-hydroxy-2-methylpropoxy)-4-((1S,5R)-1- methyl-3,8-diazabicyclo[3.2.1]octan-3-yl)quinazolin- 7-yl)naphthalen-2-ol | 642 | Column: Chiralpak AD- 3150; Á4.6 mm I.D., 3 um Condition: A: CO2, B: iso- propanol (0.05% DEA) |
| Ex. 210 | CPHOS Pd-G3, 50° C., 15 hr | 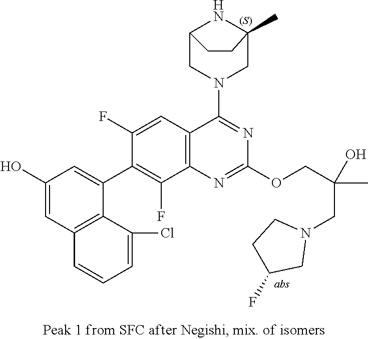 | 5-chloro-4-(6,8-difluoro-2-(3-((R)-3-fluoropyrrolidin- 1-yl)-2-hydroxy-2-methylpropoxy)-4-((1S,5R)-1- methyl-3,8-diazabicyclo[3.2.1]octan-3-yl)quinazolin- 7-yl)naphthalen-2-ol | 642 | Column: Chiralpak AD- 3150; Á4.6 mm I.D., 3 um Condition: A: CO2 B: iso- propanol (0.05% DEA) |
| Ex. 211a | 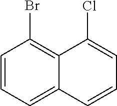 [SMILES: Clc1cccc2cccc(Br)c12] | 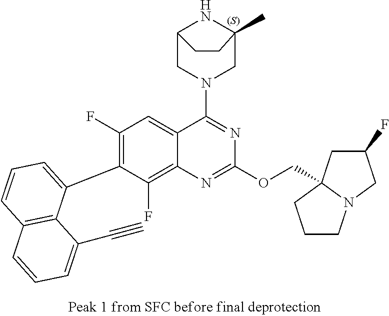 | 7-(8-ethynylnaphthalen-1-yl)-6,8-difluoro-2-(((2R, 7aS)-2-fluorotetrahydro-1H-pyrrolizin-7a(5H)-yl)- methoxy)-4-((1S,5R)-1-methyl-3,8-diazabicyclo- [3.2.1]octan-3-yl)quinazoline, 2TFA | 598 | Column L; condition: 40% MeOH w/0.1% NH4OH |
| Ex. 211b | 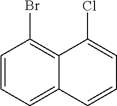 [SMILES: Clc1cccc2cccc(Br)c12] | 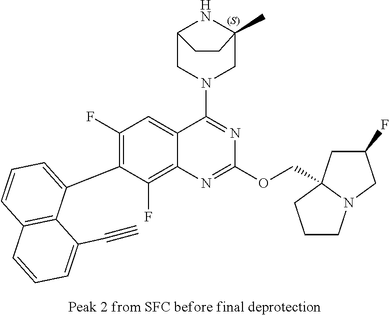 | 7-(8-ethynylnaphthalen-1-yl)-6,8-difluoro-2-(((2R, 7aS)-2-fluorotetrahydro-1H-pyrrolizin-7a(5H)-yl)- methoxy)-4-((1S,5R)-1-methyl-3,8-diazabicyclo- [3.2.1]octan-3-yl)quinazoline, 2TFA | 598 | Column L; condition: 40% MeOH w/0.1% NH4OH |
| Ex. 212 | 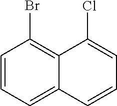 [SMILES: Clc1cccc2cccc(Br)c12] | 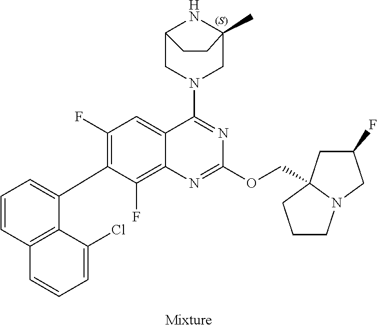 | 7-(8-chloronaphthalen-1-yl)-6,8-difluoro-2-(((2R, 7aS)-2-fluorotetrahydro-1H-pyrrolizin-7a(5H)-yl)- methoxy)-4-((1S,5R)-1-methyl-3,8-diazabicyclo- [3.2.1]octan-3-yl)quinazoline, 2TFA | 608 | -NA- |

| | | | [M + H]⁺ | |
|---|---|---|---|---|
| Ex. | Structure | Compound Name | Found | Purification conditions |
| Ex. 213 | 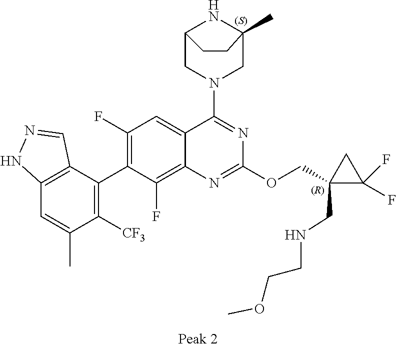 | (1S,5R)-3-(2-(((R)-2,2-difluoro-1-(((2-methoxyethyl)amino)methyl)- cyclopropylmethoxy-6,8-difluoro-7-(6-methyl-5-(trifluoromethyl)-1H- idazol-4-yl)quinazolin-4-yl)-1-methyl-3,8-diazabicyclo[3.2.1]octan-8- ium-2,2,2-trifluoroacetate | 682 | Column: IC, 21 × 250, modifier: 25% MeOH w/0.1% NH₄OH |
| Ex. 214a | 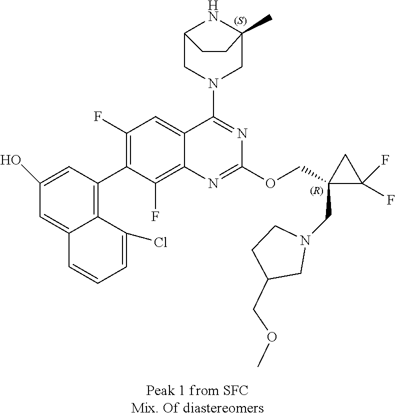 | 5-chloro-4-(2-(((1R)-2,2-difluoro-1-((3-(methoxymethyl)pyrrolidin-1-)- methylcyclopropylmethoxy-6,8-difluoro-4-((1S,5R)-1-methyl-3,8-diaza- bicyclo[3.2.1]octan-3-yl)-quinazolin-7-yl)naphthalen-2-ol | 700 | IC, 21 × 250 mm, 5 um; Modifier: 40% MeOH w/0.1% NH4OH |
| Ex. 214b | 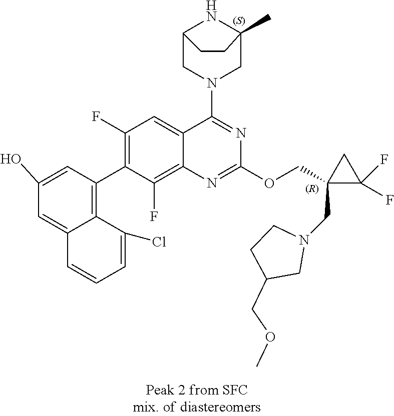 | 5-chloro-4-(2-(((1R)-2,2-difluoro-1-((3-(methoxymethyl)pyrrolidin-1-)- methyl)cyclopropyl)methoxy)-6,8-difluoro-4-((1S,5R)-1-methyl-3,8- diazabicyclo[3.2.1]octan-3-yl)quinazolin-7-yl)naphthalen-2-ol | 700 | IC, 21 × 250 mm, 5 um; Modifier: 40% MeOH w/0.1% NH4OH |
| Ex. 215a | 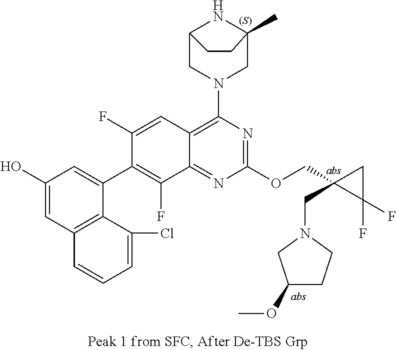 | 5-chloro-4-(2-(((1R)-2,2-difluoro-1-(((R)-3-methoxypyrrolidin-1-meth- yl)cyclopropyl)methoxy)-6,8-difluoro-4-((1S,5R)-1-methyl-3,8-diaza- bicyclo[3.2.1]octan-3-yl)quinazolin-7-yl)naphthalen-2-ol | 686 | |
| Ex. 215b | 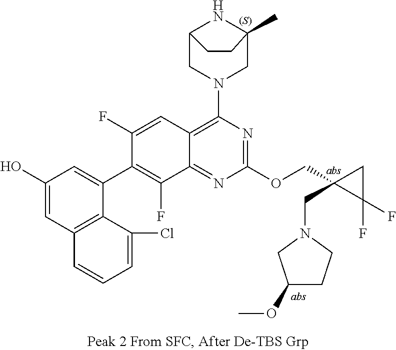 | 5-chloro-4-(2-(((R)-2,2difluoro-1-(((R)-3-methoxypyrrolidin-1-yl)meth- yl)cyclopropyl)methoxy-6,8-difluoro-4-((1S,5R)-1-methyl-3,8-diaza- bicyclo[3.2.1]octan-3-yl)quinazolin-7-yl)naphthalen-2-ol | 686 | |
| Ex. 216b | 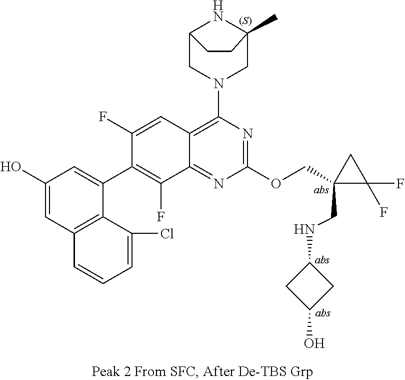 | 5-chloro-4-(2-(((R)-2,2difluoro-1-((((1S,3S)-3-hydroxycyclobutyl)- amino)methyl)cyclopropyl)methoxy)-6,8difluoro-4-((1S,5R)-1-methyl- 3,8-diazabicyclo[3.2.1]octan-3-yl)quinazolin-7-yl)naphthalen-2-ol | 672 | |
| Ex. 217b | 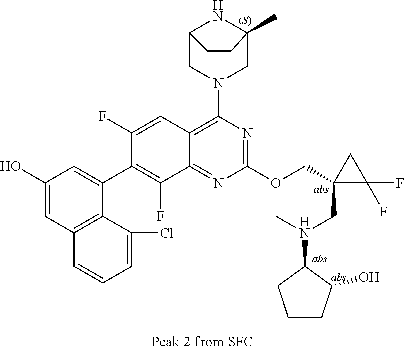 | 5-chloro-4-(2-(((R)-2,2-difluoro-1-((((1R,2R)-2-hydroxycyclopentyl)- (methyl)amino)methylcyclopropyl)methoxy)-6,8-difluoro-4-((1S,5R)- 1-methyl-3,8-diazabicyclo[3.2.1]octan-3-yl)quinazolin-7-yl)naphthalen- 2-ol | 700 | SFC separation at step C, before mesylation Column K; Condition 0.1% NH3H2O ETOH |
| Ex. 216a | 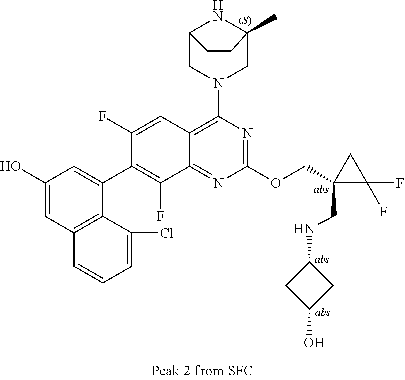 | tert-butyl (1S,5R)-3-(7-(8-chloro-3-(methoxymethoxy)naphthalen-1-yl)- 2-((((S)-2,2-difluoro-1-(((methylsulfonyl)oxy)methyl)cyclopropyl)meth- oxy)-6,8-difluoroquinazolin-4-yl)-1-methyl-3,8-diazabicyclo[3.2.1]- octane-8-carboxylate | 672 | SFC separation at step C, before mesylation Column K; Condition 0.1% NH3H2O ETOH |
| Ex. 218 | 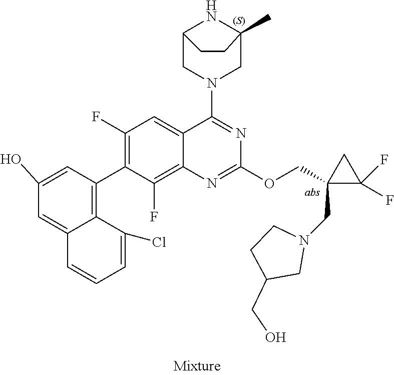 | 5-chloro-4-(2-(((1R)-2,2-difluoro-1-((3-(hydroxymethyl)pyrrolidin-1-)- methyl)cyclopropyl)methoxy)-6,8-difluoro-4-((1S,5R)-1-methyl-3,8- diazabicyclo[3.2.1]octan-3-yl)quinazolin-7-yl)naphthalen-2-ol | 686 | SFC separation at STEP C, Column: AS-H, 21 × 250 mm, 5 um Condition: 20% MeOH w/0.1% NH4OH |
| Ex. 217a | 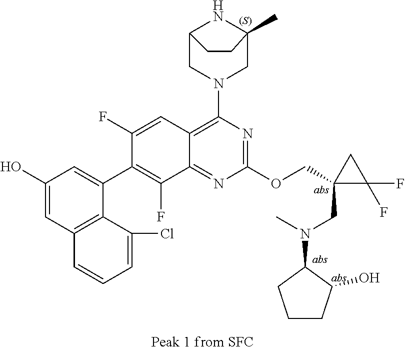 | 5-chloro-4-(2-(((R)-2,2-difluoro-1-((((1R,2R)-2-hydroxycyclopentyl)- (methyl)amino)methyl)cyclopropyl)methoxy)-6,8-difluoro-4-((1S,5R)- 1-methyl-3,8-diazabicyclo[3.2.1]octan-3-yl)quinazolin-7-yl)naphthalen- 2-ol | 700 | SFC separation at step C, before mesylation Column K; Condition 0.1% NH3H2O ETOH |
| Ex. 219a | 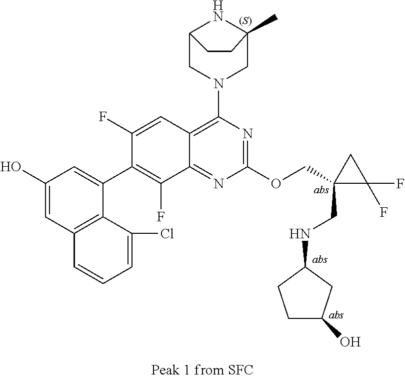 | 5-chloro-4-(2-(((R)-2,2-difluoro-1-((((1R,3S)-3-hydroxycyclopentyl)- amino)methyl)cyclopropyl)methoxy)-6,8-difluoro-4-((1S,5R)-1-methyl- 3,8-diazabicyclo[3.2.1]octan-3-yl)quinazolin-7-yl)naphthalen-2-ol | 686 | SFC separation at step C, before mesylation Column K; Condition 0.1% NH3H2O ETOH |
| Ex. 220 | 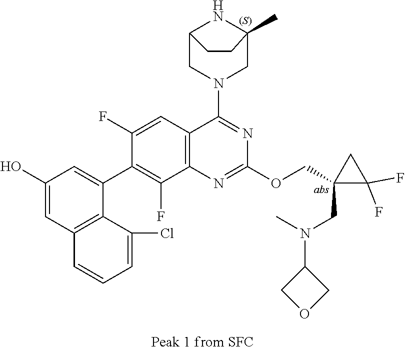 | 5-chloro-4-(2-(((R)-2,2-difluoro-1-((methyl(oxetan-3-l)amino)methyl)- cyclopropyl)methoxy)-6,8-difluoro-4-((1S,5R)-1-methyl-3,8-diaza- bicyclo[3.2.1]octan-3-yl)-quinazolin-7-yl)naphthalen-2-ol | 672 | SFC separation at step C, before mesylation Column K; Condition: 0.1% NH3H2O ETOH |
| Ex. 219b | 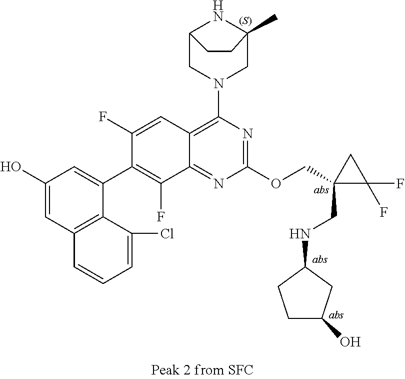 | 5-chloro-4-(2-(((R)-2,2-difluoro-1-((((1R,3S)-3-hydroxycyclopentyl)- amino)methyl)cyclopropyl)methoxy)-6,8-difluoro-4-((1S,5R)-1-methyl- 3,8-diazabicyclo[3.2.1]octan-3-yl)quinazolin-7-yl)naphthalen-2-ol | 686 | SFC separation at step C, before mesylation, Column K; Condition: 0.1% NH3H2O ETOH |
| Ex. 221 | 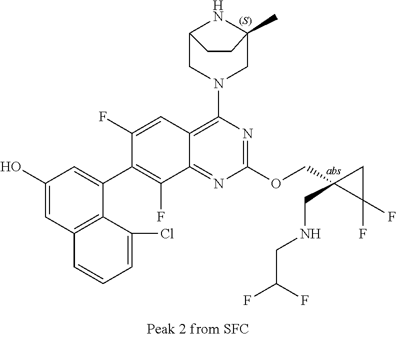 | 5-chloro-4-(2-(((R)-1-(((2,2-difluoroethyl)amino)methyl)-2,2-difluoro- cyclopropyl)methoxy-6,8-difluoro-4-((1S)-1-methyl-3,8-diazabicyclo- [3.2.1]octan-3-yl)quinazolin-7-yl)naphthalen-2-ol | 666 | SFC separation at step C before mesylation, Column K; Condition: 0.1% NH₃H₂O EtOH |
| Ex. 222 | 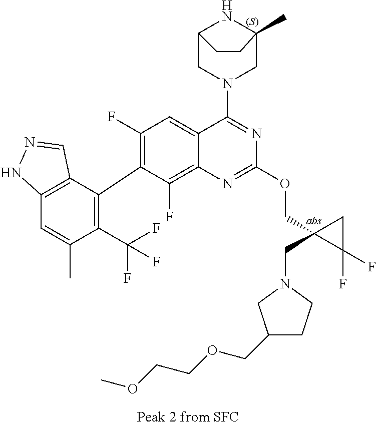 | (1S,5R)-3-(2-(((1R)-2,2-difluoro-1-((3-((2-methoxyethoxy)methyl)- pyrrolidin-1-yl)methyl)cyclopropyl)methoxy-6,8-difluoro-7-(6-methyl-5- (trifluoromethyl)-1H-indazol-4-yl)quinazolin-4-yl)-1-methyl-3.8-diaza- bicyclo[3.2.1]octan-8-ium 2,2,2-trifluoroacetate | 767 | SFC separation at step C before mesylation, Column K; Condition: 0.1% NH3H2O ETOH |
| Ex. 223 | 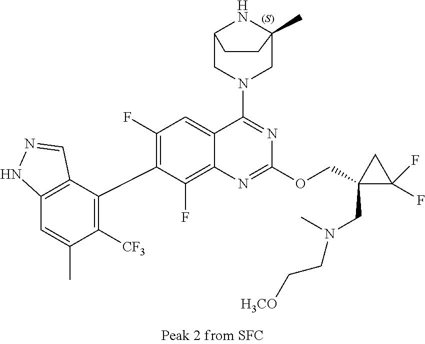 | (1S,5R)-3-(2-(((R)-2,2-difluoro-1-(((2-methoxyethyl)(methyl)amino)- methyl)cyclopropyl)methoxy)-6,8-difluoro-7-(6-methyl-5-(trifluorometh- yl)-1H-indazol-4-yl)quinazolin-4-yl)-1-methyl-3,8-diazabicyclo[3.2.1]- ocan-8-ium 2,2,2-trifluoroacetate | 696 | SFC separation at step C before mesylation, Column K; Condition: 0.1% NH3H2O ETOH |

### Example 91: 8-(6-chloro-8-fluoro-4-((1S,5R)-1-methyl-3,8-diazabicyclo[3.2.1]octan-3-yl)-2-((1-(morpholinomethyl)cyclopropyl)methoxy)quinazolin-7-yl)-5,7-difluoroquinolin-2-amine (Ex. 91a and Ex. 91b)

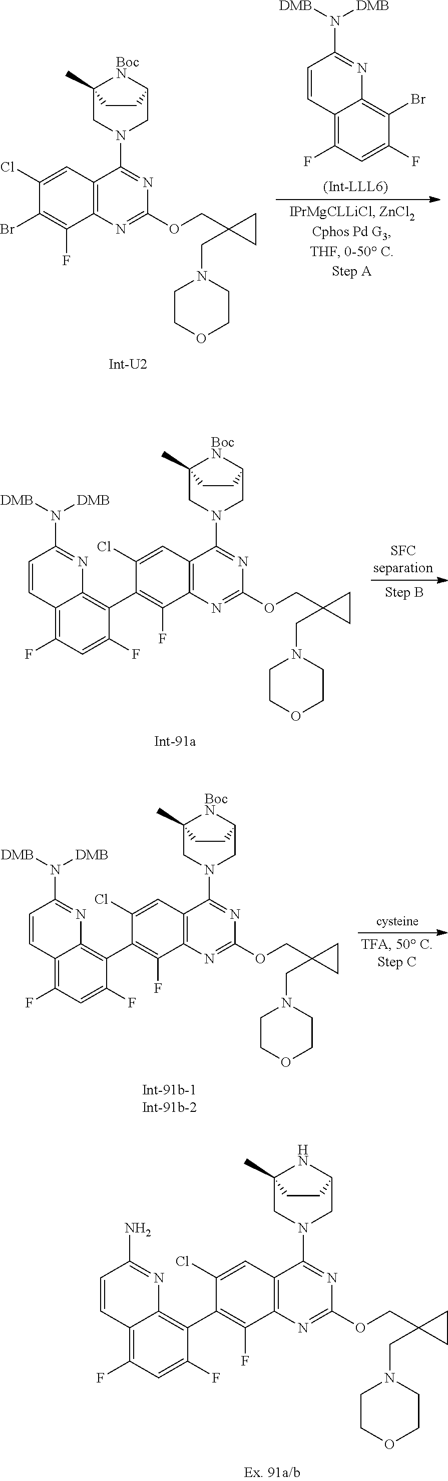

### Step A: Tert-butyl (1S,5R)-3-(7-(2-(bis(2,4-dimethylbenzyl)amino)-5,7-difluoroquinolin-8-yl-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-91a)

To a solution of tert-butyl (1S,5RP)-3-(7-bromo-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-U2) (50 mg, 0.076 mmol) in dry THF (0.5 mL) was added isopropylmagnesium chloride lithium chloride complex (0.123 mL. 0.160 mmol, 1.3 M in THF) dropwise at 0° C. under a N₂ atmosphere. After addition, the mixture was stirred at 0° C. for 0.5 h. Then to the above mixture was added zinc (II) chloride (0.122 mL, 0.122 mmol, IM in THF), and the resulting mixture was stirred at 0° C. for 10 min, then was stirred at 25° C. for 0.5 h. A solution of CPhos Pd G3 (12 mg, 0.015 mmol) and 8-bromo-N,N-bis(2,4-dimethoxybenzyl)-5,7-difluoroquinolin-2-amine (Int-LLL6) (85 mg, 0.15 mmol) in THF (0.5 mL) was added to the above solution dropwise in a glove box. After addition, the reaction was warmed to 50° C. and stirred for 16 h. The reaction mixture was purified directly by Prep-TLC (Pet, ether/EtOAc=1:1 v/v) to give tert-butyl (1S,5R)-3-(7-(2-(bis(2,4-dimethoxybenzyl)amino)-5,7-difluoroquinolin-8-yl)-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-91a). MS (ESI) [1/2M+H]⁺ m/z: 528.

### Step B: Tert-butyl (1S,5R)-3-(7-(2-(bis(2,4-dimethoxybenzyl)amino)-5,7-difluoroquinolin-8-yl)-6-chloro-8-fluoro-2-((1-(morpholinomethyl)cyclopropyl)methyl)quinazolin-4-yl)-1-methyl-3,8-diazabicyclo[3.2.1]octane-8-carboxylate (Int-91b-1 and Int-91b-2)

The mixture of diastereomers tert-butyl (1S, 5R)-3-(7-(2-(bis(2,4-dimethoxybenzyl)amino)-5,7-difluoroquinolin-8-yl)-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-91a) (73 mg, 0.069 mmol), was separated by preparative SFC (Column N, Condition: 0.1% NH₄OH in EtOH) to give tert-butyl (1S,5R)-3-(7-(2-(bis(2,4-dimethoxybenzyl)amino)-5,7-difluoroquinolin-8-yl)-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-91b-1) (the first eluting isomer) and tert-butyl (1S,5R)-3-(7-(2-(bis(2,4-dimethoxybenzyl)amino)-5,7-difluoroquinolin-8-yl)-6-chloro-8-fluoro-2-((1-(morpholinomethyl)cyclopropyl)methoxy)quinazolin-4-yl)-1-methyl-3,8-diazabicyclo[3.2.1]octane-8-carboxylate (Int-91b-2) (the second eluting isomer).

### Step C: 8-(6-chloro-8-fluoro-4-((1S,5R)-1-methyl-3,8-diazabicyclo[3.2.1]octan-3-yl)-2-((1-(morpholinomethyl)cyclopropyl)methoxy)quinazolin-7-yl)-5,7-difluoroquinolin-2-amine (Ex. 91a and Ex. 91b)

To a stirred solution of tert-butyl-1(S)-3-(7-(2-(bis(2,4-dimethoxybenzyl)amino)-5,7-difluoroquinolin-8-yl)-6-chloro-8-fluoro-2-((1-(morpholinomethyl)cyclopropyl)methoxy)quinazolin-4-yl)-1-methyl-3,8-diazabicyclo[3.2.1]octane-8-carboxylate (25 mg, 0.024 mmol) (Int-91b-1) in TFA (0.1 mL) was added L-cysteine (14.4 mg, 0.119 mmol) at 25° C. and the mixture was stirred at 50° C. for 0.5 h under a N₂ atmosphere. The solvent was evaporated under reduced pressure to give the crude product which was purified by reverse phase preparative HPLC (MeCN/water with 0.2% formic acid modifier) to give 8-(6-chloro-8-fluoro-4-((1S,5R)-1-methyl-3,8-diazabicyclo[3.2.1]octan-3-yl)-2-((1-(morpholinomethyl)cyclopropyl)methoxy)quinazolin-7-yl)-5,7-difluoroquinolin-2-amine (Ex. 91a). MS (ESI) [M+H]⁺ m:: 654. ¹H NMR (500 MHz, McOD) 5 8.16 (d, J=9.2 Hz, 1H), 7.96 (s, 1H), 7.01 (t. J=9.8 Hz, 1H), 6.87 (d, J=9.2 Hz, 1H), 4.57 (br d, J=13.7 Hz, 1H), 4.51-4.41 (m, 2H), 4.24 (br d, J=4.9 Hz, 1H), 3.92-3.81 (m, 5H), 3.81-3.70 (M. 2H), 3.29-2.95 (m, 6H), 2.36-2.21 (m, 2H), 2.20-2.06 (m, 1H), 1.97-1.82 (m, 1H), 1.62 (s, 3H), 0.91 (br s, 2H), 0.77 (br s. 2H).

8-(6-chloro-8-fluoro-4-((S, 5R)-1-methyl-3,8-diazabicyclo[3.2.1]octan-3-yl)-2-((1-(morpholinomethyl)cyclopropyl)methoxy)quinazolin-7-yl)-5,7-difluoroquinolin-2-amine (Ex. 91b) was prepared in the same manner as Ex. 91a, using Int-91b-2. MS (ESI) [M+H]⁺ m/z: 654. ¹H NMR (500 MHz, MeOD) δ 8.16 (d, J=9.2 Hz, 1H), 7.95 (br s, 1H), 7.00 (t, J=9.8 Hz, 1H), 6.86 (d, J=9.2 Hz, 1H), 4.71-4.37 (m, 4H), 4.22 (br s, 1H), 3.81 (br s, 6H), 3.13-2.74 (m, 6H), 2.27 (br s, 2H), 2.11 (br s, 1H), 1.93-1.81 (m, 11H), 1.61 (br s, 3H), 0.86 (br s, 2H), 0.70 (br s, 2H).

Compounds in the table below were synthesized according to a similar procedure as Ex. 91 above.

| | | | [M + | Purification |
|---|---|---|---|---|
| | | | H]⁺ | conditions |
| Ex. | Structure | Compound Name | Found | |
| Ex 92a | 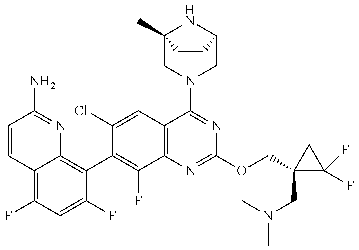 [SMILES: CN(C)C[C@@]1(COc2nc(N3C[C@H]4CC[C@@](C)(C3)N4)c3cc(Cl)c(-c4c(F)cc(F)c5ccc(N)nc45)c(F)c3n2)CC1(F)F] | 8-(6-chloro-2-(((R)-1-((dimethylamino)methyl)- 2,2-difluorocyclopropyl)methoxy)-8-fluoro-4- ((1S,5R)-1-methyl-3,8-diazabicyclo[3.2.1]octan- 3-yl)quinazolin-7-yl)-5,7-difluoroquinolin-2- amine | 648 | Column: Daicel Chiralpak IC (250 mm × 30 mm, 10 um); Condition: 0.1% NH₄OH in IPA |
| | Diastereomer 1 | | | |
| Ex 92b | 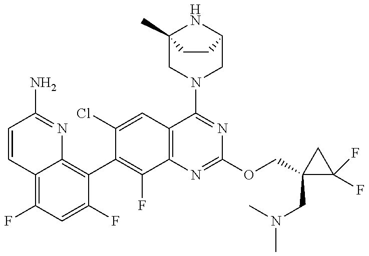 [SMILES: CN(C)C[C@@]1(COc2nc(N3C[C@H]4CC[C@@](C)(C3)N4)c3cc(Cl)c(-c4c(F)cc(F)c5ccc(N)nc45)c(F)c3n2)CC1(F)F] | 8-(6-chloro-2-(((R)-1-((dimethyalmino)methyl)- 2,2-difluorocyclopropyl)methoxy)-8-fluoro-4- ((1S,5R)-1-methyl-3,8-diazabicyclo[3.2.1]octan- 3-yl)quinazolin-7-yl)-5,7-difluoroquinolin-2- amine | 648 | |
| | Diastereomer 2 | | | |

### Example 93: 748-bromonaphthalen-1-yl)-24(1-(((R)-3-fluorovrrolidin-1-yl)methyl)cyclopropyl)methoxy)-4-((1S,5R)-1-methyl-3,8-diazabicyclo[3.2.1]octan-3-yl)-5,6,7,8-tetrahydropyrido[3.4-d]pyrimidine (Ex. 93)

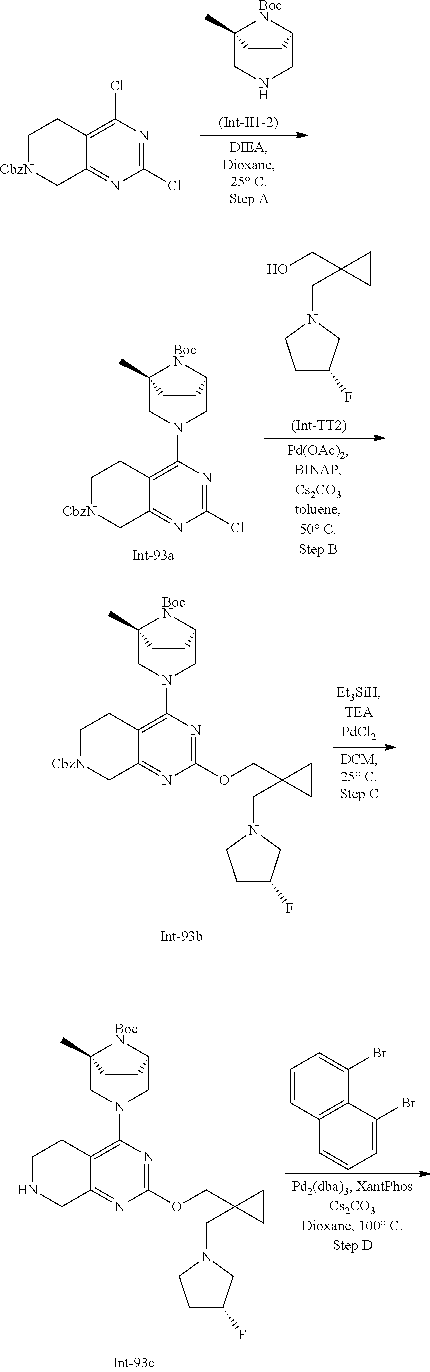

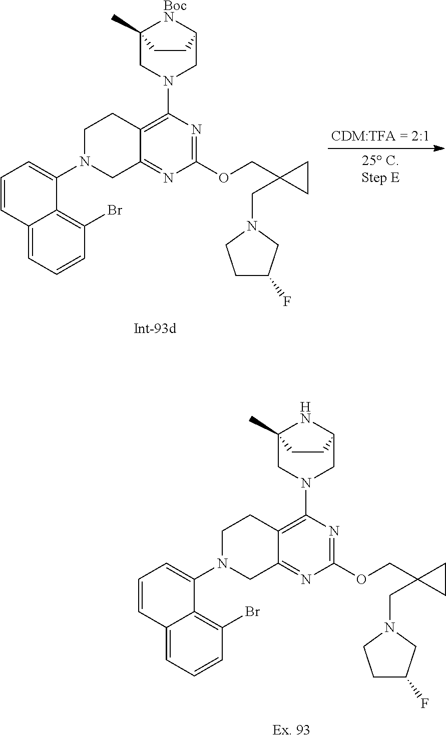 [SMILES: CC(C)(C)OC(=O)N1[C@@H]2CC[C@@]1(C)CN(c1nc(OCC3(CN4CC[C@@H](F)C4)CC3)nc3c1CCN(c1cccc4cccc(Br)c14)C3)C2 ; C[C@]12CC[C@H](CN(c3nc(OCC4(CN5CC[C@@H](F)C5)CC4)nc4c3CCN(c3cccc5cccc(Br)c35)C4)C1)N2]

### Step A: benzyl 4-((1S,5R)-84tert-butoxycarbonyl)-1-methyl-3,8-diazabicyclo[3.2.1]octan-3-yl)-2-chloro-5,8-dihydropyrido[3.4-d]pyrimidine-7(6H)-carboxylate (Int-93a)

To a solution of benzyl 2,4-dichloro-5,8-dihydropyrido[3.4-d]pyrimidine-7(6H)-carboxylate (1.00 g, 2.96 mmol) in dioxane (10 mL) were added tert-butyl (1S,5R)-1-methyl-3,8-diazabicyclo[3.2.1]octane-8-carboxylate (Int-III-2) (0.655 g, 2.89 mmol) and N,N-diisopropylethylamine (1.5 mL, 8.9 mmol) at 25° C. The reaction mixture was stirred at 25° C. for 12 h. The reaction mixture was quenched with H₂O (10 mL) and extracted with EtOAc (10 mL×3). The combined organic layers were dried over MgSO₄ and filtered. The filtrate was concentrated to give the residue which was purified by flash silica gel chromatography (Eluent of 0-15% EtOAc/Pet, ether gradient) to give benzyl 4-((1S,5R)-8-(tert-butoxycarbonyl)-1-methyl-3,8-diazabicyclo[3.2.1]octan-3-yl)-2-chloro-5,8-dihydropyrido[3.4-d]pyrimidine-7(6H)-carboxylate (Int-93a). MS (ESI): m/z (M+H)⁺ 528.

### Step B: Benzyl 4-((1S,5R)-8-(tert-butoxycarbonyl)-1-methyl-3,8-diazabicyclo[3.2.1]octan-3-yl)-2-((1-(((R)-3-fluoropyrrolidin-1-yl)methyl)cyclopropyl)methoxy)-5,8-dihydropyrido[3.4-d]pyrimidine-7(6H)-carboxylate (Int-93b)

A solution of benzyl 4-((1S,5R)-8-(tert-butoxycarbonyl)-1-methyl-3,8-diazabicyclo[3.2.1]octan-3-yl)-2-chloro-5,8-dihydropyrido[3.4-d]pyrimidine-7(6H)-carboxylate (250 mg, 0.473 mmol) (Int 93a), (R)-(1-((3-fluoropyrrolidin-1-yl)methyl)cyclopropyl)methanol (410 mg, 2.37 mmol), Cs₂CO₃ (463 mg, 1.42 mmol), BINAP (59.0 mg, 0.095 mmol) and palladium(II) acetate (10.6 mg, 0.0472 mmol) in toluene (5 mL) was set up in a glove box. The reaction mixture was stirred at 50° C. for 12 h. The reaction mixture was diluted with EtOAc (20 mL), dried over MgSO₄ and filtered. The filtrate was concentrated under reduced pressure to give the crude residue which was purified by flash silica gel chromatography (Eluent of 0-50% EtOAc/Pet, ether gradient) to give impure product. The product was further purified by Prep-TLC (SiO₂. (DCM/MeOH—NH₃=20:1)/(Pet, ether/EtOAc=5:1)=1:1, v/v) to give benzyl 4-((1S,5R)-8-(tert-butoxycarbonyl)-1-methyl-3,8-diazabicyclo[3.2.1]octan-3-yl)-2-((1-(((R)-3-fluoropyrrolidin-1-yl)methyl)cyclopropyl)methoxy)-5,8-dihydropyrido[3.4-d]pyrimidine-7(6H)-carboxylate (Int-93b). MS (ESI): m/z (M+H)⁺ 665.

### Step C: Tert-butyl (1S,5R)-3-(2-((1-(((R)-3-fluoropyrrolidin-1-yl)methyl)cyclopropyl)methoxy)-5.6,7,8-tetrahydropypyrido[3.4-d]pyrimidin-4-yl)-1-methyl-3,8-diazabicyclo[3.2.1]octane-8-carboxylate (Int-93c)

To a solution of benzyl 4-((1S,5R)-8-(tert-butoxycarbonyl)-1-methyl-3,8-diazabicyclo[3.2.1]octan-3-yl)-2-((1-(((R)-3-fluoropyrrolidin-1-yl)methyl)cyclopropyl)methoxy)-5,8-dihydropyrido[3.4-d]pyrimidine-7(6H)-carboxylate (Int-93b) (165 mg, 0.248 mmol), TEA (0.1 mL, 0.7 mmol) and triethylsilane (0.8 mL, 5 mmol) in DCM (7 mL) was added palladium(II) chloride (4.4 mg, 0.025 mmol). The reaction mixture was stirred at 25° C. for 12 h under N₂ protection. The reaction mixture was filtered and the filtered cake was washed with MeOH (10 mL). The filtrate was concentrated in vacuo to give the residue which was purified by flash silica gel chromatography (Eluent of 0-10% MeOH—NH₃/DCM), followed by Prep-TLC (SiO₂, DCM/MeOH—NH₃=15:1, v/v) to give tert-butyl (1S,5R)-3-(2-((1-(((R)-3-fluoropyrrolidin-1-yl)methyl)cyclopropyl)methoxy)-5,6,7,8-tetrahydropyrido[3.4-d]pyrimidin-4-yl)-1-methyl-3,8-diazabicyclo[3.2.1]octane-8-carboxylate (Int-93c). MS (ESI): m/z (M+H)⁺ 531.

### Step D: Tert-butyl (1S,5R)-3-(7-(8-bromonaphthalen-1-yl-2-((1-(((R)-3-fluoropyrrolidin-1-ylmethyl)cyclopropyl methoxy)-5.6,7,8-tetrahydropyrido[3.4-d]pyrimidin-4-yl)-1-methyl-3,8-diazabicyclo[3.2.1]octane-8-carboxylate (Int-93d)

A solution of tert-butyl (1S,5R)-3-(2-((1-(((R)-3-fluoropyrrolidin-1-yl)methyl)cyclopropyl)methoxy)-5,6,7,8-tetrahydropyrido[3.4-d]pyrimidin-4-yl)-1-methyl-3,8-diazabicyclo[3.2.1]octane-8-carboxylate (Int-93c) (40, mg, 0.075 mmol), 1.8-dibromonaphthalene (43.1 mg, 0.151 mmol), XantPhos (8.7 mg, 0.015 mmol), Cs₂CO₃ (73.7 mg, 0.226 mmol) and Pd₂(dba)₃ (6.90 mg, 7.54 μmol) in dioxane (1.0 mL) was set up in a glove box. The reaction mixture was stirred at 100° C. for 12 h. The reaction mixture was diluted with EtOAc (10 mL) and filtered. The filtrate was concentrated under reduced pressure to give the residue which was purified by Prep-TLC (SiO₂, Pet, ether/EtOAc=2:1, v/v) to give tert-butyl (1S,5R)-3-(7-(8-bromonaphthalen-1-yl)-24(1-(((R)-3-fluoropyrrolidin-1-yl)methyl)cyclopropyl)methoxy)-5,6,7,8-tetrahydropyrido[3.4-d]pyrimidin-4-yl)-1-methyl-3,8-diazabicyclo[3.2.1]octane-8-carboxylate (Int-93d). MS (ESI): m/z (M+H)⁺ 735, 737.

### Step E: 7-(8-bromonaphthalen-1-yl)-2-((1-(((R)-3-fluoropyrrolidin-1-VI)methyl)cyclopropyl)methoxy)-4-((1S,5R)-1-methyl-3,8-diazabicyclo[3.2.1]octan-3-yl)-5.6,7,8-tetrahydropyrido[3.4-d]pyrimidine (Ex

To a solution of tert-butyl (1S,5R)-3-(7-(8-bromonaphthalen-1-yl)-2-((1-(((R)-3-fluoropyrrolidin-1-yl)methyl)cyclopropyl)methoxy)-5,6,7,8-tetrahydropyrido[3.4-d]pyrimidin-4-yl)-1-methyl-3,8-diazabicyclo[3.2.1]octane-8-carboxylate (Int-93d) (42 mg, 0.057 mmol) in DCM (1.0 mL) was added TFA (0.5 mL). The reaction mixture was stirred at 25° C. for 10 min. The reaction was bubbled with N₂ to remove the solvent. The residue was diluted with EtOAc (5 mL) and basified by cooled (0° C.) NaHCO₃(aq.) (3 mL). The aqueous layer was extracted with EtOAc (5 mL×3). The combined organic layers were dried over MgSO₄ and filtered. The filtrate was concentrated under reduced pressure to give the residue which was purified by reverse phase Prep-HPLC (MeCN/water with 0.1% TFA modifier) to give 7-(8-bromonaphthalen-1-yl)-2-((1-(((R)-3-fluoropyrrolidin-1-yl)methyl)cyclopropyl)methoxy)-4-((1S,5R)-1-methyl-3,8-diazabicyclo[3.2.1]octan-3-yl)-5,6,7,8-tetrahydropyrido[3.4-d]pyrimidine (Ex. 93). MS (ESI): m/z (M+H)⁺ 635/637. ¹H NMR (400 MHz, McOD) 5 7.84 (d, J=8.07 Hz, 1H), 7.78 (d, J=7.34 Hz, 1H), 7.66 (d, J=7.83 Hz, 1H), 7.48 (t, J=7.83 Hz, 1H), 7.32 (d, J=7.09 Hz, 1H), 7.25 (t, J=7.83 Hz, 1H), 5.00-5.21 (m, 1H). 4.59 (br s, 2H), 4.13-4.32 (m, 4H), 3.61-3.74 (m, 1H), 3.50-3.59 (m, 3H), 3.12-3.22 (m, 2H), 2.83-2.97 (m, 2H), 2.63-2.75 (m, 1H), 2.55-2.62 (m, 2H), 2.42 (dd, J=6.60, 12.47 Hz, 2H), 1.84-2.12 (m, 4H), 1.68-1.82 (m, 1H), 1.49 (td, J=6.30, 12,35 Hz, 1H), 1.28 (s, 3H), 0.62 (s, 2H), 0.46 (s, 2H).

Examples in the table below were prepared via an analogous synthetic sequence as Ex. 93 above.

| | Starting | | | [M + H]⁺ |
|---|---|---|---|---|
| Ex. | Material | Structure | Compound Name | Found |
| Ex 94 | 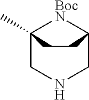 [SMILES: CC(C)(C)OC(=O)N1[C@H]2CC[C@]1(C)CNC2] Int II1-1 | 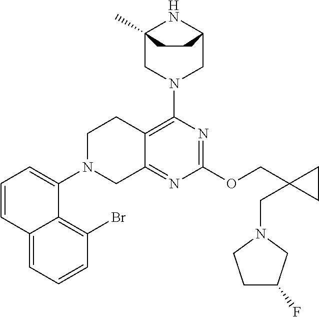 [SMILES: C[C@@]12CC[C@@H](CN(c3nc(OCC4(CN5CC[C@@H](F)C5)CC4)nc4c3CCN(c3cccc5cccc(Br)c35)C4)C1)N2] | 7-(8-bromonaphthalen-1-yl)-2-((1-(((R)-3-fluoropyrroli- din-1-yl)methyl)cyclopropyl)methoxy)-4-((1S,5R)-1- methyl-3,8-diazabicyclo[3.2.1]octan-3-yl)-5,6,7,8- tetrahydropyrido[3,4-d]pyrimidine | 635/637 |

### Example 95: 4-(3.8-diazabicyclo[3.2.1]octan-3-v)-7-(3-hydroxynaphthalen-1-yl)-2-((1-(morpholinomethy)cyclopropyl)methoxy)-5H-pyrano[4.3-d]pyrimidin-1-one

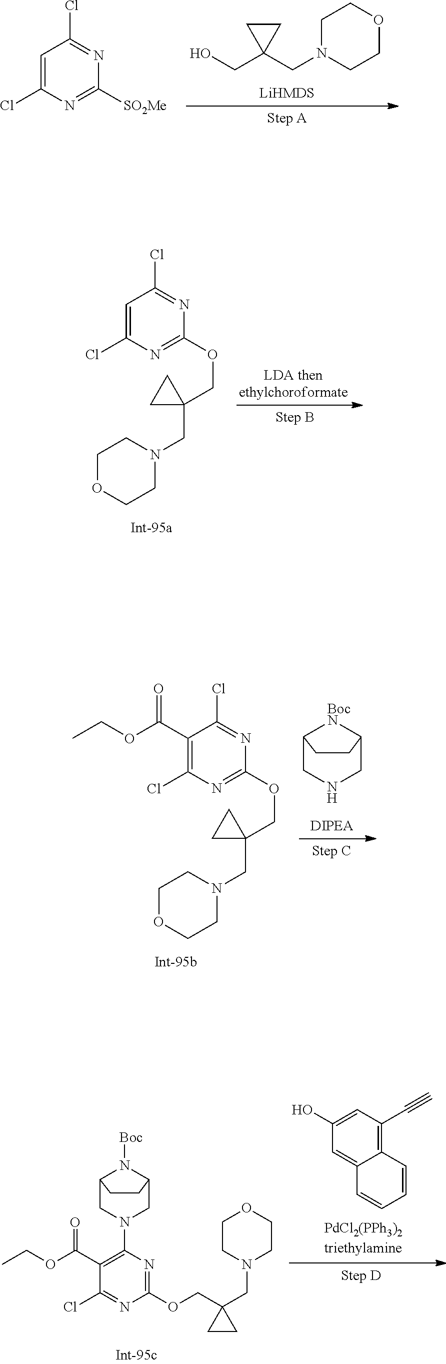

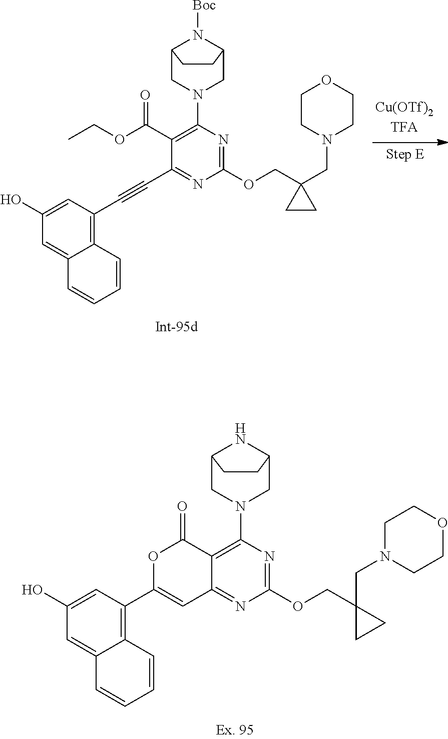 [SMILES: CCOC(=O)c1c(C#Cc2cc(O)cc3ccccc23)nc(OCC2(CN3CCOCC3)CC2)nc1N1CC2CCC(C1)N2C(=O)OC(C)(C)C ; O=c1oc(-c2cc(O)cc3ccccc23)cc2nc(OCC3(CN4CCOCC4)CC3)nc(N3CC4CCC(C3)N4)c12]

### Step A: 4-((1-(((4,6-dichlorooyrimidin-2-yl)oxy)methyl)cyclopropyl)methyl)morpholine (Int-95a)

A 100 mL round bottom flask equipped with a stirbar was charged with (1-(morpholinomethyl)cyclopropyl)methanol (11.3 g, 66.1 mmol). The flask was capped with a septum and placed under a nitrogen atmosphere and THF (75 mL) was added. The reaction mixture was cooled to −30° C. and LiHMDS (66 mL, 99 mmol) was added. This mixture was stirred for 30 minutes and then was added to a solution of 4,6-dichloro-2-(methylsulfonyl)pyrimidine (15 g, 66 mmol) in THF (75 mL) at −30° C. This mixture was stirred at this temperature for 1 h and then quenched with aqueous saturated ammonium chloride (200 mL) and extracted with ethyl acetate. The combined organic layers were dried over MgSO₄, filtered, and the filtrate was concentrated. The residue was purified by column chromatography on silica gel (0 to 30% ethyl acetate/hexanes) to afford 4-((1-(((4,6-dichloropyrimidin-2-yl)oxy)methyl)cyclopropyl)methyl)morpholine (Int-95a). MS (ESI) [M+H]⁺ m/z 320.

### Step B: ethyl 4,6-dichloro-2-((1-(morpholinomethyl)cyclopropyl)methoxy)pyrimidine-5-carboxylate (Int-95b)

A 250 mL round bottom flask equipped with a stirbar was charged with 4-((1-(((4,6-dichloropyrimidin-2-yl)oxy)methyl)cyclopropyl)methyl)morpholine (Int-95a) (10 g, 31 mmol). The flask was capped with a septum and the contents were placed under a nitrogen atmosphere. THF (100 mL) was added and the reaction mixture was cooled to −78° C. To the stirring solution was added a solution of LDA in (2 M in THF/heptane/ethylbenzene, 17.3 mL, 34.6 mmol) dropwise. The mixture was stirred at −78° C. for 15 minutes. To this mixture was added ethyl carbonochloridate (3.16 mL, 33.0 mmol) dropwise and the mixture was stirred for an additional 15 minutes at −78° C. and then warmed to ambient temperature. The reaction mixture was quenched by adding water and brine and the mixture was extracted with ethyl acetate. The combined organic layers were dried over MgSO₄, filtered, and the filtrate was concentrated to afford ethyl 4,6-dichloro-2-((1-(morpholinomethyl)cyclopropyl)methoxy)pyrimidine-5-carboxylate (Int-95b), which was used without further purification. MS (ESI) [M+H]⁺ m: 390, 392.

### Step C: Teri-butyl 3-(6-chloro-5-(ethoxycarbonyl)-2-((1-(morpholinomethyl)cyclopropyl)methyl)pyrimidin-4-yl)-3,8-diazabicyclo[3.2.1]octane-8-carboxylate (Int-95c)

A solution of ethyl 4,6-dichloro-2-((1-(morpholinomethyl)cyclopropyl)methoxy)pyrimidine-5-carboxylate (Int-95b) (12,3 g, 31.4 mmol) in DCM (123 mL) was cooled to −78° C. and N,N-diisopropylethylamine (8.23 mL, 47.1 mmol) was added. To this stirring mixture was added tert-butyl-3,8-diazabicyclo[3.2.1]octane-8-carboxylate (7.0 g, 33.0 mmol). The reaction mixture was warmed to ambient temperature and stirred for 1.5 h. The mixture was concentrated and the residue was purified by column chromatography on silica gel (0 to 20% [1:3 EtOH/EtOAc v/v]/hexanes) to afford tert-butyl 3-(6-chloro-5-(ethoxycarbonyl)-2-((1-(morpholinomethyl)cyclopropyl)methoxy)pyrimidin-4-yl)-3,8-diazabicyclo[3.2.1]octane-8-carboxylate (Int-95c). MS (ESI) [M+H]⁺ m/z 566, 568. Step D: Tert-butyl-1(S)-3-(5-(ethoxycarbonyl)-6-((3-hydroxynaphthalen-1-yl)ethynl)-2-((1-(morpholinomethyl)cyclopropyl)methoxy)pyrimidin-4-yl)-3,8-diazabicyclo[3.2.1]octane-8-carboxylate (Int-95d)

Tert-butyl-1(S)-3-(6-chloro-5-(ethoxycarbonyl)-2-((1-(morpholinomethyl)cyclopropyl)methoxy)pyrimidin-4-yl)-3,8-diazabicyclo[3.2.1]octane-8-carboxylate (Int-95c) (600 mg, 1.06 mmol), 4-ethynylnaphthalen-2-ol (357 mg, 2.12 mmol) and triethylamine (0.295 mL. 2.12 mmol) in acetonitrile (3.0 mL) were subsurface sparged with nitrogen for 3 minutes and treated with bis(triphenylphosphine)palladium(II) dichloride (149 mg, 0.212 mmol). The mixture was heated at 65° C. for 8 h, cooled to ambient temperature and concentrated. The residue was purified by column chromatography on silica gel (40 g; 0 to 100% EtOAc/hexanes; eluted at 100%) to afford tert-butyl-1(S)-3-(5-(ethoxycarbonyl)-6-((3-hydroxynaphthalen-1-yl)ethynyl)-2-((1-(morpholinomethyl)cyclopropyl)methoxy)pyrimidin-4-yl)-3,8-diazabicyclo[3.2.1]octane-8-carboxylate (Int-95d). MS (ESI) [M+H]⁺ m/z 698.

### Step E: 4-(3.8-diazabicyclo[3.2.1]octan-3-yl)-7-(3-hydroxynaphthalen-1-yl)-2-((1-(morpholinomethyl)cyclopropyl)methoxy)-5H-pyrano[4.3-d]pyrimidin-5-one (Ex. 95)

Tert-butyl 3-(5-(ethoxycarbonyl)-6-((3-hydroxynaphthalen-1-yl)ethynyl)-2-((1-(morpholinomethyl)cyclopropyl)methoxy)pyrimidin-4-yl)-3,8-diazabicyclo[3.2.1]octane-8-carboxylate (Int-95d) (100 mg, 0.143 mmol) was dissolved in TFA (1.0 mL) and treated with copper(II) trifluoromethanesulfonate (51.8 mg, 0.143 mmol). The mixture was heated at 40° C. for 6 h, cooled to rt and concentrated. The crude residue was dissolved in acetonitrile, filtered and the filtrate was purified by preparative RP-HPLC (MeCN/water with 0.1% TFA modifier) to afford 4-(3.8-diazabicyclo[3.2.1]octan-3-yl)-7-(3-hydroxynaphthalen-1-yl)-2-((m-(morpholinomethyl)cyclopropyl)methoxy)-5H-pyrano[4,3-d]pyrimidin-5-one (Ex. 95). MS (ESI) [M+H]⁺ m/z 570, 1H NMR (499 MHz, DMSO-d₆) δ 10.20 (s, 1H), 9.87 (s, 1H), 9.33 (s, 1H), 9.22 (s, 1H), 8.08 (d, J=8.6 Hz, 1H), 7.84 (d, J=8.3 Hz, 1H), 7.51 (t, J=7.6 Hz, 1H), 7.43-7.31 (m, 3H), 6.78 (s, 1H), 4.30 (s, 2H), 4.29-4.22 (m, 2H), 4.22-4.11 (m, 2H), 4.08-3.98 (m, 2H), 3.80-3.67 (m, 2H), 3.65-3.55 (m, 4H), 3.32-3.24 (m, 2H), 3.20-3.06 (m, 2H), 1.94 (s, 4H), 0.84 (d, J=26.6 Hz, 4H).

### Example 96: 2-(((R)-1-((dimethylamino)methyl)-2,2-difluorocyclopropylmethoxy)-7-(6-methyl-5-(trifluoromethyl)-1H-indazol-4-yl)-4-((1S, 5R)-1-methyl-3,8-diazabicyclo[3.2.1]octan-3-yl)-7,8-dihydro-5H-pyranol[4,3-d]pyrimidin-5-one (Ex. 96a and Ex. 96b)

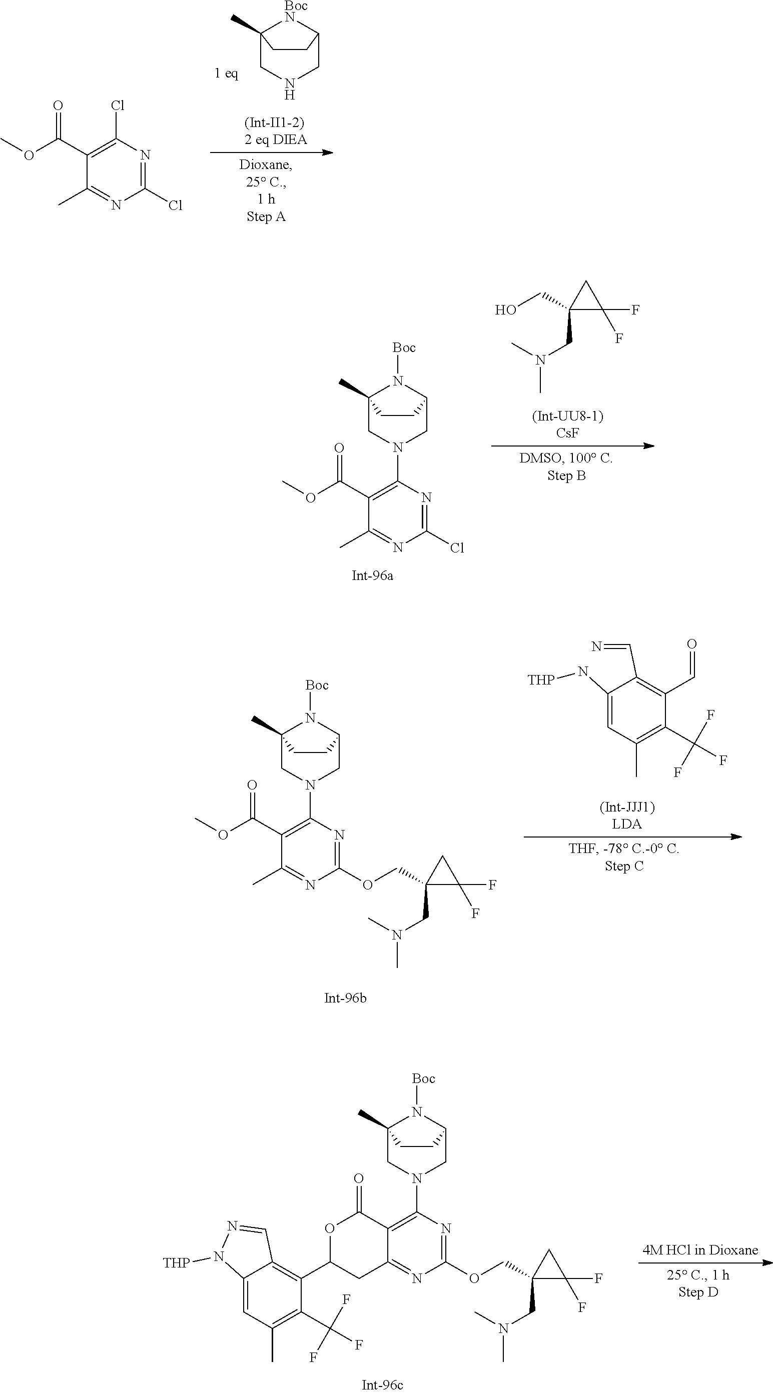

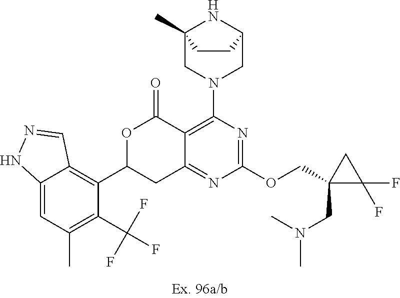 [SMILES: Cc1cc2[nH]ncc2c(C2Cc3nc(OC[C@]4(CN(C)C)CC4(F)F)nc(N4C[C@H]5CC[C@@](C)(C4)N5)c3C(=O)O2)c1C(F)(F)F]

### Step A: Teri-butyl (1S,5R)-3-(2-chloro-5-(methoxycarbonyl)-6-methylpyrimidin-4-yl-1-methyl-3,8-diazabicyclo[3.2.1]octane-8-carboxylate (Int-96a)

To a stirred solution of methyl 2,4-dichloro-6-methylpyrimidine-5-carboxylate (2 g, 9 mmol) and DIEA (3.16 mL, 18.1 mmol) in dioxane (50 mL) was added tert-butyl (1S,5R)-1-methyl-3,8-diazabicyclo[3.2.1]octane-8-carboxylate (Int-111-2) (2.05 g, 9.05 mmol) at 25° C. After the addition was finished, the reaction was stirred at 25° C. for 3 h. The mixture was concentrated and purified by flash silica gel chromatography (eluent of 0-36% Ethyl acetate/Petroleum ether gradient) to give tert-butyl (1S,5R)-3-(2-chloro-5-(methoxy carbonyl)-6-methylpyrimidin-4-yl)-1-methyl-3,8-diazabicyclo[3.2.1]octane-8-carboxylate (Int-96a). MS (ESI) [M+H]⁺ m z 411.

### Step B: Tert-butyl (,55R)-3-(2-(((R)-1-((dimethylamino)methyl)-2,2-difluorocyclopropyl)methoxyl)-5-(methoxy carbonyl)-6-methylpyrimidin-4-yl)-1-methyl-3,8-diazabicyclo[3.2.1]octane-8-carboxylate (Int-% b)

To a stirred solution of tert-butyl (1S,5R)-3-(2-chloro-5-(methoxycarbonyl)-6-methylpyrimidin-4-yl)-1-methyl-3,8-diazabicyclo[3.2.1]octane-8-carboxylate (Int-% a) (407 mg, 0.991 mmol) and (R)-(I-((dimethylamino)methyl)-2,2-difluorocyclopropyl)methanol (Int-UU8-1) (400 mg, 0.991 mmol) in DMSO (8 mL) was added CsF (602 mg, 3.96 mmol) at 25° C. under N₂. After the addition was finished, the reaction was stirred at 100° C. for 16 h under N₂. To the mixture was added water (20 mL), it was extracted with EtOAc (15 mL×3), the organic layers was washed with brine (20 mL), dried over Na₂SO₄ and concentrated. The residue was purified by flash silica gel chromatography (Eluent of 0-36% Ethyl acetate/Petroleum ether gradient) to give tert-butyl (1S,5R)-3-(2-(((R)-1-((dimethylamino)methyl)-2,2-difluorocyclopropyl)methoxy)-5-(methoxycarbonyl)-6-methylpyrimidin-4-yl)-1-methyl-3,8-diazabicyclo[3.2.1]octane-8-carboxylate (Int-96b). MS (ESI) [M+H]⁺ m,: 540.

### Step C: Tert-butyl (1S,5R)-3-(2-(((R)-1-((dimethylamino)methyl)-2,2-difluorocyclopropyl)methoxy)-7-(6-methyl-14tetrahydro-2H-pyran-2-yl)-5-(trifluoromethyl)-1H-indazol-4-yl)-5-oxo-7,8-dihydro-5H-pyrano[4,3-d]pyrimidin-4-yl)-1-methyl-3,8-diazabicyclo[3.2.1]octane-8-carboxylate (Int-96c)

To a stirred solution of tert-butyl (1S,5R)-3-(2-(((R)-1-((dimethylamino)methyl)-2,2-difluorocyclopropyl)methoxy)-5-(methoxycarbonyl)-6-methylpyrimidin-4-yl)-1-methyl-3,8-diazabicyclo[3.2.1]octane-8-carboxylate (Int-96b) (250 mg, 0.463 mmol) in THF (5 mL) was added LDA (0.6 mL, 0.6 mmol, 1 M in THF) at −78° C. under N₂, and the reaction was stirred at −78° C. for 0.5 h. Then 6-chloro-3,5-dimethyl-1-(tetrahydro-2H-pyran-2-yl)-1H-indazole-4-carbaldehyde (270 mg, 0.926 mmol) was added at −78° C. under N₂, and the reaction was stirred at 0° C. for 0.5 h under N₂. To the mixture was added saturated NH₄Cl aqueous (4 mL), and the mixture was extracted with EtOAc (4 mL×3). The combined organic layers were washed with brine (10 mL), dried over Na₂SO₄ and concentrated. The residue was purified by flash silica gel chromatography (Eluent of 0-30% Ethyl acetate/Petroleum ether gradient) to give tert-butyl (1S,5R)-3-(2-(((R)-1-((dimethylamino)methyl)-2,2-difluorocyclopropyl)methoxy)-7-(6-methyl-1-(tetrahydro-2H-pyran-2-yl)-5-(trifluoromethyl)-1H-indazol-4-yl)-5-oxo-7,8-dihydro-5H-pyrano[4,3-d]pyrimidin-4-yl)-1-methyl-3,8-diazabicyclo[3.2.1]octane-8-carboxylate (Int-96c). MS (EST) [M+H]⁺ m/: 820.

### Step D: 2-(((R)-1-((dimethylamino)methyl)-2,2-difluorocyclopropyl)methoxy)-7-(6-methyl-5-(trifluoromethyl)-1H-indazol-4-yl)-4-((1δ 5R)-1-methyl-3,8-diazabicyclo[3.2.1]octan-3-yl)-7,8-dihydro-5H-pyrano[4,3-d]primidin-5-one (Ex. 96a and Ex. 96b)

A stirred solution of tert-butyl (1S,5R)-3-(2-(((R)-1-((dimethylamino)methyl)-2,2-difluorocyclopropyl)methoxy)-7-(6-methyl-1-(tetrahydro-2H-pyran-2-yl)-5-(trifluoromethyl)-1H-indazol-4-yl)-5-oxo-7,8-dihydro-5H-pyrano[4,3-d]pyrimidin-4-yl)-1-methyl-3,8-diazabicyclo[3.2.1]octane-8-carboxylate (Int-96c) (100 mg, 0.122 mmol) in 4 N HCl in dioxane (1.2 mL) was stirred at 25° C. for 1 h. The mixture was concentrated and purified by reverse phase HPLC (MeCN/water with 0.1% TFA modifier) to give 2-(((R)-1-((dimethylamino)methyl)-2,2-difluorocyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidin-5-one (Ex. 96a, the first eluting isomer) ¹H NMR (400 MHz, MeOD) δ 8.45-8.39 (m, 1H), 7.59 (s, 1H), 6.50 (br d, J=10.6 Hz, 1H), 4.98-4.94 (m, 1H), 4.75 (br d, J=12.5 Hz, 1H), 4.53 (d, J=11.7 Hz, 1H), 4.12 (br s, 1H), 3.78 (br d, J=9.8 Hz, 3H), 3.60 (dd, J=12.9, 18.0 Hz, 1H), 3.44 (d, J=14.1 Hz, 1H), 3.19-3.13 (m, 1H), 3.02 (s, 6H), 2.66 (br d, J=3.5 Hz, 3H), 2.47 (br s, 1H), 2.19 (s, 1H), 2.07-1.94 (m, 2H), 1.93-1.81 (m, 3H), 1.59 (s, 3H). MS (ESI) [M+H]⁺ m/z 636, 2-(((R)-1-((dimethylamino)methyl)-2,2-difluorocyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidin-5-one (Ex. 96b, the second eluting isomer). ¹H NMR (400 MHz, MeOD) δ 8.39 (s, 1H), 7.59 (s, 1H), 6.57 (br d, J=11.0 Hz, 1H), 5.11 (br d, J=15.7 Hz, 1H), 4.73-4.64 (m, 1H), 4.61-4.55 (m, 1H), 4.26 (br s, 1H), 3.81-3.71 (m, 2H), 3.69-3.51 (m, 2H), 3.48-3.38 (m, 2H), 3.18 (br d, J=17.6 Hz, 1H), 3.02 (s, 6H), 2.66 (br d.-=3.1 Hz. 3H), 2.35-2.15 (m, 2H), 1.98 (br s, 2H), 1.85 (br s, 2H), 1.52 (s, 3H₁). MS (ESI) [M+H]1 m/z 636.

### Example 97: 2-((1-(((R)-3-fluoropyrrolidin-1-yl))methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidine (Ex. 97)

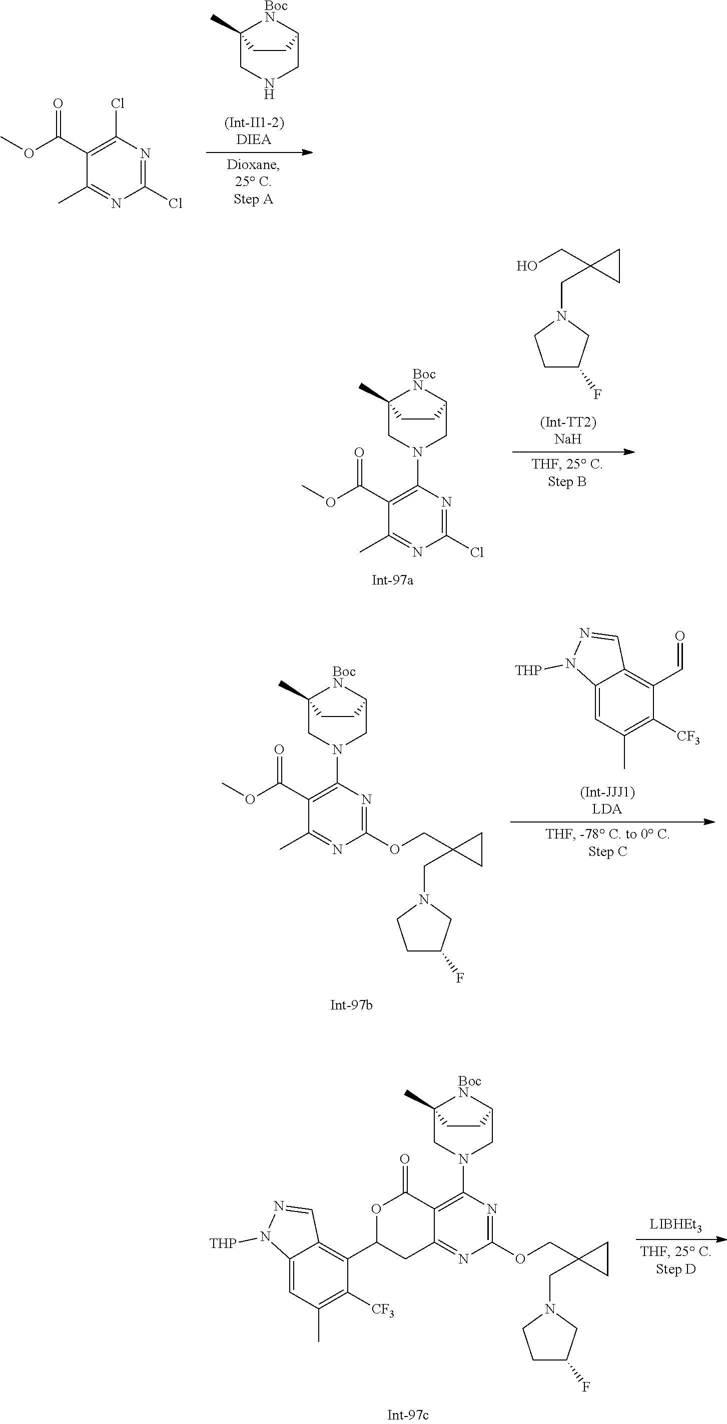

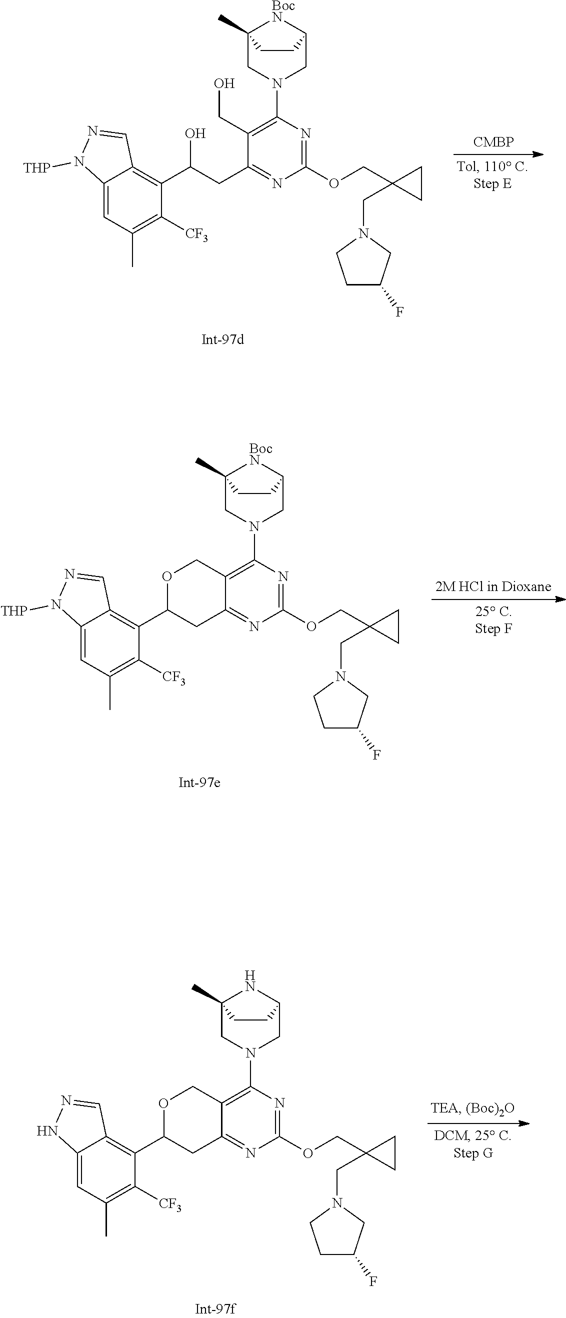

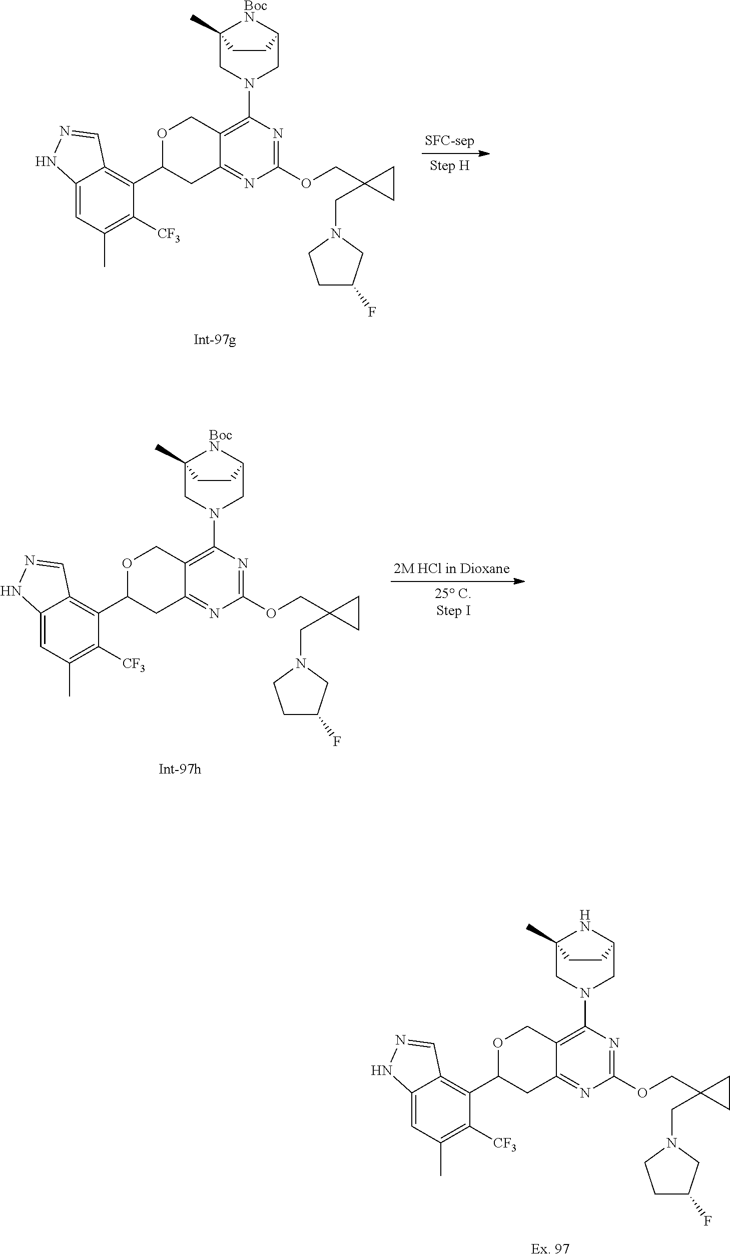 [SMILES: Cc1cc2[nH]ncc2c(C2Cc3nc(OCC4(CN5CC[C@@H](F)C5)CC4)nc(N4C[C@H]5CC[C@@](C)(C4)N5C(=O)OC(C)(C)C)c3CO2)c1C(F)(F)F ; Cc1cc2[nH]ncc2c(C2Cc3nc(OCC4(CN5CC[C@@H](F)C5)CC4)nc(N4C[C@H]5CC[C@@](C)(C4)N5C(=O)OC(C)(C)C)c3CO2)c1C(F)(F)F ; Cc1cc2[nH]ncc2c(C2Cc3nc(OCC4(CN5CC[C@@H](F)C5)CC4)nc(N4C[C@H]5CC[C@@](C)(C4)N5)c3CO2)c1C(F)(F)F]

### Step A: Tert-butyl 01S,5R)-3-(2-chloro-5-(methoxylcarbonyl)-6-methylyprimidin-4:yl)-1-methyl-3,8-diazabicyclo[3.2.1]octane-8-carboxylate (Int-97a)

To a mixture of methyl 2,4-dichloro-6-methylpyrimidine-5-carboxylate (1.11 g, 4.98 mmol) in dioxane (25 mL) were added DIEA (2.17 mL. 12.4 mmol) and tert-butyl (1S,5R)-1-methyl-3,8-diazabicyclo[3.2.1]octane-8-carboxylate (Int-II1-2) (1.24 g, 5.47 mmol). The mixture was stirred at 25° C. for 1 h. The mixture was concentrated and purified by flash silica gel chromatography (Eluent of 20% EtOAc/Pet, ether gradient) to provide tert-butyl (1S,5R)-3-(2-chloro-5-(methoxycarbonyl)-6-methylpyrimidin-4-yl)-1-methyl-3,8-diazabicyclo[3.2.1]octane-8-carboxylate (Int-97a). MS (ESI) [M+H]⁺ m/z 411.

### Step B: Tert-butyl (1S,5R)-3-(2-((1-(((R)-3-fluoropyrrolidin-1-yl)methyl)cyclopropyl)methoxy)-5-(methoxycarbonyl)-6-methylpyrimidin-4-yl)-1-methyl-3,8-diazabicyclo[3.2.1]octane-8-carboxylate (Int-97b)

To a stirred solution of tert-butyl (1S,5R)-3-(2-chloro-5-(methoxycarbonyl)-6-methylpyrimidin-4-yl)-1-methyl-3,8-diazabicyclo[3.2.1]octane-8-carboxylate (Int-97a) (I g, 2 mmol) and (R)-(1-((3-fluoropyrrolidin-1-yl)methyl)cyclopropyl)methanol (Int-TT2) (0.632 g, 3.65 mmol) in THF (20 mL) was added NaH (0.389 g, 9.73 mmol, 60% dispersion in mineral oil) at 0° C. under N₂. After the addition was finished, the reaction was stirred at 25° C. for 2 h. The mixture was concentrated and water (10 mL) was added to the residue and extracted with EtOAc (15 mL×3); the organic layers were washed with saturated aqueous NaCl (20 mL), dried over Na₂SO₄ and concentrated. The residue was purified by flash silica gel chromatography (Eluent of (1-36% Ethyl acetate/Petroleum ether gradient) to provide tert-butyl (1S,5R)-3-(2-((1-(((R)-3-fluoropyrrolidin-1-yl)methyl)cyclopropyl)methoxy)-5-(methoxycarbonyl)-6-methylpyrimidin-4-yl)-1-methyl-3,8-diazabicyclo[3.2.1]octane-8-carboxylate (Int-97b). MS (ESI) [M+H]⁺ m/z 548.

### Step C: Tert-butyl (1S,5R)-3-(2-((1-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-5-oxo-7,8-dihydro-5H-pyrano[4,3-d]pyrimidin-4-yl)-1-methyl-3,8-diazabicyclo[3.2.1]octane-8-carboxylate (Int-97c)

To a mixture of diisopropylamine (0.080 mL, 0.570 mmol) in THF (5 mL) was added nBuLi (2.5 M in THF) (0.228 mL, 0.570 mmol) at −78° C. under N₂. The mixture was stirred at 0° C. for 0.5 h, then the mixture was cooled to −78° C., tert-butyl (1S,5R)-3-(2-((1-(((R)-3-fluoropyrrolidin-1-yl)methyl)cyclopropyl)methoxy)-5-(methoxycarbonyl)-6-methylpyrimidin-4-yl)-1-methyl-3,8-diazabicyclo[3.2.1]octane-8-carboxylate (Int-97b) (260 mg, 0.475 mmol) in THF (1 mL) was added dropwise at −78° C. under N₂. After the mixture was stirred at −78° C. for 0.5 h, 6-methyl-1-(tetrahydro-2H-pyran-2-yl)-5-(trifluoromethyl)-1H-indazole-4-carbaldehyde (Int-JJJ-1) (297 mg, 0.949 mmol) in THF (1 mL) was added at −78° C. The temperature was allowed to warm to 0° C. Then the mixture was stirred at 0° C. for 1 h. The reaction was quenched with sat. NH₄Cl (10 mL). The organic layer was separated and the aqueous layer was re-extracted with EtOAc (10 mL×4). The combined organic layers were washed with brine (30 mL), dried over anhydrous Na₂SO₄, filtered and concentrated. The mixture was concentrated and purified by reverse phase preparative HPLC (MeCN/water with 0.1% ammonium hydroxide modifier) to provide tert-butyl (1S,5R)-3-(2-((l-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-5-oxo-7,8-dihy dro-5H-pyrano[4,3-d]pyrimidin-4-yl)-1-methyl-3,8-diazabicyclo[3.2.1]octane-8-carboxylate (Int-97c). MS (ESI) [M+H]⁺ m/z 828.

### Step D: Tert-butyl (1S,5R)-3-(2-((1-(((R)-3-fluoropyrrolidin-1-yl)methyl)cyclopropyl)methoxy)-6-(2-hydroxy-2-(6-methyl-1-(tetrahydro-2H-pyran-2-yl)-54trifluoromethyl) -1H-indazol-4-ylethyl-5-(hydroxymethyl)pyrimidin-4-yl-1-methyl-3,8-diazabicyclo[3.2.1]octane-8-carboxylate (Int-97d)

To a stirred solution of tert-butyl (1S,5R)-3-(2-((1-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-5-oxo-7,8-dihydro-5H-pyrano[4,3-d]pyrimidin-4-yl)-1-methyl-3,8-diazabicyclo[3.2.1]octane-8-carboxylate (Int-97c) (140 mg, 0.169 mmol) in THF (2 mL) was added lithium triethylborohydride (17.9 mg, 0.169 mmol) at 0° C. under N₂. After the addition was finished, the reaction was stirred at 25° C. for 0.5 h under N₂. Water was added to the mixture (5 mL) and it was extracted with EtOAc (5 mL×3). The organic layers were washed with aqueous saturated NaCl (10 mL), dried over Na₂SO₄ and concentrated. The crude product was purified by prep-TLC using ethyl acetate as eluent to provide tert-butyl (1S,5R)-3-(2-((1-(((R)-3-fluoropyrrolidin-1-yl)methyl)cyclopropyl)methoxy)-6-(2-hydroxy-2-(6-methyl-1-(tetrahydro-2H-pyran-2-yl)-5-(trifluoromethyl)-1H-indazol-4-yl)ethyl)-5-(hydroxymethyl)pyrimidin-4-yl)-1-methyl-3,8-diazabicyclo[3.2.1]octane-8-carboxylate (Int-97d). MS (ESI) [M+H]⁺ m/z 832.

### Step E: Tert-butyl (1S,5R)-3-(2-((I-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-7,8-dihydro-5H-pyrano[4.3-d]pyrimidin-4-yl)-1-methyl-3,8-diazabicyclo[3.2.1]octane-8-carboxyate (Int-97e)

To a stirred solution of tert-butyl (1S,5R)-3-(2-((1-(((R)-3-fluoropyrrolidin-1-yl)methyl)cyclopropyl)methoxy)-6-(2-hydroxy-2-(6-methyl-1-(tetrahydro-2H-pyran-2-yl)-5-(trifluoromethyl)-1H-indazol-4-yl)ethyl)-5-(hydroxymethyl)pyrimidin-4-yl)-1-methyl-3,8-diazabicyclo[3.2.1]octane-8-carboxylate (Int-97d) (170 mg, 0.204 mmol) in toluene (4 mL) was added 2-(tributyl-15-phosphaneyldene)acetonitrile (222 mg, 0.920 mmol) at 25° C. under N₂, and the reaction was stirred at 110° C. for 3 h under N₂. The mixture was concentrated and purified by prep-TLC using (ethyl acetate as eluent) to provide tert-butyl (1S,5R)-3-(2-((1-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97e). MS (ESI) [M+H]⁺ a m/z 814.

### Step F: 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4.3-d]pyrimidine (Int-97f) 970

A solution of tert-butyl (1S,5R)-3-(2-((1-(((R)-3-fluoropyrrolidin-1-yl)methyl)cyclopropyl)methoxy)-7-(6-methyl-1-(tetrahydro-2H-pyran-2-yl)-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97e) (100 mg, 0.123 mmol) in HCl in dioxane (0.4 mL, 4 M) was stirred at 25° C. for 1 h. The mixture was concentrated to provide 2-((I-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidine (Int-97f). MS (ESI) [M+H]⁺ m/z 630.

### Step G: Tert-butyl (1S,5R)-3-(2-((1-(((R)-3-fluoropyrrolidin-1-yl)methyl)cyclopropyl)methoxy)-7-(6-methyl-5-(trifluoromethyl)-1H-indazol-4-yl-7,8-dihydro-5H-pyrano[4.3-d]pyrimidin-4-yl)-1-methyl-3,8-diazabicyclo[3.2.1]octane-8-carboxylate (Int-97g)

To a stirred solution of 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidine (Int-97f) (60 mg, 0.095 mmol) in DCM (0.7 mL) were added Boc₂O (0.033 mL, 0.14 mmol) and TEA (0.020 mL, 0.14 mmol) at 25° C. After the addition was finished, the reaction was stirred at 25° C. for 2 h. The mixture was concentrated and purified by reverse phase using basic modifier to provide tert-butyl (1S,5R)-3-(2-((1-(((R)-3-fluoropyrrolidin-1-yl)methyl)cyclopropyl)methoxy)-7-(6-methyl-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97g). MS (ESI) [M+H]⁺ m/z 730.

### Step H: Tert-butyl (1S,5R)-3-(2-((1-((R)-3-fluoropyrrolidin-1-v)methyl)cyclopropyl)methoxy)-7-(6-methyl-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97h)

The mixture of tert-butyl (1S,5R)-3-(2-((1-(((R)-3-fluoropyrrolidin-1-yl)methyl)cyclopropyl)methoxy)-7-(6-methyl-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97g) (50 mg, 0.069 mmol) was separated by preparative SFC (Column 0; Condition: 0.1% NH₄OH in MeOH) to afford tert-butyl (1 S,5R)-3-(2-((1-(((R)-3-fluoropyrrolidin-1-yl)methyl)cyclopropyl)methoxy)-7-(6-methyl-5-(trifluoromethyl)-1H-indazol-4-yl)-7,8-dihydro-5H-pyrano[4,3-d]pyrimidin-4-yl)- I-methyl-3,8-diazabicyclo[3.2.1]octane-8-carboxylate (Peak 2, the second eluting isomer) (Int-97h). MS (ESI) [M+H]⁺ m/z 730.

### Step 1: 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4.3-d]pyrimidine (Ex. 97)

A stirred solution of tert-butyl (1S,5R)-3-(2-((1-(((R)-3-fluoropyrrolidin-1-yl)methyl)cyclopropyl)methoxy)-7-(6-methyl-5-(trifluoromethyl)-1H-indazol-4-yl)-7,8-dihydro-5H-pyrano[4,3-d]pyrimidin-4-yl)-1-methyl-3,8-diazabicyclo[3.2.1]octane-8-carboxylate (Int-97h) (15 mg, 0.021 mmol) in HCl in dioxane (1 mL, 2 M) was stirred at 25° C. for 1 h. The mixture was cooled, filtered and the solvent was evaporated under reduced pressure to give the crude product. The residue was purified by reverse preparative HPLC (MeCN/water with 0.1% TFA modifier) to provide 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydro-5H-pyrano[4,3-d]pyrimidine (Ex. 97). MS (ESI) [M+H]⁺ m/z 630. ¹H NMR (400 MHz, methanol-d₆) δ 8.36-8.21 (m, 1H), 7.49 (s, 1H), 5.62-5.34 (m, 2H), 5.09 (br d, J=13.9 Hz, 1H), 4.93 (br s, 3H), 4.79-4.54 (m, 1H), 4.50-4.16 (m, 3H), 4.15-4.01 (m, 1H). 3.84 (br d, J=14.7 Hz, 1H), 3.71-3.63 (m, 1H), 3.50-3.44 (m, 1H), 3.41-3.31 (m, 2H), 3.24-3.10 (m, 2H), 2.62 (br d, J=3.4 Hz, 3H), 2.52-1.99 (m, 5H), 1.95-1.75 (m, 1H), 1.54 (s, 3H), 1.37-1.15 (m, 1H), 1.02-0.84 (m, 4H).

### Example 98: 2-((I-(((R)-3-fluoroovrrolidin-1-yl)methyl)cycloprorpyl)methoxy)-7-(6-methyl-5-(trifluoromethyl)-1H-indazol-4-yl)-4-((1 S,5R)-1-methyl-3,8-diazabicyclo[3.2.1]octan-3-yl)-7,8-dihydropyrido[4.3-d]ovrimidin-5(6H)-one (Ex. 98a and Ex. 98b)

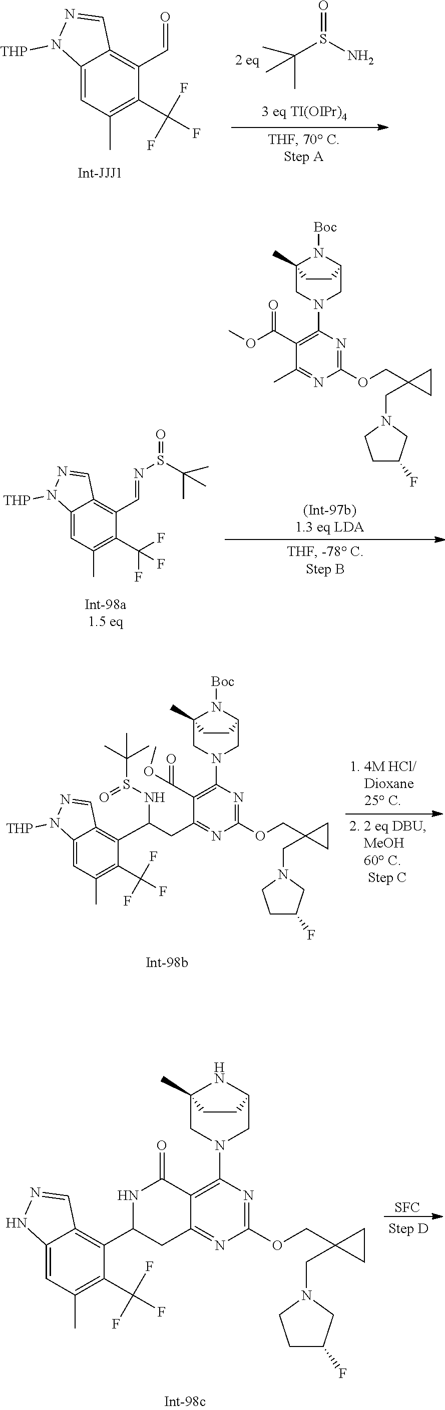

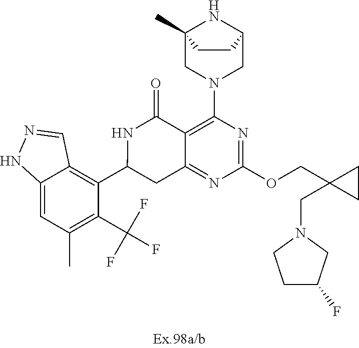 [SMILES: Cc1cc2[nH]ncc2c(C2Cc3nc(OCC4(CN5CC[C@@H](F)C5)CC4)nc(N4C[C@H]5CC[C@@](C)(C4)N5)c3C(=O)N2)c1C(F)(F)F]

### Step A: (E)-2-methyl-N-((6-methyl-4tetrahydro-2H-pyran-2-yl)-5-(trifluoromethyl)-1H-indazol-4-yl)methylene)propane-2-sulfinamide (Int-98a)

A mixture of 6-methyl--(tetrahydro-2H-pyran-2-yl)-5-(trifluoromethyl)-1H-indazole-4-carbaldehyde (Int-JJJ-1) (520 mg, 1.67 mmol), 2-methylpropane-2-sulfinamide (404 mg, 3.33 mmol) and titanium(IV) isopropoxide (1420 mg, 5.00 mmol) in THF (10 mL) was stirred at 70° C. for 12 h. The reaction mixture was concentrated under reduced pressure to give a residue. The residue was purified by flash silica gel chromatography (Eluent of 0-23% EtOAc/Pet, ether gradient) to give (E)-2-methyl-N-((6-methyl-1-(tetrahydro-2H-pyran-2-yl)-5-(trifluoromethyl)-1H-indazol-4-yl)methylene)propane-2-sulfinamide (Int-98a). MS (ESI) [M+H]⁺ m/z 416.

### Step B: Tert-butyl (1S,5R)-3-(6-(2-((tert-butylsulfinyl)amino)-2-(6-methyl-1-(tetrahydro-2H-pyran-2-yl)-5-(trifluoromethyl)-1H-indazol-4-yl)ethyl)-2-((1-(((R)-3-fluoropyrrolidin-1-yl)methyl)cyclopropyl)methoxy)-5-(methoxycarbonyl)pyrimidin-4-yl)-1-methyl-3,8-diazabicyclo[3.2.1]octane-8-carboxylate (Int-98b)

To a solution of tert-butyl (1S,5R)-3-(2-((1-(((R)-3-fluoropyrrolidin-1-yl)methyl)cyclopropyl)methoxy)-5-(methoxycarbonyl)-6-methylpyrimidin-4-yl)-1-methyl-3,8-diazabicyclo[3.2.1]octane-8-carboxylate (Int-97b) (500 mg, 0.913 mmol) in dry THF (10 mL) was added lithium diisopropylamide (1.19 mL, 1.19 mmol, 1 M in THF) at −78° C. under N₂ atmosphere. The mixture was stirred at -78° C. for 20 mins then (E)-2-methyl-N-((6-methyl-1-(tetrahydro-2H-pyran-2-yl)-5-(trifluoromethyl)-1H-indazol-4-yl)methylene)propane-2-sulfinamide (Int-98a) (759 mg, 1.83 mmol) was added. The mixture was stirred at −78° C. for 30 mm. The mixture was quenched with water (5 mL), extracted with DCM (3×5 mL), dried over Na₂SO₄, filtered and the solvent was evaporated under reduced pressure to give the crude product. The residue was purified by reverse phase preparative HPLC (MeCN/water with 0.05% NH₄OH 10 mM NH₄HCO₃) to give tert-butyl (1S,5R)-3-(6-(2-((tert-butylsulfinyl)amino)-2-(6-methyl-1-(tetrahydro-2H-pyran-2-yl)-5-(trifluoromethyl)-1H-indazol-4-yl)ethyl)-2-((1-(((R)-3-fluoropyrrolidin-1-yl)methyl)cyclopropyl)methoxy)-5-(methoxycarbonyl)pyrimidin-4-yl)-1-methyl-3,8-diazabicyclo[3.2.1]octane-8-carboxylate (Int-98b). MS (ESI) [M+H]⁺ m/z 963.

### Step C: 2-((1-(((R)-3-fluoropyrrolidin-1-yl)methyl)cyclopropyl)methoxy)-746-methyl-5-(trifluoromethyl)-1H-indazol-4-yl)-4-((1S,5R)-1-methyl-3,8-diazabicyclo[3.2.1]octan-3-yl)-7,8-dihydropyrido[4,3-d]pyrimidin-5(6H)-one (Int-98c)

A mixture of tert-butyl (1S,5R)-3-(6-(2-((tert-butylsulfinyl)amino)-2-(6-methyl-1-(tetrahydro-2H-pyran-2-yl)-5-(trifluoromethyl)-1H-indazol-4-yl)ethyl)-2-((1-(((R)-3-fluoropyrrolidin-1-yl)methyl)cyclopropyl)methoxy)-5-(methoxycarbonyl)pyrimidin-4-yl)-1-methyl-3,8-diazabicyclo[3.2.]octane-8-carboxylate (Int-98b) (30 mg, 0.031 mmol) and HCl in dioxane (1 mL, 4.00 mmol, 4 M) was stirred at 25° C. for 1 h. The solvent was concentrated, the residue was dissolved in MeOH (1 mL), followed by the addition of DBU (9.39 μl, 0.062 mmol), then the mixture was stirred at 60° C. for 2 h. The mixture was concentrated and purified by reverse preparative HPLC (MeCN/water with 0.1% TFA modifier) to give 2-((1-(((R)-3-fluoropyrrolidin-1-yl)methyl)cyclopropyl)methoxyl-(6-methyl-5-(trifluoromethyl)- H-indazol-4-yl)-4-((1S,5R)-1-methyl-3,8-diazabicyclo[3.2.1]octan-3-yl)-7,8-dihydropyrido[4,3-d]pyrimidin-5(6H)-one (Int-98c). MS (ESI) [M+H]⁺ m/z 643.

### Step D: 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydropyrido[4.3-d]pyrimidin-5(6H)-one (Ex. 98a/b)

2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydropyrido[4,3-d]pyrimidin-5(6H)-one (Int-98c) (15 mg, 0.023 mmol) was separated by SFC (Column A: Condition: 0.1% NH₄OH in EtOH) to give 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydropyrido[4.3-d]pyrimidin-5(6H)-one (the first eluting isomer, Ex. 98a). MS (ESI) [M+H]⁺ m, z 643. ¹H NMR (400 MHz, MeOD) δ 8.34 (s, 1H), 7.54 (s, 1H), 5.69 (dd, J=4.3, 12.9 Hz, 1H), 5.58-5.32 (m, 1H), 4.97 (br d, J=14.5 Hz. 1H), 5.01-4.91 (m, 1H), 4.34 (br s, 2H), 4.20 (br d, J=7.4 Hz, 1H), 4.00 (br s, 1H), 3.76-3.34 (m, 7H), 3.07 (dd, J=4.1, 17.8 Hz. 1H), 2.70-2.58 (m, 3H), 2.38 (br d, J=9.0 Hz, 2H), 2.22-2.07 (m, 1H), 2.06-1.95 (m, 1H), 1.87-1.72 (m, 1H), 1.51 (s, 3H), 1.29 (br s, 2H), 0.92 (br d, J=6.3 Hz, 4H). 2-((1-(((R)-3-fluoropyrrolidin-1-yl)methyl)cyclopropyl)methoxy)-7-(6-methyl-5-(trifluoromethyl)-1H-indazol-4-yl)-4-((1S,5R)-1-methyl-3,8-diazabicyclo[3.2.1]octan-3-yl)-7,8-dihydropyrido[4,3-d]pyrimidin-5(6H)-one (the second eluting isomer, Ex. 98b). MS (ESI) [M+H]⁺ m/z 643. ¹H NMR (400 MHz, MeOD) δ 8.34 (s, 1H). 7.54 (s, 1H). 5.65 (dd, J=4.3, 12.9 Hz, 1H), 5.57-5.30 (m, 1H), 4.41-4.31 (m, 2H), 4.29-3.82 (m, 3H), 3.81-3.64 (m, 2H), 3.61-3.40 (m, 3H), 3.25 (br d, J=13.7 Hz, 2H), 3.13-2.98 (m, 1H), 2.69-2.60 (m, 3H), 2.50 (br s, 3H), 2.21-2.12 (m, 1H), 2.03-1.75 (m, 2H), 1.55 (s, 3H), 1.29 (br s, 2H), 0.98-0.82 (m, 4H).

### Example 99: 5-chloro-4-(8-fluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)pyrido[4,3-d]pyrimidin-7-yl)naphthalen-2-ol (Ex

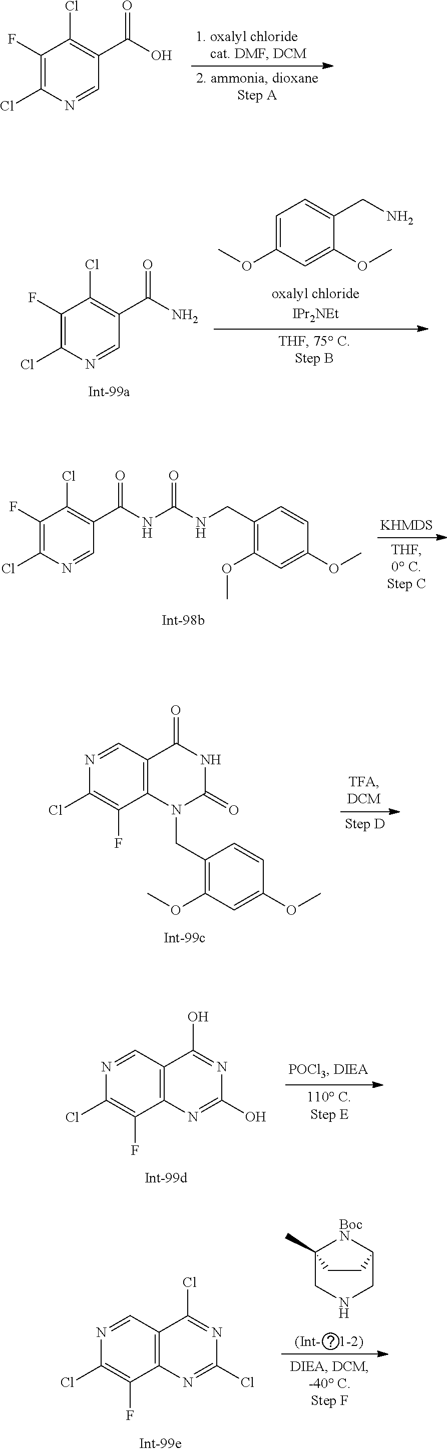

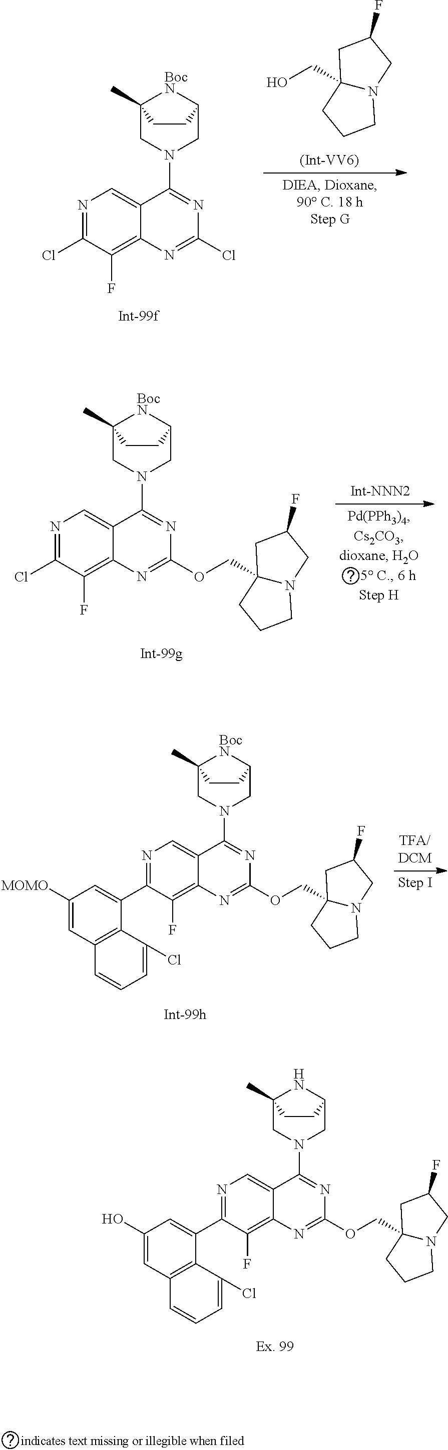

4,6-dichloro-5-fluoronylcodine-3carboxylic acid (2.10 g, 0.01 mol) was dissolved in DCM (20 mL). Oxalyl chloride (1.18 mL, 0.014 mol), followed by 1 drop of DMF was added. The reaction mixture was stirred at room temperature for 4 hours. The mixture was concentrated under reduced pressure. 1.4-dioxane (20 mL) was added and the mixture was cooled to 0° C. 25% Ammonia in dioxane (2.00 mL, 0.016 mol) was added and the reaction was stirred for 10 minutes. The reaction mixture was diluted with ethyl acetate and water. The biphasic mixture was separated, and the organic layer was washed with brine, dried with Na₂SO₄, filtered and concentrated to yield 4,6-dichloro-5-fluoronicotinamide (Int-99a). MS (ESI) [M+H]⁺ m/z 209/211.

### Step B: 4,6-dichloro-N-((2,4-dimethoxybenzyl)carbamoyl)-5-fluoronicotinamide (Int-99b)

4,6-dichloro-5-fluoronicotinamide (Int-99a) (1.98 g, 9.47 mmol) was dissolved in THF (10 mL) and oxalyl chloride (0.96 mL, 11.4 mmol) was added. The reaction mixture was heated to 75° C. for 1 hour. The reaction mixture was cooled and concentrated under reduced pressure. The crude residue was redissolved in THF (10 mL) and cooled to ° C. 2.4-dimethoxyphenyl)methanamine (1.58 mL, 10.7 mmol) and Hunig's base (2 mL) in THF (10 mL) were added and the reaction mixture was stirred for 10 minutes. The reaction was quenched with 10% aqueous H₃PO₄ and diluted with ethyl acetate. The organic layer was washed with saturated aqueous NaHCO₃followed by brine, dried with Na₂SO₄, filtered, and concentrated. The crude solid was taken up in ethyl acetate and diisopropyl ether and the solid was collected by filtration to yield 4,6-dichloro-N-((2,4-dimethoxybenzyl)carbamoyl)-5-fluoronicotinamide (Int-99b). MS (ESI) [M+H] m/z 402.

### Step C: 7-chloro-1-(2,4-dimethoxybenzyl)-8-fluoropyrido[4.3-d]pyrimidine-2.4(kH,3H)-dione (Int-99c)

A suspension of 4,6-dichloro-N-((2,4-dimethoxybenzyl)carbamoyl)-5-fluoronicotinamide (Int-99b) (2.82 g, 7.01 mmol) in THF (20 mL) was cooled to 0° C. in an ice bath. KHMDS (14.7 mL, 14.7 mmol, 1.0 M in THF) was added and the reaction was stirred for one hour at 0° C. The reaction mixture was quenched with 10% aqueous H₃PO₄ and diluted with ethyl acetate. The biphasic mixture was filtered and separated. The organic layer was washed with saturated aqueous NaHCO₃and brine, dried with Na₂SO₄ and filtered through a silica plug, rinsing with ethyl acetate. The solution was concentrated and the crude residue was triturated in diisopropyl ether. The resulting solid was collected by filtration and dried in vacuo at 50° C. to yield 7-chloro-1-(2,4-dimethoxybenzyl)-8-fluoropyrido[4,3-d]pyrimidine-2,4(1H,3H)-dione (Int-99c). MS (ESI) [M+H]⁺ m/z 366.

### Step D: 7-chloro-8-fluoropyrido[4,3-d]pyrimidine-2,4(1H,3H)-dione (Int-99d)

To a mixture of TFA (0.6 mL, 7.79 mmol) in DCM (2.4 mL) was added 7-chloro-1-(2,4-dimethoxybenzyl)-8-fluoropyrido[4,3-d]pyrimidine-2,4(/H,31)-dione (Int-99c) (300 mg, 0.820 mmol) at 25° C. The reaction was stirred at 25° C. for 1 h. The solvent was evaporated under reduced pressure to give a solid which was washed with H₂O (15 mL) and (EtOAc/Pet, ether=3:1), and the solid was collected and dried in vacuo to give 7-chloro-8-fluoropyrido[4,3-d]pyrimidine-2,4(1H,3H)-dione (Int-99d). MS (ESI) [M+H]⁺ m/z 216.

### Step E: 2,4,7-trichloro-8-fluoropyrido[4.3-d]pyrimidine (Int-99e)

To a mixture of POCl₃ (3.00 mL, 32.2 mmol) and DIEA (0.718 mL, 4.11 mmol) at 0° C. was added 7-chloro-8-fluoropyrido[4,3-d]pyrimidine-2,4(/H,3H)-dione (Int-99d) (286 mg, 0.823 mmol). The reaction was stirred at 110° C. for 2 h. The solvent was evaporated under reduced pressure to give 2,4,7-trichloro-8-fluoropyrido[4,3-d]pyrimidine (Int-99e) which was used without further purification.

### Step F: Tert-butyl (1S,5R)-3-(2,7-dichloro-8-fluoropyrido[4,3-d]pyrimidin-4-Y1)-1-methyl-3,8-diazabicyclo[3.2.1]octane-8-carboxylate (Int-99f)

To a solution of 2,4,7-trichloro-8-fluoropyrido[4,3-d]pyrimidine (Int-99e) (208 mg, 0.82 mmol) in DCM (2 mL) was added DIEA (0.43 mL, 2.47 mmol) and tert-butyl (1S,5R)-1-methyl-3,8-diazabicyclo[3.2.1]octane-8-carboxylate (Int-II1-2) (242 mg, 1.07 mmol) at −40° C. The reaction was stirred at −40° C. for 20 min. The reaction mixture was purified directly by preparative Prep-TLC (Pet, ether/EtOAc=3:1) to give tert-butyl (1S,5R)-3-(2,7-dichloro-8-fluoropyrido[4,3-d]pyrimidin-4-yl)-1-methyl-3,8-diazabicyclo[3.2.1]octane-8-carboxylate (Int-99f). MS (ESI) [M+H]⁺ m/z 442.

### Step G: Tert-butyl (1S,5R)-3-(7-chloro-8-fluoro-2-(((2S,7aR)-2-fluorotetrahydro-1H-pyrrolizin-7a(5H)-yl)methoxy)pyrido[4,3-d]pyrimidin-4-yl)-1-methyl-3,8-diazabicyclo[3.2.1]octane-8-carboxylate (Int-99)

To a mixture of ((2S,7aR)-2-fluorotetrahydro-1H-pyrrolizin-7a(5H)-yl)methanol (Int-A18) (76 mg, 0.475 mmol) in dioxane (1 mL) were added N-ethyl-N-isopropylpropan-2-amine (0.083 mL, 0.475 mmol) and tert-butyl (I S,5R)-3-(2,7-dichloro-8-fluoropyrido[4,3-d]pyrimidin-4-yl)-1-methyl-3,8-diazabicyclo[3.2.1]octane-8-carboxylate (Int-99f) (70 mg, 0.158 mmol) at 25 C. The reaction was stirred at 90° C. for 16 h. The reaction mixture was purified directly by preparative Prep-TLC (Pet, ether/EtOAc=1:1) to give tert-butyl (1S,5R)-3-(7-chloro-8-fluoro-2-(((2S,7aR)-2-fluorotetrahydro-1H-pyrrolizin-7a(5H)-yl)methoxy)pyrido[4,3-d]pyrimidin-4-yl)-1-methyl-3,8-diazabicyclo[3.2.1]octane-8-carboxylate (Int-99g). MS (ESI) [M+H]⁺ m, z 565.

### Step H: Tert-butyl (1S,5R)-3-(7-(8-chloro-3-(methoxymethoxy)naphthalen-1-yl-8-fluoro-2-(((2S,7aR)-2-fluorotetrahydro-1H-pyrrolizin-7a(5H)-yl)methoxy)pyrido[4.3-d]pyrimidin-4-yl)-1-methyl-3,8-diazabicyclo[3.2.1]octane-8-carboxylate (Int-99h)

To a solution of tert-butyl (1S,5R)-3-(7-chloro-8-fluoro-2-(((2S,7aR)-2-fluorotetrahydro-1H-pyrrolizin-7a(5H)-yl)methoxy)pyrido[4,3-d]pyrimidin-4-yl)-1-methyl-3,8-diazabicyclo[3.2.1]octane-8-carboxylate (Int-99g) (25 mg, 0.044 mmol) in 1.4-dioxane (0.5 mL) and water (0.10 mL) were added potassium (8-chloro-3-(methoxymethoxy)naphthalen-1-yl)trifluoroborate (Int-N2) (97 mg, 0.088 mmol), Cs₂CO₃ (43.2 mg, 0.133 mmol) and Pd(Ph₃P)₄ (5.11 mg, 4.42 μmol) at 25° C. under N₂ atmosphere. The reaction was stirred at 100° C. for 3 h. The solvent was evaporated under reduced pressure to give the crude which was purified by reverse phase preparative HPLC (MeCN/water with 0.1% TFA modifier) to give tert-butyl (1S,5R)-3-(7-(8-chloro-3-(methoxymethoxy)naphthalen-1-yl)-8-fluoro-2-(((2S,7aR)-2-fluorotetrahydro-1H-pyrrolizin-7a(5H)-yl)methoxy)pyrido[4,3-d]pyrimidin-4-yl)-1-methyl-3,8-diazabicyclo[3.2.1]octane-8-carboxylate (Int-99h). MS (ESI) [M+H]⁺m/z, 751.

### Step 1: 5-chloro-4-(8-fluoro-2-(((2S,7aR)-2-fluorotetrahydro-1H-pyrrolizin-7a(5H)-yl)methoxy)-4-((1S,5R)-1-methyl-3,8-diazabicyclo[3.2.1]octan-3-yl)pyrido[4,3-d]pyrimidin-7-yl)naphthalen-2-ol (Ex. 99)

To a solution of TFA (0.1 mL, 1.30 mmol) in DCM (0.3 mL) was added tert-butyl (1S,5R)-3-(7-(8-chloro-3-(methylmethoxy)naphthalen-1-yl)-8-fluoro-2-(((2S,7aR)-2-fluorotetrahydro-1H-pyrrolizin-7a(5H)-yl)methoxy)pyrido[4,3-d]pyrimidin-4-yl)-1-methyl-3,8-diazabicyclo[3.2.1]octane-8-carboxylate (Int-99h) (15 mg, 0.020 mmol) at 25° C. The reaction was stirred at 25° C. for 0.5 h. The solvent was evaporated under reduced pressure to give the crude product which was purified by reverse phase preparative HPLC (MeCN/water with 0.1% TFA modifier) to give 5-chloro-4-(8-fluoro-2-(((2S,7aR)-2-fluorotetrahydro-1H-pyrrolizin-7a(5H)-yl)methoxy)-4-((1S,5R)-1-methyl-3,8-diazabicyclo[3.2.1]octan-3-yl)pyrido[4,3-d]pyrimidin-7-yl)naphthalen-2-ol (Ex. 99). MS (ESI) [M+H]⁺ nm/z 607. ¹H NMR (400 MHz, CD₃OD) δ 9.14 (d, J=2.7 Hz, 1H), 7.77 (dd, J=1.4, 8.0 Hz, 1H), 7.46-7.28 (m, 3H), 7.16 (d, J=1.6 Hz, 1H), 5.69-5.46 (m, 1H), 4.94 (br d, J=14.5 Hz, 1H), 4.84-4.72 (m, 2H), 4.29 (br t, J=6.8 Hz, 1H), 4.10-3.80 (m, 5H). 3.46 (dt, J=5.9, 10.6 Hz. 1H), 2.83-2.00 (m, 10H), 1.97-1.82 (m, 1H), 1.62 (d, J=8.2 Hz, 3H).

### Example 100: 5-chloro-4-(2-(((R)-1-((dimethylamino)methyl)-2,2-difluorocyclopropyl)methoxyl)-8-fluoro-4-((1S,5R)-1-methyl-3,8-diazabicyclo[3.2.1]octan-3-yl)pyrido[4,3-d]pyrimidin-7-yl)naphthalen-2-ol (Ex. 100)

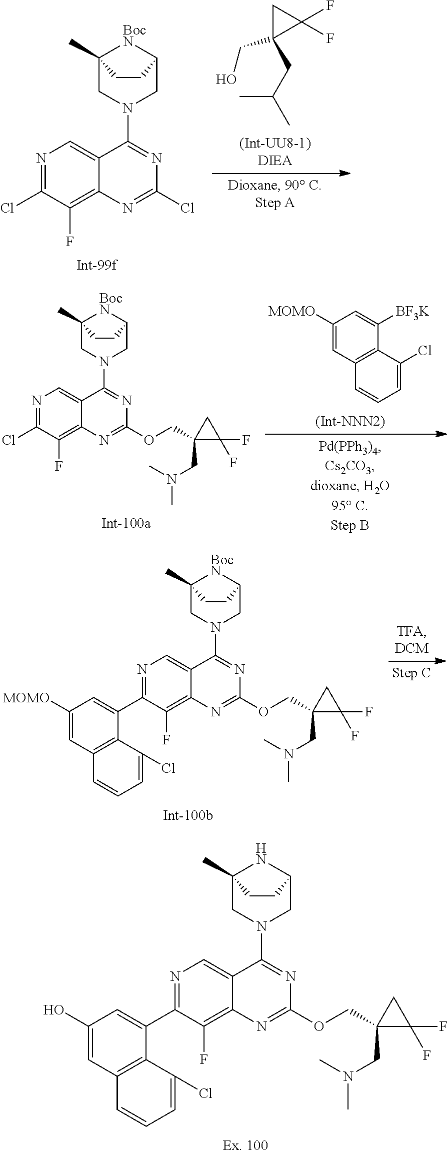

### Step A: Tert-butyl (1S,5R)-3-(7-chloro-2-(((R)-1-(dimethylamino)methyl)-2,2-difluorocyclopropyl)methoxy)-8-fluoropyrido[4,3-d]pyrimidin-4-yl)-1-methyl-3,8-diazabicyclo[3.2.1]octane-8-carboxylate (Int-100a)

To a mixture of (R)-(1-((dimethylamino)methyl)-2,2-difluorocyclopropyl)methanol (Int-UU8-1) (54.7 mg. 0.271 mmol) in 1.4-dioxane (0.5 mL) were added DIEA (0.063 mL, 0.362 mmol) and tert-butyl (1S,5R)-3-(2,7-dichloro-8-fluoropyrido[4,3-d]pyrimidin-4-yl)-1-methyl-3,8-diazabicyclo[3.2.1]octane-8-carboxylate (Int-99f) (40 mg, 0.09 mmol) at 25° C. Then the reaction was stirred at 90° C. for 16 h. The reaction mixture was purified directly by Prep-TLC (Pet, ether/EtOAc=1:1) to give tert-butyl (1S,5R)-3-(7-chloro-2-(((R)-1-((dimethylamino)methyl)-2,2-difluorocyclopropyl)methoxy)-8-fluoropyrido[4,3-d]pyrimidin-4-yl)-1-methyl-3,8-diazabicyclo[3.2.1]octane-8-carboxylate (Int-100a). MS (ESI) [M+H]⁺ m/z 571.

### Step B: Tert-butyl (1S,5R)-347-(8-chloro-3-(methoxymethoxy)naphthalen-1-yl)-2-(((R)-1-(dimethylamino)methyl)-2,2-difluorocyclopropyl)methoxy)-8-fluoropyrido[4,3-d₁pyrimidin-4-v])-1-methyl-3,8-diazabicyclo[3.2.1]octane-8-carboxylate (Int-100b)

To a solution of tert-butyl (1S,5R)-3-(7-chloro-2-(((R)-1-((dimethylamino)methyl)-2,2-difluorocyclopropyl)methoxy)-8-fluoropyrido[4,3-d]pyrimidin-4-yl)-1-methyl-3,8-diazabicyclo[3.2.1]octane-8-carboxylate (Int-100a) (30 mg, 0.053 mmol) in 1.4-dioxane (0.5 mL) and water (0.10 mL) were added potassium (8-chloro-3-(methoxymethoxy)naphthalen-1-yl)trifluoroborate (Int-NNN2) (115 mg, 0.105 mmol), Cs₂CO₃ (51.4 mg, 0.158 mmol) and Pd(Ph₃P)₄ (6.07 mg, 5.25 μmol) at 25° C. under N₂ atmosphere. The reaction was stirred at 100° C. for 3 h. The solvent was evaporated under reduced pressure to give the crude product that was purified by reverse phase preparative HPLC (MeCN/water with 0.1% TFA modifier) to give tert-butyl (1S,5R)-3-(7-(8-chloro-3-(methoxymethoxy)naphthalen-1-yl)-2-(((R)-1-((dimethylamino)methyl)-2,2-difluorocyclopropyl)methoxy)-8-fluoropyrido[4,3-d]pyrimidin-4-yl)-1-methyl-3,8-diazabicyclo[3.2.1]octane-8-carboxylate (Int-100b). MS (ESI) [M+H]⁺ m/z 757.

### Step C: 5-chloro-4-(2-(((R)-1-((dimethylamino)methyl)-2,2-difluorocyclopropyl)methoxy)-8-fluoro-4-((1S,5R)-1-methyl-3,8-diazabicyclo[3.2.1]octan-3-yl)pyrido[4.3-d]pyrimidin-7-yl)naphthalen-2-ol (Ex

To a solution of TFA (0.1 mL, 1.298 mmol) in DCM (0.3 mL) was added tert-butyl (1S,5R)-3-(7-(8-chloro-3-(methoxymethoxy)naphthalen-1-yl)-2-(((R)-1-((dimethylamino)methyl)-2,2-difluorocyclopropyl)methoxy)-8-fluoropyrido[4,3-d]pyrimidin-4-yl)-1-methyl-3,8-diazabicyclo[3.2.1]octane-8-carboxylate (Int-100b) (16 mg, 0.021 mmol) at 25° C. The reaction was stirred at 25° C. for 0.5 h. The solvent was evaporated under reduced pressure to give the crude product which was purified by reverse phase preparative HPLC (MeCN/water with 0.1% TFA modifier) to give 5-chloro-4-(2-(((R)-1-((dimethylamino)methyl)-2,2-difluorocyclopropyl)methoxy)-8-fluoro-4-((1S,5R)-1-methyl-3,8-diazabicyclo[3.2.1]octan-3-yl)pyrido[4,3-d]pyrimidin-7-yl)naphthalen-2-ol (Ex. 100). MS (ESI) [M+H]⁺ m/z 613. ¹H NMR (400 MHz, MeOD) δ 9.13 (d, J=2.0 Hz, 1H), 7.77 (dd, J=1.2, 7.8 Hz, 1H), 7.45-7.29 (m, 3H), 7.16 (t, J=2.0 Hz, 1H), 4.91 (br s, 1H), 4.83-4.74 (m, 2H), 4.70-4.57 (m, 1H), 4.28 (br s, 1H), 4.00-3.67 (m, 3H). 3.47 (dd, J=7.6, 14.3 Hz, 1H), 3.02 (s, 6H), 2.34-1.77 (m, 6H), 1.62 (d, J=6.3 Hz, 3H).

### Ex 224: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronauhthalen-2-ol

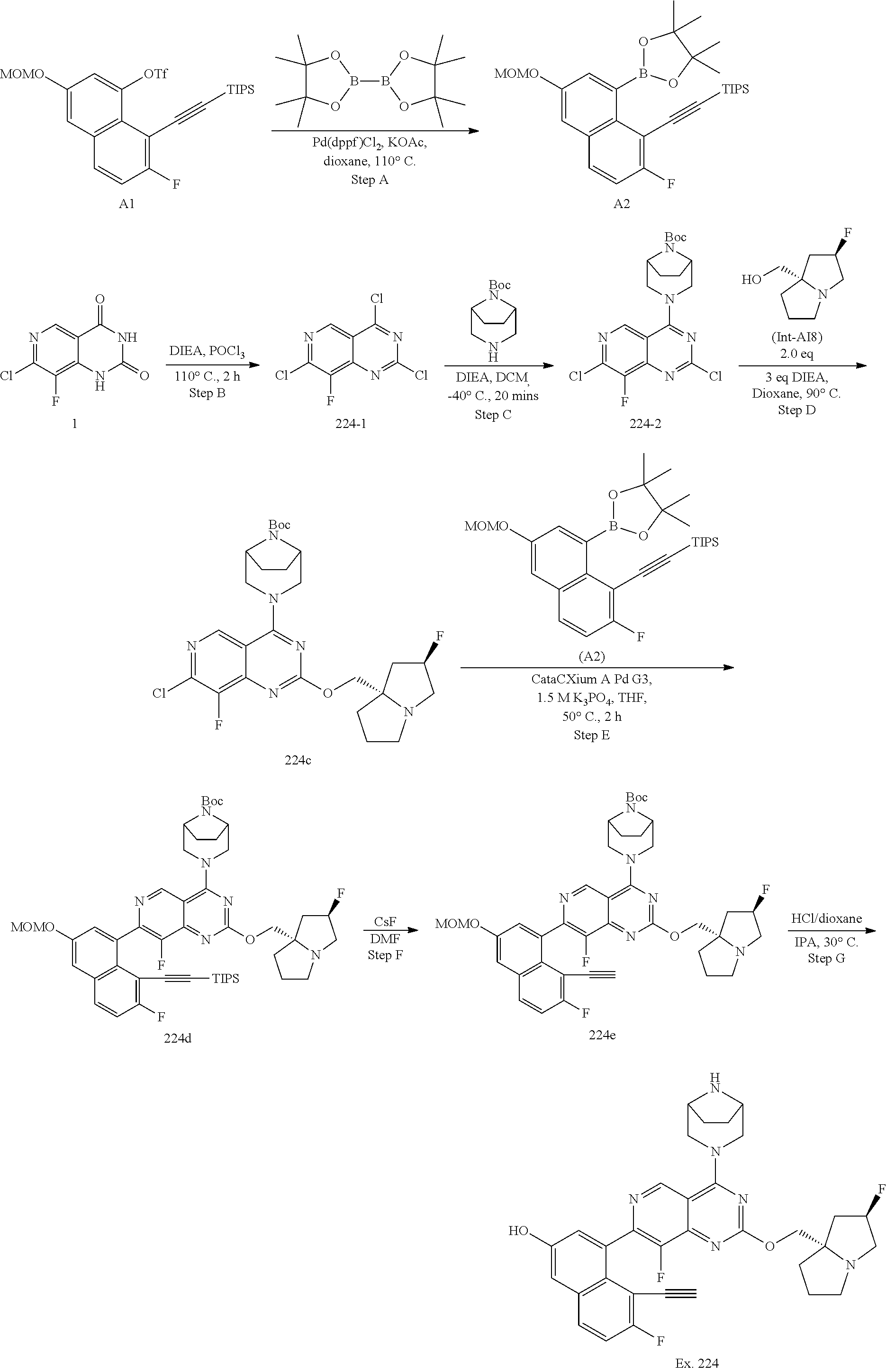

### Step A: ((2-fluoro-6-(methoxlymethoxy)-8-(4,4,5,5-tetramethyl-1,3,2-dioxaborolan-2-yl)naphthalen-1-yl)ethynyl)triisopropylsilane (A2)

To a solution of 7-fluoro-3-(methoxymethoxy)-8-((triisopropylsilyl)ethynyl)naphthalen-1-yl trifluoromethanesulfonate (15 g, 28.1 mmol) (A1), 4,4,4′4′,5,5,5′,5′-octamethyl-2,2′-bi(1,3,2-dioxaborolane) (13.32 g, 52.5 mmol), [1.1′-bis(diphenylphosphino)ferrocene] dichloropalladium(II) (2.053 g, 2.81 mmol) and potassium acetate (6.88 g, 70.1 mmol) in dioxane (150 mL) was stirred at 110° C. under N₂ for 15 h. The reaction was monitored by TLC (Pet, ether/EtOAc=20:1). The mixture was concentrated, the residue was purified by flash silica gel chromatography 330 g column, Eluent of Pet, ether gradient, to give ((2-fluoro-6-(methoxymethoxy)-8-(4,4,5,5-tetramethyl-1,3,2-dioxaborolan-2-yl)naphthalen-1-yl)ethynyl)triisopropylsilane. ¹H NMR (400 MHz, CDCl₃) 5 7.69-7.64 (m, 1H), 7.51 (d, J=2.5 Hz, 1H), 7.38 (d, J=2.6 Hz, 1H), 7.23 (t, J=8.8 Hz, 1H), 5.29-5.26 (m, 2H), 3.51 (s, 3H), 1.44 (s, 12H), 1.18-1.14 (m, 21H).

### Step B: 2,4,7-trichloro-8-fluoropyrido[4.3-d]pyrimidine (Int-224-1)

To a mixture of 7-chloro-8-fluoropyrido[4,3-d]pyrimidine-2,4(1H,3H)-dione (7.5 g, 34.8 mmol) in toluene (8 mL) was added DIEA (18.23 mL, 104 mmol) and POCl₃ (9.73 mL, 104 mmol) at 0° C. The reaction was stirred at 110° C. for 2 h and monitored by LCMS, The solvent was evaporated under reduced pressure, and the residue was azeotroped with CHCl₃ to give 2,4,7-trichloro-8-fluoropyrido[4,3-d]pyrimidine (8.78 g, 34.8 mmol, 100% yield) as a black oil, which was used to the next step directly.

### Step C: tert-butyl (1R,5S)-3-(2,7-dichloro-8-fluoropyrido[4,3-d]pyrimidin-4-yl)-3,8-diazabicyclo[3.2.1]octane-8-carboxylate (Int-224-2)

To a mixture of 2,4,7-trichloro-8-fluoropyrido[4,3-d]pyrimidine (Int-224-1) (8.78 g, 34.8 mmol) and DIEA (60.7 mL, 348 mmol) in DCM (80 mL) at -40° C. was added tert-butyl (1R,5S)-3,8-diazabicyclo[3.2.1]octane-8-carboxylate (7.75 g, 36.5 mmol), and the reaction was stirred at −40° C. for 20 min. The reaction was monitored by TLC and LCMS showed the starting material was consumed and a new spot was formed. The reaction mixture was quenched by H₂O (40 mL), extracted with DCM (3×40 mL). The combined organic layer was washed with brine (2×30 mL), dried over Na₂SO₄, filtered and the solvent was evaporated under reduced pressure. The crude was purified by flash silica gel chromatography 220 g column. Eluent of 0-35% EtOAc/Pet, ether gradient, to give tert-butyl (1R,5S)-3-(2,7-dichloro-8-fluoropyrido[4,3-d]pyrimidin-4-yl)-3,8-diazabicyclo[3.2.1]octane-8-carboxylate. MS (ESI): m/z (M+H)⁺ 428.

### Step D: Tert-butyl (1R,5S)-3-(7-chloro-8-fluoro-2-(((2R,7aS)-2-fluorotetrahydro-1H-pyrrolizin-7a(5H)-yl)methoxy)pyrido[4.3-d]pyrimidin-4-yl)-3,8-diazabicyclo[3.2.1]octane-8-carboxylate (Int-224c)

To a solution of tert-butyl (1R,5S)-3-(2,7-dichloro-8-fluoropyrido[4,3-d]pyrimidin-4-yl)-3,8-diazabicyclo[3.2.1]octane-8-carboxylate (Int-224-2) (7.8 g, 18.21 mmol) and DIEA (9.54 mL, 54.6 mmol) in dioxane (60 mL) was added ((2R,7aS)-2-fluorotetrahydro-1H-pyrrolizin-7a(5H)-yl)methanol (Int-A18) (5.80 g, 36.4 mmol) at 25° C., and the reaction was stirred at 90° C. for 12 h. The reaction was monitored by LCMS, The reaction mixture was quenched with water (50 mL), and the resulting mixture was extracted with EtOAc (60 mL×3). The combined organic phase was dried over anhydrous Na₂SO₄, filtered and concentrated in vacuo to give the crude which was purified by flash silica gel chromatography 80 g column, Pet, ether/EtOAc=13/1, 30 mm, dry loaded, to give tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate. MS (ESI): m/z (M+H)⁺ 551.

### Step E: Tert-butyl (1R,5S)-3-(8-fluoro-7-(7-fluoro-3-(methoxymethoxy)-8-((triisopropylsilyl)ethyl)naphthalen-1-yl)-2-(((2R,7aS)-2-fluorotetrahydro-1H-pyrrolizin-7a(5H)-yl)methoxy)pyrido[4.3-d]pyrimidin-4-yl)-3,8-diazabicyclo[3.2.1]octane-8-carboxylate (Int-224d)

To a solution of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (Int-224-c) (3.3 g, 5.99 mmol) in THF (30 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (A2) (4.14 g, 8.08 mmol), potassium phosphate tribasic (17.97 mL, 17.97 mmol) (1 M in H₂O) and methanesulfonato[(di(I-adamantyl)-n-butylphosphine)-2-(2′-amino-1,1′-biphenyl)]palladium(II) (0.872 g, 1.198 mmol) at 25° C. The reaction was stirred at 50° C. for 2 h under N₂ and monitored by LCMS, The reaction mixture was quenched with water (30 mL) and extracted with EtOAc (30 mL×3). The combined organic phase was dried over anhydrous Na₂SO₄, filtered and concentrated in vacuo to give the crude which was purified by flash silica gel chromatography (ISCO®; 40 g column, Eluent of 0-78% EtOAc/Pet, ether gradient, dry loaded, to give tert-butyl (1R,5S)-3-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate. MS (EST): m/z (M+H)⁺ 901.

### Step F: Tert-butyl (1R,5S)-3-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4.3-d]pyrimidin-4-yl)-3,8-diazabicyclo[3.2.1]octane-8-carboxylate (Int-224e)

To a solution of tert-butyl (1R,5S)-3-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (Int-224d) (3.5 g, 3.88 mmol) in DMF (20 mL) was added CsF (2.95 g, 19.42 mmol) at 25° C. The reaction was stirred at 25° C. for 2 h under N₂ and was monitored by LCMS, The reaction mixture was quenched with H₂O (30 mL) and extracted with EtOAc (20 mL×3). The combined organic phase was dried over anhydrous Na₂SO₄, filtered and concentrated in vacuo to give the crude which was purified by flash silica gel chromatography 40 g column, Eluent of 0-5% MeOH/DCM gradient, dry loaded, to give tert-butyl (1R,5S)-3-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate. MS (ESI): m/z (M+H)⁺ 745.

### Step G: 44-(4(R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (Ex.224)

To a solution of HCl/dioxane (50 mL, 609 mmol) (4 M in dioxane) in IPA (50 mL) was added tert-butyl (1R,5S)-3-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl))methoxy)pyrido[4,3-d]pyrimidin-4-yl)-3,8-diazabicyclo[3.2.1]octane-8-carboxylate (Int-224e) (6 g, 8.06 mmol) at 25° C. The reaction was stirred at 30° C. for 5 h under N₂ and monitored by LCMS, Then the solvent was evaporated under reduced pressure at 30° C. to give the crude product which was purified by preparative HPLC (Column: phenomenex Gemini-NX C18 150*40 mm*5 um Condition water (10 mM-NH₄HCO3)-ACN Begin B 30 End B 60 Gradient Time (min) 10 100% B Hold Time 2 Flow Rate (mL/min) 60) to give 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol. MS (EST): m,: (M+H)⁺ 601. Ex. 224: ¹H NMR (400 MHz, MeOD) δ 9.01 (s, 1H), 7.86 (dd, J=5.7, 9.1 Hz, 1H), 7.38-7.28 (m, 2H), 7.21 (d, J=2.5 Hz, 1H), 5.40-5.22 (m, 1H), 4.61 (q, J=11.1 Hz, 2H), 4.33-4.19 (m, 2H), 3.77-3.62 (m, 4H), 3.36 (d, J=7.2 Hz, 1H), 3.29-3.17 (m, 3H), 3.01 (dt, J=5.7, 9.4 Hz, 1H), 2.40-2.09 (m, 3H), 2.05-1.94 (m, 2H), 1.94-1.75 (m, 5H).

### Example 101: 4-(6-((1-((dimethylamino)methyl)cycloropyl)methoxy)-8-((1S,5R)-1-methyl-3,8-diazabicyclo[3.2.1]octan-3-yl)pyrimido[5,4-c]pyridazin-3-yl)naphthalen-2-ol (Ex. 101)

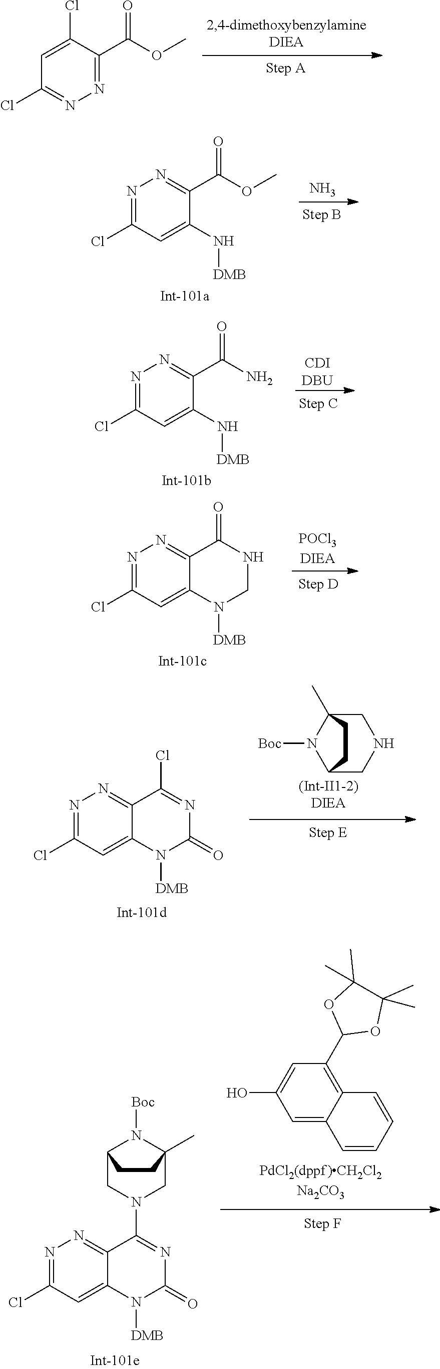

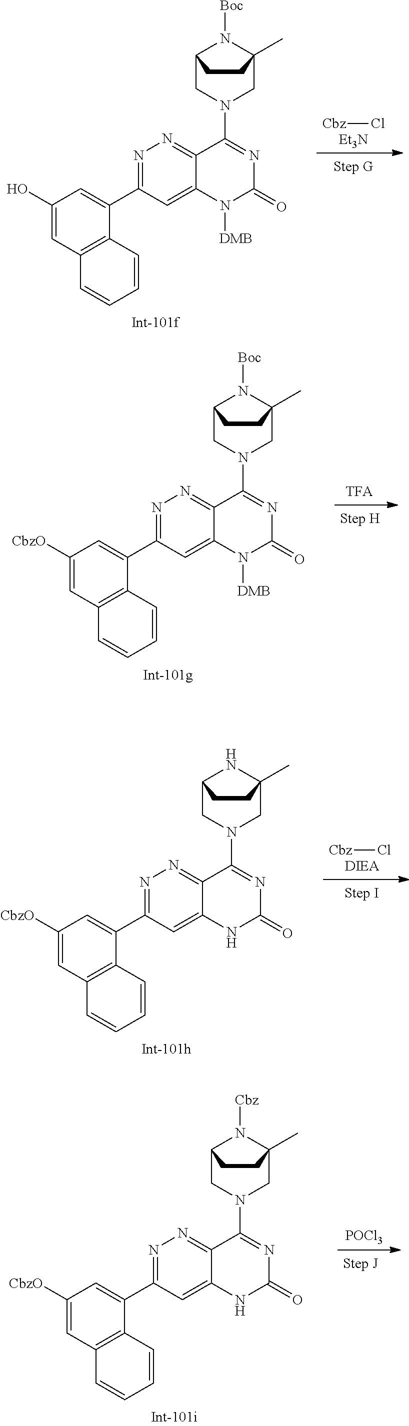

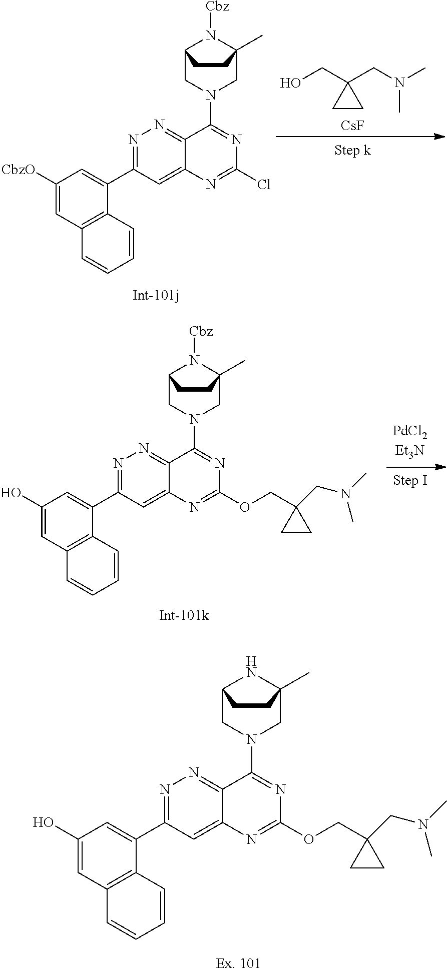

### Step A: Methyl 6-chloro-4-((2,4-dimethoxybenzyl)amino)p3ridazine-3-carboxylate (Int-101a)

Methyl-4,6-dichloropyridazine-3-carboxylate (12.5 g, 60.4 mmol) in acetonitrile (75 mL) was treated with N,N-diisopropylethylamine (21.1 mL, 121 mmol) and 2,4-dimethoxybenzylamine (10.0 mL, 66.4 mmol) at ambient temperature. The mixture was stirred at ambient temperature for 24 h resulting in a thick slurry. The solid was collected by vacuum filtration and washed with fresh acetonitrile (3×10 mL) and then dried under vacuum to afford the intended product. Additional crops were obtained by concentrating the combined filtrate and washings to afford a thick amber-colored syrup that was purified by column chromatography on silica gel (0 to 100% EtOAc/hexanes) to afford methyl 6-chloro-4-((2,4-dimethoxybenzyl)amino)pyridazine-3-carboxylate (Int-101a). The solid was dissolved in hot acetonitrile and then aged at −20° C. for 24 h. The solid was collected by vacuum filtration and washed with acetonitrile to afford additional crops of Int-101a. MS (EST) [M+H]⁺ m/z 338/340.

### Step B: 6-chloro-4-((2,4-dimethoxybenzyl)amino)pyridazine-3-carboxamide (Int-101b)

A slurry of methyl 6-chloro-4-((2,4-dimethoxybenzyl)amino)pyridazine-3-carboxylate (Int-101a) (5.00 g, 14.8 mmol) in a solution of ammonia in methanol (7M, 55 mL, 385 mmol) in a sealed flask was heated to 50° C. for 20 h. The slurry was then concentrated and dried under vacuum to afford 6-chloro-4-((2,4-dimethoxylbenzyl)amino)pyridazine-3-carboxamide (Int-101b). MS (ESI) [M+H]⁺ m/z 323/325.

### Step C: 3-chloro-5-(2,4-dimethoxybenzyl)pyrimido[5,4-c]pyridazine-6.8(5H,7H)-dione (Int-101c)

A suspension of 6-chloro-4-((2,4-dimethoxybenzyl)amino)pyridazine-3-carboxamide (Int-101b) (4.80 g, 14.9 mmol) and CDI (3.62 g, 22.3 mmol) in DCM (35.0 mL) was treated at room temperature with 1.8-diazabicyclo(5.4.0)undec-7-ene (3.36 mL, 22.3 mmol). The mixture was heated at 35° C. for 2 h and then cooled to ambient temperature. The crude mixture was directly loaded onto a silica gel column. Purification by column chromatography on silica gel (0 to 10% methanol/DCM) afforded a solid. The solid was dissolved in boiling acetonitrile (125 mL). The solution was allowed to cool to rt and placed in a −20° C. freezer for 30 minutes. The solids were collected by vacuum filtration, washed with acetonitrile and dried under vacuum to afford 3-chloro-5-(2,4-dimethoxybenzyl)pyrimido[5,4-c]pyridazine-6.8(5H,7H)-dione (Int-101c). Additional crops were obtained by concentrating the combined liquor and washings and then dissolving the residue in hot acetonitrile. The solution was allowed to cool to ambient temperature and placed in a −20° C. freezer for 1 8h. Seed from the first crop was added after 30 minutes. The solid was collected by vacuum filtration and washed with acetonitrile to afford additional Int-101c. MS (ESI) [M+H]⁺ m/z 349/350.

### Step D: 3.8-dichloro-5-(2,4-dimethoxybenzyl)pyrimido[5,4-c]pyridazin-6(5H)-one (Int-101d)

3-chloro-5-(2,4-dimethoxybenzyl)pyrimido[5,4-c]pyridazine-6.8(5H,7H)-dione (Int-101c) (1000 mg, 2.87 mmol) in acetonitrile (10.0 mL) was treated at ambient temperature with N,N-diisopropylethylamine (2.50 mL, 14.34 mmol) and phosphorous oxychloride (0.54 mL, 5.73 mmol) and heated at 65° C. for 1 hour, cooled to ambient temperature and concentrated. The residue was treated with DCM (20 mL) and concentrated to afford 3.8-dichloro-5-(2,4-dimethoxybenzyl)pyrimido[5,4-c]pyridazin-6(5H)-one (Int-101d) which was used without further purification. MS (EST) [M+H]⁺ m/z 367/369.

### Step E: Tert-butyl (1S,5R)-3-(3-chloro-5-(2,4-dimethoxybenzyl)-6-oxo-5,6-dihydropyrimido[5,4-c]pyridazin-8-yl)-1-methyl-3,8-diazabicyclo[3.2.1]octane-8-carboxylate (Int-101e)

3.8-dichloro-5-(2,4-dimethoxybenzyl)pyrimido[5,4-c]pyridazin-6(5H)-one (Int-101d) (assumed 964 mg, 2.87 mmol) was dissolved in acetonitrile (10.0 mL), cooled to 0° C. (ice bath) and treated with N,N-diisopropylethylamine (2.50 mL, 14.3 mmol) and solid tert-butyl-(1S,5R)-1-methyl-3,8-diazabicyclo[3.2.1]octane-8-carboxylate (Int-II1-2) (973 mg. 4.30 mmol). The ice bath was removed, and the mixture stirred at ambient temperature for 1 h and then concentrated in vacuo. The residue was purified by column chromatography on silica gel (0 to 100% EtOAc/hexanes) to afford tert-butyl (1S,5R)-3-(3-chloro-5-(2,4-dimethoxybenzyl)-6-oxo-5,6-dihydropyrimido[5,4-c]pyridazin-8-yl)-1-methyl-3,8-diazabicyclo[3.2.1]octane-8-carboxylate (Int-101e), which was used without further purification. MS (ESI) [M+H]⁺ m/z 557/558.

### Step F: Tert-butyl (1S,5R)-3-(5-(2,4-dimethoxybenzyl-3-(3-hydroxynaphthalen-1-yl)-6-oxo-5,6-dihydropyrimido[5,4-c]pyridazin-8-yl)-1-methyl-3,8-diazabicyclo[3.2.1]octane-8-carboxylate (Int-101f)

A mixture of tert-butyl (1S,5R)-3-(3-chloro-5-(2,4-dimethylbenzyl)-6-oxo-5,6-dihydropyrimido[5,4-c]pyridazin-8-yl)-1-methyl-3,8-diazabicyclo[3.2.1]octane-8-carboxylate (Int-101e) (750 mg, 1.35 mmol), 4-(4,4,5,5-tetramethyl-1,3,2-dioxaborolan-2-yl)naphthalen-2-ol (453 mg, 1.68 mmol), sodium carbonate (474 mg. 4.47 mmol) and PdCl₂(dppf)CH₂Cl₂ (91 mg, 0.112 mmol) in 1.4-dioxane (5.0 mL) and water (1.25 mL) was subsurface sparged with nitrogen for 3 minutes. The vial was capped and heated at 95° C. for 3 h, cooled to ambient temperature and diluted with ethyl acetate (10 mL). The mixture was filtered through a stratified pad of anhydrous sodium sulfate over FLORISIL and CELITE and the filter pad rinsed with ethyl acetate (3×20 mL). The combined filtrate and rinses were concentrated. The residue was purified by column chromatography on silica gel (0 to 100%[1:3 EtOH/EtOAc]/hexanes) to afford tert-butyl (1 S,5R)-3-(5-(2,4-dimethylbenzyl)-3-(3-hydroxynaphthalen-1-yl)-6-oxo-5,6-dihydropyrimido[5,4-c]pyridazin-8-yl)-1-methyl-3,8-diazabicyclo[3.2.1]octane-8-carboxylate (Int-101f). MS (ESI) [M+H]⁺ m/z 665.

### Step G: Tert-butyl (1S,5R)-3-(3-(3-(((benzyloxy)carbonyl)oxy)naphthalen-1-yl)-5-(2,4-dimethoxybenzyl)-6-oxo-5,6-dihydropyrimido[5,4-c]pyridazin-8-yl)-1-methyl-3,8-diazabicyclo[3.2.1]octane-8-carboxylate (Int-101g)

Tert-butyl (1S,5R)-3-(5-(2,4-dimethoxybenzyl)-3-(3-hydroxynaphthalen-1-yl)-6-oxo-5,6-dihydropyrimido[5,4-c]pyridazin-8-yl)-1-methyl-3,8-diazabicyclo[3.2.1]octane-8-carboxylate (Int-101f) (700 mg, 1.05 mmol) in DCM (10 mL) was treated at room temperature with triethylamine (0.44 mL, 3.16 mmol) and Cbz-Cl (0.30 mL, 2.11 mmol). The mixture was stirred at ambient temperature for 18 h and then applied directly to a silica gel column. Purification by column chromatography on silica gel (0 to 100% [1:3 EtOH/EtOAc]/hexanes) afforded tert-butyl (1S,5R)-3-(3-(3-(((benzyloxy)carbonyl)oxy)naphthalen-1-yl)-5-(2,4-dimethoxybenzyl)-6-oxo-5,6-dihydropyrimido[5,4-c]pyridazin-8-yl)-1-methyl-3,8-diazabicyclo[3.2.1]octane-8-carboxylate (Int-101g). MS (EST) [M+H]⁺ m/z 799.

### Step H: Benzyl (4-(8-((1S,5R)-1-methyl-3,8-diazabicyclo[3.2.1]octan-3-yl)-6-oxo-5,6-dihydropyrimido[5,4-c]pyridazin-3-yl)naphthalen-2-yl) carbonate (Int-101h)

Tert-butyl(1S,5R)-3-(343-(((benzyloxy)carbonyl)oxy)naphthalen-1-yl)-5-(2,4-dimethoxybenzyl)-6-oxo-5,6-dihydropyrimido[5,4-c]pyridazin-8-yl)-1-methyl-3,8-diazabicyclo[3.2.1]octane-8-carboxylate (Int-101g) (670 mg, 0.84 mmol) was stirred in trifluoroacetic acid (10 mL) at 35° C. for 2 h and then cooled to ambient temperature and concentrated in vacuo. The residue was dissolved in DCM (20 mL) and concentrated in vacuo and then dried by vacuum for 30 minutes to afford benzyl (4-(8-((1S,5R)-1-methyl-3,8-diazabicyclo[3.2.1]octan-3-yl)-6-oxo-5,6-dihydropyrimido[5,4-c]pyridazin-3-yl)naphthalen-2-yl) carbonate (Int-101h), which was used without further purification. MS (ESI) [M+H]⁺ m/z 549.

### Step 1: Benzyl (1S,5R)-3-(3(3-(((benzyloxy)carbonyl)oxy)naphthalen-1-yl)-6-oxo-5,6-dihydropyrimido[5,4-c]pyridazin-8-yl)-1-methyl-3,8-diazabicyclo[3.2.1]octane-8-carboxylate (Int-1011)

Benzyl (4-(8-((1S,5R)-1-methyl-3,8-diazabicyclo[3.2.1]octan-3-yl)-6-oxo-5,6-dihydropyrimido[5,4-c]pyridazin-3-yl)naphthalen-2-yl) carbonate (Int-101h) (assumed 460 mg, 0.84 mmol), was dissolved in DCM (10.00 mL) and treated with N,N-diisopropylethylamine (0.73 mL, 4.19 mmol), cooled to 0° C. and treated with Cbz-CI (0.14 mL, 1.01 mmol) and stirred at 0° C. for 15 minutes. The ice bath was removed and the mixture was stirred at ambient temperature for 15 minutes. The reaction mixture was directly applied to a silica gel column and purified by column chromatography on silica gel (0 to 100% [1:3 EtOH/EtOAc]/hexanes) to afford benzyl (1S,5R)-3-(3-(3-(((benzyloxy)carbonyl)oxy)naphthalen-1-yl)-6-oxo-5,6-dihydropyrimido[5,4-c]pyridazin-8-yl)-1-methyl-3,8-diazabicyclo[3.2.1]octane-8-carboxylate (Int-1011). MS (ESI) [M+H]⁺ m/z 683.

### Step J: Benzyl (1S,5R)-3-(3-(3-(((benzyloxy)carbonyl)oxy)naphthalen-1-yl)-6-chloropyrimido[5,4-c]pyridazin-8-yl)-1-methyl-3,8-diazabicyclo[3.2.1]octane-8-carboxylate (Int-1011)

Benzyl (1S,5R)-3-(3-(3-(((benzyloxy)carbonyl)oxy)naphthalen-1-yl)-6-oxo-5,6-dihydropyrimido[5,4-c]pyridazin-8-yl)-1-methyl-3,8-diazabicyclo[3.2.1]octane-8-carboxylate (Int-101i) (150 mg, 0.22 mmol) in POCl₃(1000 μl, 10.7 mmol) was heated at 65° C. for 30 minutes, cooled to ambient temperature and concentrated. The residue was dissolved in DCM (2 mL) and treated with triethylamine (0.5 mL). The mixture was directly applied to a silica gel column. Purification by column chromatography on silica gel (0 to 100% [1:3 EtOH/EtOAc]/hexanes) afforded benzyl (1S,5R)-3-(3-(3-(((benzyloxy)carbonyl)oxy)naphthalen-1-yl)-6-chloropyrimido[5,4-c]pyridazin-8-yl)-1-methyl-3,8-diazabicyclo[3.2.1]octane-8-carboxylate (Int-101j), which was used without further purification. MS (ESI) [M+H]⁺ m¹z 701.

### Step K: Benzyl (1S,5R)-3-(6-((1-((dimethylamino)methyl)cyclopropyl)methoxy)-3-(3-hydroxynaphthalen-1-yl)pyrimido[5,4-c]pyridazin-8-yl)- 1-methyl-3,8-diazabicyclo[3.2.1]octane-8-carboxylate (Int-101k)

A vial containing benzyl (1S,5R)-3-(3-(3-(((benzyloxy)carbonyl)oxy)naphthalen-1-yl)-6-chloropyrimido[5,4-c]pyridazin-8-yl)-1-methyl-3,8-diazabicyclo[3.2.1]octane-8-carboxylate (Int-101j) (78.9 mg, 0.11 mmol) was charged with (1-[(dimethylamino)methyl]cyclopropyl)methanol (145 mg, 1.13 mmol), cesium fluoride (171 mg, 1.13 mmol) and DMSO (1.0 mL). The mixture was heated at 90° C. for 30 minutes, cooled to ambient temperature and filtered. The filtrate was purified by preparative RP-HPLC (MeCN/water with 0.1% TFA modifier) to afford benzyl (1S,5R)-346-((I-((dimethylamino)methyl)cyclopropyl)methyl)-3-(3-hydroxynaphthalen-1-yl)pyrimido[5,4-c]pyridazin-8-yl)-1-methyl-3,8-diazabicyclo[3.2.1]octane-8-carboxylate (Int-101k). MS (ESI) [M+H]⁺ a m/z 660.

### Step L: 4464(4(dimethylamino)methyl)cyclopropyl)methoxy)-8-((S,5R)-1-methyl-3,8-diazabicyclo[3.2.1]octan-3-yl)pyrimido[5,4-c]pyridazin-3-yl)naphthalen-2-ol (Ex. 101)

A mixture of benzyl (1S,5R)-3-(6-((1-((dimethylamino)methyl)cyclopropyl)methyl)-3-(3-hydroxynaphthalen-1-yl)pyrimido[5,4-c]pyridazin-8-yl)-1-methyl-3,8-diazabicyclo[3.2.1]octane-8-carboxylate (Int-101k) (30 mg, 0.039 mmol) and palladium(II) chloride (1,38 mg, 7.75 μmol) in DCM (0.5 mL) was treated at ambient temperature with triethylamine (0.016 mL, 0.116 mmol) followed by triethylsilane (0.093 mL, 0.58 mmol). The mixture was stirred at ambient temperature for 1 h, filtered with a syringe filter and the filtrate was concentrated. The residue was dissolved in tetrahydrofuran (1.0 mL) and treated with TBAF (I M in THF, 0.10 mL, 0.10 mmol). The mixture was stirred at ambient temperature for 1 h and then concentrated. The residue was purified by preparative RP-HPLC (MeCN/water with 0.1% TFA modifier) to afford 4-(6-((1-((dimethylamino)methyl)cyclopropyl)methoxy)-8-((1S,5R)-1-methyl-3,8-diazabicyclo[3.2.1]octan-3-yl)pyrimido[5,4-c]pyridazin-3-yl)naphthalen-2-ol (Ex. 101). MS (EST) [M+H]⁺ m/z 526, 1H NMR (499 MHz, Methanol-d₆) δ 7.94 (s, 1H), 7.81 (dd, J=18.0, 8.4 Hz, 2H), 7.50 (t, J=7.5 Hz, 1H), 7.36 (s, 2H), 7.32 (t, J=7.6 Hz, 1H), 4.50 (s, 2H), 4.39 (d, J=6.0 Hz, 1H), 3.35 (s, 2H), 3.03 (s, 6H), 2.42-2.12 (m, 3H), 2.05-1.91 (m, 11H), 1.67 (s, 3H), 1.05-0.97 (m, 2H), 0.97-0.86 (m, 2H).

The following compounds were synthesized in a similar fashion using intermediates described in other sections in this patent.

| | | | | | SFC |
|---|---|---|---|---|---|
| | Starting | | | [M + H]⁺ | Purification |
| Ex. | Material | Structure | Compound Name | Found | Conditions |
| Ex. 89b | 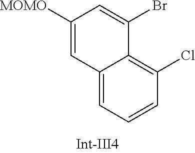 | 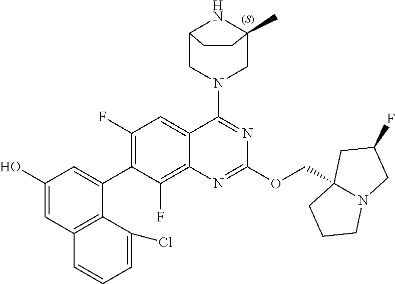 | tert-butyl (1S,5R)-3-(7-(8-chloro-3-methoxymethoxy) naphthalen-1-yl)-6,8-difluoro-2-(((2R,7aS)-2- fluorotetrahydro-1H-pyrrolizin-7a(5H)- yl)methoxy)quina- zolin-4-yl)-1-methyl-3,8-diazabicyclo[3.2.1]octane-8- carboxylate | 768 | Column: Chiralpak AD-3 150ᵢ Á 4.6 mm I.D., 3 um condition: A: CO2 B: iso- propanol (0.05% DEA) |
| | | Peak 2 from SFC | | | |
| Ex. 225 | S-pyrrolidine | 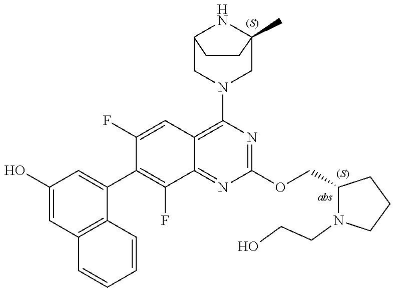 | 4-(6,8-difluoro-2-(((S)-1-(2-hydroxyethyl)pyrrolidin-2- yl)methoxy)-4-((1S,5R)-1-methyl-3,8-diazabicyclo [3.2.1]octan-3-yl)quinazolin-7-yl)naphthalen-2-ol | 576 | |
| Ex. 226 | 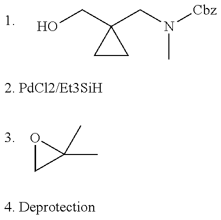 | 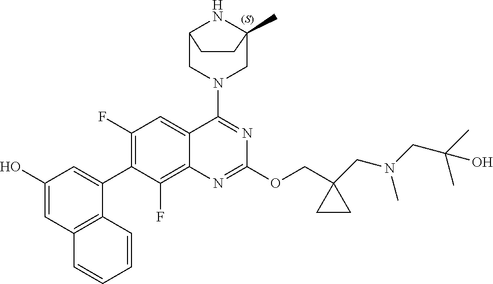 | 4-(6,8-difluoro-2-((1-(((2-hydroxy-2- methylpropyl)(methyl)amino)methyl)cyclopropyl)meth oxy)-4-((1S,5R)-1-methyl-3,8-diazabicy- clo[3.2.1]octan-3-yl)quinazolin-7-yl)naphthalen-2-ol | 618 | |

### Ex 227: Synthesis of 4-((1R,5S)_{3,8}-diazabicyclo[3.2.1]octan-3-y)-6,8-difluoro-2-(((2R,7aS)₂-fluorotetrahydro-1H-pyrrolizin-7a(5H)-yl)methoxy)quinazolin-7-yl)-6-methyl-5-(trifluoromethyl)-1H-indazole-3-carbonitrile- Peak 1 and 4-(4-((1R,5S)-3,8-diazabicyclo[3.2.1]octan-3-yl-6,8-difluoro-2-(((2R,7aS)-2-fluorotetrahydro-1H-pyrrolizin-7a(5H)-yl)methoxy)quinazolin-7-yl)-methyl-5-(trifluoromethyl)-1H-indazole-3-carbonitrile- Peak 2 (Ex. 227a and 227b)

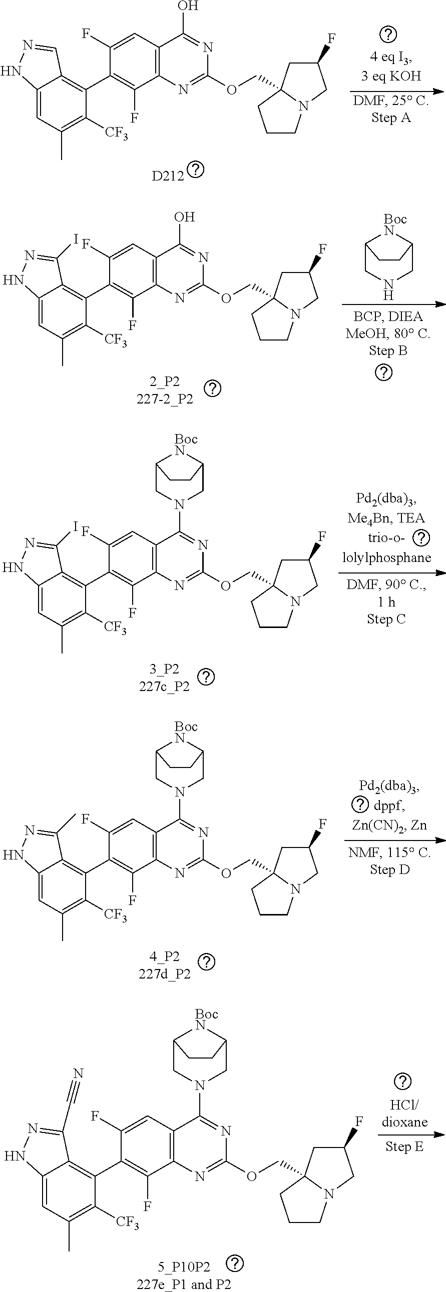

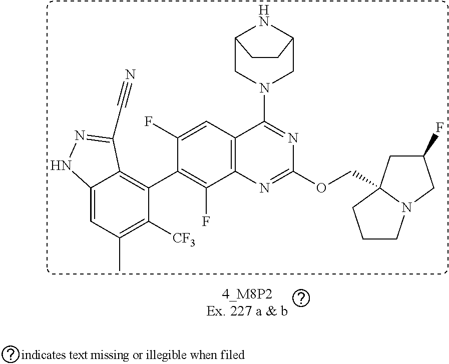 [SMILES: Cc1cc2[nH]nc(C#N)c2c(-c2c(F)cc3c(N4CC5CCC(C4)N5)nc(OC[C@@]45CCCN4C[C@H](F)C5)nc3c2F)c1C(F)(F)F]

### Step A: 6.8-difluoro-2-(((2R,7aS)-2-fluorotetrahydro-1H-pyrrolizin-7a(5H)-yl)methoxy)-7-(3-iodo-6-methyl-5-(trifluoromethyl)-1H-indazol-4-yl)quinazolin-4-ol (Int-227-2 P2)

To a solution of 6.8-difluoro-2-(((R,7aS)-2fluorotetrahydro-1H-pyrrolizin-7a(5H)-yl)methoxy)-7-(6-methyl-5-(trifluoromethyl)-1H-indazol-4-yl)quinazolin-4-ol (Int-140b_P2) (300 mg. 0.558 mmol) in DMF (5 mL) was added KOH (94 mg, 1.675 mmol) and 1-(567 mg, 2.233 mmol) at 25° C. The reaction was stirred at 25° C. for 2 h and monitored by LCMS, The reaction mixture was quenched with aqueous Na₂SO₃ solution (2 M, 20 mL), extracted with EtOAc (20 mL×3). The combined organic phase was dried over anhydrous Na₂SO₄, filtered and concentrated in vacuo to give 6.8-difluoro-2-(((2R,7aS)-2-fluorotetrahydro-1H-pyrrolizin-7a(5H)-yl)methoxy)-7-(3-iodo-6-methyl-5-(trifluoromethyl)-1H-indazol-4-yl)quinazolin-4-ol which was used in the next step directly. MS (ESI): m/z (M+H)⁺ 664.

### Step B: Tert-butyl (1R,5S)-3-(6,8-difluoro-2-(((2R,7aS)-2-fluorotetrahydro-1H-pyrrolizin-7a(5H)-yl)methoxy)-743-iodo-6-methyl-5-(trifluoromethyl)-1H-indazol-4-yl)quinazolin-4-yl)-3,8-diazabicyclo[3.2.1]octane-8-carboxylate (Int-227c_P2)

To a solution of 6.8-difluoro-2-(((2R,7aS)-2-fluorotetrahydro-1H-pyrrolizin-7a(5H)-yl)methoxy)-7-(3-iodo-6-methyl-5-(trifluoromethyl)-1H-indazol-4-yl)quinazolin-4-ol (Int-227-2_P2) (200 mg, 0.301 mmol) in MCCN (0.5 mL) was added BOP (200 mg, 0.452 mmol) at 25° C. The mixture was stirred at 25° C. for 30 min, and then tert-butyl (1R,5S)-3,8-diazabicyclo[3.2.1]octane-8-carboxylate (128 mg, 0.603 mmol) and DIEA (0.158 mL, 0.904 mmol) was added at 25° C. The reaction mixture was stirred at 80° C. for 1 h and monitored by LCMS, The solvent was evaporated under reduced pressure to give the crude which was purified by preparative Prep-TLC (EtOAc) to give tert-butyl (1R,5S)-3-(6,8-difluoro-2-(((2R,7aS)-2-fluorotetrahydro-1H-pyrrolizin-7a(5H)-yl)methoxy)-7-(3-iodo-6-methyl-5-(trifluoromethyl)-1H-indazol-4-yl)quinazolin-4-yl)-3,8-diazabicyclo[3.2.1]octane-8-carboxylate. MS (ESI): m/z (M+H)⁺ 858.

### Step C: Tert-butyl (1R,5S)-3-(7-(3.6-dimethyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-227d P2)

To a mixture of tert-butyl (1R,5S)-3-(6,8-difluoro-2-(((2R,7aS)-2-fluorotetrahydro-1H-pyrrolizin-7a(5H)-yl)methoxy)-7-(3-iodo-6-methyl-5-(trifluoromethyl)-1H-indazol-4-yl)quinazolin-4-yl)-3,8-diazabicyclo[3.2.1]octane-8-carboxylate (Int-227c_P2) (40 mg, 0.047 mmol) in DMF (1 mL) was added tris-o-tolylphosphine (2.84 mg, 9.33 μmol), tris(dibenzyldeneacetone)dipalladium(0) (4.27 mg, 4.66 μmol), tetramethylstannane (25.02 mg, 0.140 mmol) and triethylamine (14.63 mg, 0.145 mmol). The reaction mixture was stirred at 80° C. for 1 h under nitrogen atmosphere and monitored by LCMS, The reaction mixture was quenched with saturated aqueous water (5 mL) and extracted with EtOAc (3×10 mL). The combined organic layers were washed with brine (3×10 mL), dried over Na₂SO₄, filtered and the solvent was evaporated under reduced pressure to give the crude which was purified by preparative Prep-TLC (EtOAc) to give tert-butyl (1R,5S)-3-(7-(3.6-dimethyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI): m/z (M+H)⁺ 746.

### Step D: Tert-butyl (1R,5S)-3-(7-(3-cyano-6-methyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-227e_P1 and Int-227e_P2)

To a mixture of tert-butyl (1R,5S)-3-(7-(3.6-dimethyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-227_P2) (60 mg, 0.070 mmol) in NMP (1 mL) was added tris(dibenzyldeneacetone)dipalladium(0) (6.41 mg, 7.00 μmol), dppf (7.76 mg, 0.014 mmol), zinc cyanide (41.1 mg, 0.350 mmol) and zinc (9.15 mg, 0.140 mmol). The reaction was stirred at 110° C. under N₂ for 2 h. The reaction mixture was quenched with water (3 mL), extracted with EtOAc (3×3 mL). The organic layers were washed with saturated brine (3×3 mL), dried over Na₂SO₄, filtered and concentrated in vacuum, the residue was purified by prep-TLC (EtOAc) to give tert-butyl (1R,5S)-3-(7-(3-cyano-6-methyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI), m/z (M+H)⁺ 757.

### Step E: 4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-methyl-5-(trifluoromethyl)-1H-indazole-3-carbonitrile (Ex. 227a-Peak 1)

A solution of tert-butyl (1R,5S)-3-(7-(3-cyano-6-methyl-5-(trifluoromethyl)-1H-indazol-4-yl)-6,8-difluoro-2-(((2R,7aS)-2-fluorotetrahydro-1H-pyrrolizin-7a(5H)-yl)methoxy)quinazolin-4-yl-3,8-diazabicyclo[3.2.1]octane-8-carboxylate (Int-227e_P1) (30 mg, 0.040 mmol) in hydrogen chloride (4 M in Dioxane) (0.5 mL, 2.000 mmol) was stirred at 20° C. for 1 h. LCMS showed desired product mass was observed. The mixture was concentrated, and the residue was purified by prep-SFC (Instrument SFC-21 Method Column DAICEL CHIRALCEL OD-H (250 mm*30 mm, 5 um) Condition 0.1% NH₃H₂O EtOH Begin B 35% End B 35% Gradient Time (min) 100% B Hold Time (min) FlowRate (mL/min) 80 Injections 60) to give 4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-methyl-5-(trifluoromethyl)-1H-indazole-3-carbonitrile. MS (ESI): m/z (M+H)⁺ 657. Ex. 227a-Peak 1: ¹H NMR (400 MHz, McOD) δ 7.86 (s, 1H), 7.66 (d, J=10.0 Hz, 1H), 5.45-5.24 (m, 1H), 4.60 (d, J=13.0 Hz, 11H), 4.46-4.23 (m, 3H), 3.72 (d, J=11.5 Hz, 3H), 3.64-3.53 (m, 1H), 3.48-3.32 (m, 2H), 3.17-3.01 (m, 1H), 2.73 (s, 3H), 2.52-2.14 (m, 4H), 2.13-1.80 (m, 7H). The procedures of Int-227e_P2 was similar as Int-227e_P1. The spectrum is as below: MS (ESI): m/z (M+H)⁺ 657. Ex.227b-Peak2: ¹H NMR (400 MHz, MeOD) δ 7.86 (s, 1H), 7.66 (d, J=10.0 Hz, 1H), 5.45-5.24 (m, 1H), 4.60 (d, J=13.0 Hz, 1H), 4.46-4.23 (m, 3H), 3.72 (d, J=11.5 Hz, 3H), 3.64-3.53 (m, 1H), 3.48-3.32 (m, 2H), 3.08 (dt, J=6.0, 9.5 Hz, 1H), 2.73 (s, 3H), 2.52-2.14 (m, 4H), 2.13-1.80 (m, 7H).

### Ex. 228: Synthesis of 6-(4-((1R,5S)-3,8-diazabicyclo[3.2.1]octan-3-v-6-chloro-8-fluoro-2-(((2R,7aS)-2-fluorotetrahydro-1H-pyrrolizin-7a(5H)-yl)methoxy)quinazolin-7-yl-4-methyl-5-(trifluoromethyl)pyridin-2-amine

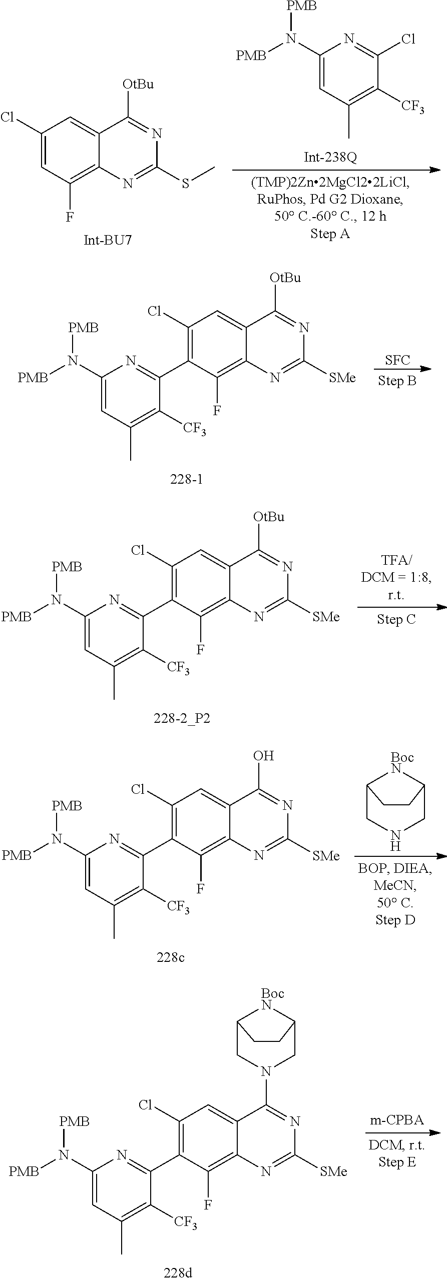

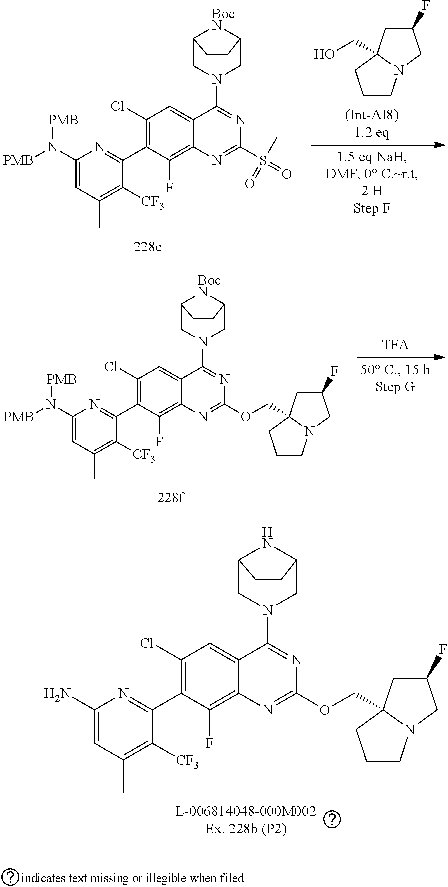

### Step A: 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-1)

A solution of 4-(tert-butoxy)-6-chloro-8-fluoro-2-(methylthio)quinazoline (Int-BU7) (32 g, 106 mmol) in (TMP)₂ Zn-2MgCl₂·LiCl (532 mL, 213 mmol) (0.4 M in THF) was stirred at 50° C. for 3 h. After cooled to room temperature, chloro(2-dicyclohexylphosphino-2′,6′-diisopropoxy-1,1′-biphenyl)[2-(2′-amino-1,1′-biphenyl)]palladium(II) (12.40 g, 15.% mmol) and 6-chloro-N,N-bis(4-methoxybenzyl)-4-methyl-5-(trifluoromethyl)pyridin-2-amine (Int-236q) (48.0 g, 106 mmol) in THF (200 mL) was added to the reaction solution in glove box, and the mixture was stirred at 60° C. for 12 h. The reaction mixture was quenched with sat. aqueous NaHCO₃(300 mL), extracted with EtOAc (3×500 mL), washed with brine, and dried over NaSO₄. The organic layer was filtered and the filtrate was concentrated under reduced pressure, and the residue was purified by flash silica gel chromatography 220 g column, Eluent of 0˜18% EtOAc/Pet, ether gradient to provide 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine. MS (EST) m/z:. (M+H)⁺ 715.

### Step B: 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-2 P1 and Int-228-2 P2)

The racemic 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-1) (42 g, 58.7 mmol) was separated by preparative SFC (Column: DAICEL CHIRALPAK AS (250 mm*50 mm, 10 um) Condition 0.1% NH₃H₂O IPA Begin B 30 End B 30 Gradient Time (min) 100 100% B Hold Time 100 Flow Rate (mL/min) 200 Injections 900) to give 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-2_P1) the first eluting isomer, 100% ee) as yellow solid and 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-2_P2), the second eluting isomer, 100% ee). Int-228-2_P2: ¹H NMR (400 MHz. CDCl₃) δ 7.80 (d, J=1.2 Hz, 1H), 7.07 (d, J=8.6 Hz, 4H), 6.77 (d, J=8.6 Hz, 4H), 6.32 (s, 1H), 4.69 (br d, J 15.7 Hz, 2H), 4.44 (d, J=16.0 Hz, 2H), 3.72 (s, 6H), 2.57 (s, 3H), 2.34 (s, 3H), 1.66 (s, 9H). MS (ESI): m/z (M+H)⁺ 715.

Peak 2 was carried forward through the next steps described below.

### Step C: 7-(6-(bis(4-methoxybenzyl)amino)-4-methyl-3-(trifluoromethyl)pyridin-2-yl)-6-chloro-8-fluoro-2-(methylthio)quinazolin-4-ol (Int-228c)

To a solution of 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-228-2_P2) (2 g, 2.80 mmol) in DCM (20 mL) was added TFA (1 mL, 12.98 mmol) at room temperature (20° C.). The mixture was stirred at 20° C. for 3 h. The mixture was diluted with DCM (100 mL) and H₂O (20 mL), basified with sodium hydroxide aqueous solution (2 M) until pH 7. The organic phase was separated and dried with Na₂SO₄, filtered and the solvent was evaporated under reduced pressure to give the crude product 7-(6-(bis(4-methoxybenzyl)amino)-4-methyl-3-(trifluoromethyl)pyridin-2-yl)-6-chloro-8-fluoro-2-(methylthio)quinazolin-4-ol, which was used to next step directly without purification. MS (ESI): m/z (M+H)⁺ 659.

### Step D: Tert-butyl (1R,5S)-3-(7-(6-(bis(4-methoxybenzyl)amino)-4-methyl-3-(trifluoromethyl)pyridin-2-yl)-6-chloro-8-fluoro-2-(methylthio)quinazolin-4-yl)-3,8-diazabicyclo[3.2.1]octane-8-carboxylate (Int-228d)

To a solution of 7-(6-(bis(4-methoxybenzyl)amino)-4-methyl-3-(trifluoromethyl)pyridin-2-yl)-6-chloro-8-fluoro-2-(methylthio)quinazolin-4-ol (Int-228c) (1.843 g, 2.80 mmol) in MeCN (18 mL) was added BOP (1.855 g, 4.19 mmol), DIEA (1.465 mL, 8.39 mmol) and tert-butyl (1R,5S)-3,8-diazabicyclo[3.2.1]octane-8-carboxylate (0.712 g, 3.36 mmol) at room temperature (15° C.). The mixture was stirred at 50° C. for 1 h. TLC (SiO₂; petroleum ether: ethyl acetate=3:1) showed starting material was consumed and a new spot was observed. The mixture was cooled and the solvent was evaporated under reduced pressure to give the crude product. The crude product was purified by flash silica gel chromatography 20 g Column, Eluent of 15% ethyl acetate in petroleum ether gradient to give tert-butyl (1R,5S)-3-(7-(6-(bis(4-methoxy benzyl)amino)-4-methyl-3-(trifluoromethyl)pyridin-2-yl)-6-chloro-8-fluoro-2-(methylthio)quinazolin-4-yl)-3,8-diazabicyclo[3.2.1]octane-8-carboxylate. MS (ESI): m z (M+H)⁺ 853. ¹H NMR (400 MHz, MeOD) δ 7.82 (s, 1H), 7.13 (br d, J=7.2 Hz. 4H), 6.83 (br d, J=6.9 Hz, 4H), 6.61 (s, 1H), 4.77 (br d, J=15.5 Hz, 2H), 4.62 (s, 1H), 4.48-4.60 (m, 2H), 4.28-4.44 (m, 3H), 3.76 (d, J=1.9 Hz, 6H), 3.66 (br d, J=12.0 Hz, 1H), 3.56 (br d, J=12.6 Hz, 1H), 2.59 (s, 3H), 2.41 (br s, 3H), 1.80-1.95 (m, 3H). 1.74 (br s, 1H), 1.52 (s, 9H).

### Step E: Tert-butyl (1R,5S)-3-(7-(6-(bis(4-methoxybenzyl)amino)-4-methyl-3-(trifluoromethyl)pyridin-2-yl)-6-chloro-8-fluoro-2-(methylsulfonyl)quinazolin-4-yl)-3,8-diazabicyclo[3.2.1]octane-8-carboxylate (Int 228e)

To a solution of tert-butyl (1R,5S)-3-(7-(6-(bis(4-methoxybenzyl)amino)-4-methyl-3-(trifluoromethyl)pyridin-2-yl)-6-chloro-8-fluoro-2-(methylthio)quinazolin-4-yl)-3,8-diazabicyclo[3.2.1]octane-8-carboxylate (Int-228d) (2.12 g, 2.484 mmol) in DCM (20 mL) was added m-CPBA (1.009 g, 4.97 mmol, 85% Wt) at 0° C. The mixture was stirred at room temperature (17° C.) for 1 h. TLC (SiO₂; petroleum ether: ethyl acetate=2:1) showed starting material was consumed and a new spot was formed. The mixture was cooled, diluted with DCM (100 mL), quenched with sodium bicarbonate aqueous solution (30 mL, 2 M), washed with saturated sodium sulfite aqueous solution (15 mL), dried over Na₂SO₄, filtered and the solvent was evaporated under reduced pressure. The crude product was purified by flash silica gel chromatography 20 g column, Eluent of 15% ethyl acetate in petroleum ether gradient to give tert-butyl (1R,5S)-3-(7-(6-(bis(4-methoxybenzyl)amino)-4-methyl-3-(trifluoromethyl)pyridin-2-yl)-6-chloro-8-fluoro-2-(methylsulfonyl)quinazolin-4-yl)-3,8-diazabicyclo[3.2.1]octane-8-carboxylate. MS (EST): m/z (M+H)⁺ 885. Int-228e_P2: ¹H NMR (500 MHz, MeOD) δ 8.04 (s, 1H), 7.13 (d, J=8.5 Hz, 4H), 6.83 (d, J=8.5 Hz, 4H), 6.65 (s, 1H), 4.77 (br d, J=15.7 Hz, 2H), 4.71 (br d, J=13.0 Hz, 1H), 4.60 (d, J=15.7 Hz, 2H), 4.54 (br d, J=12.7 Hz, 1H), 4.32-4.41 (m, 2H), 3.80 (br d, J=12.4 Hz, 1H), 3.76 (s, 6H), 3.69 (br d, J=13.0 Hz, 1H), 3.39 (s, 3H), 2.42 (s, 3H), 1.77-1.95 (m, 3H), 1.65-1.75 (m, 1H), 1.53 (s, 9H).

### Step F: Tert-butyl (0R,5S)-3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-228f)

To a solution of((2R,7aS)-2-fluorotetrahydro-1H-pyrrolizin-7a(5H)-yl)methanol (Int-A18) (144 mg, 0.904 mmol) in THF (5 mL) was added NaH (36.1 mg, 0.904 mmol) (60% in mineral oil) at 0° C. under N₂. The mixture was stirred at 0° C. for 10 min, then tert-butyl (1R,5S)-3-(7-(6-(bis(4-methoxybenzyl)amino)-4-methyl-3-(trifluoromethyl)pyridin-2-yl)-6-chloro-8-fluoro-2-(methylsulfonyl)quinazolin-4-yl)-3,8-diazabicyclo[3.2.1]octane-8-carboxylate (Int-228d) (400 mg, 0.452 mmol) in THF (1 mL) was added to the above mixture, and the resulting mixture was stirred at 0° C. for 2 h. The reaction mixture was quenched with water (2 mL) and extracted with EtOAc (3×30 mL). The organic layer was dried over Na₂SO₄, filtered and the solvent was evaporated under reduced pressure to give crude product tert-butyl (1R,5S)-3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate as brown oil, which was used for the next step without further purification. MS (ESI) m/z: (M+H)⁺ 964.

### Step G: 6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine (Ex. 228b) (P2)

Tert-butyl (1R,5S)-3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-228f) (430 mg, 0.446 mmol) was added to TFA (5 mL). The reaction mixture was stirred at 50° C. for 15 h. The reaction mixture was concentrated in vacuo to give the crude product, which was diluted MeOH (2 mL) and basified with sat Na₂CO₃ to pH 8. The mixture was filtered and the filtrate was purified by pre-HPLC (Column. Waters Xbridge BEH C18 100×40×10 um, Condition: Begin B 36 End B 66 Gradient Time (min) 11 100% B Hold Time 4 Flow Rate (mL/min) 50 Injections 5) to give 6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine. MS (ESI): m/z (M+H)⁺ 624. Ex. 228b: ¹H NMR (400 MHz, MeOD) 7.74 (d, J=1.3 Hz, 1H), 6.50 (s, 1H), 5.33-5.03 (m, 1H), 4.35 (br t, J=11.4 Hz. 2H). 4.21-4.14 (m, 1H), 4.13-4.04 (m, 1H). 3.58-3.44 (m, 4H), 3.20-3.03 (m, 3H), 2.91 (dt, J=5.5, 9.3 Hz, 1H), 2.35 (d, J=1.3 Hz, 3H), 2.29-1.99 (m, 3H), 1.94-1.62 (m, 7H).

### Ex 229: Synthesis of 4-(6,8-difluoro-2-(((2R7aS -2-fluorotetrahydro-1H-pyrrolizin-7a(5H)-yl)methoxy)-4-((S5)-1-methyl-38-diazabicyclo[3.2.1]octan-3-yl)quinazolin-7-yl)-3-ethylpyrazolo[1.5-a]pyridin-6-ol

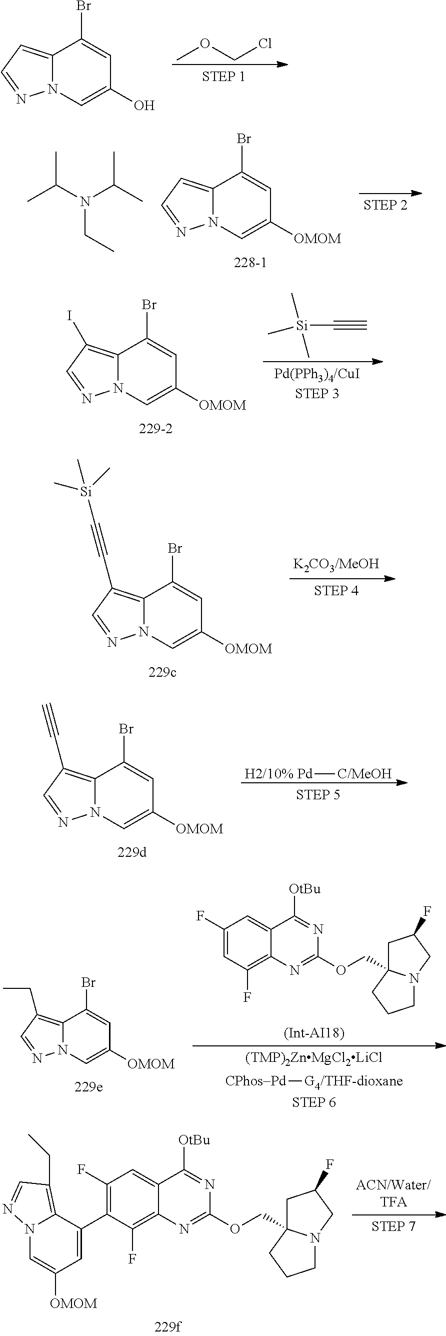

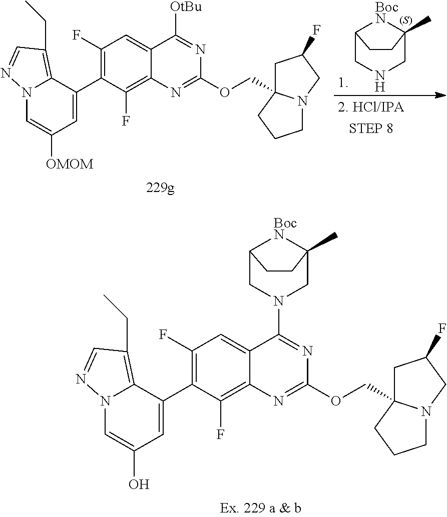

### STEP 1: 4-bromo-6-(methoxymethoxy)pyrazolo[1.5-a]pyridine (Int-229-1)

4-bromopyrazolo[1.5-a]pyridin-6-ol (500.0 mg, 2.347 mmol) was dissolved in DCM (10.00 ml) and cooled in ice-bath. Chloro(methoxy)methane (0.378 ml, 4.69 mmol) and N-ethyl-N-isopropylpropan-2-amine (1.671 ml, 9.39 mmol) were added and the reaction mixture was warmed to rt and stirred for 16 hrs. The reaction mixture was diluted with ethyl acetate. The organic layer was washed with saturated aq. NaHCO₃ solution. The organic layer was separated and dried over MgSO₄, filtered and excess solvent was removed under reduced pressure. The residue obtained was purified on a 40-g silica gel column, using gradient elution with Hex- Ethyl acetate (0-80%.) Appropriate fractions were pooled together, excess solvent was removed under reduced pressure and the oil obtained was vacuum dried to give 4-bromo-6-(methoxymethoxy)pyrazolo[1.5-a]pyridine. MS (ESI): m/z (M+H)⁺ 257/259.

### STEP 2: 4-bromo-3-iodo-6-(methoxymethoxy)pyrazolo[1.5-a]pyridine (Int-229-2)

4-bromo-6-(methoxymethoxy)pyrazolo[1.5-a]pyridine (Int-229-1) (603.0 mg, 2.346 mmol) was dissolved in Acetonitrile (15.00 ml) and cooled in ice-bath. N₁S (1055 mg, 4.69 mmol) was added in small portions and the reaction mixture was warmed to rt and stirred for 16 hrs. The reaction mixture was diluted with ethyl acetate. The organic layer was washed with saturated sodium bicarbonate aqueous solution. The organic layer was separated and dried over MgSO₄, filtered and excess solvent was removed under reduced pressure. The residue obtained was purified on a 40-g silica gel column, using gradient elution with Hex- Ethyl acetate/ethanol 3:1 (v/v) mix. 0-70%. Appropriate fractions were pooled together, excess solvent was removed under reduced pressure and the oil obtained was vacuum dried to give 4-bromo-3-iodo-6-(methoxymethoxy)pyrazolo[1.5-a]pyridine. MS (ESI): m/z (M+H)⁺ 383/385.

### STEP 3: 4-bromo-6-(methoxymethoxy)-3-((trimethylsilyl)ethynyl)pyrazolo[1.5-a]pyridine (Int-229c)

To a vial were added 4-bromo-3-iodo-6-(methoxymethoxy)pyrazolo[1.5-a]pyridine (Int-229-2) (200 mg, 0.522 mmol), Trimethylsilylacetylene (0.220 ml, 1.567 mmol), tetrakis(triphenylphosphine)palladium(0) (60.3 mg, 0.052 mmol), Copper(I) iodide (59.7 mg, 0.313 mmol) in DMF (4 ml). To this TEA (0.146 ml, 1.044 mmol) was added and stirred for 30 min. The reaction mixture was diluted in ethyl acetate, washed with saturated aqueous sodium hydrogen carbonate and brine then dried over Na₂SO₄. The reaction was filtered and concentrated under reduced pressure. The residue was purified on silica gel column using gradient elution with (0-25%) Hex-Ethyl acetate. 4-bromo-6-(methoxymethoxy)-3-((trimethylsilyl)ethynyl)pyrazolo[1.5-a]pyridine was obtained as a mixture. MS (ESI): m/z (M+H)⁺ 353.

### STEP 4: 4-bromo-3-ethynyl-6-(methoxymethoxypyrazolo[1.5-a]pyridine (Int-229d)

To 4-bromo-6-(methoxymethoxy)-3-((trimethylsilyl)ethynyl)pyrazolo[1.5-a]pyridine (Int-229c) (184.0 mg, 0.521 mmol) in MeOH (4.00 ml), K₂CO₃ (180 mg, 1.302 mmol) was added and stirred for 30 min at rt. The reaction mixture was diluted in ethyl acetate, washed with saturated NaHCO₃ solution and brine then dried over MgSO₄, filtered and concentrated. The residue was purified on a silica gel column (40 gm ISCO column) using gradient elution with ethyl acetate/Hex (0-35%). 4-bromo-3-ethynyl-6-(methoxymethoxy)pyrazolo[1.5-a]pyridine was carried forward as a mixture. MS (ESI): m/z (M+H)⁺ 282.

### STEP 5: 4-bromo-3-ethyl-6-(methoxymethoxy)pyrazolo[1.5-a]pyridine (Int-

To a flask fitted with a three way key, one attached to a N₂ and the other to vacuum, 4-bromo-3-ethynyl-6-(methoxymethoxy)pyrazolo[1.5-a]pyridine (Int-229d) (140.0 mg, 0.498 mmol) and MeOH (4.00 ml) were added. The flask was thoroughly purged with vacuum and N₂ and then under N₂, 10% Palladium on carbon (15.0 mg, 0.014 mmol) was added. The flask was again purged with N₂ and the N₂ line was switched to a H₂ balloon. The flask was alternately bleeded with H₂ and vacuum three times and then H₂ was allowed to flow freely for 2.5 hours. The reaction was monitored every half hour using LC-MS, The reaction mixture was filtered through a CELITE filter bed. The filter bed was washed with excess MeOH. The filtrate was concentrated under reduced pressure and vacuum dried to give 4-bromo-3-ethyl-6-(methoxymethoxy)pyrazolo[1.5-a]pyridine. This was used for the next step directly without purification. MS (ESI): m/z (M+H)⁺ 286.

### STEP 6: 4-(tert-butoxy)-7-(3-ethyl-6-(methoxymethoxy)pyrazolo[1.5-a]pyridin-4-yl)-6,8-difluoro-2-(((2R,7aS)-2-fluorotetrahydro-1H-pyrrolizin-7a(5H)-yl)methoxyquinazoline (Int-229f)

To (TMP)₂Zn·MgCl₂·LiCl (1.581 ml, 0.316 mmol) in a vial was added a solution of 4-(tert-butoxy)-6,8-difluoro-2-(((2R,7aS)-2-fluorotetrahydro-1H-pyrrolizin-7a(5H)-yl)methoxy)quinazoline (Int-AI10) (50 mg, 0.126 mmol) in THF (0.50 ml). The reaction was stirred at rt for two hours. To check for completion an aliquot was quenched with iodine. A solution of 4-bromo-3-ethyl-6-(methoxymethoxy)pyrazolo[1.5-a]pyridine (Int-229d) (60.0 mg, 0.210 mmol) and CPhos Pd G4 (10.37 mg, 0.013 mmol) in 1.4-Dioxane (1.0 ml), thoroughly purged with N₂ was added slowly at rt to the above reaction mixture and the reaction was stirred at 55° C. for 16 hours. The reaction was quenched with MeOH. 15 mL of sat. NaHCO₃ solution and 15 mL of EtOAc were added. The organic layer was separated, washed with brine, dried over MgSO₄, filtered and excess solvent was removed under reduced pressure. The oil obtained was purified on a silica gel column (40 gm) using gradient elution with DCM/MeOH (0-25%). Appropriate fractions were pooled together, excess solvent was removed under reduced pressure and vacuum dried to give 4-(tert-butoxy)-7-(3-ethyl-6-(methoxymethoxy)pyrazolo[1.5-a]pyridin-4-yl)-6,8-difluoro-2-(((2R,7aS)-2-fluorotetrahydro-1H-pyrrolizin-7a(5H)-yl)methoxy)quinazoline. MS (ESI): m/z (M+H)⁺ 600.

### STEP 7: 7-(3-ethyl-6-(methoxymethoxy)pyrazolo[1,5-a]npyridin-4-yl)-6,8-difluoro-2-(((2R,7aS)-2-fluorotetrahydro-1H-pyrrolizin-7a(5H)-yl)methoxy)quinazolin-4-ol (Int-229)

To 4-(tert-butoxy)-7-(3-ethyl-64(methoxymethoxy)pyrazolo[1.5-a]pyridin-4-yl)-6,8-difluoro-2-(((2R,7aS)-2-fluorotetrahydro-1H-pyrrolizin-7a(5H)-yl)methoxy)quinazoline (Int-229f) (70.0 mg, 0.117 mmol), Acetonitrile (1.0 ml) and Water (0.256 ml) and TFA (0.026 ml) were added and the reaction stirred at rt for 2 hours. The reaction was quenched with water. 15 mL of sat. NaHCO₃ solution and 15 mL of EtOAc were added. The organic layer was separated, washed with brine, dried over MgSO₄, filtered and excess solvent was removed under reduced pressure. The oil obtained was purified on a silica gel column (40 gm) using gradient elution with DCM/MeOH (0-25%). Appropriate fractions were pooled together, excess solvent was removed under reduced pressure and was vacuum dried to give 7-(3-ethyl-6-(methoxymethoxy)pyrazolo[1.5-a]pyridin-4-yl-6,8-difluoro-2-(((2R,7aS)-2-fluorotetrahydro-1H-pyrrolizin-7a(5H)-yl)methoxy)quinazolin-4-ol. MS (ESI): m/z (M+H)⁺ 544.

### STEP 8: 4-(6,8-difluoro-2-(((2R,7aS)-2-fluorotetrahydro-1H-pyrrolizin-7a(5H)-yl)methoxy)-4-((1S,5R)-1-methyl-38-diazabicyclo[3.2.1]octan-3-yl)quinazolin-7-yl)-3-ethylpyrazolo[1.5-a]pyridin-6-ol (Ex.229)

To 7-(3-ethyl-6-(methoxymethoxy)pyrazolo[1.5-a]pyridin-4-yl)-6,8-difluoro-2-(((2R,7aS)-2-fluorotetrahydro-1H-pyrrolizin-7a(5H)-yl)methoxy)quinazolin-4-ol (Int-229g) (18.00 mg, 0.033 mmol) and tert-butyl (1S,5R)-1-methyl-3,8-diazabicyclo[3.2.1]octane-8-carboxylate (Int-II1-2) (8.99 mg, 0.040 mmol) in ACN (0 ml), BOP ((1H-benzo[d][1.2,3]triazol-1-yl)oxy)tris(dimethylamino)phosphonium hexafluorophosphate(V) (29.3 mg, 0.066 mmol) and DIEA (0.029 ml, 0.166 mmol) were added and the reaction was stirred at 75° C. for 60 mins. Excess solvent was removed under reduced pressure and to the residue 1 mL isopropanol was added and HCl (4M HCl in dioxane soln) (0.166 ml, 0.662 mmol) was added and reaction was stirred at 45° C. for three hours. Excess solvent was removed under reduced pressure and the oil obtained was dissolved in MeOH, filtered, and directly purified on a reverse phase HPLC using gradient elution with 0-100% ACN-water using 0.1% TFA as a modifier. Appropriate fractions were pooled together and lyophylzed to give a racemic mixture. This was further resolved via SFC (conditions listed in table below) to give 4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-3-ethylpyrazolo[1.5-a]pyridin-6-ol as Peak 1-Ex.229-A, MS (EST): m/z (M+H)⁺ 608. ¹H NMR (600 MHz, DMSO-d₆) δ 8.20 (s, 1H), 7.72 (s, 2H), 6.96 (s, 1H), 5.28 (d, J=54.3 Hz, 1H), 4.43 (d, J=12.2 Hz, 1H), 4.24-4.06 (m, 2H), 4.06-3.95 (m, 1H), 3.75 (s, 1H), 3.51-3.47 (m, 2H), 3.11-2.99 (m, 4H), 2.85 (s, 1H), 2.18-2.10 (m, 1H), 2.11-2.03 (m, 3H), 2.02-1.99 (m, 1H), 1.88-1.83 (m, 2H), 1.79 (s, 2H), 1.48-1.41 (m, 1H), 1.31 (s, 3H), 1.25 (s, 1H), 0.85 (d, J=15.0 Hz, 4H) and 4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-3-ethylpyrazolo[1.5-a]pyridin-6-ol as Peak 2-Ex.229-B, MS (ESI): m/z (M+H)⁺ 608. ¹H NMR (600 MHz, DMSO-d₆) δ 8.19 (s, 1H), 7.71 (s, 2H), 6.96 (s, 1H), 5.27 (d, J=54.1 Hz, 1H), 4.27 (d, J=12.0 Hz, 1H), 4.18 (s, 1H), 4.05 (d, J=24.5 Hz, 2H), 3.51 (d, J=9.4 Hz, 2H), 3.17 (d, J=12.0 Hz, 2H), 3.05 (d, J=37.3 Hz, 4H), 2.91-2.78 (m, 1H), 2.15-2.01 (m, 4H), 1.88-1.75 (m, 4H), 1.60 (d, J=49.0 Hz, 1H), 1.33 (d, J=28.3 Hz. 2H), 1.23 (s, 3H) 0.92-0.81 (m, 3H).

| | | | [M + H]⁺ | SFC |
|---|---|---|---|---|
| Ex. | Structure | Compound Name | Found | Purification Conditions |
| Ex. 230a | 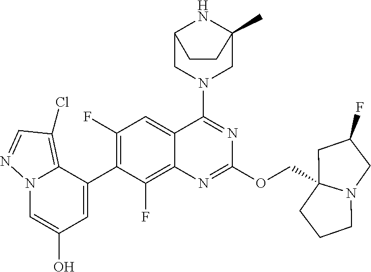 [SMILES: C[C@]12CCC(CN(c3nc(OC[C@@]45CCCN4C[C@H](F)C5)nc4c(F)c(-c5cc(O)cn6ncc(Cl)c56)c(F)cc34)C1)N2] | 3-chloro-4-(6,8-difluoro-2-(((2R,7aS)- 2-fluorotetrahydro-1H-pyrrolizin- 7a(5H)-yl)methoxy)-4-((1S, 5R)-1-methyl-3,8- diazabicyclo[3.2.1]octan-3- yl)quinazolin-7-yl)pyrazolo[1,5- a]pyridin-6-ol, TFA | 614 | Condition M, 30% IPA w/0.1% NH4OH |
| | Peak 1, SFC resolution after STEP 7 | | | |
| Ex. 230b | 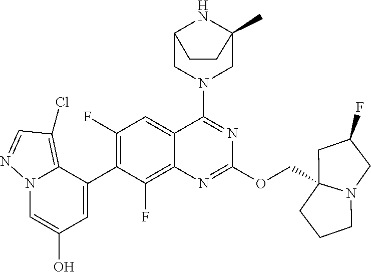 [SMILES: C[C@]12CCC(CN(c3nc(OC[C@@]45CCCN4C[C@H](F)C5)nc4c(F)c(-c5cc(O)cn6ncc(Cl)c56)c(F)cc34)C1)N2] | 3-chloro-4-(6,8-difluoro-2-(((2R,7aS)- 2-fluorotetrahydro-1H-pyrrolizin- 7a(5H)-yl)methoxy)-4-((1S,5R)-1- methyl-3,8-diazabicyclo[3.2.1]octan-3- yl)quinazolin-7-yl)pyrazolo[1,5- a]pyridin-6-ol, TFA | 614 | Column N; condition 30% IPA w/0.1% NH4OH |
| | Peak 2, SFC resolution after STEP 7 | | | |
| Ex. 231b | 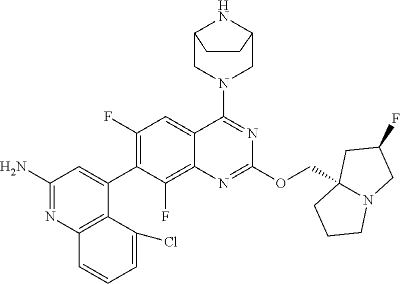 [SMILES: Nc1cc(-c2c(F)cc3c(N4CC5CCC(C4)N5)nc(OC[C@@]45CCCN4C[C@H](F)C5)nc3c2F)c2c(Cl)cccc2n1] | 4-(4-((1R,5S)-3,8-diazabicyclo[3.2.1]oc- tan-3-yl)-6,8-difluoro-2-(((2R,7aS)-2- fluorotetrahydro-1H-pyrrolizin-7a(5H)- yl)methoxy)quinazolin-7-yl)-5- chloroquinolin-2-amine | 610 | Column N; condition: 45% MeOH w/0.1% NH4OH |
| | Peak 2 from SFC | | | |
| Ex. 231a | 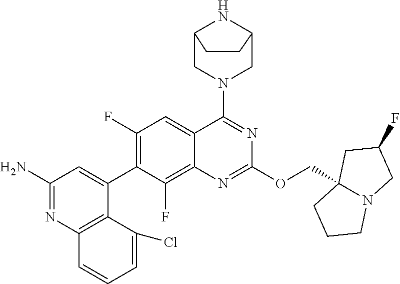 [SMILES: Nc1cc(-c2c(F)cc3c(N4CC5CCC(C4)N5)nc(OC[C@@]45CCCN4C[C@H](F)C5)nc3c2F)c2c(Cl)cccc2n1] | 4-(4-((1R,5S)-3,8-diazabicyclo[3.2.1]oc- tan-3-yl)-6,8-difluoro-2-(((2R,7aS)-2- fluorotetrahydro-1H-pyrrolizin-7a(5H)- yl)methoxy)quinazolin-7-yl)-5- chloroquinazolin-2-amine | 610 | Column N; condition: 45% MeOH w/0.1% NH4OH |
| | Peak 1 from SFC | | | |
| Ex. 232a | 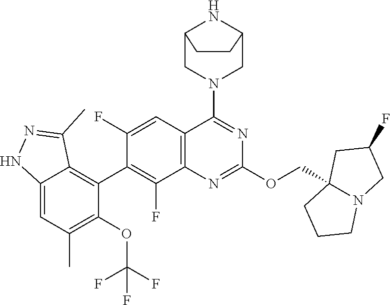 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1OC(F)(F)F] | 4-((1R,5S)-3,8- diazabicyclo[3.2.1]octan-3-yl)-7-(3,6- dimethyl-5-(trifluoromethoxy)-1H- indazol-4-yl)-6,8- difluoro-2-(((2R,7aS)-2- fluorotetrahydro-1H-pyrrolizin-7a(5H)- yl)methoxy)quinazoline | 662 | Column C; Condition 0.1% NH₃H₂O IPA |
| | Peak 1 from SFC after STEP 7 | | | |
| Ex. 232b | 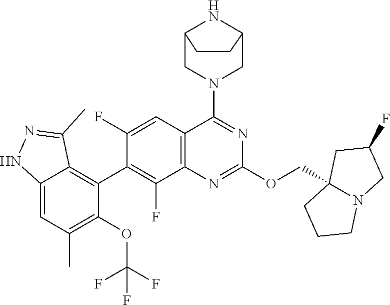 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1OC(F)(F)F] | 4-((1R,5S)-3,8- diazabicyclo[3.2.1]octan-3-yl)-7-(3,6- dimethyl-5-(trifluoromethoxy)-1H- indazol-4-yl)-6,8- difluoro-2-(((2R,7aS)-2- fluorotetrahdyro-1H-pyrrolizin-7a(5H)- yl)methoxy)quinazoline | 662 | Column C; Condition 0.1% NH₃H₂O IPA |
| | Peak 2 from SFC after STEP 7 | | | |
| Ex. 233b | 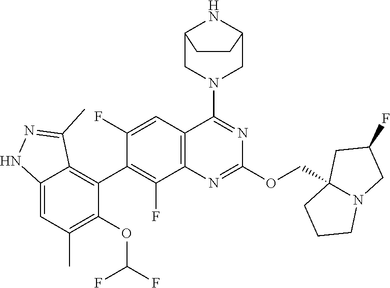 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1OC(F)F] | 4-((1R,5S)-3,8- diazabicyclo[3.2.1]octan-3-yl)-7-(5- (difluoromethoxy)-3,6-dimethyl-1H- indazol-4-yl)-6,8-difluoro-2- (((2R,7aS)-2-fluorotetrahydro-1H- pyrrolizin-7a(5H)- yl)methoxy)quinazoline | 644 | Column(s,s) WHELK-O1 (250 mm*30 mm, 5 um) Condition: 0.1% NH3H2O IPA |
| | Peak 2 | | | |
| Ex. 233a | 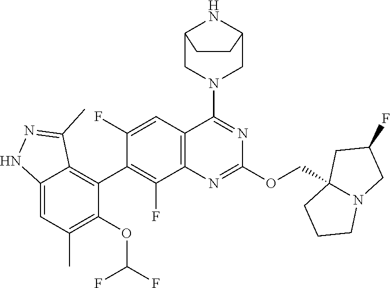 [SMILES: Cc1cc2[nH]nc(C)c2c(-c2c(F)cc3c(N4CC5CCC(C4)N5)nc(OC[C@@]45CCCN4C[C@H](F)C5)nc3c2F)c1OC(F)F] | 4-((1R,5S)-3,8- diazabicyclo[3.2.1]octan-3-yl)-7-(5- (difluoromethoxy)-3,6-dimethyl-1H- indazol-4-yl)-6,8-difluoro-2- (((2R,7aS)-2-fluorotetrahydro-1H- pyrrolizin-7a (5H)-yl)methoxy)quinazoline | 644 | Column(s,s) WHELK-O1 (25 mm*30 mm, 5 um) Condition: 0.1% NH3H2O IPA |
| | Peak 1 | | | |

### Ex 234: Synthesis of 4-((1R,5S)_{3,8}-diazabicyclo[3.2.1]octan-3-yl)-6,8-difluoro-2-(((2R,7aS)-2-fluorotetrahydro-1H-pyrrolizin-7a(5H)-yl)methoxy)-7-(3-methyl-5-(influoromethyl)-1H-pyrazolo[3.4-yl]pyridin-4-yl)quinazoine

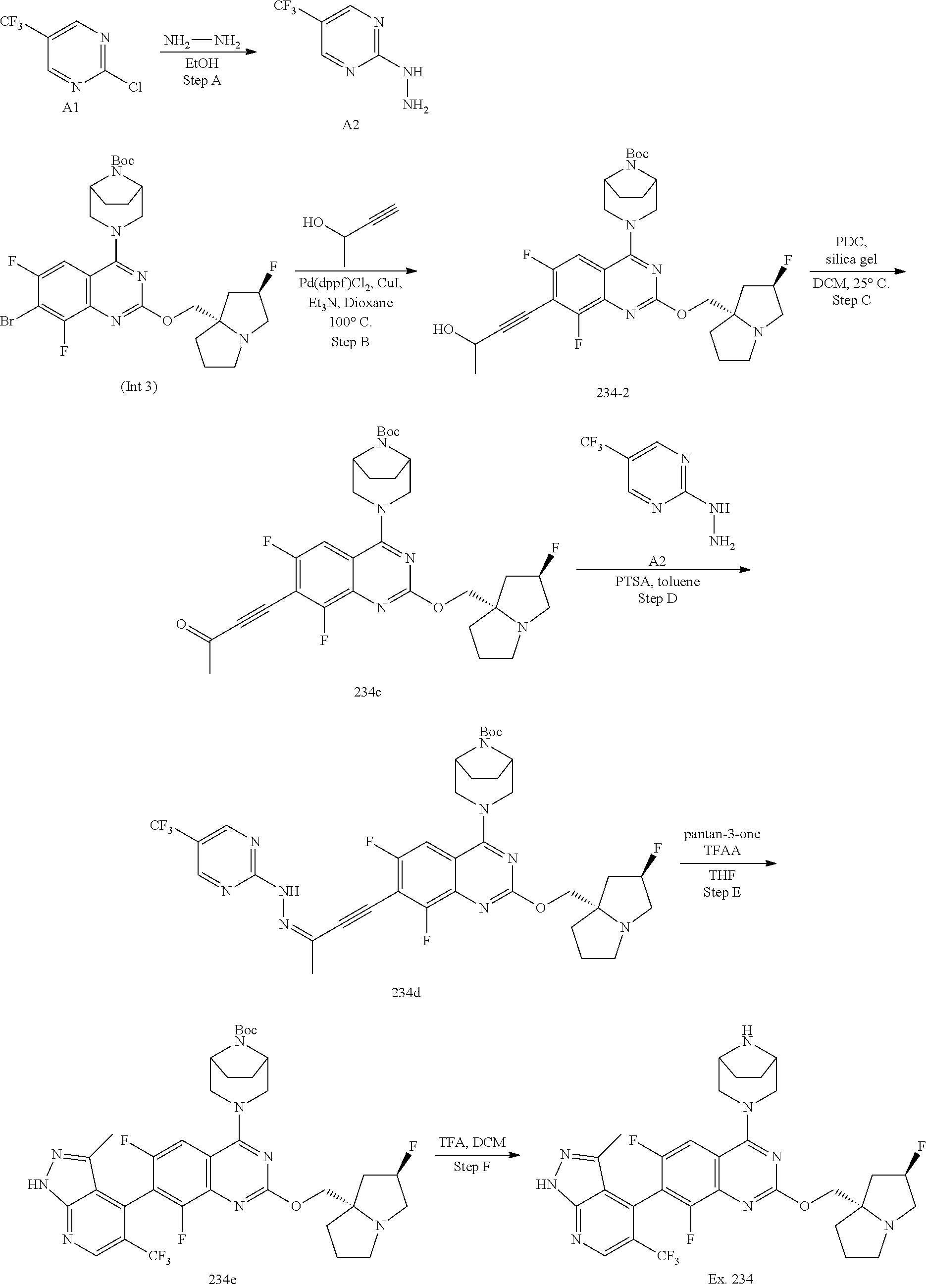

### Step A: 2-hydrazineyl-5-(trifluoromethyl)pyrimidine (A2)

To a solution of 2-chloro-5-(trifluoromethyl)pyrimidine (A1) (200 mg, 1.096 mmol) in ethanol (2 mL) was added hydrazine (0.405 mL, 10.96 mmol)) (85% aqueous solution) at 25° C., and the mixture was stirred at 25° C. for 2 h. The reaction mixture was diluted with EtOAc (20 mL), and washed with brine (5 mL ×3). The combined organic phase was dried over anhydrous Na₂SO₄, filtered, and the filtrate was concentrated in vacuo to give 2-hydrazineyl-5-(trifluoromethyl)pyrimidine (180 mg, 1.011 mmol, 92 yield) as white solid, which was used to next step without further purification. MS (ESI): m/z (M+H)⁺ 179. ¹H NMR (400 MHz, CDCl₃) δ 8.49 (s, 2H), 7.31 (br s, 1H), 4.00 (br s, 2H₁)

### Step B: Tert-butyl (1R,5S)₃-(6,8-difluoro-2-(((2R,7aS)-2-fluorotetrahydro-1H-pyrrolizin-7a(5H)-yl)methoxy)-7-(3-hydroxybut-1-yn-1-yl)quinazolin-4-yl)-3,8-diazabicyclo[3.2.1]octane-8-carboxylate (Int-234-2)

To a solution of tert-butyl (1R,5S)-3-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (500 mg, 0.816 mmol) and but-3-yn-2-ol (229 mg, 3.27 mmol) in dioxane (6 mL) was added copper(I) iodide (15.55 mg, 0.082 mmol), Et₃N (0.228 mL, 1.633 mmol) and PdCl₂(dppf) (59.7 mg, 0.082 mmol), and the mixture was bubbled with N₂ for 1 min. The reaction mixture was stirred at 100° C. for 4 h under N₂. The reaction mixture was concentrated in vacuo, and the residue was purified by flash silica gel chromatography 24 g column, Pet, ether/EtOAc=0/1) to give tert-butyl (1R,5S)-3-(6,8-difluoro-2-(((2R,7aS)-2-fluorotetrahydro-1H-pyrrolizin-7a(5H)-yl)methoxy)-7-(3-hydroxybut-1-yn-1-yl)quinazolin-4-yl)-3,8-diazabicyclo[3.2.1]octane-8-carboxylate. MS (ESI): m/z (M+H)⁺ 602.

### Step C: Tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate (Int-234c)

To a solution of tert-butyl (1R,5S)-3-(6,8-difluoro-2-(((2R,7aS)-2-fluorotetrahydro-1H-pyrrolizin-7a(5H)-yl)methoxy)-7-(3-hydroxybut-1-yn-1-yl)quinazolin-4-yl)-3,8-diazabicyclo[3.2.1]octane-8-carboxylate (Int-234-2) (200 mg, 0.332 mmol) in DCM (8 mL) was added pyridinium dichromate (375 mg, 0.997 mmol) and silica gel (300 mg), and the mixture was stirred at 25° C. for 16 h. The reaction mixture was filtered and the filtrate was concentrated in vacuo, and the residue was purified by preparative TLC plate (SiO₂, Pet.ether/EtOAc=0/1) to give tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate. MS (ESI): m/z (M+H)⁺ 600.

### Step D: Tert-butyl (1R,5S)-3-(6,8-difluoro-2-(((2R,7aS)-2-fluorotetrahydro-1H-pyrrolizin-7a(5H)-yl)methoxy)-7-((Z)-3-(2-(5-(trifluoromethyl)pyrimidin-2-yl)hydrazineyldene)but-1-yn-1-yl)quinazolin-4-yl)-3,8-diazabicyclo[3.2.1]octane-8-carboxylate (Int-234d)

To a solution of tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate (Int-234c) (60 mg, 0.100 mmol) in toluene (1.4 mL) was added 2-hydrazineyl-5-(trifluoromethyl)pyrinmidine (A2) (17.82 mg, 0.100 mmol) and p-toluenesulfonic acid monohydrate (3.81 mg. 0.020 mmol), and the reaction mixture was stirred at 25° C. for 10 h. The reaction mixture was concentrated in vacuo, and the residue was purified by preparative TLC plate (SiO₂, Pet, ether/EtOAc=1/2) to give tert-butyl (1R,5S)-3-(6,8-difluoro-2-(((2R,7aS)-2-fluorotetrahydro-1H-pyrrolizin-7a(5H)-yl)methoxy)-7-((Z)-3-(2-(5-(trifluoromethyl)pyrimidin-2-yl)hydrazineyldene)but-1-yn-1-yl)quinazolin-4-yl)-3,8-diazabicyclo[3.2.1]octane-8-carboxylate. MS (ESI): m/z (M+H)⁺ 760.

### Step E: Tert-butyl (1R,5S)-3-(6,8-difluoro-2-(((2R,7aS)-2-fluorotetrahydro-1H-pyrrolizin-7a(5H)-yl)methoxy)-7-(3-methyl-5-(trifluoromethyl)-1H-pyrazolo[3.4-b]pyridin-4-yl)quinazolin-4-yl)-3,8-diazabicyclo[3.2.1]octane-8-carboxylate (Int-234e)

To a solution of tert-butyl (1R,5S)-3-(6,8-difluoro-2-(((2R,7aS)-2-fluorotetrahydro-1H-pyrrolizin-7a(5H)-yl)methoxy)-7-((Z)-3-(2-(5-(trifluoromethyl)pyrimidin-2-yl)hydrazineyldene)but-1-yn-1-yl)quinazolin-4-yl)-3,8-diazabicyclo[3.2.1]octane-8-carboxylate (Int-234d) (60 mg, 0.079 mmol) and 3-pentanone (102 mg, 1.185 mmol) in THF (5 mL) was added TFAA (0.123 mL, 0.869 mmol), and the mixture was stirred at 80° C. for 10 h. The reaction mixture was concentrated in vacuo, and the residue was purified by preparative HPLC (Column: Boston Green ODS 150*30 mm*5 um: Condition: water (0.1% TFA)-ACN Begin B 45, End B 65 Gradient Time (min) 10, 100% B Hold Time (min) 2 FlowRate (m/min) 25) to give tert-butyl (1R,5S)-3-(6,8-difluoro-2-(((2R,7aS)-2-fluorotetrahydro-1H-pyrrolizin-7a(5H)-yl)methoxy)-7-(3-methyl-5-(trifluoromethyl)-1H-pyrazolo[3.4-b]pyridin-4-yl)quinazolin-4-yl)-3,8-diazabicyclo[3.2.1]octane-8-carboxylate. MS (ESI): m/z (M+H)⁺ 733.

### Step F: 44(1R5S)-3,8-diazabicyclo[3.2.1]octan-3-1)-6,8-difluoro-2-(((2R,7aS)-2-fluorotetrahydro-1H-pyrrolizin-7a(5H)-yl)methoxy)-7-(3-methyl-5-(trifluoromethyl)-1H-pyrazolo[3.4-b]pyridin-4-yl)quinazoline (Ex. 234a) (Peak A)

To a solution of tert-butyl (1R,5S)-3-(6,8-difluoro-2-(((2R,7aS)-2-fluorotetrahydro-1H-pyrrolizin-7a(5H)-yl)methoxy)-7-(3-methyl-5-(trifluoromethyl)-1H-pyrazolo[3.4-b]pyridin-4-yl)quinazolin-4-yl)-3,8-diazabicyclo[3.2.1]octane-8-carboxylate (35 mg, 0.029 mmol) in DCM (1 mL) was added TFA (0.25 mL, 3.24 mmol), and the mixture was stirred at 25° C. for 1 h. LCMS showed the starting material was consumed and desired MS was formed. The reaction mixture was concentrated in vacuo, and the residue was purified by preparative HPLC (Column: Boston Prime C18 150*30 mm*5 um; Condition: water (NH₃H₂O+NH₄HCO₃)-ACN Begin B 56, End B 86 Gradient Time (min) 10, 100% B Hold Time (min) 2 FlowRate (mL/min) 25 to give 4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)-7-(3-methyl-5-(trifluoromethyl)-1H-pyrazolo[3.4-b]pyridin-4-yl)quinazoline. MS (ESI): m/z (M+H)⁺ 633. Ex.234a Peak_A ¹H NMR (400 MHz, MeOD) δ 8.86 (s, 1H), 7.64 (dd, J=1.4, 10.1 Hz, 1H), 5.34-5.08 (m, 1H), 4.43 (br dd, J=6.7, 12.1 Hz, 1H), 4.34-4.25 (m, 1H), 4.23-4.14 (m, 1H), 4.12-4.07 (m, 1H), 3.61-3.42 (m, 4H), 3.19-3.02 (m, 3H), 2.90 (dt, J=5.7, 9.4 Hz, 1H), 2.30-1.98 (m, 3H), 1.94-1.83 (m, 5H), 1.83-1.66 (m, 5H).

### Example 235: 4′-((1R,5S)-3,8-diazabicyclo[3.2.1]octan-3-yl)-5-ethynyl-6′0,8′-difluoro-2′-(((2R,7aS)-2-fluorotetrahydro-1H-pyrrolizin-7a(5H)-yl)methoxy)-[4,7′-biquinazolin]-2-amine

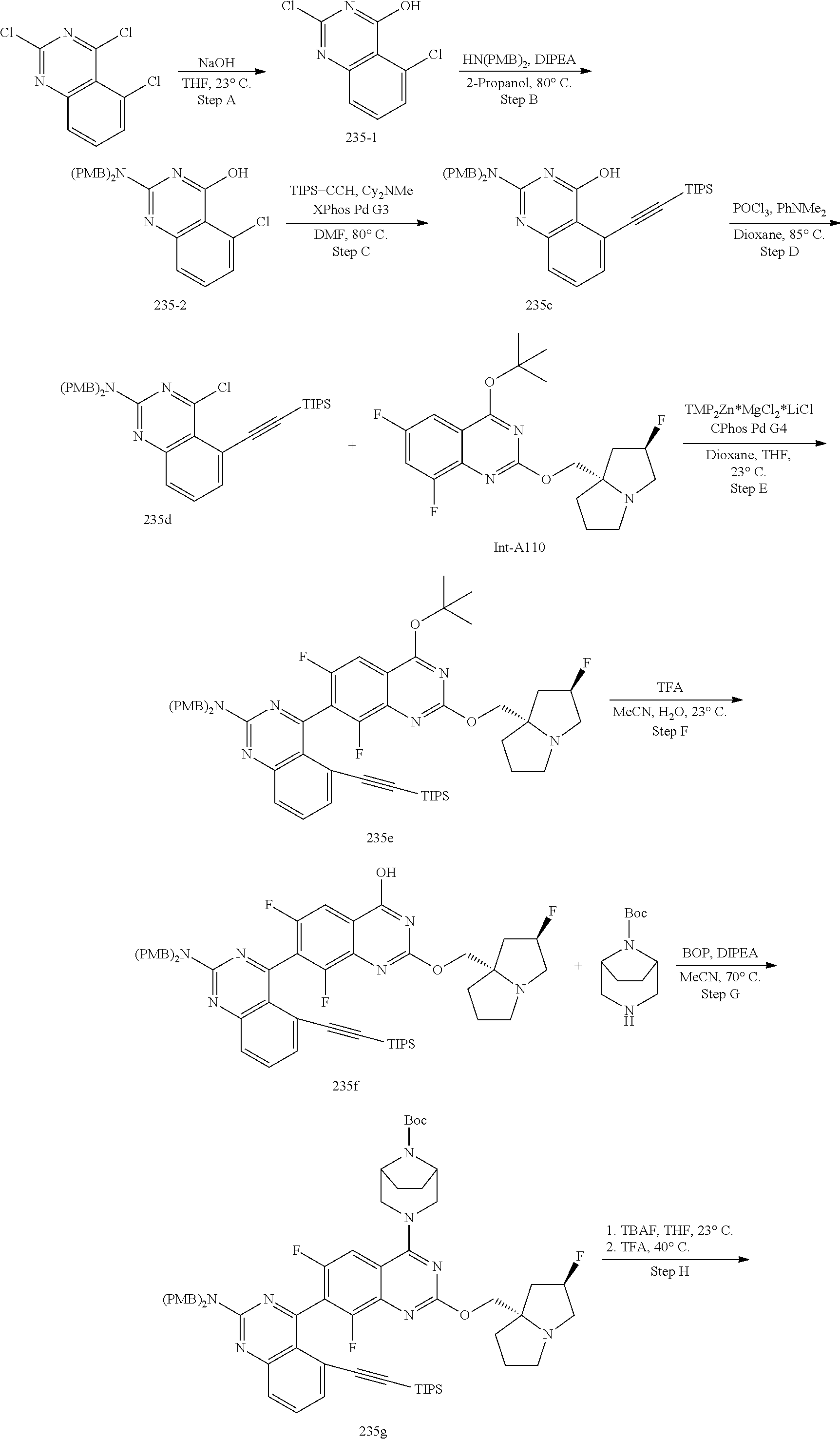

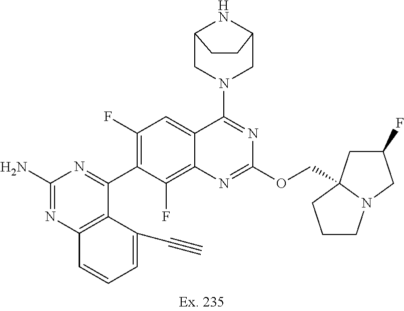 [SMILES: C#Cc1cccc2nc(N)nc(-c3c(F)cc4c(N5CC6CCC(C5)N6)nc(OC[C@@]56CCCN5C[C@H](F)C6)nc4c3F)c12]

(5.0 A:) was then added, acid (2.0 mL, 34 mmol) was added, and the resulting suspension was extracted with ethyl acetate (3 ×30 mL). The combined extracts were washed with sat. aq. NaCi solution (15 mL), dried over anhydrous sodium sulfate, and concentrated under reduced pressure to give 2,5-dichloroquinazolin-4-ol. MS (ESI): m/z (M+H)⁺ 215.

### Step B: 2-(bis(4-methoxybenzyl)amino)-5-(n-3-)-oyezlano-5-chloroquinazolin-4-ol (Int-235-2)

2-Propanol (14 mL), bis(4-methoxybenzyl)amine (1.7 g, 6.6 mmol), and 2,5-dichloroquinazolin-4-ol (Int-235-1) (1.2 g, 5.5 mmol) were combined. The mixture was heated to 80° C. with stirring for 24 h. The mixture was then cooled to 0° C. before it was diluted with ice-cold water (40 mL). The diluted suspension was filtered. The filter cake was rinsed with 20 mL of ice-cold 50% v/v 2-propanol-water. The filter cake was then dried under reduced pressure, giving 2-(bis(4-methoxybenzyl)amino)-5-chloroquinazolin-4-ol. MS (ESI): m/z (M+H)⁺ 436.

### Step C: 2-(bis(4-methoxybenzyl)amino)-5-((triisopropylsilyl)ethynyl)quinazolin-4-ol (Int-235c)

2-(Bis(4-methoxybenzyl)amino)-5-chloroquinazolin-4-ol (Int-235-2) (300 mg, 0.69 mmol), XPhos Pd G3 (290 mg, 0.34 mmol), DMF (3.4 mL), N,N-dicyclohexylmethylamine (670 mg, 3.4 mmol) and ethynyltriisopropylsilane (1,3 g, 6.9 mmol) were combined, and the mixture was deoxygenated with subsurface sparging with dry argon gas for 5 min. The mixture was then heated to 80° C. with stirring under argon for 3.5 h. The mixture was cooled to 23° C. before it was diluted with ethyl acetate (20 mL). The diluted mixture was washed with half-saturated aq. NaCl solution (15 mL). The aqueous wash was extracted with fresh ethyl acetate (2×10 mL). The organic solutions were pooled, washed with sat. aq. NaCl solution (10 mL), dried by passage through a phase-separation column, and concentrated. The residue was purified by flash-column chromatography (24 g silica gel, eluting with hexanes initially, grading to 40% B-hexanes [B=24% v/v ethanol-ethyl acetate]) to provide 2-(bis(4-methoxybenzyl)amino)-5-((triisopropylsilyl)ethynyl)quinazolin-4-ol. MS (ESI): m/z (M+H)⁺ 582.

### Step D: 4-chloro-N,N-bis(4-methoxy benzyl)-5-((triisoprooylsilyl)ethynyl)quinazolin-2-amine (Int-235d)

2-(Bis(4-methoxybenzyl)amino)-5-((triisopropylsilyl)ethyl)quinazolin-4-ol (Int-235c) (150 mg, 0.26 mmol) was dissolved in 1.4-dioxane (650 μL). The solution was treated with N,N-dimethylaniline (78 mg, 0.65 mmol) and phosphoryl trichloride (160 mg, 1.0 mmol). The mixture was heated to 85° C. for 1 h. The mixture was then cooled to 23° C. before it was added to a stirred, ice-cold mixture of sat. aq. sodium bicarbonate solution (30 mL) and ethyl acetate (30 mL). The layers were separated, and the aqueous layer was extracted with fresh ethyl acetate (2×10 mL). The combined extracts were washed with sat. aq. NaCl solution (10 mL), dried by passage through a phase-separation column, and concentrated under reduced pressure. The residue was purified by flash-column chromatography (12 g silica gel, eluting with hexanes initially, grading to 20% ethyl acetate-hexanes) to provide 4-chloro-N,N-bis(4-methoxybenzyl)-5-((triisopropylsilyl)ethynyl)quinazolin-2-amine. MS (ESI): m/z (M+H)⁺ 600.

### Step E: 4′-(tert-butoxy)-6′0,8′-difluoro-2′-(((2R,7aS)-2-fluorotetrahydro-1H-pyrrolizin-7a(5H)-yl)methoxy)-N,N-bis(4-methoxy benzyl)-5-((triisopropylsilyl)ethynl)-[4,7′-biquinazolin]-2-amine (Int-235e)

In a glovebox, a solution of 4-(tert-butoxy)-6,8-difluoro-2-(((2R,7aS)-2-fluorotetrahydro-1H-pyrrolizin-7a(5H)-yl)methoxy)quinazoline (Int-A110) (60 mg, 0.15 mmol) in THF (760 μLL) was added dropwise to a screw-cap vial containing a stirred solution of TMP₂Zn·MgCl₂·LiCl (0.2 M. 1.5 mL, 0.30 mmol) in toluene-THF at 23° C. The vial was sealed, removed from the glovebox, and the contents were stirred for 1 h. In a separate vial, 4-chloro-N,N-bis(4-methoxybenzyl)-5-((triisopropylsilyl)ethynyl)quinazolin-2-amine (Int-235d) (140 mg, 0.23 mmol) and CPhos Pd G4 (19 mg, 0.023 mmol) were combined, and the mixture was dissolved in 1.4-dioxane (760 μL). This solution was added via syringe to the vial containing the freshly prepared aryl-zinc reagent at 23° C. The resulting mixture was stirred for 1.75 h before it was diluted with ethyl acetate (10 mL) and sat. aq. sodium bicarbonate solution (25 mL). The layers were shaken, then separated. The aqueous layer was extracted with fresh ethyl acetate (2×10 mL). The combined organic extracts were dried over anhydrous sodium sulfate, filtered, and concentrated under reduced pressure. The crude residue was purified by flash-column chromatography (12 g silica gel, eluting with hexanes initially, grading to 100% B [B=25% v/v ethanol-ethyl acetate]) to provide 4′-(tert-butoxy)-6′,8′-difluoro-2′-(((2R,7aS)-2-fluorotetrahydro-1H-pyrrolizin-7a(5H)-yl)methoxy)-N,N-bis(4-methoxybenzyl)-5-((triisopropylsilyl)ethynyl)-[4,7′-biquinazolin]-2-amine. MS (ESI): m/z (M+H)⁺ 959.

### Step F: 2-(bis(4-methoxybenzylamino)-6′0,8′-difluoro-2′-(((2R,7aS)-2-fluorotetrahydro-1H-pyrrolizin-7a(5H)-yl)methoxy)-5-((triisopropylsiyl)ethynyl-[4,7′-biquinazolin]-4′-ol (Int-235f)

4′-(Tert-butoxy)-6′,8′-difluoro-2′-(((2R,7aS)-2-fluorotetrahydro-1H-pyrrolizin-7a(5H)-yl)methoxy)-N,N-bis(4-methoxybenzyl)-5-((triisopropylsilyl)ethynyl)-[4,7′-biquinazolin]-2-amine (Int-235e) (120 mg, 0.13 mmol) was dissolved in a mixture of acetonitrile (2.0 mL), water (510 μL), and TFA (51 μL) at 23° C. The solution was stirred for 2 h before it was concentrated under reduced pressure. The residue was diluted with ethyl acetate (10 mL) and sat. aq. sodium bicarbonate solution (10 mL). The layers were shaken, then separated. The aqueous layer was extracted with fresh ethyl acetate (2 ×10 mL). The combined organic extracts were washed with sat. aq. NaCl solution, dried over anhydrous sodium sulfate, and concentrated to provide 2-(bis(4-methoxybenzyl)amino)-6′,8′-difluoro-2′-(((2R,7aS)-2-fluorotetrahydro-1H-pyrrolizin-7a(5H)-yl)methoxy)-5-((triisopropylsilyl)ethynyl)-[4,7′-biquinazolin]-4′-ol. MS (ESI): m/z (M+H)⁺ 903.

### Step G: Tert-butyl (R5S)-3-(2-(bis(4-methoxybenzyl)amino)-6′,8′-difluoro-2′-(((2R,7aS)-2-fluorotetrahydro-1H-pyrrolizin-7a(5H)-yl)methoxy)-5-((triisonroylsilyl ethynyl)-[4,7′-biquinazolin]-4′-yl)-3,8-diazabicyclo[3.2.1]octane-8-carboxylate (Int-235g)

BOP (49 mg, 0, 11 mmol) was added to a stirred solution of 2-(bis(4-methoxybenzyl)amino)-6′,8′-difluoro-2′-(((2R,7aS)-2-fluorotetrahydro-1H-pyrrolizin-7a(5H)-yl)methoxy)-5-((triisopropylsilyl)ethynyl)-[4,7′-biquinazolin]-4′-ol (Int-235f) (50 mg, 0.055 mmol) and DIPEA (48 μL, 0.28 mmol) in acetonitrile (1.1 mL) at 23° C. The solution was stirred for 10 min before tert-butyl (1R,5S)-3,8-diazabicyclo[3.2.1]octane-8-carboxylate (24 mg, 0.11 mmol) was added. The mixture was then heated to 70° C. for 4 h. The mixture was then cooled to 23° C. and the cooled solution was concentrated under reduced pressure. The residue was purified by flash-column chromatography (4 g silica gel, eluting with 20% B/hexanes initially, grading to 80% B/hexanes [B=25% v/v ethanol-ethyl acetate]) to provide tert-butyl (0R,5S)-3-(2-(bis(4-methoxybenzyl)amino)-6′,8′-difluoro-2′-(((2R,7aS)-2-fluorotetrahydro-1H-pyrrolizin-7a(5H)-y)methoxy)-5-((triisopropylsilyl)ethynyl)-[4,7′-biquinazolin]-4′-yl)-3,8-diazabicyclo[3.2.1]octane-8-carboxylate. MS (ESI): m/z (M+H)⁺ 1097.

### Step H: 4′-((1R,5S)-3,8-diazabicyclo[3.2.1]octan-3-yl)-5-ethynyl-6′0,8′-difluoro-2′-(((2R,7aS)-2-fluorotetrahydro-1H-pyrrolizin-7a(5H)-yl)methoxy)-[4,7′-biquinazolin]-2-amine (Ex.235)

Tetrabutylammonium fluoride (1 M solution in THF, 130 LL, 0.13 mmol) was added to a stirred solution of tert-butyl (1R,5S)-3-(2-(bis(4-methoxybenzyl)amino)-6′,8′-difluoro-2′-(((2R,7aS)-2-fluorotetrahydro-1H-pyrrolizin-7a(5H)-yl)methoxy)-5-((triisopropylsilyl)ethynyl)-[4,7′-biquinazolin]-4′-yl)-3,8-diazabicyclo[3.2.1]octane-8-carboxylate (Int-235g) (35 mg, 0.032 mmol) in THF (640 μL) at 23° C. The mixture was stirred for 17 h. The mixture was then diluted with 5 mL sat. aq. NaCl solution and 5 mL sat. aq. sodium bicarbonate solution. The resulting mixture was extracted with ethyl acetate (3×10 mL). The combined extracts were washed with sat. aq. NaCl solution (10 mL), dried over anhydrous sodium sulfate, filtered, and concentrated under reduced pressure. The crude residue was dissolved in trifluoroacetic acid (4.0 mL). The resulting solution was heated to 40° C. for 24 h before it was concentrated under reduced pressure. The residue was purified by mass-triggered reverse-phase HPLC (acetonitrile-water with TFA as modifier) to provide 4′-((1R,5S)-3,8-diazabicyclo[3.2.1]octan-3-yl)-5-ethynyl-6′,8′-difluoro-2′-(((2R,7aS)-2-fluorotetrahydro-1H-pyrrolizin-7a(5H)-yl)methoxy)-[4,7′-biquinazolin]-2-amine as the TFA salt. ¹H NMR (500 MHz, CD₃OD) δ 7.84 (t, J=7.9 Hz, 1H), 7.73 (d, J=8.5 Hz, 1H), 7.69 (d, J=9.8 Hz, 1H), 7.57 (d, J=7.3 Hz, 1H). 5.58 (d,.=51.8 Hz, 1H), 4.72-4.58 (m, 4H), 4.25 (d, J=10.5 Hz, 2H), 4.03-3.78 (m, 5H), 3.47 (td, J=10.6, 5.7 Hz, 1H), 3.36 (d, J=4.3 Hz, 1H), 2.75-2.56 (m, 2H), 2.47-2.41 (m, 1H), 2.38-2.32 (m, 2H), 2.22-2.15 (m, 5H). MS (ESI): m/z (M+H)⁺ 601.

### Ex 236: Synthesis of 6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-vi)methoxyquinazolin-7-yl)-3-fluoro-4-methyl-5-(trinfluoromethyl)pyridin-2-amine

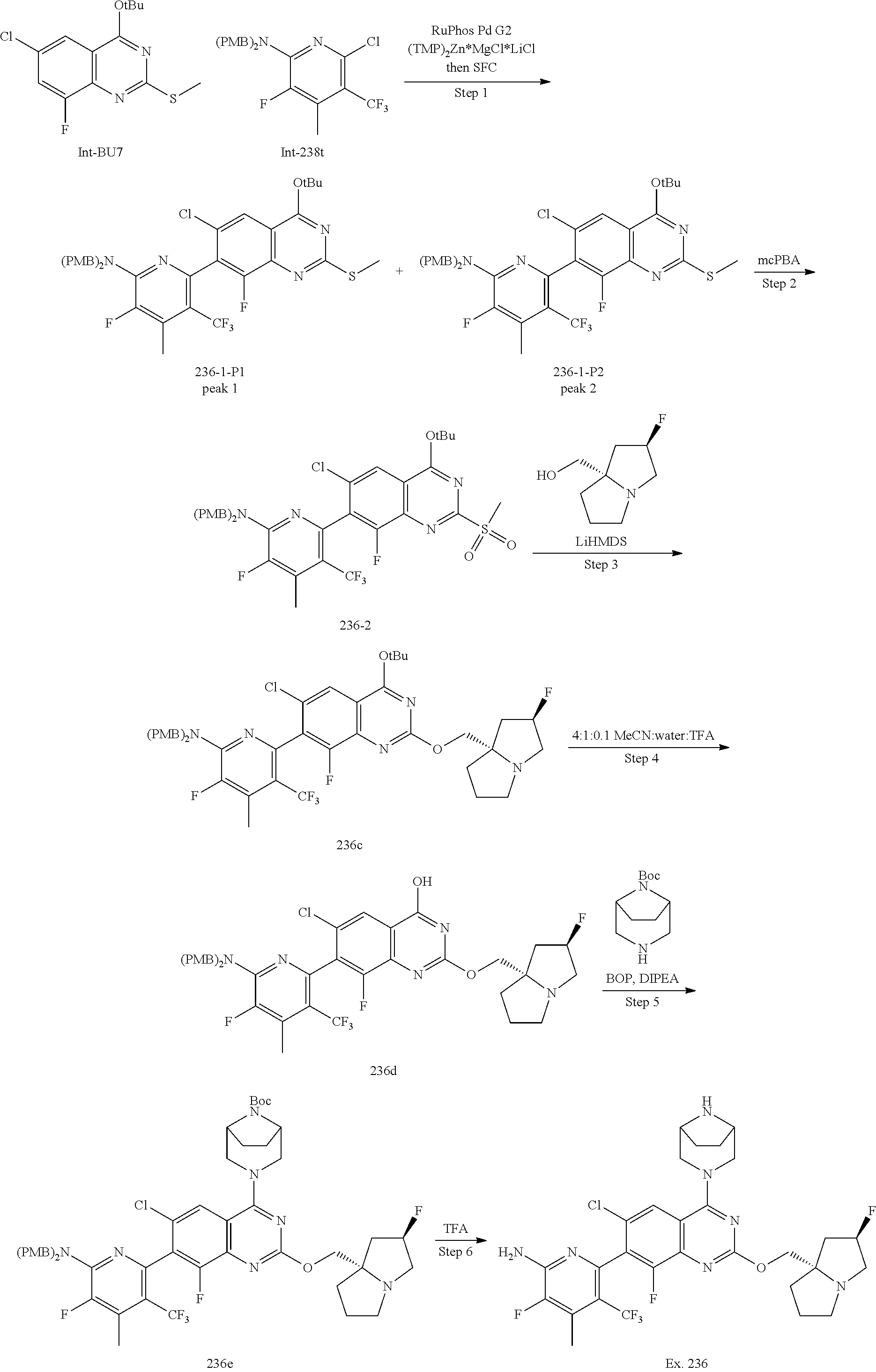

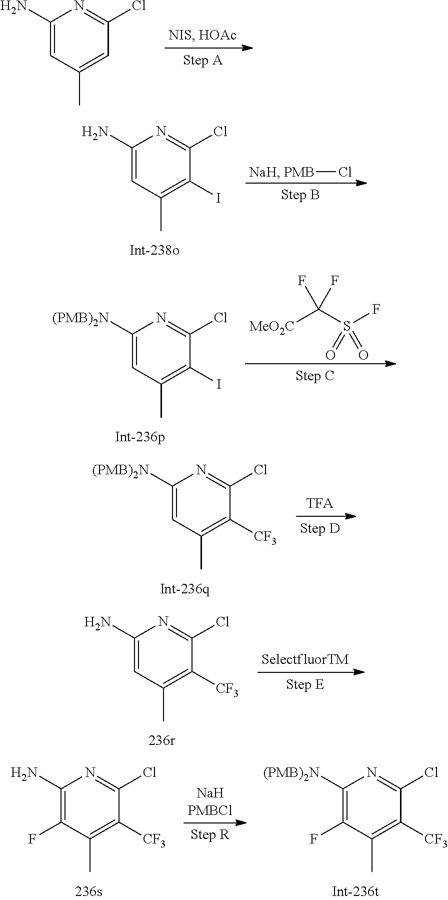

### Intermediate (236t): 6-chloro-3-fluoro-N,N-bis(4-methosybenlzl)-4-methyl-5-(trifluoromethyl) pyridin-2-amine (3 steps- Int-236t)

### Step A (2360): 6-chloro-5-iodo-4-methylpyridin-2-amine (Int-236o)

To a solution of 6-chloro-4-methylpyridin-2-amine (13 g, 91 mmol) in DMF (182 mL) was added N₁S (20.51 g, 91 mmol). The resultant reaction mixture was allowed to stir at 80° C. The reaction mixture was partitioned between EtOAc/water. The phases were separated and the aqueous phase extracted with EtOAc. The combined organic layers were then washed with brine, dried over Na₂SO₄, and concentrated under reduced pressure. The residue was purified by flash chromatography over silica (0-10% EtOAc: Pet, ether) to afford 6-chloro-5-iodo-4-methylpyridin-2-amine. [M+H]⁺ Found: 269.

### Step B (236 P): 6-chloro-5-iodo-N,N-bis(4-methoxybenzyl)-4-methylnpyridin-2-amine (Int 236p)

A mixture of 1-(chloromethyl)-4-methoxybenzene (16.33 g, 104 mmol), 6-chloro-5-iodo-4-methylpyridin-2-amine (Int 236o) (7g, 26.1 mmol) and NaH (8.34 g, 209 mmol) in DMF (30 ml) was allowed to stir at 25° C. for 6 h. The reaction mixture was partitioned between EtOAc/water. The phases were separated and the aqueous phase extracted with EtOAc. The combined organic layers were then washed with brine, dried over Na₂SO₄, and concentrated under reduced pressure. The residue was purified by flash chromatography over silica (0-20% EtOAc: pet, ether) to afford 6-chloro-5-iodo-N,N-bis(4-methoxybenzyl)-4-methylpyridin-2-amine. [M+H]⁺ Found: 509.

### Step C (236 Q): 6-chloro-N,N-bis(4-methoxybenzyl)-4-methyl-5-(trifluoromethyl)pyridin-2-amine (Int 236a)

A mixture of 6-chloro-5-iodo-N,N-bis(4-methoxybenzyl)-4-methylpyridin-2-amine (Int-236p) (500 mg, 0.983 mmol), methyl 2,2-difluoro-2-(fluorosulfonyl)acetate (378 mg, 1.965 mmol) and copper(I) iodide (374 mg, 1.965 mmol) in DMF (10 ml) was allowed to stir at 90° C. for 2 h. The reaction mixture was partitioned between EtOAc/water. The phases were separated and the aqueous phase extracted with EtOAc. The combined organic layers were then washed with brine, dried over Na₂SO₄, and concentrated under reduced pressure. The residue was purified by flash chromatography over silica (0-20% EtOAc: Pet, ether) to afford 6-chloro-N,N-bis(4-methoxybenzyl)-4-methyl-5-(trifluoromethyl)pyridin-2-amine. [M+H]⁺ Found: 451.

### Step D: 6-chloro-4-methyl-5-(trifluoromethyl)pyridin-2-amine (Int-236r)

6-Chloro-N,N-bis(4-methoxybenzyl)-4-methyl-5-(trifluoromethyl)pyridin-2-amine (Int-236q) (1.05 g, 33.3 mmol) in TFA (33.3 mL) was stirred at 50° C. for 2 hours. The reaction was concentrated under reduced pressure and the residue was diluted with DCM, cooled to 0° C., and diluted with a saturated solution of NaHCO₃. The mixture was extracted with DCM, and the combined organic layers were dried over Na₂SO₄, filtered, and concentrated under reduced pressure. The residue was purified by silica gel column chromatography (0-5% DCM in EtOAc) to afford 6-chloro-4-methyl-5-(trifluoromethyl)pyridin-2-amine. [M+H]⁺ Found: 211.

### Step E: 6-chloro-3-fluoro-4-methyl-5-(trifluoromethyl)pyridin-2-amine (Int-236s)

6-Chloro-4-methyl-5-(trifluoromethyl)pyridin-2-amine (Int-246r) (1.60 g, 7.60 mmol) and Selectfluor™ (3.23 g, 9.12 mmol) were suspended in MeCN (50.0 mL) and stirred at 23° C. for 18 hours. The reaction was diluted with a saturated solution of NaHCO₃, extracted with EtOAc, and the combined organic layers were washed with brine, dried over Na₂SO₄, filtered, and concentrated under reduced pressure. The residue was purified by silica gel column chromatography (0-20% EtOAc in hexanes) to afford 6-chloro-3-fluoro-4-methyl-5-(trifluoromethyl)pyridin-2-amine. [M+H]⁺ Found: 229.

### Step F: 6-chloro-3-fluoro-N,N-bis(4-methoxybenzyl)-4-methyl-5-(trifluoromethyl)pyridin-2-amine (Int-236t)

60% NaH in mineral oil (454 mg, 11.4 mmol) was added to a solution of 6-chloro-3-fluoro-4-methyl-5-(trifluoromethyl)pyridin-2-amine (Int-246s) (865 mg, 3.78 mmol) in DMF (18.9 mL) cooled to 0° C. The mixture was stirred at 0° C. for 1 hour. 4-Methoxybenzyl chloride (1.08 ml. 7.95 mmol) was added, and the mixture was stirred at 23° C. for 1 hour. The mixture was diluted with a saturated solution of NH₄Cl, extracted with EtOAc, and the combined organic layers were washed with water, brine, dried over Na₂SO₄, filtered, and concentrated under reduced pressure. The residue was purified by silica gel column chromatography (0-5% EtOAc in hexanes) to afford 6-chloro-3-fluoro-N,N-bis(4-methoxybenzyl)-4-methyl-5-(trifluoromethyl)pyridin-2-amine. [M+H]⁺ Found: 469.

### Step 1: 6-(4-(tert-butoxy)-6-chloro-8-fluoro-2-(methylthio)quinazolin-7-yl)-3-fluoro-N,N-bis(4-methoxybenzyl)-4-methyl-5-(trifluoromethyl)pyridin-2-amine (Int-236-1-P1 and Int-236-1-P2)

4-(tert-butoxy)-6-chloro-8-fluoro-2-(methylthio)quinazoline (Int-BU7) (350 mg, 1.16 mmol) in THF (5.82 mL) was added to a flask containing bis(2,2,6,6-tetramethylpiperidinyl)zinc, lithium chloride, magnesium chloride (11.7 mL, 2.33 mmol) and the mixture was stirred at 50° C. for 3 hours. In a separate flask, RuPhos Pd G2 (136 mg, 0.175 mmol) was added to 6-chloro-3-fluoro-N,N-bis(4-methoxybenzyl)-4-methyl-5-(trifluoromethyl)pyridin-2-amine (Int-236t) (818 mg, 1.75 mmol) in 1.4-dioxane (5.82 mL) and was sonicated until the solution became homogenous. The Pd mixture was added dropwise to the flask containing the zincate species and the resulting mixture was stirred at 80° C. for 2 hours. The mixture was diluted with a saturated solution of NaHCO₃, extracted with EtOAc, and the combined organic layers were washed with brine, and dried over Na₂SO₄, filtered, and concentrated under reduced pressure. The residue was purified by silica gel column chromatography (0-50% EtOAc in hexanes). The residue was purified by SFC (Column: AS-H, 21×250 mm, Eluent:1:1 MeOH: MeCN) to afford 6-(4-(tert-butoxy)-6-chloro-8-fluoro-2-(methylthio)quinazolin-7-yl)-3-fluoro-N,N-bis(4-methoxybenzyl)-4-methyl-5-(trifluoromethyl)pyridin-2-amine (peak 1; 2.20 min, Int-246-1-P1) and 6-(4-(tert-butoxy)-6-chloro-8-fluoro-2-(methylthio)quinazolin-7-yl)-3-fluoro-N,N-bis(4-methoxybenzyl)-4-methyl-5-(trifluoromethyl)pyridin-2-amine (peak 2: 3.55 min, Int-236-1-P2). [M+H]⁺ Found: 734.

The following synthetic steps were done with peak 1:

### Step 2: 6-(4-(tert-butoxy)-6-chloro-8-fluoro-2-(methylsulfonyl)quinazolin-7-yl)-3-fluoro-N,N-bis(4-methoxybenzyl)-4-methyl-5-(trifluoromethyl)pyridin-2-amine (Int-236-2)

6-(4-(tert-butoxy)-6-chloro-8-fluoro-2-(methylthio)quinazolin-7-yl)-3-fluoro-N,N-bis(4-methoxybenzyl)-4-methyl-5-(trifluoromethyl)pyridin-2-amine (Int-246-1-P1) (143 mg, 0.195 mmol) in DCM (1.95 mL) was cooled toO ° C. mCPBA (90.0 mg, 0.390 mmol) was added and the mixture was stirred at 25° C. for 2 hours. The mixture was diluted with a saturated solution of NaHCO₃, extracted with DCM, and the combined organic layers were dried over Na₂SO₄, filtered and concentrated under reduced pressure to afford 6-(4-(tert-butoxy)-6-chloro-8-fluoro-2-(methylsulfonyl)quinazolin-7-yl)-3-fluoro-N,N-bis(4-methoxybenzyl)-4-methyl-5-(trifluoromethyl)pyridin-2-amine. The material was used without further purification. [M+H]₁ Found: 765.

### Step 3: 6-(4-(tert-butoxy)-6-chloro-8-fluoro-2-(((2R,7aS)-2-fluorotetrahydro-1H-pryrrolizin-7a(5H)-yl)methoxy)quinazolin-7-yl)-3-fluoro-N,N-bis(4-methoxybenzyl)-4-methyl-5-(trifluoromethyl)pyridin-2-amine (Int-236c)

((2R,7aS)-2-Fluorotetrahydro-1H-pyrrolizin-7a(5H)-yl)methanol (Int-A18) (41.9 mg, 0.263 mmol) in THF (1.10 mL) was cooled to −30° C. 1.0 M LiHMDS in THF (190 μl, 0.285 mmol) was added dropwise and the mixture was stirred at −30° C. for 30 minutes. The resulting mixture was added to 6-(4-(tert-butoxy)-6-chloro-8-fluoro-2-(methylsulfonyl)quinazolin-7-yl)-3-fluoro-N,N-bis(4-methoxybenzyl)-4-methyl-5-(trifluoromethyl)pyridin-2-amine (Int-236-2) (168 mg, 0.220 mmol) in THF (1.10 mL) cooled to −30° C. The mixture was warmed to 25° C. and stirred for 1 hour. Another solution of ((2R,7aS)-2-fluorotetrahydro-1H-pyrrolizin-7a(5H)-yl)methanol (Int-A1S) (41.9 mg, 0.263 mmol) in THF (1.10 mL) and 1.0 M LHMDS in THF (190 μl, 0.285 mmol) was made and added to the mixture. The reaction was stirred at 25° C. for 30 minutes and then diluted with a saturated solution of NaHCO; The mixture was extracted with EtOAc, and the combined organic layers were washed with brine, dried over Na₂SO₄, filtered, and concentrated under reduced pressure. The residue was purified by silica gel column chromatography (0-100% EtOAc in hexanes) to afford 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-3-fluoro-N,N-bis(4-methoxybenzyl)-4-methyl-5-(trifluoromethyl)pyridin-2-amine. [M+H]⁺Found: 844.

### Step 4: 7-(6-(bis(4-methoxybenzyl)amino)-5-fluoro-4-methyl-3-(trifluoromethyl)l)pyridin-2-yl)-6-chloro-8-fluoro-2-(((2R,7aS)-2-fluorotetrahydro-1H-pyrrolizin-7a(5H)-yl)methoxy)quinazolin-4-ol (Int-236d)

6-(4-(Tert-butoxy)-6-chloro-8-fluoro-2-(((2R,7aS)-2-fluorotetrahydro-1H-pyrrolizin-7a(5H)-yl)methoxy)quinazolin-7-yl)-3-fluoro-N,N-bis(4-methoxybenzyl)-4-methyl-5-(trifluoromethyl)pyridin-2-amine (Int-246c) (86.3 mg, 0.102 mmol) was dissolved in MeCN (648 μL), water (162 μL) and TFA (8.10 μL), and the mixture was stirred at 25° C. for 4 hours. The mixture was diluted with a saturated solution of NaHCO₃, extracted with EtOAc, and the combined organic layers were washed with brine, dried over Na₂SO₄, filtered, and concentrated under reduced pressure to afford 7-(6-(bis(4-methoxy benzyl)amino)-5-fluoro-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol. The material was used without further purification. [M+H]⁺ Found: 788.

### Step 5: Tert-butyl (1R,5S)-3-(7-(6-(bis(4-methoxybenzyl)amino)-5-fluoro-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-236e)

To a flask containing 7-(6-(bis(4-methoxybenzyl)amino)-5-fluoro-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-236d) (64.8 mg, 0.082 mmol) and MeCN (1.64 mL) was added BOP (54.5 mg, 0.123 mmol), followed by DIPEA (71.8 μl, 0.411 mmol) and tert-butyl (1R,5S)-3,8-diazabicyclo[3.2.1]octane-8-carboxylate (17.5 mg. 0.082 mmol). The mixture was heated to 80° C. for 1 hour. The mixture was diluted with a saturated solution of NaHCO₃, extracted with DCM, and the combined organic layers were dried over Na₂SO₄, filtered, and concentrated under reduced pressure to afford tert-butyl (1R,5S)-3-(7-(6-(bis(4-methoxybenzyl)amino)-5-fluoro-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. The material was used without purification. [M+H] Found: 982. Step 6: 6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-ylmethoxy)quinazolin-7-yl)-4-methyl-5-(trifluoromethyl)pyridin-2-amine (Ex.236)

Tert-butyl (1R,5S)-3-(7-(6-(Bis(4-methoxybenzyl)amino)-5-fluoro-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-236e) (80.4 mg, 0.082 mmol) was dissolved in TFA (633 μL, 8.22 mmol) and the reaction mixture was stirred at 50° C. for 2 hour. The mixture was diluted with a saturated solution of NaHCO₃, extracted with DCM, and the combined organic layers were dried over Na₂SO₄, filtered, and concentrated under reduced pressure. The residue was dissolved in DMSO (2.00 mL), filtered, and purified by mass triggered reverse phase HPLC (MeCN/water with 0.1% TFA modifier, linear gradient). The residue was dissolved in DMSO (2.00 mL), filtered, and purified by mass triggered reverse phase HPLC (MeCN/water with 0.1% NH₄OH modifier, linear gradient) to afford 6-(4-((1R,5.S-3,8-diazabicyclo[3.2.]octan-3-yl)-6-chloro-8-fluoro-2-(((2R,7aS)-2-fluorotetrahydro-1H-pyrrolizin-7a(5H)-yl)methoxy)quinazolin-7-yl)-3-fluoro-4-methyl-5-(trifluoromethyl)pyridin-2-amine (Ex.236a). [M+H]⁺ Found: 642. ¹H NMR (600 MHz, DMSO-d₆) δ 7.81 (s, 1H), 7.21 (s, 2H), 5.26 (d, J=54.0 Hz, 1H), 4.23 (dd, J=81.1, 11.7 Hz, 2H), 4.09-3.95 (m, 2H), 3.51 (dd, J=20.8, 11.6 Hz, 4H), 3.12-2.99 (m, 3H), 2.81 (d, J=6.2 Hz, 1H), 2.34 (s, 3H), 2.12-1.95 (m, 3H), 1.91-1.71 (m, 3H), 1.59 (d, J=32.6 Hz, 4H).

The above steps was repeated with peak 2 from step 1 to afford 6-(4-((1R,5S)-3,8-diazabicyclo[3.2.1]octan-3-yl)-6-chloro-8-fluoro-2-(((2R,7a9-2-fluorotetrahydro-1H-pyrrolizin-7a(5H)-yl)methoxy)quinazolin-7-yl)-3-fluoro-4-methyl-5-(trifluoromethyl)pyridin-2-amine (Ex.236b). [M+H]⁺ Found: 642. ¹H NMR (600 MHz, DMSO-dr) 5 7.81 (s, 1H), 7.21 (s, 2H), 5.26 (d, J=54.2 Hz, 1H), 4.30 (d, J=11.9 Hz, 1H), 4.17 (d, J=11.8 Hz, 1H), 4.07 (d, J=10.3 Hz, 1H), 3.96 (d, J=10.3 Hz, 1H), 3.55-3.42 (m, 5H), 3.12-2.99 (m, 3H), 2.86-2.78 (m, 1H), 2.34 (s, 2H), 2.17-1.97 (m, 3H), 1.90-1.70 (m, 3H), 1.59 (d, J=30.4 Hz, 3H).

Examples in the table below were synthesized following a similar procedure as Ex. 236.

| | | | | [M + | SFC |
|---|---|---|---|---|---|
| | | | | H]⁺ | Purification |
| Ex. | Starting Material | Structure | Compound Name | Found | Conditions |
| Ex. 237 | 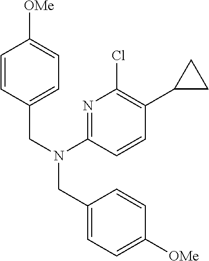 [SMILES: COc1ccc(CN(Cc2ccc(OC)cc2)c2ccc(C3CC3)c(Cl)n2)cc1] | 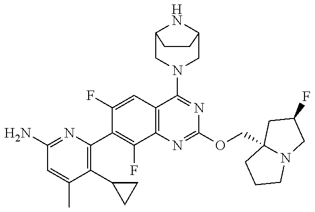 [SMILES: Cc1cc(N)nc(-c2c(F)cc3c(N4CC5CCC(C4)N5)nc(OC[C@@]45CCCN4C[C@H](F)C5)nc3c2F)c1C1CC1] | 6-(4-((1R,5S)-3,8-di- azabicyclo[3.2.1]octan-3- yl)-6,8-difluoro-2- (((2R,7aS)-2- fluorotetrahydro- 1H-pyrrolizin-7a(5H)- yl)methoxy)quinazolin-7- yl)-5-cyclopropyl-4- methylpyridin-2-amine | 580 | NA |
| Ex. 238b | Int 236q | 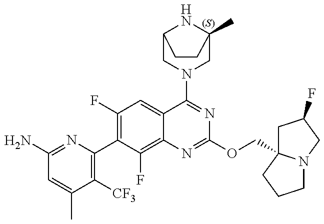 | 2,2,2-trifluoroacetalde- hyde-6-(6-chloro- 8-fluoro-2-(((2R,7aS)- 2-fluorotetrahydro-1H- pyrrolizin-7a(5H)- yl)methoxy)- 4-((1S,5R)-1-methyl-3,8- diazabicyclo[3.2.1]octan-3- yl)quinazolin-7-yl)- 4-methyl-5-(trifluoro- methyl)pyridin-2- amine (1/1) | 639 | Column A; Condition: 0.1% NH3H2O ETOH |
| | | Peak 2 | | | |
| Ex. 238a | Int 236q | 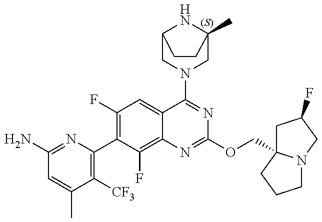 | 6-(6-chloro-8-fluoro- 2-(((2R,7aS)-2-fluoro- tetrahydro-1H-pyrrolizin- 7a(5H)-yl)methoxy)-4- ((1S,5R)-1-methyl-3,8- diazabicyclo[3.2.1]octan- 3-yl)quinazolin-7-yl)- 4-methyl-5-(trifluoro- methyl)pyridin-2-amine | 639 | Column A; Condition: 0.1% NH3H2O ETOH |
| | | Peak 1 | | | |

### Example 102: 4-(6,8-difluoro-2-(((2R-7aS)-2-fluorotetrahydro-1H-pyrrolizin-7a(5H)-yl)methoxy)-4-((1S,5R)-1-(S)-methyl-3,8-diazabicyclo[3.2.1]octan-3-yl)quinazolin-7-yl)-5-ethynylnaphthalen-2-ol (Ex. 102)

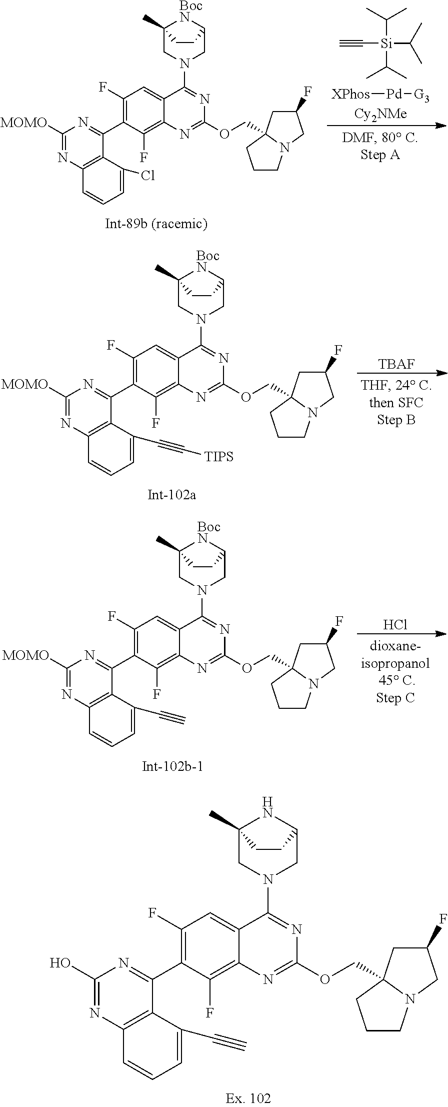

### Step A: Tert-butyl (1S,5R)-3-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl))quinazolin-4-yl)-1-methyl-3,8-diazabicyclo[3.2.1]octane-8-carboxylate (Int-102a)

Tert-butyl (1S,5R)-3-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-89b, racemic) (110 mg, 0.143 mmol), ethynyltriisopropylsilane (321 μL, 1.43 mmol). XPhos Pd G3 (61 mg, 0.072 mmol), N-cyclohexyl-N-methyl)cyclohexanamine (140 mg, 0.716 mmol) were added into a 20 mL vial. The reaction vessel was evacuated and backfilled with a balloon of nitrogen three times. DMF (1.4 mL) was added into the reaction vessel. The resulting mixture was heated at 80° C. for 16 h. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with a 12-g amino-functionalized silica gel column, eluting with hexanes initially, grading to 50% ethyl acetate-hexanes, linear gradient to afford the desired product tert-butyl (1S,5R)-3-(6,8-difluoro-2-(((2R,7aS)-2-fluorotetrahydro-1H-pyrrolizin-7a(5H)-yl)methoxy)-7-(3-(methylmethoxy)-8-((triisopropylsilyl)ethynyl)naphthalen-1-yl)quinazolin-4-yl)-1-methyl-3,8-diazabicyclo[3.2.1]octane-8-carboxylate (Int-102a). MS (ESI): m/z (M+H)⁺ 914.

### Step B: Tert-butyl (1S,5R)-3-(7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-102b-1)

Tert-butyl (1S,5R)-3-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-1-methyl-3,8-diazabicyclo[3.2.1]octane-8-carboxylate (Int-102a, 110 mg, 0.0887 mmol) was added in a 20 mL vial. The reaction vessel was evacuated and backfilled with a balloon of nitrogen three times. THF (887 μL) was added into the reaction vessel. A 1 M THF solution of tetrabutylammonium fluoride (89 μL, 0.089 mmol) was added into the reaction vessel. The resulting mixture was stirred for 5 min. The product mixture was diluted with ethyl acetate (100 mL). The diluted product mixture was washed three times with saturated sodium bicarbonate aqueous solution (3×20 mL). The organic layer was dried over sodium sulfate. The dried solution was filtered and the filtrate was concentrated to dryness. The residue obtained was purified by flash column chromatography with 40-g silica gel column, eluting with hexanes initially, grading to 100% ethyl acetate: ethanol =3:1, linear gradient. The racemic material was resolved by SFC (Column Q; Conditions: 30% isopropanol with 0.1% NH₄OH) to afford peak 1 as tert-butyl (1S, 5R)-3-(7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-102b-1). And peak 2 as tert-butyl (1S,5R)-3-(7-(8-ethynyl-3-(methylmethoxy)naphthalen-1-yl)-6,8-difluoro-2-(((2R,7aS)-2-fluorotetrahydro-1H-pyrrolizin-7a(5H)-yl)methoxy)quinazolin-4-y)-1-methyl-3,8-diazabicyclo[3.2.1]octane-8-carboxylate (Int-102b-2). MS (EST): m/z (M+H)⁺ 758.

### Step C: 4-(6,8-difluoro-2-(((2R,7aS)-2-fluorotetrahydro-1H-pyrrolizin-7a(5H)-yl)methoxy)-4-((1S,5R)-1-(S)-methyl-3,8-diazabicyclo[3.2.1]octan-3-yl)quinazolin-7-yl)-5-ethynylnaphthalen-2-ol (Ex. 102)

Tert-butyl -(1S,5R)-3-(7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-methyl-3,8-diazabicyclo[3.2.1]octane-8-carboxylate (Int-102b-1, 9.0 mg, 0.012 mmol) was added in an 8 mL vial. The reaction vessel was evacuated and backfilled with a balloon of nitrogen three times. 2-Propanol (264 μL) was added into the reaction vessel to dissolve the starting material. Then 4 N HCl solution in dioxane (132 μL) was added dropwise. The resulting mixture was heated at 45° C. for 1 h. The product mixture was concentrated to dryness. The residue was purified using preparatory reverse phase prep-HPLC (MeCN/water with 0.1% TFA modifier). The dried down fraction was re-lyophilized to afford 4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-5-ethynylnaphthalen-2-ol (Ex. 102). MS (ESI): m/z (M+H)⁺ 614. ¹H NMR (500 MHz, DMSO-d) δ 10.84 (s, 1H), 10.27 (s, 1H), 9.53-9.10 (m, 2H), 7.93 (dd, J=8.3, 1.5 Hz, 1H), 7.71 (d, J=9.8 Hz, 1H), 7.57-7.36 (m, 3H), 7.08 (d, J=2.5 Hz, 1H), 5.57 (d, J=52.3 Hz, 1H), 4.59 (s, 2H), 4.44 (d, J=13.7 Hz, 1H), 4.32 (d, J=13.7 Hz, 1H), 4.23 (s, 1H), 3.80 (dd, J=60.0, 15.5 Hz, 4H), 3.59 (d, J=16.3 Hz, 2H), 2.69-2.53 (m, 2H), 2.42-2.27 (m, 1H), 2.26-1.94 (m, 6H), 1.87-1.69 (m, 1H), 1.53 (s, 3H).

### STEP D: 4-(68-difluoro-2-(((2R,7aS)-2-fluorotetrahydro-1H-pyrrolizin-7a(5H)-yl)methoxy)-4-((1S,5R)-1-(S)-methyl-3,8-diazabicyclo[3.2.1]octan-3-yl)quinazolin-7-yl)-5-ethynylnaphthalen-2-ol. 2TFA (Ex. 102)

Tert-butyl (1S,5R)-3-(7-(8-ethynyl-3-(methoxymethyl)naphthalen-1-yl)-6,8-difluoro-2-(((2R,7aS)-2-fluorotetrahydro-1H-pyrrolizin-7a(5H)-yl)met hoxy)quinazolin-4-yl)-1-methyl-3,8-diazabicyclo[3.2.1]octane-8-carboxylate (Int 102b-2) was reacted in a similar manner as STEP C to give 4-(6,8-difluoro-2-(((2R,7aS)-2-fluorotetrahydro-1H-pyrrolizin-7a(5H)-yl)methoxy)-4-((1S,5R)-1-methyl-3,8-diazabicyclo[3.2.1]octan-3-yl)quinazolin-7-yl)-5-ethynylnaphthalen-2-ol, 2TFA. MS (ESI), m/z (M+H)⁺ 614.6. ¹H NMR (500 MHz, DMSO-de) S 10.84 (s, 1H), 10.27 (s, 1H), 9.53-9.10 (m, 2H), 7.93 (dd, J=8.3, 1.5 Hz, 1H), 7.71 (d, J=9.8 Hz, 1H), 7.57-7.36 (m, 3H), 7.08 (d, J=2.5 Hz, 1H), 5.57 (d, J=52.3 Hz, 1H), 4.59 (s, 2H), 4.44 (d, J=13.7 Hz, 1H), 4.32 (d, J=13.7 Hz, 1H), 4.23 (s, 1H), 3.80 (dd, J=60.0, 15.5 Hz, 4H), 3.59 (d, J=16.3 Hz, 2H), 2.69-2.53 (m, 2H), 2.42-2.27 (m, 1H), 2.26-1.94 (m, 6H), 1.87-1.69 (m, 1H), 1.53 (s, 3H).

Using methods similar to those described above for Example 102, Examples 103-105 were prepared.

| | | | [M + H]⁺ | Purification |
|---|---|---|---|---|
| Ex. | Structure | Compound Name | Found | conditions |
| Ex 103a | 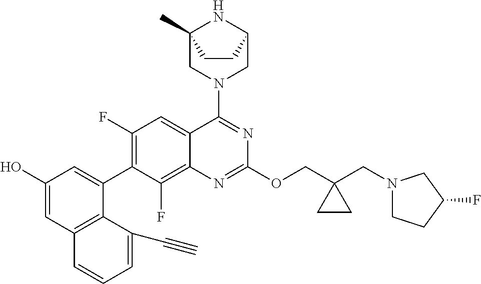 [SMILES: C#Cc1cccc2cc(O)cc(-c3c(F)cc4c(N5C[C@H]6CC[C@@](C)(C5)N6)nc(OCC5(CN6CC[C@@H](F)C6)CC5)nc4c3F)c12] | 4-(6,8-difluoro-2-((1-(((R)-3- fluoropyrrolidin-1- yl)methyl)cyclopropyl)methoxy)-4- ((1S,5R)-1-methyl-3,8- diazabicyclo[3.2.1]octan-3-yl)quinazolin- 7-yl)-5-ethynylnaphthalen-2-ol | 628 | Column R; Condition: 0.1% NH₄OH, 25% methanol |
| | Peak 1 | | | |
| Ex.103b | 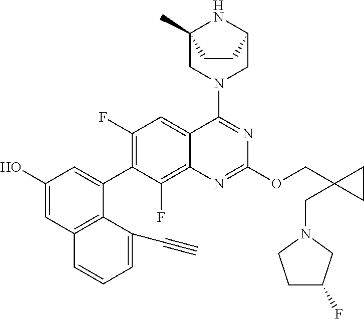 [SMILES: C#Cc1cccc2cc(O)cc(-c3c(F)cc4c(N5C[C@H]6CC[C@@](C)(C5)N6)nc(OCC5(CN6CC[C@@H](F)C6)CC5)nc4c3F)c12] | 4-(6,8-difluoro-2-((1-(((R)-3- fluoropyrrolidin-1- yl)methyl)cyclopropyl)methoxy)-4- ((1S,5R)-1-methyl-3,8- diazabicyclo[3.2.1]octan-3- yl)quinazolin-7-yl)-5-ethynylnaphthalen-2- ol, TFA | 628 | Column R; Condition: 0.1% NH₄OH, 25% methanol |
| | Peak 2 | | | |
| Ex 104 | 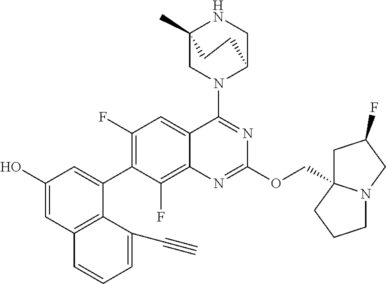 [SMILES: C#Cc1cccc2cc(O)cc(-c3c(F)cc4c(N5C[C@]6(C)CC[C@H]5CN6)nc(OC[C@@]56CCCN5C[C@H](F)C6)nc4c3F)c12] | 4-(6,8-difluoro-2-(((2R,7aS)-2- fluorotetrahydro-1H-pyrrolizin-7a(5H)- yl)methoxy)-4-((1S,4S)-4-methyl-2,5- diazabicyclo[2.2.2]octan-2-yl)quinazolin- 7-yl)-5-ethynylnaphthalen-2-ol | 614 | Column M; Condition: 0.1% NH₄OH, 40% isopropanol |
| Ex 105 | 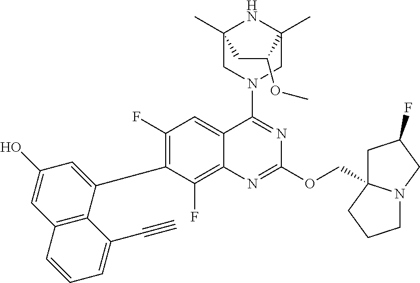 [SMILES: C#Cc1cccc2cc(O)cc(-c3c(F)cc4c(N5CC6(C)N[C@@](C)(C[C@H]6OC)C5)nc(OC[C@@]56CCCN5C[C@H](F)C6)nc4c3F)c12] | 4-(6,8-difluoro-2-(((2R,7aS)-2- fluorotetrahydro-1H-pyrrolizin-7a(5H)- yl)methoxy)-4-((1S,5R)-6-methoxy-1,5- dimethyl-3,8-diazabicyclo[3.2.1]octan-3- yl)quinazolin-7-yl)-5-ethynylnaphthalen- 2-ol | 658 | Column B; Condition: 0.1% NH₄OH, 25% isopropanol |
| Ex.105 | 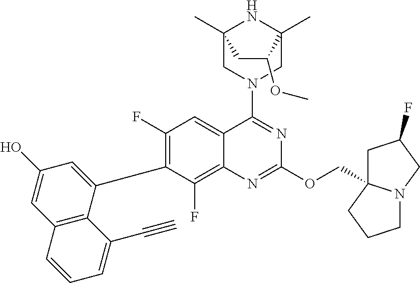 [SMILES: C#Cc1cccc2cc(O)cc(-c3c(F)cc4c(N5CC6(C)N[C@@](C)(C[C@H]6OC)C5)nc(OC[C@@]56CCCN5C[C@H](F)C6)nc4c3F)c12] | 4-(6,8-difluoro-2-(((2R,7aS)-2- fluorotetrahydro-1H-pyrrolizin-7a(5H)- yl)methoxy)-4-((1 S,5R)-6-methoxy-1,5-dimethyl-3,8- diazabicyclo[3.2.1]octan-3-yl)quinazolin- 7-yl)-5-ethy nylnaphthalen-2-ol, 2TFA | 658 | Column B; Condition: 0.1% NH₄OH, 25% isopropanol |
| Ex.240 | 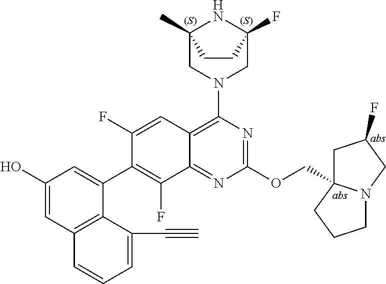 | 4-(6,8-difluoro-4-((18,5S)-1-fluoro-5- methyl-3,8-diazabicyclo[3.2.1]octan-3-yl)- 2-(((2R,7aS)-2-fluorot etrahydro-1H-pyrrolizin-7a(5H)- yl)methoxy)quinazolin-7-yl)-5- ethynylnaphthalen-2-ol | 632 | |
| Ex.241 | 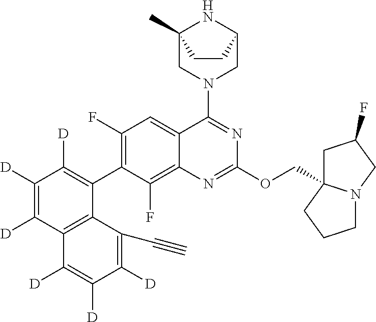 [SMILES: [2H]c1c([2H])c(C#C)c2c(-c3c(F)cc4c(N5C[C@H]6CC[C@@](C)(C5)N6)nc(OC[C@@]56CCCN5C[C@H](F)C6)nc4c3F)c([2H])c([2H])c([2H])c2c1[2H]] | 7-(8-ethynylnaphthalen-1-yl-2,3,4,5,6,7- d6)-6,8-difluoro-2-(((2R,7aS)-2- fluorotetrahydro-1H-pyrrolizin-7a(5H)- yl)methoxy)-4-((1S,5R)- 1-methyl-3,8-iazabicyclo[3.2.1]octan-3- yl)quinazoline, 2TFA | 604 | Column: AS-H, 21 × 250 mm, 5 um Condition: 20% MeOH w/ 0.1% NH4OH |
| | Peak 2 | | | |
| | Negishi coupling with Int-BZ-2_at | | | |
| | 70° C., 16 hr | | | |
| Ex.242a | 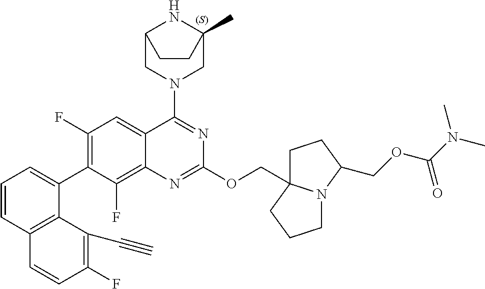 | (7a-(((7-(8-ethynyl-7- fluoronaphthalen-1-yl)-6,8- difluoro-4-((1S,5R)-1-methyl-3,8- diazabicyclo[3.2.1]octan-3-yl)quinazolin- 2-yl)oxy)methyl)hexahydro-1H- pyrrolizin-3-yl)methyl dimethylcarbamate | 699 | Column: WHELK-O1 (250 mm * 30 mm, 5 um); Condition: 0.1% NH3H2O IPA |
| | Peak 1 from SFC | | | |
| Ex.242b | 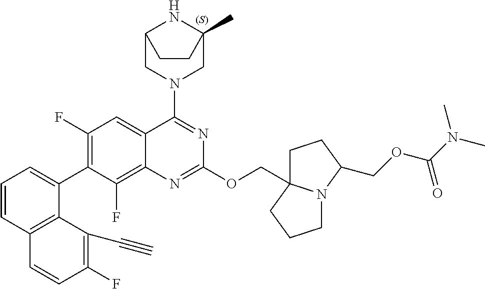 | (7a-(((7-(8-ethynyl-7-fluoronaphthalen-1- yl)-6,8-difluoro-4-((1S,5R)-1-methyl-3,8- diazabicyclo[3.2.1]octan-3-yl)quinazolin- 2-yl)oxy )methyl)hexahydro-1H- pyrrolizin-3-yl)methyl dimethylcarbamate | 699 | Column: WHELK-O1 (250 mm * 30 mm, 5 um); Condition: 0.1% NH3H2O IPA |
| | Peak 2 from SFC | | | |
| Ex.243a | 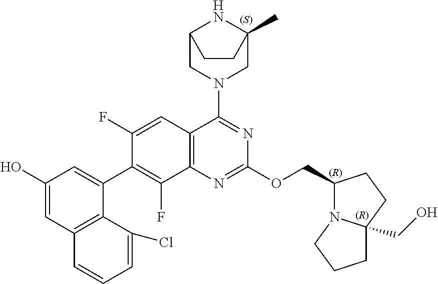 | 5-chloro-4-(6,8-difluoro-2-(((38,7aS)-7a- (hydroxymethyl)hexahydro-1H-pyrrolizin- 3-yl)methoxy)-4-((1S,5R)-1-methyl-3,8- diazabicyclo[3.2.1]octan-3-yl)quinazolin- 7-yl)naphthalen-2-ol | 636 | |
| | Peak 1 | | | |
| Ex. 243b | 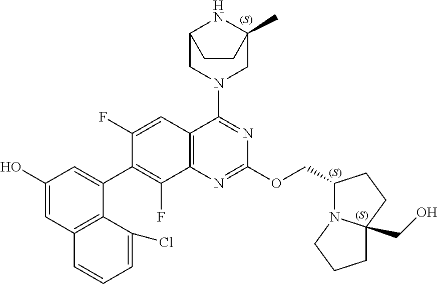 | 5-chloro-4-(6,8-difluoro-2-(((3S,7aS)- 7a-(hydroxymethyl)hexahydro-1H- pyrrolizin-3-yl)methoxy)-4-((1S,5R)-1- methyl-3,8-diazabicyclo[3.2.1]octan-3- yl)quinazolin-7-yl)naphthalen-2-ol | 636 | |
| | Peak 2 | | | |
| Ex.244 | 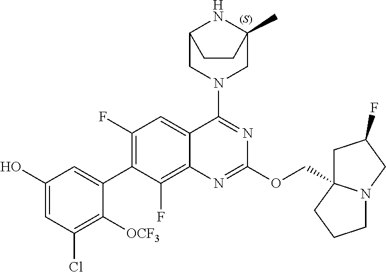 | 3-chloro-5-(6,8-difluoro-2-(((2RS,7aS)-2- fluorotetrahydro-1H-pyrrolizin-7a(5H)- yl)methoxy)-4-((1S,5R)-1-methyl-3,8- diazabicyclo[3.2.1]octan-3-yl)quina-zolin- 7-yl)-4-(trifluoromethoxy)phenol | 658 | |
| Ex.245a | 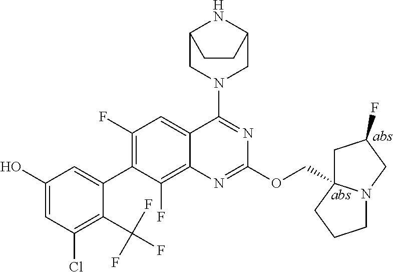 [SMILES: Oc1cc(Cl)c(C(F)(F)F)c(-c2c(F)cc3c(N4CC5CCC(C4)N5)nc(OC[C@@]45CCCN4C[C@H](F)C5)nc3c2F)c1] | 3-(4-((1R,5S)-3,8-diazabicyclo[3.2.1] octan-3-yl)-6,8-difluoro-2-(((2R,7aS)- 2-fluorotetrahydro-1H-pyrrolizin-7a(5H)- yl)methoxy)quinazolin-7-yl)-5-chloro-4- (trifluoromethyl)phenol | 628 | Column A; Condition 0.1% NH3H2O IPA |
| | Peak 1 from SFC | | | |
| Ex.246a | 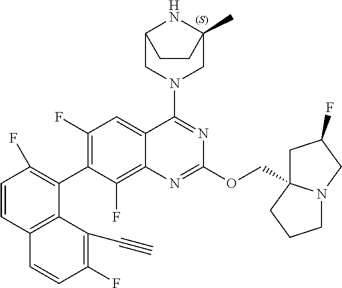 | 7-(8-ethynyl-2,7-difluoronaphthalen-1-yl)- 6,8-difluoro-2-(((2R,7aS)-2- fluorotetrahydro-1H-pyrrolizin-7a(5H)- yl)methoxy)-4-((1S,5R)-1-methyl-3,8- diazabicyclo[3.2.1] octan-3-yl)quinazoline | 634 | Column: (s,s) WHELK-O1 (250 mm * 30 mm, 5 um) Condition 0.1% NH₃H₂O EtOH |
| | Peak 1 after SFC | | | |
| Ex.246b | 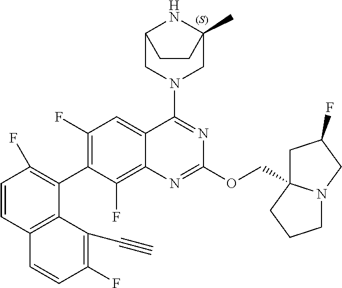 | 7-(8-ethynyl-2,7-difluoronaphthalen-1-yl)- 6,8-difluoro-2-(((2R,7aS)-2- fluorotetrahydro-1H-pyrrolizin-7a(5H)- yl)methoxy)-4-((1S,5R)-1-methyl-3,8- diazabicyclo[3.2.1] octan-3-yl)quinazoline | 634 | Column: (s,s) WHELK-O1 (250 mm * 30 mm, 5 um) Condition 0.1% NH₃H₂O EtOH |
| | Peak 2 after SFC | | | |

Ex. 106 in the table below was prepared via a similar procedure to Ex. 89.

Ex. 247a and 247b were made using a similar procedure as Ex. 106 using the piperazine intermediates described in other sections.

| | | | [M + H]⁺ |
|---|---|---|---|
| Ex | Structure | Compound Name | Found |
| Ex 106a | 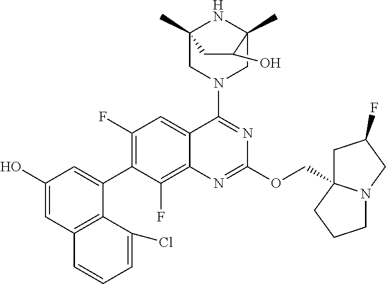 [SMILES: C[C@]12CC(O)[C@@](C)(CN(c3nc(OC[C@@]45CCCN4C[C@H](F)C5)nc4c(F)c(-c5cc(O)cc6cccc(Cl)c56)c(F)cc34)C1)N2] | (1S,5R)-3-(7-(8-chloro-3-hydroxynaphthalen-1-yl)-6,8- difluoro-2-(((2R,7aS)-2-fluorotetrahydro-1H-pyrrolizin- 7a(5)-yl)methoxy)quinazolin-4-yl)-1,5-dimethyl-3,8- diazabicyclo[3.2.1]octan-6-ol | 654 |
| | Peak 1 | | |
| Ex. 106b | 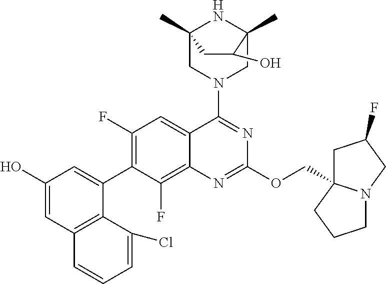 [SMILES: C[C@]12CC(O)[C@@](C)(CN(c3nc(OC[C@@]45CCCN4C[C@H](F)C5)nc4c(F)c(-c5cc(O)cc6cccc(Cl)c56)c(F)cc34)C1)N2] | (1S,5R)-3-(7-(8-chloro-3-hydroxynaphthalen- 1-yl)-6,8-difluoro-2-(((2R,7aS)-2-fluorotetrahydro- 1H-pyrrolizin-7a(5H)-yl)methoxy) quinazolin-4-yl)-1,5-dimethyl-3,8-diazabicyclo [3.2.1]octan-6-ol, 2TFA | 654 |
| | Peak 2 | | |
| Ex. 247b | 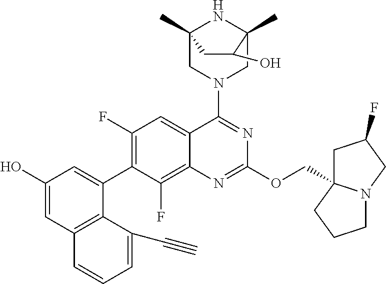 [SMILES: C#Cc1cccc2cc(O)cc(-c3c(F)cc4c(N5C[C@]6(C)CC(O)[C@@](C)(C5)N6)nc(OC[C@@]56CCCN5C[C@H](F)C6)nc4c3F)c12] | (1S,5R)-3-(7-(8-ethynyl-3-hydroxynaphthalen- 1-yl)-6,8-difluoro-2-(((2R,7aS)-2-fluorotetrahydro- 1H-pyrrolizin-7a(5H)-yl)methoxy)quinazolin-4-yl)-1,5- dimethyl-3,8-diazabicyclo[3.2.1 ]octan-6-ol, 2TFA | 644.3 |
| | Peak 2 | | |
| Ex. 247a | 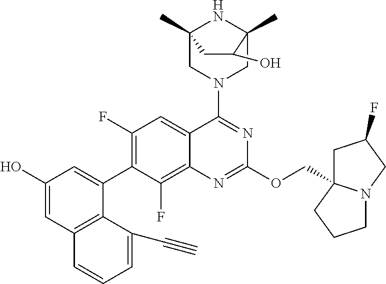 [SMILES: C#Cc1cccc2cc(O)cc(-c3c(F)cc4c(N5C[C@]6(C)CC(O)[C@@](C)(C5)N6)nc(OC[C@@]56CCCN5C[C@H](F)C6)nc4c3F)c12] | (1S,5R)-3-(7-(8-ethynyl-3-hydroxynaphthalen-1-yl)-6,8- difluoro-2-(((2R,7aS)-2-fluorotetrahydro-1H-pyrrolizin- 7a(5H)-yl)methoxy)quinazolin-4-yl)-1,5-dimethyl-3,8- diazabicyclo[3.2.1]octan-6-ol, 2TFA | 644.3 |
| | Peak 1 | | |
| 248b | 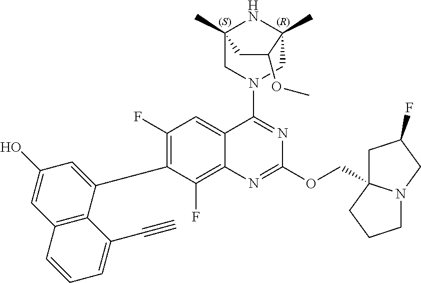 | 4-(6,8-difluoro-2-(((2R,7aS)-2-fluorotetrahydro-1H- pyrrolizin-7a(5H)-yl)methoxy)-4-((1S,5R)-6-methoxy- 1,5-dimethyl-3,8-diazabicyclo[3.2.1]octan-3- yl)quinazolin-7-yl)-5-ethynylnaphthalen-2-ol, 2TFA | 657 |
| | Peak 2 from SFC | | |
| 248a = Ex. 105 | 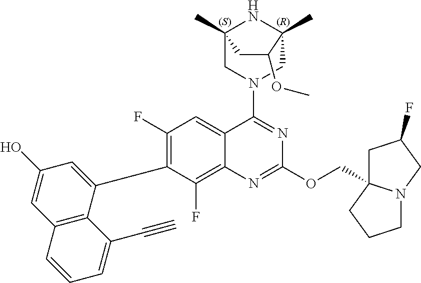 | 4-(6,8-difluoro-2-(((2R,7aS)-2-fluorotetrabydro- 1H-pyrrolizin-7a(5H)-yl)methoxy)-4-((1S,5R)-6- methoxy-1,5-dimethyl-3,8-diazabicyclo[3.2.1]octan-3- yl)quinazolin-7-yl)-5-ethynylnaphthalen-2-ol, 2TFA | 657 |
| | Peak 1 from SFC | | |
| Ex.249a | 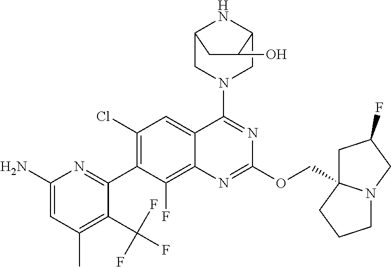 [SMILES: Cc1cc(N)nc(-c2c(Cl)cc3c(N4CC5CC(O)C(C4)N5)nc(OC[C@@]45CCCN4C[C@H](F)C5)nc3c2F)c1C(F)(F)F] | (IR,SR,6R)-3-(7-(6-amino-4-methyl-3-(trifluoro methyl)pyridin-2-yl)-6-chloro-8-fluoro-2-(((2RS,7aSR)- 2-fluorotetrahydro-1H-pyrrolizin-7a(5H)- yl)methoxy)quinazolin-4-yl)-3,8- diazabicyclo[3.2.1]octan-6-ol | 640 |
| | Peak 1 | | |
| Ex.249b | 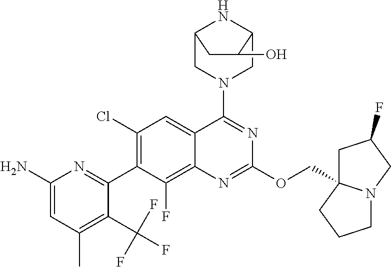 [SMILES: Cc1cc(N)nc(-c2c(Cl)cc3c(N4CC5CC(O)C(C4)N5)nc(OC[C@@]45CCCN4C[C@H](F)C5)nc3c2F)c1C(F)(F)F] | (1R,5R,6R)-3-(7-(6-amino-4-methyl-3-(trifluoro methyl)pyridin-2-yl)-6-chloro-8-fluoro-2-(((2RS,7aSR)- 2-fluorotetrahydro-1H-pyrrolizin-7a(5H)- yl)methoxy)quinazolin-4-yl)-3,8- diazabicyclo[3.2.1]octan-6-ol | 640 |
| | Peak 2 | | |

Example 250: Synthesis of 4-U1R,5S)-3,8-diazabicyclo[3.2.1]octan-3-yl)-7-(3.6-dimethyl-5-(trifluoromethyl)-1H-pyrazolo[3.4-b]pyridin-4-yl)-6A4-fluoro-2-(((2R,7aS)-2-fluoroterhyro-1H-pyrizin-7a(5H)methox)quinazoline (Ex. 250a and 250b

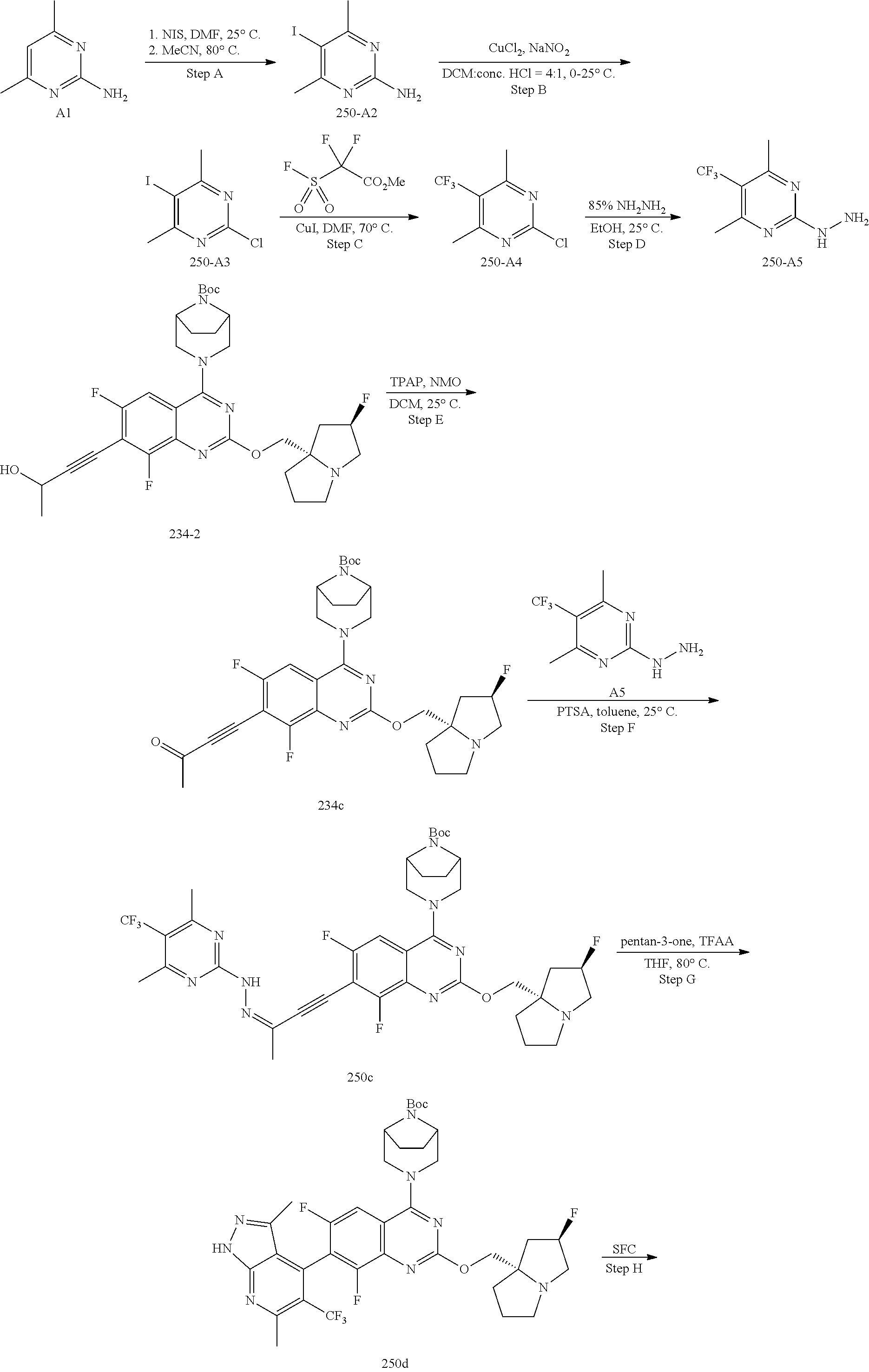

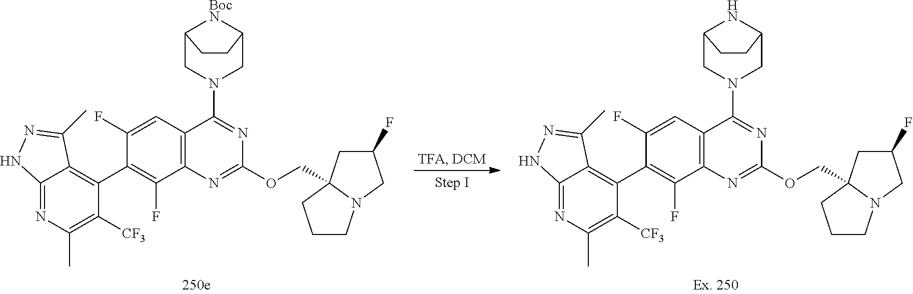

### Step A: 5-iodo-4,6-dimethyrnrmidin-2-amine (Int-250-A2)

To a solution of 4,6-dimethylpyrimidin-2-amine (10 g, 81 mmol) in DMF (100 mL) was added N₁S (36.5 g, 162 mmol) at 25° C., and the reaction was stirred at 25° C. for 16 h. LCMS showed the starting material was consumed and desired MS was formed. The reaction mixture was diluted with EtOAc (500 mL), and washed with H₂O (50 mL×3) and brine (50 mL×3). The organic phase was concentrated in vacuo, and the residue was purified by flash silica gel chromatography 80 g, Column, Pet, ether/EtOAc=2/l to give 5-iodo-4,6-dimethylpyrimidin-2-amine as yellow solid. ¹HNMR showed the product was a mixture. The product was dissolved in MeCN (100 mL), and the mixture was stirred at 60° C. for 16 h. The resulting mixture was concentrated in vacuo to give 5-iodo-4,6-dimethylpyrimidin-2-amine MS (ESI): m/z (M+H)⁺ 250. ¹H NMR (400 MHz, DMSO) δ 6.76-6.57 (m, 2H), 2.46 (s, 6H).

### Step B: 2-chloro-5-iodo-4,6-dimethylovrimidine (Int-250-A3)

To a solution of 5-iodo-4,6-dimethylpyrimidin-2-amine (Int-250-A2) (3 g, 12.05 mmol) in DCM (30 mL) and conc. HCl (10 mL) was added copper(II) chloride (4.86 g, 36.1 mmol) at 25° C., and the reaction was stirred at 25° C. for 10 min. After cooled to 0° C., sodium nitrite (2.493 g, 36.1 mmol) in water (5 mL) was added dropwise to the reaction mixture, and the resulting mixture was allowed to warm to room temperature (25° C.) and stirred at 25° C. for 2 h. The reaction mixture was continued to stirred at 25° C. for 1 h. The reaction mixture was quenched with saturated Na₁HCO₃(50 mL), and extracted with EtOAc (100 mL×2). The combined organic phase was dried over anhydrous Na₂SO₄, filtered and concentrated in vacuo. The residue was purified by flash silica gel chromatography 24 g column, Pet, ether/EtOAc=10/1) to give 2-chloro-5-iodo-4,6-dimethylpyrimidine. MS (ESI): m/z (M+H)⁺ 268. ¹H NMR (400 MHz, CDCl₃) δ 2.65 (s, 6H).

### Step C: 2-chloro-4,6-dimethyl-5-(trifluoromethyl)pyrimidine (Int-250-A4)

To a solution of 2-chloro-5-iodo-4,6-dimethylpyrimidine (Int-250-A3) (500 mg, 1.862 mmol) and copper(I) iodide (1064 mg, 5.59 mmol) in DMF (5 mL) was added methyl 2,2-difluoro-2-(fluorosulfonyl)acetate (1073 mg, 5.59 mmol) at 25° C., and the reaction was stirred at 70° C. for 12 h under N₂. The reaction mixture was filtered, and the filtered was washed with EtOAc (20 mL). The mixture was diluted with water (10 mL), and then extracted with EtOAc (50 mL rex 3). The combined organic layer was washed with brine (5 mL×3), dried over anhydrous Na₂SO₄, filtered and concentrated in vacuo. The residue was purified by flash silica gel chromatography 4 g column, Pet, ether/EtOAc=10/1) to give 2-chloro-4,6-dimethyl-5-(trifluoromethyl)pyrimidine. MS (EST): m/z (M+H)⁺ 211.

### Step D: 2-hydrazineyl-4,6-dimethyl-5-(trifluoromethyl)pyrimidine (Int-250-A5)

To a solution of 2-chloro-4,6-dimethyl-5-(trifluoromethyl)pyrimidine (Int-250-A4)(80 mg, 0.380 mmol) in ethanol (1 mL) was added hydrazine (0.014 mL, 0.380 mmol) (85% aqueous solution), and the reaction mixture was stirred at 25° C. for 5 min. The reaction mixture was extracted with EtOAc (50 mL), and the organic phase was concentrated in vacuo to give 2-hydrazineyl-4,6-dimethyl-5-(trifluoromethyl)pyrimidine, which was used to next step without further purification. MS (EST): m/z (M+H)⁺ 207.

### Step E: Tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate (Int-234c)

To a solution of tert-butyl (1R,5S)-3-(6,8-difluoro-2-(((2R,7aS)-2-fluorotetrahydro-1H-pyrrolizin-7a(5H)-yl)methoxy)-7-(3-hydroxybut-1-yn-1-yl)quinazolin-4-yl)-3,8-diazabicyclo[3.2.1]octane-8-carboxylate (Int-234-2) (1.1 g, 1.828 mmol) in DCM (20 mL) was added NMO (0.857 g, 7.31 mmol) and TPAP (0.096 g, 0.274 mmol), and the mixture was stirred at 25° C. for 12 h. The reaction mixture was filtered and the filtered cake was washed with EtOAc (30 mL). The organic phase was concentrated in vacuum, and the residue was purified by reversed MPLC 80 g, C18, 20-35 pm, Eluent of 0%˜ 50% MeCNH₂O (0.5% TFA) gradient to give tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate. MS (ESI): m/z (M+H)⁺ 600.

### Step F: Tert-butyl (1R,5S)-3-(7-((Z)-3-(2-(4,6-dimethyl-5-(trifluoromethyl)pyrimidin-2-y)hydrazineyldene)but-1-yn-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250c)

To a solution of tert-butyl (1R,5S)-3-(6,8-difluoro-2-(((2R,7aS)-2-fluorotetrahydro-1H-pyrrolizin-7a(5H)-yl)methoxy)-7-(3-oxobut-1-yn-1-yl)quinazolin-4-yl)-3,8-diazabicyclo[3.2.1]octane-8-carboxylate (Int-234c) (270 mg, 0.450 mmol) in toluene (3 mL) was added 2-hydrazineyl-4,6-dimethyl-5-(trifluoromethyl)pyrimidine (Int-250-A5) (93 mg. 0.450 mmol) and p-toluenesulfonic acid monohydrate (17.13 mg, 0.090 mmol), and the reaction mixture was stirred at 25° C. for 10 h. The reaction mixture was concentrated in vacuo, and the residue was purified by preparative TLC plate (SiO₂, DCM/MeOH=10/1) to give tert-butyl (1R,5S)-3-(7-((Z)-3-(2-(4,6-dimethyl-5-(trifluoromethyl)pyrimidin-2-yl)hydrazineyldene)but-1-yn-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI): m/z (M+H)⁺ 788.

### Step G: Tert-butyl (1R,5S)-3-(7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250d)

To a solution of tert-butyl (1R,5S)-3-(7-((Z)-3-(2-(4,6-dimethyl-5-(trifluoromethyl)pyrimidin-2-yl)hydrazineyldene)but-1-yn-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250c) (170 mg, 0.173 mmol) and 3-pentanone (223 mg, 2.59 mmol) in THF (20 mL) was added TFAA (0.268 mL, 1.899 mmol), and the mixture was stirred at 80° C. for 4 h. The reaction mixture was concentrated in vacuo, and the residue was purified by preparative TLC plate (SiO₂. DCM/MeOH=10/1) to give tert-butyl (1R,5S)-3-(7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI): m/z (M+H)⁺ 747.

### Step H: Tert-butyl (1R,5S)-3-(7-(3.6-dimethyl-5-(trifluoromethyl)- H-pyrazolo[3.4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250e)

The racemic tert-butyl (1R,5S)-3-(7-(3.6-dimethyl-5-(trifluoromethyl)-1H-pyrazolo[3,4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250d) (160 mg. 0.214 mmol) was separated by preparative SFC (Column (s,s) WHELK-O1 (250 mm*30 mm, 5 um) Condition 0.1% NH₃H₂O IPA Begin B 30% End B 30% Gradient Time (min) 100% B Hold Time (min) FlowRate (mL/min) 80 Injections 60) to give tert-butyl (1R,5S)-3-(7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250e_P1, the first eluting isomer) as white solid and tert-butyl (1R,5S)-3-(7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250e_P2: the second eluting isomer. MS (ESI): m/z (M+H)⁺ 747.

### Step 1: 4-((1R,5S)-3,8-diazabicyclo[3.2.1]octan-3-yl)-7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazoline (Ex. 250)

To a solution of tert-butyl (1R,5S)-3-(7-(3.6-dimethyl-5-(trifluoromethyl)-1H-pyrazolo[3,4-b]pyridin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-250e) (42 mg, 0.056 mmol) in DCM (1 mL) was added TFA (0.25 mL, 3.24 mmol), and the mixture was stirred at 25° C. for 1 h. The reaction mixture was concentrated in vacuo, and the residue was purified by preparative HPLC (Column: Boston Green ODS 150*30 mm*5 um; Condition: water (0.01% TFA)-ACN FlowRate (mL/min) 25 to give 4-((1R,5S)-3,8-diazabicyclo[3.2.1]octan-3-yl)-7-(3.6-dimethyl-5-(trifluoromethyl)-1H-pyrazolo[3.4-b]pyridin-4-yl)-6,8-difluoro-2-(((2R,7aS)-2-fluorotetrahydro-1H-pyrrolizin-7a(5H)-yl)methoxy)quinazoline. Ex. 250a_P1 ¹H NMR (400 MHz, MeOD) δ 7.83 (d, J=8.8 Hz, 11H), 5.70-5.52 (m, 1H), 4.76-4.62 (m, 4H), 4.27 (br s, 2H), 4.09-3.80 (m, 5H), 3.49 (dt, J=5.9, 10.6 Hz, 1H), 2.91 (d, J=2.3 Hz, 3H), 2.83-2.53 (m, 2H), 2.51-2.42 (m, 1H), 2.41-2.31 (m, 2H), 2.28-2.10 (M, 5H), 1.89 (s, 3H). MS (ESI): m/z (M+H)⁺ 647. The procedure of Ex.250b_P2 was similar as Peak 1_P1-spectrum data: Ex.250b_P2 ¹H NMR (400 MHz. MeOD) δ 7.83 (d, J=8.7 Hz, 1H), 5.69-5.52 (m, 1H), 4.78-4.60 (m, 4H), 4.27 (br s, 2H), 4.10-3.77 (m, 5H), 3.49 (dt, J=6.0, 10.6 Hz, 1H), 2.91 (d, J=2.1 Hz, 3H), 2.84-2.53 (M. 2H), 2.52-2.43 (m, 1H), 2.41-2.29 (m, 2H), 2.28-2.12 (m, 5H), 1.89 (s, 3H). MS (ESI): m/z (M+H)⁺ 647.

### Ex 251: Synthesis of 4-((1R,5S)-38-diazabicyclo[3.2.1]octan-3-yl)-7-(1,5-dimethyl-6-(trifluoromethyl)-1H-indazol-7-yl)-68-difluoro-2-(((2R,7aS)-2-fluorotetrahydro-1H-pyrrolizin-7a(5H)-yl)methoxy)quinazoline (Ex. 251a) and 4-((1R,5)-3,8-diazabicyclo[3.2.1]octan-3-yl)-7-(2,5-dimethyl-6-(trifluoromethyl)-2H-indazol-7-yl)-68-difluoro-2-(((2R,7aS)-2-fluorotetrahydro-1H-pyrrolizin-7a(5H)-yl)methoxy)quinazoline (Ex. 251b)

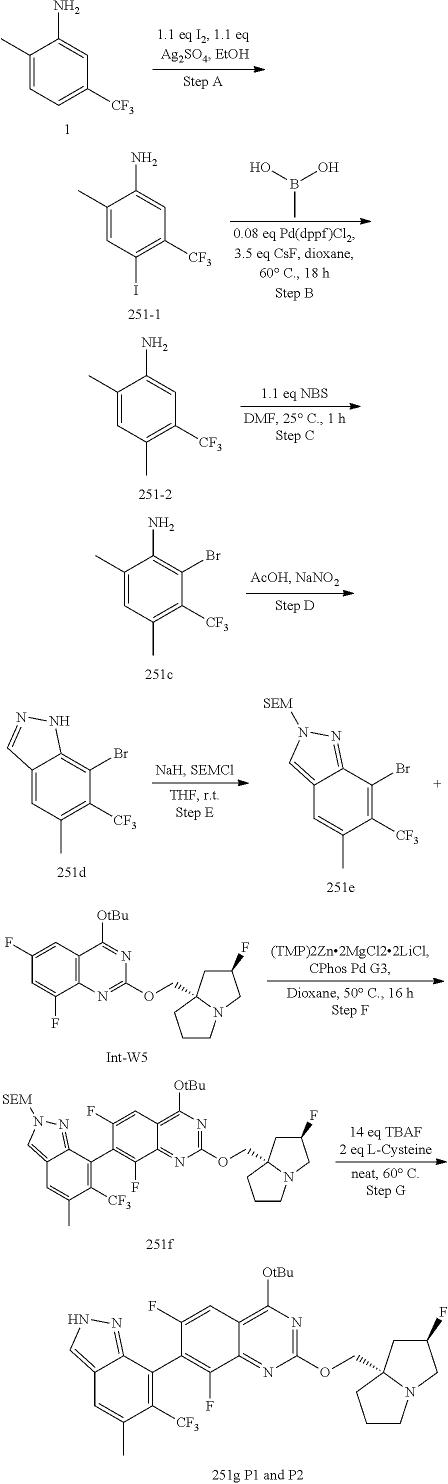

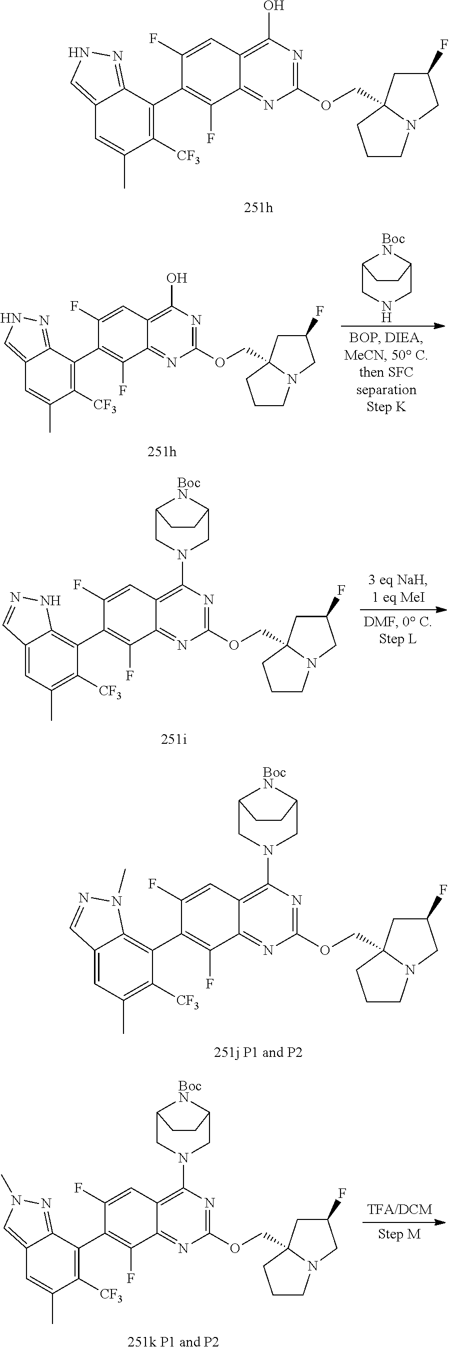

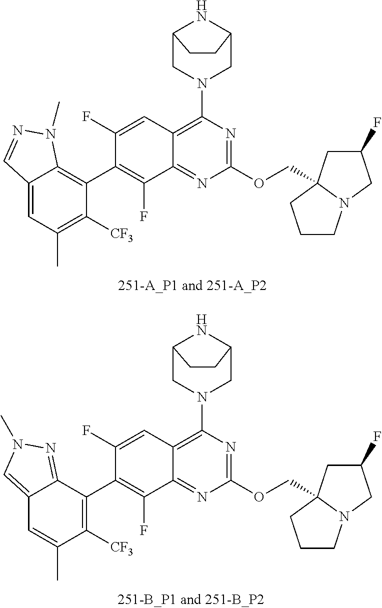 [SMILES: Cc1cc2cnn(C)c2c(-c2c(F)cc3c(N4CC5CCC(C4)N5)nc(OC[C@@]45CCCN4C[C@H](F)C5)nc3c2F)c1C(F)(F)F ; Cc1cc2cn(C)nc2c(-c2c(F)cc3c(N4CC5CCC(C4)N5)nc(OC[C@@]45CCCN4C[C@H](F)C5)nc3c2F)c1C(F)(F)F]

### Steu A: 4-iodo-2-methyl-5-(trifluoromethyl)aniline (Int-251-1)

A solution of 2-methyl-5-(trifluoromethyl)aniline (14.87 g, 85 mmol) in ethanol (225 mL) was added to a suspension of silyler sulfate (26.5 g, 85 mmol) and I₂ (21.55 g, 85 mmol) in ethanol (380 mL). The mixture was stirred at 25° C. for 1 h under nitrogen atmosphere. The mixture was filtered through a pad of a CELITE, and washed with additional ethanol (200 mL). The filtrate was concentrated in vacuo and the residue was diluted with EtOAc (300 mL), then basified with sat. NaHCO₃(aq., 100 mL). The organic layer was washed with brine (20 mL×3), dried over Na₂SO₄ and concentrated in vacuo. The residue was purified by flash silica gel chromatography Column Silica-CS 80 g. Eluent of 0-10% EtOAc/Pet, ether to give 4-iodo-2-methyl-5-(trifluoromethyl)aniline. MS (ESI): m/z (M+H)⁺: 301. ¹H NMR (400 MHz, CDCl₃) S: 7.63 (s, 1H), 6.96 (s, 1H), 2.15 (s, 3H).

### Step B: 2,4-dimethyl-5-(trifluoromethyl)aniline (Int-251-2)

To a mixture of 4-iodo-2-methyl-5-(trifluoromethyl)aniline (Int-251-1) (10.93 g, 36.3 mmol) in dioxane (110 mL) was added CsF (38.6 g, 254 mmol), methylboronic acid (13.04 g, 218 mmol) and PdCl₂(dppf) (2.125 g, 2.90 mmol) at 25° C. The mixture was stirred at 60° C. for 16 h under N₂ atmosphere. LCMS showed the reaction was finished. The reaction mixture was diluted with EtOAc (200 mL), washed with H₂O (50 mL) and brine (50 mL). The organic layer was dried over sodium sulfate and concentrated in vacuum. The crude product was purified by flash silica gel chromatography 120 g Flash column, Eluent of 0-5% EtOAc/Pet, ether gradient, dry load to give 2,4-dimethyl-5-(trifluoromethyl)aniline. MS (ESI): m/z (M+H)⁺ 190.. ¹H NMR (400 MHz, CDCl₃) S: 6.95 (s, 1H), 6.91 (s, 1H), 3.61 (br s, 2H), 2.34 (d, J=1.7 Hz., 3H), 2.17 (s, 3H).

### Step C: 2-bromo-4,6-dimethyl-3-(trifluoromethyl)aniline (Int-251c)

To a solution of 2,4-dimethyl-5-(trifluoromethyl)aniline (Int-251-2) (3.05 g, 16.12 mmol) in DMF (30 mL) was added NBS (3.16 g, 17.73 mmol). The reaction was stirred at 25° C. for 1 h. The reaction mixture was diluted with EtOAc (2(X) mL), and washed with Na₂SO₃ (aq., 30 mL ×2). The organic layer was washed with brine (50 mL), dried over Na₂SO₄ and concentrated in vacuo to give the crude product, which was purified by flash silica gel chromatography 80 g column, Eluent of 3% ethyl acetate in petroleum ether gradient to give 2-bromo-4,6-dimethyl-3-(trifluoromethyl)aniline. MS (ESI): m/z (M+H)⁺ 268, 270, 1H NMR (400 MHz, CDCl₃) &: 6.91 (s, 1H), 4.27 (br s, 2H), 2.42 (q, J=4.3 Hz, 3H), 2.24 (s, 3H).

### Step D: 7-bromo-5-methyl-6-(trifluoromethyl)-1H-indazole (Int-251d)

To a solution of 2-bromo-4,6-dimethyl-3-(trifluoromethyl)aniline (Int-251c) (4.09 g, 15.26 mmol) in AcOH (41 mL) was added a solution of sodium nitrite (1,368 g, 19.83 mmol) in water (4.0 mL). The reaction was stirred at 25° C. for 16 h. The reaction mixture was diluted with H₂O (200 mL). The resulting mixture was filtered and the filtered cake was washed with H₂O (30 mL). The solid was collected and diluted with EtOAc (200 mL), dried over Na₂SO₄, and concentrated in vacuum to give the crude product, which was purified by flash silica gel chromatography 40 g column, Eluent of 0-12% EtOAc/Pet, ether gradient, dry load to give 7-bromo-5-methyl-6-(trifluoromethyl)-1H-indazole. MS (ESI): m/z (M+H)⁺ 278, 280. ¹H NMR (400 MHz, CDCl₃) S:10.32 (br s, 1H), 8.07 (s, 1H), 7.54 (s, 1H), 2.56 (q, J=2.8 Hz, 3H).

### Step E: 7-bromo-5-methyl-6-(trifluoromethyl)-1-((2-(trimethylsilyl)ethoxy)methyl)-2H-indazole (Int-251e_P1)

To a solution of 7-bromo-5-methyl-6-(trifluoromethyl)-1H-indazole (Int-251d) (2.04 g, 7.31 mmol) in THF (20 mL) was added NaH (0.614 g, 15.35 mmol) (60% w/w) at 0° C. and the mixture was stirred at 0° C. for 20 min. Then SEM-C₁ (2.074 mL, 11.70 mmol) was added to the above solution. The mixture was stirred at 25° C. for 10 min. The reaction mixture was quenched with NH₄Cl (50 mL), diluted with EtOAc (100 mL) and separated. The organic layer was washed with brine (30 mL), dried over Na₂SO₄, filtered and concentrated in vacuo. The residue was purified by flash silica gel chromatography (12 g), Eluent of 0-5% EtOAc/Pet, ether to give 7-bromo-5-methyl-6-(trifluoromethyl)-1-((2-(trimethylsilyl)ethoxy)methyl)-1H-indazole (Int-251e_P2: the second eluting isomer) and 7-bromo-5-methyl-6-(trifluoromethyl)-2-((2-(trimethylsilyl)ethoxy)methyl)-2H-indazole (Int-251e_P1: the first eluting isomer). MS (ESI): m/z (M+H)⁺ 409, 411. P1: ¹H NMR (400 MHz, CDCl₃) S: 7.97 (s, 1H), 7.55 (s, 1H), 6.13 (s, 2H), 3.48-3.54 (m, 2H), 2.60 (d, J=3.9 Hz, 3H), 0.84 (d, J=3.0 Hz, 2H), -0.10 (s, 9H). P2: ¹H NMR (400 MHz, CDCl₃) S: 8.19 (s, 1H), 7.54 (s, 1H), 5.81 (s, 2H), 3.65-3.70 (m, 2H), 2.60 (q,J=2.8 Hz, 3H), 0.94-1.01 (m, 2H), 0.00 (s, 9H).

### Step F: 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-14(2-(trimethylsilyl)ethoxy)methyl)-2H-indazol-7-yl)quinazoline (Int-251f-P1)

A solution of 4-(tert-butoxy)-6,8-difluoro-2-(((2R,7aS)-2-fluorotetrahydro-1H-pyrrolizin-7a(5H)-yl)methoxy)quinazoline (Int-AI10) (700 mg, 1.770 mmol) in (TMP)₂Zn-2MgCl₂LiCl (16.09 mL, 5.31 mmol) (0.33 M in THF) was stirred at 50° C. for 2 h under N₂ atmosphere. Then to the reaction mixture was added a solution of 7-bromo-5-methyl-6-(trifluoromethyl)-1-((2-(trimethylsilyl)ethoxy)methyl)-2H-indazole (Int-252e_P1) (800 mg, 1.954 mmol) and CPhos Pd G3 (143 mg, 0.177 mmol) in 1.4-dioxane (16 mL) in glove box. The resulting mixture was stirred at 50° C. for 16 h. The reaction mixture was diluted with EtOAc (100 mL), quenched with NaHCO₃(aq., 30 mL) and stirred for 10 min. The mixture was filtered and the filtrate was dried over Na₂SO₄, filtered and the solvent was evaporated under reduced pressure to give the crude product, which was purified by flash silica gel chromatography 12 g column, Eluent with 30% ethyl acetate in petroleum ether gradient to give 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-1-((2-(trimethylsilyl)ethoxy)methyl)-2H-indazol-7-yl)quinazoline. MS (ESI): m/z (M+H)⁺ 724.

### Step G: 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazoline (Int-251g Pt and Int-251g_P2) rac-6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazolin-4-ol (Int-251h racemic)

A solution of 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-2-((2-(trimethylsilyl)ethoxy)methyl)-2H-indazol-7-yl)quinazoline (Int-251g_P1) (570 mg, 0.787 mmol) and L-cysteine (191 mg, 1.575 mmol) in TBAF (11 mL, 11.02 mmol) (1 M in THF) was stirred at 60° C. for 16 h under N₂ atmosphere. The reaction mixture was diluted with EtOAc (10 mL), washed with aqueous NH₄Cl (2 mL×3) and brine (2 mL). The organic layer was dried over Na₂SO₄, filtered and concentrated in vacuum. The residue was purified by flash silica gel chromatography 12 g column, Eluent of 3% ethyl acetate in petroleum ether gradient to give 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazoline (Int-251g_P1: the first eluting isomer) and 4-(tert-butoxy)-6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazoline (Int-251g_P2: the second eluting isomer) as light yellow solid and rac-6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazolin-4-ol (Int-252h_racemic): the third eluting isomer) as light yellow solid. 251g_P1&P2: MS (ESI): m/z (M+H)⁺ 594, 251h: MS (ESI): m/z (M+H)⁺ 538.

### Step K: Tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazolin-4-yl)-3,8-diazabicyclo[3.2.1]octane-8-carboxylate (Int-251i_P1 and Int-251i_P2)

A solution of 6.8-difluoro-2-(((2R,7aS)-2-fluorotetrahydro-1H-pyrrolizin-7a(5H)-yl)methoxy)-7-(5-methyl-6-(trifluoromethyl)-2H-indazol-7-yl)quinazolin-4-ol (Int-252h) (100 mg. 0.186 mmol) and BOP (165 mg, 0.372 mmol) in MeCN (2 mL) was stirred at 20° C. for 10 min, then tert-butyl (1R,5S)-3,8-diazabicyclo[3.2.]octane-8-carboxylate (79 mg, 0.372 mmol) and N-ethyl-N-isopropylpropan-2-amine (72.1 mg, 0.558 mmol) was added in sequence. The resulting mixture was stirred at 50° C. for 16 h. The reaction was concentrated in vacuo, and the residue was purified with flash silica gel chromatography column (4 g), Eluent of 0-90% EtOAc/Pet, ether gradient to give tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-1H-indazol-7-yl)quinazolin-4-yl)-3.8-diazabicyclo[3.2.1]octane-8-carboxylate as a yellow solid. MS (ESI): m/z (M+H)⁺ 732. The racemic tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-1H-indazol-7-yl)quinazolin-4-yl)-3,8-diazabicyclo[3.2.1]octane-8-carboxylate (90 mg, 0.123 mmol) was separated by preparative SFC (Instrument SFC-11 Method Column DAICEL CHIRALPAK IG (250 mm*30 mm, 10 um) Condition 0.1% NH₃H₂O EtOH Begin B 40% End B 40% Gradient Time (min) 100% B Hold Time (min) to give tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-1H-indazol-7-yl)quinazolin-4-yl)-3,8-diazabicyclo[3.2.1]octane-8-carboxylate (Int-251i_P1: the first eluting isomer) and tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-1H-indazol-7-yl)quinazolin-4-yl)-3,8-diazabicyclo[3.2.1]octane-8-carboxylate (Int-251i_P2: the second eluting isomer).

### Step L: Tert-butyl (1R5S)-3-(7-(1,5-dimethyl-6-(trifluoromethyl)-1H-indazol-7-yl)-6,8-difluoro-2-((2R,7aS)-2-fluorotetrahydro-1H-pyrrolizin-7a(5H)-yl)methoxy)quinazolin-4-yl)-3,8-diazabicyclo[3.2.1]octane-8-carboxylate (251j_P1) & tert-butyl (0R,5S)-3-(7-(2,5-dimethyl-6-(trifluoromethyl)-2H-indazol-7-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate(251k_P1)

To a stirred solution of tert-butyl (1R,5S)-3-(6,8-difluoro-2-(((2R,7aS)-2-fluorotetrahydro-1H-pyrrolizin-7a(5H)-yl)methoxy)-7-(5-methyl-6-(trifluoromethyl)-1H-indazol-7-yl)quinazolin-4-yl)-3,8-diazabicyclo[3.2.1]octane-8-carboxylate (Int-251i) (22 mg, 0.030 mmol) in DMF (0.8 mL) was added NaH (4.81 mg, 0.120 mmol) at 0° C. The reaction was stirred at 0° C. for 5 min, then Mel (1.880 μl, 0.030 mmol) was added, the reaction was stirred at 0° C. for 2 min under N₂. The reaction mixture was diluted with EtOAc (5 mL), quenched with NH₄Cl (aq., 0.5 mL), washed with brine (0.5 mL×4), the organic layer was concentrated in vacuo. The residue was purified by preparative TLC (SiO₂, DCM: MeOH=20: 1) to give tert-butyl (1R,5S)-3-(7-(1,5-dimethyl-6-(trifluoromethyl)-1H-indazol-7-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-251j_P1 & P2) and tert-butyl (1R,5S)-3-(7-(2,5-dimethyl-6-(trifluoromethyl)-2H-indazol-7-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-251k_P1 & P2) as yellow solid. MS (ESI): m/z (M+H)⁺ 746.

### Step M: 44(1R,5S)-3,8-diazabicyclo[3.2.1]octan-3-yl)-7-(1,5-dimethyl-6-(trifluoromethyl-1H-indazol-7-yl)-6,8-difluoro-2-(((2R,7aS)-2-fluorotetrahydro-1H-pyrrolizin-7a(5H)-yl)methoxylquinazoline (Ex. 251-A)

A solution of tert-butyl (1R,5S)-3-(7-(1,5-dimethyl-6-(trifluoromethyl)-1H-indazol-7-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-251j_P1) (12 mg, 0.016 mmol) in DCM (0.8 mL) and TFA (0.2 mL) was stirred at 20° C. for 1 h. LCMS showed the reaction was finished. The reaction was concentrated in vacuo and the residue was purified by Prep-HPLC (Column Waters Boston Prime C18 100*30 mm*5 um, Condition water (0.05% NH₃H₂O+10 mM NH₄HCO₃)-ACN, 100% B Hold Time (min) 2 to give 4-((1R,5S)-3,8-diazabicyclo[3.2.1]octan-3-yl)-7-(1,5-dimethyl-6-(trifluoromethyl)- 1H-indazol-7-yl)-6,8-difluoro-2-(((2R, 7aS)-2-fluorotetrahydro-1H-pyrrolizin-7a(5H)-yl)methoxy)quinazoline. MS (ESI): m/z (M+H)⁺ 646. Ex. 251-A_P: ¹H NMR (400 MHz, CDCl₃) δ 7.99 (s, 1H), 7.76 (s, 1H), 7.42 (d, J=9.52 Hz, 1H), 5.18-5.39 (m, 1H), 4.48 (br d, J=12.40 Hz, 1H), 4.22-4.30 (m, 2H), 4.13 (d, J=10.37 Hz, 1H), 3.68 (br s, 2H), 3.60 (br d, J=−12.16 Hz, 1H), 3.47 (br d, J=12.99 Hz, 1H), 3.45 (s, 3H), 3.21-3.32 (m, 2H), 3.18 (s, 1H), 2.89-3.07 (m, 1H), 2.66 (d, J=2.50 Hz, 3H), 2.11-2.35 (m, 3H), 1.85-2.00 (m, 7H). The procedures of Ex. 251-A_P2, Ex. 251-B_P1B, and Ex. 251-B_P2 were similar as Ex. 251-A_P1. Ex. 251-A_P2: ¹H NMR (400 MHz, CDCl₃) δ 7.99 (s, 1H), 7.76 (s, 1H), 7.43 (dd, J=1.43, 9.66 Hz, 1H), 5.20-5.37 (m,i 1H), 4.50 (br dJ=12.40 Hz, 1H), 4.22-4.30 (m, 2H), 4.14 (d, J=10.25 Hz, 1H), 3.69 (br s, 2H), 3.62 (br d, J=12.40 Hz, 1H), 3.45-3.50 (m, 1H), 3.44 (s, 3H), 3.22-3.33 (m, 2H), 3.18 (s, 1H), 2.94-3.03 (m, 1H), 2.66 (d, J=2.62 Hz, 3H), 2.29 (br d, J=4.41 Hz, 1H), 2.15-2.25 (m, 2H). 1.89-2.01 (m, 4H), 1.83-1.87 (m, 3H). MS (ESI): m/z (M+H)⁺ 646. Ex. 251_B_P1: ¹H NMR (400 MHz, CDCl₃) δ 7.89 (s, 1H), 7.66 (s, 1H), 7.41 (d, J=10.01 Hz, 1H), 5.20-5.38 (m, 1H), 4.38 (br d, J=12.87 Hz, 2H), 4.29 (d, J=10.49 Hz, 1H), 4.17 (br d, J=10.61 Hz, 1H), 4.13 (s, 3H), 3.69 (br s, 2H), 3.53 (br dd. J=3.93, 12.87 Hz, 2H), 3.23-3.36 (m, 2H), 3.21 (br s, 1H), 3.00 (br s, 1H), 2.63 (br s, 3H), 2.32 (br s, 1H), 2.18-2.26 (m, 2H), 1.86-1.97 (m, 7H). MS (ESI): m/z (M+H)⁺ 646. Ex. 251_B_P2: ¹H NMR (400 MHz, CDCl₃) δ 7.89 (s, 1H), 7.66 (s, 1H), 7.41 (dd, J=1.37, 9.83 Hz, 1H), 5.20-5.38 (m, 1H), 4.37 (br t, J=10.91 Hz, 2H), 4.23-4.31 (m, 1H), 4.16 (br d, J=10.25 Hz, 1H), 4.13 (s, 3H), 3.67 (br s, 2H), 3.51 (br dd, J=5.60, 12.16 Hz, 2H), 3.23-3.36 (m, 2H), 3.20 (s, 1H), 2.98 (br dd, J=3.76, 4.59 Hz, 1H), 2.63 (d, J=1.55 Hz, 3H), 2.31 (br s, 1H), 2.13-2.27 (m, 2H), 1.89-2.02 (m, 5H), 1.86 (br s, 2H). MS (ESI): m/z (M+H)⁺ 646.

### Ex 252: Synthesis of 1-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl))--ethynyl-7-fluoroisoquinolin-3-amine (Ex.252a and b)

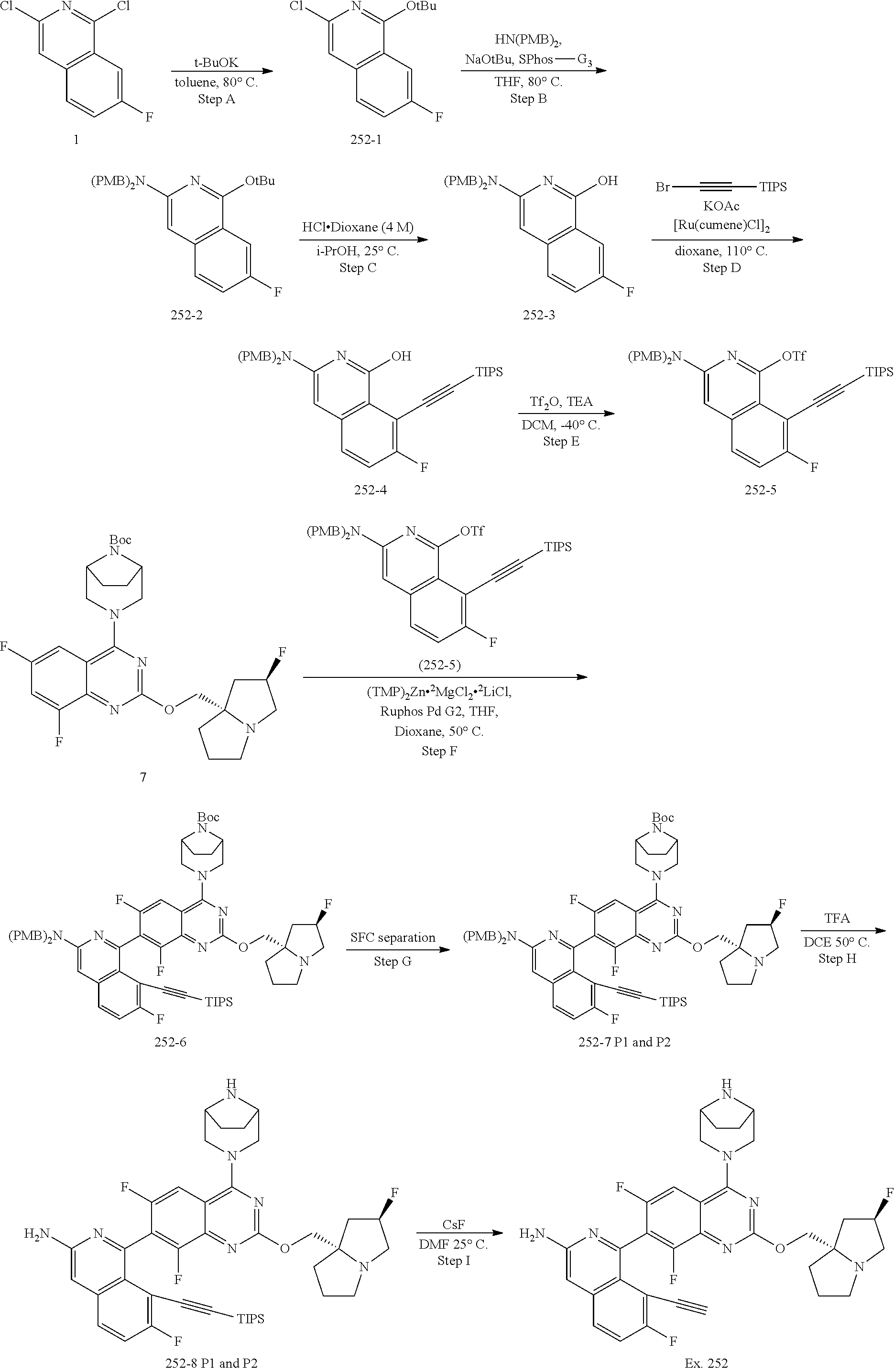

mL and H₂O filtered. The filtrate was concentrated under reduced pressure to give the residue which was purified by flash silica gel chromatography 40 g column, Eluent of 0 ˜ 1% EtOAc/Pet, ether gradient, dry loaded to give 1-(tert-butoxy)-3-chloro-7-fluoroisoquinoline. MS (ESI): m/z (M-t-Bu+H)⁺ 198. ¹H NMR (400 MHz, CDCl₃) δ 7.76 (dd, J=2.56, 9.48 Hz, 1H), 7.62 (dd, J=5.36, 8.94 Hz, 1H), 7.40 (dt, J=2.62, 8.64 Hz, 1H), 7.19 (s, 1H), 1.73 (s, 9H).

### Step B: 1-(tert-butoxy)-7-fluoro-N,N-bis(4-methoxybenzyl)isoquinolin-3-amine (Int-252-2)

A mixture of 1-(tert-butoxy)-3-chloro-7-fluoroisoquinoline (Int-252-1) (4.70 g, 18.5 mmol), bis(4-methoxybenzyl)amine (7.15 g, 27.8 mmol), sodium tert-butoxide (3.56 g, 37.1 mmol) and SPhos Pd G3 (1.45 g, 1.85 mmol) in THF (80 mL) was degassed and backfilled with N₂ for 3 times. The reaction mixture was stirred at 80° C. for 12 h under N₂ protection. TLC (SiO₂, Pet, ether/EtOAc=50:1, v/v) showed all the starting material was consumed completely. The reaction mixture was diluted with EtOAc (100 mL) and filtered. The filtrate was concentrated under reduced pressure to give the residue which was purified by flash silica gel chromatography 40 g column, Eluent of 0-5% EtOAc/Pet, ether gradient, dry loaded to give 1-(tert-butoxy)-7-fluoro-N,N-bis(4-methoxybenzyl)isoquinolin-3-amine. ¹H NMR (400 MHz, CDCl₃) δ 7.62 (dd, J=2.26, 9.78 Hz, 1H), 7.33 (dd, J=5.36, 8.82 Hz, 1H), 7.12-7.23 (m, 5H), 6.85 (d, J=8.58 Hz, 4H), 6.17 (s, 1H), 4.71 (s, 4H), 3.80 (s, 6H), 1.57-1.63 (m, 9H).

### Step C: 3-(bis(4-methoxybenzyl)amino)-7-fluoroisoquinolin-1-ol (Int-252-3)

To a solution of 1-(tert-butoxy)-7-fluoro-N,N-bis(4-methoxybenzyl)isoquinolin-3-amine (Int-252-2) (2.00 g, 4.21 mmol) in i-PrOH (10 mL) was added HCl/dioxane (10 mL). The reaction mixture was stirred at 25° C. for 4 h. The reaction mixture was concentrated under reduced pressure to give the residue which was diluted with EtOAc (20 mL) and NaHCO₃(15 mL). The aqueous layer was separated and extracted with EtOAc (20 mL×3). The combined organic layers were dried over Na₂SO₄ and filtered. The filtrate was concentrated under reduced pressure to give the residue which was purified by flash silica gel chromatography 20 g column, Eluent of 0 ˜ 36% EtOAc/Pet, ether gradient, dry loaded to give 3-(bis(4-methoxybenzyl)amino)-7-fluoroisoquinolin-1-ol. MS (ESI): m/z (M+H)⁺ 419. ¹H NMR (400 MHz, DMSO-d₆) δ 11.28 (s, 1H), 7.57-7.66 (m, 1H), 7.34-7.39 (m, 2H), 7.17 (d, J=8.70 Hz, 4H), 6.88 (d, J=8.58 Hz, 4H), 5.71 (s, 1H), 4.37 (s, 4H), 3.71 (s, 6H).

### Step D: 3-(bis(4-methoxybenzyl)amino)-7-fluoro-8-((triisopropylsilyl)ethynyl)isoquinolin-1-ol (Int-252-4)

A mixture of 3-(bis(4-methoxybenzyl)amino)-7-fluoroisoquinolin-1-ol (Int-252-3) (1.00 g, 2.39 mmol), (bromoethynyl)triisopropylsilane (0.999 g, 3.82 mmol), potassium acetate (0.469 g, 4.78 mmol) and dichloro(p-cymene)ruthenium(II) dimer (0.146 g, 0.239 mmol) in dioxane (15 mL) was degassed and backfilled with N₂ for 3 times. The reaction mixture was stirred at 110° C. for 15 h under N₂ protection. The reaction was filtered, and the filtrate was concentrated under reduced pressure to give the residue which was purified by flash silica gel chromatography 20 g column, Eluent of 0 ˜ 23% EtOAc/Pet, ether gradient, dry loaded to give 3-(bis(4-methoxybenzyl)amino)-7-fluoro-8-((triisopropylsilyl)ethynyl)isoquinolin-1-ol. MS (ESI): m/z (M+H)⁺ 599. ¹H NMR (400 MHz, CDCl₃) δ 7.18-7.23 (m, 1H), 7.12 (d,J=−8.58 Hz, 5H), 6.87 (d, J=7.38 Hz, 5H), 5.57 (s, 1H), 4.40 (s, 4H), 3.81 (s, 6H), 1.19 (s, 21H).

### Step E: 3-(bis(4-methoxybenzyl)amino)-7-fluoro-8-((triisopropylsilyl)ethynyl)isoquinolin-1-yl trifluoromethanesulfonate (Int-252-5)

To a solution of 3-(bis(4-methoxybenzyl)amino)-7-fluoro-8-((triisopropylsilyl)ethynyl)isoquinolin-1-ol (Int-252-4) (820 mg, 1.37 mmol) in DCM (15 mL) were added TEA (1.1 mL, 8.22 mmol) and Tf₂O (0.7 mL, 4.11 mmol) at −40° C. under N₂ protection. The reaction mixture was stirred at −40° C. for 20 min. TLC (SiO₂, Pet, ether !EtOAc=5:1, v/v) showed all the starting material was consumed completely. The reaction mixture was concentrated under reduced pressure to give the residue which was purified by flash silica gel chromatography 20 g column, Eluent of 0-32% EtOAc/Pet, ether gradient, dry loaded to give 3-(bis(4-methoxybenzyl)amino)-7-fluoro-8-((triisopropylsilyl)ethynyl)isoquinolin-1-yl trifluoromethanesulfonate. ¹H NMR (400 MHz, CDCl₃) δ 7.34-7.40 (m, 1H), 7.26-7.27 (m, 1H), 7.15 (d, J=8.58 Hz, 4H), 6.84-6.87 (m, 4H), 6.54 (s, 1H), 4.68 (s, 4H), 3.79-3.81 (m, 6H), 1.16-1.20 (m, 21H).

### Step F: Tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-252-6)

(TMP)₂Zn- MgCl₂—LiCl (4.9 mL, 1.97 mmol) (0.4 M in THF) was added to tert-butyl (1R,5S)-3-(6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (350 mg, 0.656 mmol) at 25° C. under N₂ atmosphere, and the mixture was stirred at 50° C. for 2 h. A solution of 3-(bis(4-methoxybenzyl)amino)-7-fluoro-8-((triisopropylsilyl)ethynyl)isoquinolin-1-yl trifluoromethanesulfonate (Int-252-5) (575 mg, 0.787 mmol) and RuPhos Pd G2 (50.9 mg, 0.066 mmol) in dioxane (5 mL) was added to the reaction solution at 25° C. in glove box, then the reaction mixture was stirred at 50° C. for 12 h. The reaction was monitored using LC. The reaction mixture was diluted with EtOAc (50 mL), quenched with NaHCO₃(10 mL) and the resulting mixture was filtered. The filtrate was washed with brine (20 mL). The organic layer was dried over anhydrous Na₂SO₄, filtered and concentrated in vacuo. The residue was purified by flash silica gel chromatography 20 g column, Eluent of 0-5% MeOH/DCM gradient, dry loaded to give tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI): m/z (M+H)⁺ III4.

### Step G: Tert-butyl (1R,5S)-3-(7-(3-(bis(4-methoxybenzyl)amino)-7-fluoro-8-((triisopropylsilyl)ethynylsoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3, 2.1]octane-8-carboxylate (Int-252-7 PI and Int-252-7 P2)

Tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.]octane-8-carboxylate (Int-252-6) (680 mg, 0.610 mmol) was separated by preparative SFC (Column REG1S (s,s) WHELK—O1 (250 mm*30 mm, 5 um), Condition 0.1% NH₃H₂O IPA, Begin B 50%, End B 50%, Gradient Time (min) 10, 100% B Hold Time 10, Flow Rate (mL/min) 80, Injections 300) to give tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-252-7_P1, the first eluting isomer from SFC) as a yellow solid and tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-252-7_P2, the second eluting isomer from SFC) as a yellow solid. 252-7_P1: MS (ESI): m/z (M/2+H)⁺ 558, 252-7_P2: MS (ESI): m/z (M/2+H)⁺ 558. Int-252-7_P1: ¹H NMR (400 MHz, CDCl₃) δ 7.47 (dd, J=5.42, 9.24 Hz, 1H), 7.32 (d.-9.78 Hz, 1H), 7.25 (d, 1J=8.94 Hz, 1H), 7.19 (d, J=8.58 Hz, 4H), 6.85 (d, J=8.70 Hz, 4H), 6.A (s, 1H), 5.36-5.21 (m, 1H), 4.67-4.78 (m, 4H), 4.32-4.59 (m, 3H), 4.26 (d, J=9.89 Hz, 1H), 4.08 (d, J=9.78 Hz, 1H), 3.94 (d, J=11.56 Hz, 1H), 3.80 (s, 6H), 3.59-3.75 (m, 1H), 3.11-3.38 (m, 4H), 2.97 (d, J=6.08 Hz, 1H), 2.13-2.35 (m, 3H), 1.86-2.10 (m, 6H), 1.65-1.74 (m, 1H), 1.52 (s, 9H), 0.87 (dd, J=7.51, 19.91 Hz, 18H), 0.57 (quin, J=7.39 Hz, 3H). Int-252-7_P2: ¹H NMR (400 MHz, CDCl₃) δ 7.47 (dd, J=5.48, 9.18 Hz, 1H), 7.32 (d, J=10.01 Hz, 1H), 7.22-7.26 (m, 1H), 7.19 (d, J=8.46 Hz, 4H), 6.85 (d, J=8.58 Hz, 4H), 6.64 (s, 1H), 5.36-5.20 (m, 1H), 4.73 (s, 4H), 4.51-4.61 (m, 1H), 4.36 (s, 2H), 4.24 (d, J=10.49 Hz, 1H), 4.11 (d, J=9.89 Hz, 1H), 3.92 (d, J=12.28 Hz, 1H), 3.80 (s, 6H), 3.61-3.75 (m, 1H), 3.12-3.36 (m, 4H), 2.98 (d, J=5.96 Hz, 1H), 2.06-2.32 (m, 4H), 1.83-2.03 (m, 5H), 1.69-1.76 (m, 1H), 1.52 (s, 9H), 0.83-0.92 (m, 18H). 0.56 (quin. J=7.42 Hz, 3H).

### Step H: 1-(4-((1R5S)-3,8-diazabicyclo[3.2.1]octan-3-yl)-6,8-difluoro-2-(((2R,7aS)-2-fluorotetrahydro-1H-pyrrolizin-7a(5H)-yl)methoxyl)quinazolin-7-yl)-7-fluoro-8-((triisoprov,lsilyl)ethynyl)isoquinolin-3-amine (Int-252-8 P1 and Int-252-8 P2)

To a solution of tert-butyl (1R,5S)-3-(7-(3-(bis(4-methoxybenzyl)amino)-7-fluoro-8-((triisopropylsilyl)ethynyl)isoquinolin-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (200 mg, 0.179 mmol) (Int-252-7_P1) in DCE (2 mL) was added TFA (2 mL). The reaction mixture was stirred at 50° C. for 1 h. The reaction mixture was concentrated in vacuum to give the residue which was diluted with DCM (10 mL), washed with NaHCO₃(aq.) (5 mL). The organic layer was dried over Na₂SO₄ and filtered. The filtrate was concentrated under reduced pressure to give 1-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-7-fluoro-8-((triisopropylsilyl)ethynyl)isoquinolin-3-amine (Int-252-8_P1) as black oil. The crude product was used in the next step without further purification. MS (ESI): m/z (M+H)⁺ 774.

### Step 1: 1-(4-((0R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-8-ethynyl-7-fluoroisoquinolin-3-anine (Ex.252 P1)

To a solution of 1-(4-((1R5S)-3,8-diazabicyclo[3.2.]octan-3-yl)-6,8-difluoro-2-(((2R,7aS)-2-fluorotetrahydro-1H-pyrrolizin-7a(5H)-yl)methoxy)quinazolin-7-yl)-7-fluoro-8-((triisopropylsilyl)ethynyl)isoquinolin-3-amine (129 mg, 0.167 mmol) (Int-252-8_P1) in DMF (2.5 mL) was added CsF (127 mg, 0.833 mmol). The reaction mixture was stirred at 25° C. for 1 h. The reaction mixture was quenched with H₂O (5 mL), and the aqueous layer was extracted with EtOAc (2 mL×3). The combined organic layers were dried over Na₂SO₄ and filtered. The filtrate was concentrated under reduced pressure to give the residue which was purified by Prep-HPLC (Column Boston Green ODS 150*30 mm*5 um, Condition water (0, 1% FA)—ACN, Begin B 5, End B 25, Gradient Time (min) 12, 100% B Hold Time 2 Flow Rate (mL/min) 25, Injections 2) to give 1-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-8-ethynyl-7-fluoroisoquinolin-3-amine as a yellow solid. MS (ESI): m/z (M+H)⁺ 618. Ex.252a_P1: ¹H NMR (500 MHz, MeOD) δ 7.76 (dd, 1-5.49, 9.31 Hz, 1H), 7.60 (d, J=9.46 Hz, 1H), 7.42 (t, J=9.00 Hz, 1H), 7.01 (s, 1H), 5.41-5.55 (M, 1H), 4.62 (br d, J=13.89 Hz, 1H), 4.51-4.57 (m, 3H), 4.11 (br s, 2H), 3.84 (br d, J=13.58 Hz, 1H), 3.61-3.79 (m, 4H), 3.48 (s, 1H), 3.32-3.35 (m, 1H), 3.32-3.35 (m, 1H), 2.51-2.63 (m, 1H), 2.30-2.37 (m, 1H), 2.20-2.26 (m, 2H), 2.04-2.14 (m, 5H). The procedure of below isomer was similar as Ex.252a_P1, spectrum data as below. MS (ESI): m/z (M+H)⁺ 618. Ex.252b_P2: ¹H NMR (500 MHz, MeOD) δ 7.75 (dd, J=5.49, 9.31 Hz. 1H), 7.61 (d, J=9.46 Hz, 1H), 7.42 (t, J=9.00 Hz, 1H), 7.01 (s, 1H), 5.42-5.60 (m, 1H), 4.52-4.67 (m, 4H), 4.17 (br d, J=9.77 Hz, 2H), 3.68-3.91 (m, 5H), 3.50 (s, 1H), 3.32-3.39 (m, 1H), 2.47-2.68 (m, 2H), 2.33-2.41 (m, 1H), 2.23-2.30 (m, 2H), 2.06-2.18 (m, 5H).

### Example 254: Synthesis of 4-(4-((1R,5S)-3,8-diazabicyclo[3.2.1]octan-3-yl6,8-difluoro-2-(((2R,7aS)-2-fluorotetrahydro-1H-pyrrolizin-7a(5H)-ylmethoxy)quinazolin-7-yl)-5-(difluoromethyl-6-methyl-1H-indazole-3-carbonitrile

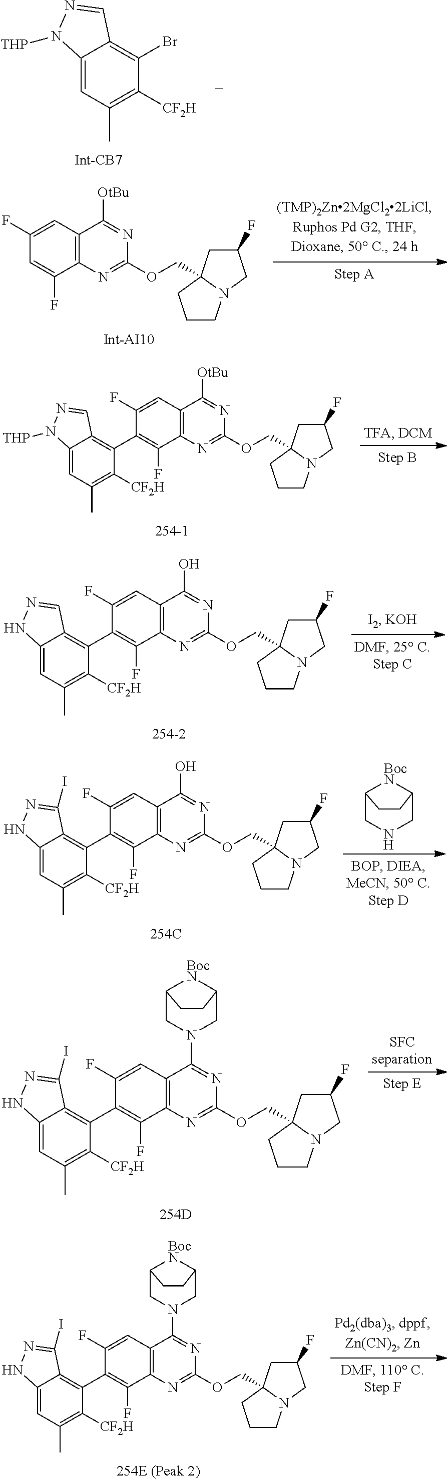

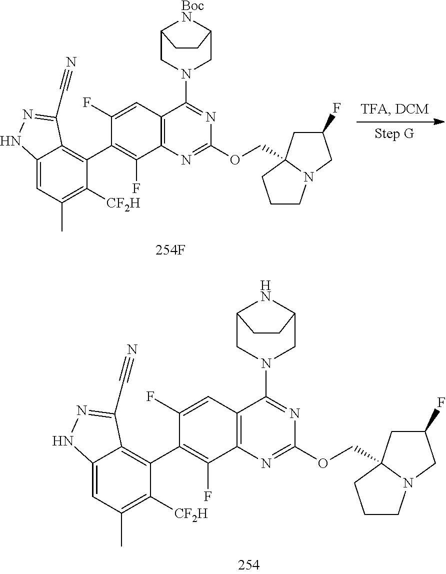 [SMILES: Cc1cc2[nH]nc(C#N)c2c(-c2c(F)cc3c(N4CC5CCC(C4)N5C(=O)OC(C)(C)C)nc(OC[C@@]45CCCN4C[C@H](F)C5)nc3c2F)c1C(F)F ; Cc1cc2[nH]nc(C#N)c2c(-c2c(F)cc3c(N4CC5CCC(C4)N5)nc(OC[C@@]45CCCN4C[C@H](F)C5)nc3c2F)c1C(F)F]

### Step A: 44tert-butoxy)-7-(5-(difluoromethyl)-6-methyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazoline (Int-254-1)

A mixture of 4-(tert-butoxy)-6,8-difluoro-2-(((2R,7aS)-2-fluorotetrahydro-1H-pyrrolizin-7a(5H)-yl)methoxy)quinazoline (1 g, 2.53 mmol) (Int-A110) in (TMP)₂Zn 2MgCl₂LiCl (18.97 mL, 7.59 mmol) (0.4 M in THF) was stirred at 50° C. for 1 h, and then a solution of RuPhos Pd G2 (0.196 g, 0.253 mmol) and 4-bromo-5-(difluoromethyl)-6-methyl-1-(tetrahydro-2H-pyran-2-yl)-1H-indazole (1.048 g, 3.03 mmol) (Int-CB7) in dioxane (14 mL) was added in glovebox. The mixture was stirred at 50° C. for 24 h. The mixture was diluted with EtOAc (100 mL), and quenched with sat. aq. NaHCO₃ (30 mL). The resulting mixture was filtered and the organic phase was concentrated in vacuo. The residue was purified by flash silica gel chromatography 20 g column, eluent of 0-40% EtOAc/Pet, ether gradient to give 4-(tert-butoxy)-7-(5-(difluoromethyl)-6-methyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazoline. MS (ESI) [M+H]⁺: m/z 660.

### Step B: 7-(5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-ol (Int-254-2)

To a solution of 4-(tert-butoxy)-7-(5-(difluoromethyl)-6-methyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazoline (700 mg, 1.061 mmol) (Int-254-1) in DCM (10 mL) was added TFA (3 mL, 38.9 mmol) at 25° C., and the reaction was stirred at 25° C. for 12 h. The reaction mixture was concentrated in vacuo, and the residue was diluted with DCM (5 mL). The mixture was basified with NaHCO₃aqueous solution (2 mL), and the mixture was extracted with DCM (5 mL×3). The combined organic phase was dried over anhydrous Na₂SO₄, filtered and concentrated in vacuo to give 7-(5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-ol. MS (ESI) [M+H]⁺: m/z 520.

### Step C: 7-(5-(difluoromethyl)-3-iodo-6-methyl-1H-indazol-4-yl)-6,8-difluoro-2-(((2R7aS)-2-fluorotetrahydro-1H-pyrrolizin-7a(5H)-yl)methoxyy)quinazolin-4-ol (Int-254C)

To a solution of 7-(5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-ol (551 mg, 1.061 mmol) (Int-254-2) in DMF (8 mL) was added KOH (119 mg, 2.121 mmol) and 12 (1077 mg, 4.24 mmol) at 25° C., and the reaction was stirred at 25° C. for 1 h. The reaction mixture was quenched with Na₂SO₃ aqueous solution (5 mL), and the mixture was extracted with DCM/MeOH (9:1, 30 mL ×3). The combined organic phase was dried over anhydrous Na₂SO₄, filtered and concentrated in vacuo. The residue was purified by flash silica gel chromatography 12 g column, DCM/MeOH=10/1) to give 7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-ol. MS (ESI) [M+H]⁺: m % z 646

### Step D: Tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-254D)

To a solution of 7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-ol (780 mg, 1.209 mmol) (Int-254C) in MeCN (15 mL) was added BOP (802 mg, 1.813 mmol) at 25° C., and the mixture was stirred at 25° C. for 30 min. DIEA (781 mg, 6.04 mmol) and tert-butyl (1R,5S)-3,8-diazabicyclo[3.2.1]octane-8-carboxylate (513 mg, 2.417 mmol) was added to the reaction solution, and then the mixture was stirred at 50° C. for 2 h. The reaction mixture was concentrated in vacuo, and the residue was purified by preparative TLC plate (SiO₂, DCM/MeOH (7M NH₃/MeOH)=10/1), followed by preparative HPLC (Column: Boston Green ODS 150×30 mm×5 um; Condition: water (0.1% TFA)-ACN Begin B 45, End B 65 Gradient Time (min) 20, 100% B Hold Time (min) 2 FlowRate (mL/min) 14) to give tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI) [M+H]⁺: m/z 840.

### Step E: Tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-254E)

The mixture of tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (3M) mg, 0.357 mmol) (Int-254D) was separated by preparative SFC (Column DAICEL CHIRALCEL OD-H (250 mm×30 mm, 5 um); Condition 0.1% NH₃H₂O EtOH. Begin B 50%, End B 50%: Gradient Time 10 (min) 100% B Hold Time 10 (min); FlowRate (mL/min) 75) to afford tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate, the second eluting isomer from SFC. MS (ESI) [M+H]⁺: m/z 840.

### Step F: tert-butyl (1R,5S)-3-(7-(3-cyano-5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-254F)

To a mixture of tert-butyl (1R,5S)-3-(7-(5-(difluoromethyl)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (90 mg, 0.107 mmol) (Int-254E) in DMF (1.5 mL) was added zinc (14.02 mg, 0.214 mmol), zinc cyanide (37.8 mg, 0.322 mmol), dppf (11.88 mg, 0.021 mmol) and tris(dibenzyldeneacetone)dipalladium(0) (9.82 mg, 10.72 μmol) at 25° C., and the reaction was stirred at 110° C. for 15 min under nitrogen atmosphere. The reaction mixture was diluted with EtOAc (20 mL), and then filtered. The organic phase was washed with H₂O (3 mL×3) and brine (3 mL×3), and dried over anhydrous Na₂SO₄, filtered and concentrated in vacuo. The residue was purified by preparative TLC plate (SiO₂. DCM/MeOH=10/1) to give tert-butyl (1R,5S)-3-(7-(3-cyano-5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI) [M+H]+: m/z 739

### Step G: 4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-(difluoromethyl)-6-methyl-1H-indazole-3-carbonitrile (Ex.254)

To a mixture of tert-butyl (1R,5S)-3-(7-(3-cyano-5-(difluoromethyl)-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (65 mg, 0.088 mmol) (Int-254F) in DCM (1.5 mL) was added TFA (0.5 mL, 6.49 mmol) at 25° C., and the reaction was stirred at 25° C. for 1 h. LCMS showed the starting material was consumed and desired MS was formed. The reaction mixture was concentrated in vacuo, and the residue was diluted with MeOH (2 mL). The mixture was basified with NH₃H₂O, and the mixture was purified by preparative HPLC (Column: Boston Prime C18 150×30 mm×5 um; Condition: water (0.05% NH₃H₂0+10 mM NH₄HCO₃)-ACN Begin B 35, End B 65 Gradient Time (min) 10, 100% B Hold Time (min) 2 FlowRate (mL/min) 25) to give 4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-(difluoromethyl)-6-methyl-1H-indazole-3-carbonitrile. MS (ESI) [M+H]⁺: m/z 639. ¹H NMR (400 MHz, MeOD) δ7.78 (s, 1H), 7.69 (d, J=10.0 Hz, 1H), 6.82 (t, J=53.2 Hz, 1H), 5.32 (d, J=54.8 Hz, 1H), 4.59-4.42 (m, 2H), 4.32 (d, J=10.8 Hz, 1H), 4.24 (d, J=10.8 Hz, 1H), 3.72-3.54 (m, 4H), 3.32-3.16 (m, 3H), 3.10-2.97 (m, 1H), 2.77 (s, 3H), 2.44-2.12 (m, 3H), 2.07-1.97 (m, 2H), 1.96-1.77 (m, 5H).

### Example 255: Synthesis of 4-(4-((1R,5S)-3,8-diazabicyclo[3.2.1]octan-3-yl)-68-difluoro-2-(((2R,7aS)-2-fluorotetrahydro-1H-pyrrolizin-7a(5H)-yl)methoxy)quinazolin-7-yl)-5-(chlorodifluoromethoxy)-6-methyl-1H-indazole-3-carbonitrile

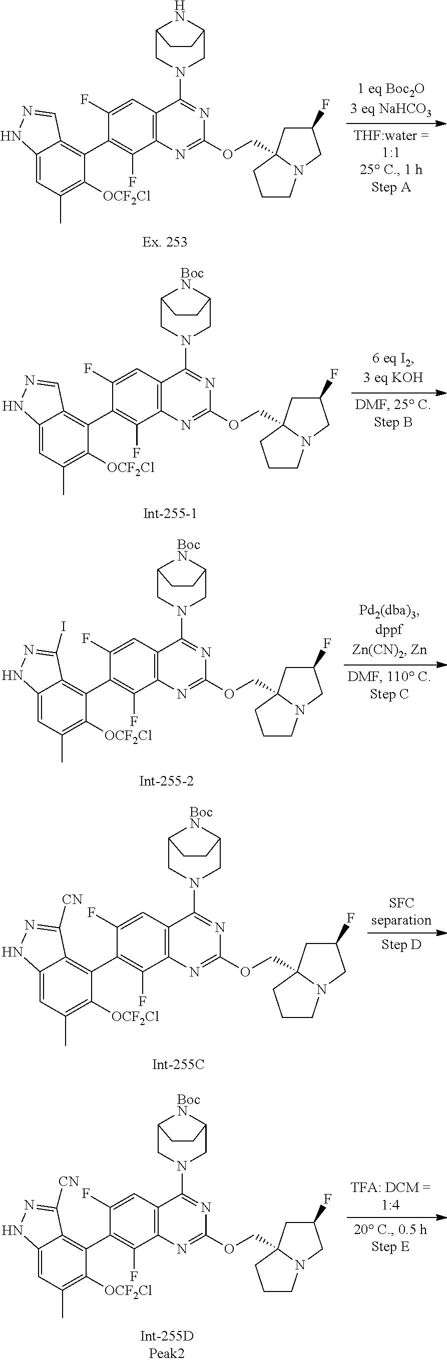

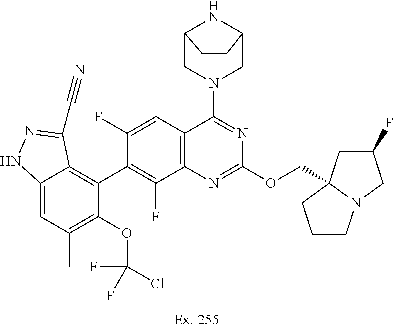 [SMILES: Cc1cc2[nH]nc(C#N)c2c(-c2c(F)cc3c(N4CC5CCC(C4)N5)nc(OC[C@@]45CCCN4C[C@H](F)C5)nc3c2F)c1OC(F)(F)Cl]

### Step A: Tert-butyl (1R,5S)-3-(7-(5-(chlorodifluoromethoxy)-6-methyl-1H-indazol-4-Y1)-6,8-difluoro-2-(((2R7aS)-2-fluorotetrahydro-1H-pyrrolizin-7a(5H)-yl)methoxy)quinazolin-4-yl)-3,8-diazabicyclo[3.2.1]octane-8-carboxylate (Int-255-1)

To a stirred solution of 4-((1R5S)-3,8-diazabicyclo[3.2.1]octan-3-yl)-7-(5-(chlorodifluoromethoxy)-6-methyl-1H-indazol-4-yl)-6,8-difluoro-2-(((2R,7aS)-2-fluorotetrahydro-1H-pyrrolizin-7a(5H)-yl)methoxy)quinazoline (390 mg, 0.587 mmol) (Ex.253) in THF (2 mL) was added sodium bicarbonate (148 mg, 1.762 mmol), H₂O (2 mL) and di-tert-butyl dicarbonate (0.135 mL, 0.587 mmol) at 25° C. The mixture was stirred at 25° C. for 1 h and monitored by LCMS, The mixture was diluted with water (3 mL) and extracted with EtOAc (3×10 mL). The organic laver was dried over sodium sulfate, filtered and the solvent was evaporated under reduced pressure. The crude product was purified by preparative Prep-TLC (DCM/MeOH=10:1) to give tert-butyl (1R,5S)-3-(7-(5-(chlorodifluoromethoxy)-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI) [M+H]⁺: m- 764.

### Step B: Tert-butyl (1R,5S)-3-(7-(5-(chlorodifluoromethoxy)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-255-2)

To a solution of tert-butyl (1R,5S)-3-(7-(5-(chlorodifluoromethoxy)-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (260 mg, 0.340 mmol) (Int-255-1) in DMF (3 ml) was added KOH (57.3 mg, 1.021 mmol) and I₂ (345 mg, 1,361 mmol) at 25° C., and the reaction was stirred at 25° C. for 1 h. The mixture was diluted with water (5 mL) and extracted with EtOAc (3×10 mL). The organic layer was washed with brine (2×5 mL), dried over sodium sulfate, filtered and the solvent was evaporated under reduced pressure. The residue was purified by preparative TLC (SiO₂, DCM: MeOH=15: 1) to give tert-butyl (1R,5S)-3-(7-(5-(chlorodifluoromethoxy)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI) [M+H]⁺: m/z 890.

### Step C: Tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-255C)

To a mixture of tert-butyl (1R,5S)-3-(7-(5-(chlorodifluoromethoxy)-3-iodo-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (257 mg. 0.289 mmol) (Int-255-2) in DMF (2.5 ml) was added zinc (37.8 mg, 0.577 mmol), zinc cyanide (190 mg, 1.618 mmol), dppf (32.0 mg, 0.058 mmol) and tris(dibenzyldeneacetone)dipalladium(0) (26.4 mg, 0.029 mmol). The reaction was stirred at 110° C. for 15 min under nitrogen. The reaction mixture was diluted with EtOAc (10 mL), and then filtered. The filtrate was washed with H₂O (3 ml×2) and brine (3 ml×2). The organic phase was dried over anhydrous Na₂SO₄, filtered and concentrated in vacuo. The residue was purified by preparative TLC (S102, DCM:MeOH=10:1) to give tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. MS (ESI) [M+H]⁺: m/z 789

### Step D: Tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (Int-255D)

The racemic tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (180 mg, 0.228 mmol) (Int-255C) was separated by preparative SFC (Column REG1S (s,s) WHELK-O1 (250 mm×30 mm, 5 um), Condition 0.1% NH₃H₂O IPA Begin B 40, End B 40 Gradient Time (min) 10, 100% B Hold Time (min) 10, FlowRate (mL/min) 80, Injections 60) to give tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate, the second eluting isomer from SFC.

### Step E: 4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-(chlorodifluoromethoxy)-6-methyl-1H-indazole-3-carbonitrile (Ex.255)

To a solution of tert-butyl (1R,5S)-3-(7-(5-(chlorodifluoromethoxy)-3-cyano-6-methyl-1H-indazol-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (78 mg, 0.099 mmol) (Int-255D) in TFA:DCM=1:4 (1 ml) was stirred at 20° C. for 0.5 h. The mixture was blew dry with nitrogen to remove most TFA and DCM. Then the mixture was diluted with aq. NaHCO₃(2 mL) and extracted with DCM (3×5 mL). The organic layer was dried over sodium sulfate, filtered and the solvent was evaporated under reduced pressure to give the crude product. The residue was purified by reverse preparative HPLC (Column: Boston Prime C18 150×30 mm×5 um; Condition: Water (0.05% NH₃H₂O+10 mM NH₄HCO₃)-ACN; Begin B 35 End B: 65; Gradient Time (min): 10; 100% B Hold Time (min): 2; FlowRate (mL/min): 25) to give 4-(4-((1R,5S)-3,8-diazabicyclo[3.2.1]octan-3-yl)-6,8-difluoro-2-(((2R,7aS)-2-fluorotetrahydro- H-pyrrolizin-7a(5H)-yl)methoxy)quinazolin-7-yl)-5-(chlorodifluoromethoxy)-6-methyl-1H-indazole-3-carbonitrile (Ex.255). MS (ESI) [M+H]⁺: m/z 689.3, ¹H NMR (400 MHz, MeOD) S: 7.73 (s, 1H), 7.58 (dd, J=1.6, 10.4 Hz, 1H), 5.22 (d, J=54.0 Hz, 1H), 4.42 (d, J=12.4 Hz, 1H), 4.29 (d, J=11.6 Hz, 1H), 4.22 (d, J=10.4 Hz, 1H), 4.14 (d, J=10.4 Hz, 1H), 3.55-3.58 (m, 3H), 3.50 (d, J=12.8 Hz, 1H), 3.12-3.17 (m, 3H), 2.91-2.95 (m, 1H), 2.48 (s, 3H), 2.09-2.19 (m, 3H), 1.89-1.93 (m, 2H), 1.74-1.78 (m, 5H).

### Assay Descriptions

### Procedure for SOS-Catalyzed Nucleotide Exchange Assay

The SOS-catalyzed nucleotide exchange assay utilizes a preformed complex of recombinant biotinylated KRAS protein containing G12D mutation (SEQ ID NO: 1), Bodipy-GDP, and Terbium-streptavidin. Compounds are added to this complex and then after a 60-minute incubation time the mixture is treated with SOS and unlabeled GTP. Small molecule inhibitors stabilize the Bodipy-GDP complex whereas the untreated protein rapidly exchanges Bodipy-GDP for unlabeled GTP resulting in reduced TR-FRET signal.

Biotinylated KRAS G12D protein is diluted to 2 μM in an EDTA buffer (20 mM HEPES pH 7.5, 50 mM sodium chloride, 10 mM EDTA, and 0.01% Tween 20) and incubated at room temperature for one hour. This mixture is then further diluted to 90 nM in an assay buffer (20 mM HEPES pH 7.5, 150 mM sodium chloride, 10 mM magnesium chloride, and 0.005% Tween) containing 15 nM of Terbium-Streptavidin (Invitrogen, catalog #PV3577) and 900 nM of Bodipy-GDP (Invitrogen, G22360) and incubated at room temperature for six hours. This solution is referred to as biotinylated KRAS G12D stock solution. For use in the final assay, the biotinylated stock solution is diluted to 1.5 nM KRAS G12D in assay buffer to generate the biotinylated KRAS G12D assay solution.

Each test compound (10 mM stock in DMSO) is diluted in DMSO to make a 10-point, 3-fold dilution series in a 384-well low dead volume microplate (Labcyte, catalog #LP-0200). Once titrations are made, 10 nL of the diluted compounds is acoustically dispensed into 384-well plates (Corming, catalog #3820) using an Echo 550 (Labcyte).

Each well of the assay plate receives 6 μL of Biotinylated KRAS G12D assay mixture and is incubated at room temperature for 60 minutes. Each well then receives 3 μL of 120 nM recombinant human SOS protein and 9 mM GTP (Sigma, G8877) in assay buffer and is incubated at room temperature for 60 minutes.

The time-resolved fluorescence resonance energy transfer signal is measured on an Envision (PerkinElmer) plate reader: Excitation filter=340 nm; emissionI=495 nm; emission2=520 nm; dichroic mirror=D400/D505; delay time=100 ms. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation.

### Procedure for RAS:RAF-RBD Binding Assay

The RAF-Ras binding domain (RBD) protein interaction assay utilizes recombinant biotinylated KRAS protein containing a G12D mutation (SEQ ID NO: 1) and the GST-tagged Ras binding domain of C-RAF (residues 50-132) from Jena Biosciences (catalog #PR-366). Compounds are added to KRAS and then after a 30-minute incubation time the RAF-RBD and detection antibodies are added. Small molecule inhibitors that block the interaction of c-RAF-RBD prevent generation of a TR-FRET signal.

Biotinylated KRAS G12D protein is diluted to 20 nM in assay buffer (20 mM HEPES pH 7.5, 150 mM sodium chloride, 10 mM magnesium chloride, and 0.01% Tween20).

Each test compound (10 mM stock in DMSO) is diluted in DMSO to make a 10-point, 3-fold dilution series in a 384-well low dead volume microplate (Labcyte, catalog #LP-0200). Once titrations are made, 50 nL of the diluted compounds is acoustically dispensed into 384-well plates (Corning, catalog #3820) using an Echo 655 (Labcyte).

Each well of the assay plate receives 5 μL of Biotinylated KRAS G12D assay solution and is incubated at room temperature for 30 minutes. Each well then receives 5 μL of 100 nM GST-C-RAF RBD protein and a 1:100 dilution of both anti-GST-d₂ (Cisbio catalog #61GSTDLA) and Streptavidin-Tb cryptate (Cisbio catalog #610SATLA) in assay buffer and the plate is mixed and briefly centrifuged followed by a 60 minute incubation at room temperature.

The time-resolved fluorescence resonance energy transfer signal is measured on an Envision (PerkinElmer) plate reader: dichroic mirror=LANCE/DELFIA DUAL/Bias; Emissionl=615 nm; Emission2=665 nm; delay time=60 ms. The signal of each well is determined as the ratio of the emission at 665 nm to that at 615 nm. Percent effect of each well is determined after normalization to control wells containing DMSO (no effect) or a saturating concentration of inhibitor (max effect). The apparent effect as a function of compound concentration is fit to a four-parameter logistic equation.

### Procedure for Cellular Phospho-ERK Assay

AsPC-1 cells (ATCC) CRL-1682™), containing homozygous KRAS-G12D activating mutation, were cultured in T150 flask in growth medium (RPMI medium 1640-GlutaMAXT-I (ThermoFisher Scientific 61870). The cells were harvested in growth medium after TrypLE (ThermoFisher scientific 12604021) digestion and were seeded in a 384-well collagen coated cell culture plate (Corning 356702) at a density of 15,000 cells/well, and incubated at 37° C. 5% CO? overnight. The compound dose-response titrations were prepared and appropriate amounts of compounds (Examples 1a-106) were dispensed in a 384-well intermediate plate using an Echo 550liquid handler. RPMI medium 1640-GlutaMAX™-I was added to the intermediate plate and transferred to a 384-well cell culture plate, which was incubated at 37° C., 5% CO₂ for 2 hours. After removal of medium from the plate, cells were lysed in lysis buffer from Alpha SureFire® Ultra™ Multiplex p-Erk and total Erk assay kit (PerkinElmer MPSU-PTERK) containing Halt™ Protease and Phosphotase inhibitor cocktail (ThermoFisher Scientific 78446) at room temperature with constant shaking at 300 rpm for 30 minutes. The cell lysates were then transferred to OptiPlate-384 μlate (PerkinElmer 6005620) and the phosphorylation of Erk and total Erk levels were detected by Alpha SureFire Ultra Multiplex p-Erk kit (PerkinElmer MPSU-PTERK) following the manufacturer's protocol. Assay plates were read on a EnVision Multimode Plate Reader (PerkinElmer) and the ratio of p-Erk vs total Erk in each well was used as the final readout. Dose response curves were analyzed using a 4-parameter logistic model to calculate ICso values for compound Examples 1a-106 using spotfire software.

### Procedure for Cellular Phospho-ERK Assay

AsPC-1 cells (ATCC) CRL-1682™), containing homozygous KRAS-G12D activating mutation, were cultured in T150 flask in growth medium (RPMI medium 1640-GlutaMAX™-I (ThermoFisher Scientific 61870) containing 10% fetal bovine serum (ThermoFisher Scientific 10091148)). The cells were harvested in growth medium after TrypLE (ThermoFisher scientific 12604021) digestion or assay ready frozen cells after wash in growth medium and were seeded in a 384-well collagen coated cell culture plate (Corning 356702) at a density of 15,000 cells/well, and incubated at 37° C., 5% CO₂ overnight. The compound dose-response titrations were prepared and appropriate amounts of compounds (Examples 107a-252b) were dispensed in a 384-well intermediate plate using an Echo 550 liquid handler. RPMI medium 1640-GlutaMAX™-I was added to the intermediate plate and transferred to a 384-well cell culture plate, which was incubated at 37° C. 5% CO₂ for 2 hours. After removal of medium from the plate, cells were lysed in lysis buffer from Alpha SureFire® Ultra™ Multiplex p-Erk and total Erk assay kit (PerkinElmer MPSU-PTERK) containing Halt™ Protease and Phosphotase inhibitor cocktail (ThermoFisher Scientific 78446) at room temperature with constant shaking at 300 rpm for 30 minutes. The cell lysates were then transferred to OptiPlate-384 μlate (PerkinElmer 6005620) and the phosphorylation of Erk and total Erk levels were detected by Alpha SureFire Ultra Multiplex p-Erk kit (PerkinElmer MPSU-PTERK) following the manufacturer's protocol. Assay plates were read on a EnVision Multimode Plate Reader (PerkinElmer) and the ratio of p-Erk vs total Erk in each well was used as the final readout. Dose response curves were analyzed using a 4-parameter logistic model to calculate ICso values for compound Examples 107a-256 using spotfire software.

Table of Activities

| | IC₅₀ (nM) | | |
|---|---|---|---|
| | pERK | IC₅₀ (nM) | IC₅₀ (nM) |
| Example | (Cell) | (GNE) | (cRaf) |
| 1a | 14650 | 741.5 | |
| 2 | 33250 | 660 | |
| 3a | 3608 | 505.9 | |
| 3b | 8243 | 2156 | |
| 4 | 50850 | 169.3 | |
| 5 | 6638 | 106.5 | 17330 |
| 6a | 13320 | 159.2 | |
| 6b | 5500 | 426.3 | |
| 7 | 5025 | 35.53 | 2990 |
| 8a | 22930 | 7003 | |
| 8b | 12340 | 2182 | |
| 9 | 13630 | 1331 | |
| 10 | 9832 | 2748 | |
| 11a | 2076 | 24.08 | 4244 |
| 11b | 6258 | 131.1 | 7235 |
| 12a | 396.1 | 1.892 | |
| 12b | 840.8 | 2.074 | |
| 13 | 50850 | 5879 | |
| 14 | 23980 | 1572 | |
| 15 | 50850 | 2143 | |
| 16 | 5765 | 30.15 | 871.4 |
| 17 | 7430 | 52.08 | 3263 |
| 18a | 20580 | 8.971 | 292.9 |
| 18b | 50850 | 77.25 | 2678 |
| 19a | 50850 | 1177 | |
| 19b | 50850 | 1791 | |
| 20 | 3504 | 116.9 | 14380 |
| 21 | 635.9 | 14.44 | 1984 |
| 22a | 50850 | 5085 | |
| 22b | 38320 | 78.93 | 8450 |
| 23 | 44780 | 4277 | |
| 24 | 7109 | 279.5 | 10800 |
| 25 | 7068 | 454.2 | 20270 |
| 26 | 10430 | 358.5 | 11790 |
| 27 | 22540 | 496 | |
| 28 | 50850 | 7448 | |
| 29 | 11600 | 705.2 | |
| 30 | 30080 | 7613 | |
| 31 | 2536 | 116.8 | 1793 |
| 32 | 2256 | 64.23 | 1426 |
| 33 | 3449 | 88.54 | 5073 |
| 34 | 11530 | 2559 | 38280 |
| 35 | 2258 | 27.3 | 1893 |
| 36 | 46380 | 4679 | 50000 |
| 37 | 16710 | 571 | |
| 38a | 1277 | 45.24 | 616.9 |
| 38b | 22160 | 3400 | |
| 39 | 3307 | 46.73 | 4673 |
| 40 | 21960 | 689 | |
| 41 | 2842 | 20.28 | 1273 |
| 42 | 8456 | 17.46 | 20450 |
| 43 | 12550 | 74.31 | 2196 |
| 44 | 13860 | 1165 | |
| 45 | 5473 | 376.4 | 31640 |
| 46 | 50850 | 9342 | |
| 47 | 5787 | 551.1 | |
| 48 | 6125 | 528.7 | |
| 49 | 4991 | 183.9 | 24740 |
| 50 | 20300 | 5184 | |
| 51 | 11170 | 3934 | |
| 52 | 2563 | 132.2 | 3825 |
| 53 | 11010 | 2785 | |
| 54 | 14060 | 2647 | |
| 55 | 7065 | 968.7 | |
| 56 | 6917 | 4030 | |
| 57 | 6850 | 403.5 | 31530 |
| 58 | 5905 | 255.5 | 45320 |
| 59 | 2212 | 20.13 | |
| 60 | 8759 | 1589 | |
| 61 | 7577 | 38.74 | 3350 |
| 62 | 1860 | 3.832 | 334.1 |
| 63 | 26770 | 1592 | |
| 64 | 4385 | 146.4 | |
| 65 | 2841 | 272.4 | 45680 |
| 66a | 3459 | 54 | |
| 66b | 2115 | 31.1 | |
| 67 | 2860 | 173.3 | 4037 |
| 68 | 2377 | 39.89 | 3298 |
| 69 | 275.2 | 1.761 | 194 |
| 70 | 2334 | 43.5 | 5386 |
| 71a | 504.7 | 18.92 | |
| 71b | 4241 | 1340 | |
| 72 | 2498 | 30.76 | 7443 |
| 73 | 14020 | 1249 | |
| 74 | 2003 | 69.15 | |
| 75 | 7159 | 1438 | |
| 76a | 592.9 | 12.03 | 1813 |
| 76b | 4530 | 596.9 | |
| 77 | 5349 | 791.8 | |
| 78 | 6367 | 1633 | |
| 79 | 1929 | 72.47 | 4413 |
| 80a | 737.4 | 31.83 | 4051 |
| 80b | 5969 | 1316 | |
| 81 | 466.6 | 16.51 | 1978 |
| 82 | 2195 | 89.32 | 14750 |
| 83 | 640.2 | 2.721 | |
| 84 | 6157 | 155.6 | 9324 |
| 85 | 5621 | 255 | 18900 |
| 86 | 213.2 | 0.7234 | 267.9 |
| 87 | 1336 | 37.4 | 11380 |
| 88 | 13230 | 1350 | |
| 89 | 45.99 | 0.5645 | 31.84 |
| 90 | 564.1 | 0.5645 | |
| 91a | 17630 | 11110 | |
| 91b | 4165 | 1023 | |
| 92a | 13150 | 11110 | |
| 92b | 5315 | 235.7 | 9211 |
| 93 | 7127 | 436.7 | 35340 |
| 94 | 8473 | 116.1 | 32920 |
| 95 | 10770 | 73.39 | 15860 |
| 96a | 20810 | 1280 | |
| 96b | 657.2 | 3.315 | 637.1 |
| 97 | 24340 | 3948 | |
| 98a | 7267 | 380.2 | 34160 |
| 98b | 50850 | 11110 | |
| 99 | 242.7 | 2.458 | 57.48 |
| 100 | 99.79 | 1.56 | 89.94 |
| 101 | 42190 | 1022 | 20930 |
| 102 | 9.488 | 0.6391 | 14.06 |
| 103a | 3.76 | 1.054 | 26.22 |
| 103b | 2478 | 457.8 | |
| 104 | 11.8 | 1.054 | 5.933 |
| 105 | 93.08 | 6.466 | 1030 |
| 106 | 76.11 | 2.087 | 49.42 |

| | IC50 (nM) | |
|---|---|---|
| | pERK (cell | IC50 |
| | with FBS | (nM) |
| Example # | 10%) | GNE |
| 107a | 30000 | 648 |
| 107 | 30000 | 25.36 |
| 108 | | 3284 |
| 109 | | 2680 |
| 110 | | 770.3 |
| 111 | | 2471 |
| 112 | | 2727 |
| 113 | | 696.7 |
| 114 | | 793.8 |
| 115 | 1468 | 10.46 |
| 116 a | | 2894 |
| 116 b | 14020 | 473.4 |
| 117 | 2744 | 237.2 |
| 118 | 1807 | 82.63 |
| 119 | 30000 | 1442 |
| 120 | | 1437 |
| 121 | 11390 | 615.1 |
| 122 | 30000 | 964.4 |
| 124 | | 1.214 |
| 125 | | 148.5 |
| 126a | 1241 | 8.591 |
| 126b | 3463 | 67.92 |
| 127 | 3596 | 39.04 |
| 128a | 43.38 | 1.279 |
| 128b | 2847 | 232.1 |
| 129a | 2813 | 17.71 |
| 129b | 3006 | 14.11 |
| 130 | | 4193 |
| 131 | 6985 | 188.4 |
| 132 | 5581 | 346.6 |
| 133a | 3470 | 61.42 |
| 133b | 409.8 | 1.162 |
| 134 | 13630 | 870.4 |
| 135 | 469.2 | 54.61 |
| 136a | 644.7 | 4.935 |
| 136b | 2380 | 32.37 |
| 137a | | 1614 |
| 137b | | 4819 |
| 138 | | 360.8 |
| 139a | | 1670 |
| 139b | | 287.3 |
| 140 | 112.8 | 3.88 |
| 141 | 16410 | 138.6 |
| 142 | 383.8 | 1.575 |
| 143a | 53.51 | 0.86 |
| 143b | 2497 | 74.47 |
| 144a | 83.94 | 2.029 |
| 144b | 391.6 | 20.04 |
| 145a | 19440 | 1270 |
| 145b | 358.5 | 14.13 |
| 146a | 185.3 | 1.56 |
| 146b | | 25.25 |
| 147a | 16570 | 399.8 |
| 147b | 10200 | 429.3 |
| 148a | 234.8 | 13.64 |
| 148b | 8553 | 750.9 |
| 149 | 4491 | 103.1 |
| 150 | | 2.035 |
| 151a | 3952 | 1934 |
| 151b | 277.5 | 21.56 |
| 152 | 5194 | 848.1 |
| 154a | 52.1 | 1.098 |
| 154b | | 381.1 |
| 155a | | 330.1 |
| 155b | 144.4 | 2.33 |
| 156a | | 13.98 |
| 156b | | 553.8 |
| 157a | | 112.8 |
| 157b | | 2938 |
| 158 | | 1278 |
| 159 | 138.6 | 4.28 |
| 160a | 881.5 | 3.30 |
| 160b | 12470 | 367.4 |
| 161 | | 590.6 |
| 162a | 658.7 | 23.05 |
| 162b | 2390 | 495.5 |
| 163a | 2268 | 47.54 |
| 163b | 35590 | 4744 |
| 164 | 29330 | 562.4 |
| 165 | 15420 | 148.8 |
| 166a | 30000 | 944.5 |
| 166b | 152 | 4.40 |
| 167 | 8441 | 414.1 |
| 168 | | 1312 |
| 169 | 72.99 | 0.70 |
| 170a | 1382 | 4.72 |
| 170b | 14900 | 874.2 |
| 171a | 7717 | 1264 |
| 171b | | 3470 |
| 172 | 3499 | 17.08 |
| 173-1a | 1572 | 8.425 |
| 173-1b | 2888 | 22.06 |
| 173-2a | 10200 | 266 |
| 173-2b | | 2229 |
| 174a | | 3128 |
| 174b | | 1002 |
| 175a | 6450 | 738.3 |
| 175b | | 11110 |
| 176a | 399.8 | 1.912 |
| 176b | 4361 | 109.8 |
| 177a | 12.65 | 0.80 |
| 177b | 588.5 | 51.66 |
| 178a | 107.9 | 1.73 |
| 178b | 4576 | 210.1 |
| 179a | | 5.07 |
| 179b | | 727 |
| 180a | 1035 | 6.48 |
| 180b | 14240 | 744 |
| 181a | 122 | 3.97 |
| 181b | 2663 | 141.2 |
| 182 | 7904 | 455.9 |
| 183a | 6070 | 346.8 |
| 183b | 190 | 3.95 |
| 184 | | 4715 |
| 185 | 7037 | 732.1 |
| 186a | 179.7 | 1.49 |
| 186b | 3785 | 184.6 |
| 187 | 1096 | 27.32 |
| 188 | 30000 | 3402 |
| 189a | 671.6 | 12.75 |
| 189b | | 11110 |
| 190a | 30000 | 2506 |
| 190b | 5827 | 71.97 |
| 191a | 287.9 | 26.53 |
| 191b | 30000 | 2840 |
| 192a | 113.8 | 1.173 |
| 192b | 1569 | 36.97 |
| 193 | 186.8 | 10.34 |
| 194 | 11830 | 1921 |
| 195 | 79.62 | 4.355 |
| 196 | 3413 | 45.68 |
| 197 | 4472 | 52.44 |
| 198 | | 1788 |
| 199 | 30000 | 2620 |
| 200a | 1223 | 72.89 |
| 200b | | 1125 |
| 201a | | 11110 |
| 201b | 15610 | 45.9 |
| 202a | 11470 | 301.9 |
| 202b | 302 | 3.94 |
| 203a | 3022 | 379.6 |
| 203b | 119.6 | 3.32 |
| 204a | 64.21 | 1.13 |
| 204b | 442.8 | 23.38 |
| 205 | 9382 | 93.94 |
| 206 | 30000 | 1343 |
| 207 | 30000 | 767.3 |
| 208 | 30000 | 210.4 |
| 209 | 2906 | 46.13 |
| 210 | | 2155 |
| 211a | | 2083 |
| 211b | 2009 | 7.412 |
| 212 | 9062 | 179.9 |
| 213 | 25640 | 4460 |
| 214a | 3709 | 4.95 |
| 214b | 9074 | 180.3 |
| 215a | 14020 | 5.06 |
| 215b | 2369 | 429.4 |
| 216a | 3090 | 6.65 |
| 216b | 7118 | 804.4 |
| 217a | 2637 | 12.66 |
| 217b | 8568 | 675.3 |
| 218 | 1553 | 6.54 |
| 219a | 1249 | 3.27 |
| 219b | 9385 | 627.3 |
| 220 | 7827 | 470.4 |
| 221 | | 1106 |
| 222 | 2880 | 8.84 |
| 223 | 27300 | 424.7 |
| 225 | | 69.85 |
| 226 | | 55.14 |
| 227a | 1515 | 190.4 |
| 227b | 32.56 | 1.024 |
| 228b | 64.44 | 2.43 |
| 229 | 1737 | 98.5 |
| 230a | 1446 | 30.87 |
| 230b | 9101 | 161.6 |
| 231a | 9301 | 1490 |
| 231b | 325.9 | 38.44 |
| 232a | 30000 | 932.2 |
| 232b | 154.7 | 5.90 |
| 233a | | 1074 |
| 233b | | 6.11 |
| 234 | 784.7 | 63.96 |
| 235 | 909.6 | 91.7 |
| 236 | 68.73 | 3.11 |
| 237 | 508.3 | 57.62 |
| 238a | 1587 | 25.75 |
| 238b | 21070 | 2709 |
| 240 | | 134.9 |
| 241 | 1758 | 8.63 |
| 242a | 134.8 | 1.15 |
| 242b | 1084 | 93.45 |
| 243a | 30000 | 345.9 |
| 243b | 20460 | 23.05 |
| 244 | 161 | 3.70 |
| 245 | 193.7 | 2.23 |
| 246a | | 41.29 |
| 246b | | 165.3 |
| 247a | 7924 | 101.2 |
| 247b | 152.2 | 0.98 |
| 248a | 1149 | 6.46 |
| 248b | 9718 | 1397 |
| 249a | | 351.4 |
| 249b | 359.5 | 1.52 |
| 250a | | 400.1 |
| 250b | 63.12 | 1.54 |
| 251b | | 934.8 |
| 251a | | 5.15 |
| 252a | 64.28 | 0.85 |
| 252b | | 204.4 |
| 253 | 111.4 | 1.74 |
| 254 | 80.34 | 0.77 |
| 255 | 58.86 | 0.79 |
| 256 | 5873 | 246.4 |

Sequence
(SEQ ID NO: 1)
GLNDIFEAQKIEWHETEYKLVVVGADGVGKSALTIQLIQNHFVDEY
DPTIEDSYRKQVVIDGETCLLDILDTAGQEEYSAMRDQYMRTGEG
FLCVFAINNTKSFEDIHHYREQIKRVKDSEDVPMVLVGNKCDLPS
RTVDTKQAQDLARSYGIPFIETSAKTRQGVDDAFYTLVREIRKHK
EK

1. A compound selected from the group consisting of: 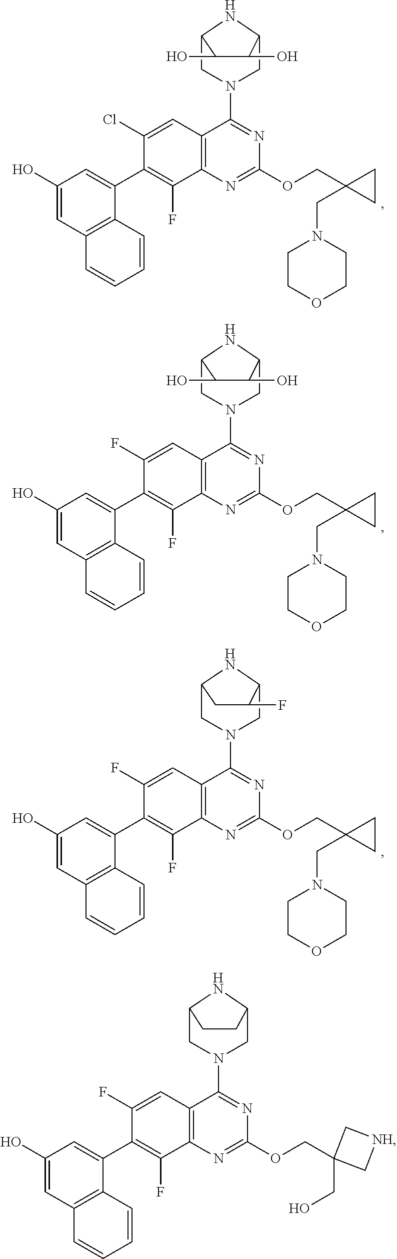 [SMILES: Oc1cc(-c2c(Cl)cc3c(N4CC5NC(C4)C(O)C5O)nc(OCC4(CN5CCOCC5)CC4)nc3c2F)c2ccccc2c1 ; Oc1cc(-c2c(F)cc3c(N4CC5NC(C4)C(O)C5O)nc(OCC4(CN5CCOCC5)CC4)nc3c2F)c2ccccc2c1 ; Oc1cc(-c2c(F)cc3c(N4CC5CC(F)C(C4)N5)nc(OCC4(CN5CCOCC5)CC4)nc3c2F)c2ccccc2c1 ; OCC1(COc2nc(N3CC4CCC(C3)N4)c3cc(F)c(-c4cc(O)cc5ccccc45)c(F)c3n2)CNC1] 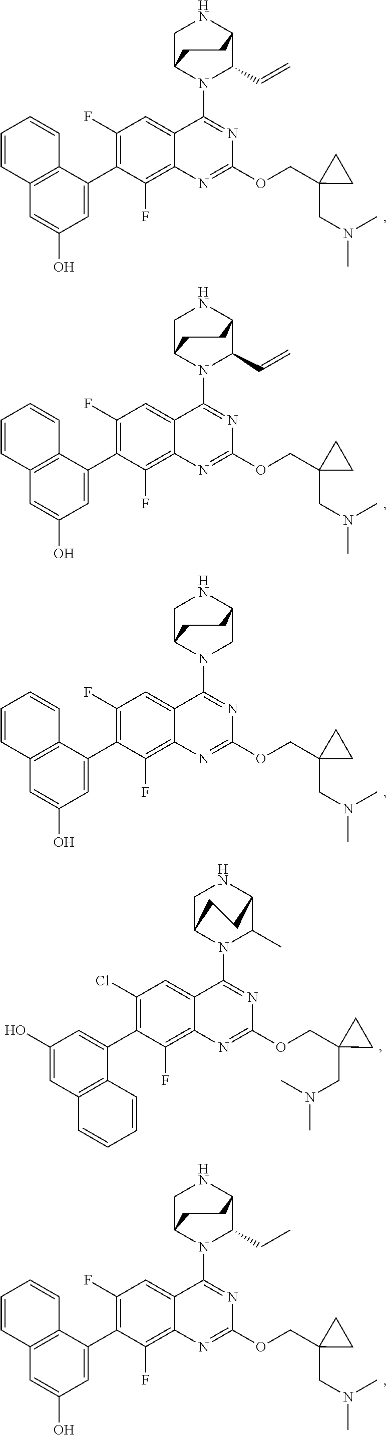 [SMILES: C=C[C@H]1[C@@H]2CC[C@@H](CN2)N1c1nc(OCC2(CN(C)C)CC2)nc2c(F)c(-c3cc(O)cc4ccccc34)c(F)cc12 ; C=C[C@@H]1[C@@H]2CC[C@@H](CN2)N1c1nc(OCC2(CN(C)C)CC2)nc2c(F)c(-c3cc(O)cc4ccccc34)c(F)cc12 ; CN(C)CC1(COc2nc(N3C[C@@H]4CC[C@H]3CN4)c3cc(F)c(-c4cc(O)cc5ccccc45)c(F)c3n2)CC1 ; CC1[C@@H]2CC[C@@H](CN2)N1c1nc(OCC2(CN(C)C)CC2)nc2c(F)c(-c3cc(O)cc4ccccc34)c(Cl)cc12 ; CC[C@H]1[C@@H]2CC[C@@H](CN2)N1c1nc(OCC2(CN(C)C)CC2)nc2c(F)c(-c3cc(O)cc4ccccc34)c(F)cc12] 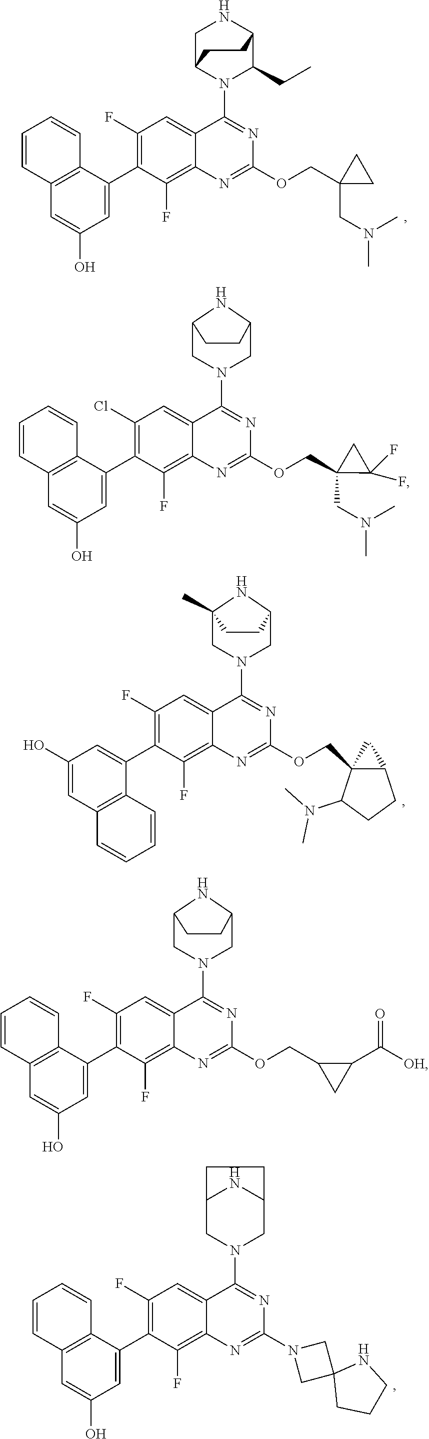 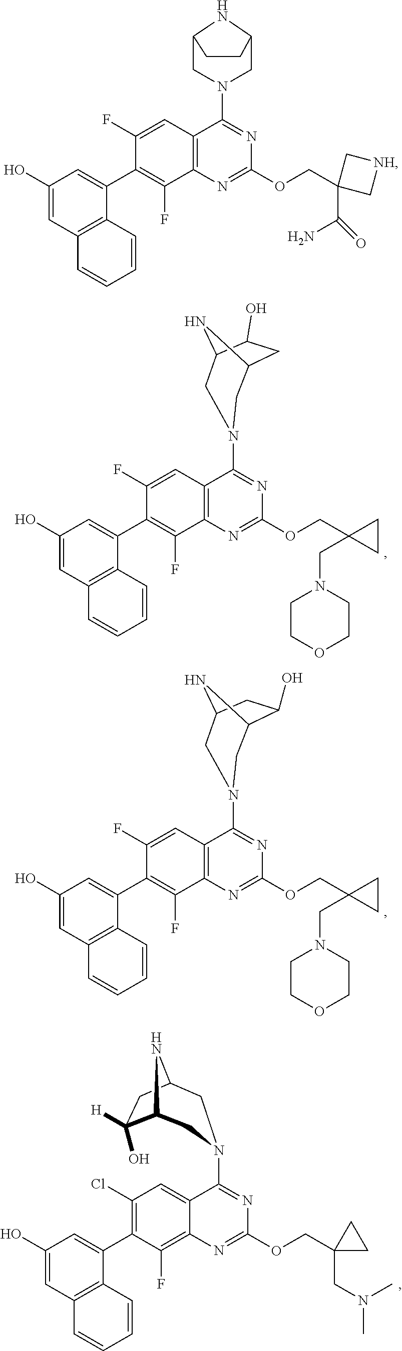 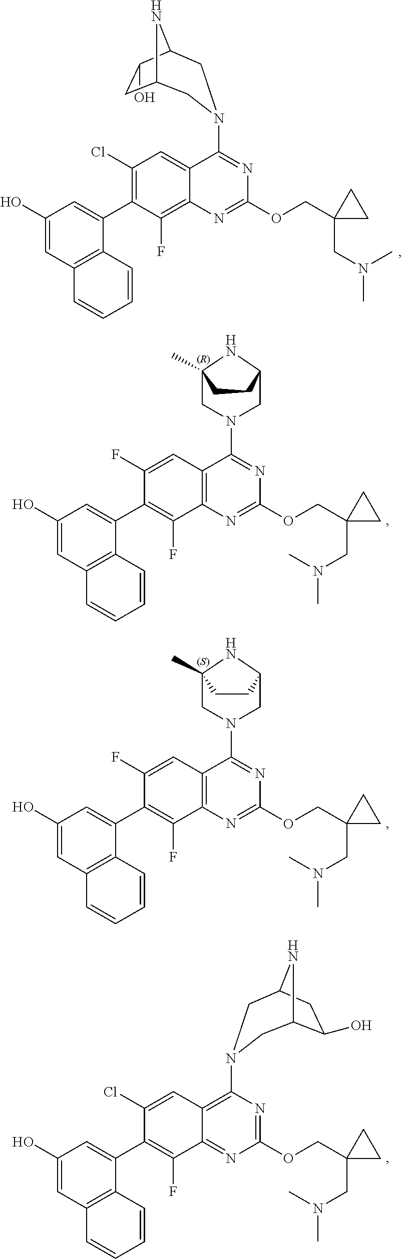 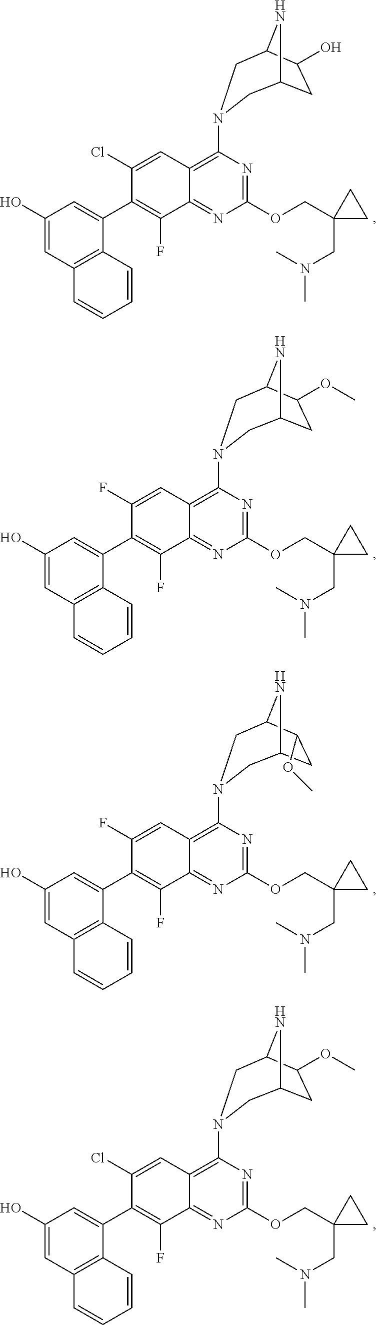 [SMILES: CN(C)CC1(COc2nc(N3CC4CC(O)C(C3)N4)c3cc(Cl)c(-c4cc(O)cc5ccccc45)c(F)c3n2)CC1 ; COC1CC2CN(c3nc(OCC4(CN(C)C)CC4)nc4c(F)c(-c5cc(O)cc6ccccc56)c(F)cc34)CC1N2 ; COC1CC2CN(c3nc(OCC4(CN(C)C)CC4)nc4c(F)c(-c5cc(O)cc6ccccc56)c(F)cc34)CC1N2 ; COC1CC2CN(c3nc(OCC4(CN(C)C)CC4)nc4c(F)c(-c5cc(O)cc6ccccc56)c(Cl)cc34)CC1N2] 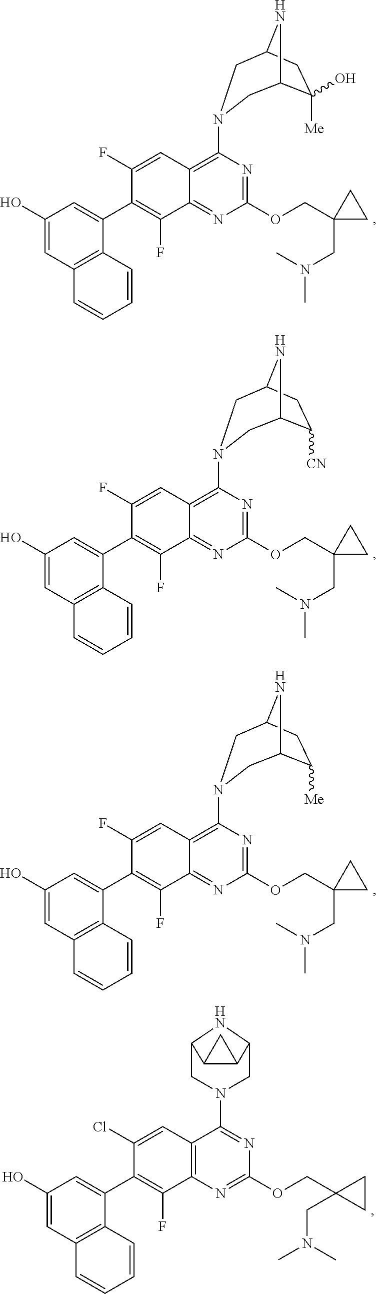 [SMILES: CN(C)CC1(COc2nc(N3CC4CC(C)(O)C(C3)N4)c3cc(F)c(-c4cc(O)cc5ccccc45)c(F)c3n2)CC1 ; CN(C)CC1(COc2nc(N3CC4CC(C#N)C(C3)N4)c3cc(F)c(-c4cc(O)cc5ccccc45)c(F)c3n2)CC1 ; CC1CC2CN(c3nc(OCC4(CN(C)C)CC4)nc4c(F)c(-c5cc(O)cc6ccccc56)c(F)cc34)CC1N2 ; CN(C)CC1(COc2nc(N3CC4NC(C3)C3CC43)c3cc(Cl)c(-c4cc(O)cc5ccccc45)c(F)c3n2)CC1] 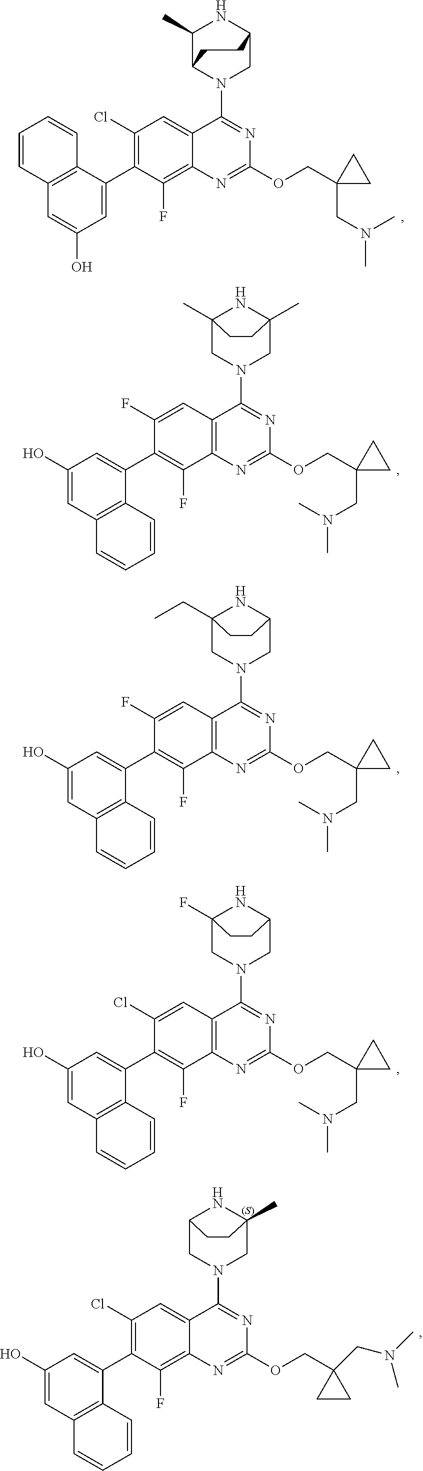 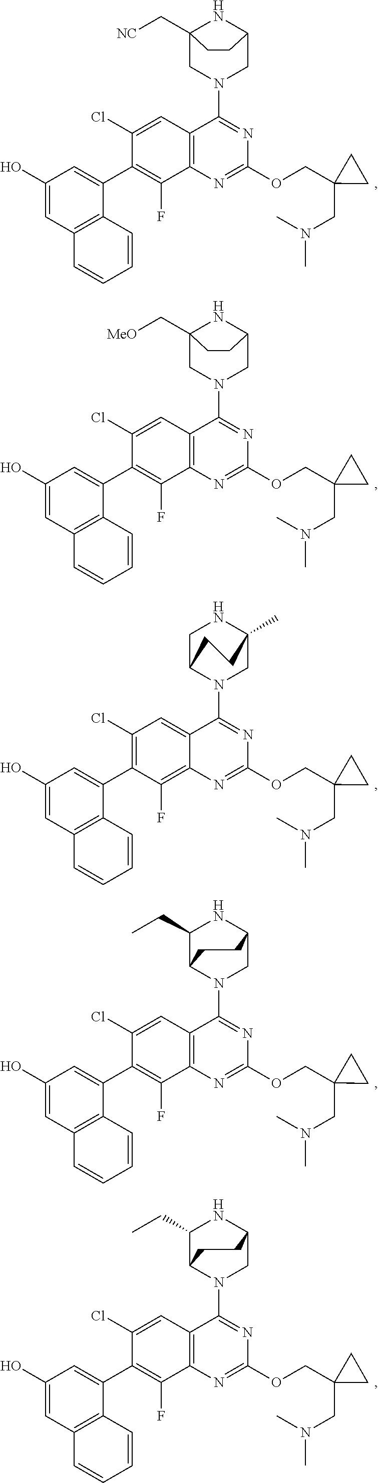 [SMILES: CN(C)CC1(COc2nc(N3CC4CCC(CC#N)(C3)N4)c3cc(Cl)c(-c4cc(O)cc5ccccc45)c(F)c3n2)CC1 ; COCC12CCC(CN(c3nc(OCC4(CN(C)C)CC4)nc4c(F)c(-c5cc(O)cc6ccccc56)c(Cl)cc34)C1)N2 ; CN(C)CC1(COc2nc(N3C[C@]4(C)CC[C@H]3CN4)c3cc(Cl)c(-c4cc(O)cc5ccccc45)c(F)c3n2)CC1 ; CC[C@H]1N[C@H]2CC[C@@H]1N(c1nc(OCC3(CN(C)C)CC3)nc3c(F)c(-c4cc(O)cc5ccccc45)c(Cl)cc13)C2 ; CC[C@@H]1N[C@H]2CC[C@@H]1N(c1nc(OCC3(CN(C)C)CC3)nc3c(F)c(-c4cc(O)cc5ccccc45)c(Cl)cc13)C2] 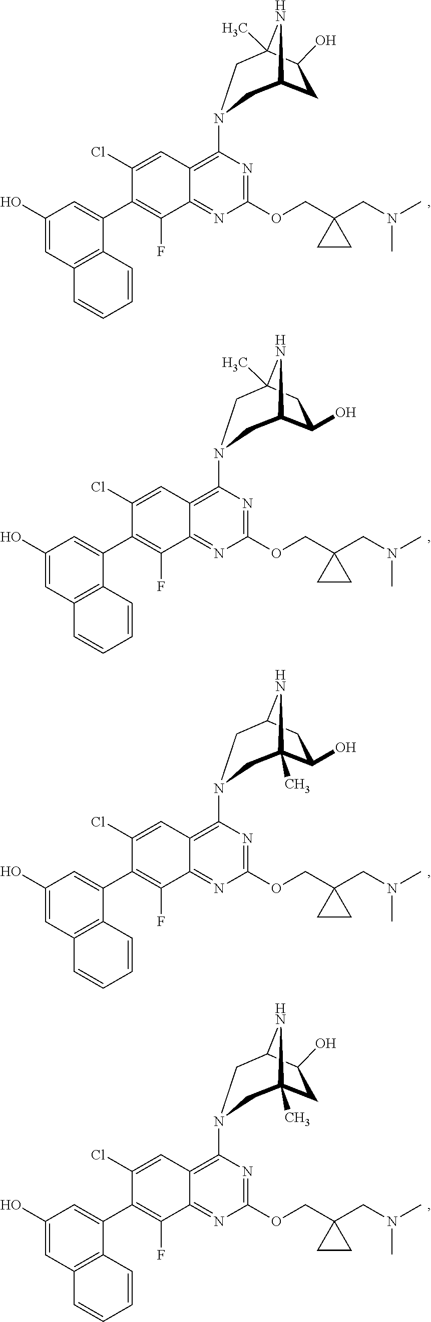 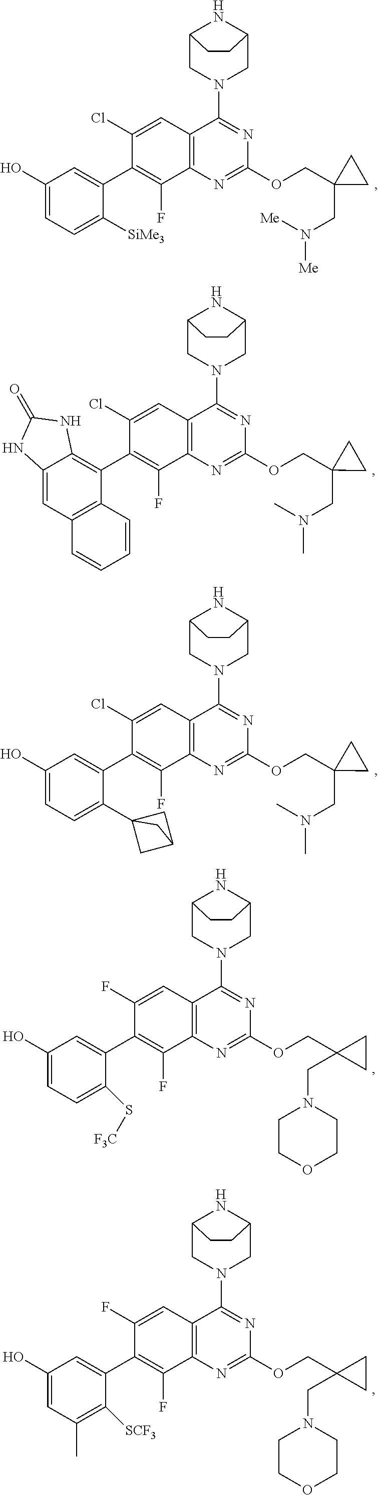 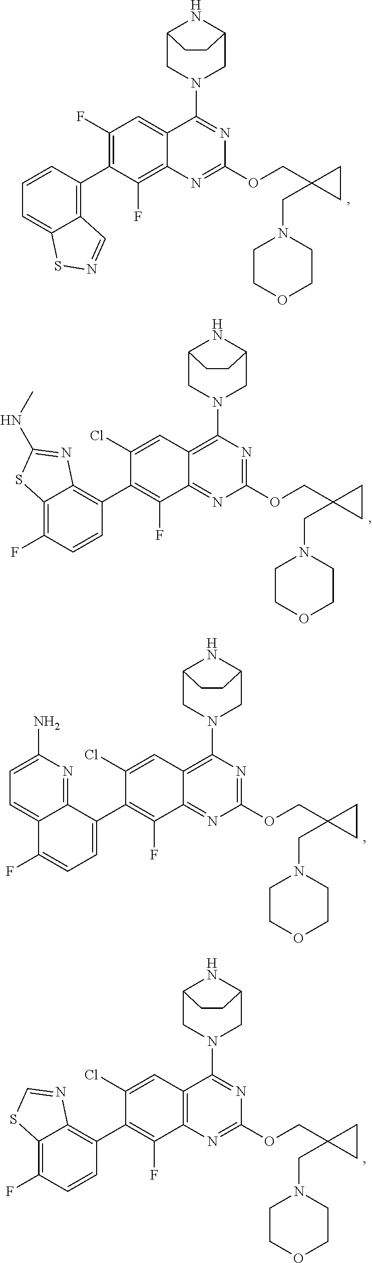 [SMILES: Fc1cc2c(N3CC4CCC(C3)N4)nc(OCC3(CN4CCOCC4)CC3)nc2c(F)c1-c1cccc2sncc12 ; CNc1nc2c(-c3c(Cl)cc4c(N5CC6CCC(C5)N6)nc(OCC5(CN6CCOCC6)CC5)nc4c3F)ccc(F)c2s1 ; Nc1ccc2c(F)ccc(-c3c(Cl)cc4c(N5CC6CCC(C5)N6)nc(OCC5(CN6CCOCC6)CC5)nc4c3F)c2n1 ; Fc1c(-c2ccc(F)c3scnc23)c(Cl)cc2c(N3CC4CCC(C3)N4)nc(OCC3(CN4CCOCC4)CC3)nc12] 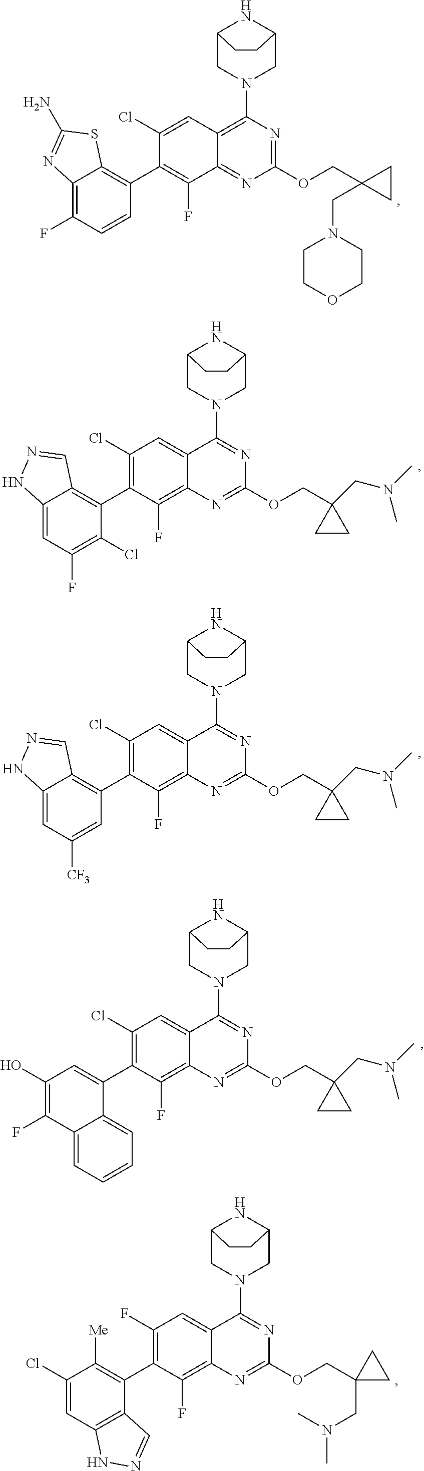 [SMILES: Nc1nc2c(F)ccc(-c3c(Cl)cc4c(N5CC6CCC(C5)N6)nc(OCC5(CN6CCOCC6)CC5)nc4c3F)c2s1 ; CN(C)CC1(COc2nc(N3CC4CCC(C3)N4)c3cc(Cl)c(-c4c(Cl)c(F)cc5[nH]ncc45)c(F)c3n2)CC1 ; CN(C)CC1(COc2nc(N3CC4CCC(C3)N4)c3cc(Cl)c(-c4cc(C(F)(F)F)cc5[nH]ncc45)c(F)c3n2)CC1 ; CN(C)CC1(COc2nc(N3CC4CCC(C3)N4)c3cc(Cl)c(-c4cc(O)c(F)c5ccccc45)c(F)c3n2)CC1 ; Cc1c(Cl)cc2[nH]ncc2c1-c1c(F)cc2c(N3CC4CCC(C3)N4)nc(OCC3(CN(C)C)CC3)nc2c1F] 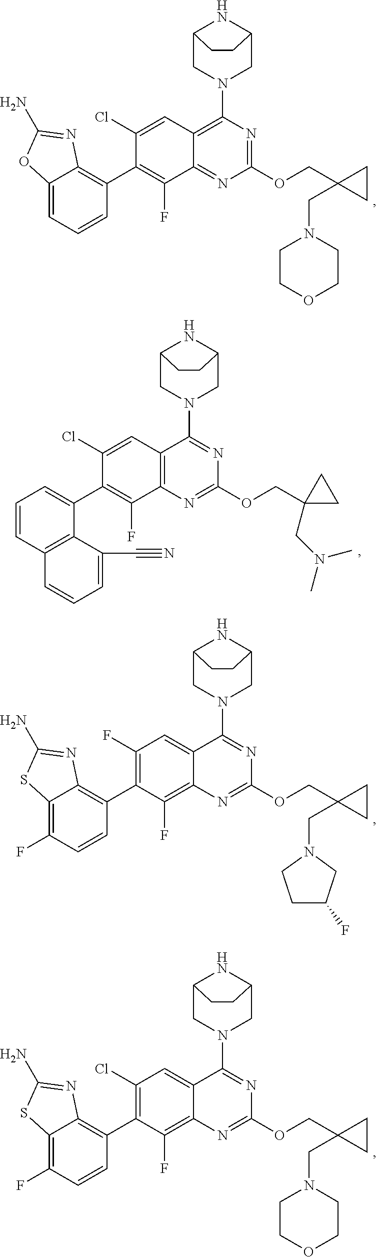 [SMILES: Nc1nc2c(-c3c(Cl)cc4c(N5CC6CCC(C5)N6)nc(OCC5(CN6CCOCC6)CC5)nc4c3F)cccc2o1 ; CN(C)CC1(COc2nc(N3CC4CCC(C3)N4)c3cc(Cl)c(-c4cccc5cccc(C#N)c45)c(F)c3n2)CC1 ; Nc1nc2c(-c3c(F)cc4c(N5CC6CCC(C5)N6)nc(OCC5(CN6CC[C@@H](F)C6)CC5)nc4c3F)ccc(F)c2s1 ; Nc1nc2c(-c3c(Cl)cc4c(N5CC6CCC(C5)N6)nc(OCC5(CN6CCOCC6)CC5)nc4c3F)ccc(F)c2s1] 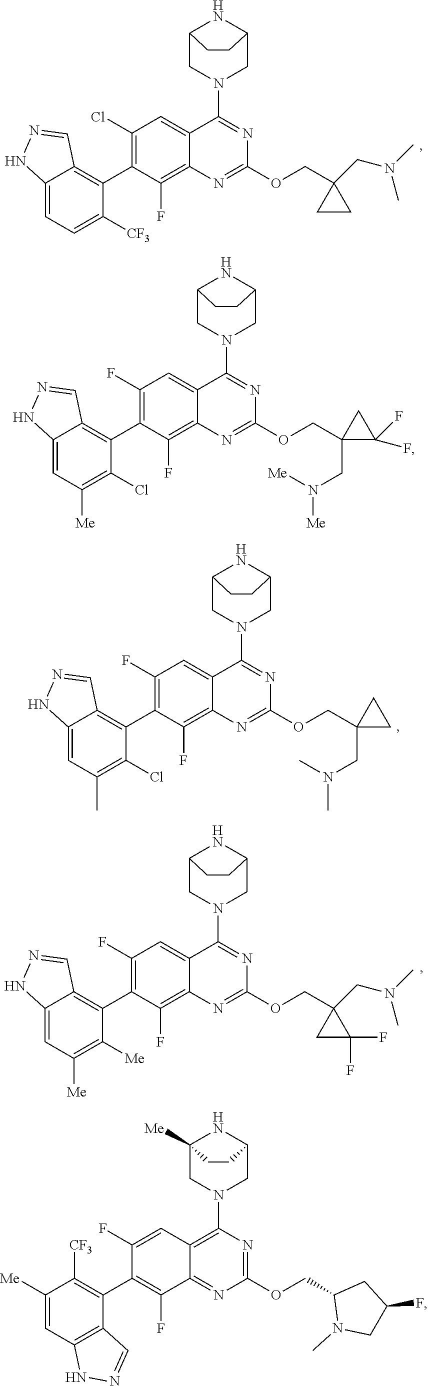 [SMILES: CN(C)CC1(COc2nc(N3CC4CCC(C3)N4)c3cc(Cl)c(-c4c(C(F)(F)F)ccc5[nH]ncc45)c(F)c3n2)CC1 ; Cc1cc2[nH]ncc2c(-c2c(F)cc3c(N4CC5CCC(C4)N5)nc(OCC4(CN(C)C)CC4(F)F)nc3c2F)c1Cl ; Cc1cc2[nH]ncc2c(-c2c(F)cc3c(N4CC5CCC(C4)N5)nc(OCC4(CN(C)C)CC4)nc3c2F)c1Cl ; Cc1cc2[nH]ncc2c(-c2c(F)cc3c(N4CC5CCC(C4)N5)nc(OCC4(CN(C)C)CC4(F)F)nc3c2F)c1C ; Cc1cc2[nH]ncc2c(-c2c(F)cc3c(N4C[C@H]5CC[C@@](C)(C4)N5)nc(OC[C@@H]4C[C@@H](F)CN4C)nc3c2F)c1C(F)(F)F] 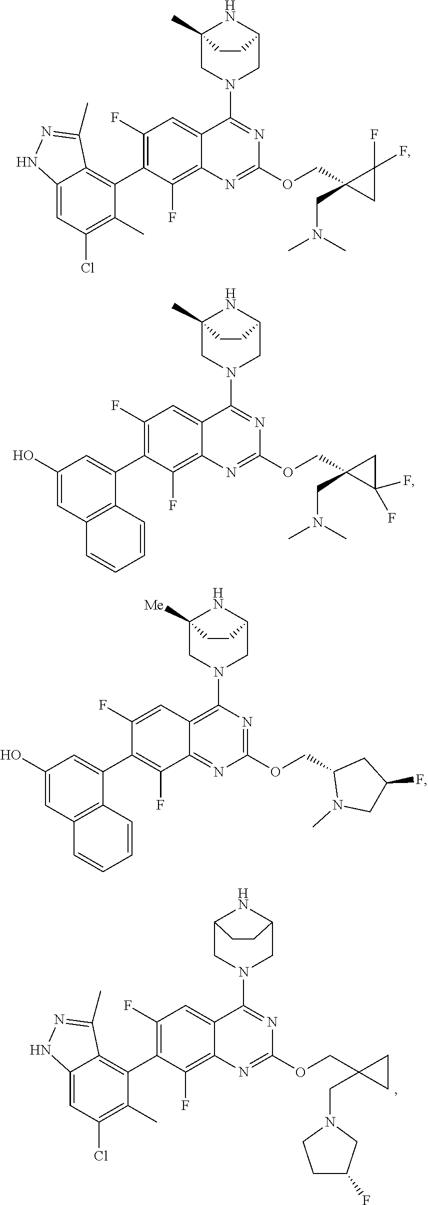 [SMILES: Cc1c(Cl)cc2[nH]nc(C)c2c1-c1c(F)cc2c(N3C[C@H]4CC[C@@](C)(C3)N4)nc(OC[C@@]3(CN(C)C)CC3(F)F)nc2c1F ; CN(C)C[C@@]1(COc2nc(N3C[C@H]4CC[C@@](C)(C3)N4)c3cc(F)c(-c4cc(O)cc5ccccc45)c(F)c3n2)CC1(F)F ; CN1C[C@H](F)C[C@H]1COc1nc(N2C[C@H]3CC[C@@](C)(C2)N3)c2cc(F)c(-c3cc(O)cc4ccccc34)c(F)c2n1 ; Cc1c(Cl)cc2[nH]nc(C)c2c1-c1c(F)cc2c(N3CC4CCC(C3)N4)nc(OCC3(CN4CC[C@@H](F)C4)CC3)nc2c1F] 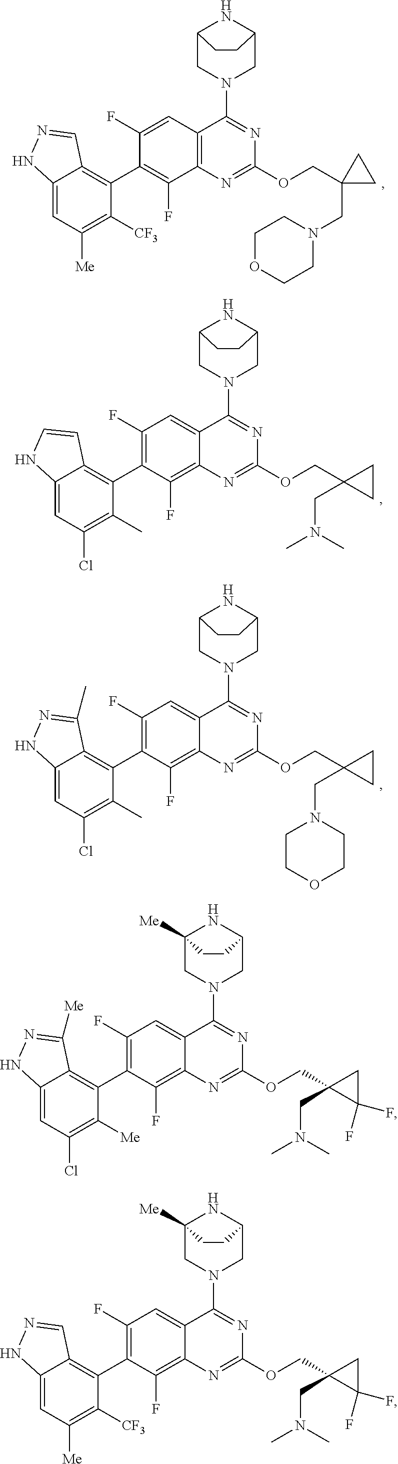 [SMILES: Cc1cc2[nH]ncc2c(-c2c(F)cc3c(N4CC5CCC(C4)N5)nc(OCC4(CN5CCOCC5)CC4)nc3c2F)c1C(F)(F)F ; Cc1c(Cl)cc2[nH]ccc2c1-c1c(F)cc2c(N3CC4CCC(C3)N4)nc(OCC3(CN(C)C)CC3)nc2c1F ; Cc1c(Cl)cc2[nH]nc(C)c2c1-c1c(F)cc2c(N3CC4CCC(C3)N4)nc(OCC3(CN4CCOCC4)CC3)nc2c1F ; Cc1c(Cl)cc2[nH]nc(C)c2c1-c1c(F)cc2c(N3C[C@H]4CC[C@@](C)(C3)N4)nc(OC[C@]3(CN(C)C)CC3(F)F)nc2c1F ; Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C(F)(F)F] 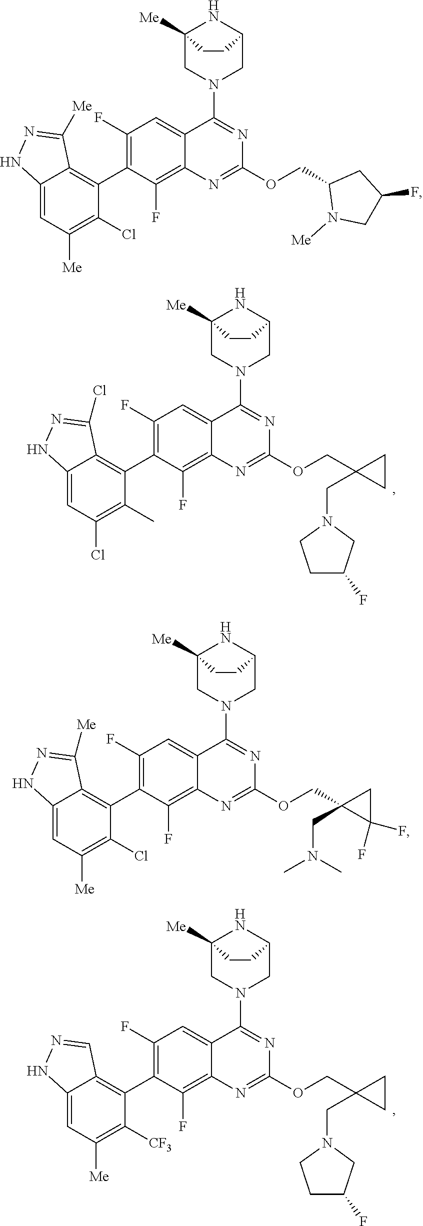 [SMILES: Cc1cc2[nH]nc(C)c2c(-c2c(F)cc3c(N4C[C@H]5CC[C@@](C)(C4)N5)nc(OC[C@@H]4C[C@@H](F)CN4C)nc3c2F)c1Cl ; Cc1c(Cl)cc2[nH]nc(Cl)c2c1-c1c(F)cc2c(N3C[C@H]4CC[C@@](C)(C3)N4)nc(OCC3(CN4CC[C@@H](F)C4)CC3)nc2c1F ; Cc1cc2[nH]nc(C)c2c(-c2c(F)cc3c(N4C[C@H]5CC[C@@](C)(C4)N5)nc(OC[C@]4(CN(C)C)CC4(F)F)nc3c2F)c1Cl ; Cc1cc2[nH]ncc2c(-c2c(F)cc3c(N4C[C@H]5CC[C@@](C)(C4)N5)nc(OCC4(CN5CC[C@@H](F)C5)CC4)nc3c2F)c1C(F)(F)F] 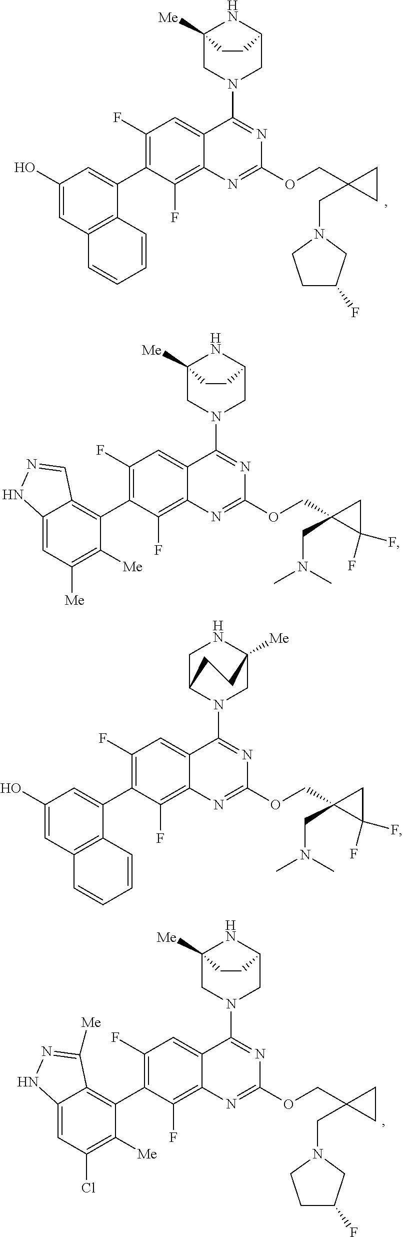 [SMILES: C[C@]12CC[C@H](CN(c3nc(OCC4(CN5CC[C@@H](F)C5)CC4)nc4c(F)c(-c5cc(O)cc6ccccc56)c(F)cc34)C1)N2 ; Cc1cc2[nH]ncc2c(-c2c(F)cc3c(N4C[C@H]5CC[C@@](C)(C4)N5)nc(OC[C@]4(CN(C)C)CC4(F)F)nc3c2F)c1C ; CN(C)C[C@@]1(COc2nc(N3C[C@]4(C)CC[C@H]3CN4)c3cc(F)c(-c4cc(O)cc5ccccc45)c(F)c3n2)CC1(F)F ; Cc1c(Cl)cc2[nH]nc(C)c2c1-c1c(F)cc2c(N3C[C@H]4CC[C@@](C)(C3)N4)nc(OCC3(CN4CC[C@@H](F)C4)CC3)nc2c1F] 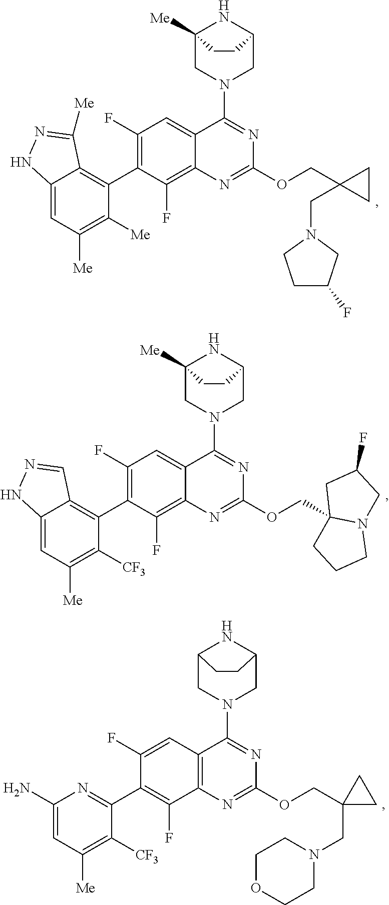 [SMILES: Cc1cc2[nH]nc(C)c2c(-c2c(F)cc3c(N4C[C@H]5CC[C@@](C)(C4)N5)nc(OCC4(CN5CC[C@@H](F)C5)CC4)nc3c2F)c1C ; Cc1cc2[nH]ncc2c(-c2c(F)cc3c(N4C[C@H]5CC[C@@](C)(C4)N5)nc(OC[C@@]45CCCN4C[C@H](F)C5)nc3c2F)c1C(F)(F)F ; Cc1cc(N)nc(-c2c(F)cc3c(N4CC5CCC(C4)N5)nc(OCC4(CN5CCOCC5)CC4)nc3c2F)c1C(F)(F)F] 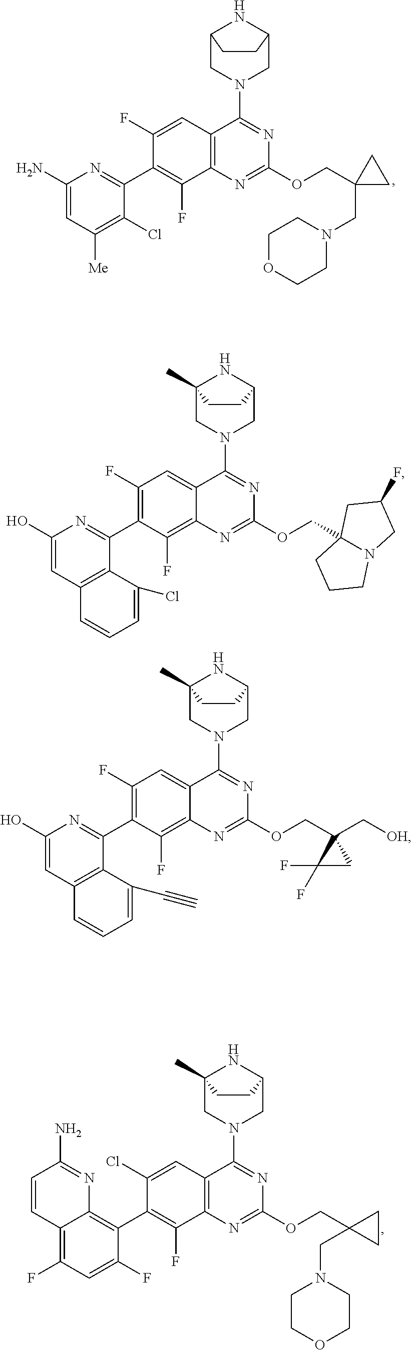 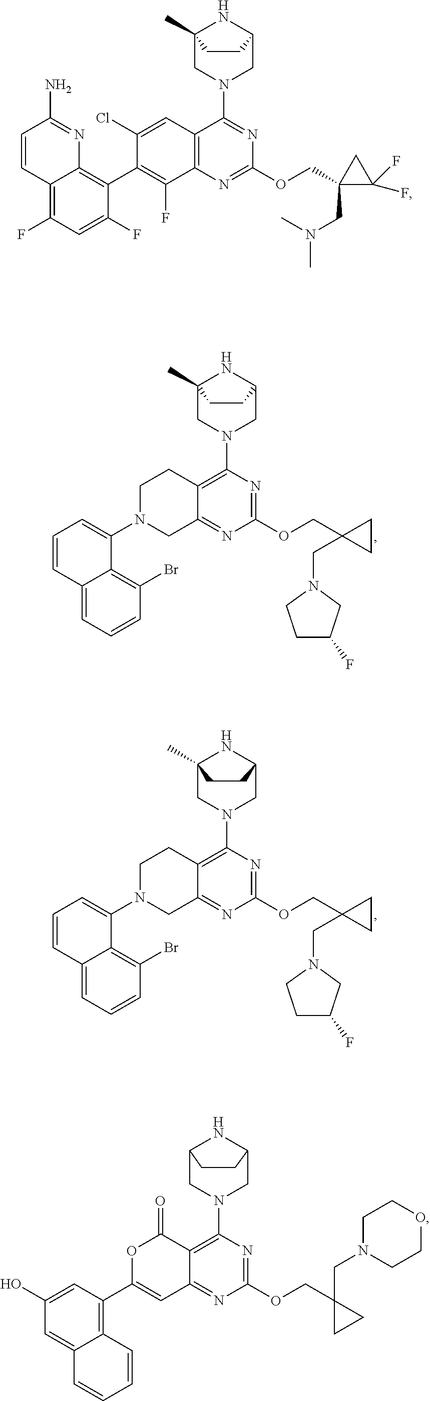 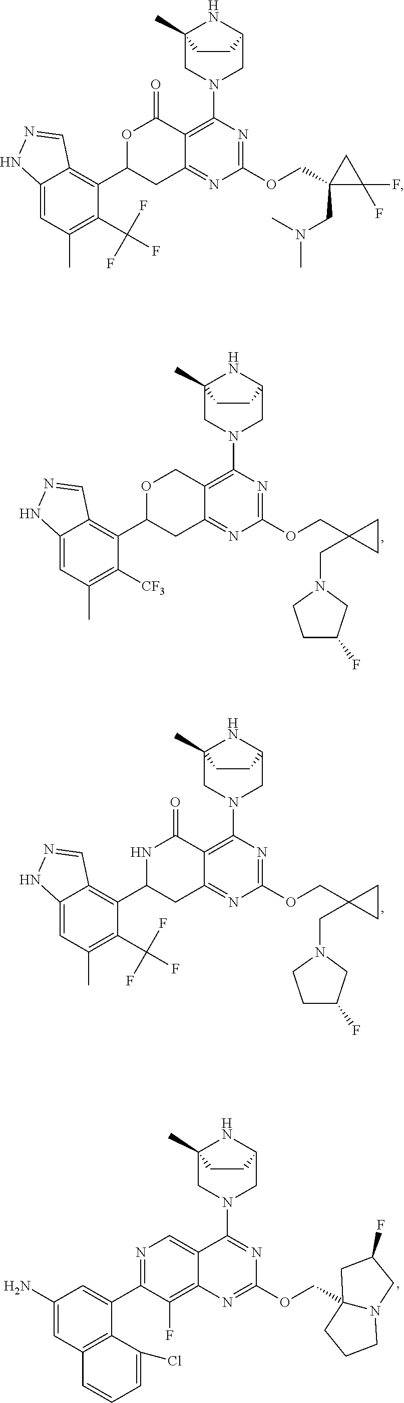 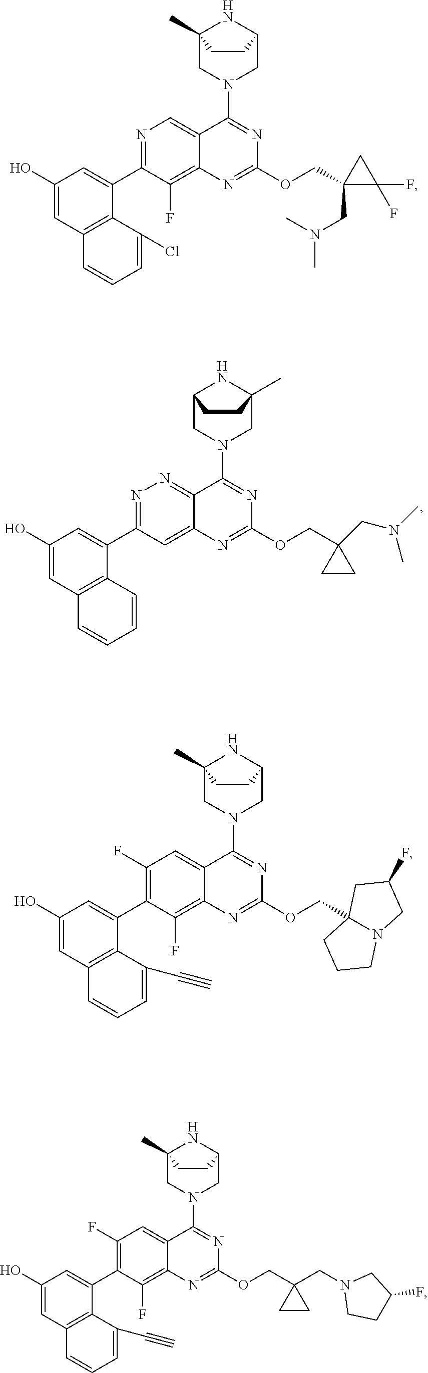 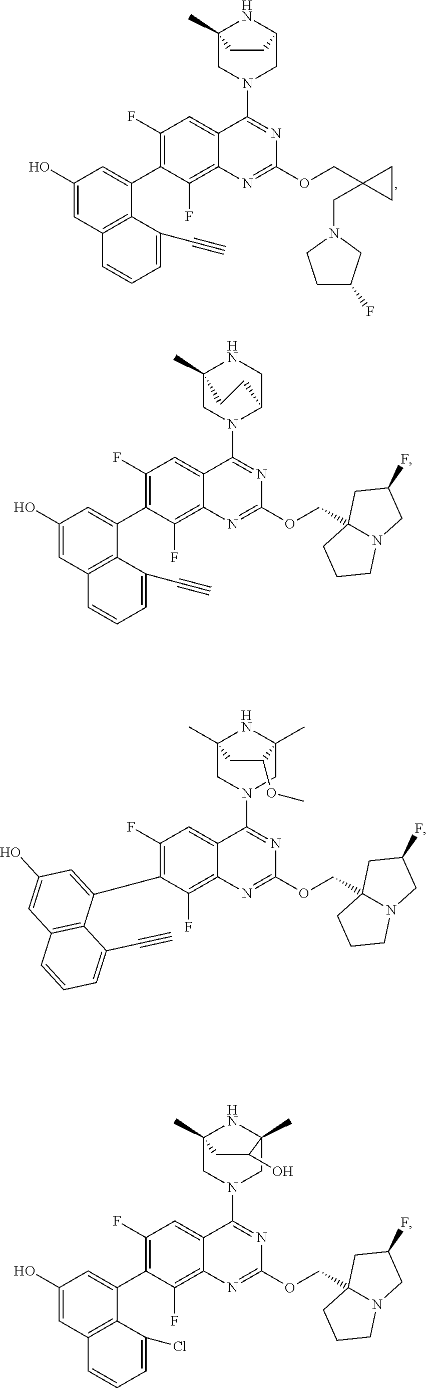 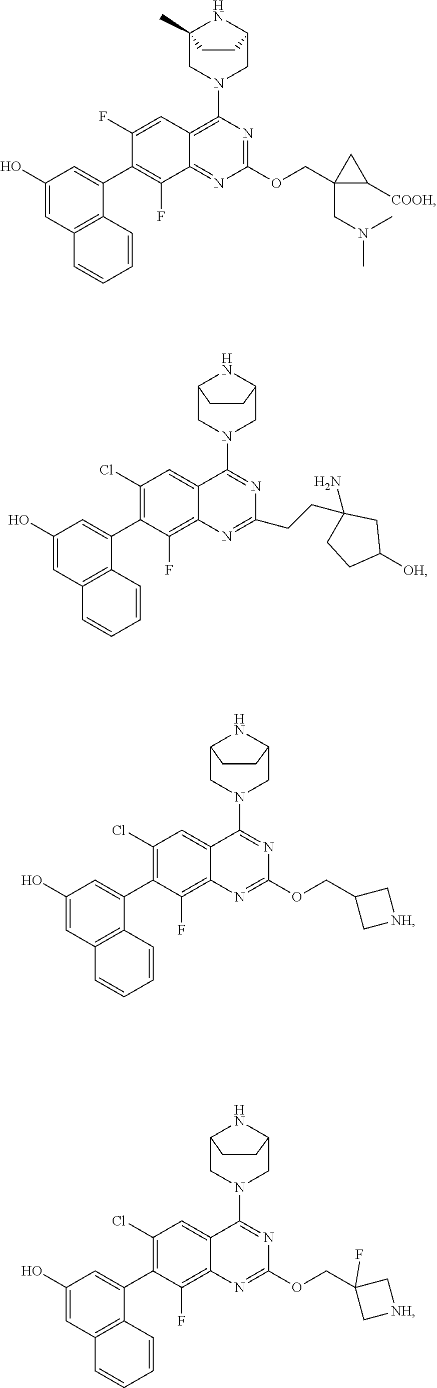 [SMILES: CN(C)CC1(COc2nc(N3C[C@H]4CC[C@@](C)(C3)N4)c3cc(F)c(-c4cc(O)cc5ccccc45)c(F)c3n2)CC1C(=O)O ; NC1(CCc2nc(N3CC4CCC(C3)N4)c3cc(Cl)c(-c4cc(O)cc5ccccc45)c(F)c3n2)CCC(O)C1 ; Oc1cc(-c2c(Cl)cc3c(N4CC5CCC(C4)N5)nc(OCC4CNC4)nc3c2F)c2ccccc2c1 ; Oc1cc(-c2c(Cl)cc3c(N4CC5CCC(C4)N5)nc(OCC4(F)CNC4)nc3c2F)c2ccccc2c1] 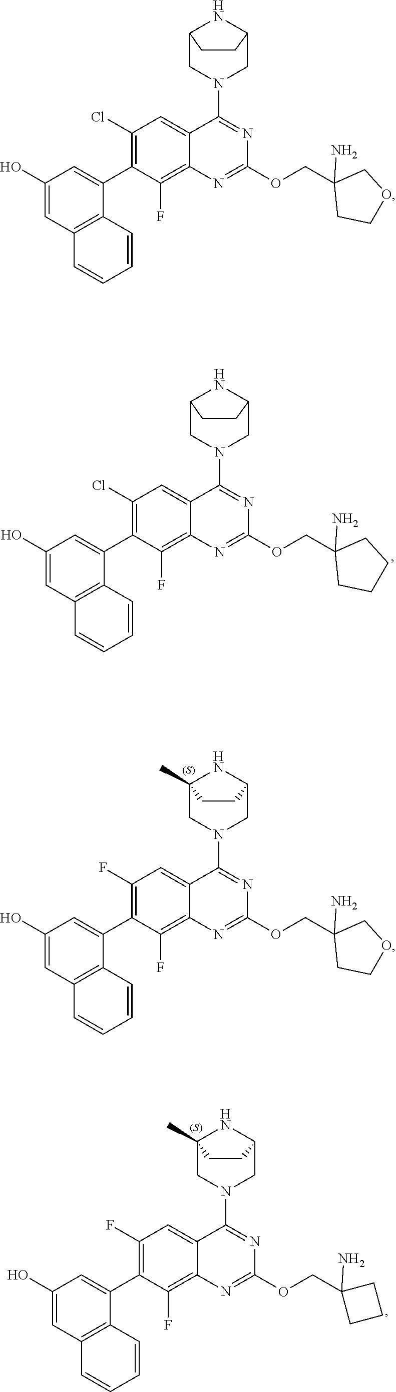 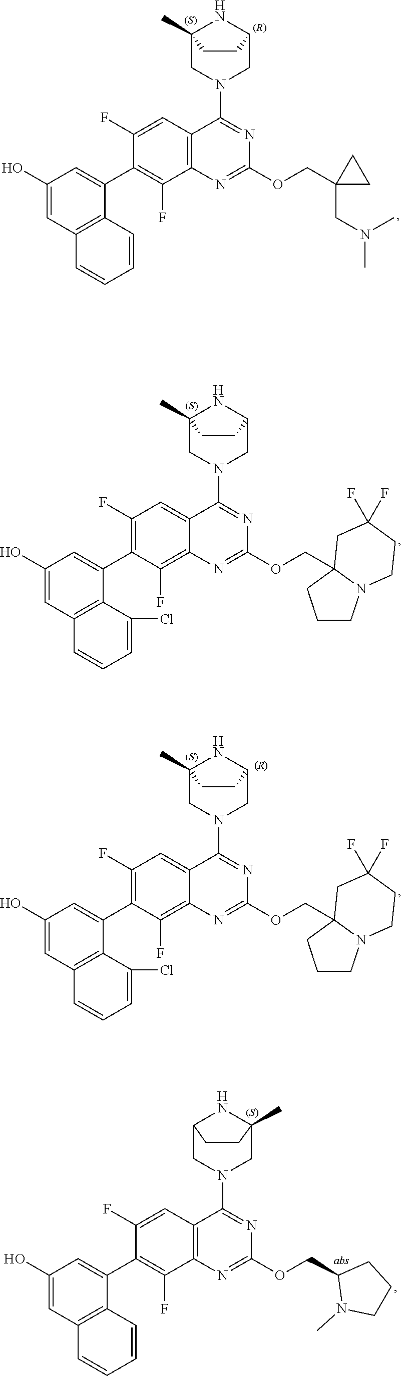 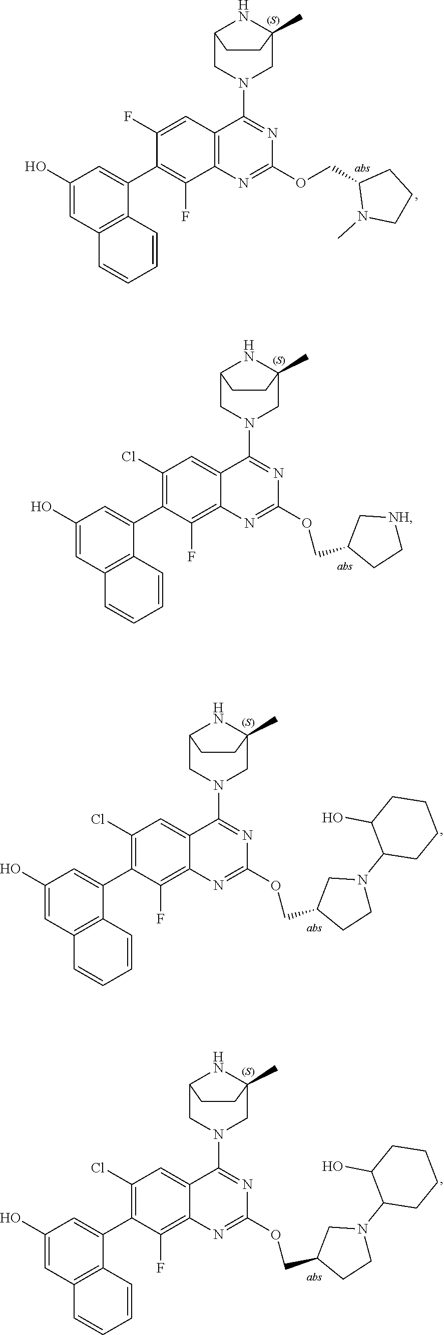 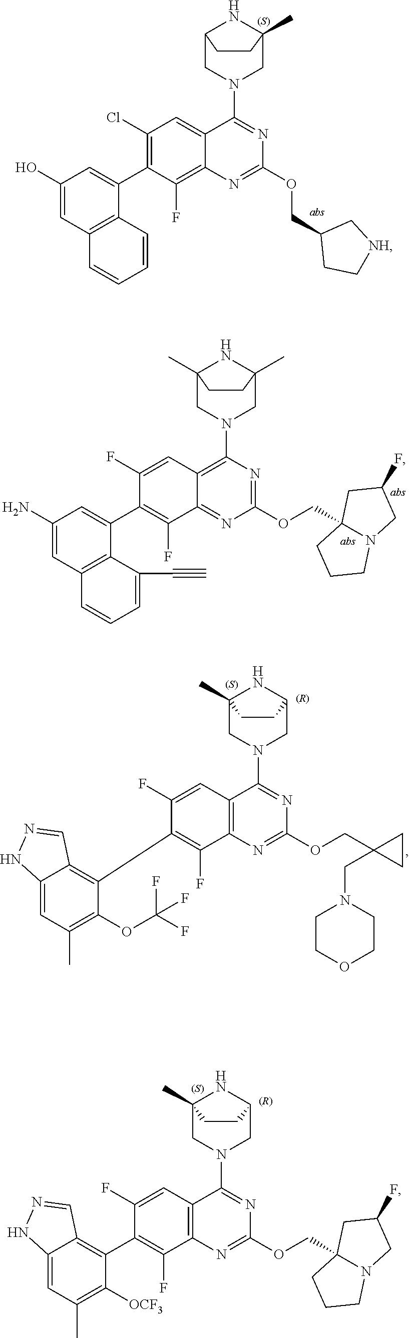 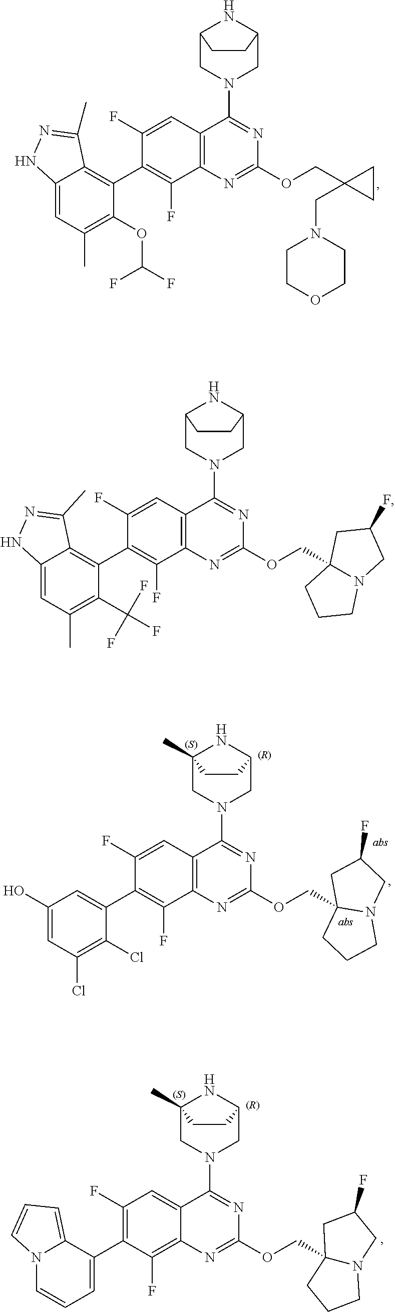 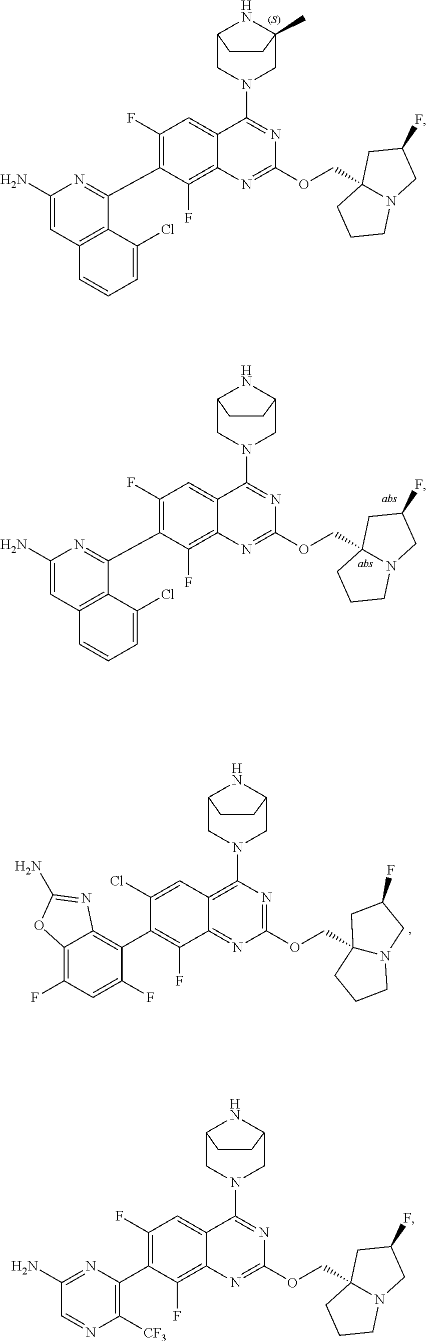 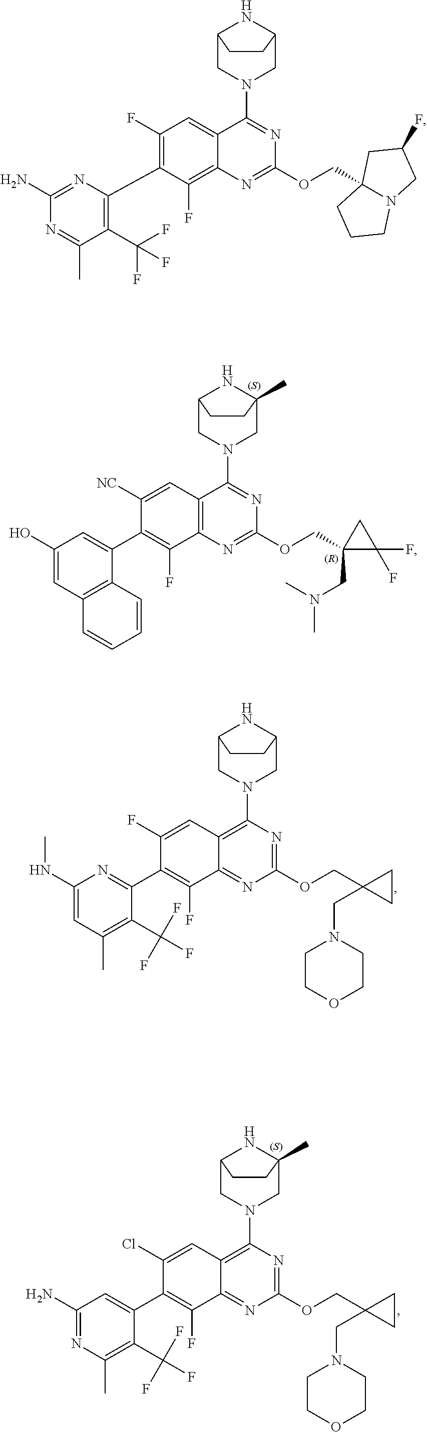 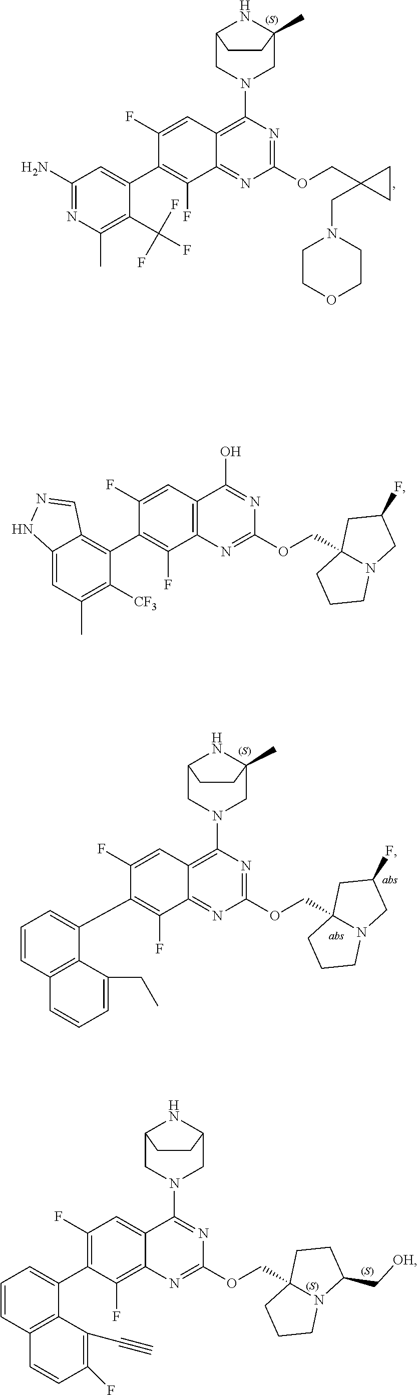 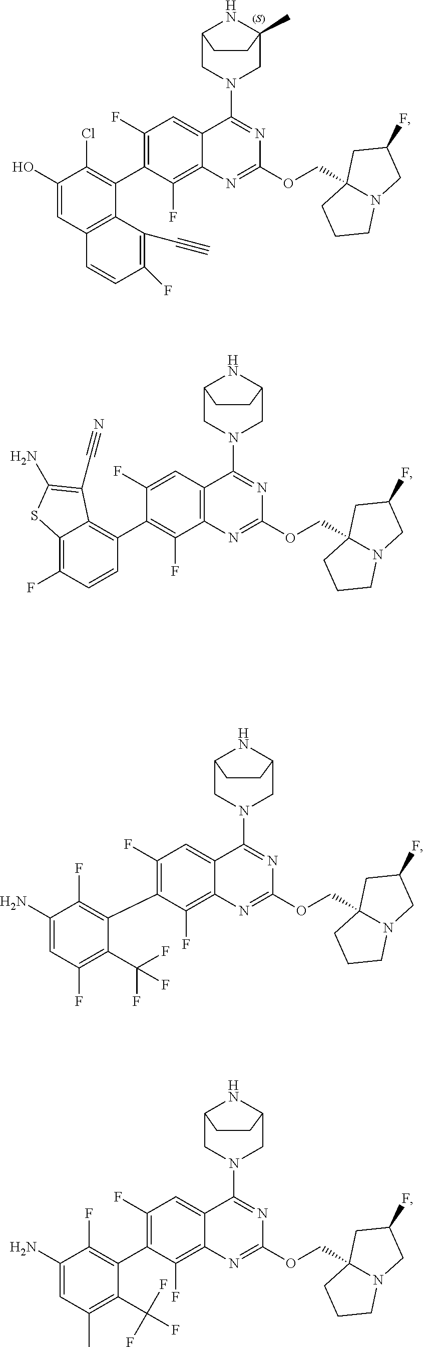 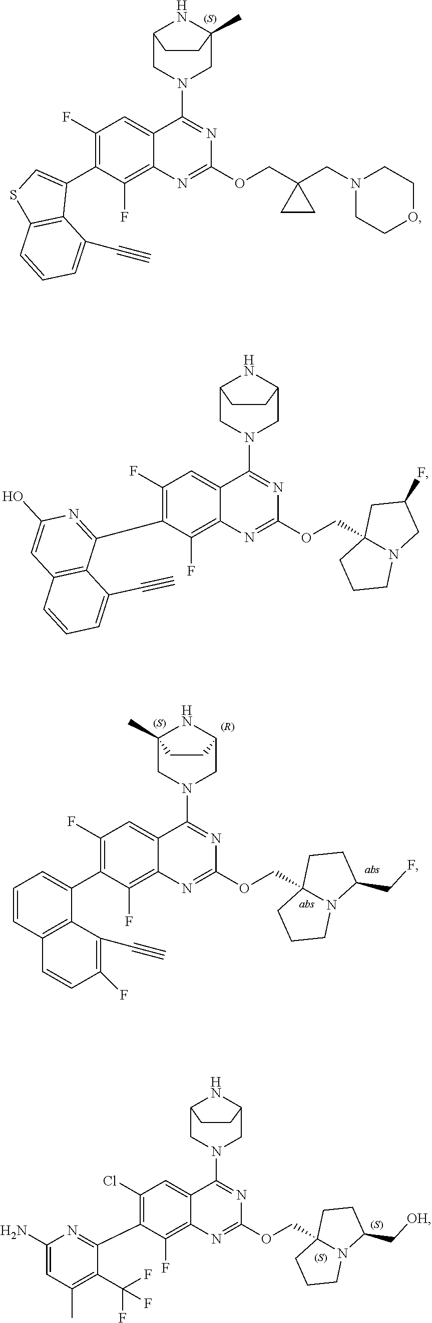 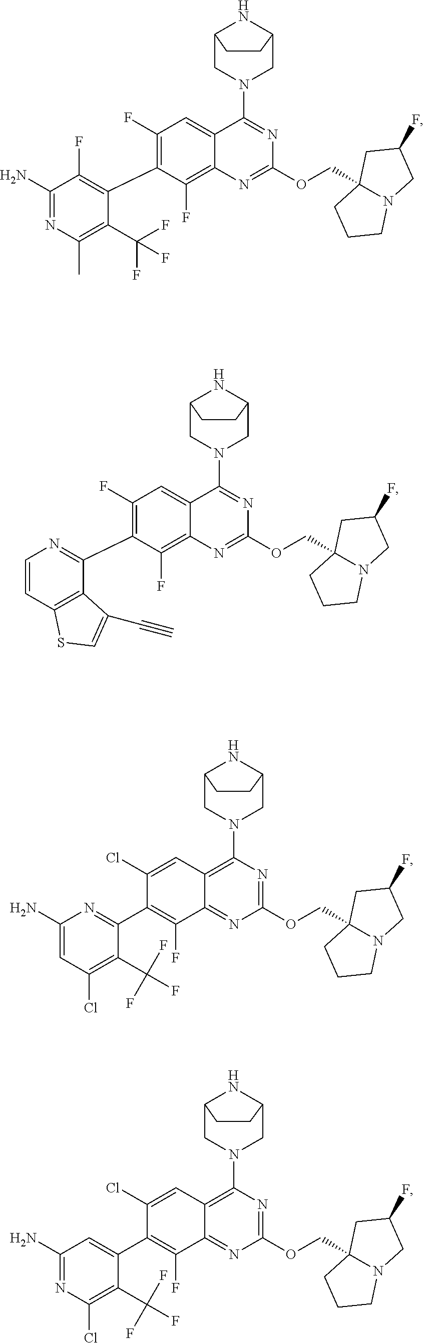 [SMILES: Cc1nc(N)c(F)c(-c2c(F)cc3c(N4CC5CCC(C4)N5)nc(OC[C@@]45CCCN4C[C@H](F)C5)nc3c2F)c1C(F)(F)F ; C#Cc1csc2ccnc(-c3c(F)cc4c(N5CC6CCC(C5)N6)nc(OC[C@@]56CCCN5C[C@H](F)C6)nc4c3F)c12 ; Nc1cc(Cl)c(C(F)(F)F)c(-c2c(Cl)cc3c(N4CC5CCC(C4)N5)nc(OC[C@@]45CCCN4C[C@H](F)C5)nc3c2F)n1 ; Nc1cc(-c2c(Cl)cc3c(N4CC5CCC(C4)N5)nc(OC[C@@]45CCCN4C[C@H](F)C5)nc3c2F)c(C(F)(F)F)c(Cl)n1] 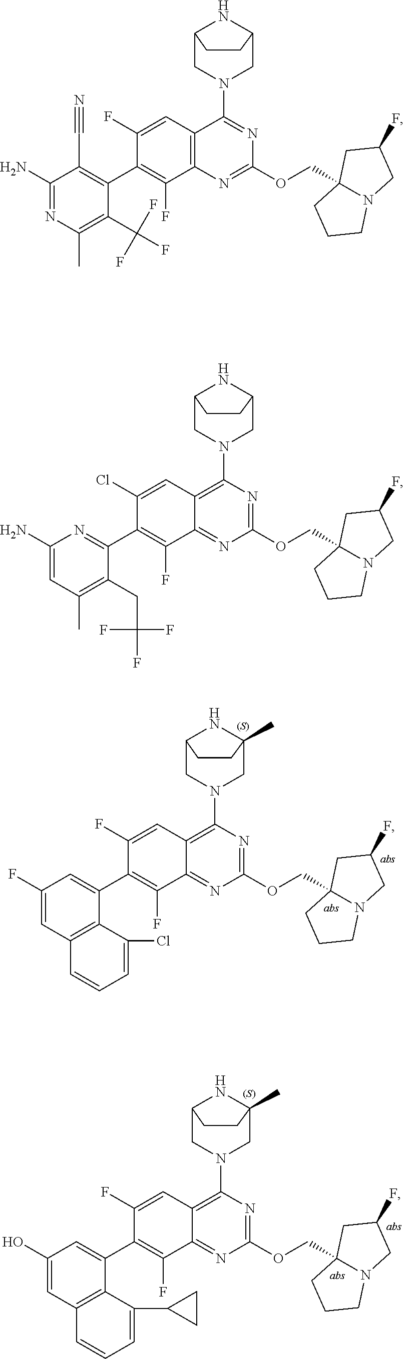 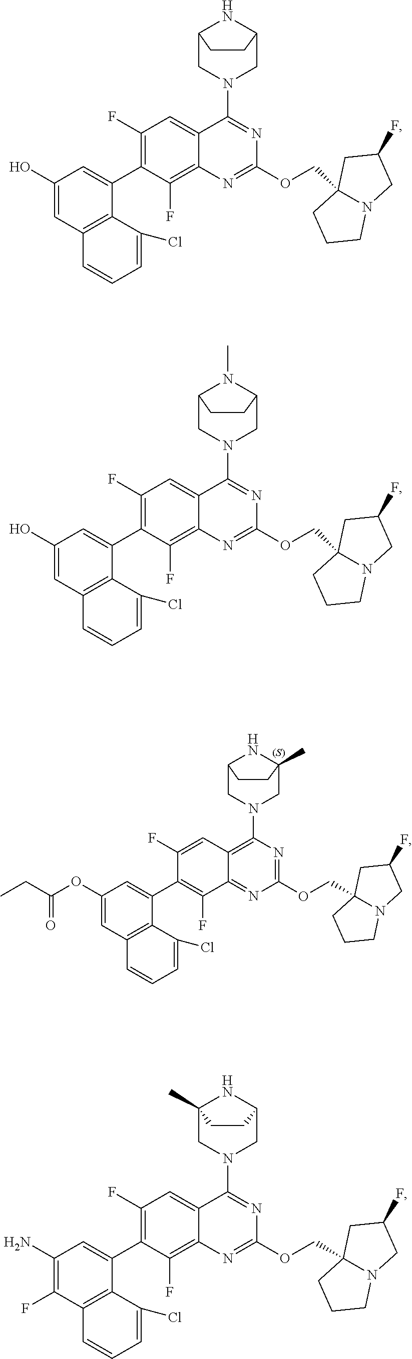 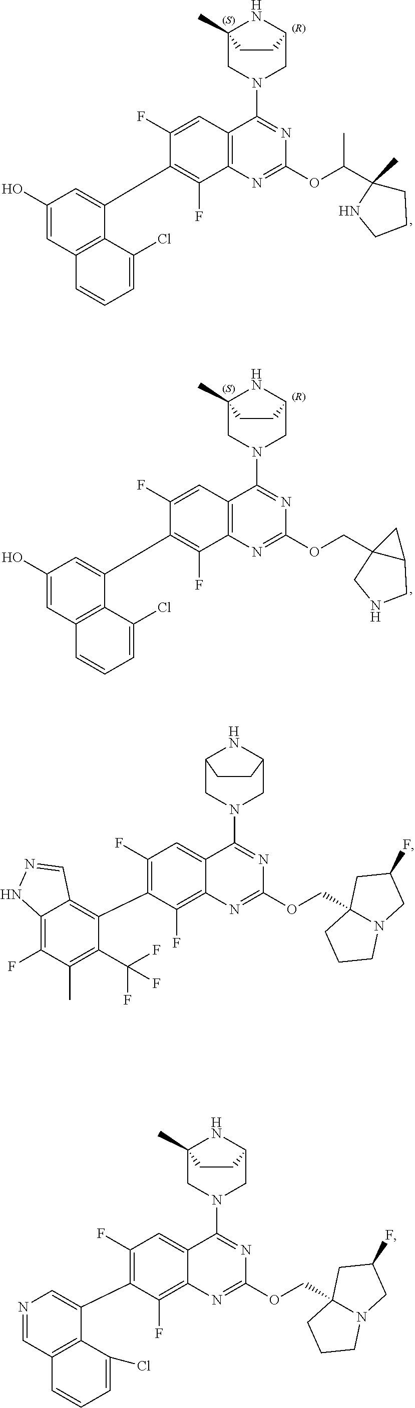 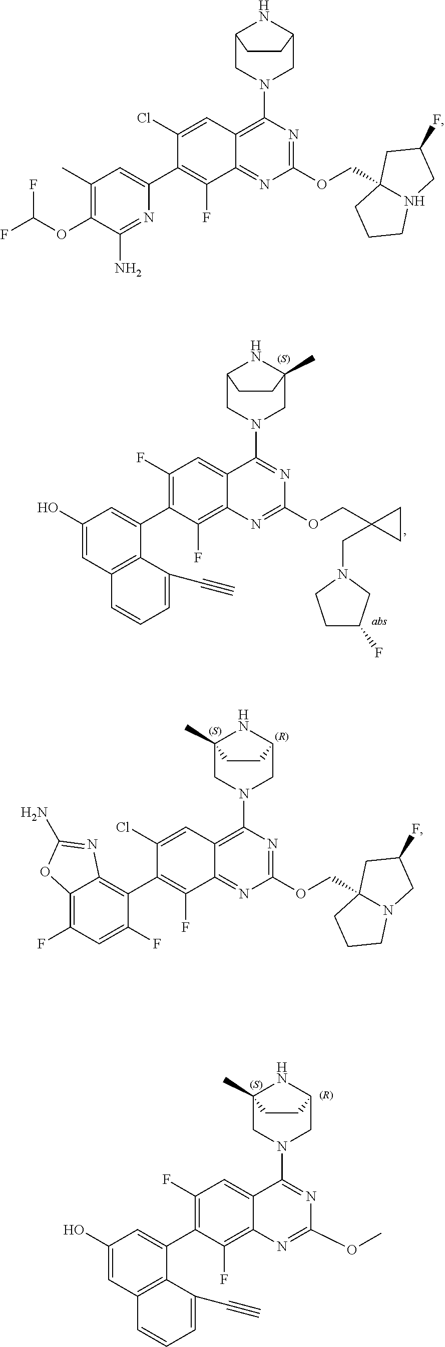 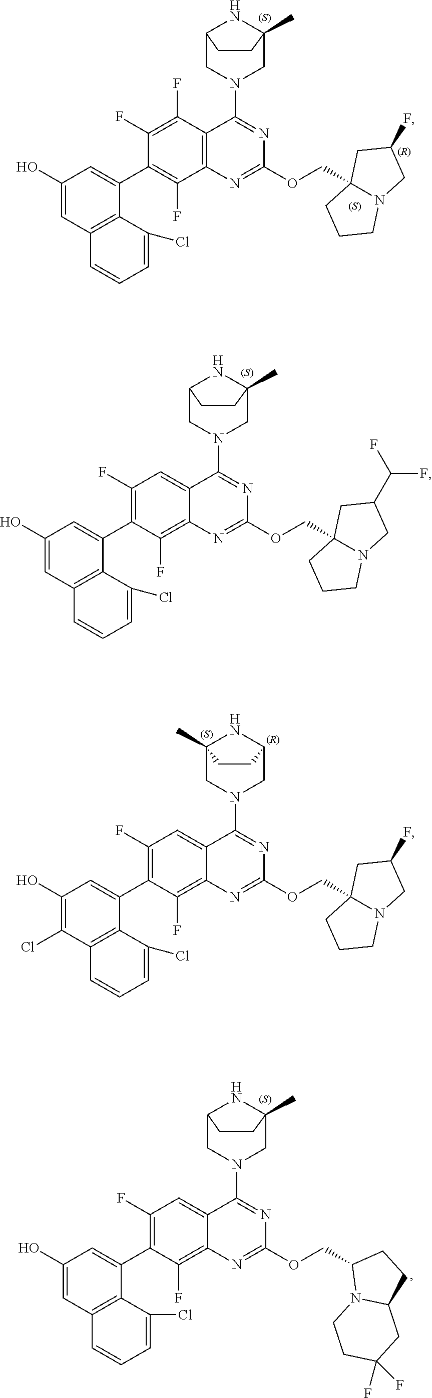 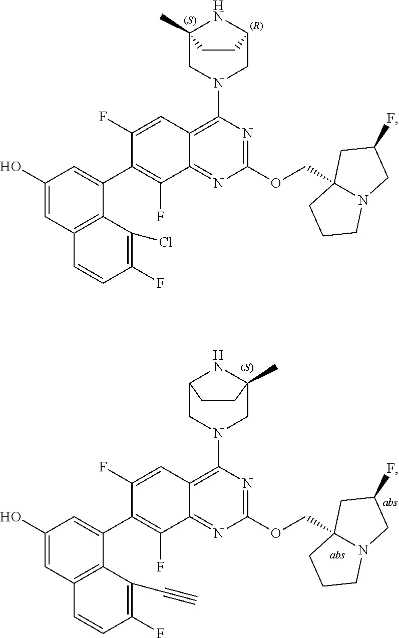 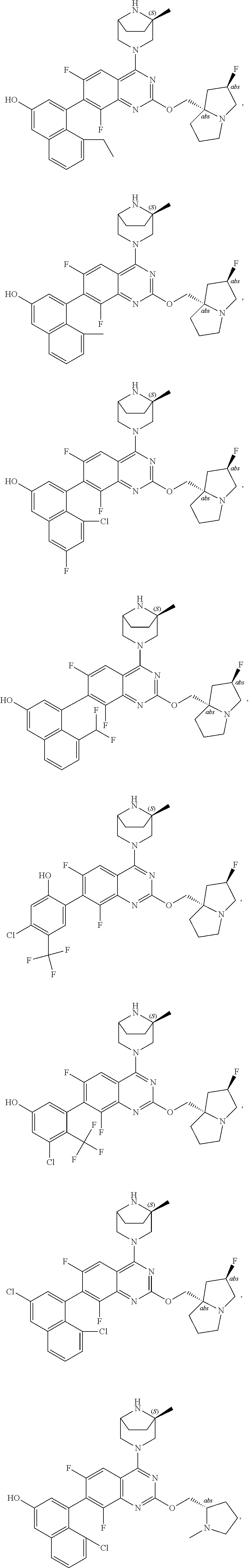 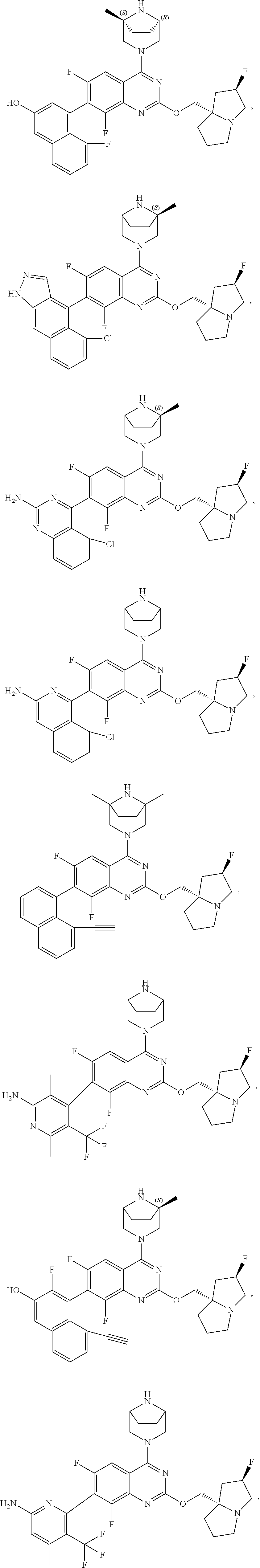 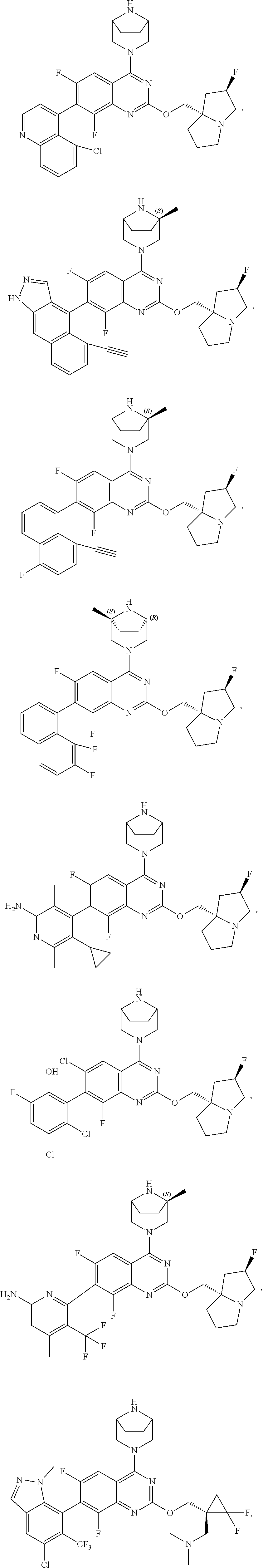 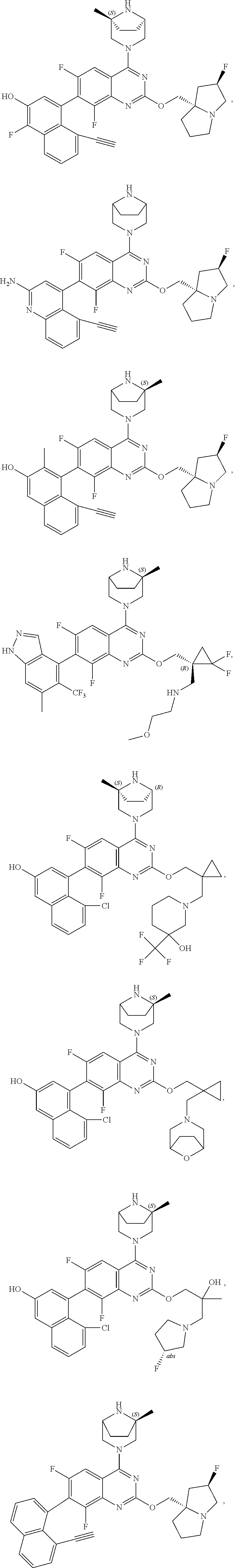 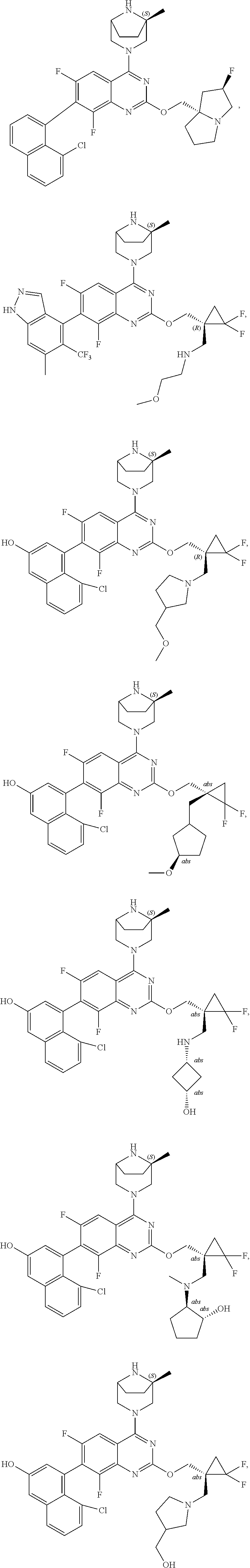 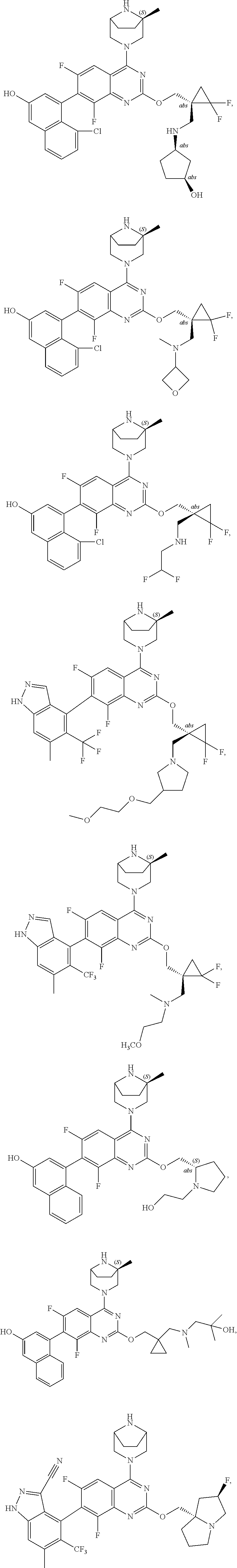 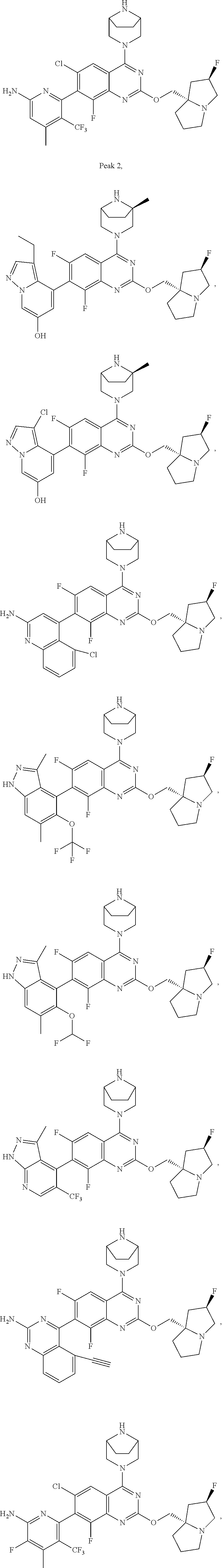 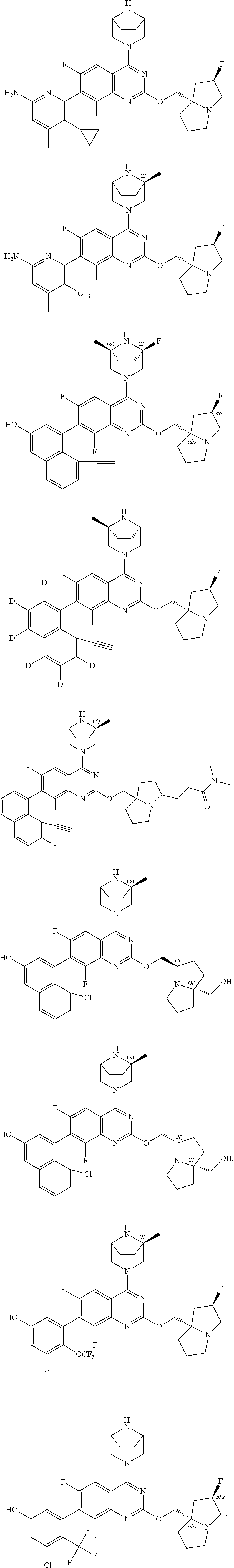 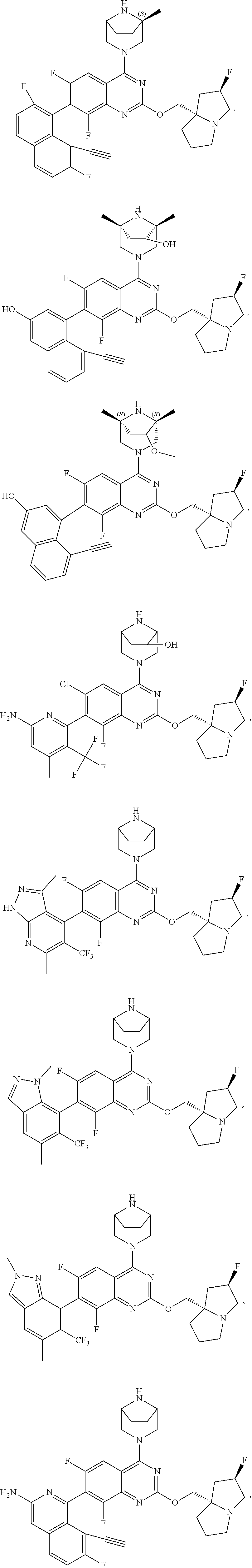 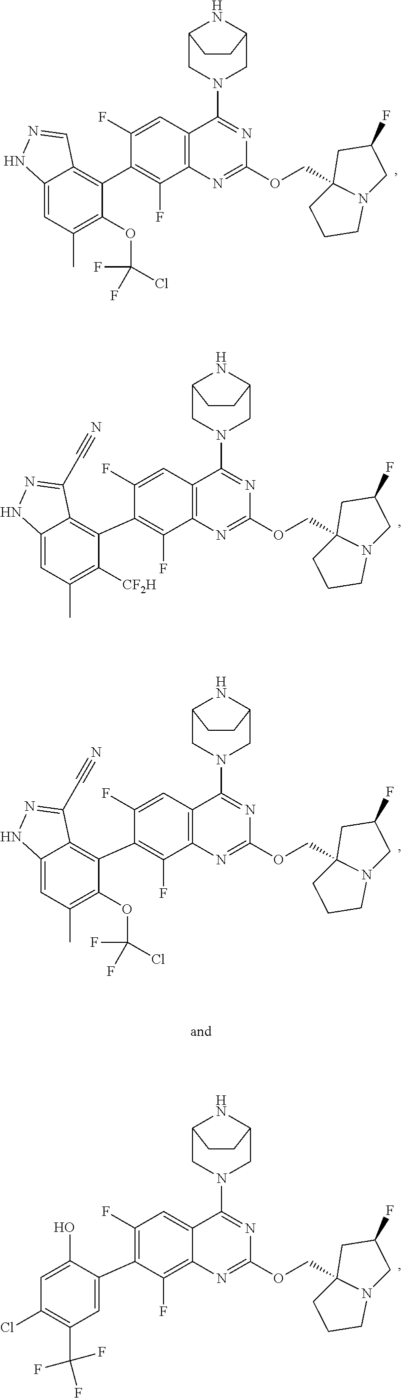 [SMILES: Cc1cc2[nH]ncc2c(-c2c(F)cc3c(N4CC5CCC(C4)N5)nc(OC[C@@]45CCCN4C[C@H](F)C5)nc3c2F)c1OC(F)(F)Cl ; Cc1cc2[nH]nc(C#N)c2c(-c2c(F)cc3c(N4CC5CCC(C4)N5)nc(OC[C@@]45CCCN4C[C@H](F)C5)nc3c2F)c1C(F)F ; Cc1cc2[nH]nc(C#N)c2c(-c2c(F)cc3c(N4CC5CCC(C4)N5)nc(OC[C@@]45CCCN4C[C@H](F)C5)nc3c2F)c1OC(F)(F)Cl ; Oc1cc(Cl)c(C(F)(F)F)cc1-c1c(F)cc2c(N3CC4CCC(C3)N4)nc(OC[C@@]34CCCN3C[C@H](F)C4)nc2c1F]
or a pharmaceutically acceptable salt thereof.

2. A compound of claim 1 or a pharmaceutically acceptable salt thereof, wherein the compound is selected from the group consisting of:

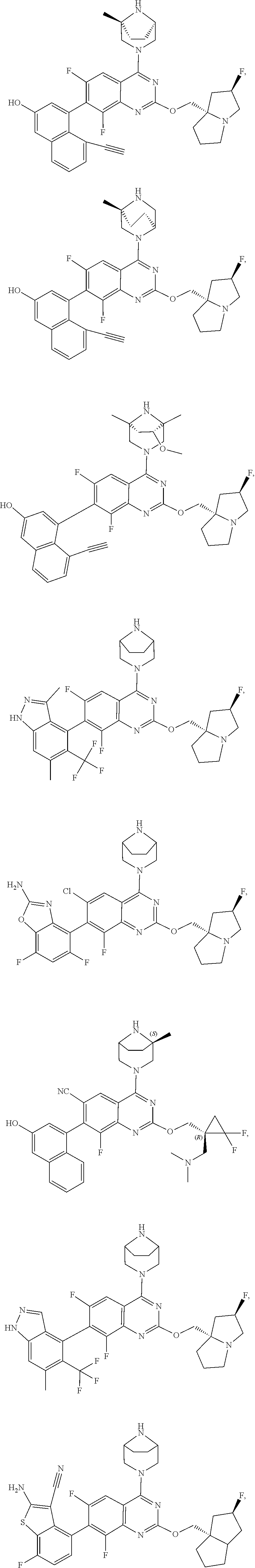

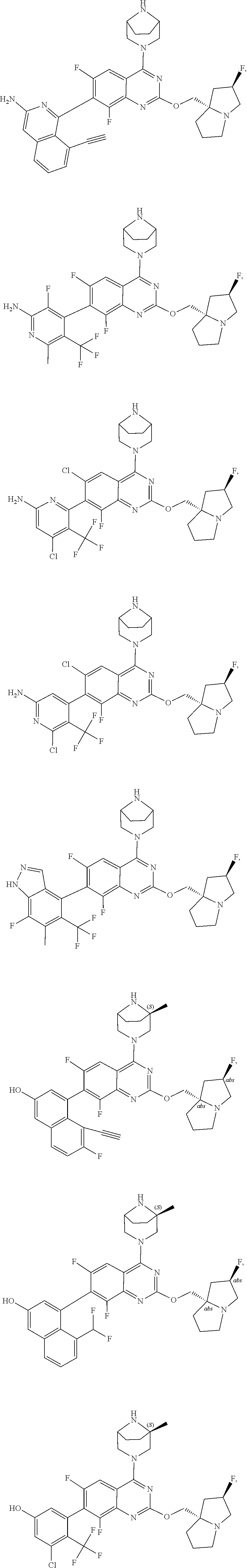

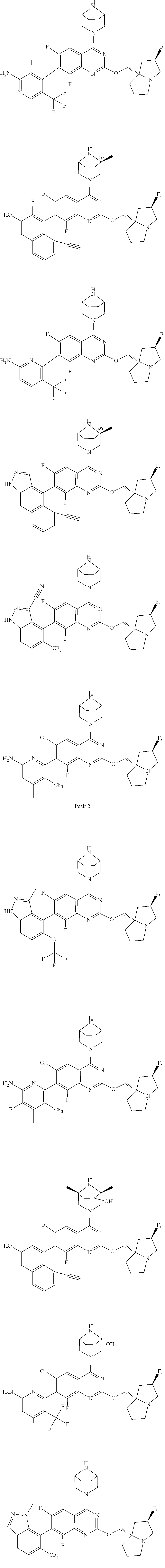

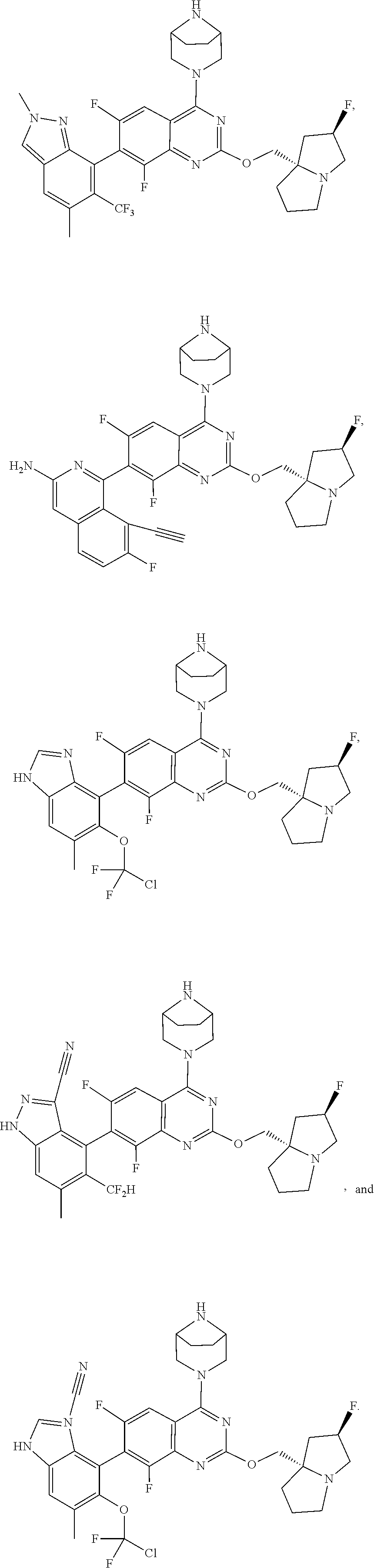

3. A pharmaceutical composition comprising the compound of claim 1 or the pharmaceutically acceptable salt thereof, and a pharmaceutically acceptable carrier.

4. A pharmaceutical composition comprising the compound of claim 1 or the pharmaceutically acceptable salt thereof, an additional anti-cancer agent, and a pharmaceutically acceptable carrier.

5. A method of inhibiting KRAS G12D protein comprising contacting KRAS G12D protein with the compound of claim 1 or the pharmaceutically acceptable salt thereof, to inhibit the activity of the KRAS G12D protein.

6. A method of treating cancer comprising administering a therapeutically effective amount of the compound of claim 1 or the pharmaceutically acceptable salt thereof, to a subject in need of such treatment.

7. The method of claim 6, further comprising administering an additional active agent to the subject.

8.-14. (canceled)


SEQUENCE LISTING (1 sequence)

SEQ ID NO: 1 - protein, 183 aa, Artificial
  Synthetic Protein
GLNDIFEAQKIEWHETEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK